SEQUENCE LISTING (4 sequences)

SEQ ID NO: 1 - protein, 13 aa, Artificial Sequence
  Description of Artificial Sequence Synthetic peptide
ICVVQDWGHHCRT

SEQ ID NO: 2 - protein, 13 aa, Artificial Sequence
  Description of Artificial Sequence Synthetic peptide
  MOD_RES (4)..(4): 1-methyltryptophan
ICVWQDWGAHRCT

SEQ ID NO: 3 - protein, 14 aa, Artificial Sequence
  Description of Artificial Sequence Synthetic peptide
  MOD_RES (1)..(1): D-Tyrosine
  MOD_RES (5)..(5): 1-methyltryptophan
  MOD_RES (9)..(9): Sarcosine
  MOD_RES (14)..(14): N-methylisoleucine
YICVWQDWXAHRCI

SEQ ID NO: 4 - protein, 14 aa, Artificial Sequence
  Description of Artificial Sequence Synthetic peptide
  MOD_RES (1)..(1): PEG-D-Tyrosine
  MOD_RES (5)..(5): 1-methyltryptophan
  MOD_RES (9)..(9): Sarcosine
  MOD_RES (14)..(14): N-methylisoleucine
YICVWQDWXAHRCI

(19) United States
(12) Patent Application Publication
(10) Pub. No.: US 2025/0171423 A1
(43) Pub. Date: May 29, 2025

(54) PHARMACEUTICAL COMPOUNDS FOR THE TREATMENT OF COMPLEMENT MEDIATED DISORDERS

(71) Applicant: Achillion Pharmaceuticals, Inc., Boston, MA (US)

(72) Inventors: Jason Allan Wiles, Madison, CT (US); Venkat Rao Gadhachanda, Hamden, CT (US); Evans O. Onyango, Hamden, CT (US)

(73) Assignee: Achillion Pharmaceuticals, Inc., Boston, MA (US)

(21) Appl. No.: 18/027,506

(22) PCT Filed: Sep. 22, 2021
(86) PCT No.: PCT/US2021/051559

§ 371 Date: Mar. 21, 2023

Related U.S. Application Data
(60) Provisional application No. 63/082,169, filed on Sep. 23, 2020.

(51) Int. Cl. C07D 409/12; A61K 31/4025; A61K 31/407; A61K 31/4178; A61K 31/426; A61K 31/4725; A61K 31/5377; C07D 409/14; C07D 417/12; C07D 417/14; C07D 471/04; C07D 487/08; C07D 491/107; C07D 491/113

(52) U.S. Cl. CPC C07D 409/12; A61K 31/4025; A61K 31/407; A61K 31/4178; A61K 31/426; A61K 31/4725; A61K 31/5377; C07D 409/14; C07D 417/12; C07D 417/14; C07D 471/04; C07D 487/08; C07D 491/107; C07D 491/113

(57) ABSTRACT

This disclosure provides pharmaceutical compounds to treat medical disorders, such as complement-mediated disorders, including complement C1-mediated disorders.

### FIELD OF THE DISCLOSURE

Herein are provided pharmaceutical compounds to treat medical disorders, such as complement-mediated disorders, including complement C1-mediated disorders.

### BACKGROUND OF THE DISCLOSURE

The complement system is a part of the innate immune system which does not adapt to changes over the course of the host's life, but is recruited and used by the adaptive immune system. For example, it assists, or complements, the ability of antibodies and phagocytic cells to clear pathogens. This sophisticated regulatory pathway allows rapid reaction to pathogenic organisms while protecting host cells from destruction. Over thirty proteins and protein fragments make up the complement system. These proteins act through opsonization (enhancing phagocytosis of antigens), chemotaxis (attracting macrophages and neutrophils), cell lysis (rupturing membranes of foreign cells), and agglutination (clustering and binding of pathogens together).

The complement system has three pathways: classical, alternative, and lectin. The classical pathway is triggered by antibody-antigen complexes with the antibody isotypes IgG and IgM. The antibody-antigen complex binds to C1 and this initiates the cleavage of C4 and C2 to generate C3 convertase that then splits C3 into C3a and C3b. C3a interacts with its C3a receptor to recruit leukocytes, while C3b binds to C3 convertase to form C5 convertase. C5 convertase cleaves C5 into C5a and C5b. Similarly to C3a, C5a interacts with its C5a receptor to recruit leukocytes, but C5b interacts with C6, C7, C8, and C8 and together these proteins form the cylindrical membrane attack complex (MAC) that causes the cell to swell and burst. These immune responses can be inhibited by preventing C1 from being able to bind the antibody-antigen complex.

Given the range of serious diseases mediated by a disfunction of the complement system, there is a clear medical need to provide pharmaceutically acceptable compounds, methods, compositions, and methods of manufacture to inhibit the complement system in a patient in need thereof.

Therefore, the present disclosure provides compounds and their uses and compositions to treat disorders arising from or amplified by a disfunction of the complement system. The present disclosure also provides compounds, uses, compositions, combinations, and processes of manufacture that inhibit C1s (complement 1 esterase) and thus can treat disorders mediated by C1s.

### SUMMARY

This disclosure includes a compound of Formula I, II, III, IV, V, VI, VII, VIII, IX, X, XI, XII, XIII, XIV, XV, XVI, XVII, XVIII, XIX, or XX, or a pharmaceutically acceptable salt, prodrug, N-oxide, or isolated isomer thereof, optionally in a pharmaceutically acceptable composition. In one embodiment, the compound or its salt or composition, as described herein is used to treat a medical disorder which is an inflammatory or immune condition, a disorder mediated by the complement cascade (including a dysfunctional cascade), a disorder or abnormality of a cell that adversely affects the ability of the cell to engage in or respond to normal complement activity including for example, the classical complement pathway, or an undesired complement-mediated response to a medical treatment, such as surgery or other medical procedure or a pharmaceutical or biopharmaceutical drug administration, a blood transfusion, or other allogenic tissue or fluid administration.

These compounds can be used to treat medical conditions in a host in need thereof, typically a human. The active compound may act as an inhibitor of the complement classical pathway by inhibiting complement C1s. In one embodiment, a method for the treatment of a disorder mediated by complement activity is provided that includes the administration of an effective amount of a compound of Formula I, II, III, IV, V, VI, VII, VIII, IX, X, XI, XII, XIII, XIV, XV, XVI, XVII, XVIII, XIX, or XX, or a pharmaceutically acceptable salt, prodrug, N-oxide, or isolated isomer thereof, optionally in a pharmaceutically acceptable composition, as described in more detail below.

In one embodiment, the disorder is associated with the complement classical pathway and the compound inhibits the classical pathway. In yet another embodiment, the disorder is associated with the alternative complement cascade pathway. In a further embodiment, the disorder is associated with the complement lectin pathway. Alternatively, the active compound or its salt or prodrug may act through a different mechanism of action than the complement cascade to treat a disorder described herein. In another embodiment, the active compound, and/or its salt or prodrug, inhibits a combination of these pathways.

In another embodiment, a method is provided for treating a host, typically a human, with a disorder mediated by the complement system, that includes administration of a prophylactic antibiotic or vaccine to reduce the possibility of a bacterial infection during the treatment using one of the compounds described herein. In certain embodiments, the host, typically a human, is given a prophylactic vaccine prior to, during or after treatment with one of the compounds described herein. In certain embodiments, the host, typically a human, is given a prophylactic antibiotic prior to, during or after treatment with one of the compounds described herein. In some embodiments, the infection is a meningococcal infection (e.g., septicemia and/or meningitis), an Aspergillus infection, or an infection due to an encapsulated organism, for example, Streptococcus pneumoniae or Haemophilus influenza type b (Hib), especially in children. In other embodiments, the vaccine or antibiotic is administered to the patient after contracting an infection due to, or concomitant with, inhibition of the complement system.

In one aspect of the present disclosure, a compound of Formula I, II, III, IV, V, VI, VII, VIII, or IX is provided:

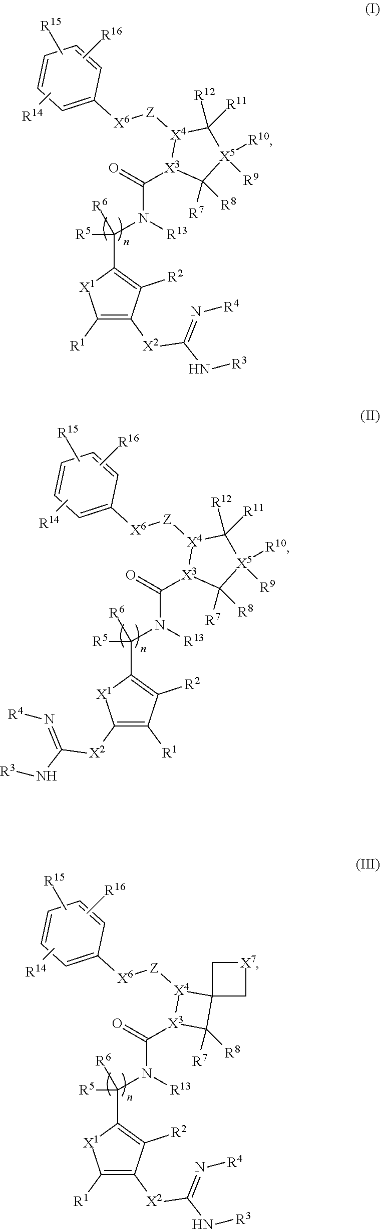

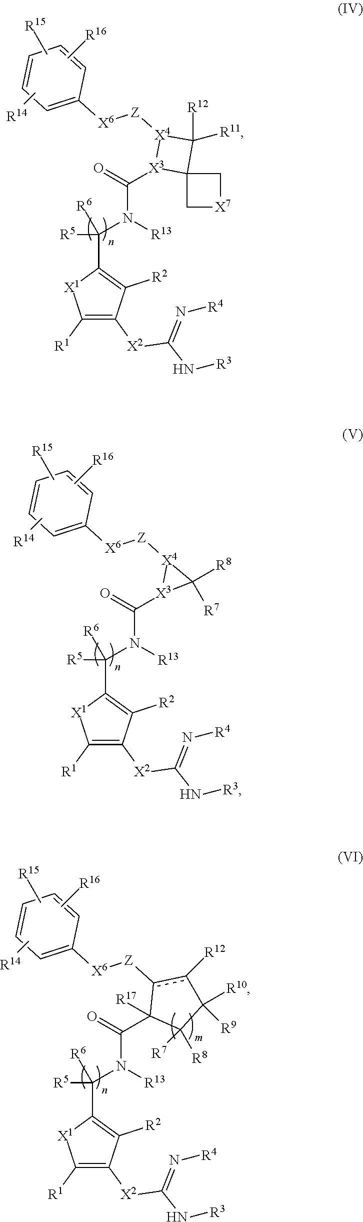

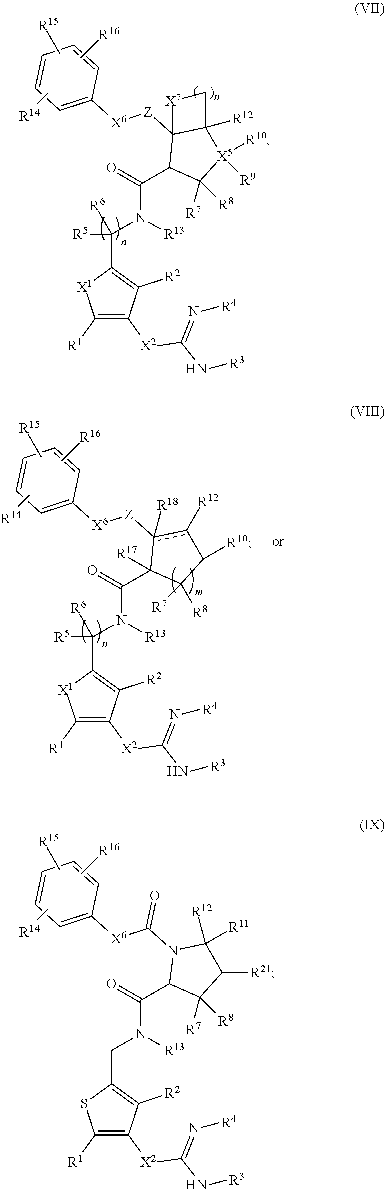

or a pharmaceutically acceptable salt, prodrug, or isolated isomer thereof optionally in pharmaceutically acceptable carrier;
wherein:
each n is independently 0, 1, 2, or 3;
each m is independently 0, 1, 2, or 3;

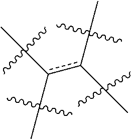

is either a single or a double bond;
Z is CH₂, C(CH₂), or C(O);
X¹ is selected from S, O, and N(R³⁰);
X² is selected from bond, N(R³⁰), and —O—N(R³⁰)—;
X³ is selected from N and C(R¹⁷);
X⁴ is selected from N and C(R¹⁸);
wherein only one of X³ and X⁴ can be N;
X⁵ is C, Si, or S;
X⁶ is selected from

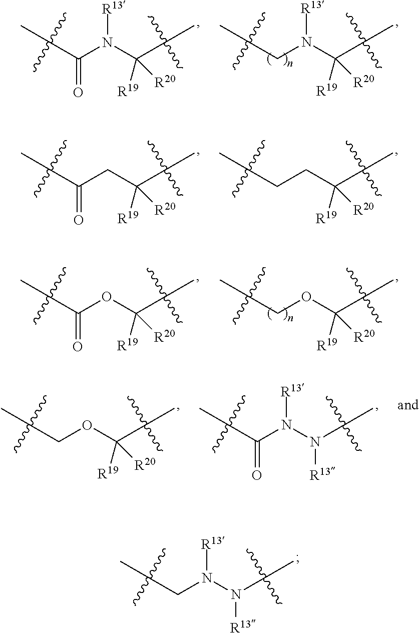

X⁷ is selected from O, S, N(R³⁰), and CR⁵R⁶;
R¹ and R² are independently selected from hydrogen, halogen, C₁-C₆ alkyl, C₂-C₆ alkenyl, C2-C₆ alkynyl, C₁-C₆ haloalkyl, —OR³⁰, —SR³⁰, —N(R³⁰)₂, —C(O)R³¹, —S(O)R³¹, —S(O)₂R³¹, heterocycle, aryl, heteroaryl, cyano, and nitro; each of which R¹ and R² groups other than hydrogen, halogen, cyano, and nitro are optionally substituted with 1, 2, 3, or 4 substituents independently selected from C₁-C₆ alkyl, C₂-C₆ alkenyl, C₂-C₆ alkynyl, halogen, C₁-C₆ haloalkyl, —OR³⁰, —SR³⁰, —N(R³⁰)₂, —C(O)R³¹, —S(O)R³¹, —S(O)₂R³¹, heterocycle, aryl, heteroaryl, cyano, and nitro;
R³ and R⁴ are independently selected from hydrogen, C(O)R³¹, —SR³⁰, and —OR³⁰;
or R³ and R⁴ are independently selected from hydrogen, CN, C(O)R³¹, —SR³⁰, and —OR³⁰;
or R³ and R⁴ are instead combined to form an dihydrooxadiazole optionally substituted with 1, 2, or 3 substituents independently selected from C₁-C₆ alkyl, C1-C₆ haloalkyl, —OR³⁰, and oxo; an oxadiazole optionally substituted with 1, 2, or 3 substituents independently selected from C₁-C₆alkyl, C₁-C₆haloalkyl, and —OR³⁰; an imidazole optionally substituted with 1, 2, or 3 substituents independently selected from C₁-C₆alkyl, C₁-C₆haloalkyl, and —OR³⁰; or a dihydroimidazole optionally substituted 1, 2, or 3 substituents independently selected from C₁-C₆alkyl, C₁-C₆haloalkyl, —OR³⁰, and oxo; for example,

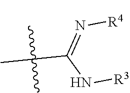

can be

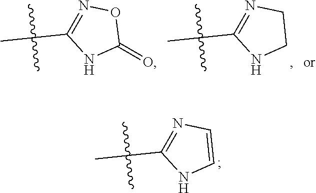 [SMILES: *c1noc(=O)[nH]1 ; *C1=NCCN1 ; *c1ncc[nH]1]

each R⁵ and R⁶ are independently selected from hydrogen, halogen, C₁-C₆ alkyl, C1-C₆ haloalkyl, —OR³⁰, and —N(R³⁰)₂, wherein when on carbons adjacent to each other a R⁵ and a R⁶ group may optionally be replaced by a carbon-carbon double bond, for example,

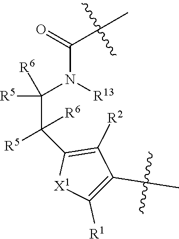

optionally includes

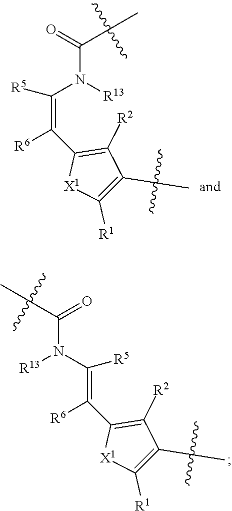

or, when n is 1, R⁵ and R⁶, together with the carbon to which they are attached, are replaced with —SO₂—, for example;
or R⁵ and R⁶, together with the carbon atom to which they are attached, combine to form cyclopropyl;
R⁷, R⁸, R⁹, R¹⁰, R¹¹, and R¹² are independently selected from hydrogen, halogen, C₁-C₆alkyl, C₂-C₆alkenyl, C₂-C₆alkynyl, C₁-C₆haloalkyl, —OR³⁰, —SR³⁰, —N(R³⁰)₂, —C(O)R³¹, —S(O)R³¹, —S(O)₂R³¹, —S(O)(NR³¹)(R³¹), carbocycle, heterocycle, aryl, and heteroaryl, each of which R⁷, R⁸, R⁹, R¹⁰, R¹¹, and R¹² groups other than hydrogen and halogen are optionally substituted with 1, 2, 3, or 4 substituents independently selected from C₁-C₆alkyl, C₂-C₆alkenyl, C₂-C₆alkynyl, halogen, C1-C6haloalkyl, —OR³⁰, —SR³⁰, —N(R³⁰)₂, —C(O)R³¹, —S(O)R³¹, —S(O)₂R³¹, heterocycle, aryl, heteroaryl, cyano, nitro, and azido;
or R⁷ and R⁸ may be taken together with the carbon to which they are attached to form a 3- to 6-membered carbocyclic spiro ring or a 4- to 6-membered heterocyclic spiro ring containing 1 or 2 heteroatoms independently chosen from N, O, and S, are optionally substituted with one or more halogen, C₁-C₆alkyl, C1-C₆ haloalkyl, —OR³⁰, —SR³⁰, or —N(R³⁰)₂;
or R⁷ and R⁸ may be taken together with the carbon to which they are attached to form

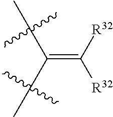

carbonyl;
or R⁹ and R¹⁰ may be taken together with the atom to which they are attached to form a 3- to 6-membered carbocyclic spiro ring or a 4- to 6-membered heterocyclic spiro ring containing 1 or 2 heteroatoms independently chosen from N, O, and S are optionally substituted with one or more halogen, C₁-C₆alkyl, C₁-C₆ haloalkyl, —OR³⁰, —SR³⁰, or —N(R³⁰)₂;
or R⁹ and R¹⁰ may be taken together with the atom to which they are attached to form

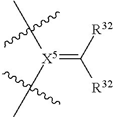

or carbonyl;
or R¹¹ and R¹² may be taken together with the carbon to which they are attached to form a 3- to 6-membered carbocyclic spiro ring or a 4- to 6-membered heterocyclic spiro ring containing 1 or 2 heteroatoms independently chosen from N, O, and S are optionally substituted with one or more halogen, C₁-C₆alkyl, C₁-C₆ haloalkyl, —OR³⁰, —SR³⁰, or —N(R³⁰)₂;
or R¹¹ and R¹² may be taken together with the carbon to which they are attached to form

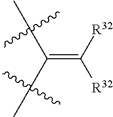

or carbonyl;
or R⁷ and R⁹ are taken together with the atoms to which they are attached to form a 3- to 8-membered carbocycle or a 4- to 8-membered heterocycle containing 1 or 2 heteroatoms independently chosen from N, O, and S;
or R⁹ and R¹¹ are taken together with the atoms to which they are attached to form a 3- to 8-membered carbocycle or a 4- to 8-membered heterocycle containing 1 or 2 heteroatoms independently chosen from N, O, and S;
or R⁷ and R¹¹ are taken together with the atoms to which they are attached to form a 1 or 2 carbon bridge, for example

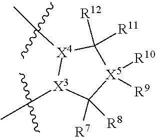

can optionally be

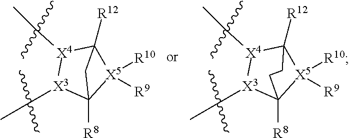

each R¹³ is independently selected from hydrogen, C₁-C₆alkyl, and OH;
or R¹³, together with the nitrogen atom to which it is attached, is replaced with —O—;
each R^{13′} and R^{13″} is independently selected from hydrogen and C₁-C₆alkyl;
or R^{13′} and R¹⁴, together with the atoms to which they are attached, combine to form a 5- or 6-membered heterocycle containing one N;
R¹⁴, R¹⁵, and R¹⁶ are independently selected from hydrogen, halogen, SF₅, C₁-C₆alkyl, C₂-C₆alkenyl, C₂-C₆alkynyl, C₁-C₆haloalkyl, —C₁-C₆alkyl-aryl. —OR³⁰, —SR³⁰, —N(R³⁰)₂, —C(O)R³¹, —S(O)R³¹, —S(O)₂R³¹, carbocycle, heterocycle, aryl, heteroaryl, cyano, and nitro each of which R¹⁴, R¹⁵, and R¹⁶ groups other than hydrogen, halogen, cyano, and nitro are optionally substituted with 1, 2, 3, or 4 substituents independently selected from SF₅, C₁-C₆alkyl, C₂-C₆alkenyl, C₂-C₆alkynyl, halogen, C1-C6haloalkyl, —OR³⁰, —SR³⁰, —N(R³⁰)₂, —C(O)R³¹, —S(O)R³¹, —S(O)₂R³¹, carbocycle, heterocycle, aryl, heteroaryl, cyano, and nitro;
or R¹⁴, R¹⁵, and R¹⁶ are independently selected from hydrogen, halogen, C₁-C₆alkyl, C2-C6alkenyl, C₂-C₆alkynyl, C₁-C₆haloalkyl, —C₁-C₆alkyl-aryl. —OR³⁰, —SR³⁰, —N(R³⁰)₂, —C(O)R³¹, —S(O)R³¹, —S(O)₂R³¹, carbocycle, heterocycle, aryl, heteroaryl, cyano, and nitro each of which R¹⁴, R¹⁵, and R¹⁶ groups other than hydrogen, halogen, cyano, and nitro are optionally substituted with 1, 2, 3, or 4 substituents independently selected from C₁-C₆alkyl, C₂-C₆alkenyl, C₂-C₆alkynyl, halogen, C₁-C₆haloalkyl, —OR³⁰, —SR³⁰, —N(R³⁰)₂, —C(O)R³¹, —S(O)R³¹, —S(O)₂R³¹, carbocycle, heterocycle, aryl, heteroaryl, cyano, and nitro; R¹⁷ and R¹⁸ are independently selected from hydrogen, halogen, C₁-C₆ alkyl,
   C₁-C₆ haloalkyl, —OR³⁰, and —N(R³⁰)₂;
or R¹⁷ and R¹⁸ are taken together with the carbons to which they are attached to form a double bond;
R¹⁹ and R²⁰ are independently selected from hydrogen, C₁-C₆alkyl, C₅-C₁₀ bicyclic carbocycle, C₄-C₆ heterocycle, halogen, C₁-C₆ haloalkyl, —OR³⁰, —N(R³⁰)₂, —(CH₂)ₙ—R³³, and

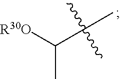

R²¹ is selected from C₁-C₆ alkyl and —O—C₁-C₆ alkyl;
R²¹ is selected from C₁-C₆haloalkyl, —O—C₁-C₆haloalkyl, C₁-C₆alkyl, —O—C₁-C₆alkyl, —S(O)(NR³¹)R³¹, carbocycle, aryl, —O-aryl, heteroaryl, —O-carbocycle, or —O-heteroaryl, each of which R²¹ group is optionally substituted with 1, 2, 3, or 4 substituents independently selected from SF₅, C₁-C₆alkyl, C₂-C₆alkenyl, C₂-C₆alkynyl, halogen, C₁-C₆haloalkyl, —OR³⁰, —SR³⁰, —N(R³⁰)₂, —C(O)R³¹, —S(O)R³¹, —S(O)₂R³¹, heterocycle, aryl, heteroaryl, cyano, nitro, and azido;
each R³⁰ is independently selected from hydrogen, C₁-C₆alkyl, C₁-C₆haloalkyl, carbocycle, aryl, heteroaryl, heterocycle, and C(O)R³¹, each R³⁰ other than C(O)R³¹ is optionally substituted with 1, 2, 3, or 4 substituents selected from C₁-C₆alkyl, halogen, SF₅, —C(O)R³¹, —N(R³⁰)₂, aryl, —OR³², and —S(O)(NR³¹)R³¹;
each R³¹ is independently selected from hydrogen, C₁-C₆ alkyl, C₁-C₆ haloalkyl, —OR³², —SR³², —N(R³²)₂, heterocycle, aryl, and heteroaryl;
each R³² is independently selected from hydrogen, C₁-C₆ alkyl, and C₁-C₆ haloalkyl;
each R³³ is independently selected from hydrogen, guanidine, heteroaryl, aryl, —C₆HS—OR³⁰; —OR³⁰, —SR³⁰, —SeR³⁰, —N(R³⁰)₂, and —C(O)R³¹.

In some embodiments, R⁵ and/or R⁶ may also be C₁-C₆hydroxyalkyl or C(O)R³¹.

In some embodiments, R³⁰ may also be optionally substituted with carbocycle (e.g., cycloalkyl).

In some embodiments, for compounds of Formula I and Formula II at least one of the following is satisfied:
a. X³ is C(R¹⁷) and X⁴ is C(R¹⁸);
b. R¹⁷ is selected from halogen, C₁-C₆ alkyl, C₁-C₆ haloalkyl, —OR³⁰, and —N(R³⁰)₂;
c. X⁵ is Si;
d. X⁵ is S, at least two of R⁷, R⁸, R¹¹, and R¹² are not hydrogen, no more than one of R⁷ and R⁸ is halogen, and no more than one of R¹¹ and R¹² is halogen;
e. Z is C(CH₂);
f. Z is CH₂;
g. R⁷ and R⁸ are taken together with the carbon to which they are attached to form a 3- to 6-membered carbocyclic spiro ring; a 4- to 6-membered heterocyclic spiro ring containing 1 or 2 heteroatoms independently chosen from N, O, and S;

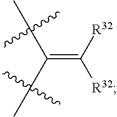

or a carbonyl;
h. R⁹ and R¹⁰ are taken together with the carbon to which they are attached to form a 3- to 6-membered carbocyclic spiro ring; a 4- to 6-membered heterocyclic spiro ring containing 1 or 2 heteroatoms independently chosen from N, O, and S;

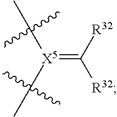

or a carbonyl;
i. R¹¹ and R¹² are taken together with the carbon to which they are attached to form a 3- to 6-membered carbocyclic spiro ring; a 4- to 6-membered heterocyclic spiro ring containing 1 or 2 heteroatoms independently chosen from N, O, and S;

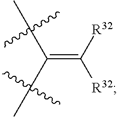

or a carbonyl;
j. R⁷ and R⁹ are taken together with the atoms to which they are attached to form a 3- to 8-membered carbocycle or a 4- to 8-membered heterocycle containing 1 or 2 heteroatoms independently chosen from N, O, and S; and R¹⁰ or R¹² is not hydrogen;
k. R⁹ and R¹¹ are taken together with the atoms to which they are attached to form a 3- to 8-membered carbocycle or a 4- to 8-membered heterocycle containing 1 or 2 heteroatoms independently chosen from N, O, and S; and R⁸ or R¹⁰ is not hydrogen;
l. R⁷ and R¹¹ are taken together with the atoms to which they are attached to form a 1 or 2 carbon bridge;
m. X⁶ is selected from
n. at least one of R³ and R⁴ is CN, —SR³⁰ or C(O)R³¹; or

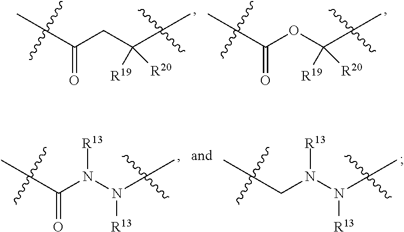

o. R³ and R⁴ are combined to form a dihydroxadiazole optionally substituted with 1, 2, or 3 substituents independently selected from C₁-C₆alkyl, C₁-C₆haloalkyl, —OR³⁰, and oxo; an oxadiazole optionally substituted with 1, 2, or 3 substituents independently selected from C₁-C₆alkyl, C₁-C₆haloalkyl, and —OR³⁰; an imidazole optionally substituted with 1, 2, or 3 substituents independently selected from C₁-C₆alkyl, C₁-C₆haloalkyl, and —OR³⁰; or a dihydroimidazole optionally substituted 1, 2, or 3 substituents independently selected from C₁-C₆alkyl, C₁-C₆haloalkyl, —OR³⁰, and oxo.

In one embodiment, the compound of Formula IX is:

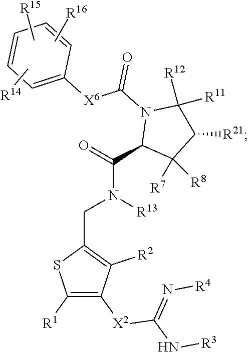

or a pharmaceutically acceptable salt thereof.

In another embodiment, the compound of Formula IX is:

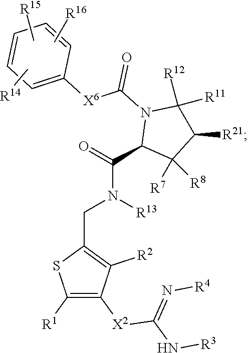

or a pharmaceutically acceptable salt thereof.

In an alternative embodiment, the compound of Formula IX is

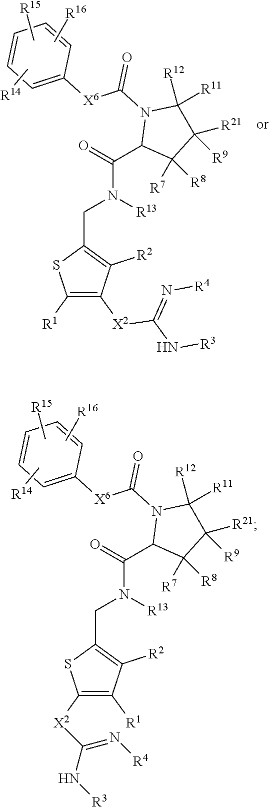

or a pharmaceutically acceptable salt thereof.

In another aspect, the compound of the present disclosure is of Formula X, XI, or XII:

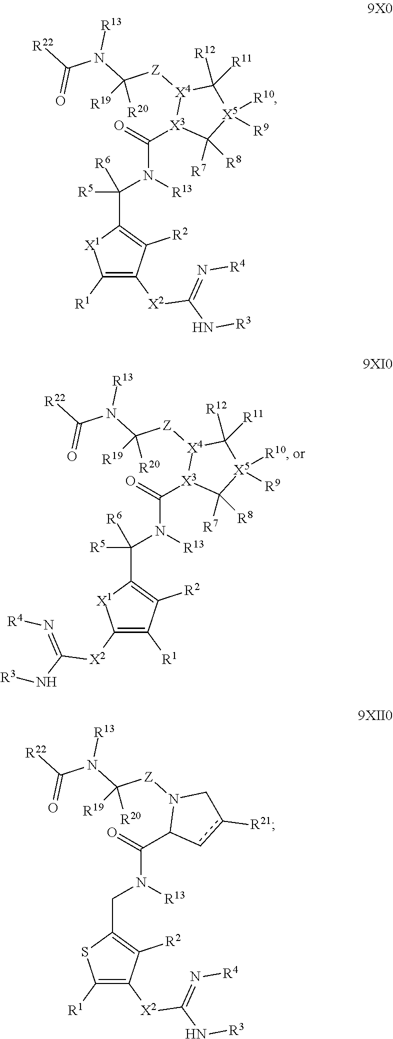

or a pharmaceutically acceptable salt, prodrug, or isolated isomer thereof optionally in pharmaceutically acceptable carrier;
wherein:
R²² is selected from —C₁-C₆alkyl-R²³, —C₂-C₆alkenyl-R²³, —C₂-C₆alkynyl-R²³, -heteroaryl-R²³, -carbocycle-R²³, and bicyclic cycloalkyl-R²³, each of which R²² is optionally substituted with 1, 2, 3, or 4 substituents independently selected from C₁-C₆alkyl, C₂-C₆alkenyl, C₂-C₆alkynyl, halogen, C₁-C₆haloalkyl,
   —OR³⁰, —SR³⁰, —N(R³⁰)₂, —C(O)R³¹, —S(O)R³¹, —S(O)₂R³¹, heterocycle, aryl, heteroaryl, cyano, and nitro;
R²³ is selected from hydrogen, sugar, —OR³⁰, —SR³⁰, —N(R³⁰)₂, —C(O)R³¹, —S(O)R³¹, and —S(O)₂R³¹; and
all other variables are as defined herein;

In some embodiments, for compounds of Formula X and Formula XI, at least one of the following is satisfied:
a. X³ is C(R¹⁷) and X⁴ is C(R¹⁸);
b. R¹⁷ is selected from halogen, C₁-C₆ alkyl, C₁-C₆ haloalkyl, —OR³⁰, and —N(R³⁰)₂;
c. X⁵ is Si;
d. X⁵ is S, at least two of R⁷, R⁸, R¹¹, and R¹² are not hydrogen, no more than one of R⁷ and R⁸ is halogen, and no more than one of R¹¹ and R¹² is halogen;
e. Z is C(CH₂);
f. Z is CH₂;
g. R⁷ and R⁸ are taken together with the carbon to which they are attached to form a 3- to 6-membered carbocyclic spiro ring; a 4- to 6-membered heterocyclic spiro ring containing 1 or 2 heteroatoms independently chosen from N, O, and S;

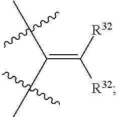

or a carbonyl;
h. R⁹ and R¹⁰ are taken together with the carbon to which they are attached to form a 3- to 6-membered carbocyclic spiro ring; a 4- to 6-membered heterocyclic spiro ring containing 1 or 2 heteroatoms independently chosen from N, O, and S;

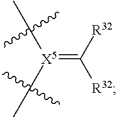

or a carbonyl;
i. R⁹ and R¹¹ are taken together with the atoms to which they are attached to form a 3- to 8-membered carbocycle or a 4- to 8-membered heterocycle containing 1 or 2 heteroatoms independently chosen from N, O, and S; and R¹⁰ is not hydrogen;
j. R¹¹ and R¹² are taken together with the carbon to which they are attached to form a 3- to 6-membered carbocyclic spiro ring; a 4- to 6-membered heterocyclic spiro ring containing 1 or 2 heteroatoms independently chosen from N, O, and S;

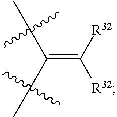

or a carbonyl;
k. R⁷ and R⁹ are taken together with the atoms to which they are attached to form a 3- to 8-membered carbocycle or a 4- to 8-membered heterocycle containing 1 or 2 heteroatoms independently chosen from N, O, and S; and R¹⁰ is not hydrogen;
l. R⁷ and R¹¹ are taken together with the atoms to which they are attached to form a 1 or 2 carbon bridge;
m. R²² is substituted with at least three OR³⁰ groups;
n. R²³ is a sugar;
o. at least one of R³ and R⁴ is CN, —SR³⁰, or C(O)R³¹; or
p. R³ and R⁴ are combined to form an oxadiazole optionally substituted with 1, 2, or 3 substituents independently selected from C₁-C₆ alkyl, C₁-C₆ haloalkyl, —OR³⁰, and oxo.

In one embodiment, R²³ is selected from hydrogen, —OR³⁰, —SR³⁰, —N(R³⁰)₂, —C(O)R³¹, —S(O)R³¹, and —S(O)₂R³¹.

In one embodiment, the compound of Formula X is selected from:

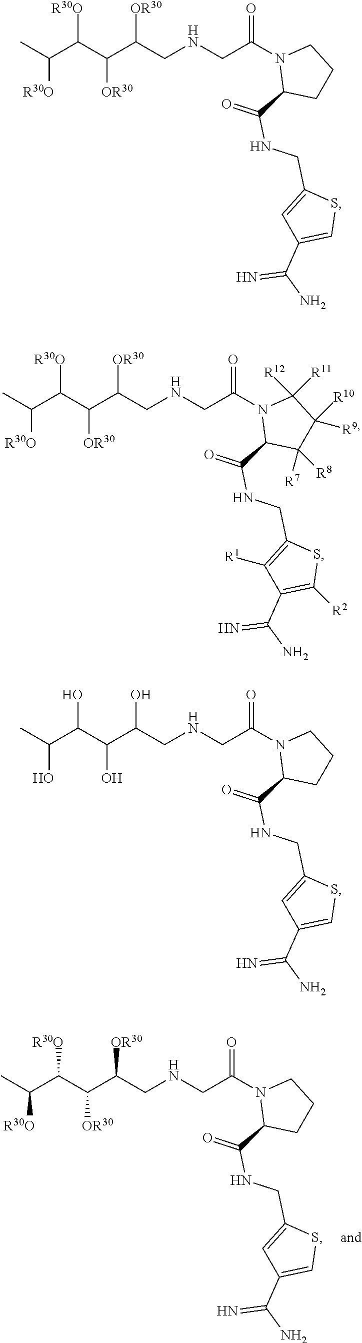

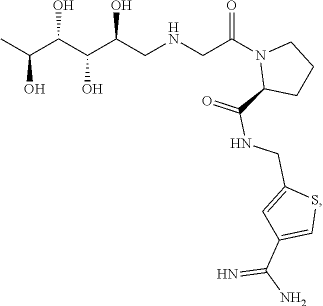 [SMILES: C[C@H](O)[C@H](O)[C@@H](O)[C@@H](O)CNCC(=O)N1CCC[C@H]1C(=O)NCc1cc(C(=N)N)cs1]

In one embodiment, R²³ is selected from —OR³⁰, —SR³⁰, —N(R³⁰)₂, —C(O)R³¹, —S(O)R³¹, and —S(O)₂R³¹.

In another aspect, the compound of Formula X, XI, or XII is selected from

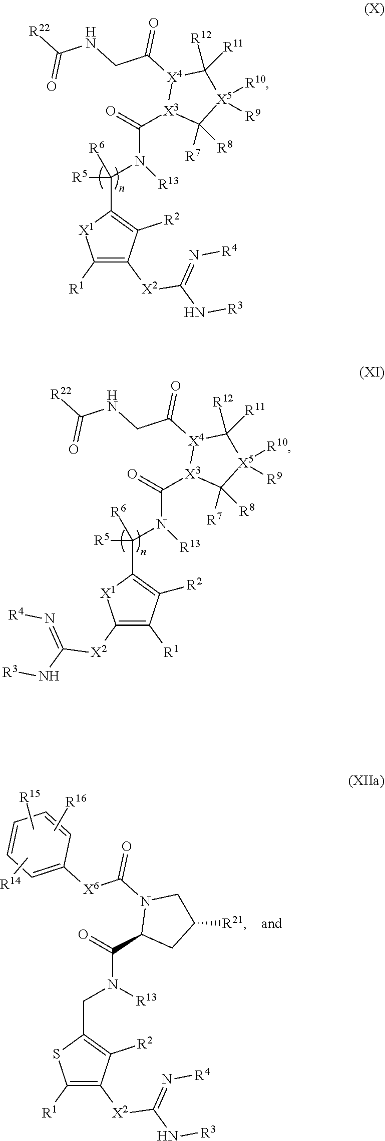

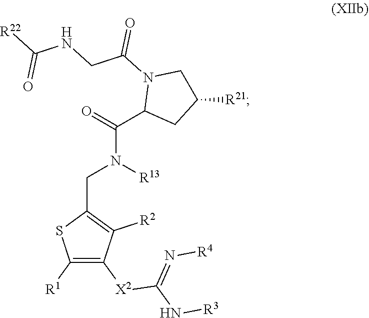

or a pharmaceutically acceptable salt, prodrug, or isolated isomer thereof optionally in pharmaceutically acceptable carrier.

In an alternative embodiment, the compound of Formula XII is

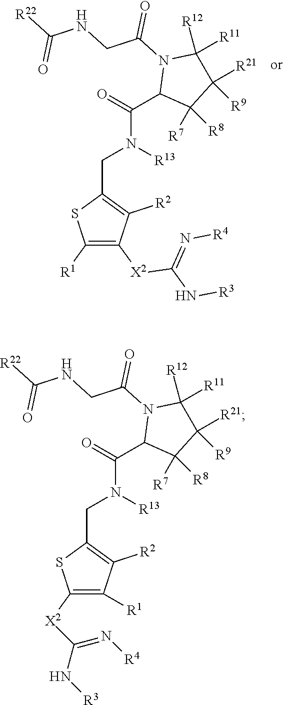

or a pharmaceutically acceptable salt thereof.

In another aspect, the compound of the present disclosure is of Formula XIII:

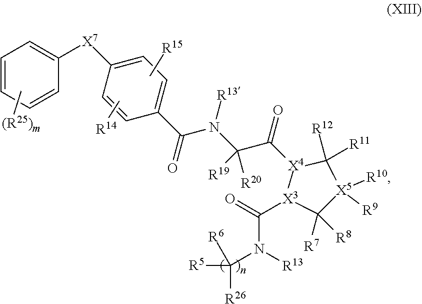

or a pharmaceutically acceptable salt prodrug, or isolated isomer thereof optionally in pharmaceutically acceptable carrier;
wherein:
X⁷ is selected from O, S, N(R³⁰), and CR⁵R⁶;
o is 0, 1, or 2;
each R²⁵ is independently selected from hydrogen, halogen, C₁-C₆ alkyl, C₂-C₆ alkenyl, C₂-C₆ alkynyl, C₁-C₆ haloalkyl, —OR³⁰, —SR³⁰, —N(R³⁰)₂, —C(O)R³¹, —S(O)R³¹, —S(O)₂R³¹, heterocycle, aryl, heteroaryl, cyano, and nitro; each of which R²⁵ groups other than hydrogen, halogen, cyano, and nitro are optionally substituted with 1, 2, 3, or 4 substituents independently selected from C₁-C₆ alkyl, C₂-C₆ alkenyl, C₂-C₆alkynyl, halogen, C₁-C₆haloalkyl, —OR³⁰, —SR³⁰, —N(R³⁰)₂, —C(O)R³¹, —S(O)R³¹, —S(O)₂R³¹, heterocycle, aryl, heteroaryl, cyano, and nitro; or
each R²⁵ is independently selected from hydrogen, SF₅, halogen, C₁-C₆ alkyl, C₂-C₆ alkenyl, C₂-C₆ alkynyl, C₁-C₆ haloalkyl, —OR³⁰, —SR³⁰, —N(R³⁰)₂, —C(O)R³¹, —S(O)R³¹, —S(O)₂R³¹, heterocycle, aryl, heteroaryl, cyano, and nitro; each of which R²⁵ groups other than hydrogen, halogen, cyano, and nitro are optionally substituted with 1, 2, 3, or 4 substituents independently selected from C₁-C₆alkyl, C₂-C₆alkenyl, C₂-C₆alkynyl, halogen, C₁-C₆haloalkyl, —OR³⁰, —SR³⁰, —N(R³⁰)₂, —C(O)R³¹, —S(O)R³¹, —S(O)₂R³¹, heterocycle, aryl, heteroaryl, cyano, and nitro;
R²⁶ is selected from

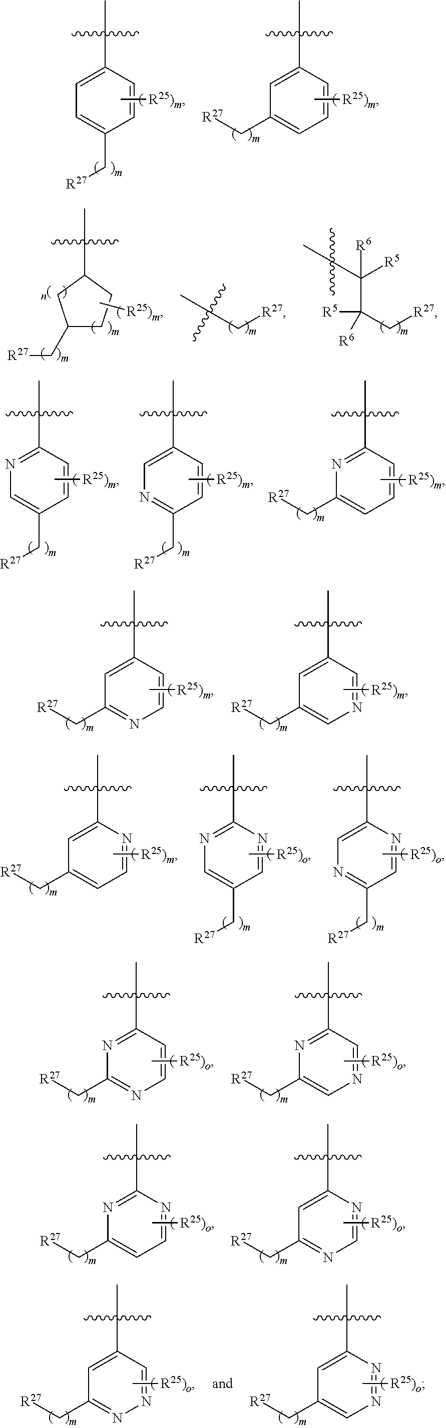

or R²⁶ is selected from m

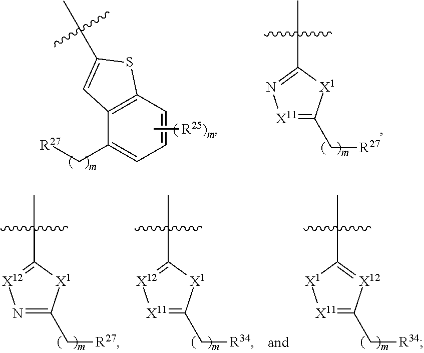

### or R²⁶ is, and

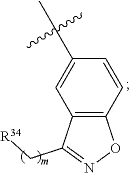

R²⁷ is selected from

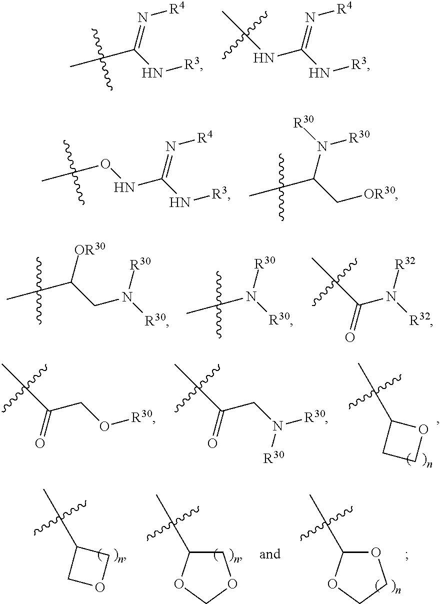

or R²⁷ is

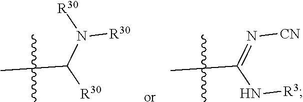

or R²⁷ is

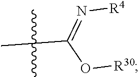

R³⁴ is selected from

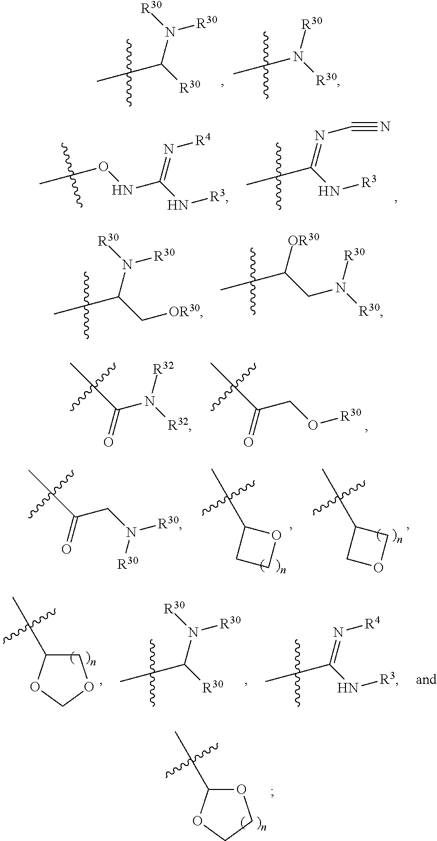

X¹¹ is selected from N and CR¹;
X¹² is selected from N and CR²
wherein each other variable is as defined herein;
or R³⁰ and R₄ in

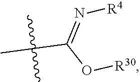

together with the N and O atoms to which each is attached and the carbon atom to which the N and O atoms are attached, combine to form oxadiazole optionally substituted with 1, 2, or 3 substituents independently selected from C₁-C₆alkyl, C₁-C₆haloalkyl, and —OR³⁰;
or R¹³ and R²⁶, together with the atoms to which they are attached, form a heterocycle optionally substituted with R²⁷.

In another aspect, the compound of the present disclosure is of Formula XIV:

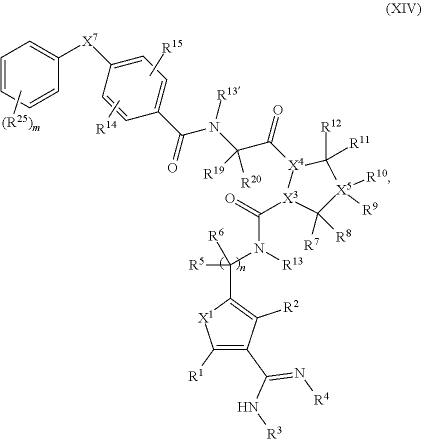

or a pharmaceutically acceptable salt, prodrug, or isolated isomer thereof optionally in pharmaceutically acceptable carrier;
wherein the variables are as defined herein and for compounds of Formula XIV at least one of the following is satisfied:
a. X¹ is O or N(R³⁰);
b. R¹⁴ is not hydrogen;
c. R¹ is not hydrogen;
d. R² is not hydrogen;
e. R³ is not hydrogen; or
f. R⁴ is not hydrogen.

In another aspect the compound of Formula XIII or XIV is selected from

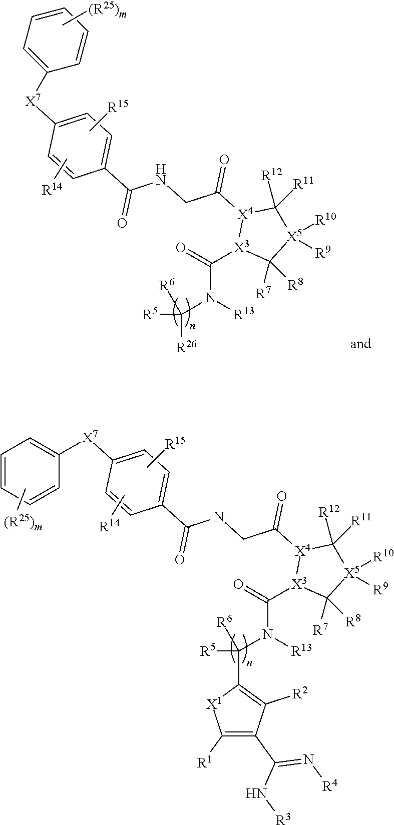

or a pharmaceutically acceptable salt, prodrug, or isolated isomer thereof optionally in pharmaceutically acceptable carrier.

In another aspect, the compound of the present disclosure is of Formula XV:

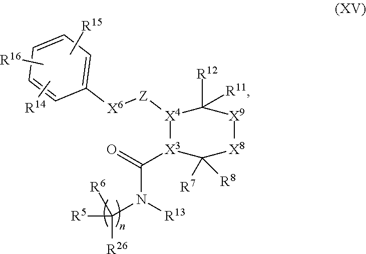

or a pharmaceutically acceptable salt, prodrug, or isolated isomer thereof optionally in pharmaceutically acceptable carrier;
wherein:
each X⁸ and X⁹ is independently selected from O, S, NR³⁰, CR⁹R¹⁰, CR⁵R⁶. and CH₂; wherein X⁸ and X⁹ cannot both be the same group; and all other variables are as defined herein.

In an alternative embodiment

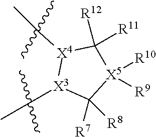

is replaced with

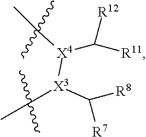

for example in this embodiment the compound of formula

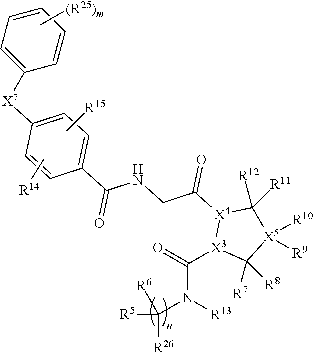

can be replaced with

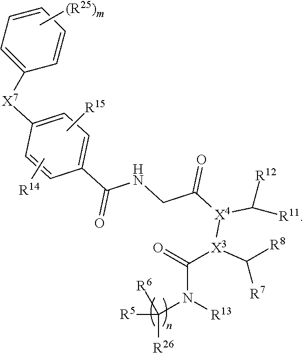

In another aspect, the compound of the present disclosure is of Formula XVI, XVII, or XVIII:

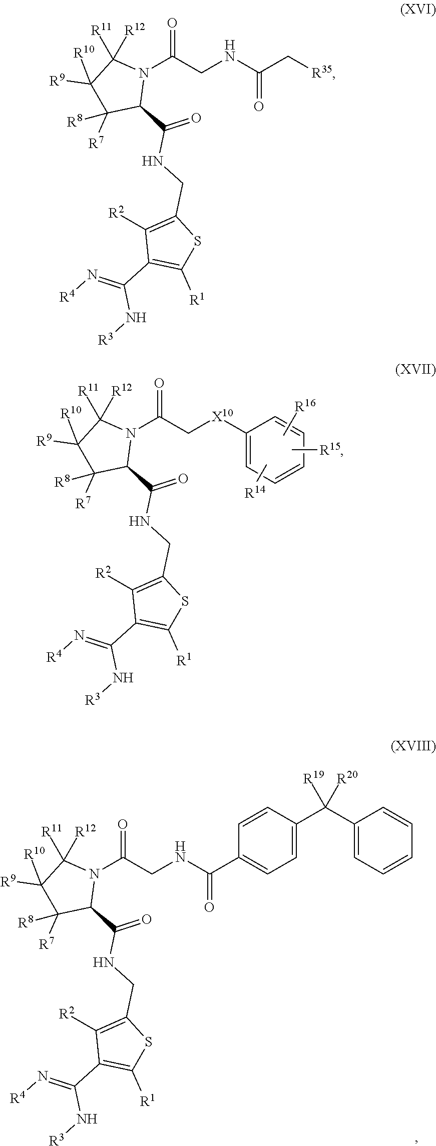

or a pharmaceutically acceptable salt, prodrug, or isolated isomer thereof optionally in pharmaceutically acceptable carrier;
wherein:
X¹⁰ is selected from

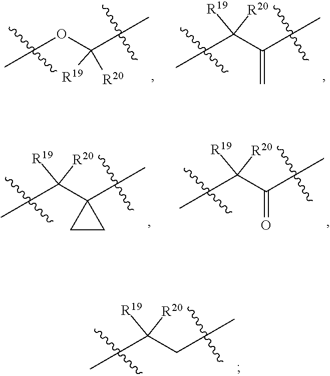

R³⁵ is selected from C₃-C₁₀alkyl or C₃-C₁₀haloalkyl; and
all other variables are as defined herein.

In another aspect, the compound of the present disclosure is of Formula XIX or Formula XX:

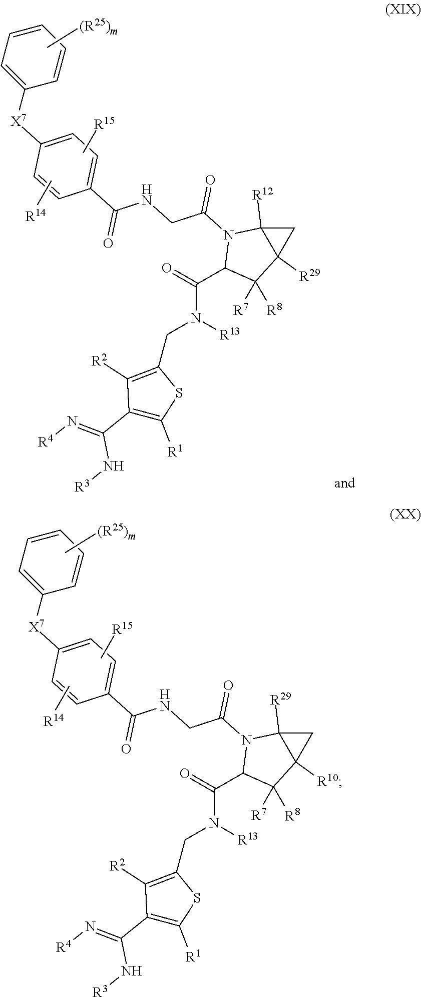

or a pharmaceutically acceptable salt, prodrug, or isolated isomer thereof optionally in pharmaceutically acceptable carrier;
wherein:
R²⁹ is selected from halogen, C₁-C₆alkyl, C₂-C₆alkenyl, C₂-C₆alkynyl, C₁-C₆haloalkyl, —OR³⁰, —SR³⁰, —N(R³⁰)₂, —C(O)R³¹, —S(O)R³¹, —S(O)₂R³¹, heterocycle, aryl, and heteroaryl, each of which R²⁹ groups other than hydrogen and halogen are optionally substituted with 1, 2, 3, or 4 substituents independently selected from C₁-C₆alkyl, C₂-C₆alkenyl, C₂-C₆alkynyl, halogen, C₁-C₆haloalkyl, —OR³⁰, —SR³⁰, —N(R³⁰)₂, —C(O)R³¹, —S(O)R³¹, —S(O)₂R³¹, heterocycle, aryl, heteroaryl, cyano, and nitro; and
all other variables are as defined herein.

In an alternative embodiment R²⁹ is hydrogen.

Pharmaceutical compositions comprising a compound or salt of Formula I, II, III, IV, V, VI, VII, VIII, IX, X, XI, XII, XIII, XIV, XV, XVI, XVII, XVIII, XIX, or XX, together with a pharmaceutically acceptable carrier are also disclosed.

The present disclosure thus includes at least the following features:
a. a compound of the present disclosure or a pharmaceutically acceptable salt, prodrug, N-oxide, or isolated isomer thereof, optionally in a pharmaceutically acceptable composition;
b. a compound of the present disclosure or a pharmaceutically acceptable salt, prodrug, N-oxide, or isolated isomer thereof, optionally in a pharmaceutically acceptable composition, for use in treating or preventing a disorder including but not limited to the development of fatty liver and conditions stemming from fatty liver, such as nonalcoholic steatohepatitis (NASH), liver inflammation, cirrhosis, liver failure; dermatomyositis; amyotrophic lateral sclerosis; cytokine or inflammatory reactions in response to biotherapeutics (e.g., CAR T-cell therapy); hereditary angioedema (HAE), chronic immune thrombocytopenia (ITP), cold agglutinin disease, cold agglutinin syndrome, warm autoimmune hemolytic anemia, cryoglobulinemia, bullous pemphigoid, common variable immunodeficiency, endotoxemia, sepsis, multiple organ dysfunction syndrome, hemolytic uremic syndrome (HUS), atypical hemolytic uremic syndrome (aHUS), acute kidney injury, kidney transplantation, graft rejection, antibody-mediated rejection, delayed graft function, end-stage renal disease, myasthenia gravis, systemic lupus erythema (SLE), paroxysmal nocturnal hemoglobinuria (PNH), rheumatoid arthritis, multiple sclerosis, age-related macular degeneration (AMD), retinal degeneration, other ophthalmic diseases (e.g., geographic atrophy), a respiratory disease or a cardiovascular disease; a disorder of the central nervous system or peripheral nervous system, ischaemic-reperfusion injury or stroke, traumatic brain injury (TBI) and spinal cord injury (SCI), Alzheimer's diseases (AD), multiple sclerosis, neuromyelitis optica (NMO), amyotrophic lateral sclerosis (ALS), Parkinson's disease (PD), Huntington's disease (HD), demyelinating myelinoclastic diseases, demyelinating leukostrophic diseases, and neurological inflammatory disorders;
c. a pharmaceutically acceptable composition of a compound of the present disclosure or its pharmaceutically acceptable salt, prodrug, N-oxide, or isolated isomer thereof in a pharmaceutically acceptable carrier;
d. a compound of the present disclosure or a pharmaceutically acceptable salt, prodrug, N-oxide, or isolated isomer thereof, optionally in a pharmaceutically acceptable composition, for use in treating or preventing a disorder mediated by the complement pathway, and for example, the classical complement pathway;
e. use of a compound of the present disclosure as described herein, or a pharmaceutically acceptable salt, prodrug, N-oxide, or isolated isomer thereof, optionally in a pharmaceutically acceptable composition, in the manufacture of a medicament for treating or preventing a disorder, including but not limited to the development of fatty liver and conditions stemming from fatty liver, such as nonalcoholic steatohepatitis (NASH), liver inflammation, cirrhosis, liver failure; dermatomyositis; amyotrophic lateral sclerosis; cytokine or inflammatory reactions in response to biotherapeutics (e.g., CAR T-cell therapy); hereditary angioedema (HAE), chronic immune thrombocytopenia (ITP), cold agglutinin disease, cold agglutinin syndrome, warm autoimmune hemolytic anemia, cryoglobulinemia, bullous pemphigoid, common variable immunodeficiency, endotoxemia, sepsis, multiple organ dysfunction syndrome, hemolytic uremic syndrome (HUS), atypical hemolytic uremic syndrome (aHUS), acute kidney injury, kidney transplantation, graft rejection, antibody-mediated rejection, delayed graft function, end-stage renal disease, myasthenia gravis, systemic lupus erythema (SLE), paroxysmal nocturnal hemoglobinuria (PNH), rheumatoid arthritis, multiple sclerosis, age-related macular degeneration (AMD), retinal degeneration, other ophthalmic diseases (e.g., geographic atrophy), a respiratory disease or a cardiovascular disease; a disorder of the central nervous system or peripheral nervous system, ischaemic-reperfusion injury or stroke, traumatic brain injury (TBI) and spinal cord injury (SCI), Alzheimer's disease (AD), multiple sclerosis, neuromyelitis optica (NMO), amyotrophic lateral sclerosis (ALS), Parkinson's disease (PD), Huntington's disease (HD), demyelinating myelinoclastic diseases, demyelinating leukostrophic diseases, and neurological inflammatory disorders;
f. a process for manufacturing a medicament intended for the therapeutic use for treating or preventing a disorder, or generally for treating or preventing disorders mediated by the classical complement pathway, characterized in that a compound of the present disclosure or an embodiment of the active compound is used in the manufacture;
g. a compound of the present disclosure or a salt thereof as described herein in substantially pure form (e.g., at least 90 or 95%);
h. a compound of the present disclosure as described herein, or a pharmaceutically acceptable salt, prodrug, N-oxide, or isolated isomer thereof, optionally in a carrier to form a pharmaceutically acceptable composition, for use in treating a medical disorder which is an inflammatory or immune condition, a disorder mediated by the complement cascade (including a dysfunctional cascade), a disorder or abnormality of a cell that adversely affects the ability of the cell to engage in or respond to normal complement activity, or an undesired complement-mediated response to a medical treatment, such as surgery or other medical procedure or a pharmaceutical or biopharmaceutical drug administration, a blood transfusion, or other allogenic tissue or fluid administration.
i. For each of (a) through (h) above, and otherwise herein, each assembly of moieties and each active compound made therefrom or its use is considered and deemed specifically and individually disclosed, as such depiction is for convenience of space only and not intended to describe a only a genus or even a subgenus for such indication.

### DETAILED DESCRIPTION

### Terminology

Compounds are described using standard nomenclature. Unless defined otherwise, all technical and scientific terms used herein have the same meaning as is commonly understood by one of skill in the art to which the claimed invention belongs.

The compounds in any of the Formulas described herein include enantiomers, mixtures of enantiomers, diastereomers, tautomers, racemates and other isomers, such as rotamers, as if each is specifically described, unless otherwise indicated or otherwise excluded by context.

The terms “a” and “an” do not denote a limitation of quantity, but rather denote the presence of at least one of the referenced item. The term “or” means “and/or”. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of examples, or exemplary language (e.g., “such as”), is intended merely as illustration, and does not pose a limitation on the scope of the invention, which is defined by the claims.

The compound of the present disclosure may form a solvate with solvents (including water). Therefore, in one embodiment, the disclosure includes a solvated form of the active compound. The term “solvate” refers to a molecular complex of a compound of the present disclosure (including a salt thereof) with one or more solvent molecules. Non-limiting examples of solvents are water, ethanol, dimethyl sulfoxide, acetone and other common organic solvents. The term “hydrate” refers to a molecular complex comprising a compound of the disclosure and water. Pharmaceutically acceptable solvates in accordance with the disclosure include those wherein the solvent of crystallization may be isotopically substituted, e.g. D₂O, d₆-acetone, d₆-DMSO. A solvate can be in a liquid or solid form.

A dash (“-”) that is not between two letters or symbols is used to indicate a point of attachment for a substituent. For example, —(C═O)NH₂ is attached through carbon of the keto (C═O) group.

The term “substituted”, as used herein, means that any one or more hydrogens on the designated atom or group is replaced with a moiety selected from the indicated group, provided that the designated atom's normal valence is not exceeded and the resulting compound is stable. For example, when the substituent is oxo (i.e., ═O) then two hydrogens on the atom are replaced. For example, a pyridyl group substituted by oxo is a pyridone. Combinations of substituents and/or variables are permissible only if such combinations result in stable compounds or useful synthetic intermediates.

A stable active compound refers to a compound that can be isolated and can be formulated into a dosage form with a shelf life of at least one month. A stable manufacturing intermediate or precursor to an active compound is stable if it does not degrade within the period needed for reaction or other use. A stable moiety or substituent group is one that does not degrade, react or fall apart within the period necessary for use. Non-limiting examples of unstable moieties are those that combine heteroatoms in an unstable arrangement, as typically known and identifiable to those of skill in the art.

Any suitable group may be present on a “substituted” or “optionally substituted” position that forms a stable molecule and meets the desired purpose of the disclosure and includes, but is not limited to, e.g., halogen (which can independently be F, Cl, Br or I); cyano; hydroxyl; nitro; azido; alkanoyl (such as a C₂-C₆ alkanoyl group); carboxamide; alkyl, carbocycle (e.g., cycloalkyl or cycloalkenyl), alkenyl, alkynyl, alkoxy, aryloxy such as phenoxy; thioalkyl, including those having one or more thioether linkages; alkylsulfinyl; alkylsulfonyl groups, including those having one or more sulfonyl linkages; aryl (e.g., phenyl, biphenyl, naphthyl, or the like, each ring either substituted or unsubstituted); arylalkyl having, for example, 1 to 3 separate or fused rings and from 6 to about 14 or 18 ring carbon atoms, with benzyl being an exemplary arylalkyl group; arylalkoxy, for example, having 1 to 3 separate or fused rings with benzyloxy being an exemplary arylalkoxy group; or a saturated or partially unsaturated heterocycle having 1 to 3 separate or fused rings with one or more N, O or S atoms, or a heteroaryl having 1 to 3 separate or fused rings with one or more N, O or S atoms, e.g., coumarine, quinoline, isoquinoline, quinazoline, pyridine, pyrazole, oxadiazole, triazole, pyrazine, pyrimidine, furan, pyrrole, thienyl, thiazole, triazine, oxazole, isoxazole, imidazole, indole, benzofuran, benzothiazole, tetrahydrofuran, tetrahydropyran, piperidine, morpholine, piperazine, and pyrrolidine. Such groups may be further substituted, e.g. with hydroxy, alkyl, alkoxy, halogen and amino. In certain embodiments “optionally substituted” includes one or more substituents independently selected from halogen, hydroxyl, amino, cyano, —CHO, —COOH, —CONH₂, alkyl including C₁-C₆alkyl, alkenyl including C₂-C₆alkenyl, alkynyl including C₂-C₆alkynyl, —C₁-C₆alkoxy, alkanoyl including C₂-C₆alkanoyl, (mono- and di-C₁-C₆alkylamino)C₀-C₂alkyl, haloalkyl including C₁-C₆haloalkyl, hydoxyC₁-C₆alkyl, ester, carbamate, urea, sulfonamide, —C₁-C₆alkyl(heterocyclo), C₁-C₆alkyl(heteroaryl), —C₁-C₆alkyl(C₃-C₇cycloalkyl), O—C₁-C₆alkyl(C₃-C₇cycloalkyl), B(OH)₂, phosphate, phosphonate and haloalkoxy including C₁-C₆haloalkoxy.

“Alkyl” is a branched or straight chain saturated hydrocarbon group. In one embodiment, the alkyl contains from 1 to about 12 carbon atoms, more generally from 1 to about 6 carbon atoms or from 1 to about 4 carbon atoms. In one embodiment, the alkyl contains from 1 to about 8 carbon atoms. In certain embodiments, the alkyl is C₁-C₂, C₁-C₃, C₁-C₄, C₁-C₆ or C₁-C₆. The specified ranges as used herein indicate an alkyl group having each member of the range described as an independent species. For example, the term C₁-C₆ alkyl as used herein indicates a straight or branched alkyl group having from 1, 2, 3, 4, 5, or 6 carbon atoms and is intended to mean that each of these is described as an independent species. For example, the term C₁-C₄alkyl as used herein indicates a straight or branched alkyl group having from 1, 2, 3, or 4 carbon atoms and is intended to mean that each of these is described as an independent species. When C₀-Cₙ alkyl is used herein in conjunction with another group, for example, (C₃-C₇cycloalkyl)C₀-C₄ alkyl, or —C₀-C₄alkyl(C₃-C₇cycloalkyl), the indicated group, in this case cycloalkyl, is either directly bound by a single covalent bond (C₀alkyl), or attached by an alkyl chain in this case 1, 2, 3, or 4 carbon atoms. Alkyl groups can also be attached via other groups such as heteroatoms as in —O—C₀-C₄alkyl(C₃-C₇cycloalkyl). Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, 2,3-dimethylbutane, and hexyl. Alkyl groups can be optionally substituted independently with one or more substituents described herein.

When a term is used that includes “alk” it should be understood that “cycloalkyl” or “carbocyclic” can be considered part of the definition, unless unambiguously excluded by the context. For example, and without limitation, the terms alkyl, alkenyl, alkynyl, alkoxy, alkanoyl, alkenloxy, haloalkyl, etc., can all be considered to include the cyclic forms of alkyl, unless unambiguously excluded by context.

“Alkenyl” is a branched or straight chain aliphatic hydrocarbon group having one or more carbon-carbon double bonds that may occur at a stable point along the chain. Non-limiting examples are C₂-C₈alkenyl, C₂-C₇alkenyl, C₂-C₆alkenyl, C₂-C₅alkenyl and C₂-C₄alkenyl. The specified ranges as used herein indicate an alkenyl group having each member of the range described as an independent species, as described above for the alkyl moiety. Examples of alkenyl include, but are not limited to, ethenyl and propenyl. Alkenyl groups can be optionally substituted independently with one or more substituents described herein.

“Alkynyl” is a branched or straight chain aliphatic hydrocarbon group having one or more carbon-carbon triple bonds that may occur at any stable point along the chain, for example, C₂-C₈alkynyl or C₂-C₆alkynyl. The specified ranges as used herein indicate an alkynyl group having each member of the range described as an independent species, as described above for the alkyl moiety. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl and 5-hexynyl. Alkynyl groups can be optionally substituted independently with one or more substituents described herein.

“Haloalkyl” indicates both branched and straight-chain alkyl groups substituted with 1 or more halogen atoms, up to the maximum allowable number of halogen atoms. Examples of haloalkyl include, but are not limited to, trifluoromethyl, monofluoromethyl, difluoromethyl, 2-fluoroethyl, and penta-fluoroethyl. Haloalkyl groups can be optionally substituted independently with one or more substituents described herein.

“Halo” or “halogen” indicates independently, any of fluoro, chloro, bromo, or iodo.

“Aryl” indicates an aromatic group containing only carbon in the aromatic ring or rings. In one embodiment, the aryl group contains 1 to 3 separate or fused rings and is 6 to 14 or 18 ring atoms, without heteroatoms as ring members. When indicated, such aryl groups may be further substituted with carbon or non-carbon atoms or groups. Such substitution may include fusion to a 4 to 7 or a 5 to 7-membered saturated or partially unsaturated cyclic group that optionally contains 1, 2 or 3 heteroatoms independently selected from N, O, B, P, Si and S, to form, for example, a 3,4-methylenedioxyphenyl group. Aryl groups include, for example, phenyl and naphthyl, including 1-naphthyl and 2-naphthyl. In one embodiment, aryl groups are pendant. An example of a pendant ring is a phenyl group substituted with a phenyl group. Aryl groups can be optionally substituted independently with one or more substituents described herein.

“Heterocycle” refers to saturated and partially saturated heteroatom-containing ring radicals, where the heteroatoms may be selected from N, S, and O. The term “heterocycle” includes monocyclic 3-12 membered rings, as well as bicyclic 5-16 membered ring systems (which can include fused, bridged, or spiro, bicyclic ring systems), and excludes rings containing —O—O—. —O—S—, or —S—S— portions. Examples of saturated heterocycle groups include saturated 4- to 7-membered monocyclic groups containing 1 to 4 nitrogen atoms [e.g., pyrrolidinyl, imidazolidinyl, piperidinyl, pyrrolinyl, azetidinyl, piperazinyl, and pyrazolidinyl]; saturated 4 to 6-membered monocyclic groups containing 1 to 2 oxygen atoms and 1 to 3 nitrogen atoms [e.g., morpholinyl]; saturated 3 to 6-membered heteromonocyclic group containing 1 to 2 sulfur atoms and 1 to 3 nitrogen atoms [e.g., thiazolidinyl]. Examples of partially saturated heterocycle radicals include but are not limited to, dihydrothienyl, dihydropyranyl, dihydrofuryl, and dihydrothiazolyl. Examples of partially saturated and saturated heterocycle groups include, but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-IH-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3-dihydro-IH-Iλ′-benzo[d]isothiazol-6-yl, dihydropyranyl, dihydrofuryl and dihydrothiazolyl. “Bicyclic heterocycle” includes groups wherein the heterocyclic radical is fused with an aryl radical wherein the point of attachment is the heterocycle ring. “Bicyclic heterocycle” also includes heterocyclic radicals that are fused with a carbocycle radical. For example, partially unsaturated condensed heterocyclic group containing 1 to 5 nitrogen atoms, for example, indoline, isoindoline, partially unsaturated condensed heterocyclic group containing 1 to 2 oxygen atoms and 1 to 3 nitrogen atoms, partially unsaturated condensed heterocyclic group containing 1 to 2 sulfur atoms and 1 to 3 nitrogen atoms, and saturated condensed heterocyclic group containing 1 to 2 oxygen or sulfur atoms are all encompassed. Heterocycle groups can be optionally substituted independently with one or more substituents described herein.

Non-limiting examples of bicyclic heterocycles include:

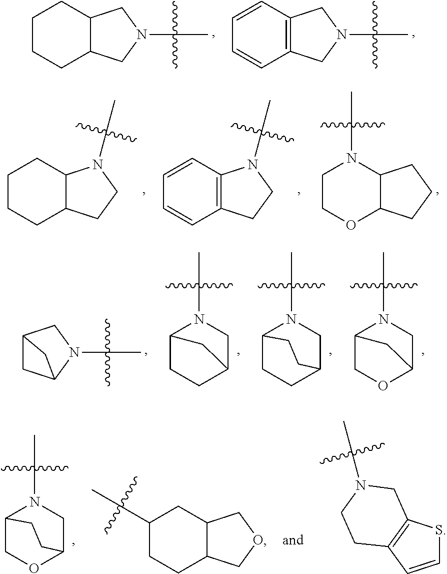

Unless otherwise drawn or clear from the context, the term “bicyclic heterocycle” includes cis and trans diastereomers. Non-limiting examples of chiral bicyclic heterocycles include:

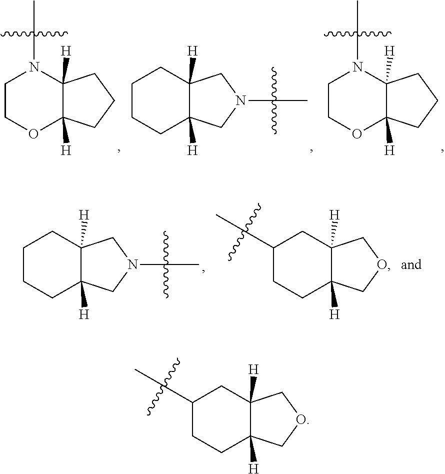

“Carbocycle” refers to a non-aromatic monocyclic or polycyclic (e.g., bicyclic or tricyclic) in which all ring atoms are carbon atoms. Carbocycle groups include saturated groups (i.e., cycloalkyl) and unsaturated groups (e.g., cycloalkenyl, which includes one or more double bonds and no triple bonds and cycloalkynyl, which includes at least one triple bond). In some embodiments, “carbocycle” is cycloalkyl. In some embodiments, “carbocycle” is cycloalkenyl (e.g., cyclopentenyl, cyclohexenyl, cycloheptenyl, or cyclooctenyl). In some embodiments, “carbocycle” includes 3 to 13 carbon atoms. In some embodiments, “carbocycle” is a tricyclic cycloalkenyl group (e.g., fluorenyl). In some embodiments, “carbocycle” (e.g., cycloalkyl or cycloalkenyl) is optionally substituted with one or more substituents described herein.

“Heteroaryl” refers to a stable monocyclic, bicyclic, or multicyclic aromatic ring which contains from 1 to 3, or in some embodiments from 1, 2, or 3 heteroatoms selected from N, O, S, B, and P (and typically selected from N, O, and S) with remaining ring atoms being carbon, or a stable bicyclic or tricyclic system containing at least one 5, 6, or 7 membered aromatic ring which contains from 1 to 3, or in some embodiments from 1 to 2, heteroatoms selected from N, O, S, B or P with remaining ring atoms being carbon. In one embodiment, the only heteroatom is nitrogen. In one embodiment, the only heteroatom is oxygen. In one embodiment, the only heteroatom is sulfur. Monocyclic heteroaryl groups typically have from 5 or 6 ring atoms. In some embodiments, bicyclic heteroaryl groups are 8- to 10-membered heteroaryl groups, that is, groups containing 8 or 10 ring atoms in which one 5, 6, or 7 member aromatic ring is fused to a second aromatic or non-aromatic ring wherein the point of attachment is the aromatic ring. When the total number of S and O atoms in the heteroaryl group exceeds 1, these heteroatoms are not adjacent to one another. In one embodiment, the total number of S and O atoms in the heteroaryl group is not more than 2. In another embodiment, the total number of S and O atoms in the aromatic heterocycle is not more than 1. Examples of heteroaryl groups include, but are not limited to, pyridinyl (including, for example, 2-hydroxypyridinyl), imidazolyl, imidazopyridinyl, pyrimidinyl (including, for example, 4-hydroxypyrimidinyl), pyrazolyl, triazolyl, pyrazinyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, triazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, tetrahydrofuranyl, and furopyridinyl. Heteroaryl groups can be optionally substituted independently with one or more substituents described herein.

A “dosage form” means a unit of administration of an active agent. Examples of dosage forms include tablets, capsules, injections, suspensions, liquids, emulsions, implants, particles, spheres, creams, ointments, suppositories, inhalable forms, transdermal forms, buccal, sublingual, topical, gel, mucosal, and the like. A “dosage form” can also include an implant, for example an optical implant.

“Pharmaceutical compositions” are compositions comprising at least one active agent, and at least one other substance, such as a pharmaceutically acceptable carrier. “Pharmaceutical combinations” are combinations of at least two active agents which may be combined in a single dosage form or provided together in separate dosage forms with instructions that the active agents are to be used together to treat any disorder described herein.

A “pharmaceutically acceptable salt” is a derivative of the disclosed compound in which the parent compound is modified by making inorganic and organic, pharmaceutically acceptable, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. Pharmaceutically acceptable salts include salts which are acceptable for human consumption, and the quaternary ammonium salts of the parent compound formed, for example, from inorganic or organic acids. Examples of such salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)₁₋₄—COOH, and the like, or using a different acid that produces the same counterion. Lists of additional suitable salts may be found, e.g., in Remington's Pharmaceutical Sciences, 17th ed., Mack Publishing Company, Easton, Pa., p. 1418 (1985).

The term “carrier” applied to pharmaceutical compositions/combinations according to the disclosure refers to a diluent, excipient, or vehicle with which an active compound is provided.

A “pharmaceutically acceptable excipient” or “pharmaceutically acceptable carrier” may be used interchangeably and mean an excipient that is useful in preparing a pharmaceutical composition/combination that is generally safe, acceptable for human consumption, and neither biologically nor otherwise inappropriate for administration to a host, typically a human. In one embodiment, an excipient is used that is acceptable for veterinary use. In one embodiment, an excipient is used that is acceptable for mammalian, particularly human, use.

A “patient” or “host” or “subject” is a human or non-human animal in need of treatment or prevention of any of the disorders as specifically described herein, including but not limited to by modulation of the classical complement pathway or with a condition that is treatable with one of the compounds described herein. Typically, the host is a human. A “patient” or “host” or “subject” also refers to for example, a mammal, primate (e.g., human), cows, sheep, goat, horse, dog, cat, rabbit, rat, mice, bird, and the like.

A “prodrug” refers to a compound which when administered to a host in vivo is converted into a parent drug. The term “parent drug” means any of the presently described chemical compounds herein. Prodrugs can be used to achieve any desired effect, including to enhance properties of the parent drug or to improve the pharmaceutic or pharmacokinetic properties of the parent, including to increase the half-life of the drug in vivo. Prodrug strategies provide choices in modulating the conditions for in vivo generation of the parent drug. Non-limiting examples of prodrug strategies include covalent attachment of removable groups, or removable portions of groups, for example, but not limited to acylation, phosphorylation, phosphonylation, phosphoramidate derivatives, amidation, reduction, oxidation, esterification, alkylation, other carboxy derivatives, sulfoxy or sulfone derivatives, carbonylation or anhydride, among others.

“Providing a compound with at least one additional active agent,” for example, in one embodiment can mean that the compound and the additional active agent(s) are provided simultaneously in a single dosage form, provided concomitantly in separate dosage forms, or provided in separate dosage forms for administration. In one embodiment, the compound administrations are separated by some amount of time that is within the time in which both the compound and the at least one additional active agent are within the blood stream of a patient. In certain embodiments, the compound and the additional active agent need not be prescribed for a patient by the same medical care worker. In certain embodiments, the additional active agent or agents need not require a prescription. Administration of the compound or the at least one additional active agent can occur via any appropriate route, for example, oral tablets, oral capsules, oral liquids, inhalation, injection, suppositories, parenteral, sublingual, buccal, intravenous, intraaortal, transdermal, polymeric controlled delivery, non-polymeric controlled delivery, nano or microparticles, liposomes, and/or topical contact. In one embodiment, the instructions for administration in a form of combination therapy is provided in the drug labeling.

A “therapeutically effective amount” of a pharmaceutical composition/combination of this disclosure means an amount effective, when administered to a host, to provide a therapeutic benefit, such as an amelioration of symptoms or reduction ordimunition of the disease itself. In one embodiment, a therapeutically effective amount is an amount sufficient to prevent a significant increase, or will significantly reduce, the detectable level of hemolysis in the patient's blood, serum, or tissues.

### N-Oxides

In certain embodiments, any of the active compounds can be provided in its N-oxide form to a patient in need thereof. In one embodiment, an N-oxide of an active compound or a precursor of the active compound is used in a manufacturing scheme. In yet another embodiment, the N-oxide is a metabolite of administration of one of the active compounds herein, and may have independent activity. The N-oxide can be formed by treating the compound of interest with an oxidizing agent, for example, a suitable peroxyacid or peroxide, to generate an N-oxide compound. For example, a heteroaryl group, for example a pyridyl group, can be treated with an oxidizing agent such as sodium percarbonate in the presence of a rhenium-based catalyst under mild reaction conditions to generate an N-oxide compound. A person skilled in the art will understand that appropriate protecting groups may be necessary to carry out the chemistry. See Jain, S. L. et al., “Rhenium-Catalyzed Highly Efficient Oxidations of Tertiary Nitrogen Compounds to N-Oxides Using Sodium Percarbonate as Oxygen Source, Synlett, 2261-2663, 2006.

In other aspects of the present disclosure, any of the active compounds with a sulfur can be provided in its sulfoxide or sulfone form to a patient in need thereof. In a different embodiment, a sulfoxide or sulfone of one of the active compounds or a precursor of the active compound is used in a manufacturing scheme. A sulfur atom in a selected compound as described herein can be oxidized to form a sulfoxide

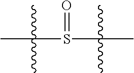 [SMILES: *S(*)=O]

or a sulfone

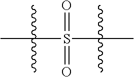 [SMILES: *S(*)(=O)=O]

using known methods. For example, the compound 1,3,5-triazo-2,4,6-triphosphorine-2,2,4,4,6,6-tetrachloride (TAPC) is an efficient promoter for the oxidation of sulfides to sulfoxides. See, Bahrami, M. et al., “TAPC-Promoted Oxidation of sulfides and Deoxygenation of Sulfoxides”, J. Org. Chem., 75, 6208-6213 (2010). Oxidation of sulfides with 30% hydrogen peroxide catalyzed by tantalum carbide provides sulfoxides in high yields, see Kirihara, A., et al., “Tantalum Carbide or Niobium Carbide Catalyzed Oxidation of Sulfides with Hydrogen Peroxide: Highly Efficient and Chemoselective Syntheses of Sulfoxides and Sulfones”, Synlett, 1557-1561 (2010). Sulfides can be oxidized to sulfones using, for example, niobium carbide as the catalyst, see Kirihara, A., et al., “Tantalum Cardide or Niobium Carbide Catalyzed Oxidation of Sulfides with Hydrogen Peroxide: Highly Efficient and Chemoselective Syntheses of Sulfoxides and Sulfones”, Synlett, 1557-1561 (2010). Urea-hydrogen peroxide adduct is a stable inexpensive and easily handled reagent for the oxidation of sulfides to sulfones, see Varma, R. S. and Naicker, K. P., “The Urea-Hydrogen Peroxide Complex: Solid-State Oxidative Protocols for Hydroxylated Aldehydes and Ketones (Dakin Reaction), Nitriles, Sulfides, and Nitrogen Heterocycles”, Org. Lett., 1, 189-191 (1999). One skilled in the art will appreciate that other heteroatoms, such as nitrogen, may need to be protected and then deprotected while carrying out the oxidation of a sulfur atom to produce the desired compound.

### Embodiments of “Alkyl”

In certain embodiments, “alkyl” is a C₁-C₁₀alkyl, C₁-C₉alkyl, C₁-C₈alkyl, C₁-C₇alkyl, C₁-C₆alkyl, C₁-C₅alkyl, C₁-C₄alkyl, C₁-C₃alkyl, or C₁-C₂alkyl.

In certain embodiments, “alkyl” has one carbon.

In certain embodiments, “alkyl” has two carbons.

In certain embodiments, “alkyl” has three carbons.

In certain embodiments, “alkyl” has four carbons.

In certain embodiments, “alkyl” has five carbons.

In certain embodiments, “alkyl” has six carbons.

Non-limiting examples of “alkyl” include: methyl, ethyl, propyl, butyl, pentyl, and hexyl.

Additional non-limiting examples of “alkyl” include: isopropyl, isobutyl, isopentyl, and isohexyl.

Additional non-limiting examples of “alkyl” include: sec-butyl, sec-pentyl, and sec-hexyl.

Additional non-limiting examples of “alkyl” include: tert-butyl, tert-pentyl, and tert-hexyl.

Additional non-limiting examples of “alkyl” include: neopentyl, 3-pentyl, and active pentyl.

### Embodiments of “Haloalkyl”

In certain embodiments, “haloalkyl” is a C₁-C₁₀haloalkyl, C₁-C₉haloalkyl, C₁-C₈haloalkyl, C₁-C₇haloalkyl, C₁-C₆haloalkyl, C₁-C₅haloalkyl, C₁-C₄haloalkyl, C₁-C₃haloalkyl, and C₁-C₂haloalkyl.

In certain embodiments, “haloalkyl” has one carbon.

In certain embodiments, “haloalkyl” has one carbon and one halogen.

In certain embodiments, “haloalkyl” has one carbon and two halogens.

In certain embodiments, “haloalkyl” has one carbon and three halogens.

In certain embodiments, “haloalkyl” has two carbons.

In certain embodiments, “haloalkyl” has three carbons.

In certain embodiments, “haloalkyl” has four carbons.

In certain embodiments, “haloalkyl” has five carbons.

In certain embodiments, “haloalkyl” has six carbons.

Non-limiting examples of “haloalkyl” include:

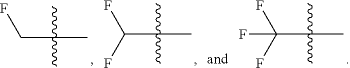

Additional non-limiting examples of “haloalkyl” include:

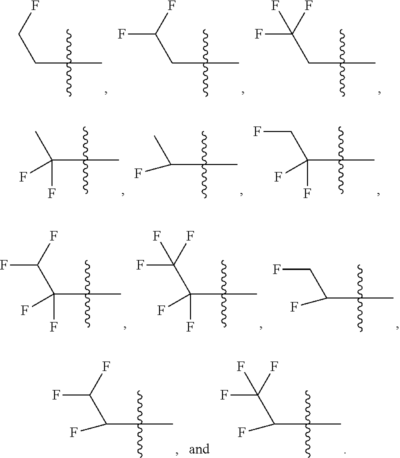

Additional non-limiting examples of “haloalkyl” include:

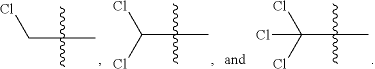

Additional non-limiting examples of “haloalkyl” include:

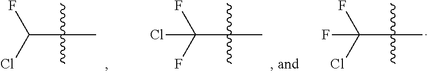

### Embodiments of “Aryl”

In certain embodiments, “aryl” is a 6 carbon aromatic group (phenyl)

In certain embodiments, “aryl” is a 10 carbon aromatic group (napthyl)

In certain embodiments, “aryl” is “substituted aryl”.

### Embodiments of “Heteroaryl”

In certain embodiments, “heteroaryl” is a 5 membered aromatic group containing 1, 2, or 3, nitrogen atoms.

Non-limiting examples of 5 membered “heteroaryl” groups include pyrrole, furan, thiophene, pyrazole, imidazole, triazole, isoxazole, oxazole, oxadiazole, oxatriazole, isothiazole, thiazole, thiadiazole, and thiatriazole.

Additional non-limiting examples of 5 membered “heteroaryl” groups include:

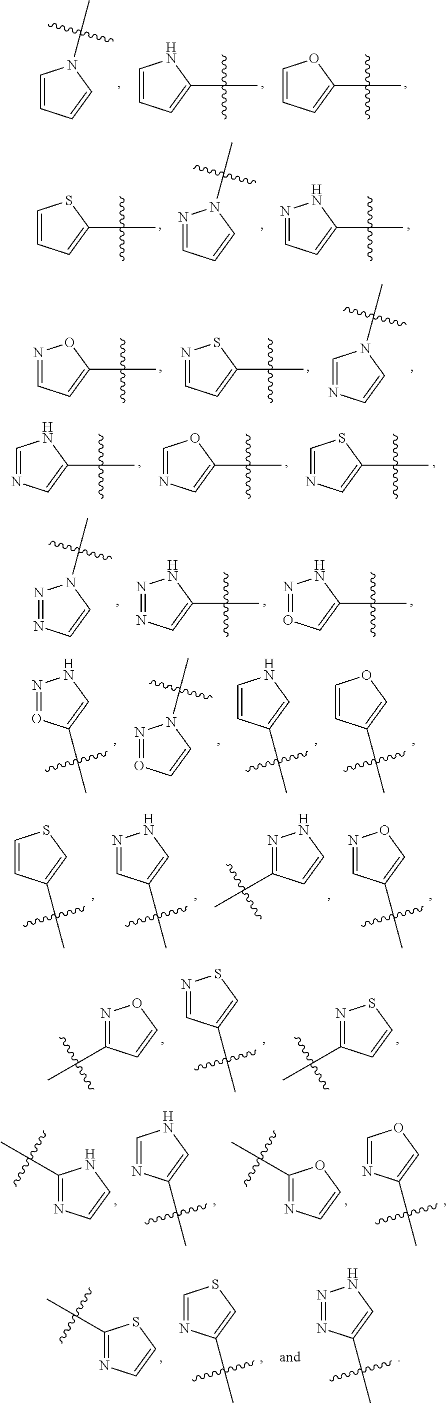

In certain embodiments, “heteroaryl” is a 6 membered aromatic group containing 1, 2, or 3 nitrogen atoms (i.e. pyridinyl, pyridazinyl, triazinyl, pyrimidinyl, and pyrazinyl).

Non-limiting examples of 6-membered “heteroaryl” groups with 1 or 2 nitrogen atoms include:

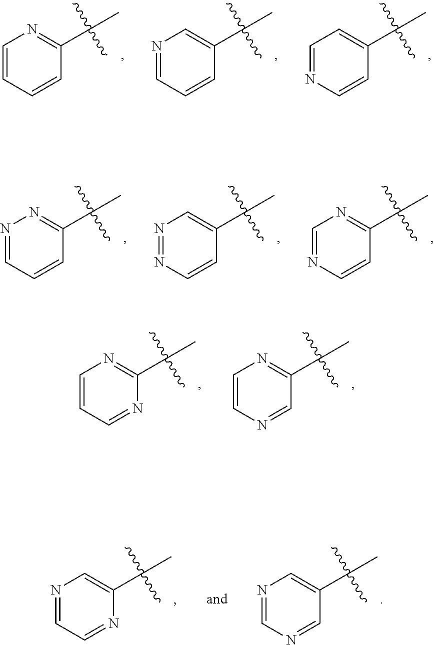

In certain embodiments, “heteroaryl” is a 9 membered bicyclic aromatic group containing 1 or 2 atoms selected from nitrogen, oxygen, and sulfur.

Non-limiting examples of “heteroaryl” groups that are bicyclic include indole, benzofuran, isoindole, indazole, benzimidazole, azaindole, azaindazole, purine, isobenzofuran, benzothiophene, benzoisoxazole, benzoisothiazole, benzooxazole, and benzothiazole.

Additional non-limiting examples of “heteroaryl” groups that are bicyclic include:

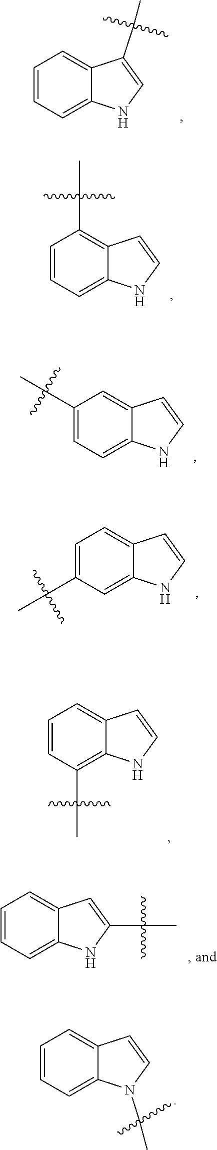

Additional non-limiting examples of “heteroaryl” groups that are bicyclic include:

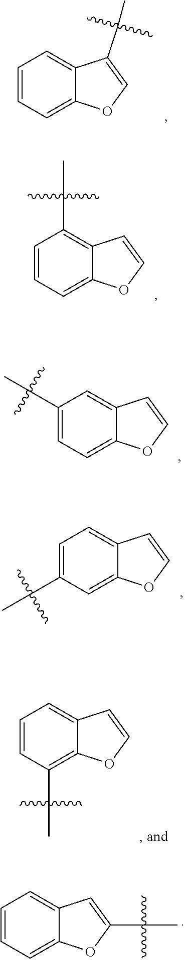 [SMILES: *c1coc2ccccc12 ; *c1cccc2occc12 ; *c1ccc2occc2c1 ; *c1ccc2ccoc2c1 ; *c1cccc2ccoc12 ; *c1cc2ccccc2o1]

Additional non-limiting examples of “heteroaryl” groups that are bicyclic include:

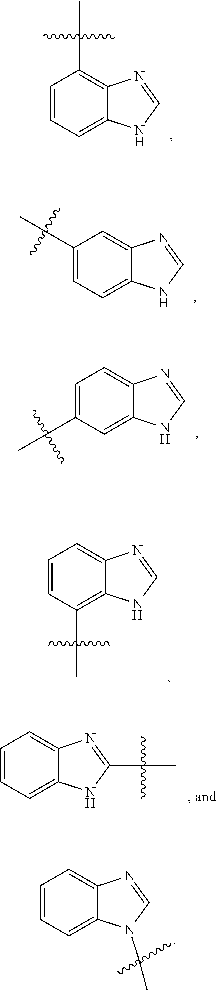 [SMILES: *c1cccc2[nH]cnc12 ; *c1ccc2[nH]cnc2c1 ; *c1ccc2nc[nH]c2c1 ; *c1cccc2nc[nH]c12 ; *c1nc2ccccc2[nH]1 ; *n1cnc2ccccc21]

In one embodiment “heteroaryl” is a 10 membered bicyclic aromatic group containing 1 or 2 atoms selected from nitrogen, oxygen, and sulfur.

Non-limiting examples of “heteroaryl” groups that are bicyclic include quinoline, isoquinoline, quinoxaline, phthalazine, quinazoline, cinnoline, and naphthyridine.

Additional non-limiting examples of “heteroaryl” groups that are bicyclic include:

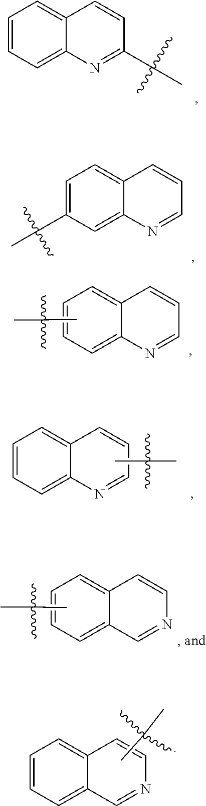

In an alternative embodiment, heteroaryl is tetrazole.

### Embodiments of “Carbocycle”

In certain embodiments, “carbocycle” is a saturated or an unsaturated, non-aromatic cyclic group containing only carbon atoms as the ring atoms, e.g., C₃-C₈carbocycle, C₃-C₇ carbocycle, C₃-C₆ carbocycle, C₃-C₅ carbocycle, C₃-C₄carbocycle, C₄-C₈carbocycle, C₅-C₈ carbocycle, or C₆-C₈ carbocycle.

In certain embodiments, “carbocycle” has three carbons.

In certain embodiments, “carbocycle” has four carbons.

In certain embodiments, “carbocycle” has five carbons.

In certain embodiments, “carbocycle” has six carbons.

In certain embodiments, “carbocycle” has seven carbons.

In certain embodiments, “carbocycle” has eight carbons.

In certain embodiments, “carbocycle” has nine carbons.

In certain embodiments, “carbocycle” has ten carbons.

In certain embodiments, “carbocycle” has eleven carbons.

In certain embodiments, “carbocycle” has twelve carbons.

In certain embodiments, “carbocycle” has thirteen carbons.

In certain embodiments, “carbocycle” is a saturated cyclic group, i.e., a “cycloalkyl” group.

In certain embodiments, “carbocycle” is an unsaturated, non-aromatic cyclic group, i.e., a “cycloalkenyl” group.

### Embodiments of “Cycloalkyl”

In certain embodiments, “cycloalkyl” is a C₃-C₈cycloalkyl, C₃-C₇cycloalkyl, C₃-C₆cycloalkyl, C₃-C₅cycloalkyl, C₃-C₄cycloalkyl, C₄-C₈cycloalkyl, C₅-C₈cycloalkyl, or C₆-C₈cycloalkyl.

In certain embodiments, “cycloalkyl” has three carbons.

In certain embodiments, “cycloalkyl” has four carbons.

In certain embodiments, “cycloalkyl” has five carbons.

In certain embodiments, “cycloalkyl” has six carbons.

In certain embodiments, “cycloalkyl” has seven carbons.

In certain embodiments, “cycloalkyl” has eight carbons.

In certain embodiments, “cycloalkyl” has nine carbons.

In certain embodiments, “cycloalkyl” has ten carbons.

Non-limiting examples of “cycloalkyl” include: cyclopropyl, cyclobutyl, cyclopentyl, cyclohexyl, cycloheptyl, cyclooctyl, and cyclodecyl.

### Embodiments of “Cycloalkenyl”

In certain embodiments, “cycloalkyl” is a C₄-C₈cycloalkenyl, C₄-C₇cycloalkenyl, C₄-C₆cycloalkenyl, C₄-C₅cycloalkenyl, C₄-C₉cycloalkenyl, C₄-C₁₀cycloalkenyl, C₄-C₁₁cycloalkenyl, C₄-C₁₂cycloalkenyl, C₄-C₁₃cycloalkenyl, C₅-C₈cycloalkenyl, or C₆-C₈cycloalkenyl.

In certain embodiments, “cycloalkenyl” has four carbons.

In certain embodiments, “cycloalkenyl” has five carbons.

In certain embodiments, “cycloalkenyl” has six carbons.

In certain embodiments, “cycloalkenyl” has seven carbons.

In certain embodiments, “cycloalkenyl” has eight carbons.

In certain embodiments, “cycloalkenyl” has nine carbons.

In certain embodiments, “cycloalkenyl” has ten carbons.

In certain embodiments, “cycloalkenyl” has eleven carbons.

In certain embodiments, “cycloalkenyl” has twelve carbons.

In certain embodiments, “cycloalkenyl” has thirteen carbons.

In certain embodiments, “cycloalkenyl” includes one double bond.

In certain embodiments, “cycloalkenyl” includes two or more double bonds.

In certain embodiments, “cycloalkenyl” is a bicyclic group.

In certain embodiments, “cycloalkenyl” is a tricyclic group.

Non-limiting examples of “cycloalkenyl” include: cyclobutenyl, cyclopentenyl, cyclohexenyl, cycloheptenyl, cyclooctenyl, and cyclodecenyl.

A non-limiting example of tricyclic “alkenyl” is fluorenyl.

### Embodiments of “Heterocycle”

In one embodiment, “heterocycle” refers to a saturated or unsaturated, non-aromatic cyclic ring with one nitrogen and 3, 4, 5, 6, 7, or 8 carbon atoms.

In one embodiment, “heterocycle” refers to a saturated or unsaturated, non-aromatic cyclic ring with one nitrogen and one oxygen and 3, 4, 5, 6, 7, or 8 carbon atoms.

In one embodiment, “heterocycle” refers to a saturated or unsaturated, non-aromatic cyclic ring with two nitrogens and 3, 4, 5, 6, 7, or 8 carbon atoms.

In one embodiment, “heterocycle” refers to a saturated or unsaturated, non-aromatic cyclic ring with one oxygen and 3, 4, 5, 6, 7, or 8 carbon atoms.

In one embodiment, “heterocycle” refers to a saturated or unsaturated, non-aromatic cyclic ring with one sulfur and 3, 4, 5, 6, 7, or 8 carbon atoms.

Non-limiting examples of “heterocycle” include aziridine, oxirane, thiirane, azetidine, 1,3-diazetidine, oxetane, and thietane.

Additional non-limiting examples of “heterocycle” include pyrrolidine, 3-pyrroline, 2-pyrroline, pyrazolidine, and imidazolidine.

Additional non-limiting examples of “heterocycle” include tetrahydrofuran, 1,3-dioxolane, tetrahydrothiophene, 1,2-oxathiolane, and 1,3-oxathiolane.

Additional non-limiting examples of “heterocycle” include piperidine, piperazine, tetrahydropyran, 1,4-dioxane, thiane, 1,3-dithiane, 1,4-dithiane, morpholine, and thiomorpholine.

Additional non-limiting examples of “heterocycle” include dihydrooxadiazole and dihydropyrimidine.

Non-limiting examples of “heterocycle” also include

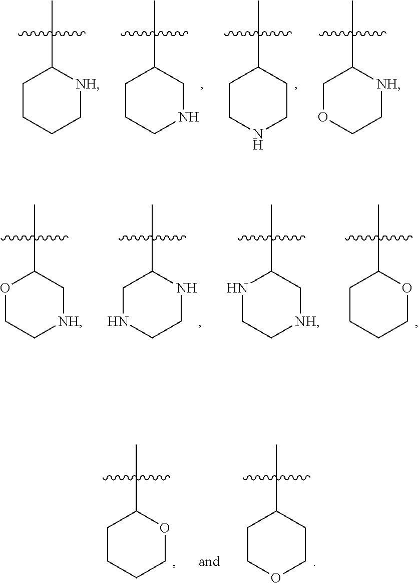

Additional non-limiting examples of “heterocycle” include:

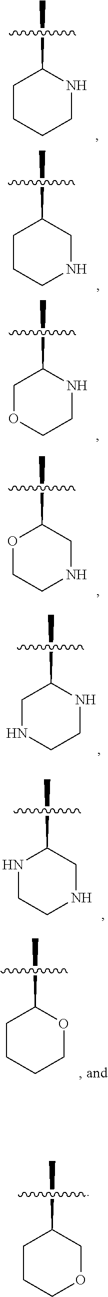

Additional non-limiting examples of “heterocycle” include:

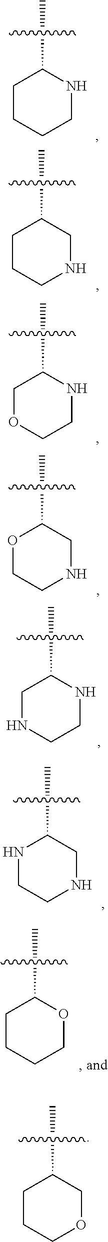

Non-limiting examples of “heterocycle” also include:

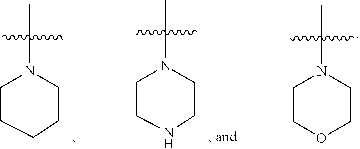 [SMILES: *N1CCCCC1 ; *N1CCNCC1 ; *N1CCOCC1]

Non-limiting examples of “heterocycle” also include:

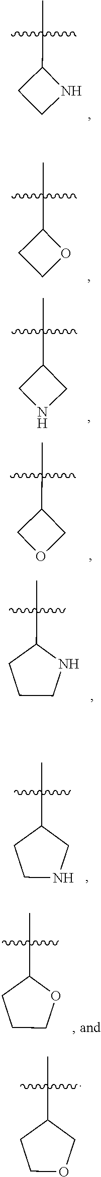

Additional non-limiting examples of “heterocycle” include:

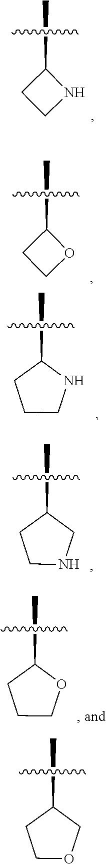

Additional non-limiting examples of “heterocycle” include:

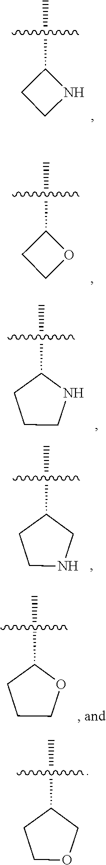

### Embodiments of “Sugar”

In some embodiments, “sugar” refers to a compound of formula C₃H₅O₃, C₄H₇O₄, C₅H₉O₅, C₆H₁₁O₆, C₇H₁₃O₇, or C₈H₁₅O₈.

Non-limiting examples of sugar include

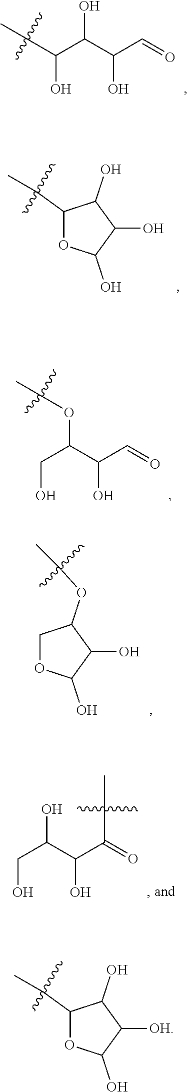 [SMILES: *C(O)C(O)C(O)C=O ; *C1OC(O)C(O)C1O ; *OC(CO)C(O)C=O ; *OC1COC(O)C1O ; *C(=O)C(O)C(O)CO ; *C1OC(O)C(O)C1O]

### ADDITIONAL EMBODIMENTS OF THE PRESENT DISCLOSURE

In some embodiments,

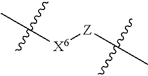

is selected from:

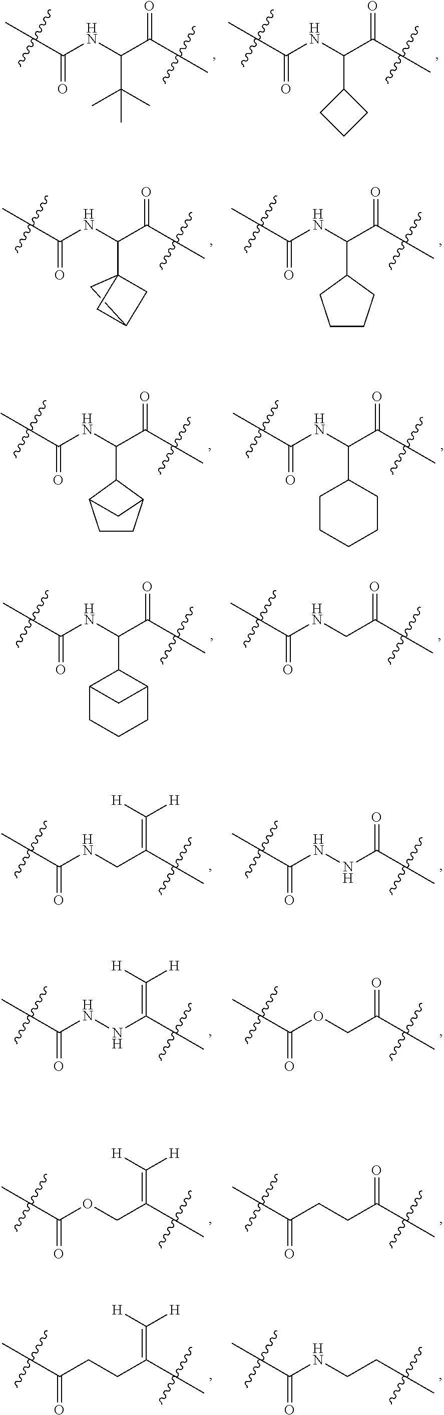

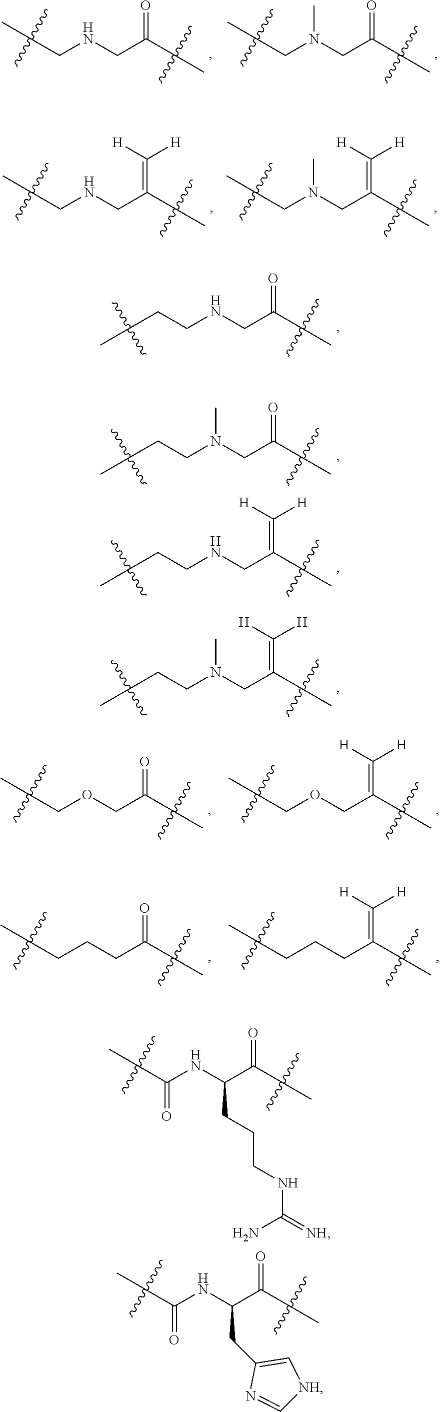

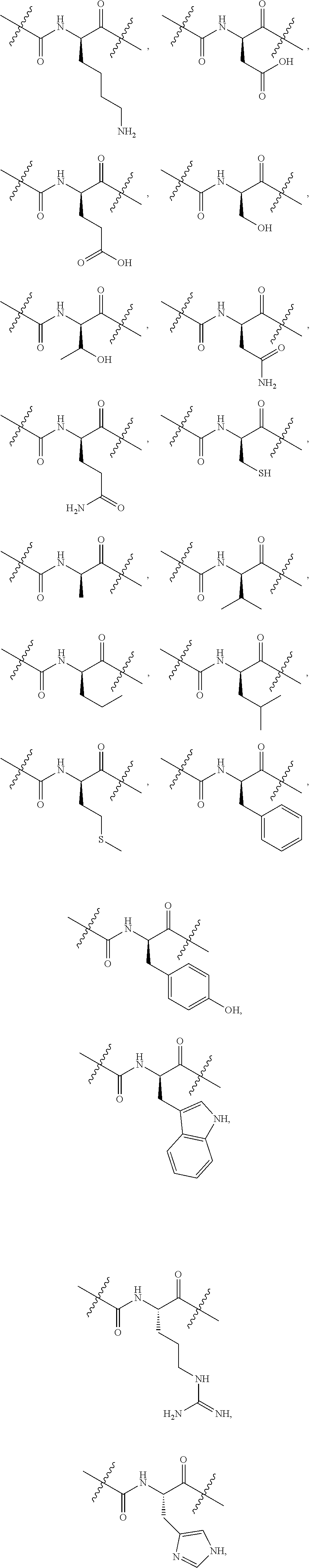

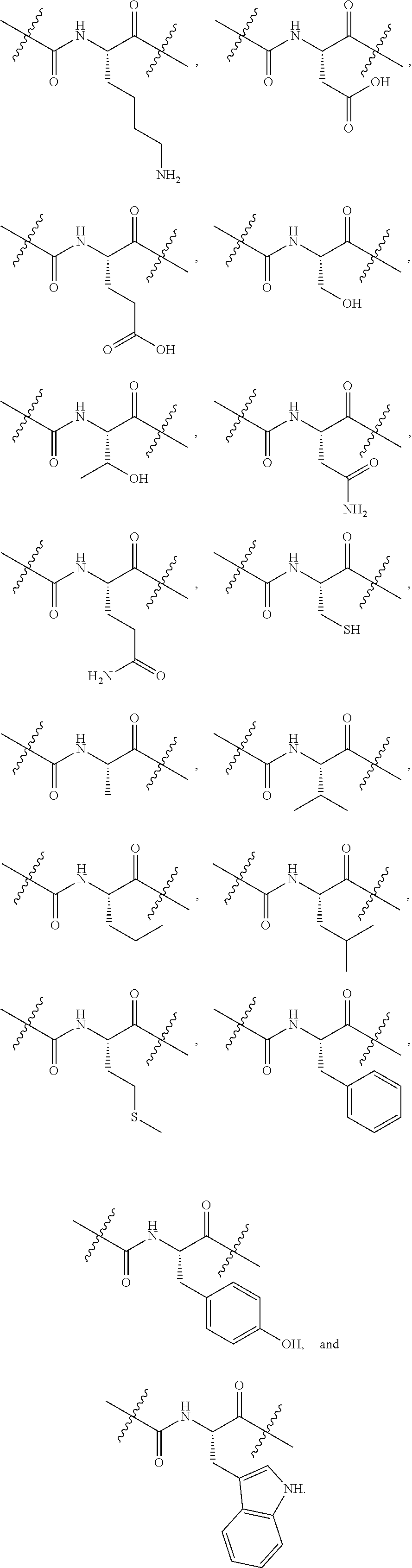

In some embodiments,

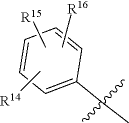

is selected from:

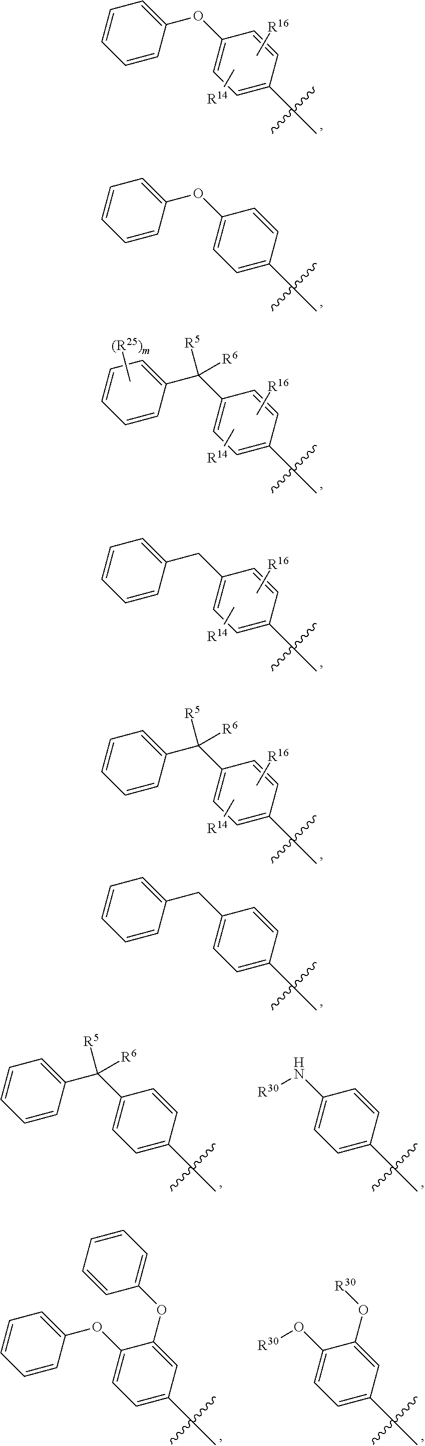

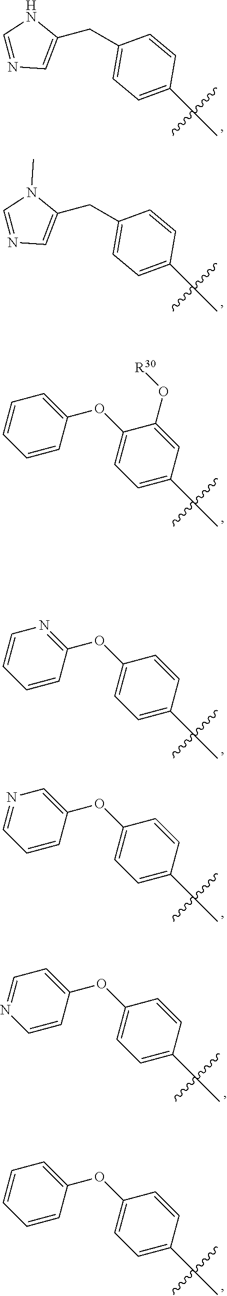

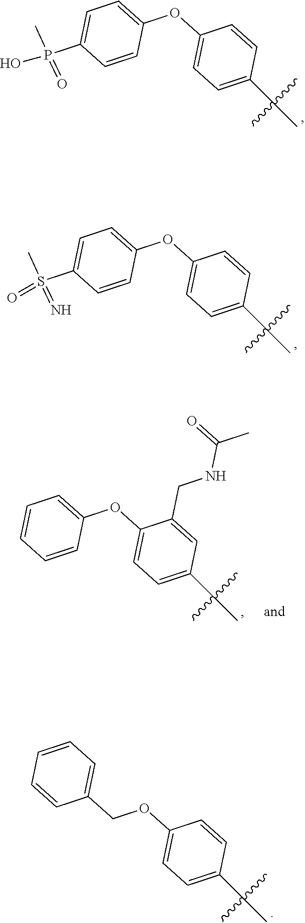 [SMILES: *c1ccc(Oc2ccc(P(C)(=O)O)cc2)cc1 ; *c1ccc(Oc2ccc(S(C)(=N)=O)cc2)cc1 ; *c1ccc(Oc2ccccc2)c(CNC(C)=O)c1 ; *c1ccc(OCc2ccccc2)cc1]

In some embodiments, R²² is selected from

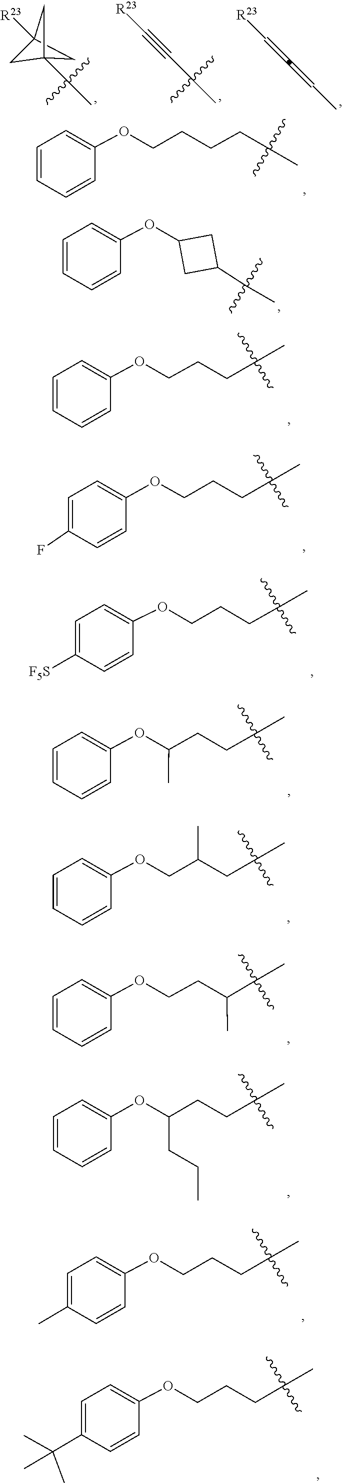

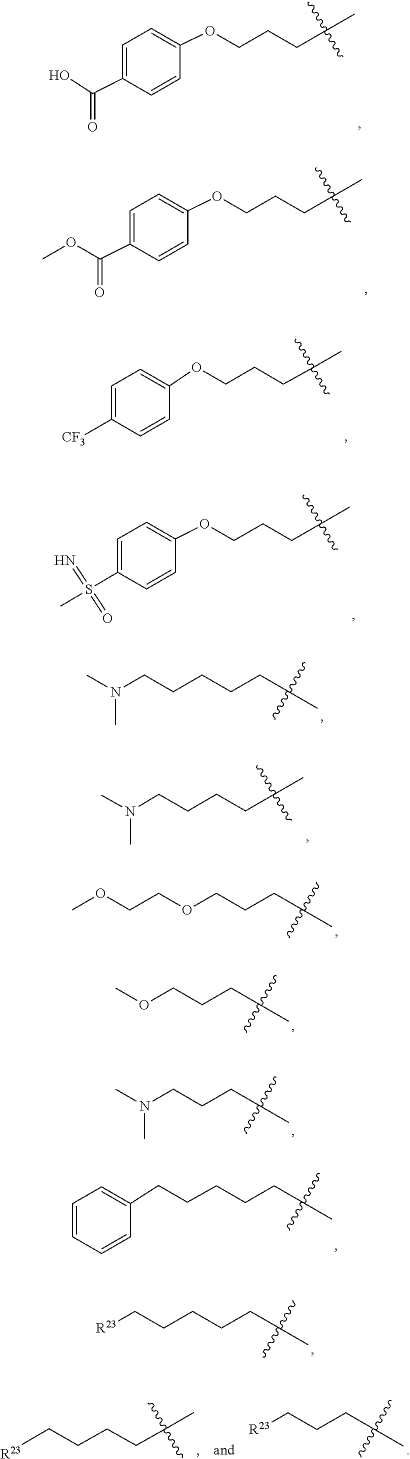

In some embodiments, R²² is selected from

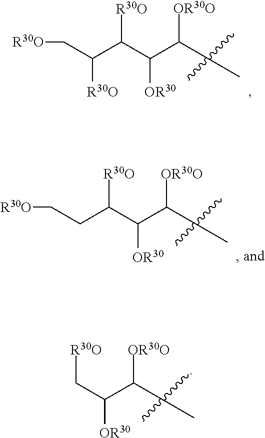

In some embodiments,

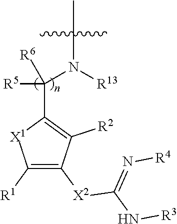

is selected from

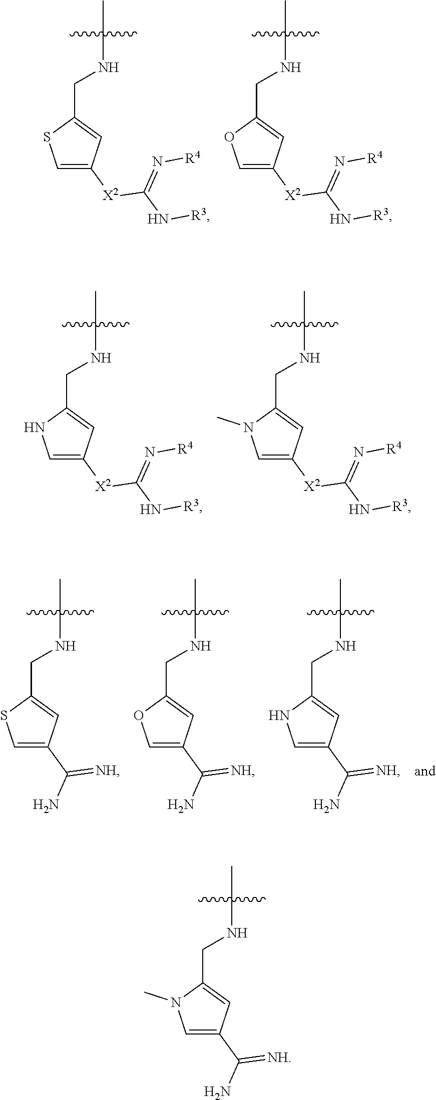

In some embodiments,

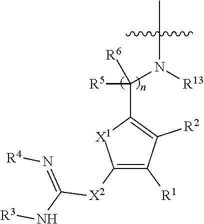

is selected from

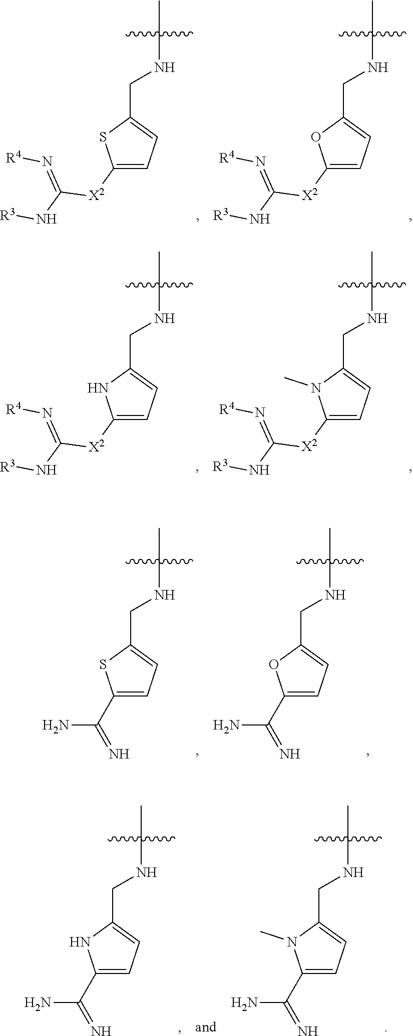

In some embodiments,

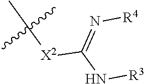

is selected from

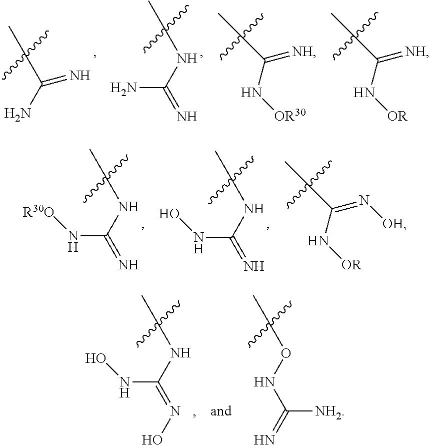

In some embodiments,

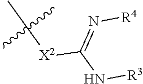

is selected from

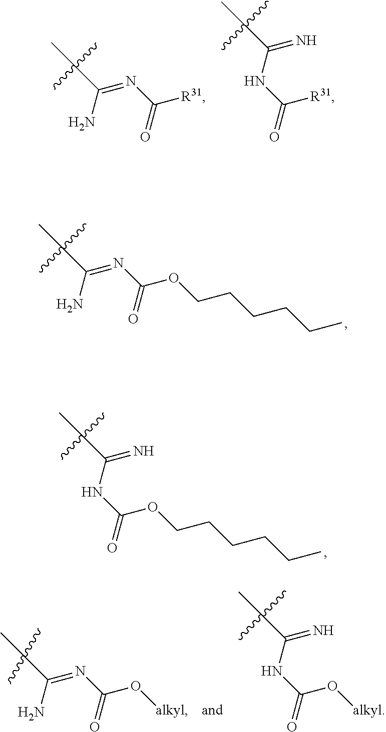

In some embodiments,

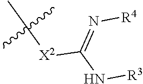

is selected from

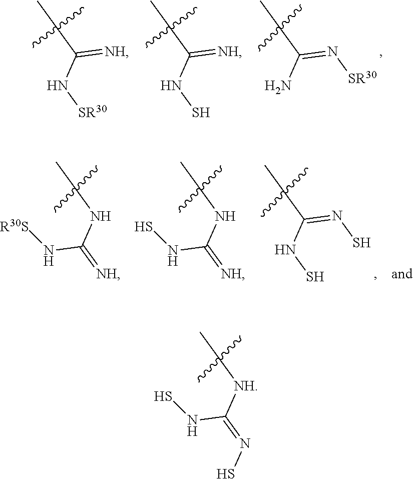

In some embodiments,

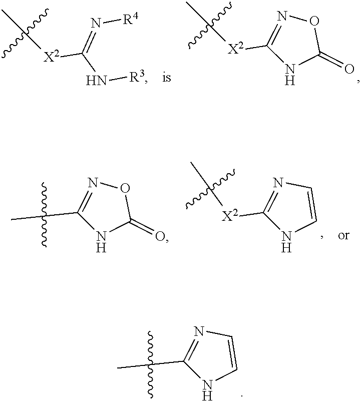

In some embodiments,

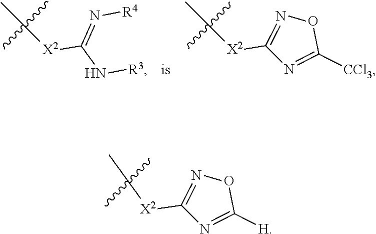

In some embodiments, R²⁶ is selected from

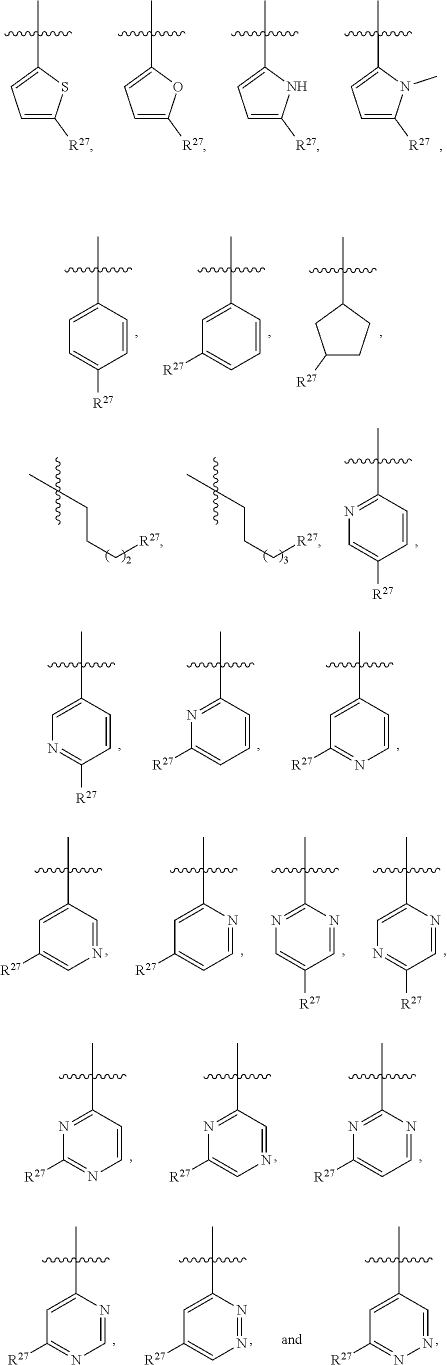

In some embodiments, R²⁷ is selected from

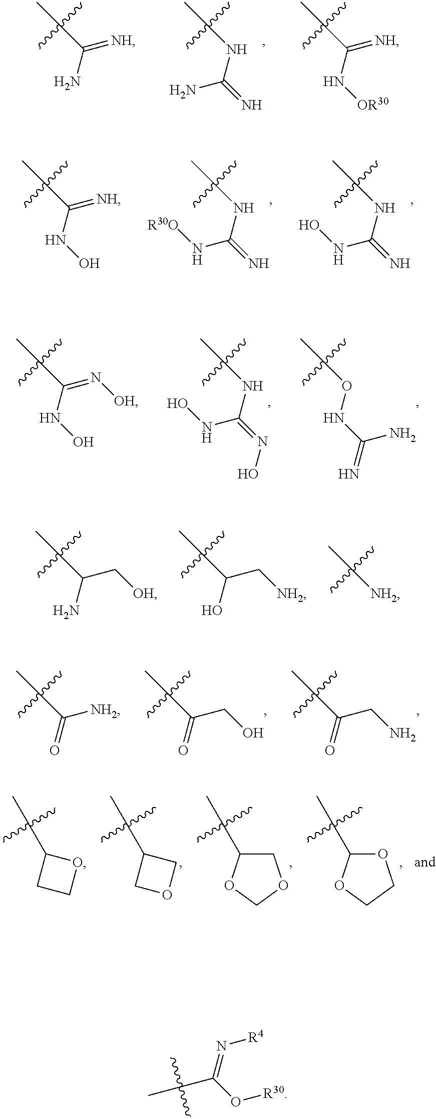

In some embodiments,

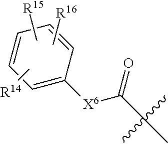

is selected from

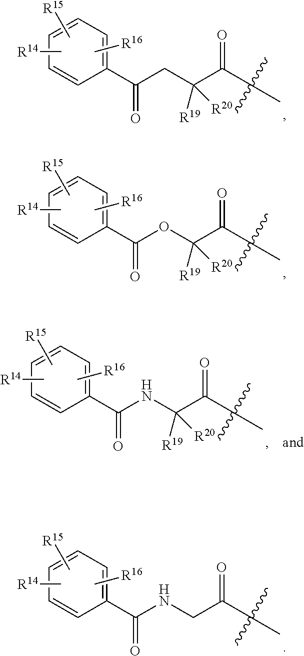

In some embodiments, R²¹ is selected from:

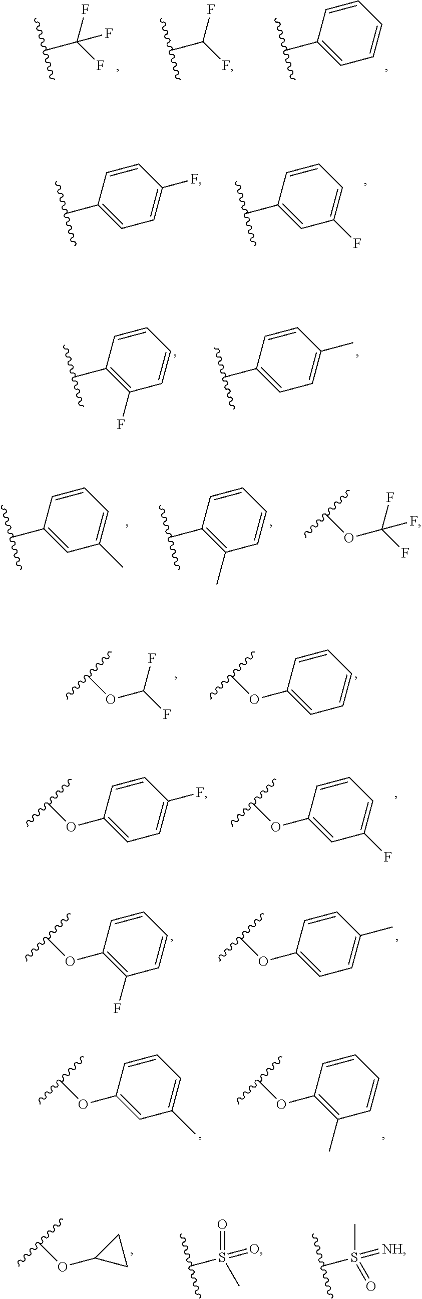

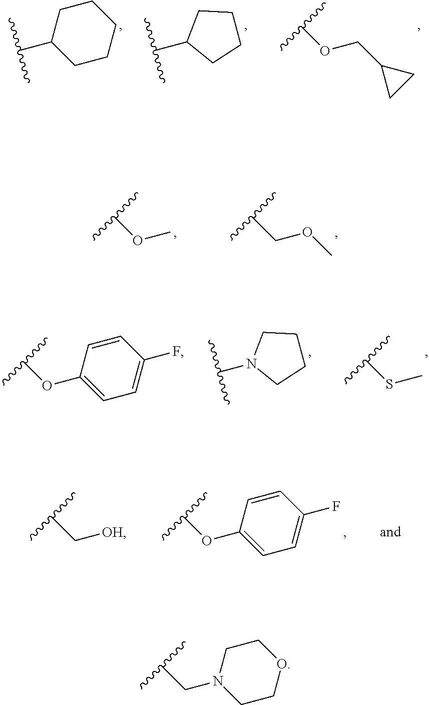

In one aspect, a compound of Formula I is selected from:

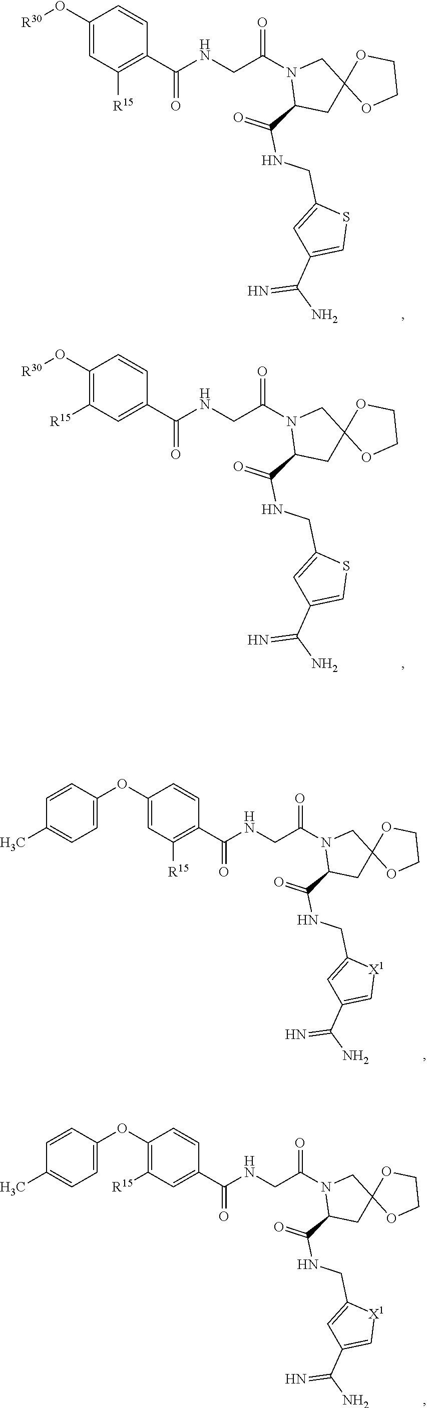

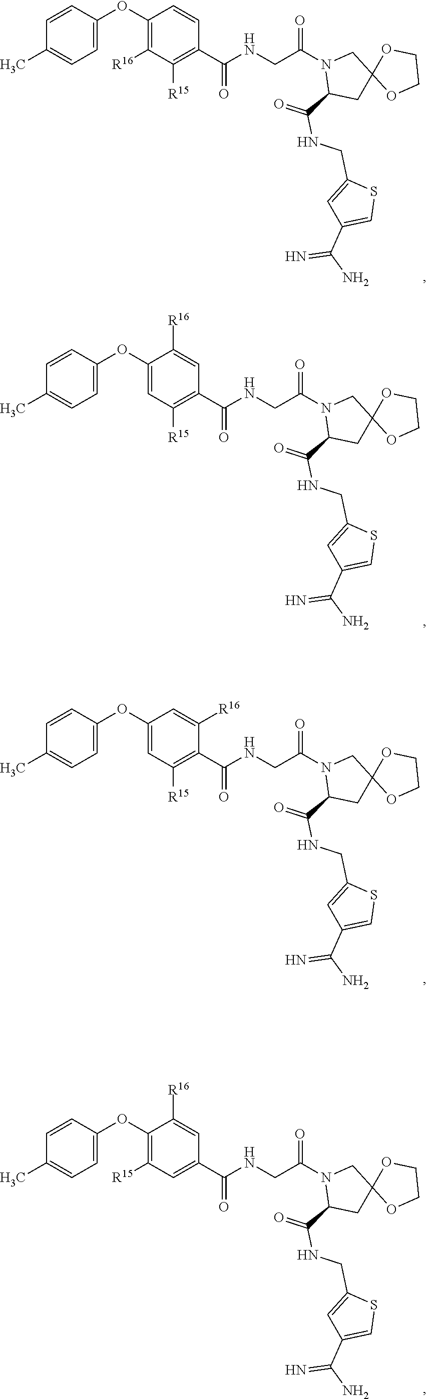

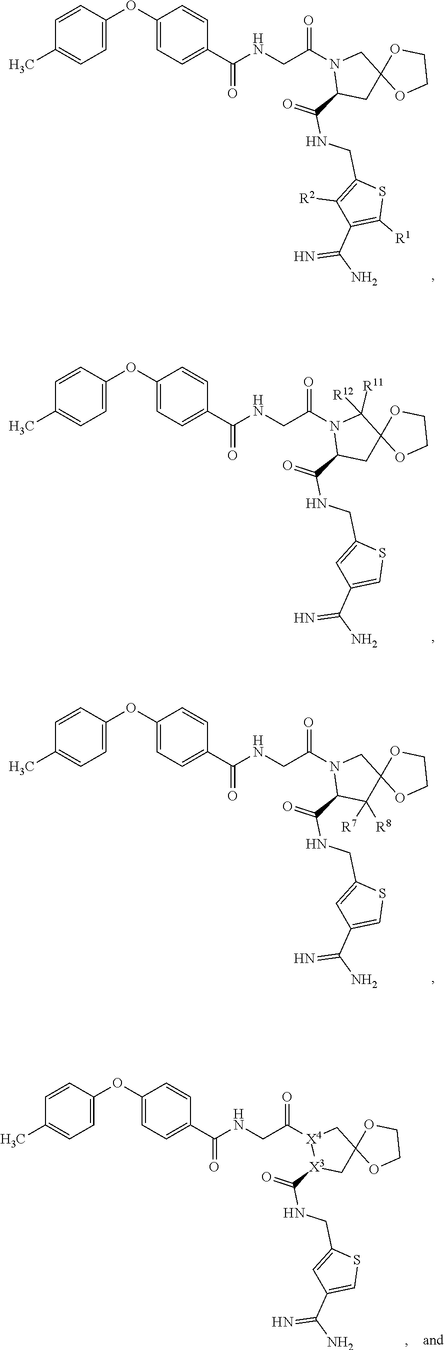

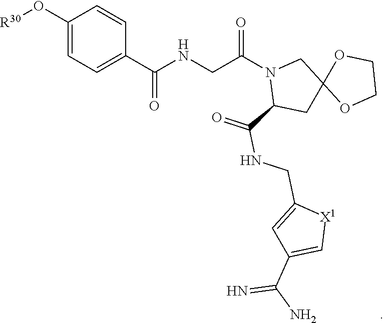

In one aspect, a compound of Formula I is selected from:

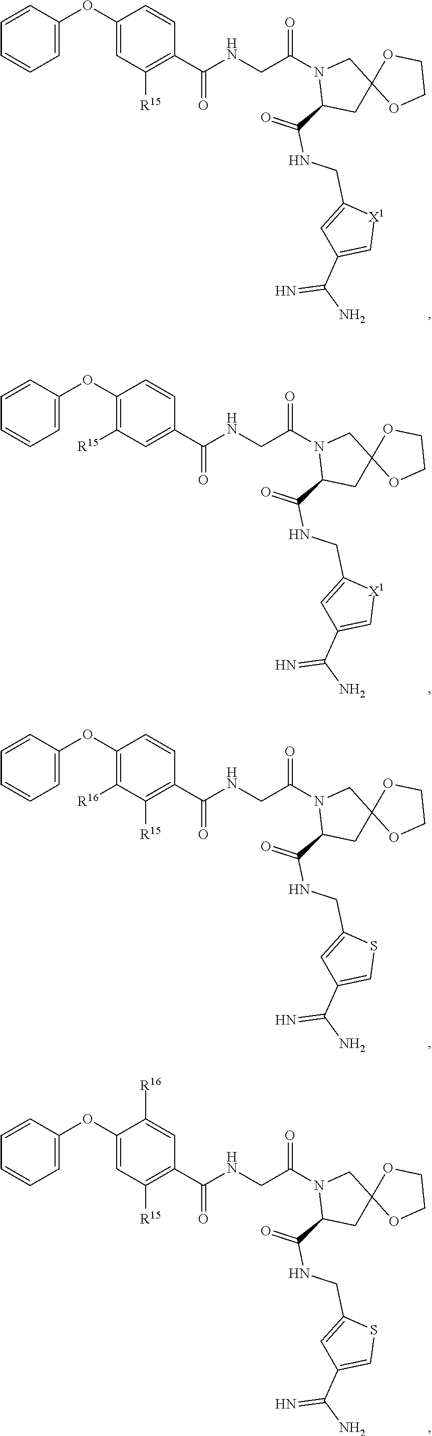

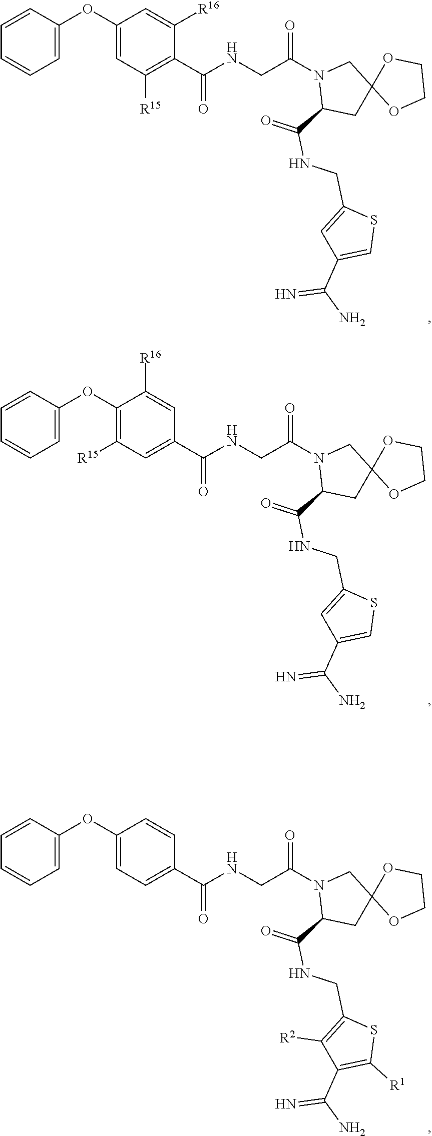

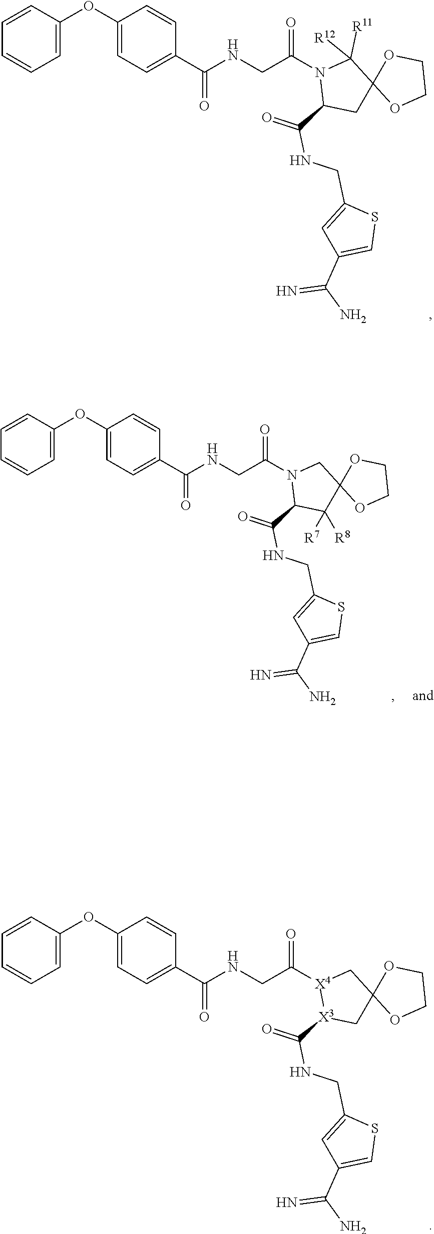

In one aspect, a compound of Formula I is selected from:

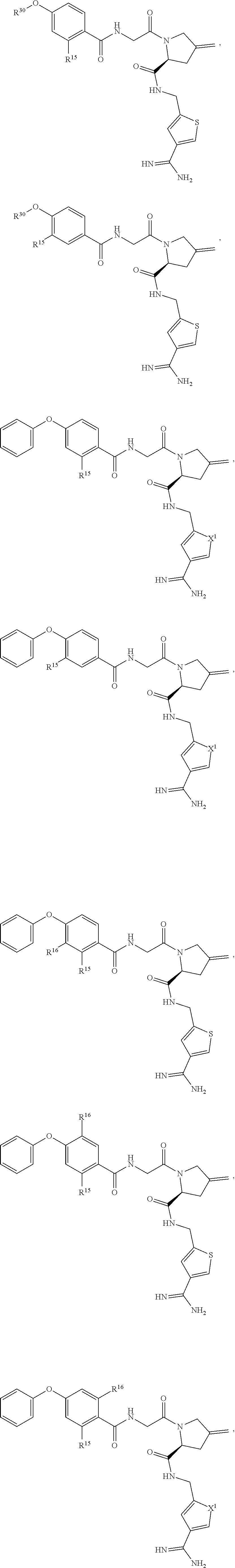

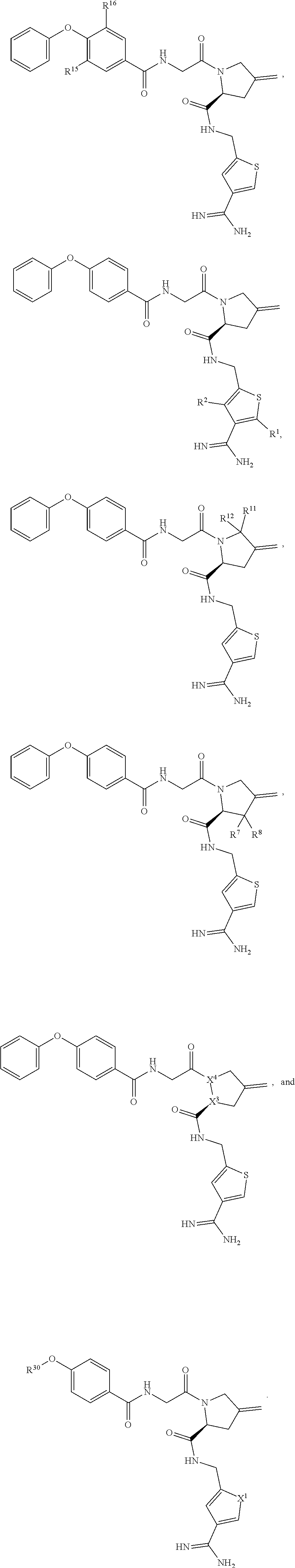

In one aspect, the compound of the present disclosure is selected from:

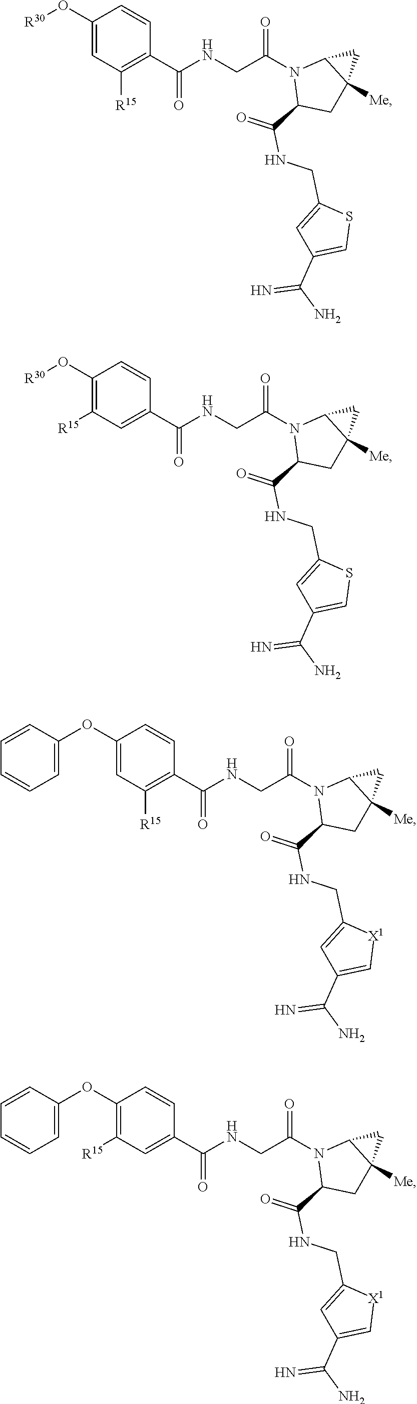

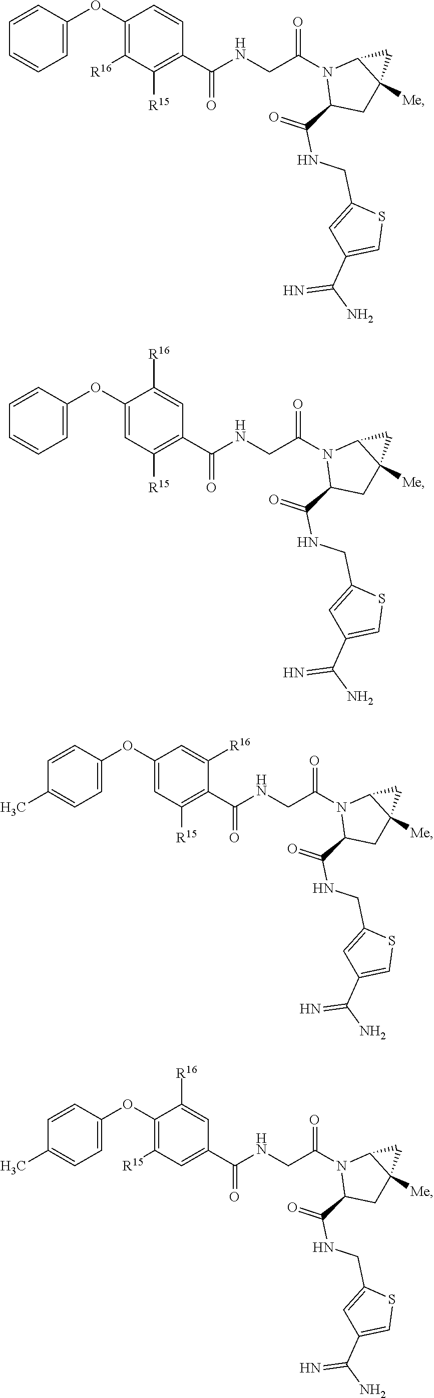

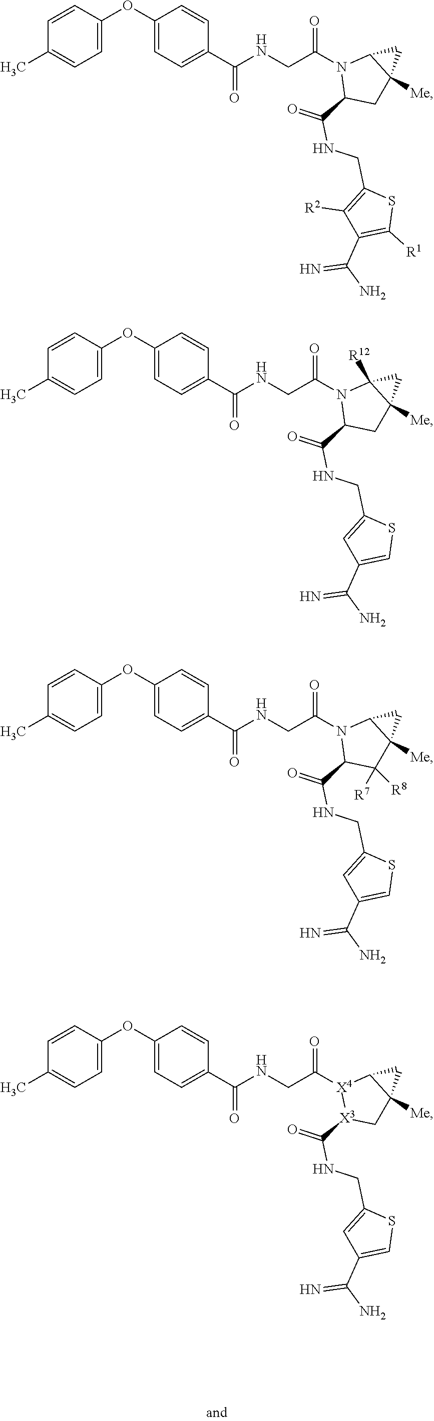

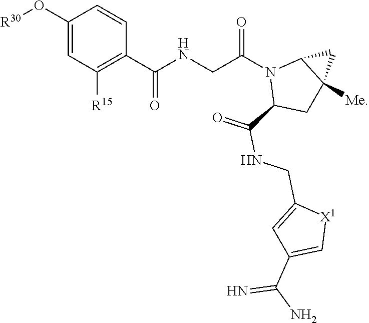

In one aspect, the compound of the present disclosure is selected from:

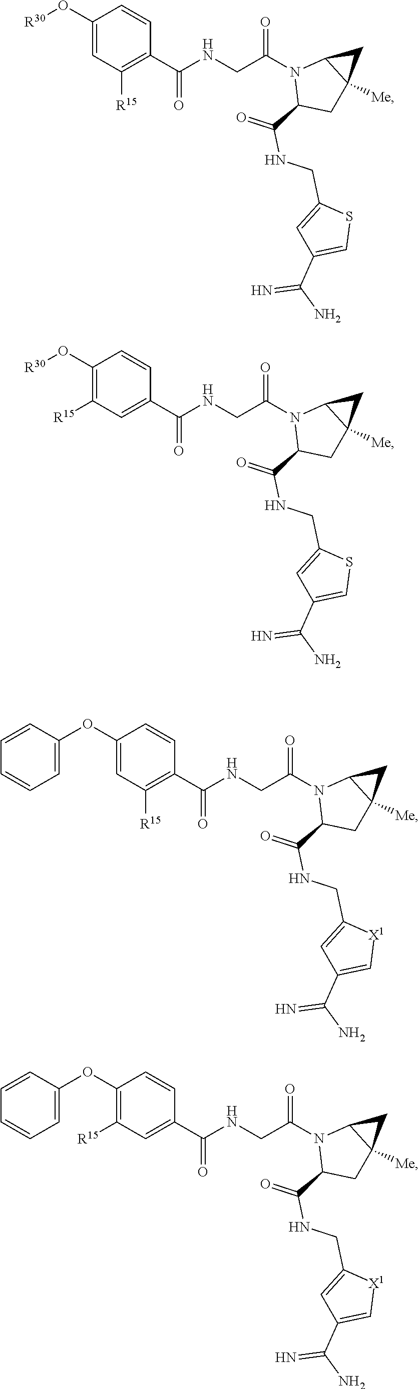

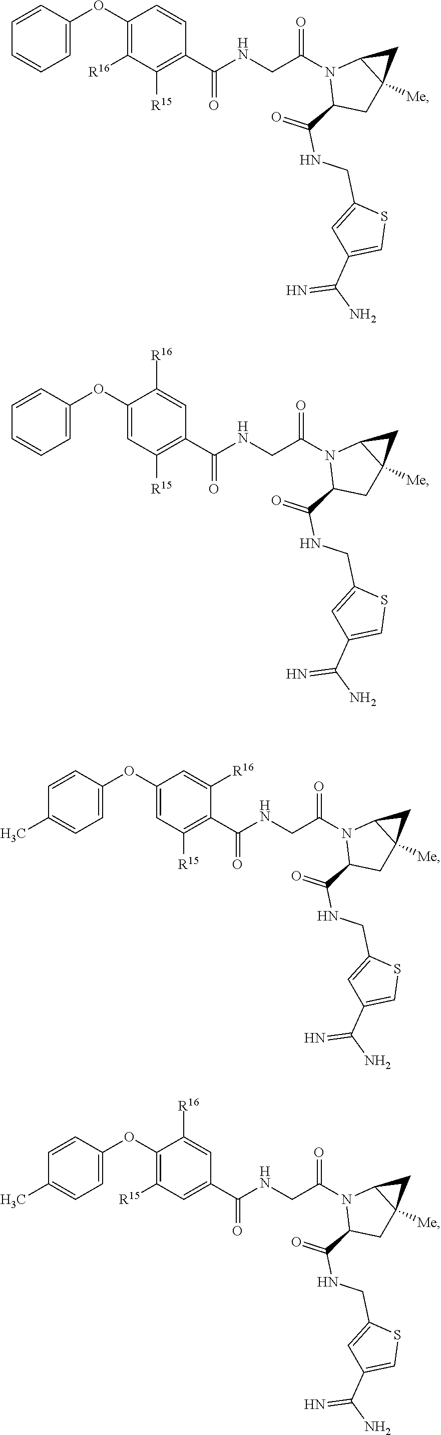

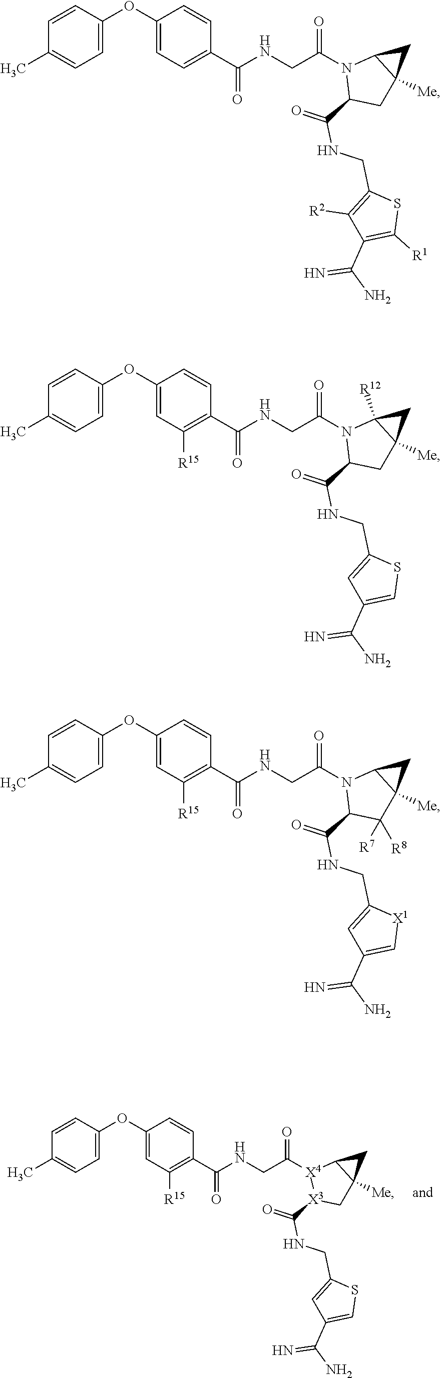

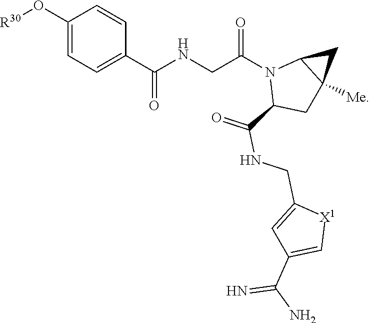

In one aspect, a compound of Formula I is selected from:

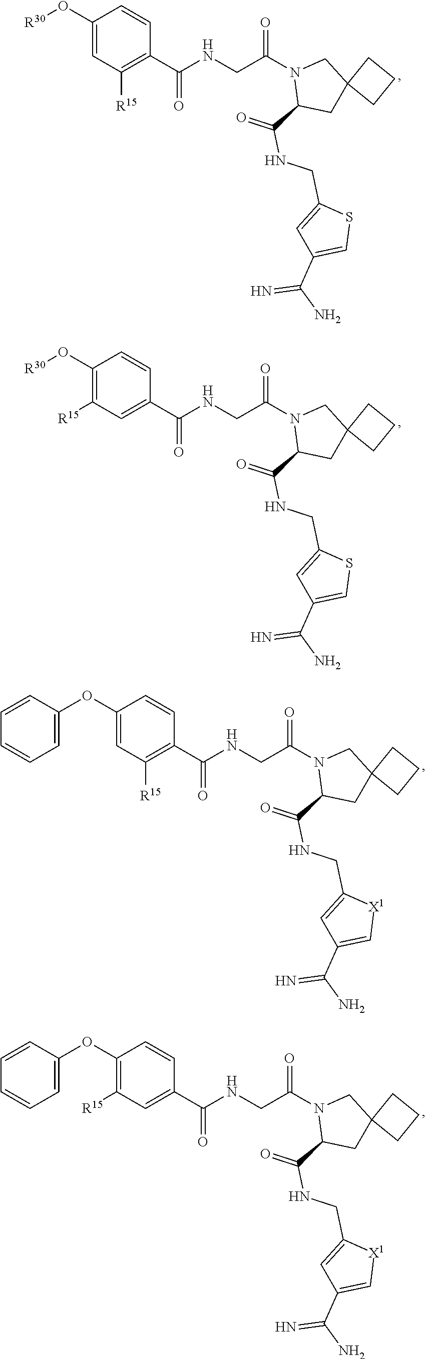

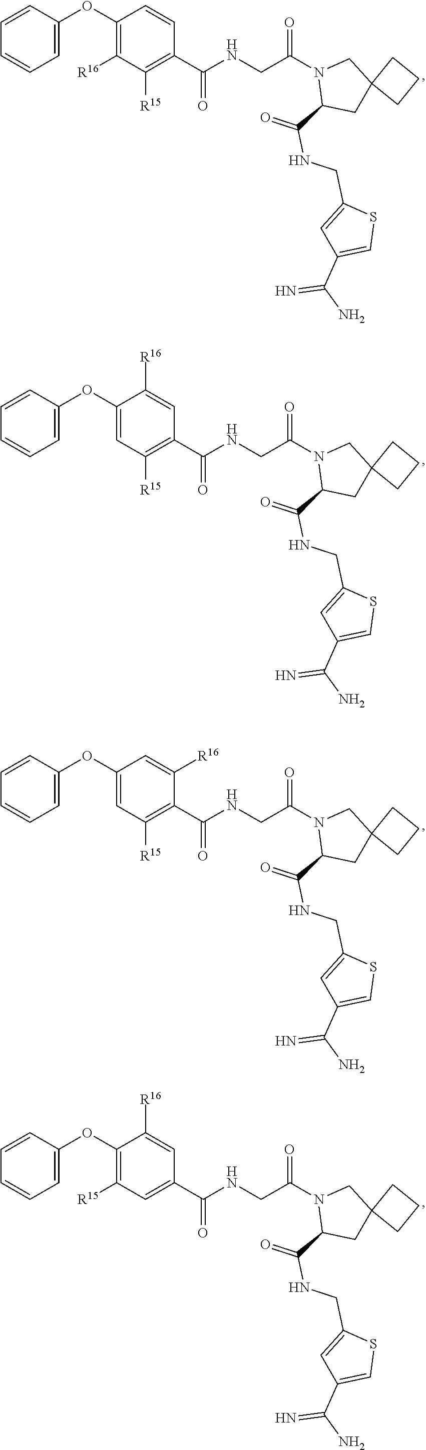

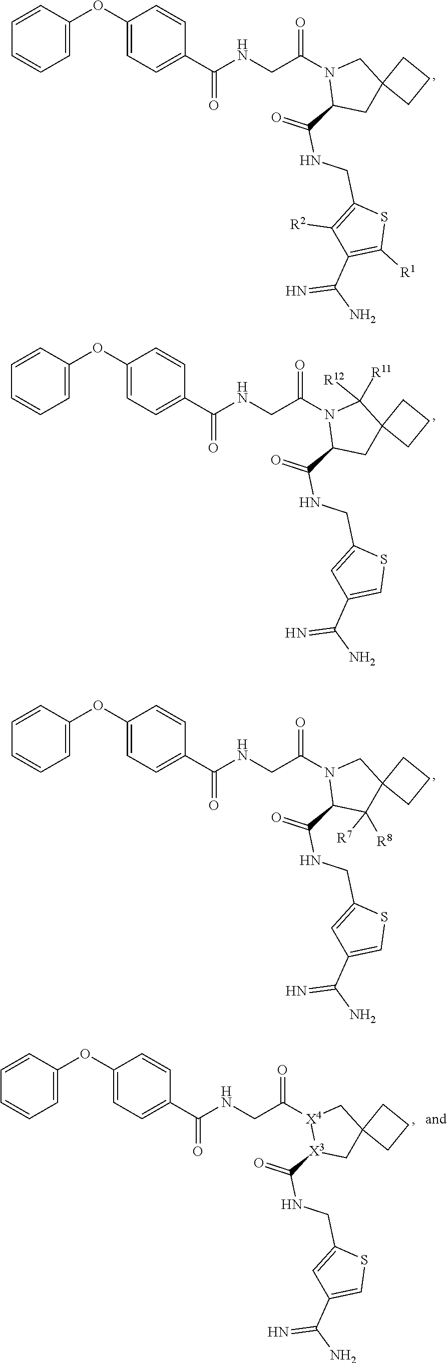

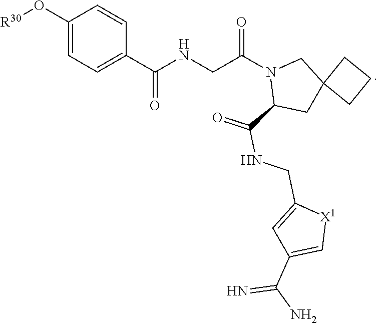

In one aspect, a compound o Formula is selected from:

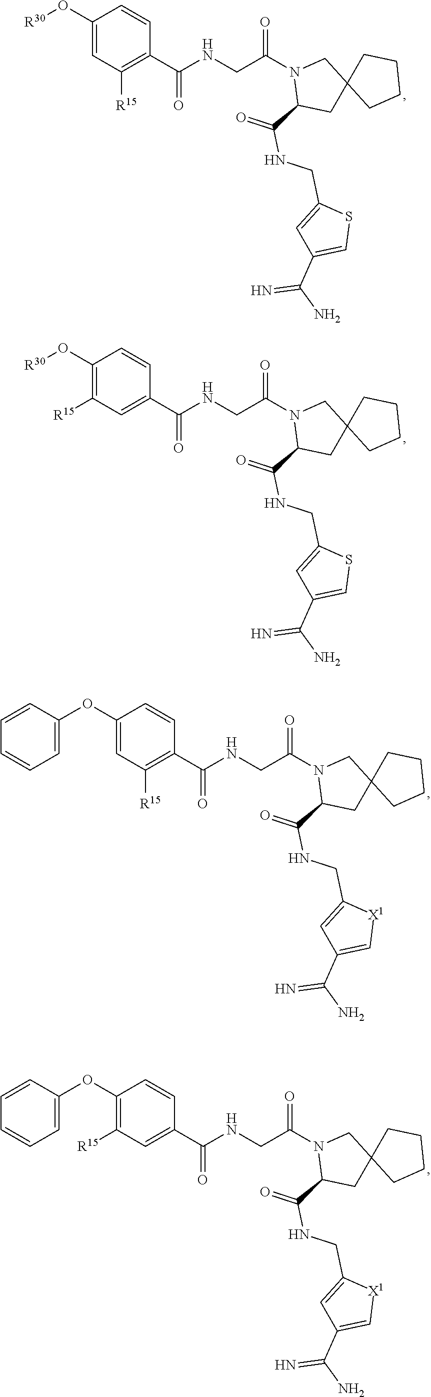

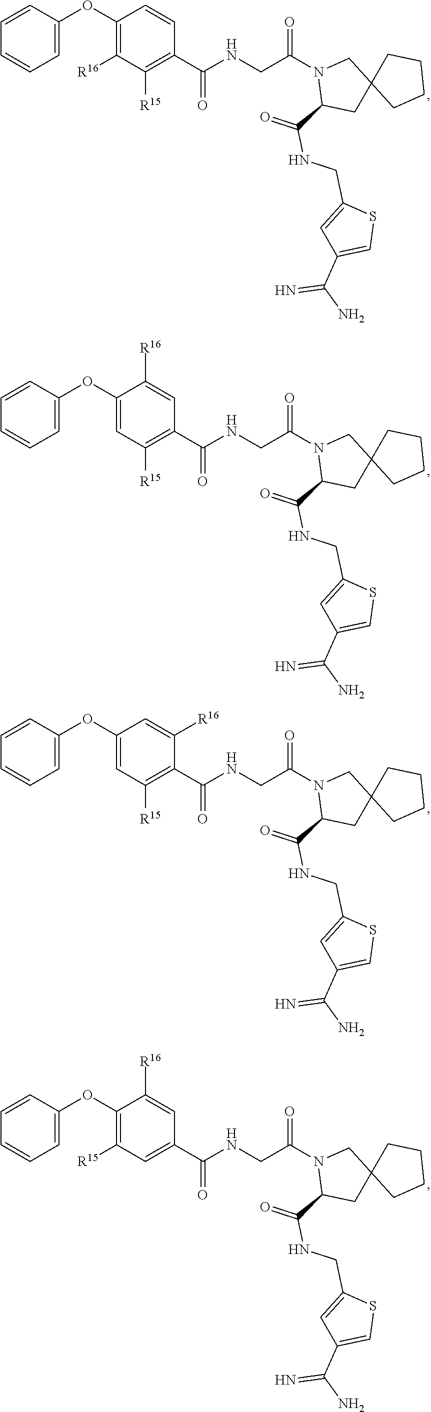

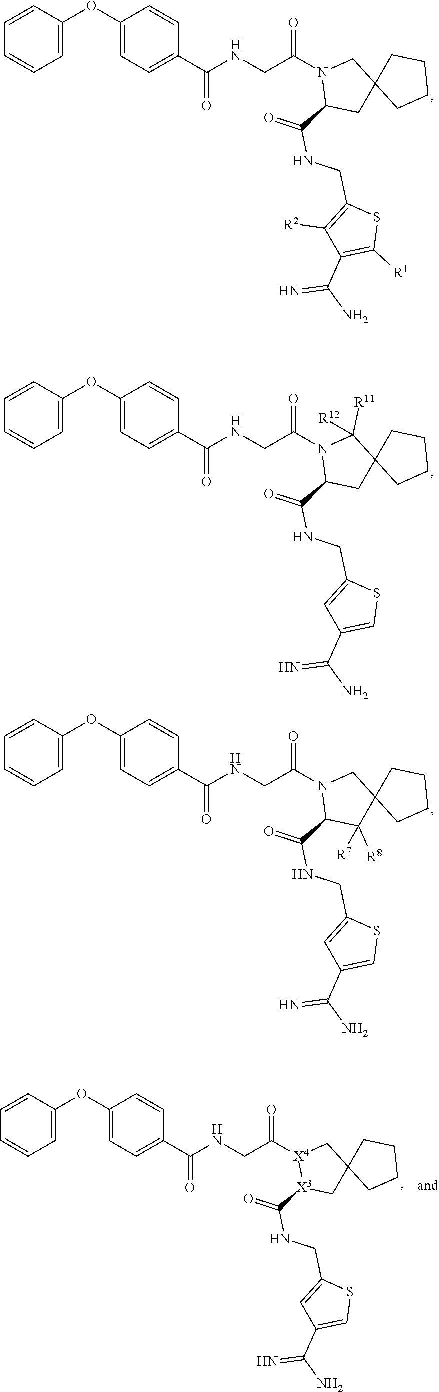

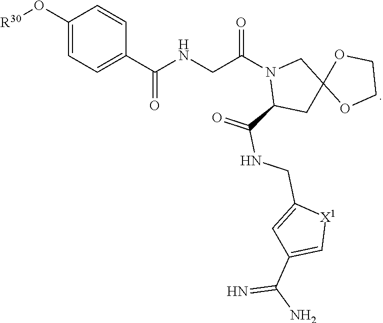

In one aspect, a compound of Formula I is selected from:

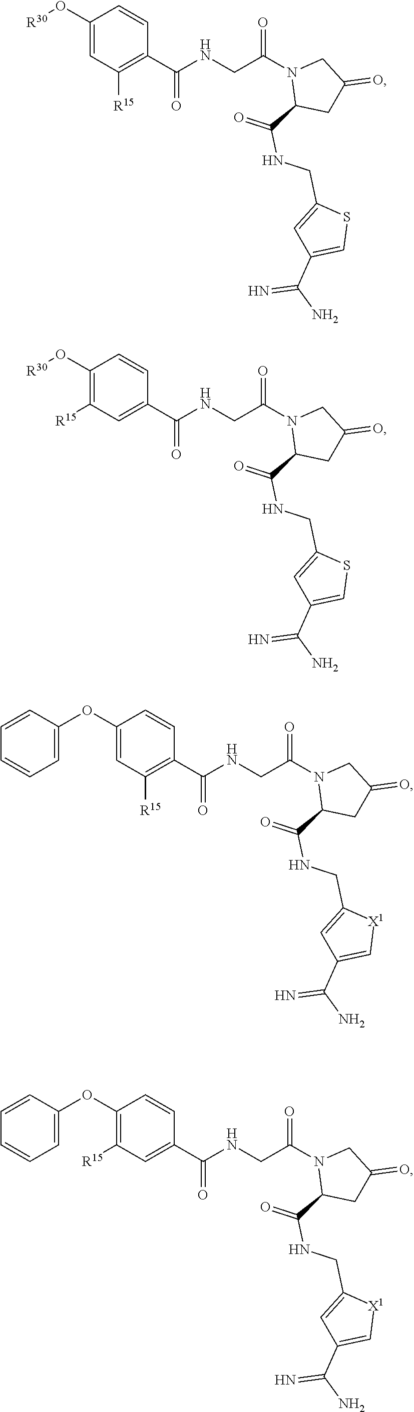

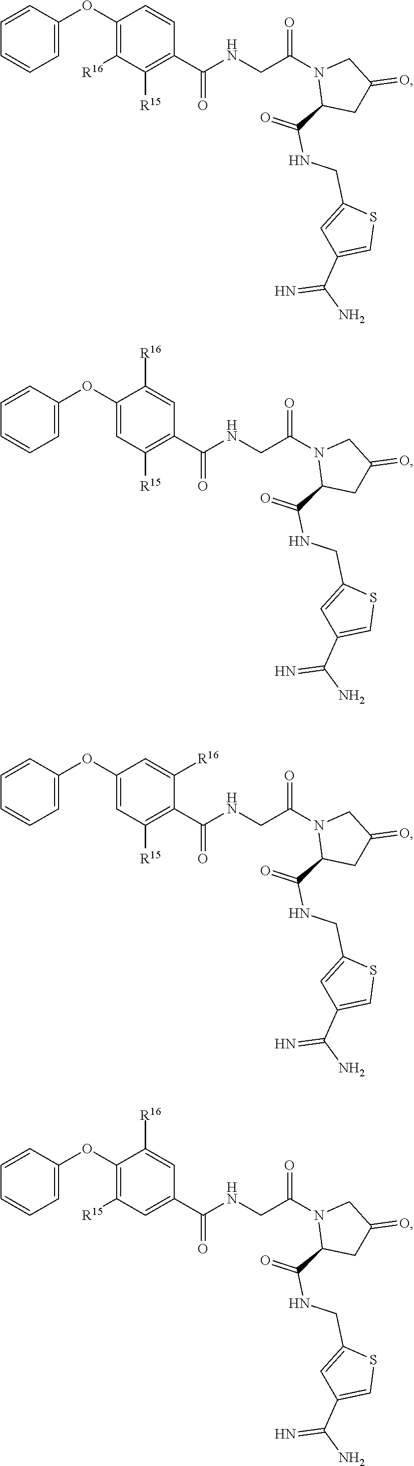

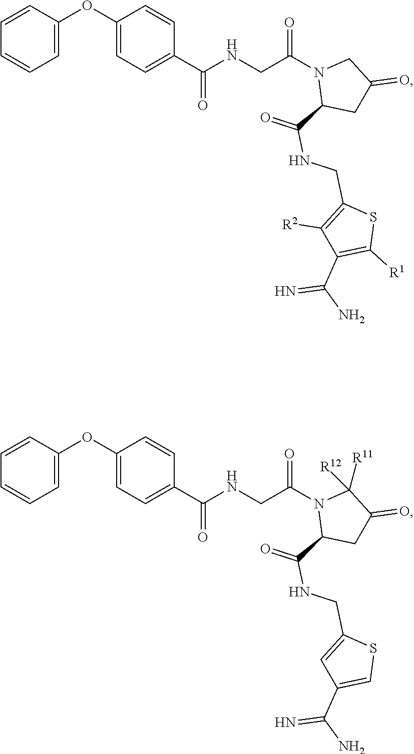

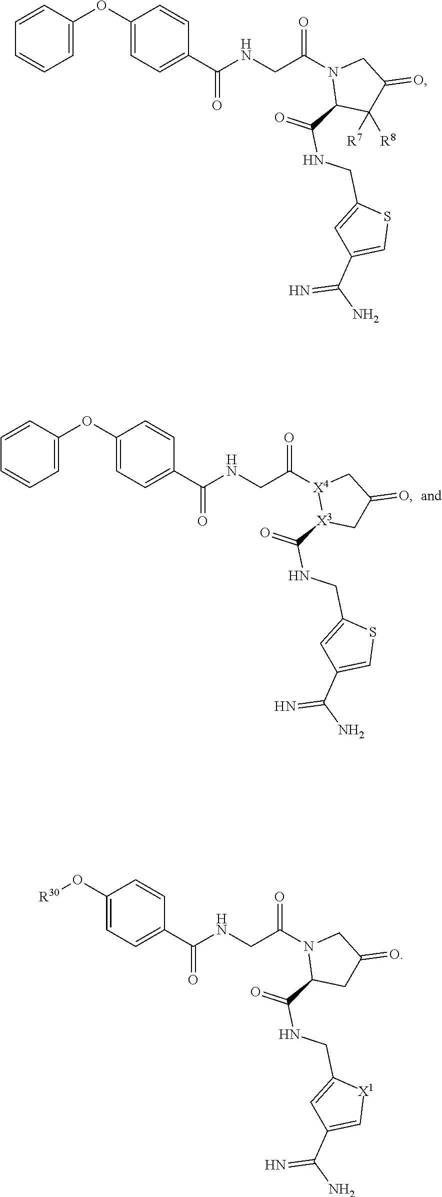

In one aspect, a compound of Formula I is selected from:

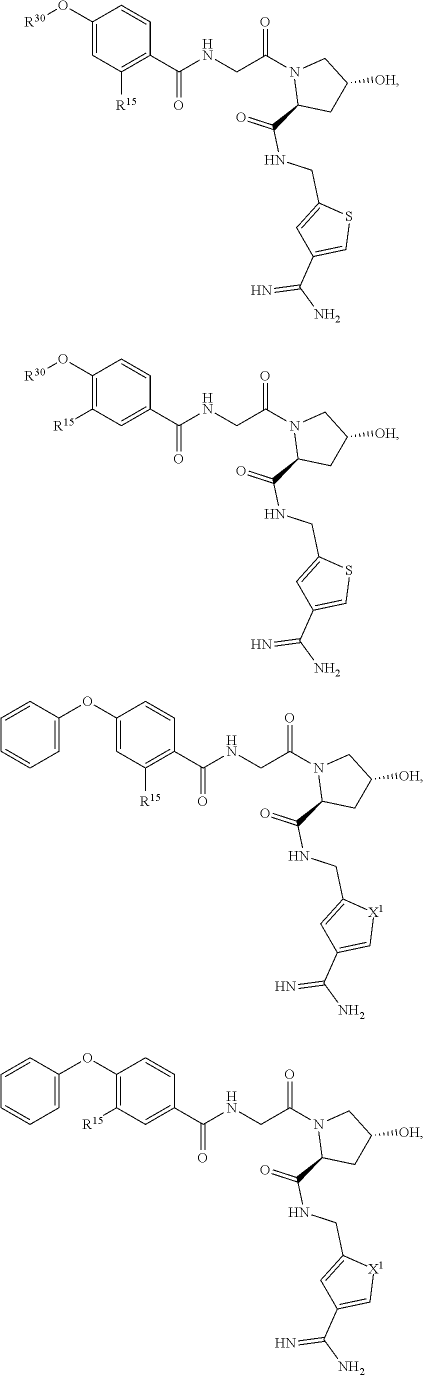

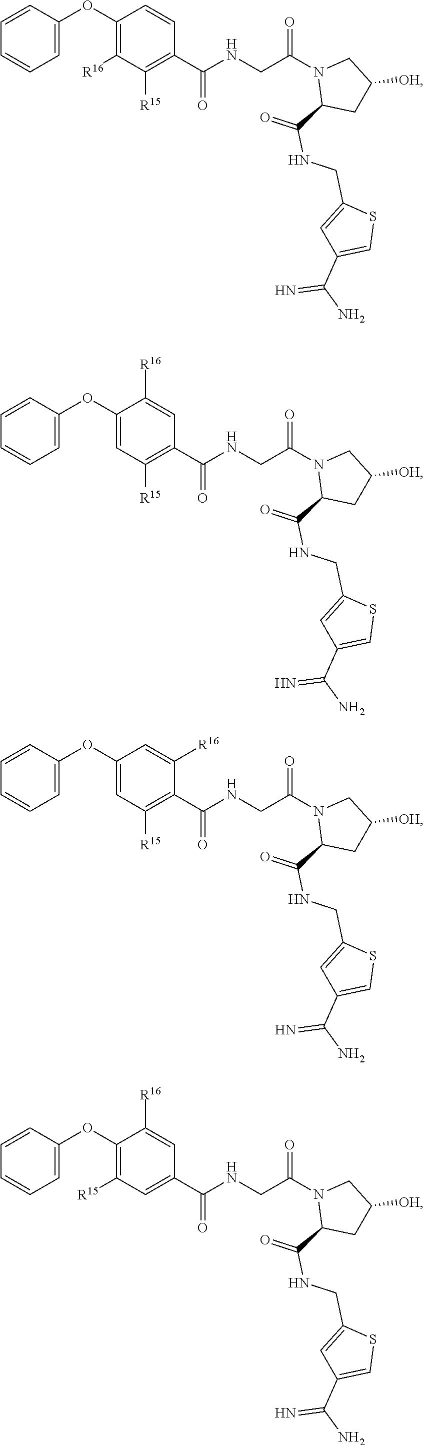

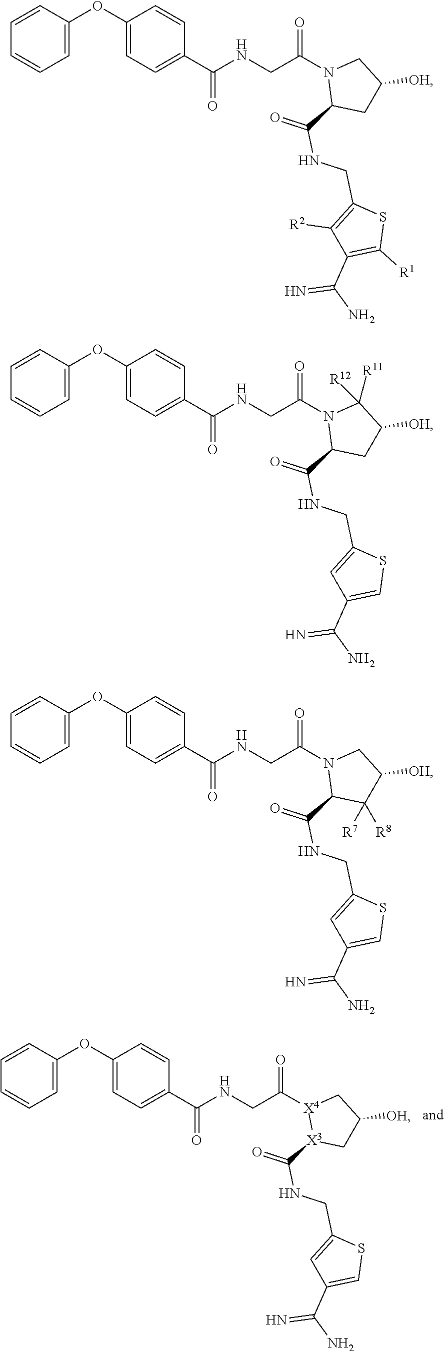

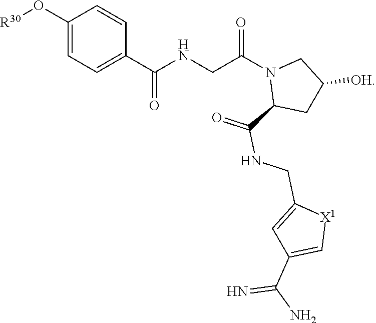

In one aspect, a compound of Formula I is selected from:

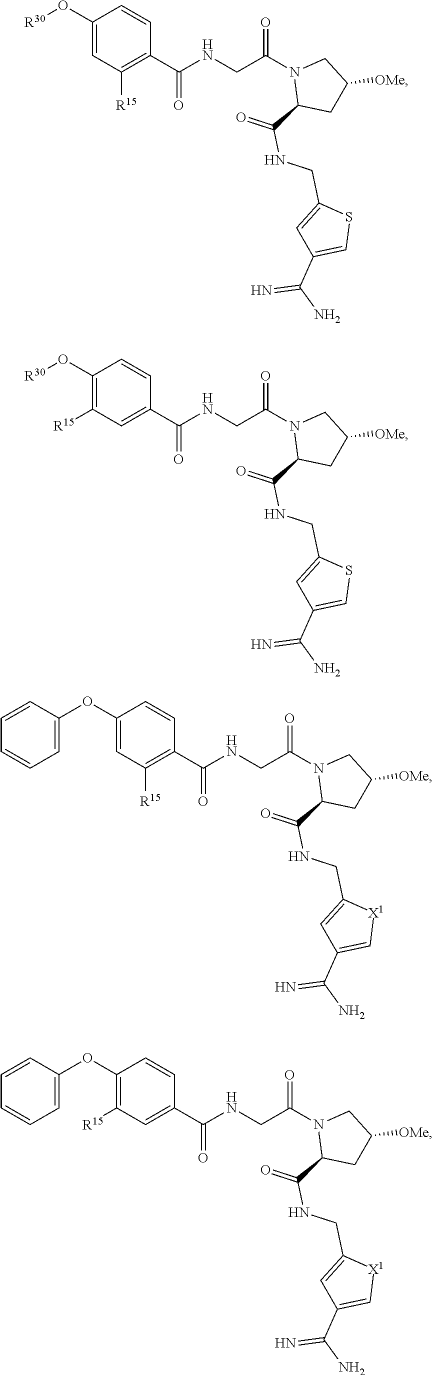

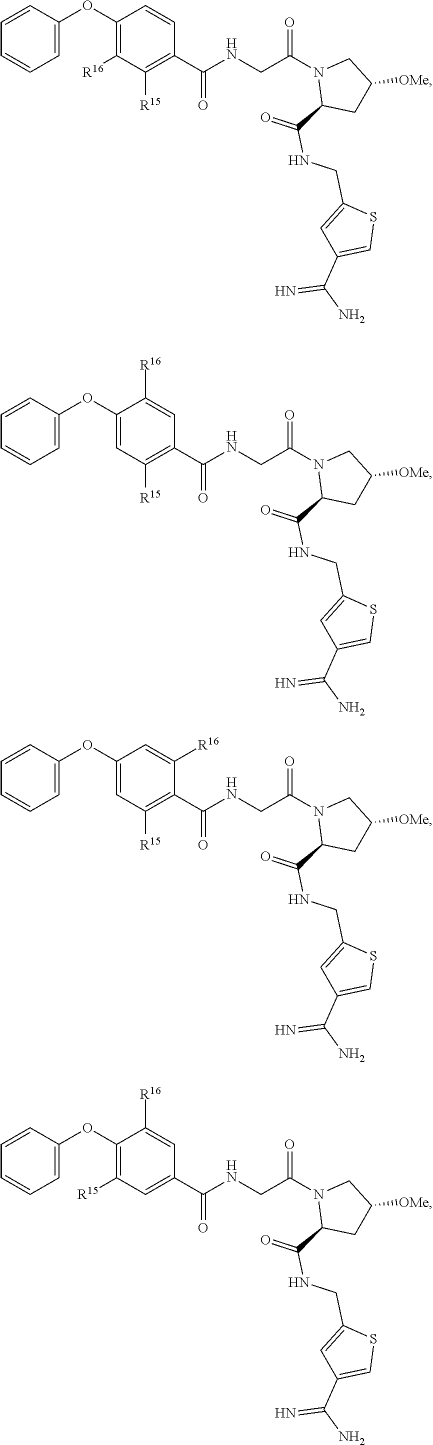

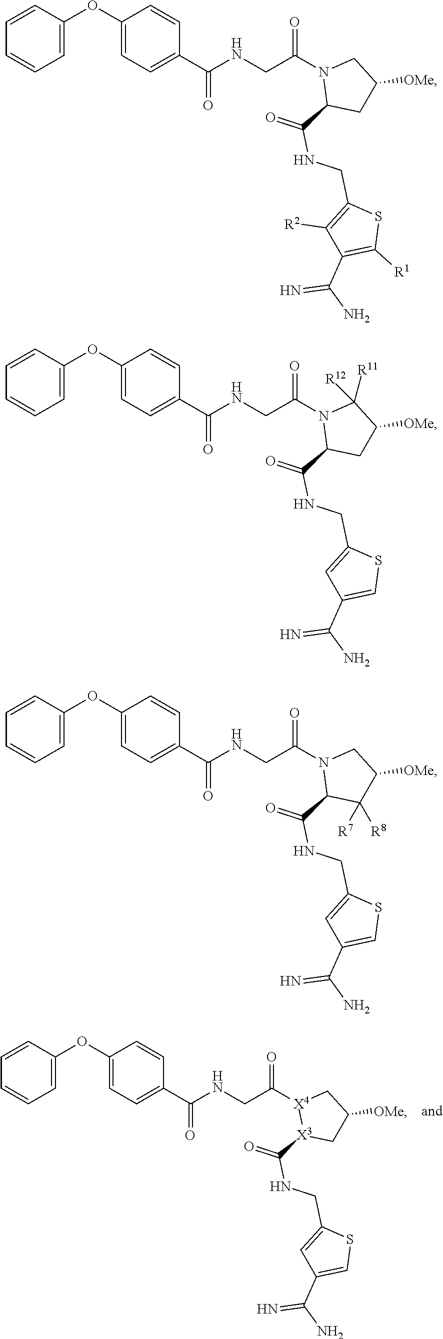

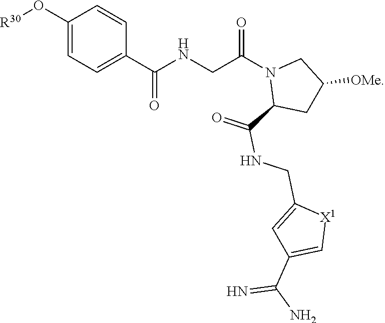

In one aspect, a compound of Formula IV is selected from:

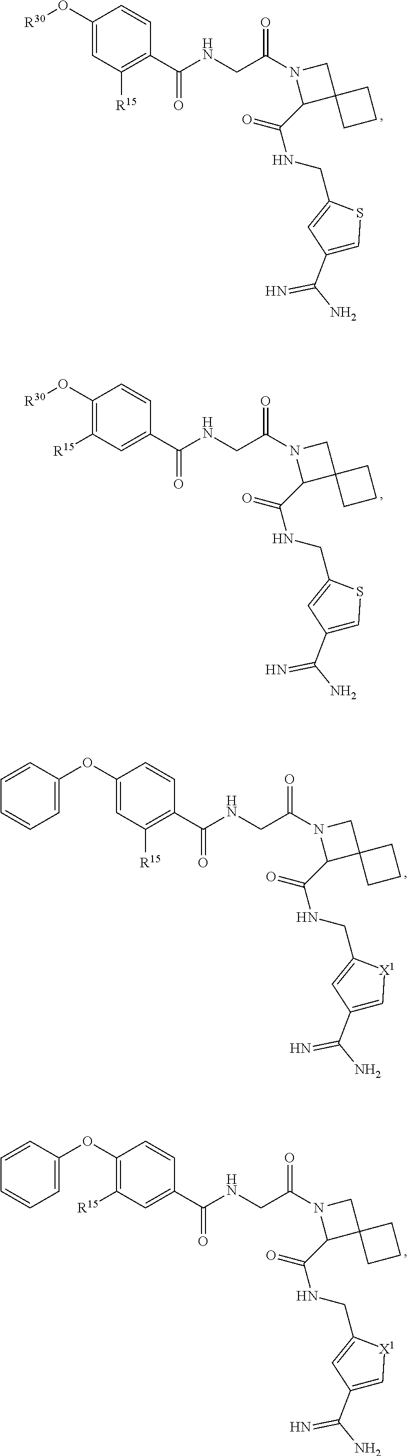

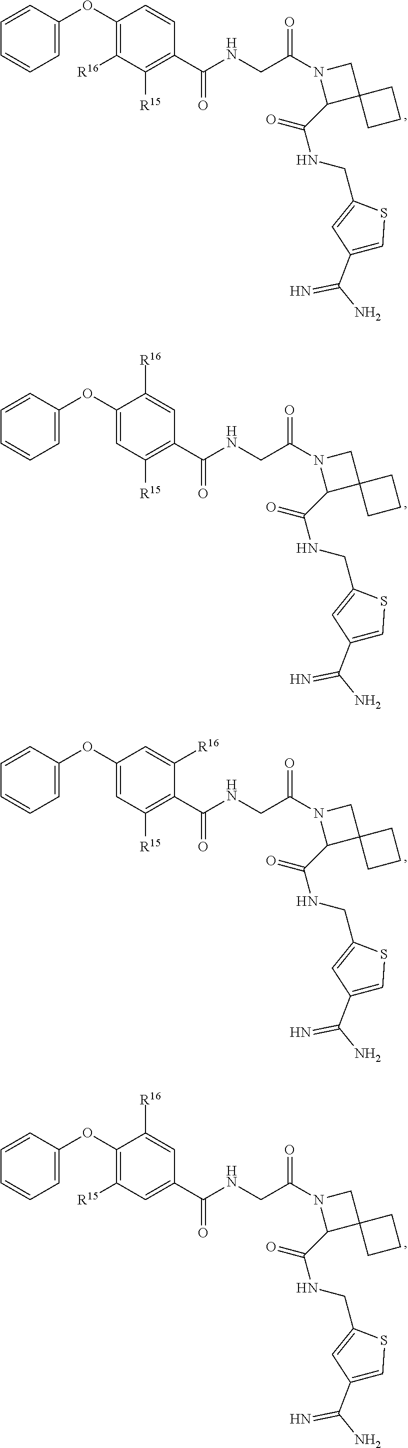

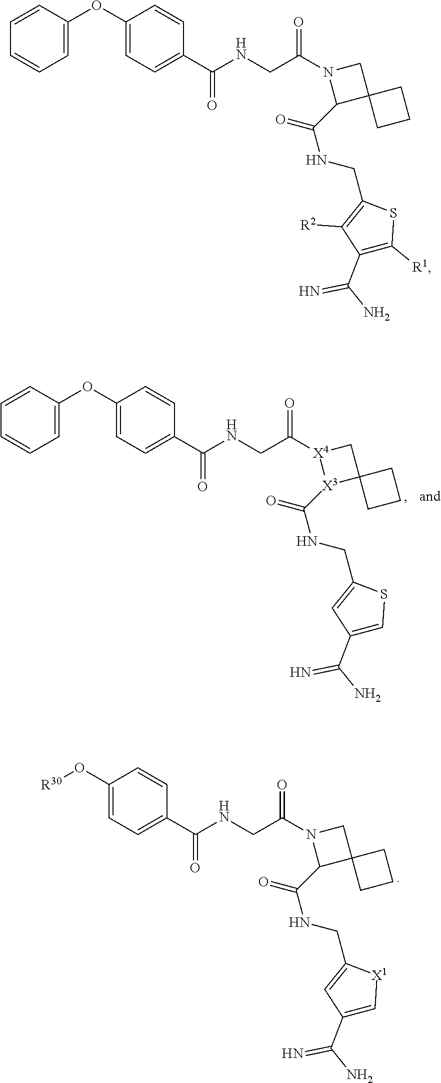

In one aspect, a compound of Formula V is selected from:

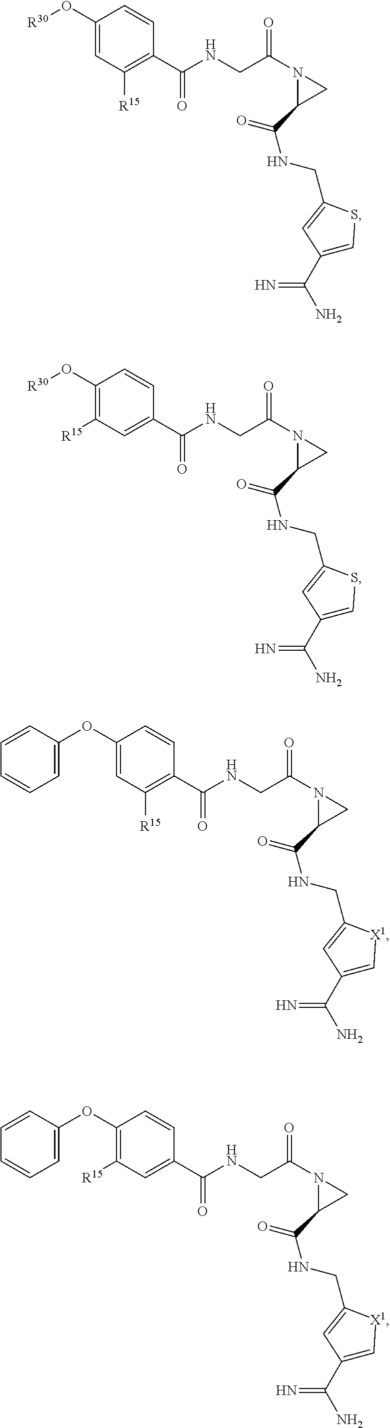

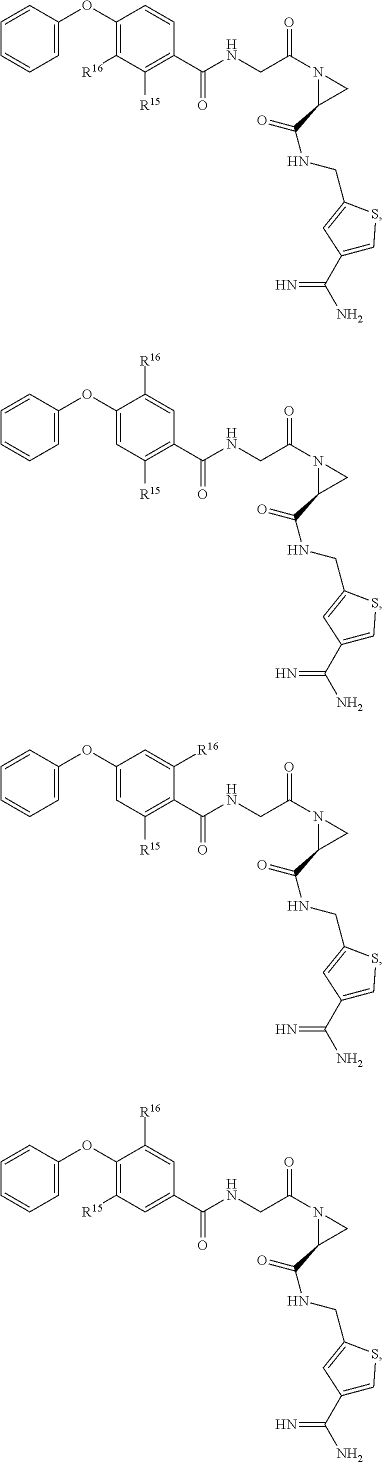

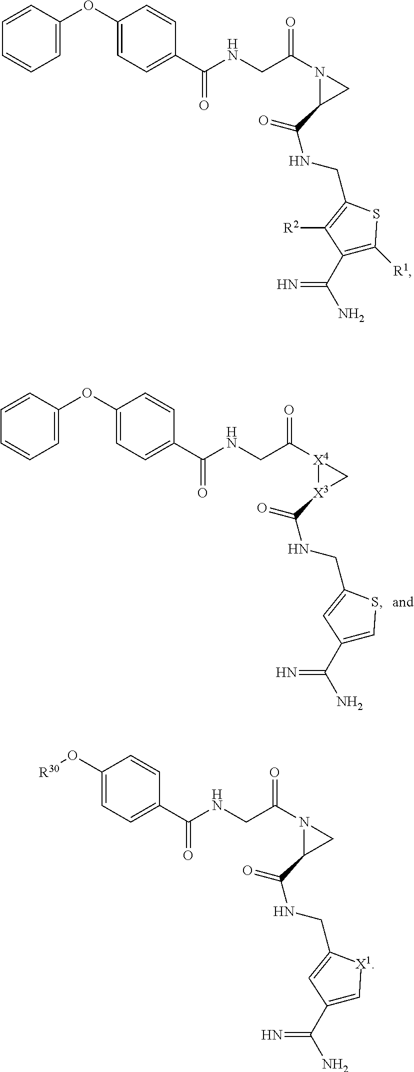

In another aspect, the compound of Formula is selected from:

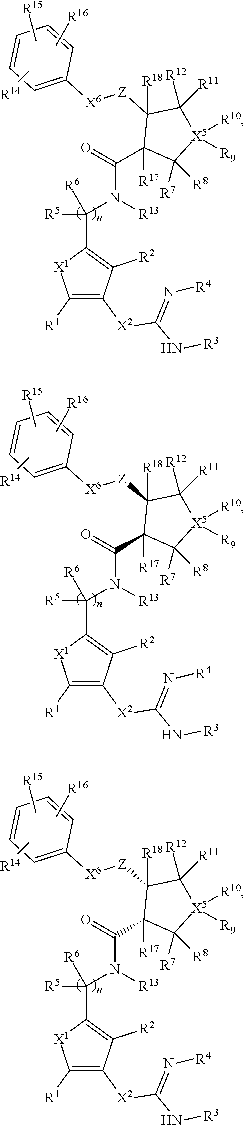

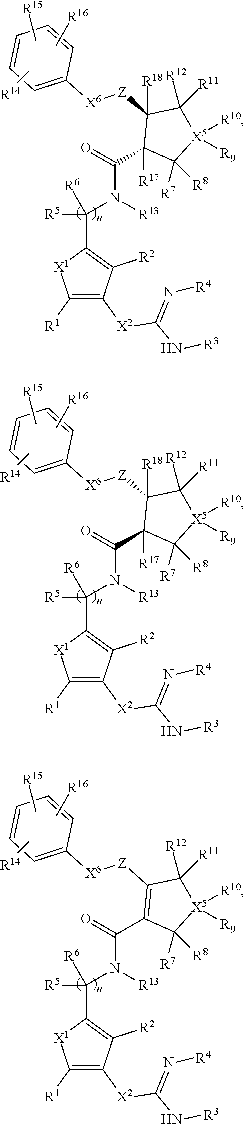

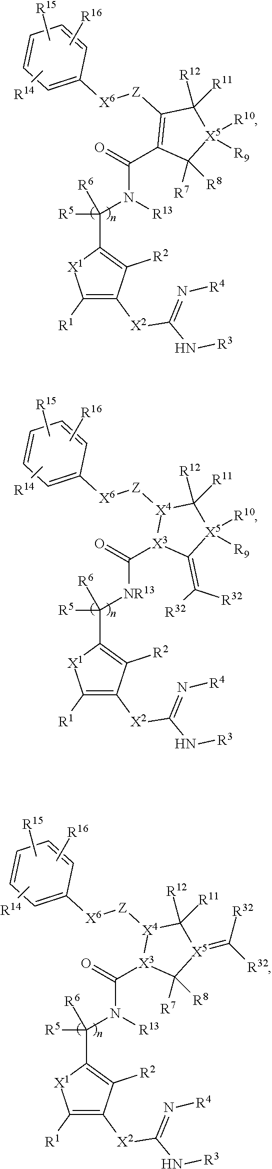

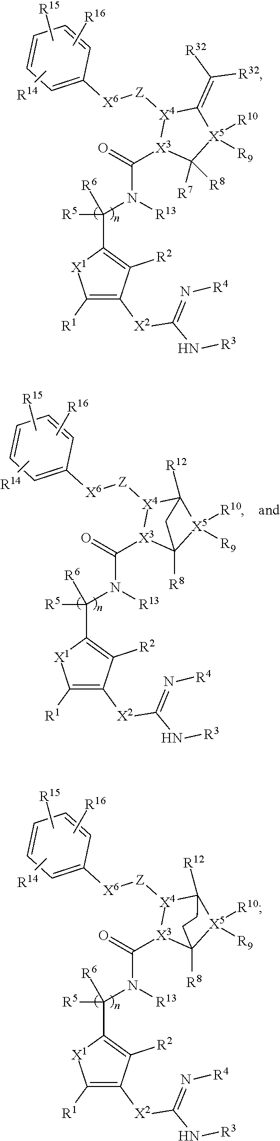

or a pharmaceutically acceptable salt, prodrug, or isolated isomer thereof, optionally in pharmaceutically acceptable carrier; wherein all variables are as defined herein.

In another aspect, the compound of Formula I is selected from:

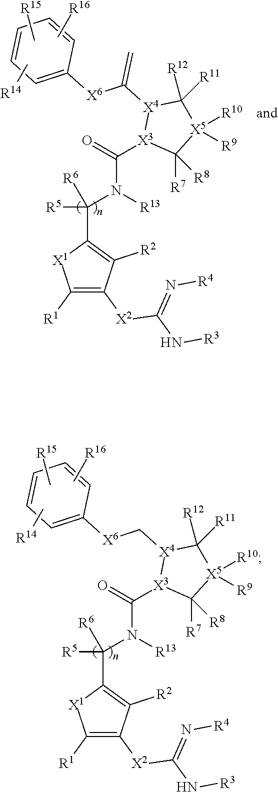

or a pharmaceutically acceptable salt, prodrug, or isolated isomer thereof, optionally in pharmaceutically acceptable carrier; wherein all variables are as defined herein.

In another aspect the compound of Formula I is selected from:

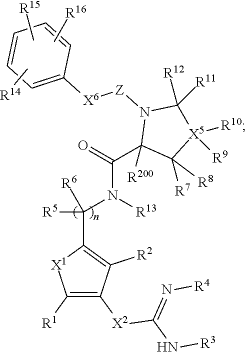

or a pharmaceutically acceptable salt, prodrug, or isolated isomer thereof, optionally in a pharmaceutically acceptable carrier;
wherein:
R²⁰⁰ is selected from halogen, C₁-C₆alkyl, C₁-C₆haloalkyl, —OR³⁰, and —N(R³⁰); and all other variables are as defined herein.

In another aspect, the compound of Formula I is selected from:

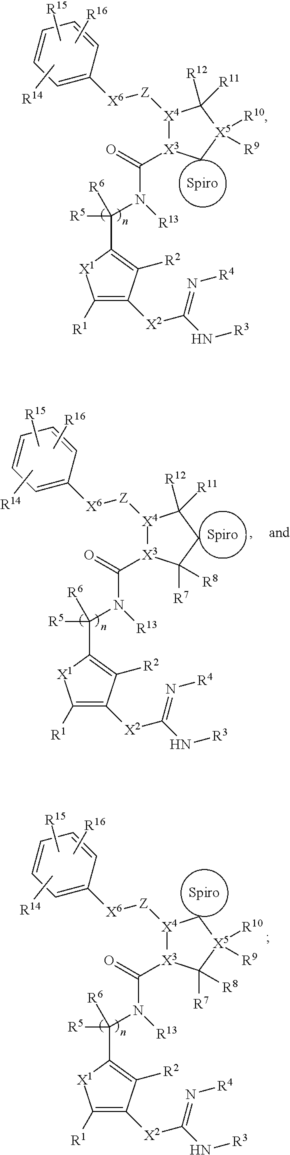

or a pharmaceutically acceptable salt, prodrug, or isolated isomer thereof, optionally in pharmaceutically acceptable carrier;
wherein:

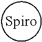

is selected from a 3- to 6-membered carbocyclic ring and a 4- to 6-membered heterocyclic ring containing 1 or 2 heteroatoms independently chosen from N, O, and S; for example

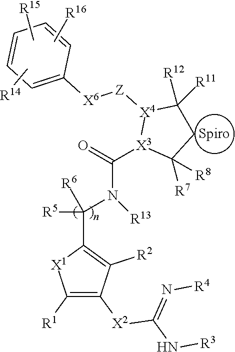

can be selected from

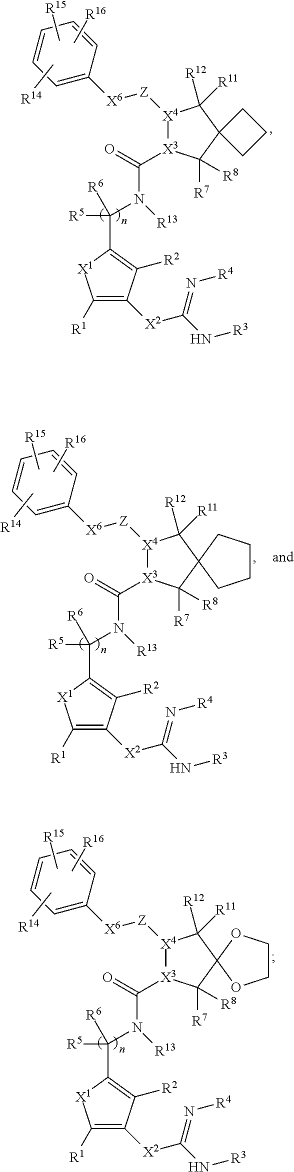

in an alternative embodiment

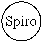

is optionally substituted with 1, 2, 3, or 4 substituents independently selected from halogen, C₁-C₆alkyl, C₁-C₆haloalkyl, —OR³⁰, and —N(R³⁰)₂;
all other variables are as defined herein.

In another aspect, the compound of Formula I is selected from:

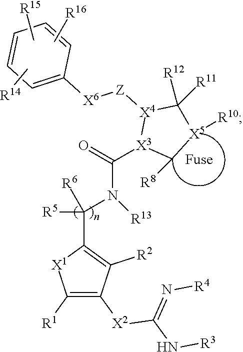

or a pharmaceutically acceptable salt, prodrug, or isolated isomer thereof, optionally in pharmaceutically acceptable carrier;
wherein:

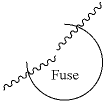

is a 3- to 8-membered carbocycle or a 4- to 8-membered heterocycle containing 1 or 2 heteroatoms independently chosen from N, O, and S; for example, in one embodiment,

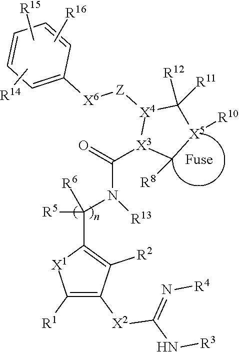

can be selected from

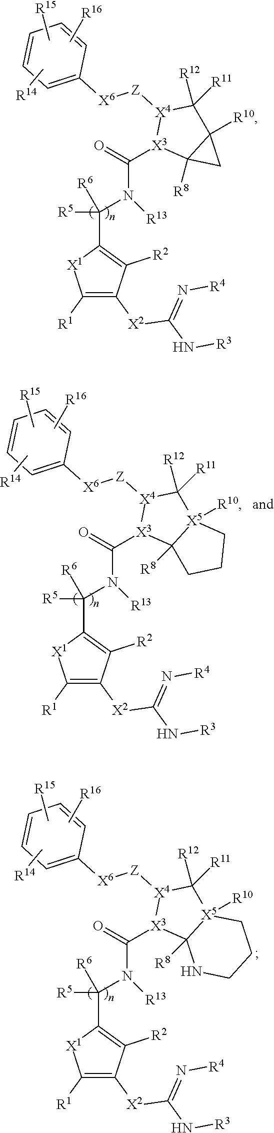

wherein in this aspect at least one of R³ and R¹⁰ is not hydrogen; and
all other variables are as defined herein.

In another aspect, the compound of Formula I is selected from:

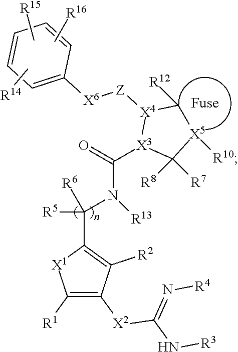

or a pharmaceutically acceptable salt, prodrug, or isolated isomer thereof, optionally in pharmaceutically acceptable carrier;
wherein:

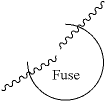

is a 3- to 8-membered carbocycle or a 4- to 8-membered heterocycle containing 1 or 2 heteroatoms independently chosen from N, O, and S; for example, in one embodiment;
wherein in this aspect at least one of R¹⁰ and R¹² is not hydrogen; and
all other variables are as defined herein.

In an alternative embodiment, the compound of the present disclosure is selected from:

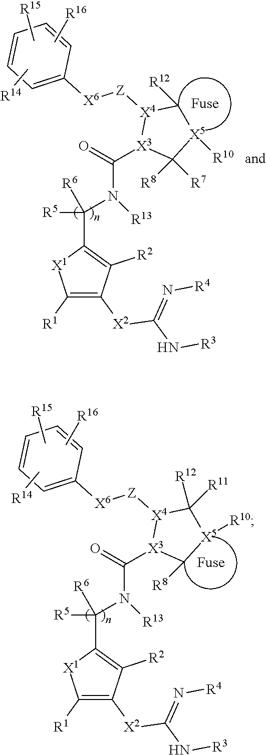

or a pharmaceutically acceptable salt, prodrug, or isolated isomer thereof, optionally in pharmaceutically acceptable carrier;
wherein:

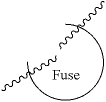

is a 3- to 8-membered carbocycle or a 4- to 8-membered heterocycle containing 1 or 2 heteroatoms independently chosen from N, O, and S, wherein

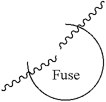

is substituted with 1, 2, 3, or 4 substituents selected from halogen, C₁-C₆alkyl, C₁-C₆haloalkyl, —OR³⁰, and —N(R³⁰)₂; and
all other variables are as defined herein.

In another aspect, the compound of Formula X is selected from:

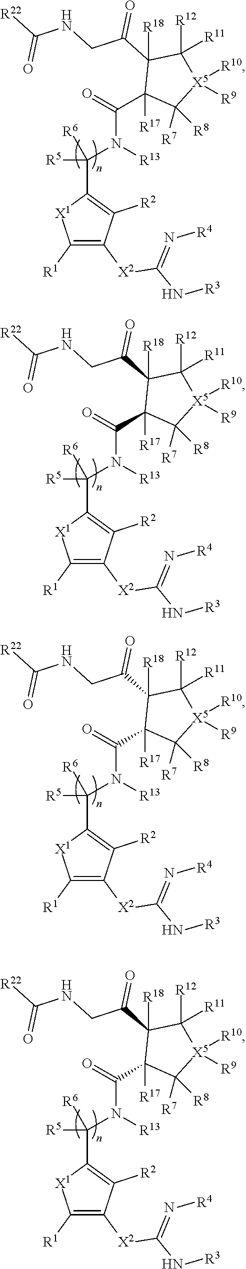

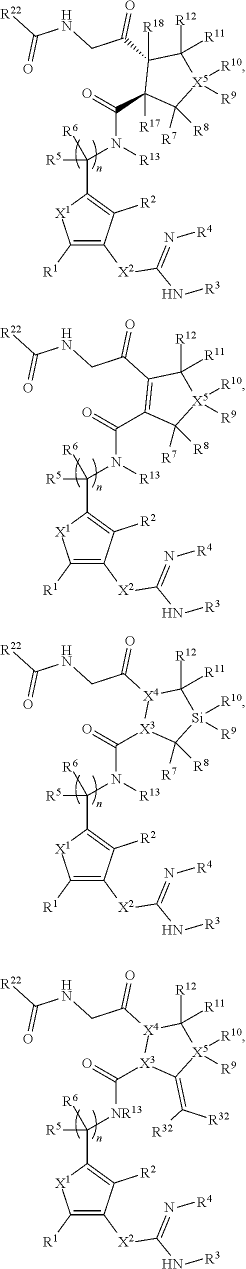

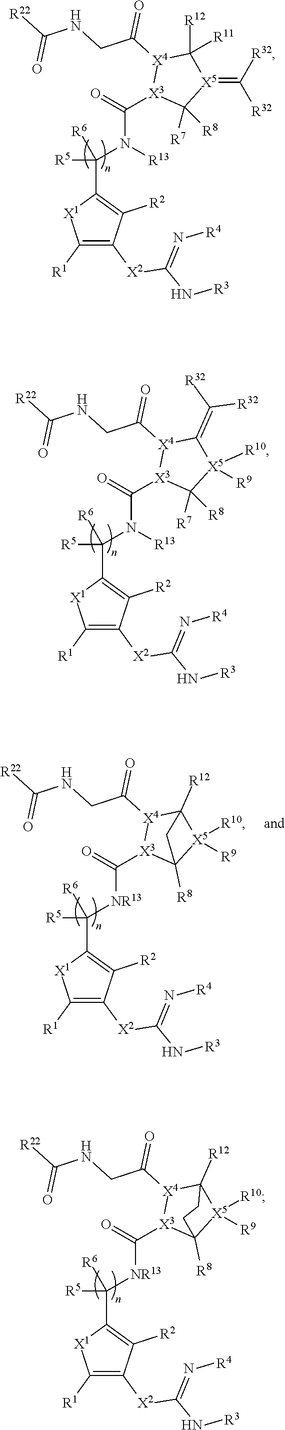

or a pharmaceutically acceptable salt, prodrug, or isolated isomer thereof, optionally in pharmaceutically acceptable carrier; wherein all variables are as defined herein.

In another aspect, the compound of Formula X is selected from:

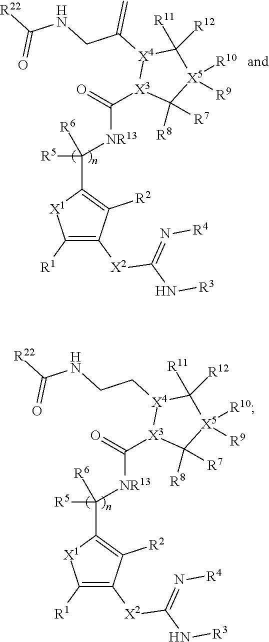

or a pharmaceutically acceptable salt, prodrug, or isolated isomer thereof, optionally in pharmaceutically acceptable carrier; wherein all variables are as defined herein.

In another aspect, the compound of Formula X is selected from:

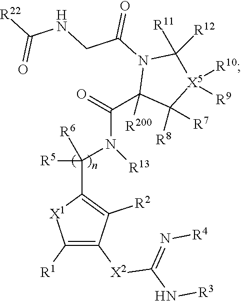

or a pharmaceutically acceptable salt, prodrug, or isolated isomer thereof, optionally in pharmaceutically acceptable carrier;
wherein:
R²⁰⁰ is selected from halogen, C₁-C₆alkyl, C₁-C₆haloalkyl, —OR³⁰, and —N(R³⁰) and all other variables are as defined herein.

In another aspect the compound of Formula X is selected from:

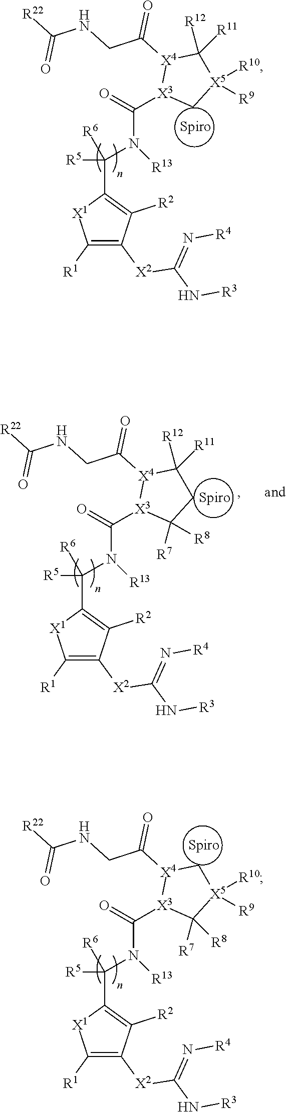

or a pharmaceutically acceptable salt, prodrug, or isolated isomer thereof, optionally in pharmaceutically acceptable carrier;
wherein:

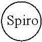

is selected from a 3- to 6-membered carbocyclic ring and a 4- to 6-membered heterocyclic ring containing 1 or 2 heteroatoms independently chosen from N, O, and S;
in an alternative embodiment

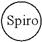

is optionally substituted with 1, 2, 3, or 4 substituents independently selected from halogen, C₁-C₆alkyl, C₁-C₆haloalkyl, —OR³⁰, and —N(R³⁰)₂;
all other variables are as defined herein.

In another aspect, the compound of Formula X is selected from:

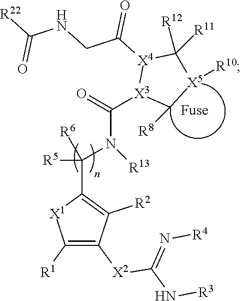

or a pharmaceutically acceptable salt, prodrug, or isolated isomer thereof, optionally in pharmaceutically acceptable carrier;
wherein:

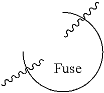

is a 3- to 8-membered carbocycle or a 4- to 8-membered heterocycle containing 1 or 2 heteroatoms independently chosen from N, O, and S;
wherein in this aspect at least one of R⁸ and R¹⁰ is not hydrogen; and
all other variables are as defined herein.

In another aspect, the compound of Formula X is selected from:

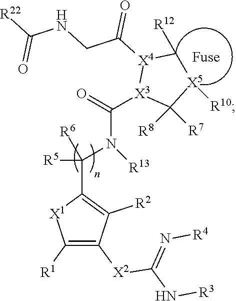

or a pharmaceutically acceptable salt, prodrug, or isolated isomer thereof, optionally in pharmaceutically acceptable carrier;
wherein:

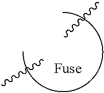

is a 3- to 8-membered carbocycle or a 4- to 8-membered heterocycle containing 1 or 2 heteroatoms independently chosen from N, O, and S; for example, in one embodiment
wherein in this aspect at least one of R¹⁰ and R¹² is not hydrogen; and
all other variables are as defined herein.

In an alternative embodiment, the compound of the present disclosure is selected from:

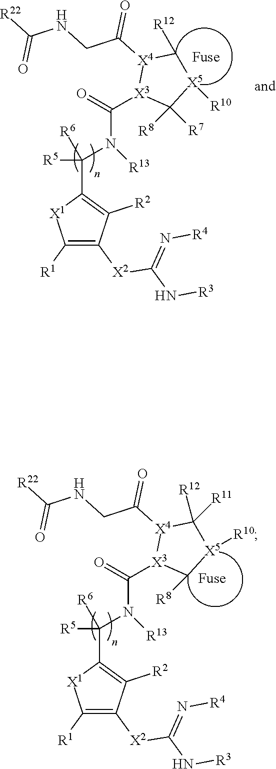

or a pharmaceutically acceptable salt, prodrug, or isolated isomer thereof, optionally in pharmaceutically acceptable carrier;
wherein:

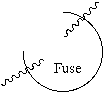

is a 3- to 8-membered carbocycle or a 4- to 8-membered heterocycle containing 1 or 2 heteroatoms independently chosen from N, O, and S, wherein

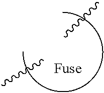

is substituted with 1, 2, 3, or 4 substituents selected from halogen, C₁-C₆alkyl, C₁-C₆haloalkyl, —OR³⁰, and —N(R³⁰)₂; and
all other variables are as defined herein.

In one embodiment, a 3- to 8-membered carbocycle is a 4- to 8-membered carbocycle. In another embodiment a 3- to 8-membered carbocycle is a 4- to 8-membered carbocycle.

In one embodiment, R⁷ and R⁹ are taken together with the atoms to which they are attached to form a 4- to 8-membered carbocycle or a 4- to 8-membered heterocycle containing 1 or 2 heteroatoms independently chosen from N, O, and S;

In one embodiment, R⁹ and R¹¹ are taken together with the atoms to which they are attached to form a 4- to 8-membered carbocycle or a 4- to 8-membered heterocycle containing 1 or 2 heteroatoms independently chosen from N, O, and S;

In one embodiment,

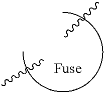

is a 4- to 8-membered carbocycle or a 4- to 8-membered heterocycle containing 1 or 2 heteroatoms independently chosen from N, O, and S; In another aspect, the compound of Formula XIV is selected from:

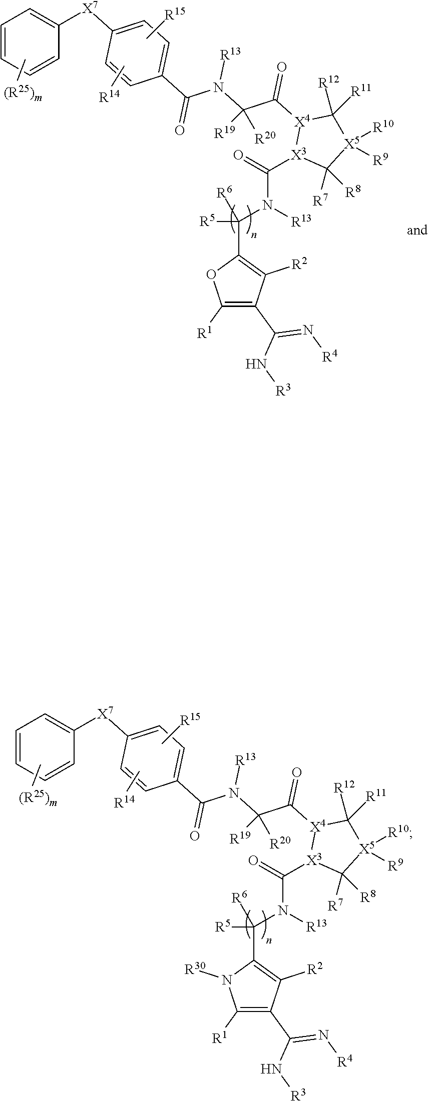

or a pharmaceutically acceptable salt, prodrug, or isolated isomer thereof, optionally in pharmaceutically acceptable carrier; wherein all variables are as defined herein.

In another aspect, the compound of Formula XIV is selected from:

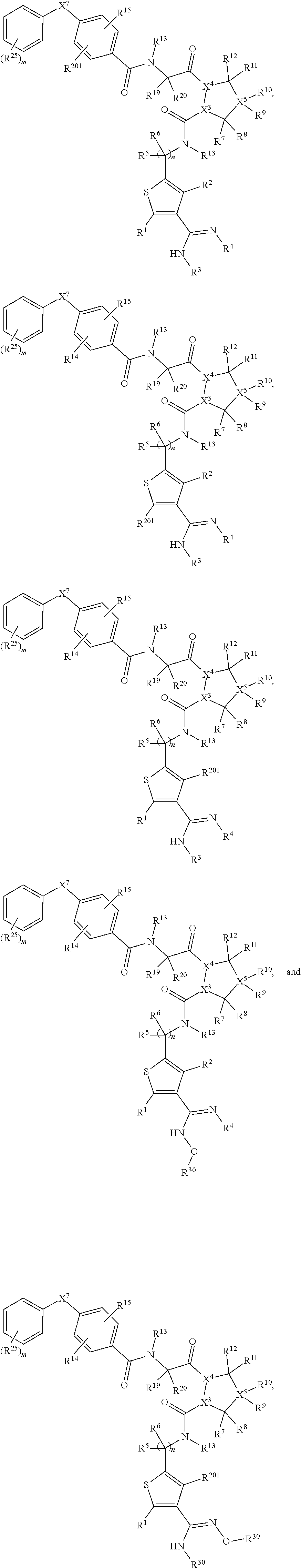

or a pharmaceutically acceptable salt, prodrug, or isolated isomer thereof, optionally in pharmaceutically acceptable carrier;
wherein:
R²⁰¹ is selected from halogen, C₁-C₆alkyl, C₂-C₆alkenyl, C₂-C₆alkynyl, C₁-C₆haloalkyl, —OR³⁰, —SR³⁰, —N(R³⁰)₂, —C(O)R³¹, —S(O)R³¹, —S(O)₂R³¹, heterocycle, aryl, and heteroaryl, each of which R²⁰¹ groups other than halogen are optionally substituted with 1, 2, 3, or 4 substituents independently selected from C₁-C₆alkyl, C₁-C₆alkenyl, C₂-C₆alkynyl, halogen, C₁-C₆haloalkyl, —OR³⁰, —SR³⁰, —N(R³⁰)₂, —C(O)R³¹, —S(O)R³¹, —S(O)₂R³¹, heterocycle, aryl, heteroaryl, cyano, and nitro; and
all other variables are as defined herein.

In certain aspects of the disclosure, R⁹ and R¹¹ are taken together with the atoms to which they are attached to form a cyclopropane.

In one embodiment, the compound of the present disclosure is selected from:

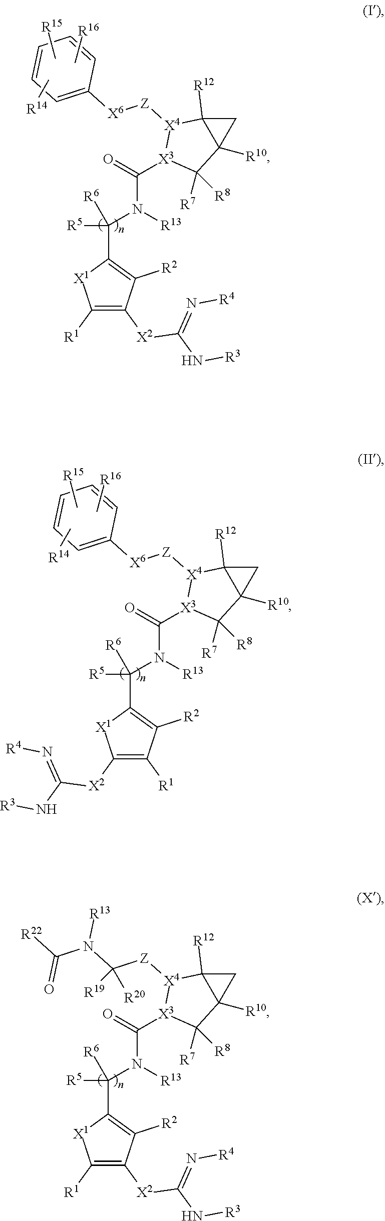

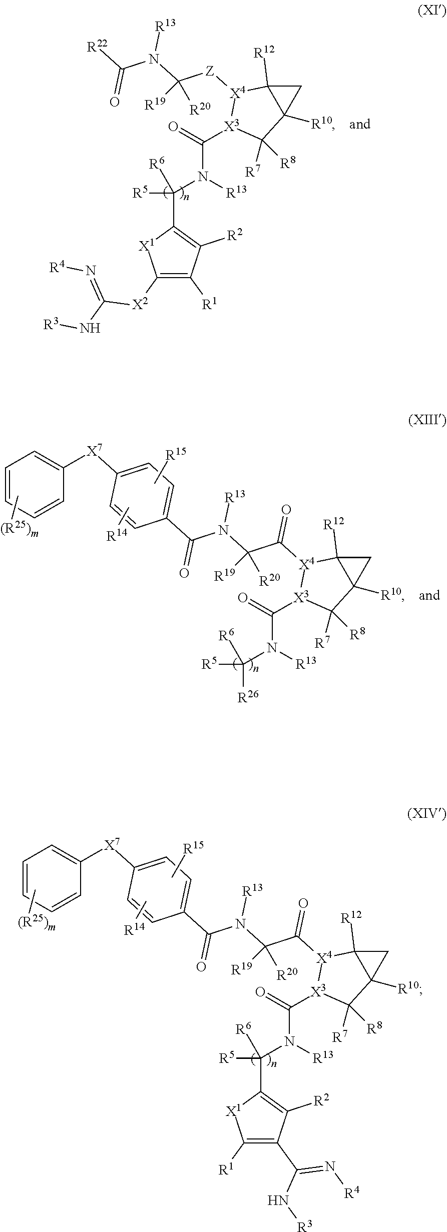

or a pharmaceutically acceptable salt thereof.

In certain embodiments, the compound of the present disclosure is selected from:

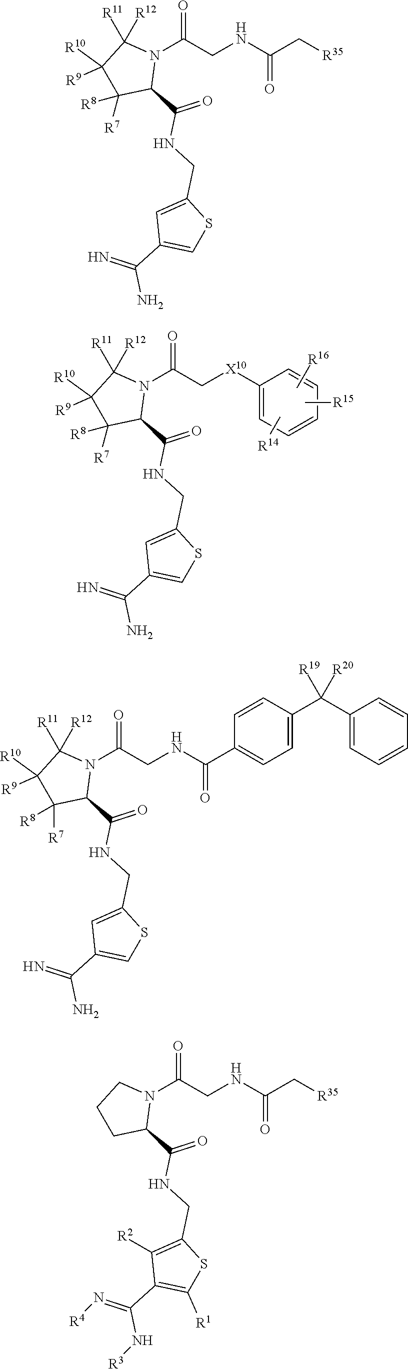

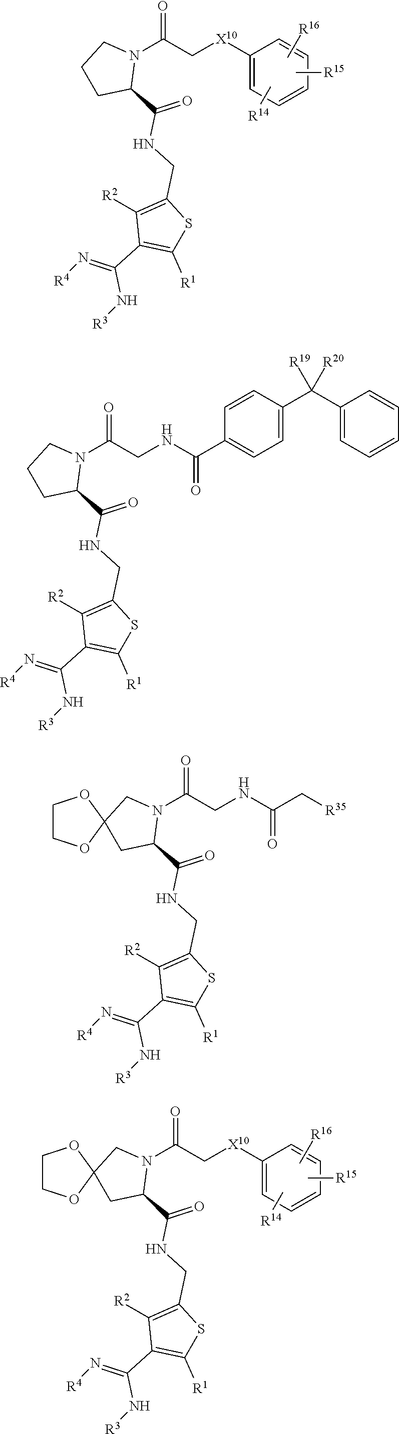

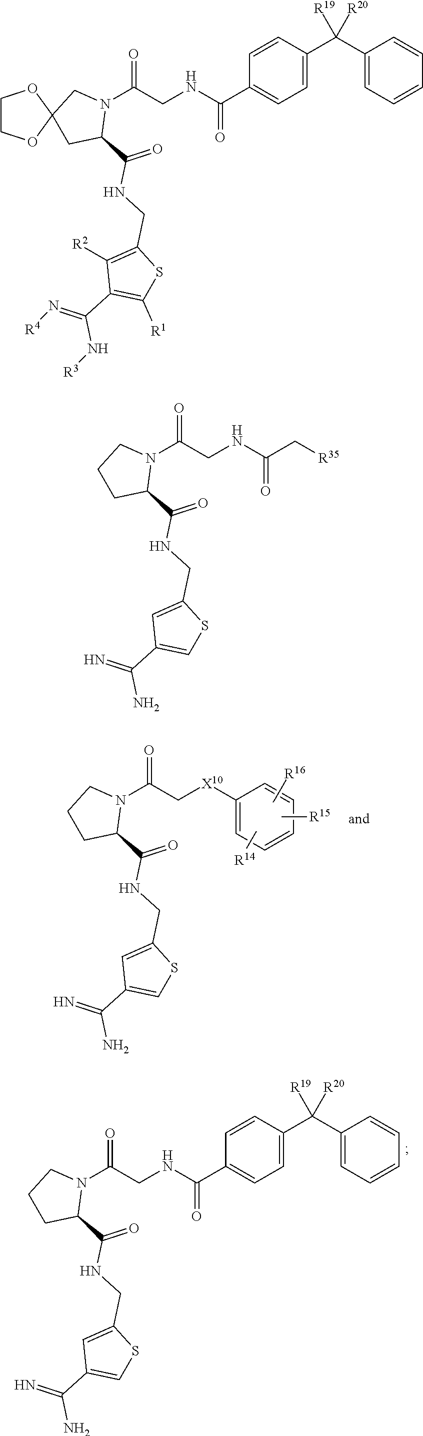

or a pharmaceutically acceptable salt thereof.

In certain embodiments, the compound of the present disclosure is selected from:

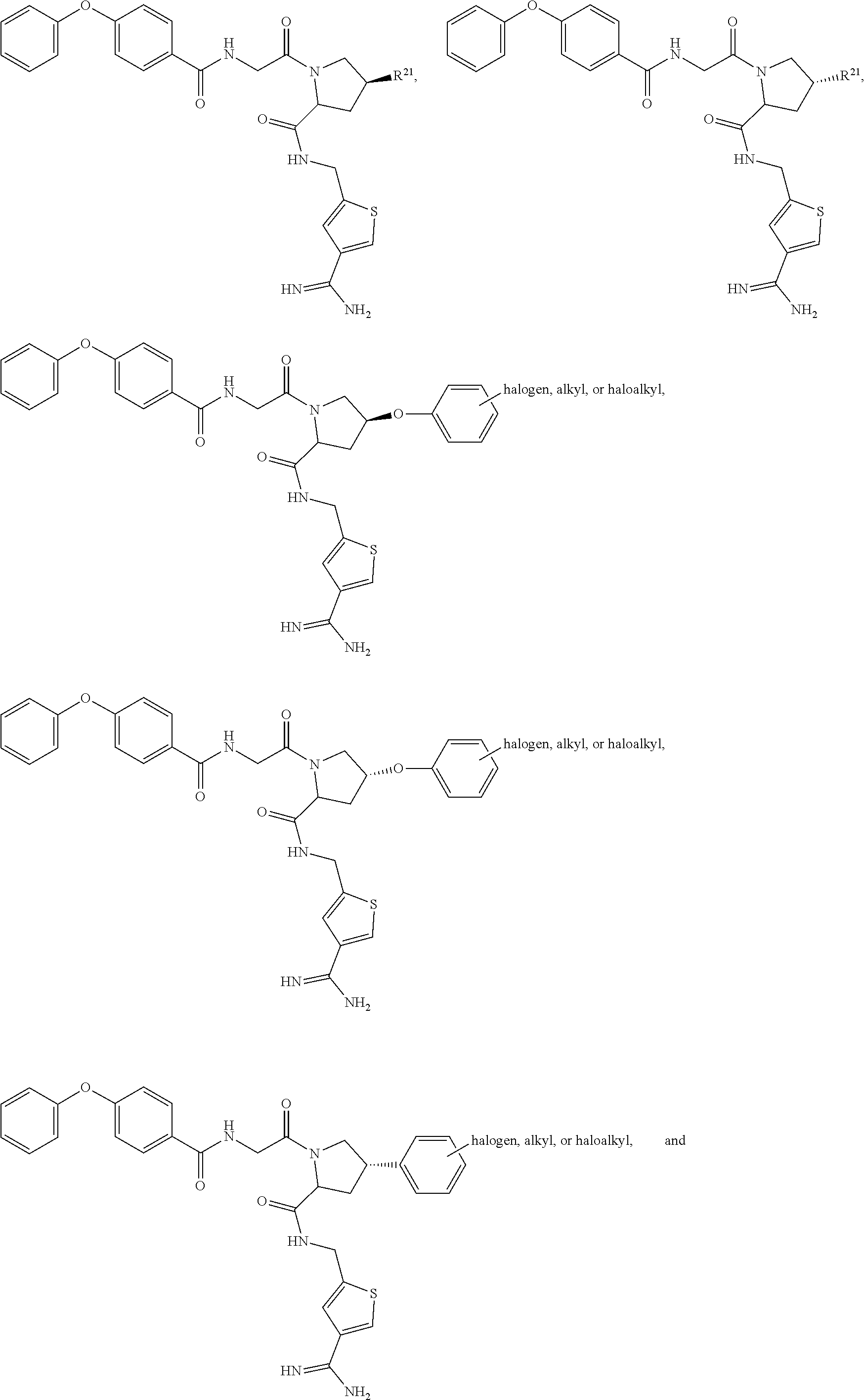

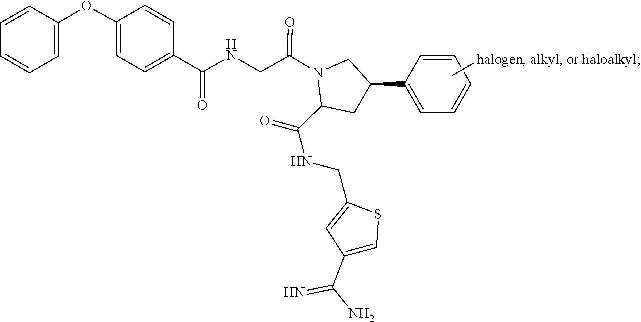

or a pharmaceutically acceptable salt thereof.

In certain embodiments, the compound of the present disclosure is selected from:

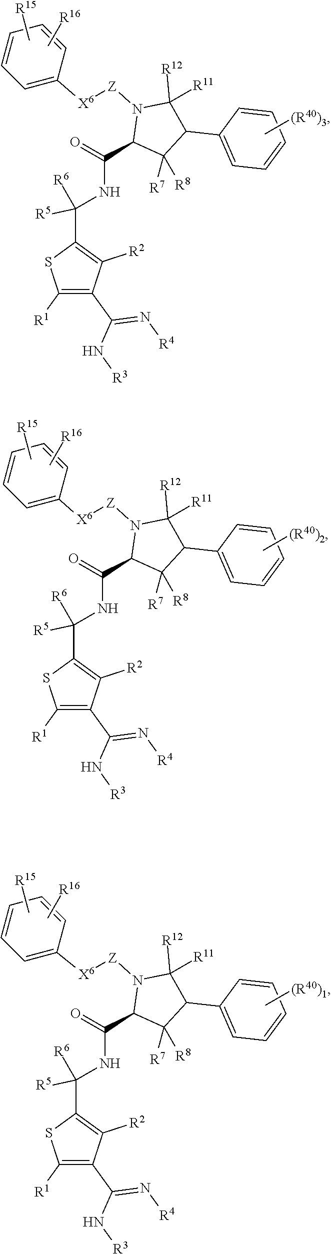

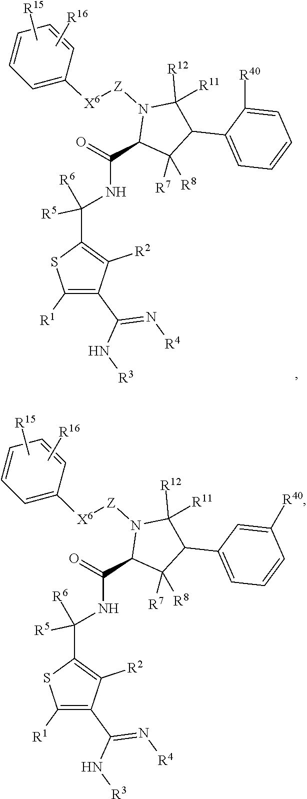

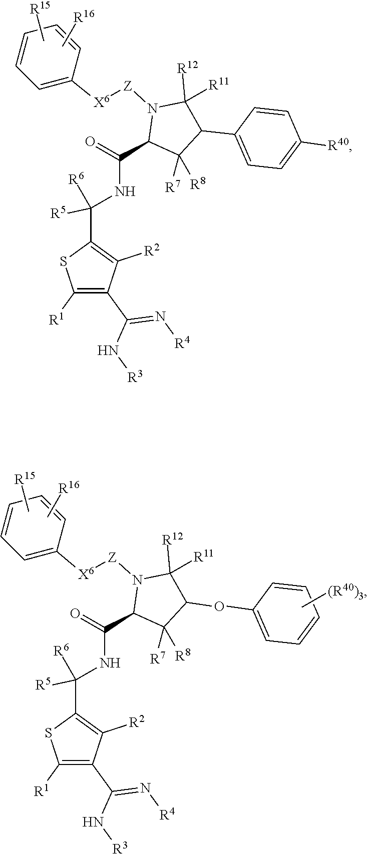

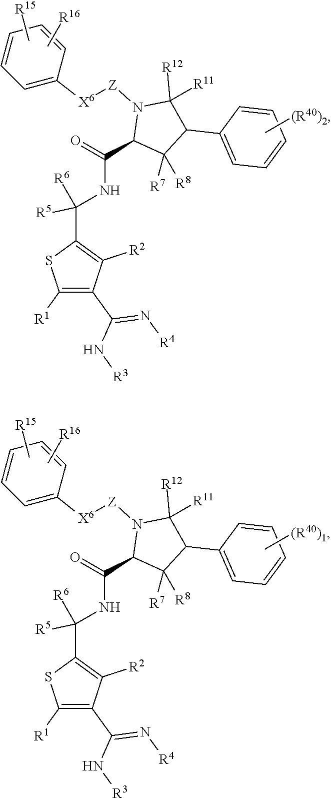

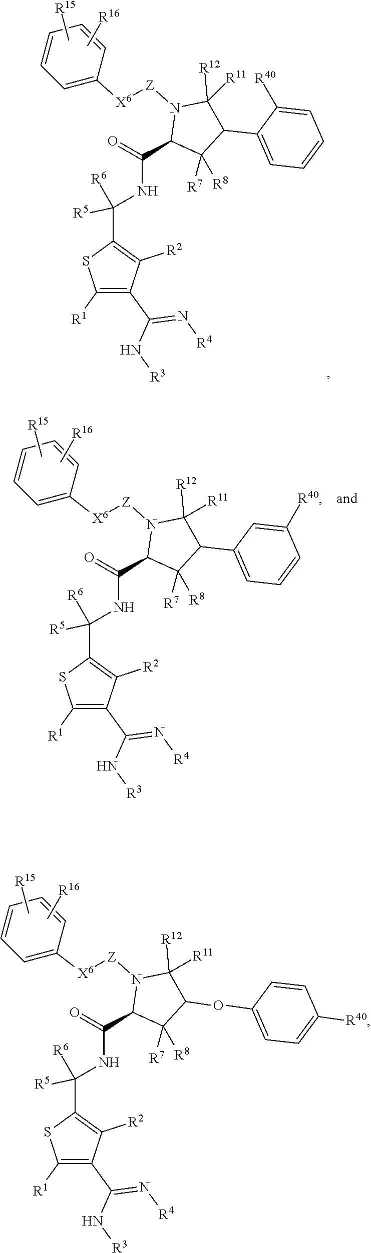

or a pharmaceutically acceptable salt thereof;
wherein each R⁴⁰ is independently selected from: SF₅, C₁-C₆alkyl, C₂-C₆alkenyl, C₂-C₆alkynyl, halogen, C₁-C₆haloalkyl, —OR³⁰, —SR³⁰, —N(R³⁰)₂, —C(O)R³¹, —S(O)R³¹, —S(O)₂R³¹, heterocycle, aryl, heteroaryl, cyano, and nitro.

In certain embodiments, the compound of the present disclosure is selected from:

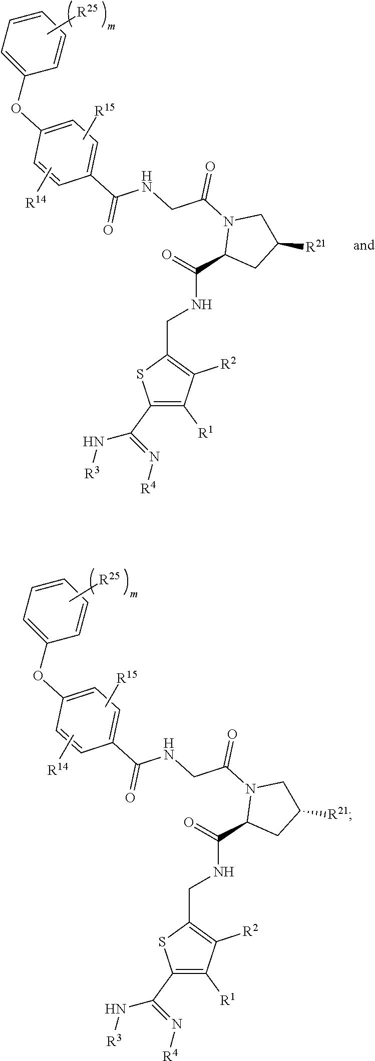

or a pharmaceutically acceptable salt thereof. In certain embodiments, the compound of the present disclosure is:

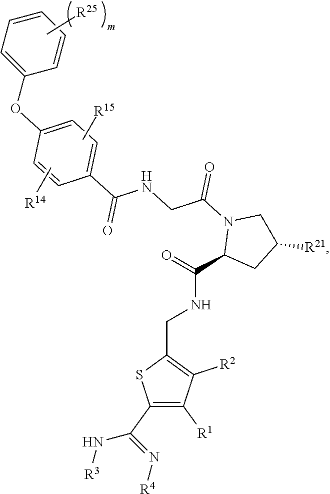

or a pharmaceutically acceptable salt thereof.

In certain embodiments, the compound of the present disclosure is selected from:

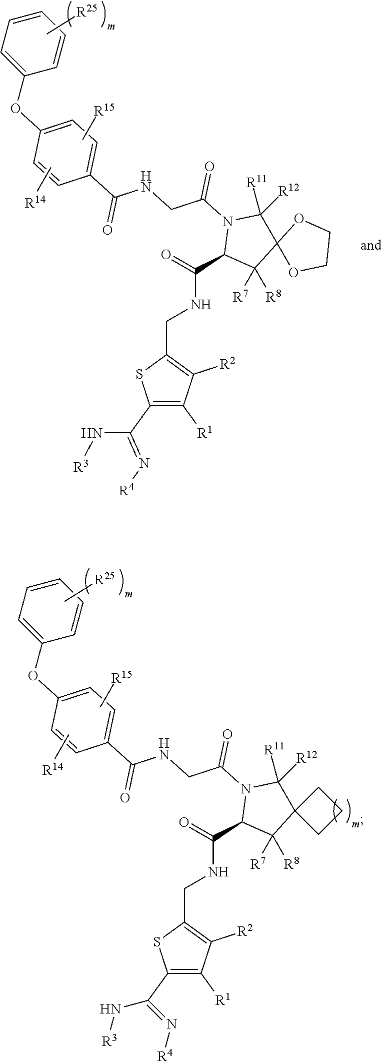

or a pharmaceutically acceptable salt thereof.

### NUMBERED EMBODIMENTS

1. A compound selected from:

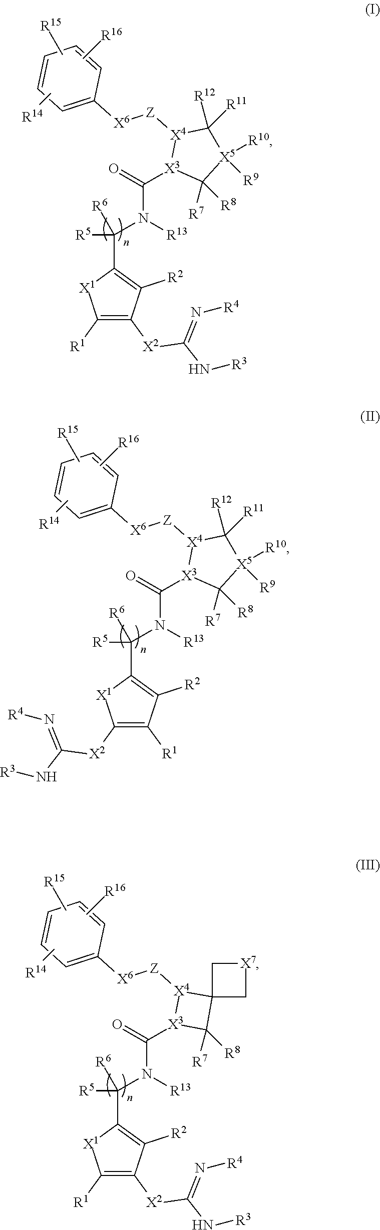

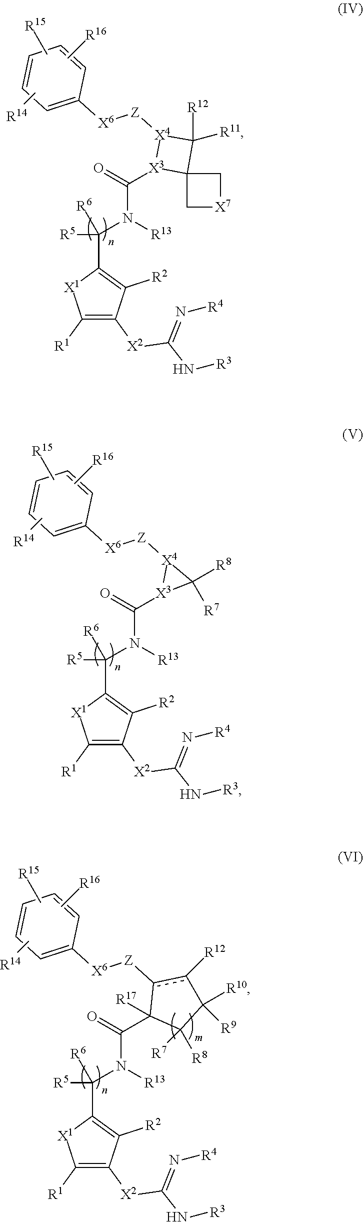

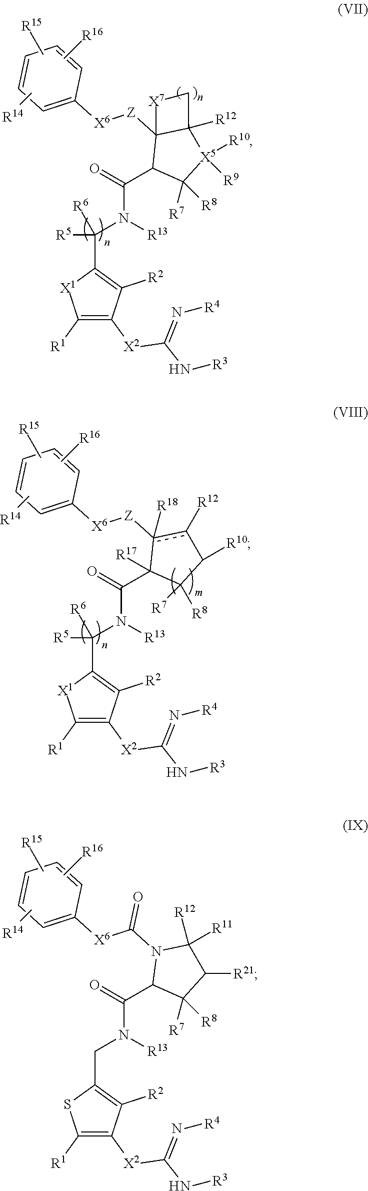

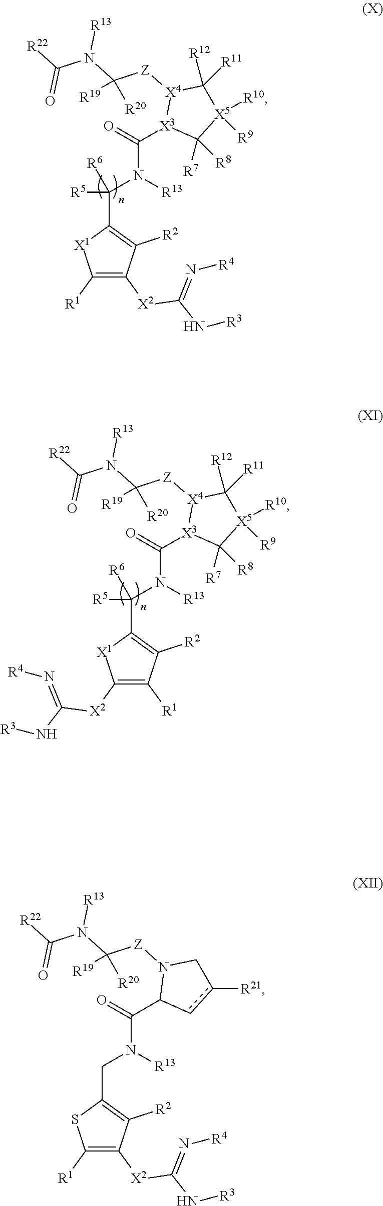

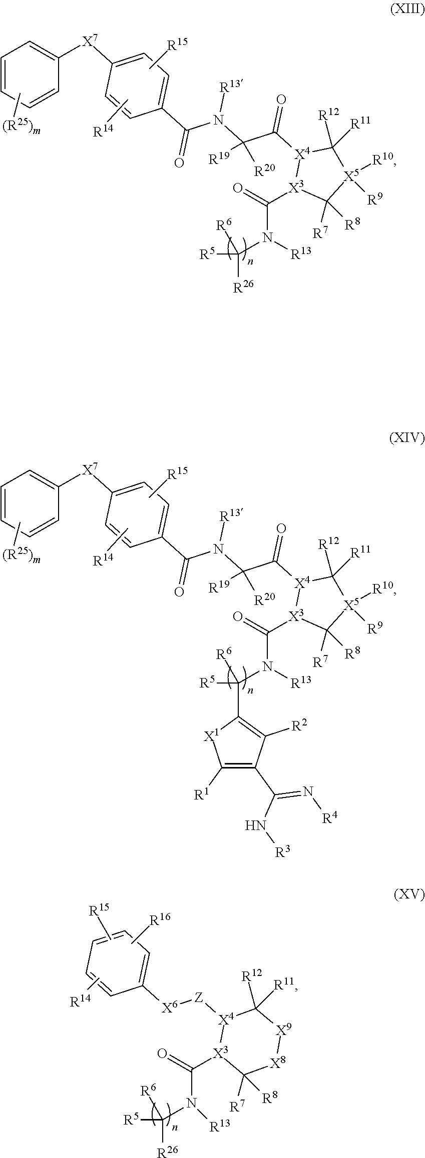

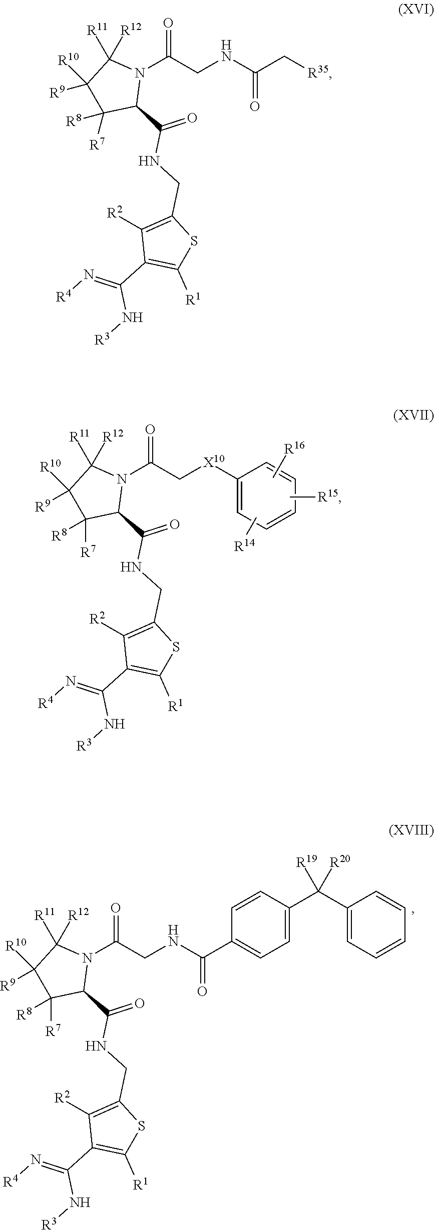

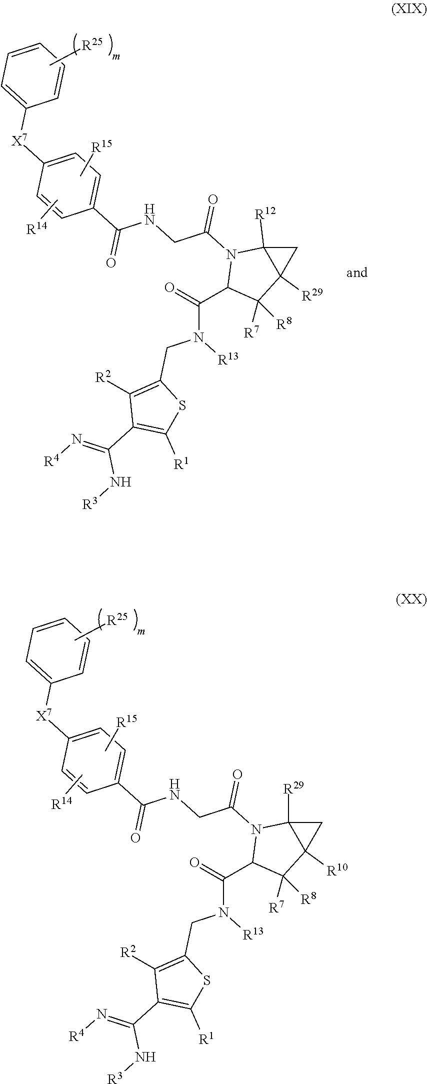

or a pharmaceutically acceptable salt, prodrug, or isolated isomer thereof;
wherein:
each n is independently 0, 1, 2, or 3;
each m is independently 0, 1, 2, or 3;
o is 0, 1, or 2;

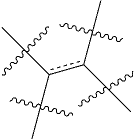

is either a single or a double bond;
Z is CH₂, C(CH₂), or C(O);
X¹ is selected from S, O, and N(R³⁰);
X² is selected from bond, N(R³⁰), and —O—N(R³⁰)—;
X³ is selected from N and C(R¹⁷);
X⁴ is selected from N and C(R¹⁸);
wherein only one of X³ and X⁴ can be N;
X⁵ is C, Si, or S;
X⁶ is selected from

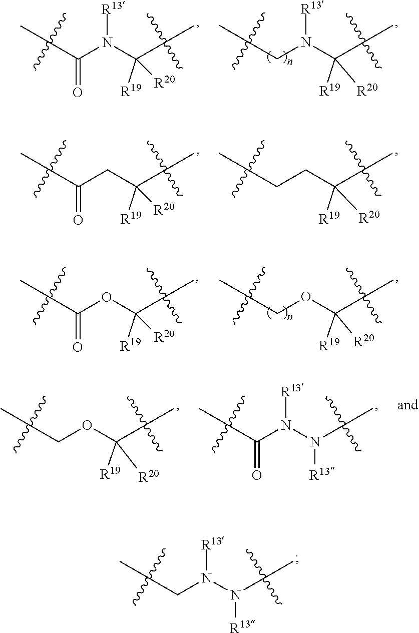

X⁷ is selected from O, S, N(R³⁰), and CR⁵R⁶;
each X⁸ and X⁹ is independently selected from O, S, NR³⁰, CR⁹R¹⁰, CR⁵R⁶. and CH₂; wherein X⁸ and X⁹ cannot both be the same group;
X¹⁰ is selected from

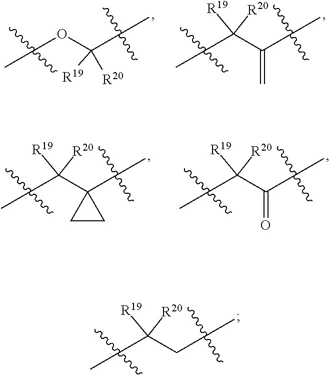

X¹¹ is selected from N and CR¹;
X¹² is selected from N and CR²;
R¹ and R² are independently selected from hydrogen, halogen, C₁-C₆alkyl, C₂-C₆alkenyl, C₂-C₆alkynyl, C₁-C₆haloalkyl, —OR³⁰, —SR³⁰, —N(R³⁰)₂, —C(O)R³¹, —S(O)R³¹, —S(O)₂R³¹, heterocycle, aryl, heteroaryl, cyano, and nitro; each of which R¹ and R² groups other than hydrogen, halogen, cyano, and nitro are optionally substituted with 1, 2, 3, or 4 substituents independently selected from C₁-C₆alkyl, C₂-C₆alkenyl, C₂-C₆alkynyl, halogen, C₁-C₆haloalkyl, —OR³⁰, —SR³⁰, —N(R³⁰)₂, —C(O)R³¹, —S(O)R³¹, —S(O)₂R³¹, heterocycle, aryl, heteroaryl, cyano, and nitro;
R³ and R⁴ are independently selected from hydrogen, nitro, —S(O)₂R³¹, CN, C(O)R³¹, —SR³⁰, and —OR³⁰;
or R³ and R⁴ are instead combined to form a dihydrooxadiazole optionally substituted with 1, 2, or 3 substituents independently selected from C₁-C₆alkyl, C₁-C₆haloalkyl, —OR³⁰, and oxo; an oxadiazole optionally substituted with 1, 2, or 3 substituents independently selected from C₁-C₆alkyl, C₁-C₆haloalkyl, and —OR³⁰; an imidazole optionally substituted with 1, 2, or 3 substituents independently selected from C₁-C₆alkyl, C₁-C₆haloalkyl, and —OR³⁰; or a dihydroimidazole optionally substituted 1, 2, or 3 substituents independently selected from C₁-C₆alkyl, C₁-C₆haloalkyl, —OR³⁰, and oxo;
R⁵ and R⁶ are each independently selected from hydrogen, halogen, C₁-C₆alkyl, C₁-C₆haloalkyl, C₁-C₆hydroxyalky, —OR³⁰, —N(R³⁰)₂, and C(O)R³¹, wherein, when on carbons adjacent to each other, a R⁵ and a R⁶ group may optionally be replaced by a carbon-carbon double bond; or, when n is 1, R⁵ and R⁶, together with the carbon to which they are attached, are replaced with —SO₂—;
or R⁵ and R⁶, together with the carbon atom to which they are attached, combine to form cyclopropyl;
R⁷, R⁸, R⁹, R¹⁰, R¹¹, and R¹² are independently selected from hydrogen, halogen, C₁-C₆alkyl, C₂-C₆alkenyl, C₂-C₆alkynyl, C₁-C₆haloalkyl, —OR³⁰, —SR³⁰, —N(R³⁰)₂, —C(O)R³¹, —S(O)R³¹, —S(O)₂R³¹, —S(O)(NR³¹)(R³¹), carbocycle, heterocycle, aryl, and heteroaryl, each of which R⁷, R⁸, R⁹, R¹⁰, R¹¹, and R¹² groups other than hydrogen and halogen are optionally substituted with 1, 2, 3, or 4 substituents independently selected from C₁-C₆alkyl, C₂-C₆alkenyl, C₂-C₆alkynyl, halogen, C₁-C₆haloalkyl, —OR³⁰, —SR³⁰, —N(R³⁰)₂, —C(O)R³¹, —S(O)R³¹, —S(O)₂R³¹, heterocycle, aryl, heteroaryl, cyano, nitro, and azido;
or R⁷ and R⁸ may be taken together with the carbon to which they are attached to form a 3- to 6-membered carbocyclic spiro ring or a 4- to 6-membered heterocyclic spiro ring containing 1 or 2 heteroatoms independently chosen from N, O, and S, wherein the carbocyclic spiro ring and heterocyclic spiro ring are optionally substituted with one or more halogen, C₁-C₆alkyl, C₁-C₆ haloalkyl, —OR³⁰, —SR³⁰, or —N(R³⁰)₂;
or R⁷ and R⁸ may be taken together with the carbon to which they are attached to form

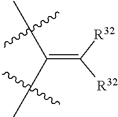

or carbonyl;
or R⁹ and R¹⁰ may be taken together with the atom to which they are attached to form a 3- to 6-membered carbocyclic spiro ring or a 4- to 6-membered heterocyclic spiro ring containing 1 or 2 heteroatoms independently chosen from N, O, and S, wherein the carbocyclic spiro ring and heterocyclic spiro ring are optionally substituted with one or more halogen, C₁-C₆alkyl, C₁-C₆ haloalkyl, —OR³⁰, —SR³⁰, or —N(R³⁰)₂;
or R⁹ and R¹⁰ may be taken together with the atom to which they are attached to form

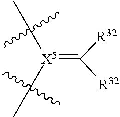

or carbonyl;
or R¹¹ and R¹² may be taken together with the carbon to which they are attached to form a 3- to 6-membered carbocyclic spiro ring or a 4- to 6-membered heterocyclic spiro ring containing 1 or 2 heteroatoms independently chosen from N, O, and S, wherein the carbocyclic spiro ring and heterocyclic spiro ring are optionally substituted with one or more halogen, C₁-C₆alkyl, C₁-C₆ haloalkyl, —OR³⁰, —SR³⁰, or —N(R³⁰)₂;
or R¹¹ and R¹² may be taken together with the carbon to which they are attached to form

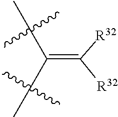

or carbonyl;
or R¹ and R⁹ are taken together with the atoms to which they are attached to form a 3- to 8-membered carbocycle or a 4- to 8-membered heterocycle containing 1 or 2 heteroatoms independently chosen from N, O, and S;
or R⁹ and R¹¹ are taken together with the atoms to which they are attached to form a 3- to 8-membered carbocycle or a 4- to 8-membered heterocycle containing 1 or 2 heteroatoms independently chosen from N, O, and S;
or R⁷ and R¹¹ are taken together with the atoms to which they are attached to form a 1 or 2 carbon bridge;
or, when X⁵ is S, R⁹ and R¹⁰ are absent; each R¹³ is independently selected from hydrogen, C₁-C₆alkyl, and OH;
or R¹³ and R²⁶, together with the atoms to which they are attached, form a heterocycle optionally substituted with R²⁷;
or R¹³, together with the nitrogen atom to which it is attached, is replaced with —O—;
each R^{13′} and R^{13″} is independently selected from hydrogen and C₁-C₆alkyl;
or R^{13′} and R¹⁴, together with the atoms to which they are attached, combine to form a 5- or 6-membered heterocycle containing one N;
R¹⁴, R¹⁵, and R¹⁶ are independently selected from hydrogen, halogen, SF₅, C₁-C₆alkyl, C₂-C₆alkenyl, C₂-C₆alkynyl, C₁-C₆haloalkyl, —C₁-C₆alkyl-aryl. —OR³⁰, —SR³⁰, —N(R³⁰)₂, —C(O)R³¹, —S(O)R³¹, —S(O)₂R³¹, heterocycle, aryl, heteroaryl, cyano, and nitro each of which R¹⁴, R¹⁵, and R¹⁶ groups other than hydrogen, halogen, cyano, and nitro are optionally substituted with 1, 2, 3, or 4 substituents independently selected from SF₅, C₁-C₆alkyl, C₂-C₆alkenyl, C₂-C₆alkynyl, halogen, C₁-C₆haloalkyl, —OR³⁰, —SR³⁰, —N(R³⁰)₂, —C(O)R³¹, —S(O)R³¹, —S(O)₂R³¹, heterocycle, aryl, heteroaryl, cyano, and nitro;
R¹⁷ and R¹⁸ are independently selected from hydrogen, halogen, C₁-C₆alkyl, C₁-C₆haloalkyl, —OR³⁰, and —N(R³⁰)₂;
or R¹⁷ and R¹⁸ are taken together with the carbons to which they are attached to form a double bond;
R¹⁹ and R²⁰ are independently selected from hydrogen, C₁-C₆alkyl, C₅-C₁₀ bicyclic carbocycle, C₄-C₆heterocycle, halogen, C₁-C₆haloalkyl, —OR³⁰, —N(R³⁰)₂, —(CH₂)ₙ—R³³, and

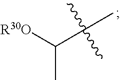

R²¹ is selected from C₁-C₆haloalkyl, —O—C₁-C₆haloalkyl, C₁-C₆alkyl, —O—C₁-C₆alkyl, —S(O)(NR³¹)R³¹, carbocycle, aryl, —O-aryl, heteroaryl, —O-carbocycle, or —O-heteroaryl, each of which R²¹ group is optionally substituted with 1, 2, 3, or 4 substituents independently selected from SF₅, C₁-C₆alkyl, C₂-C₆alkenyl, C₂-C₆alkynyl, halogen, C₁-C₆haloalkyl, —OR³⁰, —SR³⁰, —N(R³⁰)₂, —C(O)R³¹, —S(O)R³¹, —S(O)₂R³¹, heterocycle, aryl, heteroaryl, cyano, nitro, and azido;
R²² is selected from —C₁-C₆alkyl-R²³, —C₂-C₆alkenyl-R²³, —C₂-C₆alkynyl-R²³, -heteroaryl-R²³, -carbocycle-R²³, and bicyclic cycloalkyl-R²³, each of which R²² is optionally substituted with 1, 2, 3, or 4 substituents independently selected from C₁-C₆alkyl, C₂-C₆alkenyl, C₂-C₆alkynyl, halogen, C₁-C₆haloalkyl,
   —OR³⁰, —SR³⁰, —N(R³⁰)₂, —C(O)R³¹, —S(O)R³¹, —S(O)₂R³¹, heterocycle, aryl, heteroaryl, cyano, and nitro;
R²³ is selected from hydrogen, sugar, —OR³⁰, —SR³⁰, —N(R³⁰)₂, —C(O)R³¹, and —S(O)R³¹, —S(O)₂R³¹;
each R²⁵ is independently selected from hydrogen, SF₅, halogen, C₁-C₆alkyl, C₂-C₆alkenyl, C₂-C₆alkynyl, C₁-C₆haloalkyl, —OR³⁰, —SR³⁰, —N(R³⁰)₂, —C(O)R³¹, —S(O)R³¹, —S(O)₂R³¹, —S(O)(NR³¹)R³¹, —P(O)(OR³¹)R³¹, heterocycle, aryl, heteroaryl, cyano, and nitro; each of which R²⁵ groups other than hydrogen, halogen, cyano, and nitro are optionally substituted with 1, 2, 3, or 4 substituents independently selected from C₁-C₆alkyl, C₂-C₆alkenyl, C₂-C₆alkynyl, halogen, C₁-C₆haloalkyl, —OR³⁰, —SR³⁰, —N(R³⁰)₂,
   —C(O)R³¹, —S(O)R³¹, —S(O)₂R³¹, heterocycle, aryl, heteroaryl, cyano, and nitro;
R²⁶ is selected from

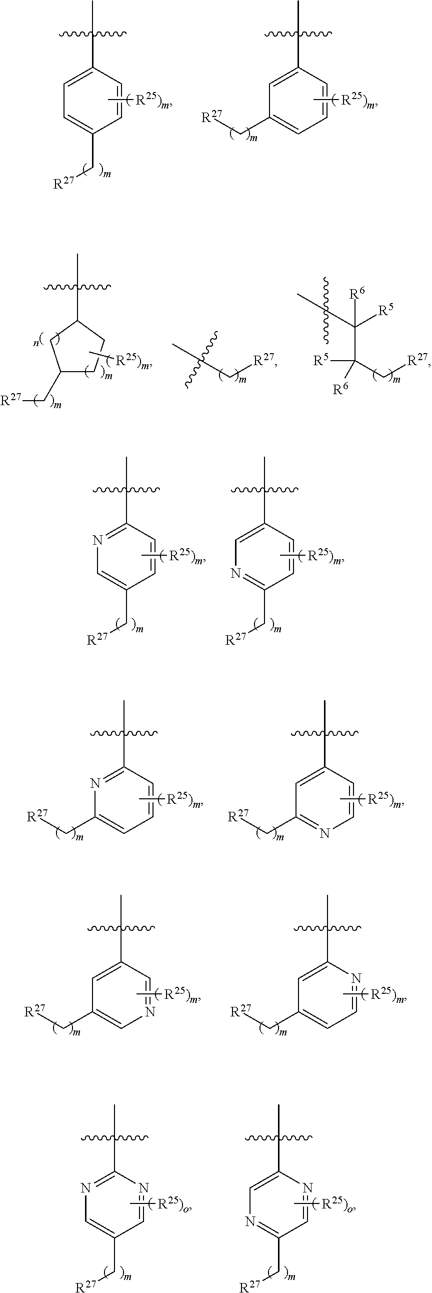

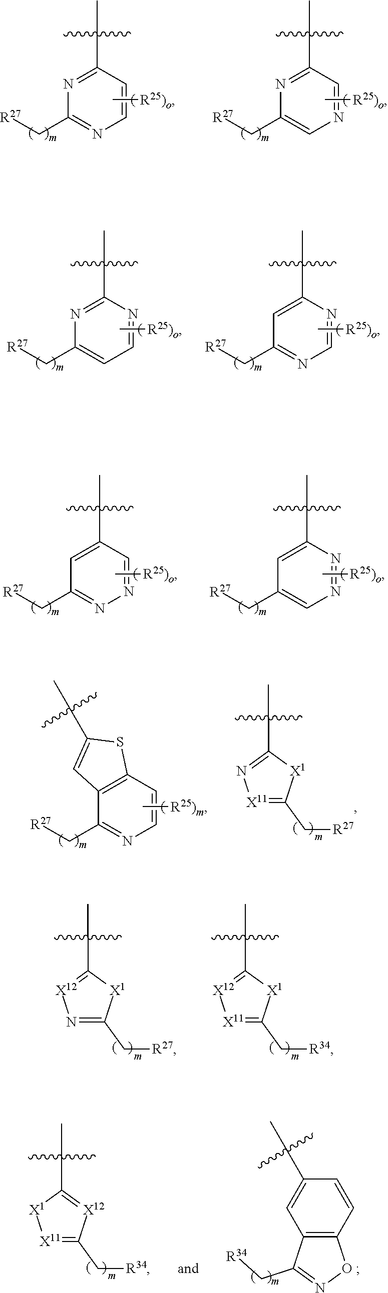

R²⁷ is selected from —OR³⁰, S-methylsulfonimidoyl,

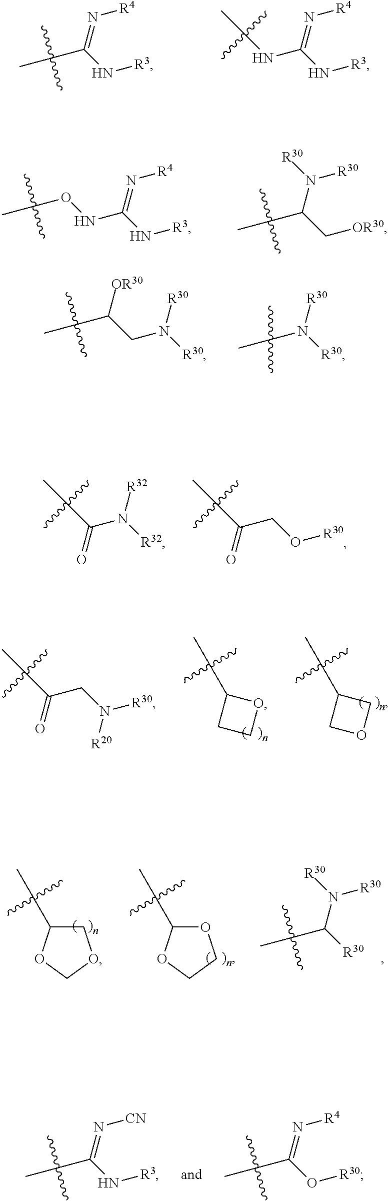

R²⁹ is selected from halogen, C₁-C₆alkyl, C₂-C₆alkenyl, C₂-C₆alkynyl, C₁-C₆haloalkyl, —OR³⁰, —SR³⁰, —N(R³⁰)₂, —C(O)R³¹, —S(O)R³¹, —S(O)₂R³¹, heterocycle, aryl, and heteroaryl, each of which R²⁹ groups other than hydrogen and halogen are optionally substituted with 1, 2, 3, or 4 substituents independently selected from C₁-C₆alkyl, C₂-C₆alkenyl, C₂-C₆alkynyl, halogen, C₁-C₆haloalkyl, —OR³⁰, —SR³⁰, —N(R³⁰)₂, —C(O)R³¹, —S(O)R³¹, —S(O)₂R³¹, heterocycle, aryl, heteroaryl, cyano, and nitro.
each R³⁰ is independently selected from hydrogen, C₁-C₆alkyl, C₁-C₆haloalkyl, carbocycle, aryl, heteroaryl, heterocycle, and C(O)R³¹, each R³⁰ other than C(O)R³¹ is optionally substituted with 1, 2, 3, or 4 substituents selected from C₁-C₆alkyl, halogen, SF₅, —C(O)R³¹, —N(R³⁰)₂, aryl, heteroaryl, —OR³², —S(O)(NR³¹)R³¹, and carbocycle;
or R³⁰ and R⁴ in

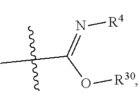

together with the N and O atoms to which each is attached and the carbon atom to which the N and O atoms are attached, combine to form oxadiazole optionally substituted with 1, 2, or 3 substituents independently selected from C₁-C₆alkyl, C₁-C₆haloalkyl, and —OR³⁰;
each R³¹ is independently selected from hydrogen, C₁-C₆alkyl, C₁-C₆haloalkyl, —OR³², —SR³², —N(R³²)₂, heterocycle, aryl, and heteroaryl;
each R³² is independently selected from hydrogen, halogen, C₁-C₆alkyl, and C₁-C₆haloalkyl;
each R³³ is independently selected from hydrogen, guanidine, heteroaryl, aryl, —C₆HS—OR³⁰; —OR³⁰, —SR³⁰, —SeR³⁰, —N(R³⁰)₂, and —C(O)R³¹;
R³⁴ is selected from

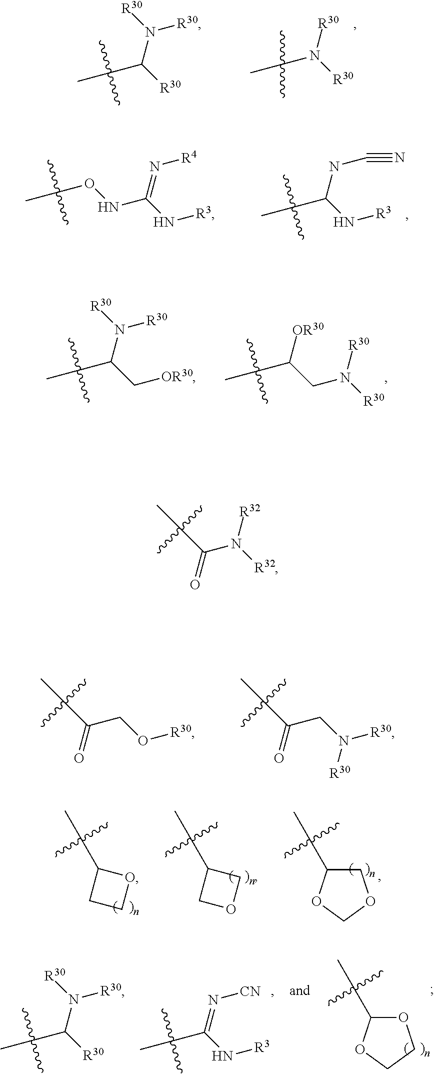

and
R³⁵ is selected from C₃-C₁₀alkyl or C₃-C₁₀haloalkyl.

2. The compound of embodiment 1, wherein for compounds of Formula I and Formula II at least one of the following is satisfied:
a. X³ is C(R¹⁷) and X⁴ is C(R¹⁸);
b. R¹⁷ is selected from halogen, C₁-C₆alkyl, C₁-C₆haloalkyl, —OR³⁰, and —N(R³⁰)₂;
c. X⁵ is Si;
d. X⁵ is S and at least two of R⁷, R⁸, R¹¹, and R¹² are not hydrogen, no more than one of R⁷ and R⁸ is halogen, and no more than one of R¹¹ and R¹² is halogen;
e. Z is C(CH₂);
f. Z is CH₂;
g. R⁷ and R⁸ are taken together with the carbon to which they are attached to form a 3- to 6-membered carbocyclic spiro ring; a 4- to 6-membered heterocyclic spiro ring containing 1 or R³² 2 heteroatoms independently chosen from N, O, and S;

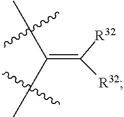

or a carbonyl;
h. R⁹ and R¹⁰ are taken together with the carbon to which they are attached to form a 3- to 6-membered carbocyclic spiro ring; a 4- to 6-membered heterocyclic spiro ring containing 1 or 2 heteroatoms independently chosen from N, O, and S;

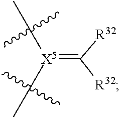

or a carbonyl;
i. R¹¹ and R¹² are taken together with the carbon to which they are attached to form a 3- to 6-membered carbocyclic spiro ring; a 4- to 6-membered heterocyclic spiro ring containing 1 or 2 heteroatoms independently chosen from N, O, and S;

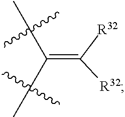

or a carbonyl;
j. R⁷ and R⁹ are taken together with the atoms to which they are attached to form a 3- to 8-membered carbocycle or a 4- to 8-membered heterocycle containing 1 or 2 heteroatoms independently chosen from N, O, and S; and R⁸ or R¹⁰ is not hydrogen;
k. R⁹ and R¹¹ are taken together with the atoms to which they are attached to form a 3- to 8-membered carbocycle or a 4- to 8-membered heterocycle containing 1 or 2 heteroatoms independently chosen from N, O, and S; and R¹⁰ or R¹² is not hydrogen;
l. R⁷ and R¹¹ are taken together with the atoms to which they are attached to form a 1 or 2 carbon bridge;
m. X⁶ is selected from

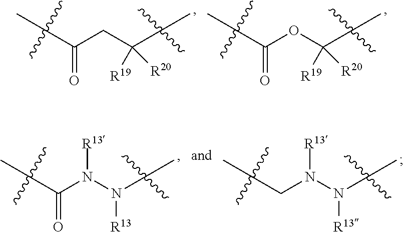

n. at least one of R³ and R⁴ is CN, nitro, —S(O)₂R³¹, —SR³⁰, or C(O)R³¹;
o. R³ and R⁴ are combined to form a dihydroxadiazole optionally substituted with 1, 2, or 3 substituents independently selected from C₁-C₆alkyl, C₁-C₆haloalkyl, —OR³⁰, and oxo; an oxadiazole optionally substituted with 1, 2, or 3 substituents independently selected from C₁-C₆alkyl, C₁-C₆haloalkyl, and —OR³⁰; an imidazole optionally substituted with 1, 2, or 3 substituents independently selected from C₁-C₆alkyl, C₁-C₆haloalkyl, and —OR³⁰; or a dihydroimidazole optionally substituted 1, 2, or 3 substituents independently selected from C₁-C₆alkyl, C₁-C₆haloalkyl, —OR³⁰, and oxo; or
   p. R²⁷ is

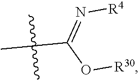

and R³ and R⁴ in

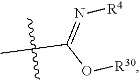

together with the N and O atoms to which each is attached and the carbon atom to which the N and O atoms are attached, combine to form oxadiazole optionally substituted with 1, 2, or 3 substituents independently selected from C₁-C₆alkyl, C₁-C₆haloalkyl, and —OR³⁰;
   wherein for compounds of Formula X and Formula XI at least one of the following is satisfied:
a. X³ is C(R¹⁷) and X⁴ is C(R¹⁸);
b. R¹⁷ is selected from halogen, C₁-C₆alkyl, C₁-C₆haloalkyl, —OR³⁰, and —N(R³⁰)₂;
c. X⁵ is Si;
d. X⁵ is S and at least two of R⁷, R⁸, R¹¹, and R¹² are not hydrogen, no more than one of R⁷ and R⁸ is halogen, and no more than one of R¹¹ and R¹² is halogen;
e. Z is C(CH₂);
f. Z is CH₂;
g. R⁷ and R⁸ are taken together with the carbon to which they are attached to form a 3- to 6-membered carbocyclic spiro ring; a 4- to 6-membered heterocyclic spiro ring containing 1 or 2 heteroatoms independently chosen from N, O, and S;

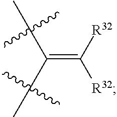

or a carbonyl;
h. R⁹ and R¹⁰ are taken together with the carbon to which they are attached to form a 3- to 6-membered carbocyclic spiro ring; a 4- to 6-membered heterocyclic spiro ring containing 1 or 2 heteroatoms independently chosen from N, O, and S;

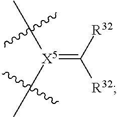

a carbonyl;
i. R⁹ and R¹¹ are taken together with the atoms to which they are attached to form a 3- to 8-membered carbocycle or a 4- to 8-membered heterocycle containing 1 or 2 heteroatoms independently chosen from N, O, and S; and R¹⁰ is not hydrogen;
j. R¹¹ and R¹² are taken together with the carbon to which they are attached to form a 3- to 6-membered carbocyclic spiro ring; a 4- to 6-membered heterocyclic spiro ring containing 1 or 2 heteroatoms independently chosen from N, O, and S;

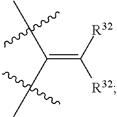

a carbonyl;
k. R⁷ and R⁹ are taken together with the atoms to which they are attached to form a 3- to 8-membered carbocycle or a 4- to 8-membered heterocycle containing 1 or 2 heteroatoms independently chosen from N, O, and S; and R¹⁰ is not hydrogen;
l. R⁷ and R¹¹ are taken together with the atoms to which they are attached to form a 1 or 2 carbon bridge;
m. R²² is substituted with at least three OR³⁰ groups;
n. R²³ is a sugar;
o. at least one of R³ and R⁴ is CN, nitro, —S(O)₂R³¹, —SR³⁰, or C(O)R³¹;
p. R³ and R⁴ are combined to form a dihydroxadiazole optionally substituted with 1, 2, or 3 substituents independently selected from C₁-C₆alkyl, C₁-C₆haloalkyl, —OR³⁰, and oxo; an oxadiazole optionally substituted with 1, 2, or 3 substituents independently selected from C₁-C₆alkyl, C₁-C₆haloalkyl, and —OR³⁰; an imidazole optionally substituted with 1, 2, or 3 substituents independently selected from C₁-C₆alkyl, C₁-C₆haloalkyl, and —OR³⁰; or a dihydroimidazole optionally substituted 1, 2, or 3 substituents independently selected from C₁-C₆alkyl C₁-C₆haloalkyl, —OR³⁰, and oxo; or
q. R²⁷ is

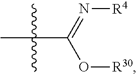

and R³⁰ and R⁴ in

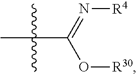

together with the N and O atoms to which each is attached and the carbon atom to which the N and O atoms are attached, combine to form oxadiazole optionally substituted with 1, 2, or 3 substituents independently selected from C₁-C₆alkyl, C₁-C₆haloalkyl, and —OR³⁰;
   wherein for compounds of Formula XIV at least one of the following is satisfied:
a. X¹ is O or N(R³⁰);
b. R¹⁴ is not hydrogen;
c. R¹ is not hydrogen;
d. R² is not hydrogen;
e. R³ is not hydrogen; or
f. R⁴ is not hydrogen. 3. The compound of embodiment 1 or 2, wherein the compound is selected from:

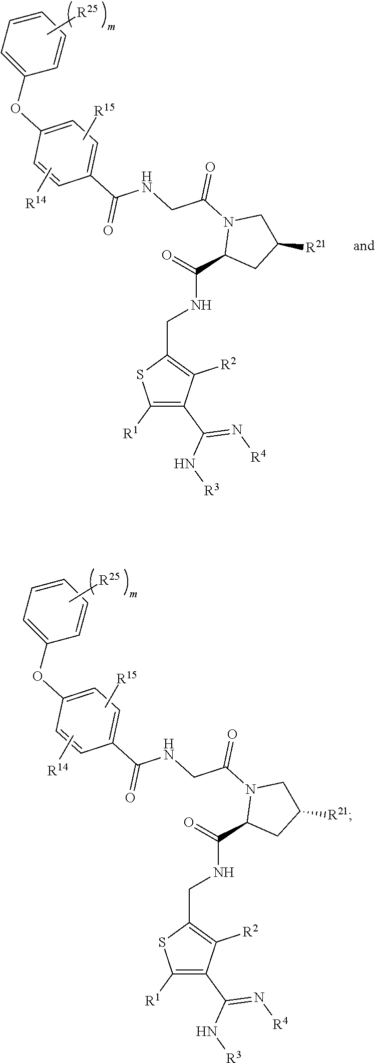

or a pharmaceutically acceptable salt, prodrug, or isolated isomer thereof.

4. The compound of embodiment 1 or 2, wherein the compound is of formula:

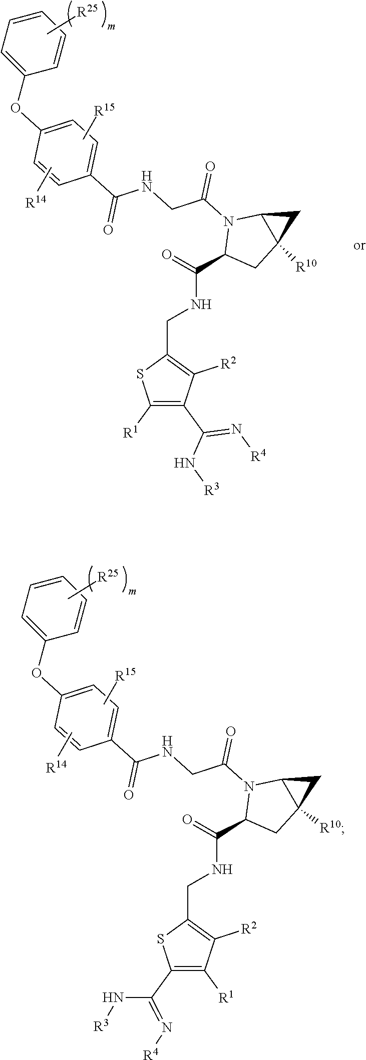

or a pharmaceutically acceptable salt, prodrug, or isolated isomer thereof.

5. The compound of embodiment 1 or 2, of formula:

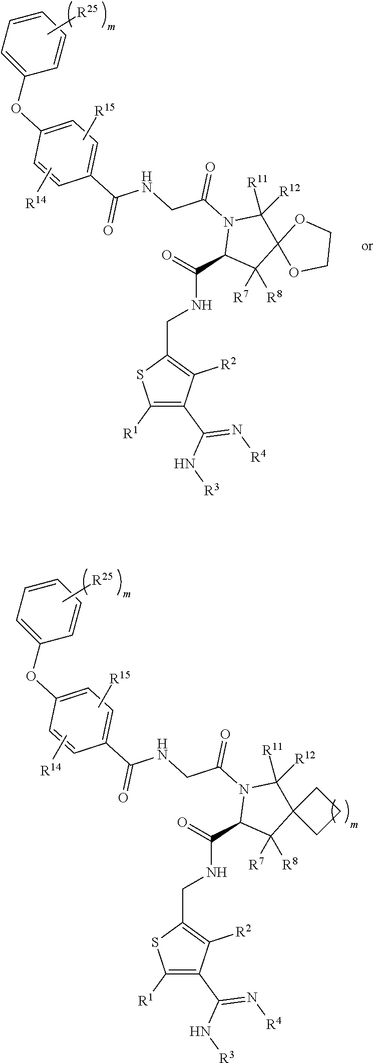

or a pharmaceutically acceptable salt, prodrug, or isolated isomer thereof.

6. The compound of embodiment 1 or 2, selected from:

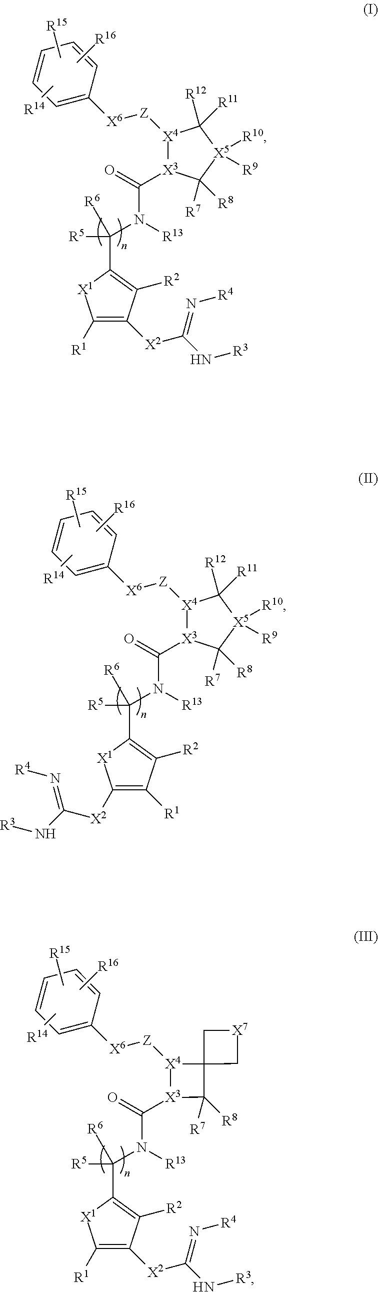

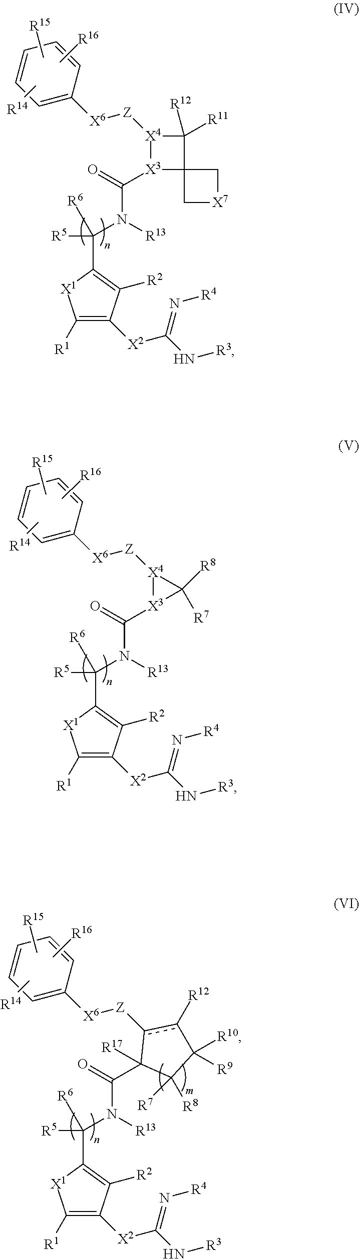

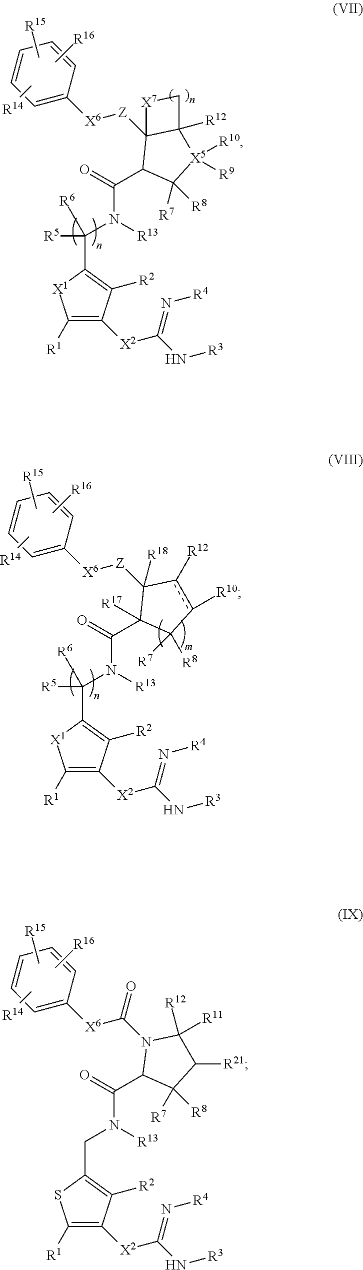

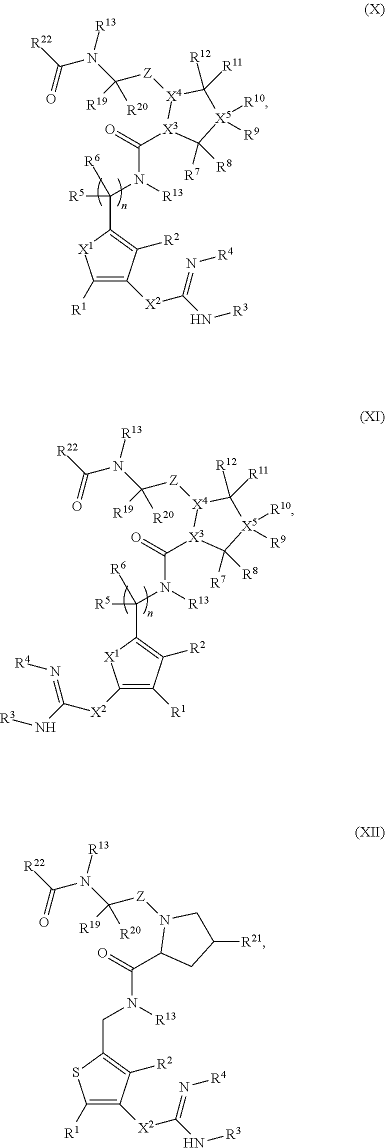

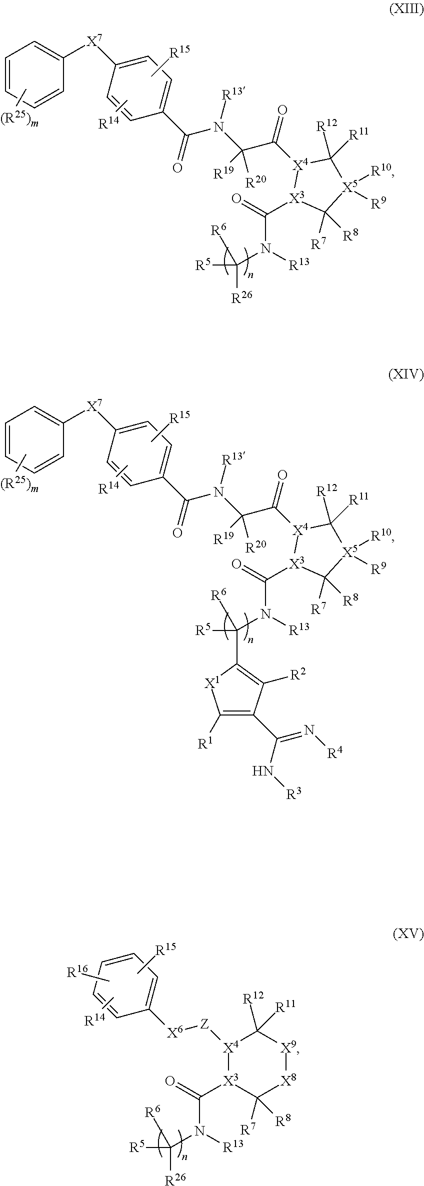

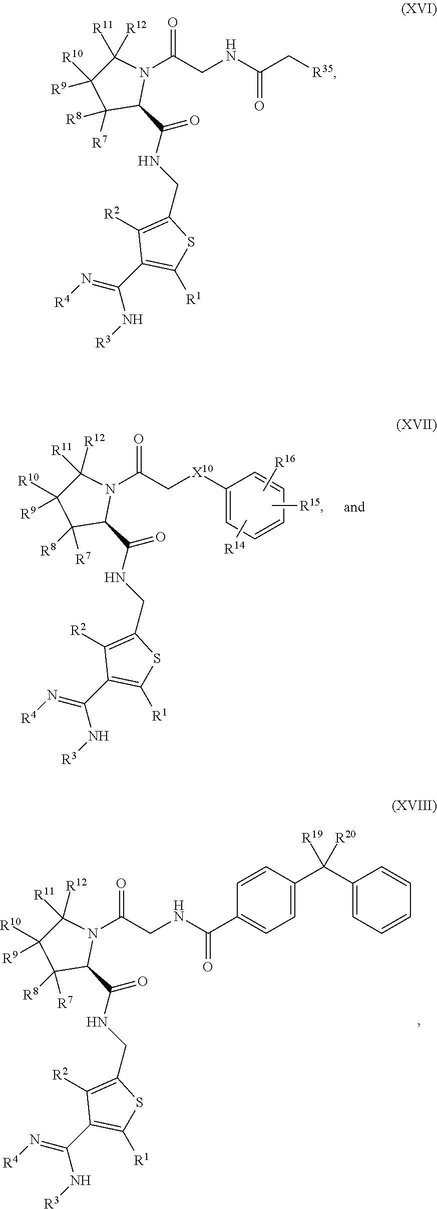

wherein
R²¹ is selected from C₁-C₆alkyl and —O—C₁-C₆alkyl;
each R²⁵ is independently selected from hydrogen, halogen, C₁-C₆alkyl, C₂-C₆alkenyl, C₂-C₆alkynyl, C₁-C₆haloalkyl, —OR³⁰, —SR³⁰, —N(R³⁰)₂, —C(O)R³¹, —S(O)R³¹, —S(O)₂R³¹, heterocycle, aryl, heteroaryl, cyano, and nitro; each of which R¹ and R² groups other than hydrogen, halogen, cyano, and nitro are optionally substituted with 1, 2, 3, or 4 substituents independently selected from C₁-C₆alkyl, C₂-C₆alkenyl, C₂-C₆alkynyl, halogen, C₁-C₆haloalkyl, —OR³⁰, —SR³⁰, —N(R³⁰)₂, —C(O)R³¹, —S(O)R³¹, —S(O)₂R³¹, heterocycle, aryl, heteroaryl, cyano, and nitro;
R¹⁴, R¹⁵, and R¹⁶ are independently selected from hydrogen, halogen, C₁-C₆alkyl, C₂-C₆alkenyl, C₂-C₆alkynyl, C₁-C₆haloalkyl, —C₁-C₆alkyl-aryl, —OR³⁰, —SR³⁰, —N(R³⁰)₂, —C(O)R³¹, —S(O)R³¹, —S(O)₂R³¹, heterocycle, aryl, heteroaryl, cyano, and nitro each of which R¹⁴, R¹⁵, and R¹⁶ groups other than hydrogen, halogen, cyano, and nitro are optionally substituted with 1, 2, 3, or 4 substituents independently selected from C₁-C₆alkyl, C₂-C₆alkenyl, C₂-C₆alkynyl, halogen, C₁-C₆haloalkyl, —OR³⁰, —SR³⁰, —N(R³⁰)₂, —C(O)R³¹, —S(O)R³¹, —S(O)₂R³¹, heterocycle, aryl, heteroaryl, cyano, and nitro.

7. In another embodiment, the compound of the present disclosure is selected from:

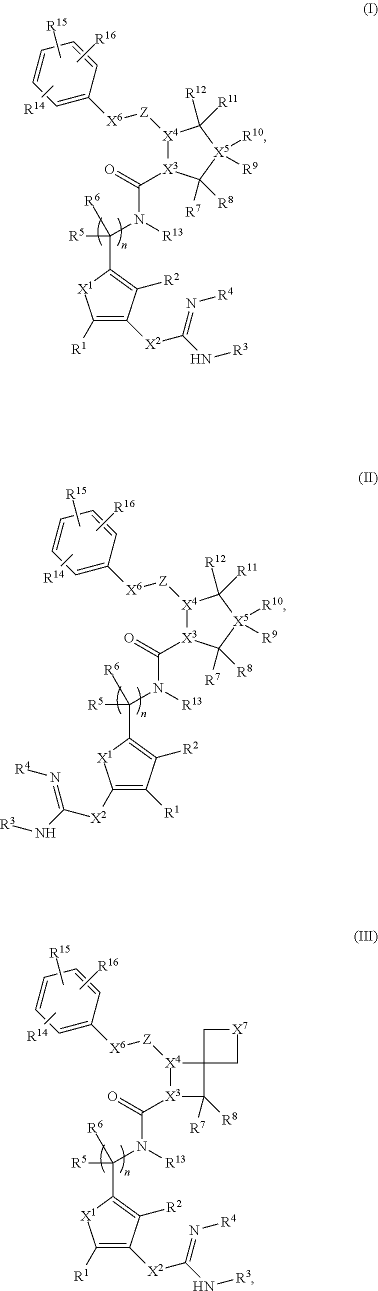

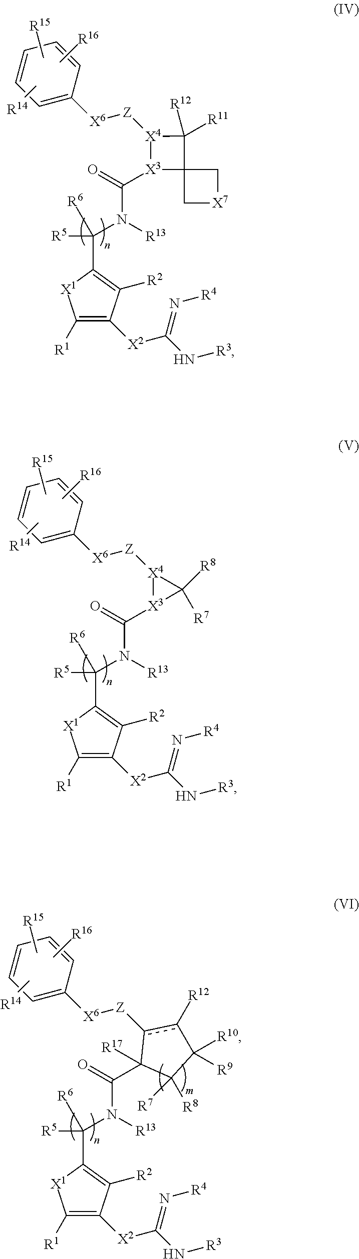

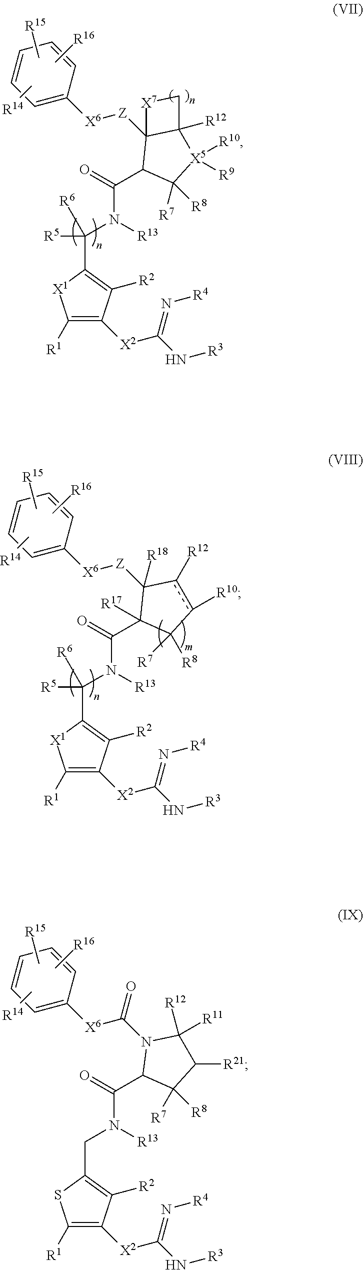

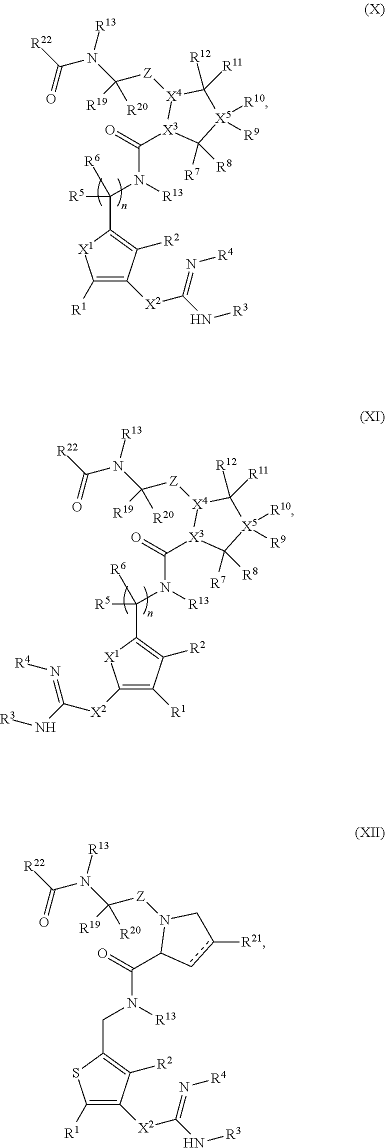

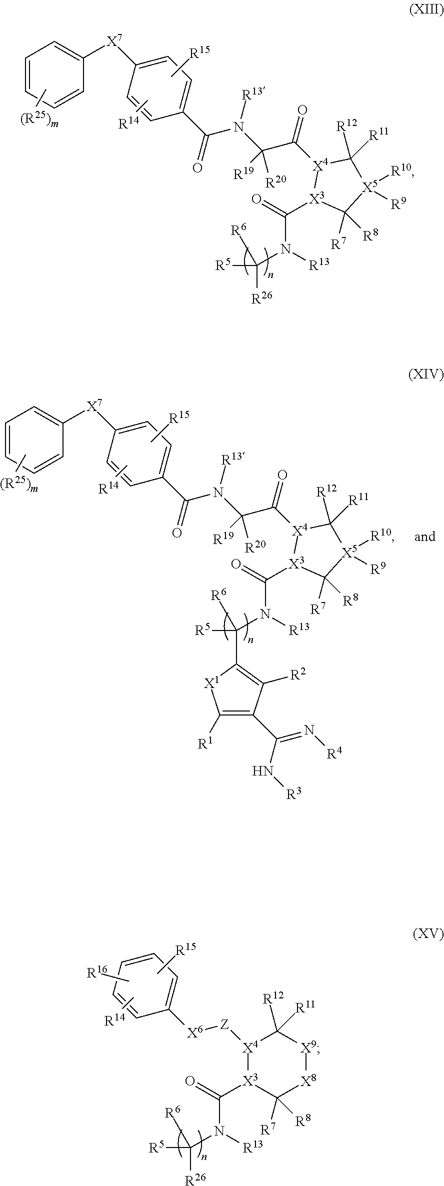

or a pharmaceutically acceptable salt, prodrug, or isolated isomer thereof;
wherein:
each n is independently 0, 1, 2, or 3;
each m is independently 0, 1, 2, or 3;
o is 0, 1, or 2;

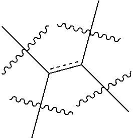

is either a single or a double bond;
Z is CH₂, C(CH₂), or C(O);
X¹ is selected from S, O, and N(R³⁰);
X² is selected from bond, N(R³⁰), and —O—N(R³⁰)—;
X³ is selected from N and C(R¹⁷);
X⁴ is selected from N and C(R¹³);
wherein only one of X³ and X⁴ can be N;
X⁵ is C, Si, or S;
X⁶ is selected from

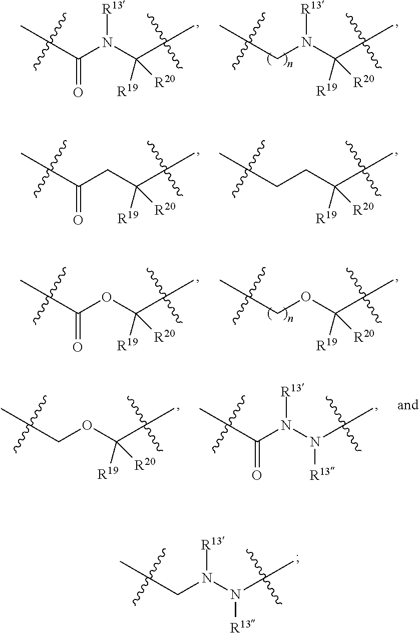

X⁷ is selected from O, S, N(R³⁰), and CR^{5′}R^{6′};
each X⁸ and X⁹ is independently selected from O, S, NR³⁰, CR⁹R¹⁰, CR⁵R⁶. and CH₂; wherein X⁸ and X⁹ cannot both be the same group
X¹¹ is selected from N and CR¹;
X¹² is selected from N and CR²
R¹ and R² are independently selected from hydrogen, halogen, C₁-C₆alkyl, C₂-C₆alkenyl, C₂-C₆alkynyl, C₁-C₆haloalkyl, —OR³⁰, —SR³⁰, —N(R³⁰)₂, —C(O)R³¹, —S(O)R³¹, —S(O)₂R³¹, heterocycle, aryl, heteroaryl, cyano, and nitro; each of which R¹ and R² groups other than hydrogen, halogen, cyano, and nitro are optionally substituted with 1, 2, 3, or 4 substituents independently selected from C₁-C₆alkyl, C₂-C₆alkenyl, C₂-C₆alkynyl, halogen, C₁-C₆haloalkyl, —OR³⁰, —SR³⁰, —N(R³⁰)₂, —C(O)R³¹, —S(O)R³¹, —S(O)₂R³¹, heterocycle, aryl, heteroaryl, cyano, and nitro;
R³ and R⁴ are independently selected from hydrogen, nitro, —S(O)₂R³¹, C(O)R³¹, —SR³⁰, and —OR³⁰;
or R³ and R⁴ are instead combined to form a dihydrooxadiazole optionally substituted with 1, 2, or 3 substituents independently selected from C₁-C₆alkyl, C₁-C₆haloalkyl, —OR³⁰, and oxo; an oxadiazole optionally substituted with 1, 2, or 3 substituents independently selected from C₁-C₆alkyl, C₁-C₆haloalkyl, and —OR³⁰; an imidazole optionally substituted with 1, 2, or 3 substituents independently selected from C₁-C₆alkyl, C₁-C₆haloalkyl, and —OR³⁰; or a dihydroimidazole optionally substituted 1, 2, or 3 substituents independently selected from C₁-C₆alkyl, C₁-C₆haloalkyl, —OR³⁰, and oxo;
R⁵, R^{5′}, R⁶, and R^{6′} are each independently selected from hydrogen, halogen, C₁-C₆alkyl, C₁-C₆haloalkyl, C₁-C₆hydroxyalkyl, —OR³⁰, —N(R³⁰)₂, and C(O)R³¹, wherein, when on carbons adjacent to each other, a R⁵ and a R⁶ group may optionally be replaced by a carbon-carbon double bond;
or, when n is 1, R⁵ and R⁶, together with the carbon to which they are attached, are replaced with —SO₂—;
or R⁵ and R⁶, together with the carbon atom to which they are attached, combine to form cyclopropyl;
R⁷, R⁸, R⁹, R¹⁰, R¹¹, and R¹² are independently selected from hydrogen, halogen, C₁-C₆alkyl, C₂-C₆alkenyl, C₂-C₆alkynyl, C₁-C₆haloalkyl, —OR³⁰, —SR³⁰, —N(R³⁰)₂, —C(O)R³¹, —S(O)R³¹, —S(O)₂R³¹, —S(O)(NR³¹)R³¹, carbocycle, heterocycle, aryl, and heteroaryl, each of which R⁷, R⁸, R⁹, R¹⁰, R¹¹, and R¹² groups other than hydrogen and halogen are optionally substituted with 1, 2, 3, or 4 substituents independently selected from C₁-C₆alkyl, C₂-C₆alkenyl, C₂-C₆alkynyl, halogen, C₁-C₆haloalkyl, —OR³⁰, —SR³⁰, —N(R³⁰)₂, —C(O)R³¹, —S(O)R³¹, —S(O)₂R³¹, heterocycle, aryl, heteroaryl, cyano, nitro, and azido;
or R⁷ and R⁸ may be taken together with the carbon to which they are attached to form a 3- to 6-membered carbocyclic spiro ring or a 4- to 6-membered heterocyclic spiro ring containing 1 or 2 heteroatoms independently chosen from N, O, and S, wherein the carbocyclic spiro ring and heterocyclic spiro ring are optionally substituted with one or more halogen, C₁-C₆alkyl, C₁-C₆ haloalkyl, —OR³⁰, —SR³⁰, or —N(R³⁰)₂;
or R⁷ and R⁸ may be taken together with the carbon to which they are attached to form

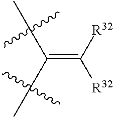

or carbonyl;
or R⁹ and R¹⁰ may be taken together with the atom to which they are attached to form a 3- to 6-membered carbocyclic spiro ring or a 4- to 6-membered heterocyclic spiro ring containing 1 or 2 heteroatoms independently chosen from N, O, and S, wherein the carbocyclic spiro ring and heterocyclic spiro ring are optionally substituted with one or more halogen, C₁-C₆alkyl, C₁-C₆ haloalkyl, —OR³⁰, —SR³⁰, or —N(R³⁰)₂;
or R⁹ and R¹⁰ may be taken together with the atom to which they are attached to form

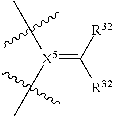

or carbonyl;
or R¹¹ and R¹² may be taken together with the carbon to which they are attached to form a 3- to 6-membered carbocyclic spiro ring or a 4- to 6-membered heterocyclic spiro ring containing 1 or 2 heteroatoms independently chosen from N, O, and S, wherein the carbocyclic spiro ring and heterocyclic spiro ring are optionally substituted with one or more halogen, C₁-C₆alkyl, C₁-C₆ haloalkyl, —OR³⁰, —SR³⁰, or —N(R³⁰)₂;
or R¹¹ and R¹² may be taken together with the carbon to which they are attached to form

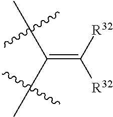

or carbonyl;
or R⁷ and R⁹ are taken together with the atoms to which they are attached to form a 4- to 8-membered carbocycle or a 4- to 8-membered heterocycle containing 1 or 2 heteroatoms independently chosen from N, O, and S;
or R⁹ and R¹¹ are taken together with the atoms to which they are attached to form a 4- to 8-membered carbocycle or a 4- to 8-membered heterocycle containing 1 or 2 heteroatoms independently chosen from N, O, and S;
or R⁷ and R¹¹ are taken together with the atoms to which they are attached to form a 1 or 2 carbon bridge;
or, when X⁵ is S, R⁹ and R¹⁰ are absent;
each R¹³ is independently selected from hydrogen, C₁-C₆alkyl, and OH;
or R¹³ and R²⁶, together with the atoms to which they are attached, form a heterocycle optionally substituted with R²⁷;
or R¹³, together with the nitrogen atom to which it is attached, is replaced with —O—;
each R^{13′} and R^{13″} is independently selected from hydrogen and C₁-C₆alkyl;
or R^{13′} and R¹⁴, together with the atoms to which they are attached, combine to form a 5- or 6-membered heterocycle containing one N;
R¹⁴, R¹⁵, and R¹⁶ are independently selected from hydrogen, halogen, C₁-C₆alkyl, C₂-C₆alkenyl, C₂-C₆alkynyl, C₁-C₆haloalkyl, —C₁-C₆alkyl-aryl. —OR³⁰, —SR³⁰, —N(R³⁰)₂, —C(O)R³¹, —S(O)R³¹, —S(O)₂R³¹, heterocycle, aryl, heteroaryl, cyano, and nitro each of which R¹⁴, R¹⁵, and R¹⁶ groups other than hydrogen, halogen, cyano, and nitro are optionally substituted with 1, 2, 3, or 4 substituents independently selected from C₁-C₆alkyl, C₂-C₆alkenyl, C₂-C₆alkynyl, halogen, C₁-C₆haloalkyl, —OR³⁰, —SR³⁰, —N(R³⁰)₂, —C(O)R³¹, —S(O)R³¹, —S(O)₂R³¹, heterocycle, aryl, heteroaryl, cyano, and nitro;
R¹⁷ and R¹⁸ are independently selected from hydrogen, halogen, C₁-C₆alkyl, C₁-C₆haloalkyl, —OR³⁰, and —N(R³⁰)₂;
or R¹⁷ and R¹⁸ are taken together with the carbons to which they are attached to form a double bond;
R¹⁹ and R²⁰ are independently selected from hydrogen, C₁-C₆alkyl, C₅-C₁₀ bicyclic carbocycle, C₄-C₆heterocycle, halogen, C₁-C₆haloalkyl, —OR³⁰, —N(R³⁰)₂, —(CH₂)ₙ—R³³, and

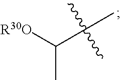

R²¹ is selected from C₁-C₆alkyl and —O—C₁-C₆alkyl;
R²² is selected from —C₁-C₆alkyl-R²³, —C₂-C₆alkenyl-R²³, —C₂-C₆alkynyl-R²³, -heteroaryl-R²³, -fluorenyl-R²³, and bicyclic cycloalkyl-R²³, each of which R²² is optionally substituted with 1, 2, 3, or 4 substituents independently selected from C₁-C₆alkyl, C₂-C₆alkenyl, C₂-C₆alkynyl, halogen, C₁-C₆haloalkyl, —OR³⁰, —SR³⁰, —N(R³⁰)₂, —C(O)R³¹, —S(O)R³¹, —S(O)₂R³¹, heterocycle, aryl, heteroaryl, cyano, and nitro;
R²³ is selected from hydrogen, sugar, —OR³⁰, —SR³⁰, —N(R³⁰)₂, —C(O)R³¹, —S(O)R³¹, and —S(O)₂R³¹;
each R²⁵ is independently selected from hydrogen, halogen, C₁-C₆alkyl, C₂-C₆alkenyl, C₂-C₆alkynyl, C₁-C₆haloalkyl, —OR³⁰, —SR³⁰, —N(R³⁰)₂, —C(O)R³¹, —S(O)R³¹, —S(O)₂R³¹, —S(O)(NR³¹)R³¹, —P(O)(OR³¹)R³¹, heterocycle, aryl, heteroaryl, cyano, and nitro; each of which R¹ and R² groups other than hydrogen, halogen, cyano, and nitro are optionally substituted with 1, 2, 3, or 4 substituents independently selected from C₁-C₆alkyl, C₂-C₆alkenyl, C₂-C₆alkynyl, halogen, C₁-C₆haloalkyl, —OR³⁰, —SR³⁰, —N(R³⁰)₂, —C(O)R³¹, —S(O)R³¹, —S(O)₂R³¹, heterocycle, aryl, heteroaryl, cyano, and nitro;
R²⁶ is selected from

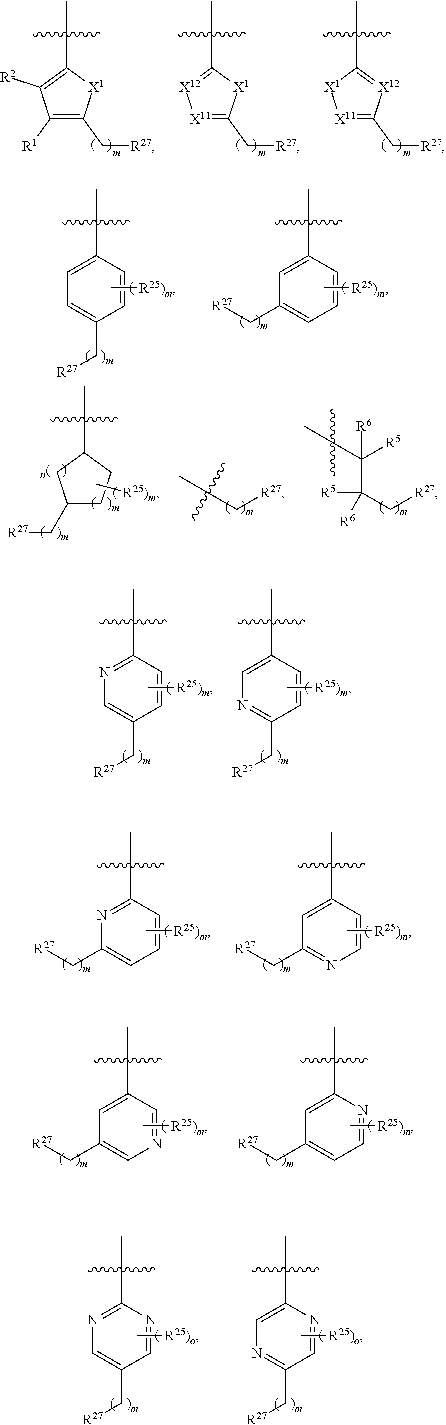

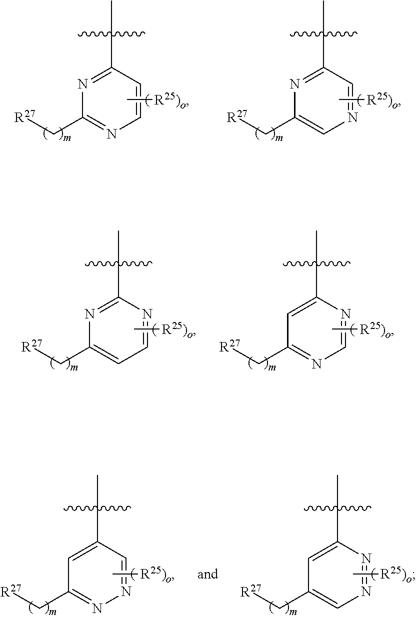

R²⁷ is selected from

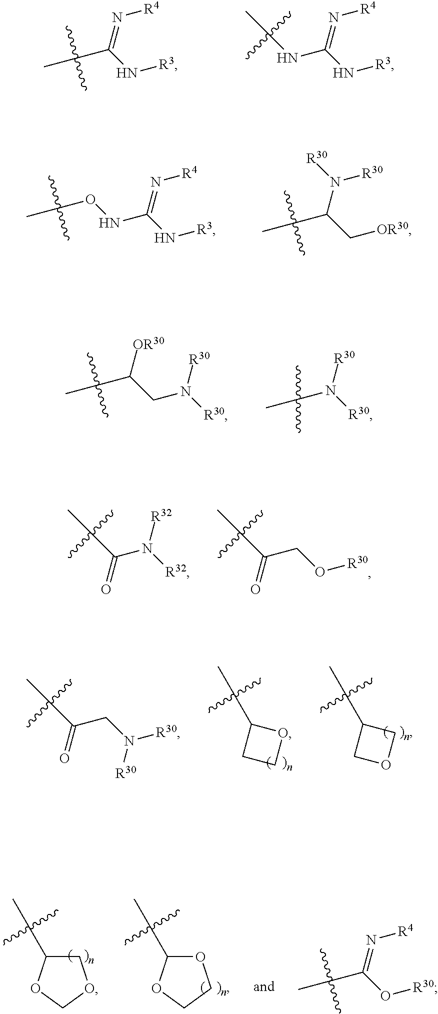

each R³⁰ is independently selected from hydrogen, C₁-C₆alkyl, C₁-C₆haloalkyl, carbxocycle, aryl, heteroaryl, heterocycle, and C(O)R³¹, each R³⁰ other than C(O)R³¹ is optionally substituted with 1, 2, 3, or 4 substituents selected from C₁-C₆alkyl, halogen, SF₅, —C(O)R³¹, —N(R³⁰)₂, aryl, —OR³², —S(O)(NR³¹)R³¹, and carbocycle;
or R³⁰ and R⁴ in

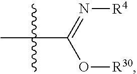

together with the N and O atoms to which each is attached and the carbon atom to which the N and O atoms are attached, combine to form oxadiazole optionally substituted with 1, 2, or 3 substituents independently selected from C₁-C₆alkyl, C₁-C₆haloalkyl, and —OR³⁰;
each R³¹ is independently selected from hydrogen, C₁-C₆alkyl, C₁-C₆haloalkyl, —OR³², —SR³², —N(R³²)₂, heterocycle, aryl, and heteroaryl;
each R³² is independently selected from hydrogen, halogen, C₁-C₆alkyl, and C₁-C₆haloalkyl; and
each R³³ is independently selected from hydrogen, guanidine, heteroaryl, aryl, —C₆HS—OR³⁰; —OR³⁰, —SR³⁰, —SeR³⁰, —N(R³⁰)₂, —C(O)R³¹.

8. The compound of embodiment 7, wherein for compounds of Formula I and Formula II at least one of the following is satisfied:
a. X³ is C(R¹⁷) and X⁴ is C(R¹⁸);
b. R¹⁷ is selected from halogen, C₁-C₆alkyl, C₁-C₆haloalkyl, —OR³⁰, and —N(R³⁰)₂;
c. X⁵ is Si;
d. X⁵ is S and at least two of R⁷, R⁸, R¹¹, and R¹² are not hydrogen, no more than one of R⁷ and R⁸ is halogen, and no more than one of R¹¹ and R¹² is halogen;
e. Z is C(CH₂);
f. Z is CH₂;
g. R⁷ and R⁸ are taken together with the carbon to which they are attached to form a 3- to 6-membered carbocyclic spiro ring; a 4- to 6-membered heterocyclic spiro ring containing 1 or 2 heteroatoms independently chosen from N, O, and S; R

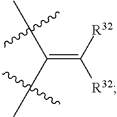

or a carbonyl;
h. R⁹ and R¹⁰ are taken together with the carbon to which they are attached to form a 3- to 6-membered carbocyclic spiro ring; a 4- to 6-membered heterocyclic spiro ring containing 1 or 2 heteroatoms independently chosen from N, O, and S;

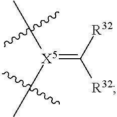

or a carbonyl;
i. R¹¹ and R¹² are taken together with the carbon to which they are attached to form a 3- to 6-membered carbocyclic spiro ring; a 4- to 6-membered heterocyclic spiro ring containing 1 or 2 heteroatoms independently chosen from N, O, and S;

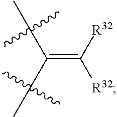

or a carbonyl;
j. R⁷ and R⁹ are taken together with the atoms to which they are attached to form a 4- to 8-membered carbocycle or a 4- to 8-membered heterocycle containing 1 or 2 heteroatoms independently chosen from N, O, and S; and R¹⁰ or R¹² is not hydrogen;
k. R⁹ and R¹¹ are taken together with the atoms to which they are attached to form a 4- to 8-membered carbocycle or a 4- to 8-membered heterocycle containing 1 or 2 heteroatoms independently chosen from N, O, and S; and R⁸ or R¹⁰ is not hydrogen;
l. R⁷ and R¹¹ are taken together with the atoms to which they are attached to form a 1 or 2 carbon bridge;
m. X⁶ is selected from

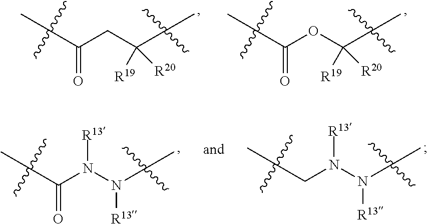

n. at least one of R³ and R⁴ is CN, —SR³⁰ nitro, —S(O)₂R³¹, or C(O)R³¹;
o. R³ and R⁴ are combined to form a dihydroxadiazole optionally substituted with 1, 2, or 3 substituents independently selected from C₁-C₆alkyl, C₁-C₆haloalkyl, —OR³⁰, and oxo; an oxadiazole optionally substituted with 1, 2, or 3 substituents independently selected from C₁-C₆alkyl, C₁-C₆haloalkyl, and —OR³⁰; an imidazole optionally substituted with 1, 2, or 3 substituents independently selected from C₁-C₆alkyl, C₁-C₆haloalkyl, and —OR³⁰; or a dihydroimidazole optionally substituted 1, 2, or 3 substituents independently selected from C₁-C₆alkyl, C₁-C₆haloalkyl, —OR³⁰, and oxo;
p. R²⁷ is

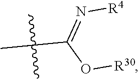

and R³⁰ and R⁴ in

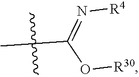

together with the N and O atoms to which each is attached and the carbon atom to which the N and O atoms are attached, combine to form oxadiazole optionally substituted with 1, 2, or 3 substituents independently selected from C₁-C₆alkyl, C₁-C₆haloalkyl, and —OR³⁰;
   wherein for compounds of Formula X and Formula XI at least one of the following is satisfied:
a. X³ is C(R¹⁷) and X⁴ is C(R¹⁸);
b. R¹⁷ is selected from halogen, C₁-C₆alkyl, C₁-C₆haloalkyl, —OR³⁰, and —N(R³⁰)₂;
c. X⁵ is Si;
d. X⁵ is S and at least two of R⁷, R⁸, R¹¹, and R¹² are not hydrogen, no more than one of R⁷ and R⁸ is halogen, and no more than one of R¹¹ and R¹² is halogen;
e. Z is C(CH₂);
f. Z is CH₂;
g. R⁷ and R⁸ are taken together with the carbon to which they are attached to form a 3- to 6-membered carbocyclic spiro ring; a 4- to 6-membered heterocyclic spiro ring containing 1 or 2 heteroatoms independently chosen from N, O, and S;

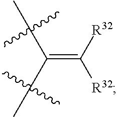

or a carbonyl;
h. R⁹ and R¹⁰ are taken together with the carbon to which they are attached to form a 3- to 6-membered carbocyclic spiro ring; a 4- to 6-membered heterocyclic spiro ring containing 1 or 2 heteroatoms independently chosen from N, O, and S;

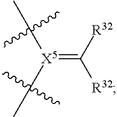

or a carbonyl;
i. R⁹ and R¹¹ are taken together with the atoms to which they are attached to form a 4- to 8-membered carbocycle or a 4- to 8-membered heterocycle containing 1 or 2 heteroatoms independently chosen from N, O, and S; and R¹⁰ is not hydrogen;
j. R¹¹ and R¹² are taken together with the carbon to which they are attached to form a 3- to 6-membered carbocyclic spiro ring; a 4- to 6-membered heterocyclic spiro ring containing 1 or 2 heteroatoms independently chosen from N, O, and S;

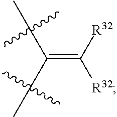

or a carbonyl;
k. R⁷ and R⁹ are taken together with the atoms to which they are attached to form a 4- to 8-membered carbocycle or a 4- to 8-membered heterocycle containing 1 or 2 heteroatoms independently chosen from N, O, and S; and R¹⁰ is not hydrogen;
l. R⁷ and R¹¹ are taken together with the atoms to which they are attached to form a 1 or 2 carbon bridge;
m. R²² is substituted with at least three OR³⁰ groups;
n. R²³ is a sugar;
o. at least one of R³ and R⁴ is —SR³⁰ or C(O)R³¹;
p. R³ and R⁴ are combined to form a dihydroxadiazole optionally substituted with 1, 2, or 3 substituents independently selected from C₁-C₆alkyl, C₁-C₆haloalkyl, —OR³⁰, and oxo; an oxadiazole optionally substituted with 1, 2, or 3 substituents independently selected from C₁-C₆alkyl, C₁-C₆haloalkyl, and —OR³⁰; an imidazole optionally substituted with 1, 2, or 3 substituents independently selected from C₁-C₆alkyl, C₁-C₆haloalkyl, and —OR³⁰; or a dihydroimidazole optionally substituted 1, 2, or 3 substituents independently selected from C₁-C₆alkyl, C₁-C₆haloalkyl, —OR³⁰, and oxo; or
q. R²⁷ is

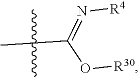

and R³⁰ and R⁴ in

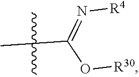

together with the N and O atoms to which each is attached and the carbon atom to which the N and O atoms are attached, combine to form oxadiazole optionally substituted with 1, 2, or 3 substituents independently selected from C₁-C₆alkyl, C₁-C₆haloalkyl, and —OR³⁰;
   wherein for compounds of Formula XIV at least one of the following is satisfied:
a. X¹ is O or N(R³⁰);
b. R¹⁴ is not hydrogen;
c. R¹ is not hydrogen;
d. R² is not hydrogen;
e. R³ is not hydrogen; or
f. R⁴ is not hydrogen.

9. The compound of any one of embodiments 1-8, wherein n is 0.

10. The compound of any one of embodiments 1-8, wherein n is 1.

11. The compound of any one of embodiments 1-8, where n is 2.

12. The compound of any one of embodiments 1-11, wherein R⁵ and R⁶, together with the carbon to which they are attached, is

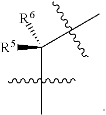

13. The compound of any one of embodiments 1-12, wherein R⁵ is methyl and R⁶ is H.

14. The compound of any one of embodiments 1-13, wherein each m is independently 0 or 1.

15. The compound of any one of embodiments 1-14, wherein Z is C(O).

16. The compound of any one of embodiments 1-15, wherein X¹ is S.

17. The compound of any one of embodiments 1-16, wherein X² is bond.

18. The compound of any one of embodiments 1-17, wherein X³ is C(R¹⁷).

19. The compound of any one of embodiments 1-18, wherein X⁴ is N.

20. The compound of any one of embodiments 1-19, wherein X⁵ is C.

21. The compound of any one of embodiments 1-19, wherein X⁵ is Si.

22. The compound of any one of embodiments 1-19, wherein X⁵ is S, and R⁹ and R¹⁰ are absent.

23. The compound of any one of embodiments 1-22, wherein X⁶ is

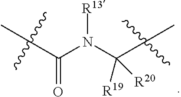

24. The compound of any one of embodiments 1-23, wherein X⁷ is O.

25. The compound of any one of embodiments 1-23, wherein X⁷ is CR^{5′}R^{6′}.

26. The compound of any one of embodiments 1-23, wherein X⁷ is S.

27. The compound of any one of embodiments 1-23, wherein X⁷ is N(R³⁰).

28. The compound of any one of embodiments 1-27, wherein X⁸ is CH.

29. The compound of any one of embodiments 1-27, wherein X⁸ is CH and X⁹ is N.

30. The compound of any one of embodiments 1-29, wherein X¹¹ and X¹² are both CH.

31. The compound of any one of embodiments 1-29, wherein one of X¹¹ and X¹² is CH and the other is N.

32. The compound of any one of embodiments 1-31, wherein R¹ and R² are independently selected from hydrogen, halogen, —OR³⁰, —SR³⁰, —N(R³⁰)₂, and C₁-C₆alkyl.

33. The compound of any one of embodiments 1-31, wherein R¹ and R² are independently selected from hydrogen, halogen, and C₁-C₆alkyl.

34. The compound of any one of embodiments 1-31, wherein R¹ and R² both hydrogen.

35. The compound of any one of embodiments 1-34, wherein R³ and R⁴ both hydrogen.

36. The compound of any one of embodiments 1-34, wherein R³ is hydrogen and R⁴ is hydroxyl.

37. The compound of any one of embodiments 1-34, wherein R³ is hydrogen and R⁴ is nitro.

38. The compound of any one of embodiments 1-34, wherein R³ is hydrogen and R⁴ is S(O)₂CH₃.

39. The compound of any one of embodiments 1-34, wherein R³ and R⁴ are combined to form a dihydroxadizol optionally substituted with 1, 2, or 3 substituents independently selected from C₁-C₆alkyl, C₁-C₆haloalkyl, —OR³⁰, and oxo.

40. The compound of any one of embodiments 1-34, wherein R³ and R⁴ are combined to form an oxadiazole optionally substituted with 1, 2, or 3 substituents independently selected from C₁-C₆alkyl, C₁-C₆haloalkyl, and —OR³⁰.

41. The compound of any one of embodiments 1-34, wherein R³ and R⁴ are combined to form an imidazole optionally substituted with 1, 2, or 3 substituents independently selected from C₁-C₆alkyl, C₁-C₆haloalkyl, and —OR³⁰.

42. The compound of any one of embodiments 1-34, wherein R³ and R⁴ are combined to form a dihydroimidazole optionally substituted 1, 2, or 3 substituents independently selected from C₁-C₆alkyl, C₁-C₆haloalkyl, —OR³⁰, and oxo.

43. The compound of any one of embodiments 1-42, wherein R⁵ and R⁶ are both hydrogen.

44. The compound of any one of embodiments 1-42, wherein n is 1, and R⁵ and R⁶, together with the carbon atom to which they are attached, are replaced with —SO₂—.

45. The compound of any one of embodiments 1-42, wherein R⁵ and R⁶, together with the carbon atom to which they are attached, combine to form cyclopropyl;

46. The compound of any one of embodiments 1-45, wherein R⁷ is hydrogen.

47. The compound of any one of embodiments 1-46, wherein R⁹ is hydrogen.

48. The compound of any one of embodiments 1-45, wherein R⁷ and R¹¹ are combined to form a 1-carbon bridge.

49. The compound of any one of embodiments 1-45, wherein R⁷ and R¹¹ are combined to form a 2-carbon bridge.

50. The compound of any one of embodiments 1-46, wherein R¹¹ is hydrogen.

51. The compound of any one of embodiments 1-46, wherein R⁹ and R¹¹ are combined to form a 4-8 membered carbocycle ring.

52. The compound of any one of embodiments 1-45, wherein R⁹ and R¹¹ are combined to form a cyclopropyl ring.

53. The compound of any one of embodiments 1-45, wherein R⁹ and R¹¹, together with the atoms to which they are attached, combine to form

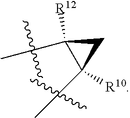

54. The compound of any one of embodiments 1-45, wherein R⁹ and R¹⁰ are taken together with the carbon to which they are attached to form

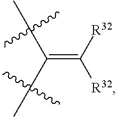

where R³² is fluoro.

55. The compound of any one of embodiments 1-53, wherein R¹⁰ is hydrogen.

56. The compound of any one of embodiments 1-53, wherein R¹⁰ is selected from halogen, C₁-C₆alkyl, C₂-C₆alkenyl, C₂-C₆alkynyl, C₁-C₆haloalkyl, —OR³⁰, —SR³⁰, —N(R³⁰)₂, —C(O)R³¹, —S(O)R³¹, and —S(O)₂R³¹, each R¹⁰ other than hydrogen and halogen are optionally substituted with 1, 2, 3, or 4 substituents independently selected from C₁-C₆alkyl, C₂-C₆alkenyl, C₂-C₆alkynyl, halogen, C₁-C₆haloalkyl, —OR³⁰, —SR³⁰, —N(R³⁰)₂, —C(O)R³¹, —S(O)R³¹, —S(O)₂R³¹, heterocycle, aryl, heteroaryl, cyano, nitro, and azido.

57. The compound of any one of embodiments 1-53, wherein R¹⁰ is selected from carbocycle, aryl, and heteroaryl, each of which is optionally substituted with 1, 2, 3, or 4 substituents independently selected from C₁-C₆alkyl, C₂-C₆alkenyl, C₂-C₆alkynyl, halogen, C₁-C₆haloalkyl, —OR³⁰, —SR³⁰, —N(R³⁰)₂, —C(O)R³¹, —S(O)R³¹, —S(O)₂R³¹, heterocycle, aryl, heteroaryl, cyano, nitro, and azido.

58. The compound of any one of embodiments 1-53, wherein R¹⁰ is methyl.

59. The compound of any one of embodiments 1-53, wherein R¹⁰ is azidomethyl.

60. The compound of any one of embodiments 1-53, wherein R¹⁰ is —OR³⁰.

61. The compound of any one of embodiments 1-53, wherein R¹⁰ is —OCHF₂.

62. The compound of any one of embodiments 1-53, wherein R¹⁰ is S-methylsulfonimidoyl.

63. The compound of any one of embodiments 1-53, wherein R¹⁰ is cycloalkyl.

64. The compound of any one of embodiments 1-53, wherein R¹⁰ is cyclopentyl.

65. The compound of any one of embodiments 1-53, wherein R¹⁰ is cyclohexyl.

66. The compound of any one of embodiments 1-45, wherein R⁹ and R¹⁰, together with the carbon atom to which they are attached, is

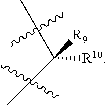

67. The compound of any one of embodiments 1-45, wherein R⁹ and R¹⁰ are combined to form a spirocycle.

68. The compound of any one of embodiments 1-45, wherein R⁹ and R¹⁰ are combined to form a 5-membered heterocycle spirocycle.

69. The compound of any one of embodiments 1-45, wherein R⁹ and R¹⁰ are combined to form a 5-membered carbocycle spirocycle.

70. The compound of any one of embodiments 1-45, wherein R⁹ and R¹⁰ are taken together with the carbon to which they are attached to form cyclopropyl optionally substituted with one or more halogen.

71. The compound of any one of embodiments 1-45, wherein R⁹ and R¹⁰ are taken together with the carbon to which they are attached to form

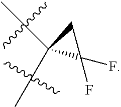

72. The compound of any one of embodiments 1-45, wherein R⁹ and R¹⁰ are taken together with the carbon to which they are attached to form

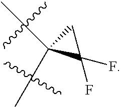

73. The compound of any one of embodiments 1-72, wherein R¹² is hydrogen.

74. The compound of any one of embodiments 1-73, wherein R⁸ is hydrogen.

75. The compound of any one of embodiments 1-74, wherein R¹³ is hydrogen.

76. The compound of any one of embodiments 1-74, wherein R¹³ is C₁-C₆alkyl.

77. The compound of any one of embodiments 1-74, where R¹³ is OH.

78. The compound of any one of embodiments 1-74, wherein R¹³ and R²⁶, together with the atoms to which they are attached, form a heterocycle optionally substituted with R²⁷.

79. The compound of any one of embodiments 1-74, R¹³ and R²⁶, together with the atoms to which they are attached, form

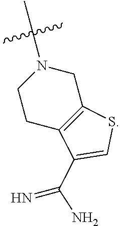 [SMILES: *N1CCc2c(C(=N)N)csc2C1]

80. The compound of any one of embodiments 1-74, wherein R¹³, together with the nitrogen atom to which it is attached, is replaced with —O—.

81. The compound of any one of embodiments 1-80, wherein R^{13′} is hydrogen.

82. The compound of any one of embodiments 1-80, wherein R^{13′} is C₁-C₆alkyl.

83. The compound of any one of embodiments 1-80, wherein R^{13′} and R¹⁴, together with the atoms to which they are attached, combine to form a 5- or 6-membered heterocycle containing one N.

84. The compound of any one of embodiments 1-83, wherein R^{13″} is hydrogen.

85. The compound of any one of embodiments 1-83, wherein R^{13″} is C₁-C₆alkyl.

86. The compound of any one of embodiments 1-85, wherein R¹⁴ is C₁-C₆alkyl.

87. The compound of any one of embodiments 1-85, wherein R¹⁴ is hydrogen.

88. The compound of any one of embodiments 1-85, wherein R¹⁴ is halogen.

89. The compound of any one of embodiments 1-85, wherein R¹⁴ is haloalkyl.

90. The compound of any one of embodiments 1-85, wherein R¹⁴ is OR³⁰.

91. The compound of any one of embodiments 1-85, wherein R¹⁴ is —O-phenyl.

92. The compound of any one of embodiments 1-91, wherein R¹⁵ is C₁-C₆alkyl.

93. The compound of any one of embodiments 1-91, wherein R¹⁵ is hydrogen.

94. The compound of any one of embodiments 1-91, wherein R¹⁵ is halogen.

95. The compound of any one of embodiments 1-91, wherein R¹⁵ is haloalkyl.

96. The compound of any one of embodiments 1-91, wherein R¹⁵ is OR³⁰.

97. The compound of any one of embodiments 1-91, wherein R¹⁵ is —O-phenyl.

98. The compound of any one of embodiments 1-97, wherein R¹⁶ is C₁-C₆alkyl.

99. The compound of any one of embodiments 1-97, wherein R¹⁶ is hydrogen.

100. The compound of any one of embodiments 1-97, wherein R¹⁶ is halogen.

101. The compound of any one of embodiments 1-97, wherein R¹⁶ is haloalkyl.

102. The compound of any one of embodiments 1-97, wherein R¹⁶ is OR³⁰.

103. The compound of any one of embodiments 1-97, wherein R¹⁶ is —O-phenyl.

104. The compound of any one of embodiments 1-103, wherein R¹⁷ is hydrogen.

105. The compound of any one of embodiments 1-104, wherein R¹⁸ is hydrogen.

106. The compound of any one of embodiments 1-105, wherein R¹⁹ is hydrogen.

107. The compound of any one of embodiments 1-105, wherein R¹⁹ is selected from C₁-C₆alkyl, C₅-C₁₀ bicyclic carbocycle, C₄-C₆heterocycle, halogen, C₁-C₆haloalkyl, —OR³⁰, —N(R³⁰)₂, —(CH₂)ₙ—R³³, and

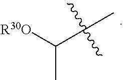

108. The compound of any one of embodiments 1-107, wherein R²⁰ is hydrogen.

109. The compound of any one of embodiments 1-107, wherein R²⁰ is selected from C₁-C₆alkyl, C₅-C₁₀ bicyclic carbocycle, C₄-C₆heterocycle, halogen, C₁-C₆haloalkyl, —OR³⁰, —N(R³⁰)₂, —(CH₂)ₙ—R³³, and

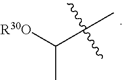

110. The compound of any one of embodiments 1-107, wherein R²⁰ is —(CH₂)ₙ—R³³.

111. The compound of any one of embodiments 1-110, wherein R²¹ is C₁-C₆haloalkyl.

112. The compound of any one of embodiments 1-110, wherein R²¹ is —O—C₁-C₆haloalkyl.

113. The compound of any one of embodiments 1-110, wherein R²¹ is phenyl, optionally substituted with 1, 2, 3, or 4 substituents independently selected from SF₅, C₁-C₆alkyl, C₂-C₆alkenyl, C₂-C₆alkynyl, halogen, C₁-C₆haloalkyl, —OR³⁰, —SR³⁰, —N(R³⁰)₂, —C(O)R³¹, —S(O)R³¹, —S(O)₂R³¹, heterocycle, aryl, heteroaryl, cyano, and nitro.

114. The compound of any one of embodiments 1-110, wherein R²¹ is heteroaryl, optionally substituted with 1, 2, 3, or 4 substituents independently selected from SF₅, C₁-C₆alkyl, C₂-C₆alkenyl, C₂-C₆alkynyl, halogen, C₁-C₆haloalkyl, —OR³⁰, —SR³⁰, —N(R³⁰)₂, —C(O)R³¹, —S(O)R³¹, —S(O)₂R³¹, heterocycle, aryl, heteroaryl, cyano, and nitro.

115. The compound of embodiment 113 or 114, wherein R²¹ is not substituted.

116. The compound of embodiment 113 or 114, wherein R²¹ is substituted with at least 1 halogen group.

117. The compound of embodiment 113 or 114, wherein R²¹ is substituted with at least 1 C₁-C₆alkyl group.

118. The compound of embodiment 113 or 114, wherein R²¹ is substituted with 1 fluoro group.

119. The compound of embodiment 113 or 114, wherein R²¹ is substituted with 1 methyl group.

120. The compound of any one of embodiments 111-119, wherein R²¹, together with the carbon to which it is attached, is

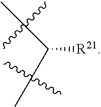

121. The compound of any one of embodiments 1-120, wherein R²² is —C₁-C₆alkyl-R²³ optionally substituted with 1, 2, 3, or 4 substituents independently selected from C₁-C₆alkyl, C₂-C₆alkenyl, C₂-C₆alkynyl, halogen, C₁-C₆haloalkyl, —OR³⁰, —SR³⁰, —N(R³⁰)₂, —C(O)R³¹, —S(O)R³¹, —S(O)₂R³¹, heterocycle, aryl, heteroaryl, cyano, and nitro.

122. The compound of any one of embodiments 1-120, wherein R²² is —C₃-C₆alkyl-R²³ optionally substituted with 1, 2, 3, or 4 substituents independently selected from C₁-C₆alkyl, C₂-C₆alkenyl, C₂-C₆alkynyl, halogen, C₁-C₆haloalkyl, —OR³⁰, —SR³⁰, —N(R³⁰)₂, —C(O)R³¹, —S(O)R³¹, —S(O)₂R³¹, heterocycle, aryl, heteroaryl, cyano, and nitro.

123. The compound of any one of embodiments 1-120, wherein R²² is bicyclic cycloalkyl-R²³ optionally substituted with 1, 2, 3, or 4 substituents independently selected from C₁-C₆alkyl, C₂-C₆alkenyl, C₂-C₆alkynyl, halogen, C₁-C₆haloalkyl, —OR³⁰, —SR³⁰, —N(R³⁰)₂, —C(O)R³¹, —S(O)R³¹, —S(O)₂R³¹, heterocycle, aryl, heteroaryl, cyano, and nitro.

124. The compound of any one of embodiments 1-120, wherein R²² is -heteroaryl-R²³ optionally substituted with 1, 2, 3, or 4 substituents independently selected from C₁-C₆alkyl, C₂-C₆alkenyl, C₂-C₆alkynyl, halogen, C₁-C₆haloalkyl, —OR³⁰, —SR³⁰, —N(R³⁰)₂, —C(O)R³¹, —S(O)R³¹, —S(O)₂R³¹, heterocycle, aryl, heteroaryl, cyano, and nitro.

125. The compound of any one of embodiments 1-120, wherein R²² is

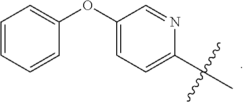 [SMILES: *c1ccc(Oc2ccccc2)cn1]

126. The compound of any one of embodiments 1-120, wherein R²² is

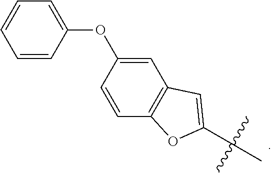 [SMILES: *c1cc2cc(Oc3ccccc3)ccc2o1]

127. The compound of any one of embodiments 1-120, wherein R²² is -carbocycle-R²³ optionally substituted with 1, 2, 3, or 4 substituents independently selected from C₁-C₆alkyl, C₂-C₆alkenyl, C₂-C₆alkynyl, halogen, C₁-C₆haloalkyl, —OR³⁰, —SR³⁰, —N(R³⁰)₂, —C(O)R³¹, —S(O)R³¹, —S(O)₂R³¹, heterocycle, aryl, heteroaryl, cyano, and nitro.

128. The compound of any one of embodiments 1-120, wherein R²² is

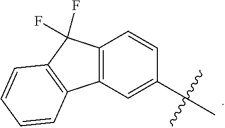 [SMILES: *c1ccc2c(c1)-c1ccccc1C2(F)F]

129. The compound of any one of embodiments 1-124 and 127, wherein R²² is unsubstituted.

130. The compound of any one of embodiments 1-129, wherein R²³ is hydrogen.

131. The compound of any one of embodiments 1-129, wherein R²³ is sugar.

132. The compound of any one of embodiments 1-129, wherein R²³ is —OR³⁰.

133. The compound of any one of embodiments 1-129, wherein R²³ is SR³⁰, —N(R³⁰)₂, —C(O)R³¹, —S(O)R³¹, or —S(O)₂R³¹.

134. The compound of any one of embodiments 1-133, wherein R²⁵ is C₁-C₆alkyl.

135. The compound of any one of embodiments 1-133, wherein R²⁵ is hydrogen.

136. The compound of any one of embodiments 1-133, wherein R²⁵ is halogen.

137. The compound of any one of embodiments 1-133, wherein R²⁵ is haloalkyl.

138. The compound of any one of embodiments 1-133, wherein R²⁵ is OR³⁰.

139. The compound of any one of embodiments 1-133, wherein R²⁵ is —O-phenyl.

140. The compound of any one of embodiments 1-133, wherein R²⁵ is SF₅.

141. The compound of any one of embodiments 1-133, wherein R²⁵ is S-methylsulfonimidoyl.

142. The compound of any one of embodiments 1-133, wherein R²⁵ is methylphosphinyl.

143. The compound of any one of embodiments 1-142, wherein R²⁶ is

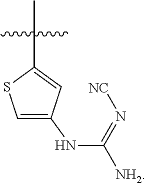 [SMILES: *c1cc(N/C(N)=N\C#N)cs1]

144. The compound of any one of embodiments 1-142, wherein R²⁶ is selected from:

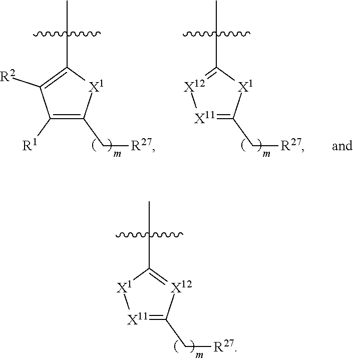

145. The compound of any one of embodiments 1-142, wherein R²⁶ is

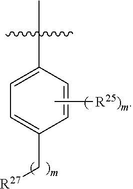

146. The compound of any one of embodiments 1-142, wherein R²⁶ is

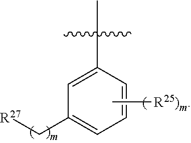

147. The compound of any one of embodiments 1-142, wherein R²⁶ is

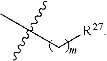

148. The compound of any one of embodiments 1-142, wherein R²⁶ is selected from:

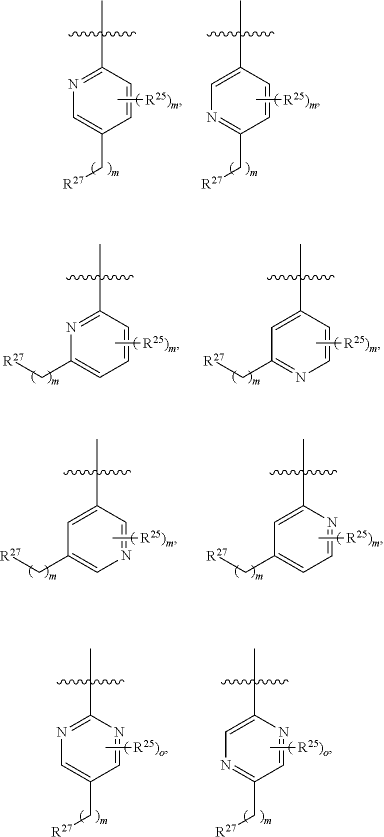

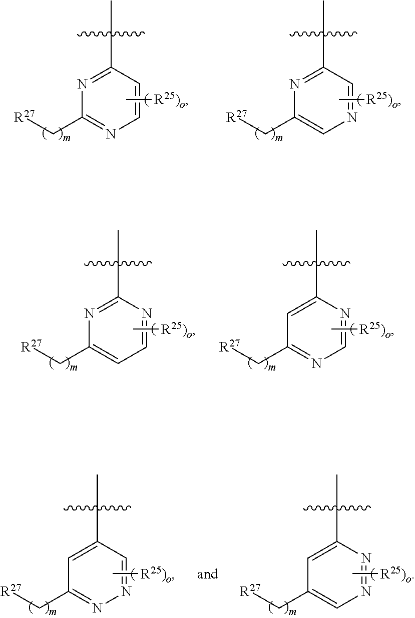

149. The compound of any one of embodiments 1-142, wherein R²⁶ is

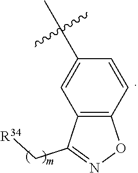

150. The compound of any one of embodiments 1-149, wherein R²⁷ is

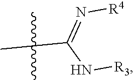

151. The compound of any one of embodiments 1-149, wherein R²⁷ is

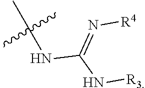

152. The compound of any one of embodiments 1-149, wherein R²⁷ is

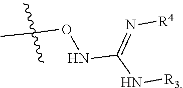

153. The compound of any one of embodiments 1-149, wherein R²⁷ is

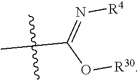

154. The compound of any one of embodiments 1-153, wherein R³⁰ is hydrogen.

155. The compound of any one of embodiments 1-153, wherein R³⁰ is C₁-C₆alkyl.

156. The compound of any one of embodiments 1-153, wherein R³⁰ is methyl.

157. The compound of any one of embodiments 1-153, wherein R³⁰ is C₁-C₆alkyl substituted with carbocycle.

158. The compound of any one of embodiments 1-153, wherein R³⁰ is methyl substituted with cycloalkyl.

159. The compound of any one of embodiments 1-153, wherein R³⁰ is methyl substituted with cyclohexyl.

160. The compound of any one of embodiments 1-153, wherein R³⁰ is methyl substituted with cyclopropyl.

161. The compound of any one of embodiments 1-153, wherein R³⁰ is C₁-C₆haloalkyl.

162. The compound of any one of embodiments 1-153, wherein R³⁰ is CF₃.

163. The compound of any one of embodiments 1-153, wherein R³⁰ is C(O)R³¹.

164. The compound of any one of embodiments 1-153, wherein R³⁰ is cycloalkyl.

165. The compound of any one of embodiments 1-153, wherein R³⁰ is cyclopropyl.

166. The compound of any one of embodiments 1-153, wherein R³⁰ is cyclohexyl.

167. The compound of any one of embodiments 1-153, wherein R³⁰ is aryl.

168. The compound of any one of embodiments 1-153, wherein R³⁰ is aryl substituted with C(O)R³¹.

169. The compound of any one of embodiments 1-153, wherein R³⁰ is 4-fluorophenyl.

170. The compound of any one of embodiments 1-153, wherein R³⁰ is 4-fluorophenyl.

171. The compound of any one of embodiments 1-153, wherein R³⁰ is 4-carboxyphenyl.

172. The compound of any one of embodiments 1-153, wherein R³⁰ is 4-ethoxycarbonylphenyl.

173. The compound of any one of embodiments 1-153, wherein R³⁰ is 4-(S-methylsulfonimidoyl)phenyl.

174. The compound of any one of embodiments 1-153, wherein R³⁰ is p-tolyl.

175. The compound of any one of claims 1-153, wherein R³⁰ is 4-(pentafluoro-λ⁶-sulfanyl)phenyl.

176. The compound of any one of embodiments 1-175, wherein R³¹ is hydrogen.

177. The compound of any one of embodiments 1-175, wherein R³¹ is C₁-C₆alkyl.

178. The compound of any one of embodiments 1-175, wherein R³¹ is methyl.

179. The compound of any one of embodiments 1-175, wherein R³¹ is C₁-C₆haloalkyl.

180. The compound of any one of embodiments 1-175, wherein R³¹ is CF₃.

181. The compound of any one of embodiments 1-175, wherein R³¹ is —OR³².

182. The compound of any one of embodiments 1-175, wherein R³¹ is —N(R³²)₂.

183. The compound of any one of embodiments 1-182, wherein R³² is hydrogen.

184. The compound of any one of embodiments 1-182, wherein R³² is C₁-C₆alkyl.

185. The compound of any one of embodiments 1-182, wherein R³² is halogen.

186. The compound of any one of embodiments 1-182, wherein R³² is fluoro.

187. The compound of any one of embodiments 1-186, wherein R³³ is hydrogen.

188. The compound of any one of embodiments 1-186, wherein R³³ is independently selected from heteroaryl, aryl, —C₆H₅—OR³⁰; —OR³⁰, —SR³⁰, —SeR³⁰, —N(R³⁰)₂, and —C(O)R³¹.

189. The compound of any one of embodiments 1-186, wherein R³³ is guanidine.

190. The compound of any one of embodiments 1-189, wherein the compound is of formula:

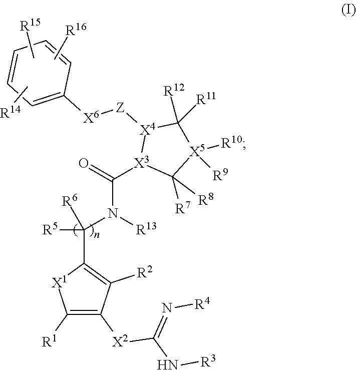

or a pharmaceutically acceptable salt thereof.

191. The compound of any one of embodiments 1-189, wherein the compound is of formula:

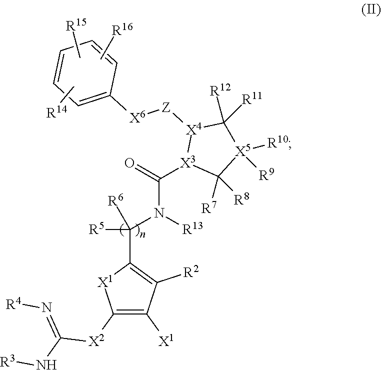

or a pharmaceutically acceptable salt thereof.

192. The compound of any one of embodiments 1-189, wherein the compound is of formula:

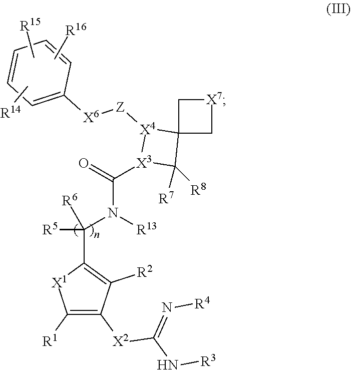

or a pharmaceutically acceptable salt thereof.

193. The compound of any one of embodiments 1-189, wherein the compound is of formula:

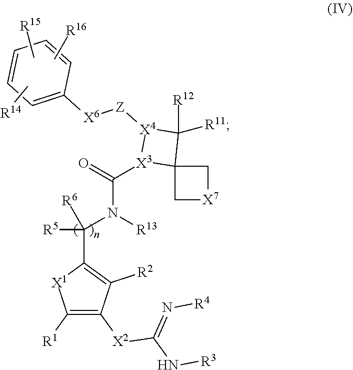

or a pharmaceutically acceptable salt thereof.

194. The compound of any one of embodiments 1-189, wherein the compound is of formula:

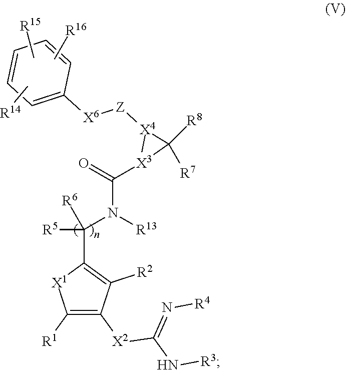

or a pharmaceutically acceptable salt thereof.

195. The compound of any one of embodiments 1-189, wherein the compound is of formula:

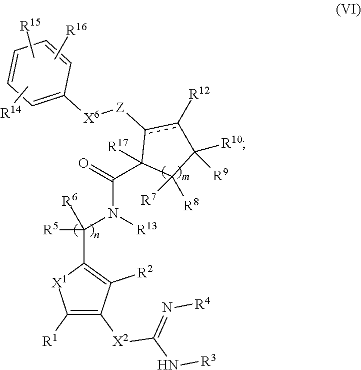

or a pharmaceutically acceptable salt thereof.

196. The compound of any one of embodiments 1-189, wherein the compound is of formula:

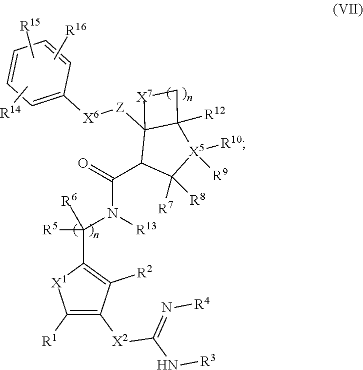

or a pharmaceutically acceptable salt thereof.

197. The compound of any one of embodiments 1-189, wherein the compound is of formula:

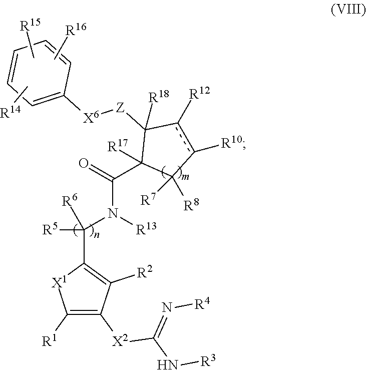

or a pharmaceutically acceptable salt thereof.

198. The compound of any one of embodiments 1-189, wherein the compound is of formula:

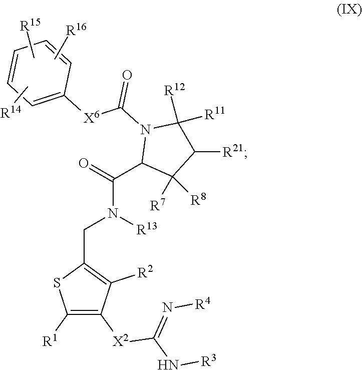

or a pharmaceutically acceptable salt thereof.

199. The compound of any one of embodiments 1-189, wherein the compound is of formula:

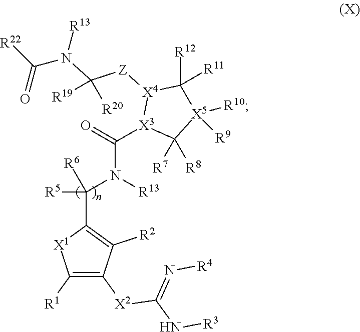

or a pharmaceutically acceptable salt thereof.

200. The compound of any one of embodiments 1-189, wherein the compound is of formula:

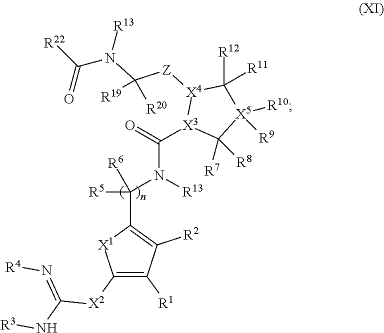

or a pharmaceutically acceptable salt thereof.

201. The compound of any one of embodiments 1-189, wherein the compound is of formula:

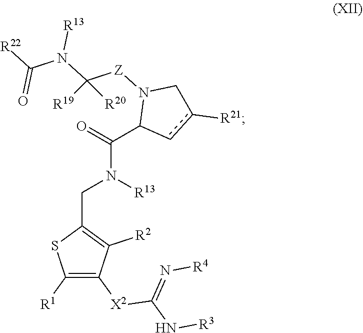

or a pharmaceutically acceptable salt thereof.

202. The compound of embodiment 201, wherein

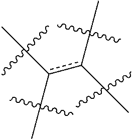

is a single bond.

203. The compound of embodiment 201, wherein

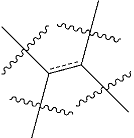

is a double bond.

204. The compound of any one of embodiments 1-189, wherein the compound is of formula:

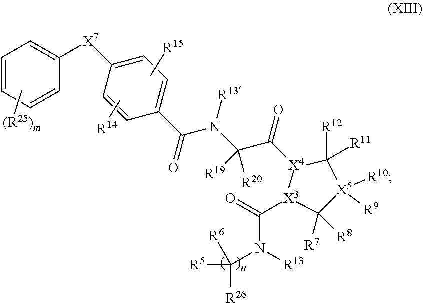

or a pharmaceutically acceptable salt thereof.

205. The compound of any one of embodiments 1-189, wherein the compound is of formula:

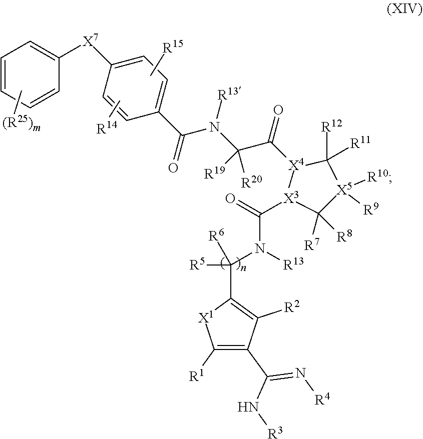

or a pharmaceutically acceptable salt thereof.

206. The compound of any one of embodiments 1-189, wherein the compound is of formula:

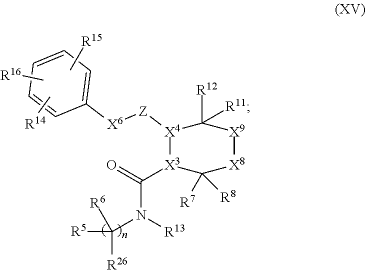

or a pharmaceutically acceptable salt thereof.

207. The compound of any one of embodiments 1-189, wherein the compound is of formula:

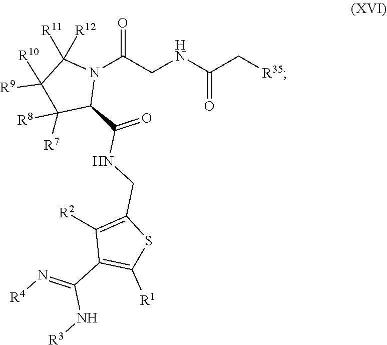

or a pharmaceutically acceptable salt thereof.

208. The compound of any one of embodiments 1-189, wherein the compound is of formula:

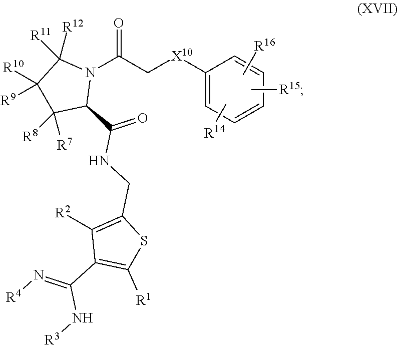

or a pharmaceutically acceptable salt thereof.

209. The compound of any one of embodiments 1-189 wherein the compound is of formula:

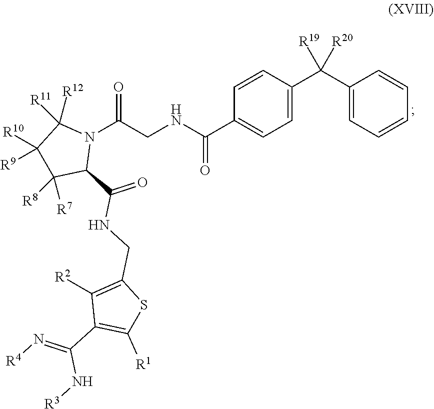

or a pharmaceutically acceptable salt thereof.

210. The compound of any one of embodiments 1-189, wherein the compound is of formula:

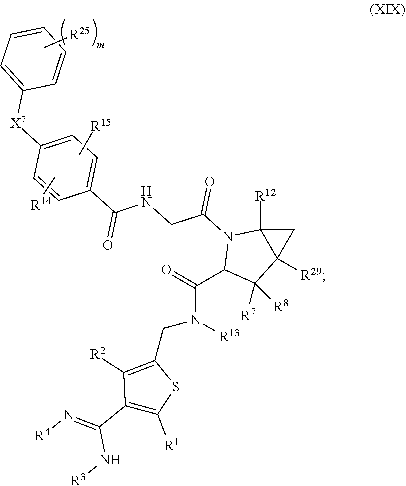

or a pharmaceutically acceptable salt thereof.

211. The compound of any one of embodiments 1-189, wherein the compound is of formula:

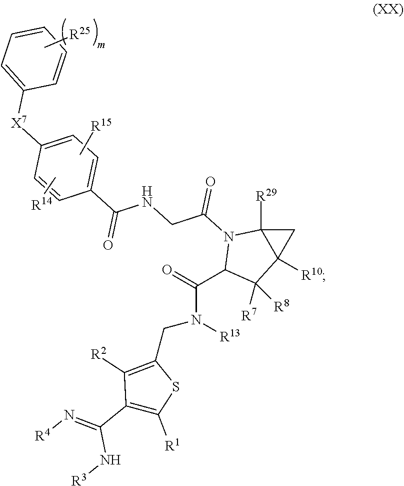

or a pharmaceutically acceptable salt thereof.

212. A compound selected from any one of the compounds of Table 1, or a pharmaceutically acceptable salt thereof.

213. A pharmaceutical composition comprising a compound of any one of embodiments 1-212, or a pharmaceutically acceptable salt thereof, and a pharmaceutically acceptable carrier.

214. A method of treating a complement mediated disorder comprising administering to a subject in need thereof a therapeutically effective amount of a compound of embodiments 1-212 or a pharmaceutically acceptable salt thereof.

215. The method of embodiment 214, wherein the subject is a human.

216. The method of embodiment 214 or 215, wherein the disorder is mediated by C₁s.

217. The method of any one of embodiments 214-216, wherein the disorder is C₃ glomerulopathy.

218. The method of any one of embodiments 214-216, wherein the disorder is an ophthalmic disorder.

219. The method of any one of embodiments 214-216, wherein the disorder is age-related macular degeneration (AMD).

220. The method of any one of embodiments 214-216, wherein the disorder is paroxysmal nocturnal hemoglobinuria (PNH).

221. The method of any one of embodiments 214-216, wherein the disorder is C₃ glomerulonephritis.

222. The method of any one of embodiments 214-216, wherein the disorder is dense deposit disease.

223. The method of any one of embodiments 214-216, wherein the disorder is angioedema.

224. The method of any one of embodiments 214-216, wherein the disorder is hereditary angioedema.

225. The method of any one of embodiments 214-216, wherein the disorder is autoimmune hemolytic anemia.

226. The method of any one of embodiments 214-216, wherein the disorder is cold agglutinin disease.

227. The method of any one of embodiments 214-2167, wherein the disorder is graft rejection.

228. The method of any one of embodiments 214-216, wherein the disorder is selected from hereditary angioedema type 1, hereditary angioedema type 2, trauma, inflammation, sepsis, multiple organ dysfunction syndrome, endotoxemia, end stage renal disease, kidney failure, delayed graft function, ischemic reperfusion injury, neuromyelitis optica, common variable immunodeficiency, antibody-mediated rejection, graft rejection, asthma, allergic asthma, angioneurotic edema, acute ACE-induced angioedema, kidney transplantation, and acute kidney injury.

229. A compound of any one of claims 1-212 or pharmaceutically acceptable salt thereof or a pharmaceutical composition of claim 213 for use in the treatment of a complement mediated disorder.

230. The compound or composition for use of embodiment 231, wherein the subject is a human.

231. The compound or composition for use of embodiment 229 or 230, wherein the disorder is mediated by C₁s.

232. The compound or composition for use of any one of embodiments 229-231, wherein the disorder is C3 glomerulopathy.

233. The compound or composition for use of any one of embodiments 229-231, wherein the disorder is an ophthalmic disorder.

234. The compound or composition for use of any one of embodiments 229-231, wherein the disorder is age-related macular degeneration (AMD).

235. The compound or composition for use of any one of embodiments 229-231, wherein the disorder is paroxysmal nocturnal hemoglobinuria (PNH).

236. The compound or composition for use of any one of embodiments 229-231, wherein the disorder is C3 glomerulonephritis.

237. The compound or composition for use of any one of embodiments 229-231, wherein the disorder is dense deposit disease.

238. The compound or composition for use of any one of embodiments 229-231, wherein the disorder is angioedema.

239. The compound or composition for use of any one of embodiments 229-231, wherein the disorder is hereditary angioedema.

240. The compound or composition for use of any one of embodiments 229-231, wherein the disorder is autoimmune hemolytic anemia.

241. The compound or composition for use of any one of embodiments 229-231, wherein the disorder is cold agglutinin disease.

242. The compound or composition for use of any one of embodiments 229-231, wherein the disorder is graft rejection.

243. The compound or composition for use of any one of embodiments 229-231, wherein the disorder is selected from hereditary angioedema type 1, hereditary angioedema type 2, trauma, inflammation, sepsis, multiple organ dysfunction syndrome, endotoxemia, end stage renal disease, kidney failure, delayed graft function, ischemic reperfusion injury, neuromyelitis optica, common variable immunodeficiency, antibody-mediated rejection, graft rejection, asthma, allergic asthma, angioneurotic edema, acute ACE-induced angioedema, kidney transplantation, and acute kidney injury.

244. Use of a compound of any one of claims 1-213 or its pharmaceutically acceptable salt in the manufacture of a medicament for the treatment of a complement mediated disorder.

245. The use of embodiment 244, wherein the subject is a human.

246. The use of embodiment 244 or 245, wherein the disorder is mediated by C₁s.

247. The use of any one of embodiments 244-246, wherein the disorder is C₃ glomerulopathy.

248. The use of any one of embodiments 244-246, wherein the disorder is an ophthalmic disorder.

249. The use of any one of embodiments 244-246, wherein the disorder is age-related macular degeneration (AMD).

250. The use of any one of embodiments 244-246, wherein the disorder is paroxysmal nocturnal hemoglobinuria (PNH).

251. The use of any one of embodiments 244-246, wherein the disorder is C₃ glomerulonephritis.

252. The use of any one of embodiments 244-246, wherein the disorder is dense deposit disease.

253. The use of any one of embodiments 244-246, wherein the disorder is angioedema.

254. The use of any one of embodiments 244-246, wherein the disorder is hereditary angioedema.

255. The use of any one of embodiments 244-246, wherein the disorder is autoimmune hemolytic anemia.

256. The use of any one of embodiments 244-246, wherein the disorder is cold agglutinin disease.

257. The use of any one of embodiments 244-246, wherein the disorder is graft rejection.

258. The use of any one of embodiments 244-246, wherein the disorder is selected from hereditary angioedema type 1, hereditary angioedema type 2, trauma, inflammation, sepsis, multiple organ dysfunction syndrome, endotoxemia, end stage renal disease, kidney failure, delayed graft function, ischemic reperfusion injury, neuromyelitis optica, common variable immunodeficiency, antibody-mediated rejection, graft rejection, asthma, allergic asthma, angioneurotic edema, acute ACE-induced angioedema, kidney transplantation, and acute kidney injury.

### Pharmaceutical Preparations

Active compounds described herein can be administered to a host in need thereof as the neat chemical, but are more typically administered as a pharmaceutical composition that includes an effective amount for a host, typically a human, in need of such treatment of an active compound as described herein or its pharmaceutically acceptable salt, prodrug, N-oxide, or isolated isomer thereof. Thus, in one embodiment, the disclosure provides pharmaceutical compositions comprising an effective amount of compound or pharmaceutically acceptable salt, prodrug, N-oxide, or isolated isomer thereof together with at least one pharmaceutically acceptable carrier for any of the uses described herein. The pharmaceutical composition may contain a compound or salt as the only active agent, or, in an alternative embodiment, the compound and at least one additional active agent.

An effective amount of an active compound as described herein, or the active compound described herein in combination or alternation with, or preceded by, concomitant with or followed by another active agent, can be used in an amount sufficient to (a) inhibit the progression of a disorder mediated by the complement pathway, including an inflammatory, immune, including an autoimmune, disorder or complement related disorder; (b) cause a regression of an inflammatory, immune, including an autoimmune, disorder or complement related disorder; (c) cause a cure of an inflammatory, immune, including an autoimmune, disorder or complement related disorder; or inhibit or prevent the development of an inflammatory, immune, including an autoimmune, disorder or complement related disorder. Accordingly, an effective amount of an active compound or its salt or composition described herein will provide a sufficient amount of the active agent when administered to a patient to provide a clinical benefit.

The exact amount of the active compound or pharmaceutical composition described herein to be delivered to the host, typically a human, in need thereof, will be determined by the health care provider to achieve the desired clinical benefit.

In certain embodiments, the pharmaceutical composition is in a dosage form that contains from about 0.1 mg to about 2000 mg, from about 10 mg to about 1000 mg, from about 100 mg to about 800 mg, or from about 200 mg to about 600 mg of the active compound and optionally from about 0.1 mg to about 2000 mg, from about 10 mg to about 1000 mg, from about 100 mg to about 800 mg, or from about 200 mg to about 600 mg of an additional active agent in a unit dosage form. Examples are dosage forms with at least about 0.5, 1, 1.5, 2, 2.5, 3, 3.5, 4, 4.5, 5, 10, 15, 20, 25, 50, 75, 100, 150, 200, 250, 300, 350, 400, 450, 500, 550, 600, 650, 700, 750, 800, 900, 1000, 1100, 1200, 1250, 1300, 1400, 1500, or 1600 mg of active compound, or its salt, N-oxide, or prodrug. In one embodiment, the dosage form has at least about 1 mg, 5 mg, 10 mg, 25 mg, 50 mg, 75 mg, 100 mg, 200 mg, 400 mg, 500 mg, 600 mg, 1000 mg, 1200 mg, or 1600 mg of active compound, N-oxide, prodrug, or its salt. The amount of active compound in the dosage form is calculated without reference to the salt. The dosage form can be administered, for example, once a day (q.d.), twice a day (b.i.d.), three times a day (t.i.d.), four times a day (q.i.d.), once every other day (Q2d), once every third day (Q3d), as needed, or any dosage schedule that provides treatment of a disorder described herein.

Compounds disclosed herein or used as described herein may be administered orally, topically, parenterally, by inhalation or spray, sublingually, via implant, including ocular implant, transdermally, via buccal administration, rectally, as an ophthalmic solution, injection, including ocular injection, intravenous, intra-aortal, intracranial, subdermal, intraperitoneal, subcutaneous, transnasal, sublingual, intrathecal, or rectal or by other means, in dosage unit formulations containing conventional pharmaceutically acceptable carriers. For ocular delivery, the compound can be administered, as desired, for example, as a solution, suspension, or other formulation via intravitreal, intrastromal, intracameral, sub-tenon, sub-retinal, retro-bulbar, peribulbar, suprachorodial, subchorodial, chorodial, conjunctival, subconjunctival, episcleral, periocular, transscleral, retrobulbar, posterior juxtascleral, circumcorneal, or tear duct injections, or through a mucus, mucin, or a mucosal barrier, in an immediate or controlled release fashion or via an ocular device, injection, or topically administered formulation, for example, a solution or suspension provided as an eye drop.

The pharmaceutical composition may be formulated as any pharmaceutically useful form, e.g., as an aerosol, a cream, a gel, a gel cap, a pill, a microparticle, a nanoparticle, an injection or infusion solution, a capsule, a tablet, a syrup, a transdermal patch, a subcutaneous patch, a dry powder, an inhalation formulation, in a medical device, suppository, buccal, or sublingual formulation, parenteral formulation, or an ophthalmic solution or suspension. Some dosage forms, such as tablets and capsules, are subdivided into suitably sized unit doses containing appropriate quantities of the active components, e.g., an effective amount to achieve the desired purpose.

Pharmaceutical compositions, and methods of manufacturing such compositions, suitable for administration as contemplated herein are known in the art. Examples of known techniques include, for example, U.S. Pat. Nos. 4,983,593; 5,013,557; 5,456,923; 5,576,025; 5,723,269; 5,858,411; 6,254,889; 6,303,148; 6,395,302; 6,497,903; 7,060,296; 7,078,057; 7,404,828; 8,202,912; 8,257,741; 8,263,128; 8,337,899; 8,431,159; 9,028,870; 9,060,938; 9,211,261; 9,265,731; 9,358,478; and 9,387,252; incorporated by reference herein.

The pharmaceutical compositions contemplated here can optionally include a carrier. Carriers must be of sufficiently high purity and sufficiently low toxicity to render them suitable for administration to the patient being treated. The carrier can be inert or it can possess pharmaceutical benefits of its own. The amount of carrier employed in conjunction with the compound is sufficient to provide a practical quantity of material for administration per unit dose of the compound. Classes of carriers include, but are not limited to binders, buffering agents, coloring agents, diluents, disintegrants, emulsifiers, fillers, flavorants, glidents, lubricants, pH modifiers, preservatives, stabilizers, surfactants, solubilizers, tableting agents, and wetting agents. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin; talc, and vegetable oils.

Examples of other matrix materials, fillers, or diluents include lactose, mannitol, xylitol, microcrystalline cellulose, calcium diphosphate, and starch. Examples of surface active agents include sodium lauryl sulfate and polysorbate 80.

Examples of drug complexing agents or solubilizers include the polyethylene glycols, caffeine, xanthene, gentisic acid and cylodextrins.

Examples of disintegrants include sodium starch gycolate, sodium alginate, carboxymethyl cellulose sodium, methyl cellulose, colloidal silicon dioxide, and croscarmellose sodium.

Examples of binders include methyl cellulose, microcrystalline cellulose, starch, and gums such as guar gum, and tragacanth.

Examples of lubricants include magnesium stearate and calcium stearate.

Examples of pH modifiers include acids such as citric acid, acetic acid, ascorbic acid, lactic acid, aspartic acid, succinic acid, phosphoric acid, and the like; bases such as sodium acetate, potassium acetate, calcium oxide, magnesium oxide, trisodium phosphate, sodium hydroxide, calcium hydroxide, aluminum hydroxide, and the like, and buffers generally comprising mixtures of acids and the salts of said acids. Optional other active agents may be included in a pharmaceutical composition, which do not substantially interfere with the activity of the compound of the present disclosure.

In certain embodiments, the pharmaceutical composition for administration further includes a compound or salt of Formula I, II, III, IV, V, VI, VII, VIII, IX, X, XI, XII, XIII, XIV, XV, XVI, XVII, XVIII, XIX, or XX, and optionally comprises one or more of a phosphoglyceride; phosphatidylcholine; dipalmitoyl phosphatidylcholine (DPPC); dioleylphosphatidyl ethanolamine (DOPE); dioleyloxypropyltriethylammonium (DOTMA); dioleoylphosphatidylcholine; cholesterol; cholesterol ester; diacylglycerol; diacylglycerolsuccinate; diphosphatidyl glycerol (DPPG); hexanedecanol; fatty alcohol such as polyethylene glycol (PEG); polyoxyethylene-9-lauryl ether; a surface active fatty acid, such as palmitic acid or oleic acid; fatty acid; fatty acid monoglyceride; fatty acid diglyceride; fatty acid amide; sorbitan trioleate (SPAN®85) glycocholate; sorbitan monolaurate (SPAN®20); polysorbate 20 (TWEEN®20); polysorbate 60 (TWEEN®60); polysorbate 65 (TWEEN®65); polysorbate 80 (TWEEN®80); polysorbate 85 (TWEEN®85); polyoxyethylene monostearate; surfactin; a poloxomer; a sorbitan fatty acid ester such as sorbitan trioleate; lecithin; lysolecithin; phosphatidylserine; phosphatidylinositol; sphingomyelin; phosphatidylethanolamine (cephalin); cardiolipin; phosphatidic acid; cerebroside; dicetylphosphate; dipalmitoylphosphatidylglycerol; stearylamine; dodecylamine; hexadecyl-amine; acetyl palmitate; glycerol ricinoleate; hexadecyl sterate; isopropyl myristate; tyloxapol; poly(ethylene glycol)5000-phosphatidylethanolamine; poly(ethylene glycol)400-monostearate; phospholipid; synthetic and/or natural detergent having high surfactant properties; deoxycholate; cyclodextrin; chaotropic salt; ion pairing agent; glucose, fructose, galactose, ribose, lactose, sucrose, maltose, trehalose, cellbiose, mannose, xylose, arabinose, glucoronic acid, galactoronic acid, mannuronic acid, glucosamine, galatosamine, and neuramic acid; pullulan, cellulose, microcrystalline cellulose, hydroxypropyl methylcellulose (HPMC), hydroxycellulose (HC), methylcellulose (MC), dextran, cyclodextran, glycogen, hydroxyethylstarch, carageenan, glycon, amylose, chitosan, N,O-carboxylmethylchitosan, algin and alginic acid, starch, chitin, inulin, konjac, glucommannan, pustulan, heparin, hyaluronic acid, curdlan, and xanthan, mannitol, sorbitol, xylitol, erythritol, maltitol, and lactitol, a pluronic polymer, polyethylene, polycarbonate (e.g., poly(1,3-dioxan-2one)), polyanhydride (e.g., poly(sebacic anhydride)), polypropylfumerate, polyamide (e.g., polycaprolactam), polyacetal, polyether, polyester (e.g., polylactide, polyglycolide, polylactide-co-glycolide, polycaprolactone, polyhydroxyacid (e.g., poly((p-hydroxyalkanoate))), poly(orthoester), polycyanoacrylate, polyvinyl alcohol, polyurethane, polyphosphazene, polyacrylate, polymethacrylate, polyurea, polystyrene, and polyamine, polylysine, polylysine-PEG copolymer, and poly(ethyleneimine), poly(ethylene imine)-PEG copolymer, glycerol monocaprylocaprate, propylene glycol, Vitamin E TPGS (also known as d-α-Tocopheryl polyethylene glycol 1000 succinate), gelatin, titanium dioxide, polyvinylpyrrolidone (PVP), hydroxypropyl methyl cellulose (HPMC), hydroxypropyl cellulose (HPC), methyl cellulose (MC), block copolymers of ethylene oxide and propylene oxide (PEO/PPO), polyethyleneglycol (PEG), sodium carboxymethylcellulose (NaCMC), and hydroxypropylmethyl cellulose acetate succinate (HPMCAS).

In some embodiments, the pharmaceutical preparation may include polymers for controlled delivery of the described compounds, including, but not limited to pluronic polymers, polyesters (e.g., polylactic acid, poly(lactic-co-glycolic acid), polycaprolactone, polyvalerolactone, poly(1,3-dioxan-2one)); polyanhydrides (e.g., poly(sebacic anhydride)); polyethers (e.g., polyethylene glycol); polyurethanes; polymethacrylates; polyacrylates; and polycyanoacrylates.

In some embodiments, polymers may be modified with polyethylene glycol (PEG), with a carbohydrate, and/or with acyclic polyacetals derived from polysaccharides. See, e.g., Papisov, 2001, ACS Symposium Series, 786:301, incorporated by reference herein.

The compounds of the present disclosure can be formulated as particles. In one embodiment the particles are, or include, microparticles. In an alternative embodiment, the particles are or include nanoparticles.

In an additional alternative embodiment, common techniques for preparing particles include, but are not limited to, solvent evaporation, solvent removal, spray drying, phase inversion, coacervation, and low temperature casting. Suitable methods of particle formulation are briefly described herein. Pharmaceutically acceptable excipients, including pH modifying agents, disintegrants, preservatives, and antioxidants, can optionally be incorporated into the particles during particle formation.

In one embodiment, the particles are derived through a solvent evaporation method. In this method, a compound described herein (or polymer matrix and one or more compounds described herein) is dissolved in a volatile organic solvent, such as methylene chloride. The organic solution containing a compound described herein is then suspended in an aqueous solution that contains a surface active agent such as poly(vinyl alcohol). The resulting emulsion is stirred until most of the organic solvent evaporated, leaving solid nanoparticles or microparticles. The resulting nanoparticles or microparticles are washed with water and dried overnight in a lyophilizer (under vacuum, with or without heat). Nanoparticles with different sizes and morphologies can be obtained by this method.

Pharmaceutical compositions which contain labile polymers, such as certain polyanhydrides, may degrade during the fabrication process due to the presence of water. For these polymers, methods which are performed in completely or substantially anhydrous organic solvents can be used to make the particles.

Solvent removal can also be used to prepare particles from a compound that is hydrolytically unstable. In this method, the compound (or polymer matrix and one or more compounds) is dispersed or dissolved in a volatile organic solvent such as methylene chloride. This mixture is then suspended by stirring in an organic oil (such as silicon oil) to form an emulsion. Solid particles form from the emulsion, which can subsequently be isolated from the supernatant. The external morphology of spheres produced with this technique is highly dependent on the identity of the drug.

In one embodiment, an active compound as described herein is administered to a patient in need thereof as particles formed by solvent removal. In another embodiment, the present disclosure provides particles formed by solvent removal comprising a compound of the present disclosure and one or more pharmaceutically acceptable excipients as defined herein. In another embodiment, the particles formed by solvent removal comprise a compound of the present disclosure and an additional therapeutic agent. In a further embodiment, the particles formed by solvent removal comprise a compound of the present disclosure, an additional therapeutic agent, and one or more pharmaceutically acceptable excipients. In another embodiment, any of the described particles formed by solvent removal can be formulated into a tablet, and then coated to form a coated tablet. In an alternative embodiment, the particles formed by solvent removal are formulated into a tablet but the tablet is uncoated.

In one embodiment, the particles are derived by spray drying. In this method, a compound (or polymer matrix and one or more compounds) is dissolved in an organic solvent such as methylene chloride. The solution is pumped through a micronizing nozzle driven by a flow of compressed gas, and the resulting aerosol is suspended in a heated cyclone of air, allowing the solvent to evaporate from the micro droplets, forming particles. Microparticles and nanoparticles can be obtained using this method.

In one embodiment, an active compound as described herein is administered to a patient in need thereof as a spray dried dispersion (SDD). In another embodiment, the present disclosure provides a spray dried dispersion (SDD) comprising a compound of the present disclosure and one or more pharmaceutically acceptable excipients as defined herein. In another embodiment, the SDD comprises a compound of the present disclosure and an additional therapeutic agent. In a further embodiment, the SDD comprises a compound of the present disclosure, an additional therapeutic agent, and one or more pharmaceutically acceptable excipients. In another embodiment, any of the described spray dried dispersions can be coated to form a coated tablet. In an alternative embodiment, the spray dried dispersion is formulated into a tablet but the tablet is uncoated.

Particles can be formed from the active compound as described herein using a phase inversion method. In this method, the compound (or polymer matrix and one or more active compounds) is dissolved in a suitable solvent, and the solution is poured into a strong non-solvent for the compound to spontaneously produce, under favorable conditions, microparticles or nanoparticles. The method can be used to produce nanoparticles in a wide range of sizes, including, for example, from nanoparticles to microparticles, typically possessing a narrow particle size distribution.

In one embodiment, an active compound as described herein is administered to a patient in need thereof as particles formed by phase inversion. In another embodiment, the present disclosure provides particles formed by phase inversion comprising a compound of the present disclosure and one or more pharmaceutically acceptable excipients as defined herein. In another embodiment the particles formed by phase inversion comprise a compound of the present disclosure and an additional therapeutic agent. In a further embodiment the particles formed by phase inversion comprise a compound of the present disclosure, an additional therapeutic agent, and one or more pharmaceutically acceptable excipients. In another embodiment, any of the described particles formed by phase inversion can be formulated into a tablet and then coated to form a coated tablet. In an alternative embodiment, the particles formed by phase inversion are formulated into a tablet, but the tablet is uncoated.

Techniques for particle formation using coacervation are known in the art, for example, as described in GB-B-929 406; GB-B-929 40 1; and U.S. Pat. Nos. 3,266,987; 4,794,000; and 4,460,563. Coacervation involves the separation of a compound (or polymer matrix and one or more compounds) solution into two immiscible liquid phases. One phase is a dense coacervate phase, which contains a high concentration of the compound, while the second phase contains a low concentration of the compound. Within the dense coacervate phase, the compound forms nanoscale or microscale droplets, which harden into particles. Coacervation may be induced by a temperature change, addition of a non-solvent or addition of a micro-salt (simple coacervation), or by the addition of another polymer thereby forming an interpolymer complex (complex coacervation).

In one embodiment, an active compound as described herein is administered to a patient in need thereof as particles formed by coacervation. In another embodiment, the present disclosure provides particles formed by coacervation comprising a compound of the present disclosure and one or more pharmaceutically acceptable excipients as defined herein. In another embodiment, the particles formed by coacervation comprise a compound of the present disclosure and an additional therapeutic agent. In a further embodiment, the particles formed by coacervation comprise a compound of the present disclosure, an additional therapeutic agent, and one or more pharmaceutically acceptable excipients. In another embodiment, any of the described particles formed by coacervation can be formulated into a tablet and then coated to form a coated tablet. In an alternative embodiment, the particles formed by coacervation are formulated into a tablet, but the tablet is uncoated.

Methods for very low temperature casting of controlled release microspheres are described in U.S. Pat. No. 5,019,400 to Gombotz et al. In this method, the compound is dissolved in a solvent. The mixture is then atomized into a vessel containing a liquid non-solvent at a temperature below the freezing point of the drug solution which freezes the compound droplets. As the droplets and non-solvent for the compound are warmed, the solvent in the droplets thaws and is extracted into the non-solvent, hardening the microspheres.

In one embodiment, a compound of the present disclosure is administered to a patient in need thereof as particles formed by low temperature casting. In another embodiment the present disclosure provides particles formed by low temperature casting comprising a compound of the present disclosure and one or more pharmaceutically acceptable excipients as defined herein. In another embodiment, the particles formed by low temperature casting comprise a compound of the present disclosure and an additional therapeutic agent. In a further embodiment, the particles formed by low temperature casting comprise a compound of the present disclosure, an additional therapeutic agent, and one or more pharmaceutically acceptable excipients. In another embodiment, any of the described particles formed by low temperature casting can be formulated into a tablet and then coated to form a coated tablet. In an alternative embodiment, the particles formed by low temperature casting are formulated into a tablet, but the tablet is uncoated.

In one aspect of the present disclosure, an effective amount of an active compound as described herein is incorporated into a nanoparticle, e.g., for convenience of delivery and/or extended release delivery. The use of materials in nanoscale provides one the ability to modify fundamental physical properties such as solubility, diffusivity, blood circulation half-life, drug release characteristics, and/or immunogenicity. A number of nanoparticle-based therapeutic and diagnostic agents have been developed for the treatment of cancer, diabetes, pain, asthma, allergy, and infections. These nanoscale agents may provide more effective and/or more convenient routes of administration, lower therapeutic toxicity, extend the product life cycle, and ultimately reduce health-care costs. As therapeutic delivery systems, nanoparticles can allow targeted delivery and controlled release.

In addition, nanoparticle-based compound delivery can be used to release compounds at a sustained rate and thus lower the frequency of administration, deliver drugs in a targeted manner to minimize systemic side effects, or deliver two or more drugs simultaneously for combination therapy to generate a synergistic effect and suppress drug resistance. A number of nanotechnology-based therapeutic products have been approved for clinical use. Among these products, liposomal drugs and polymer-based conjugates account for a large proportion of the products. See Zhang, L., et al., Nanoparticles in Medicine: Therapeutic Applications and Developments, Clin. Pharm. and Ther., 83(5):761-769, 2008.

Methods for producing nanoparticles are known in the art. For example, see Muller, R. H., et al., Solid lipid nanoparticles (SLN) for controlled drug delivery—a review of the state of the art, Eur. H. Pharm. Biopharm., 50:161-177, 2000; U.S. Pat. No. 8,691,750 to Consien et al.; WO 2012/145801 to Kanwar; U.S. Pat. No. 8,580,311 to Armes, S. et al.; Petros, R. A. and DeSimone, J. M., Strategies in the design of nanoparticles for therapeutic applications, Nature Reviews/Drug Discovery, vol. 9:615-627, 2010; U.S. Pat. Nos. 8,465,775; 8,444,899; 8,420,124; 8,263,129; 8,158,728; 8,268,446; Pellegrino et al., 2005, Small, 1:48; Murray et al., 2000, Ann. Rev. Mat. Sci., 30:545; and Trindade et al., 2001, Chem. Mat., 13:3843; all incorporated herein by reference. Additional methods have been described in the literature (see, e.g., Doubrow, Ed., “Microcapsules and Nanoparticles in Medicine and Pharmacy,” CRC Press, Boca Raton, 1992; Mathiowitz et al., 1987, J. Control. Release, 5:13; Mathiowitz et al., 1987, Reactive Polymers, 6:275; and Mathiowitz et al., 1988, J. Appl. Polymer Sci., 35:755; U.S. Pat. Nos. 5,578,325 and 6,007,845; P. Paolicelli et al., “Surface-modified PLGA-based Nanoparticles that can Efficiently Associate and Deliver Virus-like Particles” Nanomedicine. 5(6):843-853 (2010)), U.S. Pat. No. 5,543,158 to Gref et al., or WO publication WO2009/051837 by Von Andrian et al.; Zauner et al., 1998, Adv. Drug Del. Rev., 30:97; and Kabanov et al., 1995, Bioconjugate Chem., 6:7; (PEI; Boussif et al., 1995, Proc. Natl. Acad. Sci., USA, 1995, 92:7297), and poly(amidoamine) dendrimers (Kukowska-Latallo et al., 1996, Proc. Natl. Acad. Sci., USA, 93:4897; Tang et al., 1996, Bioconjugate Chem., 7:703; and Haensler et al., 1993, Bioconjugate Chem., 4:372; Putnam et al., 1999, Macromolecules, 32:3658; Barrera et al., 1993, J. Am. Chem. Soc., 115:11010; Kwon et al., 1989, Macromolecules, 22:3250; Lim et al., 1999, J. Am. Chem. Soc., 121:5633; and Zhou et al., 1990, Macromolecules, 23:3399). Examples of these polyesters include poly(L-lactide-co-L-lysine) (Barrera et al., 1993, J. Am. Chem. Soc., 115:11010), poly(serine ester) (Zhou et al., 1990, Macromolecules, 23:3399), poly(4-hydroxy-L-proline ester) (Putnam et al., 1999, Macromolecules, 32:3658; and Lim et al., 1999, J. Am. Chem. Soc., 121:5633), and poly(4-hydroxy-L-proline ester) (Putnam et al., 1999, Macromolecules, 32:3658; and Lim et al., 1999, J. Am. Chem. Soc., 121:5633; U.S. Pat. Nos. 6,123,727; 5,804,178; 5,770,417; 5,736,372; 5,716,404; 6,095,148; 5,837,752; 5,902,599; 5,696,175; 5,514,378; 5,512,600; 5,399,665; 5,019,379; 5,010,167; 4,806,621; 4,638,045; and U.S. Pat. No. 4,946,929; Wang et al., 2001, J. Am. Chem. Soc., 123:9480; Lim et al., 2001, J. Am. Chem. Soc., 123:2460; Langer, 2000, Acc. Chem. Res., 33:94; Langer, 1999, J. Control. Release, 62:7; and Uhrich et al., 1999, Chem. Rev., 99:3181; Concise Encyclopedia of Polymer Science and Polymeric Amines and Ammonium Salts, Ed. by Goethals, Pergamon Press, 1980; Principles of Polymerization by Odian, John Wiley & Sons, Fourth Edition, 2004; Contemporary Polymer Chemistry by Allcock et al., Prentice-Hall, 1981; Deming et al., 1997, Nature, 390:386; and in U.S. Pat. Nos. 6,506,577, 6,632,922, 6,686,446, and 6,818,732; C. Astete et al., “Synthesis and characterization of PLGA nanoparticles” J. Biomater. Sci. Polymer Edn, Vol. 17, No. 3, pp. 247-289 (2006); K. Avgoustakis “Pegylated Poly(Lactide) and Poly(Lactide-Co-Glycolide) Nanoparticles: Preparation, Properties and Possible Applications in Drug Delivery” Current Drug Delivery 1:321-333 (2004); C. Reis et al., “Nanoencapsulation I. Methods for preparation of drug-loaded polymeric nanoparticles” Nanomedicine 2:8-21 (2006); P. Paolicelli et al., “Surface-modified PLGA-based Nanoparticles that can Efficiently Associate and Deliver Virus-like Particles” Nanomedicine. 5(6):843-853 (2010); and U.S. Pat. No. 6,632,671 to Unger Oct. 14, 2003, all incorporated herein by reference.

In one embodiment, the polymeric particle is between about 0.1 nm to about 10000 nm, between about 1 nm to about 1000 nm, between about 10 nm and 1000 nm, between about 1 and 100 nm, between about 1 and 10 nm, between about 1 and 50 nm, between about 100 nm and 800 nm, between about 400 nm and 600 nm, or about 500 nm. In one embodiment, the micro-particles are no more than about 0.1 nm, 0.5 nm, 1.0 nm, 5.0 nm, 10 nm, 25 nm, 50 nm, 75 nm, 100 nm, 150 nm, 200 nm, 250 nm, 300 nm, 400 nm, 450 nm, 500 nm, 550 nm, 600 nm, 650 nm, 700 nm, 750 nm, 800 nm, 850 nm, 900 nm, 950 nm, 1000 nm, 1250 nm, 1500 nm, 1750 nm, or 2000 nm. In some embodiments, a compound described herein may be covalently coupled to a polymer used in the nanoparticle, for example a polystyrene particle, PLGA particle, PLA particle, or other nanoparticle.

The pharmaceutical compositions according to the disclosure can be formulated for oral administration. These compositions can contain any amount of active compound that achieves the desired result, for example, between 0.1 and 99 weight % (wt. %) of the compound, and usually at least about 5 wt. % of the compound. Some embodiments contain at least about 10%, 15%, 20%, 25 wt. % to about 50 wt. % or from about 5 wt. % to about 75 wt. % of the compound.

Pharmaceutical compositions suitable for rectal administration are typically presented as unit dose suppositories. These may be prepared by admixing the active compound with one or more conventional solid carriers, for example, cocoa butter, and then shaping the resulting mixture.

Pharmaceutical compositions suitable for topical application to the skin preferably take the form of an ointment, cream, lotion, paste, gel, spray, aerosol, or oil. Carriers which may be used include petroleum jelly, lanoline, polyethylene glycols, alcohols, transdermal enhancers, and combinations of two or more thereof.

Pharmaceutical compositions suitable for transdermal administration may be presented as discrete patches adapted to remain in intimate contact with the epidermis of the recipient for a prolonged period of time. Pharmaceutical compositions suitable for transdermal administration may also be delivered by iontophoresis (see, for example, Pharmaceutical Research 3 (6):318 (1986)) and typically take the form of an optionally buffered aqueous solution of the active compound. In one embodiment, microneedle patches or devices are provided for delivery of drugs across or into biological tissue, particularly the skin. The microneedle patches or devices permit drug delivery at clinically relevant rates across or into skin or other tissue barriers, with minimal or no damage, pain, or irritation to the tissue.

Pharmaceutical compositions suitable for administration to the lungs can be delivered by a wide range of passive breath driven and active power driven single/-multiple dose dry powder inhalers (DPI). The devices most commonly used for respiratory delivery include nebulizers, metered-dose inhalers, and dry powder inhalers. Several types of nebulizers are available, including jet nebulizers, ultrasonic nebulizers, and vibrating mesh nebulizers. Selection of a suitable lung delivery device depends on parameters, such as nature of the drug and its formulation, the site of action, and pathophysiology of the lung.

Additional non-limiting examples of inhalation drug delivery devices and methods include, for example, U.S. Pat. No. 7,383,837 titled “Inhalation Device” (SmithKline Beecham Corporation); WO/2006/033584 titled “Powder Inhaler” (Glaxo SmithKline Pharmaceuticals SA); WO/2005/044186 titled “Inhalable Pharmaceutical Formulations Employing Desiccating Agents and Methods of Administering the Same” (Glaxo Group Ltd and SmithKline Beecham Corporation); U.S. Pat. No. 9,095,670 titled “Inhalation Device and Method of Dispensing Medicament”, U.S. Pat. No. 8,205,611 titled “Dry Powder Inhaler” (Astrazeneca AB); WO/2013/038170 titled “Inhaler” (Astrazeneca AB and Astrazeneca UK Ltd.); US/2014/0352690 titled “Inhalation Device with Feedback System”, U.S. Pat. No. 8,910,625 and US/2015/0165137 titled “Inhalation Device for Use in Aerosol Therapy” (Vectura GmbH); U.S. Pat. No. 6,948,496 titled “Inhalers”, US/2005/0152849 titled “Powders Comprising Anti-adherent Materials for Use in Dry Powder Inhalers”, U.S. Pat. Nos. 6,582,678, 8,137,657, US/2003/0202944, and US/2010/0330188 titled “Carrier Particles for Use in Dry Powder Inhalers”, U.S. Pat. No. 6,221,338 titled “Method of Producing Particles for Use in Dry Powder Inhalers”, U.S. Pat. No. 6,989,155 titled “Powders”, US/2007/0043030 titled “Pharmaceutical Compositions for Treating Premature Ejaculation by Pulmonary Inhalation”, U.S. Pat. No. 7,845,349 titled “Inhaler”, US/2012/0114709 and U.S. Pat. No. 8,101,160 titled “Formulations for Use in Inhaler Devices”, US/2013/0287854 titled “Compositions and Uses”, US/2014/0037737 and U.S. Pat. No. 8,580,306 titled “Particles for Use in a Pharmaceutical Composition”, US/2015/0174343 titled “Mixing Channel for an Inhalation Device”, U.S. Pat. No. 7,744,855 and US/2010/0285142 titled “Method of Making Particles for Use in a Pharmaceutical Composition”, U.S. Pat. No. 7,541,022, US/2009/0269412, and US/2015/0050350 titled “Pharmaceutical Formulations for Dry Powder Inhalers” (Vectura Limited).

Many methods and devices for drug delivery to the eye are known in the art. Non-limiting examples are described in the following patents and patent applications (fully incorporated herein by reference): U.S. Pat. No. 8,192,408 titled “Ocular trocar assembly” (Psivida Us, Inc.); U.S. Pat. No. 7,585,517 titled “Transcleral delivery” (Macusight, Inc.); U.S. Pat. Nos. 5,710,182 and 5,795,913 titled “Ophthalmic composition” (Santen OY); U.S. Pat. No. 8,663,639 titled “Formulations for treating ocular diseases and conditions”, U.S. Pat. No. 8,486,960 titled “Formulations and methods for vascular permeability-related diseases or conditions”, U.S. Pat. Nos. 8,367,097 and 8,927,005 titled “Liquid formulations for treatment of diseases or conditions”, U.S. Pat. No. 7,455,855 titled “Delivering substance and drug delivery system using the same” (Santen Pharmaceutical Co., Ltd.); WO/2011/050365 titled “Conformable Therapeutic Shield For Vision and Pain” and WO/2009/145842 titled “Therapeutic Device for Pain Management and Vision” (Forsight Labs, LLC); U.S. Pat. Nos. 9,066,779 8,623,395 titled “Implantable therapeutic device”, WO/2014/160884 titled “Ophthalmic Implant for Delivering Therapeutic Substances”, U.S. Pat. Nos. 8,399,006, 8,277,830, 8,795,712, 8,808,727, 8,298,578, and WO/2010/088548 titled “Posterior segment drug delivery”, WO/2014/152959 and US20140276482 titled “Systems for Sustained Intraocular Delivery of Low Solubility Compounds from a Port Delivery System Implant”, U.S. Pat. Nos. 8,905,963 and 9,033,911 titled “Injector apparatus and method for drug delivery”, WO/2015/057554 titled “Formulations and Methods for Increasing or Reducing Mucus”, U.S. Pat. Nos. 8,715,712 and 8,939,948 titled “Ocular insert apparatus and methods”, WO/2013/116061 titled “Insertion and Removal Methods and Apparatus for Therapeutic Devices”, WO/2014/066775 titled “Ophthalmic System for Sustained Release of Drug to the Eye”, WO/2015/085234 and WO/2012/019176 titled “Implantable Therapeutic Device”, WO/2012/065006 titled “Methods and Apparatus to determine Porous Structures for Drug Delivery”, WO/2010/141729 titled “Anterior Segment Drug Delivery”, WO/2011/050327 titled “Corneal Denervation for Treatment of Ocular Pain”, WO/2013/022801 titled “Small Molecule Delivery with Implantable Therapeutic Device”, WO/2012/019047 titled “Subconjunctival Implant for Posterior Segment Drug Delivery”, WO/2012/068549 titled “Therapeutic Agent Formulations for Implanted Devices”, WO/2012/019139 titled “Combined Delivery Methods and Apparatus”, WO/2013/040426 titled “Ocular Insert Apparatus and Methods”, WO/2012/019136 titled “Injector Apparatus and Method for Drug Delivery”, and WO/2013/040247 titled “Fluid Exchange Apparatus and Methods” (ForSight Vision4, Inc.).

Additional non-limiting examples of how to deliver the active compounds are provided in WO/2015/085251 titled “Intracameral Implant for Treatment of an Ocular Condition” (Envisia Therapeutics, Inc.); WO/2011/008737 titled “Engineered Aerosol Particles, and Associated Methods”, WO/2013/082111 titled “Geometrically Engineered Particles and Methods for Modulating Macrophage or Immune Responses”, WO/2009/132265 titled “Degradable compounds and methods of use thereof, particularly with particle replication in non-wetting templates”, WO/2010/099321 titled “Interventional drug delivery system and associated methods”, WO/2008/100304 titled “Polymer particle composite having high fidelity order, size, and shape particles”, WO/2007/024323 titled “Nanoparticle fabrication methods, systems, and materials” (Liquidia Technologies, Inc. and the University of North Carolina at Chapel Hill); WO/2010/009087 titled “lontophoretic Delivery of a Controlled-Release Formulation in the Eye”, (Liquidia Technologies, Inc. and Eyegate Pharmaceuticals, Inc.) and WO/2009/132206 titled “Compositions and Methods for Intracellular Delivery and Release of Cargo”, WO/2007/133808 titled “Nano-particles for cosmetic applications”, WO/2007/056561 titled “Medical device, materials, and methods”, WO/2010/065748 titled “Method for producing patterned materials”, and WO/2007/081876 titled “Nanostructured surfaces for biomedical/biomaterial applications and processes thereof” (Liquidia Technologies, Inc.).

Additional non-limiting examples of methods and devices for drug delivery to the eye include, for example, WO2011/106702 and U.S. Pat. No. 8,889,193 titled “Sustained delivery of therapeutic agents to an eye compartment”, WO2013/138343 and U.S. Pat. No. 8,962,577 titled “Controlled release formulations for the delivery of HIF-1 inhibitors”, WO/2013/138346 and US2013/0272994 titled “Non-Linear Multiblock Copolymer-Drug Conjugates for the Delivery of Active Agents”, WO2005/072710 and U.S. Pat. No. 8,957,034 titled “Drug and Gene Carrier Particles that Rapidly Move Through Mucus Barriers”, WO2008/030557, US2010/0215580, US2013/0164343 titled “Compositions and Methods for Enhancing Transport Through Mucous”, WO2012/061703, US2012/0121718, and US2013/0236556 titled “Compositions and Methods Relating to Reduced Mucoadhesion”, WO2012/039979 and US2013/0183244 titled “Rapid Diffusion of Large Polymeric Nanoparticles in the Mammalian Brain”, WO2012/109363 and US2013/0323313 titled “Mucus Penetrating Gene Carriers”, WO 2013/090804 and US2014/0329913 titled “Nanoparticles with enhanced mucosal penetration or decreased inflammation”, WO2013/110028 titled “Nanoparticle formulations with enhanced mucosal penetration”, WO2013/166498 and US2015/0086484 titled “Lipid-based drug carriers for rapid penetration through mucus linings” (The Johns Hopkins University); WO2013/166385 titled “Pharmaceutical Nanoparticles Showing Improved Mucosal Transport”, US2013/0323179 titled “Nanocrystals, Compositions, And Methods that Aid Particle Transport in Mucus” (The Johns Hopkins University and Kala Pharmaceuticals, Inc.); WO/2015/066444 titled “Compositions and methods for ophthalmic and/or other applications”, WO/2014/020210 and WO/2013/166408 titled “Pharmaceutical nanoparticles showing improved mucosal transport” (Kala Pharmaceuticals, Inc.); U.S. Pat. No. 9,022,970 titled “Ophthalmic injection device including dosage control device”, WO/2011/153349 titled “Ophthalmic compositions comprising pbo-peo-pbo block copolymers”, WO/2011/140203 titled “Stabilized ophthalmic galactomannan formulations”, WO/2011/068955 titled “Ophthalmic emulsion”, WO/2011/037908 titled “Injectable aqueous ophthalmic composition and method of use therefor”, US2007/0149593 titled “Pharmaceutical Formulation for Delivery of Receptor Tyrosine Kinase Inhibiting (RTKi) Compounds to the Eye”, and U.S. Pat. No. 8,632,809 titled “Water insoluble polymer matrix for drug delivery” (Alcon, Inc.).

Additional non-limiting examples of drug delivery devices and methods include, for example, US 2009/0203709 titled “Pharmaceutical Dosage Form For Oral Administration Of Tyrosine Kinase Inhibitor” (Abbott Laboratories); US 2005/0009910 titled “Delivery of an active drug to the posterior part of the eye via subconjunctival or periocular delivery of a prodrug”, US 20130071349 titled “Biodegradable polymers for lowering intraocular pressure”, U.S. Pat. No. 8,481,069 titled “Tyrosine kinase microspheres”, U.S. Pat. No. 8,465,778 titled “Method of making tyrosine kinase microspheres”, U.S. Pat. No. 8,409,607 titled “Sustained release intraocular implants containing tyrosine kinase inhibitors and related methods”, U.S. Pat. No. 8,512,738 and US 2014/0031408 titled “Biodegradable intravitreal tyrosine kinase implants”, US 2014/0294986 titled “Microsphere Drug Delivery System for Sustained Intraocular Release”, U.S. Pat. No. 8,911,768 titled “Methods For Treating Retinopathy With Extended Therapeutic Effect” (Allergan, Inc.); U.S. Pat. No. 6,495,164 titled “Preparation of injectable suspensions having improved injectability” (Alkermes Controlled Therapeutics, Inc.); WO 2014/047439 titled “Biodegradable Microcapsules Containing Filling Material” (Akina, Inc.); WO 2010/132664 titled “Compositions And Methods For Drug Delivery” (Baxter International Inc. Baxter Healthcare SA); US 2012/0052041 titled “Polymeric nanoparticles with enhanced drugloading and methods of use thereof” (The Brigham and Women's Hospital, Inc.); US 2014/0178475, US 2014/0248358, and US20140249158 titled “Therapeutic Nanoparticles Comprising a Therapeutic Agent and Methods of Making and Using Same” (BIND Therapeutics, Inc.); U.S. Pat. No. 5,869,103 titled “Polymer microparticles for drug delivery” (Danbiosyst UK Ltd.); U.S. Pat. No. 8,628,801 titled “Pegylated Nanoparticles” (Universidad de Navarra); US2014/0107025 titled “Ocular drug delivery system” (Jade Therapeutics, LLC); U.S. Pat. No. 6,287,588 titled “Agent delivering system comprised of microparticle and biodegradable gel with an improved releasing profile and methods of use thereof”, U.S. Pat. No. 6,589,549 titled “Bioactive agent delivering system comprised of microparticles within a biodegradable to improve release profiles” (Macromed, Inc.); U.S. Pat. Nos. 6,007,845 and 5,578,325 titled “Nanoparticles and microparticles of non-linear hydrophilichydrophobic multiblock copolymers” (Massachusetts Institute of Technology); US 2004/0234611, US 2008/0305172, US 2012/0269894, and US20130122064 titled “Ophthalmic depot formulations for periocular or subconjunctival administration (Novartis Ag); U.S. Pat. No. 6,413,539 titled “Block polymer” (Poly-Med, Inc.); US 2007/0071756 titled “Delivery of an agent to ameliorate inflammation” (Peyman); US 20080166411 titled “Injectable Depot Formulations And Methods For Providing Sustained Release Of Poorly Soluble Drugs Comprising Nanoparticles” (Pfizer, Inc.); U.S. Pat. No. 6,706,289 titled “Methods and compositions for enhanced delivery of bioactive molecules” (PR Pharmaceuticals, Inc.); and U.S. Pat. No. 8,663,674 titled “Microparticle containing matrices for drug delivery” (Surmodics).

### Uses of Active Compounds for Treatment of Selected Disorders

In one aspect, an effective amount of an active compound or its salt or composition as described herein is used to treat a medical disorder which is an inflammatory or immune condition, a disorder mediated by the complement cascade (including a dysfunctional cascade) including a complement-related disorder or alternative complement pathway-related disorder, a disorder or abnormality of a cell that adversely affects the ability of the cell to engage in or respond to normal complement activity, or an undesired complement-mediated response to a medical treatment, such as surgery or other medical procedure or a pharmaceutical or biopharmaceutical drug administration, a blood transfusion, or other allogenic tissue or fluid administration.

A complement-mediated disease or disorder is a disease or disorder in which the amount or activity of complement is such as to cause disease or disorder in an individual.

In some embodiments, the complement-mediated disease or disorder is selected from the group consisting of autoimmune disease, cancer, hematological disease, infectious disease, inflammatory disease, ischemia-reperfusion injury, neurodegenerative disease, neurodegenerative disorder, ocular disease, renal disease, transplant rejection, vascular disease, and vasculitis disease.

In some embodiments, the complement-mediated disease or disorder is an autoimmune disease. In some embodiments, the complement-mediated disease or disorder is cancer.

In some embodiments, the complement-mediated disease or disorder is an infectious disease.

In some embodiments, the complement-mediated disease or disorder is an inflammatory disease.

In some embodiments, the complement-mediated disease or disorder is a hematological disease.

In some embodiments, the complement-mediated disease or disorder is an ischemia-reperfusion injury.

In some embodiments, the complement-mediated disease or disorder is ocular disease. In some embodiments, the complement-mediated disease or disorder is a renal disease.

In some embodiments, the complement-mediated disease or disorder is transplant rejection.

In some embodiments, the complement-mediated disease or disorder is antibody-mediated transplant rejection.

In some embodiments, the complement-mediated disease or disorder is a vascular disease.

In some embodiments, the complement-mediated disease or disorder is a vasculitis disorder.

In some embodiments, the complement-mediated disease or disorder is a neurodegenerative disease or disorder.

In some embodiments, the complement-mediated disease is a neurodegenerative disease.

In some embodiments, the complement-mediated disorder is a neurodegenerative disorder. In some embodiments, the complement-mediated disease or disorder is a tauopathy.

In certain aspects, an effective amount of an active compound described herein, or it pharmaceutically acceptable salt, is used to treat a medical disorder of the central nervous system (CNS) or peripheral nervous system disorders involving complement activation. In embodiments, the CNS disorder is an acquired brain or spinal cord injury, including, but not limited to ischaemic-reperfusion injury or stroke, traumatic brain injury (TBI) and spinal cord injury (SCI).

In embodiments, the disorder is a neurodegeneration disorder. In embodiments, the disorder is a neuroinflammation disorder.

In certain aspects, an active compound described herein, or a pharmaceutically acceptable salt thereof, is used to treat Alzheimer's disease (AD). AD is characterized by two hallmark pathologies; amyloid-β (Aβ) plaques and neurofibrillary tangles comprising hyperphosphorylated tau. Recent studies have implicated complement in AD pathogenesis, including genome-wide association studies identifying single nucleotide polymorphisms (SNPs) associated with risk of late-onset AD in genes encoding complement proteins Clusterin (CLU) and CR1 (CR1). See Carpanini et al., Therapeutic Inhibition of the Complement System in Diseases of the Central Nervous System, Front. Immunol., 4 Mar. 2019. Biomarker studies have also identified complement proteins and activation products in plasma and/or CSF that distinguish AD from controls and predict risk of progression to AD. (Id.)

In certain aspects, an effective amount of active compound described herein, or a pharmaceutically acceptable salt thereof, is used to treat certain forms of frontotemporal dementia including, but not limited to, Pick's disease, sporadic Frontotemporal dementia and Frontotemporal dementia with Parkinsonism linked to chromosome 17, Progressive supranuclear palsy (PSP), Corticobasal degeneration (CBD), and Subacute sclerosing panencephalitis.

In certain aspects, an effective amount of active compound described herein, or a pharmaceutically acceptable salt thereof, is used to treat multiple sclerosis (MS). Multiple sclerosis (MS) is the most common cause of neurological disability in young adults in northern European-Caucasian populations, with an approximate lifetime risk of one in 400. C3 has been shown to be deposited in the brains of MS patients. T-cell clone (TCC) has been shown to be in association with capillary endothelial cells, predominantly within plaques and adjacent white matter. Localization of C activation to areas of active myelin destruction has also been shown, with TCC deposited exclusively in such areas. C3d has been shown to be deposited in association with short segments of disrupted myelin in plaques with low-grade active demyelination and provides evidence for a C contribution to disease progression as well as acute inflammation. See Ingram et al., Complement in multiple sclerosis: its role in disease and potential as a biomarker. Clin Exp Immunol. 2009 February; 155(2):128-39.

In certain aspects, an effective amount of an active compound described herein, or a pharmaceutically acceptable salt thereof, is used to treat neuromyelitis optica (NMO). Neuromyelitis optica (NMO) is an inflammatory demyelinating disease affecting predominantly the optic nerves and spinal cord. Traditionally seen as a variant of MS, it has been redefined recently according to new criteria using a combination of phenotypic subtyping along with a newly developed biomarker of disease, NMO-immunoglobulin G (IgG) (reported sensitivity of 58-76% and specificity of 85-99% for NMO). NMO patients have higher levels of C3a and anti-C1q antibodies than healthy controls. C3a levels correlated with disease activity, neurological disability and aquaporin-4 IgG. Nytrova et al., Complement activation in patients with neuromyelitis optica. J Neuroimmunol. 2014 Sep. 15; 274(1-2):185-91.

In certain aspects, an effective amount of an active compound as described herein, or a pharmaceutically acceptable salt thereof, is used to treat amyotrophic lateral sclerosis (ALS). ALS is caused by progressive loss of upper and lower (a) motor neurons resulting in denervation of neuromuscular junctions in the peripheral nervous system, progressive muscle weakness, atrophy, spasticity, respiratory failure, and ultimately paralysis and death. Recent studies have shown increased C1q protein in motor cortex and spinal cord of ALS post-mortem tissue; C3 activation fragments and TCC in areas of pathology; C4d and TCC staining of degenerating neurons and glia in ALS motor cortex and spinal cord, and C5aR1 upregulation in areas of pathology. C3d and C4d have been found on oligodendroglia and degenerating neurites, surrounded by CR4-positive microglia, in spinal cord and motor cortex, and C1q, C3, and TCC have been shown to be present on motor end-plates in intercostal muscles in ALS donors even early in the disease process. See Carpanini et al., Therapeutic Inhibition of the Complement System in Diseases of the Central Nervous System, Front. Immunol., 4 Mar. 2019.

In certain aspects, an effective amount of an active compound described herein, or a pharmaceutically acceptable salt thereof, is used to treat Parkinson's disease (PD). PD is characterized by loss of dopaminergic neurons in the substantia nigra and deposits of the protein α-synuclein that form the pathological hallmarks of the disease, Lewy bodies. Patients present with resting tremor, bradykinesia, and rigidity. Complement activation has been associated with α-synuclein and Lewy bodies in Parkinson's disease; in vitro studies have demonstrated that the disease-associated splice variant α-synuclein 112, but not the full-length protein, cause activation of complement. In vivo, C3d, C4d, C7 and C9 localization in Lewy bodies has been reported. More recently, deposition of iC3b and C9 in Lewy bodies and melanized neurons has been reported, and iC3b immunoreactivity has been shown to be increased with normal ageing and was further elevated in PD vs. age-matched controls. Furthermore, correlation between the ratios of C₃/Aβ42 or FH/Aβ42 in CSF and severity of Parkinson's disease motor and cognitive symptoms has been shown. See Carpanini et al., Therapeutic Inhibition of the Complement System in Diseases of the Central Nervous System, Front. Immunol., 4 Mar. 2019. In some embodiments, the subject to be treated suffers from Parkinson's Disease with dementia (PDD).

In certain aspects, an effective amount of an active compound described herein, or a pharmaceutically acceptable salt thereof, is used to treat Huntington's disease (HD). HD is an autosomal dominant, inherited neurodegenerative disease characterized by progressive motor symptoms, psychiatric disturbances, and dementia. It is caused by expansion of a three-base-pair (CAG) repeat (39-121 repeats vs. normal range 8-39 repeats) in exon 1 of the HTT gene that translates into a polyglutamine tract at the N-terminus of the protein. This results in a polyglutamine length-dependent misfolding and accumulation of huntingtin protein in the striatum and cortex (layers 3, 5, and 6) followed by neuronal loss in these areas which spreads to the hippocampus. It has been shown that neurons, astrocytes, and myelin sheaths in the HD caudate and striatum were immunoreactive for C1q, C4, C3 and neo-epitopes in iC3b and TCC. Expression of mRNA encoding early complement components C1q (c-chain), C1r, C3, and C4, complement regulators C1INH, Clusterin, MCP, DAF and CD59, and complement receptors C3a and C5a, have been shown to be upregulated in the HD striatum, see Carpanini et al., Therapeutic Inhibition of the Complement System in Diseases of the Central Nervous System, Front. Immunol., 4 Mar. 2019.

In certain aspects, an effective amount of an active compound described herein, or a pharmaceutically acceptable salt thereof, is used to treat argyrophilic grain dementia, British type amyloid angiopathy, cerebral amyloid angiopathy, Creutzfeldt-Jakob disease, dementia pugilistica, diffuse neurofibrillary tangles with calcification, Down's syndrome, frontotemporal lobar degeneration, Gerstmann-Straussler-Scheinker disease, Hallervorden-Spatz disease, inclusion body myositis, multiple system atrophy (MSA), myotonic dystrophy, Niemann-Pick disease type C, non-Guamanian motor neuron disease with neurofibrillary tangles, postencephalitic parkinsonism, prion protein cerebral amyloid angiopathy, progressive subcortical gliosis, progressive supranuclear palsy, subacute sclerosing panencephalitis, Tangle only dementia, multi-infarct dementia, ischemic stroke, chronic traumatic encephalopathy (CTE), traumatic brain injury (TBI), and stroke.

In certain aspects, an effective amount of an active compound described herein, or a pharmaceutically acceptable salt thereof, is used to treat a hereditary motor and sensory neuropathy (HMSN).

In some embodiments, the hereditary and sensory neuropathy is Charcot-Marie-Tooth (CMT) disease.

In some embodiments, the HSMN is Charcot-Marie-Tooth disease type 1A or type 1B.

In some embodiments, the HSMN is Charcot-Marie-Tooth disease type 2.

In some embodiments, the HSMN is Dejerine-Sottas disease (Charcot-Marie-Tooth type 3).

In some embodiments, the HSMN is Refsum disease.

In some embodiments, the HSMN is Charcot-Marie-Tooth with pyramidal features. In some embodiments, the HSMN is Charcot-Marie-Tooth type 6. In some embodiments, the HSMN is HMSN+retinitis pigmentosa.

In some embodiments, an effective amount of an active compound described herein, or a pharmaceutically acceptable salt thereof, is used to treat Churg-Strauss syndrome.

In some embodiments, an effective amount of an active compound described herein, or a pharmaceutically acceptable salt thereof, is used to treat a peripheral artery disease (PAD).

In certain aspects, an effective amount of an active compound described herein, or a pharmaceutically acceptable salt thereof, is used to treat myasthenia gravis with CNS involvement.

In certain aspects, an effective amount of an active compound described herein, or a pharmaceutically acceptable salt thereof, is used to treat dementia with Lewy bodies.

In certain aspects, an effective amount of an active compound described herein, or a pharmaceutically acceptable salt thereof, is used to treat an individual suffering from prion disease.

In certain aspects, an effective amount of an active compound described herein, or a pharmaceutically acceptable salt thereof, is used to treat Behcet's Disease.

In certain aspects, an effective amount of an active compound described herein, or a pharmaceutically acceptable salt thereof, is used to treat congenital myasthenia.

In certain aspects, an effective amount of an active compound described herein, or a pharmaceutically acceptable salt thereof, is used to treat subacute sclerosing panencephalitis (SSPE).

In certain aspects, an effective amount of an active compound described herein, or a pharmaceutically acceptable salt thereof, is used to treat Guillain-Barre syndrome.

In certain aspects, the CNS disorder to be treated is a demyelinating disease, including, but not limited to, demyelinating myelinoclastic diseases and demyelinating leukostrophic disease.

In certain aspects, the disorder to be treated is a demyelinating myelonoclastic disease including, but not limited to, multiple sclerosis, neuromyelitis optica, neuromyelitis optica spectrum of disorders (NMOSD), idiopathic inflammatory demyelinating diseases (IIDD), anti-NMDA receptor encephalitis, acute disseminated encephalomyelitis, anti-MOG autoimmune encephalomyelitis, chronic relapsing inflammatory optic neuritis (CRION), acute disseminated encephalomyelitis (ADEM), immune-mediated encephalomyelitis, progressive multifocal leukoencephalopathy (PML); McDonalds-positive multiple sclerosis, acute hemorrhagic leukoencephalitis, Rasmussen's Encephalitis, Marburg multiple sclerosis, pseudotumefactive and tumefactive multiple sclerosis, Balo concentric sclerosis, diffuse myelinoclastic sclerosis, solitary sclerosis, multiple sclerosis with cavitary lesions, myelocortical multiple sclerosis (MCMS), atypical optic-spinal multiple sclerosis, pure spinal multiple sclerosis, HLA DRB3*02:02 multiple sclerosis, autoimmune GFAP astrocytopathy, Chronic inflammatory demyelinating polyneuropathy (CIDP), Guillain-Barre syndrome, progressive inflammatory neuropathy, Lewis-Sumner Syndrome, combined central and peripheral demyelination (CCPD), Bickerstaff brainstem encephalitis, Fisher syndrome, trigeminal neuralgia, NMDAR anti-NMDA receptor encephalitis, primary progressive MS (PPMS), OPA1 variant multiple sclerosis, KIR4.1 multiple sclerosis, aquaporine-related multiple sclerosis, chronic cerebrospinal venous insufficiency (CCSVI or CCVI), diffuse sclerosis, and Schilder's disease.

In certain aspects, the disorder to be treated is a demyelinating leukostrophic disease including, but not limited to, myelitis, central pontine myelinolysis (CPM), extrapontine myelinolysis, tabes dorsalis, progressive multifocal leukoencephalopathy, leukoencephalopathy with vanishing white matter, leukoencephalopathy with neuroaxonal spheroids, reversible posterior leukoencephalopathy syndrome, megalencephalic leukoencephalopathy with subcortical cysts, megalencephalic leukoencephalopathy with subcortical cysts 1, hypertensive leukoencephalopathy, Metachromatic leukodystrophy, Krabbe disease, Canavan disease, X-linked adrenoleukodystrophy, Alexander disease, cerebrotendineous xanthomatosis, Pelizaeus-Merzbacher disease, and Refsum disease.

In some embodiments, an effective amount of an active compound described herein, or a pharmaceutically acceptable salt thereof, is used to treat Buerger's disease, also known as thromboangiitis obliterans.

In some embodiments, an effective amount of an active compound described herein, or a pharmaceutically acceptable salt thereof, is used to treat giant cell arteritis.

In some embodiments, an effective amount of an active compound described herein, or a pharmaceutically acceptable salt thereof, is used to treat Raynaud's disease.

In certain aspects, the disorder to be treated is a demyelinating disease of the peripheral nervous system, including, but not limited to, Guillain-Barre syndrome and its chronic counterpart, chronic inflammatory demyelinating polyneuropathy, anti-MAG peripheral neuropathy, Charcot-Marie-Tooth disease and its counterpart Hereditary neuropathy with liability to pressure palsy, Copper deficiency-associated conditions (peripheral neuropathy, myelopathy, and rarely optic neuropathy), and progressive inflammatory neuropathy.

In certain aspects, the disorder to be treated is a neurological inflammatory disorder. In certain embodiments, the disorder to be treated includes, but is not limited to, cranial arteritis; giant cell arteritis; Holmes-Adie syndrome; inclusion body myositis (IBM); meningitis; neurologic paraneoplastic syndrome including, but not limited to, Lambert-Eaton myasthenic syndrome, stiff-person syndrome, encephalomyelitis (inflammation of the brain and spinal cord), myasthenia gravis, cerebellar degeneration, limbic and/or brainstem encephalitis, neuromyotonia, and opsoclonus (involving eye movement) and sensory neuropathy; polymyositis; transverse myelitis; vasculitis including temporal arteritis; arachnoiditis; Kinsbourne syndrome or opsoclonus myoclonus syndrome (OMS); or Saint Vitus Dance or sydenham chorea (SD) disease.

In some embodiments, an effective amount of an active compound described herein, or a pharmaceutically acceptable salt thereof, is used to treat transverse myelitis.

In certain aspects, the disorder to be treated is a peripheral neuropathy. In some embodiments, the peripheral neuropathy is a mononeuropathy. In some embodiments, the neuropathy is a polyneuropathy. In some embodiments, the polyneuropathy is distal axonopathy, diabetic neuropathy, a demyelinating polyneuropathy, small fiber peripheral neuropathy, mononeuritis multiplex, polyneuritis multiplex, autonomic neuropathy, or neuritis.

In some embodiments, an effective amount of an active compound described herein, or a pharmaceutically acceptable salt thereof, is used to treat an autoimmune vascular disease. In some embodiments, the autoimmune vascular disease is vasculitis. In some embodiments, the vasculitis includes, but is not limited to, autoimmune inflammatory vasculitis, Cutaneous small-vessel vasculitis, Granulomatosis with polyangiitis, Eosinophilic granulomatosis with polyangiitis, Behçet's disease, Kawasaki disease, Buerger's disease, and “Limited” granulomatosis with polyangiitis vasculitis.

In some embodiments, an active compound or its salt or composition as described herein is used to treat an arteritis. Is some embodiments, the arteritis includes, but is not limited to, giant cell arteritis, Takayasu arteritis, temporal arteritis, and polyarteritis nodosa.

In some embodiments, a method for the treatment of a glomerulonephritis is provided. In some embodiment, the glomerulonephritis is membranoproliferative glomerulonephritis (MPGN). In some embodiments, the MPGN is MPGN Type I. In some embodiments, the MPGN is MPGN Type II. In some embodiments, the MPGN is MPGN Type III. In some embodiments, the MPGN is C3 glomerulonephritis (C3G). In some embodiments, the MPGN is dense deposit disease (DDD). In some embodiments, the MPGN is a C4 deposition disorder.

In some embodiments, the glomerulonephritis is IC-MPGN. In some embodiments, the glomerulonephritis is a membraneous glomerulonephritis. In some embodiments, the glomerulonephritis is IgA nephropathy. In some embodiments, the glomerulonephritis is Post-infectious glomerulonephritis. In some embodiments, the glomerulonephritis is a rapidly progressive glomerulonephritis, for example Type I (Goodpasture syndrome), Type II, or Type III rapidly progressive glomerulonephritis.

In some embodiments, a method for the treatment of paroxysmal nocturnal hemoglobinuria (PNH) is provided that includes the administration of an effective amount of a compound to a host of Formula I, II, III, IV, V, VI, VII, VIII, IX, X, XI, XII, XIII, XIV, XV, XVI, XVII, XVIII, XIX, or XX or a pharmaceutically acceptable salt, prodrug, N-oxide, or isolated isomer thereof, optionally in a pharmaceutically acceptable composition.

In some embodiments, a method for the treatment of hereditary angioedema (HAE) is provided that includes the administration of an effective amount of a compound to a host of Formula I, II, III, IV, V, VI, VII, VIII, IX, X, XI, XII, XIII, XIV, XV, XVI, XVII, XVIII, XIX, or XX, or a pharmaceutically acceptable salt, prodrug, N-oxide, or isolated isomer thereof, optionally in a pharmaceutically acceptable composition. Mutations in the SERPINGI gene cause hereditary angioedema type I and type II. Hereditary angioedema is a disorder characterized by recurrent episodes of severe swelling (angioedema). The most common areas of the body to develop swelling are the limbs, face, intestinal tract, and airway. The SERPINGI gene provides instructions for making the C1 inhibitor protein, which is important for controlling inflammation. C1 inhibitor blocks the activity of certain proteins that promote inflammation. Mutations that cause hereditary angioedema type I lead to reduced levels of C1 inhibitor in the blood, while mutations that cause type II result in the production of a C1 inhibitor that functions abnormally. Without the proper levels of functional C1 inhibitor, excessive amounts of a protein fragment (peptide) called bradykinin are generated. Bradykinin promotes inflammation by increasing the leakage of fluid through the walls of blood vessels into body tissues. Excessive accumulation of fluids in body tissues causes the episodes of swelling seen in individuals with hereditary angioedema type I and type II.

In some embodiments, a method for the treatment of cold agglutinin disease (CAD) is provided that includes the administration of an effective amount of a compound to a host of Formula I, II, III, IV, V, VI, VII, VIII, IX, X, XI, XII, XIII, XIV, XV, XVI, XVII, XVIII, XIX, or XX, or a pharmaceutically acceptable salt, prodrug, N-oxide, or isolated isomer thereof, optionally in a pharmaceutically acceptable composition. CAD is a rare autoimmune hemolytic condition with potentially serious acute and chronic consequences that are driven by C1 activation of the classical complement pathway.

In some embodiments, a method for the treatment of atypical hemolytic uremic syndrome (aHUS) is provided that includes the administration of an effective amount of a compound to a host of Formula I, II, III, IV, V, VI, VII, VIII, IX, X, XI, XII, XIII, XIV, XV, XVI, XVII, XVIII, XIX, or XX, or a pharmaceutically acceptable salt, prodrug, N-oxide, or isolated isomer thereof, optionally in a pharmaceutically acceptable composition. Atypical hemolytic-uremic syndrome is a disease that primarily affects kidney function. Atypical hemolytic uremic syndrome, which can occur at any age, causes abnormal blood clots (thrombi) to form in small blood vessels in the kidneys. These clots can cause serious medical problems if they restrict or block blood flow. Atypical hemolytic-uremic syndrome is characterized by three major features related to abnormal clotting: hemolytic anemia, thrombocytopenia, and kidney failure.

In another embodiment, a method for the treatment of wet or dry age-related macular degeneration (AMD) in a host is provided that includes the administration of an effective amount of a compound of Formula I, II, III, IV, V, VI, VII, VIII, IX, X, XI, XII, XIII, XIV, XV, XVI, XVII, XVIII, XIX, or XX, or a pharmaceutically acceptable salt, prodrug, N-oxide, or isolated isomer thereof, optionally in a pharmaceutically acceptable composition. In another embodiment, a method for the treatment of rheumatoid arthritis in a host is provided that includes the administration of an effective amount of a compound of Formula I, II, III, IV, V, VI, VII, VIII, IX, X, XI, XII, XIII, XIV, XV, XVI, XVII, XVIII, XIX, or XX, or a pharmaceutically acceptable salt, prodrug, N-oxide, or isolated isomer thereof, optionally in a pharmaceutically acceptable composition.

In another embodiment, a method for the treatment of multiple sclerosis in a host is provided that includes the administration of an effective amount of a compound of Formula I, II, III, IV, V, VI, VII, VIII, IX, X, XI, XII, XIII, XIV, XV, XVI, XVII, XVIII, XIX, or XX, or a pharmaceutically acceptable salt, prodrug, N-oxide, or isolated isomer thereof, optionally in a pharmaceutically acceptable composition.

The active compounds, or pharmaceutically acceptable salt, prodrug, N-oxide, or isolated isomer thereof, optionally in a pharmaceutically acceptable composition, as disclosed herein, are also useful for administration in combination (in the same or a different dosage form) or alternation with a second pharmaceutical agent for use in ameliorating or reducing a side effect of the second pharmaceutical agent.

For example, in some embodiments, the active compound may be used in combination with an adoptive cell-transfer therapy to reduce an inflammatory response associated with such therapy, for example, a cytokine mediated response such as cytokine response syndrome.

In some embodiments, the adoptive cell-transfer therapy is a chimeric antigen receptor T-Cell (CAR T) or a dendritic cell used to treat a hematologic or solid tumor, for example, a B-cell related hematologic cancer.

In some embodiments, the hematologic or solid tumor is acute lymphoblastic leukemia (ALL), acute myeloid leukemia (AML), non-Hodgkin's lymphoma, chronic lymphocytic leukemia (CLL), pancreatic cancer, glioblastoma, or a cancer that expresses CD19.

In some embodiments, the adoptive cell-transfer therapy is a non-engineered T-cell therapy, wherein the T-cells have been activated and/or expanded to one or more viral or tumor antigens. In some embodiments, the associated inflammatory response is a cytokine mediated response.

In some embodiments, the second pharmaceutical agent is a cell that has been transformed to express a protein, wherein the protein in the host is mutated or otherwise has impaired function. In some embodiments, the transformed cell includes a CRISPR gene.

Another embodiment is provided that includes the administration of an effective amount of an active compound, or a pharmaceutically acceptable salt, prodrug, N-oxide, or isolated isomer thereof, optionally in a pharmaceutically acceptable composition to a host to treat an ocular, pulmonary, gastrointestinal, or other disorder.

Any of the compounds described herein (e.g. a compound of any one of Formulas I, II, III, IV, V, VI, VII, VIII, IX, X, XI, XII, XIII, XIV, XV, XVI, XVII, XVIII, XIX, or XX) can be administered to the eye in any desired form of administration, including via intravitreal, intrastromal, intracameral, sub-tenon, sub-retinal, retro-bulbar, peribulbar, suprachorodial, choroidal, subchoroidal, conjunctival, subconjunctival, episcleral, posterior juxtascleral, scleral, circumcorneal, and tear duct injections, or through a mucus, mucin, or a mucosal barrier, in an immediate or controlled release fashion. In certain embodiments, the active compound includes a lipophilic group, such as a lipophilic acyl group, which is delivered to the eye in a polymeric drug delivery system such as polylactic acid, polylactide-co-glycolide, polyglycolide or other erodible polymer, or a combination thereof, or in another type of lipophilic material for ocular delivery. In some embodiments, the lipophilic active molecule is more soluble in the polymeric or other form of delivery system than in ocular fluid.

In other embodiments of the disclosure, an active compound provided herein can be used to treat or prevent a disorder in a host mediated by complement. As examples, the disclosure includes methods to treat or prevent complement associated disorders that are induced by antibody-antigen interactions, a component of an immune or autoimmune disorder or by ischemic injury. The disclosure also provides methods to decrease inflammation or an immune response, including an autoimmune response, where mediated or affected by the classical complement pathway.

In some embodiments, the disorder is selected from fatty liver and conditions stemming from fatty liver, such as nonalcoholic steatohepatitis (NASH), liver inflammation, cirrhosis and liver failure. In some embodiments of the present disclosure, a method is provided for treating fatty liver disease in a host by administering an effective amount of an active compound or its salt or composition as described herein.

In another embodiment, an active compound or its salt or composition as described herein is used to modulate an immune response prior to or during surgery or other medical procedure. One non-limiting example is use in connection with acute or chronic graft versus host disease, which is a common complication as a result of organ transplantation, allogeneic tissue transplant, and can also occur as a result of a blood transfusion.

In some embodiments, the present disclosure provides a method of treating or preventing dermatomyositis by administering to a subject in need thereof an effective amount of an active compound or its salt or composition as described herein.

In some embodiments, the present disclosure provides a method of treating or preventing amyotrophic lateral sclerosis by administering to a subject in need thereof an effective amount of an active compound or its salt or composition as described herein.

In some embodiments, the present disclosure provides a method of treating or preventing abdominal aortic aneurysm, hemodialysis complications, hemolytic anemia, or hemodialysis by administering to a subject in need thereof an effective amount of an active compound or its salt or composition as described herein.

In another embodiment, a method is provided for the treatment or prevention of cytokine or inflammatory reactions in response to the administration of pharmaceutical or biotherapeutic (e.g., CAR T-cell therapy or monoclonal antibody therapy) in a host by administering an effective amount of an active compound or its salt or composition as described herein. Various types of cytokine or inflammatory reactions may occur in response to a number of factors, such as the administrations of biotherapeutics.

In some embodiments, the cytokine or inflammatory reaction is cytokine release syndrome. In some embodiments, the cytokine or inflammatory reaction is tumor lysis syndrome (which also leads to cytokine release). Symptoms of cytokine release syndrome range from fever, headache, and skin rashes to bronchospasm, hypotension and even cardiac arrest. Severe cytokine release syndrome is described as a cytokine storm, and can be fatal.

Fatal cytokine storms have been observed in response to infusion with several monoclonal antibody therapeutics. See, Abramowicz D, et al. “Release of tumor necrosis factor, interleukin-2, and gamma-interferon in serum after injection of OKT3 monoclonal antibody in kidney transplant recipients” Transplantation (1989) 47(4):606-8; Chatenoud L, et al. “In vivo cell activation following OKT3 administration. Systemic cytokine release and modulation by corticosteroids” Transplantation (1990) 49(4):697-702; and Lim L C, Koh L P, and Tan P. “Fatal cytokine release syndrome with chimeric anti-CD20 monoclonal antibody rituximab in a 71-year-old patient with chronic lymphocytic leukemia” J. Clin Oncol. (1999) 17(6):1962-3.

Also contemplated herein, is the use of an active compound or its salt or composition as described herein to mediate an adverse immune response in patients receiving bi-specific T-cell engagers (BiTE). A bi-specific T-cell engager directs T-cells to target and bind with a specific antigen on the surface of a cancer cell. For example, Blinatumomab (Amgen), a BiTE has recently been approved as a second line therapy in Philadelphia chromosome-negative relapsed or refractory acute lymphoblastic leukemia. Blinatumomab is given by continuous intravenous infusion in 4-week cycles. The use of BiTE agents has been associated with adverse immune responses, including cytokine release syndrome. The most significantly elevated cytokines in the CRS associated with ACT include IL-10, IL-6, and IFN-γ (Klinger et al., Immunopharmacologic response of patients with B-lineage acute lymphoblastic leukemia to continuous infusion of T cell-engaging CD19/CD3-bispecific BiTE antibody blinatumomab. Blood (2012) 119:6226-6233).

In another embodiment, the disorder is episcleritis, idiopathic episcleritis, anterior episcleritis, or posterior episcleritis. In some embodiments, the disorder is idiopathic anterior uveitis, HLA-B27 related uveitis, herpetic keratouveitis, Posner Schlossman syndrome, Fuch's heterochromic iridocyclitis, or cytomegalovirus anterior uveitis.

In some embodiments, the present disclosure provides a method of treating or preventing a IC-MPGN by administering to a subject in need thereof an effective amount of an active compound or its salt or composition as described herein.

In some embodiments, the present disclosure provides a method of treating or preventing a paroxysmal nocturnal hemoglobinuria (PNH) by administering to a subject in need thereof an effective amount of an active compound or its salt or composition as described herein.

In some embodiments, the present disclosure provides a method of treating or preventing a hereditary angioedema (HAE) by administering to a subject in need thereof an effective amount of an active compound or its salt or composition as described herein.

In some embodiments, the present disclosure provides a method of treating or preventing cold agglutinin disease (CAD) by administering to a subject in need thereof an effective amount of an active compound or its salt or composition as described herein.

In some embodiments, the present disclosure provides a method of treating or preventing atypical hemolytic syndrome (aHUS) by administering to a subject in need thereof an effective amount of an active compound or its salt or composition as described herein.

In some embodiments, the present disclosure provides a method of treating or preventing age-related macular degeneration (AMD) by administering to a subject in need thereof an effective amount of an active compound or its salt or composition as described herein.

In some embodiments, the present disclosure provides a method of treating or preventing rheumatoid arthritis by administering to a subject in need thereof an effective amount of an active compound or its salt or composition as described herein.

In some embodiments, the present disclosure provides a method of treating or preventing multiple sclerosis by administering to a subject in need thereof an effective amount of an active compound or its salt or composition as described herein.

In some embodiments, the present disclosure provides a method of treating or preventing myasthenia gravis by administering to a subject in need thereof an effective amount of an active compound or its salt or composition as described herein.

In some embodiments, the present disclosure provides a method of treating or preventing atypical hemolytic uremic syndrome (aHUS) by administering to a subject in need thereof an effective amount of an active compound or its salt or composition as described herein.

In yet another embodiment, the present disclosure provides a method of treating or preventing a disorder as described below by administering to a subject in need thereof an effective amount of an active compound or its salt or composition as described herein, including:
vitritis, sarcoidosis, syphilis, tuberculosis, or Lyme disease; retinal vasculitis, Eales disease, tuberculosis, syphilis, or toxoplasmosis; neuroretinitis, viral retinitis, or acute retinal necrosis; varicella zoster virus, herpes simplex virus, cytomegalovirus, Epstein-Barr virus, lichen planus, or Dengue-associated disease (e.g., hemorraghic Dengue Fever); Masquerade syndrome, contact dermatitis, trauma induced inflammation, UVB induced inflammation, eczema, granuloma annulare, or acne.

In an additional embodiment, the disorder is selected from: acute myocardial infarction, aneurysm, cardiopulmonary bypass, dilated cardiomyopathy, complement activation during cardiopulmonary bypass operations, coronary artery disease, restenosis following stent placement, or percutaneous transluminal coronary angioplasty (PTCA); antibody-mediated transplant rejection, anaphylactic shock, anaphylaxis, allogenic transplant, humoral and vascular transplant rejection, graft dysfunction, graft-versus-host disease, Graves' disease, adverse drug reactions, or chronic graft vasculopathy; allergic bronchopulmonary aspergillosis, allergic neuritis, drug allergy, radiation-induced lung injury, eosinophilic pneumonia, radiographic contrast media allergy, bronchiolitis obliterans, or interstitial pneumonia; parkinsonism-dementia complex, sporadic frontotemporal dementia, frontotemporal dementia with Parkinsonism linked to chromosome 17, frontotemporal lobar degeneration, tangle only dementia, cerebral amyloid angiopathy, cerebrovascular disorder, certain forms of frontotemporal dementia, chronic traumatic encephalopathy (CTE), Parkinson's Disease with dementia (PDD), argyrophilic grain dementia, dementia pugilistica, dementia with Lewy Bodies (DLB), or multi-infarct dementia; Creutzfeldt-Jakob disease, Huntington's disease, multifocal motor neuropathy (MMN), prion protein cerebral amyloid angiopathy, polymyositis, postencephalitic parkinsonism, subacute sclerosing panencephalitis, non-Guamanian motor neuron disease with neurofibrillary tangles, neural regeneration, and diffuse neurofibrillary tangles with calcification.

In some embodiments, the disorder is selected from: atopic dermatitis, dermatitis, dermatomyositis bullous pemphigoid, scleroderma, sclerodermatomyositis, psoriatic arthritis, pemphigus vulgaris, Discoid lupus erythematosus, cutaneous lupus, chilblain lupus erythematosus, or lupus erythematosus-lichen planus overlap syndrome; cryoglobulinemic vasculitis, mesenteric/enteric vascular disorder, peripheral vascular disorder, antineutrophil cytoplasm antibody (ANCA)-associated vasculitis (AAV), IL-2 induced vascular leakage syndrome, or immune complex vasculitis; angioedema, low platelets (HELLP) syndrome, sickle cell disease, platelet refractoriness, red cell casts, or typical or infectious hemolytic uremic syndrome (tHUS); hematuria, hemorrhagic shock, drug-induced thrombocytopenia, autoimmune hemolytic anemia (AIHA), azotemia, blood vessel and/or lymph vessel inflammation, rotational atherectomy, or delayed hemolytic transfusion reaction; British type amyloid angiopathy, Buerger's disease, bullous pemphigoid, C1q nephropathy, cancer, and catastrophic antiphospholipid syndrome.

In another embodiment, the disorder is selected from: wet (exudative) AMD, dry (non-exudative) AMD, chorioretinal degeneration, choroidal neovascularization (CNV), choroiditis, loss of RPE function, loss of vision (including loss of visual acuity or visual field), loss of vision from AMD, retinal damage in response to light exposure, retinal degeneration, retinal detachment, retinal dysfunction, retinal neovascularization (RNV), retinopathy of prematurity, pathological myopia, or RPE degeneration; pseudophakic bullous keratopathy, symptomatic macular degeneration related disorder, optic nerve degeneration, photoreceptor degeneration, cone degeneration, loss of photoreceptor cells, pars planitis, scleritis, proliferative vitreoretinopathy, or formation of ocular drusen; chronic urticaria, Churg-Strauss syndrome, cold agglutinin disease (CAD), corticobasal degeneration (CBD), cryoglobulinemia, cyclitis, damage of the Bruch's membrane, Degos disease, diabetic angiopathy, elevated liver enzymes, endotoxemia, epidermolysis bullosa, or epidermolysis bullosa acquisita; essential mixed cryoglobulinemia, excessive blood urea nitrogen-BUN, focal segmental glomerulosclerosis, Gerstmann-Straussler-Scheinker disease, giant cell arteritis, gout, Hallervorden-Spatz disease, Hashimoto's thyroiditis, Henoch-Schonlein purpura nephritis, or abnormal urinary sediments; hepatitis, hepatitis A, hepatitis B, hepatitis C or human immunodeficiency virus (HIV), a viral infection more generally, for example selected from Flaviviridae, Retroviruses, Coronaviridae, Poxviridae, Adenoviridae, Herpesviridae, Caliciviridae, Reoviridae, Picornaviridae, Togaviridae, Orthomyxoviridae, Rhabdoviridae, or Hepadnaviridae; Neisseria meningitidis, shiga toxin E. coli-related hemolytic uremic syndrome (STEC-HUS), hemolytic uremic syndrome (HUS); Streptococcus, and poststreptococcal glomerulonephritis.

In a further embodiment, the disorder is selected from: hyperlipidemia, hypertension, hypoalbuminemia, hypobolemic shock, hypocomplementemic urticarial vasculitis syndrome, hypophosphastasis, hypovolemic shock, idiopathic pneumonia syndrome, or idiopathic pulmonary fibrosis; inclusion body myositis, intestinal ischemia, iridocyclitis, iritis, juvenile chronic arthritis, Kawasaki's disease (arteritis), or lipiduria; membranoproliferative glomerulonephritis (MPGN) I, microscopic polyangiitis, mixed cryoglobulinemia, molybdenum cofactor deficiency (MoCD) type A, pancreatitis, panniculitis, Pick's disease, polyarteritis nodosa (PAN), progressive subcortical gliosis, proteinuria, reduced glomerular filtration rate (GFR), or renovascular disorder; multiple organ failure, multiple system atrophy (MSA), myotonic dystrophy, Niemann-Pick disease type C, chronic demyelinating diseases, or progressive supranuclear palsy; spinal cord injury, spinal muscular atrophy, spondyloarthropathies, Reiter's syndrome, spontaneous fetal loss, recurrent fetal loss, pre-eclampsia, synucleinopathy, Takayasu's arteritis, post-partum thryoiditis, thyroiditis, Type I cryoglobulinemia, Type II mixed cryoglobulinemia, Type III mixed cryoglobulinemia, ulcerative colitis, uremia, urticaria, venous gas embolus (VGE), or Wegener's granulomatosis; von Hippel-Lindau disease, histoplasmosis of the eye, hard drusen, soft drusen, pigment clumping, and photoreceptor and/or retinal pigmented epithelia (RPE) loss.

In some embodiments, an active compound or its salt or composition as described herein is useful for treating or preventing a disorder selected from autoimmune oophoritis, endometriosis, autoimmune orchitis, Ord's thyroiditis, autoimmune enteropathy, coeliac disease, Hashimoto's encephalopathy, antiphospholipid syndrome (APLS) (Hughes syndrome), aplastic anemia, autoimmune lymphoproliferative syndrome (Canale-Smith syndrome), autoimmune neutropenia, Evans syndrome, pernicious anemia, pure red cell aplasia, thrombocytopenia, adipose dolorosa (Dercum's disease), adult onset Still's disease, ankylosing spondylitis, CREST syndrome, drug-induced lupus, eosinophilic fasciitis (Shulman's syndrome), Felty syndrome, IgG4-related disease, mixed connective tissue disease (MCTD), palindromic rheumatism (Hench-Rosenberg syndrome), Parry-Romberg syndrome, Parsonage-Turner syndrome, relapsing polychondritis (Meyenburg-Altherr-Uehlinger syndrome), retroperitonial fibrosis, rheumatic fever, Schnitzler syndrome, fibromyalgia, neuromyotonia (Isaac's disease), paraneoplastic degeneration, autoimmune inner ear disease, Meniere's disease, interstitial cystitis, autoimmune pancreatitis, zika virus-related disorders, chikungunya virus-related disorders, subacute bacterial endocarditis (SBE), IgA nephropathy, IgA vasculitis, polymyalgia rheumatic, rheumatoid vasculitis, alopecia areata, autoimmune progesterone dermatitis, dermatitis herpetiformis, erythema nodosum, gestational pemphigoid, hidradenitis suppurativa, lichen sclerosus, linear IgA disease (LAD), morphea, myositis, pityriasis lichenoides et varioliformis acuta, vitiligo post-myocardial infarction syndrome (Dressier's syndrome), post-pericardiotomy syndrome, autoimmune retinopathy, Cogan syndrome, Graves opthalmopathy, ligneous conjunctivitis, Mooren's ulcer, opsoclonus myoclonus syndrome, optic neuritis, retinocochleocerebral vasculopathy (Susac's syndrome), sympathetic opthalmia, Tolosa-Hunt syndrome, interstitial lung disease, antisynthetase syndrome, Addison's disease, autoimmune polyendocrine syndrome (APS) type I, autoimmune polyendocrine syndrome (APS) type II, autoimmune polyendocrine syndrome (APS) type III, disseminated sclerosis (multiple sclerosis, pattern II), rapidly progressing glomerulonephritis (RPGN), juvenile rheumatoid arthritis, enthesitis-related arthritis, reactive arthritis (Reiter's syndrome), autoimmune hepatitis or lupoid hepatitis, primary biliary cirrhosis (PBS), primary sclerosing cholangitis, microscopic colitis, latent lupus (undifferentiated connective tissue disease (UCTD)), acute disseminated encephalomyelitis (ADEM), acute motor axonal neuropathy, anti-n-methyl-D-aspartate receptor encephalitis, Balo concentric sclerosis (Schilders disease), Bickerstaff's encephalitis, chronic inflammatory demyelinating polyneuropathy, idiopathic inflammatory demyelinating disease, Lambert-Eaton mysathenic syndrome, Oshtoran syndrome, pediatric autoimmune neuropsychiatric disorder associated with streptococcus (PANDAS), progressive inflammatory neuropathy, restless leg syndrome, stiff person syndrome, Sydenhem syndrome, transverse myelitis, lupus vasculitis, leukocytoclastic vasculitis, Microscopic Polyangiitis, polymyositis, and ischemic-reperfusion injury of the eye.

Examples of eye disorders that may be treated according to the compositions and methods disclosed herein include amoebic keratitis, fungal keratitis, bacterial keratitis, viral keratitis, onchorcercal keratitis, bacterial keratoconjunctivitis, viral keratoconjunctivitis, corneal dystrophic diseases, Fuchs' endothelial dystrophy, Sjogren's syndrome, Stevens-Johnson syndrome, autoimmune dry eye diseases, environmental dry eye diseases, corneal neovascularization diseases, post-corneal transplant rejection prophylaxis and treatment, autoimmune uveitis, infectious uveitis, posterior uveitis (including toxoplasmosis), pan-uveitis, an inflammatory disease of the vitreous or retina, endophthalmitis prophylaxis and treatment, macular edema, macular degeneration, age related macular degeneration, proliferative and non-proliferative diabetic retinopathy, hypertensive retinopathy, an autoimmune disease of the retina, primary and metastatic intraocular melanoma, other intraocular metastatic tumors, open angle glaucoma, closed angle glaucoma, pigmentary glaucoma, and combinations thereof.

In a further embodiment, the disorder is selected from glaucoma, diabetic retinopathy, blistering cutaneous diseases (including bullous pemphigoid, pemphigus, and epidermolysis bullosa), ocular cicatrical pemphigoid, uveitis, adult macular degeneration, diabetic retinopa retinitis pigmentosa, macular edema, diabetic macular edema, Behcet's uveitis, multifocal choroiditis, Vogt-Koyangi-Harada syndrome, imtermediate uveitis, birdshot retino-chorioditis, sympathetic ophthalmia, ocular dicatricial pemphigoid, ocular pemphigus, nonartertic ischemic optic neuropathy, postoperative inflammation, and retinal vein occlusion, and central retinal vein occulusion (CVRO).

In some embodiments, complement mediated diseases include ophthalmic diseases (including early or neovascular age-related macular degeneration and geographic atrophy), autoimmune diseases (including arthritis, rheumatoid arthritis), respiratory diseases, and cardiovascular diseases. In other embodiments, the compounds of the disclosure are suitable for use in the treatment of diseases and disorders associated with fatty acid metabolism, including obesity and other metabolic disorders.

Disorders that may be treated or prevented by an active compound or its salt or composition as described herein also include, but are not limited to: hereditary angioedema, capillary leak syndrome, hemolytic uremic syndrome (HUS), neurological disorders, Guillain Barre Syndrome, diseases of the central nervous system and other neurodegenerative conditions, glomerulonephritis (including membrane proliferative glomerulonephritis), SLE nephritis, proliferative nephritis, liver fibrosis, tissue regeneration and neural regeneration, or Barraquer-Simons Syndrome; inflammatory effects of sepsis, systemic inflammatory response syndrome (SIRS), disorders of inappropriate or undesirable complement activation, interleukin-2 induced toxicity during IL-2 therapy, inflammatory disorders, inflammation of autoimmune diseases, systemic lupus erythematosus (SLE), lupus nephritides, arthritis, immune complex disorders and autoimmune diseases, systemic lupus, or lupus erythematosus; ischemia/reperfusion injury (I/R injury), myocardial infarction, myocarditis, post-ischemic reperfusion conditions, balloon angioplasty, atherosclerosis, post-pump syndrome in cardiopulmonary bypass or renal bypass, renal ischemia, mesenteric artery reperfusion after aortic reconstruction, antiphospholipid syndrome, autoimmune heart disease, ischemia-reperfusion injuries, obesity, or diabetes; Alzheimer's dementia, stroke, schizophrenia, traumatic brain injury, trauma, Parkinson's disease, epilepsy, transplant rejection, prevention of fetal loss, biomaterial reactions (e.g. in hemodialysis, inplants), hyperacute allograft rejection, xenograft rejection, transplantation, psoriasis, burn injury, thermal injury including burns or frostbite, or crush injury; asthma, allergy, acute respiratory distress syndrome (ARDS), cystic fibrosis, adult respiratory distress syndrome, dyspnea, hemoptysis, chronic obstructive pulmonary disease (COPD), emphysema, pulmonary embolisms and infarcts, pneumonia, fibrogenic dust diseases, inert dusts and minerals (e.g., silicon, coal dust, beryllium, and asbestos), pulmonary fibrosis, organic dust diseases, chemical injury (due to irritant gases and chemicals, e.g., chlorine, phosgene, sulfur dioxide, hydrogen sulfide, nitrogen dioxide, ammonia, and hydrochloric acid), smoke injury, thermal injury (e.g., burn, freeze), bronchoconstriction, hypersensitivity pneumonitis, parasitic diseases, Goodpasture's Syndrome (anti-glomerular basement membrane nephritis), pulmonary vasculitis, Pauci-immune vasculitis, and immune complex-associated inflammation.

In some embodiments, a method for the treatment of sickle cell in a host is provided that includes the administration of an effective amount of an active compound or its salt or composition as described herein.

In some embodiments, a method for the treatment of immune thrombocytopenic purpura (ITP), thrombotic thrombocytopenic purpura (TTP), or idiopathic thrombocytopenic purpura (ITP) in a host is provided that includes the administration of an effective amount of an active compound or its salt or composition as described herein.

In some embodiments, a method for the treatment of ANCA-vasculitis in a host is provided that includes the administration of an effective amount of an active compound or its salt or composition as described herein.

In some embodiments, a method for the treatment of IgA nephropathy in a host is provided that includes the administration of an effective amount of an active compound or its salt or composition as described herein.

In some embodiments, a method for the treatment of rapidly progressing glomerulonephritis (RPGN), in a host is provided that includes the administration of an effective amount of an active compound or its salt or composition as described herein.

In some embodiments, a method for the treatment of lupus nephritis, in a host is provided that includes the administration of an effective amount of an active compound or its salt or composition as described herein.

In some embodiments, a method for the treatment of hemorraghic dengue fever, in a host is provided that includes the administration of an effective amount of an active compound or its salt or composition as described herein.

In an additional alternative embodiment, an active compound or its salt or composition as described herein is used in the treatment of an autoimmune disorder. The complement pathway enhances the ability of antibodies and phagocytic cells to clear microbes and damaged cells from the body. It is part of the innate immune system and in healthy individuals is an essential process. Inhibiting the complement pathway will decrease the body's immune system response. Therefore, it is an object of the present disclosure to treat autoimmune disorders by administering an effective does of an active compound or its salt or composition as described herein to a subject in need thereof.

In some embodiments, the autoimmune disorder is caused by activity of the complement system. In some embodiments the autoimmune disorder is caused by activity of the alternative complement pathway. In some embodiments the autoimmune disorder is caused by activity of the classical complement pathway. In another embodiment the autoimmune disorder is caused by a mechanism of action that is not directly related to the complement system, such as the over-proliferation of T-lymphocytes or the over-production of cytokines.

Non-limiting examples of autoimmune disorders include: lupus, allograft rejection, autoimmune thyroid diseases (such as Graves' disease and Hashimoto's thyroiditis), autoimmune uveoretinitis, giant cell arteritis, inflammatory bowel diseases (including Crohn's disease, ulcerative colitis, regional enteritis, granulomatous enteritis, distal ileitis, regional ileitis, and terminal ileitis), diabetes, multiple sclerosis, pernicious anemia, psoriasis, rheumatoid arthritis, sarcoidosis, and scleroderma.

In some embodiments, an active compound or its salt or composition as described herein is used in the treatment of lupus. Non-limiting examples of lupus include lupus erythematosus, cutaneous lupus, discoid lupus erythematosus, chilblain lupus erythematosus, and lupus erythematosus-lichen planus overlap syndrome.

Lupus erythematosus is a general category of disease that includes both systemic and cutaneous disorders. The systemic form of the disease can have cutaneous as well as systemic manifestations. However, there are also forms of the disease that are only cutaneous without systemic involvement. For example, SLE is an inflammatory disorder of unknown etiology that occurs predominantly in women, and is characterized by articular symptoms, butterfly erythema, recurrent pleurisy, pericarditis, generalized adenopathy, splenomegaly, as well as CNS involvement and progressive renal failure. The sera of most patients (over 98%) contain antinuclear antibodies, including anti-DNA antibodies. High titers of anti-DNA antibodies are essentially specific for SLE. Conventional treatment for this disease has been the administration of corticosteroids or immunosuppressants.

There are three forms of cutaneous lupus: chronic cutaneous lupus (also known as discoid lupus erythematosus or DLE), subacute cutaneous lupus, and acute cutaneous lupus. DLE is a disfiguring chronic disorder primarily affecting the skin with sharply circumscribed macules and plaques that display erythema, follicular plugging, scales, telangiectasia and atrophy. The condition is often precipitated by sun exposure, and the early lesions are erythematous, round scaling papules that are 5 to 10 mm in diameter and display follicular plugging. DLE lesions appear most commonly on the cheeks, nose, scalp, and ears, but they may also be generalized over the upper portion of the trunk, extensor surfaces of the extremities, and on the mucous membranes of the mouth. If left untreated, the central lesion atrophies and leaves a scar. Unlike SLE, antibodies against double-stranded DNA (e.g., DNA-binding test) are almost invariably absent in DLE.

Diabetes can refer to either type 1 or type 2 diabetes. In some embodiments an active compound or its salt or composition as described herein is provided at an effective dose to treat a patient with type 1 diabetes. In some embodiments an active compound or its salt or composition as described herein is provided at an effective dose to treat a patient with type 2 diabetes. Type 1 diabetes is an autoimmune disease. An autoimmune disease results when the body's system for fighting infection (the immune system) attacks a part of the body. In the case of diabetes type 1, the pancreas then produces little or no insulin.

In some embodiments, the complement-mediated disease or disorder comprises transplant rejection. In some embodiments, the complement-mediated disease or disorder is antibody-mediated transplant rejection.

In certain aspects, an active compound or its salt or composition as described herein is used to treat a proliferative disorder, including, but not limited to, cancer. Targeted cancers suitable for administration of an active compound or its salt described herein include, but are not limited to, estrogen-receptor positive cancer, HER2-negative advanced breast cancer, late-line metastatic breast cancer, liposarcoma, non-small cell lung cancer, liver cancer, ovarian cancer, glioblastoma, refractory solid tumors, retinoblastoma positive breast cancer as well as retinoblastoma positive endometrial, vaginal and ovarian cancers and lung and bronchial cancers, adenocarcinoma of the colon, adenocarcinoma of the rectum, central nervous system germ cell tumors, teratomas, estrogen receptor-negative breast cancer, estrogen receptor-positive breast cancer, familial testicular germ cell tumors, HER2-negative breast cancer, HER2-positive breast cancer, male breast cancer, ovarian immature teratomas, ovarian mature teratoma, ovarian monodermal and highly specialized teratomas, progesterone receptor-negative breast cancer, progesterone receptor-positive breast cancer, recurrent breast cancer, recurrent colon cancer, recurrent extragonadal germ cell tumors, recurrent extragonadal non-seminomatous germ cell tumor, recurrent extragonadal seminomas, recurrent malignant testicular germ cell tumors, recurrent melanomas, recurrent ovarian germ cell tumors, recurrent rectal cancer, stage III extragonadal non-seminomatous germ cell tumors, stage III extragonadal seminomas, stage III malignant testicular germ cell tumors, stage III ovarian germ cell tumors, stage IV breast cancers, stage IV colon cancers, stage IV extragonadal non-seminomatous germ cell tumors, stage IV extragonadal seminoma, stage IV melanomas, stage IV ovarian germ cell tumors, stage IV rectal cancers, testicular immature teratomas, testicular mature teratomas. In particular embodiments, the targeted cancers included estrogen-receptor positive, HER2-negative advanced breast cancer, late-line metastatic breast cancer, liposarcoma, non-small cell lung cancer, liver cancer, ovarian cancer, glioblastoma, refractory solid tumors, retinoblastoma positive breast cancer as well as retinoblastoma positive endometrial, vaginal and ovarian cancers and lung and bronchial cancers, metastatic colorectal cancer, metastatic melanoma with CDK4 mutation or amplification, or cisplatin-refractory, unresectable germ cell tumors, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, fibrosarcoma, myxosarcoma, chondrosarcoma, osteosarcoma, chordoma, malignant fibrous histiocytoma, hemangiosarcoma, angiosarcoma, lymphangiosarcoma, Mesothelioma, leiomyosarcoma, rhabdomyosarcoma, squamous cell carcinoma; epidermoid carcinoma, malignant skin adnexal tumors, adenocarcinoma, hepatoma, hepatocellular carcinoma, renal cell carcinoma, hypernephroma, cholangiocarcinoma, transitional cell carcinoma, choriocarcinoma, seminoma, embryonal cell carcinoma, glioma anaplastic; glioblastoma multiforme, neuroblastoma, medulloblastoma, malignant meningioma, malignant schwannoma, neurofibrosarcoma, parathyroid carcinoma, medullary carcinoma of thyroid, bronchial carcinoid, pheochromocytoma, Islet cell carcinoma, malignant carcinoid, malignant paraganglioma, melanoma, Merkel cell neoplasm, cystosarcoma phylloide, salivary cancers, thymic carcinomas, bladder cancer, and Wilms tumor, a blood disorder or a hematologic malignancy, including, but not limited to, myeloid disorder, lymphoid disorder, leukemia, lymphoma, myelodysplastic syndrome (MDS), myeloproliferative disease (MPD), mast cell disorder, and myeloma (e.g., multiple myeloma), among others, T-cell or NK-cell lymphoma, for example, but not limited to: peripheral T-cell lymphoma; anaplastic large cell lymphoma, for example anaplastic lymphoma kinase (ALK) positive, ALK negative anaplastic large cell lymphoma, or primary cutaneous anaplastic large cell lymphoma; angioimmunoblastic lymphoma; cutaneous T-cell lymphoma, for example mycosis fungoides, Sezary syndrome, primary cutaneous anaplastic large cell lymphoma, primary cutaneous CD30+ T-cell lymphoproliferative disorder; primary cutaneous aggressive epidermotropic CD8+ cytotoxic T-cell lymphoma; primary cutaneous gamma-delta T-cell lymphoma; primary cutaneous small/medium CD4+ T-cell lymphoma, and lymphomatoid papulosis; Adult T-cell Leukemia/Lymphoma (ATLL); Blastic NK-cell Lymphoma; Enteropathy-type T-cell lymphoma; Hematosplenic gamma-delta T-cell Lymphoma; Lymphoblastic Lymphoma; Nasal NK/T-cell Lymphomas; Treatment-related T-cell lymphomas; for example lymphomas that appear after solid organ or bone marrow transplantation; T-cell prolymphocytic leukemia; T-cell large granular lymphocytic leukemia; Chronic lymphoproliferative disorder of NK-cells; Aggressive NK cell leukemia; Systemic EBV+ T-cell lymphoproliferative disease of childhood (associated with chronic active EBV infection); Hydroa vacciniforme-like lymphoma; Adult T-cell leukemia/lymphoma; Enteropathy-associated T-cell lymphoma; Hepatosplenic T-cell lymphoma; or Subcutaneous panniculitis-like T-cell lymphoma.

In some embodiments, the methods described herein can be used to treat a host, for example a human, with a lymphoma or lymphocytic or myelocytic proliferation disorder or abnormality. For example, the methods as described herein can be administered to a host with a Hodgkin Lymphoma or a Non-Hodgkin Lymphoma. For example, the host can have a Non-Hodgkin Lymphoma such as, but not limited to: an AIDS-Related Lymphoma; Anaplastic Large-Cell Lymphoma; Angioimmunoblastic Lymphoma; Blastic NK-Cell Lymphoma; Burkitt's Lymphoma; Burkitt-like Lymphoma (Small Non-Cleaved Cell Lymphoma); Chronic Lymphocytic Leukemia/Small Lymphocytic Lymphoma; Cutaneous T-Cell Lymphoma; Diffuse Large B-Cell Lymphoma; Enteropathy-Type T-Cell Lymphoma; Follicular Lymphoma; Hepatosplenic Gamma-Delta T-Cell Lymphoma; Lymphoblastic Lymphoma; Mantle Cell Lymphoma; Marginal Zone Lymphoma; Nasal T-Cell Lymphoma; Pediatric Lymphoma; Peripheral T-Cell Lymphomas; Primary Central Nervous System Lymphoma; T-Cell Leukemias; Transformed Lymphomas; Treatment-Related T-Cell Lymphomas; or Waldenstrom's Macroglobulinemia, a Hodgkin Lymphoma, such as, but not limited to: Nodular Sclerosis Classical Hodgkin's Lymphoma (CHL); Mixed Cellularity CHL; Lymphocyte-depletion CHL; Lymphocyte-rich CHL; Lymphocyte Predominant Hodgkin Lymphoma; or Nodular Lymphocyte Predominant HL, a specific B-cell lymphoma or proliferative disorder such as, but not limited to: multiple myeloma; Diffuse large B cell lymphoma; Follicular lymphoma; Mucosa-Associated Lymphatic Tissue lymphoma (MALT); Small cell lymphocytic lymphoma; Mediastinal large B cell lymphoma; Nodal marginal zone B cell lymphoma (NMZL); Splenic marginal zone lymphoma (SMZL); Intravascular large B-cell lymphoma; Primary effusion lymphoma; or Lymphomatoid granulomatosis; B-cell prolymphocytic leukemia; Hairy cell leukemia; Splenic lymphoma/leukemia, unclassifiable; Splenic diffuse red pulp small B-cell lymphoma; Hairy cell leukemia-variant; Lymphoplasmacytic lymphoma; Heavy chain diseases, for example, Alpha heavy chain disease, Gamma heavy chain disease, Mu heavy chain disease; Plasma cell myeloma; Solitary plasmacytoma of bone; Extraosseous plasmacytoma; Primary cutaneous follicle center lymphoma; T cell/histiocyte rich large B-cell lymphoma; DLBCL associated with chronic inflammation; Epstein-Barr virus (EBV)+ DLBCL of the elderly; Primary mediastinal (thymic) large B-cell lymphoma; Primary cutaneous DLBCL, leg type; ALK+ large B-cell lymphoma; Plasmablastic lymphoma; Large B-cell lymphoma arising in HHV8-associated multicentric; Castleman disease; B-cell lymphoma, unclassifiable, with features intermediate between diffuse large B-cell lymphoma; or B-cell lymphoma, unclassifiable, with features intermediate between diffuse large B-cell lymphoma and classical Hodgkin lymphoma, a leukemia, for example, an acute or chronic leukemia of a lymphocytic or myelogenous origin, such as, but not limited to: Acute lymphoblastic leukemia (ALL); Acute myelogenous leukemia (AML); Chronic lymphocytic leukemia (CLL); Chronic myelogenous leukemia (CML); juvenile myelomonocytic leukemia (JMML); hairy cell leukemia (HCL); acute promyelocytic leukemia (a subtype of AML); large granular lymphocytic leukemia; or Adult T-cell chronic leukemia. In some embodiments, the patient has an acute myelogenous leukemia, for example an undifferentiated AML (M0); myeloblastic leukemia (M1; with/without minimal cell maturation); myeloblastic leukemia (M2; with cell maturation); promyelocytic leukemia (M3 or M3 variant [M3V]); myelomonocytic leukemia (M4 or M4 variant with eosinophilia [M4E]); monocytic leukemia (M5); erythroleukemia (M6); or megakaryoblastic leukemia (M7), small cell lung cancer, retinoblastoma, HPV positive malignancies like cervical cancer and certain head and neck cancers, MYC amplified tumors such as Burkitts' Lymphoma, and triple negative breast cancer; certain classes of sarcoma, certain classes of non-small cell lung carcinoma, certain classes of melanoma, certain classes of pancreatic cancer, certain classes of leukemia, certain classes of lymphoma, certain classes of brain cancer, certain classes of colon cancer, certain classes of prostate cancer, certain classes of ovarian cancer, certain classes of uterine cancer, certain classes of thyroid and other endocrine tissue cancers, certain classes of salivary cancers, certain classes of thymic carcinomas, certain classes of kidney cancers, certain classes of bladder cancers, and certain classes of testicular cancers.

In certain aspects, an active compound or its salt as described herein can be used to preserve or prevent damage to an organ or blood product. For example, an active compound or its salt described herein can be used to prevent damage to an organ, tissue, cell product, or blood product, that has been harvested for transplantation. In some embodiments, the organ is the heart, kidney, pancreas, lung, liver, or intestine. In some embodiments, the tissue is derived from the cornea, bone, tendon, muscle, heart valve, nerve, artery or vein, or the skin. In some embodiments, the blood product is whole blood, plasma, red blood cells or reticulocytes.

In some embodiments, an active compound or its salt or composition as described herein prevents or delays the onset of at least one symptom of a complement-mediated disease or disorder in an individual. In some embodiments, an active compound or its salt or composition as described herein reduces or eliminates at least one symptom of a complement-mediated disease or disorder in an individual. Examples of symptoms include, but are not limited to, symptoms associated with autoimmune disease, cancer, hematological disease, infectious disease, inflammatory disease, ischemia-reperfusion injury, neurodegenerative disease, neurodegenerative disorder, renal disease, transplant rejection, ocular disease, vascular disease, or a vasculitis disorder. The symptom can be a neurological symptom, for example, impaired cognitive function, memory impairment, loss of motor function, etc. The symptom can also be the activity of C1s protein in a cell, tissue, or fluid of an individual. The symptom can also be the extent of complement activation in a cell, tissue, or fluid of an individual.

In some embodiments, administering an active compound or its salt or composition as described herein to an individual modulates complement activation in a cell, tissue, or fluid of an individual. In some embodiments, administration of an active compound or its salt or composition as described herein to an individual inhibits complement activation in a cell, tissue, or fluid of an individual. For example, in some embodiments, an active compound or its salt or composition as described herein, when administered in one or more doses as monotherapy or in combination therapy to an individual having a complement-mediated disease or disorder, inhibits complement activation in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to complement activation in the individual before treatment with the compounds described herein.

In some embodiments, an active compound or its salt or composition as described herein reduces C3 deposition onto red blood cells; for example, in some embodiments, an an active compound or its salt or composition as described herein reduces deposition of C3b, iC3b, etc., onto RBCs. In some embodiments, an active compound or its salt or composition as described herein inhibits complement-mediated red blood cell lysis.

In some embodiments, an active compound or its salt or composition as described herein reduces C3 deposition onto platelets; for example, in some embodiments, an active compound or its salt or composition as described herein reduces deposition of C3b, iC3b, etc., onto platelets.

In some embodiments, administering an active compound or its salt or composition as described herein results in an outcome selected from the group consisting of: (a) a reduction in complement activation; (b) an improvement in cognitive function; (c) a reduction in neuron loss; (d) a reduction in phospho-Tau levels in neurons; (e) a reduction in glial cell activation; (f) a reduction in lymphocyte infiltration; (g) a reduction in macrophage infiltration; (h) a reduction in antibody deposition, (i) a reduction in glial cell loss; (j) a reduction in oligodendrocyte loss; (k) a reduction in dendritic cell infiltration; (I) a reduction in neutrophil infiltration; (m) a reduction in red blood cell lysis; (n) a reduction in red blood cell phagocytosis; (o) a reduction in platelet phagocytosis; (p) a reduction in platelet lysis; (q) an improvement in transplant graft survival; (r) a reduction in macrophage mediated phagocytosis; (s) an improvement in vision; (t) an improvement in motor control; (u) an improvement in thrombus formation; (v) an improvement in clotting; (w) an improvement in kidney function; (x) a reduction in antibody mediated complement activation; (y) a reduction in autoantibody mediated complement activation; (z) an improvement in anemia; (aa) reduction of demyelination; (ab) reduction of eosinophilia; (ac) a reduction of C3 deposition on red blood cells (e.g., a reduction of deposition of C3b, iC3b, etc., onto RBCs); and (ad) a reduction in C3 deposition on platelets (e.g., a reduction of deposition of C3b, iC3b, etc., onto platelets); and (ae) a reduction of anaphylatoxin toxin production; (af) a reduction in autoantibody mediated blister formation; (ag) a reduction in autoantibody induced pruritis; (ah) a reduction in autoantibody induced erythematosus; (ai) a reduction in autoantibody mediated skin erosion; (aj) a reduction in red blood cell destruction due to transfusion reactions; (ak) a reduction in red blood cell lysis due to alloantibodies; (al) a reduction in hemolysis due to transfusion reactions; (am) a reduction in allo-antibody mediated platelet lysis; (an) a reduction in platelet lysis due to transfusion reactions; (ao) a reduction in mast cell activation; (ap) a reduction in mast cell histamine release; (aq) a reduction in vascular permeability; (ar) a reduction in edema; (as) a reduction in complement deposition on transplant graft endothelium; (at) a reduction of anaphylatoxin generation in transplant graft endothelium; (au) a reduction in the separation of the dermal-epidermal junction; (av) a reduction in the generation of anaphylatoxins in the dermal-epidermal junction; (aw) a reduction in alloantibody mediated complement activation in transplant graft endothelium; (ax) a reduction in antibody mediated loss of the neuromuscular junction; (ay) a reduction in complement activation at the neuromuscular junction; (az) a reduction in anaphylatoxin generation at the neuromuscular junction; (ba) a reduction in complement deposition at the neuromuscular junction; (bb) a reduction in paralysis; (bc) a reduction in numbness; (bd) increased bladder control; (be) increased bowel control; (bf) a reduction in mortality associated with autoantibodies; and (bg) a reduction in morbidity associated with autoantibodies.

In some embodiments, an active compound or its salt or composition as described herein, when administered in one or more doses as monotherapy or in combination therapy to an individual having a complement-mediated disease or disorder, is effect to achieve a reduction of at least about 10%, at least about 15%, at least about 20%, at least about 25%, at least about 30%, at least about 40%, at least about 50%, at least about 60%, at least about 70%, at least about 80%, at least about 90%, or more than 90%, of one or more of the following outcomes: (a) complement activation; (b) decline in cognitive function; (c) neuron loss; (d) phospho-Tau levels in neurons; (e) glial cell activation; (f) lymphocyte infiltration; (g) macrophage infiltration; (h) antibody deposition, (i) glial cell loss; (j) oligodendrocyte loss; (k) dendritic cell infiltration; (I) neutrophil infiltration; (m) red blood cell lysis; (n) red blood cell phagocytosis; (o) platelet phagocytosis; (p) platelet lysis; (q) transplant graft rejection; (r) macrophage mediated phagocytosis; (s) vision loss; (t) antibody mediated complement activation; (u) autoantibody mediated complement activation; (v) demyelination; (w) eosinophilia; compared to the level or degree of the outcome in the individual before treatment with the active compound.

In some embodiments, an active compound or its salt or composition as described herein, when administered in one or more doses as monotherapy or in combination therapy to an individual having a complement-mediated disease or disorder, is effect to achieve an improvement of at least about 10%, at least about 15%, at least about 20%, at least about 25%, at least about 30%, at least about 40%, at least about 50%, at least about 60%, at least about 70%, at least about 80%, at least about 90%, or more than 90%, of one or more of the following outcomes: a) cognitive function; b) transplant graft survival; c) vision; d) motor control; e) thrombus formation; f) clotting; g) kidney function; and h) hematocrit (red blood cell count), compared to the level or degree of the outcome in the individual before treatment with the active compound.

In some embodiments, administering an active compound or its salt or composition as described herein to an individual reduces complement activation in the individual. For example, in some embodiments, an active compound or its salt or composition as described herein, when administered in one or more doses as monotherapy or in combination therapy to an individual having a complement-mediated disease or disorder, reduces complement activation in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to complement activation in the individual before treatment with the active compound or its salt.

In some embodiments, administering an active compound or its salt or composition as described herein improves cognitive function in the individual. For example, in some embodiments, an active compound described herein, when administered in one or more doses as monotherapy or in combination therapy to an individual having a complement-mediated disease or disorder, improves cognitive function in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to the cognitive function in the individual before treatment with the active compound.

In some embodiments, administering an active compound or its salt or composition as described herein reduces the rate of decline in cognitive function in the individual. For example, in some embodiments, an active compound or its salt, when administered in one or more doses as monotherapy or in combination therapy to an individual having a complement-mediated disease or disorder, reduces the rate of decline of cognitive function in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to the rate of decline in cognitive function in the individual before treatment with the active compound or its salt.

In some embodiments, administering an active compound or its salt or composition as described herein to an individual reduces neuron loss in the individual. For example, in some embodiments, an active compound or its salt, when administered in one or more doses as monotherapy or in combination therapy to an individual having a complement-mediated disease or disorder, reduces neuron loss in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to neuron loss in the individual before treatment with the active compound.

In some embodiments, administering an active compound or its salt or composition as described herein to an individual reduces phospho-Tau levels in the individual. For example, in some embodiments, an active compound or its salt, when administered in one or more doses as monotherapy or in combination therapy to an individual having a complement-mediated disease or disorder, reduces phospho-Tau in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to the phospho-Tau level in the individual before treatment with the active compound or its salt.

In some embodiments, administering an active compound or its salt or composition as described herein to an individual reduces glial cell activation in the individual. For example, in some embodiments, an active compound or its salt, when administered in one or more doses as monotherapy or in combination therapy to an individual having a complement-mediated disease or disorder, reduces glial activation in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to glial cell activation in the individual before treatment with the active compound or its salt. In some embodiments, the glial cells are astrocytes or microglia.

In some embodiments, administering an active compound or its salt or composition as described herein to an individual reduces lymphocyte infiltration in the individual. For example, in some embodiments, an active compound or its salt, when administered in one or more doses as monotherapy or in combination therapy to an individual having a complement-mediated disease or disorder, reduces lymphocyte infiltration in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to lymphocyte infiltration in the individual before treatment with the active compound or its salt.

In some embodiments, administering an active compound or its salt or composition as described herein to an individual reduces macrophage infiltration in the individual. For example, in some embodiments, an active compound or its salt, when administered in one or more doses as monotherapy or in combination therapy to an individual having a complement-mediated disease or disorder, reduces macrophage infiltration in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to macrophage infiltration in the individual before treatment with the active compound or its salt.

In some embodiments, administering an active compound or its salt or composition as described herein to an individual reduces antibody deposition in the individual. For example, in some embodiments, an active compound or its salt, when administered in one or more doses as monotherapy or in combination therapy to an individual having a complement-mediated disease or disorder, reduces antibody deposition in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to antibody deposition in the individual before treatment with the active compound or its salt.

In some embodiments, administering an active compound or its salt or composition as described herein to an individual reduces anaphylatoxin (e.g., C3a, C4a, C5a) production in an individual. For example, in some embodiments, an active compound or its salt, when administered in one or more doses as monotherapy or in combination therapy to an individual having a complement-mediated disease or disorder, reduces anaphylatoxin production in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to the level of anaphylatoxin production in the individual before treatment with the active compound or its salt.

The present disclosure provides for use of an active compound or its salt of the present disclosure or a pharmaceutical composition comprising an active compound or its salt of the present disclosure and a pharmaceutically acceptable excipient to treat an individual having a complement-mediated disease or disorder. In some embodiments, the present disclosure provides for use of an active compound or its salt of the present disclosure to treat an individual having a complement-mediated disease or disorder. In some embodiments, the present disclosure provides for use of a pharmaceutical composition comprising an active compound or its salt of the present disclosure and a pharmaceutically acceptable excipient to treat an individual having a complement-mediated disease or disorder.

### Combination Therapy

In one aspect of the present disclosure, an active compound or its salt or composition as described herein may be provided in combination or alternation with or preceded by, concomitant with or followed by, an effective amount of at least one additional therapeutic agent, for example, for treatment of a disorder listed herein. Non-limiting examples of second active agents for such combination therapy are provided as follows.

In some embodiments, an active compound or its salt or composition as described herein may be provided in combination or alternation with at least one additional inhibitor of the complement system or a second active compound with a different biological mechanism of action. In the description below and herein generally, whenever any of the terms referring to an active compound or its salt or composition as described herein are used, it should be understood that pharmaceutically acceptable salts, prodrugs or compositions are considered included, unless otherwise stated or inconsistent with the text.

In non-limiting embodiments, an active compound or its salt or composition as described herein may be provided together with a protease inhibitor, a soluble complement regulator, a therapeutic antibody (monoclonal or polyclonal), complement component inhibitor, receptor agonist, chemotherapeutic agent, or siRNA.

In other embodiments, an active compound described herein is administered in combination or alternation with an antibody against tumor necrosis factor (TNF), including but not limited to infliximab (REMICADE®), adalimumab (HUMIRA®), certolizumab (CIMZIA®), golimumab (SIMPONI®), or a receptor fusion protein such as etanercept (ENBREL®). In some embodiments, the agent for combination therapy is a biosimilar of any agent named above, including, but not limited to, REMISMA® (infliximab biosimilar), FLIXABI® (infliximab biosimilar), AMGEVITA® (adalimumab biosimilar), IMRALDI® (adalimumab biosimilar), CYTELZO® (adalimumab biosimilar), BENEPALI® (etanercept biosimilar), and ERELZI® (etanercept biosimilar).

In another embodiment, an active compound as described herein can be administered in combination or alternation with an anti-CD20 antibody, including but not limited to rituximab (RITUXAN®), ofatumumab (ARZERRA®), tositumomab (BEXXAR®), obinutuzumab (GAZYVA®), ibritumomab (ZEVALIN®), ocrelizumab (OCREVUS®), or veltuzumab. In some embodiments, the agent for combination therapy is a biosimilar of any agent named above, including, but not limited to, TRUXIMA® (rituximab biosimilar).

In an alternative embodiment, an active compound as described herein can be administered in combination or alternation with an anti-IL6 antibody, including but not limited to tocilizumab (ACTEMRA®), siltuximab (SYLVANT®), sarilumab (KEVZARA®), sirukumab, clazakizumab, vobarilizumab, olokizumab, and WBP216 (MEDI5117). In some embodiments, the agent for combination therapy is a biosimilar of any agent named above, including, but not limited to, BAT1806 (tocilizumab biosimilar).

In an alternative embodiment, an active compound as described herein can be administered in combination or alternation with an IL17 inhibitor, including but not limited to secukinumab (Cosentyx), ixekizumab (TALTZ®), brodalumab (SILIQ®), bimekizumab, ALX-0761, CJM112, CNT06785, LY3074828, SCH-900117, and MSB0010841. In some embodiments, the agent for combination therapy is a biosimilar of any agent named above.

In an alternative embodiment, an active compound as described herein can be administered in combination or alternation with a p40 (IL12/IL23) inhibitor, including but not limited to ustekinumab (STELARA®) and briakinumab (ABT874). In some embodiments, the agent for combination therapy is a biosimilar of any agent named above, including, but not limited to, FYB202 (ustekinumab biosimilar) and Neulara® (ustekinumab biosimilar).

In an alternative embodiment, an active compound as described herein can be administered in combination or alteration with an IL23 inhibitor, including but not limited to risankizumab (SKYRIZI®), tildrakizumab (ILUMYA®), guselkumab (TREMFYA®), mirakizumab and brazikumab. In some embodiments, the agent for combination therapy is a biosimilar of any agent named above.

In an alternative embodiment, an active compound as described herein can be administered in combination or alteration with an anti-interferon α antibody, for example but not limited to sifalimumab, anifrolumab, and rontalizumab. In some embodiments, the agent for combination therapy is a biosimilar of any agent named above.

In an alternative embodiment, an active compound as described herein can be administered in combination or alteration with a kinase inhibitor, for example but not limited to a JAK1/JAK3 inhibitor, for example but not limited to tofacitinib (XELJANZ®). In an alternative embodiment, an active compound as described herein can be administered in combination or alteration with a JAK1/JAK2 inhibitor, for example but not limited to baracitinib (OLUMIANT®) and ruxolitinib (JAKAFI®).

In an alternative embodiment, an active compound as described herein can be administered in combination or alteration with an anti-VEGF agent, for example but not limited to: aflibercept (EYLEA®; Regeneron Pharmaceuticals); ranibizumab (LUCENTIS®: Genentech and Novartis); pegaptanib (MACUGEN®; OSI Pharmaceuticals and Pfizer); bevacizumab (AVASTIN®; Genentech/Roche) and ziv-aflibercept (ZALTRAP®).

In an alternative embodiment, an active compound as described herein can be administered in combination or alternation with a tyrosine kinase inhibitor, for example but not limited to: lapatinib (TYKERB®); sunitinib (SUTENT®); axitinib (INLYTA®); pazopanib; sorafenib (NEXAVAR®); ponatinib (INCLUSIG®); regorafenib (STIVARGA®); cabozantinib (ABOMETYX®; COMETRIQ®); vendetanib (CAPRELSA®); ramucirumab (CYRAMZA®); lenvatinib (LENVIMA®); cediranib (RECENTIN®); anecortane acetate, squalamine lactate, and corticosteroids.

In another embodiment, an active compound as described herein can be administered in combination or alternation with an immune checkpoint inhibitor. Non-limiting examples of checkpoint inhibitors include anti-PD-1 or anti-PDL1 antibodies, for example, nivolumab (OPDIVO®), pembrolizumab (KEYTRUDA®), pidilizumab, AMP-224 (AstraZeneca and Medlmmune), PF-06801591 (Pfizer), MED10680 (AstraZeneca), PDR001 (Novartis), REGN2810 (Regeneron), SHR-12-1 (Jiangsu Hengrui Medicine Company and Incyte Corporation), TSR-042 (Tesaro), and the PD-L1/VISTA inhibitor CA-170 (Curis Inc.), atezolizumab (TECENTRIQ®), durvalumab (IMFINZI®), and KN035, or anti-CTLA4 antibodies, for example Ipilimumab (YERVOY®), Tremelimumab, AGEN1884 and AGEN2041 (Agenus).

Non-limiting examples of active agents that can be used in combination with active compounds described herein include, but are not limited to:
Protease inhibitors: plasma-derived C1-INH concentrates, for example CETOR® (Sanquin), BERINERT-P® (CSL Behring, Lev Pharma), HAEGARDA® (CSL Bering), CINRYZE®; recombinant human C1-inhibitors, for example RHUCIN®; ritonavir (NORVIR®, Abbvie, Inc.);
Soluble complement regulators: Soluble complement receptor 1 (TP10) (Avant Immunotherapeutics); sCR1-sLex/TP-20 (Avant Immunotherapeutics); MLN-2222/CAB-2 (Millenium Pharmaceuticals); Mirococept (Inflazyme Pharmaceuticals);
Therapeutic antibodies: Eculizumab/SOLIRIS® (Alexion Pharmaceuticals); Pexelizumab (Alexion Pharmaceuticals); Ravulizumab/ULTOMIRIS® (Alexion Pharmaceuticals); BCD-148 (Biocad); ABP-959 (Amgen); SB-12 (Samsung Bioepsis); Ofatumumab (Genmab A/S); TNX-234 (Tanox); TNX-558 (Tanox); TA106 (Taligen Therapeutics); Neutrazumab (G2 Therapies); Anti-properdin (Novelmed Therapeutics); HuMax-CD38 (Genmab A/S); Anti-properdin compounds from WO 2018/140956 (Alexion Pharmaceuticals);
Complement component inhibitors: Compstatin/POT-4 (Potentia Pharmaceuticals); ARC1905 (Archemix); 4(1 MEW)APL-1, APL-2 (Apellis); CP40/AMY-101, PEG-Cp40 (Amyndas); eculizumab/SOLIRIS® (Alexion Pharmaceuticals); Pexelizumab (Alexion Pharmaceuticals); ravulizumab/ULTOMIRIS® (Alexion Pharmaceuticals);
Multiple kinase inhibitors: Sorafenib Tosylate (NEXAVAR®); Imatinib Mesylate (GLEEVEC®); Sunitinib Malate (SUTENT®); Ponatinib (ICLUSIG®); Axitinib (INLYTA®); Nintedanib (OFEV®); Pazopanib HCl (VOTRIENT®); Dovitinib (TKI-258, Oncology Ventures); gilteritnib (XOSPATA®); Linifanib (ABT-869); Crenolanib (CP-868596); Masitinib (AB1010); Tivozanib (FOTIVDA®); Motesanib Diphosphate (AMG-706); Amuvatinib (MP-470); TSU-68 (SU6668, Orantinib); CP-673451; Ki8751; Telatinib (BAY 57-9352); PP121; KRN 633; MK-2461; Tyrphostin (AG 1296); Sennoside B; AZD2932; and Trapidil;
Anti-factor H or anti-factor B agents: Anti-FB siRNA (Alnylam); FCFD4514S (Genentech/Roche) SOMAmers for CFB and CFD (SomaLogic); TA106 (Alexion Pharmaceuticals); 5C6, NM8074 (Novelmed) and AMY-301 (Amyndas);
Complement C3 or CAP C3 Convertase targeting molecules: TT30 (CR2/CFH) (Alexion); TT32 (CR2/CR1) (Alexion Pharmaceuticals); Nafamostat (FUT-175, Futhan) (Torri Pharmaceuticals); Bikaciomab, NM9308 (Novelmed); CVF, HC-1496 (InCode) ALXN1102/ALXN1103 (TT30) (Alexion Pharmaceuticals); rFH (Optherion); H17 C3 (C3b/iC3b) (EluSys Therapeutics); Mini-CFH (Amyndas) Mirococept (APT070); sCR1 (CDX-1135) (Celldex); CRIg/CFH; Anti-CR3, anti-MASP2, anti-MASP3, anti C1s, and anti-C1n molecules: CINRYZE®(Takeda); TNT003 (Bioverativ/Sanofi); BIVV009 (fka TNT009; Bioverativ/Sanofi); BIVV020 (Bioverativ/Sanofi); OMS721 (Omeros); and OMS906 (Omeros);
Factor B and Factor Bb inhibitors: IONIS-FB-LRx (lonis Pharmaceuticals); for example, as described in US Patent Publication 20190071492 to Allergan, International Publication WO2017176651 to True North Therapeutics (now Sanofi), U.S. Pat. No. 9,243,070 (Novelmed); NM8074 (Novelmed); and as further described below;
Plasma kallikrein inhibitors: KALBITOR® and TAKHZYRO®;
Bradykinin receptor antagonists: FIRAZYR®;
Factor D inhibitors, as further described below.
Receptor agonists: PMX-53 (Peptech Ltd.); JPE-137 (Jerini); JSM-7717 (Jerini);
Others: Recombinant human MBL (rhMBL; Enzon Pharmaceuticals); Imides and glutarimide derivatives such as thalidomide, lenalidomide, pomalidomide; Additional non-limiting examples that can be used in combination or alternation with an active compound or its salt or composition as described herein include the following.

Non-limiting examples for combination therapy

| Name | Target | Company | Class of Molecule |
|---|---|---|---|
| LFG316 | C5 | Novartis/Morphosys | Monoclonal antibody |
| SB12 | C5 | Samsung Bioepsis | Monoclonal antibody |
| IONIS-FB-LRx | CFB | Ionis Pharmaceuticals | Antisense Inhibitor |
| 4(1MEW)APL-1,APL-2 | C3/C3b | Apellis | Compstatin Family |
| 4(1MeW)POT-4 | C3/C3b | Potentia | Compstatin Family |
| ALN-CC5/cemdisiran | C5 | Alnylam | SiRNA |
| Anti-FB siRNA | CFB | Alnylam | SiRNA |
| ARC1005 | C5 | Novo Nordisk | Aptamers |
| ATA | C5 | N.A. | Chemical |
| BIVV009 | C1s | Bioverativ/Sanofi | Monoclonal antibody |
| BERINERT ® | C1n/C1s | CSL Bering | Human purified protein |
| CCX168 (avocopan) | C5 | ChemoCentryx | Ligand |
| Coversin (nomacopan) | C5 | Akari Therapeutics | recombinant small protein |
| CP40/AMY-101, PEG- | C3/C3b | Amyndas | Compstatin Family |
| Cp40 | | | |
| CRIg/CFH | CAP C3 | NA | CFH-based protein |
| | convertase | | |
| CINRYZE ® | C1n/C1s | Takeda | Human purified protein |
| FCFD4514S | CFD | Genentech/Roche | Monoclonal antibody |
| FIRAZYR ® | Bradykinin | Takeda Pharmaceuticals | peptidomimetic |
| H17 | C3 (C3b/iC3b) | EluSys Therapeutics | Monoclonal antibody |
| IFX-1 (CaCP29) | C5a | InflaRx | Monoclonal antibody |
| KALBITOR ® | kallikrein | Shire Pharmaceuticals | polypeptide |
| Mini-CFH | CAP C3 | Amyndas | CFH-based protein |
| | convertase | | |
| Mirococept (APT070) | CAP and CCP | NA | CR1-based protein |
| | C3 | | |
| Mubodine | C5 | Adienne | Monoclonal antibody |
| RA101348 (zilucoplan) | C5 | Ra Pharma | Small molecule |
| RUCONEST ® | C1n/C1s | Pharming | Recombinant human |
| | | | protein |
| SCR1 (CDX-1135) | CAP and CP C3 | Celldex | CR1-based protein |
| SOBI002 | C5 | Swedish Orphan Biovitrum | Affibody |
| SOMAmers | C5 | SomaLogic | Aptamers |
| SOMAmers | CFB and CFD | SomaLogic | Aptamers |
| TAKHZYRO ® | kallakrein | Shire Pharmaceuticals | Monoclonal antibody |
| TA106 | CFB | Alexion Pharmaceuticals | Monoclonal antibody |
| TNT003 | C1s | Bioverativ/Sanofi | Monoclonal antibody |
| TT30 (CR2/CFH) | CAP C3 | Alexion Pharmaceuticals | CFH-based protein |
| | convertase | | |
| TT32 (CR2/CR1) | CAP and CCP | Alexion Pharmaceuticals | CR1-based protein |
| | C3 | | |
| Nafamostat (FUT-175, | C1s, CFD, other | Torri Pharmaceuticals | Small molecule |
| Futhan) | proteases | | |
| OMS721 | MASP-2 | Omeros | Monoclonal antibody |
| OMS906 | MASP-3 | Omeros | Monoclonal antibody |
| NM8074 | CFB | Novelmed | Monoclonal antibody |
| Bikaciomab, NM9308 | CFB | Novelmed | Monoclonal antibody |
| NM9401 | Properdin | Novelmed | Monoclonal antibody |
| CVF, HC-1496 | C3 | InCode | Recombinant peptide |
| ALXN1102/ALXN1103 | C3-conv, C3b | Alexion Pharmaceuticals | Regulator |
| (TT30) | | | |
| rFH | C3-conv, C3b | Optherion | Regulator |
| 5C6, AMY-301 | CFH | Amyndas | Regulator |
| Erdigna | C5 | Adienne Pharma | Antibody |
| ARC1905 | C5 | Ophthotech | Monoclonal Antibody |
| MEDI7814 | C5/C5a | MedImmune | Monoclonal Antibody |
| NOX-D19 | C5a | Noxxon | Aptamer (Spiegelmer) |
| PMX53, PMX205 | C5aR | Cephalon, Teva | Peptidomimetic |
| CCX168 | C5aR | ChemoCentryx | Small molecule |
| ADC-1004 | C5aR | Alligator Bioscience | Small molecule |
| Anti-C5aR-151, NN8209; | C5aR | Novo Nordisk | Monoclonal Antibody |
| Anti-C5aR-215, NN8210 | | | |
| Imprime PGG | CR3 | Biothera | Soluble beta-glucan |
| ANX005; ANX007 | C1q | Annexon | Monoclonal Antibody |
| LNP023 | fB | Novartis | Small molecule |
| Lampalizumab | fD | Roche | Monoclonal Antibody |
| avacincaptad pegol | C5 | Ophthotech | Aptamer |
| Regenemab | C6 | Regenesance | Monoclonal Antibody |
| BIVV020 | C1s | Bioverativ | Monoclonal Antibody |
| PRO-02 | C2 | Broteio/Argen-x | Monoclonal Antibody |
| 5C6, compsorbin | fH | Amyndas | Peptide |
| SOBI005 | C5 | Sobi | Protein |
| ISU305 | C5 | ISU ABXIS | Monoclonal Antibody |
| IFX-2, IFX-3 | C5a | InflaRx | Monoclonal Antibody |
| ALS-205 | C5aR1 | Alsonex | Peptide |
| DF2593A | C5aR1 | Dompé | Small Molecule |
| IPH5401 | C5aR1 | Innate Pharma | Monoclonal Antibody |
| C6-LNA | C6 | Regenesance | Oligonucleotide |
| Pozelimab (REGN3918) | C5 | Regeneron | Monoclonal Antibody |
| SKY59 (crovalimab) | C5 | Roche/Chugai | Monoclonal Antibody |
| Aptamers to Factor D | fD | Vitrisa Therapeutics | Aptamer |
| CLG561 | Properdin | Novartis | Monoclonal Antibody |
| Tesidolumab; LFG316 | C5 | Novartis and | Monoclonal Antibody |
| | | MorphoSys | |

In some embodiments, the agent for combination therapy is a biosimilar of any agent named above.

In some embodiments, an active compound or its salt or composition as described herein may be provided together with a compound that inhibits an enzyme that metabolizes an administered protease inhibitor. In some embodiments, a compound or salt may be provided together with ritonavir.

In some embodiments, an active compound or its salt or composition as described herein may be provided in combination with a terminal complement inhibitor, for example a complement C5 inhibitor or C5 convertase inhibitor. In another embodiment, an active compound or its salt or composition as described herein may be provided in combination with eculizumab, a monoclonal antibody directed to the complement factor C5 and manufactured and marketed by Alexion Pharmaceuticals under the tradename SOLIRIS®. Eculizumab has been approved by the U.S. FDA for the treatment of PNH and aHUS. In another embodiment, an active compound or its salt or composition as described herein may be provided in combination with revulizumab, a monoclonal antibody directed to the complement factor C5 and manufactured and marketed by Alexion Pharmaceuticals under the tradename ULTOMIRIS®. Revulizumab has been approved by the U.S. FDA for the treatment of PNH. Additional C5 and C5 convertase inhibitors include, but are not limited to, cemdisiran (Alnylam); prozelimab (Regeneron); BCD-148 (Biocad); ABP-959 (Amgen); SB-12 (Samsung Bioepis Co., Ltd.); LFG316 (Novartis); coversin (nomacopan; Akari)); zilucoplan (Ra Pharma); crovalimab (SKY59; Roche/Chugai); and mubodina (Adienne Pharma).

In some embodiments, an active compound or its salt or composition as described herein is administered in combination with an anti-inflammatory drug, antimicrobial agent, anti-angiogenesis agent, immunosuppressant, antibody, steroid, ocular antihypertensive drug or combinations thereof. Examples of such agents include amikacin, anecortane acetate, anthracenedione, anthracycline, an azole, amphotericin B, bevacizumab, camptothecin, cefuroxime, chloramphenicol, chlorhexidine, chlorhexidine digluconate, clortrimazole, a clotrimazole cephalosporin, corticosteroids, dexamethasone, desamethazone, econazole, eftazidime, epipodophyllotoxin, fluconazole, flucytosine, fluoropyrimidines, fluoroquinolines, gatifloxacin, glycopeptides, imidazoles, itraconazole, ivermectin, ketoconazole, levofloxacin, macrolides, miconazole, miconazole nitrate, moxifloxacin, natamycin, neomycin, nystatin, ofloxacin, polyhexamethylene biguanide, prednisolone, prednisolone acetate, pegaptanib, platinum analogs, polymicin B, propamidine isethionate, pyrimidine nucleoside, ranibizumab, squalamine lactate, sulfonamides, triamcinolone, triamcinolone acetonide, triazoles, vancomycin, anti-vascular endothelial growth factor (VEGF) agents, VEGF antibodies, VEGF antibody fragments, vinca alkaloid, timolol, betaxolol, travoprost, latanoprost, bimatoprost, brimonidine, dorzolamide, acetazolamide, pilocarpine, ciprofloxacin, azithromycin, gentamycin, tobramycin, cefazolin, voriconazole, gancyclovir, cidofovir, foscarnet, diclofenac, nepafenac, ketorolac, ibuprofen, indomethacin, fluoromethalone, rimexolone, anecortave, cyclosporine, methotrexate, tacrolimus, anti-PDGFR molecule, and combinations thereof.

In some embodiments of the present disclosure, an active compound or its salt or composition as described herein can be administered in combination or alternation with at least one immunosuppressive agent. The immunosuppressive agent as non-limiting examples, may be a calcineurin inhibitor, e.g. a cyclosporin or an ascomycin, e.g. Cyclosporin A (NEORAL®), FK506 (tacrolimus), pimecrolimus, a mTOR inhibitor, e.g. rapamycin or a derivative thereof, e.g. Sirolimus (RAPAMUNE®), Everolimus (Certican®), temsirolimus, zotarolimus, biolimus-7, biolimus-9, a rapalog, e.g. ridaforolimus, azathioprine, campath 1H, a S1P receptor modulator, e.g. fingolimod or an analog thereof, an anti IL-8 antibody, mycophenolic acid or a salt thereof, e.g. sodium salt, or a prodrug thereof, e.g. Mycophenolate Mofetil (CELLCEPT®), OKT3 (ORTHOCLONE OKT3®), Prednisone, ATGAM®, THYMOGLOBULIN®, Brequinar Sodium, OKT4, T10B9.A-3A, 33B3.1, 15-deoxyspergualin, tresperimus, Leflunomide ARAVA®, CTLAI-Ig, anti-CD25, anti-IL2R, Basiliximab (SIMULECT®), Daclizumab (ZENAPAX®), mizorbine, methotrexate, dexamethasone, ISAtx-247, SDZ ASM 981 (pimecrolimus, ELIDEI®), CTLA4Ig (Abatacept), belatacept, LFA3Ig, etanercept (sold as ENBREL® by Immunex), adalimumab (HUMIRA®), infliximab (REMICADE®), an anti-LFA-1 antibody, natalizumab (ANTEGREN®), Enlimomab, gavilimomab, antithymocyte immunoglobulin, siplizumab, Alefacept efalizumab, pentasa, mesalazine, asacol, codeine phosphate, benorylate, fenbufen, naprosyn, diclofenac, etodolac and indomethacin, tocilizumab (Actemra), siltuximab (Sylvant), secukibumab (Cosentyx), ustekinumab (Stelara), risankizumab, sifalimumab, aspirin and ibuprofen.

Examples of anti-inflammatory agents include methotrexate, dexamethasone, dexamethasone alcohol, dexamethasone sodium phosphate, fluromethalone acetate, fluromethalone alcohol, lotoprendol etabonate, medrysone, prednisolone acetate, prednisolone sodium phosphate, difluprednate, rimexolone, hydrocortisone, hydrocortisone acetate, lodoxamide tromethamine, aspirin, ibuprofen, suprofen, piroxicam, meloxicam, flubiprofen, naproxan, ketoprofen, tenoxicam, diclofenac sodium, ketotifen fumarate, diclofenac sodium, nepafenac, bromfenac, flurbiprofen sodium, suprofen, celecoxib, naproxen, rofecoxib, glucocorticoids, diclofenac, and any combination thereof. In some embodiments, an active compound or its salt or composition as described herein is combined with one or more non-steroidal anti-inflammatory drugs (NSAIDs) selected from naproxen sodium (Anaprox), celecoxib (Celebrex), sulindac (Clinoril), oxaprozin (Daypro), salsalate (Disalcid), diflunisal (Dolobid), piroxicam (Feldene), indomethacin (Indocin), etodolac (Lodine), meloxicam (Mobic), naproxen (Naprosyn), nabumetone (Relafen), ketorolac tromethamine (Toradol), naproxen/esomeprazole (Vimovo), and diclofenac (Voltaren), and combinations thereof.

In some embodiments, an active compound or its salt or composition as described herein is administered in combination or alteration with an omega-3 fatty acid or a peroxisome proliferator-activated receptor (PPARs) agonist. Omega-3 fatty acids are known to reduce serum triglycerides by inhibiting DGAT and by stimulating peroxisomal and mitochondrial beta oxidation. Two omega-3 fatty acids, eicosapentaenoic acid (EPA) and docosahexaenoic acid (DHA), have been found to have high affinity for both PPAR-alpha and PPAR-gamma. Marine oils, e.g., fish oils, are a good source of EPA and DHA, which have been found to regulate lipid metabolism. Omega-3 fatty acids have been found to have beneficial effects on the risk factors for cardiovascular diseases, especially mild hypertension, hypertriglyceridemia and on the coagulation factor VII phospholipid complex activity. Omega-3 fatty acids lower serum triglycerides, increase serum HDL-cholesterol, lower systolic and diastolic blood pressure and the pulse rate, and lower the activity of the blood coagulation factor VII-phospholipid complex. Further, omega-3 fatty acids seem to be well tolerated, without giving rise to any severe side effects. One such form of omega-3 fatty acid is a concentrate of omega-3, long chain, polyunsaturated fatty acids from fish oil containing DHA and EPA and is sold under the trademark OMACOR®. Such a form of omega-3 fatty acid is described, for example, in U.S. Pat. Nos. 5,502,077, 5,656,667 and 5,698,594, the disclosures of which are incorporated herein by reference.

Peroxisome proliferator-activated receptors (PPARs) are members of the nuclear hormone receptor superfamily ligand-activated transcription factors that are related to retinoid, steroid and thyroid hormone receptors. There are three distinct PPAR subtypes that are the products of different genes and are commonly designated PPAR-alpha, PPAR-beta/delta (or merely, delta) and PPAR-gamma. General classes of pharmacological agents that stimulate peroxisomal activity are known as PPAR agonists, e.g., PPAR-alpha agonists, PPAR-gamma agonists and PPAR-delta agonists. Some pharmacological agents are combinations of PPAR agonists, such as alpha/gamma agonists, etc., and some other pharmacological agents have dual agonist/antagonist activity. Fibrates such as fenofibrate, bezafibrate, clofibrate and gemfibrozil, are PPAR-alpha agonists and are used in patients to decrease lipoproteins rich in triglycerides, to increase HDL and to decrease atherogenic-dense LDL. Fibrates are typically orally administered to such patients. Fenofibrate or 2-[4-(4-chlorobenzoyl)phenoxy]-2-methyl-propanoic acid, 1-methylethyl ester, has been known for many years as a medicinally active principle because of its efficacy in lowering blood triglyceride and cholesterol levels.

In some embodiments, the present disclosure provides a method of treating or preventing age-related macular degeneration (AMD) by administering to a subject in need thereof an effective amount of an active compound or its salt or composition as described herein in combination with an anti-VEGF agent. Non-limiting examples of anti-VEGF agents include, but are not limited to, aflibercept (EYLEA®; Regeneron Pharmaceuticals); ranibizumab (LUCENTIS®: Genentech and Novartis); pegaptanib (MACUGEN®; OSI Pharmaceuticals and Pfizer); bevacizumab (Avastin; Genentech/Roche); lapatinib (TYKERB®); sunitinib (SUTENT®); axitinib (INLYTA®); pazopanib; sorafenib (NEXAVAR®); ponatinib (INCLUSIG®); regorafenib (STIVARGA®); Cabozantinib (Abometyx; COMETRIQ®); vendetanib (CAPRELSA®); ramucirumab (CYRAMZA®); lenvatinib (LENVIMA®); ziv-aflibercept (ZALTRAP®); cediranib (RECENTIN®); anecortane acetate, squalamine lactate, and corticosteroids, including, but not limited to, triamcinolone acetonide.

In some embodiments, the disclosure provides a method of treating or preventing age-related macular degeneration (AMD) by administering to a subject in need thereof an effective amount of an active compound or its salt or composition as described herein in combination with a complement C5 inhibitor, for example, a complement C5 inhibitor described herein and in the table above titled Non-limiting examples of potential therapeutics for combination therapy, including, but not limited to, eculizumab (Alexion Pharmaceuticals); ravulizumab (Alexion Pharmaceuticals); LFG316 (Novartis/Morphosys); cemdisiran, cemdisiran/ALN-CC5 (Alnylam); ARC1005 (Novo Nordisk); Coversin (Akari Therapeutics); Mubodine (Adienne Pharma); RA101348 (Ra Pharma); SOB1002 (Swedish Orphan Biovitrum); SOMAmers (SomaLogic); Erdigna (Adienne Pharma); ARC1905 (Ophthotech); MEDI7814 (Medlmmune); NOX-D19 (Noxxon); IFX-1, CaCP29 (InflaRx); PMX53, PMX205 (Cephalon, Teva); CCX168 (ChemoCentryx); ADC-1004 (Alligator Bioscience); and Anti-C5aR-151, NN8209; Anti-C5aR-215, NN8210 (Novo Nordisk); prozelimab (Regeneron); BCD-148 (Biocad); ABP-959 (Amgen); SB-12 (Samsung Bioepis Co., Ltd.); zilucoplan (Ra Pharma); and crovalimab (SKY59; Roche/Chugal).

In some embodiments, the present disclosure provides a method of treating or preventing age-related macular degeneration (AMD) by administering to a subject in need thereof an effective amount of an active compound or its salt or composition as described herein in combination with anti-properidin agent, for example, an anti-properidin agent as described above, including but not limited to NM9401 (Novelmed).

In some embodiments, the present disclosure provides a method of treating or preventing age-related macular degeneration (AMD) by administering to a subject in need thereof an effective amount of an active compound or its salt or composition as described herein in combination with a complement C3 inhibitor for example, a complement C3 inhibitor described above, including, but not limited to, a compstatin or compstatin analog, for example Compstatin/POT-4 (Potentia Pharmaceuticals); ARC1905 (Archemix); 4(1 MEW)APL-1, APL-2 (Apellis); CP40/AMY-101, PEG-Cp40 (Amyndas) Complement C3 or CAP C3 Convertase targeting molecules: TT30 (CR2/CFH) (Alexion); TT32 (CR2/CR1) (Alexion Pharmaceuticals); Nafamostat (FUT-175, Futhan) (Torri Pharmaceuticals); Bikaciomab, NM9308 (Novelmed); CVF, HC-1496 (InCode) ALXN1102/ALXN1103 (TT30) (Alexion Pharmaceuticals); rFH (Optherion); H17 C3 (C3b/iC3b) (EluSys Therapeutics); Mini-CFH (Amyndas) Mirococept (APT070); sCR1 (CDX-1135) (Celldex); and CRIg/CFH.

In some embodiments, the present disclosure provides a method of treating or preventing age-related macular degeneration (AMD) by administering to a subject in need thereof an effective amount of an active compound or its salt or composition as described herein in combination with an anti-factor H or anti-factor B agent selected from Anti-FB siRNA (Alnylam); FCFD4514S (Genentech/Roche) SOMAmers for CFB and CFD (SomaLogic); TA106 (Alexion Pharmaceuticals); 5C6, and AMY-301 (Amyndas).

In some embodiments, the present disclosure provides a method of treating or preventing age-related macular degeneration (AMD) by administering to a subject in need thereof an effective amount of an active compound or its salt or composition as described herein in combination with an anti-MASP2, anti-C1s or anti-CR3 molecules, for example, but not limited to: Cynryze (ViroPharma/Baxter); TNT003 (True North); OMS721 (Omeros); OMS906 (Omeros); and Imprime PGG (Biothera).

In some embodiments, the disclosure provides a method of treating or preventing age-related macular degeneration (AMD) by administering to a subject in need thereof an effective amount of an active compound or its salt or composition as described herein in combination with a multiple kinase inhibitor, for example as described herein including but not limited to Sorafenib Tosylate (NEXAVAR®); Imatinib Mesylate (GLEEVEC®); Sunitinib Malate (SUTENT®); Ponatinib (ICLUSIG®); Axitinib (INLYTA®); Nintedanib (OFEV®); Pazopanib HCl (VOTRIENT®); Dovitinib (TKI-258, Oncology Ventures); gilteritnib (XOSPATA®); Linifanib (ABT-869); Crenolanib (CP-868596); Masitinib (AB1010); Tivozanib (FOTIVDA®); Motesanib Diphosphate (AMG-706); Amuvatinib (MP-470); TSU-68 (SU6668, Orantinib); CP-673451; Ki8751; Telatinib (BAY 57-9352); PP121; KRN 633; MK-2461; Tyrphostin (AG 1296); Sennoside B; AZD2932; and Trapidil.

In some embodiments, the disclosure provides a method of treating or preventing cold agglutinin disease (CAD) by administering to a subject in need thereof an effective amount of an active compound or its salt or composition as described herein in combination with a complement C5 inhibitor, for example, a complement C5 inhibitor described herein and in the table above titled Non-limiting examples of potential therapeutics for combination therapy, including, but not limited to, eculizumab (Alexion Pharmaceuticals); ravulizumab (Alexion Pharmaceuticals); LFG316 (Novartis/Morphosys); cemdisiran, cemdisiran/ALN-CC5 (Alnylam); ARC1005 (Novo Nordisk); Coversin (Akari Therapeutics); Mubodine (Adienne Pharma); RA101348 (Ra Pharma); SOB1002 (Swedish Orphan Biovitrum); SOMAmers (SomaLogic); Erdigna (Adienne Pharma); ARC1905 (Ophthotech); MEDI7814 (Medlmmune); NOX-D19 (Noxxon); IFX-1, CaCP29 (InflaRx); PMX53, PMX205 (Cephalon, Teva); CCX168 (ChemoCentryx); ADC-1004 (Alligator Bioscience); and Anti-C5aR-151, NN8209; Anti-C5aR-215, NN8210 (Novo Nordisk); prozelimab (Regeneron); BCD-148 (Biocad); ABP-959 (Amgen); SB-12 (Samsung Bioepis Co., Ltd.); zilucoplan (Ra Pharma); and crovalimab (SKY59; Roche/Chugal).

In some embodiments, the disclosure provides a method of treating or preventing cold agglutinin disease (CAD) by administering to a subject in need thereof an effective amount of an active compound or its salt or composition as described herein in combination with anti-properdin agent, for example, an anti-properdin agent as described above, including but not limited to NM9401 (Novelmed).

In some embodiments, the disclosure provides a method of treating or preventing cold agglutinin disease (CAD) by administering to a subject in need thereof an effective amount of an active compound or its salt or composition as described herein in combination with a complement C3 inhibitor for example, a complement C3 inhibitor described above, including, but not limited to, a compstatin or compstatin analog, for example Compstatin/POT-4 (Potentia Pharmaceuticals); ARC1905 (Archemix); 4(1 MEW)APL-1, APL-2 (Apellis); CP40/AMY-101, PEG-Cp40 (Amyndas) Complement C3 or CAP C3 Convertase targeting molecules: TT30 (CR2/CFH) (Alexion); TT32 (CR2/CR1) (Alexion Pharmaceuticals); Nafamostat (FUT-175, Futhan) (Torri Pharmaceuticals); Bikaciomab, NM9308 (Novelmed); CVF, HC-1496 (InCode) ALXN1102/ALXN1103 (TT30) (Alexion Pharmaceuticals); rFH (Optherion); H17 C3 (C3b/iC3b) (EluSys Therapeutics); Mini-CFH (Amyndas) Mirococept (APT070); sCR1 (CDX-1135) (Celldex); and CRIg/CFH.

In some embodiments, the disclosure provides a method of treating or preventing cold agglutinin disease (CAD) by administering to a subject in need thereof an effective amount of an active compound or its salt or composition as described herein in combination with an anti-factor H or anti-factor B agent selected from IONIS-FB-LRx (lonis Pharmaceuticals); Anti-FB siRNA (Alnylam); FCFD4514S (Genentech/Roche) SOMAmers for CFB and CFD (SomaLogic); TA106 (Alexion Pharmaceuticals); 5C6, and AMY-301 (Amyndas).

In some embodiments, the disclosure provides a method of treating or preventing cold agglutinin disease (CAD) by administering to a subject in need thereof an effective amount of an active compound or its salt or composition as described herein in combination with an anti-MASP2, anti C1s, or anti-C1n molecule, for example but not limited to Cinryze® (Takeda); Berinert® (Bering CSL), Ruconest® (Pharming), Haegarda® (Bering CSL); TNT003 (Bioverativ/Sanofi); BIVV009 (Bioverativ/Sanofi); BIVV020 (Bioverativ/Sanofi); OMS721 (Omeros); OMS906 (Omeros); and Imprime PGG (Biothera)

In some embodiments, the disclosure provides a method of treating or preventing cold agglutinin disease (CAD) by administering to a subject in need thereof an effective amount of an active compound or its salt or composition as described herein in combination with a multiple kinase inhibitor, for example as described herein including but not limited to Sorafenib Tosylate (NEXAVAR®); Imatinib Mesylate (GLEEVEC®); Sunitinib Malate (SUTENT®); Ponatinib (ICLUSIG®); Axitinib (INLYTA®); Nintedanib (OFEV®); Pazopanib HCl (VOTRIENT®); Dovitinib (TKI-258, Oncology Ventures); gilteritnib (XOSPATA®); Linifanib (ABT-869); Crenolanib (CP-868596); Masitinib (AB1010); Tivozanib (FOTIVDA®); Motesanib Diphosphate (AMG-706); Amuvatinib (MP-470); TSU-68 (SU6668, Orantinib); CP-673451; Ki8751; Telatinib (BAY 57-9352); PP121; KRN 633; MK-2461; Tyrphostin (AG 1296); Sennoside B; AZD2932; and Trapidil.

In some embodiments, the present disclosure provides a method of treating or preventing paroxysmal nocturnal hemoglobinuria (PNH) by administering to a subject in need thereof an effective amount of an active compound or its salt or composition as described herein with an additional inhibitor of the complement system or another active compound with a different biological mechanism of action.

In another embodiment, the present disclosure provides a method of treating or preventing paroxysmal nocturnal hemoglobinuria (PNH) by administering to a subject in need thereof an effective amount of an active compound or its salt or composition as described herein in combination or alternation with eculizumab or ravulizumab.

In another embodiment, the present disclosure provides a method of treating or preventing paroxysmal nocturnal hemoglobinuria (PNH) by administering to a subject in need thereof an effective amount of an active compound or its salt or composition as described herein in combination or alternation with CP40.

In some embodiments, the additional agent is PEGylated-CP40. CP40 is a peptide inhibitor that shows a strong binding affinity for C3b and inhibits hemolysis of paroxysmal nocturnal hemoglobinuria (PNH) erythrocytes. In some embodiments, the additional agent is a complement component inhibitor, for example but not limited to Compstatin/POT-4 (Potentia Pharmaceuticals); ARC1905 (Archemix); 4(1 MEW)APL-1, APL-2 (Apellis); CP40/AMY-101, PEG-Cp40 (Amyndas); a PDGF inhibitor, for example, but not limited to Sorafenib Tosylate; Imatinib Mesylate (ST1571); Sunitinib Malate; Ponatinib (AP24534); Axitinib; Imatinib (ST1571); Nintedanib (BIBF 1120); Pazopanib HCl (GW786034 HCl); Dovitinib (TKI-258, CHIR-258); Linifanib (ABT-869); Crenolanib (CP-868596); Masitinib (AB1010); Tivozanib (AV-951); Motesanib Diphosphate (AMG-706); Amuvatinib (MP-470); TSU-68 (SU6668, Orantinib); CP-673451; Ki8751; Telatinib; PP121; Pazopanib; KRN 633; Dovitinib (TKI-258) Dilactic Acid; MK-2461; Tyrphostin (AG 1296); Dovitinib (TK1258) Lactate; Sennoside B; Sunitinib; AZD2932; and Trapidil; an anti-factor H or anti-factor B agent, for example anti-FB siRNA (Alnylam); FCFD4514S (Genentech/Roche) SOMAmers for CFB and CFD (SomaLogic); TA106 (Alexion Pharmaceuticals); 5C6, and AMY-301 (Amyndas); a complement C3 or CAP C3 convertase targeting molecule, for example but not limited to TT30 (CR2/CFH) (Alexion); TT32 (CR2/CR1) (Alexion Pharmaceuticals); Nafamostat (FUT-175, Futhan) (Torri Pharmaceuticals); Bikaciomab, NM9308 (Novelmed); CVF, HC-1496 (InCode) ALXN1102/ALXN1103 (TT30) (Alexion Pharmaceuticals); rFH (Optherion); H17 C3 (C3b/iC3b) (EluSys Therapeutics); Mini-CFH (Amyndas) Mirococept (APT070); sCR1 (CDX-1135) (Celldex); CRIg/CFH, an anti-CR3, anti-MASP2, anti C1s, or anti-C1n molecule, for example but not limited to Cinryze (Takeda); TNT003 (True North); OMS721 (Omeros); OMS906 (Omeros); and Imprime PGG (Biothera)

In some embodiments, the present disclosure provides a method of treating or preventing rheumatoid arthritis by administering to a subject in need thereof an effective amount of a composition comprising an active compound or its salt or composition as described herein in combination or alternation with an additional inhibitor of the complement system, or an active agent that functions through a different mechanism of action.

In another embodiment, the present disclosure provides a method of treating or preventing rheumatoid arthritis by administering to a subject in need thereof an effective amount of an active compound or its salt or composition as described herein in combination or alternation with methotrexate.

In certain embodiments, an active compound or its salt or composition as described herein is administered in combination or alternation with at least one additional therapeutic agent selected from: salicylates including aspirin (ANACIN®, ASCRIPTIN®, BAYER ASPIRIN®, ECOTRIN®) and salsalate (MONO-GESIC®, SALGESIC®); nonsteroidal anti-inflammatory drugs (NSAIDs); nonselective inhibitors of the cyclo-oxygenase (COX-1 and COX-2) enzymes, including diclofenac (CATAFLAM®, VOLTAREN®), ibuprofen (ADVIL®, MOTRIN®), ketoprofen (ORUDIS®), naproxen (ALEVE®, NAPROSYN*), piroxicam (Feldene), etodolac (LODINE®), indomethacin, oxaprozin (DAYPRO®), nabumetone (RELAFEN®), and meloxicam (MOBIC®); selective cyclo-oxygenase-2 (COX-2) inhibitors including Celecoxib (CELEBREX®); disease-modifying antirheumatic drugs (DMARDs), including azathioprine (IMURAN®), cyclosporine (Sandimmune, NEORAL®), gold salts (RIDAURA®, SOLGANAL®, AUROLATE®, MYOCHRYSINE®), hydroxychloroquine (PLAQUENIL®), leflunomide (ARAVA®), methotrexate (RHEUMATREX*), penicillamine (CUPRIMINE®), and sulfasalazine (AZULFIDINE®); biologic drugs including abatacept (ORENCIA®), etanercept (ENBREL®), infliximab (REMICADE®), adalimumab (HUMIRA®), and anakinra (KINERET®); corticosteroids including betamethasone (CELESTONE® SOLUSPAN®), cortisone (CORTONE®), dexamethasone (DECADRON®), methylprednisolone (SOLUMEDROL®, DEPOMEDROL®), prednisolone (DELTA-CORTEF®), prednisone (DELTASONE®, ORASONE®), and triamcinolone (Aristocort); gold salts, including Auranofin (RIDAURA®); Aurothioglucose (SOLGANAL®); Aurolate; Myochrysine; or any combination thereof.

In some embodiments, the present disclosure provides a method of treating or preventing multiple sclerosis by administering to a subject in need thereof an effective amount of an active compound or its salt or composition as described herein in combination or alternation with an additional inhibitor of the complement system, or an active agent that functions through a different mechanism of action.

In another embodiment, the present disclosure provides a method of treating or preventing multiple sclerosis by administering to a subject in need thereof an effective amount of an active compound or its salt or composition as described herein in combination or alternation with a corticosteroid.

Examples of corticosteroids include, but are not limited to, prednisone, dexamethasone, solumedrol, and methylprednisolone. In some embodiments, an active compound or its salt or composition as described herein is combined with at least one anti-multiple sclerosis drug, for example, selected from: AUBAGIO® (teriflunomide), AVONEX® (interferon beta-1a), BETASERON® (interferon beta-1 b), COPAXONE® (glatiramer acetate), EXTAVIA® (interferon beta-1 b), GILENYA® (fingolimod), LEMTRADA® (alemtuzumab), Novantrone (mitoxantrone), PLEGRIDY® (peginterferon beta-1a), REBIF® (interferon beta-1a), TECFIDERA® (dimethyl fumarate), TYSABRI® (natalizumab), SOLUMEDROL® (methylprednisolone), High-dose oral DELTASONE® (prednisone), H. P. ACTHAR GEL® (ACTH), or a combination thereof.

In some embodiments, an active compound or its salt or composition as described herein is useful in a combination with another pharmaceutical agent to ameliorate or reduce a side effect of the agent. For example, in some embodiments, an active compound or its salt or composition as described herein may be used in combination with adoptive cell transfer therapies to reduce an associated inflammatory response associated with such therapies, for example, a cytokine mediated response such as cytokine release syndrome. In some embodiments, the adoptive cell transfer therapy includes the use of a chimeric antigen receptor T-Cell (CAR T). In some embodiments, the adoptive cell transfer therapy includes the use of a chimeric antigen receptor T-Cell (CAR T) or a dendritic cell to treat a hematologic or solid tumor, for example, a B-cell related hematologic cancer. In some embodiments, the hematologic or solid tumor is acute lymphoblastic leukemia (ALL), acute myeloid leukemia (AML), non-Hodgkin's lymphoma, chronic lymphocytic leukemia (CLL), pancreatic cancer, glioblastoma, or a cancer that expresses CD19.

In an additional alternative embodiment, an active compound or its salt or composition as described herein may be provided in combination with eculizumab or ravulizumab for the treatment of PNH, aHUSs, STEC-HUS, ANCA-vasculitis, AMD, CAD, C3 glomerulopathy, for example DDD or C3GN, chronic hemolysis, neuromyelitis optica, or transplantation rejection. In some embodiments, an active compound or its salt or composition as described herein may be provided in combination with compstatin or a compstatin derivative for the treatment of PNH, aHUSs, STEC-HUS, ANCA-vasculitis, AMD, CAD, C3 glomerulopathy, for example DDD or C3GN, chronic hemolysis, neuromyelitis optica, neuromyelitis optica spectrum disorder in adults who are anti-aquaporin-4 (AQP4) antibody positive, myasthenia gravis, generalized myasthenia gravis, or transplantation rejection. In some embodiments, the additional agent is a complement component inhibitor, for example but not limited to Compstatin/POT-4 (Potentia Pharmaceuticals); ARC1905 (Archemix); 4(1 MEW)APL-1, APL-2 (Apellis); CP40/AMY-101, PEG-Cp40 (Amyndas); a PDGF inhibitor, for example, but not limited to Sorafenib Tosylate; Imatinib Mesylate (ST1571); Sunitinib Malate; Ponatinib (AP24534); Axitinib; Imatinib (ST1571); Nintedanib (BIBF 1120); Pazopanib HCl (GW786034 HCl); Dovitinib (TKI-258, CHIR-258); Linifanib (ABT-869); Crenolanib (CP-868596); Masitinib (AB1010); Tivozanib (AV-951); Motesanib Diphosphate (AMG-706); Amuvatinib (MP-470); TSU-68 (SU6668, Orantinib); CP-673451; Ki8751; Telatinib; PP121; Pazopanib; KRN 633; Dovitinib (TKI-258) Dilactic Acid; MK-2461; Tyrphostin (AG 1296); Dovitinib (TK1258) Lactate; Sennoside B; Sunitinib; AZD2932; and Trapidil; an anti-factor H or anti-factor B agent, for example anti-FB siRNA (Alnylam); FCFD4514S (Genentech/Roche) SOMAmers for CFB and CFD (SomaLogic); TA106 (Alexion Pharmaceuticals); 5C6, and AMY-301 (Amyndas); a complement C3 or CAP C3 convertase targeting molecule, for example but not limited to TT30 (CR2/CFH) (Alexion); TT32 (CR2/CR1) (Alexion Pharmaceuticals); Nafamostat (FUT-175, Futhan) (Torri Pharmaceuticals); Bikaciomab, NM9308 (Novelmed); CVF, HC-1496 (InCode) ALXN1102/ALXN1103 (TT30) (Alexion Pharmaceuticals); rFH (Optherion); H17 C3 (C3b/iC3b) (EluSys Therapeutics); Mini-CFH (Amyndas) Mirococept (APT070); sCR1 (CDX-1135) (Celldex); CRIg/CFH, an anti-CR3, anti-MASP2, anti C1s, or anti-C1 n molecule, for example but not limited to Cinryze (Takeda); TNT003 (True North); OMS721 (Omeros); OMS906 (Omeros); and Imprime PGG (Biothera).

In some embodiments, an active compound or its salt or composition as described herein may be provided in combination with rituxan for the treatment of a complement mediated disorder. In some embodiments, the complement mediated disorder is, for example, rheumatoid arthritis, Granulomatosis with Polyangiitis (GPA) (Wegener's Granulomatosis), and Microscopic Polyangiitis (MPA). In some embodiments, the disorder is Lupus.

In some embodiments, an active compound or its salt or composition as described herein may be provided in combination with cyclophosphamide for the treatment of a complement mediated disorder. In some embodiments, the disorder is an autoimmune disease. In some embodiments, the complement mediated disorder is, for example, rheumatoid arthritis, Granulomatosis with Polyangiitis (GPA) (Wegener's Granulomatosis), and Microscopic Polyangiitis (MPA). In some embodiments, the disorder is Lupus.

In some embodiments, an active compound or its salt or composition as described herein is dosed in combination with a conventional DLE treatment for the treatment of lupus to a subject in need thereof.

Examples of conventional DLE treatments include topical corticosteroid ointments or creams, such as triamcinolone acetonide, fluocinolone, flurandrenolide, betamethasone valerate, or betamethasone dipropionate. Resistant plaques can be injected with an intradermal corticosteroid. Other potential DLE treatments include calcineurin inhibitors such as pimecrolimus cream or tacrolimus ointment. Particularly resistant cases can be treated with systemic antimalarial drugs, such as hydroxychloroquine (PLAQUENIL).

In some embodiments, an active compound or its salt or composition as described herein may be provided in combination with methotrexate for the treatment of Lupus.

In some embodiments, an active compound or its salt or composition as described herein may be provided in combination with azathioprine for the treatment of Lupus.

In some embodiments, an active compound or its salt or composition as described herein may be provided in combination with a non-steroidal anti-inflammatory drug for the treatment of Lupus.

In some embodiments, an active compound or its salt or composition as described herein may be provided in combination with a corticosteroid for the treatment of Lupus.

In some embodiments, an active compound or its salt or composition as described herein may be provided in combination with a belimumab (Benlysta) for the treatment of Lupus.

In some embodiments, an active compound or its salt or composition as described herein may be provided in combination with hydroxychloroquine (Plaquenil) for the treatment of Lupus.

In some embodiments, an active compound or its salt or composition as described herein may be provided in combination with sifalimumab for the treatment of Lupus.

In some embodiments, an active compound or its salt or composition as described herein may be provided in combination with OMS721 (Omeros) for the treatment of a complement mediated disorder. In some embodiments, an active compound or its salt or composition as described herein may be provided in combination with OMS906 (Omeros) for the treatment of a complement mediated disorder. In some embodiments, the complement mediated disorder is, for example, thrombotic thrombocytopenic purpura (TTP) or aHUS.

In some embodiments, an active compound or its salt or composition as described herein may be provided in combination with an anti-inflammatory agent, immunosuppressive agent, or anti-cytokine agent for the treatment or prevention of cytokine or inflammatory reactions in response to the administration of pharmaceuticals or biotherapeutics (e.g. adoptive T-cell therapy (ACT) such as CAR T-cell therapy, or monoclonal antibody therapy).

In some embodiments, an active compound or its salt or composition as described herein may be provided in combination with a corticosteroid, for example prednisone, dexamethasone, solumedrol, and methylprednisolone, and/or anti-cytokine compounds targeting, e.g., IL-4, IL-10, IL-11, IL-13 and TGFβ.

In some embodiments, an active compound or its salt or composition as described herein may be provided in combination with an anti-cytokine inhibitor including, but are not limited to, adalimumab, infliximab, etanercept, protopic, efalizumab, alefacept, anakinra, siltuximab, secukibumab, ustekinumab, golimumab, and tocilizumab, or a combination thereof.

Additional anti-inflammatory agents that can be used in combination with an active compound or its salt or composition as described herein include, but are not limited to, non-steroidal anti-inflammatory drug(s) (NSAIDs); cytokine suppressive anti-inflammatory drug(s) (CSAIDs); CDP-571/BAY-10-3356 (humanized anti-TNFα antibody; Celltech/Bayer); cA2/infliximab (chimeric anti-TNFα antibody; Centocor); 75 kdTNFR-IgG/etanercept (75 kD TNF receptor-IgG fusion protein; Immunex); 55 kdTNF-IgG (55 kD TNF receptor-IgG fusion protein; Hoffmann-LaRoche); IDEC-CE9.1/SB 210396 (non-depleting primatized anti-CD4 antibody; IDEC/SmithKline); DAB 486-IL-2 and/or DAB 389-IL-2 (IL-2 fusion proteins; Seragen); Anti-Tac (humanized anti-IL-2Ra; Protein Design Labs/Roche); IL-4 (anti-inflammatory cytokine; DNAX/Schering); IL-10 (SCH 52000; recombinant IL-10, anti-inflammatory cytokine; DNAX/Schering); IL-4; IL-10 and/or IL-4 agonists (e.g., agonist antibodies); IL-1RA (IL-1 receptor antagonist; Synergen/Amgen); anakinra (Kineret®/Amgen); TNF-bp/s-TNF (soluble TNF binding protein); R⁹⁷³⁴⁰¹ (phosphodiesterase Type IV inhibitor); MK-966 (COX-2 Inhibitor); Iloprost, leflunomide (anti-inflammatory and cytokine inhibiton); tranexamic acid (inhibitor of plasminogen activation); T-614 (cytokine inhibitor); prostaglandin E1; Tenidap (non-steroidal anti-inflammatory drug); Naproxen (non-steroidal anti-inflammatory drug); Meloxicam (non-steroidal anti-inflammatory drug); Ibuprofen (non-steroidal anti-inflammatory drug); Piroxicam (non-steroidal anti-inflammatory drug); Diclofenac (non-steroidal anti-inflammatory drug); Indomethacin (non-steroidal anti-inflammatory drug); Sulfasalazine; Azathioprine; ICE inhibitor (inhibitor of the enzyme interleukin-1p converting enzyme); zap-70 and/or Ick inhibitor (inhibitor of the tyrosine kinase zap-70 or Ick); TNF-convertase inhibitors; anti-IL-12 antibodies; anti-IL-18 antibodies; interleukin-11; interleukin-13; interleukin-17 inhibitors; gold; penicillamine; chloroquine; chlorambucil; hydroxychloroquine; cyclosporine; cyclophosphamide; antithymocyte globulin; anti-CD4 antibodies; CD5-toxins; orally-administered peptides and collagen; lobenzarit disodium; Cytokine Regulating Agents (CRAB) HP228 and HP466 (Houghten Pharmaceuticals, Inc.); ICAM-1 antisense phosphorothioate oligo-deoxynucleotides (ISIS 2302; Isis Pharmaceuticals, Inc.); soluble complement receptor 1 (TP10; T Cell Sciences, Inc.); prednisone; orgotein; glycosaminoglycan polysulphate; minocycline; anti-IL2R antibodies; marine and botanical lipids (fish and plant seed fatty acids); auranofin; phenylbutazone; meclofenamic acid; flufenamic acid; intravenous immune globulin; zileuton; azaribine; mycophenolic acid (RS-61443); tacrolimus (FK-506); sirolimus (rapamycin); amiprilose (therafectin); cladribine (2-chlorodeoxyadenosine).

In a specific embodiment, an active compound or its salt or composition as described herein may be provided in combination with a corticosteroid for the treatment or prevention of cytokine or inflammatory reactions in response to the administration of pharmaceuticals or biotherapeutics.

In another embodiment, an active compound or its salt or composition as described herein may be provided in combination with etarnercept for the treatment or prevention of cytokine or inflammatory reactions in response to the administration of pharmaceuticals or biotherapeutics.

In another embodiment, an active compound or its salt or composition as described herein may be provided in combination with tocilizumab for the treatment or prevention of cytokine or inflammatory reactions in response to the administration of pharmaceuticals or biotherapeutics.

In another embodiment, an active compound or its salt or composition as described herein may be provided in combination with etarnercept and tocilizumab for the treatment or prevention of cytokine or inflammatory reactions in response to the administration of pharmaceuticals or biotherapeutics.

In another embodiment, an active compound or its salt or composition as described herein may be provided in combination with infliximab for the treatment or prevention of cytokine or inflammatory reactions in response to the administration of pharmaceuticals or biotherapeutics.

In another embodiment, an active compound or its salt or composition as described herein may be provided in combination with golimumab for the treatment or prevention of cytokine or inflammatory reactions in response to the administration of pharmaceuticals or biotherapeutics.

In a specific embodiment, an active compound or its salt or composition as described herein may be provided in combination with methylprednisolone, azathioprine, mycophenolate, rituximab, methotrexate, an oral corticosteroid, mitoxantrone, tocilizumab, or a C5 inhibitor such as eculizumab or ravulizumab, or a combination thereof, for the treatment of NMO.

In a specific embodiment, an active compound or its salt or composition as described herein may be provided in combination with Carbidopa-levodopa, a Dopamine agonists including, but not limited to pramipexole (Mirapex), ropinirole (Requip) and rotigotine (Neupro, given as a patch). Apomorphine (Apokyn), an MAO B inhibitors, for example selegiline (Eldepryl, Zelapar), rasagiline (Azilect) and safinamide (Xadago), a Catechol O-methyltransferase (COMT) inhibitor, for example Entacapone (Comtan) and Tolcapone (Tasmar), an Anticholinergics., for example benztropine (Cogentin) or trihexyphenidyl, or Amantadine, or a combination thereof, for the treatment of Parkinson's Disease.

In a specific embodiment, an active compound or its salt or composition as described herein may be provided in combination with a cholinesterase inhibitor, Namenda, risperidone (Risperdal), olanzapine (Zyprexa), and quetiapine (Seroquel), vitamin E, sertraline (Zoloft), bupropion (Wellbutrin), citalopram (Celexa), paroxetine (Paxil), or venlafaxine (Effexor), or a combination thereof, for the treatment of Alzheimer's Disease.

In a specific embodiment, an active compound or its salt or composition as described herein may be provided in combination with Riluzole (Rilutek), Edaravone (Radicava), or a combination thereof, for the treatment of ALS.

In one aspect, an active compound or its salt or composition as described herein may be provided in combination with an immune modulator for the treatment of cancer, including but not limited to a checkpoint inhibitor, including as non-limiting examples, a PD-1 inhibitor, PD-L1 inhibitor, PD-L2 inhibitor, CTLA-4 inhibitor, LAG-3 inhibitor, TIM-3 inhibitor, V-domain Ig suppressor of T-cell activation (VISTA) inhibitors, small molecule, peptide, nucleotide, or other inhibitor. In certain aspects, the immune modulator is an antibody, such as a monoclonal antibody.

Immune checkpoint inhibitors for use in the methods described herein include, but are not limited to PD-1 inhibitors, PD-L1 inhibitors, PD-L2 inhibitors, CTLA-4 inhibitors, LAG-3 inhibitors, TIM-3 inhibitors, and V-domain Ig suppressor of T-cell activation (VISTA) inhibitors, or combinations thereof.

In some embodiments, the immune checkpoint inhibitor is a PD-1 inhibitor that blocks the interaction of PD-1 and PD-L1 by binding to the PD-1 receptor, and in turn inhibits immune suppression.

In some embodiments, the immune checkpoint inhibitor is a PD-1 immune checkpoint inhibitor selected from nivolumab (Opdivo®), pembrolizumab (Keytruda®), pidilizumab, AMP-224 (AstraZeneca and Medlmmune), PF-06801591 (Pfizer), MED10680 (AstraZeneca), PDR001 (Novartis), REGN2810 (Regeneron), MGA012 (MacroGenics), BGB-A317 (BeiGene) SHR-12-1 (Jiangsu Hengrui Medicine Company and Incyte Corporation), TSR-042 (Tesaro), and the PD-L1/VISTA inhibitor CA-170 (Curis Inc.).

In some embodiments, the immune checkpoint inhibitor is the PD-1 immune checkpoint inhibitor nivolumab (Opdivo®) administered in an effective amount for the treatment of Hodgkin lymphoma, melanoma, non-small cell lung cancer, hepatocellular carcinoma, or ovarian cancer. Nivolumab has been approved by the FDA for the use of metastatic melanoma, non-small cell lung cancer, and renal cell carcinoma.

In another aspect of this embodiment, the immune checkpoint inhibitor is the PD-1 immune checkpoint inhibitor pembrolizumab (Keytruda®) administered in an effective amount for the treatment of melanoma, non-small cell lung cancer, small cell lung cancer, head and neck cancer, or urothelial cancer.

In an additional aspect of this embodiment, the immune checkpoint inhibitor is the PD-1 immune checkpoint inhibitor pidilizumab (Medivation) administered in an effective amount for refractory diffuse large B-cell lymphoma (DLBCL) or metastatic melanoma.

In some embodiments, the immune checkpoint inhibitor is a PD-L1 inhibitor that blocks the interaction of PD-1 and PD-L1 by binding to the PD-L1 receptor, and in turn inhibits immune suppression. PD-L1 inhibitors include, but are not limited to, atezolizumab, durvalumab, KN035CA-170 (Curis Inc.), and LY3300054 (Eli Lilly). In some embodiments, the PD-L1 inhibitor is atezolizumab. In some embodiments, the PD-L1 inhibitor blocks the interaction between PD-L1 and CD80 to inhibit immune suppression.

In some embodiments, the immune checkpoint inhibitor is the PD-L1 immune checkpoint inhibitor atezolizumab (Tecentriq®) administered in an effective amount for the treatment of metastatic bladder cancer, metastatic melanoma, metastatic non-small cell lung cancer, or metastatic renal cell carcinoma.

In another aspect of this embodiment, the immune checkpoint inhibitor is durvalumab (AstraZeneca and Medlmmune) administered in an effective amount for the treatment of non-small cell lung cancer or bladder cancer.

In yet another aspect of the embodiment, the immune checkpoint inhibitor is KN035 (Alphamab) administered in an effective amount for the treatment of PD-L1 positive solid tumors. An additional example of a PD-L1 immune checkpoint inhibitor is BMS-936559 (Bristol-Myers Squibb), although clinical trials with this inhibitor have been suspended as of 2015.

In one aspect, the immune checkpoint inhibitor is a CTLA-4 immune checkpoint inhibitor that binds to CTLA-4 and inhibits immune suppression. CTLA-4 inhibitors include, but are not limited to, ipilimumab, tremelimumab (AstraZeneca and Medlmmune), AGEN1884 and AGEN2041 (Agenus).

In some embodiments, the CTLA-4 immune checkpoint inhibitor is ipilimumab (Yervoy®) administered in an effective amount for the treatment of metastatic melanoma, adjuvant melanoma, or non-small cell lung cancer.

In another embodiment, the immune checkpoint inhibitor is a LAG-3 immune checkpoint inhibitor. Examples of LAG-3 immune checkpoint inhibitors include, but are not limited to, BMS-986016 (Bristol-Myers Squibb), GSK2831781 (GlaxoSmithKline), IMP321 (Prima BioMed), LAG525 (Novartis), and the dual PD-1 and LAG-3 inhibitor MGDO13 (MacroGenics). In yet another aspect of this embodiment, the immune checkpoint inhibitor is a TIM-3 immune checkpoint inhibitor. A specific TIM-3 inhibitor includes, but is not limited to, TSR-022 (Tesaro).

Other immune checkpoint inhibitors for use in combination with the active compounds described herein for the treatment of cancer include, but are not limited to, B7-H₃/CD276 immune checkpoint inhibitors such as MGA217, indoleamine 2,3-dioxygenase (IDO) immune checkpoint inhibitors such as Indoximod and INCB024360, killer immunoglobulin-like receptors (KIRs) immune checkpoint inhibitors such as Lirilumab (BMS-986015), carcinoembryonic antigen cell adhesion molecule (CEACAM) inhibitors (e.g., CEACAM-1, -3 and/or -5). Exemplary anti-CEACAM-1 antibodies are described in WO 2010/125571, WO 2013/082366 and WO 2014/022332, e.g., a monoclonal antibody 34B1, 26H₇, and 5F4; or a recombinant form thereof, as described in, e.g., US 2004/0047858, U.S. Pat. No. 7,132,255 and WO 99/052552. In other embodiments, the anti-CEACAM antibody binds to CEACAM-5 as described in, e.g., Zheng et al. PLoS One. 2010 Sep. 2; 5(9). pii: e12529 (DOI:10:1371/journal.pone.0021146), or cross-reacts with CEACAM-1 and CEACAM-5 as described in, e.g., WO 2013/054331 and US 2014/0271618. Still other checkpoint inhibitors can be molecules directed to B and T lymphocyte attenuator molecule (BTLA), for example as described in Zhang et al., Monoclonal antibodies to B and T lymphocyte attenuator (BTLA) have no effect on in vitro B cell proliferation and act to inhibit in vitro T cell proliferation when presented in a cis, but not trans, format relative to the activating stimulus, Clin Exp Immunol. 2011 January; 163(1): 77-87.

As contemplated herein, the active compounds described herein, or a pharmaceutically acceptable salt thereof, is administered in an oral dosage form and can be in combination with any standard chemotherapeutic agent treatment modality for the treatment of cancer. In some embodiments the chemotherapeutic agent inhibits cell growth. In some embodiments, the chemotherapeutic agent administered is a DNA damaging chemotherapeutic agent. In some embodiments, the chemotherapeutic agent is a protein synthesis inhibitor, a DNA-damaging chemotherapeutic, an alkylating agent, a topoisomerase inhibitor, an RNA synthesis inhibitor, a DNA complex binder, a thiolate alkylating agent, a guanine alkylating agent, a tubulin binder, DNA polymerase inhibitor, an anticancer enzyme, RAC1 inhibitor, thymidylate synthase inhibitor, oxazophosphorine compound, integrin inhibitor such as cilengitide, camptothecin or homocamptothecin, antifolate or a folate antimetabolite.

In some embodiments, the additional therapeutic agent is trastuzumab. In some embodiments, the additional therapeutic agent is lapatinib.

In some embodiments, the additional therapeutic agent is osimertinib. In some embodiments, the additional therapeutic agent is alectinib.

In some embodiments, the additional therapeutic agent is a MEK inhibitor.

In some embodiments, the additional therapeutic agent is an Androgen Receptor ligand.

In some embodiments, the additional therapeutic agent is a BTK inhibitor.

In some embodiments, the additional therapeutic agents are a MEK inhibitor and a RAF inhibitor.

In some embodiments, the additional therapeutic agent is a RAF inhibitor. In some embodiments, the additional therapeutic agent is regorafenib.

In some embodiments, the MEK inhibitor is Binimetinib, Selumetinib, CI-040, PD-325901, PD035901, or TAK-733.

In another embodiment the MEK inhibitor is Tramatenib, U0126-EtOH, PD98059, Pimasertib, BIX 02188, AZD8330, PD318088, SL-327, Refametinib, Myricetin, BI-847325, Cobimetinib, APS-2-79 HCl, or GDC-0623.

In some embodiments, the RAF inhibitor is PLX-4720, Dabrafenib, GDC-0879, Lifrafenib, CCT196969, RAF265, AZ 628, NVP-BHG712, SB590885, ZM 336372, Sorafenib, GW5074, TAK-632, CEP-32496, Encorafenib, PLX7904, LY3009120, RO5126766, or MLN2480.

In some embodiments, the BTK inhibitor is CC-292, CNX-774, RN486, LFM-A13, ONO-4059, ibrutinib, Acalabrutinib, or CG1746.

In some embodiments, the Androgen Receptor ligand is MK-2866, Apalutamide, Andarine, Boldenone, testosterone enanthate, dihydrotestosterone, Galertone, dehydroepiandrosterone, cyproterone acetate, megestrol acetate, epiandrosterone, AZD3514, spironolactone, chloromadinone acetate, ODM-201, EPI-001.

In some embodiments, the EGFR inhibitor is Lapatinib, Afatinib, Neratinib, Catertinib, AG-490, CP-724714, Dacomitnib, WZ4002, Sapitinib, CUDC-101, AG-1478, PD153035 HCl, Pelitinib, AC480, AEE788, AP26113, OSI-420, WZ3146, WZ8040, AST-1306, Rociletinib, Genisten, Varlitinib, Icotinib, TAK-285, WHI-P154, Daphnetin, PD168393, Tyrphostin 9, CNX-2006, AG-18, Cetuximab, Nazartinib, NSC228155, AZ5104, Poziotnib, AZD3759, Lifirafenib, Olmutinib, Erlotinib, Naquotinib, EA1045, or CL-387785.

In some embodiments, an active compound described herein is combined with a DNA-damaging chemotherapeutic agent for the treatment of cancer. As used herein the term “DNA-damaging” chemotherapy or chemotherapeutic agent refers to treatment with a cytostatic or cytotoxic agent (i.e., a compound) to reduce or eliminate the growth or proliferation of undesirable cells, for example cancer cells, wherein the cytotoxic effect of the agent can be the result of one or more of nucleic acid intercalation or binding, DNA or RNA alkylation, inhibition of RNA or DNA synthesis, the inhibition of another nucleic acid-related activity (e.g., protein synthesis), or any other cytotoxic effect.

Such compounds include, but are not limited to, DNA damaging compounds that can kill cells. “DNA damaging” chemotherapeutic agents include, but are not limited to, alkylating agents, DNA intercalators, protein synthesis inhibitors, inhibitors of DNA or RNA synthesis, DNA base analogs, topoisomerase inhibitors, telomerase inhibitors, and telomeric DNA binding compounds.

For example, alkylating agents include alkyl sulfonates, such as busulfan, improsulfan, and piposulfan; aziridines, such as a benzodizepa, carboquone, meturedepa, and uredepa; ethylenimines and methylmelamines, such as altretamine, triethylenemelamine, triethylenephosphoramide, triethylenethiophosphoramide, and trimethylol melamine; nitrogen mustards such as chlorambucil, chlornaphazine, cyclophosphamide, estramustine, mechlorethamine, mechlorethamine oxide hydrochloride, melphalan, novembichine, phenesterine, prednimustine, trofosfamide, and uracil mustard; and nitroso ureas, such as carmustine, chlorozotocin, fotemustine, lomustine, nimustine, and ranimustine.

Other DNA-damaging chemotherapeutic agents include daunorubicin, doxorubicin, idarubicin, epirubicin, mitomycin, and streptozocin. Chemotherapeutic antimetabolites include gemcitabine, mercaptopurine, thioguanine, cladribine, fludarabine phosphate, fluorouracil (5-FU), floxuridine, cytarabine, pentostatin, methotrexate, azathioprine, acyclovir, adenine β-1-D-arabinoside, amethopterin, aminopterin, 2-aminopurine, aphidicolin, 8-azaguanine, azaserine, 6-azauracil, 2′-azido-2′-deoxynucleosides, 5-bromodeoxycytidine, cytosine β-1-D-arabinoside, diazooxynorleucine, dideoxynucleosides, 5-fluorodeoxycytidine, 5-fluorodeoxyuridine, and hydroxyurea.

Chemotherapeutic protein synthesis inhibitors that may be combined with the active compounds described herein include abrin, aurintricarboxylic acid, chloramphenicol, colicin E3, cycloheximide, diphtheria toxin, edeine A, emetine, erythromycin, ethionine, fluoride, 5-fluorotryptophan, fusidic acid, guanylyl methylene diphosphonate and guanylyl imidodiphosphate, kanamycin, kasugamycin, kirromycin, and O-methyl threonine. Additional protein synthesis inhibitors include modeccin, neomycin, norvaline, pactamycin, paromomycine, puromycin, ricin, shiga toxin, showdomycin, sparsomycin, spectinomycin, streptomycin, tetracycline, thiostrepton, and trimethoprim.

Inhibitors of DNA synthesis that may be combined with the active compounds described herein include alkylating agents such as dimethyl sulfate, nitrogen and sulfur mustards; intercalating agents, such as acridine dyes, actinomycins, anthracenes, benzopyrene, ethidium bromide, propidium diiodide-intertwining; and other agents, such as distamycin and netropsin. Topoisomerase inhibitors, such as irinotecan, teniposide, coumermycin, nalidixic acid, novobiocin, and oxolinic acid; inhibitors of cell division, including colcemide, mitoxantrone, colchicine, vinblastine, and vincristine; and RNA synthesis inhibitors including actinomycin D, α-amanitine and other fungal amatoxins, cordycepin (3′-deoxyadenosine), dichlororibofuranosyl benzimidazole, rifampicine, streptovaricin, and streptolydigin also can be used as the DNA damaging compound.

In some embodiments, the chemotherapeutic agent that may be combined with the active compounds described herein for the treatment of cancer is a DNA complex binder such as camptothecin, or etoposide; a thiolate alkylating agent such as nitrosourea, BCNU, CCNU, ACNU, or fotesmustine; a guanine alkylating agent such as temozolomide, a tubulin binder such as vinblastine, vincristine, vinorelbine, vinflunine, cryptophycin 52, halichondrins, such as halichondrin B, dolastatins, such as dolastatin 10 and dolastatin 15, hemiasterlins, such as hemiasterlin A and hemiasterlin B, colchicine, combrestatins, 2-methoxyestradiol, E7010, paclitaxel, docetaxel, epothilone, discodermolide; a DNA polymerase inhibitor such as cytarabine; an anticancer enzyme such as asparaginase; a Rac1 inhibitor such as 6-thioguanine; a thymidylate synthase inhibitor such as capecitabine or 5-FU; a oxazophosphorine compound such as Cytoxan; a integrin inhibitor such as cilengitide; an antifolate such as pralatrexate; a folate antimetabolite such as pemetrexed; or a camptothecin or homocamptothecin such as diflomotecan.

In some embodiments the topoisomerase inhibitor is a type I inhibitor. In another embodiment the topoisomerase inhibitor is a type II inhibitor.

Other DNA-damaging chemotherapeutic agents that may be combined with the active compounds described herein for the treatment of cancer include, but are not limited to, cisplatin, hydrogen peroxide, carboplatin, procarbazine, ifosfamide, bleomycin, plicamycin, taxol, transplatinum, thiotepa, oxaliplatin, and the like, and similar acting-type agents. In some embodiments, the DNA damaging chemotherapeutic agent is selected from the group consisting of cisplatin, carboplatin, camptothecin, and etoposide.

Other suitable chemotherapeutic agents that may be combined with the active compounds described herein include, but are not limited to, radioactive molecules, toxins, also referred to as cytotoxins or cytotoxic agents, which includes any agent that is detrimental to the viability of cells, agents, and liposomes or other vesicles containing chemotherapeutic compounds. General anticancer pharmaceutical agents include: Vincristine (Oncovin), liposomal vincristine (Marqibo®), Cytarabine (cytosine arabinoside, ara-C, or Cytosar®), L-asparaginase (Elspar) or PEG-L-asparaginase (pegaspargase or Oncaspar), Etoposide (VP-16), Teniposide (Vumon*), 6-mercaptopurine (6-MP or Purinethol®), Prednisone, and Dexamethasone (Decadron). Examples of additional suitable chemotherapeutic agents include but are not limited to 5-fluorouracil, dacarbazine, alkylating agents, anthramycin (AMC)), anti-mitotic agents, cis-dichlorodiamine platinum (II) (DDP) cisplatin), diamino dichloro platinum, anthracyclines, antibiotics, antimetabolites, asparaginase, BCG live (intravesical), bleomycin sulfate, calicheamicin, cytochalasin B, dactinomycin (formerly actinomycin), daunorubicin HCl, daunorubicin citrate, denileukin diftitox, dihydroxy anthracin dione, Docetaxel, doxorubicin HCl, E. coli L-asparaginase, Erwinia L-asparaginase, etoposide citrovorum factor, etoposide phosphate, gemcitabine HCl, idarubicin HCl, interferon α-2b, irinotecan HCl, maytansinoid, mechlorethamine HCl, melphalan HCl, mithramycin, mitomycin C, mitotane, polifeprosan 20 with carmustine implant, procarbazine HCl, streptozotocin, teniposide, thiotepa, topotecan HCl, valrubicin, vinblastine sulfate, vincristine sulfate, and vinorelbine tartrate.

Additional cytotoxic chemotherapeutic agents for use with the present disclosure include: epirubicin, abraxane, taxotere, epothilone, tafluposide, vismodegib, azacytidine, doxifluridine, vindesine, and vinorelbine.

In some embodiments, the chemotherapeutic agent that may be combined with the active compounds described herein for the treatment of cancer is a DNA complex binder. In some embodiments, the chemotherapeutic agent is a tubulin binder. In some embodiments, the chemotherapeutic agent is an alkylating agent. In some embodiments, the chemotherapeutic agent is a thiolate alkylating agent.

Additional chemotherapeutic agents that may be combined with the active compounds described herein for the treatment of cancer may include 2-methoxyestradiol or 2ME2, finasunate, etaracizumab (MEDI-522), HLL1, huN901-DM1, atiprimod, saquinavir mesylate, ritonavir, nelfinavir mesylate, indinavir sulfate, plitidepsin, P276-00, tipifarnib, lenalidomide, thalidomide, pomalidomide, simvastatin, and celecoxib. Chemotherapeutic agents useful in the present disclosure include, but are not limited to, Trastuzumab (HERCEPTIN®), Pertuzumab (PERJETA™), Lapatinib (TYKERB®), Gefitinib (IRESSA®), Erlotinib (TARCEVA®), Cetuximab (ERBITUX®), Panitumumab (VECTIBIX®), Vandetanib (CAPRELSA®), Vemurafenib (ZELBORAF®), Vorinostat (ZOLINZA®), Romidepsin (ISTODAX®), Bexarotene (TARGRETIN®), Alitretinoin (Panretin®), Tretinoin (VESANOID®), Carfilzomib (Kyprolis™), Pralatrexate (FOLOTYN®), Bevacizumab (AVASTIN®), Ziv-aflibercept (ZALTRAP®), Sorafenib (NEXAVAR®), Sunitinib (SUTENT®), Pazopanib (VOTRIENT®), Regorafenib (STIVARGA®), and Cabozantinib (Cometriq™).

Additional chemotherapeutic agents that may be combined with the active compounds described herein for the treatment of cancer include, but are not limited to, a calcineurin inhibitor, e.g. a cyclosporin or an ascomycin, e.g. Cyclosporin A (Neoral®), FK506 (tacrolimus), pimecrolimus, a mTOR inhibitor, e.g. rapamycin or a derivative thereof, e.g. Sirolimus (Rapamune®), Everolimus (Certican®), temsirolimus, zotarolimus, biolimus-7, biolimus-9, a rapalog, e.g. ridaforolimus, campath 1H, a S1P receptor modulator, a dual mTORC1 and mTORC2 inhibitor, eg. Vistusertib (AZD2014), e.g. fingolimod or an analogue thereof, an anti IL-8 antibody, mycophenolic acid or a salt thereof, e.g. sodium salt, or a prodrug thereof, e.g. Mycophenolate Mofetil (CellCept®), OKT3 (Orthoclone OKT3*), Prednisone, ATGAM®, Thymoglobulin®, Brequinar Sodium, OKT4, T10B9.A-3A, 33B3.1, 15-deoxyspergualin, tresperimus, Leflunomide Arava®, anti-CD25, anti-IL2R, Basiliximab (Simulect®), Daclizumab (Zenapax®), mizoribine, dexamethasone, ISAtx-247, SDZ ASM 981 (pimecrolimus, Elidel®), Abatacept, belatacept, LFA3Ig, etanercept (sold as ENBREL® by ImmuneXcite), adalimumab (HUMIRA®), infliximab (REMICADE®), an anti-LFA-1 antibody, natalizumab (ANTEGREN®), Enlimomab, gavilimomab, Golimumab, antithymocyte immunoglobulin, siplizumab, Alefacept, efalizumab, Pentasa, mesalazine, asacol, codeine phosphate, benorylate, fenbufen, naprosyn, diclofenac, etodolac, indomethacin, dasatinib (SPRYCEL®) nilotinib (TASIGNA®), bosutinib (BOSULIF®), Imatinib mesylate (GLEEVEC®) and ponatinib (ICLUSIG™) amifostine, dolasetron mesylate, dronabinol, epoetin-α, etidronate, filgrastim, fluconazole, goserelin acetate, gramicidin D, granisetron, leucovorin calcium, lidocaine, Mesna, ondansetron HCl, pilocarpine HCl, porfimer sodium, vatalanib, 1-dehydrotestosterone, allopurinol sodium, Betamethasone, sodium phosphate and betamethasone acetate, calcium leucovorin, conjugated estrogens, Dexrazoxane, Dibromomannitol, esterified estrogens, estradiol, estramustine phosphate sodium, ethinyl estradiol, flutamide, folinic acid, glucocorticoids, leuprolide acetate, levamisole HCl, medroxyprogesterone acetate, megestrol acetate, methyltestosterone, nilutamide, octreotide acetate, pamidronate disodium, procaine, propranolol, testolactone, tetracaine, toremifene citrate, and sargramostim.

In some embodiments, the chemotherapeutic agent that may be combined with the active compounds described herein for the treatment of cancer is an estrogen receptor ligands such as tamoxifen, raloxifene, fulvestrant, anordrin, bazedoxifene, broparestriol, chlorotrianisene, clomiphene citrate, cyclofenil, lasofoxifene, ormeloxifene, or toremifene; an androgen receptor ligand such as bicalutamide, enzalutamide, apalutamide, cyproterone acetate, chlormadinone acetate, spironolactone, canrenone, drospirenone, ketoconazole, topilutamide, abiraterone acetate, or cimetidine; an aromatase inhibitor such as letrozole, anastrozole, or exemestane; an anti-inflammatory such as prednisone; an oxidase inhibitor such as allopurinol; an anticancer antibody; an anticancer monoclonal antibody; an antibody against CD40 such as lucatumumab or dacetuzumab; an antibody against CD20 such as rituximab; an antibody that binds CD52 such as alemtuzumab; an antibody that binds integrin such as volociximab or natalizumab; an antibody against interleukin-6 receptor such as tocilizumab; an interleukin-2 memetic such as aldesleukin; an antibody that targets IGF1 like figitumumab; an antibody that targets DR4 such as mapatumumab; an antibody that targets TRAIL-R² such as lexatumumab or dulanermin; a fusion protein such as atacicept; a B cell inhibitor such as atacicept; a proteasome inhibitor such as carfilzomib, bortezomib, or marizomib; a HSP90 inhibitor such as tanespimycin; a HDAC inhibitor such as vorinostat, belinostat or panobinostat; a MAPK ligand such as talmapimod; a PKC inhibitor such as enzastaurin; a HER2 receptor ligand such as trastuzumab, lapatinib, or pertuzumab; an EGFR inhibitor such as gefitinib, erlotinib, cetuximab, panitumumab, or vandetanib; a natural product such as romidepsin; a retinoid such as bexarotene, tretinoin, or alitretinoin; a receptor tyrosine kinase (RTK) inhibitor such as sunitinib, regorafenib, or pazopanib; or a VEGF inhibitor such as ziv-aflibercept, bevacizumab or dovitinib.

Additional chemotherapeutic agents that may be combined with the active compounds described herein for the treatment of cancer, particularly in the treatment of abnormal tissue of the female reproductive system such as breast, ovarian, endometrial, or uterine cancer include an estrogen inhibitor including but not limited to a SERM (selective estrogen receptor modulator), a SERD (selective estrogen receptor degrader), a complete estrogen receptor degrader, or another form of partial or complete estrogen antagonist. Partial anti-estrogens like raloxifene and tamoxifen retain some estrogen-like effects, including an estrogen-like stimulation of uterine growth, and also, in some cases, an estrogen-like action during breast cancer progression which actually stimulates tumor growth.

In contrast, fulvestrant, a complete anti-estrogen, is free of estrogen-like action on the uterus and is effective in tamoxifen-resistant tumors. Non-limiting examples of anti-estrogen compounds are provided in WO 2014/19176 assigned to Astra Zeneca, WO2013/090921, WO 2014/203129, WO 2014/203132, and US2013/0178445 assigned to Olema Pharmaceuticals, and U.S. Pat. Nos. 9,078,871, 8,853,423, and 8,703,810, as well as US 2015/0005286, WO 2014/205136, and WO 2014/205138.

Additional non-limiting examples of anti-estrogen compounds include: SERMS such as anordrin, bazedoxifene, broparestriol, clomiphene citrate, cyclofenil, lasofoxifene, ormeloxifene, raloxifene, tamoxifen, toremifene, and fulvestrant; aromatase inhibitors such as aminoglutethimide, testolactone, anastrozole, exemestane, fadrozole, formestane, and letrozole; and antigonadotropins such as leuprorelin, cetrorelix, allylestrenol, chloromadinone acetate, delmadinone acetate, dydrogesterone, medroxyprogesterone acetate, megestrol acetate, nomegestrol acetate, norethisterone acetate, progesterone, and spironolactone.

Additional chemotherapeutic agents that may be combined with the active compounds described herein for the treatment of cancer, particularly in the treatment of abnormal tissue of the male reproductive system such as prostate or testicular cancer, include, but are not limited to, an androgen (such as testosterone) inhibitor including but not limited to a selective androgen receptor modulator, a selective androgen receptor degrader, a complete androgen receptor degrader, or another form of partial or complete androgen antagonist.

In some embodiments, the prostate or testicular cancer is androgen-resistant. Non-limiting examples of anti-androgen compounds are provided in WO 2011/156518 and U.S. Pat. Nos. 8,455,534 and 8,299,112. Additional non-limiting examples of anti-androgen compounds include: chlormadinone acetate, spironolactone, canrenone, drospirenone, ketoconazole, topilutamide, abiraterone acetate, and cimetidine.

The chemotherapeutic agent that may be combined with the active compounds described herein for the treatment of cancer may include a kinase inhibitor, including but not limited to a phosphoinositide 3-kinase (PI3K) inhibitor, a Bruton's tyrosine kinase (BTK) inhibitor, or a spleen tyrosine kinase (Syk) inhibitor, or a combination thereof.

PI3k inhibitors are well known. Examples of PI3 kinase inhibitors include, but are not limited to, Wortmannin, demethoxyviridin, perifosine, idelalisib, pictilisib, Palomid 529, ZSTK474, PWT33597, CUDC-907, and AEZS-136, duvelisib, GS-9820, GDC-0032 (2-[4-[2-(2-Isopropyl-5-methyl-1,2,4-triazol-3-yl)-5,6-dihydroimidazo[1,2-d][1,4]benzoxazepin-9-yl]pyrazol-1-yl]-2-methylpropanamide), MLN-1117 ((2R)-1-Phenoxy-2-butanyl hydrogen (S)-methylphosphonate; or Methyl(oxo) {[(2R)-I-phenoxy-2-butanyl]oxy}phosphonium)), BYL-719 ((2S)-N1-[4-Methyl-5-[2-(2,2,2-trifluoro-1,1-dimethylethyl)-4-pyridinyl]-2-thiazolyl]-1,2-pyrrolidinedicarboxamide), GSK2126458 (2,4-Difluoro-N-{2-(methyloxy)-5-[4-(4-pyridazinyl)-6-quinolinyl]-3-pyridinyl}benzenesulfonamide), TGX-221 ((±)-7-Methyl-2-(morpholin-4-yl)-9-(I-phenylaminoethyl)-pyrido[1,2-a]-pyrimidin-4-one), GSK2636771 (2-Methyl-1-(2-methyl-3-(trifluoromethyl)benzyl)-6-morpholino-IH-benzo[d]imidazole-4-carboxylic acid dihydrochloride), KIN-193 ((R)-2-((I-(7-methyl-2-morpholino-4-oxo-4H-pyrido[1,2-a]pyrimidin-9-yl)ethyl)amino)benzoic acid), TGR-1202/RP5264, GS-9820 ((S)-1-(4-((2-(2-aminopyrimidin-5-yl)-7-methyl-4-mohydroxypropan-1-one), GS-1101 (5-fluoro-3-phenyl-2-([S)]-1-[9H-purin-6-ylamino]-propyl)-3H-quinazolin-4-one), AMG-319, GSK-2269557, SAR245409 (N-(4-(N-(3-((3,5-dimethoxyphenyl)amino)quinoxalin-2-yl)sulfamoyl)phenyl)-3-methoxy-4 methylbenzamide), BAY80-6946 (2-amino-N-(7-methoxy-8-(3-morpholinopropoxy)-2,3-dihydroimidazo[1,2-c]quinaz), AS 252424 (5-[I-[5-(4-Fluoro-2-hydroxy-phenyl)-furan-2-yl]-meth-(Z)-ylidene]-thiazolidine-2,4-dione), CZ 24832 (5-(2-amino-8-fluoro-[1,2,4]triazolo[1,5-a]pyridin-6-yl)-N-tert-butylpyridine-3-sulfonamide), buparlisib (5-[2,6-Di(4-morpholinyl)-4-pyrimidinyl]-4-(trifluoromethyl)-2-pyridinamine), GDC-0941 (2-(IH-Indazol-4-yl)-6-[[4-(methylsulfonyl)-1-piperazinyl]methyl]-4-(4-morpholinyl)thieno[3,2-d]pyrimidine), GDC-0980 ((S)-1-(4-((2-(2-aminopyrimidin-5-yl)-7-methyl-4-morpholinothieno[3,2-d]pyrimidin-6 yl)methyl)piperazin-1-yl)-2-hydroxypropan-1-one (also known as RG7422)), SF1126 ((8S,14S,17S)-14-(carboxymethyl)-8-(3-guanidinopropyl)-17-(hydroxymethyl)-3,6,9,12,15-pentaoxo-1-(4-(4-oxo-8-phenyl-4H-chromen-2-yl)morpholino-4-ium)-2-oxa-7,10,13,16-tetraazaoctadecan-18-oate), PF-05212384 (N-[4-[[4-(Dimethylamino)-1-piperidinyl]carbonyl]phenyl]-N′-[4-(4,6-di-4-morpholinyl-I,3,5-triazin-2-yl)phenyl]urea), LY3023414, BEZ235 (2-Methyl-2-{4-[3-methyl-2-oxo-8-(quinolin-3-yl)-2,3-dihydro-IH-imidazo[4,5-c]quinolin-1-yl]phenyl}propanenitrile), XL-765 (N-(3-(N-(3-(3,5-dimethoxyphenylamino)quinoxalin-2-yl)sulfamoyl)phenyl)-3-methoxy-4-methylbenzamide), and GSK1059615 (5-[[4-(4-Pyridinyl)-6-quinolinyl]methylene]-2,4-thiazolidenedione), PX886 ([(3aR,6E,9S,9aR,1 OR,11 aS)-6-[[bis(prop-2-enyl)amino]methylidene]-5-hydroxy-9-(methoxymethyl)-9a,11a-dimethyl-1,4,7-trioxo-2,3,3a,9,10,11-hexahydroindeno[4,5h]isochromen-10-yl]acetate (also known as sonolisib)), and the structure described in WO2014/071109.

BTK inhibitors are well known. Examples of BTK inhibitors include ibrutinib (also known as PCI-32765)(Imbruvica™) (1-[(3R)-3-[4-amino-3-(4-phenoxy-phenyl)pyrazolo[3,4-d]pyrimidin-1-yl]piperidin-1-yl]prop-2-en-1-one), dianilinopyrimidine-based inhibitors such as AVL-101 and AVL-291/292 (N-(3-((5-fluoro-2-((4-(2-methoxyethoxy)phenyl)amino)pyrimidin-4-yl)amino)phenyl)acrylamide) (Avila Therapeutics) (see US Patent Publication No 2011/0117073, incorporated herein in its entirety), dasatinib ([N-(2-chloro-6-methylphenyl)-2-(6-(4-(2-hydroxyethyl)piperazin-1-yl)-2-methylpyrimidin-4-ylamino)thiazole-5-carboxamide], LFM-A13 (alpha-cyano-beta-hydroxy-beta-methyl-N-(2,5-ibromophenyl) propenamide), GDC-0834 ([R—N-(3-(6-(4-(1,4-dimethyl-3-oxopiperazin-2-yl)phenylamino)-4-methyl-5-oxo-4,5-dihydropyrazin-2-yl)-2-methylphenyl)-4,5,6,7-tetrahydrobenzo[b]thiophene-2-carboxamide], CGI-560 4-(tert-butyl)-N-(3-(8-(phenylamino)imidazo[1,2-a]pyrazin-6-yl)phenyl)benzamide, CGI-1746 (4-(tert-butyl)-N-(2-methyl-3-(4-methyl-6-((4-(morpholine-4-carbonyl)phenyl)amino)-5-oxo-4,5-dihydropyrazin-2-yl)phenyl)benzamide), CNX-774 (4-(4-((4-((3-acrylamidophenyl)amino)-5-fluoropyrimidin-2-yl)amino)phenoxy)-N-methylpicolinamide), CTA056 (7-benzyl-1-(3-(piperidin-1-yl)propyl)-2-(4-(pyridin-4-yl)phenyl)-1H-imidazo[4,5-g]quinoxalin-6(5H)-one), GDC-0834 ((R)—N-(3-(6-((4-(1,4-dimethyl-3-oxopiperazin-2-yl)phenyl)amino)-4-methyl-5-oxo-4,5-dihydropyrazin-2-yl)-2-methylphenyl)-4,5,6,7-tetrahydrobenzo[b]thiophene-2-carboxamide), GDC-0837 ((R)—N-(3-(6-((4-(1,4-dimethyl-3-oxopiperazin-2-yl)phenyl)amino)-4-methyl-5-oxo-4,5-dihydropyrazin-2-yl)-2-methylphenyl)-4,5,6,7-tetrahydrobenzo[b]thiophene-2-carboxamide), HM-71224, ACP-196, ONO-4059 (Ono Pharmaceuticals), PRT062607 (4-((3-(2H-1,2,3-triazol-2-yl)phenyl)amino)-2-(((1R,2S)-2-aminocyclohexyl)amino)pyrimidine-5-carboxamide hydrochloride), QL-47 (1-(1-acryloylindolin-6-yl)-9-(1-methyl-1H-pyrazol-4-yl)benzo[h][1,6]naphthyridin-2(1H)-one), and RN486 (6-cyclopropyl-8-fluoro-2-(2-hydroxymethyl-3-{1-methyl-5-[5-(4-methyl-piperazin-1-yl)-pyridin-2-ylamino]-6-oxo-1,6-dihydro-pyridin-3-yl}-phenyl)-2H-isoquinolin-1-one), BGB-3111, and other molecules capable of inhibiting BTK activity, for example those BTK inhibitors disclosed in Akinleye et ah, Journal of Hematology & Oncology, 2013, 6:59, the entirety of which is incorporated herein by reference.

Syk inhibitors are well known, and include, for example, Cerdulatinib (4-(cyclopropylamino)-2-((4-(4-(ethylsulfonyl)piperazin-1-yl)phenyl)amino)pyrimidine-5-carboxamide), entospletinib (6-(1H-indazol-6-yl)-N-(4-morpholinophenyl)imidazo[1,2-a]pyrazin-8-amine), fostamatinib ([6-({5-Fluoro-2-[(3,4,5-trimethoxyphenyl)amino]-4-pyrimidinyl}amino)-2,2-dimethyl-3-oxo-2,3-dihydro-4H-pyrido[3,2-b][1,4]oxazin-4-yl]methyl dihydrogen phosphate), fostamatinib disodium salt (sodium (6-((5-fluoro-2-((3,4,5-trimethoxyphenyl)amino)pyrimidin-4-yl)amino)-2,2-dimethyl-3-oxo-2H-pyrido[3,2-b][1,4]oxazin-4(3H)-yl)methyl phosphate), BAY 61-3606 (2-(7-(3,4-Dimethoxyphenyl)-imidazo[1,2-c]pyrimidin-5-ylamino)-nicotinamide HCl), R09021 (6-[(1R,2S)-2-Amino-cyclohexylamino]-4-(5,6-dimethyl-pyridin-2-ylamino)-pyridazine-3-carboxylic acid amide), imatinib (Gleevec; 4-[(4-methylpiperazin-1-yl)methyl]-N-(4-methyl-3-{[4-(pyridin-3-yl)pyrimidin-2-yl]amino}phenyl)benzamide), staurosporine, GSK143 (2-(((3R,4R)-3-aminotetrahydro-2H-pyran-4-yl)amino)-4-(p-tolylamino)pyrimidine-5-carboxamide), PP2 (1-(tert-butyl)-3-(4-chlorophenyl)-1H-pyrazolo[3,4-d]pyrimidin-4-amine), PRT-060318 (2-(((1R,2S)-2-aminocyclohexyl)amino)-4-(m-tolylamino)pyrimidine-5-carboxamide), PRT-062607 (4-((3-(2H-1,2,3-triazol-2-yl)phenyl)amino)-2-(((1R,2S)-2-aminocyclohexyl)amino)pyrimidine-5-carboxamide hydrochloride), R112 (3,3′-((5-fluoropyrimidine-2,4-diyl)bis(azanediyl))diphenol), R348 (3-Ethyl-4-methylpyridine), R406 (6-((5-fluoro-2-((3,4,5-trimethoxyphenyl)amino)pyrimidin-4-yl)amino)-2,2-dimethyl-2H-pyrido[3,2-b][1,4]oxazin-3(4H)-one), YM193306(see Singh et al. Discovery and Development of Spleen Tyrosine Kinase (SYK) Inhibitors, J. Med. Chem. 2012, 55, 3614-3643), 7-azaindole, piceatannol, ER-27319 (see Singh et al. Discovery and Development of Spleen Tyrosine Kinase (SYK) Inhibitors, J. Med. Chem. 2012, 55, 3614-3643 incorporated in its entirety herein), Compound D (see Singh et al. Discovery and Development of Spleen Tyrosine Kinase (SYK) Inhibitors, J. Med. Chem. 2012, 55, 3614-3643 incorporated in its entirety herein), PRT060318 (see Singh et al. Discovery and Development of Spleen Tyrosine Kinase (SYK) Inhibitors, J. Med. Chem. 2012, 55, 3614-3643 incorporated in its entirety herein), luteolin (see Singh et al. Discovery and Development of Spleen Tyrosine Kinase (SYK) Inhibitors, J. Med. Chem. 2012, 55, 3614-3643 incorporated in its entirety herein), apigenin (see Singh et al. Discovery and Development of Spleen Tyrosine Kinase (SYK) Inhibitors, J. Med. Chem. 2012, 55, 3614-3643 incorporated in its entirety herein), quercetin (see Singh et al. Discovery and Development of Spleen Tyrosine Kinase (SYK) Inhibitors, J. Med. Chem. 2012, 55, 3614-3643 incorporated in its entirety herein), fisetin (see Singh et al. Discovery and Development of Spleen Tyrosine Kinase (SYK) Inhibitors, J. Med. Chem. 2012, 55, 3614-3643 incorporated in its entirety herein), myricetin (see Singh et al. Discovery and Development of Spleen Tyrosine Kinase (SYK) Inhibitors, J. Med. Chem. 2012, 55, 3614-3643 incorporated in its entirety herein), morin (see Singh et al. Discovery and Development of Spleen Tyrosine Kinase (SYK) Inhibitors, J. Med. Chem. 2012, 55, 3614-3643 incorporated in its entirety herein).

The chemotherapeutic agent that may be combined with the active compounds described herein for the treatment of cancer can also be a B-cell lymphoma 2 (Bcl-2) protein inhibitor. BCL-2 inhibitors are known in the art, and include, for example, ABT-199 (4-[4-[[2-(4-Chlorophenyl)-4,4-dimethylcyclohex-1-en-1-yl]methyl]piperazin-1-yl]-N-[[3-nitro-4-[[(tetrahydro-2H-pyran-4-yl)methyl]amino]phenyl]sulfonyl]-2-[(IH-pyrrolo[2,3-b]pyridin-5-yl)oxy]benzamide), ABT-737 (4-[4-[[2-(4-chlorophenyl)phenyl]methyl]piperazin-1-yl]-N-[4-[[(2R)-4-(dimethylamino)-1-phenylsulfanylbutan-2-yl]amino]-3-nitrophenyl]sulfonylbenzamide), ABT-263 ((R)-4-(4-((4′-chloro-4,4-dimethyl-3,4,5,6-tetrahydro-[I, I′-biphenyl]-2-yl)methyl)piperazin-1-yl)-N-((4-((4-morpholino-1-(phenylthio)butan-2-yl)amino)-3((trifluoromethyl)sulfonyl)phenyl)sulfonyl)benzamide), GX15-070 (obatoclax mesylate, (2Z)-2-[(5Z)-5-[(3,5-dimethyl-IH-pyrrol-2-yl)methylidene]-4-methoxypyrrol-2-ylidene]indole; methanesulfonic acid))), 2-methoxy-antimycin A3, YC137 (4-(4,9-dioxo-4,9-dihydronaphtho[2,3-d]thiazol-2-ylamino)-phenyl ester), pogosin, ethyl 2-amino-6-bromo-4-(1-cyano-2-ethoxy-2-oxoethyl)-4H-chromene-3-carboxylate, Nilotinib-d3, TW-37 (N-[4-[[2-(1,1-Dimethylethyl)phenyl]sulfonyl]phenyl]-2,3,4-trihydroxy-5-[[2-(1-methylethyl)phenyl]methyl]benzamide), Apogossypolone (ApoG2), or G3139 (Oblimersen).

Additional chemotherapeutic agents that may be combined with the active compounds described herein for the treatment of cancer for use in the methods contemplated herein include, but are not limited to, midazolam, MEK inhibitors, RAS inhibitors, ERK inhibitors, ALK inhibitors, HSP inhibitors (for example, HSP70 and HSP 90 inhibitors, or a combination thereof), RAF inhibitors, apoptotic compounds, topoisomerase inhibitors, AKT inhibitors, including but not limited to, MK-2206, GSK690693, Perifosine, (KRX-0401), GDC-0068, Triciribine, AZD5363, Honokiol, PF-04691502, and Miltefosine, or FLT-3 inhibitors, including but not limited to, P406, Dovitinib, Quizartinib (AC220), Amuvatinib (MP-470), Tandutinib (MLN518), ENMD-2076, and KW-2449, or combinations thereof. Examples of MEK inhibitors include but are not limited to trametinib/GSK1120212 (N-(3-{3-Cyclopropyl-5-[(2-fluoro-4-iodophenyl)amino]-6,8-dimethyl-2,4,7-trioxo-3,4,6,7-tetrahydropyrido[4,3-d]pyrimidin-1(2H-yl}phenyl)acetamide), selumetinib (6-(4-bromo-2-chloroanilino)-7-fluoro-N-(2-hydroxyethoxy)-3-methylbenzimidazole-5-carboxamide), pimasertib/AS703026/MSC1935369 ((S)-N-(2,3-dihydroxypropyl)-3-((2-fluoro-4-iodophenyl)amino)isonicotinamide), XL-518/GDC-0973 (1-({3,4-difluoro-2-[(2-fluoro-4-iodophenyl)amino]phenyl}carbonyl)-3-[(2S)-piperidin-2-yl]azetidin-3-ol), refametinib/BAY869766/RDEA119 (N-(3,4-difluoro-2-(2-fluoro-4-iodophenylamino)-6-methoxyphenyl)-1-(2,3-dihydroxypropyl)cyclopropane-1-sulfonamide), PD-0325901 (N-[(2R)-2,3-Dihydroxypropoxy]-3,4-difluoro-2-[(2-fluoro-4-iodophenyl)amino]-benzamide), TAK733 ((R)-3-(2,3-Dihydroxypropyl)-6-fluoro-5-(2-fluoro-4-iodophenylamino)-8-methylpyrido[2,3d]pyrimidine-4,7(3H,8H)-dione), MEK162/ARRY438162 (5-[(4-Bromo-2-fluorophenyl)amino]-4-fluoro-N-(2-hydroxyethoxy)-1-methyl-1H-benzimidazole-6 carboxamide), R05126766 (3-[[3-Fluoro-2-(methylsulfamoylamino)-4-pyridyl]methyl]-4-methyl-7-pyrimidin-2-yloxychromen-2-one), WX-554, R04987655/CH4987655 (3,4-difluoro-2-((2-fluoro-4-iodophenyl)amino)-N-(2-hydroxyethoxy)-5-((3-oxo-1,2-oxazinan-2 yl)methyl)benzamide), or AZD8330 (2-((2-fluoro-4-iodophenyl)amino)-N-(2-hydroxyethoxy)-1,5-dimethyl-6-oxo-1,6-dihydropyridine-3-carboxamide). Examples of RAS inhibitors include but are not limited to Reolysin and siG12D LODER. Examples of ALK inhibitors include but are not limited to Crizotinib, AP26113, and LDK378. HSP inhibitors include but are not limited to Geldanamycin or 17-N-Allylamino-17-demethoxygeldanamycin (17AAG), and Radicicol.

Known ERK inhibitors include SCH772984 (Merck/Schering-Plough), VTX-Ile (Vertex), DEL-22379, Ulixertinib (BVD-523, VRT752271), GDC-0994, FR 180204, XMD8-92, and ERK5-IN-1.

Raf inhibitors are well known, and include, for example, Vemurafinib (N-[3-[[5-(4-Chlorophenyl)-1H-pyrrolo[2,3-b]pyridin-3-yl]carbonyl]-2,4-difluorophenyl]-1-propanesulfonamide), sorafenib tosylate (4-[4-[[4-chloro-3-(trifluoromethyl)phenyl]carbamoylamino]phenoxy]-N-methylpyridine-2-carboxamide; 4-methylbenzenesulfonate), AZ628 (3-(2-cyanopropan-2-yl)-N-(4-methyl-3-(3-methyl-4-oxo-3,4-dihydroquinazolin-6-ylamino)phenyl)benzamide), NVP-BHG712 (4-methyl-3-(1-methyl-6-(pyridin-3-yl)-1H-pyrazolo[3,4-d]pyrimidin-4-ylamino)-N-(3-(trifluoromethyl)phenyl)benzamide), RAF-265 (1-methyl-5-[2-[5-(trifluoromethyl)-1H-imidazol-2-yl]pyridin-4-yl]oxy-N-[4-(trifluoromethyl)phenyl]benzimidazol-2-amine), 2-Bromoaldisine (2-Bromo-6,7-dihydro-1H,5H-pyrrolo[2,3-c]azepine-4,8-dione), Raf Kinase Inhibitor IV (2-chloro-5-(2-phenyl-5-(pyridin-4-yl)-1H-imidazol-4-yl)phenol), and Sorafenib N-Oxide (4-[4-[[[[4-Chloro-3(trifluoroMethyl)phenyl]amino]carbonyl]amino]phenoxy]-N-Methyl-2pyridinecarboxaMide 1-oxide).

Known topoisomerase I inhibitors useful in the present disclosure include (S)-10-[(dimethylamino)methyl]-4-ethyl-4,9-dihydroxy-1H-pyrano[3′,4′:6,7]indolizino[1,2-b]quinoline-3,14(4H,12H)-dione monohydrochloride (topotecan), (S)-4-ethyl-4-hydroxy-1H-pyrano[3′,4′:6,7]indolizino[1,2-b]quinoline-3,14-(4H,12H)-dione (camptothecin), (1S,9S)-1-Amino-9-ethyl-5-fluoro-1,2,3,9,12,15-hexahydro-9-hydroxy-4-methyl-10H,13H-benzo(de)pyrano(3′,4′:6,7)indolizino(1,2-b)quinoline-10,13-dione (exatecan), (7-(4-methylpiperazinomethylene)-10,11-ethylenedioxy-20(S)-camptothecin (lurtotecan), or (S)-4,11-diethyl-3,4,12,14-tetrahydro-4-hydroxy-3,14-dioxo1H-pyrano[3′,4′:6,7]-indolizino[1,2-b]quinolin-9-yl-[1,4′bipiperidine]-1′-carboxylate (irinotecan), (R)-5-ethyl-9,10-difluoro-5-hydroxy-4,5-dihydrooxepino[3′,4′:6,7]indolizino[1,2-b]quinoline-3,15(1H,13H)-dione (diflomotecan), (4S)-11-((E)-((1,1-Dimethylethoxy)imino)methyl)-4-ethyl-4-hydroxy-1,12-dihydro-14H-pyrano(3′,4′:6,7)indolizino(1,2-b)quinoline-3,14(4H)-dione (gimatecan), (S)-8-ethyl-8-hydroxy-15-((4-methylpiperazin-1-yl)methyl)-11,14-dihydro-2H-[1,4]dioxino[2,3-g]pyrano[3′,4′:6,7]indolizino[1,2-b]quinoline-9,12(3H,8H)-dione (lurtotecan), (4S)-4-Ethyl-4-hydroxy-11-[2-[(1-methylethyl)amino]ethyl]-1H-pyrano[3,4:6,7]indolizino[1,2-b]quinoline-3,14(4H,12H)-dione (belotecan), 6-((1,3-dihydroxypropan-2-yl)amino)-2,10-dihydroxy-12-((2R,3R,4S,5S,6R)-3,4,5-trihydroxy-6-(hydroxymethyl)tetrahydro-2H-pyran-2-yl)-12,13-dihydro-5H-indolo[2,3-a]pyrrolo[3,4-c]carbazole-5,7(6H)-dione (edotecarin), 8,9-dimethoxy-5-(2-N,N-dimethylaminoethyl)-2,3-methylenedioxy-5H-dibenzo(c,h)(1,6)naphthyridin-6-one (topovale), benzo[6,7]indolizino[1,2-b]quinolin-11(13H)-one (rosettacin), (S)-4-ethyl-4-hydroxy-11-(2-(trimethylsilyl)ethyl)-1H-pyrano[3′,4′:6,7]indolizino[1,2-b]quinoline-3,14(4H,12H)-dione (cositecan), tetrakis{(4S)-9-[([1,4′-bipiperidinyl]-1′-carbonyl)oxy]-4,11-diethyl-3,14-dioxo-3,4,12,14-tetrahydro-1H-pyrano[3′,4′:6,7]indolizino[1,2-b]quinolin-4-yl}N,N′,N″,N″′-{methanetetrayltetrakis[methylenepoly(oxyethylene)oxy(1-oxoethylene)]}tetraglycinate tetrahydrochloride (etirinotecan pegol), 10-hydroxy-camptothecin (HOCPT), 9-nitrocamptothecin (rubitecan), SN38 (7-ethyl-10-hydroxycamptothecin), and 10-hydroxy-9-nitrocamptothecin (CPT109), (R)-9-chloro-5-ethyl-5-hydroxy-10-methyl-12-((4-methylpiperidin-1-yl)methyl)-4,5-dihydrooxepino[3′,4′:6,7]indolizino[1,2-b]quinoline-3,15(1H,13H)-dione (elmotecan).

### C5 Inhibitor Combinations

Provided herein are methods for a treating a complement mediated disorder in a subject comprising administering to the subject an effective amount of a C5 inhibitor in combination or alternation with an effective amount of an active compound as described herein.

C5 inhibitors are known in the art. In some embodiments, the C5 inhibitor is a monoclonal antibody targeting C5. In some embodiments, the C5 inhibitor is eculizumab (SOLIRIS® Alexion Pharmaceuticals, Boston, MA, see, e.g., U.S. Pat. No. 9,352,035), or a biosimilar molecule thereof.

In some embodiments, the C5 inhibitor is ravulizumab (ULTOMIRIS® Alexion Pharmaceuticals, Boston, MA, see, e.g., U.S. Pat. Nos. 9,371,377; 9,079,949 and 9,633,574), or a biosimilar thereof.

In some embodiments, the C5 inhibitor may be, but is not limited to: a recombinant human minibody, for example Mubodina® (monoclonal antibody, Adienne Pharma and Biotech, Bergamo, Italy; see U.S. Pat. No. 7,999,081); coversin (nomacopan, Akari Therapeutics, London, England; see e.g., Penabad et al. Lupus, 2012, 23(12):1324-6); LFG316 (monoclonal antibody, Novartis, Basel, Switzerland, and Morphosys, Planegg, Germany; see U.S. Pat. Nos. 8,241,628 and 8,883,158); ARC-1905 (pegylated RNA aptamer, Ophthotech, Princeton, NJ and New York, NY; see Keefe et al., Nature Reviews Drug Discovery, 9, 537-550); RA101348 and zilucoplan (macrocyclic peptides, Ra Pharmaceuticals, Cambridge, MA); SOB1002 (affibody, Swedish Orphan Biovitrum, Stockholm, Sweden); cemdisiran (Si-RNA, Alnylam Pharmaceuticals, Cambridge, MA); ARC1005 (aptamers, Novo Nordisk, Bagsvaerd, Denmark); SOMAmers (aptamers, SomaLogic, Boulder, Co); SSL7 (bacterial protein toxin, see, e.g., Laursen et al. Proc. Natl. Acad. Sci. U.S.A., 107(8):3681-6); MEDI7814 (monoclonal antibody, Medlmmune, Gaithersburg, MD); aurin tricarboxylic acid; aurin tricarboxylic acid derivatives (Aurin Biotech, Vancouver, BC, see U.S. Patent Appl. Pub. 2013/003592); crovalimab (RG6107/SKY59; anti-C5 recycling antibody, Roche Pharmaceuticals, Basel, Switzerland); ALXN1210 and ALXN5500 (monoclonal antibodies, Alexion Pharmaceuticals, Boston, MA); TT30 (fusion protein, Alexion Pharmaceuticals, Boston, MA); REGN3918 (monoclonal antibody, Regeneron, Tarrytown, NY); ABP959 (eculizumab biosimilar, Amgen, Thousand Oaks, CA); BCD-148 (Biocad); and SB-12 (Samsung Bioepis Co., Ltd.); or combinations thereof.

In some embodiments, the C5 inhibitor is a recombinant human minibody, for example Mubodina®. Mubodina® is a fully human recombinant antibody C5 developed by Adienne Pharma and Biotech. Mubodina® is described in U.S. Pat. No. 7,999,081.

In some embodiments, the C5 inhibitor is coversin. Coversin is a recombinant protein derived from a protein discovered in the saliva of the Ornithodoros moubata tick currently developed as a recombinant protein by Akari Therapeutics (also known as nomacopan). Coversin is described in Penabad et al. Lupus 2012, 23(12):1324-6.

In some embodiments, the C5 inhibitor is Tesidolumab/LFG316. Tesidolumab is a monoclonal antibody developed by Novartis and Morphosys. Tesidolumab is described in U.S. Pat. Nos. 8,241,628 and 8,883,158.

In some embodiments, the C5 inhibitor is ARC-1905. ARC-1905 is a pegylated RNA aptamer developed by Ophthotech. ARC-1905 is described in Keefe et al. Nature Reviews Drug Discovery, 9:537-550.

In some embodiments, the C5 inhibitor is RA101348. RA101348 is a macrocyclic peptide developed by Ra Pharmaceuticals.

In some embodiments, the C5 inhibitor is RA101495. RA101495, also known as zilucoplan, is a macrocyclic peptide developed by Ra Pharmaceuticals.

In some embodiments, the C5 inhibitor is SOB1002. SOB1002 is an affibody developed by the Swedish Orphan Biovitrum.

In some embodiments, the C5 inhibitor is ARC1005. ARC1005 is an aptamer developed by Novo Nordisk.

In some embodiments, the C5 inhibitor is SOMAmers for C5. SOMAmers are aptamers developed by SomaLogic.

In some embodiments, the C5 inhibitor is SSL7. SSL7 is a bacterial protein toxin described in Laursen et al. Proc. Natl. Acad. Sci. U.S.A., 107(8):3681-6.

In some embodiments, the C5 inhibitor is MEDI7814. MEDI7814 is a monoclonal antibody developed by Medlmmune.

In some embodiments, the C5 inhibitor is aurin tricarboxylic acid. In another embodiment, the C5 inhibitor is an aurin tricarboxylic acid derivative. These aurin derivatives were developed by Aurin Biotech and are further described in U.S. Patent Appl. Pub. No. 2013/003592).

In some embodiments, the C5 inhibitor is RG6107/SKY59. RG6107/SKY59 is an anti-C5 recycling antibody developed by Roche Pharmaceuticals.

In some embodiments, the C5 inhibtior is ravulizumab (ULTOMIRIS®). In another embodiment, the C5 inhibitor is ALXN5500. Ravulizumab and ALXN5500 are monoclonal antibodies developed by Alexion Pharmaceuticals.

In some embodiments, the C5 inhibitor is TT30. TT30 is a fusion protein licensed by Alexion Pharmaceuticals.

In some embodiments, the C5 inhibitor is ABP959. ABP959 is an eculizamab biosimilar monoclonal antibody developed by Amgen.

In some embodiments, the C5 inhibtor is Anti-C5 siRNA cemdisiran. Anti-C5 siRNA was developed by Alnylam Pharmaceuticals.

In some embodiments, the C5 inhibitor is Erdigna®. Erdigna® is an antibody developed by Adienne Pharma.

In some embodiments, the C5 inhibitor is avacincaptad pegol/Zimura®. Avacincaptad pegol is in aptamer developed by Ophthotech.

In some embodiments, the C5 inhibitor is SOB1005. SOB1005 is a protein in developed by the Swedish Orphan Biovitrum.

In some embodiments, the C5 inhibitor is ISU305. ISU305 is a monoclonal antibody developed by ISU ABXIS.

In some embodiments, the C5 inhibitor is REGN3918. REGN3918 is a monoclonal antibody developed by Regeneron.

In some embodiments, the C5 inhibitor is BCD-148. BCD is an eculizumab biosimilar being developed by Biocad.

In some embodiments, the C5 inhibitor is SB-12. SB-12 is an eculizumab biosimilar being developed by Samsung Bioepis Co., Ltd.

### C3 Inhibitor Combinations

Provided herein are methods for treating a complement-mediated disorder in a subject comprising administering to the subject an effective amount of a C3 inhibitor in combination or alternation with an effective amount of an active compound described herein.

C3 inhibitors are known in the art. In some embodiments, a compound of the present disclosure is administered in combination or alternation with compstatin and/or a compstatin analog. Compstatin and compastin analogs are known and are found to be useful inhibitors of C3, see U.S. Pat. Nos. 9,056,076; 8,168,584; 9,421,240; 9,291,622; 8,580,735; 9,371,365; 9,169,307; 8,946,145; 7,989,589; 7,888,323; 6,319,897; and US Patent Appl. Pub. Nos. 2016/0060297; 2016/0015810; 2016/0215022; 2016/0215020; 2016/0194359; 2014/0371133; 2014/0323407; 2014/0050739; 2013/0324482; and 2015/0158915.

In some embodiments, the compstatin analog having the amino acid sequence ICVVQDWGHHCRT (SEQ. ID. NO. 1).

In another embodiment, the C3 inhibitor is a compstatin analog. In some embodiments, the compstatin analog is 4(1 MeW)/APL-1 of the sequence Ac-ICV(1-mW)QDWGAHRCT(SEQ. ID. NO. 2), wherein Ac is acetyl and 1-mW is 1-methyltryptophan.

In another embodiment, the compstatin analog is Cp40/AMY-101, which has an amino acid sequence ylCV(1 mW)QDW-Sar-AHRC-ml (SEQ. ID. NO. 3), wherein y is D-tyrosine, 1 mW is 1-methyltryptophan, Sar is sarcosine, and ml is N-methylisoleucine.

In yet another embodiment, the compstatin analog is PEG-Cp40, having the amino acid sequence PEG-ylCV(1 mW)QDW-Sar-AHRC-ml (SEQ. ID. NO. 4), wherein PEG is polyethyleneglycol (40 kDa), y is D-tyrosine, 1 mW is 1-methyltryptophan, Sar is sarcosine, and ml is N-methylisoleucine.

In yet another embodiment, the compstatin analog is 4(1MeVW)POT-4. 4(1MeVW)POT-4 was developed by Potentia.

In yet another embodiment, the compstatin analog is AMY-201. AMY-201 was developed by Amyndas Pharmaceuticals.

In some embodiments, a compound of the present disclosure can be combined with C3 inhibitors that include, but are not limited to: H17 (monoclonal antibody, EluSys Therapeutics, Pine Brook, NJ); mirococept (CR1-based protein); sCR1 (CR1-based protein, Celldex, Hampton, NJ); TT32 (CR-1 based protein, Alexion Pharmaceuticals, Boston, MA); HC-1496 (recombinant peptide); CB 2782 (enzyme, Catalyst Biosciences, South San Francisco, CA); APL-2 (pegylated synthetic cyclic peptide, Apellis Pharmaceuticals, Crestwood, KY); or combinations thereof.

In some embodiments, the C3 inhibitor is H17. H17 is a humanized monoclonal antibody in development by EluSys Therapeutics. H17 is described in Paixao-Cavalcante et al. J. Immunol. 2014, 192(10):4844-4851.

In some embodiments, the C3 inhibitor is mirococept. Mirococept is a CR1-based protein developed by Inflazyme Pharmaceuticals.

In some embodiments, the C3 inhibitor is sCR1. sCR1 is a soluble form of the CR1 protein developed by Celldex.

In some embodiments, the C3 inhibitor is TT32. TT32 is a CR-1 based protein licensed by Alexion Pharmaceuticals.

In some embodiments, the C3 inhibitor is HC-1496. HC-1496 is a recombinant peptide developed by InCode.

In some embodiments, the C3 inhibitor is CB 2782. CB 2782 is novel protease derived from human membrane type serine protease 1 (MTSP-1) that was developed by Catalyst Biosciences.

In some embodiments, the C3 inhibitor is APL-2. APL-2 is a pegylated version of APL-1 developed by Apellis Pharmaceuticals.

### Complement Factor B (CFB) Inhibitor Combinations

Provided herein are methods for treating complement mediated disorder comprising administering a CFB inhibitor in combination or alternation with an active compound of the present disclosure. CFB inhibitors are known in the art.

In some embodiments, a compound of the present disclosure can be combined with CFB inhibitors that include, but are not limited to: anti-FB SiRNA (Alnylam Pharmaceuticals, Cambridge, MA); TA106 (monoclonal antibody, Alexion Pharmaceuticals, Boston, MA); LNP023 (small molecule, Novartis, Basel, Switzerland); SOMAmers (aptamers, SomaLogic, Boulder, CO); bikaciomab (Novelmed Therapeutics, Cleveland, OH); complin (see, Kadam et al., J. Immunol. 2010, DOI:10.409/jimmunol.10000200); lonis-FB-L_{Rx} (ligand conjugated antisense drug, Ionis Pharmaceuticals, Carlsbad, CA); or a combination thereof.

In another embodiment, CFB inhibitors that can be combined with a compound of the present disclosure include those disclosed in PCT/US17/39587.

In another embodiment, CFB inhibitors that can be combined with a compound of the present disclosure as described herein include those disclosed in PCT/US17/014458.

In another embodiment, CFB inhibitors that can be combined with a compound of the present disclosure as described herein include those disclosed in U.S. Patent Appl. Pub. No. 2016/0024079; PCT Int. Appl. WO 2013/192345; PCT Int. Appl. WO 2013/164802; PCT Int. Appl. WO 2015/066241; PCT Int. Appl. WO 2015/009616 (assigned to Novartis AG).

In some embodiments, the CFB inhibitor is

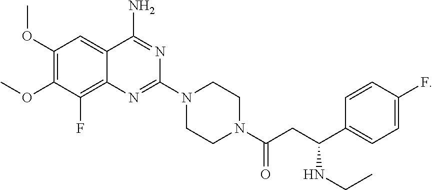 [SMILES: CCN[C@H](CC(=O)N1CCN(c2nc(N)c3cc(OC)c(OC)c(F)c3n2)CC1)c1ccc(F)cc1]

In another embodiment, the CFB inhibitor is

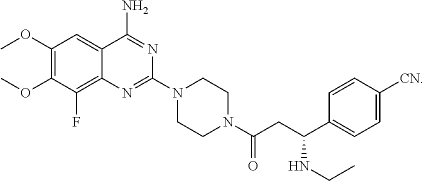 [SMILES: CCN[C@H](CC(=O)N1CCN(c2nc(N)c3cc(OC)c(OC)c(F)c3n2)CC1)c1ccc(C#N)cc1]

In another embodiment, the CFB inhibitor is

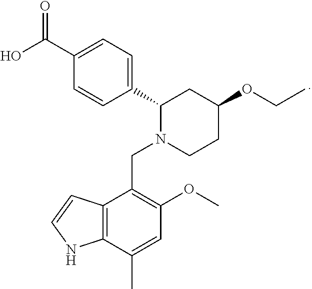 [SMILES: CCO[C@H]1CCN(Cc2c(OC)cc(C)c3[nH]ccc23)[C@H](c2ccc(C(=O)O)cc2)C1]

In some embodiments, the CFB inhibitor is anti-FB siRNA. Anti-FB siRNA was developed by Alnylam Pharmaceuticals.

In some embodiments, the CFB inhibitor is TA106. TA106 is a monoclonal antibody developed by Alexion Pharmaceuticals.

In some embodiments, the CFB inhibitor is LNPO23. LNPO23 is a small molecule inhibitor of CFB developed by Novartis.

In some embodiments, the CFB inhibitor is complin. Complin is a peptide inhibitor that is described in Kadam et al. J. Immunol. 2010 184(12):7116-24.

In some embodiments, the CFB inhibitor is IONIS-FB-LRx. IONIS-FB-LRx was developed by Ionis Pharmaceuticals.

### Complement Factor D (CFD) Inhibitor Combinations

Provided herein are methods for treating complement mediated disorder comprising administering a CFD inhibitor in combination or alternation with an active compound of the present disclosure.

In some embodiments, a fD inhibitor may be used as described by BioCryst Pharmaceuticals in U.S. Pat. No. 6,653,340 title “Compounds useful in the complement, coagulate and kallikrein pathways and methods for their preparation” which described fused bicyclic ring compounds that are potent inhibitors of Factor D.

In some embodiments, a fD inhibitor may be used as described by Novartis in PCT Patent Publication No. WO 2012/093101 titled “Indole compounds or analogues thereof useful for the treatment of age-related macular degeneration”. In another embodiment, a fD inhibitor may be used as described in Novartis PCT Patent Publication Nos. WO2013/164802, WO2013/192345, WO2014/002051, WO2014/002052, WO2014/002053, WO2014/002054, WO2014/002057, WO2014/002058, WO2014/002059, WO2014/005150, WO2014/009833, WO2014/143638, WO2015/009616, WO2015/009977, or WO2015/066241.

In some embodiments, a fD inhibitor may be used as described by Bristol-Myers Squibb in PCT Patent Publication No. WO2004/045518 titled “Open chain prolyl urea-related modulators of androgen receptor function”.

In some embodiments, a fD inhibitor may be used as described by Japan Tobacco Inc. in PCT Patent Publication No. WO1999/048492 title “Amide derivatives and nociceptin antagonists”.

In some embodiments, a fD inhibitor may be used as described by Ferring B. V. and Yamanouchi Pharmaceutical Co. LTD. in PCT Patent Publication No. WO 1993/020099 titled “CCK and/or gastrin receptor ligands”.

In some embodiments, the fD inhibitor is the monoclonal antibody FCFD4515S as developed by Genentech/Roche.

In some embodiments, the fD inhibitor is Nafamostat (FUT-175, Futhan) as developed by Torri Pharmaceuticals.

In some embodiments, the fD inhibitor is aptamers (SOMAmers) to Factor D as developed by SomaLogic.

In some embodiments, the fD inhibitor is the monoclonal antibody lampalizumab as developed by Roche.

In some embodiments, the fD inhibitor is aptamers to Factor D as developed by Vitrisa Therapeutics.

In some embodiments, the fD inhibitor is a fD inhibitor as developed by Ra Pharmaceuticals.

In some embodiments, the fD inhibitor comprises a drug disclosed in PCT/US17/014458.

In some embodiments, a fD inhibitor may be used as described by Alexion Pharmaceuticals in PCT Patent Publication No. WO1995/029697 title “Methods and compositions for the treatment of glomerulonephritis and other inflammatory diseases”.

In some embodiments, the fD inhibitor for use in combination with the compound of the disclosure is selected among those described by Achillion Pharmaceuticals in WO2015/130784; WO2015/130795; WO2015/130806; WO2015/130830; WO2015/130838; WO2015/130842; WO2015/130845; WO2015/130854; WO2016/044243; WO2017/035348; WO2017/035349; WO2017/035351; WO2017/035352; WO2017/035353; WO2017/035355; WO2017/035357; WO2017/035360; WO2017/035361; WO2017/035362; WO2017/035401; WO2017/035405; WO2017/035408; WO2017/035409; WO2017/035411; WO2017/035413; WO2017/035415; WO2017/035417; WO2017/035418; WO2018/160889; WO2018/160891; WO2018/160892; WO2019/028284; WO2019/028284; WO2019/227102; WO2020/041301; WO2020/051532; or WO2020/051538.

In some embodiments the fD inhibitor is a compound of Formula:

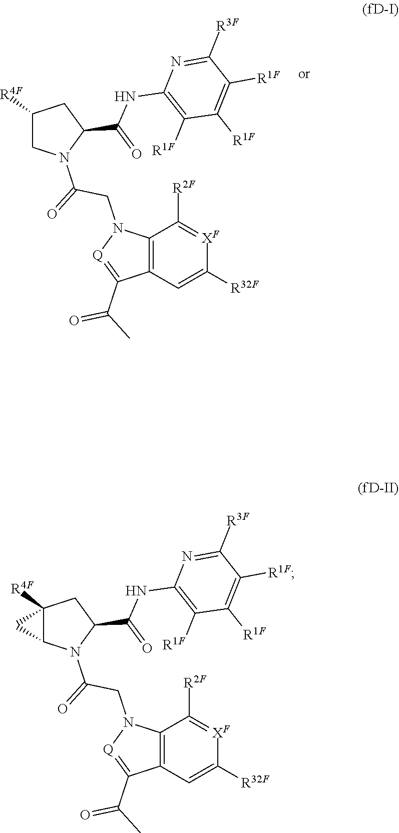

or a pharmaceutically acceptable salt thereof.
wherein:
Q is CH or N.
X^{F} is selected from N and CH;
each R^{1F} is independently selected from hydrogen, C₁-C₃ alkyl (e.g., methyl), and halogen (e.g., bromo, chloro, or fluoro);
R^{2F} is selected from hydrogen and C₁-C₃ alkyl (e.g., methyl);
R^{3F} is selected from C₁-C₃ alkyl (e.g., methyl), C₁-C₃ haloalkyl, and halogen (e.g., bromo, chloro, or fluoro);
R^{4F} is selected from hydrogen, C₁-C₃ alkyl (e.g., methyl), and halogen (e.g., bromo, chloro, or fluoro);
R^{5F} is selected from hydrogen, C₁-C₃ alkyl (e.g., methyl), halogen (e.g., bromo, chloro, or fluoro), -alkyl-OH, and cyano; and
R^{32F} is selected from

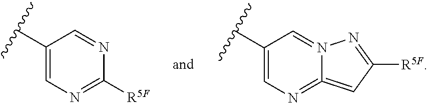

In some embodiments the fD inhibitor is selected from:

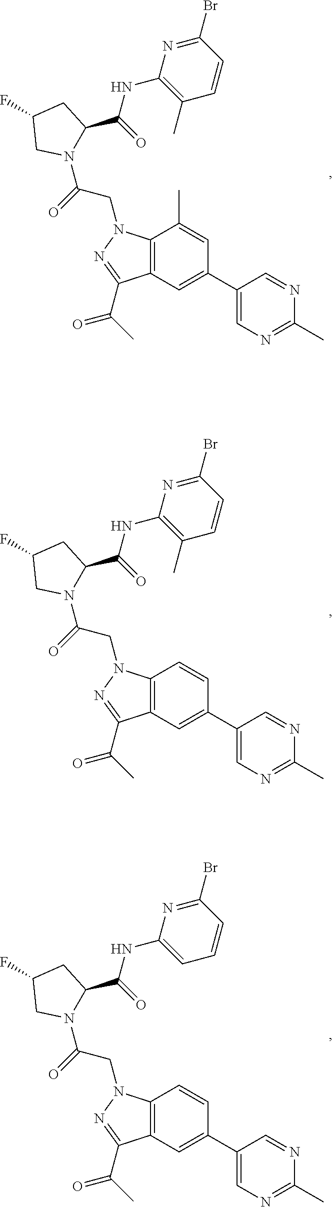 [SMILES: CC(=O)c1nn(CC(=O)N2C[C@H](F)C[C@H]2C(=O)Nc2nc(Br)ccc2C)c2c(C)cc(-c3cnc(C)nc3)cc12 ; CC(=O)c1nn(CC(=O)N2C[C@H](F)C[C@H]2C(=O)Nc2nc(Br)ccc2C)c2ccc(-c3cnc(C)nc3)cc12 ; CC(=O)c1nn(CC(=O)N2C[C@H](F)C[C@H]2C(=O)Nc2cccc(Br)n2)c2ccc(-c3cnc(C)nc3)cc12]

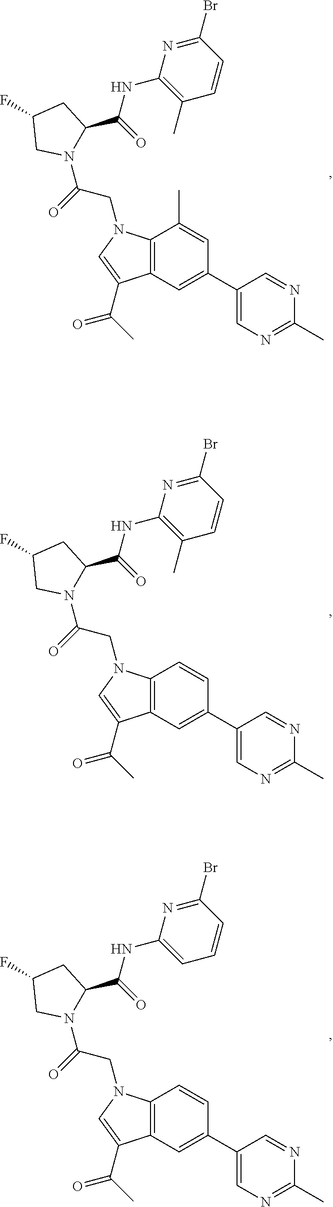 [SMILES: CC(=O)c1cn(CC(=O)N2C[C@H](F)C[C@H]2C(=O)Nc2nc(Br)ccc2C)c2c(C)cc(-c3cnc(C)nc3)cc12 ; CC(=O)c1cn(CC(=O)N2C[C@H](F)C[C@H]2C(=O)Nc2nc(Br)ccc2C)c2ccc(-c3cnc(C)nc3)cc12 ; CC(=O)c1cn(CC(=O)N2C[C@H](F)C[C@H]2C(=O)Nc2cccc(Br)n2)c2ccc(-c3cnc(C)nc3)cc12]

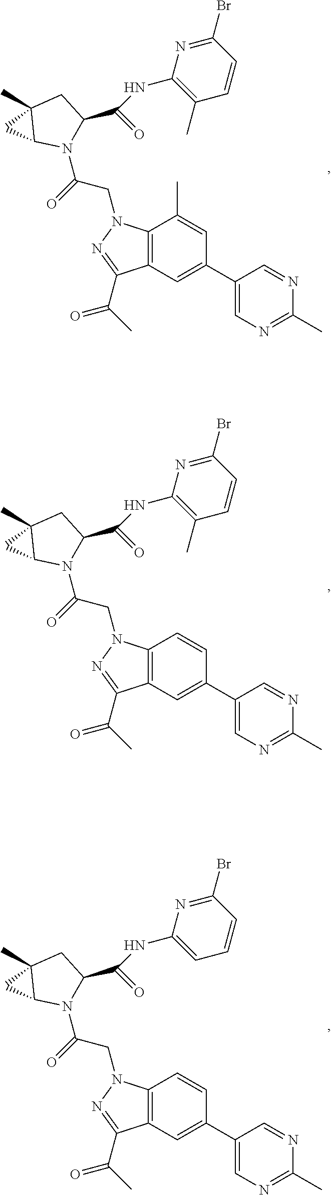

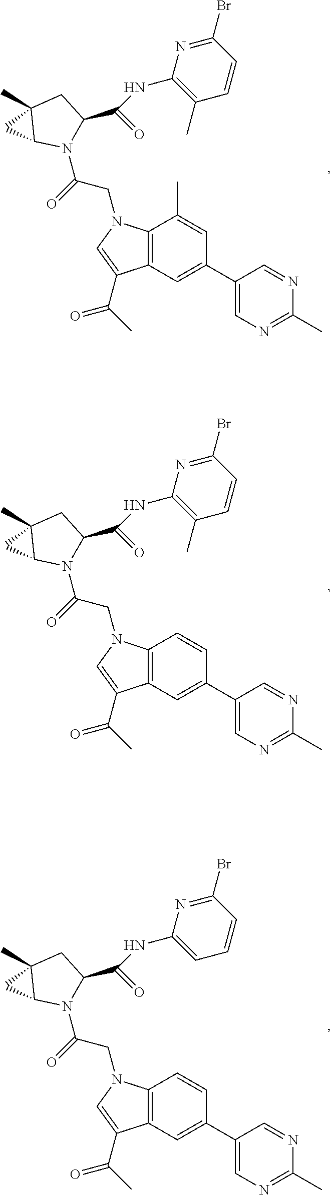

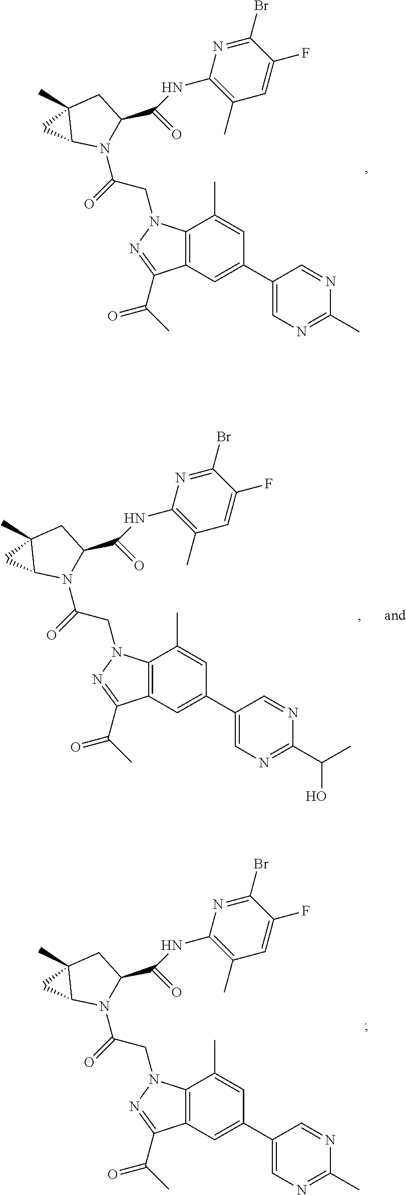

or a pharmaceutically acceptable salt thereof.

In some embodiments the fD inhibitor is selected from:

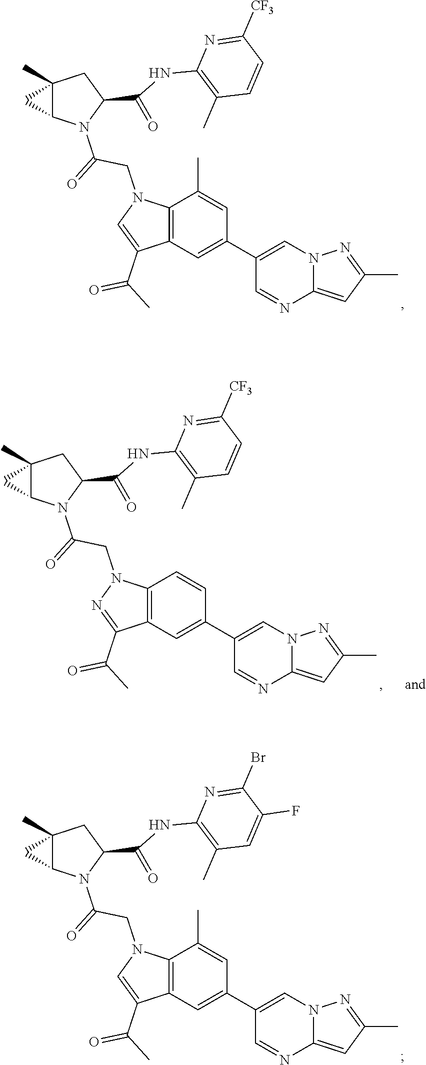 [SMILES: CC(=O)c1cn(CC(=O)N2[C@H](C(=O)Nc3nc(C(F)(F)F)ccc3C)C[C@@]3(C)C[C@@H]23)c2c(C)cc(-c3cnc4cc(C)nn4c3)cc12 ; CC(=O)c1nn(CC(=O)N2[C@H](C(=O)Nc3nc(C(F)(F)F)ccc3C)C[C@@]3(C)C[C@@H]23)c2ccc(-c3cnc4cc(C)nn4c3)cc12 ; CC(=O)c1cn(CC(=O)N2[C@H](C(=O)Nc3nc(Br)c(F)cc3C)C[C@@]3(C)C[C@@H]23)c2c(C)cc(-c3cnc4cc(C)nn4c3)cc12]

or a pharmaceutically acceptable salt thereof.

### Pan-inhibitors of Complement Components

Provided herein are methods for treating a complement mediated disorder comprising administering a pan-inhibitor of complement components in combination or alternation with a compound of the present disclosure. Pan-inhibitors of complement components are known in the art. In some embodiments, the inhibitor is FUT-175.

### Combinations for Prophylactic or Concommitant Anti-Bacterial Therapy

In one aspect of the present disclosure, a method is provided for treating a host in need thereof that comprises administering an effective amount of a prophylactic anti-bacterial vaccine prior to administration of an active compound or its salt or composition for any of the disorders described herein. In another aspect of the present disclosure, a method is provided for treating a host in need thereof that comprises administering an effective amount of a prophylactic anti-bacterial drug, such as a pharmaceutical drug, prior to administration of an active compound or its salt or composition for any of the disorders described herein. In one aspect of the present disclosure, a method is provided for treating a host in need thereof that comprises administering an effective amount of an anti-bacterial vaccine after administration of an active compound or its salt or composition for any of the disorders described herein. In another aspect of the present disclosure, a method is provided for treating a host in need thereof that comprises administering an effective amount of an anti-bacterial drug, such as a pharmaceutical drug, after administration of an active compound or its salt or composition for any of the disorders described herein. In one embodiment, the disorder is PNH, C3G, or aHUS. In one embodiment, the host has received an organ or other tissue or biological fluid transplant. In one embodiment, the host is also administered a C5 inhibitor, for example, eculizumab.

In one aspect of the present disclosure, an active compound or its salt or composition as described herein is administered to a host concomitantly to a subject following the prophylactic administration of a vaccine against a bacterial infection. In some embodiments, the complement-mediated disease or disorder is selected from the group consisting of autoimmune disease, cancer, hematological disease, infectious disease, inflammatory disease, ischemia-reperfusion injury, neurodegenerative disease, neurodegenerative disorder, ocular disease, renal disease, transplant rejection, vascular disease, and vasculitis disease. In one embodiment, the complement mediated disorder is PNH, C3G, or aHUS. In one embodiment, the subject has received an organ or other tissue or biological fluid transplant. In one embodiment, the subject is also administered eculizumab.

In one aspect of the present disclosure, an active compound or its salt or composition as described herein is administered to a subject concomitantly with the prophylactic administration of a vaccine against a bacterial infection. In some embodiments, the complement-mediated disease or disorder is selected from the group consisting of autoimmune disease, cancer, hematological disease, infectious disease, inflammatory disease, ischemia-reperfusion injury, neurodegenerative disease, neurodegenerative disorder, ocular disease, renal disease, transplant rejection, vascular disease, and vasculitis disease. In one embodiment, the complement mediated disorder is PNH, C3G, or aHUS. In one embodiment, the subject has received an organ or other tissue or biological fluid transplant. In one embodiment, the subject is also administered eculizumab.

In one aspect of the present disclosure, an active compound or its salt or composition as described herein is administered to a subject and, during the administration period of the compound or salt, a vaccine against a bacterial infection is administered to the subject. In some embodiments, the complement-mediated disease or disorder is selected from the group consisting of autoimmune disease, cancer, hematological disease, infectious disease, inflammatory disease, ischemia-reperfusion injury, neurodegenerative disease, neurodegenerative disorder, ocular disease, renal disease, transplant rejection, vascular disease, and vasculitis disease. In one embodiment, the complement mediated disorder is PNH, C3G, or aHUS. In one embodiment, the subject has received an organ or other tissue or biological fluid transplant. In one embodiment, the subject is also administered eculizumab.

In one aspect of the present disclosure, the subject is administered an active compound or its salt or composition as described herein in combination with an antibiotic compound for the duration of active compound administration. In some embodiments, the complement-mediated disease or disorder is selected from the group consisting of autoimmune disease, cancer, hematological disease, infectious disease, inflammatory disease, ischemia-reperfusion injury, neurodegenerative disease, neurodegenerative disorder, ocular disease, renal disease, transplant rejection, vascular disease, and vasculitis disease. In one embodiment, the complement mediated disorder is PNH, C3G, or aHUS. In one embodiment, the subject has received an organ or other tissue or biological fluid transplant. In one embodiment, the subject is also administered eculizumab.

In one aspect of the present disclosure, an active compound or its salt or composition as described herein is administered to a subject following the prophylactic administration of a vaccine against a bacterial infection, and in combination with an antibiotic compound for the duration of active compound administration. In some embodiments, the complement-mediated disease or disorder is selected from the group consisting of autoimmune disease, cancer, hematological disease, infectious disease, inflammatory disease, ischemia-reperfusion injury, neurodegenerative disease, neurodegenerative disorder, ocular disease, renal disease, transplant rejection, vascular disease, and vasculitis disease. In one embodiment, the complement mediated disorder is PNH or aHUS. In one embodiment, the subject has received an organ or other tissue or biological fluid transplant. In one embodiment, the subject is also administered eculizumab. In one embodiment, the subject, prior to receiving an active compound or its salt or composition as described herein, is vaccinated against a bacterial infection caused by the bacterium Neisseria meningitidis. In one embodiment, the subject is vaccinated against a bacterial infection caused by the bacterium Haemophilus influenzae. In one embodiment, the Haemophilus influenzae is Haemophilus influenzae serotype B (Hib).

In one embodiment, the subject is vaccinated against a bacterial infection caused by Streptococcus pneumoniae.

In one embodiment, the subject is vaccinated against a bacterial infection caused by the bacterium Nisseria meningitidis, Haemophilus influenzae, or Streptococcus pneumoniae, or a combination of one or more of Nisseria meningitidis, Haemophilus influenzae, or Streptococcus pneumoniae.

In one embodiment, the subject is vaccinated against a bacterial infection caused by the bacterium Nisseria meningitidis, Haemophilus influenzae, and Streptococcus pneumoniae.

In other embodiments, the subject is vaccinated against a bacterial infection caused by a bacterium selected from a Gram-negative bacterium.

In one embodiment, the subject is vaccinated against a bacterial infection caused by a bacterium selected from a Gram-positive bacterium.

In one embodiment, the subject is vaccinated against a bacterial infection caused by the bacterium Nisseria meningitidis, Haemophilus influenzae, or Streptococcus pneunemoniae, or a combination of one or more of Nisseria meningitidis, Haemophilus influenzae, or Streptococcus pneumoniae, and one or more of, but not limited to, Bacillus anthracis, Bordetella pertussis, Clostridium tetani, Corynebacterium diphtheria, Coxiella burnetii, Mycobacterium tuberculosis, Salmonella typhi, Vibrio cholerae, Anaplasma phagocytophilum, Ehrlichia ewingii, Ehrlichia chaffeensis, Ehrlichia canis, Neorickettsia sennetsu, Mycobacterium leprae, Borrelia burgdorferi, Borrelia mayonii, Borrelia afzelii, Borrelia garinii, Mycobacterium bovis, Staphylococcus aureus, Streptococcus pyogenes, Treponema pallidum, Francisella tularensis, and Yersinia pestis.

In one embodiment, the subject is vaccinated with one or more vaccines selected from, but not limited to, typhoid vaccine, live (Vivotif Berna Vaccine, PaxVax), typhoid Vi polysaccharide vaccine (Typhim Vi, Sanofi), pneumococcal 23-polyvalent vaccine, PCV13 (Pneumovax 23, Merck), pneumococcal 7-valent vaccine, PCV7 (Prevnar, Pfizer), pneumococcal 13-valent vaccine, PCV13 (Prevnar 13, Pfizer), haemophilus b conjugate (prp-t) vaccine (ActHIB, Sanofi; Hibrix, GSK), haemophilus b conjugate (hboc) vaccine (HibTITER, Neuron Biotech), haemophilus b conjugate (prp-omp) vaccine (PedvaxHlB, Merck), haemophilus b conjugate (prp-t) vaccine/meningococcal conjugate vaccine (MenHibrix, GSK), haemophilus b conjugate (prp-t) vaccine/meningococcal conjugate vaccine/Hepatitis B vaccine (Comvax, Merck), meningococcal polysaccharide vaccine (Menomune A/C/Y/W-135, Sanofi), meningococcal conjugate vaccine/diphtheria CRM197 conjugate (Menveo, GSK; Menactra, Sanofi), meningococcal group B vaccine (Bexsero, GSK; Trumenba, Pfizer), anthrax vaccine adsorbed (Biothrax, Emergent Biosolutions), tetanus toxoid (Te Anatoxal Berna, Hendricks Regional Health), Bacillus Calmette and Guerin, live, intravesical (TheraCys, Sanofi; Tice BCG, Organon), cholera vaccine, live, oral (Vachora, Sanofi; Dukoral, SBL Vaccines; ShanChol, Shantha Biotec; Micromedex, Truven Health), tetanus toxoids and diphtheria absorbed (Tdap; Decavac, Sanofi; Tenivac, Sanofi; td, Massachusetts Biological Labs), diphtheria and tetanus toxois and pertussis (DTap; Daptacel, Sanofi; Infanrix, GSK; Tripedia, Sanofi), diphtheria and tetanus toxois and pertussis/polio (Kinrix, GSK; Quadracel, Sanofi), diphtheria and tetanus toxois and pertussis tetanus/hepatitis B/polio (Pediarix, GSK), diphtheria and tetanus toxois and pertussis/polio, haemophilus influenza tybe b (Pentacel, Sanofi), and/or diphtheria, and pertussis (Tdap; Boostrix, GSK; Adacel, Sanofi), or a combination thereof.

As described above, a subject receiving a compound of the present disclosure to treat a disorder is prophylactically administered an antibiotic compound in addition to a compound described herein.

In one embodiment, the subject is administered an antibiotic compound for the duration of administration of the active compound to reduce the development of a bacterial infection.

Antibiotic compounds for concomitant administration with a compound described herein can be any antibiotic useful in preventing or reducing the effect of a bacterial infection. Antibiotics are well known in the art and include, but are not limited to, amikacin (Amikin), gentamicin (Garamycin), kanamycin (Kantrex), neomycin (Neo-Fradin), netilmicin (Netromycin), tobramycin (Nebcin), paromomycin (Humatin), streptomycin, spectinomycin (Trobicin), geldanamycin, herbimycin, rifaximin (Xifaxan), loracarbef (Lorabid), ertapenem (Invanz), doripenem (Doribax), imipenem/cilastatin (Primaxin), meropenem (Merrem), cefadroxil (Duricef), cefazolin (Ancef), cefalotin/cefalothin (Keflin), cephalexin (Keflex), cefaclor (Distaclor), cefamandole (Mandol), cefoxitin (Mefoxin), cefprozil (Cefzil), cefuroxime (Ceftin, Zinnat), cefixime (Cefspan), cefdinir (Omnicef, Cefdiel), cefditoren (Spectracef, Meiact), cefoperazone (Cefobid), cefotaxime (Claforan), cefpodoxime (Vantin) ceftazidime (Fortaz), ceftibuten (Cedax), ceftizoxime (Cefizox), ceftriaxone (Rocephin), cefepime (Maxipime), ceftaroline fosamil (Teflaro), ceftobiprole (Zeftera), teicoplanin (Targocid), vancomycin (Vancocin), telavancin (Vibativ), dalbavancin (Dalvance), oritavancin (Orbactiv), clindamycin (Cleocin), lincomycin (Lincocin), daptomycin (Cubicin), azithromycin (Zithromax, Sumamed, Xithrone), clarithromycin (Biaxin), dirithromycin (Dynabac), erythromycin (Erythocin, Erythroped), roxithromycin, troleandomycin (Tao), telithromycin (Ketek), spiramycin (Rovamycine), aztreonam (Azactam), furazolidone (Furoxone), nitrofurantoin (Macrodantin, Macrobid), linezolid (Zyvox), posizolid, radezolid, torezolid, amoxicillin (Novamox, Amoxil), ampicillin (Principen), azlocillin, carbenicillin (Geocillin), cloxacillin (Tegopen), dicloxacillin (Dynapen), flucloxacillin (Floxapen), mezlocillin (Mezlin), methicillin (Staphcillin), nafcillin (Unipen), oxacillin (Prostaphlin), penicillin G (Pentids), penicillin V (Veetids (Pen-Vee-K), piperacillin (Pipracil), penicillin G (Pfizerpen), temocillin (Negaban), ticarcillin (Ticar), amoxicillin/clavulanate (Augmentin), ampicillin/sulbactam (Unasyn), piperacillin/tazobactam (Zosyn), ticarcillin/clavulanate (Timentin), bacitracin, colistin (Coly-Mycin-S), polymyxin B, ciprofloxacin (Cipro, Ciproxin, Ciprobay), enoxacin (Penetrex), gatifloxacin (Tequin), gemifloxacin (Factive), levofloxacin (Levaquin), lomefloxacin (Maxaquin), moxifloxacin (Avelox), nalidixic acid (NegGram), norfloxacin (Noroxin), ofloxacin (Floxin, Ocuflox), trovafloxacin (Trovan), grepafloxacin (Raxar), sparfloxacin (Zagam), temafloxacin (Omniflox), mafenide (Sulfamylon), sulfacetamide (Sulamyd, Bleph-10), sulfadiazine (Micro-Sulfon), silver sulfadiazine (Silvadene), sulfadimethoxine (Di-Methox, Albon), sulfamethizole (Thiosulfil Forte), sulfamethoxazole (Gantanol), sulfanilamide, sulfasalazine (Azulfidine), sulfisoxazole (Gantrisin), trimethoprim-sulfamethoxazole (Co-trimoxazole) (TMP-SMX) (Bactrim, Septra), sulfonamidochrysoidine (Prontosil), demeclocycline (Declomycin), doxycycline (Vibramycin), minocycline (Minocin), oxytetracycline (Terramycin), tetracycline (Sumycin, Achromycin V, Steclin), clofazimine (Lamprene), dapsone (Avlosulfon), capreomycin (Capastat), cycloserine (Seromycin), ethambutol (Myambutol), ethionamide (Trecator), isoniazid (I.N.H.), pyrazinamide (Aldinamide), rifampicin (Rifadin, Rimactane), rifabutin (Mycobutin), rifapentine (Priftin), streptomycin, arsphenamine (Salvarsan), chloramphenicol (Chloromycetin), fosfomycin (Monurol, Monuril), fusidic acid (Fucidin), metronidazole (Flagyl), mupirocin (Bactroban), platensimycin, quinupristin/dalfopristin (Synercid), thiamphenicol, tigecycline (Tigacyl), tinidazole (Tindamax Fasigyn), trimethoprim (Proloprim, Trimpex), and/or teixobactin, or a combination thereof.

In one embodiment, the subject is administered a prophylactic antibiotic selected from cephalosporin, for example, ceftriaxone or cefotaxime, ampicillin-sulbactam, Penicillin G, ampicillin, chloramphenicol, fluoroquinolone, aztreonam, levofloxacin, moxifloxacin, gemifloxacin, vancomycin, clindamycin, cefazolin, azithromycin, meropenem, ceftaroline, tigecycline, clarithromycin, moxifloxacin, trimethoprim/sulfamethoxazole, cefuroxime, axetil, ciprofloxacin, rifampin, minocycline, spiramycin, and cefixime, or a combination of two or more thereof.

### Process of Preparation of Compounds of the Present Disclosure

### ABBREVIATIONS

ACN Acetonitrile
Ac Acetyl
Ac₂O Acetic anhydride
AcOEt, EtOAc ethyl acetate
AcOH Acetic acid
AcONa Sodium acetate
AICl₃ Aluminum chloride
BH₃ borane
Boc₂O di-tert-butyl dicarbonate
Boc₂NH Di-tert-butyl-iminodicarboxylate
BnBr Benzyl bromide
BnOH Benzyl alcohol
Bu Butyl
Bu₄NHSO₄ Tetrabutylammonium bisulfate
CAN Ceric ammonium nitrate
CBr₄ Carbon tetrabromide
CBz Carboxybenzyl
CDI Carbonyldiimidazole
CH₃OH,
Methanol
MeOH
CH₃PPh₃Br Methyltriphenylphosphonium bromide
CCl₃Br Bromotrichloromethane
(COCl)₂ oxalylchloride
ClCO₂Et ethyl chloroformate
(CN)Br cyanogen bromide
CsF Cesium fluoride
CuI Cuprous iodide
DAST Diethylaminosulfur trifluoride
DBU 1,8-Diazabicyclo[5.4.0]undec-7-ene,
DCM, CH₂Cl₂
Dichloromethane
DIBAL-H Diisobutylaluminium hydride
DIAD Diisopropyl azodicarboxylate
DIEA, DIPEA
N,N-diisopropylethylamine
DMA N,N-dimethylacetamide
DMAP 4-Dimethylaminopyridine
DMBNH2 3,5-Dimethoxybenzylamine
DMF N,N-dimethylformamide
DMS Dimethyl sulfide
DMSO Dimethylsulfoxide
DPPA Diphenyl phosphoryl azide
EDCI 1-Ethyl-3-(3-dimethylaminopropyl)carbodiimide
EEDQ N-Ethoxycarbonyl-2-ethoxy-1,2-dihydroquinoline
Et Ethyl
Et₃N, TEA Triethylamine
EtOAc Ethylacetate
EtOH Ethanol
FA Formic acid
HATU 1-[Bis(dimethylamino)methylene]-1H-1,2,3-triazolo[4,5-b]pyridinium3-oxide hexafluorophosphate
H2SO₄ Sulfuric acid
HBF₄ Fluoroboric acid
HBr-AcOH Acetic acid hydrogen bromide
HCl Hydrochloric acid
HOBT Hydroxybenzotriazole
iBu, i-Bu,
isoBu Isobutyl
iPr, i-Pr, isoPr Isopropyl
ⁱPr₂NEt N,N-diisopropylethylamine
KH₂PO₄ Potassium dihydrogen phosphate
K₂CO₃ Potassium carbonate
KI Potassium iodide
KMnO₄ Potassium permanganate
Li₂CO₃ Lithium carbonate
LiOH Lithium hydroxide
LiHMDS Lithium bis(trimethylsilyl)amide
Me Methyl
MeCN Acetonitrile
Mel Methyl iodide
Ms Mesyl
MsCl Mesylchloride
MTBE Methyltbutylether
n-BuLi n-Butyllithium
NaBH₄ sodium borohydride
NaBH₃CN Sodium cyanoborohydride
Na₂SO₄ Sodium sulfate
NaCl Sodium chloride
NaClO Sodium hypochlorite
NaH Sodium hydride
NaHCO₃ Sodium bicarbonate
NaI Sodium iodide
NaOH Sodium hydroxide
NBS N-bromo succinimide
NCS N-chloro succinimide
NEt₃ Trimethylamine
NH₂SO₃H Sulfamic acid
NH₂OH Hydroxylamine
NH₄OH Ammonium hydroxide
NH₄OAc Ammonium acetate
Ni nickel
NMP N-Methyl-2-pyrrolidone
PCC Pyridinium chlorochromate
Pd (OAc)₂ Palladium acetate
Pd(dppf)Cl₂ [1,1′-Bis(diphenylphosphino) ferrocene]dichloropalladium(II)
Pd(PPh₃)₂Cl₂ Bis(triphenylphosphine)palladium(II) dichloride
Pd(PPh₃)₄ Tetrakis(triphenylphosphine)palladium(0)
Pd/C Palladium on carbon
Pd₂(dba)₃ Tris(dibenzylideneacetone)dipalladium(0)
PMB 4-Methoxybenzyl ether
PPh₃ Triphenylphosphine
Pr Propyl
PtO₂ Platinum oxide
PTSA p-Toluenesulfonic acid
Py, py Pyridine
PyBOP benzotriazol-1-yl-oxytripyrrolidinophosphonium hexafluorophosphate
RT Room temperature
T₃P Propane phosphonic acid anhydride
TBAB Tetra-n-butylammonium bromide
TBAF Tetra-n-butylammonium fluoride
TBAT Tetrabutylammonium difluorotriphenylsilicate
tBu, t-Bu tertbutyl
tBuOK Potassium tert-butoxide
TEA Trimethylamine
TES Tris(hydroxymethyl)methyl]-2-aminoethanesulfonic acid
Tf₂O Trifluoromethanesulfonic anhydride
TFA Trifluoroacetic acid
TFAA Trifluoroacetic anhydride
THF Tetrahydrofuran
TiCl₄ Titanium tetrachloride
TMS Trimethylsilane
TMSBr Bromotrimethylsilane
TMSCHN₂ Trimethylsilyldiazomethane
tol. toluene
t_{R} Retention time
Troc 2,2,2-Trichlorethoxycarbonyl chloride
Zn (CN)₂ Zinc cyanide

### General Methods

All nonaqueous reactions were performed under an atmosphere of dry argon or nitrogen gas using anhydrous solvents. The progress of reactions and the purity of target compounds were determined using one of the two liquid chromatography (LC) methods A or B disclosed herein. The structure of starting materials, intermediates, and final products was confirmed by standard analytical techniques, including NMR spectroscopy and mass spectrometry.

### LC Method A

Instrument: Waters Acquity Ultra Performance LC
Column: ACQUITY UPLC BEH C18 2.1×50 mm, 1.7 μm
Column Temperature: 40° C.
Mobile Phase: Solvent A: H₂O+0.05% FA; Solvent B: CH₃CN+0.05% FA
Flow Rate: 0.8 mL/min
Gradient: 0.24 min @15% B, 3.5 min gradient (15-85% B), then 0.5 min @85% B.
Detection: UV (210-410 nm) and MS (SQ in ES+ mode)

### Example 1. Non-Limiting Synthetic Examples of Compounds of the Present Disclosure

The below schemes are non-limiting examples of methods to make compounds of the present disclosure. The skilled artisan will recognize that there are various modifications that can be performed to make analogs or prepare compounds in other ways.

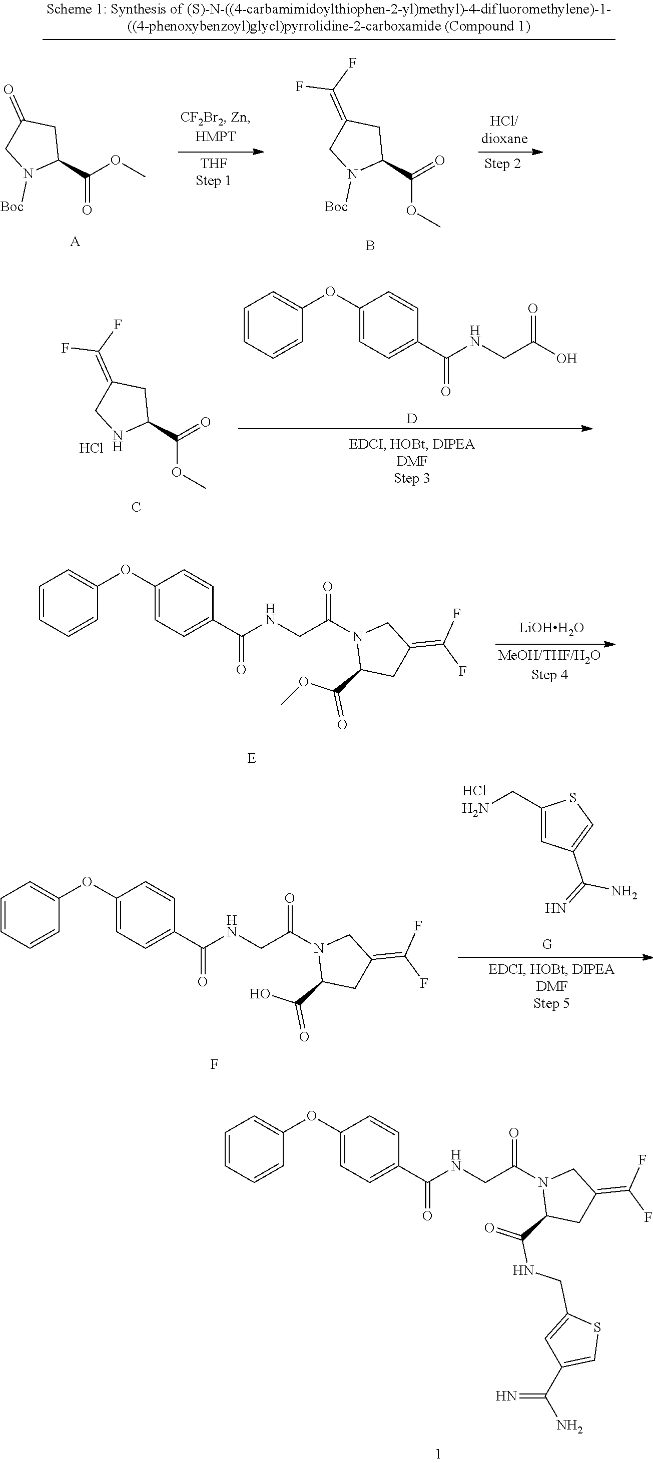

Step 1: 1-(tert-butyl) 2-methyl (S)-4-(difluoromethylene)pyrrolidine-1,2-dicarboxylate (B). To a solution of compound A (1 g, 4.1 mmol) in THE (40 mL) was added CF₂Br₂ (1.5 mL, 16.4 mmol) and HMPT (2.98 mL, 16.4 mmol) at 0° C. under N₂ atmosphere. Then activated zinc dust (1.07 g, 16.4 mmol) and another portion of HMPT (0.2 mL) were added and the reaction was stirred at 70° C. for 4 hours. The reaction was cooled to 20° C. and filtered through celatom. The filtrate was diluted with 20 mL H₂O and extracted with MTBE. The organic layer was dried over anhydrous Na₂SO₄, filtered and concentrated to dryness in vacuo. The residue was purified by column chromatography on silica gel (PE:EtOAc=100:1 to 30:1) to give compound B (500 mg, yield 44%) as a colorless oil. LC/MS (ESI) (m/z): 178 (M−100+H)⁺.

Step 2: methyl (S)-4-(difluoromethylene)pyrrolidine-2-carboxylate hydrochloride (C). Compound B (300 mg, 1.08 mmol) and HCl/1,4-dioxane (3 mL, 4M) were stirred at 10° C. for 2 hours. The reaction was concentrated in vacuo to give compound C (230 mg, yield 100%) as a yellow oil, which was used directly in the next step without further purification. LC/MS (ESI) (m/z): 178 (M+H)⁺.

Step 3: methyl (S)-4-(difluoromethylene)-1-((4-phenoxybenzoyl)glycyl)pyrrolidine-2-carboxylate (E). To a mixture of compound C (230 mg, 1.07 mmol) and compound D (226 mg, 0.83 mmol) in DMF (2 mL) was added DIPEA (0.55 mL, 3.32 mmol), followed by addition of HOBt (224 mg, 1.66 mmol) and EDCI (318 mg, 1.66 mmol) at 0° C. under N2 atmosphere. The mixture was stirred at 25° C. for 16 hours. The mixture was diluted with EtOAc and washed with saturated aq. NH₄Cl solution and brine. The organic layer was dried over anhydrous Na₂SO₄, filtered and concentrated to dryness under reduced pressure. The residue was purified by column chromatography on silica gel (DCM:MeOH=100:1 to 20:1) to give compound E (346 mg, yield 96.9%) as a white solid. LC/MS (ESI) m/z: 431 (M+H)⁺.

Step 4: (S)-4-(difluoromethylene)-1-((4-phenoxybenzoyl)glycyl)pyrrolidine-2-carboxylic acid (F). To a solution of compound E (200 mg, 0.465 mmol) in THE (2 mL), MeOH (0.5 mL) and H₂O (0.5 mL) was added LiOHH₂O (97.7 mg, 2.3 mmol) at 0° C. and the mixture was stirred at 25° C. for 1.5 hours. The mixture was diluted with water and extracted with EtOAc twice. The aqueous layer was acidified with 2M aq. HCl solution to pH=3 and washed with EtOAc twice. The organic layer was washed with brine, dried over anhydrous Na₂SO₄, filtered and concentrated to dryness under reduced pressure to give compound F (148 mg, yield 73.1%) as a white solid, which was used directly in the next step without further purification. LC/MS (ESI) m/z: 417 (M+H)⁺.

Step 5: (S)-N-((4-carbamimidoylthiophen-2-yl)methyl)-4-(difluoromethylene)-1-((4-phenoxybenzoyl)glycyl)pyrrolidine-2-carboxamide (Compound 1). To a mixture of compound F (80 mg, 0.192 mmol) and compound G (175 mg, 0.77 mmol) in DMF (2 mL) was added DIPEA (0.32 mL, 1.92 mmol) at 0° C. under N2 atmosphere, followed by addition of HOBt (78.3 mg, 0.58 mmol) and EDCI (110 mg, 0.58 mmol). The mixture was stirred at 25° C. for 16 hours. The mixture was diluted with EtOAc and washed with brine. The organic layer was dried over anhydrous Na₂SO₄, filtered and concentrated to dryness under reduced pressure. The residue was purified by prep-HPLC to give Compound 1 (15 mg, yield 14.1%) as a white solid. ¹H NMR (400 MHz, DMSO-d₆) δ 9.08-8.67 (m, 2H), 8.45 (s, 1H), 8.33 (dd, J=9.5, 1.4 Hz, 1H), 7.90 (dd, J=8.9, 2.2 Hz, 2H), 7.52-7.38 (m, 3H), 7.22 (t, J=7.4 Hz, 1H), 7.09 (d, J=7.8 Hz, 2H), 7.07-7.01 (m, 2H), 4.86-4.60 (m, 1H), 4.51-4.32 (m, 3H), 4.26-4.14 (m, 1H), 4.07-4.02 (m, 1H), 3.71-3.50 (m, 1H), 3.11-2.86 (m, 1H), 2.71-2.53 (m, 1H); LC/MS (ESI) (m/z): 554 (M+H)⁺.

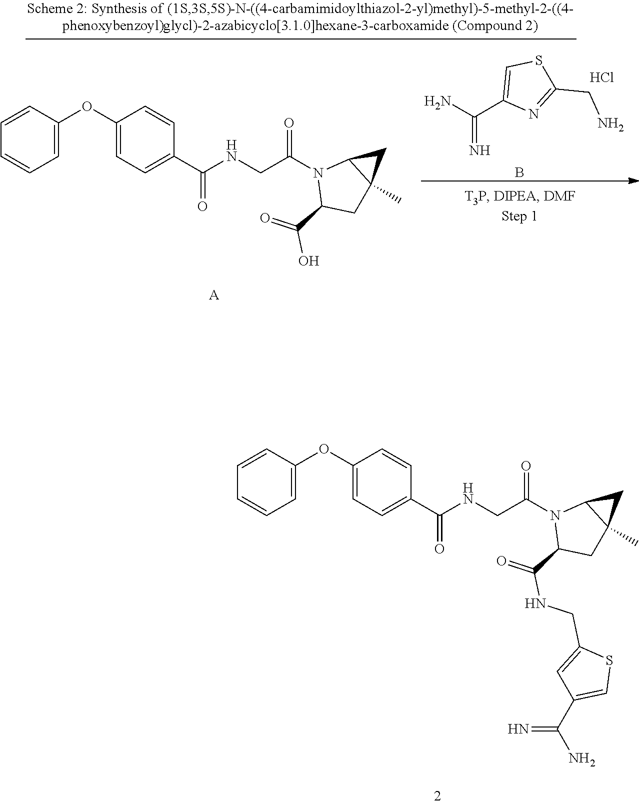

Step 1: (1S,3S,5S)-N-((4-carbamimidoylthiazol-2-yl)methyl)-5-methyl-2-((4-phenoxy-benzoyl)glycyl)-2-azabicyclo[3.1.0]hexane-3-carboxamide (Compound 2). To a mixture of compound A (63 mg, 0.16 mmol) and compound B (37 mg, 0.24 mmol) in DMF (3 mL) was added DIPEA (103 mg, 0.8 mmol) and T₃P (203 mg, 0.32 mmol, 50% wt in EtOAc) at 0° C. under N2 atmosphere. The resulting mixture was stirred at 25° C. overnight. The mixture was diluted with EtOAc and washed with water and brine. The organic layer was dried over anhydrous Na₂SO₄, filtered and concentrated to dryness under reduced pressure. The residue was purified by prep-HPLC to give Compound 2 (15.0 mg, yield 17.6%) as white solid. ¹H NMR (400 MHz, CDCl₃) δ 10.63 (s, 1H), 8.71 (d, J=61.2 Hz, 2H), 8.45-8.19 (m, 3H), 7.76 (d, J=8.4 Hz, 2H), 7.38-7.32 (m, 2H), 7.15 (t, J=7.4 Hz, 1H), 7.02 (d, J=8.0 Hz, 2H), 6.90 (d, J=8.4 Hz, 2H), 4.90-4.78 (m, 1H), 4.68-4.41 (m, 2H), 4.41-4.23 (m, 2H), 3.20 (dd, J=4.8, 4.8 Hz, 1H), 2.41-2.28 (m, 2H), 1.29 (s, 3H), 1.28-1.25 (m, 1H), 0.82 (t, J=5.6 Hz, 12H); LC/MS (ESI) (m/z): 533 (M+H)⁺.

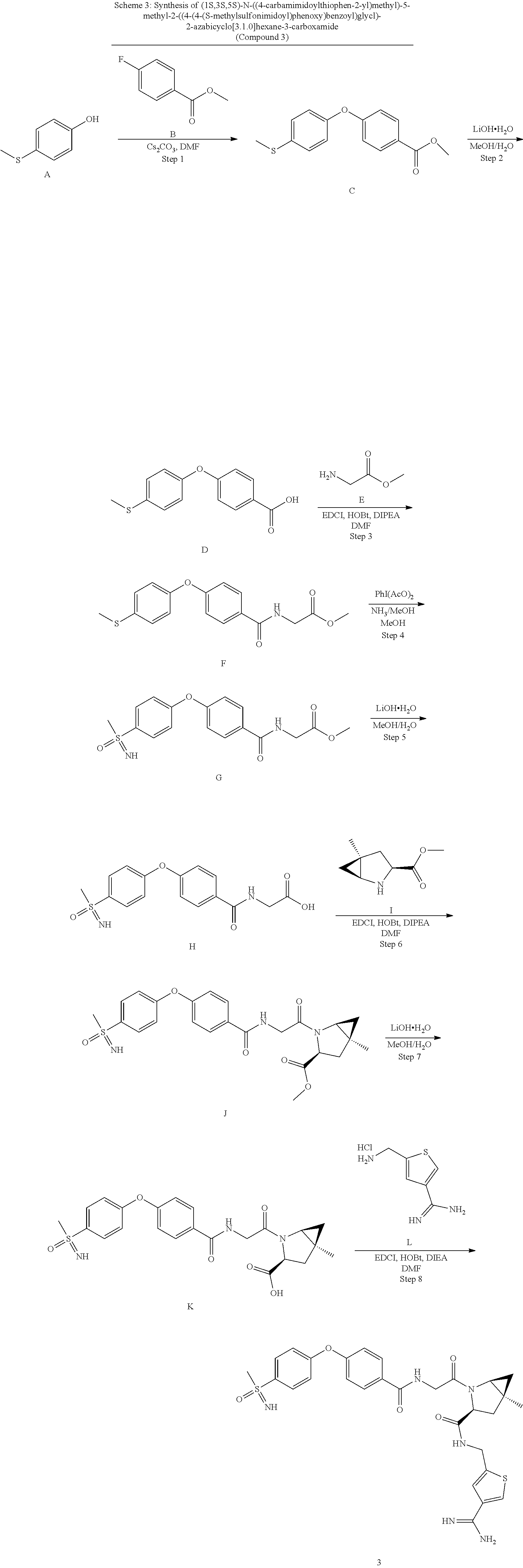

Step 1: methyl 4-(4-(methylthio)phenoxy)benzoate (C). To a solution of compound A (1.0 g, 7.13 mmol) in DMF (20 mL) was added compound B (1.32 g, 8.56 mmol) and Cs₂CO₃ (11.6 g, 35.65 mmol) at 25° C. The mixture was stirred at 120° C. for 16 hours. The mixture was cooled to 25° C. and diluted with EtOAc. The organic layer was washed with saturated aq.NH₄Cl solution and brine, dried over anhydrous Na₂SO₄, filtered and concentrated to dryness under reduced pressure. The residue was purified by column chromatography on silica gel (PE:EtOAc=9:1) to give compound 3 (1.36 g, yield 69.7%) as a white solid. LC/MS (ESI) (m/z): 275 (M+H)⁺.

Step 2: T4-(4-(methylthio)phenoxy)benzoic acid (D). To a solution of compound C (1.36 g, 5.0 mmol) in MeOH (10 mL) and water (2 mL) was added LiOH·H₂O (625 mg, 15 mmol) at 0° C. and the mixture was stirred at room temperature for 2 hours. The mixture was concentrated to 1/5 volume, diluted with water and washed with MTBE twice. The aqueous layer was acidified with 1 N aq. HCl to pH˜3 and extracted with DCM twice. The combined organic layers were washed with brine, dried over anhydrous Na₂SO₄, and concentrated to dryness under reduced pressure to give compound D (1.22 g, yield 94.6%) as a white solid, which was used directly in next step without further purification. LC/MS (ESI) (m/z): 261 (M+H)⁺.

Step 3: methyl (4-(4-(methylthio)phenoxy)benzoyl)glycinate (F). To a mixture of compound D (475 mg, 1.83 mmol) and compound E (344 mg, 2.75 mmol) in DMF (5 mL) was added DIPEA (1.5 mL, 9.15 mmol), EDCI (700 mg, 3.66 mmol) and HOBT (370 mg, 2.75 mmol) at 0° C. under N2 atmosphere. The reaction was stirred at room temperature for 16 hours and diluted with EtOAc. The mixture was washed with saturated aq.NH₄Cl solution and brine. The organic layer was dried over anhydrous Na₂SO₄, filtered and concentrated to dryness under reduced pressure. The residue was purified by column chromatography on silica gel (PE:EtOAc=3:2) to give compound F (560 mg, yield 92.6%) as a white solid. LC/MS (ESI) m/z: 332 (M+H)⁺.

Step 4: methyl (4-(4-(S-methylsulfonimidoyl)phenoxy)benzoyl)glycinate (G). To a solution of compound F (160 mg, 0.48 mmol) in MeOH (5 mL) was added NH₃/MeOH (0.1 mL, 0.72 mmol) and Phl(OAc)₂ (358 mg, 1.10 mmol) at 0° C. under N2 atmosphere and the mixture was stirred at room temperature for 1 hour. The mixture was diluted with EtOAc and washed with brine. The organic layer was dried over anhydrous Na₂SO₄, filtered and concentrated to dryness under reduced pressure. The residue was purified by column chromatography on silica gel (DCM:MeOH=20:1) to give compound G (170 mg, yield 97.1%) as a white solid. LC/MS (ESI) m/z: 363 (M+H)⁺.

Step 5: (4-(4-(S-methylsulfonimidoyl)phenoxy)benzoyl)glycine (H). To a solution of compound G (170 mg, 0.47 mmol) in MeOH (5 mL) and water (1 mL) was added a solution of LiOHH₂O (61 mg, 1.41 mmol) at 0° C. and the mixture was stirred at room temperature for 2 hours. The mixture was acidified with 1N aq. HCl to pH˜3 and concentrated to dryness under reduced pressure to give compound H (117 mg, yield 69.6%) as a light-yellow solid, which was used directly in next step without further purification. LC/MS (ESI) (m/z): 349 (M+H)⁺.

Step 6: methyl (1S,3S,5S)-5-methyl-2-((4-(4-(S-methylsulfonimidoyl)phenoxy)benzoyl)-glycyl)-2-azabicyclo[3.1.0]hexane-3-carboxylate (J). To a mixture of compound H (117 mg, 0.34 mmol) and compound I (78 mg, 0.51 mmol) in DMF (5 mL) was added DIPEA (0.28 mL, 1.7 mmol), EDCI (129 mg, 0.68 mmol) and HOBT (68 mg, 0.51 mmol) at 0° C. under N2 atmosphere. The reaction was stirred at room temperature for 16 hours and diluted with EtOAc. The mixture was washed with saturated aq.NH₄Cl solution and brine and the organic layer was dried over anhydrous Na₂SO₄, filtered and concentrated to dryness under reduced pressure. The residue was purified by column chromatography on silica gel (DCM:MeOH=94:6) to give compound J (83 mg, yield 50.9%) as a light-yellow solid. LC/MS (ESI) m/z: 486(M+H)⁺.

Step 7: (1S,3S,5S)-5-methyl-2-((4-(4-(S-methylsulfonimidoyl)phenoxy)benzoyl)glycyl)-2-azabicyclo[3.1.0]hexane-3-carboxylic acid (K). To a solution of compound J (83 mg, 0.17 mmol) in MeOH (5 mL) and water (1 mL) was added a solution of LiOH·H₂O (22 mg, 0.51 mmol) at 0° C. and the mixture was stirred at room temperature for 2 hours. The mixture was acidified with 1N aq. HCl to pH˜3 and concentrated to dryness under reduced pressure to give compound K (79 mg, yield 97.5%) as a yellow solid, which was used directly in next step without further purification. LC/MS (ESI) (m/z): 472 (M+H)⁺.

Step 8: (1S,3S,5S)-N-((4-carbamimidoylthiophen-2-yl)methyl)-5-methyl-2-((4-(4-(S-methyl-sulfonimidoyl)phenoxy)benzoyl)glycyl)-2-azabicyclo[3.1.0]hexane-3-carboxamide (Compound 3). To a mixture of compound K (80 mg, 0.17 mmol) and compound L (40 mg, 0.26 mmol) in DMF (5 mL) was added DIPEA (0.14 mL, 0.85 mmol), EDCI (65 mg, 0.34 mmol) and HOBT (46 mg, 0.34 mmol) at 0° C. under N₂ atmosphere. The reaction was stirred at room temperature for 16 hours. The mixture was diluted with H₂O and extracted with CHCl₃/i-PrOH(3/1). The organic layer was dried over anhydrous Na₂SO₄, filtered and concentrated to dryness under reduced pressure. The residue was purified by column chromatography on silica gel (DCM:MeOH=4:1) and further purified by prep-HPLC to give Compound 3 (2.3 mg, yield 2.2%) as a white solid. ¹H NMR (400 MHz, CD₃OD) δ 8.53 (s, 1H), 8.21 (d, J=1.6 Hz, 1H), 8.05-7.99 (m, 2H), 7.93 (d, J=8.7 Hz, 2H), 7.41 (s, 1H), 7.24-7.19 (m, 2H), 7.18-7.13 (m, 2H), 4.58 (s, 1H), 4.55 (d, J=3.0 Hz, 2H), 4.36 (q, J=16.5 Hz, 2H), 3.42 (dd, J=6.0, 2.4 Hz, 1H), 3.16 (s, 4H), 2.42 (t, J=13.0 Hz, 1H), 2.17 (dd, J=13.4, 3.3 Hz, 1H), 1.30 (s, 3H), 1.15 (dd, J=5.6, 2.5 Hz, 1H), 0.81 (t, J=5.4 Hz, 1H). LC/MS (ESI) m/z: 609 (M+H)⁺.

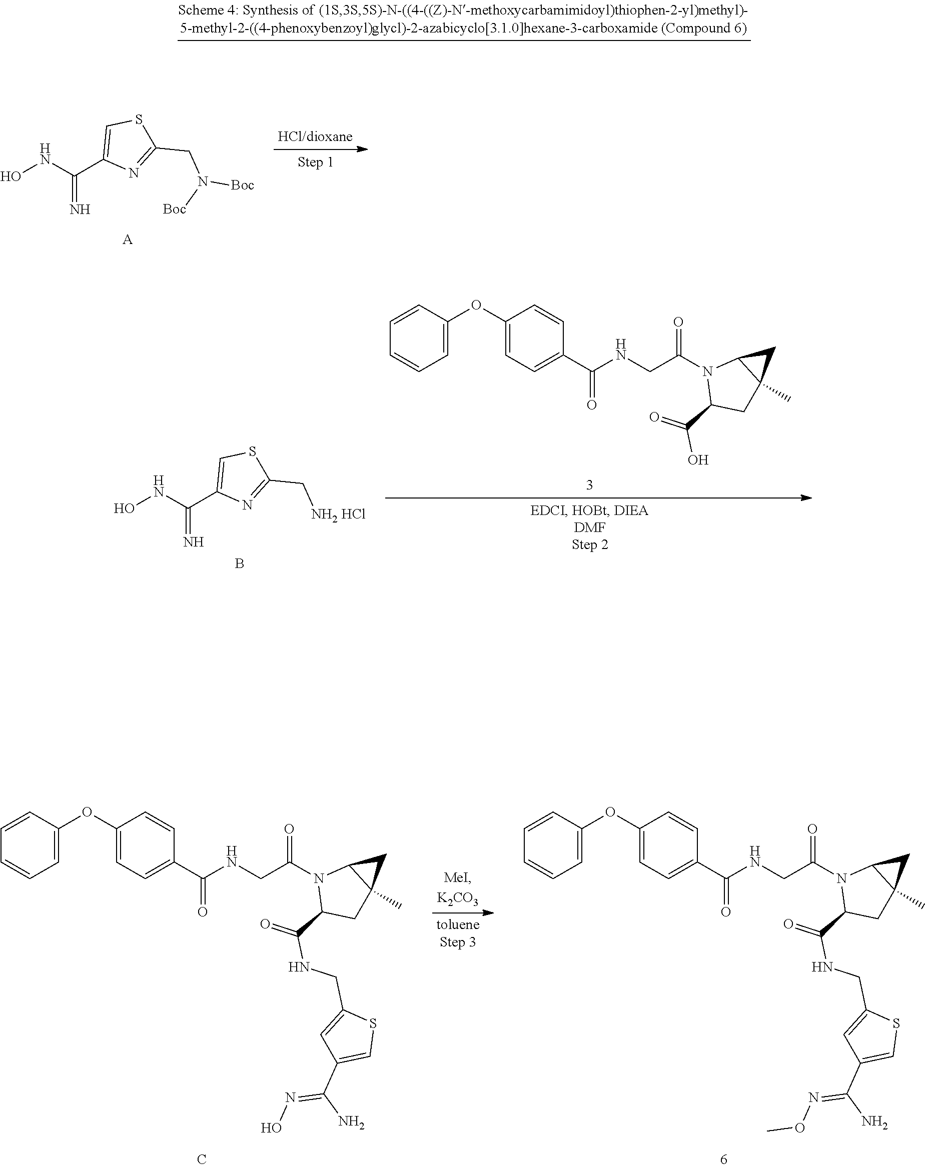

Step 1: 5-(aminomethyl)-N-hydroxythiophene-3-carboximidamide hydrochloride (B). A mixture of compound A (300 mg, 0.81 mmol) in HCl/1,4-dioxane (3 mL, 4M) was stirred at room temperature for 2 hours. The reaction mixture was concentrated to dryness, dissolved in DCM and concentrated to dryness again under vacuum to give compound B (167 mg, yield 99.8%) as a yellow solid, which was used directly in the next step without further purification. LC/MS (ESI) m/z: 172 (M+H)⁺.

Step 2: (1S,3S,5S)-N-((4-((Z)-N′-hydroxycarbamimidoyl)thiophen-2-yl)methyl)-5-methyl-2-((4-phenoxybenzoyl)glycyl)-2-azabicyclo[3.1.0]hexane-3-carboxamide (C). To a mixture of compound B (115 mg, 0.20 mmol) and compound 3 (50 mg, 0.13 mmol) in DMF (3 mL) was added DIPEA (82 mg, 0.63 mmol) at 0° C., followed by addition of EDCI (44 mg, 0.23 mmol) and HOBt (26 mg, 0.19 mmol). The resulting mixture was stirred at room temperature overnight. The mixture was diluted with EtOAc and washed with saturated aq. NH₄Cl solution and brine, dried over anhydrous Na₂SO₄, filtered and concentrated to dryness under reduced pressure. The residue was purified by prep-HPLC to give compound C (5.3 mg, yield 7.6%) as white solid. ¹H NMR (400 MHz, DMSO-d₆) δ 9.47 (s, 1H), 8.67 (t, J=5.8 Hz, 1H), 8.46 (t, J=5.8 Hz, 1H), 7.90 (d, J=8.8 Hz, 2H), 7.64 (d, J=1.6 Hz, 1H), 7.47-7.43 (m, 2H), 7.24-7.20 (m, 1H), 7.16 (s, 1H), 7.11-7.09 (m, 2H), 7.06-7.03 (m, 2H), 5.71 (s, 2H), 4.65 (dd, J=11.2, 11.2 Hz, 1H), 4.43-4.32 (m, 3H), 4.04-3.98 (m, 1H), 3.52-3.51 (m, 1H), 2.28 (t, J=12.4 Hz, 1H), 1.96 (dd, J=13.2, 13.2 Hz, 1H), 1.23 (s, 3H), 1.21-1.20 (m, 1H), 0.66 (t, J=5.2 Hz, 1H). LC/MS (ESI) m/z: 548 (M+H)⁺.

Step 3: (1S,3S,5S)-N-((4-((Z)-N′-methoxycarbamimidoyl)thiophen-2-yl)methyl)-5-methyl-2-((4-phenoxybenzoyl)glycyl)-2-azabicyclo[3.1.0]hexane-3-carboxamide (Compound 6). To a solution of compound C (37 mg, 0.068 mmol) in toluene (3 mL) was added K₂CO₃ (37 mg, 0.27 mmol), 18-crown-6 (2.0 mg, 0.007 mmol) and Mel (10 mg, 0.068 mmol) at 0° C. under N2 atmosphere. The mixture was stirred at 110° C. overnight. The mixture was diluted with H₂O and extracted with EtOAc twice. The combined organic layers were washed with brine, dried over anhydrous Na₂SO₄, filtered and concentrated to dryness under reduced pressure. The residue was purified by prep-TLC (DCM:MeOH=20:1) to give Compound 6 (2.4 mg, yield 6.3%) as white solid. ¹H NMR (400 MHz, CDCl₃) δ 7.79 (d, J=8.8 Hz, 2H), 7.43 (t, J=5.8 Hz, 1H), 7.40-7.36 (m, 2H), 7.33 (d, J=1.6 Hz, 1H), 7.20-7.16 (m, 2H), 7.11-7.07 (m, 1H), 7.07-7.03 (m, 2H), 7.02-6.99 (m, 2H), 4.87 (dd, J=10.4, 10.4 Hz, 1H), 4.71 (s, 1H), 4.59-4.54 (m, 1H), 4.51-4.45 (m, 1H), 4.38 (dd, J=10.0, 10.0 Hz, 1H), 3.84 (s, 3H), 3.09 (dd, J=6.4, 6.4 Hz, 1H), 2.72 (dd, J=12.8, 12.8 Hz, 1H), 2.10-2.04 (m, 1H), 1.31 (s, 3H), 1.09 (dd, J=6.4, 6.4 Hz, 1H), 0.75 (t, J=6.0 Hz, 1H). LC/MS (ESI) m/z: 562 (M+H)⁺.

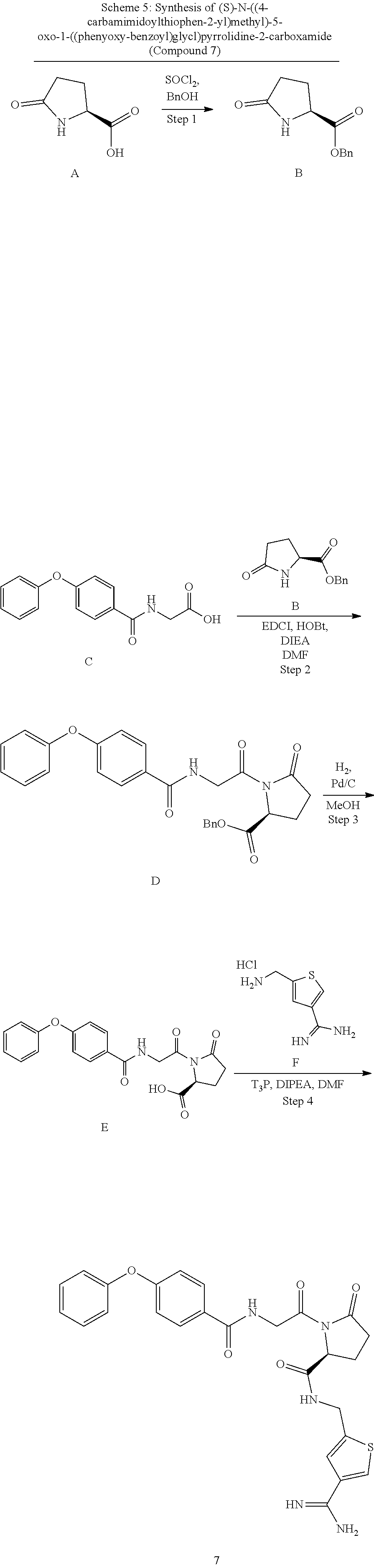

Step 1: benzyl (S)-5-oxopyrrolidine-2-carboxylate (B). To a solution of compound A (2.5 g, 19.4 mmol) in benzyl alcohol (20 mL) was added SOCl₂ (2.8 mL, 38.8 mmol) at 0° C. under N2 atmosphere. The reaction was stirred at room temperature overnight. The reaction was concentrated to dryness under reduced pressure and the residue was purified by column chromatography on silica gel (Hexane:EtOAc=1:2) to give compound B (2.8 g, 66% yield) as a colorless oil. LC/MS (ESI) m/z: 220 (M+H)⁺.

Step 2: benzyl (S)-5-oxo-1-((4-phenoxybenzoyl)glycyl)pyrrolidine-2-carboxylate (D). To a solution of compound C (500 mg, 1.84 mmol) in DMF (5 mL) was added DIPEA (712.8 mg, 5.5 mmol), compound B (613.2 mg, 2.8 mmol), HOBt (322.9 mg, 2.4 mmol) and EDCI (493.8 mg, 2.5 mmol) at room temperature under N₂ atmosphere. The reaction was stirred at room temperature overnight and quenched with saturated aqueous NH₄Cl. The resulting mixture was extracted with EtOAc twice. The combined organic layers were washed with H₂O and brine (2×100 mL), dried over anhydrous Na₂SO₄, filtered and concentrated to dryness under reduced pressure. The residue was purified by column chromatography on silica gel (hexane:EtOAc=1:1) to give compound D (110 mg, 13% yield) as a light yellow oil. LC/MS (ESI) m/z: 473 (M+H)⁺.

Step 3: (S)-5-oxo-1-((4-phenoxybenzoyl)glycyl)pyrrolidine-2-carboxylic acid (E). To a solution of compound D (110 mg, 0.23 mmol) in MeOH was added 10% Pd/C (20 mg) at room temperature under N₂ atmosphere and the reaction mixture was stirred at room temperature under H₂ atmosphere for 2 hours. The mixture was filtered and concentrated to dryness under reduced pressure to give compound E (71 mg, 80% yield) as a colorless oil, which was used directly in the next step without further purification. LC/MS (ESI) m/z: 483 (M+H)⁺.

Step 4: (S)-N-((4-carbamimidoylthiophen-2-yl)methyl)-5-oxo-1-((4-phenoxybenzoyl)glycyl)-pyrrolidine-2-carboxamide (Compound 7). To a mixture of compound E (71 mg, 0.183 mmol) and compound F (522 mg, 2.75 mmol) in DMF (3 mL) was added DIPEA (141.6 mg, 1.1 mmol) and T₃P (349 mg, 0.549 mmol, 50% wt in EtOAc) at room temperature under N2 atmosphere. The mixture was stirred at room temperature overnight and quenched with saturated aqueous NH₄Cl. The resulting mixture was extracted with EtOAc twice. The combined organic layers were washed with H₂O and brine, dried over anhydrous Na₂SO₄, filtered and concentrated to dryness. The residue was purified by prep-HPLC to give Compound 7 (3 mg, 3% yield) as a white solid. ¹H NMR (400 MHz, DMSO-d₆) δ 11.00 (s, 1H), 9.03 (t, J=5.7 Hz, 1H), 8.75-8.69 (m, 1H), 8.38-8.34 (m, 1H), 7.92-7.89 (m, 2H), 7.47-7.42 (m, 3H), 7.24-7.21 (m, 1H), 7.11-7.09 (m, 2H), 7.07-7.04 (m, 2H), 4.66-4.61 (m, 2H), 4.54-4.49 (m, 1H), 4.45-4.34 (m, 2H), 2.69-2.55 (m, 2H), 2.46-2.31 (m, 1H), 2.01-1.87 (m, 1H). LC/MS (ESI) m/z: 520 (M+H)⁺.

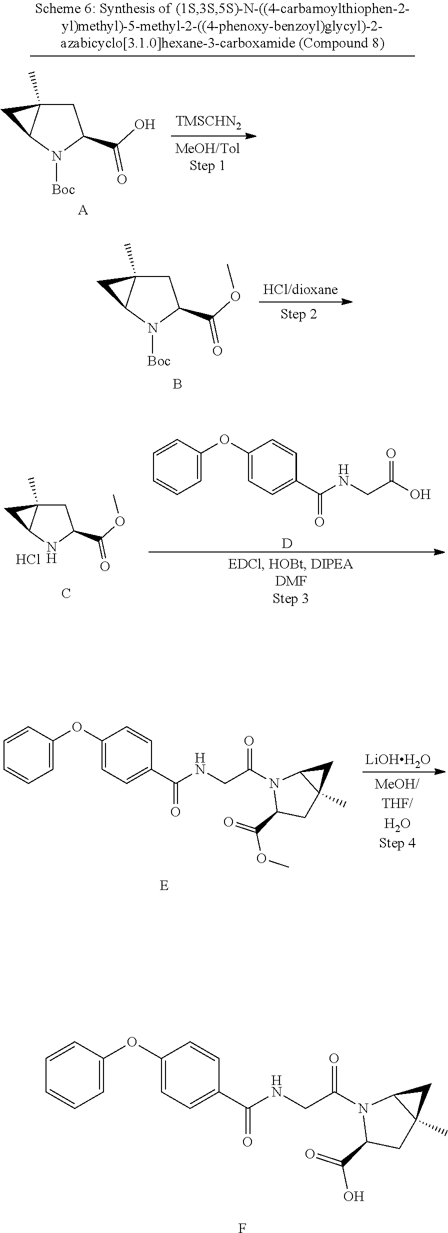

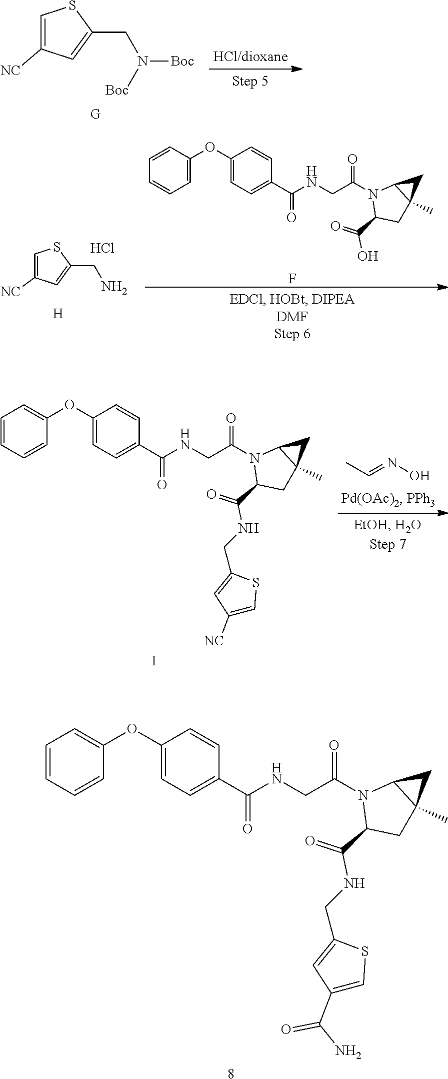

Step 1: 2-(tert-butyl)-3-methyl-(1S,3,5)-5-methyl-2-azabicyclo[3.1.0]hexane-2,3-dicarboxylate (B). A solution of compound A (100 mg, 0.41 mmol) in MeOH (1 ml) and toluene (2.5 mL) was added TMSCHN₂ (0.41 mL, 0.84 mmol, 2 M) dropwise at 0° C. under N₂ atmosphere. The reaction was stirred at room temperature for 2 hours. The reaction mixture was quenched with AcOH and concentrated to dryness under vacuum. The residue was purified by column chromatography on silica gel (PE:EtOAc=50:1 to 3:1) to give compound B (100 mg, yield 94.5%) as an oil. LC/MS (ESI) m/z: 256 (M+H)⁺.

Step 2: methyl (1S,3S,52)-5-methyl-2-azabicyclo[3.1.]hexane-3-carboxylate hydrochloride (C). A mixture of compound B (100 mg, 0.41 mmol) in HCl/1,4-dioxane (2 mL, 4M) was stirred at room temperature for 2 hours. The reaction mixture was washed with ether and dried over anhydrous Na₂SO₄, filtered, and concentrated to dryness under vacuum to give compound C (75 mg, yield 99.9%) as a colorless oil, which was used directly in the next step without further purification. LC/MS (ESI) m/z: 156 (M+H)⁺.

Step 3: methyl (1S,3S,5S)-5-methyl-2-((4-phenoxybenzoyl)glycyl)-2-azabicyclo[3.1.0]-hexane-3-carboxylate (E). To a mixture of compound C (75 mg, 0.39 mmol) and compound D (106 mg, 0.39 mmol) in DMF (3 mL) was added DIPEA (252 mg, 1.95 mmol) at 0° C., followed by addition of EDCI (134 mg, 0.70 mmol), HOBt (79 mg, 0.59 mmol). The resulting mixture was stirred at room temperature overnight. The mixture was diluted with EtOAc and washed with saturated aq.NH₄Cl solution and brine, dried over anhydrous Na₂SO₄, filtered and concentrated to dryness under reduced pressure. The residue was purified by column chromatography on silica gel (PE:EtOAc=10:1 to 2:1) to give compound E (120 mg, yield 75.1%) as a yellow oil. LC/MS (ESI) m/z: 409 (M+H)⁺.

Step 4: (1S,3S,5S)-5-methyl-2-((4-phenoxybenzoyl)glycyl)-2-azabicyclo[3.1.0]hexane-3-carboxylic acid (F). To a solution of compound E (120 mg, 0.29 mmol) in THE (1 mL) and MeOH (2 mL) was added a solution of lithium hydroxide (59 mg, 1.47 mmol) in water (1 mL) at 0° C. The mixture was stirred at room temperature for 1.5 hours. The mixture was diluted with water and washed with EtOAc twice. The aqueous layer was acidified with 0.5 M aq. HCl solution to pH-5 and extracted with EtOAc twice. The combined organic layers were washed with brine, dried over anhydrous Na₂SO₄, filtered and concentrated to dryness under reduced pressure to give compound F (115 mg, yield 99.3%) as white solid. LC/MS (ESI) m/z: 395 (M+H)⁺.

Step 5: 5-(aminomethyl)thiophene-3-carbonitrile hydrochloride (H). A mixture of compound G (500 mg, 1.48 mmol) in HCl/1,4-dioxane (5 mL, 4M) was stirred at room temperature for 1.5 hours. The reaction mixture was concentrated to dryness, washed with DCM and dried under vacuum to give compound H (200 mg, yield 98.0%) as a yellow solid, which was used directly in the next step without further purification. LC/MS (ESI) m/z: 139 (M+H)⁺.

Step 6: (1S,3S,5S)-N-((4-cyanothiophen-2-yl)methyl)-5-methyl-2-((4-phenoxybenzoyl)-glycyl)-2-azabicyclo[3.1.0]hexane-3-carboxamide (1). To a mixture of compound F (115 mg, 0.29 mmol) and compound H (75 mg, 0.44 mmol) in DMF (3 mL) was added DIPEA (187 mg, 1.45 mmol) at 0° C., followed by addition of EDCI (100 mg, 0.52 mmol) and HOBt (59 mg, 0.44 mmol). The resulting mixture was stirred at room temperature overnight. The mixture was diluted with EtOAc and washed with saturated aq.NH₄Cl solution and brine, dried over anhydrous Na₂SO₄, filtered and concentrated to dryness under reduced pressure. The residue was purified by prep-HPLC to give compound I (2.3 mg, yield 1.5%) as white solid. ¹H NMR (400 MHz, DMSO-d₆) δ 8.70 (t, J=5.6 Hz, 1H), 8.57 (t, J=6.0 Hz, 1H), 8.39 (d, J=1.2 Hz, 1H), 7.91-7.88 (m, 2H), 7.47-7.43 (m, 2H), 7.30 (d, J=0.8 Hz, 1H), 7.22 (t, J=7.4 Hz, 1H), 7.10 (d, J=7.6 Hz, 2H), 7.05-7.02 (m, 2H), 4.66 (dd, J=11.2, 11.2 Hz, 1H), 4.43-4.38 (m, 2H), 4.37-4.30 (m, 1H), 4.03 (dd, J=16.8, 16.8 Hz, 1H), 3.59-3.48 (m, 1H), 2.30 (t, J=12.4 Hz, 1H), 1.97 (dd, J=13.6, 13.6 Hz, 1H), 1.23 (s, 3H), 1.17 (dd, J=4.8, 4.8 Hz, 1H), 0.68 (t, J=5.4 Hz, 1H). LC/MS (ESI) m/z: 515 (M+H)⁺.

Step 8: (1S,3S,5S)-N-((4-carbamoylthiophen-2-yl)methyl)-5-methyl-2-((4-phenoxybenzoyl)-glycyl)-2-azabicyclo[3.1.0]hexane-3-carboxamide (Compound 8). To a solution of compound I (50 mg, 0.097 mmol) in EtOH (2 mL) and H₂O (0.5 mL) was added (E)-acetaldehyde oxime (13 mg, 0.19 mmol), Pd(OAc)₂ (1 mg, 0.005 mmol) and PPh₃ (2.5 mg, 0.09 mmol) at 0° C. under N2 atmosphere. The mixture was stirred at 80° C. for 16 hours. The mixture was quenched with H₂O and extracted with CHCl₃/i-PrOH(v:v=3/1). The organic layers were dried over anhydrous Na₂SO₄, filtered and concentrated to dryness under reduced pressure. The residue was purified by column chromatography on silica gel (DCM:MeOH=15:1) and further purified by prep-HPLC to give Compound 8 (12.9 mg, yield 24.8%) as a white solid. ¹H NMR (400 MHz, CD₃OD) δ 7.93 (d, J=1.5 Hz, 1H), 7.84 (d, J=2.0 Hz, 1H), 7.83 (d, J=2.0 Hz, 1H), 7.41 (dd, J=8.4, 7.6 Hz, 2H), 7.36 (d, J=1.2 Hz, 1H), 7.20 (t, J=7.4 Hz, 1H), 7.06 (dd, J=8.6, 0.9 Hz, 2H), 7.01 (t, J=2.4 Hz, 1H), 6.99 (d, J=2.2 Hz, 1H), 4.82 (dd, J=11.4, 3.3 Hz, 1H), 4.50 (s, 2H), 4.40 (d, J=16.5 Hz, 1H), 4.27 (d, J=16.5 Hz, 1H), 3.38 (dd, J=6.0, 2.4 Hz, 1H), 2.40 (dd, J=18.3, 6.5 Hz, 1H), 2.16 (dd, J=3.4, 3.3 Hz, 1H), 1.28 (s, 3H), 1.15 (dd, J=5.4, 2.0 Hz, 1H), 0.77 (d, J=5.9 Hz, 1H). LC/MS (ESI) m/z: 533 (M+H)⁺.

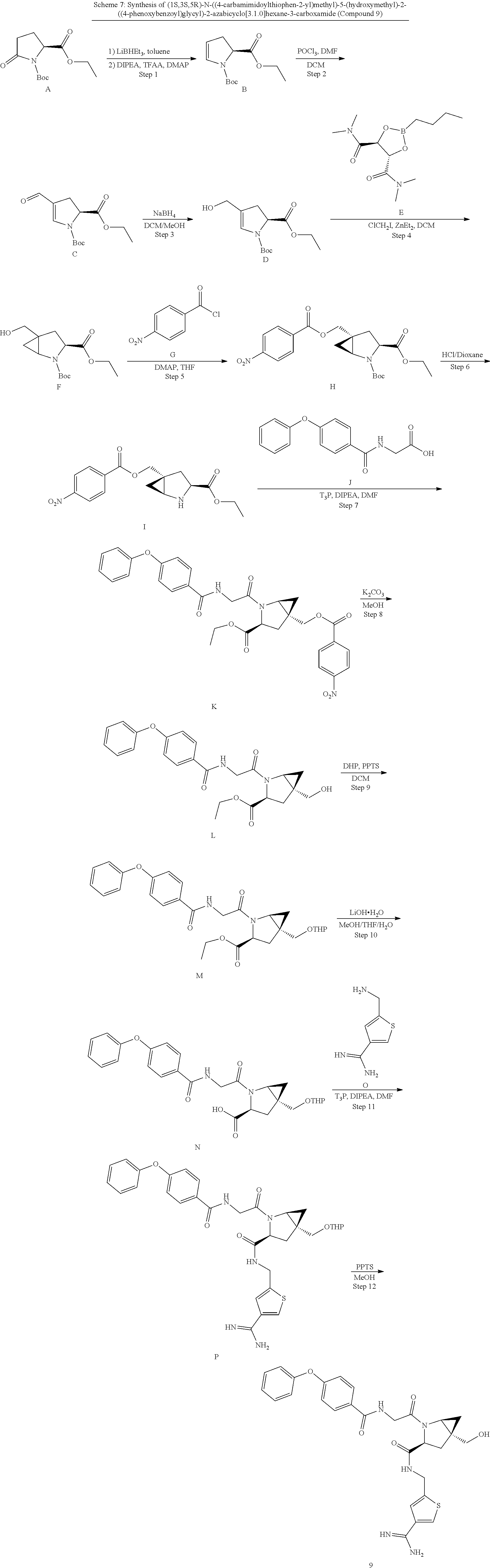

Step 1: 1-(tert-butyl) 2-ethyl (S)-2,3-dihydro-1H-pyrrole-1,2-dicarboxylate (B). To a solution of compound A (50 g, 194.34 mmol) in toluene (389 mL) was added LiBHEt₃ (206 mL, 204.05 mmol) dropwise at −78° C. under N₂ atmosphere and the mixture was stirred at −78° C. for 1 hour. DIPEA (193.5 mL, 1.11 mol), DMAP (0.475 g, 3.89 mmol) and TFAA (33 mL, 233 mmol) were added to the mixture at −78° C. under N2 atmosphere. The reaction mixture was stirred at 25° C. for 2 hours. The mixture was quenched with ice water and organic layer was separated. The organic layers were washed with water, dried over anhydrous Na₂SO₄, filtered and concentrated to dryness under reduced pressure. The residue was purified by column chromatography on silica gel (PE:EtOAc=100:1 to 20:1) to give compound B (38.5 g, yield 82%) as a yellow oil. LC/MS (ESI) (m/z): 242 (M+H)⁺.

Step 2: 1-(tert-butyl) 2-ethyl (S)-4-formyl-2,3-dihydro-1H-pyrrole-1,2-dicarboxylate (C). A mixture of DMF (49.3 mL, 639 mmol) and POCl₃ (29.8 mL, 320 mmol) was stirred at 0° C. under N2 atmosphere for 30 minutes. The mixture was diluted with DCM (360 mL) and a solution of compound B (38.5 g, 160 mmol) in DCM (50 mL) was added at 0° C. The reaction was stirred at 25° C. for 1.5 hours and quenched with 2 M NaOH solution. EtOAc was added and the mixture was separated. The organic layer was washed with brine, dried over anhydrous Na₂SO₄, filtered and concentrated to dryness under reduced pressure. The residue was purified by column chromatography on silica gel (PE:EtOAc=3:1) to give compound C (39 g, yield 92%) as a yellow oil. LC/MS (ESI) (m/z): 270 (M+H)⁺.

Step 3: 1-(tert-butyl) 2-ethyl (S)-4-(hydroxymethyl)-2,3-dihydro-1H-pyrrole-1,2-dicarboxylate (D). To a solution of compound C (39 g, 145 mmol) in DCM (400 mL) and methanol (200 mL) was added NaBH₄ (8.22 g, 217 mmol) in portions at −70° C. under N2 atmosphere and the mixture was stirred at 0° C. for half an hour. The mixture was quenched with aq. NH₄Cl and extracted with DCM twice. The combined organic layers were washed with brine, dried over anhydrous Na₂SO₄, filtered and concentrated to dryness under reduced pressure. The residue was purified by column chromatography on silica gel (PE:EtOAc=1:1) to give compound D (33.6 g, yield 85.5%) as a light yellow oil. LC/MS (ESI) m/z: 272 (M+H)⁺.

Step 4: 2-(tert-butyl) 3-ethyl (3S,5R)-5-(hydroxymethyl)-2-azabicyclo[3.1.0]hexane-2,3-dicarboxylate (F). To a solution of ZnEt₂ (46 mL, 46.1 mmol) in DCM (150 mL) was added ClCH₂I (16.3 g, 92.3 mmol) dropwise at −20° C. under N2 atmosphere and the mixture was stirred at −20° C. for half an hour. A mixture of compound E (7.5 g, 27.7 mmol) and compound D (5 g, 18.5 mmol) in DCM were dropwise added at −20° C. and the mixture was stirred at −20° C. for 12 hours. The reaction was quenched with cold aq.NH₄Cl solution and extracted with EtOAc twice. The combined organic layers were washed with brine, dried over anhydrous Na₂SO₄, filtered and concentrated to dryness under reduced pressure. The residue was purified by column chromatography on silica gel (PE:EtOAc=3:1) to give compound F (4.07 g, yield 77.5%) as a colorless oil. LC/MS (ESI) m/z: 286 (M+H)⁺.

Step 5: 2-(tert-butyl) 3-ethyl (1S,3S,5R)-5-(((4-nitrobenzoyl)oxy)methyl)-2-azabicyclo-[3.1.0]hexane-2,3-dicarboxylate (H). To a solution of compound F (4.07 g, 14.26 mmol) in THE (100 mL) was added compound G (3.44 g, 18.54 mmol) and DMAP (5.23 g, 42.79 mmol) at 0° C. and the mixture was stirred at 40° C. for 16 hours in a microwave reactor. The reaction was cooled down to 25° C. and poured into iced-water. The mixture was extracted with EtOAc twice. The combined organic layers were washed with brine, dried over anhydrous Na₂SO₄, filtered and concentrated to dryness under reduced pressure. The residue was purified by SFC to give compound H (2.25 g, yield 36.3%) as a colorless oil. LC/MS (ESI) (m/z): 435 (M+H)⁺.

Step 6: ethyl (1S,3S,5R)-5-(((4-nitrobenzoyl)oxy)methyl)-2-azabicyclo[3.1.0]hexane-3-carboxylate (1). A mixture of compound H (0.2 g, 0.46 mmol) and HCl/1,4-dioxane (5 mL) was stirred at room temperature for 1 hour. The reaction mixture was concentrated to dryness under reduced pressure, diluted with DCM again and dried under vacuum to give compound I (0.15 g, yield 97.5%) as yellow oil, which was used directly in the next step without further purification. LC/MS (ESI) m/z: 335 (M+H)⁺.

Step 7: ethyl (1S,3S,5R)-5-(((4-nitrobenzoyl)oxy)methyl)-2-((4-phenoxybenzoyl)glycyl)-2-azabicyclo[3.1.0]hexane-3-carboxylate (K). To a mixture of compound I (0.15 g, 0.45 mmol) and compound J (0.134 g, 0.49 mmol) in DMF (5 mL) was added DIPEA (0.39 mL, 2.24 mmol) and T₃P (0.714 g, 1.13 mmol) at 0° C. under N2 atmosphere and the mixture was stirred at 25° C. for 16 hours. The mixture was diluted with EtOAc and washed with saturated aq. NH₄Cl solution and brine. The organic layer was dried over anhydrous Na₂SO₄, filtered and concentrated to dryness under reduced pressure. The residue was purified by column chromatography on silica gel (PE:EtOAc=2:3) to give compound K (0.17 g, yield 64.9%) as a white solid. LC/MS (ESI) (m/z): 588 (M+H)⁺.

Step 8: methyl (1S,3S,5R)-5-(hydroxymethyl)-2-((4-phenoxybenzoyl)glycyl)-2-azabicyclo-[3.1.0]hexane-3-carboxylate (9). To a solution of compound K (171 mg, 0.29 mmol) in MeOH (5 mL) was added K₂CO₃ (80 mg, 0.58 mmol) and the mixture was stirred at 25° C. for 1 hour. The mixture was diluted with EtOAc and washed with brine. The organic layer was dried over anhydrous Na₂SO₄, filtered and concentrated to dryness under reduced pressure. The residue was purified by column chromatography on silica gel (DCM:MeOH=95:5) to give compound L (123 mg, yield 96.8%) as a white solid. LC/MS (ESI) (m/z): 425 (M+H)⁺.

Step 9: methyl (1S,3S,5R)-2-((4-phenoxybenzoyl)glycyl)-5-(((tetrahydro-2H-pyran-2-yl)oxy)-methyl)-2-azabicyclo[3.1.0]hexane-3-carboxylate (M). To a solution of compound L (0.11 g, 0.25 mmol) in DCM (5 mL) was added DHP (0.03 mL, 0.33 mmol) and PPTS (19 mg, 0.08 mmol) at 0° C. and the mixture was stirred at 25° C. for 16 hour. The mixture was diluted with EtOAc and washed with brine. The organic layer was dried over anhydrous Na₂SO₄, filtered and concentrated to dryness under reduced pressure. The residue was purified by column chromatography on silica gel (DCM:MeOH=96:4) to give compound M (118 mg, yield 92.2%) as a yellow oil. LC/MS (ESI) m/z: 509 (M+H)⁺.

Step 10: (1S,3S,5R)-2-((4-phenoxybenzoyl)glycyl)-5-(((tetrahydro-2H-pyran-2-yl)oxy)-methyl)-2-azabicyclo[3.1.0]hexane-3-carboxylic acid (11). To a solution of compound M (118 mg, 0.23 mmol) in MeOH (5 mL) and water (1 mL) was added a solution of LiOH·H₂O (10 mg, 0.23 mmol) at 0° C. and the mixture was stirred at room temperature for 16 hours. The mixture was acidified with 1N aq.HCl to pH-3 and concentrated to dryness under reduced pressure to give compound N (108 mg, yield 94.7%) as a yellow semi-solid, which was used directly in next step without further purification. LC/MS (ESI) (m/z): 495 (M+H)⁺.

Step 11: (1S,3S,5R)-N-((4-carbamimidoylthiophen-2-yl)methyl)-2-((4-phenoxybenzoyl)-glycyl)-5-(((tetrahydro-2H-pyran-2-yl)oxy)methyl)-2-azabicyclo[3.1.0]hexane-3-carboxamide (12). To a mixture of compound N (136 mg, 0.28 mmol) and O (640 mg, 0.42 mmol) in DMF (5 mL) was added DIPEA (0.27 mL, 1.68 mmol) and T₃P (0.53 g, 0.84 mmol) at 0° C. under N2 atmosphere and the mixture was stirred at 25° C. for 16 hours. The mixture was diluted with EtOAc and washed with saturated aq. NH₄Cl solution and brine. The organic layer was dried over anhydrous Na₂SO₄, filtered and concentrated to dryness under reduced pressure. The residue was purified by column chromatography on silica gel (DCM:MeOH=10:1) to give compound P (71 mg, yield 41.2%) as a colorless oil. LC/MS (ESI) (m/z): 632 (M+H)⁺.

Step 12: (1S,3S,5R)-N-((4-carbamimidoylthiophen-2-yl)methyl)-5-(hydroxymethyl)-2-((4-phenoxybenzoyl)glycyl)-2-azabicyclo[3.1.0]hexane-3-carboxamide (Compound 9). To a solution of compound P (60 mg, 0.1 mmol) in MeOH (4 mL) was added PPTS (12 mg, 0.05 mmol) and the mixture was stirred at room temperature for 16 hours. The mixture was diluted with H₂O and extracted with CHCl₃/i-PrOH(v:v=3/1). The organic layer was dried over anhydrous Na₂SO₄, filtered and concentrated to dryness under reduced pressure. The residue was purified by column chromatography on silica gel (DCM:MeOH=9:1) and further purified by prep-HPLC to give Compound 9 (2.4 mg, yield 4.6%) as a white solid. ¹H NMR (400 MHz, CD₃OD) δ 7.93 (d, J=1.5 Hz, 1H), 7.84 (d, J=2.0 Hz, 1H), 7.83 (d, J=2.0 Hz, 1H), 7.41 (dd, J=8.4, 7.6 Hz, 2H), 7.36 (d, J=1.2 Hz, 1H), 7.20 (t, J=7.4 Hz, 1H), 7.06 (dd, J=8.6, 0.9 Hz, 2H), 7.01 (t, J=2.4 Hz, 1H), 6.99 (d, J=2.2 Hz, 1H), 4.82 (dd, J=11.4, 3.3 Hz, 1H), 4.50 (s, 2H), 4.40 (d, J=16.5 Hz, 1H), 4.27 (d, J=16.5 Hz, 1H), 3.38 (dd, J=6.0, 2.4 Hz, 1H), 2.40 (dd, J=18.3, 6.5 Hz, 1H), 2.16 (dd, J=13.4, 3.3 Hz, 1H), 1.28 (s, 3H), 1.15 (dd, J=5.4, 2.0 Hz, 1H), 0.77 (d, J=5.9 Hz, 1H). LC/MS (ESI) m/z: 548(M+H)⁺.

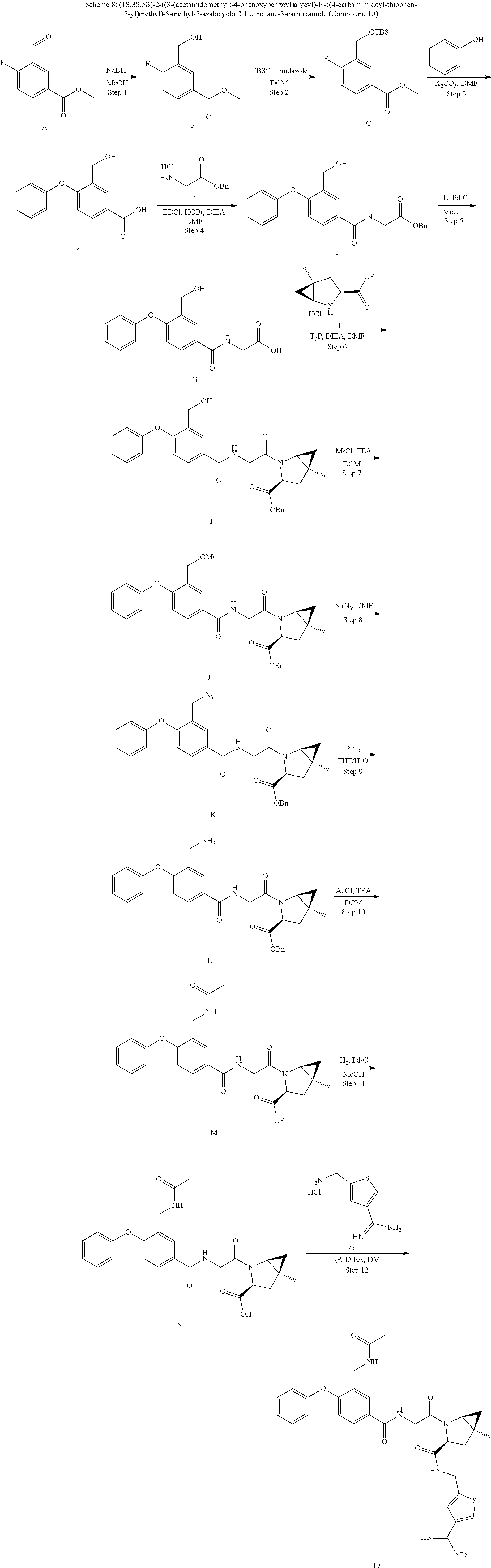

Step 1: methyl 4-fluoro-3-(hydroxymethyl)benzoate (B). To a solution of compound A (2 g, 11 mmol) in MeOH (20 mL) was added NaBH₄ (454 mg, 12 mmol) at 0° C. under N2 atmosphere and the reaction was stirred at room temperature for 1 hour. The reaction was quenched with ice-water and the mixture was extracted with DCM twice. The combined organic layers were washed with brine, dried over anhydrous Na₂SO₄, filtered and concentrated to dryness under reduced pressure to give compound B (2 g, yield 98.8%) as a colorless oil, which was used directly in the next step without further purification. LC/MS (ESI) m/z: 299 (M+H)⁺.

Step 2: methyl 3-(((tert-butyldimethylsilyl)oxy)methyl)-4-fluorobenzoate (C). To a solution of compound 2 (2 g, 10.8 mmol) in DCM (20 mL) was added imidazole (817 mg, 12 mmol) and TBSCl (1.8 g, 12 mmol) at room temperature under N2 atmosphere. The mixture was stirred at room temperature overnight and quenched with saturated aqueous NH₄Cl. The resulting mixture was extracted with EtOAc twice. The combined organic layers were washed with brine, dried over anhydrous Na₂SO₄, filtered and concentrated to dryness under reduced pressure. The residue was purified by column chromatography on silica gel (PE:EtOAc=100:1) to give compound C (2 g, yield 13%) as a light-yellow oil. LC/MS (ESI) m/z: 299 (M+H)⁺.

Step 3: 3-(hydroxymethyl)-4-phenoxybenzoic acid (D). To a solution of compound C (2 g, 6.71 mmol) in DMF (20 mL) was added Cs₂CO₃ (4.3 g, 13.4 mmol) and phenol (694 mg, 7.4 mmol) at room temperature under N2 atmosphere. The mixture was stirred at 120° C. overnight. The reaction was cooled down to room temperature and quenched with saturated aqueous NH₄Cl. The resulting mixture was extracted with EtOAc twice. The combined organic layers were washed with brine, dried over anhydrous Na₂SO₄, filtered and concentrated to dryness under reduced pressure. The residue was purified by column chromatography on silica gel (DCM:MeOH=15:1) to give compound D (430 mg, yield 26%) as a light-yellow oil. LC/MS (ESI) m/z: 243 (M−H)⁻.

Step 4: benzyl (3-(hydroxymethyl)-4-phenoxybenzoyl)glycinate (F). To a mixture of compound D (105 mg, 0.43 mmol) and compound E (130 mg, 0.64 mmol) in DMF (3 mL) was added DIPEA (333 mg, 2.58 mmol), HOBt (75 mg, 0.56 mmol) and EDCI (115 mg, 0.60 mmol) at room temperature under N2 atmosphere. The mixture was stirred at room temperature overnight and quenched with saturated aqueous NH₄Cl. The resulting mixture was extracted with EtOAc twice. The combined organic layers were washed with brine, dried over anhydrous Na₂SO₄, filtered and concentrated to dryness under reduced pressure. The residue was purified by column chromatography on silica gel (PE:EtOAc=1:1) to give compound F (130 mg, yield 77%) as a light-yellow oil. LC/MS (ESI) m/z: 392 (M+H)⁺.

Step 5: (3-(hydroxymethyl)-4-phenoxybenzoyl)glycine (G). To a stirred solution of compound F (130 mg, 0.33 mmol) in MeOH (2 mL) was added Pd/C (30 mg, 10% wt) at room temperature under N2 atmosphere and the reaction was stirred at room temperature under H₂ atmosphere for 2 hours. The mixture was filtered and concentrated to dryness under reduced pressure to give compound G (85 mg, yield 85%) as a colorless oil, which was used directly in the next step without further purification. LC/MS (ESI) m/z: 302 (M+H)⁺.

Step 6: benzyl (1S,3S,5S)-5-methyl-2-((3-(((methylsulfonyl)oxy)methyl)-4-phenoxy-benzoyl)-glycyl)-2-azabicyclo[3.1.0]hexane-3-carboxylate (1). To a mixture of compound G (85.0 mg, 0.27 mmol) and compound H (106 mg, 0.40 mmol) in DMF (3 mL) was added DIPEA (174.0 mg, 1.35 mmol) and T₃P (343 mg, 0.54 mmol, 50% in EtOAc) at room temperature under N2 atmosphere. The mixture was stirred at room temperature overnight and quenched with saturated aqueous NH₄Cl. The resulting mixture was extracted with EtOAc twice. The combined organic layers were washed with brine, dried over anhydrous Na₂SO₄, filtered and concentrated to dryness under reduced pressure. The residue was purified by column chromatography on silica gel (PE:EtOAc=1:1) to give compound I (110 mg, yield 76%) as a light-yellow oil. LC/MS (ESI) m/z: 515 (M+H)⁺.

Step 7: benzyl (1S,3S,5S)-5-methyl-2-((3-(((methylsulfonyl)oxy)methyl)-4-phenoxy-benzoyl)glycyl)-2-azabicyclo[3.1.0]hexane-3-carboxylate (J). To a solution of compound I (110 mg, 0.21 mmol) in DCM (3 mL) was added TEA (86.8 mg, 0.86 mmol) and MsCl (48.3 mg, 0.42 mmol) at 0° C. under N2 atmosphere and the mixture was stirred at 0° C. for 1 hour. The reaction was quenched with ice-water and the resulting mixture was extracted with DCM twice. The combined organic layers were washed with brine, dried over anhydrous Na₂SO₄, filtered and concentrated dryness under reduced pressure to give compound J (108 mg, yield 100%) as a colorless oil, which was used directly in the next step without further purification.

Step 8: benzyl (1S,3S,5S)-2-((3-(azidomethyl)-4-phenoxybenzoyl)glycyl)-5-methyl-2-azabicyclo[3.1.0]hexane-3-carboxylate (K). To a solution of compound J (108 mg, 0.21 mmol) in DMF (3 mL) was added NaN₃ (16.7 mg, 0.27 mmol) at room temperature under N2 atmosphere and the mixture was stirred at 40° C. overnight. The reaction was quenched with saturated aqueous NH₄Cl and the resulting mixture was extracted with EtOAc twice. The combined organic layers were washed with brine, dried over anhydrous Na₂SO₄, filtered and concentrated to dryness under reduced pressure. The residue was purified by column chromatography on silica gel (DCM:MeOH=20:1) to give compound K (100 mg, yield 87%) as a light-yellow oil. LC/MS (ESI) m/z: 540 (M+H)⁺.

Step 9: benzyl (1S,3S,5S)-2-((3-(aminomethyl)-4-phenoxybenzoyl)glycyl)-5-methyl-2-azabicyclo[3.1.0]hexane-3-carboxylate (L). To a solution of compound K (100 mg, 0.185 mmol) in THE (3 mL) and H₂O (0.3 mL) was added PPh₃ (145.4 mg, 0.555 mmol) at room temperature under N2 atmosphere and the mixture was stirred at room temperature overnight. The reaction was extracted with EtOAc twice. The combined organic layers were washed with brine, dried over anhydrous Na₂SO₄, filtered and concentrated to dryness under reduced pressure. The residue was purified by column chromatography on silica gel (DCM:MeOH=15:1) to give compound 10 (44 mg, yield 46%) as a light-yellow oil. LC/MS (ESI) m/z: 514 (M+H)⁺.

Step 10: benzyl (1S,3S,5S)-2-((3-(acetamidomethyl)-4-phenoxybenzoyl)glycyl)-5-methyl-2-azabicyclo[3.1.0]hexane-3-carboxylate (M). To a solution of compound L (44 mg, 0.086 mmol) in DCM (3 mL) was added TEA (25.9 mg, 0.257 mmol) and acetyl chloride (10.0 mg, 0.128 mmol) at 0° C. under N2 atmosphere. The mixture was stirred at room temperature for 1 hour and H₂O was added. The mixture was extracted with DCM and the combined organic layers were washed with brine, dried over Na₂SO₄, filtered and concentrated to dryness. The residue was purified by column chromatography on silica gel (DCM:MeOH=20:1) to give compound M (40 mg, yield 85%) as a light-yellow oil. LC/MS (ESI) m/z: 556 (M+H)⁺.

Step 11: (1S,3S,5S)-2-((3-(acetamidomethyl)-4-phenoxybenzoyl)glycyl)-5-methyl-2-azabicyclo[3.1.0]hexane-3-carboxylic acid (N). To a stirred solution of compound M (40 mg, 0.072 mmol) in MeOH (1 mL) was added Pd/C (20 mg, 10% wt) at room temperature under N₂ atmosphere and the reaction was stirred under H₂ atmosphere for 2 hours. The mixture was filtered and concentrated to dryness under reduced pressure to give compound N (25 mg, yield 76%) as colorless oil, which was used directly in the next step without further purification. LC/MS (ESI) m/z: 466 (M+H)⁺.

Step 12: (1S,3S,5S)-2-((3-(acetamidomethyl)-4-phenoxybenzoyl)glycyl)-5-methyl-2-azabicyclo[3.1.0]hexane-3-carboxylic acid (Compound 10). To a mixture of compound N (25 mg, 0.054 mmol) and compound O (15.4 mg, 0.081 mmol) in DMF (2 mL) was added DIPEA (35 mg, 0.27 mmol) and T₃P (68.7 mg, 0.108 mmol, 50% in EtOAc) at room temperature under N2 atmosphere. The mixture was stirred at room temperature overnight and quenched with saturated aqueous NH₄Cl. The resulting mixture was extracted with EtOAc twice. The combined organic layers were washed with brine, dried over anhydrous Na₂SO₄, filtered and concentrated to dryness under reduced pressure. The residue was purified by prep-HPLC to give Compound 10 (10 mg, yield 31%) as a white solid. ¹H NMR (400 MHz, CD₃OD) δ 8.47 (s, 1H), 8.20 (d, J=1.3 Hz, 1H), 7.89 (d, J=2.1 Hz, 1H), 7.72 (dd, J=8.6, 2.1 Hz, 1H), 7.45-7.36 (m, 3H), 7.20 (t, J=7.3 Hz, 1H), 7.05 (d, J=8.0 Hz, 2H), 6.81 (d, J=8.6 Hz, 1H), 4.82 (d, J=3.3 Hz, 1H), 4.60-4.50 (m, 2H), 4.48 (s, 2H), 4.34 (q, J=16.6 Hz, 2H), 3.43-3.38 (m, 1H), 2.42 (t, J=12.0 Hz, 1H), 2.19 (d, J=3.3 Hz, 1H), 1.97 (s, 3H), 1.30 (s, 3H), 1.18-1.11 (m, 1H), 0.81 (t, J=6.0 Hz, 1H). LC/MS (ESI) m/z: 603 (M+H)⁺.

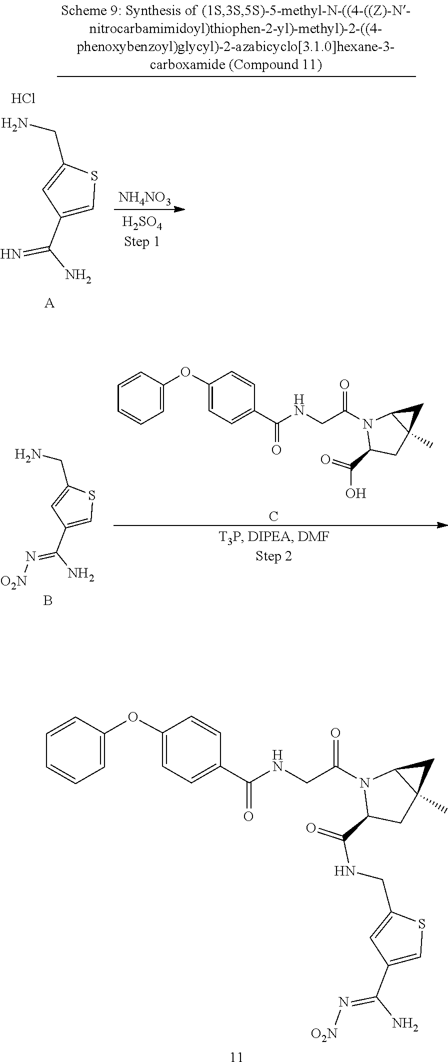

Step 1: (Z)-5-(aminomethyl)-N′-nitrothiophene-3-carboximidamide (B). To a solution of compound A (100 mg, 0.53 mmol) in H₂SO₄ (3 mL) was added NH₄NO₃ (127.2 mg, 1.59 mmol) at 0° C. and the reaction was stirred at room temperature for 0.5 hour. The reaction was quenched with ice-water. The mixture was adjusted to pH˜7 with ammonia and MeOH (20 mL) was added to the mixture. The mixture was filtered and the filtrate was concentrated to dryness under reduced pressure to give compound B (50 mg, yield 47.2%) as a yellow oil, which was used directly in the next step without further purification.

Step 2: (1S,3S,5S)-5-methyl-N-((4-((Z)-N′-nitrocarbamimidoyl)thiophen-2-yl)methyl)-2-((4-phenoxybenzoyl)glycyl)-2-azabicyclo[3.1.0]hexane-3-carboxamide (Compound 11). To a mixture of compound B (50 mg, 0.25 mmol) and compound C (50 mg, 0.127 mmol) in DMF (4 mL) was added DIPEA (81.9 mg, 0.635 mmol) and T₃P (162 mg, 0.254 mmol, 50% in EtOAc) at room temperature under N₂ atmosphere. The mixture was stirred at room temperature overnight and quenched with saturated aqueous NH₄Cl. The resulting mixture was extracted with EtOAc twice. The combined organic layers were washed with brine, dried over anhydrous Na₂SO₄, filtered and concentrated to dryness under reduced pressure. The residue was purified by prep-HPLC to give Compound 11 (8 mg, yield 11%) as a yellow solid. ¹H NMR (400 MHz, CD₃OD) δ 8.51 (s, 1H), 7.86 (d, J=8.7 Hz, 2H), 7.41 (t, J=7.8 Hz, 2H), 7.21 (dd, J=14.9, 7.6 Hz, 2H), 7.07 (d, J=8.0 Hz, 2H), 7.01 (d, J=8.7 Hz, 2H), 4.81 (d, J=3.2 Hz, 1H), 4.55 (s, 2H), 4.32 (s, 2H), 3.42 (d, J=3.8 Hz, 1H), 2.43 (t, J=12.3 Hz, 1H), 2.26-2.08 (m, 1H), 1.29 (t, J=9.3 Hz, 3H), 1.21-1.10 (m, 1H), 0.84 (d, J=5.5 Hz, 1H). LC/MS (ESI) m/z: 577 (M+H)⁺.

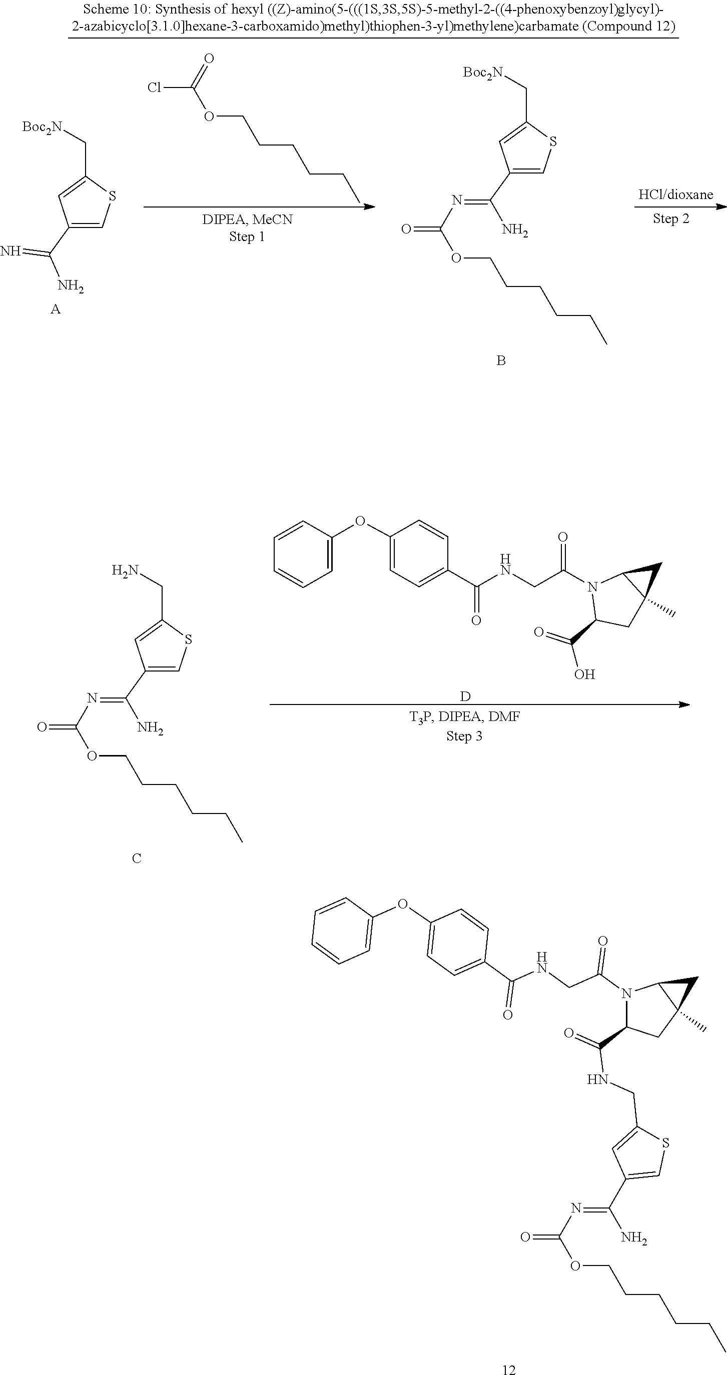

Step 1: hexyl N-[(1Z)-amino(5-{[bis(tert-butoxycarbonyl)amino]methyl}thiophen-3-yl)-methylidene]carbamate (2). To a solution of compound A (50 mg, 0.141 mmol) in MeCN (2 mL) was added DIPEA (0.06 mL, 0.366 mmol) and hexyl chloroformate (23 mg, 0.141 mmol) at 0° C. under N2 atmosphere and the reaction was stirred at 25° C. for 2 hours. The reaction was filtered and concentrated to dryness under reduced pressure. The residue was purified by column chromatography on silica gel (DCM:MeOH=100:1 to 10:1) to give compound B (32 mg, yield 47%) as a yellow oil. LC/MS (ESI) m/z: 384 (M−100+H)⁺.

Step 2: hexyl (Z)-(amino(5-(aminomethyl)thiophen-3-yl)methylene)carbamate (3). A mixture of compound B (32 mg, 0.066 mmol) and HCl/1,4-dioxane (2 mL) was stirred at room temperature for 2 hours and the mixture was concentrated to dryness under reduced pressure to give compound C (18 mg, yield 95%) as a yellow solid, which was used directly in the next step without further purification. LC/MS (ESI) m/z: 284 (M+H)⁺.

Step 3: hexyl ((Z)-amino(5-(((1S,3S,5S)-5-methyl-2-((4-phenoxybenzoyl)glycyl)-2-azabicyclo-[3.1.0]hexane-3-carboxamido)methyl)thiophen-3-yl)methylene)carbamate (Compound 12). To a mixture of compound C (18 mg, 0.064 mmol) and compound D (25 mg, 0.064 mmol) in DMF (2 mL) was added DIPEA (0.06 mL, 0.318 mmol) and T₃P (121 mg, 0.191 mmol, 50% wt. in EtOAc) at 0° C. under N2 atmosphere. The mixture was stirred at 30° C. for 5 hours and quenched with water. The mixture was diluted with EtOAc and separated. The organic layers were washed with brine, dried over anhydrous Na₂SO₄, filtered and concentrated to dryness under reduced pressure. The residue was purified by prep-HPLC to give Compound 12 (1.2 mg, yield 2.86%) as a white solid. ¹H NMR (400 MHz, CD₃OD) δ 8.07 (d, J=1.6 Hz, 1H), 7.83 (d, J=8.8 Hz, 2H), 7.43 (dd, J=25.2, 16.0 Hz, 3H), 7.20 (t, J=7.4 Hz, 1H), 7.07 (d, J=8.0 Hz, 2H), 7.00 (d, J=8.4 Hz, 2H), 4.82 (d, J=3.2 Hz, 1H), 4.57-4.49 (m, 2H), 4.33 (dd, J=47.6, 47.6 Hz, 2H), 4.09 (t, J=7.0 Hz, 2H), 3.38 (dd, J=6.0, 6.0 Hz, 1H), 2.41 (t, J=12.0 Hz, 1H), 2.17 (dd, J=12.8, 13.2 Hz, 1H), 1.70-1.63 (m, 2H), 1.41 (dd, J=13.2, 14.8 Hz, 2H), 1.35-1.30 (m, 4H), 1.29 (s, 3H), 1.17 (dd, J=6.0, 6.0 Hz, 1H), 0.91 (t, J=7.0 Hz, 3H), 0.78 (t, J=5.8 Hz, 1H). LC/MS (ESI) m/z: 660 (M+H)⁺.

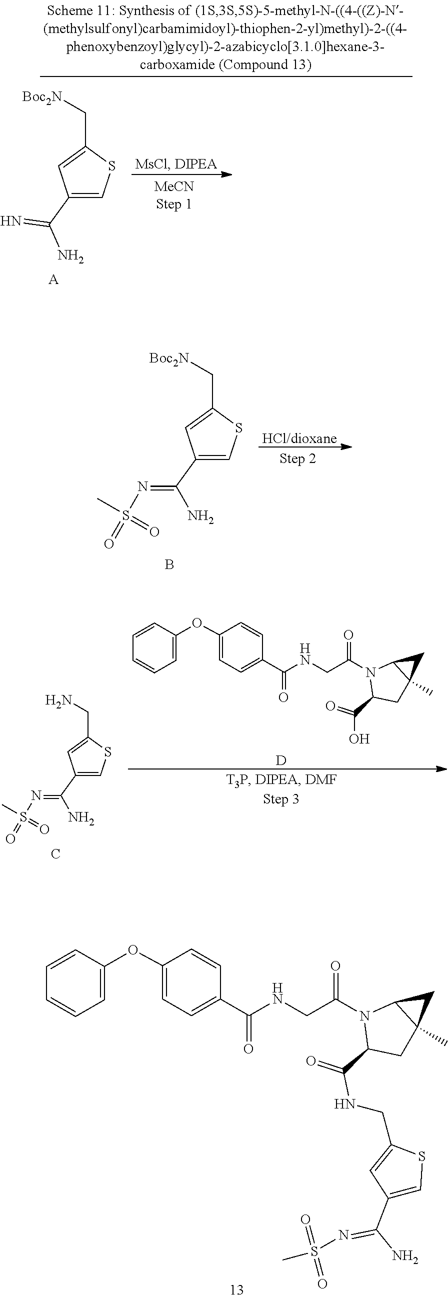

Step 1: tert-butyl N-(tert-butoxycarbonyl)-N-({4-[(Z)-N′-methanesulfonylcarbamimidoyl]-thiophen-2-yl}methyl)carbamate (B). To a solution of compound 1 (50 mg, 0.141 mmol) in MeCN (3 mL) was added DIPEA (0.06 mL, 0.366 mmol) and methanesulfonyl chloride (16 mg, 0.141 mmol) at 0° C. under N2 atmosphere and the mixture was stirred at 25° C. for 2 hours. The mixture was filtered and concentrated to dryness under reduced pressure. The residue was purified by column chromatography on silica gel (DCM:MeOH=100:1 to 10:1) to give compound 2 (47 mg, yield 77.1%) as a yellow oil. LC/MS (ESI) m/z: 334 (M−100+H)⁺.

Step 2: (Z)-5-(aminomethyl)-N′-(methylsulfonyl)thiophene-3-carboximidamide (3). A mixture of compound B (47 mg, 0.108 mmol) and HCl/1,4-dioxane (2 mL) was stirred at room temperature for 3 hours. The reaction was concentrated to dryness under reduced pressure to give compound C (25 mg, yield 98.8%) as yellow solid, which was used directly in the next step without further purification. LC/MS (ESI) m/z: 234 (M+H)⁺.

Step 3: (1S,3S,5S)-5-methyl-N-((4-((Z)-N′-(methylsulfonyl)carbamimidoyl)thiophen-2-yl)-methyl)-2-((4-phenoxybenzoyl)glycyl)-2-azabicyclo[3.1.0]hexane-3-carboxamide (Compound 13). To a mixture of compound C (25 mg, 0.107 mmol) and compound D (42 mg, 0.107 mmol) in DMF (2 mL) was added DIPEA (0.10 mL, 0.536 mmol) and T₃P (205 mg, 0.321 mmol) at 0° C. under N2 atmosphere. The reaction was stirred at 30° C. for 16 hours. The mixture was diluted with EtOAc and the organic layers were washed with brine, dried over anhydrous Na₂SO₄, filtered and concentrated to dryness under reduced pressure. The residue was purified by prep-HPLC to give Compound 13 (3.5 mg, yield 5.4%) as a white solid. ¹H NMR (400 MHz, CD₃OD) δ 8.05 (d, J=1.2 Hz, 1H), 7.83 (d, J=8.8 Hz, 2H), 7.42 (dd, J=13.2, 16 Hz, 3H), 7.19 (t, J=7.6 Hz, 1H), 7.07 (d, J=7.6 Hz, 2H), 7.00 (d, J=8.8 Hz, 2H), 4.81 (d, J=3.2 Hz, 1H), 4.58-4.49 (m, 2H), 4.33 (dd, J=48.4, 48.4 Hz, 2H), 3.40-3.37 (m, 1H), 2.98 (s, 3H), 2.40 (t, J=12.0 Hz, 1H), 2.16 (dd, J=13.2, 13.2 Hz, 1H), 1.29 (s, 3H), 1.17-1.14 (m, 1H), 0.78 (t, J=5.8 Hz, 1H). LC/MS (ESI) m/z: 610 (M+H)⁺.

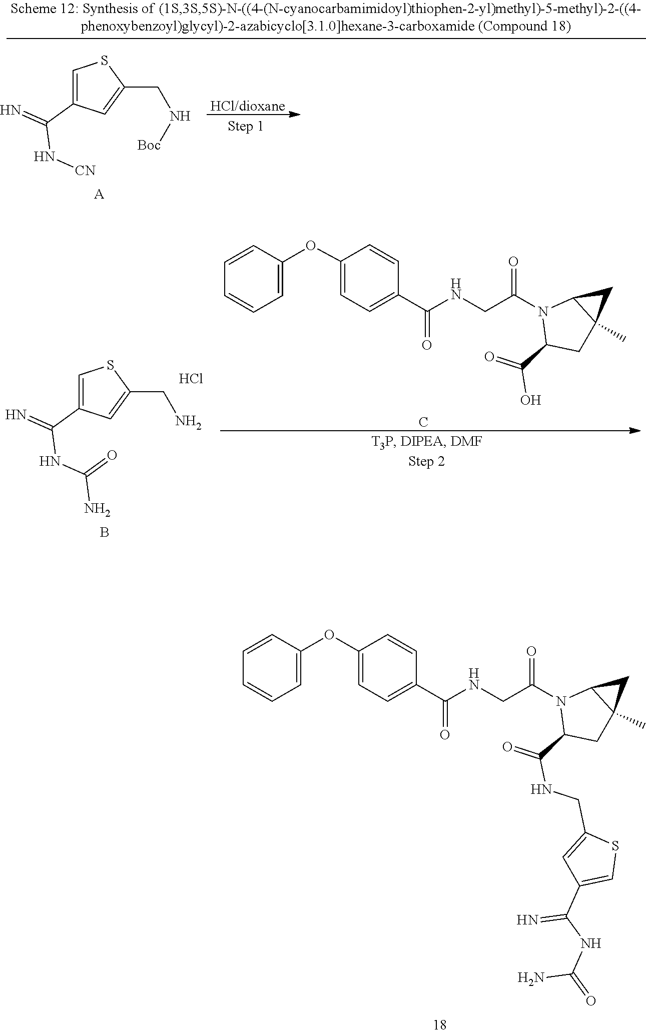

Step 1: 5-(aminomethyl)-N-carbamoylthiophene-3-carboximidamide hydrochloride (B). A solution of compound A (100 mg, 0.36 mmol) in HCl/1,4-dioxane (2 mL) was stirred at room temperature for 3 hours. The mixture was concentrated to dryness under reduced pressure to give compound B (83 mg, 99.3% yield) as a yellow solid, which was used directly in the next step without further purification. LC/MS (ESI) m/z: 199 (M+H)⁺.

Step 2: (1S,3S,5S)-N-((4-(N-cyanocarbamimidoyl)thiophen-2-yl)methyl)-5-methyl-2-((4-phenoxy-benzoyl)glycyl)-2-azabicyclo[3.1.0]hexane-3-carboxamide (Compound 18). To a mixture of compound B (40 mg, 0.10 mmol) and compound C (30 mg, 0.15 mmol) in DMF (2 mL) was added DIPEA (77 mg, 0.6 mmol) and T₃P (191 mg, 0.3 mmol) at 0° C. under N2 atmosphere. The resulting mixture was stirred at room temperature overnight. EtOAc and water were added and the water layer was extracted with EtOAc twice. The combined organic layers were washed with brine, dried over anhydrous Na₂SO₄, filtered and concentrated to dryness under reduced pressure. The residue was purified by prep-HPLC to give Compound 18 (1.9 mg, 3.3% yield) as a white solid. ¹H NMR (400 MHz, CD₃OD) δ 8.23-8.18 (m, 1H), 7.88-7.81 (m, 2H), 7.48-7.37 (m, 3H), 7.24-7.19 (m, 1H), 7.11-7.05 (m, 2H), 7.04-6.98 (m, 2H), 4.84-4.82 (m, 1H), 4.59-4.55 (m, 2H), 4.41-4.26 (m, 2H), 3.43 (dd, J=6.0, 6.0 Hz, 1H), 2.50-2.37 (m, 1H), 2.19 (dd, J=13.2, 13.2 Hz, 1H), 1.31 (s, 3H), 1.17 (dd, J=5.6, 5.6 Hz, 1H), 0.82 (t, J=5.8 Hz, 1H); LC/MS (ESI) m/z: 575 (M+H)⁺.

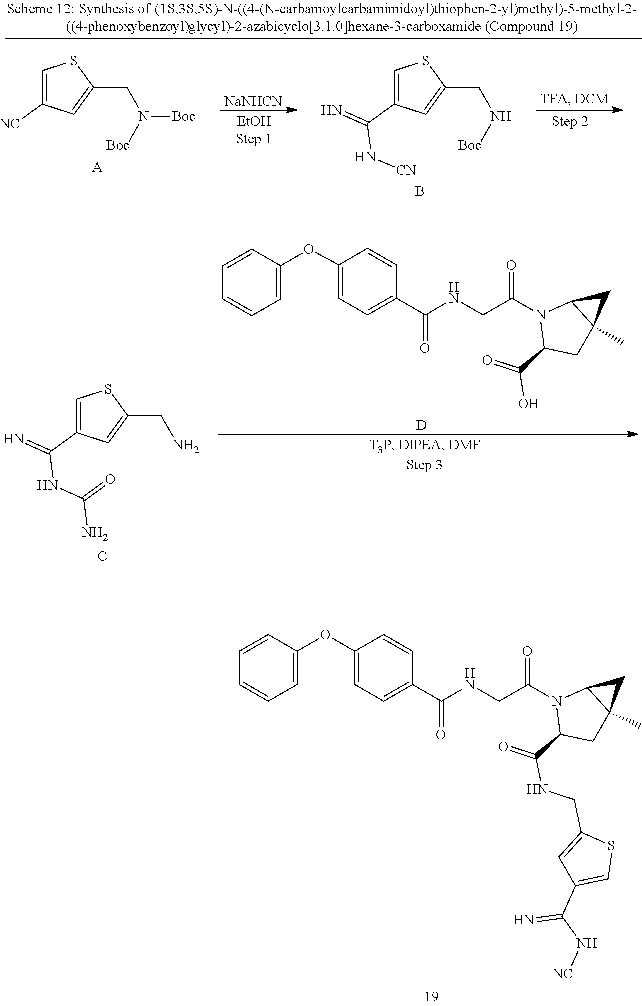

Step 1: tert-butyl ((4-(N-cyanocarbamimidoyl)thiophen-2-yl)methyl)carbamate (B). To a solution of compound A (1 g, 2.96 mmol) in EtOH (10 mL) was added sodium hydrogencyanamide (189 mg, 2.96 mmol) at room temperature under N2 atmosphere and the reaction was stirred at 80° C. overnight. The mixture was concentrated to dryness under reduced pressure and the residue was purified by column chromatography on silica gel (PE:EtOAc=20:1 to 1:1) to give compound B (150 mg, 18.1% yield) as a white solid. LC/MS (ESI) m/z: 281 (M+H)⁺.

Step 2: 5-(aminomethyl)-N-cyanothiophene-3-carboximidamide (C). To a solution of compound B (50 mg, 0.36 mmol) in DCM (2 mL) was added TFA (1 mL) at 0° C. and the reaction was stirred at room temperature for 1 hour. The mixture was concentrated to dryness under reduced pressure to give compound C (32 mg, 99.5% yield) as a yellow solid, which was used directly in the next step. LC/MS (ESI) m/z: 181 (M+H)⁺.

Step 3: (1S,3S,5S)-N-((4-(N-carbamoylcarbamimidoyl)thiophen-2-yl)methyl)-5-methyl-2-((4-phenoxybenzoyl)glycyl)-2-azabicyclo[3.1.0]hexane-3-carboxamide (Compound 19). To a mixture of compound C (30 mg, 0.17 mmol) and compound D (32 mg, 0.08 mmol) in DMF (2 mL) was added DIPEA (62 mg, 0.48 mmol) and T₃P (153 mg, 0.24 mmol) at 0° C. under N2 atmosphere. The resulting mixture was stirred at room temperature overnight. EtOAc and water was added and the mixture was separated. The organic layer was washed with brine, dried over anhydrous Na₂SO₄, filtered and concentrated to dryness under reduced pressure. The residue was purified by prep-HPLC to give Compound 19 (1.2 mg, 2.7% yield) as a white solid. ¹H NMR (400 MHz, CD₃OD) δ 8.13-8.08 (m, 1H), 7.88-7.82 (m, 2H), 7.46-7.40 (m, 3H), 7.24-7.19 (m, 1H), 7.11-7.07 (m, 2H), 7.03-6.99 (m, 2H), 4.85-4.82 (m, 1H), 4.58-4.51 (m, 2H), 4.42-4.27 (m, 2H), 3.40 (dd, J=6.0, 6.0 Hz, 1H), 2.49-2.33 (m, 1H), 2.18 (dd, J=13.2, 13.2 Hz, 1H), 1.30 (s, 3H), 1.15 (dd, J=5.6, 5.6 Hz, 1H), 0.83-0.74 (m, 1H); LC/MS (ESI) m/z: 557 (M+H)⁺.

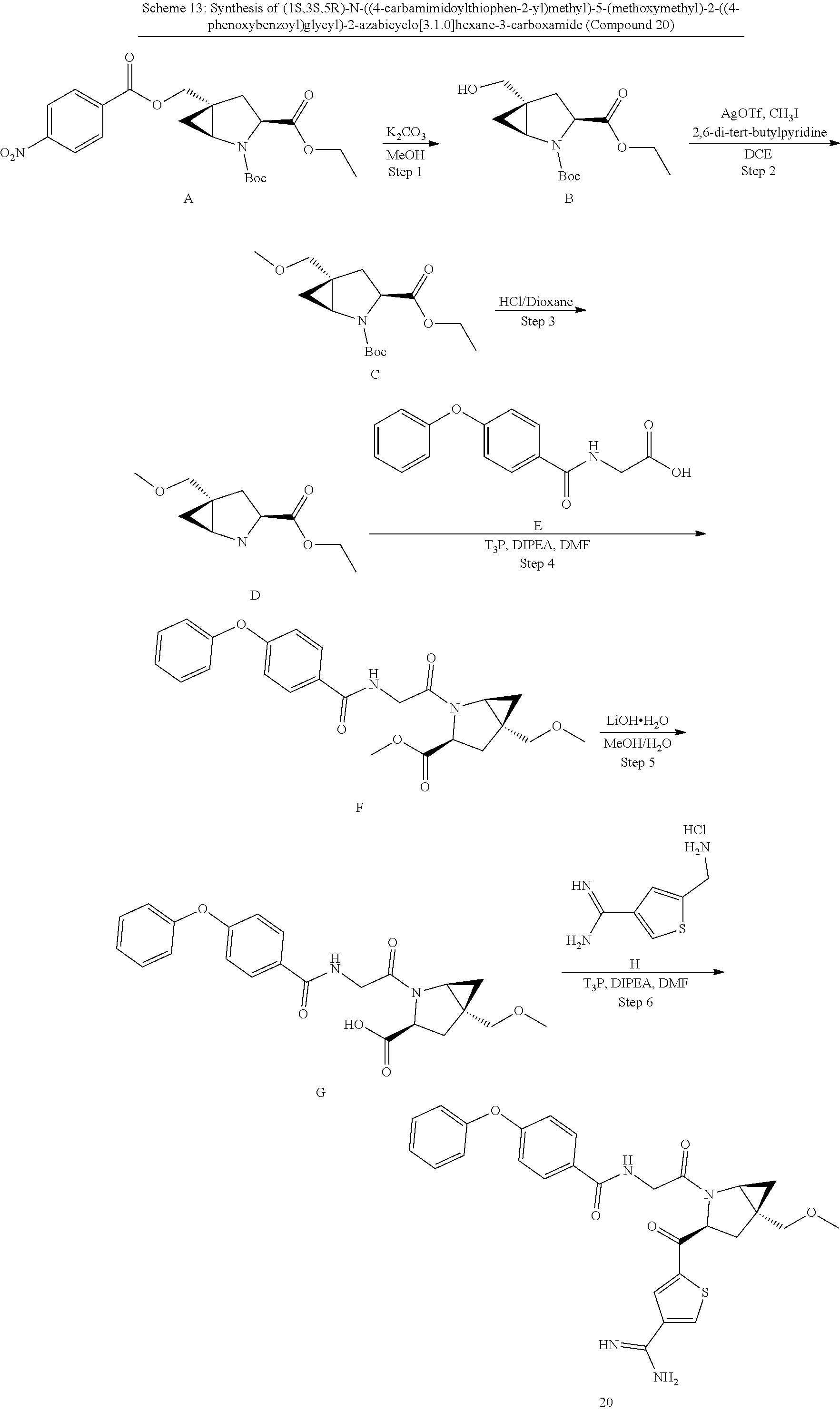

Step 1: 2-(tert-butyl) 3-methyl (1S,3S,5R)-5-(hydroxymethyl)-2-azabicyclo[3.1.0]hexane-2,3-dicarboxylate (B). To a solution of compound A (400 mg, 0.92 mmol) in MeOH (5 mL) was K₂CO₃ (254 mg, 1.84 mmol) at 0° C. under N₂ atmosphere and the mixture was stirred at 25° C. for 16 hours. The reaction was diluted with EtOAc and H₂O and the mixture was separated. The organic layers were washed with brine, dried over anhydrous Na₂SO₄, filtered and concentrated to dryness under reduced pressure. The residue was purified by column chromatography on silica gel (PE:EtOAc=1:1) to give compound B (220 mg, 88% yield) as a colorless oil. LC/MS (ESI) (m/z): 272 (M+H)⁺.

Step 2: 2-(tert-butyl) 3-methyl (1S,3S,5R)-5-(methoxymethyl)-2-azabicyclo[3.1.0]hexane-2,3-dicarboxylate (3). To a solution of compound B (50 mg, 0.18 mmol) in DCE (2 mL) was added AgOTf (71 mg, 0.27 mmol) at 0° C. under N₂ atmosphere, followed by the addition of CH₃I (78 mg, 0.54 mmol) and 2,6-di-tert-butylpyridine (0.12 mL, 0.54 mmol) and the mixture was stirred at 25° C. for 4 hours. The mixture was filtered and concentrated to dryness under reduced pressure. The residue was purified by column chromatography on silica gel (PE:EtOAc=3:1) to give compound C (25 mg, 48.1% yield) as a colorless oil. LC/MS (ESI) (m/z): 286 (M+H)⁺.

Step 3: methyl (1S,3S,5R)-5-(methoxymethyl)-2-azabicyclo[3.1.0]hexane-3-carboxylate (D). A mixture of compound C (25 mg, 0.09 mmol) in HCl/1,4-dioxane (5 mL) was stirred at room temperature for 1 hour. The reaction mixture was concentrated to dryness under reduced pressure to give compound D (16 mg, 93.7% yield) as a yellow oil, which was used directly in the next step. LC/MS (ESI) m/z: 186 (M+H)⁺.

Step 4: methyl (1S,3S,5R)-5-(methoxymethyl)-2-((4-phenoxybenzoyl)glycyl)-2-azabicyclo-[3.1.0]hexane-3-carboxylate (F). To a mixture of compound D (16 mg, 0.09 mmol) and compound E (26 mg, 0.1 mmol) in DMF (3 mL) was added DIPEA (0.09 mL, 0.54 mmol) and T₃P (165 mg, 0.27 mmol) at 0° C. under N2 atmosphere and the mixture was stirred at 25° C. for 16 hours. The mixture was diluted with EtOAc and washed with saturated aq.NH₄Cl solution and brine. The mixture was separated and the organic layer was dried over anhydrous Na₂SO₄, filtered and concentrated to dryness under reduced pressure. The residue was purified by column chromatography on silica gel (PE:EtOAc=1:1) to give compound F (30 mg, 81.1% yield) as light yellow solid. LC/MS (ESI) (m/z): 439 (M+H)⁺.

Step 5: (1S,3S,5R)-5-(methoxymethyl)-2-((4-phenoxybenzoyl)glycyl)-2-azabicyclo[3.1.0]-hexane-3-carboxylic acid (G). To a solution of compound F (30 mg, 0.07 mmol) in MeOH (5 mL) and water (1 mL) was added LiOH·H₂O (6 mg, 0.14 mmol) at 0° C. and the mixture was stirred at room temperature for 16 hours. The mixture was acidified with 1N aq. HCl to pH˜3 and concentrated to dryness under reduced pressure to give compound G (21 mg, 72.4% yield) as a yellow semi-solid, which was used directly in the next step. LC/MS (ESI) (m/z): 425 (M+H)⁺.

Step 6: (1S,3S,5R)-N-((4-carbamimidoylthiophen-2-yl)methyl)-5-(methoxymethyl)-2-((4-phenoxybenzoyl)glycyl)-2-azabicyclo[3.1.0]hexane-3-carboxamide (Compound 20). To a mixture of compound G (25 g, 0.06 mmol) and compound H (14 mg, 0.09 mmol) in DMF (3 mL) was added DIPEA (0.06 mL, 0.36 mmol) and T₃P (0.112 mg, 0.18 mmol) at 0° C. under N2 atmosphere and the mixture was stirred at 25° C. for 16 hours. The mixture was quenched with H₂O and extracted with CHCl₃/i-PrOH (v/v=3/1). The organic layers were dried over anhydrous Na₂SO₄, filtered and concentrated to dryness under reduced pressure. The residue was purified by column chromatography on silica gel (DCM:MeOH=9:1) and further purified by prep-HPLC to give Compound 20 (2.1 mg, 6.4% yield) as a white solid. ¹H NMR (400 MHz, CD₃OD) δ 8.54 (s, 1H), 8.20 (d, J=1.6 Hz, 1H), 7.87-7.80 (m, 2H), 7.45-7.38 (m, 3H), 7.20 (t, J=7.4 Hz, 1H), 7.06 (dd, J=8.6, 1.0 Hz, 2H), 7.03-6.97 (m, 2H), 4.57 (dd, J=14.0, 6.7 Hz, 2H), 4.39-4.28 (m, 2H), 3.59 (dd, J=6.2, 2.7 Hz, 1H), 3.49 (d, J=10.3 Hz, 1H), 3.41 (d, J=10.3 Hz, 1H), 3.36 (s, 3H), 2.64 (t, J=11.8 Hz, 1H), 2.13 (dd, J=13.4, 3.5 Hz, 1H), 1.23 (dd, J=5.9, 2.7 Hz, 1H), 1.00 (t, J=5.6 Hz, 1H); LC/MS (ESI) m/z: 562 (M+H)⁺.

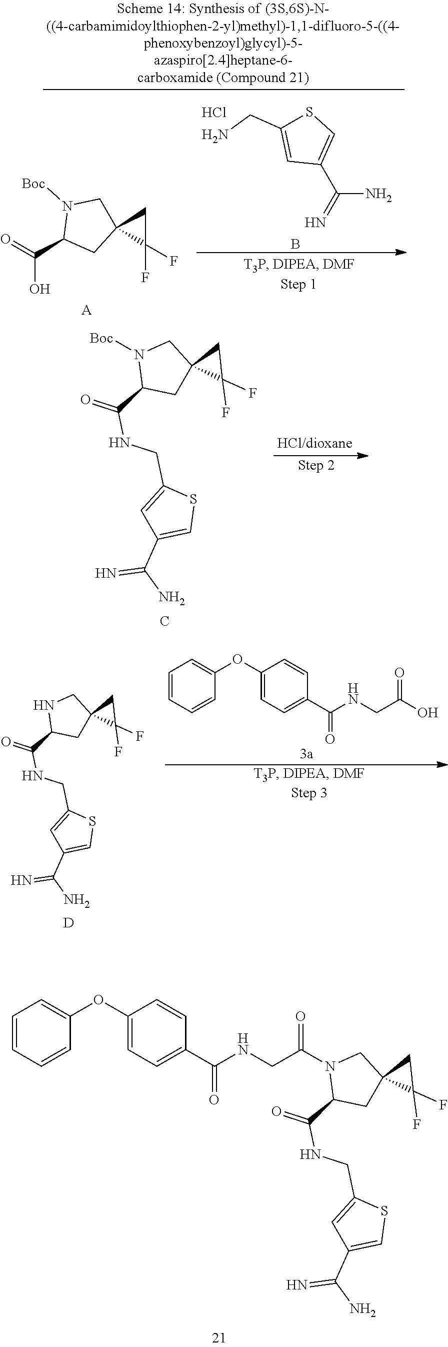

Step 1: tert-butyl (3S,6S)-6-(((4-carbamimidoylthiophen-2-yl)methyl)carbamoyl)-1,1-difluoro-5-azaspiro[2.4]heptane-5-carboxylate (C). To a mixture of compound A (50 mg, 0.18 mmol) and compound B (42 mg, 0.27 mmol) in DMF (5 mL) was added DIPEA (0.18 mL, 1.08 mmol) and T₃P (344 mg, 0.54 mmol) at 0° C. under N₂ atmosphere and the mixture was stirred at room temperature for 16 hours. The mixture was quenched with H₂O and extracted with CHCl₃/i-PrOH (v/v=3/1) twice. The organic layers were dried over anhydrous Na₂SO₄, filtered and concentrated to dryness under reduced pressure. The residue was purified by column chromatography on silica gel (DCM:MeOH=4:1) to give compound C (70 mg, 94.6% yield) as white solid. LC/MS (ESI) (m/z): 415 (M+H)⁺.

Step 2: (3S,6S)-N-((4-carbamimidoylthiophen-2-yl)methyl)-1,1-difluoro-5-azaspiro[2.4]-heptane-6-carboxamide (2). A mixture of compound 2 (70 mg, 0.17 mmol) in HCl/1,4-dioxane (5 mL) was stirred at 0° C. and the mixture was stirred at room temperature for 2 hours. The mixture was concentrated to dryness under reduced pressure to give compound 3 (50 mg, 94.3% yield) as white solid, which was used directly in next step. LC/MS (ESI) (m/z): 315 (M+H)⁺.

Step 3: (3S,6S)-N-((4-carbamimidoylthiophen-2-yl)methyl)-1,1-difluoro-5-((4-phenoxy-benzoyl)glycyl)-5-azaspiro[2.4]heptane-6-carboxamide (Compound 21). To a mixture of compound 3 (56 mg, 0.18 mmol) and compound 3a (53 mg, 0.20 mmol) in DMF (5 mL) was added DIPEA (0.18 mL, 1.08 mmol) and T₃P (340 mg, 0.54 mmol) at 0° C. under N2 atmosphere and the mixture was stirred at room temperature for 16 hours. The mixture was quenched with H₂O and extracted with CHCl₃/i-PrOH (v/v=3/1) twice. The organic layers were dried over anhydrous Na₂SO₄, filtered and concentrated to dryness under reduced pressure. The residue was purified by column chromatography on silica gel (DCM:MeOH=4:1) and further purified by prep-HPLC to give Compound 21 (1.8 mg, 1.8% yield) as white solid. ¹H NMR (400 MHz, CD₃OD) δ 8.49 (s, 1H), 8.21 (d, J=1.6 Hz, 1H), 7.87-7.82 (m, 2H), 7.42 (dd, J=10.8, 5.2 Hz, 3H), 7.20 (t, J=7.4 Hz, 1H), 7.08-7.04 (m, 2H), 7.03-6.98 (m, 2H), 4.66 (dd, J=8.7, 4.2 Hz, 1H), 4.59 (s, 2H), 4.16 (dt, J=16.6, 15.1 Hz, 2H), 4.03-3.93 (m, 1H), 3.78 (dd, J=10.6, 4.2 Hz, 1H), 2.59 (dd, J=12.5, 9.0 Hz, 1H), 2.04 (dt, J=15.0, 4.6 Hz, 1H), 1.58 (ddd, J=13.2, 8.3, 4.6 Hz, 1H), 1.52-1.42 (m, 1H); LC/MS (ESI) m/z: 568(M+H)⁺.

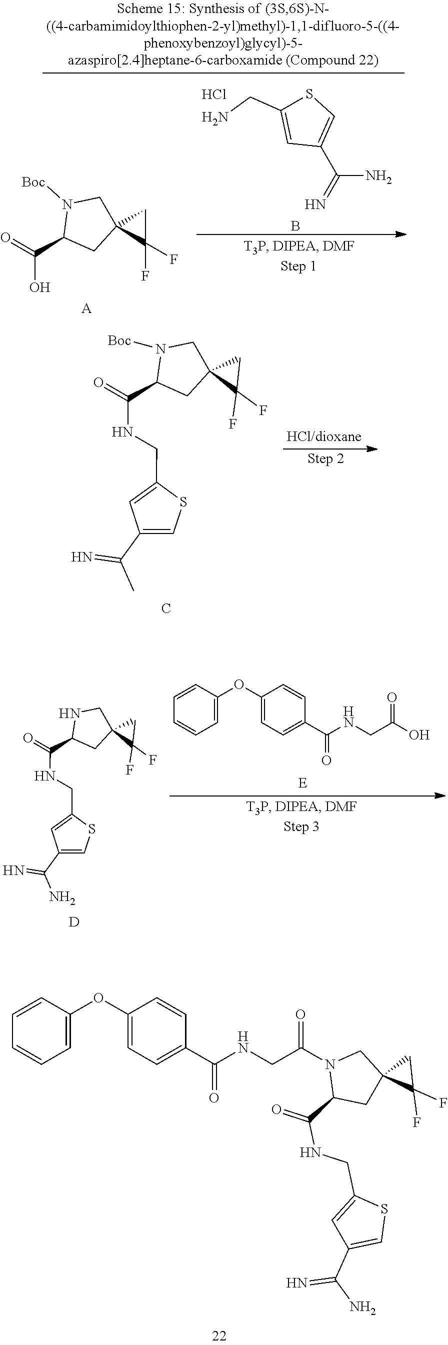

Step 1: tert-butyl (3R,6S)-1,1-difluoro-6-(((4-(1-iminoethyl)thiophen-2-yl)methyl)carbamoyl)-5-azaspiro[2.4]heptane-5-carboxylate (C). To a mixture compound A (50 mg, 0.18 mmol) and compound B (34.5 mg, 0.18 mmol) in DMF (1 mL) was added DIPEA (0.18 mL, 1.08 mmol) and T₃P (344 mg, 0.54 mmol) at 0° C. under N₂ atmosphere. The mixture was stirred at 30° C. for 3 hours. EtOAc and water were added and the mixture was separated. The water layer was extracted with EtOAc twice and the combined organic layers were dried over anhydrous Na₂SO₄, filtered and concentrated to dryness under reduced pressure. The residue was purified by column chromatography on silica gel (DCM:MeOH=100:1 to 5:1) to give compound C (74 mg, 99.0% yield) as a white solid. LC/MS (ESI) m/z: 358 (M−56+H)⁺.

Step 2: (3R,6S)-N-((4-carbamimidoylthiophen-2-yl)methyl)-1,1-difluoro-5-azaspiro-[2.4]heptane-6-carboxamide (D). A mixture of compound C (74 mg, 0.18 mmol) in HCl/1,4-dioxane (2 mL) was stirred at room temperature for 2 hours. The reaction mixture was concentrated to dryness under reduced pressure to give compound D (56 mg, 99.0% yield) as a white solid, which was used directly in the next step. LC/MS (ESI) m/z: 315 (M+H)⁺.

Step 3: (3R,6S)-N-((4-carbamimidoylthiophen-2-yl)methyl)-1,1-difluoro-5-((4-phenoxy-benzoyl)glycyl)-5-azaspiro[2.4]heptane-6-carboxamide (Compound 22). To a mixture of compound D (56 mg, 0.18 mmol) and compound E (49 mg, 0.18 mmol) in DMF (1 mL) was added DIPEA (0.18 mL, 1.08 mmol) and T₃P (344 mg, 0.54 mmol) at 0° C. under N2 atmosphere. The mixture was stirred at 30° C. for 16 hours and quenched with water. The mixture was extracted with EtOAc and the combined organic layers were dried over anhydrous Na₂SO₄, filtered and concentrated to dryness under reduced pressure. The residue was purified by prep-HPLC to give Compound 22 (2.0 mg, 2.0% yield) as a white solid. ¹H NMR (400 MHz, CD₃OD) δ 8.51 (s, 1H), 8.22 (dd, J=18.0, 17.2 Hz, 1H), 7.87-7.79 (m, 2H), 7.44 (dd, J=36.0, 16.0 Hz, 3H), 7.20 (t, J=7.6 Hz, 1H), 7.09-7.04 (m, 2H), 7.01 (dd, J=8.4, 8.8 Hz, 2H), 4.73 (dd, J=28.8, 28.0 Hz, 1H), 4.64-4.51 (m, 2H), 4.18 (dd, J=44.0, 48.0 Hz, 2H), 4.00-3.82 (m, 2H), 2.56-2.47 (m, 1H), 2.17 (dd, J=32.0, 31.2 Hz, 1H), 1.64-1.51 (m, 2H); LC/MS (ESI) m/z: 568 (M+H)⁺.

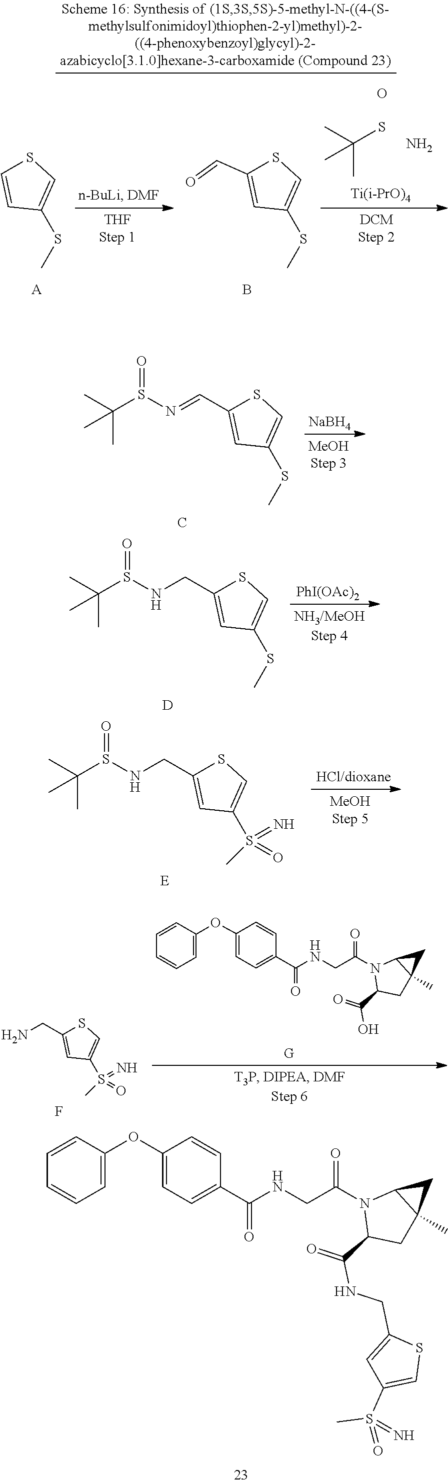

Step 1: 4-(methylthio)thiophene-2-carbaldehyde (B). To a solution of n-BuLi (21.1 mL, 33.7 mmol) in THE (40 mL) was added compound A (4 g, 30.7 mmol) in portions at −40° C. under N₂ atmosphere. The mixture was stirred at 0° C. for 10 minutes and further 10 minutes at room temperature. The mixture was cooled to −70° C. again and DMF (2.82 g, 36.8 mmol) was added. The reaction was stirred at −70° C. for 30 minutes and quenched with sat. NH₄Cl. The mixture was extracted with EtOAc twice and the combined organic layers were washed with brine, dried over anhydrous Na₂SO₄ and concentrated to dryness under reduced pressure. The residue was purified by column chromatography on silica gel (PE:EtOAc=1:0 to 10:1) to give compound B (1.6 g, 32.9% yield) as a yellow oil. LC/MS (ESI) m/z: 159 (M+H)⁺.

Step 2: (E)-2-methyl-N-((4-(methylthio)thiophen-2-yl)methylene)propane-2-sulfinamide (C). A mixture of compound B (1.3 g, 8.2 mmol), Ti(i-PrO)₄ (7.0 g, 24.6 mmol) and tert-butanesulfinamide (1.5 g, 12.3 mmol) in DCM (13 mL) was stirred at room temperature for 4 hours and the reaction was quenched with H₂O. The mixture was filtered through a plug of celite and the filtrate was partitioned between EtOAc and water. The layers were separated and the organic layer was washed with brine, dried over Na₂SO₄, filtered and concentrated to dryness under reduced pressure to give compound C (1.8 g, 83.8% yield), which was used directly in the next step. LC/MS (ESI): m/z 262 (M+H)⁺.

Step 3: N-((4-(methylthio)thiophen-2-yl)methyl)pivalamide (D). To a solution of compound C (1.8 g, 6.8 mmol) in MeOH (18 mL) was added NaBH₄ (0.52 g, 13.7 mmol) in portions at 0° C. under N2 atmosphere. The reaction was stirred at room temperature for 0.5 hour and quenched with saturated NH₄Cl solution. The mixture was extracted with EtOAc twice. The combined organic layers were washed with brine, dried over anhydrous Na₂SO₄, filtered and concentrated to dryness under reduced pressure to give compound D (1.6 g, 88.9% yield), which was used directly in the next step. LC/MS (ESI) m/z: 264 (M+H)⁺.

Step 4: N-((4-(S-methylsulfonimidoyl)thiophen-2-yl)methyl)pivalamide (E). To a solution of compound D (1.6 g, 6.0 mmol) in MeOH (30 mL) was added Ph(OAc)₂ (4.44 g, 13.8 mmol) and NH₃/MeOH (2.25 mL, 9.0 mmol, 4M) at 0° C. under N2 atmosphere and the mixture was stirred at room temperature for 0.5 hour. The reaction was concentrated to dryness under reduced pressure and the residue was purified by column chromatography on silica gel (DCM:MeOH=1:0 to 10:1) to give compound E (1.0 g, 56.1% yield) as a white solid. LC/MS (ESI) m/z: 295 (M+H)⁺.

Step 5: (5-(aminomethyl)thiophen-3-yl)(imino)(methyl)-16-sulfanone (F). A mixture of compound E (0.4 g, 1.36 mmol) and HCl/1,4-dioxane (6 mL) was stirred at room temperature for 1 hour and the mixture was concentrated to dryness under reduced pressure to give compound F (0.21 g, 84.0% yield) as a white solid, which was used directly in the next step. LC/MS (ESI) m/z: 191 (M+H)⁺.

Step 6: (1S,3S,5S)-5-methyl-N-((4-(S-methylsulfonimidoyl)thiophen-2-yl)methyl)-2-((4-phenoxybenzoyl)glycyl)-2-azabicyclo[3.1.0]hexane-3-carboxamide (Compound 23). To a mixture of compound F (28.5 mg, 0.15 mmol) and G (25 mg, 0.075 mmol) in DMF (5 mL) was added DIPEA (50.0 mg, 0.375 mmol) and T₃P (145 mg, 0.225 mmol) at room temperature under N2 atmosphere and the mixture was stirred at 30° C. for 16 hours. The mixture was diluted with EtOAc and washed with saturated aq.NH₄Cl solution and brine. The organic layer was dried over anhydrous Na₂SO₄, filtered and concentrated to dryness under reduced pressure. The residue was purified by prep-HPLC to give Compound 23 (5.0 mg, 12.0% yield) as a white solid. ¹H NMR (400 MHz, CD₃OD) δ 8.02 (d, J=1.5 Hz, 1H), 7.85 (d, J=8.4 Hz, 2H), 7.41 (dd, J=8.5, 7.5 Hz, 2H), 7.34 (s, 1H), 7.21 (d, J=7.4 Hz, 1H), 7.07 (dd, J=8.6, 1.0 Hz, 2H), 7.02-6.99 (m, 2H), 4.80 (d, J=3.4 Hz, 1H), 4.54 (dd, J=19.4, 3.5 Hz, 2H), 4.40 (dd, J=16.5, 3.5 Hz, 1H), 4.27 (dd, J=16.6, 2.6 Hz, 1H), 3.39 (dd, J=6.0, 2.4 Hz, 1H), 3.11 (d, J=14.0 Hz, 3H), 2.40 (t, J=12.4 Hz, 1H), 2.19-2.12 (m, 1H), 1.29 (s, 3H), 1.16 (t, J=6.2 Hz, 1H), 0.79 (t, J=5.9 Hz, 1H); LC/MS (ESI) m/z: 567 (M+H)⁺.

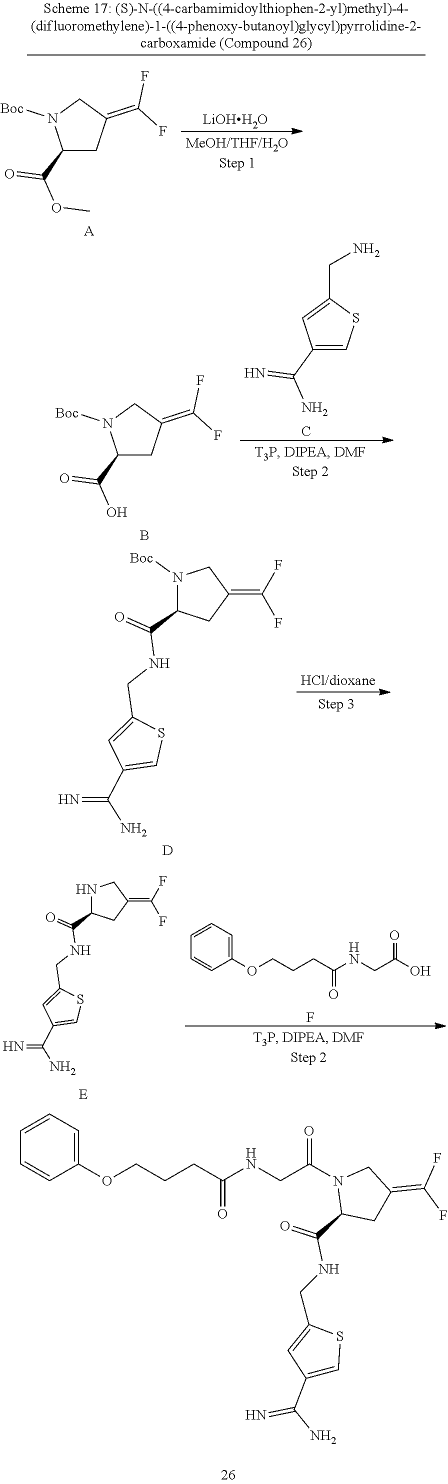

Step 1: (S)-1-(tert-butoxycarbonyl)-4-(difluoromethylene)pyrrolidine-2-carboxylic acid (B). To a solution of compound A (100 mg, 0.36 mmol) in MeOH (1 mL) and THE (0.5 mL) was added a solution of LiOH·H₂O (15 mg, 0.36 mmol) in H₂O (0.5 mL) at 0° C. and the mixture was stirred at 25° C. for 4 hours. The mixture was diluted with water and extracted with EtOAc twice. The water layer were acidified with 0.5 M HCl and extracted with EtOAc twice. The organic layers were washed with brine, dried over anhydrous Na₂SO₄, filtered and concentrated to dryness under reduced pressure to give compound B (94 mg, 99% yield) as a yellow oil, which was used directly in the next step. LC/MS (ESI) m/z: 208 (M−56+H)⁺.

Step 2: tert-butyl (S)-2-(((4-carbamimidoylthiophen-2-yl)methyl)carbamoyl)-4-(difluoro-methylene)pyrrolidine-1-carboxylate (D). To a mixture of compound B (94 mg, 0.36 mmol) and compound C (111 mg, 0.71 mmol) in DMF (1 mL) was added DIPEA (0.37 mL, 2.14 mmol) and T₃P (682 mg, 1.07 mmol) at 0° C. under N₂ atmosphere and the mixture was stirred at 30° C. for 5 hours. The mixture was diluted with water and extracted with CH₃Cl/i-PrOH (V/V=3/1). The combined organic layers were washed with brine, dried over anhydrous Na₂SO₄, filtered and concentrated to dryness under reduced pressure. The residue was purified by column chromatography on silica gel (DCM:MeOH=100:1 to 5:1) to give compound D (60 mg, 42% yield) as a yellow oil. LC/MS (ESI) m/z: 301 (M−100+H)⁺.

Step 3: (S)-N-((4-carbamimidoylthiophen-2-yl)methyl)-4-(difluoromethylene)pyrrolidine-2-carboxamide (E). A mixture of compound D (60 mg, 0.15 mmol) in HCl/1,4-dioxane (2 mL) was stirred at room temperature for 2 hours. The reaction was concentrated to dryness under reduced pressure to give compound E (45 mg, 100% yield) as a white solid, which was used directly in the next step. LC/MS (ESI) m/z: 301 (M+H)⁺.

Step 4: (S)-N-((4-carbamimidoylthiophen-2-yl)methyl)-4-(difluoromethylene)-1-((4-phenoxy-butanoyl)glycyl)pyrrolidine-2-carboxamide (Compound 26). To a mixture of compound E (45 mg, 0.15 mmol) and compound F (36 mg, 0.15 mmol) in DMF (1.5 mL) was added DIPEA (0.16 mL, 0.9 mmol) and T₃P (286 mg, 0.45 mmol) at 0° C. under N2 atmosphere and the mixture was stirred at 30° C. for 16 hours. The mixture was diluted with water and extracted with CH₃Cl/i-PrOH (V/V=3/1) twice. The combined organic layers were washed with saturated NaHCO₃ solution and brine, dried over anhydrous Na₂SO₄, filtered, and concentrated to dryness under reduced pressure. The residue was purified by prep-HPLC to give Compound 26 (3 mg, 4% yield) as a white solid. ¹H NMR (400 MHz, CD₃OD) δ 8.55 (s, 1H), 8.22 (t, J=2.6 Hz, 1H), 7.43 (d, J=26.4 Hz, 1H), 7.23 (t, J=8.0 Hz, 2H), 6.92-6.87 (m, 3H), 4.75-4.64 (m, 1H), 4.63-4.50 (m, 2H), 4.39-4.14 (m, 2H), 4.08-3.92 (m, 4H), 3.14-2.88 (m, 1H), 2.74 (dd, J=45.6, 46.0 Hz, 1H), 2.47 (t, J=7.4 Hz, 2H), 2.11-2.03 (m, 2H); LC/MS (ESI) m/z: 520 (M+H)⁺.

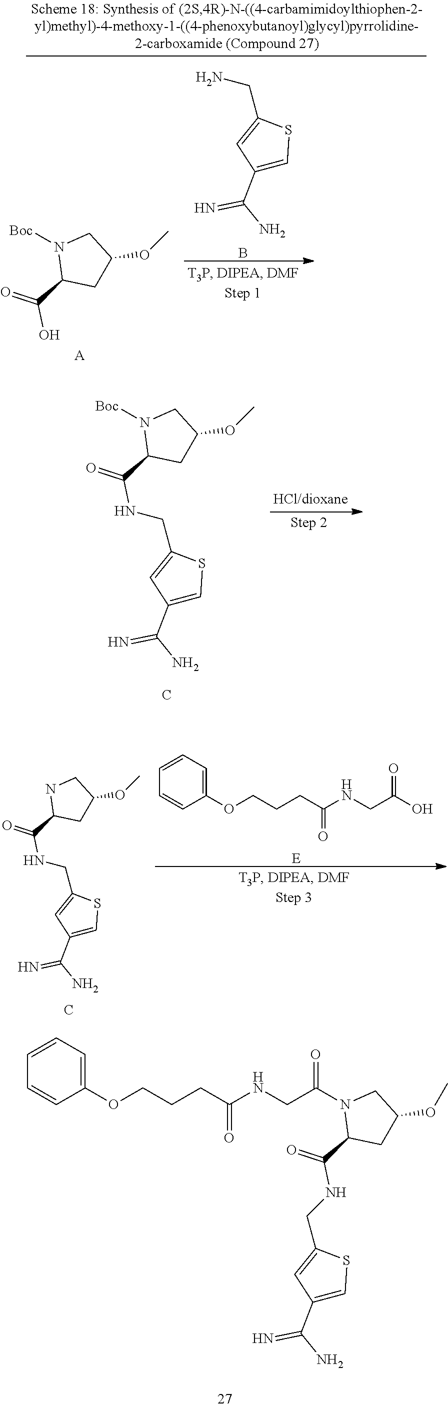

Step 1: tert-butyl (2S,4R)-2-(((4-carbamimidoylthiophen-2-yl)methyl)carbamoyl)-4-methoxypyrrolidine-1-carboxylate (C). To a mixture of compound A (100 mg, 0.41 mmol) and compound B (126 mg, 0.82 mmol) in DMF (3 mL) was added DIPEA (0.41 mL, 2.46 mmol) and T₃P (391 mg, 1.23 mmol) at 0° C. under N2 atmosphere and the mixture was stirred at room temperature for 16 hours. The mixture was diluted with EtOAc and washed with saturated aq. NH₄Cl solution and brine. The organic layer was separated and dried over anhydrous Na₂SO₄, filtered and concentrated to dryness under reduced pressure. The residue was purified by column chromatography on silica gel (DCM:MeOH=10:1) to give compound C (110 mg, 70.5% yield) as a colorless oil. LC/MS (ESI) (m/z): 383 (M+H)⁺.

Step 2: (2S,4R)-N-((4-carbamimidoylthiophen-2-yl)methyl)-4-methoxypyrrolidine-2-carboxamide hydrochloride (D). A mixture of compound C (110 mg, 0.29 mmol) and HCl/1,4-dioxane (2 mL) was stirred at room temperature for 3 hours. The reaction was concentrated to dryness under reduced pressure to give compound D (92 mg, 99.8% yield) as a yellow solid, which was used directly in the next step. LC/MS (ESI) m/z: 283 (M+H)⁺.

Step 3: (2S,4R)-N-((4-carbamimidoylthiophen-2-yl)methyl)-4-methoxy-1-((4-phenoxy-butanoyl)glycyl)pyrrolidine-2-carboxamide (Compound 27). To a mixture of compound D (40 mg, 0.13 mmol) and compound E (30 mg, 0.13 mmol) in DMF (3 mL) was added DIPEA (0.13 mL, 0.78 mmol) and T₃P (124 mg, 0.39 mmol) at 0° C. under N2 atmosphere. The reaction was stirred at room temperature for 16 hours. The mixture was diluted with CH₃Cl/i-PrOH (V/V=3/1) and the organic layers were washed with saturated NaHCO₃ solution and brine, dried over anhydrous Na₂SO₄, filtered and concentrated to dryness under reduced pressure. The residue was purified by prep-HPLC to give Compound 27 (2.2 mg, 3.5% yield) as a white solid. ¹H NMR (400 MHz, CD₃OD) δ 8.54 (s, 1H), 8.24-8.22 (m, 1H), 7.45 (s, 1H), 7.25 (t, J=8.0 Hz, 2H), 6.93-6.89 (m, 3H), 4.60-4.53 (m, 2H), 4.45 (t, J=8.0 Hz, 1H), 4.12-4.08 (m, 1H), 4.05-3.99 (m, 4H), 3.74 (d, J=3.2 Hz, 2H), 3.36 (s, 3H), 2.50-2.46 (m, 2H), 2.41-2.33 (m, 1H), 2.11-2.01 (m, 3H); LC/MS (ESI) m/z: 502 (M+H)⁺.

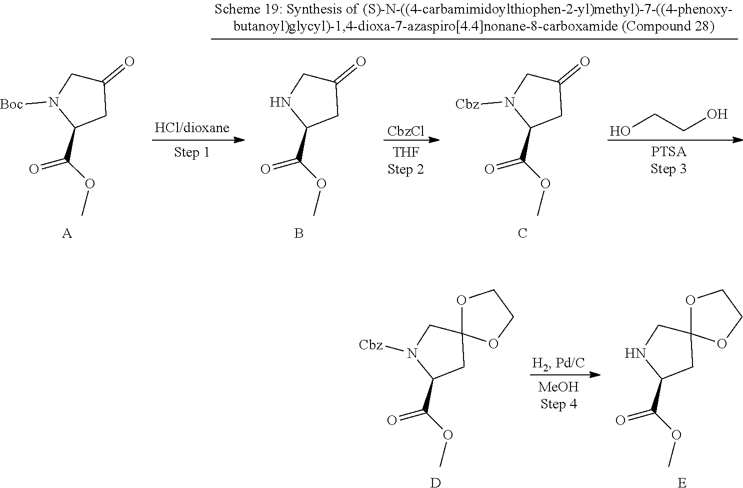

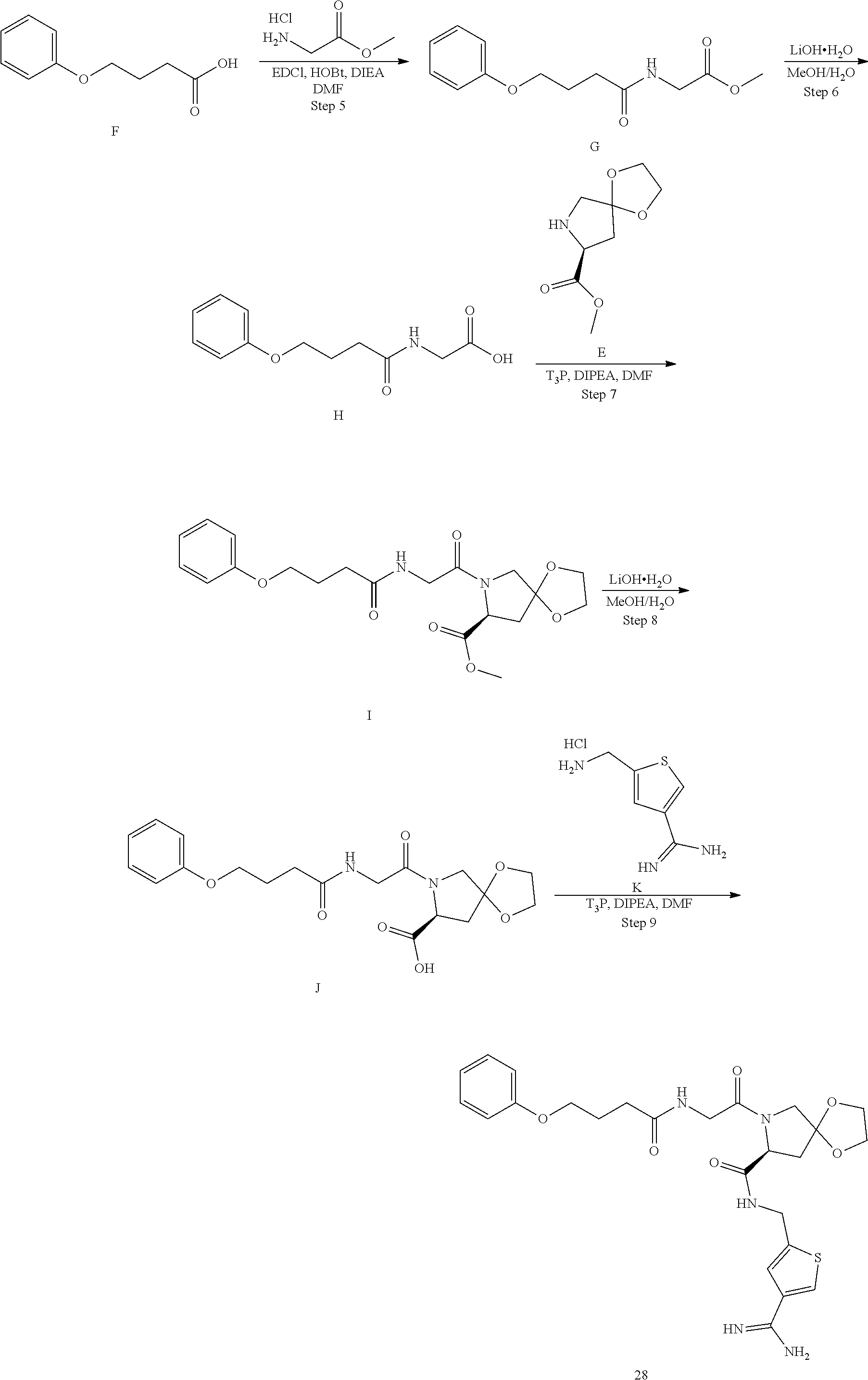

Step 1: methyl (S)-4-oxopyrrolidine-2-carboxylate (B). A mixture of compound A (500 mg, 2.06 mmol) and HCl/1,4-dioxane (5 mL) was stirred at room temperature for 1 hour. The reaction mixture was concentrated to dryness under reduced pressure to give compound 2 (294 mg, 100% yield) as a yellow solid, which was used directly in the next step. LC/MS (ESI) m/z: 144(M+H)⁺.

Step 2: 1-benzyl 2-methyl (S)-4-oxopyrrolidine-1,2-dicarboxylate (C). To a mixture of compound B (294 mg, 2.06 mmol) and NaHCO₃ (344.4 mg, 4.12 mmol) in THE (5 mL) and H₂O (5 mL) was added benzyl chloroformate (455 mg, 2.68 mmol) dropwise at 0° C. under N₂ atmosphere. The reaction was stirred at room temperature overnight. The mixture was diluted with water and extracted with EtOAc twice. The combined organic layers were washed with brine, dried over anhydrous Na₂SO₄, filtered and concentrated to dryness under reduced pressure. The residue was purified by column chromatography on silica gel (PE:EtOAc=10:1 to 3:1) to give compound C (410 mg, 72% yield) as a colorless oil.

Step 3: 7-benzyl 8-methyl (S)-1,4-dioxa-7-azaspiro[4.4]nonane-7,8-dicarboxylate (D). To a solution of compound C (410 mg, 1.48 mmol) in toluene (8 mL) was added ethylene glycol (367 mg, 5.92 mmol) and PTSA (141 mg, 0.74 mmol) at room temperature under N₂ atmosphere. The reaction was stirred at 140° C. for 6 hours. The mixture was cooled down to room temperature, diluted with water and extracted with EtOAc twice. The combined organic layers were washed with brine, dried over anhydrous Na₂SO₄, filtered and concentrated to dryness under reduced pressure. The residue was purified by column chromatography on silica gel (PE:EtOAc=10:1 to 3:2) to give compound D (255 mg, 54% yield) as a light-yellow oil. LC/MS (ESI) m/z: 322 (M+H)⁺.

Step 4: methyl (S)-1,4-dioxa-7-azaspiro[4.4]nonane-8-carboxylate (E). To a stirred solution of compound D (255 mg, 0.795 mmol) in MeOH was added 10% Pd/C (30 mg) under N2 atmosphere and the reaction was stirred at room temperature under H₂ atmosphere for 2 hours. The mixture was filtered and concentrated to dryness under reduced pressure to give compound E (130 mg, 88% yield) as colorless oil, which was used directly in the next step. LC/MS (ESI) m/z: 188 (M+H)⁺.

Step 5: methyl (4-phenoxybutanoyl)glycinate (G). To a solution of compound F (15.0 g, 83.3 mmol) in DMF (150 mL) was added DIPEA (43.0 g, 333.2 mmol), methyl glycinate hydrochloride (15.7 g, 124.9 mmol), HOBt (15.7 g, 116.6 mmol) and EDCI (20.7 g, 108.3 mmol) at room temperature under N2 atmosphere and the mixture was stirred at room temperature overnight. The reaction was quenched with saturated aq.NH₄Cl solution and extracted with EtOAc twice. The combined organic layers were washed with brine, dried over Na₂SO₄, filtered, and concentrated to dryness under reduced pressure. The residue was purified by column chromatography on silica gel (PE:EtOAc=:1 to 2:1) to give compound G (20.1 g, 96% yield) as a light-yellow oil. LC/MS (ESI) m/z: 252 (M+H)⁺.

Step 6: (4-phenoxybutanoyl)glycine (H). To a solution of compound G (10.3 g, 41 mmol) in MeOH (30 mL) and water (10 mL) was added LiOH·H₂O (3.5 g, 82 mmol) at 0° C. and the mixture was stirred at room temperature for 16 hours. The mixture was acidified with 1N aq. HCl to pH˜3 and filtered. The residue was washed with water and dried under reduced pressure to give compound H (6.6 g, 68% yield) as a white solid, which was used directly in the next step. LC/MS (ESI) (m/z): 238 (M+H)⁺.

Step 7: (4-phenoxybutanoyl)glycine (1). To a mixture of compound H (110 mg, 0.464 mmol) and compound E (130.1 mg, 0.696 mmol) in DMF (3 mL) was added DIPEA (239 mg, 1.86 mmol) and T₃P (590 mg, 0.928 mmol, 50% in EtOAc) at room temperature under N2 atmosphere and the mixture was stirred at room temperature overnight. The reaction was quenched with saturated aqueous NH₄Cl solution and extracted with EtOAc twice. The combined organic layers were washed with brine, dried over Na₂SO₄, filtered and concentrated to dryness under reduced pressure. The residue was purified by column chromatography on silica gel (PE:EtOAc=5:1 to 1:1) to give compound I (62 mg, 33% yield) as a light-yellow oil. LC/MS (ESI) m/z: 407 (M+H)⁺.

Step 8 (S)-7-((4-phenoxybutanoyl)glycyl)-1,4-dioxa-7-azaspiro[4.4]nonane-8-carboxylic acid (J). To a solution of compound I (62 mg, 0.153 mmol) in MeOH (1.5 mL) and water (0.5 mL) was added LiOH·H₂O (6.6 mg, 0.153 mmol) at 0° C. and the mixture was stirred at room temperature for 16 hours. The mixture was acidified with 1 N aq. HCl to pH˜3 and extracted with EtOAc twice. The organic layer was dried over anhydrous Na₂SO₄, filtered, and concentrated to dryness under reduced pressure to give compound J (50 mg, 84% yield) as a white solid, which was used directly in next step. LC/MS (ESI) (m/z): 393 (M+H)⁺.

Step 9: (S)-N-((4-carbamimidoylthiophen-2-yl)methyl)-7-((4-phenoxybutanoyl)glycyl)-1,4-dioxa-7-azaspiro[4.4]nonane-8-carboxamide (Compound 28). To a mixture of compound J (50 mg, 0.128 mmol) and compound K (36.3 mg, 0.191 mmol) in DMF (3 mL) was added DIPEA (49.5 mg, 0.384 mmol) and T₃P (163 mg, 0.256 mmol, 50% in EtOAc) at room temperature under N₂ atmosphere. The mixture was stirred at room temperature overnight and quenched with saturated aqueous NH₄Cl. The resulting mixture was extracted with CH₃Cl/i-PrOH (V/V=3/1) twice. The combined organic layers were washed with saturated NaHCO₃ solution and brine, dried over anhydrous Na₂SO₄, filtered, and concentrated to dryness under reduced pressure. The residue was purified by prep-HPLC to give Compound 28 (3 mg, 5% yield) as a white solid. ¹H NMR (400 MHz, DMSO-d₆) δ 8.58 (s, 1H), 8.20 (s, 1H), 8.08 (s, 1H), 7.42 (s, 1H), 7.27 (t, J=7.9 Hz, 2H), 6.93-6.86 (m, 3H), 4.40 (d, J=5.7 Hz, 1H), 4.38-4.31 (m, 1H), 3.98-3.84 (m, 7H), 3.78 (dd, J=16.9, 5.0 Hz, 1H), 3.69 (d, J=11.2 Hz, 1H), 3.54 (d, J=10.8 Hz, 2H), 2.34-2.26 (m, 3H), 2.06-1.91 (m, 3H); LC/MS (ESI) m/z: 530 (M+H)⁺.

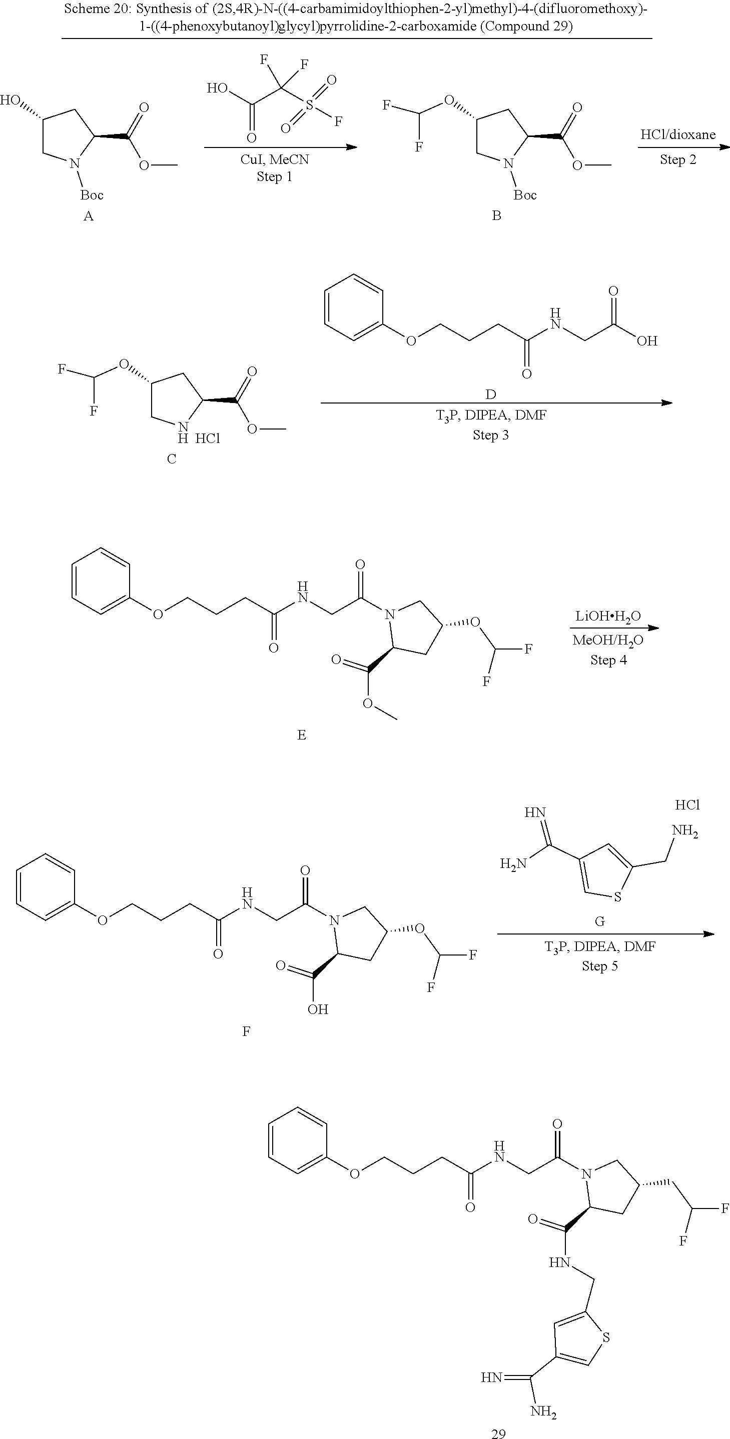

Step 1: 1-(tert-butyl) 2-methyl (2S,4R)-4-(difluoromethoxy)pyrrolidine-1,2-dicarboxylate (B). To a mixture of compound A (375 mg, 1.5 mmol) and CuI (60 mg, 0.31 mmol) in MeCN (5 mL) was added a solution of 2,2-difluoro-2-(fluorosulfonyl)acetic acid (330 mg, 1.8 mmol) in MeCN (1.5 mL) at 50° C. under N₂ atmosphere and the mixture was stirred at 50° C. overnight. The mixture was diluted with H₂O and extracted with EtOAc twice. The combined organic layers were washed with brine, dried over anhydrous Na₂SO₄, filtered, and concentrated to dryness under reduced pressure. The residue was purified by column chromatography on silica gel (DCM:MeOH=20:1) to give compound B (160 mg, 35.5% yield) as a yellow oil. LC/MS (ESI) m/z: 296 (M+H)⁺.

Step 2: methyl (2S,4R)-4-(difluoromethoxy)pyrrolidine-2-carboxylate (C). A mixture of compound B (60 mg, 0.20 mmol) and HCl/1,4-dioxane (5 mL) was stirred at room temperature for 1 hour. The reaction mixture was concentrated to dryness under reduced pressure to give compound C (35 mg, 87.5% yield) as a yellow oil, which was used directly in the next step. LC/MS (ESI) m/z: 196 (M+H)⁺.

Step 3: methyl (2S,4R)-4-(difluoromethoxy)-1-((4-phenoxybutanoyl)glycyl)pyrrolidine-2-carboxylate (E). To a mixture of compound C (35 mg, 0.18 mmol) and compound D (51 mg, 0.22 mmol) in DMF (5 mL) was added DIPEA (0.18 mL, 1.08 mmol) and T₃P (171 mg, 0.54 mmol) at 0° C. under N2 atmosphere and the mixture was stirred at 25° C. for 16 hours. The mixture was diluted with EtOAc and washed with saturated aq.NH₄Cl solution and brine. The organic layer was dried over anhydrous Na₂SO₄, filtered, and concentrated to dryness under reduced pressure. The residue was purified by column chromatography on silica gel (DCM:MeOH=97:3) to give compound E (35 mg, 47.3% yield) as a colorless oil. LC/MS (ESI) (m/z): 415 (M+H)⁺.

Step 4: (2S,4R)-4-(difluoromethoxy)-1-((4-phenoxybutanoyl)glycyl)pyrrolidine-2-carboxylic acid (F). To a solution of compound E (35 mg, 0.08 mmol) in MeOH (5 mL) and water (1 mL) was added LiOH·H₂O (3.6 mg, 0.08 mmol) at 0° C. and the mixture was stirred at room temperature for 16 hours. The mixture was acidified with 1 N aq. HCl to pH˜3 and extracted with EtOAc twice. The combined organic layers were dried over anhydrous Na₂SO₄, filtered, and concentrated to dryness under reduced pressure to give compound F (30 mg, 88.2% yield) as a yellow semi-solid, which was used directly in next step. LC/MS (ESI) (m/z): 401 (M+H)⁺.

Step 5: (2S,4R)-N-((4-carbamimidoylthiophen-2-yl)methyl)-4-(difluoromethoxy)-1-((4-phenoxybutanoyl)glycyl)pyrrolidine-2-carboxamide (Compound 29). To a mixture of compound 6 (30 mg, 0.075 mmol) and compound F (18 mg, 0.11 mmol) in DMF (3 mL) was added DIPEA (0.07 mL, 0.45 mmol) and T₃P (143 mg, 0.23 mmol) at 0° C. under N2 atmosphere and the mixture was stirred at 25° C. for 16 hours. The mixture was diluted with H₂O and extracted with CHCl₃/i-PrOH (V/V=3/1) twice. The organic layer was dried over anhydrous Na₂SO₄, filtered and concentrated to dryness under reduced pressure. The residue was purified by column chromatography on silica gel (DCM:MeOH=4:1) and further purified by prep-HPLC to give Compound 29 (11.2 mg, 28% yield) as a white solid. ¹H NMR (400 MHz, CD₃OD) δ 8.49 (s, 1H), 8.22 (dd, J=4.2, 1.5 Hz, 1H), 7.46 (d, J=21.9 Hz, 1H), 7.27-7.19 (m, 2H), 6.93-6.86 (m, 3H), 6.48 (dd, J=80.6, 68.2 Hz, 1H), 4.94 (dd, J=7.5, 4.1 Hz, 1H), 4.58 (ddd, J=22.9, 15.7, 7.9 Hz, 3H), 4.09-3.94 (m, 4H), 3.93-3.84 (m, 1H), 3.81-3.68 (m, 1H), 2.44 (ddd, J=18.0, 9.1, 5.1 Hz, 3H), 2.23-2.15 (m, 1H), 2.12-2.02 (m, 2H); LC/MS (ESI) m/z: 538 (M+H)⁺.

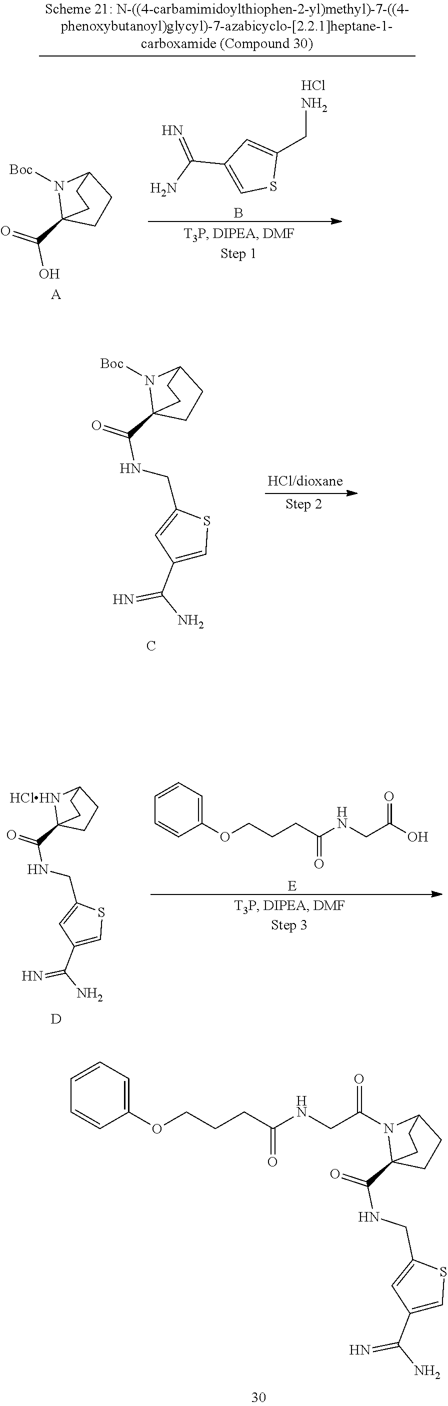

Step 1: tert-butyl 1-(((4-carbamimidoylthiophen-2-yl)methyl)carbamoyl)-7-azabicyclo-[2.2.1]heptane-7-carboxylate (C). To a mixture of compound A (150 mg, 0.62 mmol) and compound B (145 mg, 0.93 mmol) in DMF (5 mL) was added DIPEA (0.6 mL, 3.72 mmol) and T₃P (1.2 g, 1.86 mmol) at 0° C. under N₂ atmosphere and the mixture was stirred at 25° C. for 16 hours. The mixture was diluted with H₂O and extracted with CHCl₃/i-PrOH (V/V=3/1) twice. The combined organic layers were dried over anhydrous Na₂SO₄, filtered and concentrated to dryness under reduced pressure. The residue was purified by column chromatography on silica gel (DCM:MeOH=4:1) to give compound 3 (35 mg, 16.2% yield) as a white solid. LC/MS (ESI) (m/z): 379 (M+H)⁺.

Step 2: N-((4-carbamimidoylthiophen-2-yl)methyl)-7-azabicyclo[2.2.1]heptane-1-carboxamide (D). A mixture of compound C (35 mg, 0.092 mmol) and HCl/1,4-dioxane (5 mL) was stirred at room temperature for 1 hour. The reaction mixture was concentrated to dryness under reduced pressure to give compound D (25 mg, 96.2% yield) as a yellow oil, which was used directly in the next step. LC/MS (ESI) m/z: 279 (M+H)⁺.

Step 3: N-((4-carbamimidoylthiophen-2-yl)methyl)-7-((4-phenoxybutanoyl)glycyl)-7-azabicyclo[2.2.1]heptane-1-carboxamide (Compound 30). To a mixture of compound D (25 mg, 0.09 mmol) and compound E (26 mg, 0.11 mmol) in DMF (3 mL) was added DIPEA (0.09 mL, 0.54 mmol) and T₃P (171 mg, 0.27 mmol) at 0° C. under N₂ atmosphere and the mixture was stirred at 25° C. for 16 hours. The mixture was diluted with H₂O and extracted with CHCl₃/i-PrOH (V/V=3/1) twice. The combined organic layers were dried over anhydrous Na₂SO₄, filtered and concentrated to dryness under reduced pressure. The residue was purified by column chromatography on silica gel (DCM:MeOH=4:1) and further purified by prep-HPLC to give Compound 30 (2.0 mg, 4.5% yield) as a white solid. ¹H NMR (400 MHz, CD₃OD) δ 8.46 (s, 1H), 8.20 (d, J=1.6 Hz, 1H), 7.47 (s, 1H), 7.23 (dd, J=9.6, 6.5 Hz, 2H), 6.92-6.85 (m, 3H), 4.57 (s, 2H), 4.50 (t, J=4.7 Hz, 1H), 4.03-3.94 (m, 4H), 2.44 (t, J=7.4 Hz, 2H), 2.14-2.04 (m, 4H), 1.98 (t, J=11.7 Hz, 2H), 1.84-1.75 (m, 2H), 1.71-1.62 (m, 2H); LC/MS (ESI) m/z: 498 (M+H)⁺.

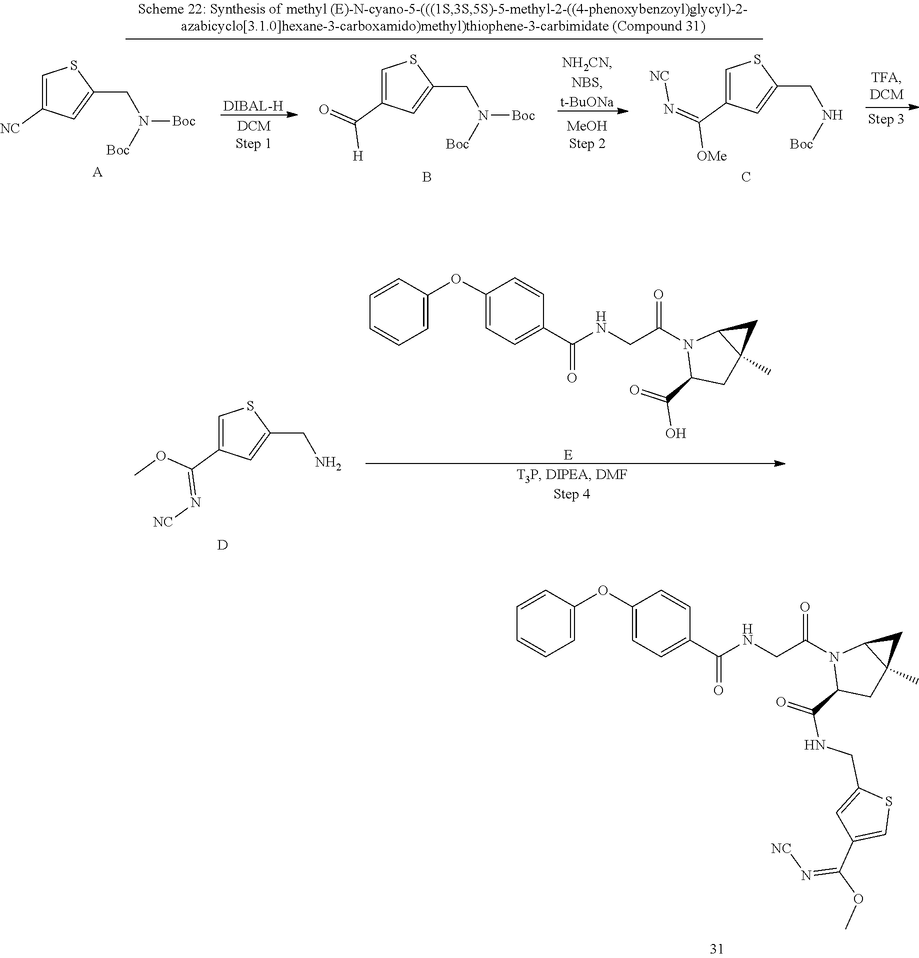

Step 1: tert-butyl N-(tert-butoxycarbonyl)-N-[(4-formylthiophen-2-yl)methyl]carbamate (B). To a solution of compound A (400 mg, 1.18 mmol) in DCM (7 mL) was added DIBAL-H (1.54 mL, 1.54 mmol) at −5° C. under N2 atmosphere and the mixture was stirred at −5° C. for 3 hours. The mixture was quenched with sat. NH₄Cl solution and stirred at room temperature overnight and extracted with DCM twice. The combined organic layers were washed with brine, dried over anhydrous Na₂SO₄, filtered and concentrated to dryness under reduced pressure. The residue was purified by column chromatography on silica gel (PE:EtOAc=50:1 to 5:1) to give compound B (74 mg, 18.3% yield) as a light oil. LC/MS (ESI) m/z: 342 (M+H)⁺.

Step 2: methyl (E)-5-(((tert-butoxycarbonyl)amino)methyl)-N-cyanothiophene-3-carbimidate (C). To a mixture of compound B (74 mg, 0.22 mmol) and NH₂CN (37 mg, 0.88 mmol) in MeOH (3 mL) was added t-BuONa (84 mg, 0.88 mmol) at room temperature under N2 atmosphere and the mixture was stirred at 30° C. for 30 minutes. NBS (156 mg, 0.88 mmol) was added to the mixture and the reaction was stirred at 50° C. overnight. The reaction was quenched with ice water and extracted with DCM twice. The combined organic layers were washed with brine, dried over anhydrous Na₂SO₄, and concentrated to dryness under reduced pressure. The residue was purified by prep-TLC (PE:EtOAc=1:1) to give compound C (35 mg, 53.8% yield) as a light oil. LC/MS (ESI) (m/z): 240 (M+H−56)⁺.

Step 3: methyl (Z)-5-(aminomethyl)-N-cyanothiophene-3-carbimidate (D). To a solution of compound C (35 mg, 0.12 mmol) in DCM (2.8 mL) was added TFA (0.8 mL) at 0° C. and the reaction was stirred at room temperature for 2 hours. The reaction mixture was concentrated to dryness under reduced pressure and dried under vacuum to give compound D (16 mg, 69.6% yield) as a yellow oil, which was used directly in the next step. LC/MS (ESI) m/z: 196 (M+H)⁺.

Step 4: methyl (E)-N-cyano-5-(((1S,3S,5S)-5-methyl-2-((4-phenoxybenzoyl)glycyl)-2-azabicyclo[3.1.0]hexane-3-carboxamido)methyl)thiophene-3-carbimidate (Compound 31). To a mixture of compound D (16 mg, 0.082 mmol) and compound E (39 mg, 0.098 mmol) in DMF (2 mL) was added DIPEA (0.09 mL, 0.49 mmol) and T₃P (156 mg, 0.25 mmol) at 0° C. under N2 atmosphere and the mixture was stirred at 25° C. for 16 hours. The mixture was diluted with EtOAc and washed with saturated aq. NaHCO₃ solution and brine. The organic layer was dried over anhydrous Na₂SO₄, filtered, and concentrated to dryness under reduced pressure. The residue was purified by prep-TLC (DCM:MeOH=20:1) to give Compound 31 (1.9 mg, 4.1% yield) as a white solid. ¹H NMR (400 MHz, DMSO-d₆) δ 8.66 (t, J=5.6 Hz, 1H), 8.60 (dd, J=6.0, 6.0 Hz, 1H), 8.55 (t, J=6.0 Hz, 1H), 7.91-7.87 (m, 2H), 7.59 (d, J=18.4 Hz, 1H), 7.48-7.43 (m, 2H), 7.22 (t, J=7.4 Hz, 1H), 7.12-7.08 (m, 2H), 7.06-7.02 (m, 2H), 4.70-4.63 (m, 1H), 4.51-4.33 (m, 3H), 4.03 (dd, J=16.8, 16.8 Hz, 1H), 3.95 (d, J=6.8 Hz, 3H), 3.44 (dd, J=6.0, 6.0 Hz, 1H), 2.29 (t, J=12.4 Hz, 1H), 1.99 (dd, J=13.2, 13.2 Hz, 1H), 1.23 (s, 3H), 1.18 (dd, J=5.2, 5.2 Hz, 1H), 0.68 (t, J=5.4 Hz, 1H); LC/MS (ESI) (m/z): 572 (M+H)⁺.

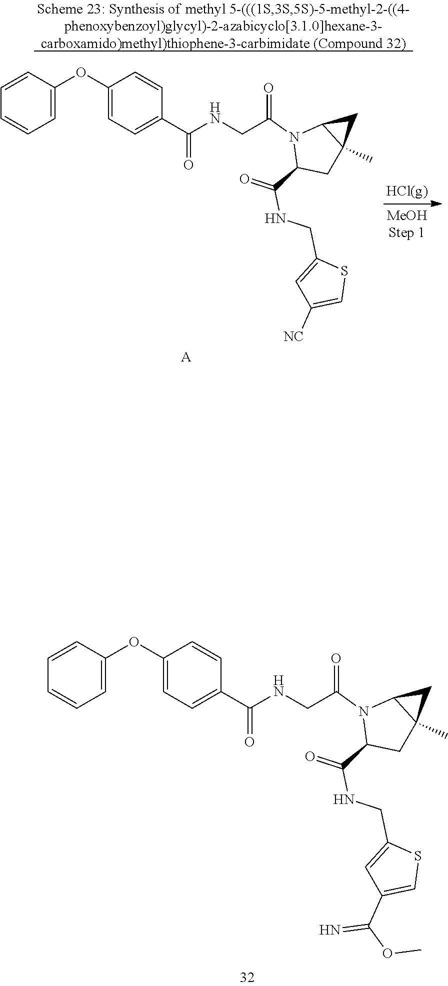

Step 1: methyl 5-(((1S,3S,5S)-5-methyl-2-((4-phenoxybenzoyl)glycyl)-2-azabicyclo[3.1.0]-hexane-3-carboxamido)methyl)thiophene-3-carbimidate (Compound 32). HCl gas (generated in situ from NaCl and conc. H₂SO₄) was passed through a solution of compound 1 (50 mg, 0.1 mmol) in MeOH (5 mL) and HCl gas flow was maintained for 0.5 hour at 25° C. The mixture was neutralized with aq.NaHCO₃ to pH=8 and extracted with CHCl₃/i-PrOH(v:v=3/1) twice. The combined organic layers were dried over anhydrous Na₂SO₄, filtered and concentrated to dryness under reduced pressure. The residue was purified by prep-HPLC to give Compound 32 (3 mg, 5.6% yield) as white solid. ¹H NMR (400 MHz, CD₃OD) δ 7.87-7.76 (m, 3H), 7.44-7.34 (m, 3H), 7.20 (t, J=7.4 Hz, 1H), 7.06 (dd, J=8.6, 0.9 Hz, 2H), 7.01-6.94 (m, 2H), 4.81 (d, J=3.3 Hz, 1H), 4.50 (s, 2H), 4.32 (dd, J=36.9, 16.5 Hz, 2H), 3.79 (s, 3H), 3.38 (d, J=3.6 Hz, 1H), 2.38 (d, J=13.0 Hz, 1H), 2.17 (dd, J=13.4, 3.3 Hz, 1H), 1.28 (s, 3H), 1.13 (dd, J=5.8, 2.4 Hz, 1H), 0.76 (t, J=5.4 Hz, 1H); LC/MS (ESI) m/z: 547 (M+H)⁺.

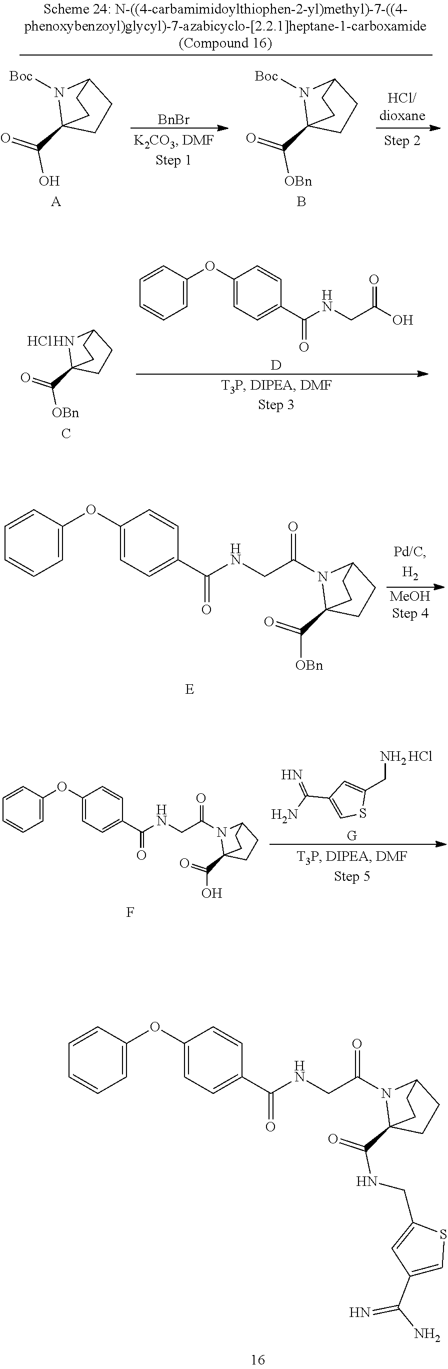

Step 1: 1-benzyl 7-(tert-butyl) 7-azabicyclo[2.2.1]heptane-1,7-dicarboxylate (B). To a solution of compound A (150 mg, 0.62 mmol) in DMF (5 mL) was added K₂CO₃ (257 mg, 1.86 mmol) and BnBr (160 mg, 1.86 mmol) at 0° C. under N2 atmosphere and the mixture was stirred at 25° C. for 16 hours. The mixture was diluted with water and extracted with EtOAc twice. The combined organic layers were washed with saturated aq.NH₄Cl solution and brine, dried over anhydrous Na₂SO₄, filtered and concentrated to dryness under reduced pressure. The residue was purified by column chromatography on silica gel (PE:EtOAc=5:1) to give compound B (200 mg, 97.1% yield) as a yellow oil. LC/MS (ESI) m/z: 332 (M+H)⁺.

Step 2: benzyl 7-azabicyclo[2.2.1]heptane-1-carboxylate (C). A mixture of compound B (200 mg, 0.60 mmol) and HCl/1,4-dioxane (5 mL) was stirred at room temperature for 1 hour. The reaction mixture was concentrated to dryness under reduced pressure to give compound C (130 mg, 93.5% yield) as a colorless oil, which was used directly in the next step. LC/MS (ESI) m/z: 232 (M+H)⁺.

Step 3: benzyl 7-((4-phenoxybenzoyl)glycyl)-7-azabicyclo[2.2.1]heptane-1-carboxylate (E). To a mixture of compound C (150 mg, 0.65 mmol) and compound D (211 mg, 0.78 mmol) in DMF (5 mL) was added DIPEA (0.64 mL, 3.9 mmol) and T₃P (1.2 g, 1.95 mmol) at 0° C. under N2 atmosphere and the mixture was stirred at 25° C. for 16 hours. The mixture was diluted with H₂O and extracted with EtOAc twice. The combined organic layers were dried over anhydrous Na₂SO₄, filtered and concentrated to dryness under reduced pressure. The residue was purified by column chromatography on silica gel (PE:EtOAc=3:2) to give compound E (205 mg, 65.3% yield) as a yellow solid. LC/MS (ESI) m/z: 485 (M+H)⁺.

Step 4: 7-((4-phenoxybenzoyl)glycyl)-7-azabicyclo[2.2.1]heptane-1-carboxylic acid (F). To a solution of compound E (205 mg, 0.42 mmol) in MeOH (5 mL) was added Pd/C (20 mg) under N2 atmosphere. The mixture was stirred at 25° C. for 1 hour. The mixture was filtered and the filtrate was concentrated to dryness under reduced pressure to give compound F (100 mg, 59.9% yield) as a white solid, which was used directly in the next step. LC/MS (ESI) (m/z): 395 (M+H)⁺.

Step 5: N-((4-carbamimidoylthiophen-2-yl)methyl)-7-((4-phenoxybenzoyl)glycyl)-7-azabicyclo[2.2.1]heptane-1-carboxamide (Compound 16). To a mixture of compound F (100 mg, 0.25 mmol) and compound G (59 mg, 0.38 mmol) in DMF (5 mL) was added DIPEA (0.25 mL, 1.5 mmol) and T₃P (242 mg, 0.75 mmol) at 0° C. under N2 atmosphere and the mixture was stirred at 25° C. for 16 hours. The mixture was diluted with H₂O and extracted with CHCl₃/i-PrOH(v:v=3/1) twice. The organic layer was dried over anhydrous Na₂SO₄, filtered and concentrated to dryness under reduced pressure. The residue was purified by column chromatography on silica gel (DCM:MeOH=5:1) and further purified by prep-HPLC to give Compound 16 (2.0 mg, 1.5% yield) as a white solid. ¹H NMR (400 MHz, CD₃OD) δ 8.50 (s, 1H), 8.19 (d, J=1.6 Hz, 1H), 7.87-7.80 (m, 2H), 7.47 (d, J=1.3 Hz, 1H), 7.44-7.38 (m, 2H), 7.20 (t, J=7.4 Hz, 1H), 7.09-7.04 (m, 2H), 7.03-6.98 (m, 2H), 4.59 (s, 3H), 4.19 (s, 2H), 2.19-1.97 (m, 4H), 1.83 (td, J=10.4, 3.4 Hz, 2H), 1.73-1.63 (m, 2H); LC/MS (ESI) m/z: 532 (M+H)⁺.

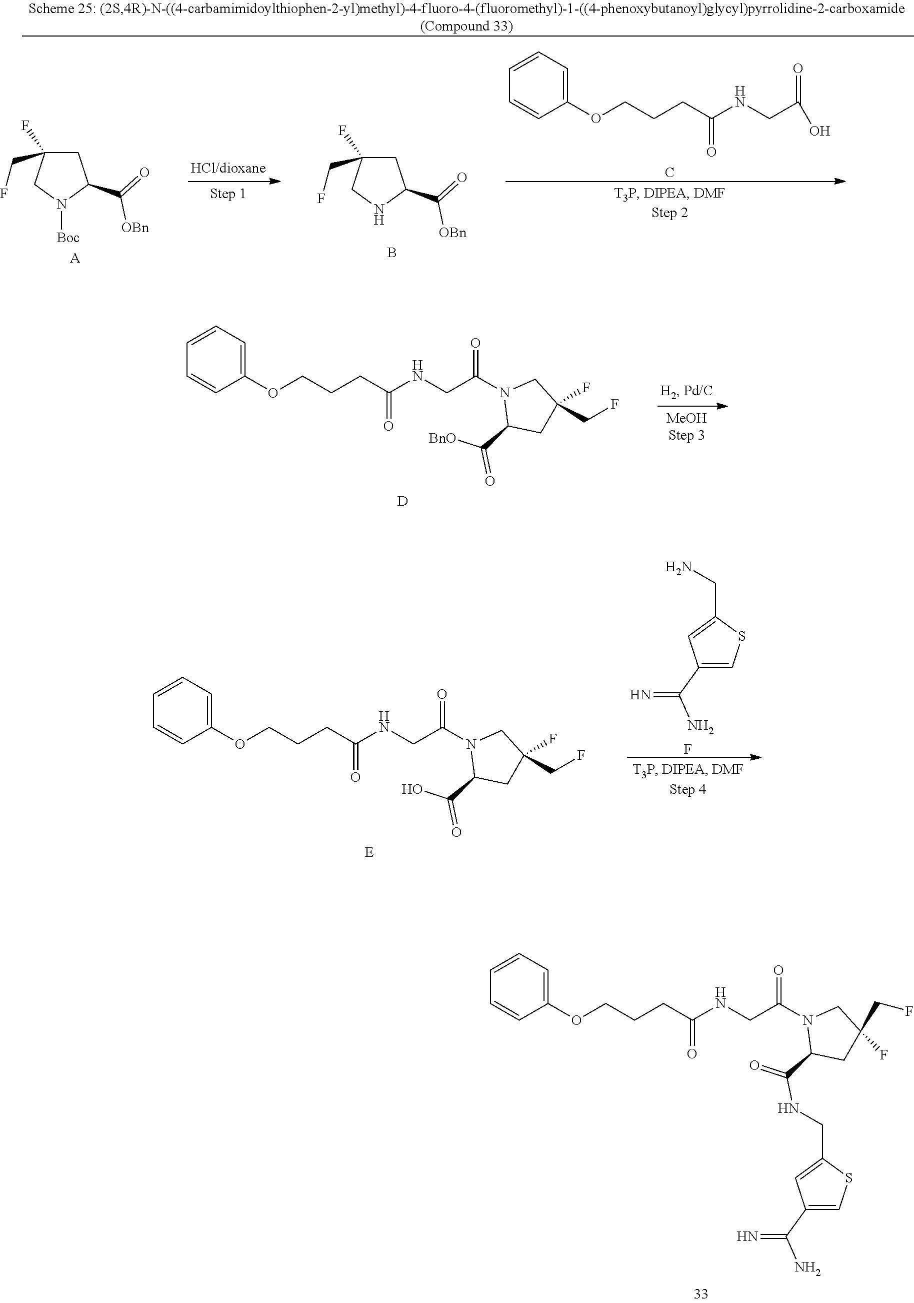

Step 1: benzyl (2S,4R)-4-fluoro-4-(fluoromethyl)pyrrolidine-2-carboxylate (B). A mixture of compound A (175 mg, 0.49 mmol) and HCl/1,4-dioxane (2 mL) was stirred at room temperature for 2 hours. The reaction mixture was concentrated to dryness under reduced pressure to give compound B (124 mg, 98% yield) as yellow oil, which was used directly in the next step. LC/MS (ESI) m/z: 256 (M+H)⁺.

Step 2: benzyl (2S,4R)-4-fluoro-4-(fluoromethyl)-1-((4-phenoxybutanoyl)glycyl)pyrrolidine-2-carboxylate (D). To a mixture of compound B (124 mg, 0.43 mmol) and compound C (101 mg, 0.43 mmol) in DMF (3 mL) was added DIPEA (0.4 mL, 2.58 mmol) and T₃P (820 mg, 1.29 mmol) at 0° C. under N2 atmosphere and the mixture was stirred at 30° C. for 16 hours. The mixture was diluted with water and extracted with EtOAc twice. The combined organic layers were washed with brine, dried over anhydrous Na₂SO₄, filtered, and concentrated to dryness under reduced pressure. The residue was purified by column chromatography on silica gel (DCM:MeOH=100:1 to 20:1) to give compound 3 (200 mg, 98% yield) as a yellow oil. LC/MS (ESI) m/z: 475 (M+H)⁺.

Step 3: (2S,4R)-4-fluoro-4-(fluoromethyl)-1-((4-phenoxybutanoyl)glycyl)pyrrolidine-2-carboxylic acid (E). To a solution of compound D (200 mg, 0.42 mmol) in MeOH (3 mL) was added 10% Pd/C (70 mg) at room temperature under H₂ atmosphere for 2 hours. The mixture was filtered and the filtrate was concentrated to dryness under reduced pressure to give compound E (162 mg, yield 100%) as a yellow oil, which was used directly in the next step. LC/MS (ESI) m/z: 385 (M+H)⁺.

Step 4: (2S,4R)-N-((4-carbamimidoylthiophen-2-yl)methyl)-4-fluoro-4-(fluoromethyl)-1-((4-phenoxybutanoyl)glycyl)pyrrolidine-2-carboxamide (Compound 33). To a mixture of compound E (50 mg, 0.13 mmol) and compound 5 (38 mg, 0.20 mmol) in DMF (1 mL) was added DIPEA (0.13 mL, 0.78 mmol) and T₃P (248 mg, 0.39 mmol) at 0° C. under N2 atmosphere and the mixture was stirred at 30° C. for 5 hours. The mixture was diluted with water and extracted with CH₃Cl/i-PrOH (v/v=3/1) twice. The combined organic layers were dried over anhydrous Na₂SO₄, filtered, and concentrated to dryness under reduced pressure. The residue was purified by prep-HPLC to give Compound 33 (1.4 mg, 2% yield) as a white solid. ¹H NMR (400 MHz, CD₃OD) δ 8.52 (s, 2H), 8.23 (dd, J=4.4, 4.4 Hz, 1H), 7.46 (t, J=9.0 Hz, 1H), 7.26-7.21 (m, 2H), 6.91-6.87 (m, 3H), 4.74-4.68 (m, 1H), 4.62 (dd, J=3.6, 5.6 Hz, 1H), 4.57 (d, J=8.0 Hz, 4H), 4.10-4.05 (m, 1H), 4.00 (dd, J=7.6, 8.8 Hz, 3H), 3.96-3.86 (m, 1H), 2.63-2.49 (m, 1H), 2.48-2.43 (m, 2H), 2.28-2.12 (m, 1H), 2.07 (t, J=7.2 Hz, 2H); LC/MS (ESI) m/z: 522 (M+H)⁺.

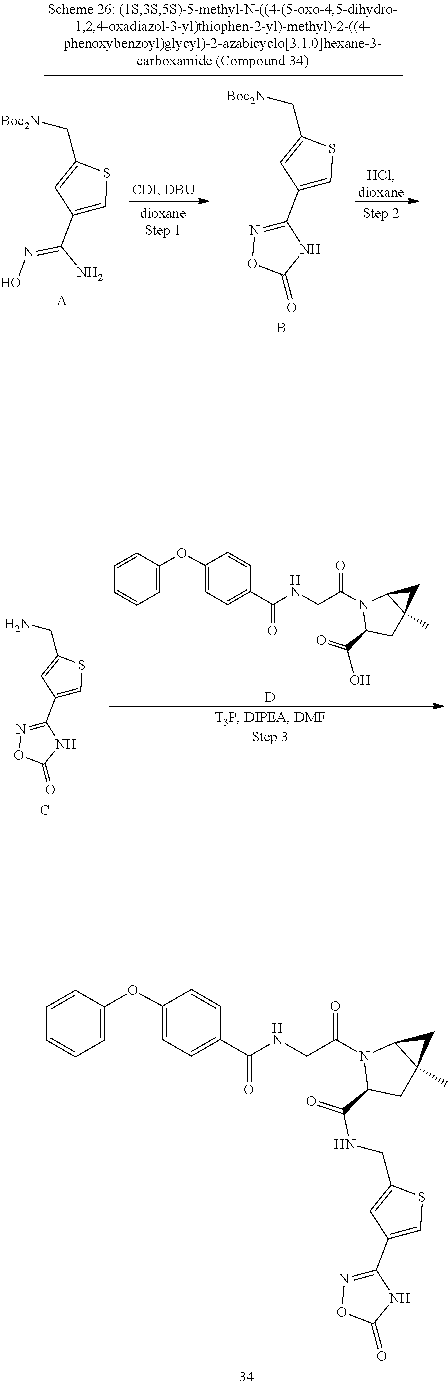

Step 1: tert-butyl N-(tert-butoxycarbonyl)-N-{[4-(5-oxo-4H-1,2,4-oxadiazol-3-yl)thiophen-2-yl]methyl}carbamate (B). To a solution of compound A (100 mg, 0.27 mmol) in 1,4-dioxane (1 mL) was added CDI (52 mg, 0.32 mmol) and DBU (45 mg, 0.3 mmol) at 0° C. under N₂ atmosphere and the mixture was stirred at 100° C. for 6 hours. The mixture was diluted with water and extracted with EtOAc twice. The combined organic layers were washed with brine, dried over anhydrous Na₂SO₄, filtered, and concentrated to dryness under reduced pressure. The residue was purified by column chromatography on silica gel (PE:EtOAc=1:1) to give compound B (56 mg, 52% yield) as a white solid. LC/MS (ESI) m/z: 242 (M−156+H)⁺.

Step 2: 3-(5-(aminomethyl)thiophen-3-yl)-1,2,4-oxadiazol-5(4H)-one (C). A mixture of compound B (56 mg, 0.14 mmol) and HCl/1,4-dioxane (2 mL) was stirred at room temperature for 2 hours. The reaction mixture was concentrated to dryness under reduced pressure to give compound C (27 mg, 97% yield) as a yellow solid, which was used directly in the next step. LC/MS (ESI) m/z: 198

Step 3: (1S,3S,5S)-5-methyl-N-((4-(5-oxo-4,5-dihydro-1,2,4-oxadiazol-3-yl)thiophen-2-yl)methyl)-2-((4-phenoxybenzoyl)glycyl)-2-azabicyclo[3.1.0]hexane-3-carboxamide (Compound 34). To a mixture of compound C (27 mg, 0.14 mmol) and compound D (55 mg, 0.14 mmol) in DMF (2 mL) was added DIPEA (0.14 mL, 0.84 mmol) and T₃P (267 mg, 0.42 mmol) at 0° C. under N2 atmosphere and the mixture was stirred at 30° C. for 16 hours. The mixture was diluted with water and extracted with CH₃Cl/i-PrOH (v/v=3/1) twice. The combined organic layers were dried over anhydrous Na₂SO₄, filtered and concentrated to dryness under reduced pressure. The residue was purified by prep-HPLC to give Compound 34 (1.5 mg, 2% yield) as a white solid. ¹HNMR (400 MHz, CD₃OD) δ 7.88-7.78 (m, 3H), 7.44-7.37 (m, 2H), 7.32 (d, J=1.2 Hz, 1H), 7.20 (t, J=7.6 Hz, 1H), 7.06 (dd, J=8.8, 0.9 Hz, 2H), 7.01-6.95 (m, 2H), 4.82 (d, J=3.3 Hz, 1H), 4.61-4.52 (m, 3H), 4.33 (dd, J=33.6, 37.2 Hz, 2H), 3.39 (dd, J=6.0, 6.0 Hz, 1H), 2.40 (t, J=12.4 Hz, 1H), 2.17 (dd, J=13.6, 13.2 Hz, 1H), 1.29 (s, 3H), 1.14 (dd, J=6.0, 5.6 Hz, 1H), 0.78 (t, J=5.4 Hz, 1H); LC/MS (ESI) m/z: 574 (M+H)⁺

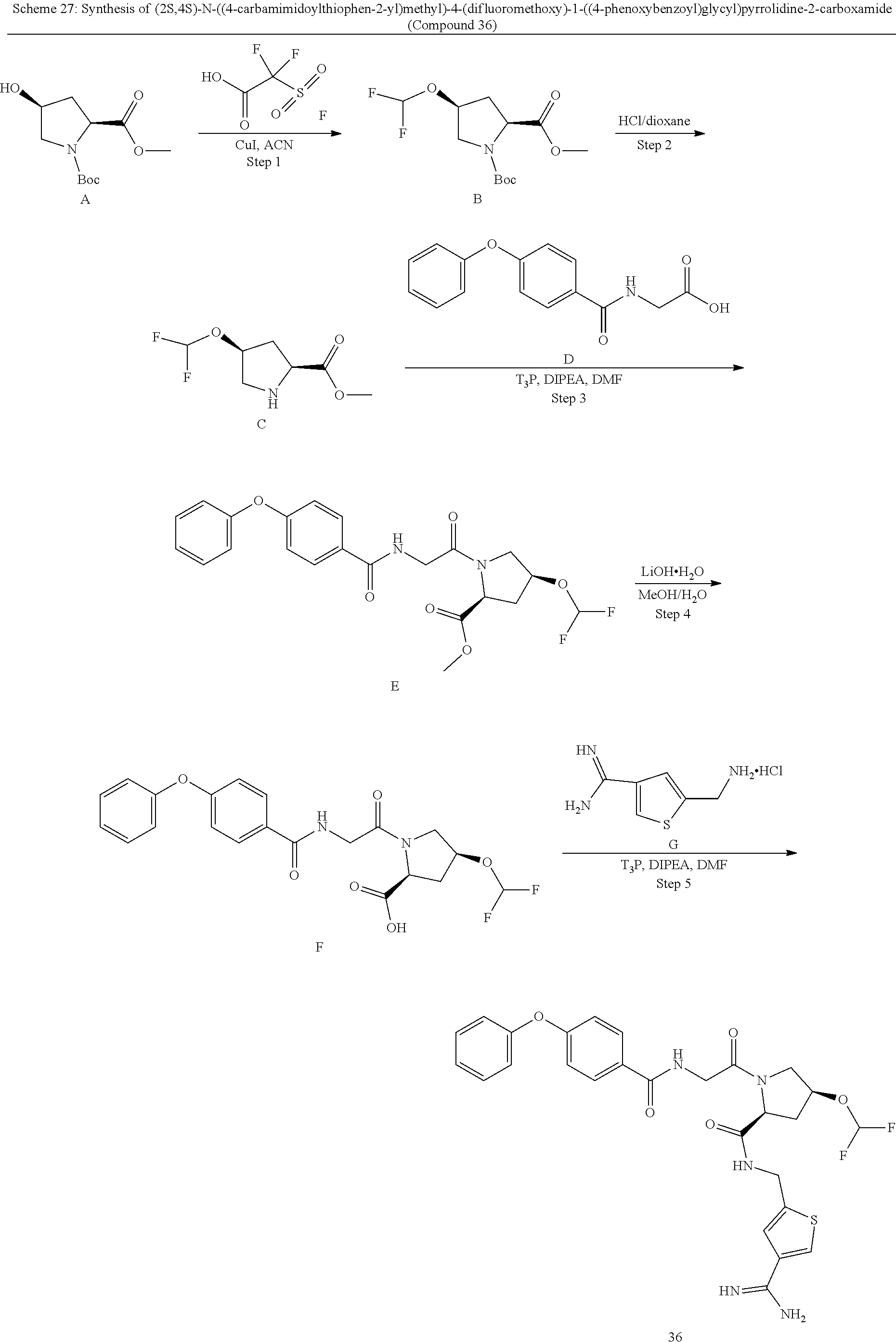

Step 1: 1-(tert-butyl) 2-methyl (2S,4S)-4-(difluoromethoxy)pyrrolidine-1,2-dicarboxylate (B). To a solution of compound A (750 mg, 3.0 mmol) in MeCN (10 mL) was added CuI (117 mg, 0.62 mmol) and the mixture was heated to 50° C. under N₂ atmosphere. A solution of 2,2-difluoro-2-(fluorosulfonyl)acetic acid (653 mg, 3.6 mmol) in MeCN (3.0 mL) was added dropwise to the mixture and the mixture was stirred at 50° C. overnight. The mixture was diluted with H₂O and extracted with EtOAc twice. The combined organic layers were washed with brine, dried over anhydrous Na₂SO₄, filtered, and concentrated to dryness under reduced pressure. The residue was purified by column chromatography on silica gel (DCM:MeOH=20:1) to give compound B (150 mg, 16.7% yield) as a yellow oil. LC/MS (ESI) m/z: 296 (M+H)⁺.

Step 2: methyl (2S,4S)-4-(difluoromethoxy)pyrrolidine-2-carboxylate (C). A mixture of compound B (150 mg, 0.51 mmol) and HCl/1,4-dioxane (3 mL) was stirred at room temperature for 1 hour. The reaction mixture was concentrated to dryness under reduced pressure to give compound C (95 mg, 96.0% yield) as a yellow oil, which was used directly in the next step. LC/MS (ESI) m/z: 196 (M+H)⁺.

Step 3: methyl (2S,4S)-4-(difluoromethoxy)-1-((4-phenoxybenzoyl)glycyl)pyrrolidine-2-carboxylate (E). To a mixture of compound C (95 mg, 0.49 mmol) and compound D (198 mg, 0.73 mmol) in DMF (5 mL) was added DIPEA (253 mg, 1.96 mmol) and T₃P (623 mg, 0.98 mmol, 50% in EtOAc) at 0° C. under N2 atmosphere and the mixture was stirred at 25° C. for 16 hours. The mixture was diluted with saturated aq. NH₄Cl solution and extracted with EtOAc twice. The combined organic layers were dried over anhydrous Na₂SO₄, filtered, and concentrated to dryness under reduced pressure. The residue was purified by column chromatography on silica gel (DCM:MeOH=100:1 to 20:1) to give compound E (190 mg, 87.0% yield) as a light-yellow oil. LC/MS (ESI) (m/z): 449 (M+H)⁺.

Step 4: (2S,4S)-4-(difluoromethoxy)-1-((4-phenoxybenzoyl)glycyl)pyrrolidine-2-carboxylic acid (F). To a solution of compound E (190 mg, 0.39 mmol) in MeOH (2 mL) and water (0.5 mL) was added LiOH·H₂O (17 mg, 0.39 mmol) at 0° C. and the mixture was stirred at room temperature for 16 hours. The mixture was acidified with 1 N aq. HCl to pH˜3 and concentrated to dryness under reduced pressure to give compound F (170 mg, 92.4% yield) as a yellow semi-solid, which was used directly in next step. LC/MS (ESI) (m/z): 435 (M+H)⁺.

Step 5: (2S,4S)-N-((4-carbamimidoylthiophen-2-yl)methyl)-4-(difluoromethoxy)-1-((4-phenoxybenzoyl)glycyl)pyrrolidine-2-carboxamide (Compound 36). To a mixture of compound F (50 mg, 0.115 mmol) and compound G (33 mg, 0.173 mmol) in DMF (3 mL) was added DIPEA (59 mg, 0.46 mmol) and T₃P (146 mg, 0.23 mmol, 50% in EtOAc) at 0° C. under N2 atmosphere and the mixture was stirred at 25° C. for 16 hours. The mixture was diluted with H₂O and extracted with CHCl₃/i-PrOH (v:v=3/1) twice. The combined organic layers were dried over anhydrous Na₂SO₄, filtered, and concentrated to dryness under reduced pressure. The residue was purified by column chromatography on silica gel (DCM:MeOH=4:1) and further purified by prep-HPLC to give Compound 36 (2.0 mg, 3.1% yield) as a white solid. ¹H NMR (400 MHz, CD₃OD) δ 8.53 (s, 1H), 8.22 (dd, J=16.3, 1.4 Hz, 1H), 7.86 (t, J=8.3 Hz, 2H), 7.45-7.39 (m, 2H), 7.22 (t, J=7.4 Hz, 1H), 7.08 (d, J=7.7 Hz, 2H), 7.02 (dd, J=8.5, 6.4 Hz, 2H), 6.38 (dd, J=90.5, 58.4 Hz, 1H), 4.75 (d, J=7.9 Hz, 1H), 4.68-4.56 (m, 3H), 4.29 (d, J=16.6 Hz, 1H), 4.15-3.99 (m, 2H), 3.91 (d, J=11.1 Hz, 1H), 2.57-2.50 (m, 1H), 2.38-2.35 (m, 1H); LC/MS (ESI) m/z: 572 (M+H)⁺.

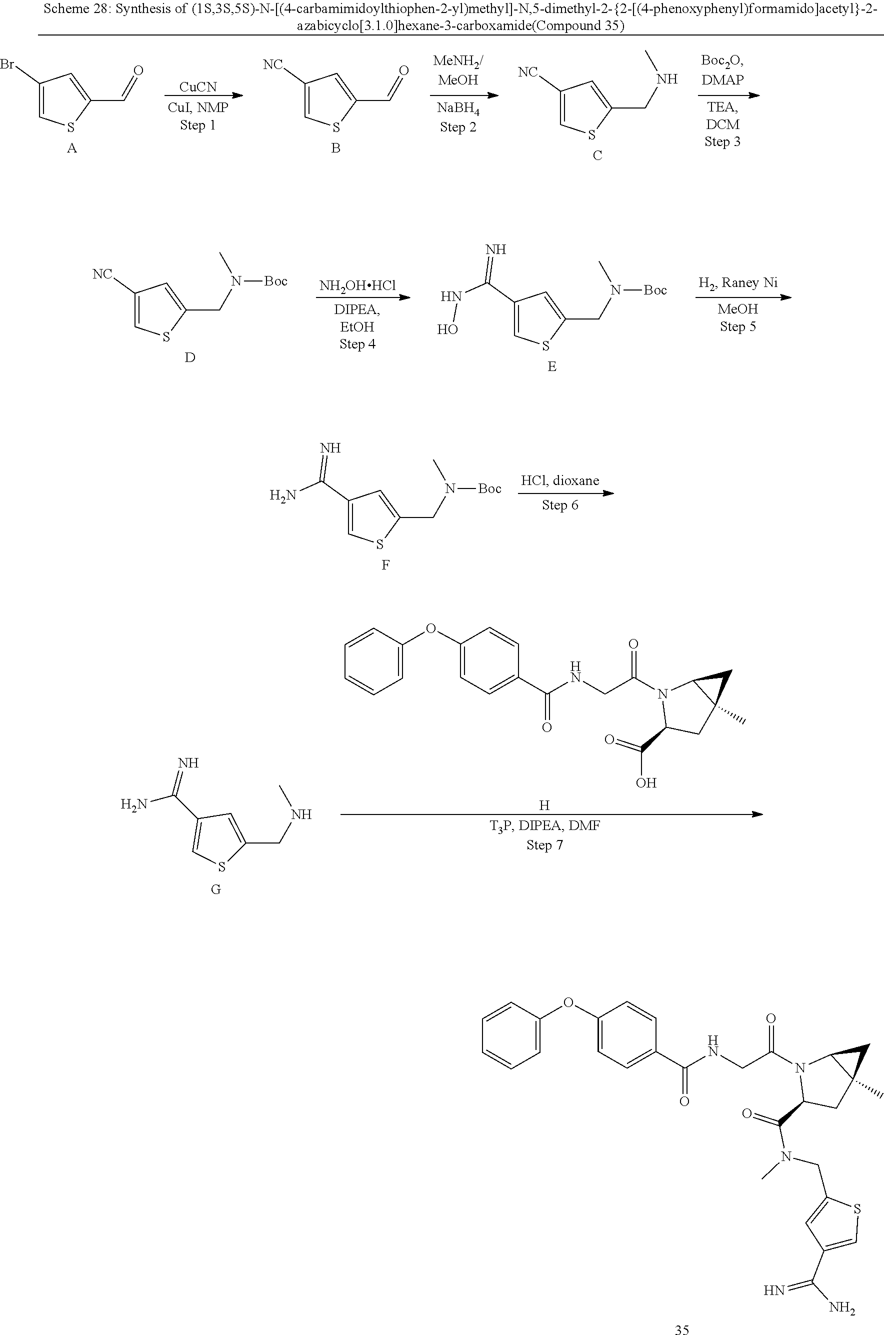

Step 1: 5-formylthiophene-3-carbonitrile(B). To a solution of compound A (10 g, 52.3 mmol) in NMP (200 mL) was added cuprous cyanide (14 g, 159.6 mmol) and cuprous iodide (4.5 g, 23.6 mmol) under N₂ atmosphere. The mixture was heated to 150° C. for 8 hours and monitored by TLC (petroleum ether:ethyl acetate=3:1). The resulting mixture was partitioned between ethyl acetate and saturated aqueous NH₄Cl. The organic layer was washed with brine, dried over anhydrous Na₂SO₄ and concentrated to dryness under reduced pressure to give the crude product, which was further purified by column chromatography (silica gel: 20-50% ethyl acetate in petroleum ether) to afford the title compound B (1.3 g, 40% yield) as a white solid. ¹H-NMR (400 MHz, CDCl₃) δ 9.95 (d, J=1.2 Hz, 1H), 8.27 (s, 1H), 7.94 (d, J=1.2 Hz, 1H).

Step 2: 5-[(methylamino)methyl]thiophene-3-carbonitrile (C). To a solution of compound 2 (1.1 g, 8.0 mmol), MeNH₂/MeOH solution (1.9 g, 16.0 mmol) and MgSO₄ (200 mg) in methanol were added followed by NaBH₄ (610 mg, 16.0 mmol) at 0° C. under N2 atmosphere. The mixture was stirred at room temperature for 2 hours and monitored by TLC (dichloromethane:methanol=20:1), and the resulting mixture was partitioned between ethyl acetate and saturated aqueous NH₄Cl. The organic layer was washed with brine, dried over anhydrous Na₂SO₄ and concentrated to dryness under reduced pressure to give crude product, which was further purified by column chromatography (silica gel: 10-30% methanol in dichloromethane) to afford compound C (710 mg, 4.6 mmol, 58% yield) as a white solid. LC/MS (ESI) m/z: 153 (M+H)⁺.

Step 3: tert-butyl N-[(4-cyanothiophen-2-yl)methyl]-N-methylcarbamate (D). To a solution of compound C (710 mg, 4.6 mmol) in dichloromethane (10 mL), TEA (1.4 g, 14.0 mmol), DMAP (171 mg, 1.4 mmol), and Boc₂₀ (1.5 g, 7.0 mmol) was added. The mixture was stirred at room temperature overnight and monitored by TLC (petroleum ether:ethyl acetate=5:1), and the resulting mixture was concentrated under reduced pressure to give crude product, which was further purified by column chromatography (silica gel: 10-20% ethyl acetate in petroleum ether) to afford compound D (1.12 g, 1.3 mmol, 95% yield) as a yellow oil. LC/MS (ESI) m/z:253 (M+H)⁺.

Step 4: tert-butyl N-{[4-(N-hydroxycarbamimidoyl)thiophen-2-yl]methyl}-N-methyl carbamate (E). To a solution of compound D (1.1 g, 4.439 mmol) and DIPEA (1.7 g, 13.3 mmol) in ethanol (10 mL) was added hydroxylamine hydrochloride (771 mg, 11.1 mmol). The mixture was stirred at room temperature overnight and monitored by TLC (dichloromethane:methanol=20:1), and the resulting mixture was concentrated under reduced pressure to give the crude product, which was further purified by column chromatography (silica gel: 5-15% methanol in dichloromethane) to afford compound E (1.7 g, 96.7% yield) as a white solid. LC/MS (ESI) m/z: 286 (M+H)⁺.

Step 5: tert-butyl N-[(4-carbamimidoylthiophen-2-yl)methyl]-N-methylcarbamate (F). To a solution of compound E (825 mg, 2.9 mmol) in methanol was added Raney Ni under H₂ atmosphere.

The mixture was heated to 30° C. and stirred for 3 hours. The resulting mixture was filtered and concentrated under reduced pressure to give the crude product, which was further purified by column chromatography (silica gel: 10-25% methanol in dichloromethane) to afford the compound F (610 mg, 78% yield) as a yellow solid. LC/MS (ESI) m/z: 270 (M+H)⁺.

Step 6: 5-[(methylamino)methyl]thiophene-3-carboximidamide (G). To a solution of compound F (305 mg, 1.1 mmol) in 1,4-dioxane was added HCl/1,4-dioxane (5 mL). The mixture was stirred at room temperature for 2 hours and monitored by TLC (dichloromethane:methanol=10:1), and the resulting mixture was concentrated under reduced pressure to afford the compound G (180 mg, 1.064 mmol, 94% yield) as a white solid which was used in next step without further purification. LC/MS (ESI) m/z: 170 (M+H)⁺.

Step 7: (1S,3S,5S)-N-[(4-carbamimidoylthiophen-2-yl)methyl]-N,5-dimethyl-2-{2-[(4-phenoxyphenyl)formamido]acetyl}-2-azabicyclo[3.1.0]hexane-3-carboxamide (Compound 35). To a solution of compound G (25 mg, 0.152 mmol) and (1S,3S,5S)-5-methyl-2-{2-[(4-phenoxyphenyl)formamido]acetyl}-2-azabicyclo[3.1.0]hexane-3-carboxylic acid (compound H; 30 mg, 0.076 mmol) in DMF (2 mL) was added DIPEA (59 mg, 0.456 mmol) and T₃P (145 mg, 0.228 mmol). The mixture was stirred at room temperature overnight and monitored by TLC (dichloromethane:methanol=10:1), and the resulting mixture was concentrated under reduced pressure to give the crude product, which was further purified by column chromatography (silica gel: 5-15% methanol in dichloromethane) to afford the Compound 35 (4 mg, 0.007 mmol, 9.6% yield) as a white solid. LC/MS (ESI) m/z:546 (M+H)⁺; ¹H-NMR (400 MHz, MeOD) δ 8.52 (s, 1H), 8.28 (dd, J=17.3, 4.7 Hz, 1H), 7.88 (d, J=8.8 Hz, 2H), 7.42 (dd, J=13.0, 5.3 Hz, 3H), 7.20 (t, J=7.4 Hz, 1H), 7.06 (d, J=7.8 Hz, 2H), 7.01 (d, J=8.8 Hz, 2H), 5.24 (dd, J=11.5, 3.8 Hz, 1H), 4.74 (s, 1H), 4.66 (s, 1H), 4.57 (d, J=16.7 Hz, 1H), 4.22 (d, J=16.8 Hz, 1H), 3.43 (dt, J=7.5, 3.8 Hz, 1H), 3.18-3.01 (m, 3H), 2.55 (t, J=12.0 Hz, 1H), 2.04-1.92 (m, 1H), 1.38 (dd, J=5.4, 2.4 Hz, 1H), 1.31 (d, J=2.9 Hz, 3H), 0.80 (t, J=5.2 Hz, 1H).

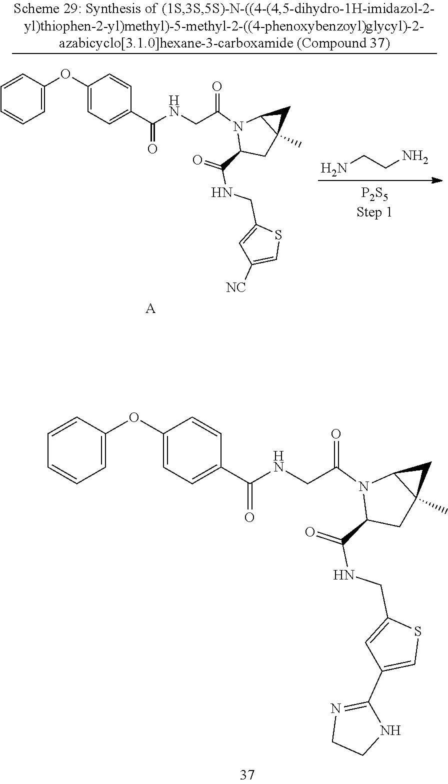

Step 1: (1S,3S,5S)-N-((4-(4,5-dihydro-1H-imidazol-2-yl)thiophen-2-yl)methyl)-5-methyl-2-((4-phenoxybenzoyl)glycyl)-2-azabicyclo[3.1.0]hexane-3-carboxamide (Compound 37). To a mixture of compound A (50.0 mg, 0.097 mmol) in ethane-1,2-diamine (2 mL) was added P₂S5 (6.5 mg, 0.029 mmol) at 0° C. under N2 atmosphere and the mixture was stirred at 120° C. for 4 hours. The mixture was diluted with H₂O and extracted with CHCl₃/i-PrOH(v:v=3/1) twice. The combined organic layers were dried over anhydrous Na₂SO₄, filtered and concentrated to dryness under reduced pressure. The residue was purified by column chromatography on silica gel (DCM:MeOH=4:1) to give Compound 37 (50 mg, 92.3% yield), 20 mg of which was further purified by prep-HPLC to give the compound (7.3 mg) as a white solid. ¹H NMR (400 MHz, MeOD) δ 8.55 (s, 1H), 8.18 (d, J=1.5 Hz, 1H), 7.85-7.81 (m, 2H), 7.44-7.40 (m, 2H), 7.36 (d, J=1.3 Hz, 1H), 7.24-7.18 (m, 1H), 7.07 (t, J=1.6 Hz, 1H), 7.06-7.05 (m, 1H), 7.00 (d, J=2.0 Hz, 1H), 6.99-6.98 (m, 1H), 4.83 (d, J=3.4 Hz, 1H), 4.55 (d, J=3.7 Hz, 2H), 4.34 (d, J=9.5 Hz, 2H), 3.92 (s, 4H), 3.41 (dd, J=6.0, 2.4 Hz, 1H), 2.56-2.34 (m, 1H), 2.17 (dd, J=13.4, 3.4 Hz, 1H), 1.30 (s, 3H), 1.15 (dd, J=5.8, 2.4 Hz, 1H), 0.80 (dd, J=5.9, 4.7 Hz, 1H). LC/MS (ESI) m/z: 558 (M+H)⁺.

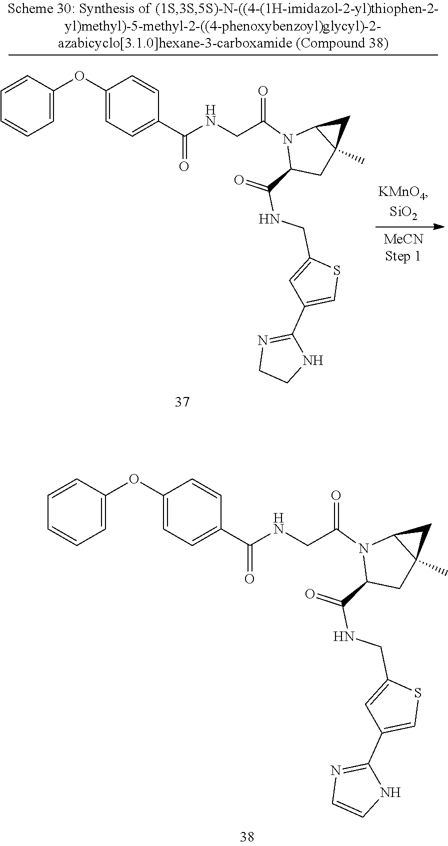 [SMILES: C[C@@]12C[C@@H]1N(C(=O)CNC(=O)c1ccc(Oc3ccccc3)cc1)[C@H](C(=O)NCc1cc(C3=NCCN3)cs1)C2 ; C[C@@]12C[C@@H]1N(C(=O)CNC(=O)c1ccc(Oc3ccccc3)cc1)[C@H](C(=O)NCc1cc(-c3ncc[nH]3)cs1)C2]

Step 1: (1S,3S,5S)-N-((4-(1H-imidazol-2-yl)thiophen-2-yl)methyl)-5-methyl-2-((4-phenoxy benzoyl)glycyl)-2-azabicyclo[3.1.0]hexane-3-carboxamide (Compound 38). To a mixture of Compound 37 (20.0 mg, 0.036 mmol) in acetonitrile (2 mL) was added KMnO₄ (17.0 mg, 0.108 mmol) and SiO₂ (28.7 mg, 800 mg per mmol), and the mixture was stirred at room temperature overnight. The mixture was filtered and concentrated to dryness under reduced pressure. The residue was purified by prep-HPLC to give Compound 38 (3.5 mg, 17.6% yield) as a white solid. ¹H NMR (400 MHz, MeOD) δ 7.84 (dd, J=9.1, 5.6 Hz, 2H), 7.66 (d, J=1.4 Hz, 1H), 7.43 (t, J=8.0 Hz, 3H), 7.22 (t, J=7.4 Hz, 1H), 7.11-6.92 (m, 6H), 4.84 (d, J=3.2 Hz, 1H), 4.56 (s, 2H), 4.44-4.27 (m, 2H), 3.46-3.35 (m, 1H), 2.42 (t, J=12.3 Hz, 1H), 2.21 (dd, J=13.4, 3.3 Hz, 1H), 1.31 (s, 3H), 1.18 (dd, J=5.7, 2.5 Hz, 1H), 0.78 (t, J=6.1 Hz, 1H).LC/MS (ESI) m/z: 556 (M+H)⁺.

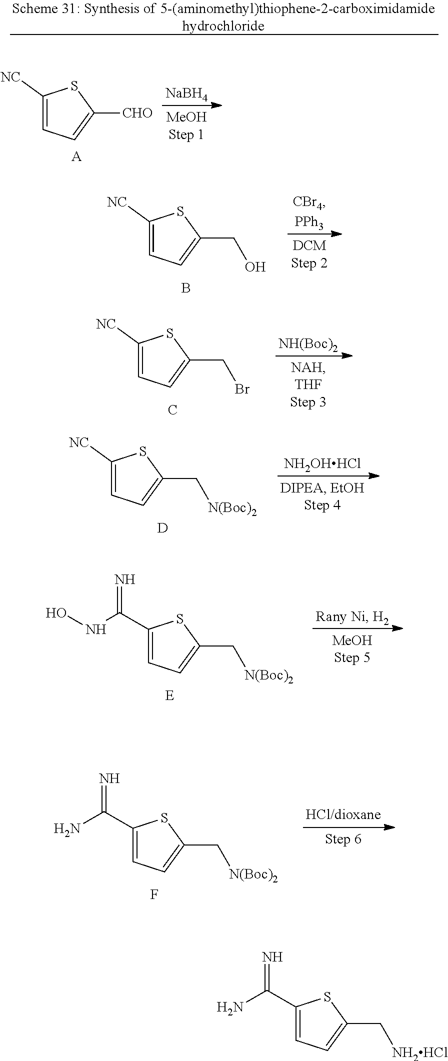

Step 1: 5-(hydroxymethyl)thiophene-2-carbonitrile (B). To a solution of compound A (500 mg, 3.64 mmol) in MeOH (5 mL) was added NaBH₄ (179 mg, 4.74 mmol) at 0° C. The mixture was stirred at room temperature for 1 hour. The mixture was diluted with H₂O and extracted with dichloromethane twice. The combined organic layers were washed with brine, dried over anhydrous Na₂SO₄, filtered and concentrated to dryness under reduced pressure. The residue was purified by column chromatography on silica gel (petroleum ether:ethyl acetate=2:1) to give compound A (428 mg, 84.8% yield) as colorless oil. LC/MS (ESI) m/z: 140 (M+H)⁺.

Step 2: 5-(bromomethyl)thiophene-2-carbonitrile (C). To a solution of compound B (428 mg, 3.08 mmol) in dichloromethane (5 mL) was added CBr₄ (1.1 g, 3.38 mmol) and PPh₃ (893 mg, 3.38 mmol) at 0° C. The mixture was stirred at room temperature under N2 atmosphere overnight. The mixture was diluted with water and extracted with dichloromethane twice. The combined organic layers were washed with brine, dried over anhydrous Na₂SO₄, filtered, and concentrated to dryness under reduced pressure. The residue was purified by column chromatography on silica gel (petroleum ether:ethyl acetate=4:1) to give compound C (430 mg, 69.2% yield) as a colorless oil. LC/MS (ESI) m/z: 203 (M+H)⁺.

Step 3: tert-butyl N-(tert-butoxycarbonyl)-N-{[5-(N-hydroxycarbamimidoyl) thiophen-2-yl]methyl}carbamate (D). To a solution of di-tert-butyl iminodicarboxylate (600 mg, 2.77 mmol) in anhydrous THE (8 mL) was added sodium hydride (60%, 127 mg, 3.19 mmol) at 0° C. under N₂ atmosphere and the mixture was stirred at 0° C. for 40 minutes. Compound C (430 mg, 2.13 mmol) in THE (2 mL) was added to the above mixture and the resulting mixture was stirred at 25° C. for another 16 hours under N2 atmosphere. The mixture was quenched with saturated aq. NH₄Cl solution and extracted with ethyl acetate twice. The organic layer was washed with brine, dried over anhydrous Na₂SO₄, filtered, and concentrated to dryness under reduced pressure. The residue was purified by column chromatography via silica gel (eluted with petroleum ether:ethyl acetate=4:1) to give compound D (320 mg, 40.5% yield) as a colorless oil. LC/MS (ESI) m/z: 239 (M+H−100)⁺.

Step 4: tert-butyl N-(tert-butoxycarbonyl)-N-{[5-(N-hydroxycarbamimidoyl) thiophen-2-yl]methyl}carbamate (E). To a mixture of compound D (320 mg, 0.946 mmol) in ethanol (5 mL) was added N,N-diisopropylethylamine (366 mg, 2.84 mmol) and hydroxylamine hydrochloride (164 mg, 2.36 mmol) at 0° C. The mixture was stirred at 25° C. for 16 hours. After completion of the reaction, the mixture was diluted with dichloromethane and washed with water and brine, dried over anhydrous Na₂SO₄, and concentrated to dryness under reduced pressure. The residue was purified by column chromatography on silica gel (PE:EtOAc=2:1) to give compound E (200 mg, 57% yield) as a white solid. LC/MS (ESI) m/z: 372 (M+H)⁺.

Step 5: tert-butyl N-(tert-butoxycarbonyl)-N-[(5-carbamimidoylthiophen-2-yl)methyl]carbamate (F). To a solution of compound 5 (200 mg, 0.538 mmol) in methanol (3 mL) was added Raney-nickel (1 mL) and acetic acid (0.5 mL) and the mixture was stirred at 30° C. for 16 hours under a H₂ balloon. The mixture was filtered and the filtrate was concentrated to dryness under reduced pressure to give compound 6 (160 mg, 83.6% yield) as white solid. LC/MS (ESI) m/z: 356 (M+H)⁺.

Step 6: 5-(aminomethyl)thiophene-2-carboximidamide hydrochloride. A solution of compound F (160 mg, 0.45 mmol) in HCl/1,4-dioxane (3 mL) was stirred at 25° C. for 1 hour. The mixture was concentrated to dryness under reduced pressure to give the title compound (86 mg, 100% yield) as white solid, which was used directly in the next step without further purification. LC/MS (ESI) m/z: 156 (M+H)⁺.

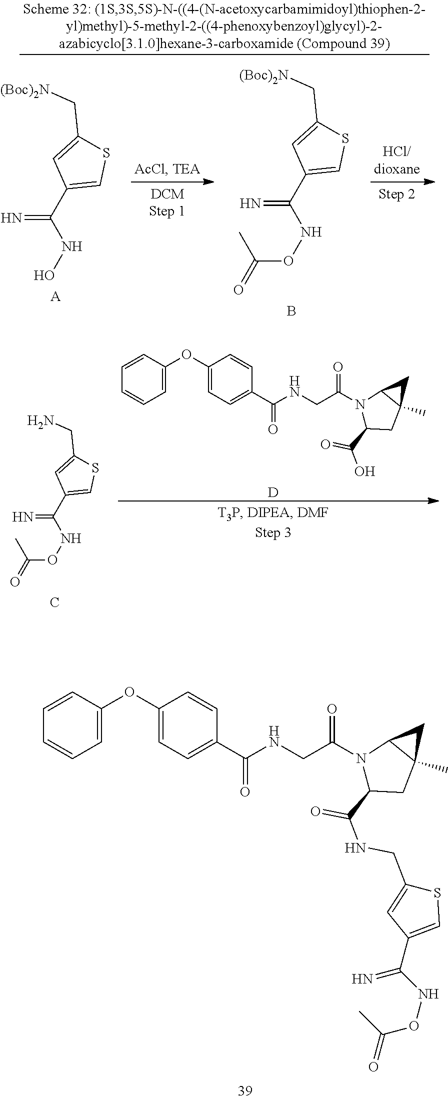

Step 1: (5-{[bis(tert-butoxycarbonyl)amino]methyl}thiophen-3-yl)methanimidamido acetate (B). To a solution of compound A (100 mg, 0.27 mmol) in DCM (3 mL) was added TEA (0.14 mL, 1.8 mmol) and AcCl (0.03 mL, 0.27 mmol) at 0° C. under N₂ atmosphere and the mixture was stirred at room temperature for 2 hours. The mixture was poured into ice water and extracted with DCM twice. The combined organic layers were washed with brine, dried over anhydrous Na₂SO₄, filtered and concentrated to dryness under reduced pressure. The residue was purified by column chromatography on silica gel (PE:EtOAc=100:1 to 1:1) to give compound B (80 mg, 71.8% yield) as a yellow oil. LC/MS (ESI) m/z: 358 (M−56+−H)⁺.

Step 2: N-acetoxy-5-(aminomethyl)thiophene-3-carboximidamide (C). A mixture of compound B (80 mg, 0.19 mmol) in HCl/1,4-dioxane (2 mL) was stirred at room temperature for 1 hour. The reaction mixture was concentrated to dryness under reduced pressure to give compound C (40 mg, 96.9% yield) as a yellow solid, which was used directly in the next step without further purification. LC/MS (ESI) m/z: 214 (M+H)⁺.

Step 3: (1S,3S,5S)-N-((4-(N-acetoxycarbamimidoyl)thiophen-2-yl)methyl)-5-methyl-2-((4-phenoxybenzoyl)glycyl)-2-azabicyclo[3.1.0]hexane-3-carboxamide (Compound 39). To a mixture of compound C (40 mg, 0.18 mmol) and compound D (74 mg, 0.18 mmol) in DMF (1 mL) was added DIPEA (0.19 mL) and T₃P (358 mg, 0.56 mmol) at 0° C. under N₂ atmosphere and the mixture was stirred at 35° C. for 5 hours. The mixture was diluted with EtOAc and water. The mixture was separated and the water layer was extracted with EtOAc twice. The combined organic layers were washed with brine, dried over anhydrous Na₂SO₄, filtered, and concentrated to dryness under reduced pressure. The residue was purified by prep-HPLC to give Compound 39 (7 mg, 6.33% yield) as white solid. ¹H NMR (400 MHz, CD₃OD) δ 7.87-7.82 (m, 2H), 7.79 (dd, J=7.6, 7.6 Hz, 1H), 7.44-7.38 (m, 2H), 7.35 (dd, J=5.2, 5.2 Hz, 1H), 7.22-7.17 (m, 1H), 7.09-7.03 (m, 2H), 7.02-6.97 (m, 2H), 4.81 (dd, J=11.2, 11.6 Hz, 1H), 4.56-4.49 (m, 2H), 4.39 (t, J=15.4 Hz, 1H), 4.19 (dd, J=57.6, 57.6 Hz, 1H), 3.37 (dd, J=10.0, 10.0 Hz, 1H), 2.44-2.26 (m, 1H), 2.23-2.10 (m, 4H), 1.27 (d, J=3.1 Hz, 3H), 1.16 (dd, J=5.6, 5.6 Hz, 1H), 0.76 (t, J=8.0 Hz, 1H); LC/MS (ESI) m/z: 590 (M+H)⁺.

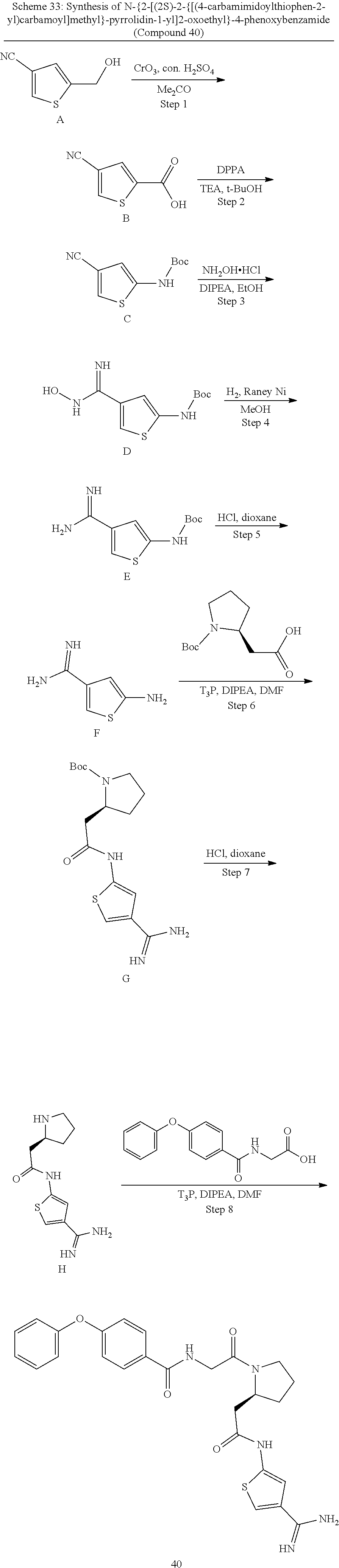

Step 1: 4-cyanothiophene-2-carboxylic acid (B). To a solution of compound A (2 g, 14.3 mmol) in acetone (20 mL) was added CrO₃ and concentrated H₂SO₄ (10 mL, 14.371 mmol) dropwise. The mixture was stirred at room temperature overnight and monitored by TLC (DCM:MeOH=20:1), the resulting mixture was partitioned between ethyl acetate and water. The organic layer was washed with brine, dried over anhydrous Na₂SO₄, and concentrated to dryness under reduced pressure to afford compound B (1.45 g, 9.467 mmol, 65% yield) as a white solid. LC/MS (ESI) m/z: 154 (M+H)⁺.

Step 2: tert-butyl N-(4-cyanothiophen-2-yl)carbamate (C). To a solution of compound B (900 mg, 5.8 mmol) and TEA (1.2 g, 11.752 mmol) in t-BuOH (10 mL) was added DPPA (3.2 g, 11.7 mmol) dropwise. The mixture was heated and stirred at 90° C. overnight and monitored by TLC (petroleum ether:ethyl acetate=5:1). The resulting mixture was partitioned between ethyl acetate and water. The organic layer was washed with brine, dried over anhydrous Na₂SO₄, and concentrated to dryness under reduced pressure to give the crude product, which was further purified by column chromatography (silica gel: 10-25% ethyl acetate in petroleum ether) to afford the compound C (810 mg, 61% yield) as a white solid. LC/MS (ESI) m/z: 225 (M+H)⁺.

Step 3: tert-butyl N-[4-(N-hydroxycarbamimidoyl)thiophen-2-yl]carbamate (D). To a solution of compound C (810 mg, 3.6 mmol) in EtOH (10 mL) was added DIPEA (1.4 g, 10.8 mmol) and hydroxylamine hydrochloride (627 mg, 9.0 mmol). The mixture was stirred at room temperature for 3 hours and monitored by TLC (DCM:MeOH=20:1), and the resulting mixture was partitioned between ethyl acetate and water. The organic layer was washed with brine, dried over anhydrous Na₂SO₄, and concentrated to dryness under reduced pressure to give the crude product, which was further purified by column chromatography (silica gel: 5-15% MeOH in DCM) to afford compound D (780 mg, 84% yield) as a white solid. LC/MS (ESI) m/z: 258 (M+H)⁺.

Step 4: tert-butyl N-(4-carbamimidoylthiophen-2-yl)carbamate (E). To a solution of compound D (780 mg, 3.031 mmol) in methanol (10 mL) was added acetic acid (0.1 mL) and Raney-Ni (1 mL in water). The mixture was heated and stirred at 40° C. for 2 hours and monitored by TLC (dichloromethane:methanol=10:1). The resulting mixture was concentrated to dryness under reduced pressure to give the crude product, which was further purified by column chromatography (silica gel: 10-30% methanol in dichloromethane) to afford compound E (710 mg, 97% yield) as a white solid. LC/MS (ESI) m/z: 242 (M+H)⁺.

Step 5: 5-aminothiophene-3-carboximidamide (F). To a solution of compound E (300 mg, 1.2 mmol) in 1,4-dioxane (2 mL) was added HCl/1,4-dioxane (2 mL). The mixture was stirred at room temperature for 2 hours and monitored by TLC (dichloromethane:methanol=10:1), and the resulting mixture was concentrated under reduced pressure to give compound F (160 mg, 91% yield) as a white solid. LC/MS (ESI) m/z: 142 (M+H)⁺.

Step 6: tert-butyl (2S)-2-{[(4-carbamimidoylthiophen-2-yl)carbamoyl]methyl}pyrrolidine-1-carboxylate (G). To a solution of compound F (160 mg, 1.1 mmol) and [(2S)-1-(tert-butoxycarbonyl)pyrrolidin-2-yl]acetic acid (519 mg, 2.3 mmol) in DMF (3 mL) was added DIPEA (878 mg, 6.8 mmol) and T₃P (2.1 g, 3.4 mmol). The mixture was stirred at room temperature for 2 hours and monitored by TLC (dichloromethane:methanol=20:1), the resulting mixture was partitioned between ethyl acetate and aq. NaHCO₃. The organic layer was washed with brine, dried over anhydrous Na₂SO₄, and concentrated under reduced pressure to give the crude product, which was further purified by column chromatography (silica gel: 5-15% methanol in dichloromethane) to afford compound G (100 mg, 25% yield) as a white solid. LC/MS (ESI) m/z: 353 (M+H)⁺.

Step 7: N-(4-carbamimidoylthiophen-2-yl)-2-[(2S)-pyrrolidin-2-yl]acetamide (H). To a solution of compound G (100 mg, 0.284 mmol) in 1,4-dioxane (2 mL) was added HCl/1,4-dioxane (1 mL). The mixture was stirred at room temperature for 2 hours and monitored by TLC (dichloromethane:methanol=10:1), the resulting mixture was concentrated under reduced pressure to give compound H (70 mg, 97% yield) as a white solid. LC/MS (ESI) m/z: 253 (M+H)⁺.

Step 8: N-{2-[(2S)-2-{[(4-carbamimidoylthiophen-2-yl)carbamoyl]methyl}pyrrolidin-1-yl]-2-oxoethyl}-4-phenoxybenzamide (Compound 40). To a solution of compound H (35 mg, 0.139 mmol) and [(4-phenoxyphenyl)formamido]acetic acid (75 mg, 0.277 mmol) in DMF (2 mL) was added DIPEA (107 mg, 0.832 mmol) and T₃P (264 mg, 0.416 mmol). The mixture was stirred at room temperature for 2 hours and monitored by TLC (dichloromethane:methanol=10:1), the resulting mixture was partitioned between (chloroform:isopropyl alcohol=3:1) and aq. NaHCO₃. The organic layer was washed with brine, dried over anhydrous Na₂SO₄ and concentrated to dryness under reduced pressure to give the crude product, which was further purified by column chromatography (silica gel: 10-20% methanol in dichloromethane) to afford Compound 40 (2 mg, 0.004 mmol, 2.8% yield) as a white solid. LC/MS (ESI) m/z:506 (M+H). ¹H NMR (400 MHz, MeOD) δ 7.84 (dd, J=21.1, 5.4 Hz, 3H), 7.42 (dd, J=8.5, 7.5 Hz, 2H), 7.20 (t, J=7.4 Hz, 1H), 7.14-7.04 (m, 3H), 7.03-6.99 (m, 2H), 4.48 (s, 1H), 4.22 (d, J=16.7 Hz, 1H), 4.10 (d, J=16.8 Hz, 1H), 3.70-3.53 (m, 2H), 2.95 (dd, J=14.4, 4.3 Hz, 1H), 2.77-2.50 (m, 1H), 2.14-2.01 (m, 3H), 1.91 (s, 1H).

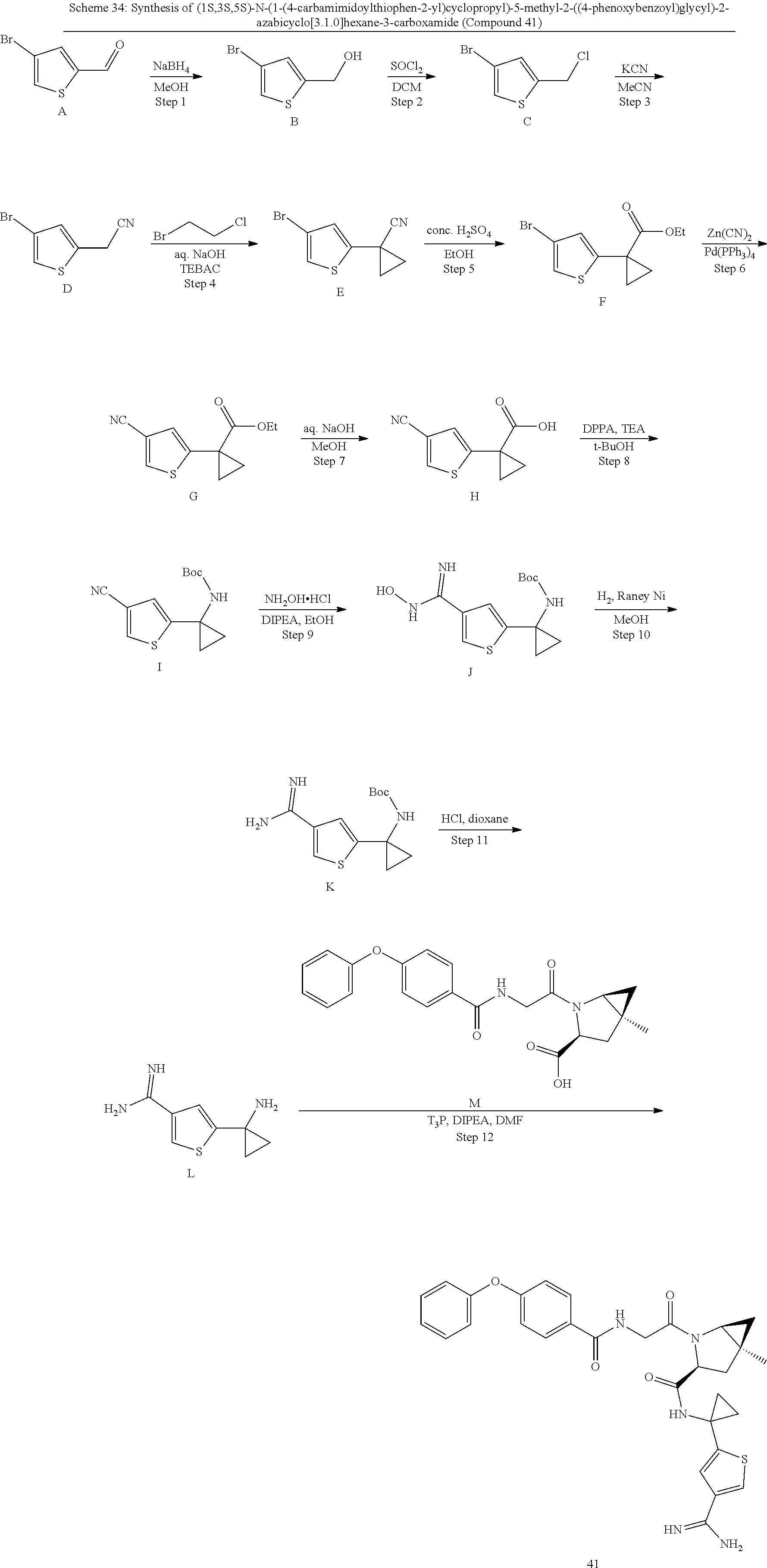

Step 1: Synthesis of (4-bromothiophen-2-yl)methanol (B). To a solution of compound A (55.7 g, 291.6 mmol) in MeOH (600 mL) was added NaBH₄ (22.2 g, 583.1 mmol) at 0° C. under N2 atmosphere. The reaction was stirred at room temperature overnight and quenched with sat. NH₄Cl. The mixture was extracted with ethyl acetate twice and the combined organic layers were separated, dried over anhydrous Na₂SO₄, concentrated to dryness under reduced pressure, and the residue was purified by column chromatography on silica gel (petroleum ether:ethyl acetate=2:1) to give compound B (55.6 g, 98.9% yield) as a white solid. ¹H NMR (400 MHz, CDCl₃) δ 7.17 (d, J=1.4 Hz, 1H), 7.17 (d, J=1.4 Hz, 1H), 6.95-6.87 (m, 1H), 6.96-6.85 (m, 1H), 4.77 (d, J=4.9 Hz, 2H), 4.77 (d, J=4.9 Hz, 2H), 2.13 (t, J=5.4 Hz, 1H), 2.13 (t, J=5.4 Hz, 1H).

Step 2: Synthesis of 4-bromo-2-(chloromethyl)thiophene (C). To a solution of compound B (11.0 g, 56.98 mmol) in dichloromethane (5 mL) was added SOCl₂ (13.5 g, 114.0 mmol) dropwise at 0° C. under N2 atmosphere. The reaction was stirred at room temperature overnight and quenched with saturated aqueous NH₄Cl. The resulting mixture was extracted with dichloromethane twice. The combined organic layers were washed with water and brine (2×100 mL), dried over anhydrous Na₂SO₄, filtered, and concentrated to dryness under reduced pressure. The residue was purified by column chromatography on silica gel (petroleum ether:ethyl acetate=5:1) to give compound C (8.9 g, 73% yield) as a yellow oil. ¹H NMR (400 MHz, CDCl₃) δ 7.21 (d, J=1.5 Hz, 1H), 7.06-6.96 (m, 1H), 4.73 (d, J=0.6 Hz, 2H).

Step 3: Synthesis of 2-(4-bromothiophen-2-yl)acetonitrile (D). To a solution of compound C (8.9 g, 42.08 mmol) in acetonitrile was added potassium cyanide (4.93 g, 75.74 mmol) at room temperature under N₂ atmosphere, and the reaction mixture was stirred at room temperature under N₂ atmosphere for 0.5 hour. The mixture was stirred at 85° C. overnight. The mixture was filtered and concentrated to dryness under reduced pressure, and purified by flash column chromatography (petroleum ether:ethyl acetate=20:1) to give compound D (4.8 g, 56% yield) as a yellow oil. ¹H NMR (400 MHz, CDCl₃) δ 7.18 (d, J=1.4 Hz, 1H), 6.99 (d, J=1.2 Hz, 1H), 3.88 (d, J=0.9 Hz, 2H).

Step 4: Synthesis of 1-(4-bromothiophen-2-yl)cyclopropane-1-carbonitrile (E). A mixture of compound D (3.0 g, 14.85 mmol), 1-bromo-2-chloroethane (3.2 g, 22.27 mmol), and benzyl triethyl ammonium chloride (68 mg, 0.3 mmol) was stirred at room temperature under N2 atmosphere for 0.5 hour. NaOH (3.56 g, 89.08 mmol) in water (4 mL) was added, and the mixture was stirred at 50° C. overnight and quenched with saturated aqueous NH₄Cl. The resulting mixture was extracted with ethyl acetate twice. The combined organic layers were washed with H₂O and brine, dried over anhydrous Na₂SO₄, filtered, and concentrated to dryness under reduced pressure. The residue was purified by flash column chromatography to give compound E (2.54 g, 75% yield) as a yellow oil. ¹H NMR (400 MHz, CDCl₃) δ 7.10 (d, J=1.5 Hz, 1H), 6.97 (d, J=1.5 Hz, 1H), 1.77 (q, J=5.2 Hz, 2H), 1.43 (q, J=5.3 Hz, 2H).

Step 5: Synthesis of ethyl 1-(4-bromothiophen-2-yl)cyclopropane-1-carboxylate (F). To a solution of compound E (2.4 g, 10.52 mmol) in ethanol (25 mL) was added conc. H₂SO₄ at 0° C. under N₂ atmosphere. The mixture was stirred at 90° C. for 16 hours. The mixture was quenched with H₂O and extracted with ethyl acetate twice. The combined organic layers were dried over anhydrous Na₂SO₄, filtered, and concentrated to dryness under reduced pressure. The residue was purified by column chromatography on silica gel (petroleum ether:ethyl acetate=20:1) to give compound F (1.6 g, yield 55%) as a yellow oil. ¹H NMR (400 MHz, DMSO) δ 7.09 (d, J=1.5 Hz, 1H), 6.83 (d, J=1.5 Hz, 1H), 4.15 (q, J=7.1 Hz, 2H), 1.71 (q, J=4.1 Hz, 2H), 1.30 (q, J=3.7 Hz, 2H), 1.23 (t, J=7.1 Hz, 3H).

Step 6: Synthesis of ethyl 1-(4-bromothiophen-2-yl)cyclopropane-1-carboxylate (G). To a solution of compound E (1.5 g, 5.45 mmol) in NMP (15 mL) was added zinc cyanide (1.28 g, 10.90 mmol) and Pd(PPh₃)₄ (315 mg, 0.27 mmol) at room temperature under N2 atmosphere, and the mixture was stirred at 160° C. for 3 hours. The mixture was quenched with ice water and organic layer was separated. The organic layer was washed with water, dried over anhydrous Na₂SO₄, filtered, and concentrated to dryness under reduced pressure. The residue was purified by column chromatography on silica gel (petroleum ether:ethyl acetate=5:1) to give compound G (1.1 g, yield 91%) as a yellow oil. ¹H NMR (400 MHz, CDCl₃) δ 7.80 (d, J=1.3 Hz, 1H), 7.04 (d, J=1.3 Hz, 1H), 4.16 (q, J=7.1 Hz, 2H), 1.77 (q, J=4.2 Hz, 2H), 1.33 (q, J=4.2 Hz, 2H), 1.23 (t, J=7.1 Hz, 3H).

Step 7: Synthesis of 1-(4-cyanothiophen-2-yl)cyclopropane-1-carboxylic acid (H). To a solution of compound G (1.1 g, 4.97 mmol) in MeOH (12 mL) was added NaOH (0.40 g, 9.94 mmol) in H₂O (1 mL) at room temperature under N2 atmosphere and the mixture was stirred at room temperature for 4 hours. The mixture was quenched with 4 N HCl and extracted with ethyl acetate twice. The combined organic layers were separated, washed with water, dried over anhydrous Na₂SO₄, filtered, and concentrated to dryness under reduced pressure. The residue was purified by column chromatography on silica gel (petroleum ether:ethyl acetate=1:1) to give compound G (820 mg, yield 85%) as a yellow solid. ¹H NMR (400 MHz, MeOD) δ 8.09 (s, 1H), 7.17 (s, 1H), 1.75 (q, J=4.1 Hz, 2H), 1.38 (q, J=4.1 Hz, 2H).

Step 8: Synthesis of tert-butyl (1-(4-cyanothiophen-2-yl)cyclopropyl)carbamate (1). To a solution of compound G (200 mg, 1.04 mmol) and TEA (314 mg, 3.11 mmol) in t-BuOH (2 mL) was added DPPA (428 mg, 1.55 mmol) at room temperature under N2 atmosphere and the mixture was stirred at 90° C. overnight. Water was added and the mixture was extracted with ethyl acetate twice. The combined organic layers were separated, washed with water, dried over anhydrous Na₂SO₄, filtered, and concentrated to dryness under reduced pressure. The residue was purified by column chromatography on silica gel (petroleum ether:ethyl acetate=3:1) to give compound I (100 mg, yield 36.5%) as a yellow solid. ¹H NMR (400 MHz, CDCl₃) δ 7.69 (d, J=0.8 Hz, 1H), 6.93 (s, 1H), 1.43 (s, 9H), 1.35 (s, 2H), 1.26 (d, J=4.9 Hz, 2H).

Step 9: Synthesis of tert-butyl (1-(4-(N-hydroxycarbamimidoyl)thiophen-2-yl) cyclopropyl)-carbamate (J). To a solution of compound I (100 mg, 0.38 mmol) and DIPEA (196 mg, 1.51 mmol) in methanol (2 mL) was added NH₂OH—HCl (50 mg, 0.76 mmol) at room temperature under N2 atmosphere, and the mixture was stirred at room temperature overnight. The mixture was concentrated to dryness under reduced pressure to give compound J (80 mg, yield 71.1%) as a yellow solid. ¹H NMR (400 MHz, MeOD) δ 7.48 (s, 1H), 7.02 (s, 1H), 1.45 (s, 9H), 1.26 (d, J=1.8 Hz, 2H), 1.23 (dd, J=3.4, 1.8 Hz, 2H).

Step 10: Synthesis of tert-butyl (1-(4-carbamimidoylthiophen-2-yl)cyclopropyl) carbamate (K). To a solution of compound J (75 mg, 0.25 mmol) in methanol (2 mL) was added Raney-Ni (0.5 mL) at room temperature under H₂ atmosphere and the mixture was stirred at room temperature for 4 hours. The mixture was filtered and concentrated to dryness under reduced pressure to give compound K (60 mg, yield 84.5%) as a yellow solid, which was used directly in the next step without further purification. (ESI) m/z: 282 (M+H)⁺

Step 11: Synthesis of 5-(1-aminocyclopropyl)thiophene-3-carboximidamide (L). To a solution of compound K (60 mg, 0.21 mmol) in methanol (1 mL) was added HCl in 1,4-dioxane (1.0 M) at room temperature under N₂ atmosphere, and the mixture was stirred at room temperature for 2 hours. The mixture was concentrated to dryness under reduced pressure to give compound L (40 mg, yield 100%) as a yellow solid, which was used directly in the next step without further purification. (ESI) m/z: 182 (M+H)⁺

Step 12: Synthesis of (1S,3S,5S)-N-(1-(4-carbamimidoylthiophen-2-yl)cyclopropyl)-5-methyl-2-((4-phenoxybenzoyl)glycyl)-2-azabicyclo[3.1.0]hexane-3-carboxamide (Compound 41) To a solution of (1 S,3S,5S)-5-methyl-2-((4-phenoxybenzoyl)glycyl)-2-azabicyclo[3.1.0]hexane-3-carboxylic acid (30 mg, 0.076 mmol) and compound K (40 mg, 0.23 mmol) in DMF (1 mL) was added DIPEA (40 mg, 0.304 mmol) and T₃P (42 mg, 0.152 mmol) at room temperature under N₂ atmosphere and the mixture was stirred at room temperature overnight. Water was added and the mixture was extracted with ethyl acetate twice and separated. The combined organic layers were concentrated to dryness under reduced pressure and purified by prep-HPLC to give Compound 41 (2.7 mg, yield 6.2%) as a yellow solid. ¹H NMR (400 MHz, MeOD) δ 8.11 (d, J=1.6 Hz, 1H), 7.90-7.85 (m, 2H), 7.45-7.39 (m, 2H), 7.31 (d, J=1.6 Hz, 1H), 7.22 (t, J=7.4 Hz, 1H), 7.07 (dd, J=8.6, 1.0 Hz, 2H), 7.02 (d, J=8.8 Hz, 2H), 4.79 (dd, J=11.4, 3.5 Hz, 1H), 4.40 (d, J=16.5 Hz, 1H), 4.29 (d, J=16.5 Hz, 1H), 3.41 (dd, J=5.9, 2.4 Hz, 1H), 2.41 (t, J=12.6 Hz, 1H), 2.13 (dd, J=13.3, 3.5 Hz, 1H), 1.36 (d, J=3.5 Hz, 2H), 1.31 (s, 5H), 1.18 (dd, J=5.7, 2.3 Hz, 1H), 0.81 (t, J=5.3 Hz, 1H).

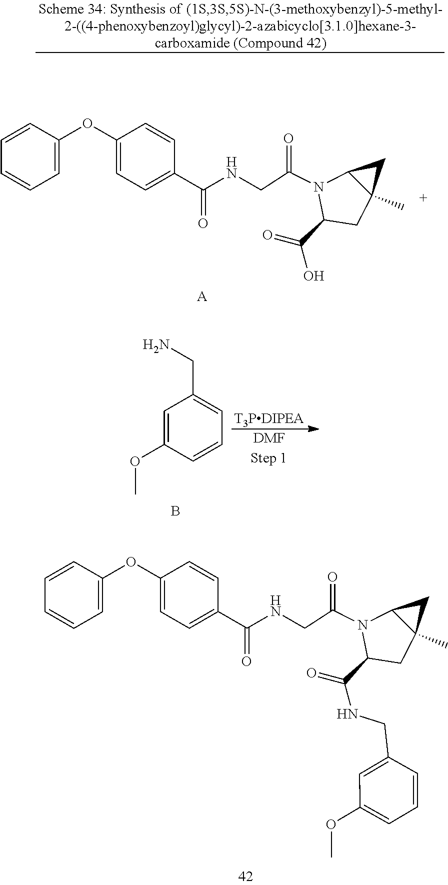

Step 1: Synthesis of (1S,3S,5S)-N-(3-methoxybenzyl)-5-methyl-2-((4-phenoxybenzoyl)-glycyl)-2-azabicyclo[3.1.0]hexane-3-carboxamide (Compound 42). To a mixture of (1 S,3S,5S)-5-methyl-2-((4-phenoxybenzoyl)glycyl)-2-azabicyclo[3.1.0]hexane-3-carboxylic acid (A; 30 mg, 0.076 mmol) and (3-methoxyphenyl)methanamine (B; 16 mg, 0.114 mmol) in DMF (1 mL) was added DIPEA (40 mg, 0.304 mmol) and T₃P (42 mg, 0.152 mmol) at room temperature under N2 atmosphere and the mixture was stirred at room temperature overnight. The mixture was quenched with saturated aq.NaHCO₃ solution and extracted with ethyl acetate twice. The combined organic layers were washed with water and brine, dried over anhydrous Na₂SO₄, filtered and concentrated to dryness. The residue was purified by prep-HPLC to give Compound 42 (18.5 mg, 47% yield) as a white solid. ¹H NMR (400 MHz, CD₃OD) δ 7.87-7.78 (m, 2H), 7.41 (t, J=8.0 Hz, 2H), 7.18 (dd, J=20.2, 7.7 Hz, 2H), 7.06 (d, J=8.3 Hz, 2H), 6.98 (d, J=8.8 Hz, 2H), 6.85 (d, J=2.4 Hz, 2H), 6.75 (dd, J=8.1, 2.2 Hz, 1H), 4.83 (d, J=3.4 Hz, 1H), 4.38 (d, J=16.0 Hz, 2H), 4.31 (d, J=9.4 Hz, 2H), 3.72 (s, 3H), 3.49-3.35 (m, 1H), 2.41 (t, J=12.4 Hz, 1H), 2.16 (dd, J=13.3, 3.4 Hz, 1H), 1.29 (s, 3H), 1.17 (dd, J=5.7, 2.4 Hz, 1H), 0.77 (t, J=5.6 Hz, 1H); LC/MS (ESI) m/z: 514 (M+H)⁺.

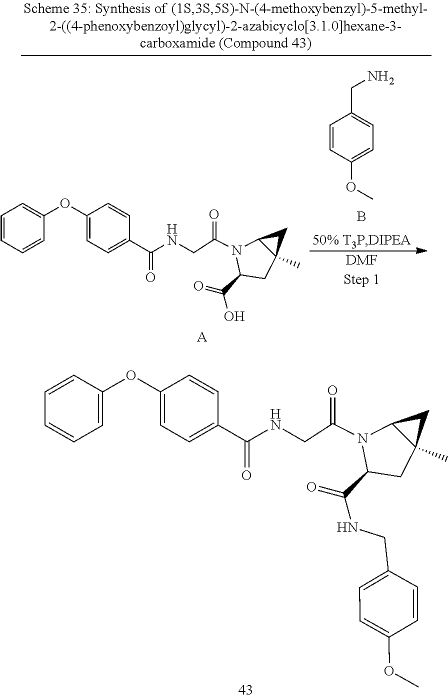

Step 1: Synthesis of (1S,3S,5S)-N-(4-methoxybenzyl)-5-methyl-2-((4-phenoxy-benzoyl)-glycyl)-2-azabicyclo[3.1.0]hexane-3-carboxamide (Compound 43). To a mixture of compound A (30 mg, 0.076 mmol) and compound B (21 mg, 0.152 mmol) in DMF (1 mL) was added DIPEA (30 mg, 0.228 mmol) and T₃P (72 mg, 0.114 mmol, 50% wt in ethyl acetate) at room temperature under N₂ atmosphere, and the mixture was stirred at room temperature overnight. The mixture was quenched with saturated aq. NaHCO₃ solution and extracted with ethyl acetate twice. The combined organic layers were washed with water and brine, dried over anhydrous Na₂SO₄, filtered, and concentrated to dryness under reduced pressure. The residue was purified by prep-HPLC to give Compound 43 (5.9 mg, 15% yield) as a white solid. ¹H NMR (400 MHz, CD₃OD) δ 7.84-7.78 (m, 2H), 7.44-7.39 (m, 2H), 7.20 (dd, J=10.3, 8.1 Hz, 3H), 7.08-7.05 (m, 2H), 7.00-6.96 (m, 2H), 6.83 (dd, J=6.7, 4.8 Hz, 2H), 4.81 (d, J=3.3 Hz, 1H), 4.38 (d, J=16.5 Hz, 1H), 4.33-4.23 (m, 3H), 3.74 (d, J=9.5 Hz, 3H), 3.37 (dd, J=6.0, 2.4 Hz, 1H), 2.38 (d, J=11.7 Hz, 1H), 2.16 (dd, J=13.3, 3.3 Hz, 1H), 1.27 (d, J=13.3 Hz, 3H), 1.14 (dd, J=5.7, 2.4 Hz, 1H), 0.75 (t, J=5.4 Hz, 1H); LC/MS (ESI) m/z: 514 (M+H)⁺.

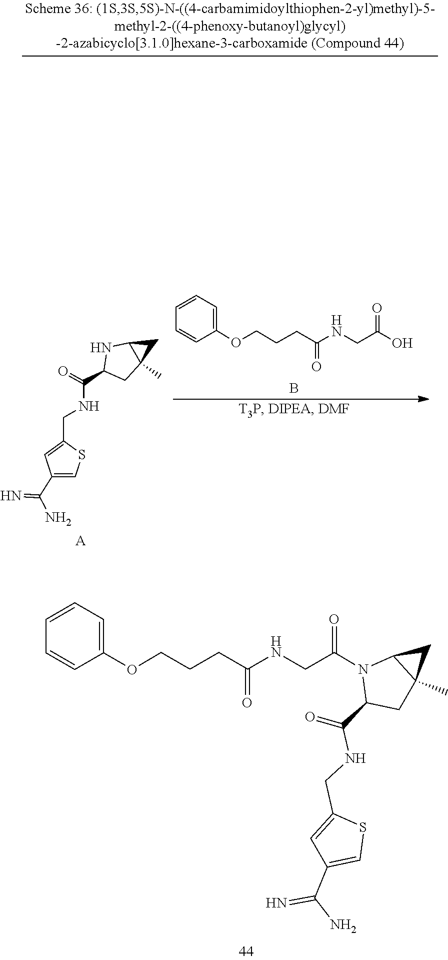

Step 1: (1S,3S,5S)-N-((4-carbamimidoylthiophen-2-yl)methyl)-5-methyl-2-((4-phenoxy-butanoyl)glycyl)-2-azabicyclo[3.1.0]hexane-3-carboxamide (Compound 44). To a mixture of compound A (50 mg, 0.2 mmol) and compound B (51 mg, 0.2 mmol) in DMF (2 mL) was added DIPEA (139 mg, 1.1 mmol) and T₃P (343 mg, 0.54 mmol, 50% wt. in EtOAc) at 0° C. under N2 atmosphere, and the mixture was stirred at 35° C. for 8 hours. The mixture was diluted with water and extracted with CHCl₃/i-PrOH (v/v=3/1) twice. The combined organic layers were dried over anhydrous Na₂SO₄, filtered, and concentrated to dryness under reduced pressure. The residue was purified by prep-HPLC to give Compound 44 (12.7 mg, 14.2% yield) as a white solid. ¹H NMR (400 MHz, CD₃OD) δ 8.55 (s, 1H), 8.21 (d, J=1.6 Hz, 1H), 7.40 (s, 1H), 7.26-7.21 (m, 2H), 6.91-6.86 (m, 3H), 4.79 (dd, J=11.6, 12.0 Hz, 1H), 4.51 (dd, J=22.4, 26.4 Hz, 2H), 4.16 (dd, J=32.4, 32.0 Hz, 2H), 3.99 (t, J=6.4 Hz, 2H), 3.35-3.32 (m, 1H), 2.47 (t, J=7.4 Hz, 2H), 2.42-2.34 (m, 1H), 2.15 (dd, J=13.6, 13.6 Hz, 1H), 2.09-2.02 (m, 2H), 1.27 (s, 3H), 1.12 (dd, J=6.0, 5.6 Hz, 1H), 0.79 (t, J=5.8 Hz, 1H); LC/MS (ESI) m/z: 498 (M+H)⁺.

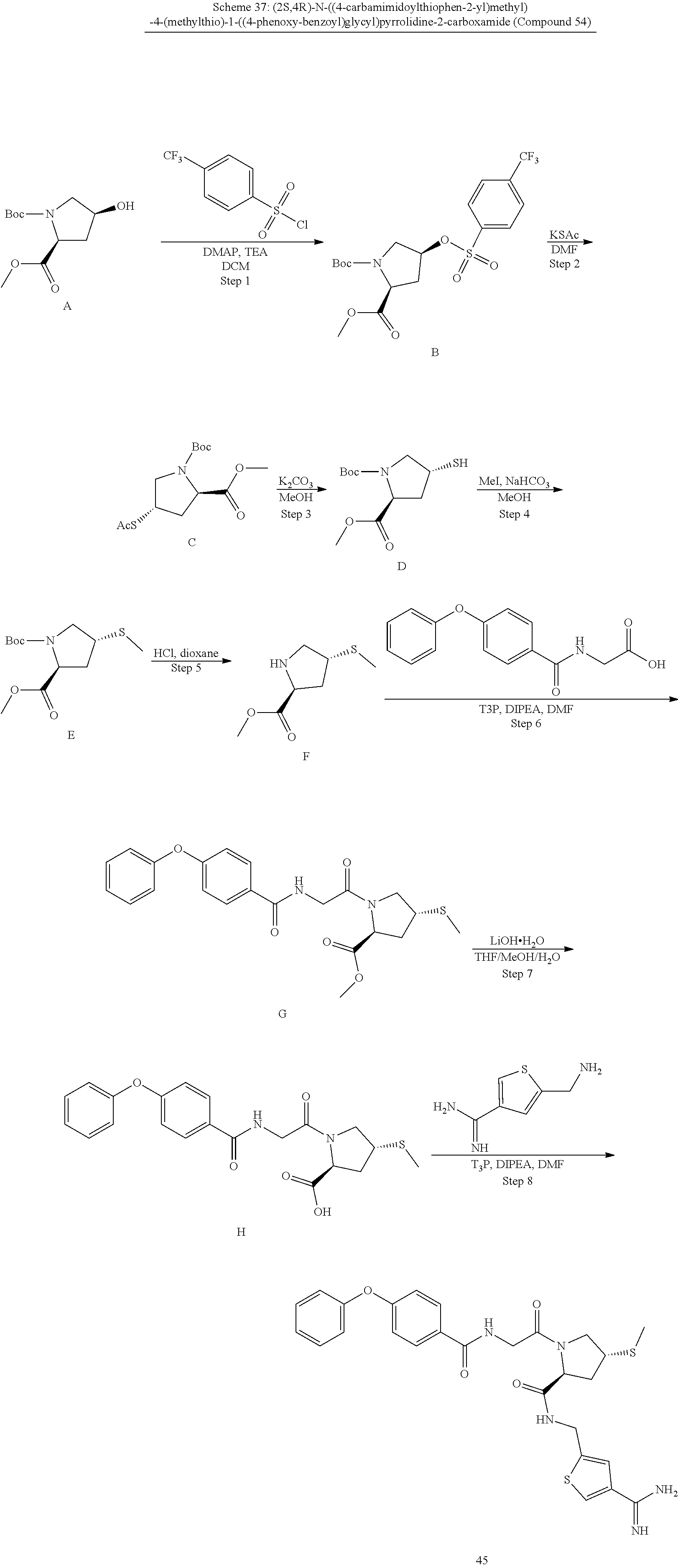

Step 1: 1-(tert-butyl) 2-methyl (2S,4S)-4-(((4-(trifluoromethyl)phenyl)-sulfonyl)oxy)-pyrrolidine-1,2-dicarboxylate (B). To a mixture of compound 1 (1 g, 4.1 mmol) and 4-(trifluoromethyl) benzenesulfonyl chloride (1.3 g, 5.3 mmol) in DCM (10 mL) was added TEA (494 mg, 4.9 mmol) and DMAP (50 mg, 0.4 mmol) at 0° C. under N₂ atmosphere. The resulting mixture was stirred at room temperature for 16 hours and then quenched with 1 N aq. HCl solution at 0° C. The mixture was extracted with EtOAc three times and the combined organic layers were washed with brine, dried over anhydrous Na₂SO₄, filtered, and concentrated to dryness under reduced pressure. The residue was purified by column chromatography on silica gel (PE:EtOAc=3:1) to give compound B (1.6 g, 86.5% yield) as a white solid. LC/MS (ESI) m/z: 354 (M+H−Boc)⁺.

Step 2: 1-(tert-butyl) 2-methyl (2S,4R)-4-(acetylthio)pyrrolidine-1,2-dicarboxylate (C). To a solution of compound B (1.6 g, 3.5 mmol) in DMF (10 mL) was added KSAc (806 mg, 7.1 mmol) at 25° C. under N₂ atmosphere. The reaction was stirred at 40° C. for 16 hours and then quenched with 1 N aq. HCl at 0° C. The mixture was extracted with EtOAc three times, and the combined organic layers were washed with brine, dried over anhydrous Na₂SO₄, filtered, and concentrated to dryness under reduced pressure. The residue was purified by column chromatography on silica gel (PE:EtOAc=5:1) to give compound C (643 mg, 60.1% yield) as a yellow oil; LC/MS (ESI) m/z: 204 (M+H−Boc)⁺.

Step 3: 1-(tert-butyl) 2-methyl (2S,4R)-4-mercaptopyrrolidine-1,2-dicarboxylate (D). To a solution of compound C (643 mg, 2.1 mmol) in MeOH (5 mL) and DCM (0.5 mL) was added K₂CO₃ (293 mg, 2.1 mmol) at 0° C. The resulting mixture was stirred at 0° C. for 2 hours, and the solvent was evaporated under reduced pressure. The residue was diluted with water and extracted with EtOAc twice. The combined organic layers were washed with brine, dried over anhydrous Na₂SO₄, filtered and concentrated to dryness under reduced pressure to give compound D (538 mg, 97.1% yield) as a yellow oil, which was used directly in next step without further purification. LC/MS (ESI) m/z: 162 (M+H−Boc)⁺.

Step 4: 1-(tert-butyl) 2-methyl (2S,4R)-4-(methylthio)pyrrolidine-1,2-dicarboxylate (E). To a solution of compound D (538 mg, 2.1 mmol) in MeOH (5 mL) was added Mel (3 g, 21.2 mmol) and NaHCO₃ (191 mg, 2.3 mmol) at 25° C. under N2 atmosphere and the reaction was stirred at 25° C. for 16 hours. The mixture was diluted with water and extracted with EtOAc twice. The combined organic layers were washed with brine, dried over anhydrous Na₂SO₄, filtered, and concentrated to dryness under reduced pressure. The residue was purified by column chromatography on silica gel (PE:EtOAc=3:1) to give compound E (314 mg, 55.3% yield) as a yellow oil. LC/MS (ESI) m/z: 176 (M+H−Boc)⁺.

Step 5: methyl (2S,4R)-4-(methylthio) pyrrolidine-2-carboxylate (F). A solution of compound E (314 mg, 1.1 mmol) in HCl/1, 4-dioxane (3 mL) was stirred at room temperature for 2 hours. The mixture was concentrated to dryness under reduced pressure and dried under vacuum to give compound F (199 mg, 99.6% yield) as a white solid, which was used directly in the next step without further purification. LC/MS (ESI) m/z: 176 (M+H)⁺.

Step 6: methyl (2R,4S)-4-(methylthio)-1-((4-phenoxybenzoyl)glycyl)pyrrolidine-2-carboxylate (G). To a mixture of compound F (199 mg, 1.1 mmol) and [(4-phenoxyphenyl)formamido]-acetic acid (308 mg, 1.1 mmol) in DMF (3 mL) was added DIPEA (881 mg, 6.8 mmol) and T₃P (2.2 g, 3.4 mmol, 50% wt in EtOAc) at 0° C. under N2 atmosphere, and the mixture was stirred at 30° C. for 16 hours. The mixture was diluted with water and extracted with EtOAc twice. The combined organic layers were washed with brine, dried over anhydrous Na₂SO₄, filtered, and concentrated to dryness under reduced pressure. The residue was purified by column chromatography on silica gel (DCM:MeOH=20:1) to give compound G (285 mg, 58.5% yield) as a yellow oil. LC/MS (ESI) m/z: 429 (M+H)⁺.

Step 7: methyl (2R,4S)-4-(methylthio)-1-((4-phenoxybenzoyl) glycyl) pyrrolidine-2-carboxylate (H). To a solution of compound G (185 mg, 0.4 mmol) in MeOH (2 mL) and THE (1 mL) was added a solution of LiOH·H₂O (18 mg, 0.4 mmol) in H₂O (1 mL) at 0° C., and the mixture was stirred at 25° C. for 5 hours. The mixture was diluted with water and extracted with EtOAc twice. The organic layers were discarded and the water layer was acidified with 0.5 M aq. HCl. The aqueous layer was extracted with EtOAc twice, and the combined organic layers were dried over anhydrous Na₂SO₄, filtered, and concentrated to dryness under reduced pressure to give compound H (148 mg, 82.7% yield) as a white solid, which was used directly in the next step without further purification. LC/MS (ESI) m/z: 415 (M+H)⁺.

Step 8: (2S,4R)-N-((4-carbamimidoylthiophen-2-yl) methyl)-4-(methylthio)-1-((4-phenoxy-benzoyl)glycyl)pyrrolidine-2-carboxamide (Compound 45). To a mixture of compound H (148 mg, 0.3 mmol) and 5-(aminomethyl)thiophene-3-carboximidamide (67 mg, 0.4 mmol) in DMF (3 mL) was added DIPEA (277 mg, 2.1 mmol) and T₃P (681 mg, 1.1 mmol, 50% wt in EtOAc) at 0° C. under N2 atmosphere, and the mixture was stirred at 35° C. for 4 hours. The mixture was diluted with water and extracted with CHCl₃/i-PrOH (v/v=3/1) three times. The combined organic layers were dried over anhydrous Na₂SO₄, filtered, and concentrated to dryness under reduced pressure. The residue was purified by prep-HPLC to give Compound 45 (5.2 mg, 2.64% yield) as a white solid. ¹H NMR (400 MHz, CD₃OD) δ 8.23 (dd, J=17.2, 17.2 Hz, 1H), 7.86-7.82 (m, 2H), 7.41 (t, J=8.0 Hz, 3H), 7.20 (t, J=7.4 Hz, 1H), 7.07-7.04 (m, 2H), 7.01-6.98 (m, 2H), 4.71-4.59 (m, 1H), 4.58 (s, 2H), 4.20 (dd, J=28.8, 29.6 Hz, 2H), 4.12-4.04 (m, 1H), 3.64-3.48 (m, 2H), 2.38-2.20 (m, 2H), 2.18 (s, 3H); LC/MS (ESI) m/z: 552 (M+H)⁺.

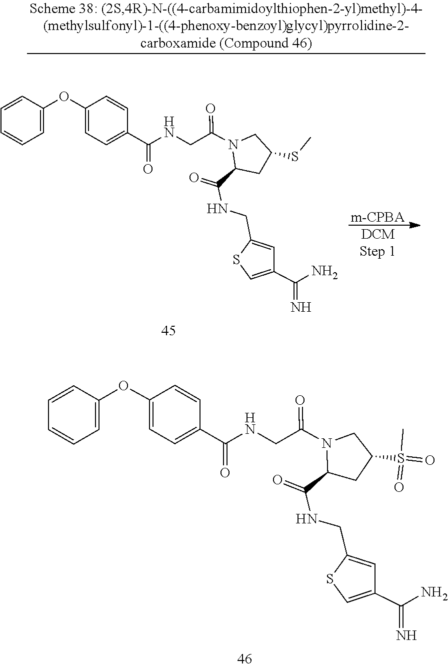 [SMILES: CS[C@@H]1C[C@@H](C(=O)NCc2cc(C(=N)N)cs2)N(C(=O)CNC(=O)c2ccc(Oc3ccccc3)cc2)C1 ; CS(=O)(=O)[C@@H]1C[C@@H](C(=O)NCc2cc(C(=N)N)cs2)N(C(=O)CNC(=O)c2ccc(Oc3ccccc3)cc2)C1]

Step 1: (2S,4R)-N-((4-carbamimidoylthiophen-2-yl)methyl)-4-(methylsulfonyl)-1-((4-phenoxybenzoyl)glycyl)pyrrolidine-2-carboxamide (Compound 46). To a mixture of Compound 45 (139 mg, 0.3 mmol) in DCM (2 mL) was added m-CPBA (131 mg, 0.8 mmol) at 0° C. under N2 atmosphere, and the mixture was stirred at 25° C. for 4 hours. The mixture was diluted with aq.Na₂CO₃ solution at 0° C. and extracted with CHCl₃/i-PrOH (v/v=3/1) three times. The combined organic layers were dried over anhydrous Na₂SO₄, filtered, and concentrated to dryness under reduced pressure. The residue was purified by prep-HPLC to give Compound 46 (10.1 mg, 6.8% yield) as a white solid. ¹H NMR (400 MHz, CD₃OD) δ 8.23 (dd, J=16.8, 16.8 Hz, 1H), 7.86-7.81 (m, 2H), 7.45-7.37 (m, 3H), 7.20 (t, J=7.6 Hz, 1H), 7.06 (dd, J=8.8, 8.4 Hz, 2H), 7.03-6.97 (m, 2H), 4.70 (dd, J=8.8, 8.8 Hz, 1H), 4.65-4.53 (m, 2H), 4.33-4.18 (m, 2H), 4.18-4.12 (m, 2H), 4.11-3.91 (m, 1H), 3.07 (s, 3H), 2.90-2.70 (m, 1H), 2.56-2.33 (m, 1H); LC/MS (ESI) m/z: 584 (M+H)⁺.

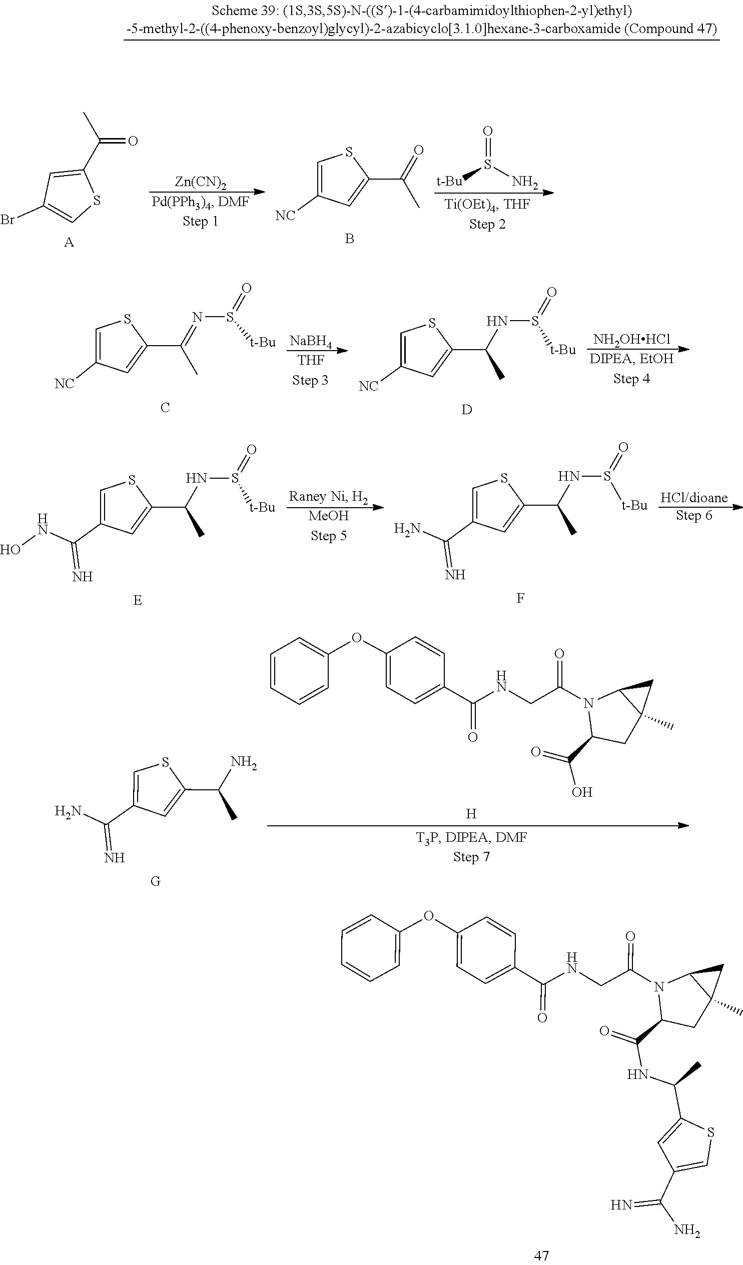

Step 1: 5-acetylthiophene-3-carbonitrile (B). To a solution of compound A (3.0 g, 14.60 mmol) in DMF (30 mL) was added Zn(CN)₂ (2.23 g, 18.98 mmol) and Pd(PPh₃)₄ (846 mg, 0.73 mmol) at 25° C. under N2 atmosphere. The mixture was stirred at 80° C. for 16 hours. The mixture was cooled to 25° C. and poured into iced-water. The mixture was extracted with EtOAC twice and the combined organic layers were washed with brine, dried over anhydrous Na₂SO₄, filtered, and concentrated to dryness under reduced pressure. The residue was purified by flash chromatography (PE:EtOAc=5:1) to give compound B (0.6 g, 27.3% yield) as a white solid. LC/MS (ESI) (m/z): 152 (M+H)⁺.

Step 2: (R,E)-N-(1-(4-cyanothiophen-2-yl)ethylidene)-2-methylpropane-2-sulfinamide (C). To a solution of (R)-2-methylpropane-2-sulfinamide (647 mg, 5.34 mmol) in THE (6 mL) was added mixture of compound B (667 mg, 4.45 mmol) in THE (6 mL) and Ti(OEt)₄ (2.84 g, 12.46 mmol) at 25° C. under N2 atmosphere. The mixture was stirred at 70° C. for 16 hours. The reaction was cooled to 25° C., poured into brine and filtered. The filter cake was washed with EtOAc twice. The filtrate was washed with brine, dried over anhydrous Na₂SO₄, filtered, and concentrated to dryness under reduced pressure. The residue was purified by flash chromatography (PE EtOAc=5:1) to give compound C (1.04 g, 92.0% yield) as a yellow solid. LC/MS (ESI) (m/z): 255 (M+H)⁺.

Step 3: (R)-N-((S*)-1-(4-cyanothiophen-2-yl)ethyl)-2-methylpropane-2-sulfinamide (D). To a solution of compound 3 (350 mg, 1.38 mmol) in THE (5 mL) was added NaBH₄ (78.5 mg, 2.07 mmol) in portions at −20° C. under N2 atmosphere and the mixture was stirred at 25° C. for 1 hour. The mixture was quenched with saturated aq.NH₄Cl solution at 0° C. and extracted with DCM twice. The combined organic layers were washed with brine, dried over anhydrous Na₂SO₄, filtered, and concentrated to dryness under reduced pressure. The residue was purified by flash chromatography (PE:EtOAc=1:1) and further purified by SFC to give compound D (90 mg, 25.6% yield) as a colorless oil. LC/MS (ESI) m/z: 257 (M+H)⁺.

Step 4: 5-((S*)-1-(((R)-tert-butylsulfinyl)amino)ethyl)-N-hydroxythiophene-3-carboximidamide (E). To a solution of compound D (90 mg, 0.35 mmol) in EtOH (5 mL) was added NH₂OH HCl (61 mg, 0.88 mmol) and DIPEA (0.17 mL, 1.05 mmol), and the mixture was stirred at 25° C. for 16 hours. The mixture was concentrated to dryness under reduced pressure and the residue was purified by flash chromatography (DCM:MeOH=10:1) to give compound E (100 mg, 98.0% yield) as a colorless oil. LC/MS (ESI) m/z: 290 (M+H)⁺.

Step 5: 5-((S*)-1-(((R)-tert-butylsulfinyl)amino)ethyl)thiophene-3-carboximidamide (F). To a solution of compound E (102 mg, 0.35 mmol) in MeOH (5 mL) was added Raney Ni (20 mg) and AcOH (0.1 mL) under N2 atmosphere. The mixture was stirred under a H₂ balloon at 25° C. for 16 hours. The mixture was filtered and filtrate was concentrated to dryness under reduced pressure to give compound F (96 mg, 99.9% yield) as a light-yellow solid, which was used directly in the next step without further purification. LC/MS (ESI) (m/z): 274 (M+H)⁺.

Step 6: (S*)-5-(1-aminoethyl)thiophene-3-carboximidamide (G). A solution of compound F (96 mg, 0.35 mmol) in HCl/1, 4-dioxane (6 mL) was stirred at room temperature for 1 hour. The reaction mixture was concentrated to dryness under reduced pressure and dried under vacuum to give compound G (50 mg, yield 83.3%) as a colorless oil, which was used directly in the next step without further purification. LC/MS (ESI) m/z: 170 (M+H)⁺.

Step 7: (1S,3S,5S)-N-((S*)-1-(4-carbamimidoylthiophen-2-yl)ethyl)-5-methyl-2-((4-phenoxy-benzoyl)glycyl)-2-azabicyclo[3.1.0]hexane-3-carboxamide (Compound 47). To a mixture of compound G (30 mg, 0.18 mmol) and compound H (70 mg, 0.18 mmol) in DMF (5 mL) was added DIPEA (0.17 mL, 1.08 mmol) and T₃P (339 mg, 0.54 mmol, 50% wt in EtOAc) at 0° C. under N2 atmosphere, and the mixture was stirred at 25° C. for 16 hours. The mixture was diluted with water and extracted with CHCl₃/i-PrOH (v/v=3/1) twice. The organic layer was dried over anhydrous Na₂SO₄, filtered and concentrated to dryness under reduced pressure. The residue was purified by flash chromatography (DCM MeOH=5:1) and further purified by prep-HPLC to give Compound 47 (3.0 mg, 3.0% yield) as a white solid. ¹H NMR (400 MHz, CD₃OD) δ 8.17 (d, J=1.6 Hz, 1H), 7.86-7.78 (m, 2H), 7.46-7.37 (m, 3H), 7.20 (t, J=7.4 Hz, 1H), 7.09-7.04 (m, 2H), 7.01-6.96 (m, 2H), 5.25 (d, J=6.9 Hz, 1H), 4.82 (d, J=3.4 Hz, 1H), 4.30 (q, J=16.2 Hz, 2H), 3.50-3.40 (m, 1H), 2.42 (t, J=12.5 Hz, 1H), 2.17 (dd, J=13.3, 3.3 Hz, 1H), 1.64 (t, J=6.5 Hz, 3H), 1.30 (s, 3H), 1.15-1.11 (m, 1H), 0.82 (t, J=5.4 Hz, 1H); LC/MS (ESI) m/z: 546 (M+H)⁺.

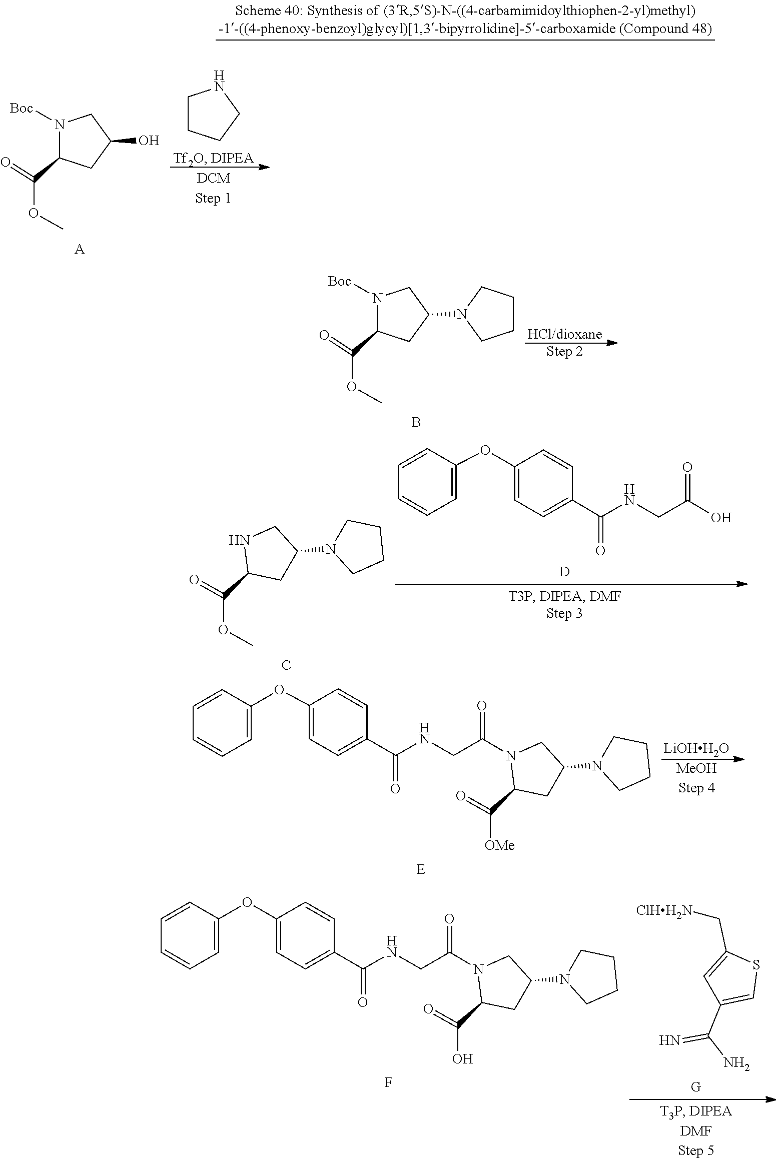

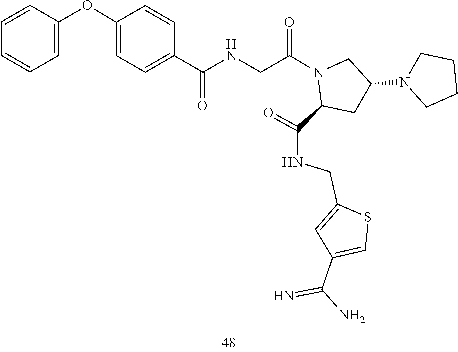 [SMILES: N=C(N)c1csc(CNC(=O)[C@@H]2C[C@@H](N3CCCC3)CN2C(=O)CNC(=O)c2ccc(Oc3ccccc3)cc2)c1]

Step 1: 1′-(tert-butyl) 5′-methyl (3′R,5'S)-[1,3′-bipyrrolidine]-1′,5′-dicarboxylate (B). To a mixture of compound A (500 mg, 2.04 mmol) and DIPEA (657 mg, 5.10 mmol) in DCM (10 mL) was added Tf₂O (863 mg, 3.06 mmol) at −20° C. After stirring at −20° C. for another 45 min, pyrrolidine (435 mg, 6.12 mmol) was added in one portion and the mixture was stirred at room temperature overnight. The mixture was quenched with 1 N aq.NaOH solution (5 mL), diluted with saturated aq.N aHCO₃ solution and extracted with DCM twice. The combined organic layers were washed with brine, dried over anhydrous Na₂SO₄, filtered, and concentrated to dryness under reduced pressure. The residue was purified by column chromatography on silica gel (DCM:MeOH=25:1 to 20:1) to give compound B (336 mg, 55.2% yield) as a yellow oil. LC/MS (ESI) m/z: 299 (M+H)⁺.

Step 2: methyl (3′R,5'S)-[1,3′-bipyrrolidine]-5′-carboxylate (C). A solution of compound B (200 mg, 0.67 mmol) in HCl/1, 4-dioxane (3 mL) was stirred at room temperature for 1 hour. The reaction mixture was concentrated to dryness under reduced pressure and dried under vacuum to give compound C (132.8 mg, 100% yield) as a yellow oil, which was used directly in the next step without further purification. LC/MS (ESI) m/z: 199 (M+H)⁺.

Step 3: methyl (3′R,5'S)-1′-((4-phenoxybenzoyl)glycyl)-[1,3′-bipyrrolidine]-5′-carboxylate (E). To a mixture of compound D (120 mg, 0.442 mmol) and compound C (132.8 mg, 0.664 mmol) in DMF (3 mL) was added DIPEA (171 mg, 1.33 mmol) and T₃P (563 mg, 0.885 mmol, 50% in ethyl acetate) at 0° C. under N2 atmosphere, and the mixture was stirred at 35° C. for 4 hours. The mixture was quenched with saturated aq. NaHCO₃ solution and extracted with ethyl acetate twice. The combined organic layers were washed with water and brine, dried over anhydrous Na₂SO₄, filtered, and concentrated to dryness under reduced pressure. The residue was purified by column chromatography on silica gel (dichloromethane:methanol=100:1 to 10:1) to give compound E (180 mg, 90.1% yield) as a light-yellow oil. LC/MS (ESI) (m/z): 452 (M+H)⁺.

Step 4: (3′R,5'S)-1′-((4-phenoxybenzoyl)glycyl)-[1,3′-bipyrrolidine]-5′-carboxylic acid (F). To a solution of compound E (180 mg, 0.399 mmol) in MeOH (2 mL) and water (0.5 mL) was added LiOH·H₂O (17 mg, 0.399 mmol) at 0° C. and the mixture was stirred at room temperature for 16 hours. The mixture was acidified with 1 N aq. HCl to pH=3 and concentrated to dryness under reduced pressure to give compound 6 (150 mg, 86.0% yield) as a light-yellow solid, which was used directly in next step without further purification. LC/MS (ESI) (m/z): 438 (M+H)⁺.

Step 5: (3′R,5'S)-N-((4-carbamimidoylthiophen-2-yl)methyl)-1′-((4-phenoxybenzoyl)glycyl)-[1,3′-bipyrrolidine]-5′-carboxamide (Compound 48). To a mixture of compound F (150 mg, 0.343 mmol) and compound G (98.6 mg, 0.514 mmol) in DMF (5 mL) was added DIPEA (132.7 mg, 1.03 mmol) and T₃P (436.1 mg, 0.686 mmol, 50% wt in ethyl acetate) at 0° C. under N2 atmosphere, and the mixture was stirred at 25° C. for 16 hours. The mixture was diluted with water and extracted with CHCl₃/i-PrOH (v/v=3/1) twice. The combined organic layers were dried over anhydrous Na₂SO₄, filtered, and concentrated to dryness under reduced pressure. The residue was purified by column chromatography on silica gel (dichloromethane:methanol=5:1 to 3:1) and further purified by prep-HPLC to give Compound 48 (20.4 mg, 10.4% yield) as a white solid. ¹H NMR (400 MHz, CD₃OD) δ 8.25 (dd, J=17.5, 1.4 Hz, 1H), 7.85 (d, J=8.6 Hz, 2H), 7.54-7.38 (m, 3H), 7.21 (t, J=7.4 Hz, 1H), 7.06 (d, J=7.7 Hz, 2H), 7.03-6.97 (m, 2H), 4.71 (dd, J=15.0, 10.0 Hz, 1H), 4.67-4.47 (m, 2H), 4.31-3.95 (m, 5H), 3.92-3.56 (m, 2H), 3.52-3.33 (m, 1H), 3.29-3.04 (m, 1H), 2.73-2.43 (m, 2H), 2.13 (s, 4H); LC/MS (ESI) m/z: 575 (M+H)⁺.

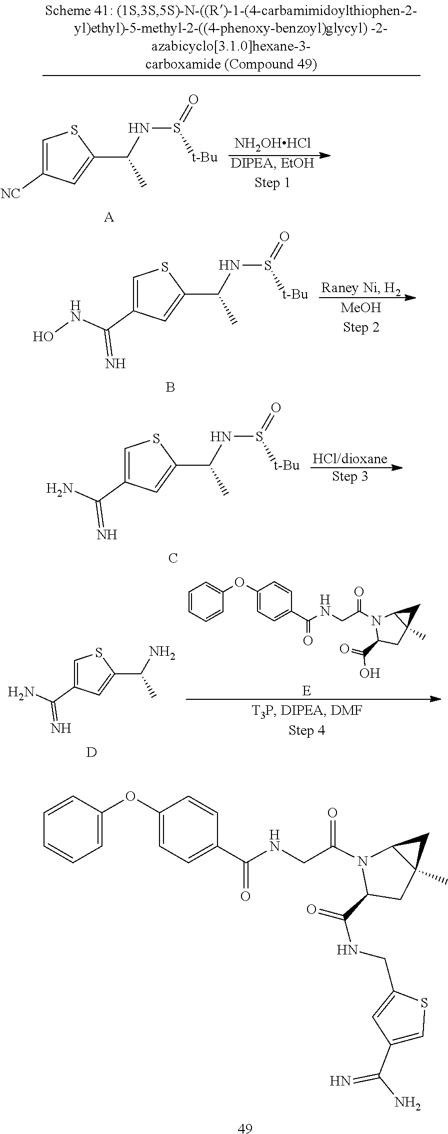

Step 1: 5-((1R*)-1-((tert-butDylsulfinyl)amino)ethyl)-N-hydroxythiophene-3-carboximidamide (B). To a solution of compound A (120 mg, 0.47 mmol) in EtOH (2.0 mL) was added NH₂OH HCl (81 mg, 1.17 mmol) and DIPEA (0.23 mL, 1.41 mmol), and the mixture was stirred at 25° C. for 16 hours. The residue was purified by flash chromatography (DCM:MeOH=10:1) to give compound B (130 mg, 96.0% yield) as a colorless oil. LC/MS (ESI) m/z: 290 (M+H)⁺.

Step 2: 5-((1R*)-1-((tert-butylsulfinyl)amino)ethyl)thiophene-3-carboximidamide (C). To a solution of compound B (130 mg, 0.45 mmol) in MeOH (4 mL) was added Raney Ni (25 mg) and AcOH (0.1 mL) under N2 atmosphere, and the mixture was stirred at 25° C. under H₂ atmosphere for 16 hours. The mixture was filtered and filtrate was concentrated to dryness under reduced pressure to give compound C (120 mg, 98% yield) as a light-yellow solid. The crude product was used directly in next step without further purification. LC/MS (ESI) (m/z): 274(M+H)⁺.

Step 3: (R*)-5-(1-aminoethyl)thiophene-3-carboximidamide (D). A solution of compound C (120 mg, 0.43 mmol) in HCl/1, 4-dioxane (6 mL) was stirred at room temperature for 1 hour. The reaction mixture was concentrated to dryness under reduced pressure and dried under vacuum to give compound D (70 mg, 92% yield) as a colorless oil, which was used directly in the next step without further purification. LC/MS (ESI) m/z: 170 (M+H)⁺.

Step 4: (1S,3S,5S)-N-((R*)-1-(4-carbamimidoylthiophen-2-yl)ethyl)-5-methyl-2-((4-phenoxy-benzoyl)glycyl)-2-azabicyclo[3.1.0]hexane-3-carboxamide (Compound 49). To a mixture of compound D (40 mg, 0.23 mmol) and compound E (40 mg, 0.10 mmol) in DMF (2 mL) was added DIPEA (0.23 mL, 2.80 mmol) and T₃P (191 mg, 0.30 mmol, 50% wt in EtOAc) at 0° C. under N2 atmosphere and the mixture was stirred at 25° C. for 16 hours. The mixture was diluted with water and extracted with CHCl₃/i-PrOH (v/v=3/1) twice. The combined organic layers were dried over anhydrous Na₂SO₄, filtered and concentrated to dryness under reduced pressure. The residue was purified by flash chromatography (DCM:MeOH=5:1) and further purified by prep-HPLC to give Compound 49 (6.0 mg, 10% yield) as white solid. ¹H NMR (400 MHz, DMSO-d₆) δ 9.12 (s, 2H), 9.12 (s, 2H), 8.86-8.56 (m, 3H), 8.91-8.51 (m, 3H), 8.39 (d, J=7.6 Hz, 1H), 8.35 (dd, J=25.5, 7.8 Hz, 2H), 8.32 (d, J=8.0 Hz, 1H), 7.88 (t, J=7.2 Hz, 2H), 7.88 (t, J=7.2 Hz, 2H), 7.55 (d, J=6.9 Hz, 1H), 7.49-7.41 (m, 2H), 7.23 (t, J=7.4 Hz, 1H), 7.12-7.07 (m, 2H), 7.06-7.01 (m, 2H), 5.10 (t, J=7.0 Hz, 1H), 4.67 (dd, J=11.4, 2.9 Hz, 1H), 4.30 (dd, J=16.3, 5.7 Hz, 1H), 4.04 (dd, J=16.3, 5.5 Hz, 1H), 3.51 (d, J=5.1 Hz, 1H), 2.37-2.25 (m, 1H), 2.02 (dd, J=13.3, 2.9 Hz, 1H), 1.49 (dd, J=24.4, 6.9 Hz, 3H), 1.23 (s, 3H), 1.11 (d, J=2.9 Hz, 1H); LC/MS (ESI) m/z: 546 (M+H)⁺.

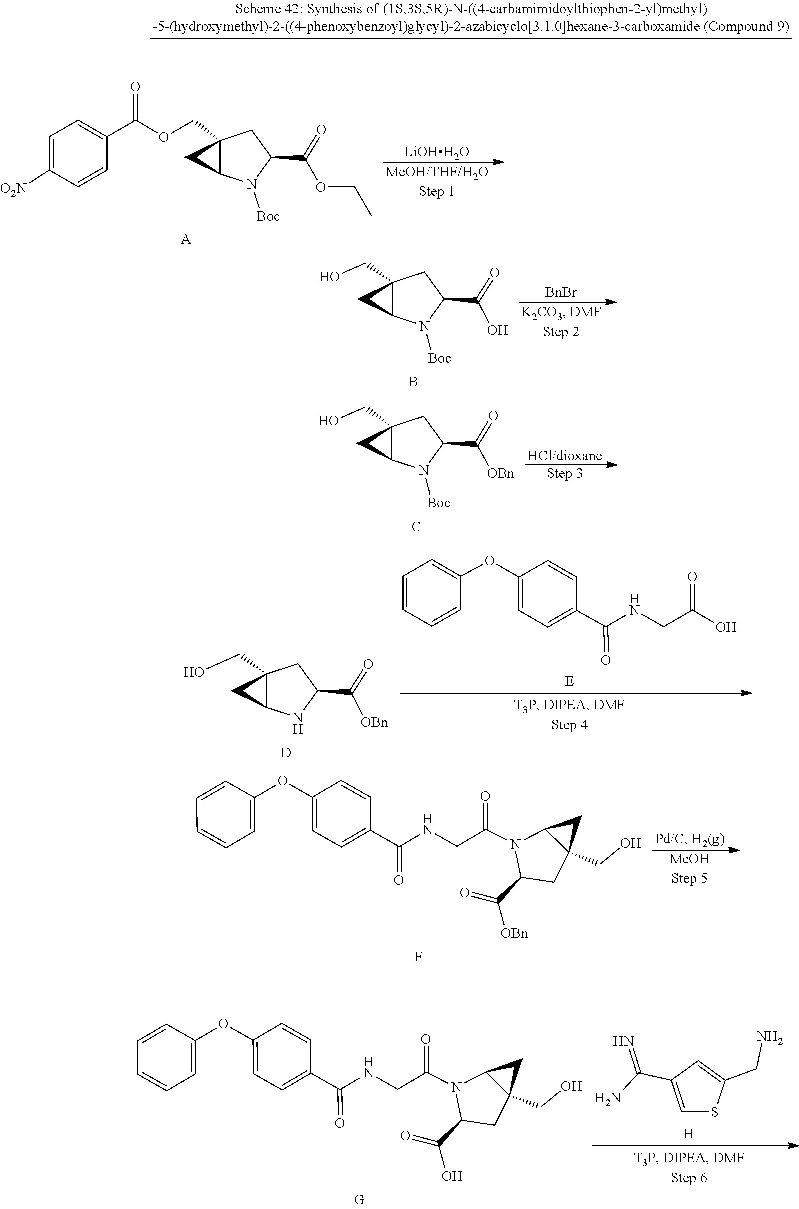

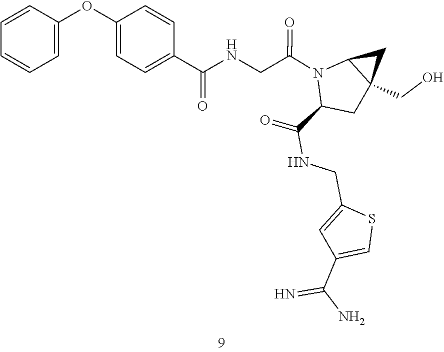 [SMILES: N=C(N)c1csc(CNC(=O)[C@@H]2C[C@]3(CO)C[C@@H]3N2C(=O)CNC(=O)c2ccc(Oc3ccccc3)cc2)c1]

Step 1: (1S,3S,5R)-2-(tert-butoxycarbonyl)-5-(hydroxymethyl)-2-azabicyclo[3.1.0]hexane-3-carboxylic acid (B). To a solution of compound A (600 mg, 1.4 mmol) in MeOH (4 mL) and THE (2 mL) was added a solution of LiOH·H₂O (174 mg, 4.2 mmol) in water (2 mL) at 0° C. and the mixture was stirred at room temperature for 16 hours. The mixture was washed with EtOAc twice and the aqueous layer was acidified with 1 N aq. HCl to pH˜3 and extracted with EtOAc three times. The combined organic layers were dried over anhydrous Na₂SO₄, filtered, and concentrated to dryness under reduced pressure to give compound B (350 mg, 98.9% yield) as a white solid, which was used directly in next step without further purification. LC/MS (ESI) (m/z): 258 (M+H)⁺.

Step 2: 3-benzyl 2-(tert-butyl) (1S,3S,5R)-5-(hydroxymethyl)-2-azabicyclo[3.1.0]hexane-2,3-dicarboxylate (C). To a solution of compound B (350 mg, 1.40 mmol) in DMF (10 mL) was added K₂CO₃ (376 mg, 2.80 mmol) and BnBr (349 mg, 2.10 mmol) at 0° C. under N2 atmosphere and the mixture was stirred at 25° C. for 3 hours. The mixture was diluted with EtOAc and washed with saturated aq.NH₄Cl solution and brine, dried over anhydrous Na₂SO₄, filtered, and concentrated to dryness under reduced pressure. The residue was purified by chromatography on silica gel (PE:EtOAc=1:1) to give compound C (425 mg, 90.0% yield) as a colorless oil. LC/MS (ESI) m/z: 348 (M+H)⁺.

Step 3: benzyl (1S,3S,5R)-5-(hydroxymethyl)-2-azabicyclo[3.1.0]hexane-3-carboxylate (D). A solution of compound C (425 mg, 1.22 mmol) in HCl/1,4-dioxane (6 mL) was stirred at room temperature for 1 hour. The reaction mixture was concentrated to dryness under reduced pressure and dried under vacuum to give compound D (302 mg, 100% yield) as a colorless oil, which was used directly in the next step without further purification. LC/MS (ESI) m/z: 248 (M+H)⁺.

Step 4: benzyl (1S,3S,5R)-5-(hydroxymethyl)-2-((4-phenoxybenzoyl)glycyl)-2-azabicyclo-[3.1.0]hexane-3-carboxylate (F). To a mixture of compound D (150 mg, 0.61 mmol) and compound E (165 mg, 0.61 mmol) in DMF (5 mL) was added DIPEA (0.60 mL, 3.66 mmol) and T₃P (1.16 g, 1.83 mmol, 50% wt in EtOAc) at 0° C. under N2 atmosphere, and the mixture was stirred at 25° C. for 16 hours. The mixture was diluted with water and extracted with EtOAc twice. The combined organic layers were dried over anhydrous Na₂SO₄, filtered, and concentrated to dryness under reduced pressure. The residue was purified by column chromatography on silica gel (DCM:MeOH=97:3) to give compound F (276 mg, 90.8% yield) as a yellow solid. LC/MS (ESI) m/z: 501 (M+H)⁺.

Step 5: (1S,3S,5R)-5-(hydroxymethyl)-2-((4-phenoxybenzoyl)glycyl)-2-azabicyclo[3.1.0]-hexane-3-carboxylic acid (G). To a solution of compound F (276 mg, 0.55 mmol) in MeOH (5 mL) was added Pd/C (20 mg) at 25° C. under N2 atmosphere, and the mixture was stirred under a H₂ balloon at 25° C. for 1 hour. The mixture was filtered, and the filtrate was concentrated to dryness under reduced pressure to give compound G (200 mg, 88.5% yield) as a white solid, which was used directly in the next step without further purification. LC/MS (ESI) (m/z): 411 (M+H)⁺.

Step 6: (1S,3S,5R)-N-((4-carbamimidoylthiophen-2-yl)methyl)-5-(hydroxymethyl)-2-((4-phenoxybenzoyl)glycyl)-2-azabicyclo[3.1.0]hexane-3-carboxamide (Compound 9). To a mixture of compound G (120 mg, 0.29 mmol) and compound H (68 mg, 0.44 mmol) in DMF (5 mL) was added DIPEA (0.29 mL, 1.74 mmol) and T₃P (558 mg, 0.87 mmol, 50% wt in EtOAc) at 0° C. under N2 atmosphere, and the mixture was stirred at 25° C. for 16 hours. The mixture was diluted with water and extracted with CHCl₃/i-PrOH (v/v=3/1) twice. The combined organic layers were dried over anhydrous Na₂SO₄, filtered, and concentrated to dryness under reduced pressure. The residue was purified by column chromatography on silica gel (DCM:MeOH=5:1) and further purified by prep-HPLC to give Compound 9 (2.0 mg, 17.9% yield) as a white solid. ¹H NMR (400 MHz, CD₃OD) δ 8.53 (s, 1H), 8.21 (s, 1H), 7.84 (d, J=8.7 Hz, 2H), 7.41 (t, J=7.9 Hz, 3H), 7.20 (t, J=7.4 Hz, 1H), 7.06 (d, J=7.9 Hz, 2H), 7.00 (d, J=8.5 Hz, 2H), 4.88 (s, 1H), 4.61-4.49 (m, 2H), 4.40-4.27 (m, 2H), 3.66 (d, J=11.7 Hz, 1H), 3.57-3.46 (m, 2H), 2.68 (t, J=12.5 Hz, 1H), 2.13 (dd, J=13.3, 3.4 Hz, 1H), 1.22 (dd, J=5.8, 2.4 Hz, 1H), 1.00 (t, J=5.7 Hz, 1H); LC/MS (ESI) m/z: 548 (M+H)⁺.

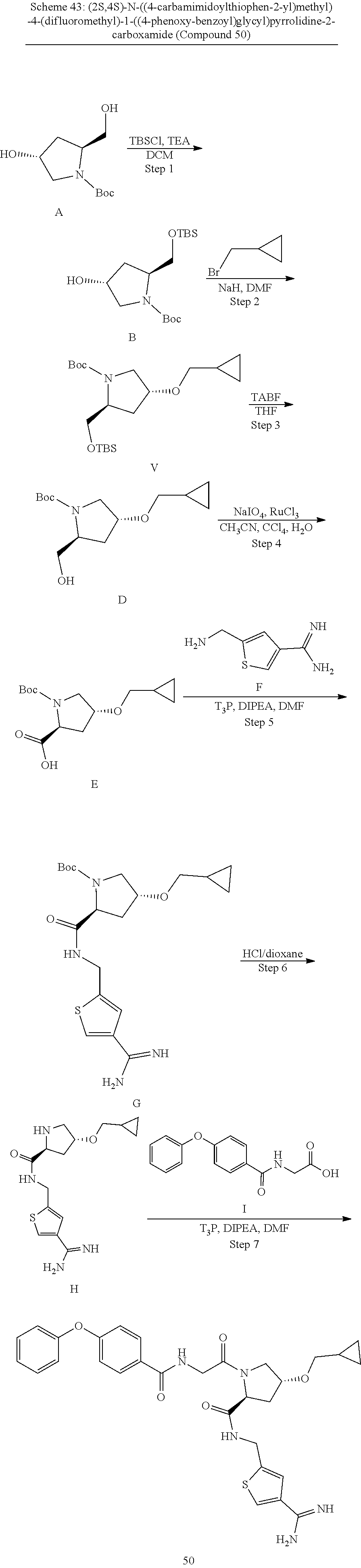

Step 1: tert-butyl (2S,4R)-2-(((tert-butyldimethylsilyl)oxy)methyl)-4-hydroxypyrrolidine-1-carboxylate (B). To a solution of compound A (1 g, 4.61 mmol) in DCM (10 mL) was added TEA (0.6 mL, 4.61 mmol) and TBSCl (694 mg, 4.61 mmol) at 0° C. under N2 atmosphere. The reaction was stirred at 25° C. for 16 hours and quenched with water. The mixture was extracted with EtOAc twice and the combined organic layers were washed with brine, dried over anhydrous Na₂SO₄, filtered, and concentrated to dryness under reduced pressure. The residue was purified by flash chromatography on silica gel (PE:EtOAc=3:1) to give compound B (1.1 g, yield 72.1%) as an oil. LC/MS (ESI) m/z: 232 (M+H−Boc)⁺.

Step 2: tert-butyl (2S,4R)-2-(((tert-butyldimethylsilyl)oxy)methyl)-4-(cyclopropylmethoxy) pyrrolidine-1-carboxylate (C). To a solution of compound B (1 g, 3.02 mmol) in DMF (8 mL) was added NaH (519 mg 12.9 mmol, 60% weight oil dispersion) at 0° C. under N₂ atmosphere and the mixture was stirred at room temperature for 30 min. (Bromomethyl)cyclopropane (1.4 g, 10.5 mmol) was added to the mixture at 0° C. and the reaction was stirred at 25° C. for 7 hours. The reaction was quenched with water at 0° C. and extracted with EtOAc twice. The combined organic layers were washed with brine, dried over anhydrous Na₂SO₄, filtered, and concentrated to dryness under reduced pressure. The residue was purified by flash chromatography on silica gel (PE:EtOAc=3:1) to give compound C (651 mg, yield 55.9%) as an oil. LC/MS (ESI) m/z: 286 (M+H−Boc)⁺.

Step 3: tert-butyl (2S,4R)-4-(cyclopropylmethoxy)-2-(hydroxymethyl)pyrrolidine-1-carboxylate (D). To a solution of compound C (651 mg, 1.69 mmol) in THE (5 mL) was added TBAF (1.1 g, 3.38 mmol) at 0° C. and the reaction was stirred at 25° C. for 1.5 hours. The mixture was diluted with EtOAc and washed with saturated aq. NH₄Cl. The aqueous layer was extracted with EtOAc twice and the combined organic layers were washed with brine, dried over anhydrous Na₂SO₄, filtered, and concentrated to dryness under reduce pressure. The residue was purified by flash chromatography on silica gel (PE:EtOAc=3:1) to give compound D (369 mg, yield 53.2%) as an oil. LC/MS (ESI) m/z: 172 (M+H−Boc)⁺.

Step 4: (2S,4R)-1-(tert-butoxycarbonyl)-4-(cyclopropylmethoxy)pyrrolidine-2-carboxylic acid (E). To a solution of compound D (320 mg, 1.18 mmol) in MeCN (2 mL), CCl₄ (2 mL), and H₂O (3 mL) was added NaIO₄ (1.0 g, 4.72 mmol) at 25° C. and the mixture was stirred at 25° C. for 0.5 hour. RuCl₃ (6 mg, 0.02 mmol) was added and the reaction was stirred at 25° C. for another 2 hours. The precipitate was filtered and the solid was washed with DCM. The filtrate was separated and the aqueous layer was extracted with DCM twice. The combined organic layers were dried over anhydrous Na₂SO₄, filtered, and concentrated to dryness under reduced pressure. The residue was purified by flash chromatography on silica gel (DCM:MeOH=10:1) to give compound E (113 mg, yield 33.6%) as a yellow oil. LC/MS (ESI) m/z: 186 (M+H−Boc)⁺.

Step 5: tert-butyl (2S,4R)-2-(((4-carbamimidoylthiophen-2-yl)methyl)carbamoyl)-4-(cyclopropylmethoxy)pyrrolidine-1-carboxylate (G). To a mixture of compound E (113 mg, 0.39 mmol) and F (73 mg, 0.47 mmol) in DMF (2 mL) was added DIPEA (0.4 mL, 2.34 mmol) and T₃P (744 mg, 1.17 mmol, 50% wt in EtOAc) at 0° C. under N2 atmosphere and the mixture was stirred at 35° C. for 5 hours. The mixture was diluted with water and extracted with CHCl₃/i-PrOH (v/v=3/1) three times. The combined organic layers were washed with saturated aq. NaHCO₃, dried over anhydrous Na₂SO₄, filtered, and concentrated to dryness under reduced pressure. The residue was purified by flash chromatography on silica gel (DCM:MeOH=10:1) to give compound G (88 mg, yield 53.6%) as a yellow oil. LC/MS (ESI) m/z: 322 (M+H−Boc)⁺.

Step 6: (2S,4R)-N-((4-carbamimidoylthiophen-2-yl)methyl)-4-(cyclopropylmethoxy) pyrrolidine-2-carboxamide (H). A mixture of compound G (88 mg, 0.21 mmol) in HCl/1,4-dioxane (2 mL) was stirred at room temperature for 2 hours. The reaction was concentrated to dryness under reduced pressure and dried under vacuum to give compound H (66 mg, yield 98.5%) as a white solid, which was used directly in the next step without further purification. LC/MS (ESI) m/z: 322 (M+H)⁺.

Step 7: (2S,4R)-N-((4-carbamimidoylthiophen-2-yl)methyl)-4-(cyclopropylmethoxy)-1-((4-phenoxybenzoyl)glycyl)pyrrolidine-2-carboxamide (Compound 50). To a mixture of compound H (66 mg, 0.2 mmol) and compound I (56 mg, 0.2 mmol) in DMF (2 mL) was added DIPEA (0.2 mL, 1.2 mmol) and T₃P (382 mg, 0.6 mmol, 50% wt in EtOAc) at 0° C. under N2 atmosphere and the mixture was stirred at 35° C. for 5 hours. The mixture was diluted with water and extracted with CHCl₃/i-PrOH (v/v=3/1) three times. The combined organic layers were washed with saturated aq. NaHCO₃, dried over anhydrous Na₂SO₄, filtered, and concentrated to dryness under reduced pressure. The residue was purified by prep-HPLC to give Compound 50 (3 mg, yield 2.50%) as a white solid. ¹H NMR (400 MHz, CD₃OD) δ 8.20 (d, J=1.6 Hz, 1H), 7.84 (d, J=8.8 Hz, 2H), 7.44-7.39 (m, 3H), 7.20 (t, J=7.6 Hz, 1H), 7.07-7.04 (m, 2H), 7.02-6.98 (m, 2H), 4.57 (s, 2H), 4.50 (t, J=8.0 Hz, 1H), 4.28 (s, 1H), 4.21 (s, 2H), 3.85-3.73 (m, 2H), 3.35 (d, J=2.4 Hz, 2H), 2.43-2.34 (m, 1H), 2.11-2.02 (m, 1H), 1.08-0.99 (m, 1H), 0.57-0.50 (m, 2H), 0.25-0.19 (m, 2H); LC/MS (ESI) m/z: 576 (M+H)⁺.

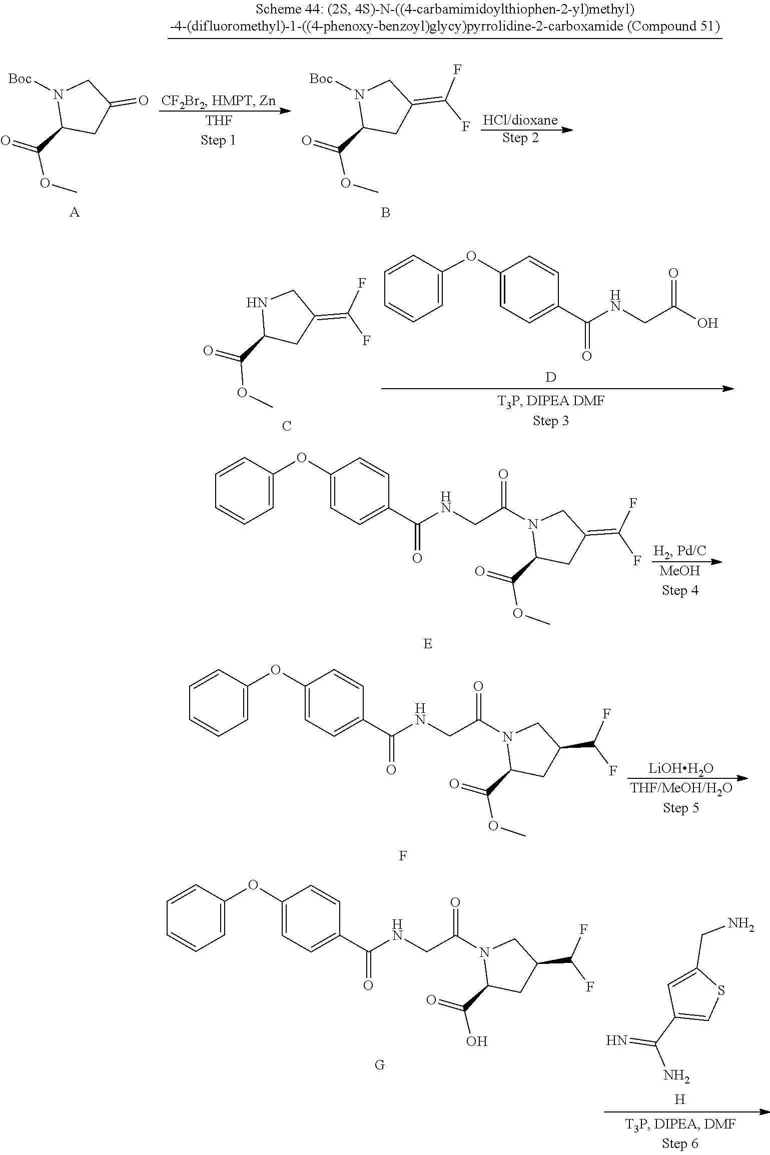

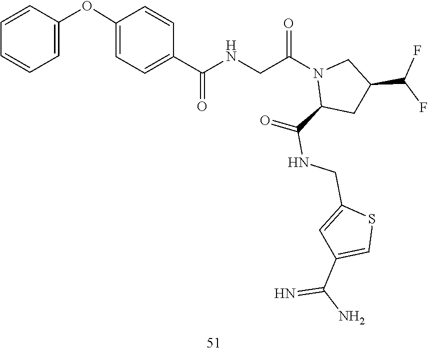 [SMILES: N=C(N)c1csc(CNC(=O)[C@@H]2C[C@H](C(F)F)CN2C(=O)CNC(=O)c2ccc(Oc3ccccc3)cc2)c1]

Step 1: 1-(tert-butyl) 2-methyl (S)-4-(difluoromethylene)pyrrolidine-1,2-dicarboxylate (B).

To a solution of compound A (2 g, 8.23 mmol) in THE (20 mL) was added CF₂Br₂ (3 mL, 32.9 mmol) and HMPT (6 mL, 32.9 mmol) at 0° C. under N₂ atmosphere. The mixture was warmed to room temperature and Zn (2.1 g, 32.9 mmol) and HMPT (0.4 mL) were added. The reaction was stirred at 65° C. for 3.5 hours and the mixture was filtered through Celite and washed with MTBE. The filtrate was washed with water and the aqueous layer was extracted with MTBE twice. The combined organic layers were washed with brine, dried over anhydrous Na₂SO₄, filtered, and concentrated to dryness under reduced pressure. The residue was purified by flash chromatography on silica gel (PE:EtOAc=10:1) to give compound B (1.02 g, yield 44.7%) as an oil. LC/MS (ESI) m/z: 178 (M+H−Boc)⁺.

Step 2: methyl (S)-4-(difluoromethylene)pyrrolidine-2-carboxylate (C). A mixture of compound B (500 mg, 1.8 mmol) in HCl/1,4-dioxane (5 mL) was stirred at room temperature for 2 hours. The reaction mixture was concentrated to dryness under reduced pressure and dried under vacuum to give compound C (319 mg, yield 99.8%) as a yellow oil, which was used directly in the next step without further purification. LC/MS (ESI) m/z: 178 (M+H)⁺.

Step 3: methyl (S)-4-(difluoromethylene)-1-((4-phenoxybenzoyl)glycyl)pyrrolidine-2-carboxylate (E). To a mixture of compound C (319 mg, 1.8 mmol) and D (488 mg, 1.8 mmol) in DMF (3 mL) was added DIPEA (1.8 mL, 10.8 mmol) and T₃P (458 mg, 0.72 mmol, 50% wt in EtOAc) at 0° C. under N2 atmosphere. The resulting mixture was stirred at 30° C. for 16 hours. The mixture was diluted with water and extracted with CHCl₃/i-PrOH (v/v=3/1) three times. The combined organic layers were washed with saturated aq. NaHCO₃, dried over anhydrous Na₂SO₄, filtered, and concentrated to dryness under reduced pressure. The residue was purified by chromatography on silica gel (PE:EtOAc=1:1) to give compound E (413 mg, yield 53.2%) as a yellow oil. LC/MS (ESI) m/z: 431 (M+H)⁺.

Step 4: methyl (2S,4S)-4-(difluoromethyl)-1-((4-phenoxybenzoyl)glycyl)pyrrolidine-2-carboxylate (F). To a solution of compound E (413 mg, 0.96 mmol) in MeOH (4 mL) was added Pd/C (145 mg, 10% wt) at room temperature under N2 atmosphere and the reaction mixture was stirred at room temperature under H₂ atmosphere for 2.5 hours. The mixture was filtered and concentrated to dryness under reduced pressure to give compound F (411 mg, yield 99.0%) as a yellow oil. LC/MS (ESI) m/z: 433 (M+H)⁺.

Step 5: (2S,4S)-4-(difluoromethyl)-1-((4-phenoxybenzoyl)glycyl)pyrrolidine-2-carboxylic acid (G). To a solution of compound F (277 mg, 0.64 mmol) in MeOH (2 mL) and THE (1 mL) was added a solution of LiOH·H₂O (25 mg, 0.64 mmol) in H₂O (1 mL) at 0° C. and the mixture was stirred at 25° C. for 3.5 hours. The mixture was concentrated in vacuo, diluted with H₂O and washed with EtOAc twice. The water layer were acidified with 0.5 M aq. HCl to pH˜3 and extracted with CHCl₃/i-PrOH (v/v=3/1) three times. The combined organic layers were dried over anhydrous Na₂SO₄, filtered, and concentrated to dryness under reduced pressure to give compound G (220 mg, yield 82.1%) as an oil, which was used directly in the next step without further purification. LC/MS (ESI) m/z: 419 (M+H)⁺.

Step 6: (2S,4S)-N-((4-carbamimidoylthiophen-2-yl)methyl)-4-(difluoromethyl)-1-((4-phenoxybenzoyl)glycyl)pyrrolidine-2-carboxamide (Compound 51). To a mixture of compound G (148 mg, 0.24 mmol) and H (55 mg, 0.29 mmol) in DMF (1 mL) was added DIPEA (0.25 mL, 1.44 mmol) and T₃P (458 mg, 0.72 mmol, 50% wt in EtOAc) at 0° C. under N2 atmosphere and the mixture was stirred at 35° C. for 5 hours. The mixture was diluted with water and extracted with CHCl₃/i-PrOH (v/v=3/1) three times. The combined organic layers were washed with saturated aq.NaHCO₃, dried over anhydrous Na₂SO₄, filtered, and concentrated to dryness under reduced pressure. The residue was purified by prep-HPLC to give Compound 51 (37 mg, yield 27.8%) as a white solid. ¹H NMR (400 MHz, CD₃OD) δ 8.53 (s, 1H), 8.23 (d, J=19.6 Hz, 1H), 7.84 (d, J=8.8 Hz, 2H), 7.41 (t, J=8.0 Hz, 3H), 7.20 (t, J=7.4 Hz, 1H), 7.06 (d, J=7.6 Hz, 2H), 7.00 (dd, J=7.6, 8.8 Hz, 2H), 6.15-5.83 (m, 1H), 4.60 (d, J=21.6 Hz, 2H), 4.50 (t, J=8.0 Hz, 1H), 4.32-4.08 (m, 2H), 4.07-3.97 (m, 1H), 3.79-3.59 (m, 1H), 3.05-2.76 (m, 1H), 2.64-2.28 (m, 1H), 2.14-1.88 (m, 1H); LC/MS (ESI) m/z: 556 (M+H)⁺.

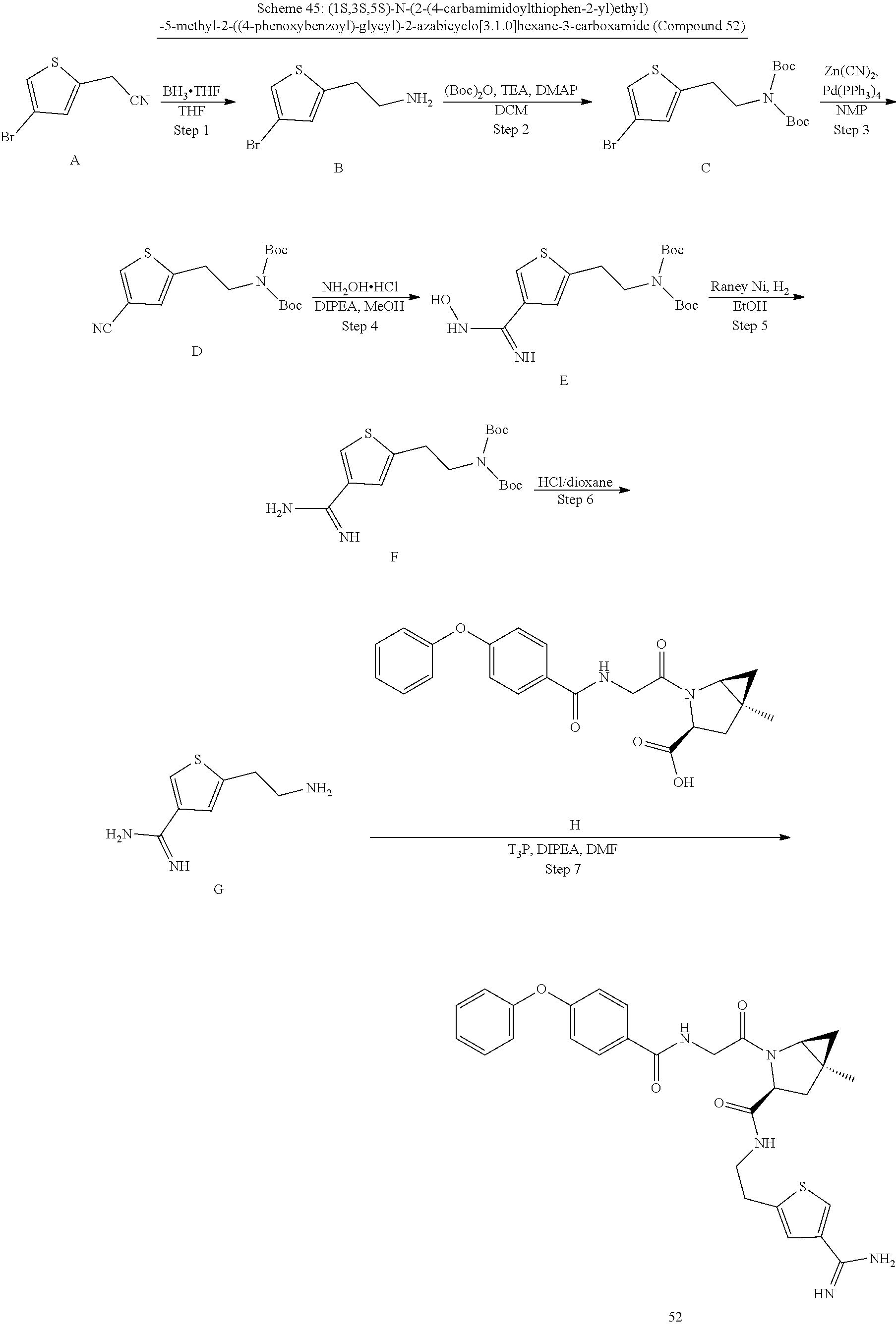

Step 1: 2-(4-bromothiophen-2-yl)ethan-1-amine (B). To a solution of compound A (1 g, 4.97 mmol) in THE (5 mL) was added BH₃THF (2.60 mL, 5.2 mmol) at 0° C. under N2 atmosphere and the mixture was stirred at 65° C. for 2 hours. 0.5 N aq. HCl (20 mL) was added and the mixture was stirred at 65° C. for another 30 minutes. The mixture was cooled down to room temperature and 6 N aq. HCl was added. The mixture was washed with isopropyl ether and the organic layers were discarded. The aqueous layer was neutralized with NaOH and extracted with EtOAc twice. The combined organic layers were washed with brine, dried over anhydrous Na₂SO₄, filtered, and concentrated to dryness under reduced pressure to give compound B (1 g, yield 97.5%) as a yellow oil, which was used directly in the next step without further purification. LC/MS (ESI) m/z: 205 (M+H)⁺.

Step 2: tert-butyl (2-(4-bromothiophen-2-yl)ethyl)(tert-butoxycarbonyl)carbamate (C). To a solution of compound B (1 g, 4.87 mmol) in DCM (15 mL) was added TEA (2.75 mL, 19.6 mmol), DMAP (60 mg, 0.49 mmol) and Boc₂O (3.18 g, 14.6 mmol) at 25° C. under N2 atmosphere and the mixture was stirred at 25° C. for 16 hours. The mixture was diluted with H₂O and extracted with DCM twice. The combined organic layers were dried over anhydrous Na₂SO₄, filtered, and concentrated to dryness under reduced pressure. The residue was purified by flash chromatography on silica gel (PE:EtOAc=50:1) to give compound 3 (800 mg, yield 50.0%) as a yellow oil. LC/MS (ESI) m/z: 353 (M+H)⁺.

Step 3: tert-butyl N-(tert-butoxycarbonyl)-N-[2-(4-cyanothiophen-2-yl)ethyl]carbamate (D). To a solution of compound C (800 mg, 1.97 mmol) in DMF (5 mL) was added Zn(CN)₂ (448 mg, 3.84 mmol) and Pd(PPh₃)₄ (344 mg, 0.296 mmol) at 25° C. under N2 atmosphere and the mixture was stirred at 160° C. for 3 hours. The mixture was cooled down to 25° C., poured into ice-water and extracted with EtOAc twice. The combined organic layers were washed with brine, dried over anhydrous Na₂SO₄, filtered, and concentrated to dryness under reduced pressure. The residue was purified by flash chromatography on silica gel (PE:EtOAc=30:1) to give compound D (700 mg, yield 82%) as a yellow oil. LC/MS (ESI) (m/z): 353 (M+H)⁺.

Step 4: tert-butyl N-(tert-butoxycarbonyl)-N-{2-[4-(N-hydroxycarbamimidoyl)thiophen-2-yl]ethyl}carbamate (E). To a solution of compound D (500 mg, 1.42 mmol) in MeOH (5 mL) was added NH₂OH·HCl (229 mg, 3.55 mmol) and DIPEA (0.70 mL, 4.26 mmol at 25° C. under N2 atmosphere and the mixture was stirred at 25° C. for 16 hours. The reaction was concentrated to dryness under reduced pressure and the residue was purified by flash chromatography on silica gel (PE:EtOAc=20:1) to give compound E (440 mg, yield 80.5%) as a colorless oil. LC/MS (ESI) m/z: 386 (M+H)⁺.

Step 5: tert-butyl N-(tert-butoxycarbonyl)-N-[2-(4-carbamimidoylthiophen-2-yl)ethyl]-carbamate (F). To a solution of compound E (440 mg, 1.14 mmol) in MeOH (5 mL) was added Raney Ni (20 mg) and AcOH (0.4 mL) at 25° C. under N2 atmosphere and the mixture was stirred at 25° C. for 16 hours. The mixture was filtered and the filtrate was concentrated to dryness under reduced pressure to give compound F (170 mg, yield 40.3%) as a light green solid, which was used directly in next step without further purification. LC/MS (ESI) (m/z): 370 (M+H)⁺.

Step 6: 5-(2-aminoethyl)thiophene-3-carboximidamide (G). A mixture of compound F (170 mg, 0.46 mmol) and HCl/1,4-dioxane (2.0 mL) was stirred at room temperature for 1 hour. The reaction mixture was concentrated to dryness under reduced pressure and dried under vacuum to give compound G (70 mg, yield 90.9%) as a colorless oil, which was used directly in the next step without further purification. LC/MS (ESI) m/z: 170 (M+H)⁺.

Step 7: (1S,3S,5S)-N-(2-(4-carbamimidoylthiophen-2-yl)ethyl)-5-methyl-2-((4-phenoxy-benzoyl)glycyl)-2-azabicyclo[3.1.0]hexane-3-carboxamide (Compound 52). To a mixture of compound G (30 mg, 0.18 mmol) and compound H (70 mg, 0.18 mmol) in DMF (5 mL) was added DIPEA (0.17 mL, 1.08 mmol) and T₃P (339 mg, 0.54 mmol, 50% wt. in EtOAc) at 0° C. under N2 atmosphere and the mixture was stirred at 25° C. for 16 hours. The mixture was diluted with H₂O and extracted with CHCl₃/i-PrOH(v:v=3/1) three times. The combined organic layers were dried over anhydrous Na₂SO₄, filtered, and concentrated to dryness under reduced pressure. The residue was purified by flash chromatography on silica gel (DCM:MeOH=5:1) and further purified by prep-HPLC to give Compound 52 (3 mg, yield 3.0%) as a white solid. ¹H NMR (400 MHz, CD₃OD) δ 8.04 (d, J=1.4 Hz, 1H), 7.88-7.81 (m, 2H), 7.45-7.38 (m, 2H), 7.33 (d, J=1.2 Hz, 1H), 7.21 (t, J=7.4 Hz, 1H), 7.07 (dd, J=5.4, 3.4 Hz, 2H), 7.03-6.98 (m, 2H), 4.78 (dd, J=11.4, 3.1 Hz, 1H), 4.31 (q, J=16.3 Hz, 2H), 3.54-3.35 (m, 3H), 3.07 (t, J=6.4 Hz, 2H), 2.36 (t, J=13.0 Hz, 1H), 2.15 (dd, J=13.4, 3.1 Hz, 1H), 1.28 (s, 3H), 1.05 (dd, J=5.8, 2.3 Hz, 1H), 0.78 (t, J=5.4 Hz, 1H); LC/MS (ESI) m/z: 546 (M+H)⁺.

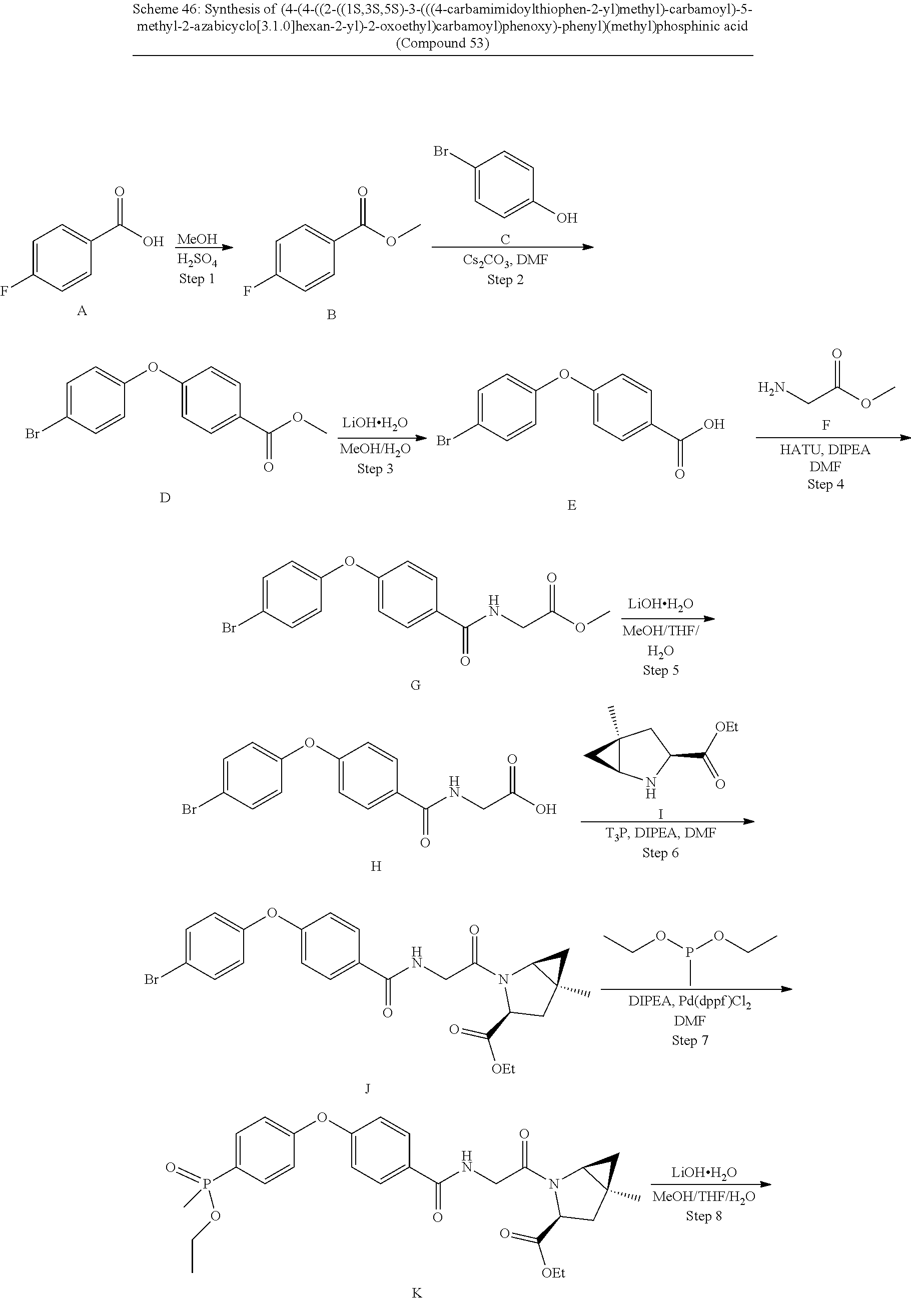

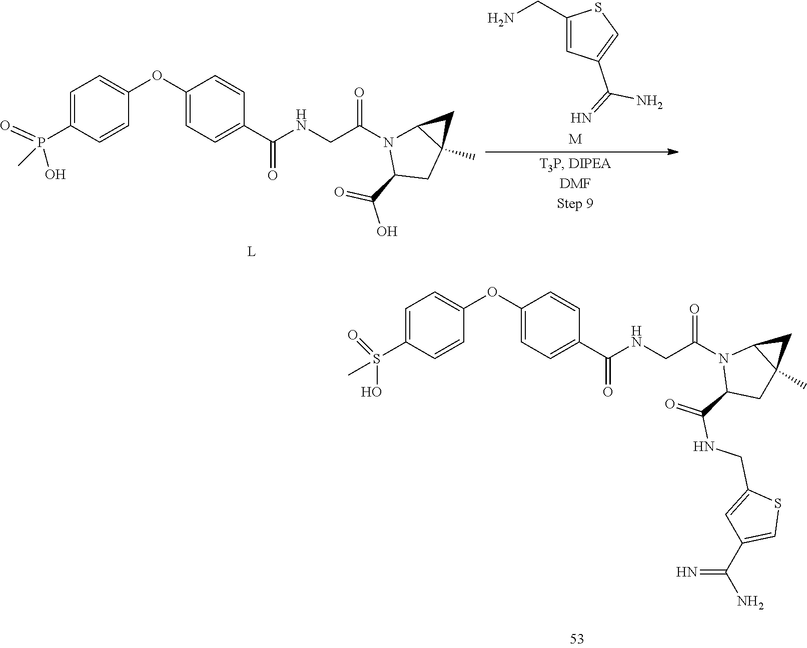

Step 1: methyl 4-fluorobenzoate (B). To a solution of compound A (5 g, 35.7 mmol) in MeOH (70 mL) was added H₂SO₄ (1.0 mL) at 0° C. and the mixture was stirred at 70° C. for 16 hours. The mixture was cooled to 25° C. and concentrated to dryness under reduced pressure. The residue was purified by flash chromatography on silica gel (PE:EtOAc=25:1) to give compound B (5.45 g, yield 99.1%) as a yellow oil.

Step 2: methyl 4-(4-bromophenoxy)benzoate (C). To a mixture of compound B (2 g, 13.0 mmol) and compound C (2.46 g, 14.3 mmol) in DMF (40 mL) was added Cs₂CO₃ (21.2 g, 65.0 mmol) at 25° C. The reaction was stirred at 120° C. for 16 hours and cooled down to 25° C. The mixture was diluted with water and extracted with EtOAc twice. The combined organic layers were washed with saturated aq.NH₄Cl solution and brine, dried over anhydrous Na₂SO₄, filtered, and concentrated to dryness under reduced pressure. The residue was purified by flash chromatography on silica gel (PE:EtOAc=9:1) to give compound D (3.8 g, yield 95.7%) as a brown solid.

Step 3: 4-(4-bromophenoxy)benzoic acid (E). To a solution of compound D (3.8 g, 12.4 mmol) in MeOH (20 mL), THF (10 mL) and water (10 mL) was added LiOH·H₂O (2.6 g, 61.9 mmol) at 0° C. and the mixture was stirred at room temperature for 2 hours. The mixture was concentrated to 1/5 volume, diluted with water and washed with MTBE twice. The aqueous layer was acidified with 1 N aq. HCl to pH˜3 and extracted with DCM twice. The combined organic layers were washed with brine, dried over anhydrous Na₂SO₄, filtered, and concentrated to dryness under reduced pressure to give compound E (3.6 g, yield 99.1%) as a white solid, which was used directly in next step without further purification. LC/MS (ESI) (m/z): 291/293 (M−H)⁻.

Step 4: methyl (4-(4-bromophenoxy)benzoyl)glycinate (G). To a mixture of compound E (2.6 g, 8.90 mmol) and methyl glycinate (F; 1.68 g, 13.4 mmol) in DMF (50 mL) was added DIPEA (7.3 mL, 44.5 mmol) and HATU (6.8 g, 17.8 mmol) at 0° C. under N2 atmosphere and the mixture was stirred at room temperature for 2 hours. The mixture was diluted with water and extracted with EtOAc twice. The combined organic layers were washed with saturated aq.NH₄Cl solution and brine, dried over anhydrous Na₂SO₄, filtered, and concentrated to dryness under reduced pressure. The residue was purified by flash chromatography on silica gel (PE:EtOAc=3:2) to give compound G (3.1 g, yield 96.9%) as a yellow solid. LC/MS (ESI) m/z: 364/366 (M+H)⁺.

Step 5: (4-(4-bromophenoxy)benzoyl)glycine (H). To a solution of compound G (3.1 g, 8.5 mmol) in MeOH (20 mL), THE (10 mL) and water (10 mL) was added LiOH·H₂O (1.8 g, 42.7 mmol) at 0° C. and the mixture was stirred at room temperature for 2 hours. The mixture was concentrated to 1/5 volume, diluted with water, and washed with MTBE twice. The aqueous layer was acidified with 1 N aq. HCl to pH˜3 and extracted with DCM twice. The combined organic layers were washed with brine, dried over anhydrous Na₂SO₄, filtered, and concentrated to dryness under reduced pressure to give compound H (2.9 g, yield 97.3%) as a yellow solid, which was used directly in next step without further purification. LC/MS (ESI) (m/z): 350/352 (M+H)⁺.

Step 6: ethyl (1S,3S,5S)-2-((4-(4-bromophenoxy)benzoyl)glycyl)-5-methyl-2-azabicyclo-[3.1.0]hexane-3-carboxylate (J). To a mixture of compound H (248 mg, 0.71 mmol) and 1 (100 mg, 0.59 mmol) in DMF (5 mL) was added DIPEA (0.58 mL, 3.54 mmol) and T₃P (1.13 g, 1.77 mmol, 50% wt in EtOAc) at 0° C. under N2 atmosphere and the mixture was stirred at room temperature for 16 hours. The mixture was diluted with water and extracted with EtOAc twice. The combined organic layers were washed with saturated aq.NH₄Cl solution and brine, dried over anhydrous Na₂SO₄, filtered and concentrated to dryness under reduced pressure. The residue was purified by flash chromatography on silica gel (PE:EtOAc=1:1) to give compound J (188 mg, yield 63.5%) as a brown semi-solid. LC/MS (ESI) m/z: 501/503 (M+H)⁺.

Step 7: ethyl (1S,3S,5S)-2-((4-(4-(ethoxy(methyl)phosphoryl)phenoxy)benzoyl)glycyl)-5-methyl-2-azabicyclo[3.1.0]hexane-3-carboxylate (K). To a mixture of compound J (60 mg, 0.12 mmol) and DIPEA (0.1 mL, 0.6 mmol) in DMF (1 mL) was added diethyl methylphosphonite (81.6 mg, 0.6 mmol) and Pd(dppf)Cl₂ (4.4 mg, 0.006 mmol) at 25° C. under N2 atmosphere. The mixture was stirred at 130° C. in a microwave reactor for 4 hours. The mixture was cooled down to 25° C. and diluted with H₂O. The mixture was extracted with EtOAc twice. The combined organic layers were washed with brine, dried over anhydrous Na₂SO₄, filtered, and concentrated to dryness under reduced pressure. The residue was purified by flash chromatography on silica gel (PE:EtOAc=100:1 to 3:1) to give compound K (50 mg, yield 79.4%) as a yellow solid. LC/MS (ESI) m/z: 529 (M+H)⁺.

Step 8: (1S,3S,5S)-2-((4-(4-(hydroxy(methyl)phosphoryl)phenoxy)benzoyl)glycyl)-5-methyl-2-azabicyclo[3.1.0]hexane-3-carboxylic acid (L). To a solution of compound K (50 mg, 0.09 mmol) in MeOH (2 mL), THE (1 mL) and water (1 mL) was added LiOH·H₂O (12 mg, 0.27 mmol) at 0° C. and the mixture was stirred at room temperature for 2 hours. The mixture was concentrated to 1/5 volume, diluted with water, and washed with MTBE twice. The aqueous layer was acidified with 1 N aq. HCl to pH˜3 and extracted with CHCl₃/i-PrOH(v/v=3/1) three times. The combined organic layers were dried over anhydrous Na₂SO₄, filtered and concentrated to dryness under reduced pressure to give compound L (40 mg, yield 88.9%) as a yellow solid, which was used directly in the next step without further purification. LC/MS (ESI) (m/z): 473 (M+H)⁺.

Step 9: (4-(4-((2-((1S,3S,5S)-3-(((4-carbamimidoylthiophen-2-yl)methyl)carbamoyl)-5-methyl-2-azabicyclo[3.1.0]hexan-2-yl)-2-oxoethyl)carbamoyl)phenoxy)phenyl)-(methyl)phosphinic acid (Compound 53). To a mixture of compound L (40 mg, 0.08 mmol) and M (20 mg, 0.13 mmol) in DMF (5 mL) was added DIPEA (0.08 mL, 0.48 mmol) and T₃P (81 mg, 0.24 mmol, 50% wt in EtOAc) at 0° C. under N2 atmosphere and the mixture was stirred at room temperature for 16 hours. The mixture was diluted with water and acidified with 1 N aq. HCl to pH˜3, extracted with CHCl₃/i-PrOH (v/v=3/1) three times. The combined organic layers were dried over anhydrous Na₂SO₄, filtered and concentrated to dryness under reduced pressure. The residue was purified by flash chromatography on silica gel (DCM:MeOH=4:1) and further purified by prep-HPLC to give Compound 53 (7 mg, yield 13.5%) as a white solid. ¹H NMR (400 MHz, CD₃OD) δ 8.53 (s, 1H), 8.21 (d, J=1.6 Hz, 1H), 8.05-7.99 (m, 2H), 7.93 (d, J=8.7 Hz, 2H), 7.41 (s, 1H), 7.24-7.19 (m, 2H), 7.18-7.13 (m, 2H), 4.58 (s, 1H), 4.55 (d, J=3.0 Hz, 2H), 4.36 (q, J=16.5 Hz, 2H), 3.42 (dd, J=6.0, 2.4 Hz, 1H), 3.16 (s, 4H), 2.42 (t, J=13.0 Hz, 1H), 2.17 (dd, J=13.4, 3.3 Hz, 1H), 1.30 (s, 3H), 1.15 (dd, J=5.6, 2.5 Hz, 1H), 0.81 (t, J=5.4 Hz, 1H); LC/MS (ESI) m/z: 610 (M+H)⁺.

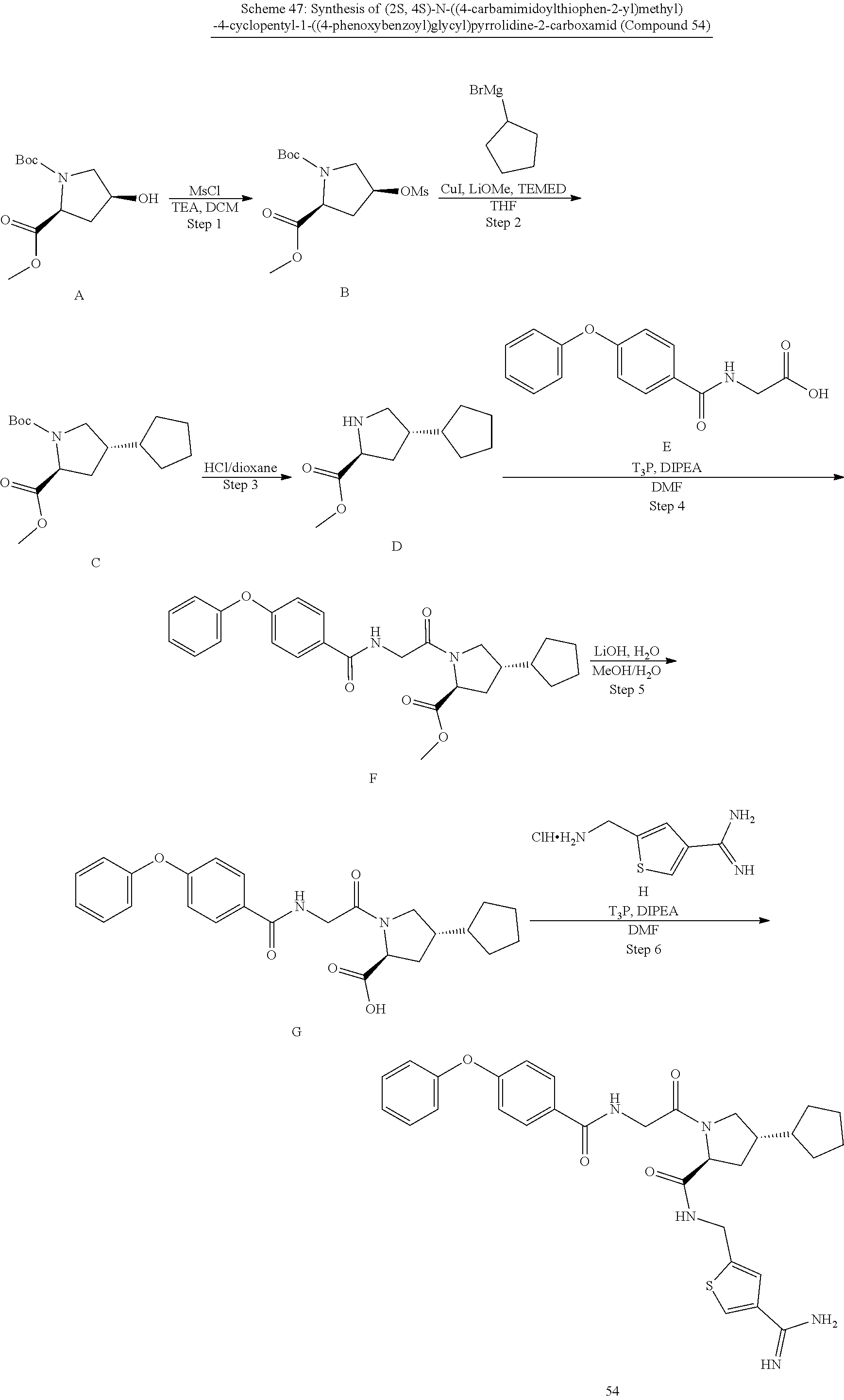

Step 1: 1-(tert-butyl) 2-methyl (2S,4S)-4-((methylsulfonyl)oxy)pyrrolidine-1,2-dicarboxylate (B). To a solution of compound A (2.0 g, 8.15 mmol) in DCM (20 mL) was added TEA (3.29 g, 32.62 mmol) and MsCl (1.87 g, 16.31 mmol) at 0° C. under N₂ atmosphere and the mixture was stirred at 0° C. for 1 hour. The reaction was quenched with ice-water and the resulting mixture was extracted with DCM twice. The combined organic layers were washed with water (2×20 mL) and brine (2×20 mL), dried over anhydrous Na₂SO₄, filtered, and concentrated under reduced pressure to give compound 2 (2.64 g, 100% yield) as a colorless oil, which was used directly in the next step without further purification.

Step 2: 1-(tert-butyl) 2-methyl (2S,4S)-4-cyclopentylpyrrolidine-1,2-dicarboxylate (C). To a mixture of CuI (177 mg, 0.93 mmol) and LiOMe (352 mg, 9.28 mmol) in THE (10 mL) was added compound B (1.5 g, 13.92 mmol) in THE (4 mL), TEMED (52 mg, 0.45 mmol) and bromo(cyclopentyl)magnesium in THE (14 mL, 13.92 mmol, 1 mol/mL) at 0° C. under N₂ atmosphere. The mixture was stirred at 0° C. overnight and the reaction was quenched with saturated aq. NH₄Cl solution. The resulting mixture was extracted with ethyl acetate (3×20 mL). The combined organic layers were washed with brine, dried over anhydrous Na₂SO₄, filtered, and concentrated to dryness under reduced pressure. The residue was purified by column chromatography on silica gel (PE:EtOAc=100:1 to 15:1) to give compound C (80 mg, 5.8% yield) as a yellow oil. LC/MS (ESI) m/z: 198 (M+H−100)⁺.

Step 3: methyl (2S,4S)-4-cyclopentylpyrrolidine-2-carboxylate (D). A mixture of compound C (80 mg, 0.269 mmol) and HCl/1,4-dioxane (2 mL) was stirred at room temperature for 1 hour. The reaction mixture was concentrated to dryness under reduced pressure, diluted with DCM, and dried under vacuum to give compound D (53.06 mg, 100% yield) as a yellow oil, which was used directly in the next step without further purification. LC/MS (ESI) m/z: 198 (M+H)⁺.

Step 4: methyl (2S,4S)-4-cyclopentyl-1-((4-phenoxybenzoyl)glycyl)pyrrolidine-2-carboxylate (F). To a solution of compound D (53 mg, 0.253 mmol) and compound E (75.6 mg, 0.279 mmol) in DMF (3 mL) was added DIPEA (98.1 mg, 0.76 mmol) and T₃P (322.4 mg, 0.507 mmol, 50% in ethyl acetate). The mixture was stirred at room temperature under N2 atmosphere overnight. The reaction was quenched with saturated aq. NH₄Cl solution. The resulting mixture was extracted with ethyl acetate (3×20 mL). The combined organic layers were washed with water (2×20 mL) and brine (2×20 mL), dried over anhydrous Na₂SO₄, filtered, and concentrated under reduced pressure. The residue was purified by column chromatography on silica gel (PE/EtOAc=5:1 to 1:1) to give compound F (60 mg, 52.5% yield) as a yellow oil. LC/MS (ESI) m/z: 451 (M+H)⁺.

Step 5: (2S,4S)-4-cyclopentyl-1-((4-phenoxybenzoyl)glycyl)pyrrolidine-2-carboxylic acid (G). To a solution of compound F (60 mg, 0.133 mmol) in methanol (1.5 mL) and water (0.5 mL) was added a solution of LiOH·H₂O (5.6 mg, 0.133 mmol) at 0° C. and the mixture was stirred at room temperature for 2 hours. The mixture was acidified with 1 N aq. HCl solution to pH<3 and extracted with dichloromethane (2×10 mL). The combined organic layers were concentrated to dryness under reduced pressure to give compound G (40 mg, 68.8% yield) as a white solid, which was used directly in the next step without further purification. LC/MS (ESI) (m/z): 437 (M+H)⁺.

Step 6: (2S,4S)-N-((4-carbamimidoylthiophen-2-yl)methyl)-4-cyclopentyl-1-((4-phenoxy-benzoyl)glycyl)pyrrolidine-2-carboxamide (Compound 54). To a solution of compound G (40 mg, 0.133 mmol) and compound H (75.6 mg, 0.199 mmol) in DMF (3 mL) was added DIPEA (35.5 mg, 0.76 mmol) and T₃P (116.5 mg, 0.183 mmol, 50% in ethyl acetate). The mixture was stirred at room temperature under N2 atmosphere overnight. The reaction was quenched with saturated aq.NH₄Cl solution. The resulting mixture was extracted with CHCl₃/i-PrOH (v:v=3/1) (3×20 mL). The combined organic layers were washed with water (2×20 mL) and brine (2×20 mL), dried over anhydrous Na₂SO₄, filtered and concentrated under reduced pressure. The residue was purified by prep-HPLC to give Compound 54 (2.0 mg, 3.8% yield). LC/MS(ESI) m/z: 574 (M+H)⁺. ¹H NMR (400 MHz, CD₃OD) δ 8.22 (t, J=8.6 Hz, 1H), 7.84 (d, J=8.7 Hz, 2H), 7.52-7.36 (m, 3H), 7.21 (t, J=7.4 Hz, 1H), 7.07 (d, J=7.7 Hz, 2H), 7.00 (d, J=8.8 Hz, 2H), 4.57 (t, J=9.0 Hz, 3H), 4.26 (dd, J=16.7, 6.8 Hz, 1H), 4.14 (dd, J=16.7, 9.1 Hz, 1H), 3.94 (t, J=8.4 Hz, 1H), 2.15 (dd, J=22.1, 11.0 Hz, 2H), 1.92-1.55 (m, 8H), 1.23 (dd, J=22.3, 15.2 Hz, 3H); LC/MS (ESI) m/z: 574 (M+H)⁺.

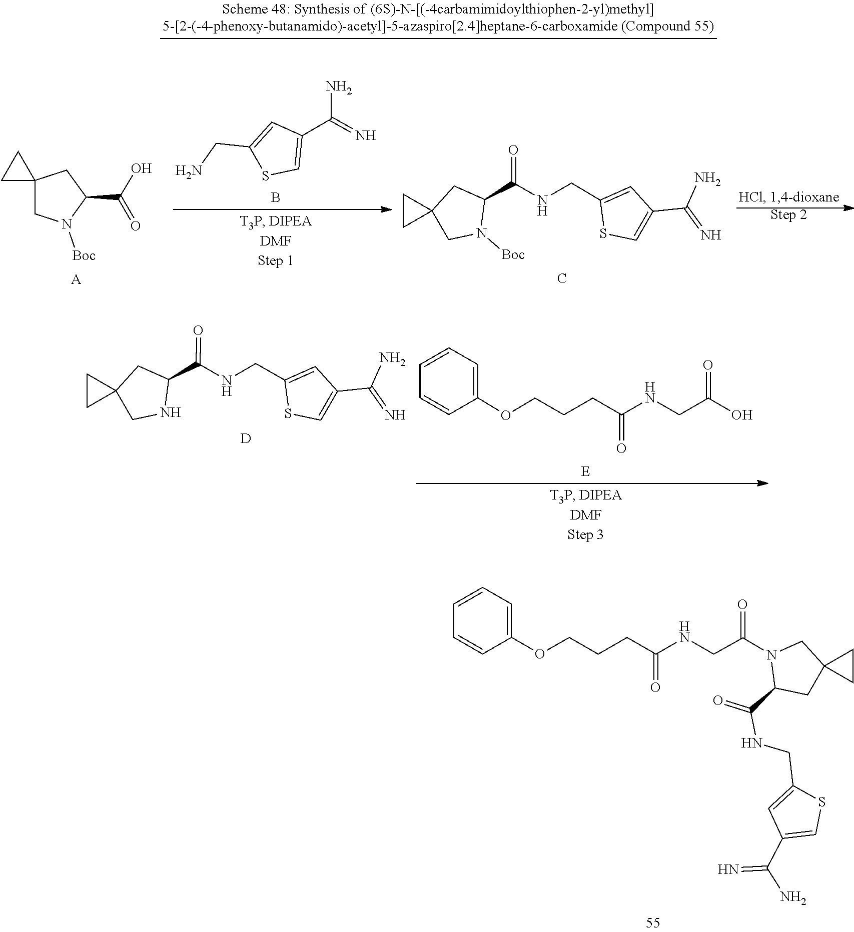

Step 1: tert-butyl(S)-6-(((4-carbamimidoylthiophen-2-yl)methyl)carbamoyl)-5-azaspiro[2.4]heptane-5-carboxylate (C). To a mixture of compound A (300 mg, 1.24 mmol) and compound B (473.6 mg, 2.48 mmol) in DMF (3 mL) was added DIPEA (479 mg, 3.72 mmol) and T₃P (1.2 g, 1.86 mmol, 50% wt in ethyl acetate) at room temperature under N2 atmosphere. The mixture was stirred at room temperature overnight and quenched with saturated aqueous NH₄Cl. The resulting mixture was extracted with ethyl acetate twice. The combined organic layers were washed with water and brine, dried over anhydrous Na₂SO₄, filtered, and concentrated to dryness. The residue was purified by prep-HPLC to give compound C (231 mg, 49.3% yield). LC/MS (ESI) m/z: 379 (M+H)⁺.

Step 2: (S)-N-((4-carbamimidoylthiophen-2-yl)methyl)-5-azaspiro[2.4]heptane-6-carboxamide (D). A mixture of compound C (231 mg, 0.61 mmol) and 4 N HCl/1,4-dioxane (2 mL) was stirred at room temperature for 1 hour. The reaction mixture was concentrated to dryness under reduced pressure, diluted with dichloromethane, and dried under vacuum to give compound D (130 mg, yield 76.9%), which was used directly in the next step without further purification. LC/MS (ESI) m/z: 279 (M+H)⁺.

Step 3: (S)-N-((4-carbamimidoylthiophen-2-yl)methyl)-5-((4-phenoxybutanoyl)glycyl)-5-azaspiro[2.4]heptane-6-carboxamide (Compound 55). To a mixture of compound D (130 mg, 0.41 mmol) and compound E (116.7 mg, 0.49 mmol) in DMF (1 mL) was added DIPEA (158.9 mg, 1.23 mmol) and T₃P (393.7 mg, 0.615 mmol, 50% wt in ethyl acetate) at room temperature under N2 atmosphere. The mixture was stirred at room temperature overnight and quenched with saturated aqueous NH₄Cl. The resulting mixture was extracted with ethyl acetate twice. The combined organic layers were washed with H₂O and brine, dried over anhydrous Na₂SO₄, filtered, and concentrated to dryness. The residue was purified by prep-HPLC to give Compound 55 (2.1 mg, 1.04% yield). ¹H NMR (400 MHz, CD₃OD) δ 8.22 (d, J=1.6 Hz, 1H), 7.42 (s, 1H), 7.26-7.22 (m, 2H), 6.92-6.89 (m, 2H), 6.88 (s, 1H), 4.62-4.55 (m, 3H), 4.03-3.90 (m, 4H), 3.67 (d, J=9.7 Hz, 1H), 3.44 (d, J=9.7 Hz, 1H), 2.47 (t, J=7.4 Hz, 2H), 2.35 (dd, J=12.8, 8.8 Hz, 1H), 2.09-2.04 (m, 2H), 1.78 (dd, J=12.8, 3.3 Hz, 1H), 0.70-0.60 (m, 3H), 0.50 (dd, J=10.0, 7.0 Hz, 1H); LC/MS (ESI) m/z: 498 (M+H)⁺.

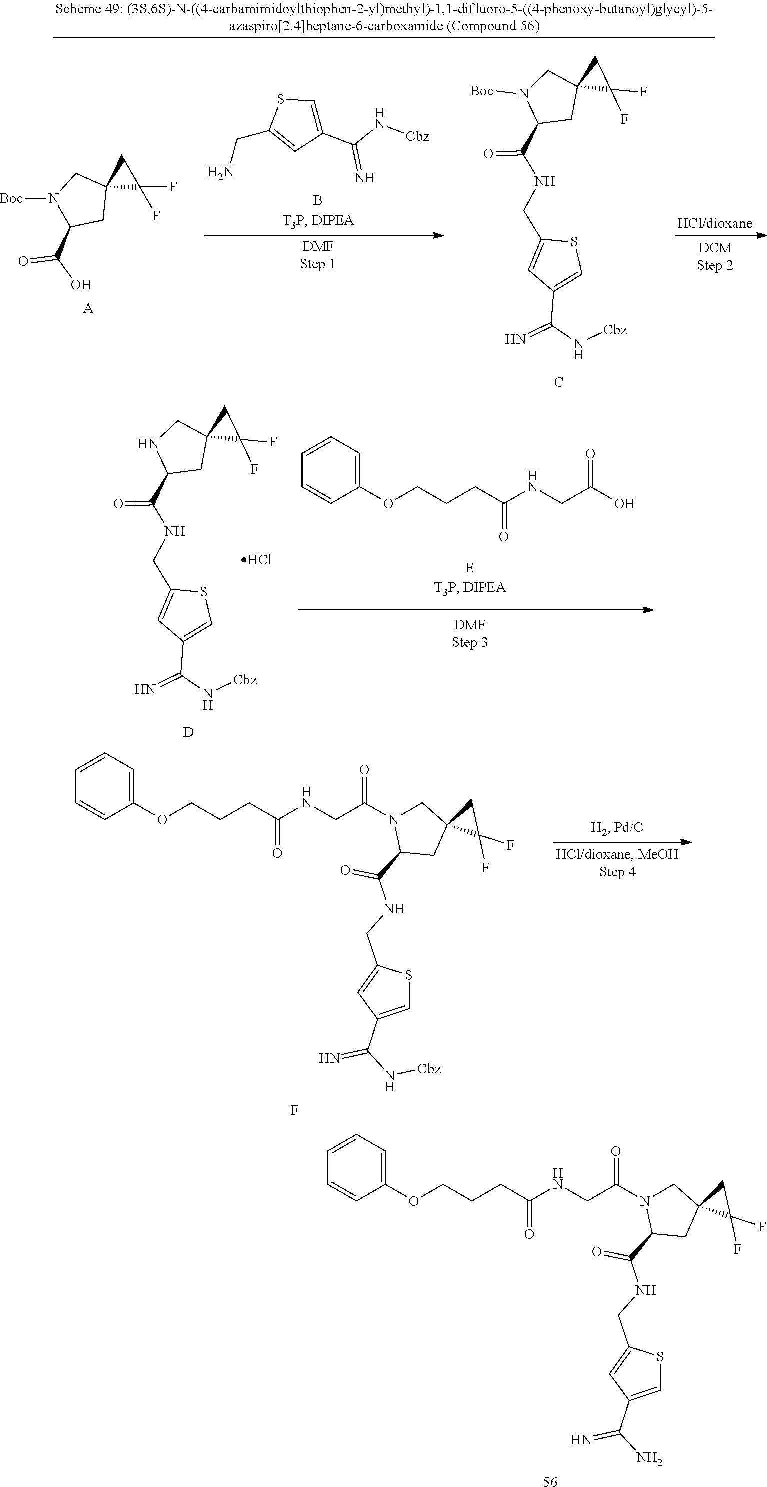

Step 1: tert-butyl (3S,6S)-6-(((4-(N-((benzyloxy)carbonyl)carbamimidoyl)thiophen-2-yl)methyl)carbamoyl)-1,1-difluoro-5-azaspiro[2.4]heptane-5-carboxylate (C). To a mixture of compound A (50 mg, 0.18 mmol) and compound B (64 mg, 0.22 mmol) in DMF (1 mL) was added DIPEA (70 mg, 0.54 mmol) and T₃P (172 mg, 0.27 mmol, 50% wt. in ethyl acetate) at room temperature under N2 atmosphere. The mixture was stirred at room temperature overnight and quenched with saturated aqueous NH₄Cl. The resulting mixture was extracted with ethyl acetate twice. The combined organic layers were washed with H₂O and brine, dried over anhydrous Na₂SO₄, filtered, and concentrated to dryness under reduced pressure. The residue was purified by prep-HPLC to give compound C (49 mg, 49.9% yield). LC/MS (ESI) m/z: 549 (M+H)⁺.

Step 2: benzyl ((5-(((3S,6S)-1,1-difluoro-5-azaspiro[2.4]heptane-6-carboxamido)methyl) thiophen-3-yl)(imino)methyl)carbamate (D). A mixture of compound C (49 mg, 0.089 mmol) and HCl/1,4-dioxane (1 mL) was stirred at room temperature for 1 hour. The reaction mixture was concentrated to dryness under reduced pressure, diluted with dichloromethane, and dried under vacuum to give compound D (40 mg, yield 99.8%), which was used directly in the next step without further purification. LC/MS (ESI) m/z: 449 (M+H)⁺.

Step 3: benzyl ((5-(((3S,6S)-1,1-difluoro-5-((4-phenoxybutanoyl)glycyl)-5-azaspiro[2.4]heptane-6-carboxamido)methyl)thiophen-3-yl)(imino)methyl)carbamate(F). To a mixture of compound D (40 mg, 0.089 mmol) and compound E (25 mg, 0.107 mmol) in DMF (1 mL) was added DIPEA (35 mg, 0.267 mmol) and T₃P (86 mg, 0.134 mmol, 50% wt. in ethyl acetate) at room temperature under N₂ atmosphere. The mixture was stirred at room temperature overnight and quenched with saturated aqueous NH₄Cl. The resulting mixture was extracted with ethyl acetate twice. The combined organic layers were washed with H₂O and brine, dried over anhydrous Na₂SO₄, filtered, and concentrated to dryness under reduced pressure. The residue was purified by prep-HPLC to give compound F (30 mg, 50.37% yield). LC/MS (ESI) m/z: 668 (M+H)⁺.

Step 4: (3S,6S)-N-((4-carbamimidoylthiophen-2-yl)methyl)-1,1-difluoro-5-((4-phenoxy-butanoyl)glycyl)-5-azaspiro[2.4]heptane-6-carboxamide(Compound 56). To a solution of compound F (30 mg, 0.045 mmol) in methanol (1 mL) was added HCl/1,4-dioxane (1 drop) and Pd/C (1 mg) and stirred at room temperature for 3 hours. The reaction was quenched with saturated aqueous NaHCO₃ and the resulting mixture was extracted with ethyl acetate twice. The combined organic layers were washed with H₂O and brine, dried over anhydrous Na₂SO₄, filtered, and concentrated to dryness under reduced pressure. The residue was purified by prep-HPLC to give Compound 56 (7.3 mg, 30.45% yield). ¹H NMR (400 MHz, CD₃OD) δ 8.24 (dd, J=5.1, 1.6 Hz, 1H), 7.45 (t, J=13.2 Hz, 1H), 7.24 (dd, J=9.5, 6.6 Hz, 2H), 6.92-6.89 (m, 2H), 6.88 (d, J=0.9 Hz, 1H), 4.64-4.56 (m, 3H), 4.00 (dd, J=11.5, 6.3 Hz, 4H), 3.91 (d, J=9.9 Hz, 1H), 3.69 (dd, J=10.5, 4.2 Hz, 1H), 2.57 (dd, J=13.0, 9.3 Hz, 1H), 2.47 (t, J=7.4 Hz, 2H), 2.09-2.05 (m, 2H), 2.04-1.97 (m, 1H), 1.56-1.40 (m, 2H); LC/MS (ESI) m/z: 534 (M+H)⁺.

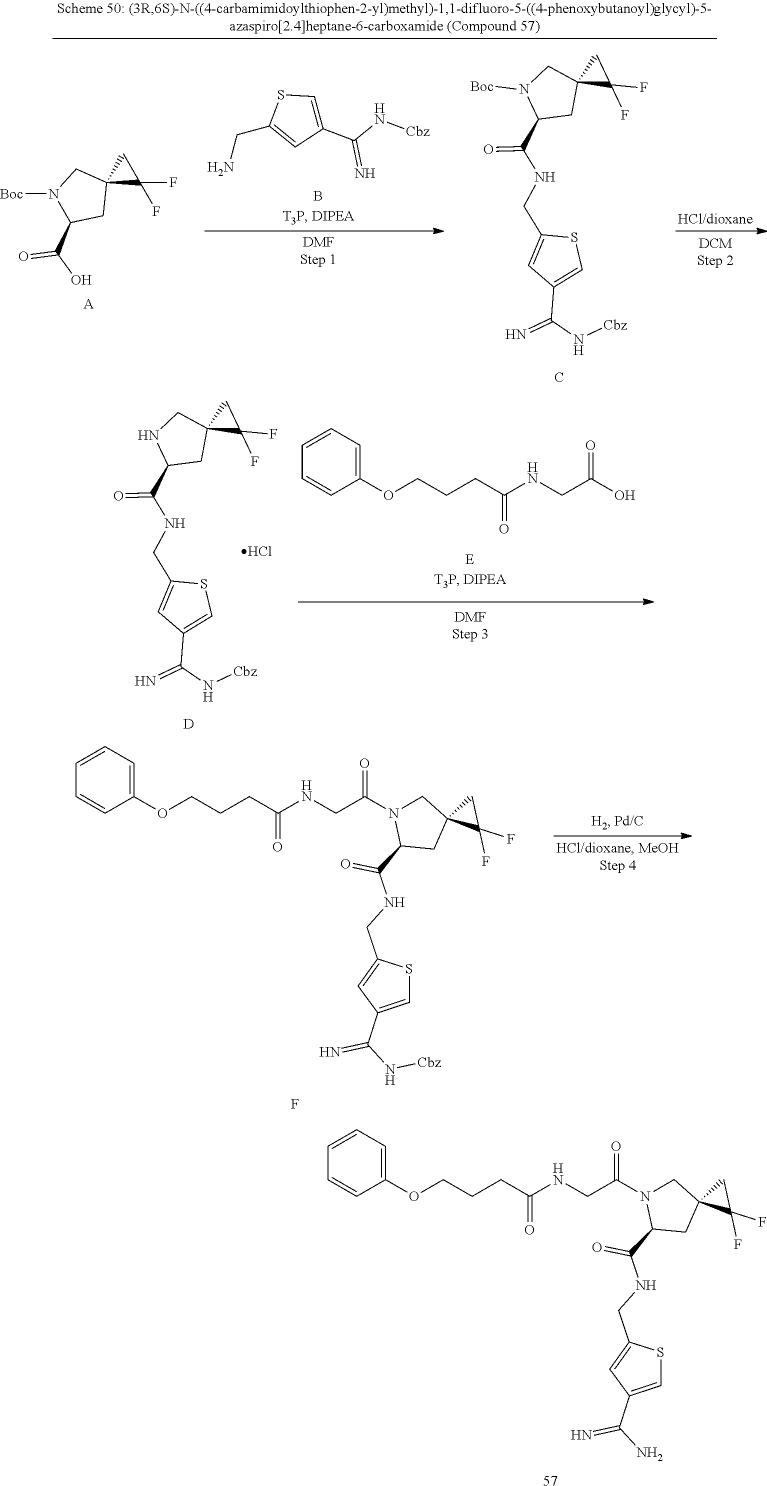

Step 1: tert-butyl (3R,6S)-6-(((4-(N-((benzyloxy)carbonyl)carbamimidoyl)thiophen-2-yl) methyl)carbamoyl)-1,1-difluoro-5-azaspiro[2.4]heptane-5-carboxylate (C). To a mixture of compound A (50 mg, 0.18 mmol) and compound B (64 mg, 0.22 mmol) in DMF (1 mL) was added DIPEA (70 mg, 0.54 mmol) and T₃P (172 mg, 0.27 mmol, 50% wt. in ethyl acetate) at room temperature under N₂ atmosphere. The mixture was stirred at room temperature overnight and quenched with saturated aqueous NH₄Cl. The resulting mixture was extracted with ethyl acetate twice. The combined organic layers were washed with H₂O and brine, dried over anhydrous Na₂SO₄, filtered, and concentrated to dryness. The residue was purified by prep-HPLC to give compound C (30 mg, 30.94% yield). LC/MS (ESI) m/z: 549 (M+H)⁺.

Step 2: benzyl ((5-(((3R,6S)-1,1-difluoro-5-azaspiro[2.4]heptane-6-carboxamido)methyl) thiophen-3-yl)(imino)methyl)carbamate (D). A mixture of compound C (30 mg, 0.055 mmol) and HCl/1,4-dioxane (1 mL) was stirred at room temperature for 1 hour. The reaction mixture was concentrated to dryness under reduced pressure, diluted with DCM, and dried under vacuum to give compound D (23 mg, yield 98.78%), which was used directly in the next step without further purification. LC/MS (ESI) m/z: 449 (M+H)⁺.

Step 3: benzyl ((5-(((3R,6S)-1,1-difluoro-5-((4-phenoxybutanoyl)glycyl)-5-azaspiro[2.4]heptane-6-carboxamido)methyl)thiophen-3-yl)(imino)methyl)carbamate (F). To a mixture of compound D (23 mg, 0.051 mmol) and compound E (14 mg, 0.061 mmol) in DMF (1 mL) was added DIPEA (20 mg, 0.153 mmol) and T₃P (48 mg, 0.077 mmol, 50% w.t in ethyl acetate) at room temperature under N₂ atmosphere. The mixture was stirred at room temperature overnight and quenched with saturated aqueous NH₄Cl. The resulting mixture was extracted with ethyl acetate twice. The combined organic layers were washed with H₂O and brine, dried over anhydrous Na₂SO₄, filtered, and concentrated to dryness under reduced pressure. The residue was purified by prep-HPLC to give compound F (30 mg, 87.60% yield). LC/MS (ESI) m/z: 668 (M+H)⁺.

Step 4: (3R,6S)-N-((4-carbamimidoylthiophen-2-yl)methyl)-1,1-difluoro-5-((4-phenoxy-butanoyl)glycyl)-5-azaspiro[2.4]heptane-6-carboxamide(Compound 58). To a solution of compound F (30 mg, 0.045 mmol) in methanol (1 mL) was added HCl/1,4-dioxane (1 drop) and Pd/C (1 mg) and stirred at room temperature for 3 hours. The reaction was quenched with saturated aqueous NaHCO₃ and the resulting mixture was extracted with ethyl acetate twice. The combined organic layers were washed with H₂O and brine, dried over anhydrous Na₂SO₄, filtered, and concentrated to dryness under reduced pressure. The residue was purified by prep-HPLC to give Compound 57 (3.4 mg, 21.3% yield). ¹H NMR (400 MHz, CD₃OD) δ 8.24 (dd, J=5.1, 1.6 Hz, 1H), 7.45 (t, J=13.2 Hz, 1H), 7.24 (dd, J=9.5, 6.6 Hz, 2H), 6.92-6.89 (m, 2H), 6.88 (d, J=0.9 Hz, 1H), 4.64-4.56 (m, 3H), 4.00 (dd, J=11.5, 6.3 Hz, 4H), 3.91 (d, J=9.9 Hz, 1H), 3.69 (dd, J=10.5, 4.2 Hz, 1H), 2.57 (dd, J=13.0, 9.3 Hz, 1H), 2.47 (t, J=7.4 Hz, 2H), 2.09-2.05 (m, 2H), 2.04-1.97 (m, 1H), 1.56-1.40 (m, 2H); LC/MS (ESI) m/z: 534 (M+H)⁺.

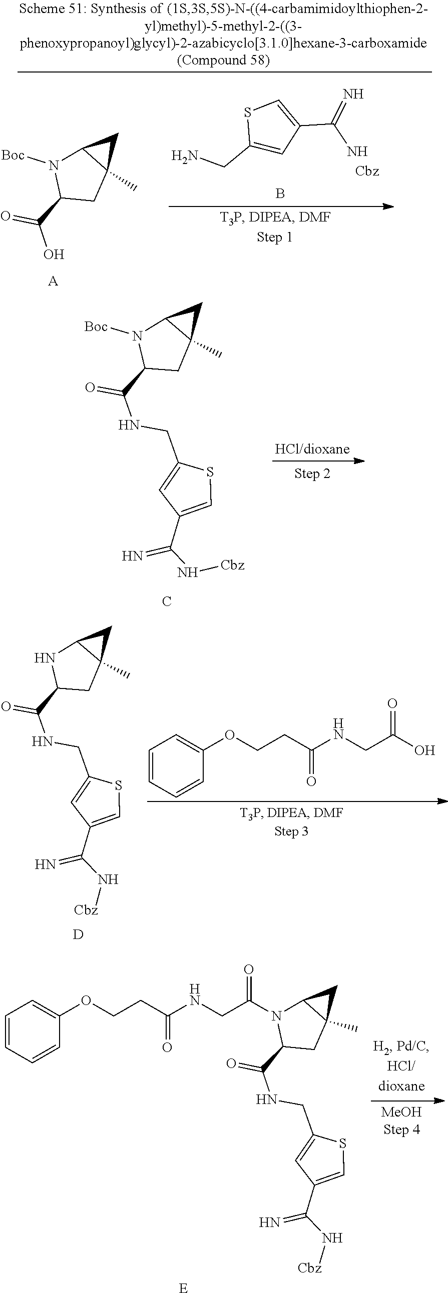

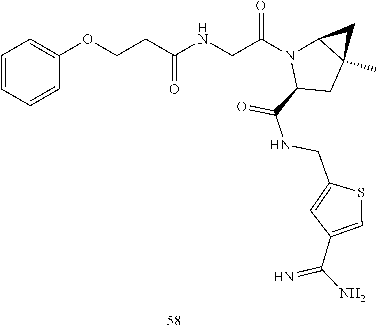 [SMILES: C[C@@]12C[C@@H]1N(C(=O)CNC(=O)CCOc1ccccc1)[C@H](C(=O)NCc1cc(C(=N)N)cs1)C2 ; "58"]

Step 1: tert-butyl (1S,3S,5S)-3-(((4-(N-((benzyloxy)carbonyl)carbamimidoyl)thiophen-2-yl)methyl)carbamoyl)-5-methyl-2-azabicyclo[3.1.0]hexane-2-carboxylate (C). To a mixture of compound A (300 mg, 1.24 mmol) and compound B (360 mg, 1.24 mmol) in DMF (6 mL) was added DIPEA (964 mg, 7.46 mmol) and T₃P (1.187 g, 3.73 mmol, 50% wt. in EtOAc) at 0° C. under N₂ atmosphere and the resulting mixture was stirred at room temperature for 3 hours. The mixture was diluted with water and extracted with EtOAc twice. The combined organic layers combined were washed with brine, dried over anhydrous Na₂SO₄, filtered, and concentrated to dryness under reduced pressure. The residue was purified by flash chromatography on silica gel (DCM:MeOH=100:1 to 20:1) to give compound C (560 mg, yield 88.0%) as a light-yellow oil. LC/MS(ESI) m/z: 513 (M+H)⁺.

Step 2: benzyl (imino(5-(((1S,3S,5S)-5-methyl-2-azabicyclo[3.1.0]hexane-3-carboxamido)-methyl)thiophen-3-yl)methyl)carbamate (D). A mixture of compound 3 (140 mg, 0.27 mmol) in HCl/1,4-dioxane (2 mL, 4 M) was stirred at room temperature for 1 hour. The mixture was concentrated to dryness under reduced pressure, co-evaporated with DCM twice and dried under vacuum to give compound D (110 mg, yield 97.6%) as a yellow oil, which was used directly in the next step without further purification. LC/MS(ESI) m/z: 413 (M+H)⁺.

Step 3: benzyl (imino(5-(((1S,3S,5S)-5-methyl-2-((3-phenoxypropanoyl)glycyl)-2-azabicyclo-[3.1.0]hexane-3-carboxamido)methyl)thiophen-3-yl)methyl)carbamate (E). To a mixture of compound D (40 mg, 0.097 mmol) and (3-phenoxypropanoyl)glycine (22 mg, 0.097 mmol) in DMF (2 mL) was added DIPEA (75 mg, 0.10 mmol) and T₃P (93 g, 0.29 mmol, 50% wt in EtOAc) at 0° C. under N₂ atmosphere and the resulting mixture was stirred at room temperature overnight. The mixture was diluted with water and extracted with EtOAc twice. The combined organic layers were washed with brine, dried over anhydrous Na₂SO₄, filtered and concentrated to dryness under reduced pressure. The residue was purified by flash chromatography on silica gel (DCM:MeOH=100:1 to 15:1) to give compound 5 (30 mg, yield 50.1%) as a light-yellow oil. LC/MS(ESI) m/z: 618 (M+H)⁺.

Step 4: (1S,3S,5S)-N-((4-carbamimidoylthiophen-2-yl)methyl)-5-methyl-2-((3-phenoxy-propanoyl)glycyl)-2-azabicyclo[3.1.0]hexane-3-carboxamide (Compound 58). To a solution of compound E (30 mg, 0.049 mmol) in MeOH (3 mL) was added Pd/C (10 mg, 10% wt) and HCl/1,4-dioxane (1 drop) at room temperature under N₂ atmosphere and the mixture was stirred at room temperature under H₂ atmosphere for 3 hours. The mixture was filtered and the filtrate was concentrated to dryness under reduced pressure. The residue was purified by prep-HPLC to Compound 58 (3 mg, yield 12.8%) as a white solid. ¹H NMR (400 MHz, CD₃OD) δ 8.21 (d, J=1.6 Hz, 1H), 7.41 (s, 1H), 7.29-7.21 (m, 2H), 6.92 (t, J=8.4 Hz, 3H), 4.82-4.78 (m, 1H), 4.56-4.43 (m, 2H), 4.28-4.14 (m, 4H), 3.36-3.33 (m, 1H), 2.74 (t, J=6.2 Hz, 2H), 2.39 (t, J=11.8 Hz, 1H), 2.15 (dd, J=13.2, 13.2 Hz, 1H), 1.28 (s, 3H), 1.14 (dd, J=5.6, 5.6 Hz, 1H), 0.81 (t, J=5.4 Hz, 1H); LC/MS(ESI) m/z: 484 (M+H)⁺.

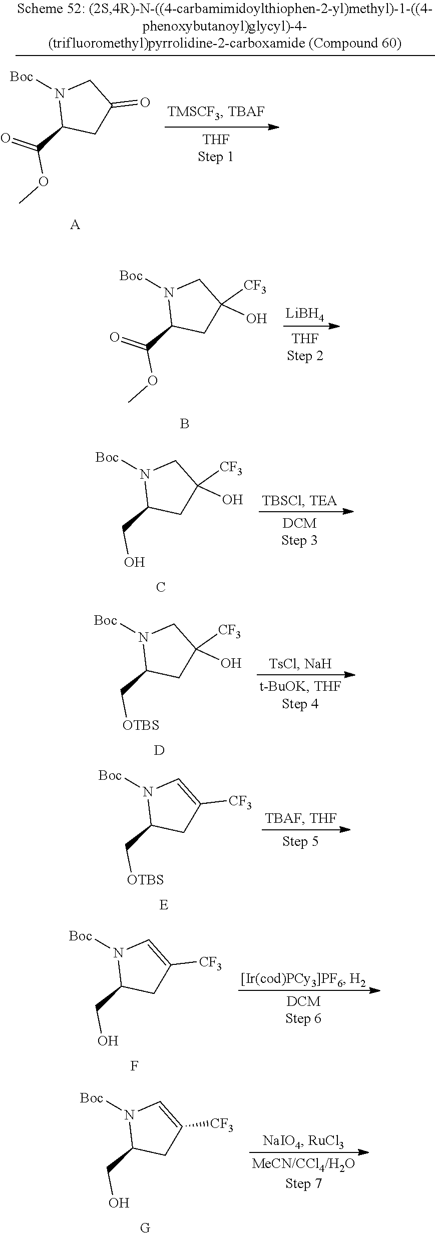

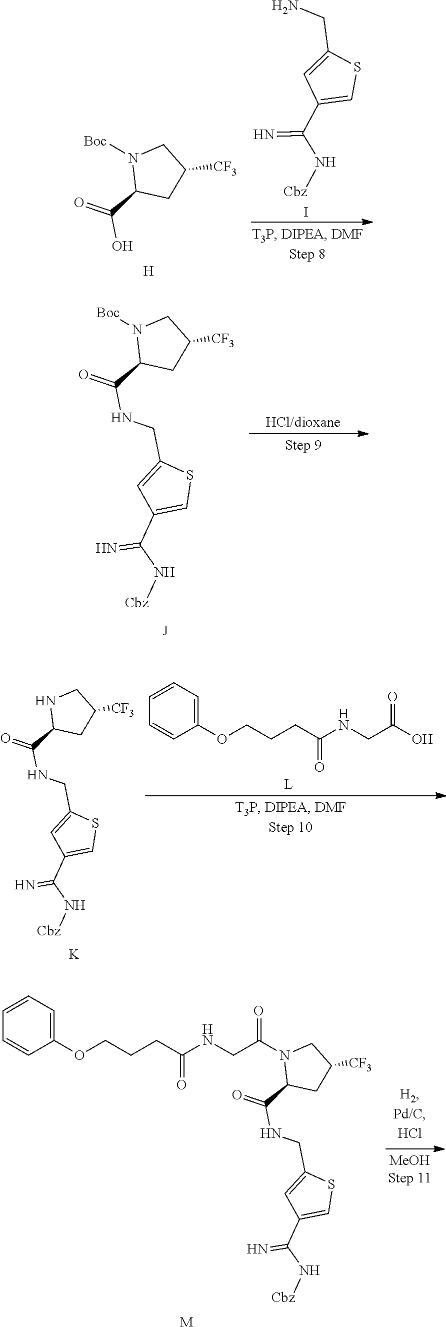

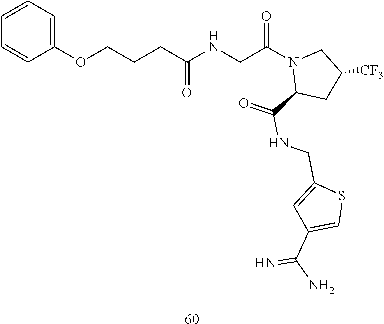 [SMILES: N=C(N)c1csc(CNC(=O)[C@@H]2C[C@@H](C(F)(F)F)CN2C(=O)CNC(=O)CCCOc2ccccc2)c1 ; "60"]

Step 1: (2S)-1-tert-butyl 2-methyl 4-hydroxy-4-(trifluoromethyl)pyrrolidine-1,2-dicarboxylate (B). To a solution of compound A (3 g, 12.4 mmol) in THF (30 mL) was added TMSCF₃ (3.6 mL, 24.8 mmol) and TBAF (323 mg, 1.24 mmol) at 0° C. under N₂ atmosphere. The reaction was stirred at 25° C. for 16 hours and additional TBAF (6.5 g, 24.8 mmol) was added. The mixture was stirred at 25° C. for another 3 hours and concentrated to dryness under reduced pressure. The residue was diluted with water and extracted with DCM twice. The combined organic layers were washed with brine, dried over anhydrous Na₂SO₄, filtered, and concentrated to dryness under reduced pressure. The residue was purified by flash chromatography on silica gel (PE:EtOAc=6:1 to 1:1) to give compound B (2.25 g, yield 57.7%) as a colorless oil. LC/MS (ESI) m/z: 214 (M+H−Boc)⁺.

Step 2: (2S)-tert-butyl 4-hydroxy-2-(hydroxymethyl)-4-(trifluoromethyl)pyrrolidine-1-carboxylate (C). To a solution of compound B (2.25 g, 7.2 mmol) in THF (30 mL) was added LiBH₄ (7.2 mL, 14.4 mmol, 2.0 M in THF) at 0° C. under N₂ atmosphere and the reaction was stirred at 25° C. for 2 hours. The reaction was quenched with saturated aq. NH₄Cl and extracted with EtOAc twice. The combined organic layers were washed with brine, dried over anhydrous Na₂SO₄, filtered, and concentrated to dryness under reduced pressure. The residue was purified by flash chromatography on silica gel (PE:EtOAc=10:1 to 5:1) to give compound C (1.9 g, yield 92.6%) as a colorless oil. LC/MS (ESI) m/z: 186 (M+H−Boc)⁺.

Step 3: (2S)-tert-butyl 2-(((tert-butyldimethylsilyl)oxy)methyl)-4-hydroxy-4-(trifluoromethyl)-pyrrolidine-1-carboxylate (D). To a solution of compound C (1.9 g, 6.70 mmol) in DCM (19 mL) was added TEA (1.86 mL, 13.4 mmol), TBSCl (1.1 g, 7.37 mmol) and DMAP (163 mg, 1.34 mmol) at 0° C. under N₂ atmosphere and the reaction was stirred at 25° C. for 16 hours. The mixture was diluted with water and extracted with DCM twice. The combined organic layers were washed with brine, dried over anhydrous Na₂SO₄, filtered, and concentrated to dryness under reduced pressure. The residue was purified by flash chromatography on silica gel (PE:EtOAc=10:1) to give compound D (2.3 g, yield 86.0%) as a white solid. LC/MS (ESI) m/z: 300 (M+H−Boc)⁺.

Step 4: (S)-tert-butyl 2-(((tert-butyldimethylsilyl)oxy)methyl)-4-(trifluoromethyl)-2,3-dihydro-1H-pyrrole-1-carboxylate (E). To a solution of compound D (2 g, 5.00 mmol) was added NaH (340 mg, 8.50 mmol, 60% dispersion in mineral oil) at 0° C. under N₂ atmosphere and the mixture was stirred at 0° C. for 20 minutes. TsCl (1.43 g, 7.50 mmol) was added to the mixture at 0° C. and the reaction was stirred at 25° C. for 1 hour. The mixture was cooled down to −78° C. and t-BuOK (10 ml, 10.0 mmol, 1.0 M in THF) was added. The reaction was stirred at 25° C. for another 2 hours and quenched with water at 0° C. The mixture was extracted with EtOAc twice and the combined organic layers were washed with brine, dried over anhydrous Na₂SO₄, filtered, and concentrated to dryness under reduced pressure. The residue was purified by flash chromatography on silica gel (PE:EtOAc=100:1 to 20:1) to give compound E (1.7 g, yield 89.2%) as a yellow oil. LC/MS (ESI) m/z: 282 (M+H−Boc)⁺.

Step 5: (S)-tert-butyl 2-(hydroxymethyl)-4-(trifluoromethyl)-2,3-dihydro-1H-pyrrole-1-carboxylate (F). To a solution of compound E (700 mg, 1.84 mmol) in THF (7 mL) was added TBAF (1.2 g, 3.68 mmol) at 0° C. and the reaction was stirred at room temperature for 3 hours. The mixture was diluted with water and extracted with EtOAc twice. The combined organic layers were washed with brine, dried over anhydrous Na₂SO₄, filtered, and concentrated to dryness under reduced pressure. The residue was purified by flash chromatography on silica gel (PE:EtOAc=3:1) to give compound F (386 mg, yield 78.6%) as a light-yellow oil. LC/MS (ESI) m/z: 168 (M+H−Boc)⁺.

Step 6: (2S,4R)-tert-butyl 2-(hydroxymethyl)-4-(trifluoromethyl)pyrrolidine-1-carboxylate (G). To a solution of compound F (156 mg, 0.58 mmol) in DCM (3 mL) was added [Ir(COD)Py3]PF₆ (9 mg, 0.01 mmol) at 25° C. under N₂ atmosphere. The reaction was stirred at 25° C. under H₂ atmosphere for 4 hours. The mixture was filtered and concentrated to dryness under reduced pressure. The residue was purified by flash chromatography on silica gel (PE:EtOAc=5:1) to give compound G (123 mg, yield 78.3%) as an oil. LC/MS (ESI) m/z: 214 (M+H−56)⁺.

Step 7: (2S,4R)-1-(tert-butoxycarbonyl)-4-(trifluoromethyl)pyrrolidine-2-carboxylic acid (H). To a solution of compound H (113 mg, 0.42 mmol) in MeCN (1 mL), CCl₄ (1 mL) and H₂O (1.5 mL) was added NaIO₄ (360 mg, 1.68 mmol) at 0° C. The resulting mixture was stirred at 25° C. for 0.5 hour and RuCl₃ (2 mg, 0.008 mmol) was added in one portion. The reaction mixture was stirred for 2 hours while maintaining the temperature from 18° C. to 24° C. The reaction was diluted with water and filtered off. The filtrate was extracted with DCM twice, and the combined organic layers were washed with brine, dried over anhydrous Na₂SO₄, filtered, and concentrated to dryness under reduced pressure. The residue was purified by flash chromatography on silica gel (DCM:MeOH=10:1) to give compound H (45 mg, yield 37.9%) as a yellow oil. LC/MS (ESI) m/z: 228 (M+H−56)⁺.

Step 8: tert-butyl (2S,4R)-2-(((4-(N-((benzyloxy)carbonyl)carbamimidoyl)thiophen-2-yl)methyl)carbamoyl)-4-(trifluoromethyl)pyrrolidine-1-carboxylate (J). To a mixture of compound H (45 mg, 0.16 mmol) and compound I (69 mg, 0.24 mmol) in DMF (2 mL) was added DIPEA (0.2 mL, 0.96 mmol) and T₃P (305 mg, 0.48 mmol, 50% wt in EtOAc) at 25° C. under N₂ atmosphere and the reaction mixture was stirred at 35° C. for 4 hours. The mixture was washed with saturated aq. NaHCO₃ and the aqueous layer was extracted with EtOAc twice. The combined organic layers were dried over anhydrous Na₂SO₄, filtered and concentrated to dryness under reduced pressure. The residue was purified by flash chromatography on silica gel (PE:EtOAc=5:1) to give compound J (82 mg, yield 93.0%) as a yellow oil. LC/MS (ESI) m/z: 555 (M+H)⁺.

Step 9: benzyl(imino(5-(((2S,4R)-4-(trifluoromethyl)pyrrolidine-2-carboxamido)methyl)thio phen-3-yl)methyl)carbamate (K). A mixture of compound J (82 mg, 0.15 mmol) in HCl/1,4-dioxane (2 mL) was stirred at room temperature for 2 hours. The reaction mixture was concentrated to dryness under reduced pressure, co-evaporated with DCM and dried under vacuum to give compound 11 (67 mg, yield 99.7%) as a yellow solid, which was used directly in the next step without further purification. LC/MS (ESI) m/z: 455 (M+H)⁺.

Step 10: benzyl(imino(5-(((2S,4R)-1-((4-phenoxybutanoyl)glycyl)-4-(trifluoromethyl)pyrrolidi ne-2-carboxamido)methyl)thiophen-3-yl)methyl)carbamate (L). To a mixture of compound K (67 mg, 0.15 mmol) and compound L (35 mg, 0.15 mmol) in DMF (3 mL) was added DIPEA (0.15 mL, 0.9 mmol) and T₃P (286 mg, 0.45 mmol, 50% wt in EtOAc) at 0° C. under N₂ atmosphere and the mixture was stirred at 35° C. for 5 hours. The mixture was washed with saturated aq.NaHCO₃ and extracted with EtOAc twice. The combined organic layers were washed with brine, dried over anhydrous Na₂SO₄, filtered, and concentrated to dryness under reduced pressure. The residue was purified by flash chromatography on silica gel (DCM:MeOH=10:1) to give compound L (64 mg, yield 64.4%) as a yellow oil. LC/MS (ESI) m/z: 674 (M+H)⁺.

Step 11: (2S,4R)-N-((4-carbamimidoylthiophen-2-yl)methyl)-1-((4-phenoxybutanoyl) glycyl)-4-(trifluoromethyl)pyrrolidine-2-carboxamide (Compound 60). To a solution of compound M (64 mg, 0.1 mmol) in MeOH (2 mL) was added Pd/C (22 mg, 35% wt) and HCl/1,4-dioxane (0.05 mL, 4.0 M) at 0° C. under N₂ atmosphere and The reaction was stirred at 25° C. under H₂ atmosphere for 16 hours. The mixture was filtered and concentrated to dryness under reduced pressure. The residue was purified by prep-HPLC to give Compound 60 (15.7 mg, yield 30.6%) as a white solid. ¹H NMR (400 MHz, CD₃OD) δ 8.23 (dd, J=5.2, 5.2 Hz, 1H), 7.43 (t, J=14.9 Hz, 1H), 7.27-7.20 (m, 2H), 6.92-6.86 (m, 3H), 4.84-4.72 (m, 1H), 4.69-4.60 (m, 1H), 4.56 (s, 1H), 4.14-3.89 (m, 5H), 3.86-3.69 (m, 2H), 2.50-2.44 (m, 2H), 2.42-2.32 (m, 1H), 2.30-2.20 (m, 1H), 2.11-2.03 (m, 2H); LC/MS (ESI) m/z: 540 (M+H)⁺.

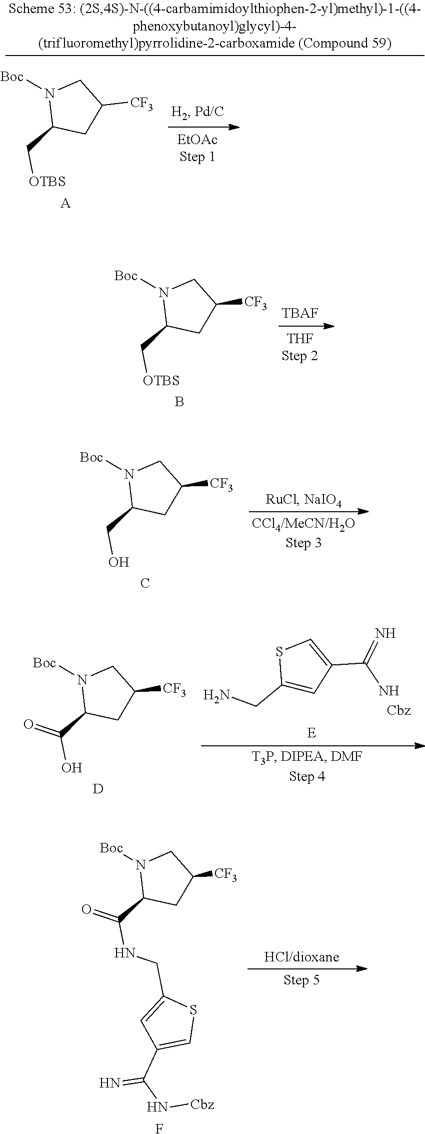

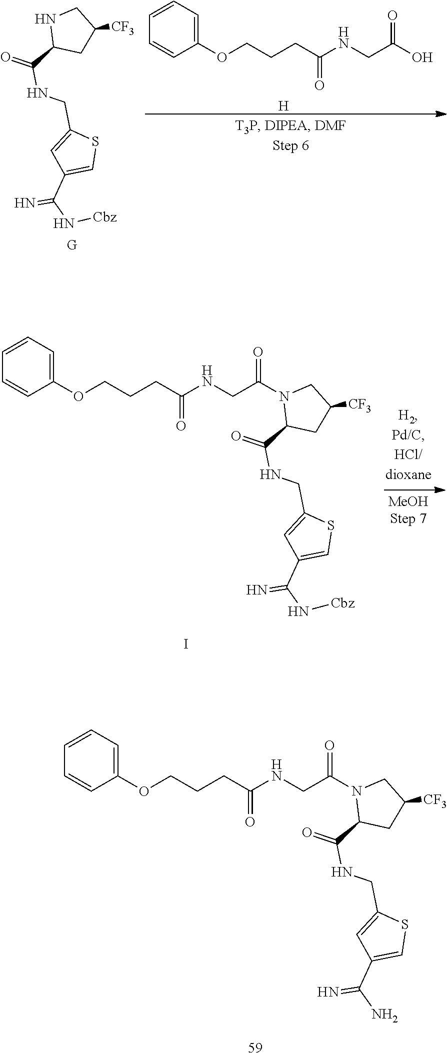

Step 1: tert-butyl (S)-2-(((tert-butyldimethylsilyl)oxy)methyl)-4-(trifluoromethyl)-2,3-dihydro-1H-pyrrole-1-carboxylate (B). To a solution of compound A (500 mg, 1.31 mmol) in EtOAc (5 mL) was added Pd/C (20 mg, 10% wt) at 25° C. under N₂ atmosphere and the mixture was stirred at 25° C. under H₂ atmosphere for 16 hours. The mixture was filtered and the filtrate was concentrated to dryness under reduced pressure. The residue was purified by flash chromatography on silica gel (PE:EtOAc=3:1) to give compound 2 (400 mg, yield 80.3%) as a light-yellow oil. LC/MS (ESI) (m/z): 384 (M+H)⁺.

Step 2: tert-butyl (2S,4S)-2-(hydroxymethyl)-4-(trifluoromethyl)pyrrolidine-1-carboxylate (C). To a solution of compound B (380 mg, 1.0 mmol) in THE (4 mL) was added TBAF (1 mL, 2.0 mmol, 2 M in THF) at 0° C. and the mixture was stirred at 0° C. for 2 hours. The mixture was diluted with H₂O and extracted with EtOAc twice. The combined organic layers were washed with brine, dried over anhydrous Na₂SO₄, filtered, and concentrated to dryness under reduced pressure. The residue was purified by flash chromatography on silica gel (PE:EtOAc=1:1) to give compound C (260 mg, yield 96.3%) as a yellow oil. LC/MS (ESI) m/z: 270 (M+H)⁺.

Step 3: (2S,4S)-1-(tert-butoxycarbonyl)-4-(trifluoromethyl)pyrrolidine-2-carboxylic acid (D). To a solution of compound C (260 mg, 0.96 mmol) in H₂O (1.5 mL), CCl₄ (1 mL) and MeCN (1 mL) was added NaIO₄ (0.823 g, 3.862 mmol) at room temperature. The mixture was stirred at room temperature for 0.5 hour and RuCl₃·3H₂O (0.005 g, 0.019 mmol) was added in one portion. The reaction mixture was stirred for another 2 hours while maintaining the temperature from 18° C. to 24° C. The resulting precipitate is filtered off and washed with DCM. The filtrate was separated and the aqueous layer was extracted with DCM twice. The combined organic layers were dried over anhydrous Na₂SO₄, filtered, and concentrated to dryness under reduced pressure. The residue was purified by flash chromatography on silica gel (DCM:MeOH=100:1 to 10:1) to give compound D (179 mg, yield 65.4%) as yellow oil. LC/MS (ESI) m/z: 284 (M+H)⁺.

Step 4: tert-butyl (2S,4S)-2-(((4-(N-((benzyloxy)carbonyl)carbamimidoyl)thiophen-2-yl)methyl)carbamoyl)-4-(trifluoromethyl)pyrrolidine-1-carboxylate (F). To a mixture of compound D (50 mg, 0.17 mmol) and compound E (75 mg, 0.26 mmol) in DMF (2.0 mL) was added DIPEA (0.2 mL, 1.08 mmol) and T₃P (171 mg, 0.54 mmol, 50% wt. in EtOAc) at 0° C. under N₂ atmosphere and the mixture was stirred at 25° C. for 16 hours. The mixture was diluted with H₂O and extracted with EtOAc twice. The combined organic layers were washed with brine, dried over anhydrous Na₂SO₄, filtered, and concentrated to dryness under reduced pressure. The residue was purified by flash chromatography on silica gel (DCM:MeOH=5:1) and to give compound F (120 mg, yield 70.0%) as a yellow oil. LC/MS (ESI) m/z: 555 (M+H)⁺.

Step 5: benzyl (imino(5-(((2S,4S)-4-(trifluoromethyl)pyrrolidine-2-carboxamido)-methyl)-thiophen-3-yl)methyl)carbamate (G). A mixture of compound F (120 mg, 0.21 mmol) and HCl/1,4-dioxane (1 mL) was stirred at room temperature for 1 hour. The reaction mixture was concentrated to dryness under reduced pressure, co-evaporated with DCM twice, and dried under vacuum to give compound G (100 mg, yield 96.0%) as a colorless oil, which was used directly in the next step without further purification. LC/MS (ESI) m/z: 455 (M+H)⁺.

Step 6: benzyl (imino(5-(((2S,4S)-1-((4-phenoxybutanoyl)glycyl)-4-(trifluoro-methyl)-pyrrolidine-2-carboxamido)methyl)thiophen-3-yl)methyl)carbamate (1). To a mixture of compound G (70 mg, 0.15 mmol) and compound H (36 mg, 0.15 mmol) in DMF (2.0 mL) was added DIPEA (0.30 mL, 0.45 mmol) and T₃P (286 mg, 0.45 mmol, 50% wt. in EtOAc) at 0° C. under N₂ atmosphere and the mixture was stirred at 25° C. for 16 hours. The mixture was diluted with H₂O and extracted with EtOAc twice. The combined organic layers were dried over anhydrous Na₂SO₄, filtered, and concentrated to dryness under reduced pressure. The residue was purified by flash chromatography on silica gel (DCM:MeOH=10:1) to give compound I (100 mg, yield 99.2%) as yellow solid. LC/MS (ESI) m/z: 674 (M+H)⁺.

Step 7: (2S,4S)-N-((4-carbamimidoylthiophen-2-yl)methyl)-1-((4-phenoxybutanoyl)glycyl)-4-(trifluoromethyl)pyrrolidine-2-carboxamide (Compound 59). To a solution of compound I (100 mg, 0.15 mmol) in MeOH (2.0 mL) was added Pd/C (20 mg, 10% wt) and HCl/1,4-dioxane (1 drop) at 25° C. under N₂ atmosphere and the mixture was stirred at 25° C. under H₂ atmosphere for 16 hours. The mixture was filtered and the filtrate was concentrated to dryness under reduced pressure. The residue was purified by prep-HPLC to give Compound 61 (5.0 mg, yield 4.0%) as a white solid. ¹H NMR (400 MHz, CD₃OD) δ 8.23 (dd, J=6.0, 1.6 Hz, 1H), 7.42 (s, 1H), 7.26-7.20 (m, 2H), 6.92-6.86 (m, 3H), 4.87-4.86 (m, 1H), 4.58 (d, J=21.2 Hz, 2H), 4.50 (t, J=8.4 Hz, 1H), 4.09 (dd, J=17.7, 5.7 Hz, 2H), 4.03-3.94 (m, 3H), 3.66 (t, J=10.1 Hz, 1H), 2.58 (dt, J=13.1, 8.1 Hz, 1H), 2.46 (t, J=7.4 Hz, 2H), 2.13-1.97 (m, 3H); LC/MS (ESI) m/z: 540 (M+H)⁺.

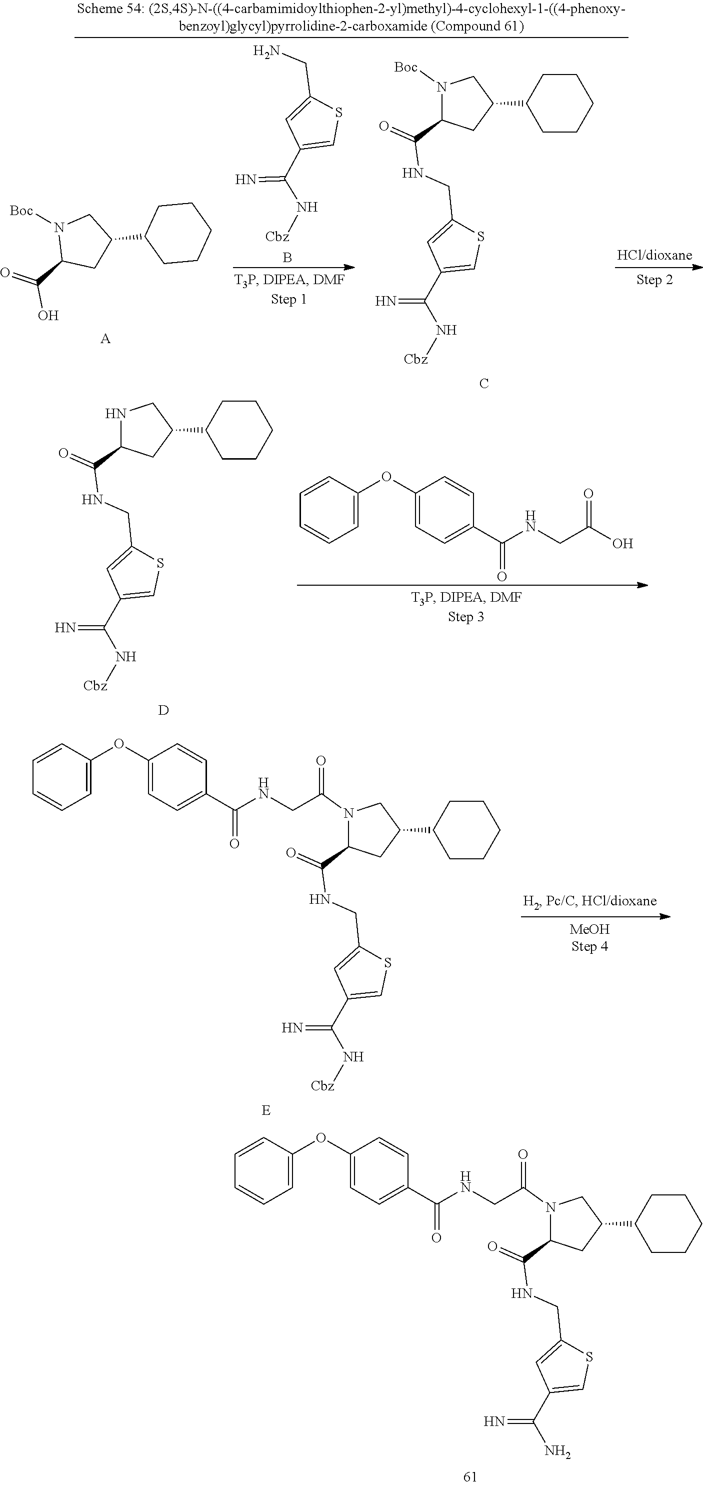

Step 1: tert-butyl (2S,4S)-2-(((4-(N-((benzyloxy)carbonyl)carbamimidoyl)thiophen-2-yl)methyl)carbamoyl)-4-cyclohexylpyrrolidine-1-carboxylate (C). To a mixture of compound A (50 mg, 0.17 mmol) and compound B (49 mg, 0.17 mmol) in DMF (1 mL) was added DIPEA (0.2 mL, 1.02 mmol) and T₃P (324 mg, 0.51 mmol, 50% wt in EtOAc) at 0° C. under N₂ atmosphere and the mixture was stirred at 35° C. for 5 hours. The mixture was diluted with EtOAc and washed with saturated aq. NaHCO₃ solution. The aqueous layer was extracted with EtOAc twice and the combined organic layers were washed with brine, dried over anhydrous Na₂SO₄, filtered and concentrated to dryness under reduced pressure. The residue was purified by flash chromatography on silica gel (PE:EtOAc=1:1) to give compound C (30 mg, yield 31.4%) as a yellow oil. LC/MS (ESI) m/z: 569 (M+H)⁺.

Step 2: benzyl ((5-(((2S,4S)-4-cyclohexylpyrrolidine-2-carboxamido)methyl)thiophen-3-yl)(imino)methyl)carbamate (D). A mixture of compound C (30 mg, 0.05 mmol) in HCl/1,4-dioxane (2 mL) was stirred at room temperature for 2 hours. The mixture was concentrated to dryness under reduced pressure, co-evaporated with DCM twice, and dried under vacuum to give compound D (21 mg, yield 87.5%) as a yellow oil, which was used directly in the next step without further purification. LC/MS (ESI) m/z: 469 (M+H)⁺.

Step 3: benzyl ((5-(((2S,4S)-4-cyclohexyl-1-((4-phenoxybenzoyl)glycyl)pyrrolidine-2-carboxamido)methyl)thiophen-3-yl)(imino)methyl)carbamate (E). To a mixture of compound D (21 mg, 0.04 mmol) and (4-phenoxybenzoyl)glycine (12 mg, 0.04 mmol) in DMF (1 mL) was added DIPEA (0.04 mL, 0.24 mmol) and T₃P (76 mg, 0.12 mmol, 50% wt in EtOAc) at 0° C. under N₂ atmosphere and the mixture was stirred at 35° C. for 3.5 hours. The mixture was diluted with EtOAc and washed with saturated aq.NaHCO₃ solution. The aqueous layer was extracted with EtOAc twice and the combined organic layers were washed with brine, dried over anhydrous Na₂SO₄, filtered, and concentrated to dryness under reduced pressure. The residue was purified by flash chromatography on silica gel (DCM:MeOH=10:1) to give compound E (20 mg, yield 63.1%) as a yellow oil. LC/MS (ESI) m/z: 722 (M+H)⁺.

Step 4: (2S,4S)-N-((4-carbamimidoylthiophen-2-yl)methyl)-4-cyclohexyl-1-((4-phenoxy-benzoyl)glycyl)pyrrolidine-2-carboxamide (Compound 61). To a solution of compound E (20 mg, 0.03 mmol) in MeOH (2 mL) was added Pd/C (7 mg, 10% wt.) and HCl/1,4-dioxane (2 drops) at room temperature under N₂ atmosphere and the reaction mixture was stirred at room temperature under a H₂ balloon for 16 hours. The mixture was filtered and the filtrate was concentrated to dryness under reduced pressure. The residue was purified by prep-HPLC to give Compound 61 (5.1 mg, yield 31.3%) as a white solid. ¹H NMR (400 MHz, CD₃OD) δ 8.53 (s, 1H), 8.23 (s, 1H), 7.85-7.80 (m, 2H), 7.44-7.37 (m, 3H), 7.21 (t, J=7.4 Hz, 1H), 7.08-7.04 (m, 2H), 7.02-6.97 (m, 2H), 4.87-4.85 (m, 1H), 4.57 (d, J=9.6 Hz, 2H), 4.53 (d, J=6.0 Hz, 1H), 4.27 (d, J=16.8 Hz, 2H), 4.11 (d, J=16.8 Hz, 2H), 3.99-3.91 (m, 1H), 2.29-2.07 (m, 2H), 1.89-1.66 (m, 6H), 1.33-1.20 (m, 4H), 1.09-0.94 (m, 2H). LC/MS (ESI) m/z: 588 (M+H)⁺.

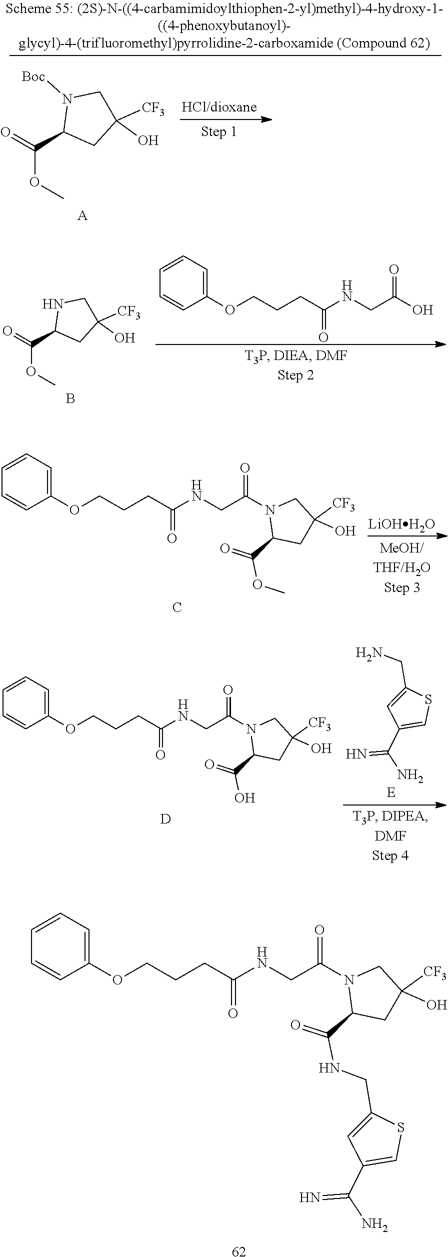

Step 1: methyl (2S)-4-hydroxy-4-(trifluoromethyl)pyrrolidine-2-carboxylate hydrochloride (B). A mixture of compound A (90 mg, 0.29 mmol) in HCl/1,4-dioxane (2 mL) was stirred at room temperature for 3 hours. The reaction mixture was concentrated to dryness under reduced pressure, co-evaporated with DCM twice and dried under vacuum to give compound B (59 mg, yield 96.7%) as a yellow oil, which was used directly in the next step. LC/MS (ESI) m/z: 214 (M+H)⁺.

Step 2: methyl (2S)-4-hydroxy-1-((4-phenoxybutanoyl)glycyl)-4-(trifluoromethyl)pyrrolidine-2-carboxylate (C). To a mixture of compound B (55 mg, 0.24 mmol) and (4-phenoxybutanoyl)glycine (56 mg, 0.24 mmol) in DMF (3 mL) was added DIPEA (0.24 mL, 1.44 mmol) and T₃P (458 mg, 0.72 mmol, 50% wt. in EtOAc) at 0° C. under N₂ atmosphere and the mixture was stirred at 35° C. for 5 hours. The mixture was diluted with EtOAc and washed with saturated aq. NaHCO₃ solution. The aqueous layer was extracted with EtOAc twice and the combined organic layers were washed with brine, dried over anhydrous Na₂SO₄, filtered, and concentrated to dryness under reduced pressure. The residue was purified by flash chromatography on silica gel (DCM:MeOH=20:1) to give compound C (87 mg, yield 83.7%) as yellow oil. LC/MS (ESI) m/z: 433 (M+H)⁺.

Step 3: (2S)-4-hydroxy-1-((4-phenoxybutanoyl)glycyl)-4-(trifluoromethyl)pyrrolidine-2-carboxylic acid (D). To a solution of compound C (87 mg, 0.2 mmol) in MeOH (1 mL), THE (0.5 mL) and H₂O (0.5 mL) was added LiOH·H₂O (8 mg, 0.2 mmol) at 0° C. and the mixture was stirred at 25° C. for 5 hours. The mixture was diluted with water and washed with EtOAc twice. The aqueous layer was acidified with 0.5 N aq. HCl to pH=3 and extracted with EtOAc twice. The combined organic layers were dried over anhydrous Na₂SO₄, filtered, and concentrated to dryness under reduced pressure to give compound D (43 mg, yield 51.2%) as a yellow oil, which was used directly in the next step without further purification. LC/MS (ESI) m/z: 419 (M+H)⁺.

Step 4: (2S)-N-((4-carbamimidoylthiophen-2-yl)methyl)-4-hydroxy-1-((4-phenoxybutanoyl)-glycyl)-4-(trifluoromethyl)pyrrolidine-2-carboxamide (Compound 62). To a mixture of compound D (43 mg, 0.1 mmol) and compound E (23 mg, 0.12 mmol) in DMF (1 mL) was added DIPEA (0.1 mL, 0.6 mmol) and T₃P (191 mg, 0.3 mmol, 50% wt in EtOAc) at 0° C. under N₂ atmosphere and the mixture was stirred at 35° C. for 16 hours. The mixture was diluted with EtOAc and washed with saturated aq. NaHCO₃ solution. The aqueous layer was extracted with CHCl₃/i-PrOH (v/v=3/1) twice and the combined organic layers were dried over anhydrous Na₂SO₄, filtered, and concentrated to dryness under reduced pressure. The residue was purified by prep-HPLC to give Compound 62 (3.5 mg, yield 6.1%) as a yellow oil. ¹H NMR (400 MHz, CD₃OD) δ 8.54 (s, 1H), 8.20 (s, 1H), 7.44 (s, 1H), 7.23 (t, J=8.0 Hz, 2H), 6.91-6.86 (m, 3H), 4.72 (d, J=8.4 Hz, 1H), 4.64-4.60 (m, 1H), 4.54-4.50 (m, 1H), 4.09 (d, J=16.8 Hz, 1H), 4.03-3.96 (m, 3H), 3.91 (dd, J=12.0, 13.6 Hz, 1H), 3.80 (d, J=10.8 Hz, 1H), 2.72-2.55 (m, 1H), 2.46 (t, J=7.4 Hz, 2H), 2.32 (d, J=13.6 Hz, 1H), 2.09-2.02 (m, 2H); LC/MS (ESI) m/z: 556 (M+H)⁺.

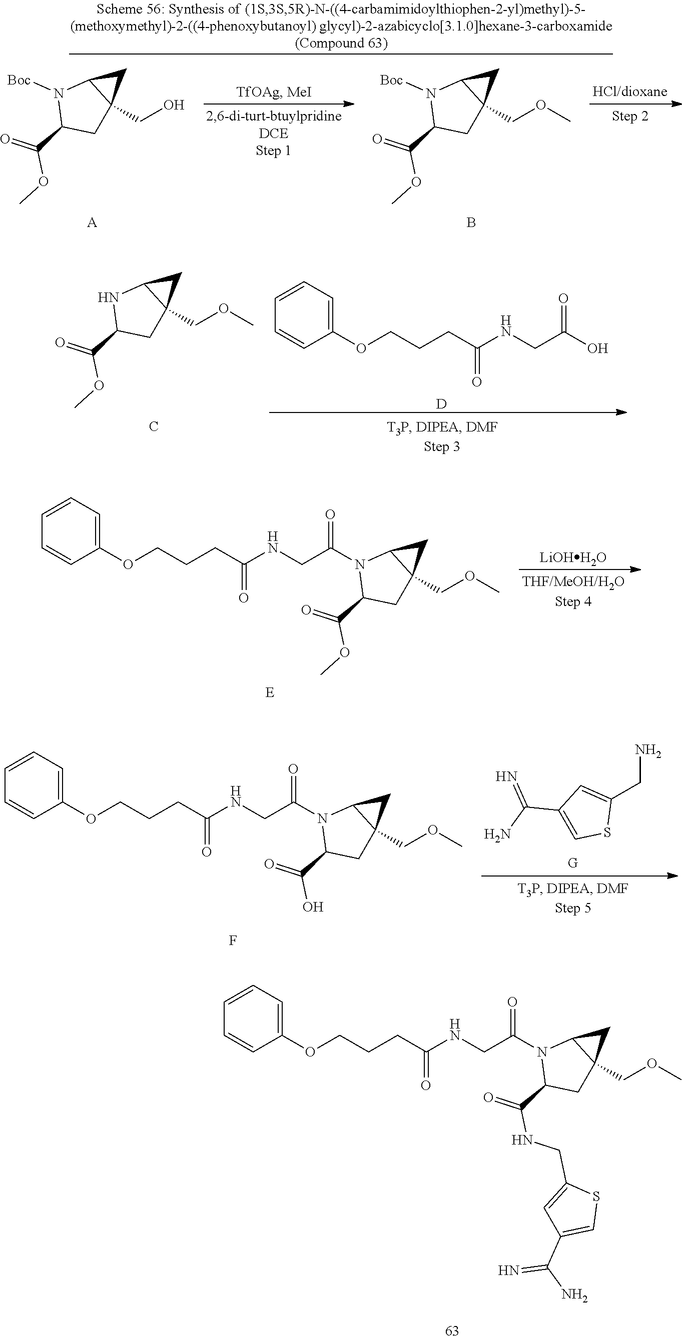

Step 1: 2-(tert-butyl) 3-methyl (1S,3S,5R)-5-(methoxymethyl)-2-azabicyclo[3.1.0]hexane-2,3-dicarboxylate (B). To a solution of compound A (100 mg, 0.36 mmol) in DCE (2 mL) was added AgOTf (142 mg, 0.54 mmol), CH₃I (156 mg, 0.11 mmol) and 2,6-di-tert-butylpyridine (0.24 mL, 0.11 mmol) at 0° C. under N₂ atmosphere and the mixture was stirred at 25° C. for 4 hours. The mixture was filtered and the filtrate was concentrated to dryness under reduced pressure. The residue was purified by flash chromatography on silica gel (PE:EtOAc=3:1) to give compound B (86 mg, yield 81.1%) as a colorless oil. LC/MS (ESI) (m/z): 286 (M+H)⁺.

Step 2: methyl (1S,3S,5R)-5-(methoxymethyl)-2-azabicyclo[3.1.0]hexane-3-carboxylate (C). A mixture of compound B (86 mg, 0.0.30 mmol) in HCl/1,4-dioxane (5 mL) was stirred at room temperature for 1 hour. The reaction mixture was concentrated to dryness under reduced pressure, co-evaporated with DCM, and dried under vacuum to give compound C (50 mg, yield 89.3%) as a yellow oil, which was used directly in the next step without further purification. LC/MS (ESI) m/z: 186 (M+H)⁺.

Step 3: methyl (1S,3S,5R)-5-(methoxymethyl)-2-((4-phenoxybutanoyl)glycyl)-2-azabicyclo-[3.1.0]hexane-3-carboxylate (E). To a mixture of compound C (50 mg, 0.27 mmol) and compound D (70 mg, 0.27 mmol) in DMF (5 mL) was added DIPEA (0.27 mL, 1.62 mmol) and T₃P (516 mg, 0.81 mmol, 50% wt. in EtOAc) at 0° C. under N₂ atmosphere and the mixture was stirred at 25° C. for 16 hours. The mixture was diluted with EtOAc and washed with saturated aq. NaHCO₃ solution. The aqueous layer was extracted with EtOAc twice and the combined organic layers were washed with brine, dried over anhydrous Na₂SO₄, filtered, and concentrated to dryness under reduced pressure. The residue was purified by flash chromatography on silica gel (DCM:MeOH=10:1) to give compound E (90 mg, yield 82.6%) as a light-yellow oil. LC/MS (ESI) (m/z): 405 (M+H)⁺.

Step 4: (1S,3S,5R)-5-(methoxymethyl)-2-((4-phenoxybutanoyl)glycyl)-2-azabicyclo[3.1.0]-hexane-3-carboxylic acid (F). To a solution of compound E (90 mg, 0.22 mmol) in MeOH (2 mL), THE (1 mL) water (1 mL) was added LiOH·H₂O (14 mg, 0.33 mmol) at 0° C. and the mixture was stirred at room temperature for 16 hours. The aqueous layer was acidified with 1N aq. HCl to pH˜3 and extracted with CHCl₃/i-PrOH (v/v=3/1) twice. The combined organic layers were dried over anhydrous Na₂SO₄, filtered, and concentrated to dryness under reduced pressure give compound F (80 mg, yield 92.0%) as a yellow solid, which was used directly in the next step without further purification. LC/MS (ESI) (m/z): 391 (M+H)⁺.

Step 5: (1S,3S,5R)-N-((4-carbamimidoylthiophen-2-yl)methyl)-5-(methoxymethyl)-2-((4-phenoxybutanoyl)glycyl)-2-azabicyclo[3.1.0]hexane-3-carboxamide (Compound 63). To a mixture of compound F (80 mg, 0.20 mmol) and compound G (48 mg, 0.30 mmol) in DMF (5 mL) was added DIPEA (0.2 mL, 1.2 mmol) and T₃P (391 mg, 0.6 mmol, 50% wt in EtOAc) at 0° C. under N₂ atmosphere and the mixture was stirred at 25° C. for 16 hours. The mixture was diluted with EtOAc and washed with saturated aq.NaHCO₃ solution. The aqueous layer was extracted with CHCl₃/i-PrOH (v/v=3/1) twice and the combined organic layers were dried over anhydrous Na₂SO₄, filtered, and concentrated to dryness under reduced pressure. The residue was purified by flash chromatography on silica gel (DCM:MeOH=5:1) and further purified by prep-HPLC to give Compound 63 (4 mg, yield 3.7%) as a white solid. ¹H NMR (400 MHz, CD₃OD) δ 8.21 (d, J=1.2 Hz, 1H), 7.40 (s, 1H), 7.27-7.21 (m, 2H), 6.90 (dd, J=7.6, 5.4 Hz, 3H), 4.81-4.79 (m, 1H), 4.50 (t, J=12.8 Hz, 2H), 4.17 (q, J=16.7 Hz, 2H), 4.00 (t, J=6.3 Hz, 2H), 3.51 (dd, J=6.1, 2.7 Hz, 1H), 3.47 (d, J=10.3 Hz, 1H), 3.39 (d, J=10.3 Hz, 1H), 3.35 (s, 3H), 2.61 (t, J=12.7 Hz, 1H), 2.47 (t, J=7.4 Hz, 2H), 2.15-2.03 (m, 3H), 1.22 (dd, J=5.8, 2.7 Hz, 1H), 1.00 (t, J=5.6 Hz, 1H); LC/MS (ESI) m/z: 528 (M+H)⁺.

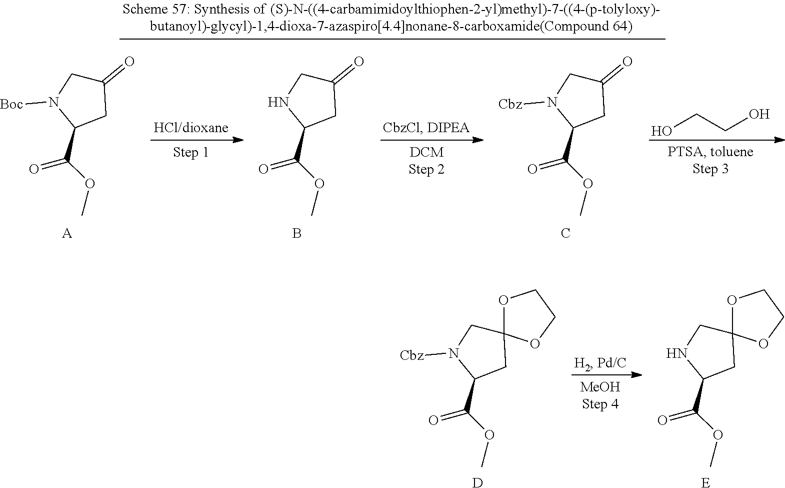

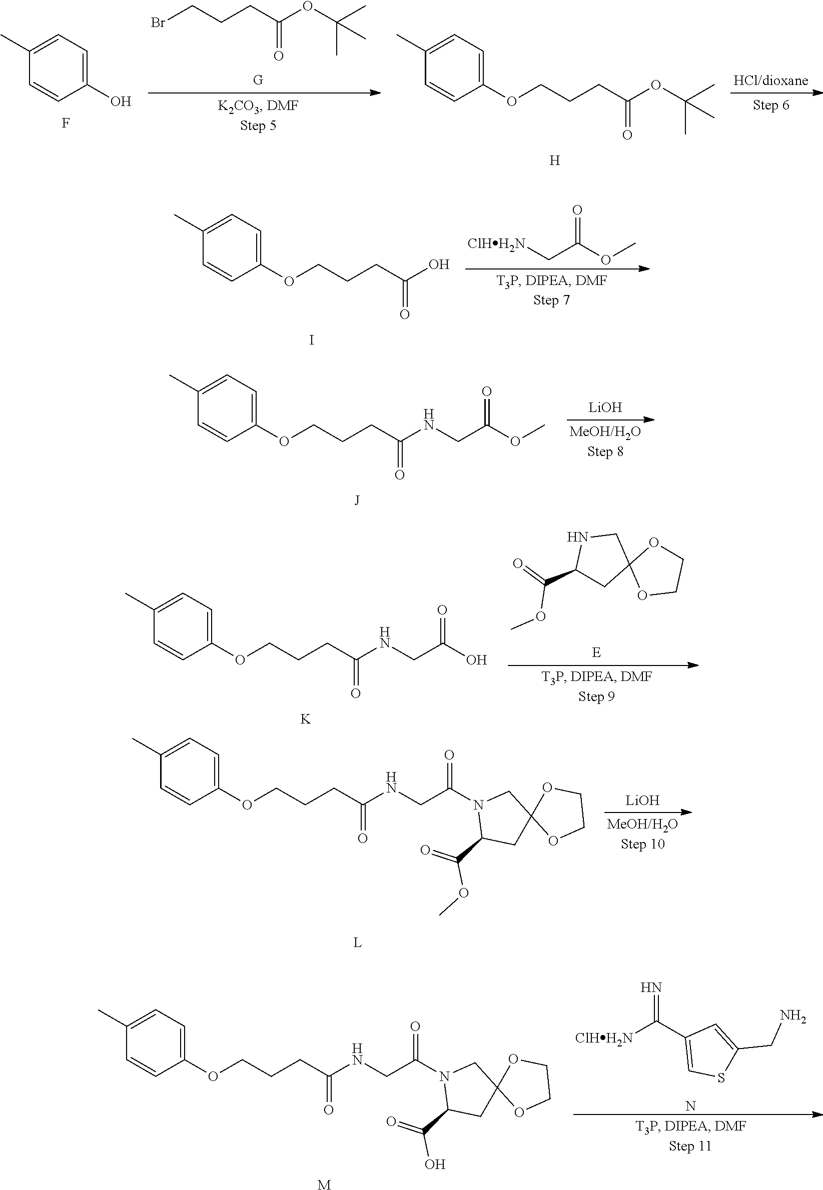

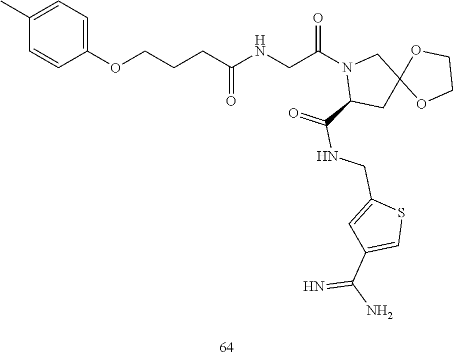 [SMILES: Cc1ccc(OCCCC(=O)NCC(=O)N2CC3(C[C@H]2C(=O)NCc2cc(C(=N)N)cs2)OCCO3)cc1 ; "64"]

Step 1: methyl (S)-4-oxopyrrolidine-2-carboxylate (B). A mixture of compound A (5.0 g, 20.55 mmol) and HCl/1,4-dioxane (20 mL) was stirred at room temperature for 2 hours. The reaction mixture was concentrated to dryness under reduced pressure, diluted with dichloromethane, and dried under vacuum to give compound B (2.94 g, quantitative yield) as a yellow solid, which was used directly in the next step without further purification. LC/MS (ESI) m/z: 144 (M+H)⁺.

Step 2: 1-benzyl 2-methyl (S)-4-oxopyrrolidine-1,2-dicarboxylate (C). To a solution of compound B (2.9 g, 20.55 mmol) and NaHCO₃ (34.4 g, 41.11 mmol) in THE (15 mL) and water (15 mL) was added benzyl chloroformate (45.58 g, 26.72 mmol) dropwise at 0° C. under N₂ atmosphere. The reaction was stirred at room temperature overnight. The mixture was extracted with ethyl acetate twice. The combined organic layers were washed with brine, dried over anhydrous Na₂SO₄, filtered and concentrated to dryness under reduced pressure. The residue was purified by column chromatography on silica gel (EtOAc:PE=1:10 to 1:3) to give compound C (4.3 g, 75.4% yield) as a colorless oil. LC/MS (ESI) m/z: 278 (M+H)⁺.

Step 3: 7-benzyl 8-methyl (S)-1,4-dioxa-7-azaspiro[4.4]nonane-7,8-dicarboxylate (D). To a solution of compound C (4.3 g, 15.51 mmol) in toluene (30 mL) was added ethane-1,2-diol (3.85 g, 62.03 mmol) and p-toluene sulfonic acid (1.48 g, 7.75 mmol) at room temperature. The reaction was stirred at 140° C. under N₂ atmosphere for 6 hours. The mixture was extracted with ethyl acetate twice. The combined organic layers were washed with brine, dried over anhydrous Na₂SO₄, filtered, and concentrated to dryness under reduced pressure. The residue was purified by column chromatography on silica gel (EtOAc:PE=1:10 to 2:3) to give compound D (3.0 g, 60.2% yield) as a light-yellow oil. LC/MS (ESI) m/z: 322(M+H)⁺.

Step 4: methyl (S)-1,4-dioxa-7-azaspiro[4.4]nonane-8-carboxylate (E). To a stirred solution of compound D (3.0 g, 9.34 mmol) in methanol (15 mL) was added Pd/C (100 mg, 10% wt.), and the mixture was degassed under H₂ atmosphere for three times and stirred under a H₂ balloon at 25° C. for 2 hours. The mixture was filtered and concentrated under reduced pressure to give crude compound E (1.7 g, 97.3% yield) as a colorless oil and used directly in the next step without further purification. LC/MS (ESI) m/z: 188 (M+H)⁺.

Step 5: tert-butyl 4-(p-tolyloxy)butanoate (H). To a solution of compound F (500 mg, 4.62 mmol) in DMF (5 mL) was added compound G (1.55 g, 6.94 mmol) and K₂CO₃ (1.60 g, 11.56 mmol) at room temperature under N₂ atmosphere. The mixture was stirred at 120° C. overnight. The reaction was cooled down to room temperature and quenched with saturated aqueous NH₄Cl. The resulting mixture was extracted with ethyl acetate (3×10 mL). The combined organic layers were washed with water (2×10 mL) and brine (2×10 mL), dried over anhydrous Na₂SO₄, filtered, and concentrated to dryness under reduced pressure. The residue was purified by column chromatography on silica gel (EtOAc/PE=1:20 to 1:10) to give compound H (720 mg, 62.2% yield) as a light-yellow oil.

Step 6: 4-(p-tolyloxy)butanoic acid (9). A mixture of compound H (220 mg, 0.879 mmol) and HCl/1,4-dioxane (3 mL) was stirred at room temperature for 1 hour. The reaction mixture was concentrated to dryness under reduced pressure, diluted with dichloromethane, and dried under vacuum to give compound I (170 mg, quantitative yield) as a white solid, which was used directly in the next step without further purification. LC/MS (ESI) m/z: 195 (M+H)⁺.

Step 7: methyl (4-(p-tolyloxy)butanoyl)glycinate (J). To a solution of compound I (170 mg, 0.875 mmol) and methyl glycinate hydrochloride (164.8 mg, 1.313 mmol) in DMF (5 mL) was added DIPEA (338.7 mg, 2.626 mmol) and T₃P (1.11 g, 1.751 mmol, 50% in ethyl acetate). The mixture was stirred at room temperature under N₂ atmosphere overnight. The reaction was quenched with saturated aq. NH₄Cl solution. The resulting mixture was extracted with ethyl acetate (3×20 mL). The combined organic layers were washed with water (2×20 mL) and brine (2×20 mL), dried over anhydrous Na₂SO₄, filtered, and concentrated to dryness under reduced pressure. The residue was purified by column chromatography on silica gel (EtOAc:PE=1:5 to 1:3) to give compound J (176 mg, 75.8% yield) as a yellow oil. LC/MS (ESI) m/z: 266 (M+H)⁺.

Step 8: (4-(p-tolyloxy)butanoyl)glycine (K). To a solution of compound J (120 mg, 0.452 mmol) in methanol (1.5 mL) and water (0.5 mL) was added a solution of LiOH·H₂O (19.0 mg, 0.452 mmol) at 0° C. and the mixture was stirred at room temperature for 2 hours. The mixture was acidified with 1 N aq. HCl solution to pH<3 and extracted with dichloromethane (2×10 mL). The combined organic layers were concentrated to dryness under reduced pressure to give compound K (100 mg, 88.0% yield) as a white solid, which was used directly in next step without further purification. LC/MS (ESI) (m/z): 252(M+H)⁺.

Step 9: methyl (S)-7-((4-(p-tolyloxy)butanoyl)glycyl)-1,4-dioxa-7-azaspiro[4.4]nonane-8-carboxylate (L). To a solution of compound K (100 mg, 0.398 mmol) and compound E (112 mg, 0.597 mmol) in DMF (3 mL) was added DIPEA (154 mg, 1.094 mmol) and T₃P (253 mg, 0.796 mmol, 50% in ethyl acetate). The mixture was stirred at room temperature under N₂ atmosphere overnight. The reaction was quenched with saturated aq. NH₄Cl solution. The resulting mixture was extracted with ethyl acetate (3×20 mL). The combined organic phases were washed with water (2×20 mL) and brine (2×20 mL), dried over anhydrous Na₂SO₄, filtered, and concentrated to dryness under reduced pressure. The residue was purified by column chromatography on silica gel (EtOAc:PE=1:5 to 1:1) to give compound L (140 mg, 83.7% yield) as a yellow oil. LC/MS (ESI) m/z: 421 (M+H)⁺.

Step 10: (S)-7-((4-(p-tolyloxy)butanoyl)glycyl)-1,4-dioxa-7-azaspiro[4.4]nonane-8-carboxylic acid (M). To a solution of compound L (140 mg, 0.333 mmol) in methanol (1.5 mL) and water (0.5 mL) was added a solution of LiOH·H₂O (14.0 mg, 0.452 mmol) at 0° C. and the mixture was stirred at room temperature for 2 hours. The mixture was acidified with 1 N aq. HCl solution to pH<3 and extracted with dichloromethane (2×10 mL). The combined organic layers were concentrated to dryness under reduced pressure to give compound M (120 mg, 88.7% yield) as a white solid, which was used directly in next step without further purification. LC/MS (ESI) (m/z): 407(M+H)⁺.

Step 11: (S)-N-((4-carbamimidoylthiophen-2-yl)methyl)-7-((4-(p-tolyloxy)butanoyl)glycyl)-1,4-dioxa-7-azaspiro[4.4]nonane-8-carboxamide (Compound 64). To a solution of compound M (60 mg, 0.148 mmol) and compound N (42.4 mg, 0.221 mmol) in DMF (3 mL) was added DIPEA (57.1 mg, 0.443 mmol) and T₃P (187.8 mg, 0.295 mmol, 50% in ethyl acetate). The mixture was stirred at room temperature under N₂ atmosphere overnight. The reaction was quenched with saturated aq. NH₄Cl solution. The resulting mixture was extracted with ethyl acetate (3×20 mL). The combined organic layers were washed with water (2×20 mL) and brine (2×20 mL), dried over anhydrous Na₂SO₄, filtered, and concentrated to dryness under reduced pressure. The residue was purified by prep-HPLC to give Compound 64 (2 mg, 2.5% yield) as a white solid. ¹H NMR (400 MHz, CD₃OD) δ 8.19 (t, J=3.1 Hz, 1H), 7.42 (d, J=27.5 Hz, 1H), 7.01 (d, J=8.2 Hz, 2H), 6.77-6.74 (m, 2H), 4.59-4.51 (m, 3H), 4.03-3.85 (m, 8H), 3.71-3.57 (m, 2H), 2.49-2.34 (m, 3H), 2.24 (d, J=17.0 Hz, 3H), 2.16 (dd, J=13.2, 5.4 Hz, 1H), 2.06-1.97 (m, 2H); LC/MS(ESI) m/z: 544 (M+H)⁺.

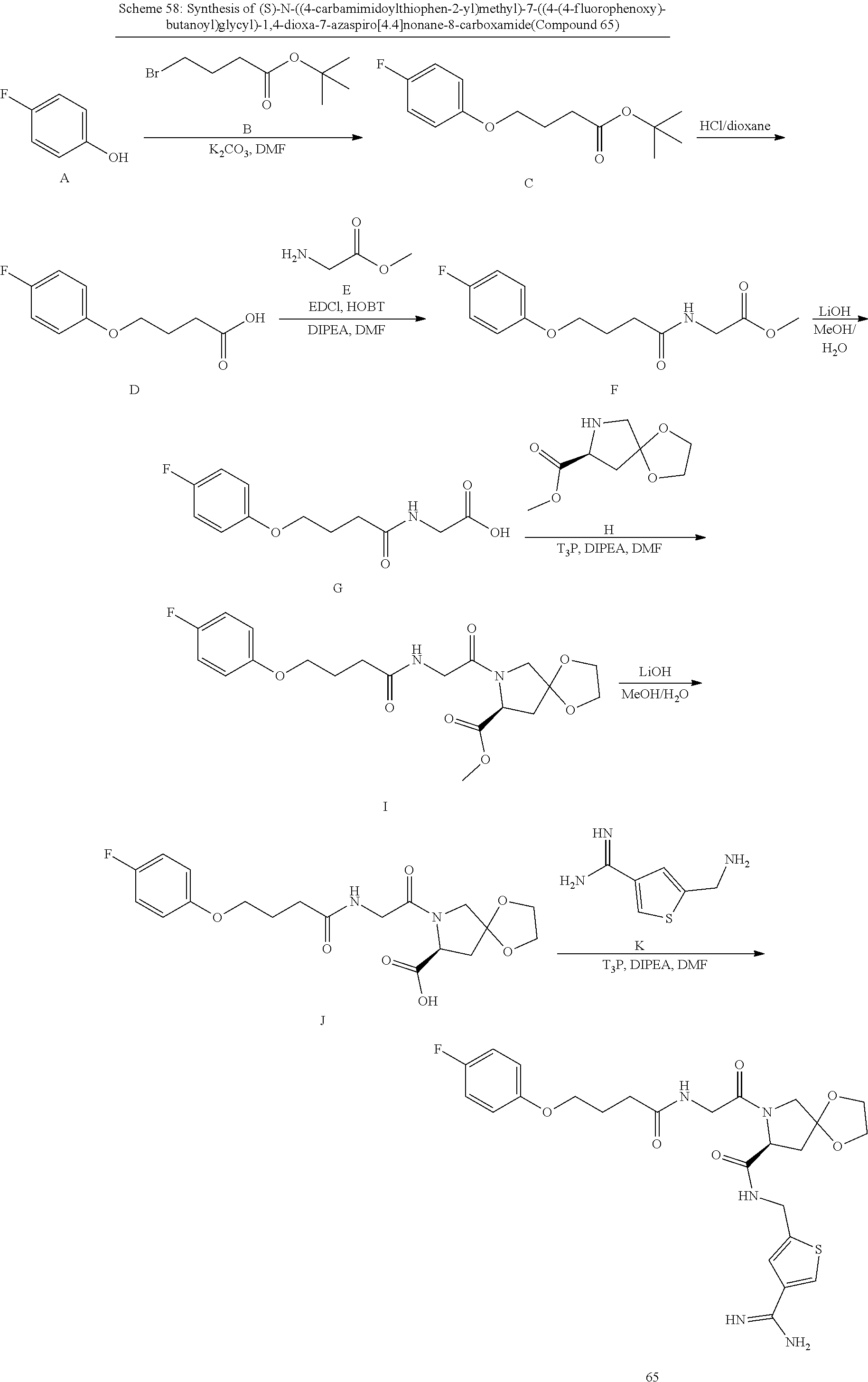

Step 1: tert-butyl 4-(4-fluorophenoxy)butanoate (C). A suspension of compound A (500 mg, 4.46 mmol), compound B (995 mg, 4.46 mmol), K₂CO₃ (1.85 g, 13.38 mmol) and KI (669 mg, 4.46 mmol) in DMF (5 mL) was heated up to 120° C. under N₂ atmosphere and stirred for 18 hours at the same temperature. The reaction suspension was partitioned between ethyl acetate and water. The organic layer was separated and washed with brine, dried over anhydrous Na₂SO₄, and concentrated to dryness under reduced pressure to give the crude product. The residue was purified by column chromatography on silica gel (petroleum ether:ethyl acetate=10:1 to 3:1) to give tert-butyl 4-(4-fluorophenoxy)butanoate (580 mg, 51.2% yield) as an oil. ¹H NMR (400 MHz, CDCl₃) δ 6.98-6.92 (m, 2H), 6.84-6.80 (m, 2H), 3.95 (t, J=6.2 Hz, 2H), 2.42 (t, J=7.3 Hz, 2H), 2.04 (td, J=7.2, 3.7 Hz, 2H), 1.45 (s, 9H).

Step 2: 4-(4-fluorophenoxy)butanoic acid (D). A mixture of compound C (240 mg, 0.94 mmol) and HCl/1,4-dioxane (2 mL) was stirred at room temperature for 3 hours. The reaction mixture was concentrated to dryness under reduced pressure, diluted with dichloromethane, and dried under vacuum to give 4-(4-fluorophenoxy)butanoic acid (170 mg, yield 91.4%), which was used directly in the next step without further purification. LC/MS (ESI) m/z: 199 (M+H)⁺.

Step 3: methyl (4-(4-fluorophenoxy)butanoyl)glycinate (F). To a mixture of 4-(4-fluorophenoxy)butanoic acid (D; 170 mg, 0.72 mmol) and compound E (136 mg, 1.08 mmol) in DMF (1 mL) was added EDCI (276 mg, 1.44 mmol), HOBT (146 mg, 1.08 mmol) and DIPEA (468 mg, 3.6 mmol) at room temperature under N₂ atmosphere. The mixture was stirred at room temperature overnight. The resulting mixture was extracted with ethyl acetate twice. The combined organic layers were washed with water and brine, dried over anhydrous Na₂SO₄, filtered, and concentrated to dryness under reduced pressure. The residue was purified by flash column chromatography on silica gel (petroleum ether:ethyl acetate=10:1 to 3:1) to give methyl (4-(4-fluorophenoxy)butanoyl)glycinate (F; 110 mg, 57.0% yield) as an oil. LC/MS (ESI) m/z: 270 (M+H)⁺.

Step 4: (4-(4-fluorophenoxy)butanoyl)glycine (G). To a solution of methyl (4-(4-fluorophenoxy)butanoyl)glycinate (F; 110 mg, 0.41 mmol) in methanol (1 mL) and water (0.25 mL) was added LiOH·H₂O (51 mg, 0.22 mmol) at 0° C. and the mixture was stirred at room temperature for 16 hours. The mixture was acidified with 1 N aq. HCl solution to pH=3 and concentrated to dryness under reduced pressure to give (4-(4-fluorophenoxy)butanoyl)glycine (G; 60 mg, 57.7% yield) as a light-yellow solid, which was used directly in next step without further purification. LC/MS (ESI) (m/z): 256 (M+H)⁺.

Step 5: methyl (S)-7-((4-(4-fluorophenoxy)butanoyl)glycyl)-1,4-dioxa-7-azaspiro[4.4]nonane -8-carboxylate (1). To a mixture of (4-(4-fluorophenoxy)butanoyl)glycine (G; 60 mg, 0.23 mmol) and compound H (65 mg, 0.35 mmol) in DMF (1 mL) was added DIPEA (89 mg, 0.69 mmol) and T₃P (222 mg, 0.35 mmol, 50% in ethyl acetate) at 0° C. under N₂ atmosphere and the mixture was stirred at 35° C. for 4 hours. The mixture was quenched with saturated aq. NaHCO₃ solution and extracted with ethyl acetate twice. The combined organic layers were washed with water and brine, dried over anhydrous Na₂SO₄, filtered, and concentrated to dryness under reduced pressure. The residue was purified by prep-HPLC to give methyl (S)-7-((4-(4-fluorophenoxy)butanoyl)glycyl)-1,4-dioxa-7-azaspiro[4.4]nonane-8-carboxylate (I; 40 mg, 40.0% yield) as an oil. LC/MS (ESI) (m/z): 425 (M+H)⁺.

Step 6: (S)-7-((4-(4-fluorophenoxy)butanoyl)glycyl)-1,4-dioxa-7-azaspiro[4.4]nonane-8-carboxylic acid (J). To a solution of methyl (S)-7-((4-(4-fluorophenoxy)butanoyl)glycyl)-1,4-dioxa-7-azaspiro[4.4]nonane-8-carboxylate (1; 40 mg, 0.094 mmol) in methanol (1 mL) and water (0.25 mL) was added LiOH·H₂O (12 mg, 0.28 mmol) at 0° C., and the mixture was stirred at room temperature for 16 hours. The mixture was acidified with 1 N aq. HCl solution to pH=3 and concentrated to dryness under reduced pressure to give (S)-7-((4-(4-fluorophenoxy)butanoyl)glycyl)-1,4-dioxa-7-azaspiro[4.4]nonane-8-carboxylic acid (J; 30 mg, 77.8% yield) as a light-yellow solid, which was used directly in next step without further purification. LC/MS (ESI) (m/z): 410 (M+H)⁺.

Step 7: (S)-N-((4-carbamimidoylthiophen-2-yl)methyl)-7-((4-(4-fluorophenoxy)butanoyl) glycyl)-1,4-dioxa-7-azaspiro[4.4]nonane-8-carboxamide (Compound 65). To a mixture of (S)-7-((4-(4-fluorophenoxy)butanoyl)glycyl)-1,4-dioxa-7-azaspiro[4.4]nonane-8-carboxylic acid (J; 30 mg, 0.073 mmol) and compound K (21 mg, 0.11 mmol) in DMF (1 mL) was added DIPEA (37 mg, 0.26 mmol) and T₃P (70 mg, 0.11 mmol, 50% in ethyl acetate) at 0° C. under N₂ atmosphere, and the mixture was stirred at 35° C. for 18 hours. The mixture was quenched with saturated aq.NaHCO₃ solution and extracted with ethyl acetate twice. The combined organic layers were washed with water and brine, dried over anhydrous Na₂SO₄, filtered, and concentrated to dryness under reduced pressure. The residue was further purified by prep-HPLC to give the Compound 65 (1.1 mg, 2.7% yield) as a white solid. ¹H NMR (400 MHz, CD₃OD) δ 8.22 (t, J=3.4 Hz, 1H), 7.45 (d, J=26.7 Hz, 1H), 6.97 (t, J=8.8 Hz, 2H), 6.91-6.86 (m, 2H), 4.62 (s, 2H), 4.55 (d, J=4.1 Hz, 2H), 4.06-3.96 (m, 6H), 3.94 (d, J=12.0 Hz, 2H), 3.70 (d, J=2.9 Hz, 2H), 2.45 (t, J=7.4 Hz, 2H), 2.21-2.17 (m, 1H), 2.04 (d, J=6.2 Hz, 2H); LC/MS (ESI) m/z: 548 (M+H)⁺.

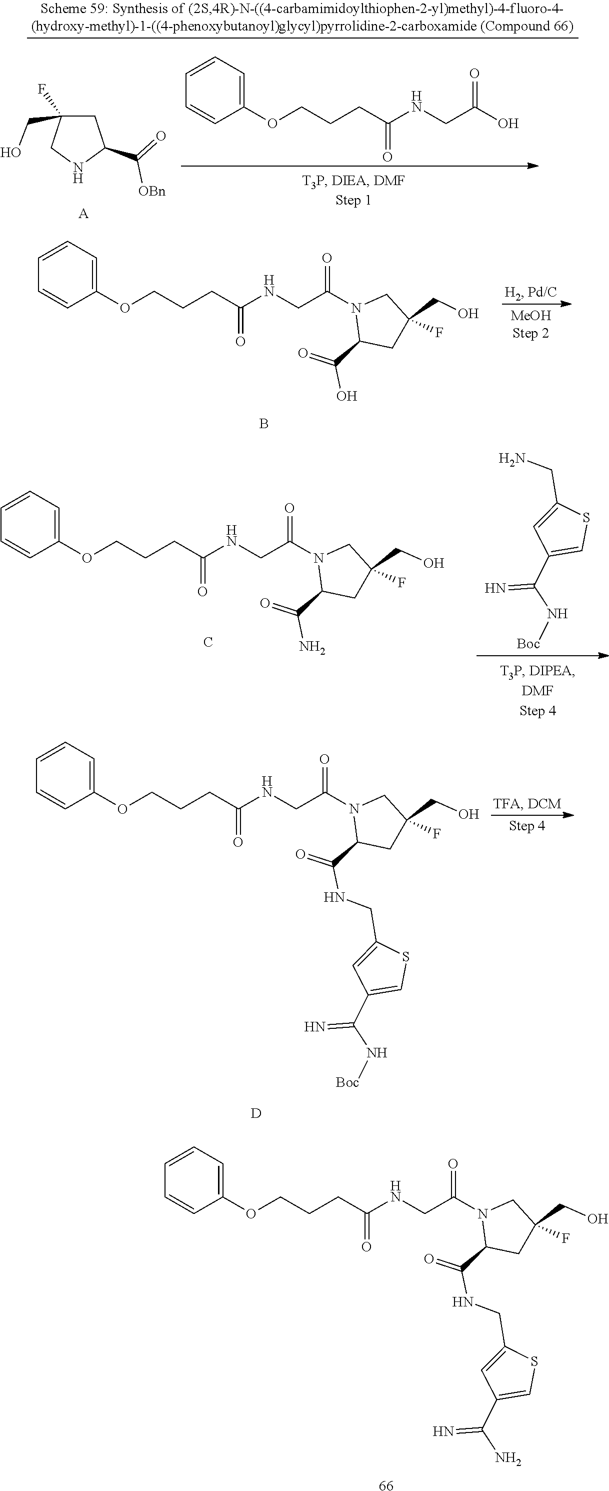

Step 1: benzyl (2S,4R)-4-fluoro-4-(hydroxymethyl)-1-((4-phenoxybutanoyl)glycyl)-pyrrolidine-2-carboxylate (B). To a mixture of compound A (210 mg, 0.83 mmol) and (4-phenoxybutanoyl)glycine (197 mg, 0.83 mmol) in DMF (5 mL) was added DIPEA (0.87 mL, 4.98 mmol) and T₃P (1.58 g, 2.49 mmol, 50% wt. in EtOAc) at 0° C. and the mixture was stirred under N₂ atmosphere at 25° C. for 3 hours. The mixture was diluted with EtOAc and washed with saturated aq. NaHCO₃ solution. The aqueous layer was extracted with EtOAc twice and the combined organic layers were washed with brine, dried over anhydrous Na₂SO₄, filtered, and concentrated to dryness under reduced pressure. The residue was purified by flash chromatography on silica gel (DCM:MeOH=10:1) to give compound B (380 mg, 97.0% yield) as an oil. LC/MS (ESI) m/z: 473 (M+H)⁺.

Step 2: (2S,4R)-4-fluoro-4-(hydroxymethyl)-1-((4-phenoxybutanoyl)glycyl)pyrrolidine-2-carboxylic acid (C). To a stirred solution of compound B (380 mg, 0.80 mmol) in MeOH (4 mL) was added Pd/C (100 mg, 10% wt.) under N₂ atmosphere at room temperature and the reaction was stirred under H₂ atmosphere at room temperature for 2 hours. The mixture was filtered and concentrated to dryness under reduced pressure to give compound C (290 mg, yield 94.3%) as a colorless oil, which was used directly in the next step without further purification. LC/MS (ESI) m/z: 383 (M+H)⁺.

Step 3: tert-butyl ((5-(((2S,4R)-4-fluoro-4-(hydroxymethyl)-1-((4-phenoxybutanoyl)glycyl)-pyrrolidine-2-carboxamido)methyl)thiophen-3-yl)(imino)methyl)carbamate (D). To a mixture of compound C (290 mg, 0.76 mmol) and tert-butyl ((5-(aminomethyl)thiophen-3-yl)(imino)methyl)carbamate (194 mg, 0.76 mmol) in DMF (6 mL) was added DIPEA (0.80 mL, 4.55 mmol) and T₃P (1.45 g, 2.28 mmol, 50% wt in EtOAc) at 0° C. and the mixture was stirred under N₂ atmosphere at 25° C. for 16 hours. The mixture was diluted with EtOAc and washed with saturated aq. NaHCO₃ solution. The aqueous layer was extracted with EtOAc twice and the combined organic layers were washed with brine, dried over anhydrous Na₂SO₄, filtered, and concentrated to dryness under reduced pressure. The residue was purified by flash chromatography on silica gel (DCM:MeOH=15:1) to give compound D (260 mg, 55.3% yield) as a white solid. LC/MS (ESI) m/z: 620 (M+H)⁺.

Step 4: (2S,4R)-N-((4-carbamimidoylthiophen-2-yl)methyl)-4-fluoro-4-(hydroxymethyl)-1-((4-phenoxybutanoyl)glycyl)pyrrolidine-2-carboxamide (Compound 66). To a solution of compound D (50 mg, 0.08 mmol) in DCM (2 mL) was added TFA (1 mL) at 0° C. and the reaction was stirred at room temperature for 1 hour. The reaction mixture was concentrated to dryness under reduced pressure, co-evaporated with DCM twice and dried under vacuum. The residue was purified by prep-HPLC to give Compound 66 (5 mg, 12.9% yield) as a white solid. ¹H NMR (400 MHz, CD₃OD) δ 8.53 (s, 1H), 8.26-8.19 (m, 1H), 7.46 (s, 1H), 7.23 (t, J=8.0 Hz, 2H), 6.93-6.84 (m, 3H), 4.65-4.53 (m, 3H), 4.11-3.72 (m, 8H), 2.58-2.41 (m, 3H), 2.20-2.01 (m, 3H); LC/MS (ESI) m/z: 520 (M+H)⁺.

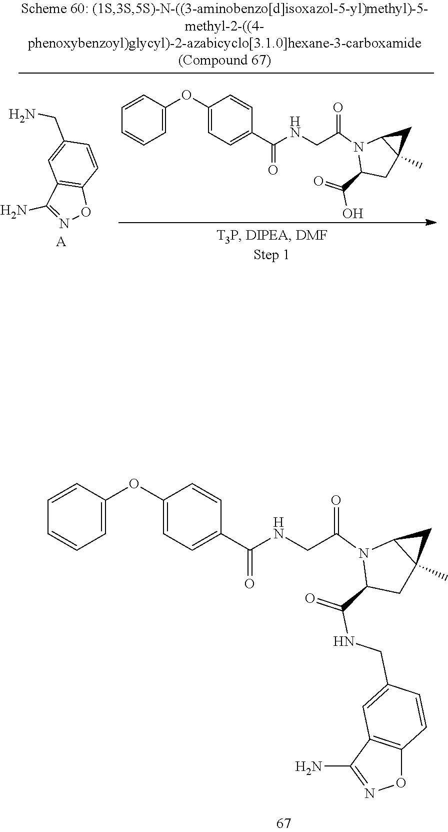

Step 1: (1S,3S,5S)-N-((3-aminobenzo[d]isoxazol-5-yl)methyl)-5-methyl-2-((4-phenoxy-benzoyl)glycyl)-2-azabicyclo[3.1.0]hexane-3-carboxamide (Compound 67). To a mixture of compound A (50 mg, 0.31 mmol) and (1S,3S,5S)-5-methyl-2-((4-phenoxybenzoyl)glycyl)-2-azabicyclo[3.1.0]hexane-3-carboxylic acid (122 mg, 0.31 mmol) in DMF (1 mL) was added DIPEA (0.3 mL, 0.92 mmol) and T₃P (585 mg, 0.92 mmol, 50% wt in EtOAc) at 0° C. and the mixture was stirred under N₂ atmosphere at 30° C. for 16 hours. The mixture was diluted with EtOAc and washed with saturated aq. NaHCO₃ solution. The aqueous layer was extracted with EtOAc twice and the combined organic layers were washed with brine, dried over anhydrous Na₂SO₄, filtered, and concentrated to dryness under reduced pressure. The residue was purified by prep-HPLC to give Compound 67 (15 mg, yield 9.0%) as a white solid. ¹H NMR (400 MHz, CD₃OD) δ 7.79-7.74 (m, 2H), 7.66 (d, J=0.8 Hz, 1H), 7.46-7.39 (m, 3H), 7.28 (d, J=12.8 Hz, 1H), 7.20 (t, J=7.4 Hz, 1H), 7.08-7.04 (m, 2H), 6.97-6.93 (m, 2H), 4.88 (d, J=3.2 Hz, 1H), 4.54-4.40 (m, 2H), 4.39-4.25 (m, 2H), 3.39 (dd, J=6.0, 6.0 Hz, 1H), 2.46-2.36 (m, 1H), 2.19 (dd, J=12.8, 13.6 Hz, 1H), 1.29 (s, 3H), 1.20-1.14 (m, 1H), 0.79 (t, J=5.4 Hz, 1H); LC/MS (ESI) m/z: 540 (M+H)⁺.

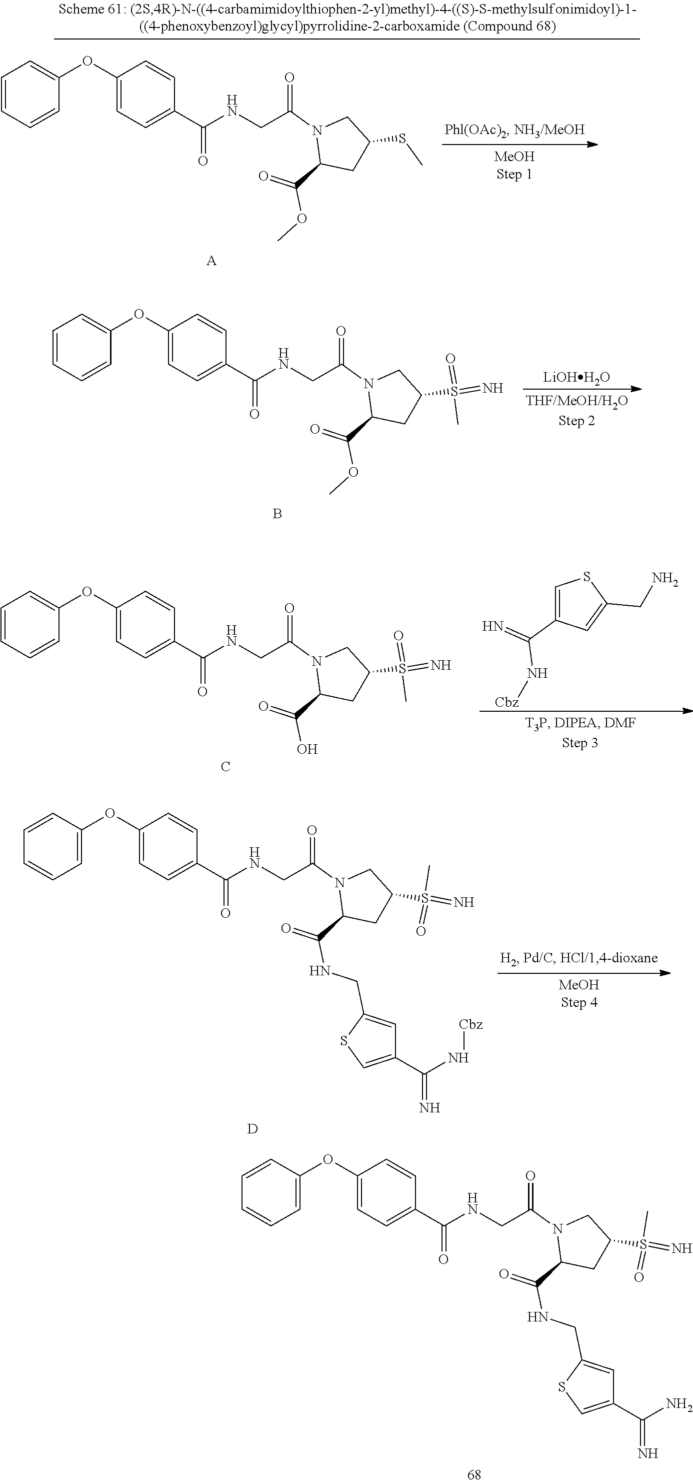

Step 1: methyl(2S,4R)-4-((S)-S-methylsulfonimidoyl)-1-((4-phenoxybenzoyl)glycyl)pyrroli dine-2-carboxylate (B). To a solution of compound A (306 mg, 0.71 mmol) in MeOH (3 mL) was added NH₃/MeOH (0.15 mL, 1.07 mmol, 7 M) and Phl(OAC)₂ (530 mg, 1.64 mmol) at 0° C. The reaction was stirred at room temperature for 40 minutes and the mixture was concentrated to dryness under reduced pressure. The residue was purified by flash chromatography on silica gel (DCM:MeOH=10:1) to give compound B (180 mg, yield 55.4%) as a yellow oil. LC/MS (ESI) m/z: 460 (M+H)⁺.

Step 2: (2S,4R)-4-((S)-S-methylsulfonimidoyl)-1-((4-phenoxybenzoyl)glycyl)pyrrolidine-2-carboxylic acid (C). To a solution of compound B (180 mg, 0.39 mmol) in MeOH (2 mL), THE (1 mL) and H₂O (1 mL) was added LiOH·H₂O (16 mg, 0.39 mmol) at 0° C., and the mixture was stirred at 25° C. for 3 hours. The mixture was diluted with water and washed with EtOAc twice. The aqueous layer was acidified with 0.5 M aq. HCl solution and extracted with EtOAc twice. The combined organic layers were dried over anhydrous Na₂SO₄, filtered, and concentrated to dryness under reduced pressure to give compound 3 (132 mg, yield 76.3%) as a white solid, which was used directly in the next step without further purification. LC/MS (ESI) m/z: 446 (M+H)⁺.

Step 3: benzyl (imino(5-(((2S,4R)-4-((S)-S-methylsulfonimidoyl)-1-((4-phenoxybenzoyl)-glycyl)pyrrolidine-2-carboxamido)methyl)thiophen-3-yl)methyl)carbamate (D). To a mixture of compound C (60 mg, 0.13 mmol) and benzyl ((5-(aminomethyl)thiophen-3-yl)(imino)methyl)carbamate (53 mg, 0.18 mmol) in DMF (1 mL) was added DIPEA (0.1 mL, 0.78 mmol) and T₃P (248 mg, 0.39 mmol, 50% wt in EtOAc) at 0° C., and the mixture was stirred under N₂ atmosphere at 30° C. for 16 hours. The mixture was diluted with EtOAc and washed with saturated aq. NaHCO₃ solution. The aqueous layer was extracted with EtOAc twice and the combined organic layers were washed with brine, dried over anhydrous Na₂SO₄, filtered, and concentrated to dryness under reduced pressure. The residue was purified by flash chromatography on silica gel (DCM:MeOH=10:1) to give compound D (73 mg, yield 78.5%) as a yellow oil. LC/MS (ESI) m/z: 717 (M+H)⁺.

Step 4: (2S,4R)-N-((4-carbamimidoylthiophen-2-yl)methyl)-4-((S)-S-methylsulfonimidoyl)-1-((4-phenoxybenzoyl)glycyl)pyrrolidine-2-carboxamide (Compound 68).

To a solution of compound D (73 mg, 0.1 mmol) in MeOH (2 mL) was added Pd/C (26 mg, 10% wt.) and HCl/1,4-dioxane (2 drops) under N₂ atmosphere at room temperature, and the reaction mixture was stirred under a H₂ balloon at room temperature for 16 hours. The mixture was filtered, and the filtrate was concentrated to dryness under reduced pressure. The residue was purified by prep-HPLC to give Compound 68 (11 mg, yield 20.0%) as a white solid. ¹H NMR (400 MHz, CD₃OD) δ 8.52 (s, 1H), 8.23 (s, 1H), 7.84 (d, J=8.8 Hz, 2H), 7.43-7.39 (m, 3H), 7.20 (t, J=7.4 Hz, 1H), 7.06 (d, J=8.4 Hz, 2H), 7.00 (dd, J=8.4, 8.8 Hz, 2H), 4.79-4.68 (m, 1H), 4.66-4.54 (m, 2H), 4.30-3.95 (m, 5H), 3.07 (s, 3H), 2.88-2.66 (m, 1H), 2.60-2.39 (m, 1H); LC/MS (ESI) m/z: 583 (M+H)⁺.

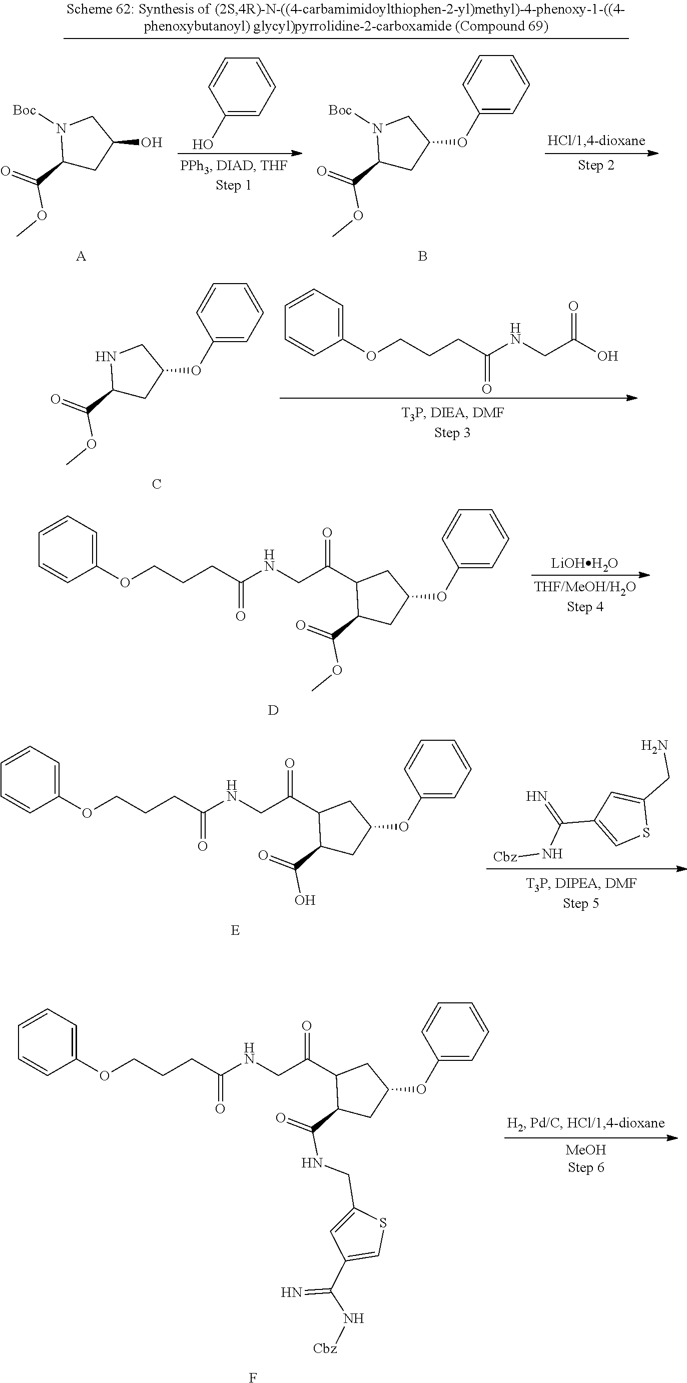

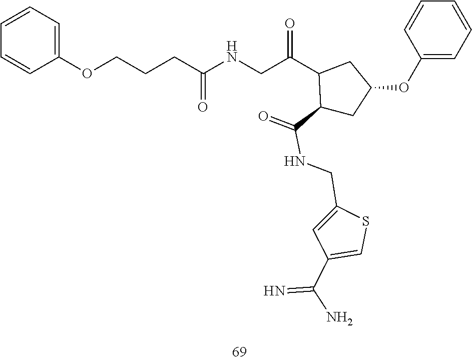 [SMILES: N=C(N)c1csc(CNC(=O)[C@@H]2C[C@@H](Oc3ccccc3)CC2C(=O)CNC(=O)CCCOc2ccccc2)c1 ; "69"]

Step 1: 1-(tert-butyl) 2-methyl (2S,4R)-4-phenoxypyrrolidine-1,2-dicarboxylate (B). PPh₃ (1.6 g, 6.12 mmol) and phenol (576 mg, 6.12 mmol) were added to a solution of compound A (1.0 g, 4.0 mmol) in THE (15 mL) under N₂ atmosphere at 0° C. DIAD (3.2 mL, 6.12 mmol) was then added slowly to the reaction mixture at 0° C. and the mixture was stirred at 25° C. for 16 hours. The mixture was diluted with EtOAc and washed with brine. The aqueous layer was extracted with EtOAc twice and the combined organic layers were dried over anhydrous Na₂SO₄, filtered, and concentrated to dryness under reduced pressure. The residue was purified by flash chromatography on silica gel (PE:EtOAc=20:1) to give compound B (845 mg, yield 65.0%) as a colorless oil. LC/MS (ESI) m/z: 322 (M+H)⁺.

Step 2: methyl (2S,4R)-4-phenoxypyrrolidine-2-carboxylate (C). A mixture of compound B (500 mg, 1.55 mmol) and HCl/1,4-dioxane (10 mL, 4M) was stirred at room temperature for 1 hour. The reaction mixture was concentrated to dryness under reduced pressure, co-evaporated with DCM twice, and dried under vacuum to give compound C (343 mg, yield 100.0%) as a colorless oil, which was used directly in the next step without further purification. LC/MS (ESI) m/z: 222 (M+H)⁺.

Step 3: methyl (2S,4R)-4-phenoxy-1-((4-phenoxybutanoyl)glycyl)pyrrolidine-2-carboxylate (D). To a mixture of compound C (170 mg, 0.77 mmol) and (4-phenoxybutanoyl)glycine (184 mg, 0.77 mmol) in DMF (5 mL) was added DIPEA (0.76 mL, 4.62 mmol) and T₃P (1.5 g, 2.31 mmol, 50% wt. in EtOAc) at 0° C., and the mixture was stirred under N₂ atmosphere at 25° C. for 16 hours. The mixture was diluted with EtOAc and washed with saturated aq. NaHCO₃ solution. The aqueous layer was extracted with EtOAc twice and the combined organic layers were washed with brine, dried over anhydrous Na₂SO₄, filtered, and concentrated to dryness under reduced pressure. The residue was purified by flash chromatography on silica gel (DCM:MeOH=30:1) to give compound D (330 mg, yield 97.6%) as a yellow solid. LC/MS (ESI) m/z: 441 (M+H)⁺.

Step 4: (2S,4R)-4-phenoxy-1-((4-phenoxybutanoyl)glycyl)pyrrolidine-2-carboxylic acid (E). To a solution of compound D (330 mg, 0.75 mmol) in MeOH (2 mL), THE (1 mL) and H₂O (1 mL) was added LiOH·H₂O (47 mg, 1.12 mmol) at 0° C. and the mixture was stirred at room temperature for 2 hours. The aqueous layer was acidified with 1 N aq. HCl solution to pH˜3 and extracted with EtOAc twice. The combined organic layers were washed with brine, dried over anhydrous Na₂SO₄, filtered, and concentrated to dryness under reduced pressure to give compound D (300 mg, yield 93.75%) as a white solid, which was used directly in next step without further purification. LC/MS (ESI) (m/z): 427 (M+H)⁺.

Step 5: benzyl (imino(5-(((2S,4R)-4-phenoxy-1-((4-phenoxybutanoyl)glycyl)pyrrolidine-2-carboxamido)methyl)thiophen-3-yl)methyl)carbamate (E). To a mixture of compound D (100 mg, 0.23 mmol) and benzyl ((5-(aminomethyl)thiophen-3-yl)(imino)methyl)carbamate (55 mg, 0.35 mmol) in DMF (5 mL) was added DIPEA (0.23 mL, 2.1 mmol) and T₃P (150 mg, 1.05 mmol, 50% wt in EtOAc) at 0° C., and the mixture was stirred under N₂ atmosphere at 25° C. for 16 hours. The mixture was diluted with EtOAc and washed with saturated aq. NaHCO₃ solution. The aqueous layer was extracted with EtOAc twice and the combined organic layers were washed with brine, dried over anhydrous Na₂SO₄, filtered, and concentrated to dryness under reduced pressure. The residue was purified by flash chromatography on silica gel (DCM:MeOH=3:1) to give compound E (80 mg, yield 49.1%) as a white solid. LC/MS (ESI) m/z: 698 (M+H)⁺.

Step 6: (2S,4R)-N-((4-carbamimidoylthiophen-2-yl)methyl)-4-phenoxy-1-((4-phenoxy-butanoyl)glycyl)pyrrolidine-2-carboxamide (Compound 69). To a solution of compound E (80 mg, 0.11 mmol) in MeOH (5 mL) was added Pd/C (20 mg, 10% wt) and HCl/1,4-dioxane (2 drops) under N₂ atmosphere at room temperature, and the reaction mixture was stirred under a H₂ balloon at room temperature for 16 hours. The mixture was filtered and the filtrate was concentrated to dryness under reduced pressure. The residue was purified by prep-HPLC to give Compound 69 (10 mg, yield 15.4%) as a white solid. ¹H NMR (400 MHz, CD₃OD) δ 8.53 (s, 1H), 8.22 (s, 1H), 7.46 (s, 1H), 7.33-7.19 (m, 4H), 7.00-6.85 (m, 6H), 5.09 (s, 1H), 4.63-4.49 (m, 3H), 4.08-3.74 (m, 6H), 2.56-2.40 (m, 3H), 2.21 (ddd, J=13.3, 8.5, 4.6 Hz, 1H), 2.10-2.01 (m, 2H); LC/MS (ESI) m/z: 564 (M+H)⁺.

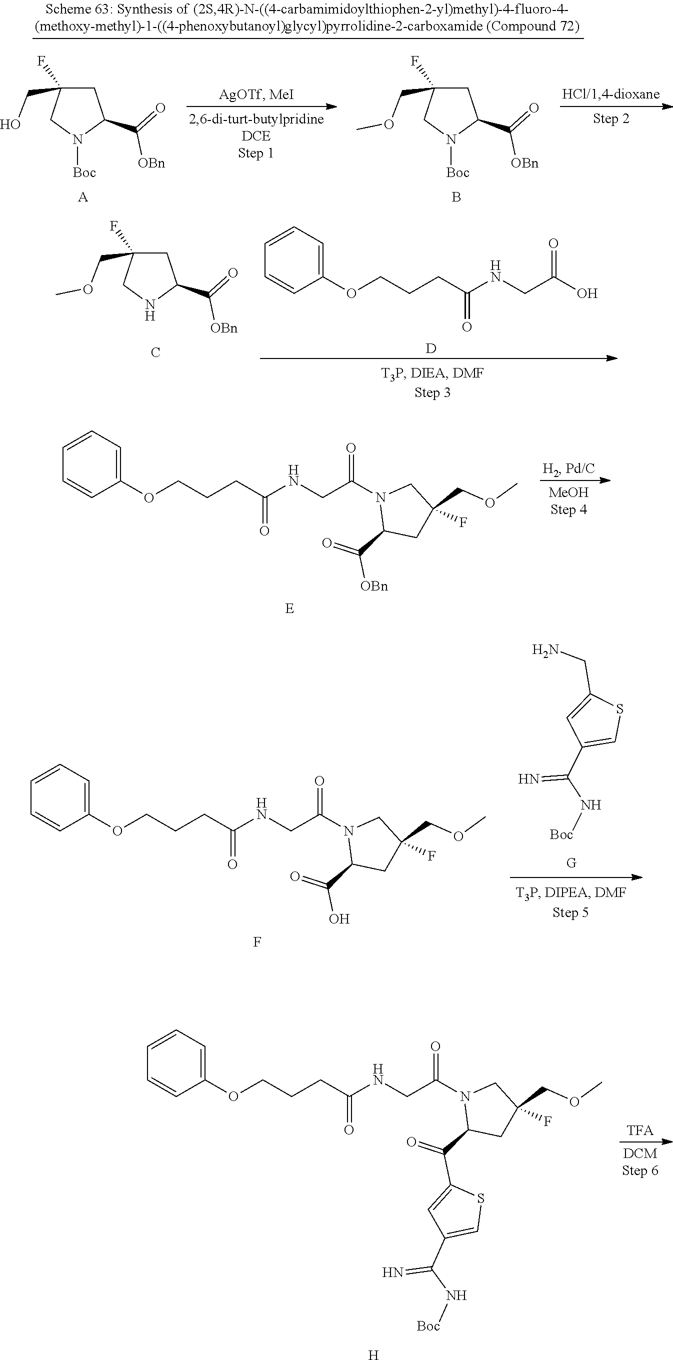

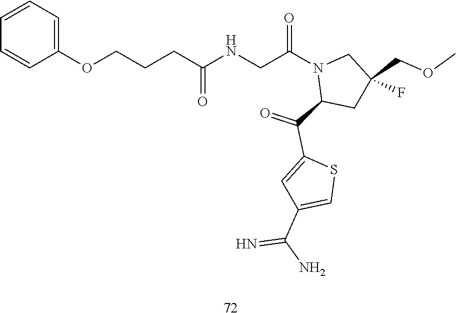 [SMILES: COC[C@@]1(F)C[C@@H](C(=O)c2cc(C(=N)N)cs2)N(C(=O)CNC(=O)CCCOc2ccccc2)C1 ; "72"]

Step 1: 2-benzyl 1-(tert-butyl) (2S,4R)-4-fluoro-4-(methoxymethyl)pyrrolidine-1,2-dicarboxylate (B). To a solution of compound A (350 mg, 0.99 mmol) in DCE (6 mL) was added AgOTf (382 mg, 1.49 mmol), CH₃I (422 mg, 2.97 mmol), and 2,6-di-tert-butylpyridine (0.67 mL, 2.97 mmol) at 0° C. and the mixture was stirred under N₂ atmosphere at 25° C. for 16 hours. The mixture was filtered, and the filtrate was concentrated to dryness under reduced pressure. The residue was purified by flash chromatography on silica gel (PE:EtOAc=3:1) to give compound B (60 mg, yield 16.5%) as a colorless oil. LC/MS (ESI) (m/z): 368 (M+H)⁺.

Step 2: benzyl (2S,4R)-4-fluoro-4-(methoxymethyl)pyrrolidine-2-carboxylate (C). A mixture of compound B (60 mg, 0.16 mmol) in HCl/1,4-dioxane (2 mL) was stirred at room temperature for 1.5 hours. The reaction mixture was concentrated to dryness under reduced pressure, co-evaporated with DCM twice, and dried under vacuum to give compound C (43 mg, yield 98.5%) as a white solid, which was used directly in the next step. LC/MS (ESI) m/z: 268 (M+H)⁺.

Step 3: benzyl (2S,4R)-4-fluoro-4-(methoxymethyl)-1-((4-phenoxybutanoyl)glycyl)-pyrrolidine-2-carboxylate (E). To a mixture of compound C (43 mg, 0.16 mmol) and (4-phenoxybutanoyl)glycine (D; 37 mg, 0.16 mmol) in DMF (3 mL) was added DIPEA (0.17 mL, 0.94 mmol), and T₃P (300 mg, 0.47 mmol, 50% wt in EtOAc) at 0° C., and the mixture was stirred under N₂ atmosphere at 25° C. for 16 hours. The mixture was diluted with EtOAc and washed with saturated aq. NaHCO₃ solution and the aqueous layer was extracted with EtOAc twice. The combined organic layers were washed with brine, dried over anhydrous Na₂SO₄, filtered, and concentrated to dryness under reduced pressure. The residue was purified by flash chromatography on silica gel (PE:EtOAc=1:2) to give compound E (53 mg, yield 69.3%) as a light-yellow oil. LC/MS (ESI) (m/z): 487 (M+H)⁺.

Step 4: (2S,4R)-4-fluoro-4-(methoxymethyl)-1-((4-phenoxybutanoyl)glycyl)pyrrolidine-2-carboxylic acid (F). To a solution of compound E (50 mg, 0.10 mmol) in MeOH (3 mL) was added Pd/C (15 mg, 10% wt.) at room temperature under N₂ atmosphere, and the mixture was stirred under a H₂ balloon at room temperature for 5 hours. The mixture was filtered, and the filtrate was concentrated to dryness under reduced pressure to give compound F (40 mg, yield 98.3%) as a colorless oil, which was used directly in the next step. LC/MS (ESI) (m/z): 397 (M+H)⁺.

Step 5: tert-butyl ((5-(((2S,4R)-4-fluoro-4-(methoxymethyl)-1-((4-phenoxybutanoyl)-glycyl)pyrrolidine-2-carboxamido)methyl)thiophen-3-yl)(imino)methyl)carbamate (H). To a mixture of compound G (40 mg, 0.10 mmol) and tert-butyl ((5-(aminomethyl)thiophen-3-yl)(imino)methyl)carbamate (H; 26 mg, 0.10 mmol) in DMF (3 mL) was added DIPEA (0.1 mL, 0.6 mmol) and T₃P (191 mg, 0.30 mmol, 50% wt in EtOAc) at 0° C., and the mixture was stirred under N₂ atmosphere at 25° C. for 2 hours. The mixture was diluted with EtOAc and washed with saturated aq. NaHCO₃ solution. The aqueous layer was extracted with EtOAc twice. The combined organic layers were washed with brine, dried over anhydrous Na₂SO₄, filtered, and concentrated to dryness under reduced pressure. The residue was purified by flash chromatography on silica gel (DCM:MeOH=10:1) to give compound H (45 mg, yield 71.1%) as a white solid. LC/MS (ESI) (m/z): 634 (M+H)⁺.

Step 6: (2S,4R)-N-((4-carbamimidoylthiophen-2-yl)methyl)-4-fluoro-4-(methoxymethyl)-1-((4-phenoxybutanoyl)glycyl)pyrrolidine-2-carboxamide (Compound 72). To a solution of compound 8 (45 mg, 0.07 mmol) in DCM (2 mL) was added TFA (1 mL) at 0° C. and, the reaction mixture was stirred at room temperature for 1.5 hours. The reaction mixture was concentrated to dryness under reduced pressure, co-evaporated with DCM twice, and dried under vacuum. The residue was purified by prep-HPLC to give Compound 72 (2.3 mg, yield 6.1%) as a white solid. ¹H NMR (400 MHz, CD₃OD) δ 8.55 (s, 1H), 8.24 (dd, J=5.2, 5.2 Hz, 1H), 7.46 (s, 1H), 7.28-7.21 (m, 2H), 6.95-6.85 (m, 3H), 4.65-4.51 (m, 3H), 4.11-3.72 (m, 6H), 3.70-3.57 (m, 2H), 3.41 (s, 3H), 2.65-2.37 (m, 3H), 2.26-1.97 (m, 3H); LC/MS (ESI) (m/z): 534 (M+H)⁺.

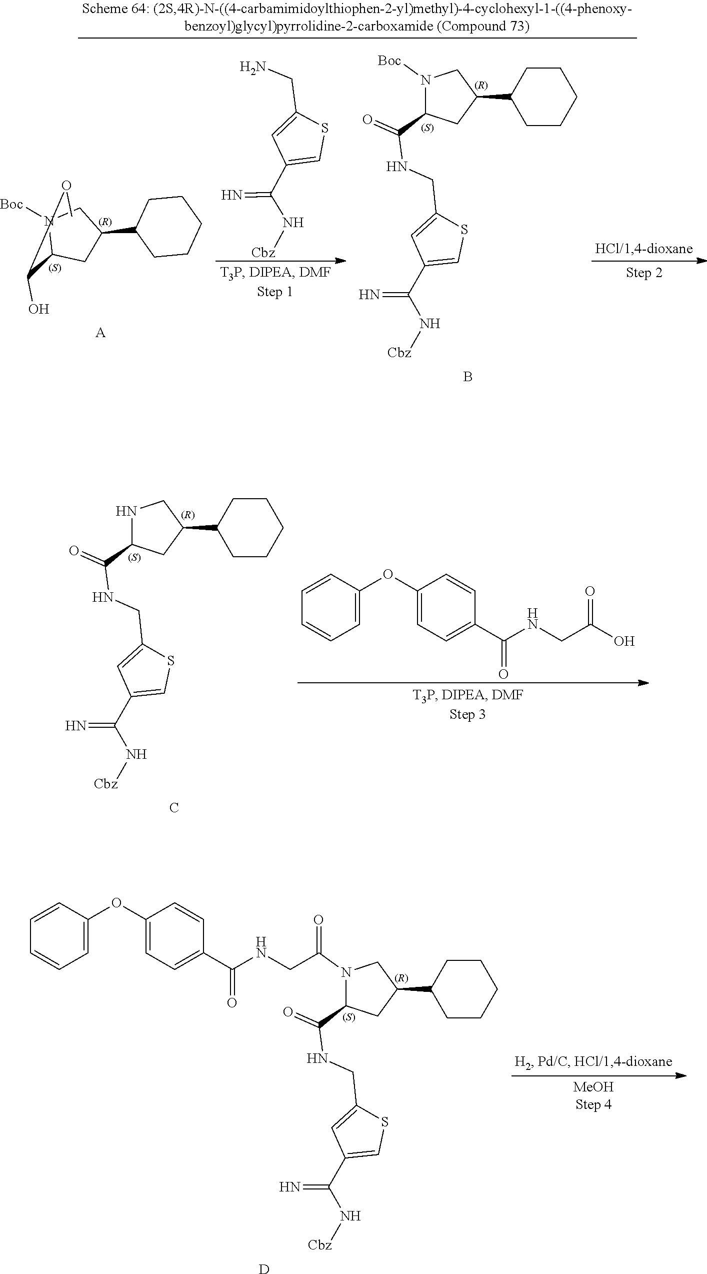

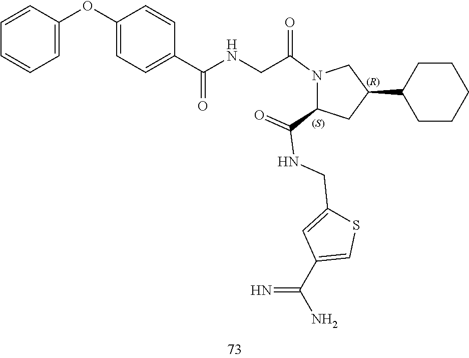

Step 1: tert-butyl (2S,4R)-2-(((4-(N-((benzyloxy)carbonyl)carbamimidoyl)thiophen-2-yl)methyl)carbamoyl)-4-cyclohexylpyrrolidine-1-carboxylate (B). To a mixture of compound A (50 mg, 0.17 mmol) and benzyl ((5-(aminomethyl)thiophen-3-yl)(imino)methyl)carbamate (49 mg, 0.17 mmol) in DMF (1 mL) was added DIPEA (0.2 mL, 1.02 mmol) and T₃P (324 mg, 0.51 mmol, 50% wt. in EtOAc) at 0° C., and the mixture was stirred under N₂ atmosphere at 30° C. for 16 hours. The mixture was diluted with EtOAc and washed with saturated aq. NaHCO₃ solution. The aqueous layer was extracted with EtOAc twice. The combined organic layers were washed with brine, dried over anhydrous Na₂SO₄, filtered, and concentrated to dryness under reduced pressure. The residue was purified by flash chromatography on silica gel (PE:EtOAc=1:1) to give compound B (27 mg, yield 27.8%) as a light-yellow oil. LC/MS (ESI) m/z: 569 (M+H)⁺.

Step 2: benzyl ((5-(((2S,4R)-4-cyclohexylpyrrolidine-2-carboxamido)methyl)thiophen-3-yl)(imino)methyl)carbamate (C). A mixture of compound B (27 mg, 0.05 mmol) in HCl/1,4-dioxane (2 mL) was stirred at room temperature for 1.5 hours. The reaction mixture was concentrated to dryness under reduced pressure, co-evaporated with DCM twice, and dried under vacuum to give compound C (22 mg, yield 95.7%) as a white solid, which was used directly in the next step. LC/MS (ESI) m/z: 469 (M+H)⁺.

Step 3: benzyl ((5-(((2S,4R)-4-cyclohexyl-1-((4-phenoxybenzoyl)glycyl)pyrrolidine-2-carboxamido)methyl)thiophen-3-yl)(imino)methyl)carbamate (D). To a mixture of compound C (22 mg, 0.05 mmol) and (4-phenoxybenzoyl)glycine (13 mg, 0.05 mmol) in DMF (1 mL) was added DIPEA (0.05 mL, 0.3 mmol) and T₃P (95 mg, 0.15 mmol, 50% wt in EtOAc) at 0° C., and the mixture was stirred under N₂ atmosphere at 35° C. for 3.5 hours. The mixture was diluted with EtOAc and washed with saturated aq. NaHCO₃ solution. The aqueous layer was extracted with EtOAc twice. The combined organic layers were washed with brine, dried over anhydrous Na₂SO₄, filtered, and concentrated to dryness under reduced pressure. The residue was purified by flash chromatography on silica gel (DCM:MeOH=10:1) to give compound D (30 mg, yield 83.3%) as a yellow oil. LC/MS (ESI) m/z: 722 (M+H)⁺.

Step 4: (2S,4R)-N-((4-carbamimidoylthiophen-2-yl)methyl)-4-cyclohexyl-1-((4-phenoxy-benzoyl)glycyl)pyrrolidine-2-carboxamide (Compound 73). To a solution of compound D (30 mg, 0.04 mmol) in MeOH (2 mL) was added Pd/C (11 mg, 10% wt) and HCl/1,4-dioxane (cat., 2 drops) at room temperature under N₂ atmosphere, and the reaction mixture was stirred under a H₂ balloon at room temperature for 16 hours. The mixture was filtered, and the filtrate was concentrated to dryness under reduced pressure. The residue was purified by prep-HPLC to give Compound 73 (3.3 mg, yield 13.8%) as a white solid. ¹H NMR (400 MHz, CD₃OD) δ 8.52 (s, 1H), 8.20 (d, J=1.6 Hz, 1H), 7.84 (d, J=8.4 Hz, 2H), 7.44-7.38 (m, 3H), 7.20 (t, J=7.2 Hz, 1H), 7.06 (d, J=8.0 Hz, 2H), 7.00 (dd, J=9.6, 8.4 Hz, 2H), 4.58-4.57 (m, 3H), 4.35 (dd, J=10.0, 9.6 Hz, 1H), 4.27-4.13 (m, 4H), 4.02-3.92 (m, 1H), 2.55-2.37 (m, 1H), 2.13-1.99 (m, 1H), 1.76-1.66 (m, 5H), 1.57-1.52 (m, 1H), 1.30-1.19 (m, 4H), 1.11-0.99 (m, 2H); LC/MS (ESI) m/z: 588 (M+H)⁺.

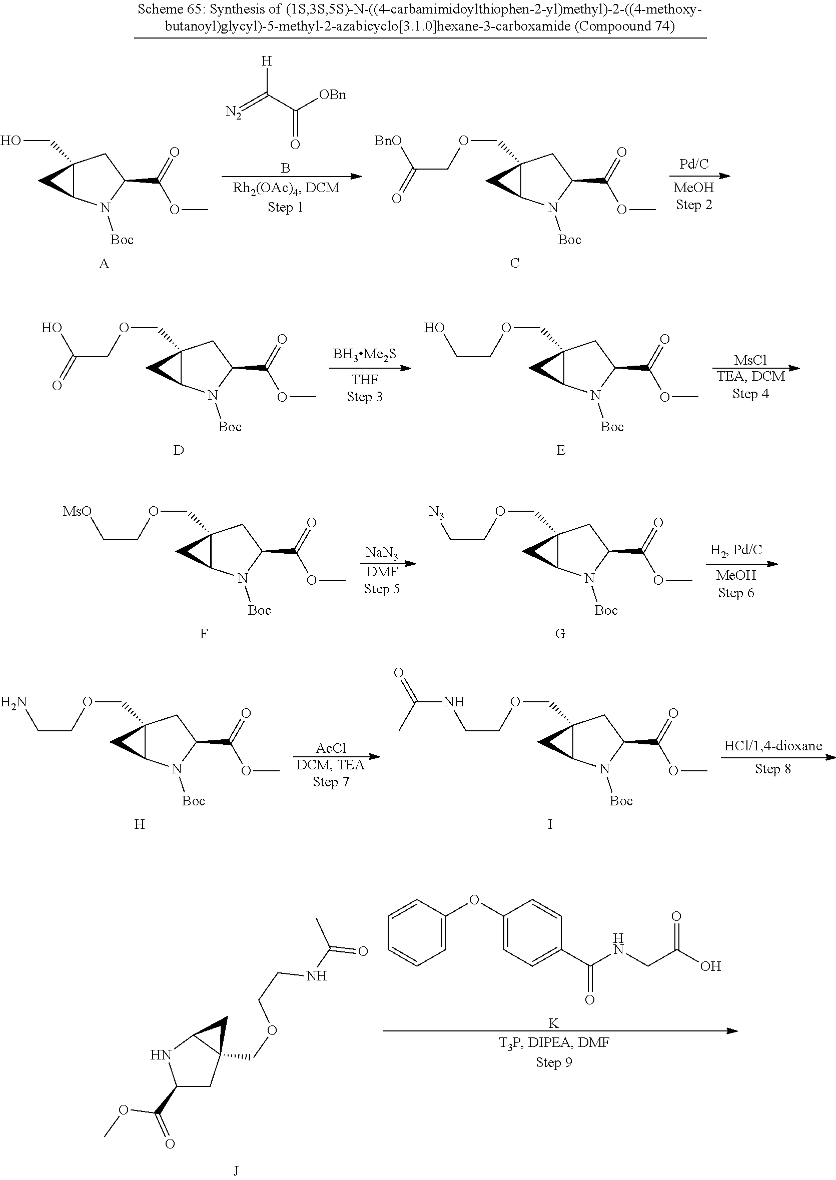

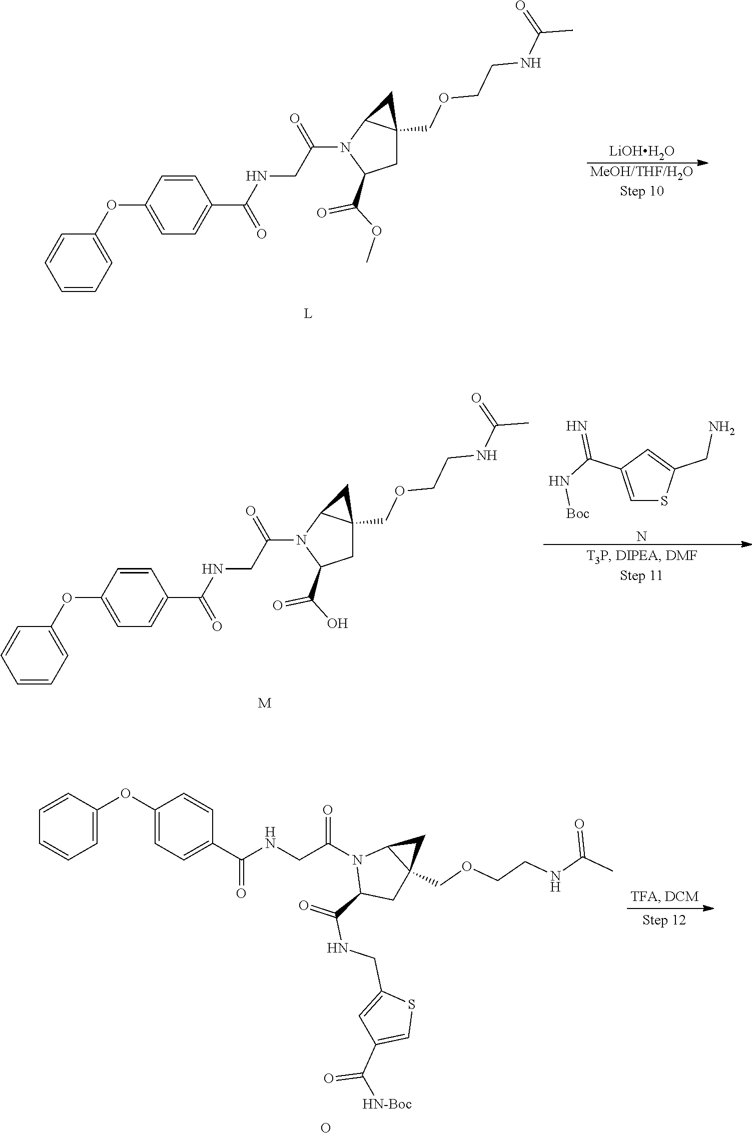

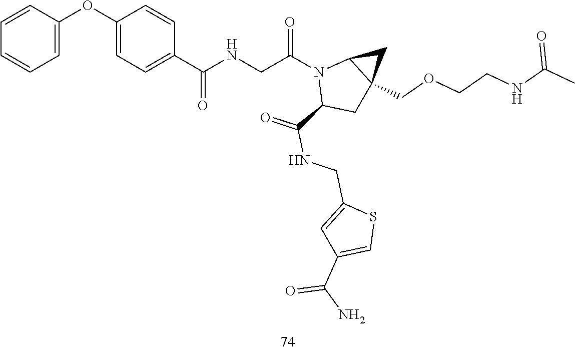 [SMILES: CC(=O)NCCOC[C@@]12C[C@@H]1N(C(=O)CNC(=O)c1ccc(Oc3ccccc3)cc1)[C@H](C(=O)NCc1cc(C(N)=O)cs1)C2 ; "74"]

Step 1: 2-(tert-butyl) 3-methyl (1S,3S,5R)-5-((2-(benzyloxy)-2-oxoethoxy)methyl)-2-azabicyclo[3.1.0]hexane-2,3-dicarboxylate (C). To a mixture of compound A (130 mg, 0.48 mmol) and Rh₂(OAc)₄ (21 mg, 0.05 mmol) in DCM (2 mL) was added a solution of benzyl 2-diazoacetate (B; 297 mg, 1.68 mmol) in DCM (1 mL) at 0° C., and the mixture was stirred under N₂ atmosphere at 25° C. for 16 hours. The mixture was diluted with water and extracted with EtOAc twice. The combined organic layers were dried over anhydrous Na₂SO₄, filtered, and concentrated to dryness under reduced pressure. The residue was purified by flash chromatography on silica gel (PE:EtOAc=10:1) to give compound C (172 mg, yield 85.6%) as a colorless oil. LC/MS (ESI) m/z: 420 (M+H)⁺.

Step 2: 2-(((1S,3S,5R)-2-(tert-butoxycarbonyl)-3-(methoxycarbonyl)-2 azabicyclo-[3.1.0]hexan-5-yl)methoxy)acetic acid (D). To a solution of compound C (172 mg, 0.41 mmol) in MeOH (5 mL) was added Pd/C (20 mg, 10% wt.) at room temperature under N₂ atmosphere and the reaction mixture was stirred under a H₂ balloon at room temperature for 3 hours. The mixture was filtered, and filtrate was concentrated to dryness under reduced pressure to give compound D (130 mg, yield 96.3%) as a colorless oil, which was used directly in next step. LC/MS (ESI) (m/z): 330 (M+H)⁺.

Step 3: 2-(tert-butyl) 3-methyl (1S,3S,5R)-5-((2-hydroxyethoxy)methyl)-2-azabicyclo-[3.1.0]hexane-2,3-dicarboxylate (E). To a solution of compound D (147 mg, 0.45 mmol) in THE (5 mL) was added BH₃·Me₂S (0.9 mL, 1.8 mmol, 2M in THF) at 0° C., and the mixture was stirred under N₂ atmosphere at 25° C. for 1 hour. The mixture was quenched with water and extracted with EtOAc twice. The combined organic layers were washed with brine, dried over anhydrous Na₂SO₄, filtered, and concentrated to dryness under reduced pressure. The residue was purified by flash chromatography on silica gel (PE:EtOAc=1:1) to give compound E (86 mg, yield 61.4%) as a colorless oil. LC/MS (ESI) m/z: 316 (M+H)⁺.

Step 4: 2-(tert-butyl) 3-methyl (1S,3S,5R)-5-((2-((methylsulfonyl)oxy)ethoxy)methyl)-2-azabicyclo[3.1.0]hexane-2,3-dicarboxylate (F). To a solution of compound E (140 mg, 0.44 mmol) in DCM (5 mL) was added TEA (0.5 mL, 3.6 mmol) and MsCl (0.14 mL, 1.76 mmol) at 0° C., and the mixture was stirred under N₂ atmosphere at 0° C. for 1 hour. The mixture was diluted with DCM and washed with brine. The organic layer was dried over anhydrous Na₂SO₄, filtered, and concentrated to dryness under reduced pressure to give compound F (170 mg, yield 97.1%) as a colorless oil, which was used directly in next step. LC/MS (ESI) m/z: 394 (M+H)⁺.

Step 5: 2-(tert-butyl) 3-methyl (1S,3S,5R)-5-((2-azidoethoxy)methyl)-2-azabicyclo-[3.1.0]hexane-2,3-dicarboxylate (G). To a solution of compound F (175 mg, 0.44 mmol) in DMF (3 mL) was added NaN₃ (72 mg, 1.1 mmol) and the mixture was stirred at 40° C. for 16 hours. The mixture was diluted with water and extracted with DCM twice. The combined organic layers were washed with brine, dried over anhydrous Na₂SO₄, filtered, and concentrated to dryness under reduced pressure. The residue was purified by chromatography on silica gel (PE:EtOAc=5:1) to give compound G (134 mg, yield 88.7%) as a colorless oil. LC/MS (ESI) m/z: 341(M+H)⁺.

Step 6: 2-(tert-butyl) 3-methyl (1S,3S,5R)-5-((2-aminoethoxy)methyl)-2-azabicyclo-[3.1.0]hexane-2,3-dicarboxylate (H). To a solution of compound G (134 mg, 0.39 mmol) in MeOH (5 mL) was added Pd/C (20 mg, 10% wt) under N₂ atmosphere at room temperature, and the reaction mixture was stirred under a H₂ balloon at room temperature for 3 hours. The mixture was filtered, and the filtrate was concentrated to dryness under reduced pressure to give compound H (110 mg, yield 89.4%) as a colorless oil, which was used directly in next step. LC/MS (ESI) (m/z): 315 (M+H)⁺.

Step 7: 2-(tert-butyl) 3-methyl (1S,3S,5R)-5-((2-acetamidoethoxy)methyl)-2-azabicyclo-[3.1.0]hexane-2,3-dicarboxylate (1). To a solution of compound H (110 mg, 0.35 mmol) in DCM (5 mL) was added TEA (0.3 mL, 2.1 mmol) and AcCl (0.07 mL, 1.05 mmol) at 0° C., and the mixture was stirred under N₂ atmosphere at 0° C. for 1 hour. The mixture was diluted with DCM and washed with brine, dried over anhydrous Na₂SO₄, filtered, and concentrated to dryness under reduced pressure. The residue was purified by flash chromatography on silica gel (DCM:MeOH=95:5) to give compound I (86 mg, yield 69.3%) as a light-yellow oil. LC/MS (ESI) m/z: 357 (M+H)⁺.

Step 8: methyl (1S,3S,5R)-5-((2-acetamidoethoxy)methyl)-2-azabicyclo[3.1.0]hexane-3-carboxylate (J). A mixture of compound I (86 mg, 0.24 mmol) in HCl/1,4-dioxane (5 mL) was stirred at room temperature for 1 hour. The reaction mixture was concentrated to dryness under reduced pressure, co-evaporated with DCM twice, and dried under vacuum to give compound J (60 mg, yield 96.8%) as a yellow oil, which was used directly in the next step. LC/MS (ESI) m/z: 257 (M+H)⁺.

Step 9: methyl (1S,3S,5R)-5-((2-acetamidoethoxy)methyl)-2-((4-phenoxybenzoyl)glycyl)-2-azabicyclo[3.1.0]hexane-3-carboxylate (L). To a mixture of compound J (62 mg, 0.24 mmol) and (4-phenoxybenzoyl)glycine (K; 72 mg, 0.26 mmol) in DMF (5 mL) was added DIPEA (0.24 mL, 1.44 mmol) and T₃P (460 mg, 0.72 mmol, 50% wt. in EtOAc) at 0° C., and the mixture was stirred under N₂ atmosphere at 25° C. for 16 hours. The mixture was diluted with H₂O and extracted with EtOAc twice. The combined organic layers were washed with saturated aq. NaHCO₃ solution and brine, dried over anhydrous Na₂SO₄, filtered, and concentrated to dryness under reduced pressure. The residue was purified by flash chromatography on silica gel (DCM:MeOH=20:1) to give compound L (92 mg, yield 74.8%) as a yellow oil. LC/MS (ESI) m/z: 510 (M+H)⁺.

Step 10: (1S,3S,5R)-5-((2-acetamidoethoxy)methyl)-2-((4-phenoxybenzoyl)glycyl)-2-azabicyclo[3.1.0]hexane-3-carboxylic acid (M). To a solution of compound M (92 mg, 0.18 mmol) in MeOH (2 mL), THE (1 mL) and H₂O (1 mL) was added LiOH·H₂O (11 mg, 0.27 mmol) at 0° C., and the mixture was stirred at room temperature for 2 hours. The aqueous layer was acidified with 1 N aq. HCl solution to pH˜3 and extracted with CHCl₃/i-PrOH (v/v=3/1) five times. The combined organic layers were washed with brine, dried over anhydrous Na₂SO₄, and concentrated to dryness under reduced pressure to give compound M (85 mg, yield 95.5%) as a light-yellow oil, which was used directly in the next step. LC/MS (ESI) (m/z): 496 (M+H)⁺.

Step 11: tert-butyl ((5-(((1S,3S,5R)-5-((2-acetamidoethoxy)methyl)-2-((4-phenoxybenzoyl)-glycyl)-2-azabicyclo[3.1.0]hexane-3-carboxamido)methyl)thiophen-3-yl)(imino)methyl)carbamate (0). To a mixture of compound M (80 mg, 0.10 mmol) and tert-butyl ((5-(aminomethyl)thiophen-3-yl)(imino)methyl)carbamate (N; 62 mg, 0.15 mmol) in DMF (5 mL) was added DIPEA (0.16 mL, 0.60 mmol) and T₃P (308 mg, 0.30 mmol, 50% wt. in EtOAc) at 0° C., and the mixture was stirred under N₂ atmosphere at 25° C. for 3 hours. The mixture was diluted with H₂O and extracted with EtOAc twice. The combined organic layers were washed with saturated aq. NaHCO₃ solution and brine, dried over anhydrous Na₂SO₄, filtered, and concentrated to dryness under reduced pressure. The residue was purified by flash chromatography on silica gel (DCM:MeOH=9:1) to give compound O (42 mg, yield 35.6%) as a yellow oil. LC/MS (ESI) m/z: 733 (M+H)⁺.

Step 10: (1S,3S,5R)-5-((2-acetamidoethoxy)methyl)-N-((4-carbamimidoylthiophen-2-yl)methyl)-2-((4-phenoxybenzoyl)glycyl)-2-azabicyclo[3.1.0]hexane-3-carboxamide (Compound 74). To a solution of compound O (42 mg, 0.06 mmol) in DCM (4 mL) was added TFA (1 mL) at 0° C., and the solution was stirred at room temperature for 1 hour. The reaction mixture was concentrated to dryness under reduced pressure, co-evaporated with DCM twice, dried under vacuum, and further purified by prep-HPLC to give Compound 74 (5 mg, yield 13.9%) as a white solid. ¹H NMR (400 MHz, CD₃OD) δ 8.21 (d, J=1.6 Hz, 1H), 7.84 (d, J=8.8 Hz, 2H), 7.41 (dd, J=9.3, 6.7 Hz, 3H), 7.21 (t, J=7.5 Hz, 1H), 7.08-7.04 (m, 2H), 7.02-6.98 (m, 2H), 4.84-4.83 (m, 1H), 4.55 (d, J=4.9 Hz, 2H), 4.39-4.29 (m, 2H), 3.62-3.57 (m, 2H), 3.53 (t, J=5.6 Hz, 2H), 3.46 (d, J=10.6 Hz, 2H), 3.36 (d, J=5.5 Hz, 2H), 2.67 (t, J=12.8 Hz, 1H), 2.12 (dd, J=13.4, 3.5 Hz, 1H), 1.94 (s, 3H), 1.22 (dd, J=5.9, 2.6 Hz, 1H), 1.01 (t, J=5.5 Hz, 1H); LC/MS (ESI) m/z: 633 (M+H)⁺.

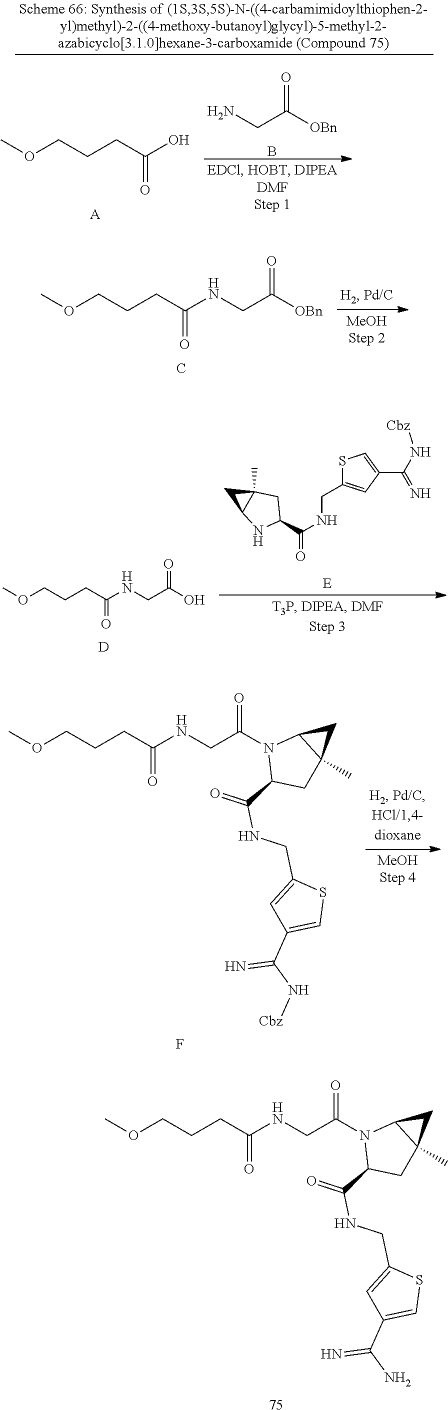

Step 1: benzyl (4-methoxybutanoyl)glycinate (C). To a mixture of compound A (100 mg, 0.85 mmol) and benzyl glycinate (B; 256 mg, 1.28 mmol) in DMF (5 mL) was added DIPEA (0.84 mL, 5.1 mmol), EDCI (325 mg, 1.7 mmol), and HOBT (171 mg, 1.28 mmol) at 0° C., and the mixture was stirred under N₂ atmosphere at 25° C. for 16 hours. The mixture was diluted with H₂O and extracted with EtOAc twice. The combined organic layers were dried over anhydrous Na₂SO₄, filtered, and concentrated to dryness under reduced pressure. The residue was purified by flash chromatography on silica gel (PE:EtOAc=1:1) to give compound C (106 mg, yield 47.3%) as a yellow solid. LC/MS (ESI) m/z: 266 (M+H)⁺.

Step 2: (4-methoxybutanoyl)glycine (D). To a solution of compound C (106 mg, 0.4 mmol) in MeOH (5 mL) was added Pd/C (20 mg, 10% wt) at room temperature under N₂ atmosphere and the reaction mixture was stirred under a H₂ balloon at room temperature for 3 hours. The mixture was filtered, and the filtrate was concentrated to dryness under reduced pressure to give compound D (65 mg, yield 92.8%) as colorless oil, which was used directly in next step. LC/MS (ESI) (m/z): 176 (M+H)⁺.

Step 3: benzyl (imino(5-(((1S,3S,5S)-2-((4-methoxybutanoyl)glycyl)-5-methyl-2-azabicyclo-[3.1.0]hexane-3-carboxamido)methyl)thiophen-3-yl)methyl)carbamate (F) To a mixture of compound D (35 mg, 0.2 mmol) and benzyl (imino(5-(((1S,3S,5S)-5-methyl-2-azabicyclo[3.1.0]hexane-3-carboxamido)methyl)thiophen-3-yl)methyl)carbamate (E; 82.4 mg, 0.2 mmol) in DMF (5 mL) was added DIPEA (0.2 mL, 1.2 mmol) and T₃P (382 mg, 0.6 mmol, 50% wt in EtOAc) at 0° C., and the mixture was stirred under N₂ atmosphere at 25° C. for 5 hours. The mixture was diluted with H₂O and extracted with CHCl₃/i-PrOH (v/v=3/1) twice. The combined organic layers were dried over anhydrous Na₂SO₄, filtered, and concentrated to dryness under reduced pressure. The residue was purified by flash chromatography on silica gel (DCM:MeOH=9:1) to give compound F (80 mg, yield 70.2%) as a white semi-solid. LC/MS (ESI) m/z: 570 (M+H)⁺.

Step 4: (1S,3S,5S)-N-((4-carbamimidoylthiophen-2-yl)methyl)-2-((4-methoxybutanoyl)-glycyl)-5-methyl-2-azabicyclo[3.1.0]hexane-3-carboxamide (Compound 75) To a solution of compound F (40 mg, 0.07 mmol) in MeOH (5 mL) was added Pd/C (20 mg, 10% wt.) and HCl/1,4-dioxane (cat., 2 drops) at room temperature under N₂ atmosphere and the reaction mixture was stirred under a H₂ balloon at room temperature for 3 hours. The mixture was filtered and the filtrate was concentrated to dryness under reduced pressure. The residue was purified by prep-HPLC to give Compound 75 (5 mg, yield 16.3%) as a white solid. ¹H NMR (400 MHz, CD₃OD) δ 8.51 (s, 1H), 8.24-8.23 (d, J=4.0 Hz, 1H), 7.41 (s, 1H), 4.79 (dd, J=11.4, 3.3 Hz, 1H), 4.59-4.46 (m, 2H), 4.22-4.08 (m, 2H), 3.40 (t, J=6.3 Hz, 2H), 3.33 (d, J=2.4 Hz, 1H), 3.31 (s, 3H), 2.45-2.30 (m, 3H), 2.15 (dd, J=13.4, 3.3 Hz, 1H), 1.88-1.80 (m, 2H), 1.28 (s, 3H), 1.13 (dd, J=5.7, 2.4 Hz, 1H), 0.80 (t, J=5.4 Hz, 1H); LC/MS (ESI) m/z: 436 (M+H)⁺.

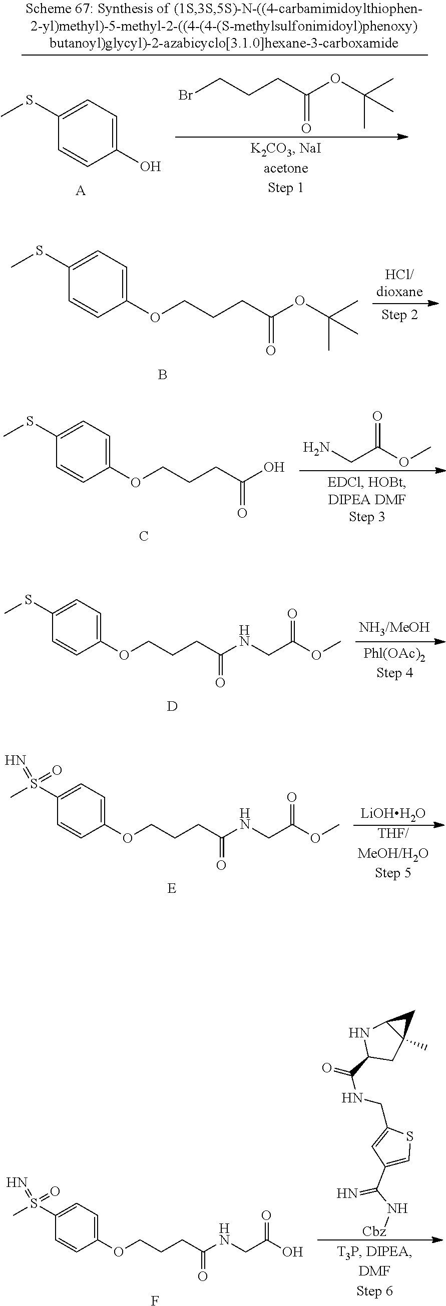

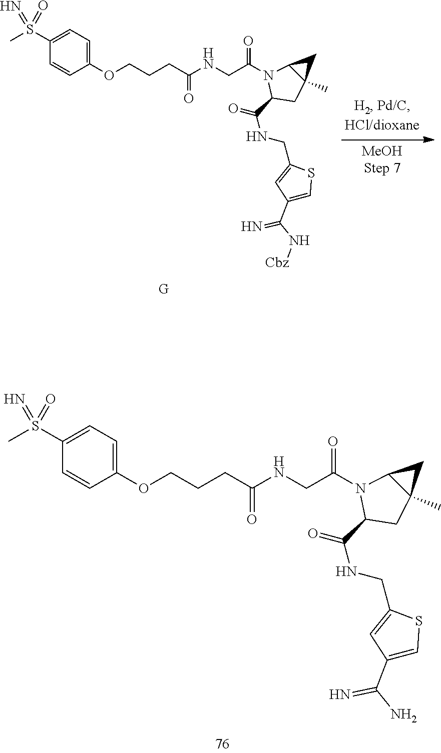

Step 1: tert-butyl 4-[4-(methylsulfanyl)phenoxy]butanoate (B). To a solution of compound A (1 g, 7.13 mmol) in acetone (15 mL) was added tert-butyl 4-bromobutanoate (1.59 g, 7.13 mmol), K₂CO₃ (1.18 g, 8.56 mmol), and NaI (107 mg, 0.713 mmol) at room temperature, and the reaction was stirred at 60° C. for 16 hours. The resulting mixture was diluted with water and extracted with EtOAc twice. The combined organic layers were washed with brine, dried over anhydrous Na₂SO₄, filtered, and concentrated to dryness under reduced pressure. The residue was purified by flash chromatography on silica gel (PE:EtOAc=10:1 to 5:1) to give compound B (1.43 g, yield 71.0%) as a white solid. LC/MS (ESI) m/z: 283 (M+H)⁺.

Step 2: 4-[4-(methylsulfanyl)phenoxy]butanoic acid (C). A mixture of compound B (1.43 g, 5.06 mmol) in HCl/1,4-dioxane (7 mL) was stirred at room temperature for 2 hours, and the resulting mixture was concentrated to dryness under reduced pressure to give compound C (1.04 g, yield 90.7%) as a white solid. LC/MS (ESI) m/z: 227 (M+H)⁺.

Step 3: methyl 2-{4-[4-(methylsulfanyl)phenoxy]butanamido}acetate (4). To a mixture of compound C (1.04 g, 4.60 mmol) and methyl 2-aminoacetate (614 mg, 6.89 mmol) in DMF (10 mL) was added DIPEA (2.97 g, 23.0 mmol), EDCI (1.76 g, 9.19 mmol), and HOBt (930 mg, 6.89 mmol) at room temperature, and the resulting mixture was stirred under N₂ atmosphere at room temperature for 3 hours. The mixture was diluted with water extracted with EtOAc twice. The combined organic layers were washed with brine, dried over anhydrous Na₂SO₄, filtered, and concentrated to dryness under reduced pressure. The residue was purified by flash chromatography on silica gel (PE:EtOAc=10:1) to give compound D (1.01 g, yield 73.9%) as a white solid. LC/MS (ESI) m/z: 298 (M+H)⁺.

Step 4: methyl (4-(4-(S-methylsulfonimidoyl)phenoxy)butanoyl)glycinate (E). To a solution of compound D (1.01 g, 3.40 mmol) in MeOH (10 mL) was added NH₃/MeOH (0.73 mL, 5.10 mmol) and Phl(OAc)₂ (2.52 g, 7.81 mmol) at room temperature, and the reaction was stirred at room temperature for 2 hours. The mixture was diluted with water and extracted with EtOAc twice. The combined organic layers were washed with brine, dried over anhydrous Na₂SO₄, filtered, and concentrated to dryness under reduced pressure. The residue was purified by flash chromatography on silica gel (DCM:MeOH=10:1) to give compound E (975 mg, yield 87.4%) as a white solid. LC/MS (ESI) m/z: 329 (M+H)⁺.

Step 5: (4-(4-(S-methylsulfonimidoyl)phenoxy)butanoyl)glycine (F). To a solution of compound E (975 mg, 2.97 mmol) in THE (8 mL), MeOH (2 mL), and H₂O (2 mL) was added LiOH·H₂O (374 mg, 8.91 mmol) at 0° C., and the reaction was stirred at room temperature for 16 hours. The resulting mixture was acidified with 2 N aq. HCl solution to pH˜3 and extracted with EtOAc twice. The combined organic layers were washed with brine, dried over anhydrous Na₂SO₄, filtered, and concentrated to dryness under reduced pressure to give compound F (900 mg, yield 96.4%) as a white solid, which was used directly in the next step. LC/MS (ESI) m/z: 315 (M+H)⁺.

Step 6: benzyl (imino(5-(((1S,3S,5S)-5-methyl-2-((4-(4-(S-methylsulfonimidoyl)-phenoxy)butanoyl)glycyl)-2-azabicyclo[3.1.0]hexane-3-carboxamido)methyl)thiophen-3-yl)methyl)-carbamate (G). To a mixture of compound F (38.1 mg, 0.121 mmol) and benzyl (imino(5-(((1 S,3S,5S)-5-methyl-2-azabicyclo[3.1.0]hexane-3-carboxamido)-methyl)thiophen-3-yl)methyl)carbamate (100 mg, 0.121 mmol) in DMF (5 mL) was added DIPEA (94 mg, 0.727 mmol) and T₃P (231 mg, 0.364 mmol, 50% wt in EtOAc) at room temperature, and the reaction was stirred under N₂ atmosphere at room temperature for 3 hours. The mixture was diluted with water and extracted with EtOAc twice. The combined organic layers were washed with saturated aq. NaHCO₃ solution and brine, dried over anhydrous Na₂SO₄, filtered, and concentrated to dryness under reduced pressure. The residue was purified by flash chromatography on silica gel (DCM:MeOH=10:1) to give compound G (85 mg, yield 98.9%) as a white solid. LC/MS (ESI) m/z: 709 (M+H)⁺.

Step 7: (1S,3S,5S)-N-((4-carbamimidoylthiophen-2-yl)methyl)-5-methyl-2-((4-(4-(S-methyl-sulfonimidoyl)phenoxy)butanoyl)glycyl)-2-azabicyclo[3.1.0]hexane-3-carboxamide (Compound 76). To a solution of compound 7 (85 mg, 0.12 mmol) in MeOH (3 mL) was added Pd/C (20 mg, 10% wt) and HCl/1,4-dioxane (cat., 2 drops) at room temperature under N₂ atmosphere and the reaction was stirred under a H₂ balloon at room temperature for 16 hours. The mixture was filtered and the filtrate was concentrated to dryness under reduced pressure. The residue was purified by prep-HPLC to give Compound 76 (16 mg, yield 23.2%) as white solid. ¹H NMR (400 MHz, CD₃OD) δ 8.25 (dd, J=6.9, 1.5 Hz, 1H), 8.08-8.04 (m, 2H), 7.43 (d, J=11.3 Hz, 1H), 7.29 (dd, J=9.6, 2.6 Hz, 2H), 4.81-4.77 (m, 1H), 4.58-4.50 (m, 2H), 4.28-4.09 (m, 4H), 3.72 (s, 3H), 3.34 (d, J=2.4 Hz, 1H), 2.49 (t, J=7.3 Hz, 2H), 2.40 (t, J=12.4 Hz, 1H), 2.18-2.11 (m, 3H), 1.28 (s, 3H), 1.17-1.15 (m, 1H), 0.80 (t, J=5.4 Hz, 1H); LC/MS (ESI) m/z: 575 (M+H)⁺.

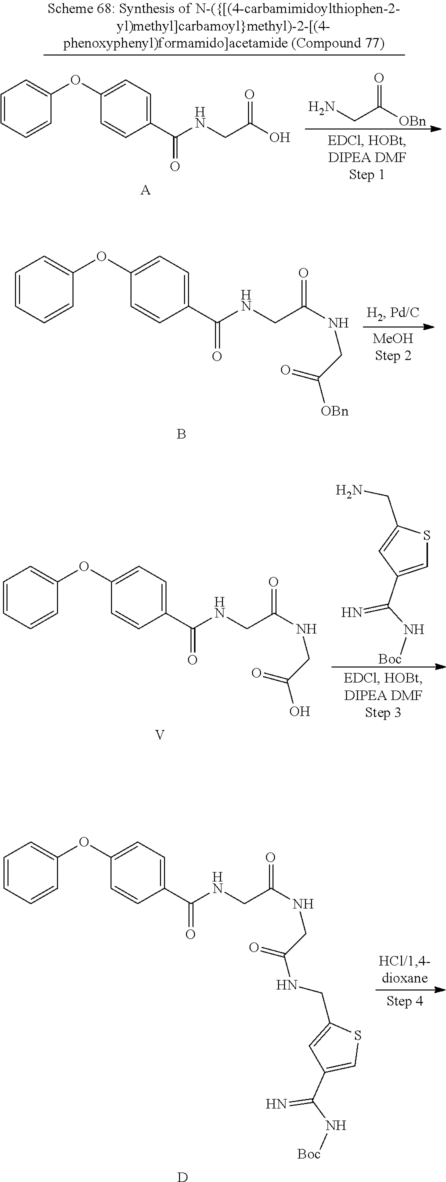

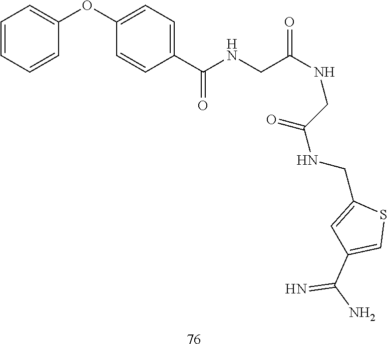 [SMILES: N=C(N)c1csc(CNC(=O)CNC(=O)CNC(=O)c2ccc(Oc3ccccc3)cc2)c1 ; "76"]

Step 1: benzyl (4-phenoxybenzoyl)glycylglycinate (B). To a mixture of compound A (300 mg, 1.11 mmol) and benzyl glycinate hydrochloride (274 mg, 1.66 mmol) in DMF (10 mL) was added DIPEA (714 mg, 5.53 mmol), EDCI (424 mg, 2.21 mmol), and HOBt (224 mg, 1.66 mmol) at room temperature, and the reaction was stirred under N₂ atmosphere at room temperature for 3 hours. The mixture was diluted with water and extracted with EtOAc twice. The combined organic layers were washed with brine, dried over anhydrous Na₂SO₄, filtered and concentrated to dryness under reduced pressure. The residue was purified by flash chromatography on silica gel (PE:EtOAc=10:1 to 1:1) to give compound B (440 mg, yield 95.1%) as a white solid. LC/MS (ESI) m/z: 419 (M+H)⁺.

Step 2: (4-phenoxybenzoyl)glycylglycine (V). To a solution of compound B (440 mg, 1.05 mmol) in MeOH (10 mL) was added Pd/C (40 mg, 10% wt.) at room temperature under N₂ atmosphere, and the reaction was stirred under a H₂ balloon at room temperature for 3 hours. The mixture was filtered, and the filtrate was concentrated to dryness under reduced pressure to give compound C (130 mg, yield 37.6%) as a white solid, which was used directly in the next step. LC/MS (ESI) m/z: 329 (M+H)⁺.

Step 3: tert-butyl N-{5-[(2-{2-[(4-phenoxyphenyl)formamido]acetamido}-acetamido)-methyl]thiophene-3-carboximidoyl}carbamate (D). To a mixture of compound 3 (130 mg, 0.396 mmol) and tert-butyl ((5-(aminomethyl)thiophen-3-yl)(imino)methyl)carbamate (152 mg, 0.594 mmol) in DMF (10 mL) was added DIPEA (256 mg, 1.98 mmol), EDCI (152 mg, 0.792 mmol), and HOBt (80.2 mg, 0.594 mmol) at room temperature and the reaction was stirred under N₂ atmosphere at room temperature for 3 hours. The mixture was diluted with water and extracted with EtOAc twice. The combined organic layers were washed with brine, dried over anhydrous Na₂SO₄, filtered, and concentrated to dryness under reduced pressure. The residue was purified by flash chromatography on silica gel (DCM:MeOH=10:1) to give compound D (130 mg, yield 58.0%) as a white solid. LC/MS (ESI) m/z: 566 (M+H)⁺.

Step 4: N-(2-((2-(((4-carbamimidoylthiophen-2-yl)methyl)amino)-2-oxoethyl)amino)-2-oxoethyl)-4-phenoxybenzamide (Compound 77). To a solution of compound D (65 mg, 0.115 mmol) in 1,4-dioxane (2 mL) was added HCl/1,4-dioxane (1 mL) at 0° C. and the reaction was stirred at room temperature for 16 hours. The resulting mixture was concentrated to dryness under reduced pressure, co-evaporated with DCM twice and dried under vacuum. The residue was purified by prep-HPLC to give Compound 77 (12 mg, yield 22.6%) as a white solid. ¹H NMR (400 MHz, CD₃OD) δ 8.22 (d, J=1.6 Hz, 1H), 7.86-7.83 (m, 2H), 7.44-7.40 (m, 3H), 7.21 (t, J=7.4 Hz, 1H), 7.07-7.04 (m, 2H), 7.01-6.99 (m, 2H), 4.61 (s, 2H), 4.03 (s, 2H), 3.92 (s, 2H); LC/MS (ESI) m/z: 466 (M+H)⁺.

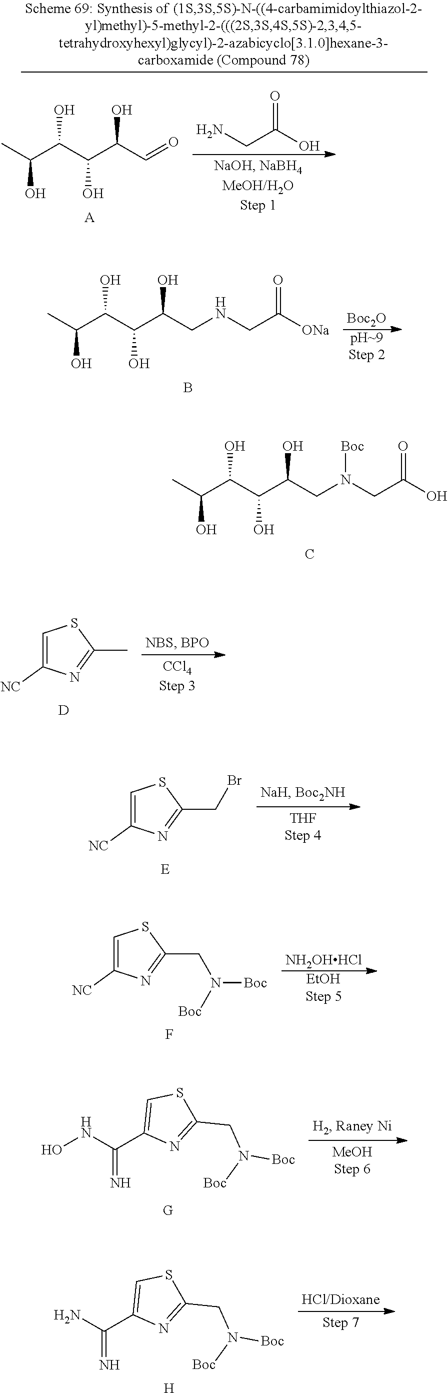

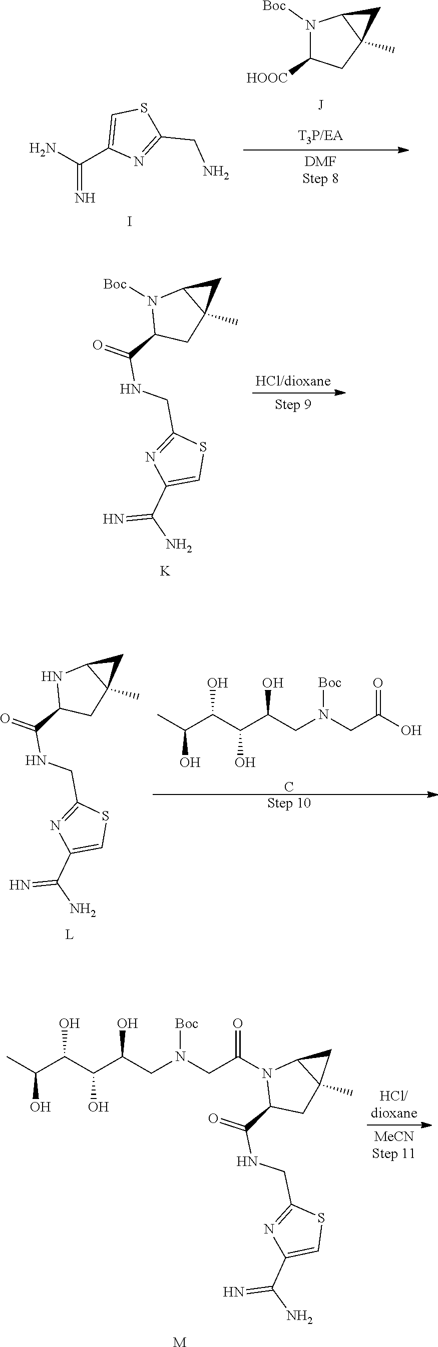

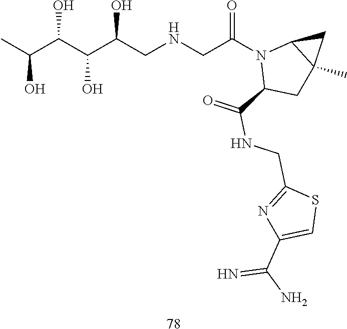 [SMILES: C[C@H](O)[C@H](O)[C@@H](O)[C@@H](O)CNCC(=O)N1[C@H]2C[C@@]2(C)C[C@H]1C(=O)NCc1nc(C(=N)N)cs1 ; "78"]

Step 1: sodium ((2S,3S,4S,5S)-2,3,4,5-tetrahydroxyhexyl)glycinate (B). To a solution of sodium hydroxide (1.22 g, 30.46 mmol) in methanol/H₂O (10 mL, V:V=1:1) was added glycine (2.29 g, 30.46 mmol). The mixture was stirred at room temperature until glycine was completely dissolved. Then (2R,3R,4S,5S)-2,3,4,5-tetrahydroxyhexanal (A; 5.0 g, 30.46 mmol) was added to the above mixture. The mixture was stirred at room temperature until compound A completely dissolved and further stirred at 50 to 60° C. for 5 hours under N₂ atmosphere. NaBH₄ (3.46 g, 91.38 mmol) was added in portions to the above mixture at room temperature and the resulting mixture was stirred at room temperature for 24 hours. The reaction mixture was then cooled down to 0° C. and the mixture was adjusted to pH˜9 and used directly in next step without further purification.

Step 2: N-(tert-butoxycarbonyl)-N-((2S,3S,4S,5S)-2,3,4,5-tetrahydroxyhexyl)glycine (C) To the above mixture was added di(tert-butyl) carbonate (19.94 g, 91.38 mmol) at 0° C. and the mixture was stirred at room temperature overnight. The reaction mixture was adjusted to pH=4 to 5 with concentrated aq. HCl. The precipitates were removed by filtration, and the filtrate was concentrated to dryness. The residue was diluted with ethanol, and the precipitates formed were removed by filtration. The procedure was repeated five times and the solution was concentrated to dryness to give N-(tert-butoxycarbonyl)-N-((2S,3S,4S,5S)-2,3,4,5-tetrahydroxyhexyl)glycine (C; 1.76 g, 17.9%) as a white solid. LC/MS (ESI) m/z: 322 (M−H)⁻.

Step 3: 2-(bromomethyl)thiazole-4-carbonitrile (E). To a solution of 2-methylthiazole-4-carbonitrile (D; 1.0 g, 8.05 mmol) in carbon tetrachloride (5 mL) was added NBS (1.4 g, 8.05 mmol) and BPO (97.5 mg, 0.403 mmol) at room temperature. The mixture was stirred at 90° C. under N₂ atmosphere overnight. The mixture was diluted with water and extracted with dichloromethane (2×10 mL). The combined organic layers were washed with brine, dried over anhydrous Na₂SO₄, filtered, and concentrated to dryness under reduced pressure. The residue was purified by column chromatography on silica gel (EtOAc/PE=0:1 to 1:25) to give 2-(bromomethyl)thiazole-4-carbonitrile (E; 600 mg, 36.7% yield) as a colorless oil. LC/MS (ESI) m/z: 204 (M+H)⁺.

Step 4: tert-butyl N-(tert-butoxycarbonyl)-N-[(4-cyano-1,3-thiazol-2-yl)methyl]carbamate (F). To a solution of di-tert-butyl iminodicarboxylate (833.5 mg, 3.84 mmol) in anhydrous THE (10 mL) was added NaH (160 mg, 60% wt) at 0° C., under N₂ atmosphere and the mixture was stirred at 0° C. for 20 minutes. 2-(bromomethyl)thiazole-4-carbonitrile (E; 600 mg, 2.95 mmol) in THE (3 mL) was added to the above mixture, and the resulting mixture was stirred at 25° C. for another 16 hours under N₂ atmosphere. The mixture was quenched with saturated aq. NH₄Cl solution and extracted with ethyl acetate (3×20 mL). The combined organic layers were washed with brine, dried over anhydrous Na₂SO₄, filtered, and concentrated to dryness under reduced pressure. The residue was purified by flash column chromatography via silica gel (EtOAc=0:1 to 1 25) to give tert-butyl N-(tert-butoxycarbonyl)-N-[(4-cyano-1,3-thiazol-2-yl)methyl]carbamate (F; 720 mg, 71.8% yield) as a white solid. LC/MS (ESI) m/z: 362 (M+23)*.

Step 5: tert-butyl N-(tert-butoxycarbonyl)-N-{[4-(N-hydroxycarbamimidoyl)-1,3-thiazol-2-yl]methyl}carbamate (G). To a mixture of tert-butyl N-(tert-butoxycarbonyl)-N-[(4-cyano-1,3-thiazol-2-yl)methyl]carbamate (F; 720 mg, 2.12) in methanol (8 mL) was added DIPEA (820.9 mg, 6.36 mmol) and NH₂OH·HCl (442.2 mg, 6.36 mmol) at 0° C., and the mixture was stirred at 25° C. for 16 hours. After completion of the reaction, the mixture was concentrated to dryness and extracted with dichloromethane (2×10 mL). The combined organic layers were washed with water and brine, dried over anhydrous Na₂SO₄, and concentrated to dryness under reduced pressure. The residue was purified by flash column chromatography on silica gel (EtOAc:PE=0:1 to 1:1) to give tert-butyl N-(tert-butoxycarbonyl)-N-{[4-(N-hydroxycarbamimidoyl)-1,3-thiazol-2-yl]methyl}carbamate (G; 780 mg, 98.7% yield) as a white solid. LC/MS (ESI) m/z: 373 (M+H)⁺.

Step 6: tert-butyl N-(tert-butoxycarbonyl)-N-[(4-carbamimidoyl-1,3-thiazol-2-yl)methyl]carbamate (H). To a solution of tert-butyl N-(tert-butoxycarbonyl)-N-{[4-(N-hydroxycarbamimidoyl)-1,3-thiazol-2-yl]methyl}carbamate (G; 780 mg, 2.09 mmol) in methanol (8 mL) was added Raney Ni in water (1 mL) and acetic acid (0.5 mL), and the mixture was stirred under a H₂ balloon at 30° C. for 16 hours. The reaction mixture was filtered, and the filtrate was concentrated to dryness. The residue was purified by flash column chromatography on silica gel (MeOH:DCM=0:1 to 1:10) to give tert-butyl N-(tert-butoxycarbonyl)-N-[(4-carbamimidoyl-1,3-thiazol-2-yl)methyl]carbamate (H; 420 mg, 56.2% yield) as a yellow oil. LC/MS (ESI) m/z: 357 (M+H)⁺.

Step 7: tert-butyl N-(tert-butoxycarbonyl)-N-[(4-carbamimidoyl-1,3-thiazol-2-yl)methyl]carbamate (1). A mixture of tert-butyl N-(tert-butoxycarbonyl)-N-[(4-carbamimidoyl-1,3-thiazol-2-yl)methyl]carbamate (105 mg, 0.295 mmol) and HCl/1,4-dioxane (2 mL) was stirred at room temperature for 2 hours. The reaction mixture was concentrated to dryness under reduced pressure, co-evaporated with dichloromethane, and dried under vacuum to give tert-butyl N-(tertbutoxycarbonyl)-N-[(4-carbamimidoyl-1,3-thiazol-2-yl)methyl]carbamate (I; 46 mg, quantitative yield) as yellow solid, which was used directly in the next step without further purification. LC/MS (ESI) m/z: 157 (M+H)⁺.

Step 8: tert-butyl (1S,3S,5S)-3-(((4-carbamimidoylthiazol-2-yl)methyl)carbamoyl)-5-methyl-2-azabicyclo[3.1.0]hexane-2-carboxylate (K). To a mixture of (1 S,3S,5S)-2-(tert-butoxycarbonyl)-5-methyl-2-azabicyclo[3.1.0]hexane-3-carboxylic acid (J; 60 mg, 0.249 mmol) and tert-butyl N-(tert-butoxycarbonyl)-N-[(4-carbamimidoyl-1,3-thiazol-2-yl)methyl]carbamate (I; 42.7 mg, 0.274 mmol) in DMF (3 mL) was added T₃P (316.3 g, 0.497 mmol, 50% in ethyl acetate) and DIPEA (128 mg, 0.995 mmol). The mixture was stirred under N₂ atmosphere at room temperature overnight. The reaction was quenched with saturated aq. NH₄Cl solution. The resulting mixture was extracted with ethyl acetate (3×20 mL). The combined organic layers were washed with water (2×20 mL) and brine (2×20 mL), dried over anhydrous Na₂SO₄, filtered, and concentrated to dryness under reduced pressure. The residue was purified by flash column chromatography on silica gel (MeOH:DCM=1:10 to 1:6) to give tert-butyl (1 S,3S,5S)-3-(((4-carbamimidoylthiazol-2-yl)methyl)carbamoyl)-5-methyl-2-azabicyclo[3.1.0]hexane-2-carboxylate (K; 31 mg, 32.8% yield) as yellow oil. LC/MS (ESI) m/z: 380 (M+H)⁺.

Step 9: (1S,3S,5S)-N-((4-carbamimidoylthiazol-2-yl)methyl)-5-methyl-2-azabicyclo[3.1.0]hexane-3-carboxamide (L). A mixture of tert-butyl (1 S,3S,5S)-3-(((4-carbamimidoylthiazol-2-yl)methyl)carbamoyl)-5-methyl-2-azabicyclo[3.1.0]hexane-2-carboxylate (K; 31 mg, 0.082 mmol) and HCl/1,4-dioxane (1 mL) was stirred at room temperature for 1 hour. The reaction mixture was concentrated to dryness under reduced pressure, co-evaporated with dichloromethane, and dried under vacuum to give (1 S,3S,5S)-N-((4-carbamimidoylthiazol-2-yl)methyl)-5-methyl-2-azabicyclo[3.1.0]hexane-3-carboxamide (L; 23 mg, quantitative yield) as a white solid, which was used directly in the next step without further purification. LC/MS (ESI) m/z: 280 (M+H)⁺.

Step 10: tert-butyl (2-((1S,3S,5S)-3-(((4-carbamimidoylthiazol-2-yl)methyl)carbamoyl)-5-methyl-2-azabicyclo[3.1.0]hexan-2-yl)-2-oxoethyl)((2S,3S,4S,5S)-2,3,4,5-tetrahydroxyhexyl) carbamate (M). To a solution of (1 S,3S,5S)-N-((4-carbamimidoylthiazol-2-yl)methyl)-5-methyl-2-azabicyclo [3.1.0]hexane-3-carboxamide (L; 23 mg, 0.083 mmol) and N-(tert-butoxycarbonyl)-N-((2S,3S, 4S,5S)-2,3,4,5-tetrahydroxyhexyl)glycine (C; 26.7 mg, 0.083 mmol) in DMF (2 mL) was added DIPEA (53.3 mg, 0.413 mmol) and T₃P (187.8 mg, 0.165 mmol, 50% in ethyl acetate). The mixture was stirred under N₂ atmosphere at room temperature overnight. The reaction was quenched with saturated aq. NH₄Cl solution. The resulting mixture was extracted with ethyl acetate (3×20 mL). The combined organic layers were washed with water (2×20 mL) and brine (2×20 mL), dried over anhydrous Na₂SO₄, filtered, and concentrated to dryness under reduced pressure. The residue was purified by prep-HPLC to give the tert-butyl (2-((1 S,3S,5S)-3-(((4-carbamimidoylthiazol-2-yl)methyl)carbamoyl)-5-methyl-2-azabicyclo[3.1.0]-hexan-2-yl)-2-oxoethyl)((2S,3S,4S,5S)-2,3,4,5-tetrahydroxyhexyl)carbamate (M; 15 mg, 30.0% yield) as white solid. LC/MS (ESI) m/z: 585 (M+H)⁺.

Step 11: (1S,3S,5S)-N-((4-carbamimidoylthiazol-2-yl)methyl)-5-methyl-2-(((2S,3S,4S,5S)-2,3,4,5-tetrahydroxyhexyl)glycyl)-2-azabicyclo[3.1.0]hexane-3-carboxamide (Compound 78). A mixture of tert-butyl (2-((1 S,3S,5S)-3-(((4-carbamimidoylthiazol-2-yl)methyl)carbamoyl)-5-methyl-2-azabicyclo[3.1.0]hexan-2-yl)-2-oxoethyl)((2S,3S,4S,5S)-2,3,4,5-tetrahydroxyhexyl) carbamate (M; 15 mg, 0.026 mmol) and HCl/1,4-dioxane (1 mL) was stirred at room temperature for 1 hour. The reaction mixture was concentrated to dryness under reduced pressure. The residue was purified by prep-HPLC to give Compound 78 (5.5 mg, 44.2% yield) as a white solid. ¹H NMR (400 MHz, CD₃OD) δ 8.62 (d, J=7.5 Hz, 1H), 4.89 (d, J=3.5 Hz, 1H), 4.71 (s, 2H), 4.30 (d, J=16.1 Hz, 1H), 4.10 (d, J=16.1 Hz, 1H), 3.96 (td, J=7.7, 3.8 Hz, 1H), 3.84 (d, J=6.3 Hz, 1H), 3.79 (dd, J=13.3, 6.9 Hz, 1H), 3.48-3.42 (m, 2H), 3.28-3.19 (m, 2H), 2.51 (t, J=12.6 Hz, 1H), 2.17 (dd, J=13.4, 3.4 Hz, 1H), 1.31 (s, 4H), 1.29 (d, J=6.3 Hz, 3H), 0.84 (t, J=5.4 Hz, 1H); LC/MS (ESI) m/z: 485 (M+H)⁺.

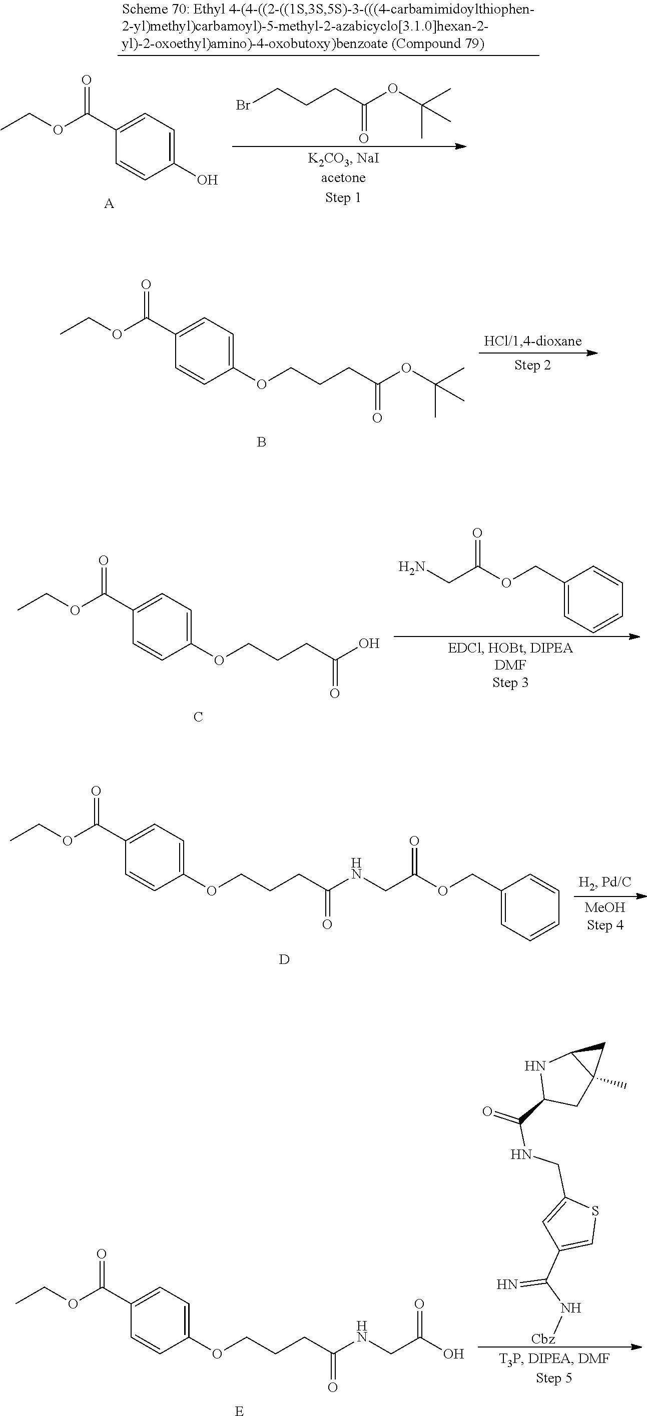

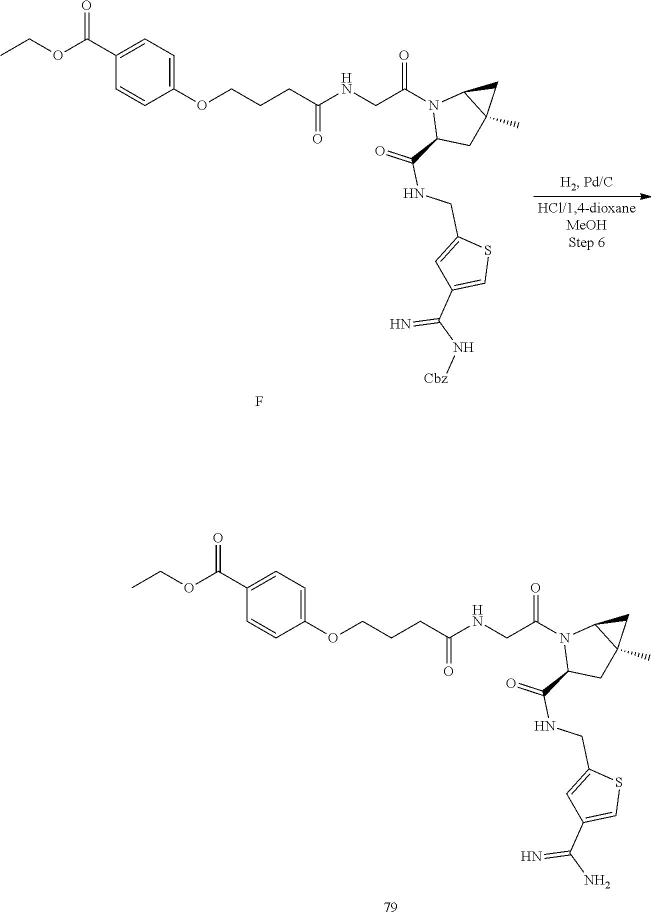

Step 1: ethyl 4-(4-(tert-butoxy)-4-oxobutoxy)benzoate (B). To a mixture of compound A (1 g, 6.02 mmol) and tert-butyl 4-bromobutanoate (1.34 g, 6.02 mmol) in acetone (30 mL) was added K₂CO₃ (2.49 g, 18.1 mmol) and NaI (225 mg, 1.51 mmol). The reaction was stirred at 60° C. for 16 hours, diluted with EtOAc, and washed with saturated aq. NH₄Cl solution. The aqueous layer was extracted with EtOAc twice, and the combined organic layers were washed with brine, dried over anhydrous Na₂SO₄, filtered, and concentrated to dryness under reduced pressure. The residue was purified by flash chromatography on silica gel (PE:EtOAc=1:1) to give compound B (775 mg, yield 41.8%) as a white solid.

Step 2: 4-(4-(ethoxycarbonyl)phenoxy)butanoic acid (C). To a solution of compound B (775 mg, 2.52 mmol) in 1,4-dioxane (7 mL) was added HCl/1,4-dioxane (7 mL) at 0° C., and the reaction was stirred at room temperature for 2 hours. The mixture was concentrated to dryness under reduced pressure, co-evaporated with DCM twice, and dried under vacuum to give compound C (580 mg, yield 91.3%) as a white solid, which was used directly in the next step without further purification. LC/MS (ESI) m/z: 251 (M−H)⁻.

Step 3: ethyl 4-(4-((2-(benzyloxy)-2-oxoethyl)amino)-4-oxobutoxy)benzoate (D). To a mixture of compound C (580 mg, 2.30 mmol) and benzyl 2-aminoacetate (569 mg, 3.45 mmol) in DMF (10 ml) was added DIPEA (1.90 mL, 11.5 mmol), EDCI (882 mg, 4.60 mmol), and HOBt (467 mg, 3.46 mmol) at room temperature, and the mixture was stirred under N₂ atmosphere at room temperature for 16 hours. The mixture was diluted with EtOAc and washed with saturated aq. NH₄Cl solution. The aqueous layer was extracted with EtOAc twice, and the combined organic layers were washed with brine, dried over anhydrous Na₂SO₄, filtered, and concentrated to dryness under reduced pressure. The residue was purified by flash chromatography on silica gel (PE:EtOAc=1:1) to give compound D (860 mg, yield 93.7%) as a white solid. LC/MS (ESI) m/z: 400 (M+H)⁺.

Step 4: (4-(4-(ethoxycarbonyl)phenoxy)butanoyl)glycine (E). To a solution of compound D (860 mg, 2.16 mmol) in MeOH (10 mL) was added Pd/C (100 mg, 10% wt.) at room temperature under N₂ atmosphere, and the reaction was stirred under a H₂ balloon at room temperature for 16 hours. The mixture was filtered, and the filtrate was concentrated to dryness under reduced pressure to give compound E (640 mg, yield 95.9%) as a white solid. LC/MS (ESI) m/z: 310 (M+H)⁺.

Step 5: ethyl 4-(4-((2-((1S,3S,5S)-3-(((4-(N-((benzyloxy)carbonyl)carbamimidoyl)thiophen-2-yl)methyl)carbamoyl)-5-methyl-2-azabicyclo[3.1.0]hexan-2-yl)-2-oxoethyl)amino)-4-oxobutoxy)benzoate (F). To a mixture of compound E (75 mg, 0.24 mmol) and benzyl (imino(5-(((1S,3S,5S)-5-methyl-2-azabicyclo[3.1.0]hexane-3-carboxamido)methyl)thiophen-3-yl)methyl)carbamate (100 mg, 0.24 mmol) in DMF (2.0 mL) was added DIPEA (0.24 mL, 1.44 mmol) and T₃P (458 mg, 0.72 mmol, 50% wt. in EtOAc) at 0° C., and the mixture was stirred under N₂ atmosphere at 25° C. for 16 hours. The mixture was diluted with water and extracted with EtOAc twice. The combined organic layers were dried over anhydrous Na₂SO₄, filtered, and concentrated to dryness under reduced pressure. The residue was purified by flash chromatography on silica gel (DCM:MeOH=12:1) to give compound F (145 mg, yield 85.9%) as a yellow oil. LC/MS (ESI) (m/z): 704 (M+H)⁺.

Step 6: Ethyl 4-(4-((2-((1S,3S,5S)-3-(((4-carbamimidoylthiophen-2-yl)methyl)carbamoyl)-5-methyl-2-azabicyclo[3.1.0]hexan-2-yl)-2-oxoethyl)amino)-4-oxobutoxy)benzoate (Compound 79). To a solution of compound F (70 mg, 0.01 mmol) in MeOH (2.0 mL) was added Pd/C (20 mg, 10% wt.) and HCl/1,4-dioxane (cat., 2 drops) at 25° C. under N₂ atmosphere, and the mixture was stirred under a H₂ balloon at 25° C. for 16 hours. The mixture was filtered, and the filtrate was concentrated to dryness under reduced pressure. The residue was purified by prep-HPLC to give Compound 79 (2.0 mg, yield 4.0%) as a white solid. ¹H NMR (400 MHz, CD₃OD) δ 8.22 (s, 1H), 7.99-7.91 (m, 2H), 7.41 (t, J=9.2 Hz, 1H), 7.01-6.95 (m, 2H), 4.81-4.78 (m, 1H), 4.51 (q, J=15.8 Hz, 2H), 4.32 (q, J=7.1 Hz, 2H), 4.14 (dt, J=12.4, 11.5 Hz, 4H), 2.47 (t, J=7.3 Hz, 2H), 2.38 (t, J=12.4 Hz, 1H), 2.18-2.05 (m, 3H), 1.36 (t, J=7.1 Hz, 3H), 1.28 (s, 3H), 1.13 (dd, J=5.7, 2.4 Hz, 1H), 0.80 (d, J=4.9 Hz 1H); LC/MS (ESI) (m/z): 570 (M+H)⁺.

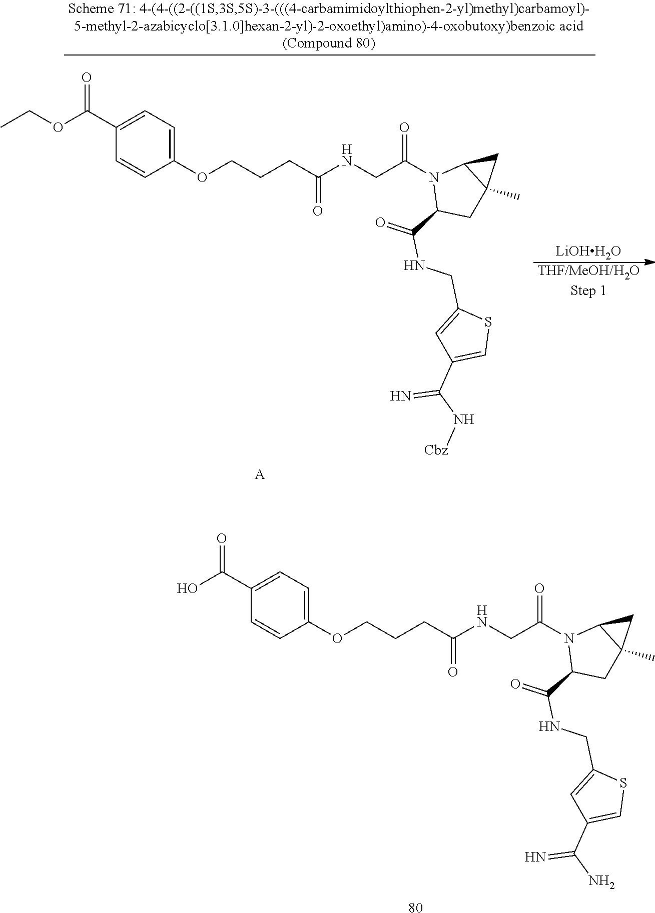

Step 1: 4-(4-((2-((1S,3S,5S)-3-(((4-carbamimidoylthiophen-2-yl)methyl)carbamoyl)-5-methyl-2-azabicyclo[3.1.0]hexan-2-yl)-2-oxoethyl)amino)-4-oxobutoxy)benzoic acid (Compound 80). To a solution of compound A (70 mg, 0.01 mmol) in THE (1.0 mL), MeOH (0.5 mL) and H₂O (0.5 mL) was added LiOH·H₂O (8.4 mg, 0.2 mmol) at 0° C., and the mixture was stirred at 25° C. for 4 hours. The mixture was diluted with H₂O and washed with EtOAc twice. The aqueous layer was acidified with 0.5 N aq. HCl solution and extracted with EtOAc twice. The combined organic layers were dried over anhydrous Na₂SO₄, filtered, and concentrated to dryness under reduced pressure. The residue was purified by prep-HPLC to give Compound 80 (1.7 mg, yield 2.5%) as a white solid. ¹H NMR (400 MHz, CD₃OD) δ 8.21 (s, 1H), 8.01-7.89 (m, 2H), 7.40 (s, 1H), 6.97 (d, J=8.9 Hz, 2H), 4.79-4.77 (m, 1H), 4.57-4.44 (m, 2H), 4.23-4.08 (m, 4H), 3.35-3.32 (m, 1H), 3.35-3.33 (m, 1H), 2.48 (t, J=7.3 Hz, 2H), 2.38 (t, J=11.7 Hz, 1H), 2.18 (dd, J=12.3, 4.7 Hz, 1H), 2.14-2.08 (m, 2H), 1.28 (s, 3H), 1.12 (d, J=3.1 Hz, 1H), 0.80 (t, J=5.3 Hz, 1H); LC/MS (ESI) (m/z): 542 (M+H)⁺.

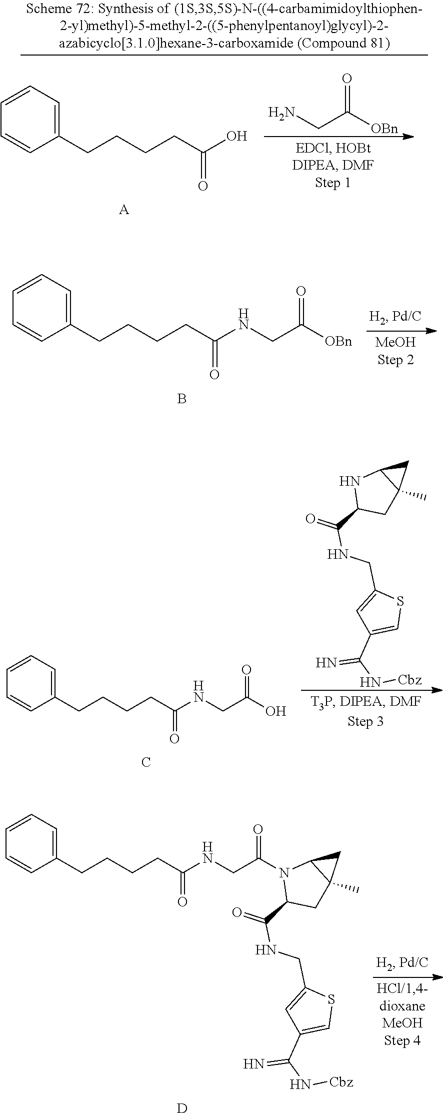

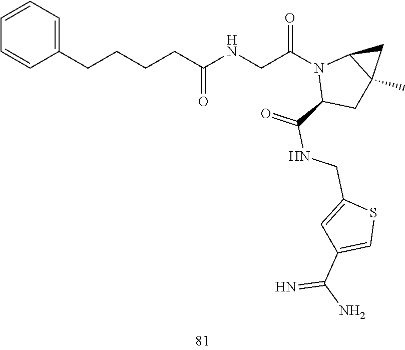 [SMILES: C[C@@]12C[C@@H]1N(C(=O)CNC(=O)CCCCc1ccccc1)[C@H](C(=O)NCc1cc(C(=N)N)cs1)C2]

Step 1: benzyl 2-(5-phenylpentanamido)acetate (B). To a mixture of compound A (500 mg, 2.81 mmol) and benzyl 2-aminoacetate (695 mg, 4.21 mmol) in DMF (10 mL) was added DIPEA (1.8 g, 14.0 mmol), EDCI (1.1 g, 5.61 mmol), and HOBt (568 mg, 4.21 mmol) at room temperature, and the mixture was stirred under N₂ atmosphere at room temperature for 3 hours. The mixture was diluted with EtOAc and washed with saturated aq. NH₄Cl solution. The aqueous layer was extracted with EtOAc twice, and the combined organic layers were washed with brine, dried over anhydrous Na₂SO₄, filtered, and concentrated to dryness under reduced pressure. The residue was purified by flash chromatography on silica gel (PE:EtOAc=3:1) to give compound B (910 mg, yield 99.7%) as a white solid. LC/MS (ESI) m/z: 326 (M+H)⁺.

Step 2: (5-phenylpentanoyl)glycine (C). To a solution of compound B (910 mg, 2.80 mmol) in MeOH (10 mL) was added Pd/C (200 mg, 10% wt) at room temperature under N₂ atmosphere, and the reaction was stirred under a H₂ balloon at room temperature for 3 hours. The mixture was filtered, and the filtrate was concentrated to dryness under reduced pressure to give compound C (640 mg, yield 97.3%) as a white solid, which was used directly in the next step without further purification. LC/MS (ESI) m/z:236 (M+H)⁺.

Step 3: benzyl (imino(5-(((1S,3S,5S)-5-methyl-2-((5-phenylpentanoyl)glycyl)-2-azabicyclo[3.1.0]hexane-3-carboxamido)methyl)thiophen-3-yl)methyl)carbamate (D). To a mixture of compound C (57 mg, 0.12 mmol) and benzyl (imino(5-(((1S,3S,5S)-5-methyl-2-azabicyclo[3.1.0]hexane-3-carboxamido)methyl)thiophen-3-yl)methyl)carbamate (100 mg, 0.12 mmol) in DMF (10 mL) was added DIPEA (94 mg, 0.73 mmol) and T₃P (231 mg, 0.36 mmol, 50% wt. in EtOAc) at 0° C., and the mixture was stirred under N₂ atmosphere at room temperature for 3 hours. The mixture was diluted with EtOAc and washed with saturated aq. NaHCO₃ solution. The aqueous layer was extracted with EtOAc twice and the combined organic layers were washed with brine, dried over anhydrous Na₂SO₄, filtered, and concentrated to dryness under reduced pressure. The residue was purified by flash chromatography on silica gel (PE:EtOAc=1:1) to give compound D (70 mg, yield 91.7%) as a white solid. LC/MS (ESI) m/z: 630 (M+H)⁺.

Step 4: (1S,3S,5S)-N-((4-carbamimidoylthiophen-2-yl)methyl)-5-methyl-2-((5-phenyl-pentanoyl)glycyl)-2-azabicyclo[3.1.0]hexane-3-carboxamide (Compound 81). To a solution of compound D (70 mg, 0.11 mmol) in MeOH (5 mL) was added Pd/C (80 mg, 10% wt) and HCl/1,4-dioxane (cat., 2 drops) at room temperature under N₂ atmosphere, and the reaction was stirred under a H₂ balloon at room temperature for 16 hours. The mixture was filtered, and the filtrate was concentrated to dryness under reduced pressure. The residue was purified by prep-HPLC to give Compound 81 (20 mg, yield 36.3%) as a white solid. ¹H NMR (400 MHz, CD₃OD) δ 8.52 (s, 1H), 8.22 (d, J=1.4 Hz, 1H), 7.41 (s, 1H), 7.25-7.21 (m, 2H), 7.18-7.12 (m, 3H), 4.79 (d, J=8.1 Hz, 1H), 4.49 (t, J=12.7 Hz, 2H), 4.15 (q, J=16.7 Hz, 2H), 3.35-3.32 (m, 1H), 2.62 (t, J=7.0 Hz, 2H), 2.42-2.35 (m, 1H), 2.28 (d, J=7.0 Hz, 2H), 2.16 (dd, J=13.3, 3.1 Hz, 1H), 1.70-1.58 (m, 4H), 1.27 (s, 3H), 1.13-1.09 (m, 1H), 0.78 (t, J=5.3 Hz, 1H); LC/MS (ESI) m/z: 496 (M+H)⁺.

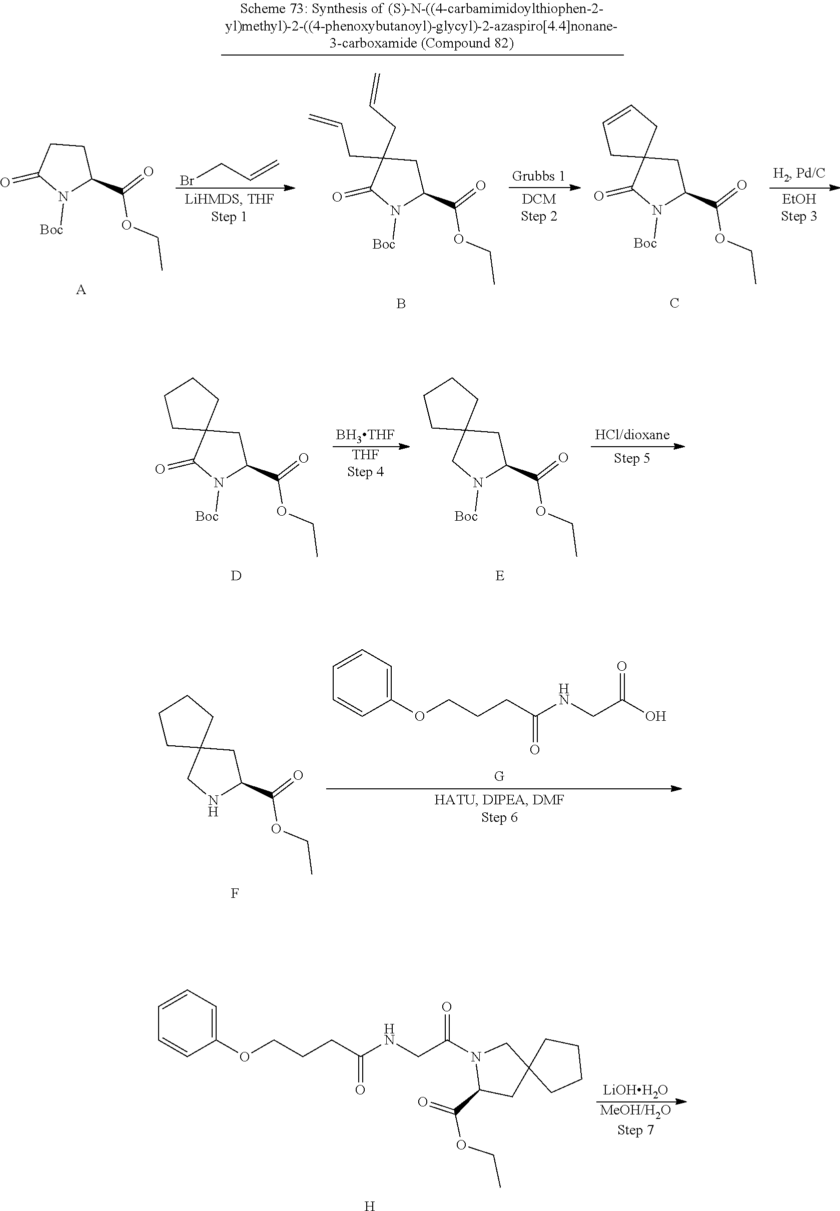

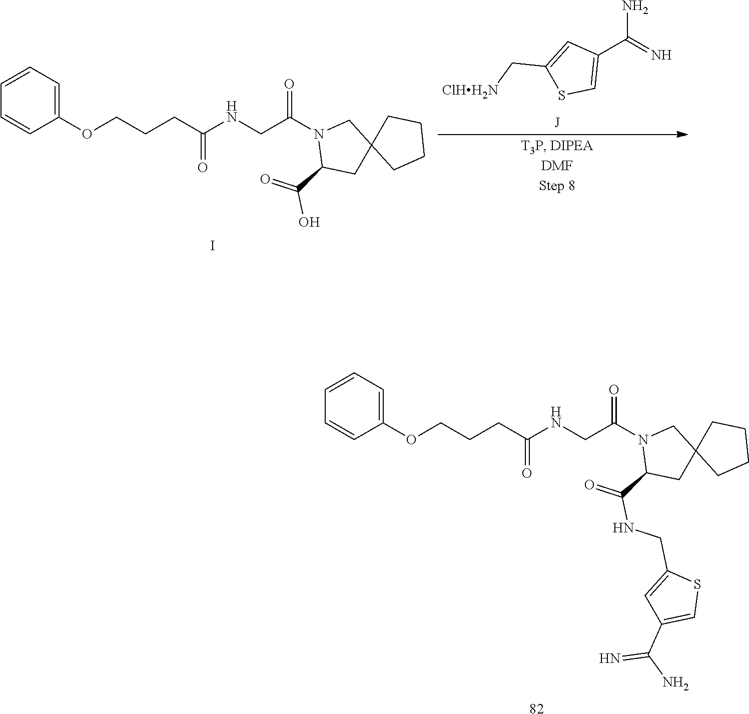

Step 1: 1-(tert-butyl) 2-ethyl (S)-4,4-diallyl-5-oxopyrrolidine-1,2-dicarboxylate (B). To a solution of 1-(tert-butyl) 2-ethyl (S)-5-oxopyrrolidine-1,2-dicarboxylate (A; 2.0 g, 7.77 mmol) in THE (100 mL) was added LiHMDS (15.5 mL, 15.5 mmol, 1 mol in THF) dropwise at −70° C. The reaction mixture was stirred at −70° C. for 1 hour before the addition of allyl bromide (1.88 g, 15.5 mmol). The mixture was stirred at room temperature for 2 hours before quenched with diluted acetic acid (1.2 mL in 5 mL of water). The reaction was extracted with ethyl acetate (3×30 mL). The combined organic layers were washed with water (2×20 mL) and brine (2×20 mL), dried over Na₂SO₄, filtered, and concentrated to dryness under reduced pressure. The residue was purified by flash chromatography on silica gel (petroleum ether:ethyl acetate=10:1 to 5:1) to give 1-(tert-butyl) 2-ethyl (S)-4,4-diallyl-5-oxopyrrolidine-1,2-dicarboxylate (B; 0.62 g, 23.6% yield) as a colorless oil. LC/MS (ESI) (m/z): 338 (M+H)⁺.

Step 2: 2-(tert-butyl) 3-ethyl (S)-1-oxo-2-azaspiro[4.4]non-7-ene-2,3-dicarboxylate (C). To a solution of 1-(tert-butyl) 2-ethyl (S)-4,4-diallyl-5-oxopyrrolidine-1,2-dicarboxylate (A; 620 mg, 1.84 mmol) in dichloromethane (50 mL) was added Grubbs 1^{st} catalyst (75.6 mg, 0.092 mmol) under N₂ atmosphere. The reaction was stirred at room temperature overnight. The reaction mixture was concentrated to dryness under reduced pressure and the residue was purified by flash chromatography on silica gel (petroleum ether:ethyl acetate=30:1 to 5:1) to give 2-(tert-butyl) 3-ethyl (S)-1-oxo-2-azaspiro[4.4]non-7-ene-2,3-dicarboxylate (C; 440.0 mg, 77.4% yield) as a colorless oil. LC/MS (ESI) m/z: 310 (M+H)⁺.

Step 3: 2-(tert-butyl) 3-ethyl (S)-1-oxo-2-azaspiro[4.4]nonane-2,3-dicarboxylate (D). To a stirred solution of 2-(tert-butyl) 3-ethyl (S)-1-oxo-2-azaspiro[4.4]non-7-ene-2,3-dicarboxylate (C; 440 mg, 1.42 mmol) in ethanol (6 mL) was added Pd/C (50 mg, 10% wt), and the mixture was degassed under N₂ atmosphere for three times and stirred under a H₂ balloon at 25° C. for 2 hours. The mixture was filtered and the filtrate was concentrated to dryness under reduced pressure to give crude 2-(tert-butyl) 3-ethyl (S)-1-oxo-2-azaspiro[4.4]nonane-2,3-dicarboxylate (D; 400 mg, 90.3% yield) as a colorless oil, which was used directly in the next step without further purification. LC/MS (ESI) m/z: 312 (M+H)⁺.

Step 4: 2-(tert-butyl) 3-ethyl (S)-2-azaspiro[4.4]nonane-2,3-dicarboxylate (E). To a solution of 2-(tert-butyl) 3-ethyl (S)-1-oxo-2-azaspiro[4.4]nonane-2,3-dicarboxylate (D; 200 mg, 0.64 mmol) in THE (3 mL) was added BH₃THF (2.6 mL, 2.57 mmol, 1 M in THF) at 0° C. The mixture was stirred at 60° C. overnight under N₂ atmosphere. The reaction was cooled down to room temperature and quenched with saturated aq.NH₄Cl solution. The resulting mixture was extracted with ethyl acetate (3×10 mL). The combined organic phases were washed with water (2×10 mL) and brine (2×10 mL), dried over anhydrous Na₂SO₄, filtered and concentrated to dryness under reduced pressure. The residue was purified by flash chromatography on silica gel (petroleum ether:ethyl acetate=20:1 to 10:1) to give 2-(tert-butyl) 3-ethyl (S)-2-azaspiro[4.4]nonane-2,3-dicarboxylate (110 mg, 57.6% yield) as a light-yellow oil. LC/MS (ESI) m/z: 298 (M+H)⁺.

Step 5: 2-(tert-butyl) 3-ethyl (S)-2-azaspiro[4.4]nonane-2,3-dicarboxylate (F). A mixture of 2-(tert-butyl) 3-ethyl (S)-2-azaspiro[4.4]nonane-2,3-dicarboxylate (E; 110 mg, 0.37 mmol) and HCl/1,4-dioxane (2 mL, 4 M) was stirred at room temperature for 1 hour. The reaction mixture was concentrated to dryness under reduced pressure, co-evaporated with dichloromethane and dried under vacuum to give 2-(tert-butyl) 3-ethyl (S)-2-azaspiro[4.4]nonane-2,3-dicarboxylate (F; 74 mg, quantitative yield) as a yellow solid, which was used directly in the next step without further purification. LC/MS (ESI) m/z: 198 (M+H)⁺.

Step 6: ethyl (S)-2-((4-phenoxybutanoyl)glycyl)-2-azaspiro[4.4]nonane-3-carboxylate (H). To a mixture of 2-(tert-butyl) 3-ethyl (S)-2-azaspiro[4.4]nonane-2,3-dicarboxylate (F; 74 mg, 0.37 mmol) and (4-phenoxybutanoyl)glycine (G; 88 mg, 0.37 mmol) in DMF (3 mL) was added HATU (171 mg, 0.45 mmol) and DIPEA (145 mg, 1.12 mmol). The mixture was stirred at room temperature under N₂ atmosphere overnight. The reaction was quenched with saturated aq. NH₄Cl solution. The resulting mixture was extracted with ethyl acetate (3×20 mL). The combined organic layers were washed with water (2×20 mL) and brine (2×20 mL), dried over anhydrous Na₂SO₄, filtered and concentrated to dryness under reduced pressure. The residue was purified by flash chromatography on silica gel (petroleum ether:ethyl acetate=5:1 to 1:1) to give ethyl (S)-2-((4-phenoxybutanoyl)glycyl)-2-azaspiro[4.4]nonane-3-carboxylate (H; 140 mg, 89.6% yield) as a light-yellow oil. LC/MS (ESI) m/z: 417 Step 7: (S)-2-((4-phenoxybutanoyl)glycyl)-2-azaspiro[4.4]nonane-3-carboxylic acid (1). To a solution of ethyl (S)-2-((4-phenoxybutanoyl)glycyl)-2-azaspiro[4.4]nonane-3-carboxylate (H; 140 mg, 0.34 mmol) in methanol (1.5 mL) and water (0.5 mL) was added a solution of LiOH·H₂O (17 mg, 0.40 mmol) at 0° C. and the mixture was stirred at room temperature for 2 hours. The mixture was acidified with 1 N aq. HCl solution to pH<3 and extracted with dichloromethane (2×10 mL). The combined organic layers were concentrated to dryness under reduced pressure to give (S)-2-((4-phenoxybutanoyl)glycyl)-2-azaspiro[4.4]nonane-3-carboxylic acid (125 mg, 95.7% yield) as a white solid, which was used directly in next step without further purification. LC/MS (ESI) (m/z): 389 (M+H)⁺.

Step 8: (S)-N-((4-carbamimidoylthiophen-2-yl)methyl)-2-((4-phenoxybutanoyl)glycyl)-2-azaspiro[4.4]nonane-3-carboxamide (Compound 82). To a solution of (S)-2-((4-phenoxybutanoyl)glycyl)-2-azaspiro[4.4]nonane-3-carboxylic acid (I; 45 mg, 0.11 mmol) and 5-(aminomethyl)thiophene-3-carboximidamide hydrochloride (31 mg, 0.16 mmol) in DMF (2 mL) was added DIPEA (42 mg, 0.32 mmol) and T₃P (137 mg, 0.22 mmol, 50% in ethyl acetate). The mixture was stirred at room temperature under N₂ atmosphere overnight. The reaction was quenched with saturated aq. NH₄Cl solution. The resulting mixture was extracted with ethyl acetate (3×20 mL). The combined organic layers were washed with water (2×20 mL) and brine (2×20 mL), dried over anhydrous Na₂SO₄, filtered and concentrated to dryness under reduced pressure. The residue was purified by prep-HPLC to give Compound 82 (6.4 mg, 11.3% yield) as a white solid. ¹H NMR (400 MHz, CD₃OD) δ 8.24 (d, J=5.8 Hz, 1H), 7.47 (d, J=18.4 Hz, 1H), 7.28-7.22 (m, 2H), 6.94-6.88 (m, 3H), 4.63-4.55 (m, 2H), 4.42 (t, J=7.9 Hz, 1H), 4.09-3.92 (m, 4H), 3.51 (dd, J=31.7, 9.9 Hz, 2H), 2.51-2.45 (m, 2H), 2.19 (dd, J=12.5, 8.3 Hz, 1H), 2.07 (dd, J=14.1, 7.0 Hz, 2H), 1.91 (dd, J=12.5, 7.7 Hz, 1H), 1.71-1.64 (m, 4H), 1.63-1.51 (m, 3H), 1.05 (t, J=7.2 Hz, 1H); LC/MS(ESI) m/z: 526 (M+H)⁺.

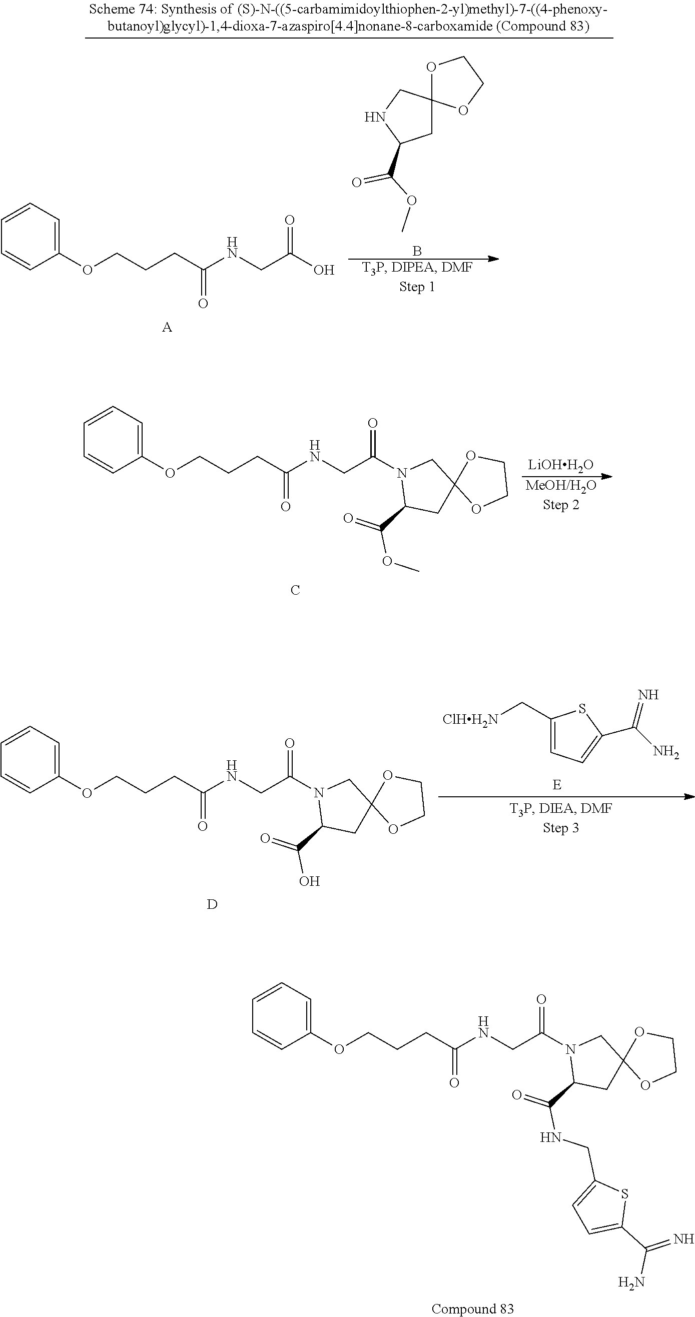

Step 1: methyl (S)-7-((4-phenoxybutanoyl)glycyl)-1,4-dioxa-7-azaspiro[4.4]nonane-8-carboxylate (C). To a solution of 4-phenoxybutanoyl)glycine (A; 200 mg, 0.84 mmol) and methyl (S)-1,4-dioxa-7-azaspiro[4.4]nonane-8-carboxylate (B; 237 mg, 1.26 mmol) in DMF (3 mL) was added DIPEA (326 mg, 2.53 mmol) and T₃P (1.07 g, 1.69 mmol, 50% in ethyl acetate). The mixture was stirred at room temperature under N₂ atmosphere overnight. The reaction was quenched with saturated aq.NH₄Cl solution. The resulting mixture was extracted with ethyl acetate (3×20 mL). The combined organic layers were washed with water (2×20 mL) and brine (2×20 mL), dried over anhydrous Na₂SO₄, filtered and concentrated to dryness under reduced pressure. The residue was purified by flash chromatography on silica gel (dichloromethane:methanol=100:1 to 15:1) to give methyl (S)-7-((4-phenoxybutanoyl)glycyl)-1,4-dioxa-7-azaspiro[4.4]nonane-8-carboxylate (C; 320 mg, 93.4% yield) as yellow oil. LC/MS (ESI) m/z: 407 (M+H)⁺.

Step 2: (S)-7-((4-phenoxybutanoyl)glycyl)-1,4-dioxa-7-azaspiro[4.4]nonane-8-carboxylic acid (D). To a solution of methyl (S)-7-((4-phenoxybutanoyl)glycyl)-1,4-dioxa-7-azaspiro[4.4]nonane-8-carboxylate (C; 320 mg, 0.79 mmol) in methanol (1.5 mL) and water (0.5 mL) was added a solution of LiOH·H₂O (33 mg, 0.79 mmol) in water (0.5 mL) at 0° C. and the mixture was stirred at room temperature for 16 hours. The mixture was acidified with 1 N aq. HCl solution to pH˜3 and extracted with dichloromethane (2×10 mL). The combined organic layers were concentrated to dryness under reduced pressure to give (S)-7-((4-phenoxybutanoyl)glycyl)-1,4-dioxa-7-azaspiro[4.4]nonane-8-carboxylic acid (D; 300 mg, 97.1% yield) as a yellow oil, which was used directly in next step without further purification. LC/MS (ESI) (m/z): 393 (M+H)⁺.

Step 3: (S)-N-((5-carbamimidoylthiophen-2-yl)methyl)-7-((4-phenoxybutanoyl)glycyl)-1,4-dioxa-7-azaspiro[4.4]nonane-8-carboxamide (Compound 83). To a solution of (S)-7-((4-phenoxybutanoyl)glycyl)-1,4-dioxa-7-azaspiro[4.4]nonane-8-carboxylic acid (D; 40 mg, 0.10 mmol) and 5-(aminomethyl)thiophene-2-carboximidamide hydrochloride (E; 29 mg, 0.15 mmol) in DMF (3 mL) was added DIPEA (66 mg, 0.51 mmol) and T₃P (195 mg, 0.30 mmol, 50% in ethyl acetate). The mixture was stirred at room temperature under N₂ atmosphere overnight. The reaction was quenched with saturated aq.NH₄Cl solution. The resulting mixture was extracted with ethyl acetate (3×20 mL). The combined organic layers were washed with water (2×20 mL) and brine (2×20 mL), dried over anhydrous Na₂SO₄, filtered and concentrated to dryness under reduced pressure. The residue was purified by prep-HPLC to give Compound 83 (2.7 mg, 5.0% yield) as a white solid. ¹H NMR (400 MHz, CD₃OD) δ 8.51 (s, 1H), 7.78 (dd, J=6.4, 4.0 Hz, 1H), 7.25-7.16 (m, 3H), 6.92-6.88 (m, 3H), 4.61 (d, J=1.2 Hz, 2H), 4.58-4.52 (m, 1H), 4.01-3.94 (m, 8H), 3.70 (d, J=7.7 Hz, 2H), 2.48-2.41 (m, 3H), 2.22-2.15 (m, 1H), 2.14 (m, 2H); LC/MS (ESI) m/z: 530 (M+H)⁺.

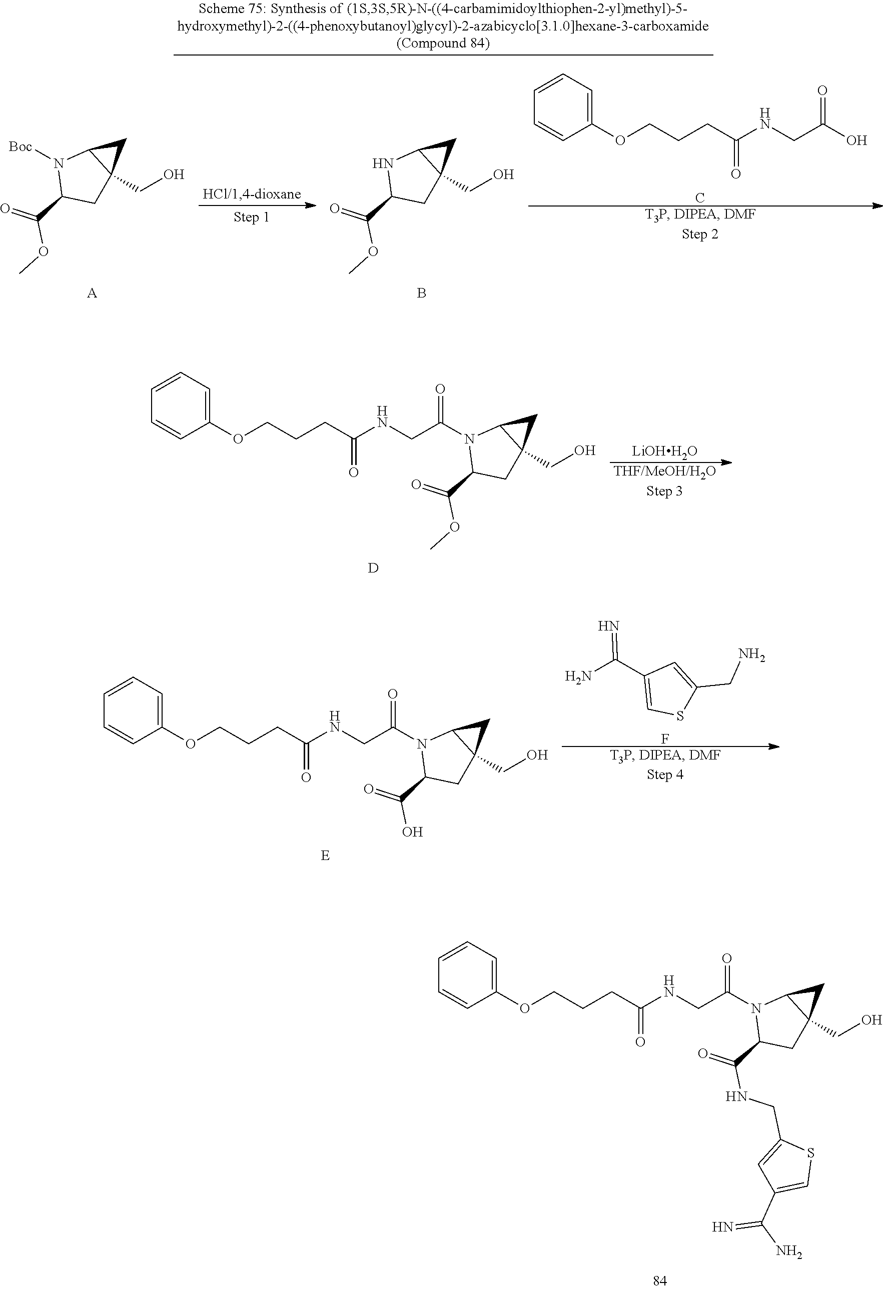

Step 1: methyl (1S,3S,5R)-5-(hydroxymethyl)-2-azabicyclo[3.1.0]hexane-3-carboxylate (B). A mixture of compound A (58 mg, 0.21 mmol) and HCl/1,4-dioxane (5 mL) was stirred at room temperature for 1 hour. The reaction was concentrated to dryness under reduced pressure, co-evaporated with DCM twice and dried under vacuum to give compound B (36 mg, yield 98.4%) as a colorless oil, which was used directly in the next step. LC/MS (ESI) m/z: 172 (M+H)⁺.

Step 2: methyl (1S,3S,5R)-5-(hydroxymethyl)-2-((4-phenoxybutanoyl)glycyl)-2-azabicyclo-[3.1.0]hexane-3-carboxylate (D). To a mixture of compound B (36 mg, 0.21 mmol) and (4-phenoxybutanoyl)glycine (C; 56 mg, 0.23 mmol) in DMF (3 mL) was added DIPEA (0.2 mL, 1.26 mmol) and T₃P (413 mg, 0.65 mmol, 50% wt in EtOAc) at 0° C. and the mixture was stirred under N₂ atmosphere at 25° C. for 16 hours. The mixture was diluted with EtOAc and washed with saturated aq. NaHCO₃ solution. The aqueous layer was extracted with EtOAc twice. The combined organic layers were dried over anhydrous Na₂SO₄, filtered and concentrated to dryness under reduced pressure. The residue was purified by flash chromatography on silica gel (DCM:MeOH=30:1) to give compound D (70 mg, yield 85.4%) as a yellow solid. LC/MS (ESI) m/z: 391 (M+H)⁺.

Step 3: (1S,3S,5R)-5-(hydroxymethyl)-2-((4-phenoxybutanoyl)glycyl)-2-azabicyclo[3.1.0]-hexane-3-carboxylic acid (E). To a solution of compound D (70 mg, 0.18 mmol) in MeOH (2 mL), THE (1 mL) and H₂O (1 mL) was added LiOH·H₂O (11 mg, 0.27 mmol) at 0° C. and the mixture was stirred at room temperature for 2 hours. The mixture was diluted with water and washed with EtOAc. The aqueous layer was acidified with 1 N aq. HCl solution to pH-3 and extracted with CHCl₃/i-PrOH (v/v=3/1) five times. The combined organic layers were dried over anhydrous Na₂SO₄, filtered and concentrated to dryness under reduced pressure to give compound E (60 mg, yield 89.6%) as a light-yellow oil, which was used directly in the next step. LC/MS (ESI) (m/z): 377 (M+H)⁺.

Step 4: (1S,3S,5R)-N-((4-carbamimidoylthiophen-2-yl)methyl)-5-(hydroxymethyl)-2-((4-phenoxybutanoyl)glycyl)-2-azabicyclo[3.1.0]hexane-3-carboxamide (Compound 84). To a mixture of compound E (60 mg, 0.16 mmol) and 5-(aminomethyl)thiophene-3-carboximidamide (37 mg, 0.24 mmol) in DMF (5 mL) was added DIPEA (0.16 mL, 0.96 mmol) and T₃P (304 mg, 0.48 mmol, 50% wt in EtOAc) at 0° C. and the mixture was stirred under N₂ atmosphere at 25° C. for 16 hours. The mixture was diluted with saturated aq. NaHCO₃ solution and extracted with CHCl₃/i-PrOH (v/v=3/1) five times. The combined organic layers were dried over anhydrous Na₂SO₄, filtered and concentrated to dryness under reduced pressure. The residue was purified by flash chromatography on silica gel (DCM:MeOH=5:1) and further purified by prep-HPLC to give Compound 84 (10.0 mg, yield 12.2%) as a white solid. ¹H NMR (400 MHz, CD₃OD) δ 8.53 (s, 1H), 8.22 (s, 1H), 7.42 (s, 1H), 7.26-7.22 (m, 2H), 6.91-6.88 (m, 3H), 4.83-4.81 (m, 1H), 4.58-4.47 (m, 2H), 4.24-4.11 (m, 2H), 4.17 (t, J=6.0 Hz, 2H), 3.68-3.63 (m, 2H), 3.51-3.46 (m, 2H), 2.65 (t, J=12.3 Hz, 1H), 2.47 (t, J=7.4 Hz, 2H), 2.14-2.02 (m, 3H), 1.24-1.19 (m, 1H), 1.02-0.97 (m, 1H); LC/MS (ESI) m/z: 514 (M+H)⁺.

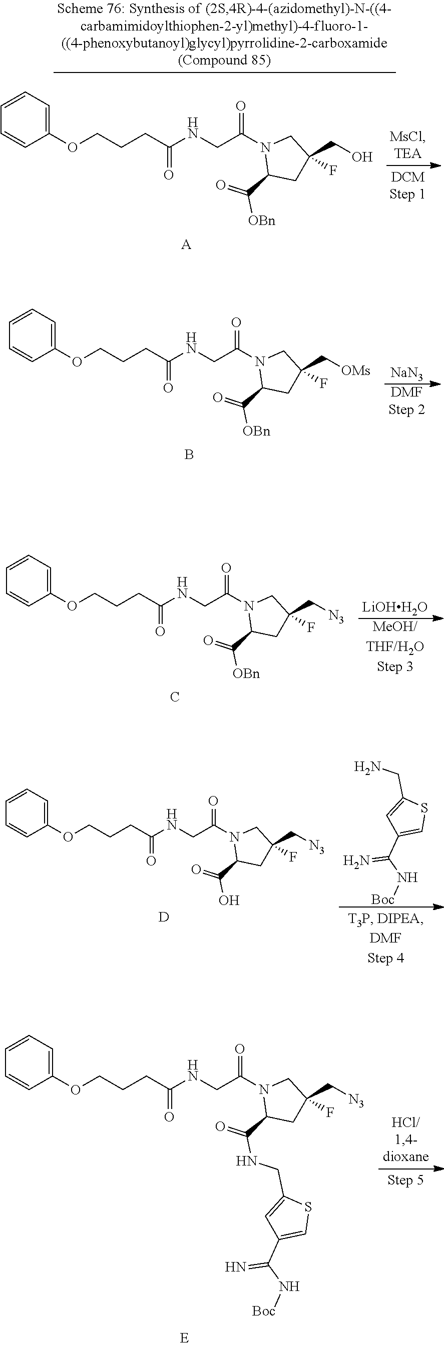

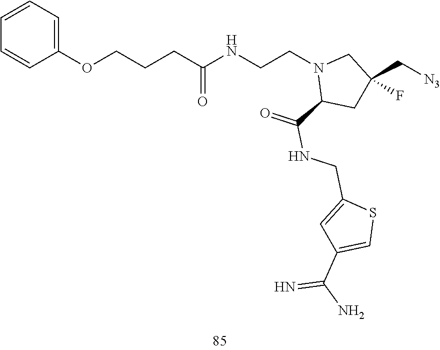 [SMILES: [N-]=[N+]=NC[C@@]1(F)C[C@@H](C(=O)NCc2cc(C(=N)N)cs2)N(CCNC(=O)CCCOc2ccccc2)C1]

Step 1: benzyl (2S,4R)-4-fluoro-4-(((methylsulfonyl)oxy)methyl)-1-((4-phenoxybutanoyl)-glycyl)pyrrolidine-2-carboxylate (B). To a solution of compound A (190 mg, 0.40 mmol) in DCM (6 mL) was added TEA (244 mg, 2.42 mmol) and MsCl (139 mg, 1.21 mmol) at 0° C. and the reaction was stirred under N₂ atmosphere at 0° C. for 2 hours. The mixture was quenched with ice-water and extracted with DCM twice. The combined organic layers were washed with brine, dried over anhydrous Na₂SO₄, filtered and concentrated to dryness under reduced pressure to give compound B (200 mg, yield 90.5%) as a yellow oil, which was used directly in the next step. LC/MS(ESI) m/z: 551 (M+H)⁺.

Step 2: benzyl (2S,4R)-4-(azidomethyl)-4-fluoro-1-((4-phenoxybutanoyl)glycyl)pyrrolidine-2-carboxylate (C). To a solution of compound B (200 mg, 0.36 mmol) in DMF (9 mL) was added NaN₃ (59 mg, 0.91 mmol) at room temperature and the reaction was stirred at 55° C. for 2 days. The mixture was diluted with ice-water and extracted with EtOAc twice. The combined organic layers were washed with brine, dried over anhydrous Na₂SO₄, filtered and concentrated to dryness under reduced pressure. The residue was purified by flash chromatography on silica gel (PE:EtOAc=30:1 to 1:1) to give compound C (100 mg, yield 55.3%) as a yellow oil. LC/MS(ESI) m/z: 498 (M+H)⁺.

Step 3: (2S,4R)-4-(azidomethyl)-4-fluoro-1-((4-phenoxybutanoyl)glycyl)pyrrolidine-2-carboxylic acid (D). To a solution of compound C (100 mg, 0.20 mmol) in MeOH (1.2 mL), THE (0.6 mL) and H₂O (0.6 mL) was added LiOH·H₂O (13 mg, 0.30 mmol) at 0° C. and the mixture was stirred at room temperature for 2 hours. The mixture was diluted with water and washed with EtOAc twice. The aqueous layer was acidified with 0.5 M aq. HCl solution to pH˜3 and extracted with EtOAc twice. The combined organic layers were washed with brine, dried over anhydrous Na₂SO₄, filtered and concentrated to dryness under reduced pressure to give compound D (80 mg, yield 97.7%) as a light-yellow oil, which was used directly in the next step. LC/MS(ESI) m/z: 408 (M+H)⁺.

Step 4: tert-butyl ((5-(((2S,4R)-4-(azidomethyl)-4-fluoro-1-((4-phenoxybutanoyl)glycyl)-pyrrolidine-2-carboxamido)methyl)thiophen-3-yl)(imino)methyl)carbamate (E). To a mixture of compound D (80 mg, 0.20 mmol) and tert-butyl ((5-(aminomethyl)thiophen-3-yl)(imino)methyl)carbamate (53 mg, 0.20 mmol) in DMF (3 mL) was added DIPEA (0.22 mL, 1.2 mmol) and T₃P (382 mg, 0.6 mmol, 50% wt in EtOAc) at 0° C. and the mixture was stirred under N₂ atmosphere at 25° C. for 2 hours. The mixture was diluted with EtOAc and washed with saturated aq. NaHCO₃ solution. The aqueous layer was extracted with EtOAc twice. The combined organic layers were washed with brine, dried over anhydrous Na₂SO₄, filtered and concentrated to dryness under reduced pressure. The residue was purified by flash chromatography on silica gel (DCM:MeOH=10:1) to give compound E (77 mg, yield 57.9%) as a light-yellow oil. LC/MS (ESI) (m/z): 645 (M+H)⁺.

Step 5: (2S,4R)-4-(azidomethyl)-N-((4-carbamimidoylthiophen-2-yl)methyl)-4-fluoro-1-((4-phenoxybutanoyl)glycyl)pyrrolidine-2-carboxamide (Compound 86). A mixture of compound E (25 mg, 0.04 mmol) and HCl/1,4-dioxane (2 mL) was stirred at room temperature for 8 hours. The reaction was concentrated to dryness under reduced pressure, co-evaporated with DCM twice and dried under vacuum. The residue was purified by prep-HPLC to give Compound 85 (3.9 mg, yield 18.5%) as a white solid. ¹H NMR (400 MHz, CD₃OD) δ 8.22 (s, 1H), 7.45 (s, 1H), 7.26-7.22 (m, 2H), 6.91-6.88 (m, 3H), 4.67-4.52 (m, 3H), 4.13-3.95 (m, 5H), 3.83-3.62 (m, 3H), 2.69-2.45 (m, 3H), 2.20-2.05 (m, 3H); LC/MS (ESI) m/z: 545 (M+H)⁺.

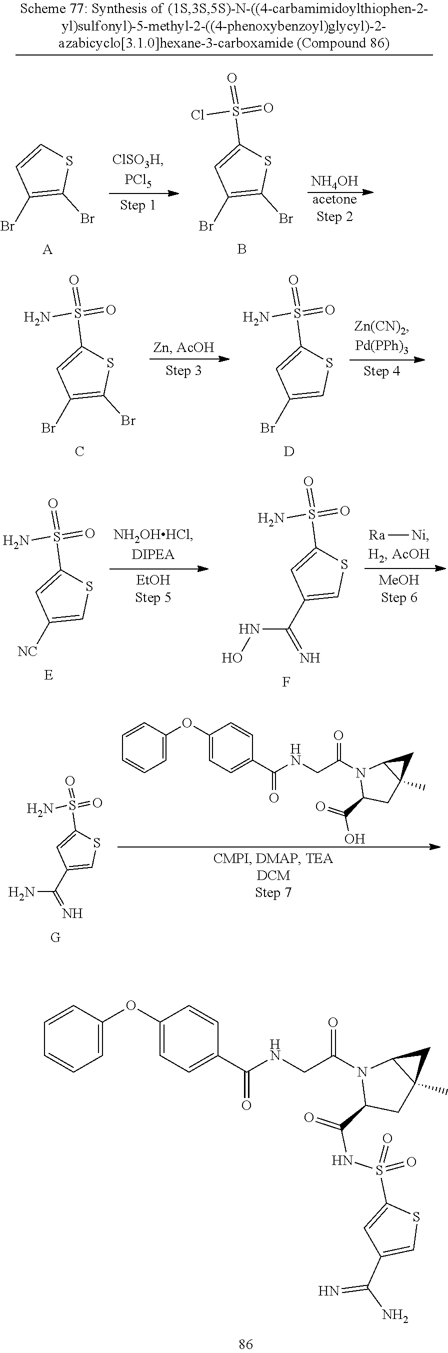

Step 1: 4,5-dibromothiophene-2-sulfonyl chloride (B). To a mixture of compound A (3 g, 12.4 mmol) and ClSO₃H (2.2 g, 18.6 mmol) was added PCl₅ (2.6 g, 12.4 mmol) at 0° C. and the mixture was stirred under N₂ atmosphere at 50° C. for 2 hours. The mixture was diluted with EtOAc and washed with saturated aq.NaHCO₃ solution. The aqueous layer was extracted with EtOAc twice and the combined organic layers were washed with brine, dried over anhydrous Na₂SO₄, filtered and concentrated to dryness under reduced pressure. The residue was purified by flash chromatography on silica gel (PE:EtOAc=5:1) to give compound B (4.2 g, yield 98.8%) as a brown solid. LC/MS (ESI) m/z: 339 (M+H)⁺.

Step 2: 4,5-dibromothiophene-2-sulfonamide (C). To a solution of compound B (4.2 g, 12.3 mmol) in acetone (60 mL) was added NH₃—H₂O (62.5 mL, 25% wt in water) at room temperature and the mixture was stirred under N₂ atmosphere at room temperature for 2 hours. The mixture was extracted with EtOAc twice and the combined organic layers were washed with brine, dried over anhydrous Na₂SO₄, filtered and concentrated to dryness under reduced pressure to give compound C (3.1 g, yield 78.3%) as a white solid, which was used directly in the next step. LC/MS (ESI) m/z: 318 (M−H)⁻.

Step 3: 4-bromothiophene-2-sulfonamide (D). To a solution of compound C (2 g, 6.23 mmol) in AcOH (3 mL) and H₂O (8 mL) was added zinc powder (0.9 g, 14.33 mmol) at room temperature and the reaction was stirred under N₂ atmosphere at 100° C. for 30 minutes. The mixture was diluted with EtOAc and washed with saturated aq. NaHCO₃ solution. The aqueous layer was extracted with EtOAc twice. The combined organic layers were washed with brine, dried over anhydrous Na₂SO₄, filtered and concentrated to dryness under reduced pressure. The residue was purified by flash chromatography on silica gel (PE:EtOAc=1:1) to give compound D (1.3 g, yield 86.2%) as a white solid. LC/MS (ESI) m/z: 240 (M−H)⁻.

Step 4: 4-cyanothiophene-2-sulfonamide (E). To a solution of compound D (1 g, 4.13 mmol) in NMP (20 mL) was added Zn(CN)₂ (970 mg, 8.26 mmol) and Pd(PPh₃)₄ (477 mg, 0.41 mmol) at room temperature and the mixture was stirred under N₂ atmosphere at 160° C. for 2 hours. The mixture was diluted with water and extracted with EtOAc twice. The combined organic layers were washed with brine, dried over anhydrous Na₂SO₄, filtered and concentrated to dryness under reduced pressure. The residue was purified by flash chromatography on silica gel (PE:EtOAc=1:1) to give compound E (210 mg, yield 27.0%) as a brown solid. LC/MS (ESI) m/z: 187 (M−H)⁻.

Step 5: N-hydroxy-5-sulfamoylthiophene-3-carboximidamide (F). To a solution of compound 5 (330 mg, 1.75 mmol) in EtOH (10 mL) was added NH₂OH HCl (0.3 g, 4.38 mmol) and DIPEA (0.7 g, 5.26 mmol) at 0° C. and the mixture was stirred under N₂ atmosphere at room temperature for 15 hours. The mixture was diluted with water and extracted with EtOAc twice. The combined organic layers were washed with brine, dried over anhydrous Na₂SO₄, filtered and concentrated to dryness under reduced pressure. The residue was purified by flash chromatography on silica gel (PE:EtOAc=1:1) to give compound 6 (380 mg, yield 98.0%) as a white solid. LC/MS (ESI) m/z: 220 (M−H)⁻.

Step 6: 5-sulfamoylthiophene-3-carboximidamide (G). To a solution of compound F (380 mg, 1.72 mmol) in MeOH (5 mL) was added Raney Ni (1 mL, slurry in water) and AcOH (0.1 mL) at room temperature under N₂ atmosphere and the reaction was stirred under a H₂ balloon at room temperature for 16 hours. The mixture was filtered and the filtrate was concentrated to dryness under reduced pressure to give compound G (340 mg, yield 96.4%) as a white solid, which was used directly in the next step. LC/MS (ESI) m/z: 206 (M+H)⁺.

Step 7: (1S,3S,5S)-N-((4-carbamimidoylthiophen-2-yl)sulfonyl)-5-methyl-2-((4-phenoxy-benzoyl)glycyl)-2-azabicyclo[3.1.0]hexane-3-carboxamide (Compound 86). To a mixture of compound G (100 mg, 0.24 mmol) and (1S,3S,5S)-5-methyl-2-((4-phenoxybenzoyl)glycyl)-2-azabicyclo[3.1.0]hexane-3-carboxylic acid (192 mg, 0.24 mmol) in DCM (5 mL) was added TEA (147 mg, 1.46 mmol), DMAP (3 mg, 0.024 mmol) and CMPI (93 mg, 0.37 mmol) at 0° C. and the mixture was stirred under N₂ atmosphere at room temperature for 48 hours. The mixture was diluted with DCM and washed with saturated aq. NH₄Cl solution. The aqueous layer was extracted with CHCl₃/i-PrOH (v/v=3/1) five times. The combined organic layers were dried over anhydrous Na₂SO₄, filtered and concentrated to dryness under reduced pressure. The residue was purified by flash chromatography on silica gel (DCM:MeOH=10:1) and further purified by prep-HPLC to give Compound 86 (1 mg, yield 0.71%) as a white solid. ¹H NMR (400 MHz, CD₃OD) δ 8.57-8.55 (m, 1H), 8.16-8.13 (m, 1H), 7.87-7.83 (m, 2H), 7.44-7.40 (m, 2H), 7.20 (t, J=7.4 Hz, 1H), 7.07-6.99 (m, 4H), 4.74-4.71 (m, 1H), 4.35-4.29 (m, 1H), 4.26-4.17 (m, 1H), 3.40-3.36 (m, 1H), 2.50-2.39 (m, 1H), 2.03-1.98 (m, 1H), 1.28 (s, 3H), 1.19-1.17 (m, 1H), 0.85-0.75 (m, 1H); LC/MS (ESI) m/z: 582 (M+H)⁺.

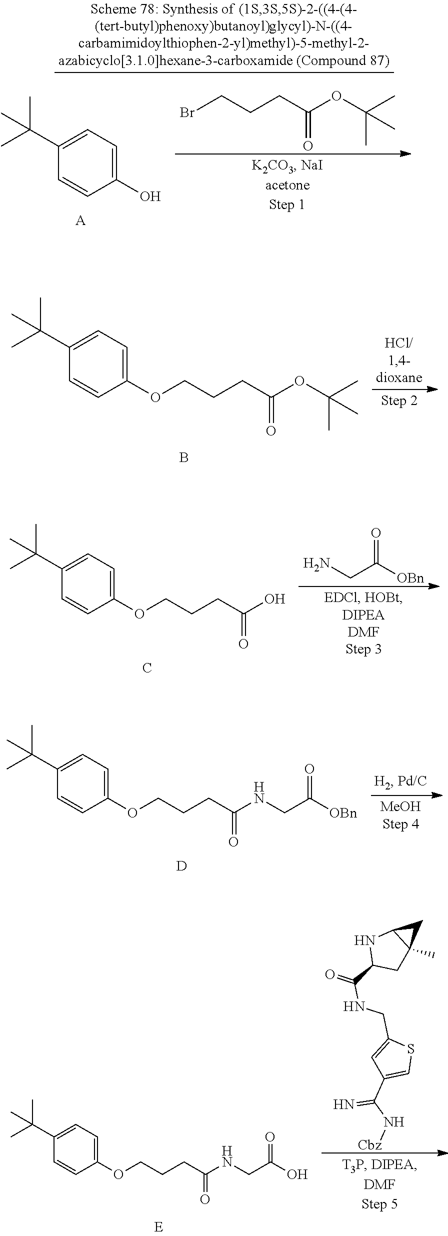

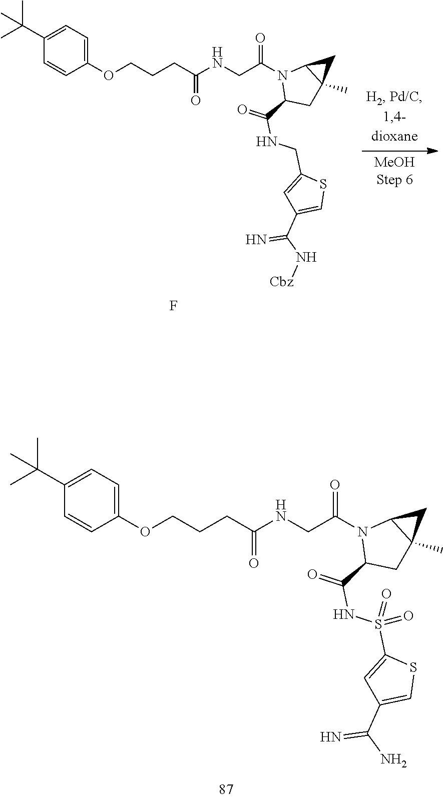

Step 1: tert-butyl 4-(4-(tert-butyl)phenoxy)butanoate (B). To a mixture of compound A (1 g, 6.66 mmol) and tert-butyl 4-bromobutanoate (1.5 g, 6.66 mmol) in acetone (30 mL) was added K₂CO₃ (2.8 g, 19.97 mmol) and NaI (250 mg, 1.66 mmol) at room temperature and the mixture was stirred under N₂ atmosphere at 60° C. for 16 hours. The mixture was diluted with saturated aq. NH₄Cl solution and extracted with EtOAc twice. The combined organic layers were washed with brine, dried over anhydrous Na₂SO₄, filtered and concentrated to dryness under reduced pressure. The residue was purified by flash chromatography on silica gel (PE:EtOAc=1:1) to give compound B (580 mg, yield 29.8%) as a white solid.

Step 2: 4-(4-(tert-butyl)phenoxy)butanoic acid (C). To a solution of compound B (580 mg, 1.98 mmol) in 1,4-dioxane (5 mL) was added HCl/1,4-dioxane (5 mL) at 0° C. and the reaction was stirred at room temperature for 2 hours. The mixture was concentrated to dryness under reduced pressure, co-evaporated with DCM twice and dried under vacuum to give compound C (450 mg, yield 96.0%) as a white solid. LC/MS (ESI) m/z: 235 (M−H)⁻.

Step 3: benzyl (4-(4-(tert-butyl)phenoxy)butanoyl)glycinate (D). To a mixture of compound C (450 mg, 1.90 mmol) and benzyl 2-aminoacetate (0.5 g, 2.86 mmol) in DMF (10 ml) was added DIPEA (1.2 g, 9.52 mmol), EDCI (0.7 g, 3.81 mmol), and HOBt (0.4 g, 2.86 mmol) at 0° C. and the mixture was stirred under N₂ atmosphere at room temperature for 16 hours. The mixture was diluted with saturated aq.NH₄Cl solution and extracted with EtOAc twice. The combined organic layers were washed with brine, dried over anhydrous Na₂SO₄, filtered and concentrated to dryness under reduced pressure. The residue was purified by flash chromatography on silica gel (PE:EtOAc=1:1) to give compound D (600 mg, yield 82.2%) as a white solid. LC/MS (ESI) m/z: 384 (M+H)⁺.

Step 4: (4-(4-(tert-butyl)phenoxy)butanoyl)glycine (E). To a solution of compound D (600 mg, 1.57 mmol) in MeOH (10 mL) was added Pd/C (100 mg, 10% wt) at room temperature under N₂ atmosphere and the reaction was stirred under a H₂ balloon at room temperature for 16 hours. The mixture was filtered and the filtrate was concentrated to dryness under reduced pressure to give compound E (450 mg, yield 98.0%) as a white solid. LC/MS (ESI) m/z: 294 (M+H)⁺.

Step 5: benzyl ((5-(((1S,3S,5S)-2-((4-(4-(tert-butyl)phenoxy)butanoyl)glycyl)-5-methyl-2-azabicyclo[3.1.0]hexane-3-carboxamido)methyl)thiophen-3-yl)(imino)methyl)carbamate (F). To a mixture of compound E (36 mg, 0.12 mmol) and benzyl (imino(5-(((1S,3S,5S)-5-methyl-2-azabicyclo[3.1.0]hexane-3-carboxamido)methyl)thiophen-3-yl)methyl)carbamate (100 mg, 0.12 mmol) in DMF (5 mL) was added DIPEA (94 mg, 0.73 mmol) and T₃P (231 mg, 0.36 mmol, 50% wt in EtOAc) at 0° C. and the mixture was stirred under N₂ atmosphere at room temperature for 3 hours. The mixture was diluted with EtOAc and washed with saturated aq. NaHCO₃ solution. The aqueous layer was extracted with EtOAc twice. The combined organic layers were washed with brine, dried over anhydrous Na₂SO₄, filtered and concentrated to dryness under reduced pressure. The residue was purified by flash chromatography on silica gel (DCM:MeOH=20:1) to give compound F (80 mg, yield 96.0%) as a white solid. LC/MS (ESI) m/z: 688 (M+H)⁺.

Step 6: (1S,3S,5S)-2-((4-(4-(tert-butyl)phenoxy)butanoyl)glycyl)-N-((4-carbamimidoyl-thiophen-2-yl)methyl)-5-methyl-2-azabicyclo[3.1.0]hexane-3-carboxamide (Compound 87). To a solution of compound F (80 mg, 0.12 mmol) in MeOH (5 mL) was added Pd/C (100 mg, 10% wt) and HCl/1,4-dioxane (0.1 mL) at 0° C. under N₂ atmosphere and the reaction was stirred under a H₂ balloon at room temperature for 16 hours. The mixture was filtered and the filtrate was concentrated to dryness under reduced pressure. The residue was purified by prep-HPLC to give Compound 87 (16 mg, yield 24.8%) as a white solid. ¹H NMR (400 MHz, CD₃OD) δ 8.22 (s, 1H), 7.40 (s, 1H), 7.29-7.26 (m, 2H), 6.83-6.81 (m, 2H), 4.82-4.78 (m, 1H), 4.57-4.46 (m, 2H), 4.16 (d, J=12.0 Hz, 2H), 3.98 (t, J=6.3 Hz, 2H), 3.35-3.33 (m, 1H), 2.48-2.44 (m, 2H), 2.41-2.35 (m, 1H), 2.16 (dd, J=13.3, 3.2 Hz, 1H), 2.09-2.02 (m, 2H), 1.28 (s, 12H), 1.12 (dd, J=5.5, 2.3 Hz, 1H), 0.80 (t, J=6.4 Hz, 1H); LC/MS (ESI) m/z: 554 (M+H)⁺.

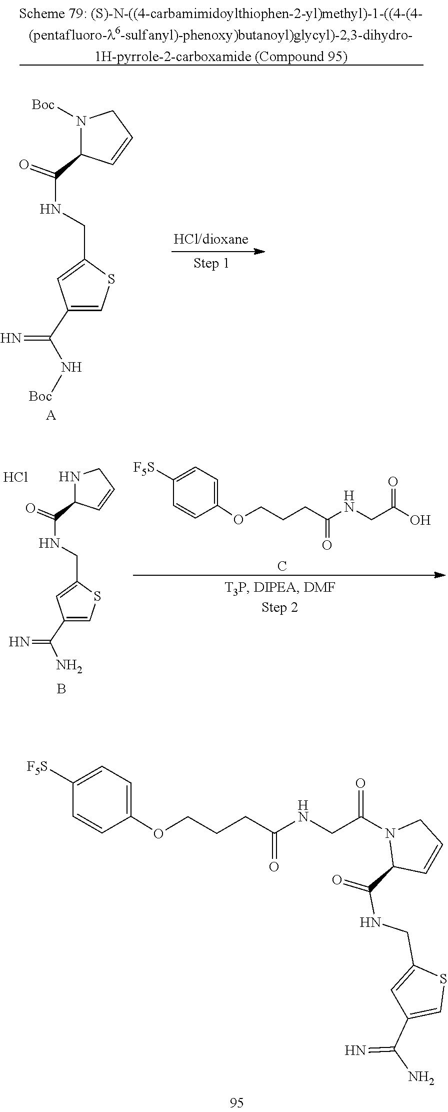

Step 1: (S)-N-((4-carbamimidoylthiophen-2-yl)methyl)-2,3-dihydro-1H-pyrrole-2-carboxamide hydrochloride (B). A solution of tert-butyl (S)-2-(((4-(N-(tert-butoxycarbonyl)-carbamimidoyl)thiophen-2-yl)methyl)carbamoyl)-2,3-dihydro-1H-pyrrole-1-carboxylate (A; 90 mg, 0.20 mmol) in HCl/1,4-dioxane (1 mL) was stirred at 25° C. for 1 hour. The mixture was concentrated to dryness under reduced pressure to give (S)-N-((4-carbamimidoylthiophen-2-yl)methyl)-2,3-dihydro-1H-pyrrole-2-carboxamide hydrochloride (B; 44 mg, 88.0% yield) as a white solid, which was used directly in the next step without further purification. LC/MS (ESI) m/z: 251 (M+H)⁺.

Step 2: (S)-N-((4-carbamimidoylthiophen-2-yl)methyl)-1-((4-(4-(pentafluoro-λ⁶-sulfanyl)-phenoxy)butanoyl)glycyl)-2,3-dihydro-1H-pyrrole-2-carboxamide (Compound 96). To a mixture of (4-(4-(pentafluoro-λ⁶-sulfanyl)phenoxy)butanoyl)glycine (C; 42 mg, 0.12 mmol) and (S)-N-((4-carbamimidoylthiophen-2-yl)methyl)-2,3-dihydro-1H-pyrrole-2-carboxamide hydrochloride (B; 44 mg, 0.18 mmol) in DMF (1 mL) was added DIPEA (47 mg, 0.36 mmol) and T₃P (114 mg, 0.18 mmol, 50% in ethyl acetate) at room temperature under N₂ atmosphere and the mixture was stirred at room temperature overnight. The mixture was quenched with saturated aq. NaHCO₃ solution and extracted with ethyl acetate twice. The combined organic layers were washed with water and brine, dried over anhydrous Na₂SO₄, filtered and concentrated to dryness under reduced pressure. The residue was purified by prep-HPLC to give (S)-N-((4-carbamimidoylthiophen-2-yl)methyl)-1-((4-(4-(pentafluoro-λ⁶-sulfanyl)phenoxy)butanoyl) glycyl)-2,3-dihydro-1H-pyrrole-2-carboxamide (Compound 95; 1.1 mg, 1.5% yield) as white solid. ¹H NMR (400 MHz, CD₃OD) δ 8.21 (d, J=1.6 Hz, 1H), 7.71 (d, J=9.2 Hz, 2H), 7.40 (s, 1H), 7.03 (d, J=8.9 Hz, 2H), 6.08 (dd, J=6.4, 2.1 Hz, 1H), 5.85 (dd, J=6.3, 2.2 Hz, 1H), 5.12 (s, 1H), 4.54 (s, 2H), 4.45 (s, 2H), 4.12-4.03 (m, 4H), 2.47 (t, J=7.4 Hz, 2H), 2.10 (t, J=6.9 Hz, 2H); LC/MS (ESI) m/z: 596 (M+H)⁺.

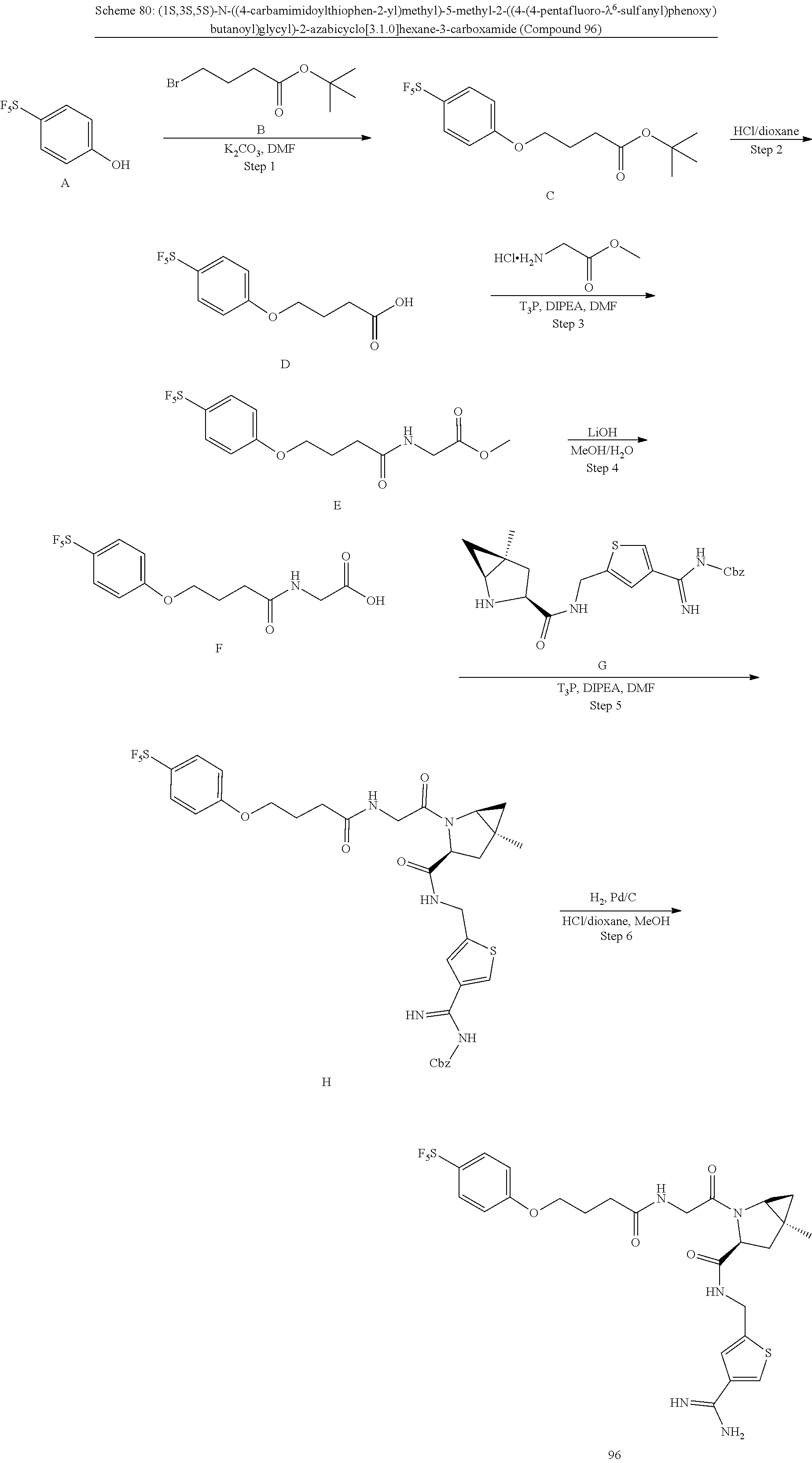

Step 1: tert-butyl 4-(4-(pentafluoro-λ⁶-sulfanyl)phenoxy)butanoate (C). To a mixture of 4-(pentafluoro-λ⁶-sulfanyl)phenol (A; 500 mg, 2.27 mmol) and tert-butyl 4-bromobutanoate (B; 506 mg, 2.27 mmol) in DMF (5 mL) was added K₂CO₃ (941 mg, 6.81 mmol) and NaI (340 mg, 2.27 mmol) and the reaction was stirred at 120° C. for 16 hours. The mixture was diluted with ethyl acetate and washed with saturated aq.NH₄Cl solution. The aqueous layer was extracted with ethyl acetate twice and the combined organic layers were washed with brine, dried over anhydrous Na₂SO₄, filtered and concentrated to dryness under reduced pressure. The residue was purified by flash chromatography on silica gel (petroleum ether:ethyl acetate=3:1 to 1:1) to give tert-butyl 4-(4-(pentafluoro-λ⁶-sulfanyl)-phenoxy)butanoate (C; 550 mg, yield 66.7%) as a white solid. LC/MS (ESI) m/z: 363 (M−H)⁻.

Step 2: 4-(4-(pentafluoro-16-sulfanyl)phenoxy)butanoic acid (D). To a solution of tert-butyl 4-(4-(pentafluoro-λ⁶-sulfanyl)phenoxy)butanoate (C; 550 mg, 1.52 mmol) in dichloromethane (5 mL) was added HCl/1,4-dioxane (5 mL) at 0° C. and the reaction was stirred at room temperature for 2 hours. The mixture was concentrated to dryness under reduced pressure, co-evaporated with dichloromethane twice and dried under reduced pressure to give 4-(4-(pentafluoro-λ⁶-sulfanyl)phenoxy)butanoic acid (D; 500 mg, yield 96.2%) as a white solid, which was used directly in the next step without further purification. LC/MS (ESI) m/z: 305 (M−H)⁻.

Step 3: methyl (4-(4-(pentafluoro-λ⁶-sulfanyl)phenoxy)butanoyl)glycinate (E). To a mixture of 4-(4-(pentafluoro-λ⁶-sulfanyl)phenoxy)butanoic acid (D; 500 mg, 1.63 mmol) and methyl glycinate hydrochloride (308 mg, 2.45 mmol) in DMF (6 ml) was added DIPEA (632 mg, 4.89 mmol), and T₃P (1.56 g, 4.89 mmol, 50% in ethyl acetate). The mixture was stirred at room temperature under N₂ atmosphere overnight. The reaction was quenched with saturated aq.NaHCO₃ solution. The resulting mixture was extracted with ethyl acetate (3×20 mL). The combined organic layers were washed with water (2×20 mL) and brine (2×20 mL), dried over anhydrous Na₂SO₄, filtered and concentrated to dryness under reduced pressure. The residue was purified by column chromatography on silica gel (ethyl acetate:petroleum ether=1:5 to 1:1) to give methyl (4-(4-(pentafluoro-λ⁶-sulfanyl)phenoxy)-butanoyl)glycinate (E; 370 mg, 61.4% yield) as a yellow oil. LC/MS (ESI) m/z: 371 (M+H)⁺.

Step 4: (4-(4-(pentafluoro-λ⁶-sulfanyl)phenoxy)butanoyl)glycine (F). To a solution of methyl (4-(4-(pentafluoro-λ⁶-sulfanyl)phenoxy)butanoyl)glycinate (E; 370 mg, 0.98 mmol) in methanol (3 mL) and water (1 mL) was added a solution of LiOH·H₂O (124 mg, 2.94 mmol) at 0° C. and the mixture was stirred at room temperature for 2 hours. The mixture was acidified with 1 N aq. HCl solution to pH<3 and extracted with dichloromethane (2×10 mL). The combined organic layers were concentrated to dryness under reduced pressure to give (4-(4-(pentafluoro-λ⁶-sulfanyl)phenoxy)butanoyl)glycine (F; 240 mg, 67.6% yield) as a white solid, which was used directly in next step without further purification. LC/MS (ESI) (m/z): 364(M+H)⁺.

Step 5: benzyl (imino(5-(((1S,3S,5S)-5-methyl-2-((4-(4-(pentafluoro-λ⁶-sulfanyl)phenoxy) butanoyl)glycyl)-2-azabicyclo[3.1.0]hexane-3-carboxamido)methyl)thiophen-3-yl)methyl) carbamate (H). To a mixture of (4-(4-(pentafluoro-λ⁶-sulfanyl)phenoxy)butanoyl)glycine (F; 30 mg, 0.081 mmol) and benzyl (imino(5-(((1 S,3S,5S)-5-methyl-2-azabicyclo[3.1.0]hexane-3-carboxamido)methyl) thiophen-3-yl)methyl)carbamate (G; 40 mg, 0.10 mmol) in DMF (1.0 mL) was added DIPEA (31 mg, 0.24 mmol) and T₃P (78 mg, 0.12 mmol, 50% in ethyl acetate) at 0° C. and the mixture was stirred under N₂ atmosphere at 25° C. for 16 hours. The reaction was quenched with saturated aq. NaHCO₃ solution. The resulting mixture was extracted with ethyl acetate (3×20 mL). The combined organic layers were washed with water (2×20 mL) and brine (2×20 mL), dried over anhydrous Na₂SO₄, filtered and concentrated to dryness under reduced pressure. The residue was purified by flash chromatography on silica gel (dichloromethane:methanol=20:1 to 12:1) to give benzyl (imino(5-(((1 S,3S,5S)-5-methyl-2-((4-(4-(pentafluoro-λ⁶-sulfanyl)phenoxy)butanoyl)glycyl)-2-azabicyclo[3.1.0]hexane-3-carboxamido)-methyl)-thiophen-3-yl)methyl)carbamate (H; 20 mg, yield 32.8%) as a yellow oil. LC/MS (ESI) (m/z): 758 (M+H)⁺.

Step 6: (1S,3S,5S)-N-((4-carbamimidoylthiophen-2-yl)methyl)-5-methyl-2-((4-(4-(pentafluoro-λ⁶-sulfanyl)phenoxy)butanoyl)glycyl)-2-azabicyclo[3.1.0]hexane-3-carboxamide (Compound 96). To a solution of benzyl (imino(5-(((1 S,3S,5S)-5-methyl-2-((4-(4-(pentafluoro-λ⁶-sulfanyl)phenoxy)-butanoyl)glycyl)-2-azabicyclo[3.1.0]hexane-3-carboxamido)methyl)thiophen-3-yl) methyl)carbamate (H; 20 mg, 0.03 mmol) in methanol (1.0 mL) was added Pd/C (2 mg, 10% wt) and HCl/1,4-dioxane (1 drop) at 25° C. and the mixture was degassed under H₂ atmosphere for three times and stirred under a H₂ balloon at 25° C. for 16 hours. The mixture was filtered and filtrate was concentrated to dryness under reduced pressure. The residue was purified by prep-HPLC to give (1S,3S,5S)-N-((4-carbamimidoylthiophen-2-yl)methyl)-5-methyl-2-((4-(4-(pentafluoro-λ⁶-sulfanyl)phenoxy)butanoyl)glycyl)-2-azabicyclo[3.1.0]hexane-3-carboxamide (Compound 96; 4.5 mg, yield 28.1%) as white solid. ¹H NMR (400 MHz, CD₃OD) δ 8.53 (s, 1H), 8.21 (d, J=1.6 Hz, 1H), 7.73-7.69 (m, 2H), 7.39 (s, 1H), 7.02 (d, J=9.0 Hz, 2H), 4.81-4.77 (m, 1H), 4.58 (s, 1H), 4.51 (q, J=15.8 Hz, 2H), 4.21 (d, J=16.7 Hz, 1H), 4.11 (dd, J=15.1, 8.8 Hz, 3H), 2.47 (t, J=7.3 Hz, 2H), 2.38 (t, J=12.3 Hz, 1H), 2.17-2.08 (m, 3H), 1.28 (s, 3H), 1.13 (dd, J=5.8, 2.5 Hz, 1H), 0.80 (t, J=5.5 Hz, 1H); LC/MS (ESI) (m/z): 624 (M+H)⁺.

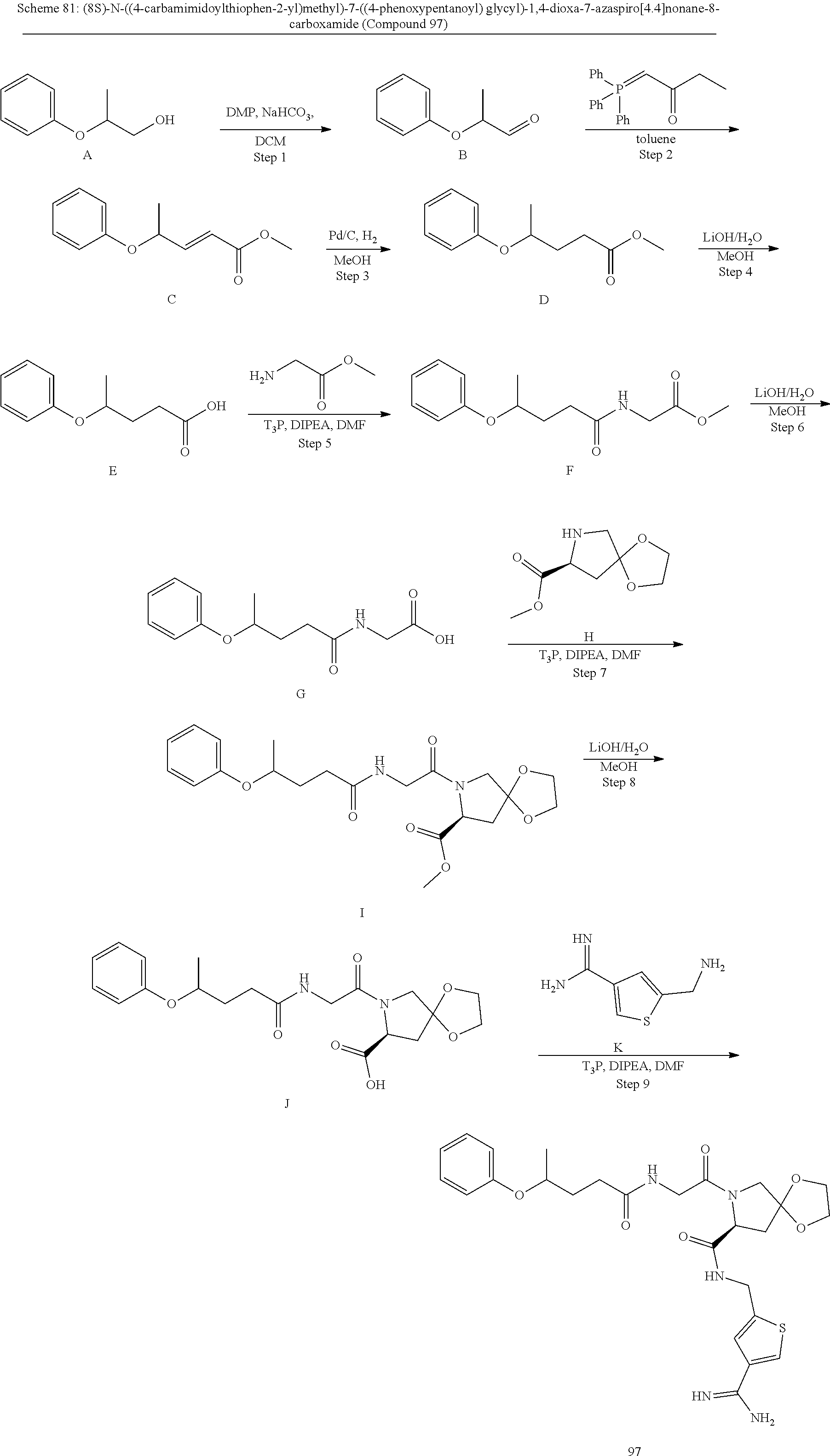

Step 1: 2-phenoxypropanal (B). To a solution of 2-phenoxypropan-1-ol (A; 3 g, 19.71 mmol) in dichloromethane (30 mL) was added K₂CO₃ (5 g, 59.13 mmol) and DMP (17 g, 39.42 mmol) and the reaction was stirred at room temperature for 2 hours. The mixture was diluted with water and extracted with dichloromethane twice. The layers were separated and the organic layer was washed with brine, dried over anhydrous Na₂SO₄ and concentrated to dryness under reduced pressure. The residue was purified by flash chromatography on silica gel (petroleum ether:ethyl acetate=20:1 to 1:1) to give 2-phenoxypropanal (B; 2.0 g, yield 67.8%) as a white solid. LC/MS (ESI) m/z: 151 (M+H)⁻.

Step 2: methyl (E)-4-phenoxypent-2-enoate (C). At room temperature, a suspension of 2-phenoxypropanal (B; 1 g, 6.7 mmol), and methyl 2-(triphenyl-15-phosphanylidene)acetate (2.2 g, 6.7 mmol) in toluene (10 mL) was heated up to 110° C. and stirred under N₂ atmosphere for 18 hours. The mixture was diluted with water and extracted with dichloromethane twice. The combined organic layers were washed with brine, dried over anhydrous Na₂SO₄ and concentrated to dryness under reduced pressure. The residue was purified by flash chromatography on silica gel (dichloromethane:methanol=50:1 to 20:1) to give methyl (E)-4-phenoxypent-2-enoate (C; 550 mg, Yield 40%). LC/MS (ESI) m/z: 207 (M+H)⁺.

Step 3: methyl 4-phenoxypentanoate (D). To a solution of methyl (E)-4-phenoxypent-2-enoate (C; 550 mg, 2.7 mmol) in methanol (5.0 mL) was added Pd/C (55 mg, 10% wt) at 25° C. under N₂ atmosphere and the mixture was stirred under a H₂ balloon at 25° C. for 16 hours. The mixture was filtered and filtrate was concentrated to dryness under reduced pressure. The residue was purified by flash chromatography on silica gel (dichloromethane:methanol=50:1 to 20:1) to give methyl 4-phenoxypentanoate (D; 300 mg, Yield 53.2%). LC/MS (ESI) (m/z): 209 (M+H)⁺.

Step 4: 4-phenoxypentanoic acid (E). To a solution of methyl 4-phenoxypentanoate (D; 300 mg, 1.44 mmol) in methanol (3 mL) and water (1 mL) was added a solution of LiOH·H₂O (181 mg, 4.32 mmol) at 0° C. and the mixture was stirred at room temperature for 2 hours. The mixture was acidified with 1 N aq. HCl solution to pH<3 and extracted with dichloromethane (2×10 mL). The combined organic layers were concentrated to dryness under reduced pressure to give 4-phenoxypentanoic acid (280 mg, 98.2% yield) as a white solid, which was used directly in next step without further purification. LC/MS (ESI) (m/z): 195(M+H)⁺.

Step 5: methyl (4-phenoxypentanoyl)glycinate (F). To a mixture of 4-phenoxypentanoic acid (E; 280 mg, 1.44 mmol) and methyl glycinate hydrochloride (271 mg, 2.16 mmol) in DMF (3.0 mL) was added DIPEA (558 mg, 4.32 mmol) and T₃P (1.37 g, 2.16 mmol, 50% in ethyl acetate) at 0° C. and the mixture was stirred under N₂ atmosphere at 25° C. for 16 hours. The reaction was quenched with saturated aq.NaHCO₃ solution. The resulting mixture was extracted with ethyl acetate (3×20 mL). The combined organic layers were washed with water (2×20 mL) and brine (2×20 mL), dried over anhydrous Na₂SO₄, filtered and concentrated to dryness under reduced pressure. The residue was purified by flash chromatography on silica gel (dichloromethane:methanol=20:1 to 12:1) to give methyl (4-phenoxypentanoyl)glycinate (F; 220 mg, yield 57.4%) as a yellow oil. LC/MS (ESI) (m/z): 266 (M+H)⁺.

Step 6: (4-phenoxypentanoyl)glycine (G). To a solution of (4-phenoxypentanoyl)glycinate (F; 220 mg, 0.83 mmol) in methanol (3 mL) and water (1 mL) was added a solution of LiOH·H₂O (104 mg, 2.49 mmol) at 0° C. and the mixture was stirred at room temperature for 2 hours. The mixture was acidified with 1 N aq. HCl solution to pH<3 and extracted with dichloromethane (2×10 mL). The combined organic layers were concentrated to dryness under reduced pressure to give (4-phenoxy-pentanoyl)glycine (G; 110 mg, 52.6% yield) as a white solid, which was used directly in next step without further purification. LC/MS (ESI) (m/z): 252(M+H)⁺.

Step 7: methyl (8S)-7-((4-phenoxypentanoyl)glycyl)-1,4-dioxa-7-azaspiro[4.4]nonane-8-carboxylate (1). To a mixture of (4-phenoxypentanoyl)glycine (G; 100 mg, 0.40 mmol) and methyl (S)-1,4-dioxa-7-azaspiro[4.4]nonane-8-carboxylate (H; 112 mg, 0.60 mmol) in DMF (2.0 mL) was added DIPEA (154 mg, 1.19 mmol) and T₃P (380 mg, 0.60 mmol, 50% in ethyl acetate) at 0° C. and the mixture was stirred under N₂ atmosphere at 25° C. for 16 hours. The reaction was quenched with saturated aq.NaHCO₃ solution. The resulting mixture was extracted with ethyl acetate (3×20 mL). The combined organic layers were washed with water (2×20 mL) and brine (2×20 mL), dried over anhydrous Na₂SO₄, filtered and concentrated to dryness under reduced pressure. The residue was purified by flash chromatography on silica gel (dichloromethane:methanol=20:1 to 12:1) to give methyl (8S)-7-((4-phenoxypentanoyl)glycyl)-1,4-dioxa-7-azaspiro[4.4]nonane-8-carboxylate (60 mg, yield 35.7%) as a yellow oil. LC/MS (ESI) (m/z): 421 (M+H)⁺.

Step 8: (8S)-7-((4-phenoxypentanoyl)glycyl)-1,4-dioxa-7-azaspiro[4.4]nonane-8-carboxylic acid (J). To a solution of methyl (8S)-7-((4-phenoxypentanoyl)glycyl)-1,4-dioxa-7-azaspiro[4.4]nonane-8-carboxylate (I; 60 mg, 0.14 mmol) in methanol (0.75 mL) and water (0.25 mL) was added a solution of LiOH·H₂O (20 mg, 0.43 mmol) at 0° C. and the mixture was stirred at room temperature for 2 hours. The mixture was acidified with 1 N aq. HCl solution to pH<3 and extracted with dichloromethane (2×5 mL). The combined organic layers were concentrated to dryness under reduced pressure to give (8S)-7-((4-phenoxypentanoyl)glycyl)-1,4-dioxa-7-azaspiro[4.4]nonane-8-carboxylic acid (J; 50 mg, 86.2% yield) as a white solid, which was used directly in next step without further purification. LC/MS (ESI) (m/z): 407(M+H)⁺.

Step 9: (8S)-N-((4-carbamimidoylthiophen-2-yl)methyl)-7-((4-phenoxypentanoyl)glycyl)-1,4-dioxa-7-azaspiro[4.4]nonane-8-carboxamide (Compound 98). To a solution of (8S)-7-((4-phenoxy-pentanoyl)glycyl)-1,4-dioxa-7-azaspiro[4.4]nonane-8-carboxylic acid (J; 50 mg, 0.12 mmol) and 5-(aminomethyl)thiophene-3-carboximidamide (K; 35 mg, 0.19 mmol) in DMF (1 mL) was added DIPEA (48 mg, 0.37 mmol) and T₃P (118 mg, 0.19 mmol, 50% in ethyl acetate). The mixture was stirred at room temperature under N₂ atmosphere overnight. The reaction was quenched with saturated aq.NaHCO₃ solution. The resulting mixture was extracted with ethyl acetate (3×5 mL). The combined organic layers were washed with water (2×5 mL) and brine (2×5 mL), dried over anhydrous Na₂SO₄, filtered and concentrated to dryness under reduced pressure. The residue was purified by prep-HPLC to give (8S)-N-((4-carbamimidoylthiophen-2-yl)methyl)-7-((4-phenoxypentanoyl)glycyl)-1,4-dioxa-7-azaspiro[4.4]nonane-8-carboxamide (3.3 mg, 5.0% yield) as a white solid. ¹H NMR (400 MHz, CD₃OD) δ 8.53 (s, 1H), 8.22 (dd, J=5.1, 3.4 Hz, 1H), 7.48-7.39 (m, 1H), 7.25-7.20 (m, 2H), 6.91-6.86 (m, 3H), 4.61 (s, 1H), 4.58-4.50 (m, 3H), 4.46 (d, J=6.1 Hz, 1H), 3.99-3.93 (m, 5H), 3.69 (s, 2H), 2.48-2.36 (m, 3H), 2.20 (d, J=5.4 Hz, 1H), 2.02-1.89 (m, 2H), 1.27 (dt, J=6.1, 2.0 Hz, 3H); LC/MS (ESI) (m/z): 544(M+H)⁺.

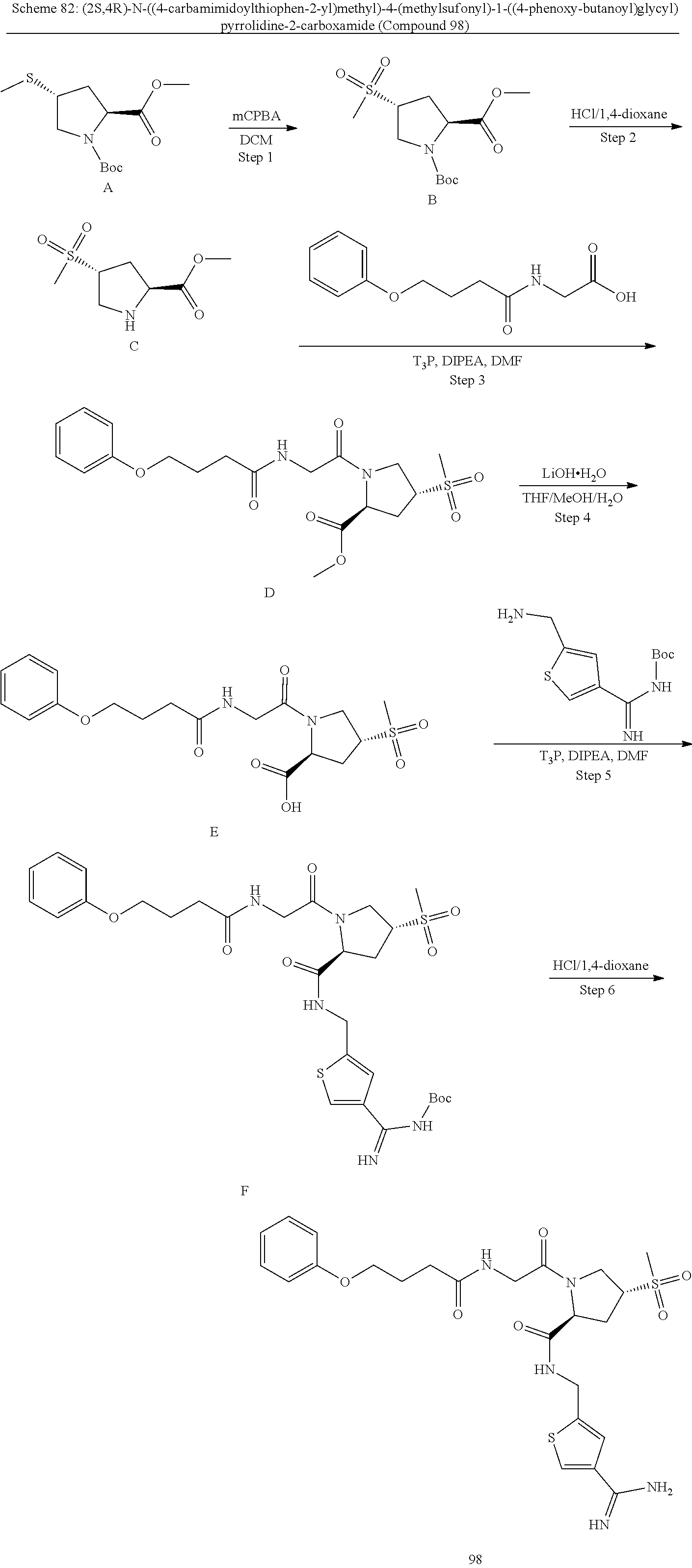

Step 1: 1-(tert-butyl) 2-methyl (2S,4R)-4-(methylsulfonyl)pyrrolidine-1,2-dicarboxylate (B). To a solution of compound B (400 mg, 1.45 mmol) in DCM (4 mL) was added mCPBA (883 mg, 4.36 mmol, 85% purity) at 0° C. and the mixture was stirred at 25° C. for 16 hours. The mixture was diluted with DCM and washed with saturated aq.NaHCO₃ solution. The aqueous layer was extracted with DCM twice. The combined organic layers were washed with brine, dried over anhydrous Na₂SO₄, filtered and concentrated to dryness under reduced pressure. The residue was purified by flash chromatography on silica gel (PE:EtOAc=1:1) to give compound B (272 mg, yield 61.1%) as a light-yellow oil. LC/MS (ESI) m/z: 208 (M−100+−H)⁺.

Step 2: methyl (2S,4R)-4-(methylsulfonyl)pyrrolidine-2-carboxylate (C). A mixture of compound B (272 mg, 0.89 mmol) in HCl/1,4-dioxane (3 mL, 4 M) was stirred at room temperature for 4 hours. The reaction mixture was concentrated to dryness under reduced pressure, co-evaporated with DCM twice and dried under vacuum to give compound C (183 mg, yield 99.7%) as a white solid, which was used directly in the next step. LC/MS (ESI) m/z: 208 (M+H)⁺.

Step 3: methyl (2S,4R)-4-(methylsulfonyl)-1-((4-phenoxybutanoyl)glycyl)pyrrolidine-2-carboxylate (D). To a mixture of compound C (50 mg, 0.24 mmol) and (4-phenoxybutanoyl)glycine (63 mg, 0.26 mmol) in DMF (1 mL) was added DIPEA (0.24 mL, 1.44 mmol) and T₃P (458 mg, 0.72 mmol, 50% wt in EtOAc) at 0° C. and the mixture was stirred under N₂ atmosphere at 35° C. for 4 hours. The mixture was diluted with saturated aq. NaHCO₃ solution and extracted with CHCl₃/i-PrOH (v/v=3:1) five times. The combined organic layers were washed with brine, dried over anhydrous Na₂SO₄, filtered and concentrated to dryness under reduced pressure. The residue was purified by flash chromatography on silica gel (DCM:MeOH=20:1) to give compound D (89 mg, yield 83.3%) as a yellow solid. LC/MS (ESI) m/z: 427 (M+H)⁺.

Step 4: (2S,4R)-4-(methylsulfonyl)-1-((4-phenoxybutanoyl)glycyl)pyrrolidine-2-carboxylic acid (E). To a solution of compound D (89 mg, 0.21 mmol) in MeOH (1 mL), THE (0.5 mL) and H₂O (0.5 mL) was added LiOH·H₂O (9 mg, 0.21 mmol) at 0° C. and the mixture was stirred at 25° C. for 4 hours. The mixture was diluted with water and washed with EtOAc. The aqueous layer was acidified with 0.5 M aq. HCl solution and extracted with EtOAc twice. The combined organic layers were washed with brine, dried over anhydrous Na₂SO₄, filtered and concentrated to dryness under reduced pressure to give compound E (63 mg, yield 73.3%) as a light-yellow oil, which was used directly in the next step. LC/MS (ESI) m/z: 413 (M+H)⁺.

Step 5: (2S,4R)-4-(methylsulfonyl)-1-((4-phenoxybutanoyl)glycyl)pyrrolidine-2-carboxylic acid (F). To a mixture of compound E (63 mg, 0.15 mmol) and tert-butyl ((5-(aminomethyl)thiophen-3-yl)(imino)methyl)carbamate (39 mg, 0.15 mmol) in DMF (2 mL) was added DIPEA (0.15 mL, 0.9 mmol) and T₃P (286 mg, 0.45 mmol, 50% wt in EtOAc) at 0° C. and the mixture was stirred under N₂ atmosphere at 35° C. for 4 hours. The mixture was diluted with saturated aq.NaHCO₃ solution and extracted with CHCl₃/i-PrOH (v/v=3:1) five times. The combined organic layers were washed with brine, dried over anhydrous Na₂SO₄, filtered and concentrated to dryness under reduced pressure. The residue was purified by flash chromatography on silica gel (DCM:MeOH=10:1) to give compound F (30 mg, yield 30.9%) as a yellow solid. LC/MS (ESI) m/z: 650 (M+H)⁺.

Step 6: (2S,4R)-N-((4-carbamimidoylthiophen-2-yl)methyl)-4-(methylsulfonyl)-1-((4-phenoxybutanoyl)glycyl)pyrrolidine-2-carboxamide (Compound 98). A mixture of compound F (30 mg, 0.05 mmol) in HCl/1,4-dioxane (2 mL) was stirred at 30° C. for 8 hours. The reaction mixture was concentrated to dryness under reduced pressure, co-evaporated with DCM twice and dried under vacuum. The residue was purified by prep-HPLC to give Compound 98 (3.3 mg, yield 13.8%) as a white solid. ¹H NMR (400 MHz, CD₃OD) δ 8.22 (d, J=5.2 Hz, 1H), 7.41 (s, 1H), 7.26-7.20 (m, 2H), 6.91-6.85 (m, 3H), 4.70-4.64 (m, 1H), 4.58 (s, 2H), 4.13-4.05 (m, 4H), 3.99 (t, J=5.8 Hz, 2H), 3.97-3.81 (m, 1H), 3.05 (s, 3H), 2.74-2.68 (m, 1H), 2.48-2.44 (m, 2H), 2.42-2.34 (m, 1H), 2.10-2.03 (m, 2H); LC/MS (ESI) m/z: 550 (M+H)⁺.

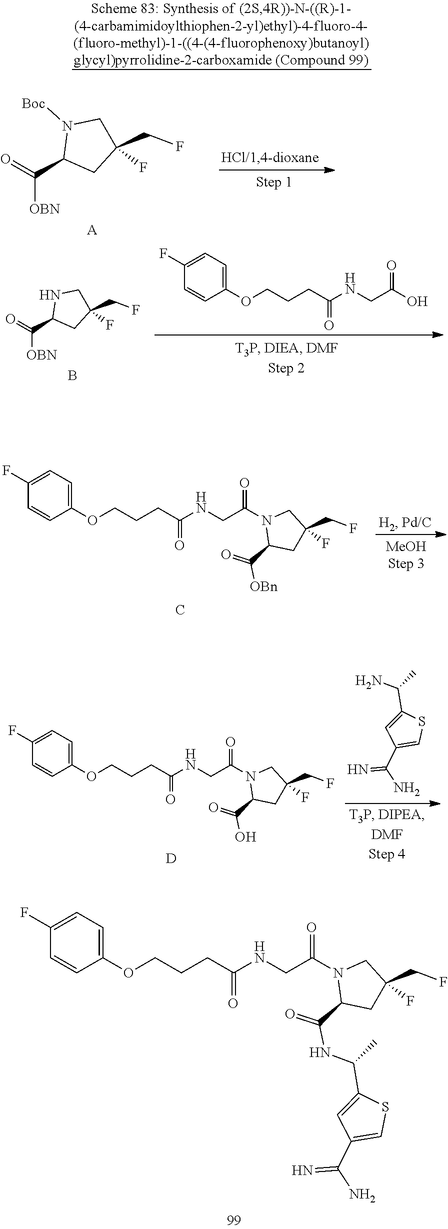

Step 1: benzyl (2S,4R)-4-fluoro-4-(fluoromethyl)pyrrolidine-2-carboxylate (B). A mixture of compound 2 (190 mg, 0.54 mmol) in HCl/1,4-dioxane (3 mL, 4 M) was stirred at 25° C. for 2 hours. The reaction mixture was concentrated to dryness under reduced pressure, co-evaporated with DCM twice and dried under vacuum to give compound B (136 mg, 98.6% yield) as yellow oil, which was used directly in the next step. LC/MS (ESI) m/z: 256 (M+H)⁺.

Step 2: benzyl (2S,4R)-4-fluoro-4-(fluoromethyl)-1-{2-[4-(4-fluorophenoxy)butanamido]-acetyl}pyrrolidine-2-carboxylate (C). To a mixture of compound B (136 mg, 0.53 mmol) and [4-(4-fluorophenoxy)butanamido]acetic acid (150 mg, 0.59 mmol) in DMF (4 mL) was added DIPEA (0.56 mL, 3.2 mmol) and T₃P (509 mg, 1.60 mmol, 50% wt in EtOAc) at 0° C. and the mixture was stirred under N₂ atmosphere at 25° C. for 2 hours. The mixture was diluted with EtOAc and washed with saturated aq.NaHCO₃ solution. The aqueous layer was extracted with EtOAc twice. The combined organic layers were washed with brine, dried over anhydrous Na₂SO₄, filtered and concentrated to dryness under reduced pressure. The residue was purified by flash chromatography on silica gel (PE:EtOAc=1:1) to give compound C (195 mg, 74.3% yield) as a light-yellow oil. LC/MS (ESI) (m/z): 493 (M+H)⁺.

Step 3: (2S,4R)-4-fluoro-4-(fluoromethyl)-1-{2-[4-(4-fluorophenoxy)butanamido]-acetyl}-pyrrolidine-2-carboxylic acid (D). To a solution of compound C (190 mg, 0.39 mmol) in MeOH (4 mL) was added Pd/C (60 mg, 10% wt) at 0° C. under N₂ atmosphere and the mixture was stirred under a H₂ balloon at room temperature for 2.5 hours. The mixture was filtered and the filtrate was concentrated to dryness under reduced pressure to give compound D (140 mg, 90.4% yield) as a colorless oil, which was used directly in the next step. LC/MS (ESI) (m/z): 403 (M+H)⁺.

Step 4: (2S,4R)-N-[(1R)-1-(4-carbamimidoylthiophen-2-yl)ethyl]-4-fluoro-4-(fluoromethyl)-1-{2-[4-(4-fluorophenoxy)butanamido]acetyl}pyrrolidine-2-carboxamide (Compound 99). To a mixture of compound D (146 mg, 0.36 mmol) and 5-[(1R)-1-aminoethyl]thiophene-3-carboximidamide (92 mg, 0.54 mmol) in DMF (4 mL) was added DIPEA (0.38 mL, 2.18 mmol) and T₃P (693 mg, 1.09 mmol, 50% wt in EtOAc) at 0° C. and the mixture was stirred under N₂ atmosphere at 30° C. for 18 hours. The mixture was diluted with EtOAc and washed with saturated aq. NaHCO₃ solution. The aqueous layer was extracted with CHCl₃/i-PrOH (v/v=3:1) five times. The combined organic layers were washed with brine, dried over anhydrous Na₂SO₄, filtered and concentrated to dryness under reduced pressure. The residue was purified by prep-HPLC to give Compound 99 (30 mg, 14.9% yield) as a white solid. ¹H NMR (400 MHz, CD₃OD) δ 8.24 (d, J=1.2 Hz, 1H), 7.56 (d, J=9.2 Hz, 1H), 7.00-6.94 (m, 2H), 6.91-6.87 (m, 2H), 5.37-5.21 (m, 1H), 4.78-4.72 (m, 1H), 4.67-4.58 (m, 2H), 4.20-3.91 (m, 6H), 2.66-2.52 (m, 1H), 2.49-2.44 (m, 2H), 2.27-2.11 (m, 1H), 2.11-2.04 (m, 2H), 1.62 (d, J=7.2 Hz, 3H); LC/MS (ESI) (m/z): 554 (M+H)⁺.

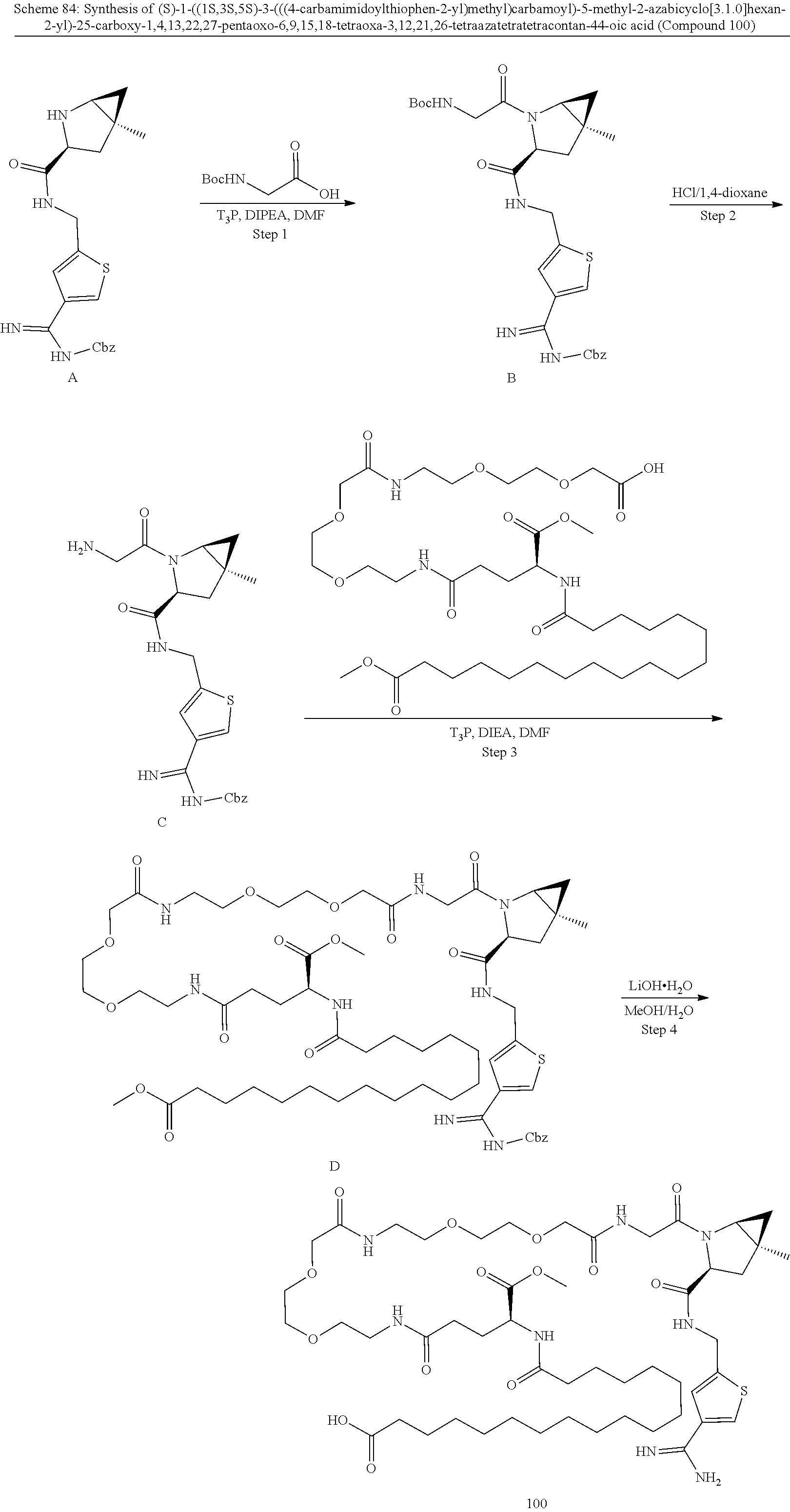

Step 1: tert-butyl (2-((1S,3S,5S)-3-(((4-(N-((benzyloxy)carbonyl)carbamimidoyl)thiophen-2-yl)methyl)carbamoyl)-5-methyl-2-azabicyclo[3.1.0]hexan-2-yl)-2-oxoethyl)carbamate (B). To a mixture of compound A (80 mg, 0.19 mmol) and [(tert-butoxycarbonyl)amino]acetic acid (44 mg, 0.25 mmol) in DMF (3 mL) was added DIPEA (0.20 mL, 1.16 mmol) and T₃P (370 mg, 0.58 mmol, 50% wt in EtOAc) at 0° C. and the mixture was stirred under N₂ atmosphere at 25° C. for 3 hours. The mixture was diluted with EtOAc and washed with saturated aq. NaHCO₃ solution. The aqueous layer was extracted with EtOAc twice. The combined organic layers were washed with brine, dried over anhydrous Na₂SO₄, filtered and concentrated to dryness under reduced pressure. The residue was purified by flash chromatography on silica gel (DCM:MeOH=20:1) to give compound B (100 mg, 90.5% yield) as a light-yellow oil. LC/MS (ESI) (m/z): 570 (M+H)⁺.

Step 2: benzyl ((5-(((1S,3S,5S)-2-glycyl-5-methyl-2-azabicyclo[3.1.0]hexane-3-carboxamido-)methyl)thiophen-3-yl)(imino)methyl)carbamate (C). A mixture of compound B (57 mg, 0.1 mmol) in HCl/1,4-dioxane (2 mL, 4 M) was stirred at room temperature for 1 hour. The reaction mixture was concentrated to dryness under reduced pressure, co-evaporated with DCM twice and dried under vacuum to give compound C (46 mg, 97.9% yield) as a white solid, which was used directly in the next step. LC/MS (ESI) m/z: 470 (M+H)⁺.

Step 3: methyl 17-{[(2S)-4-{[2-(2-{[(2-{2-[({2-[(1S,3S,5S)-3-({[4-({[(benzyloxy)carbonyl]-amino}methanimidoyl)thiophen-2-yl]methyl}carbamoyl)-5-methyl-2-azabicyclo[3.1.0]hexan-2-yl]-2-oxoethyl}carbamoyl)methoxy]ethoxy}ethyl)carbamoyl]methoxy}ethoxy)ethyl]carbamoyl}-1-methoxy-1-oxobutan-2-yl]carbamoyl}heptadecanoate (D). To a mixture of compound C (46 mg, 0.1 mmol) and (2-{2-[2-(2-{2-[(4S)-5-methoxy-4-(18-methoxy-18-oxooctadecanamido)-5-oxopentanamido]-ethoxy}ethoxy)acetamido]ethoxy}ethoxy)acetic acid (114 mg, 0.15 mmol) in DMF (3 mL) was added DIPEA (0.087 mL, 0.5 mmol) and HATU (57 mg, 0.15 mmol) at 0° C. and the mixture was stirred under N₂ atmosphere at 25° C. for 0.5 hour. The mixture was diluted with EtOAc and washed with water. The aqueous layer was extracted with EtOAc twice. The combined organic layers were washed with brine, dried over anhydrous Na₂SO₄, filtered and concentrated to dryness under reduced pressure. The residue was purified by flash chromatography on silica gel (DCM:MeOH=10:1) to give compound D (80 mg, 65.9% yield) as a white solid. LC/MS (ESI) (m/z): 1214 (M+H)⁺.

Step 4: 17-{[(1S)-3-{[2-(2-{[(2-{2-[({2-[(1S,3S,5S)-3-{[(4-carbamimidoylthiophen-2-yl)methyl]-carbamoyl}-5-methyl-2-azabicyclo[3.1.0]hexan-2-yl]-2-oxoethyl}carbamoyl)methoxy]ethoxy}ethyl)-carbamoyl]methoxy}ethoxy)ethyl]carbamoyl}-1-carboxypropyl]-carbamoyl}heptadecanoic acid (Compound 100). To a solution of compound D (80 mg, 0.066 mmol) in MeOH (1.6 mL), THE (0.4 mL) and H₂O (0.4 mL) was added LiOH·H₂O (8.4 mg, 0.2 mmol) at 0° C. and the mixture was stirred at 25° C. for 30 hours. The mixture was diluted with water and washed with MTBE twice. The aqueous layer was acidified with 0.5 M aq. HCl solution and purified by prep-HPLC to give Compound 100 (2.2 mg, 3.2% yield) as a white solid. ¹H NMR (400 MHz, CD₃OD) δ 8.25 (d, J=1.6 Hz, 1H), 7.44 (d, J=1.6 Hz, 1H), 4.79 (d, J=3.2 Hz, 1H), 4.59-4.49 (m, 2H), 4.40-4.32 (m, 2H), 4.19 (d, J=17.2 Hz, 1H), 4.05 (t, J=3.6 Hz, 2H), 4.00 (d, J=4.8 Hz, 2H), 3.73-3.53 (m, 12H), 3.45-3.40 (m, 2H), 3.40-3.35 (m, 3H), 2.44-2.10 (m, 8H), 1.66-1.57 (m, 4H), 1.33-1.27 (m, 29H), 1.19 (dd, J=5.6, 5.6 Hz, 1H), 0.82 (t, J=5.4 Hz, 1H); LC/MS (ESI) m/z: 1052 (M+H)⁺.

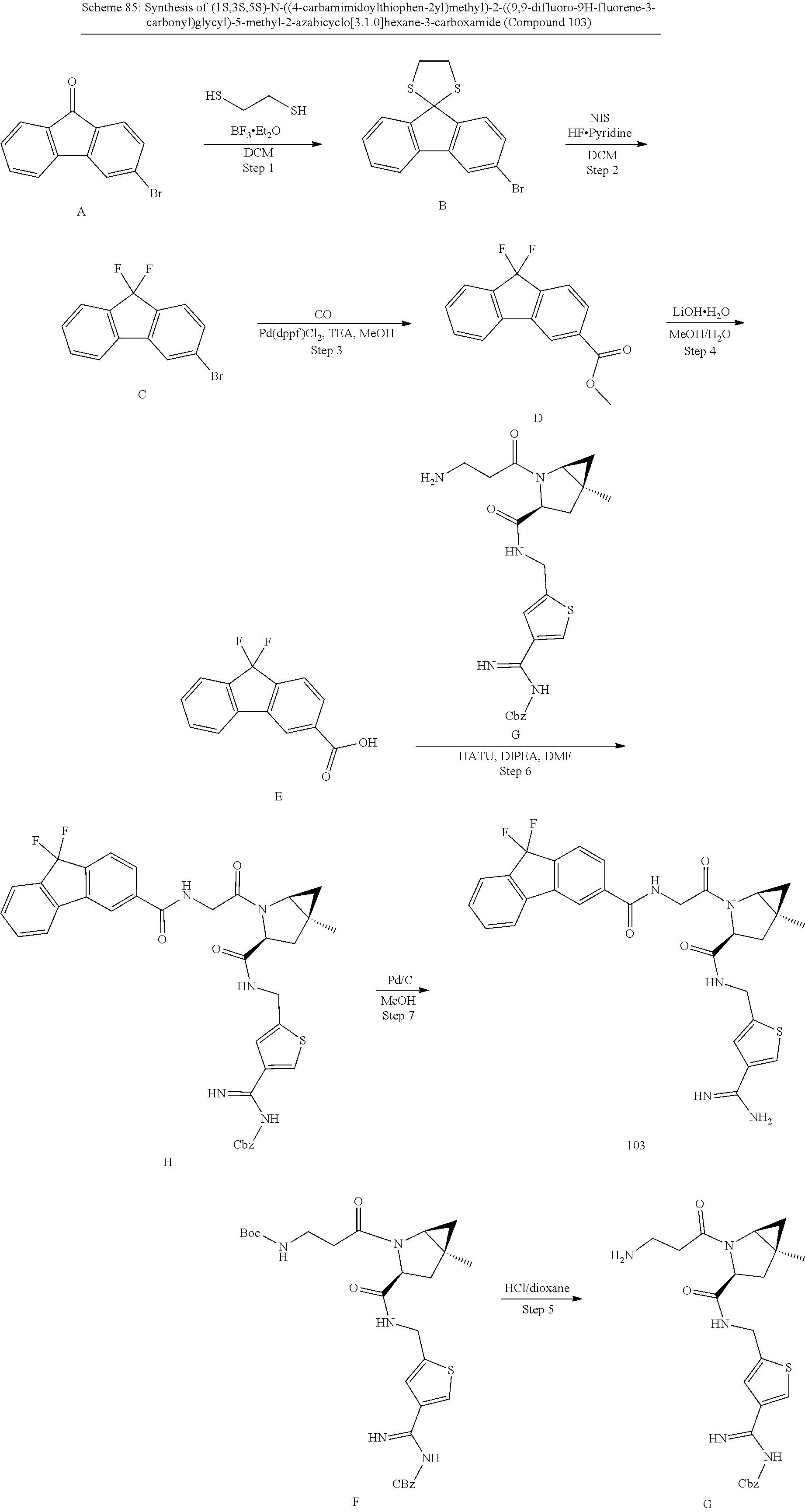

Step 1: 3-bromospiro[fluorene-9,2′-[1,3]dithiolane](B). To a solution of 3-bromo-9H-fluoren-9-one (A; 750 mg, 2.89 mmol) in dichloromethane (8 mL) was added ethane-1,2-dithiol (1.36 g, 14.5 mmol) and BF₃·Et₂O (3 mL) at 0° C. The mixture was stirred at room temperature under N₂ atmosphere for 4 hours. The mixture was diluted with water and extracted with dichloromethane (2×30 mL). The combined organic layers were washed with brine, dried over anhydrous Na₂SO₄, filtered and concentrated to dryness under reduced pressure. The residue was purified by flash chromatography on silica gel (petroleum ether:ethyl acetate=100:1 to 50:1) to give 3-bromospiro[fluorene-9,2′-[1,3]dithiolane](B; 900 mg, 92.7% yield) as a white solid.

Step 2: 3-bromo-9,9-difluoro-9H-fluorene (C). To a solution of N-iodosuccinimide (4.23 g, 18.79 mmol) in dichloromethane (5 mL) was added HF-pyridine (1 mL) at 0° C. and the mixture was stirred at room temperature for 10 minutes. A solution of 3-bromospiro[fluorene-9,2′-[1,3]dithiolane](B; 900 mg, 2.68 mmol) in dichloromethane (5 mL) was added and the mixture was stirred at room temperature for 2 hours. The reaction mixture was quenched with water and extracted with dichloromethane (2×30 mL). The combined organic layers were washed with brine, dried over anhydrous Na₂SO₄, filtered and concentrated to dryness under reduced pressure. The residue was purified by flash chromatography on silica gel (petroleum ether:ethyl acetate=100:1 to 50:1) to give 3-bromo-9,9-difluoro-9H-fluorene (660 mg, 87.5% yield) as a white solid.

Step 3: methyl 9,9-difluoro-9H-fluorene-3-carboxylate (D). To a solution of 3-bromo-9,9-difluoro-9H-fluorene (C; 330 mg, 1.17 mmol) and in methanol (10 mL) was added Pd(dppf)Cl₂ (172 mg, 0.235 mmol) and triethylamine (356 mg, 3.52 mmol). The mixture was stirred at 50° C. under CO atmosphere overnight. The reaction was quenched with saturated aq.NH₄Cl solution. The resulting mixture was extracted with ethyl acetate (3×20 mL). The combined organic layers were washed with water (2×20 mL) and brine (2×20 mL), dried over anhydrous Na₂SO₄, filtered and concentrated to dryness under reduced pressure. The residue was purified by flash chromatography on silica gel (petroleum ether:ethyl acetate=50:1 to 20:1) to give methyl 9,9-difluoro-9H-fluorene-3-carboxylate (210 mg, 68.7% yield) as a white solid.

Step 4: 9,9-difluoro-9H-fluorene-3-carboxylic acid (E). To a solution of methyl 9,9-difluoro-9H-fluorene-3-carboxylate (210 mg, 0.81 mmol) in tetrahydrofuran (6 mL) and water (2 mL) was added a solution of LiOH·H₂O (34 mg, 0.81 mmol) at 0° C. and the mixture was stirred at room temperature for 16 hours. The mixture was acidified with 1 N aq. HCl solution to pH<3 and extracted with ethyl acetate (2×10 mL). The combined organic layers were concentrated to dryness under reduced pressure to give 9,9-difluoro-9H-fluorene-3-carboxylic acid (E; 190 mg, 95.6% yield) as a white solid, which was used directly in next step without further purification. LC/MS (ESI) (m/z): 247(M+H)⁺.

Step 5: benzyl ((5-(((1S,3S,5S)-2-(3-aminopropanoyl)-5-methyl-2-azabicyclo[3.1.0]hexane-3-carboxamido)methyl)thiophen-3-yl)(imino)methyl)carbamate (G). A mixture of benzyl ((5-(((1 S,3S,5S)-2-(3-((tert-butoxycarbonyl)amino)propanoyl)-5-methyl-2-azabicyclo[3.1.0]hexane-3-carboxamido)methyl)thiophen-3-yl)(imino)methyl)carbamate (F; 47 mg, 0.092 mmol) and HCl/1,4-dioxane (1.5 mL) was stirred at room temperature for 1 hours. The reaction mixture was concentrated to dryness under reduced pressure, diluted with dichloromethane again and dried under vacuum to give benzyl ((5-(((1 S,3S,5S)-2-(3-aminopropanoyl)-5-methyl-2-azabicyclo[3.1.0]hexane-3-carboxamido)methyl)thiophen-3-yl)(imino)methyl)carbamate (G; 38.0 mg, quantitative yield) as a yellow solid, which was used directly in the next step without further purification. LC/MS (ESI) m/z: 413 (M+H)⁺.

Step 6: benzyl ((5-(((1S,3S,5S)-2-((9,9-difluoro-9H-fluorene-3-carbonyl)glycyl)-5-methyl-2-azabicyclo[3.1.0]hexane-3-carboxamido)methyl)thiophen-3-yl)(imino)methyl)carbamate (H). To a solution of 9,9-difluoro-9H-fluorene-3-carboxylic acid (E; 23 mg, 0.092 mmol) and benzyl ((5-(((1 S,3S,5S)-2-(3-aminopropanoyl)-5-methyl-2-azabicyclo[3.1.0]hexane-3-carboxamido)methyl)thiophen-3-yl)(imino)-methyl)carbamate (G; 38 mg, 0.092 mmol) in DMF (2 mL) was added T₃P (117 mg, 0.18 mmol, 50% in ethyl acetate) and DIPEA (48 mg, 0.37 mmol). The mixture was stirred at room temperature under N₂ atmosphere overnight. The reaction was quenched with saturated aq.NH₄Cl solution. The resulting mixture was extracted with ethyl acetate (3×20 mL). The combined organic layers were washed with water (2×20 mL) and brine (2×20 mL), dried over anhydrous Na₂SO₄, filtered and concentrated to dryness under reduced pressure. The residue was purified by flash chromatography on silica gel (petroleum ether:ethyl acetate=5:1 to 1:1) to give benzyl ((5-(((1S,3S,5S)-2-((9,9-difluoro-9H-fluorene-3-carbonyl)glycyl)-5-methyl-2-azabicyclo[3.1.0]hexane-3-carboxamido)methyl)thiophen-3-yl)(imino)methyl) carbamate (H; 50 mg, 77.8% yield) as a white solid. LC/MS (ESI) m/z: 698 (M+H)⁺.

Step 7: (1S,3S,5S)-N-((4-carbamimidoylthiophen-2-yl)methyl)-2-((9,9-difluoro-9H-fluorene-3-carbonyl)glycyl)-5-methyl-2-azabicyclo[3.1.0]hexane-3-carboxamide (Compound 103). To a solution of benzyl ((5-(((1 S,3S,5S)-2-((9,9-difluoro-9H-fluorene-3-carbonyl)glycyl)-5-methyl-2-azabicyclo-[3.1.0]hexane-3-carboxamido)methyl)thiophen-3-yl)(imino)methyl) carbamate (H; 50 mg, 0.072 mmol) in MeOH (2.0 mL) was added Pd/C (20 mg, 10% wt) and HCl/1,4-dioxane (cat., 2 drops) at 25° C. under N₂ atmosphere and the mixture was stirred under a H₂ balloon at 25° C. for 2 hours. The mixture was filtered and filtrate was concentrated to dryness under reduced pressure. The residue was purified by prep-HPLC to give Compound 103 (4.8 mg, 11.9% yield). ¹H NMR (400 MHz, CD₃OD) δ 8.22-8.17 (m, 2H), 7.88 (dd, J=7.8, 1.5 Hz, 1H), 7.78 (d, J=7.7 Hz, 1H), 7.73 (d, J=7.8 Hz, 1H), 7.67 (d, J=7.5 Hz, 1H), 7.60 (t, J=7.6 Hz, 1H), 7.48 (d, J=7.5 Hz, 1H), 7.42 (s, 1H), 4.85 (s, 1H), 4.58 (d, J=2.8 Hz, 2H), 4.47-4.37 (m, 2H), 3.46 (dd, J=6.0, 2.4 Hz, 1H), 2.45 (t, J=12.4 Hz, 1H), 2.20 (dd, J=13.3, 3.4 Hz, 1H), 1.33 (s, 3H), 1.21 (dd, J=5.8, 2.3 Hz, 1H), 0.85 (t, J=5.8 Hz, 1H); LC/MS (ESI) m/z: 564 (M+H)⁺.

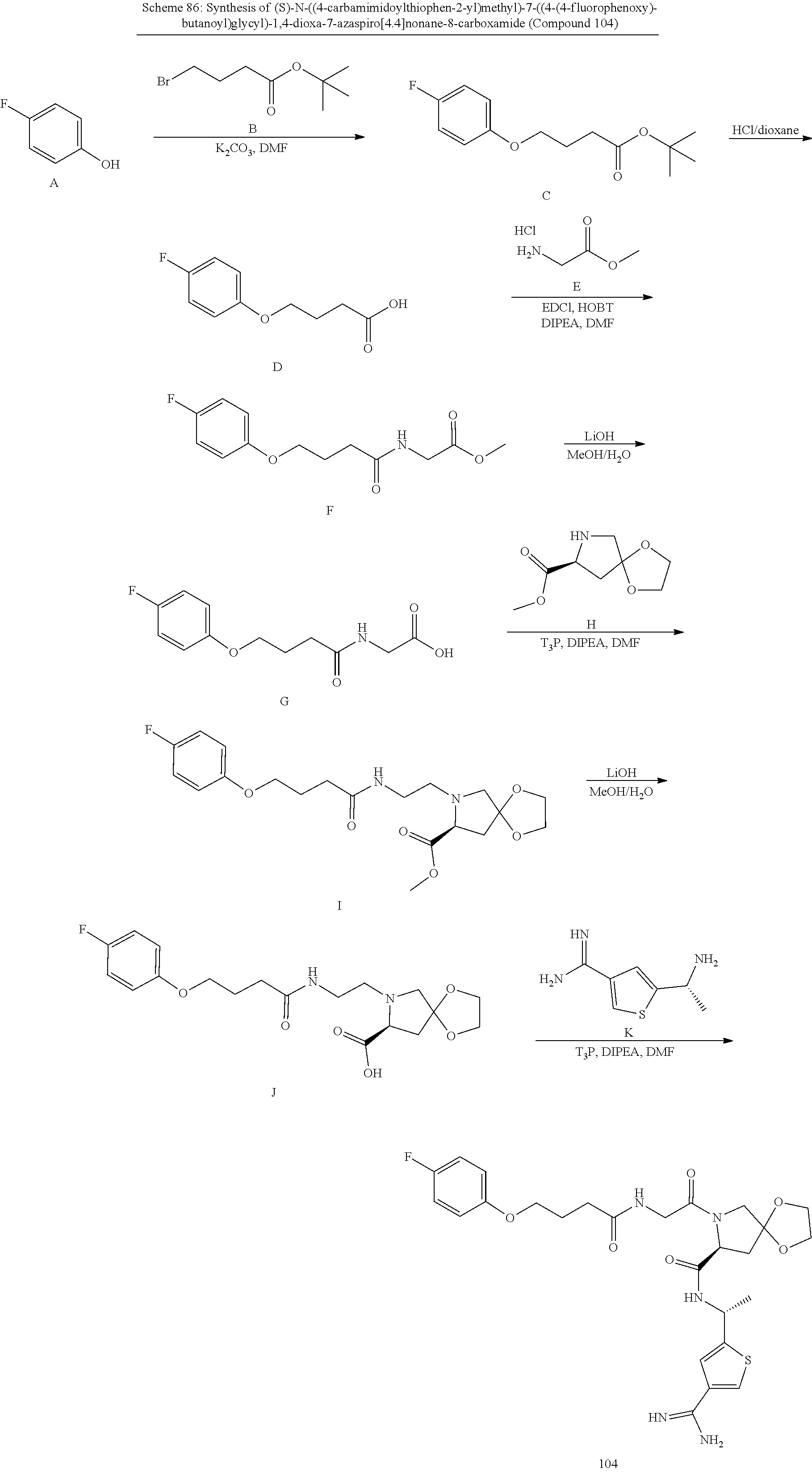

Step 1: tert-butyl 4-(4-fluorophenoxy)butanoate (C). A mixture of 4-fluorophenol (A; 1.65 g, 14.72 mmol), tert-butyl 4-bromobutanoate (B; 3.28 g, 14.72 mmol) and K₂CO₃ (14 g, 44.16 mmol) in DMF (30 mL) was added KI (2 g, 14.72 mmol). The reaction was stirred under N₂ atmosphere for 18 hours at 120° C. After cooling to room temperature, the reaction mixture was partitioned between ethyl acetate and water. The layers were separated and the organic layer was washed with brine, dried over anhydrous Na₂SO₄ and concentrated to dryness under reduced pressure to give the crude product, which was purified by flash chromatography on silica gel (petroleum ether:ethyl acetate=20:1 to 3:1) to afford tert-butyl 4-(4-fluorophenoxy)butanoate (C; 3 g, 79.7% yield) as an oil. ¹H NMR (400 MHz, CDCl₃) δ 6.96 (t, J=8.7 Hz, 2H), 6.82 (dd, J=9.1, 4.3 Hz, 2H), 3.95 (t, J=6.2 Hz, 2H), 2.42 (t, J=7.3 Hz, 2H), 2.04 (dd, J=13.6, 6.9 Hz, 2H), 1.45 (s, 9H).

Step 2: 4-(4-fluorophenoxy)butanoic acid (D). A mixture of tert-butyl 4-(4-fluorophenoxy)-butanoate (C; 3 g, 11.7 mmol) and HCl/1,4-dioxane (20 mL) was stirred at room temperature for 3 hours. The reaction mixture was concentrated to dryness under reduced pressure, diluted with DCM again and dried under vacuum to give 4-(4-fluorophenoxy)butanoic acid (D; 2 g, yield 91.4%), which was used directly in the next step without further purification. LC/MS (ESI) m/z: 199 (M+H)⁺.

Step 3: methyl (4-(4-fluorophenoxy)butanoyl)glycinate (F). To a mixture of 4-(4-fluorophenoxy)butanoic acid (D; 2.1 g, 9.09 mmol) and methyl glycinate hydrochloride (E; 1.7 g, 13.64 mmol) in DMF (15 mL) was added DIPEA (5.9 g, 15.45 mmol) and T₃P (8.7 g, 13.64 mmol, 50% in ethyl acetate) at 0° C. under N₂ atmosphere and the mixture was stirred at 35° C. for 4 hours. The mixture was quenched with saturated aq.NaHCO₃ solution and extracted with ethyl acetate twice. The combined organic layers were washed with water and brine, dried over anhydrous Na₂SO₄, filtered and concentrated to dryness under reduced pressure. The residue was purified by prep-HPLC to give methyl (4-(4-fluorophenoxy)butanoyl)glycinate (F; 2.5 g, 93.3% yield) as an oil. LC/MS (ESI) (m/z): 270 (M+H)⁺.

Step 4: (4-(4-fluorophenoxy)butanoyl)glycine (G). To a solution of methyl (4-(4-fluorophenoxy)-butanoyl)glycinate (F; 2.5 g, 9.28 mmol) in MeOH (18 mL) and water (6 mL) was added LiOH·H₂O (1.2 g, 27.85 mmol) at 0° C. and the mixture was stirred at room temperature for 3 hours. The mixture was acidified with 1 N aq. HCl solution to pH=3 and concentrated to dryness under reduced pressure to give (4-(4-fluorophenoxy)butanoyl)glycine (G; 1.8 g, 75.9% yield) as a light-yellow solid, which was used directly in next step without further purification. LC/MS (ESI) (m/z): 256 (M+H)⁺.

Step 5: methyl (S)-7-((4-(4-fluorophenoxy)butanoyl)glycyl)-1,4-dioxa-7-azaspiro[4.4]nonane-8-carboxylate (1). To a mixture of (4-(4-fluorophenoxy)butanoyl)glycine (G; 100 mg, 0.39 mmol) and methyl (S)-1,4-dioxa-7-azaspiro[4.4]nonane-8-carboxylate (H, 109 mg, 0.59 mmol) in DMF (2 mL) was added DIPEA (151, 1.171) and T₃P (372 mg, 0.59 mmol, 50% in ethyl acetate) at 0° C. under N₂ atmosphere and the mixture was stirred at 35° C. for 4 hours. The mixture was quenched with saturated aq. NaHCO₃ solution and extracted with ethyl acetate twice. The combined organic layers were washed with water and brine, dried over anhydrous Na₂SO₄, filtered and concentrated to dryness under reduced pressure. The residue was further purified by prep-HPLC to give methyl (S)-7-((4-(4-fluorophenoxy)butanoyl)glycyl)-1,4-dioxa-7-azaspiro[4.4]nonane-8-carboxylate (I; 130 mg, 78.8% yield) as an oil. LC/MS (ESI) (m/z): 425 (M+H)⁺.

Step 6: (S)-7-((4-(4-fluorophenoxy)butanoyl)glycyl)-1,4-dioxa-7-azaspiro[4.4]nonane-8-carboxylic acid (J). To a solution of methyl (S)-7-((4-(4-fluorophenoxy)butanoyl)glycyl)-1,4-dioxa-7-azaspiro[4.4]nonane-8-carboxylate (1; 130 mg, 0.31 mmol) in MeOH (1.5 mL) and water (0.5 mL) was added LiOH·H₂O (39 mg, 0.92 mmol) at 0° C. and the mixture was stirred at room temperature for 16 hours. The mixture was acidified with 1 N aq. HCl solution to pH=3 and concentrated to dryness under reduced pressure to give (S)-7-((4-(4-fluorophenoxy)butanoyl)glycyl)-1,4-dioxa-7-azaspiro[4.4]nonane-8-carboxylic acid (J; 80 mg, 77.8% yield) as a light-yellow solid, which was used directly in next step without further purification. LC/MS (ESI) (m/z): 411 (M+H)⁺.

Step 7: (S)-N-((R)-1-(4-carbamimidoylthiophen-2-yl)ethyl)-7-((4-(4-fluorophenoxy) butanoyl)-glycyl)-1,4-dioxa-7-azaspiro[4.4]nonane-8-carboxamide (Compound 104). To a mixture of (S)-7-((4-(4-fluorophenoxy)butanoyl)glycyl)-1,4-dioxa-7-azaspiro[4.4]nonane-8-carboxylic acid (J; 80 mg, 0.19 mmol) and (R)-5-(1-aminoethyl)thiophene-3-carboximidamide (60 mg, 0.29 mmol) in DMF (2 mL) was added DIPEA (126 mg, 0.97 mmol) and T₃P (248 mg, 0.39 mmol, 50% in ethyl acetate) at 0° C. under N₂ atmosphere and the mixture was stirred at 35° C. for 18 hours. The mixture was quenched with saturated aq.NaHCO₃ solution and extracted with ethyl acetate twice. The combined organic layers were washed with water and brine, dried over anhydrous Na₂SO₄, filtered and concentrated to dryness. The residue was purified by prep-HPLC to give Compound 104(15.7 mg, 14.4% yield) as a white solid. ¹H NMR (400 MHz, CD₃OD) δ 8.23 (d, J=1.6 Hz, 1H), 7.52-7.50 (m, 1H), 6.99-6.94 (m, 2H), 6.90-6.86 (m, 2H), 5.26 (q, J=6.7 Hz, 1H), 4.52 (dd, J=8.8, 6.6 Hz, 1H), 4.00-3.94 (m, 8H), 3.70 (d, J=3.3 Hz, 2H), 2.47-2.38 (m, 3H), 2.18 (dd, J=13.1, 6.5 Hz, 1H), 2.08-2.04 (m, 2H), 1.56 (d, J=7.0 Hz, 3H). LC/MS (ESI) m/z: 562 (M+H)⁺.

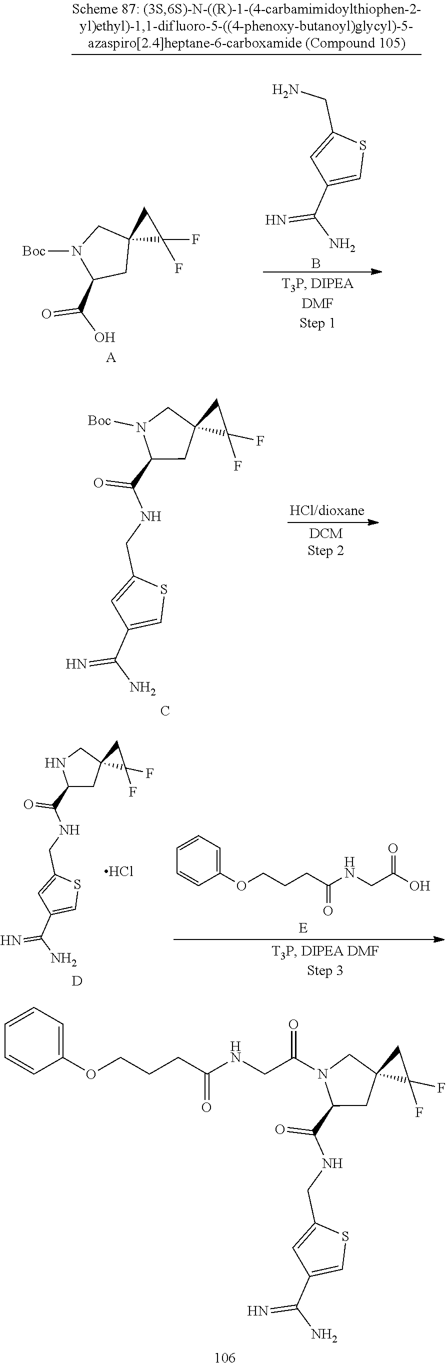

Step 1: tert-butyl (3S,6S)-6-(((R)-1-(4-carbamimidoylthiophen-2-yl)ethyl)carbamoyl)-1,1-difluoro-5-azaspiro[2.4]heptane-5-carboxylate (C). To a mixture of (3S,6S)-5-(tert-butoxycarbonyl)-1,1-difluoro-5-azaspiro[2.4]heptane-6-carboxylic acid (A; 50 mg, 0.18 mmol) and (R)-5-(1-aminoethyl)thiophene-3-carboximidamide (B; 55 mg, 0.27 mmol) in DMF (1 mL) was added DIPEA (70 mg, 0.54 mmol) and T₃P (172 mg, 0.27 mmol, 50% wt in ethyl acetate) at room temperature under N₂ atmosphere. The mixture was stirred at room temperature overnight and quenched with saturated aq.NaHCO₃ solution. The resulting mixture was extracted with ethyl acetate twice. The combined organic layers were washed with water and brine, dried over anhydrous Na₂SO₄, filtered and concentrated to dryness under reduced pressure. The residue was purified by prep-HPLC to give tert-butyl (3S,6S)-6-(((R)-1-(4-carbamimidoylthiophen-2-yl)ethyl)carbamoyl)-1,1-difluoro-5-azaspiro[2.4]heptane-5-carboxylate (C; 40 mg, 51.9% yield). LC/MS (ESI) m/z: 429 (M+H)⁺.

Step 2: (3S,6S)-N-((R)-1-(4-carbamimidoylthiophen-2-yl)ethyl)-1,1-difluoro-5-azaspiro[2.4]heptane-6-carboxamide (D). A mixture of tert-butyl (3S,6S)-6-(((R)-1-(4-carbamimidoylthiophen-2-yl)ethyl)carbamoyl)-1,1-difluoro-5-azaspiro[2.4]heptane-5-carboxylate (C; 40 mg, 0.093 mmol) and HCl/1,4-dioxane (1 mL) was stirred at room temperature for 1 hour. The reaction mixture was concentrated to dryness under reduced pressure, diluted with DCM again and dried under vacuum to give (3S,6S)-N-((R)-1-(4-carbamimidoylthiophen-2-yl)ethyl)-1,1-difluoro-5-azaspiro[2.4]heptane-6-carboxamide (D; 20 mg, yield 66.6%), which was used directly in the next step without further purification. LC/MS (ESI) m/z: 329 (M+H)⁺.

Step 3: (3S,6S)-N-((R)-1-(4-carbamimidoylthiophen-2-yl)ethyl)-1,1-difluoro-5-((4-phenoxy-butanoyl)glycyl)-5-azaspiro[2.4]heptane-6-carboxamide (Compound 105). To a mixture of (3S,6S)-N-((R)-1-(4-carbamimidoylthiophen-2-yl)ethyl)-1,1-difluoro-5-azaspiro[2.4]heptane-6-carboxamide (D; 20 mg, 0.081 mmol) and (4-phenoxybutanoyl) glycine (E; 40 mg, 0.12 mmol) in DMF (1 mL) was added DIPEA (60 mg, 0.405 mmol) and T₃P (80 mg, 0.12 mmol, 50% wt in ethyl acetate) at room temperature under N₂ atmosphere. The mixture was stirred at room temperature overnight and quenched with saturated aqueous NaHCO₃. The resulting mixture was extracted with ethyl acetate twice. The combined organic layers were washed with H₂O and brine, dried over anhydrous Na₂SO₄, filtered and concentrated to dryness under reduced pressure. The residue was purified by prep-HPLC to give Compound 105 (3.1 mg, 7.1% yield). ¹H NMR (400 MHz, CD₃OD) δ 8.23 (d, J=1.6 Hz, 1H), 7.51 (s, 1H), 7.26-7.21 (m, 2H), 6.91-6.87 (m, 3H), 5.29 (dd, J=17.7, 11.3 Hz, 1H), 4.66-4.58 (m, 1H), 4.02-3.96 (m, 4H), 3.89 (d, J=10.8 Hz, 1H), 3.70 (dd, J=10.4, 4.7 Hz, 1H), 2.58-2.45 (m, 3H), 2.12-2.04 (m, 3H), 1.58 (d, J=7.0 Hz, 3H), 1.50 (ddd, J=20.6, 10.8, 6.5 Hz, 2H); LC/MS (ESI) m/z: 548 (M+H)⁺.

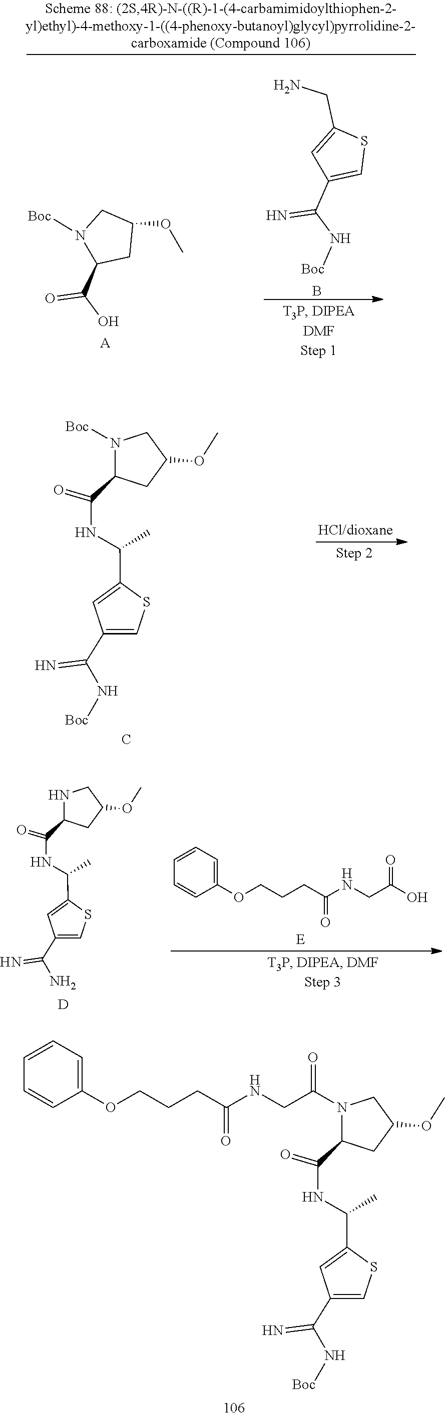

Step 1: tert-butyl (2S,4R)-2-(((R)-1-(4-(N-(tert-butoxycarbonyl)carbamimidoyl)thiophen-2-yl)ethyl)carbamoyl)-4-methoxypyrrolidine-1-carboxylate (C). To a mixture of (2S,4R)-1-(tert-butoxycarbonyl)-4-methoxypyrrolidine-2-carboxylic acid (A; 80 mg, 0.33 mmol) and benzyl ((5-(aminomethyl)thiophen-3-yl)(imino)methyl)carbamate (B; 102 mg, 0.40 mmol) in DMF (1 mL) was added DIPEA (255 mg, 1.98 mmol) and T₃P (629 mg, 0.99 mmol, 50% wt in ethyl acetate) at room temperature under N₂ atmosphere. The mixture was stirred at room temperature overnight and quenched with saturated aq.NaHCO₃ solution. The resulting mixture was extracted with ethyl acetate twice. The combined organic layers were washed with water and brine, dried over anhydrous Na₂SO₄, filtered and concentrated to dryness under reduced pressure. The residue was purified by prep-HPLC to give tert-butyl (2S,4R)-2-(((R)-1-(4-(N-(tert-butoxycarbonyl)carbamimidoyl)thiophen-2-yl)ethyl)carbamoyl)-4-methoxypyrrolidine-1-carboxylate (C; 58 mg, 35.4% yield). LC/MS (ESI) m/z: 497 (M+H)⁺.

Step 2: (2S,4R)-N-((R)-1-(4-carbamimidoylthiophen-2-yl)ethyl)-4-methoxypyrrolidine-2-carboxamide (D). A mixture of tert-butyl (2S,4R)-2-(((R)-1-(4-(N-(tert-butoxycarbonyl)carbamimidoyl) thiophen-2-yl)ethyl)carbamoyl)-4-methoxypyrrolidine-1-carboxylate (C; 58 mg, 0.12 mmol) and HCl/1,4-dioxane (1 mL) was stirred at room temperature for 1 hour. The reaction mixture was concentrated to dryness under reduced pressure, diluted with DCM again and dried under vacuum to give (2S,4R)-N-((R)-1-(4-carbamimidoylthiophen-2-yl)ethyl)-4-methoxypyrrolidine-2-carboxamide (D; 32 mg, yield 94.1%), which was used directly in the next step without further purification. LC/MS (ESI) m/z: 297 (M+H)⁺.

Step 3: (2S,4R)-N-((R)-1-(4-carbamimidoylthiophen-2-yl)ethyl)-4-methoxy-1-((4-phenoxy-butanoyl)glycyl)pyrrolidine-2-carboxamide (Compound 106). To a mixture of (2S,4R)-N-((R)-1-(4-carbamimidoylthiophen-2-yl)ethyl)-4-methoxypyrrolidine-2-carboxamide (D; 32 mg, 0.11 mmol) and (4-phenoxybutanoyl)glycine (38 mg, 0.16 mmol) in DMF (1 mL) was added DIPEA (85 mg, 0.66 mmol) and T₃P (209 mg, 0.33 mmol, 50% wt in ethyl acetate) at room temperature under N₂ atmosphere. The mixture was stirred at room temperature overnight and quenched with saturated aqueous NaHCO₃. The resulting mixture was extracted with ethyl acetate twice. The combined organic layers were washed with H₂O and brine, dried over anhydrous Na₂SO₄, filtered and concentrated to dryness. The residue was purified by prep-HPLC to give (2S,4R)-N-((R)-1-(4-carbamimidoylthiophen-2-yl)ethyl)-4-methoxy-1-((4-phenoxybutanoyl)glycyl)pyrrolidine-2-carboxamide (Compound 106; 3.7 mg, 6.5% yield). ¹H NMR (400 MHz, CD₃OD) δ 8.23 (d, J=1.6 Hz, 1H), 7.55 (dd, J=7.8, 6.6 Hz, 1H), 7.24 (dt, J=8.3, 4.7 Hz, 2H), 6.91-6.87 (m, 3H), 5.35-5.21 (m, 1H), 4.45 (t, J=8.1 Hz, 1H), 4.13-4.04 (m, 2H), 4.02-3.95 (m, 3H), 3.76-3.67 (m, 2H), 3.35 (s, 3H), 2.47 (t, J=7.4 Hz, 2H), 2.38 (ddd, J=10.5, 7.9, 2.1 Hz, 1H), 2.11-1.99 (m, 3H), 1.60 (dd, J=23.4, 7.0 Hz, 3H); LC/MS (ESI) m/z: 516 (M+H)⁺.

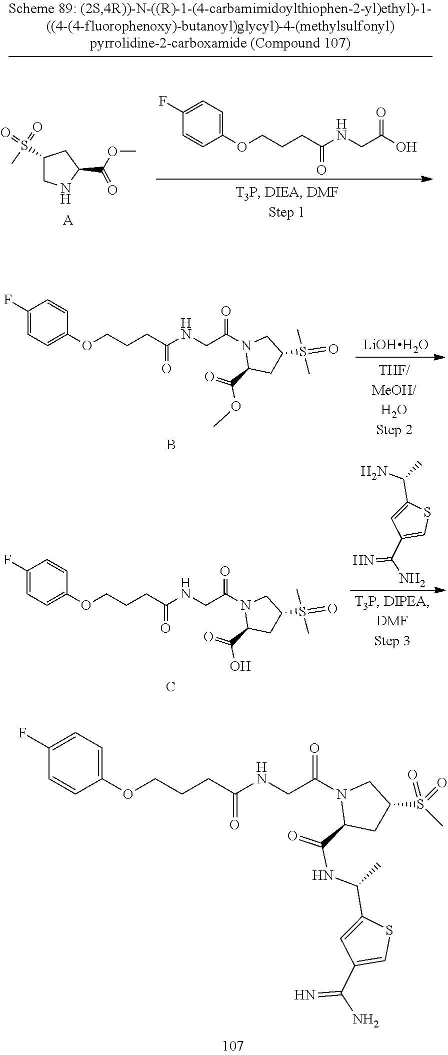

Step 1: methyl (2S,4R)-1-((4-(4-fluorophenoxy)butanoyl)glycyl)-4-(methylsulfonyl)-pyrrolidine-2-carboxylate (B). To a mixture of compound A (133 mg, 0.64 mmol) and (4-(4-fluorophenoxy)butanoyl)glycine (180 mg, 0.71 mmol) in DMF (3 mL) was added DIPEA (0.6 mL, 3.84 mmol) and T₃P (1.2 g, 1.92 mmol, 50% wt in EtOAc) at 0° C. and the mixture was stirred under N₂ atmosphere at 30° C. for 16 hours. The mixture was and diluted with EtOAc and washed with saturated aq. NaHCO₃ solution. The aqueous layer was extracted with EtOAc twice. The combined organic layers were washed with brine, dried over anhydrous Na₂SO₄, filtered and concentrated to dryness under reduced pressure. The residue was purified by flash chromatography on silica gel (DCM:MeOH=20:1) to give compound B (162 mg, yield 57%) as a yellow solid. LC/MS (ESI) m/z: 445 (M+H)⁺.

Step 2: (2S,4R)-1-((4-(4-fluorophenoxy)butanoyl)glycyl)-4-(methylsulfonyl)pyrrolidine-2-carboxylic acid (C). To a solution of compound B (162 mg, 0.36 mmol) in MeOH (1 mL), THE (0.5 mL) and H₂O (0.5 mL) was added LiOH·H₂O (15 mg, 0.36 mmol) at 0° C. and the mixture was stirred at 25° C. for 6 hours. The mixture was diluted with water and washed with EtOAc. The aqueous layer was acidified with 0.5 M aq. HCl solution and extracted with EtOAc twice. The combined organic layers were washed with brine, dried over anhydrous Na₂SO₄, filtered and concentrated to dryness under reduced pressure to give compound C (120 mg, yield 77.4%) as a yellow solid, which was used directly in the next step. LC/MS (ESI) m/z: 431 (M+H)⁺.

Step 3: (2S,4R)-N-((R)-1-(4-carbamimidoylthiophen-2-yl)ethyl)-1-((4-(4-fluorophenoxy)-butanoyl)glycyl)-4-(methylsulfonyl)pyrrolidine-2-carboxamide (Compound 107). To a mixture of compound C (120 mg, 0.28 mmol) and (R)-5-(1-aminoethyl)thiophene-3-carboximidamide(115 mg, 0.56 mmol) in DMF (3 mL) was added DIPEA (0.3 mL, 1.68 mmol) and T₃P (534 mg, 0.84 mmol, 50% wt in EtOAc) at 0° C. and the mixture was stirred under N₂ atmosphere at 35° C. for 8 hours. The mixture was diluted with CHCl₃/i-PiOH (v/v=3/1) and washed with saturated aq. NaHCO₃ solution. The aqueous layer was extracted with CHCl₃/i-PiOH (v/v=3/1) three times. The combined organic layers were dried over anhydrous Na₂SO₄, filtered and concentrated to dryness under reduced pressure. The residue was purified by prep-HPLC to give Compound 108 (13.5 mg, yield 8.3%) as a white solid 0.1H NMR (400 MHz, CD₃OD) δ 8.22 (d, J=1.6 Hz, 1H), 7.49 (s, 1H), 6.99-6.93 (m, 2H), 6.90-6.85 (m, 2H), 5.32-5.20 (m, 1H), 4.66 (dd, J=8.4, 8.4 Hz, 1H), 4.12-4.06 (m, 4H), 3.99-3.95 (m, 2H), 3.92-3.76 (m, 1H), 3.04 (d, J=14.0 Hz, 3H), 2.89-2.69 (m, 1H), 2.47-2.38 (m, 3H), 2.08-2.02 (m, 2H), 1.59 (d, J=7.2 Hz, 3H); LC/MS (ESI) m/z: 582 (M+H)⁺.

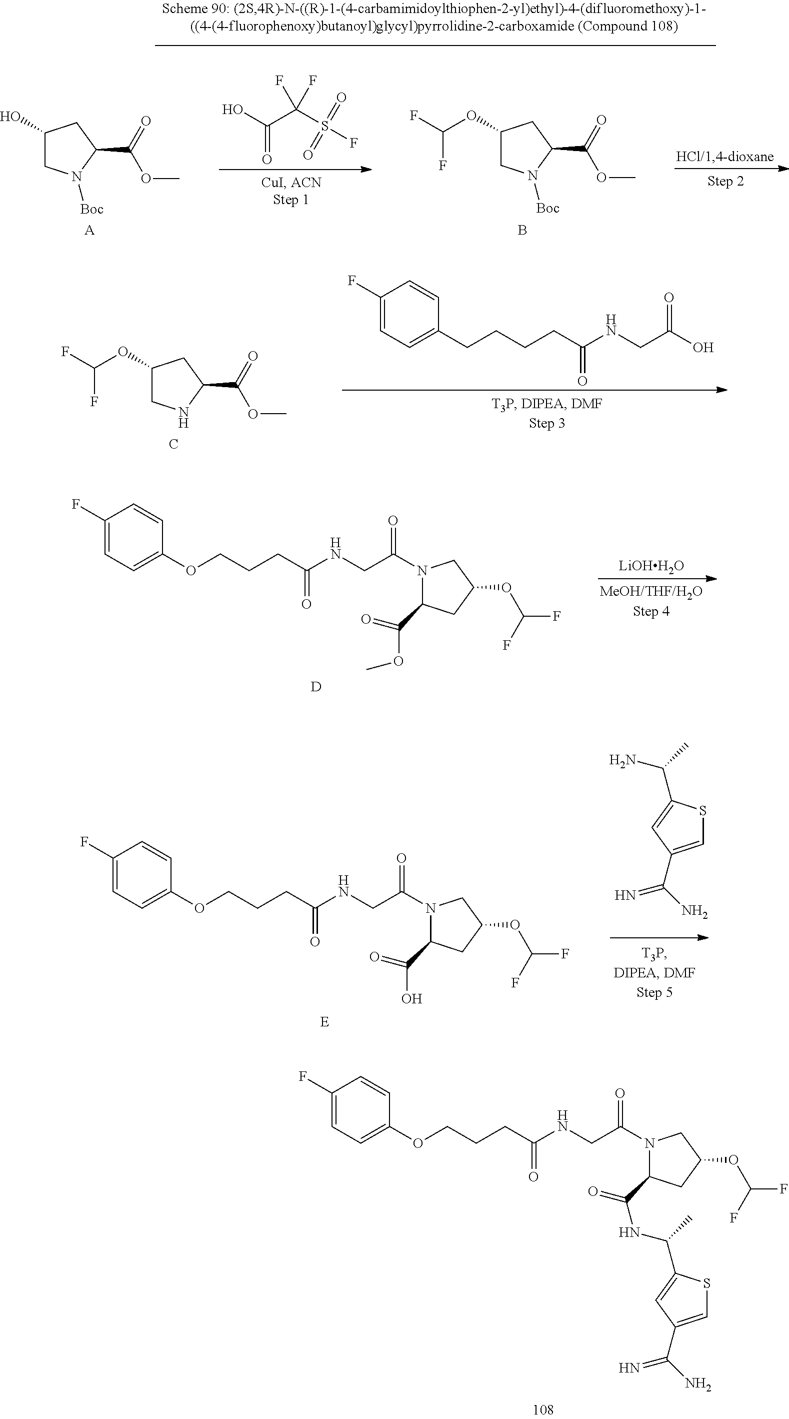

Step 1: 1-(tert-butyl) 2-methyl (2S,4R)-4-(difluoromethoxy) pyrrolidine-1,2-dicarboxylate (B). To a mixture of compound A (1 g, 4.08 mmol) in MeCN (5 mL) was added CuI (155 mg, 0.82 mmol) and a solution of 2,2-difluoro-2-(fluorosulfonyl)acetic acid (872 mg, 4.9 mmol) in MeCN (1.5 mL) at 0° C. The reaction was stirred under N₂ atmosphere at 50° C. for 16 hours. The mixture was diluted with water and extracted with EtOAc twice. The combined organic layers were washed with brine, dried over anhydrous Na₂SO₄, filtered and concentrated to dryness under reduced pressure. The residue was purified by flash chromatography on silica gel (PE:EtOAc=1:1) to give compound B (233 mg, yield 19.4%) as a yellow oil. LC/MS (ESI) m/z: 196 (M−100+H)⁺.

Step 2: methyl (2S,4R)-4-(difluoromethoxy) pyrrolidine-2-carboxylate (C). A mixture of compound B (233 mg, 0.79 mmol) in HCl/1,4-dioxane (2 mL, 4 M) was stirred at room temperature for 4 hours. The reaction mixture was concentrated to dryness, co-evaporated with DCM twice and dried under vacuum to give compound C (150 mg, yield 97.4%) as a yellow solid, which was used directly in the next step. LC/MS (ESI) m/z: 196 (M+H)⁺.

Step 3: methyl(2S,4R)-4-(difluoromethoxy)-1-((4-(4-fluorophenoxy)butanoyl)glycyl)pyrrolid ine-2-carboxylate (D). To a mixture of compound C (150 mg, 0.77 mmol) and (4-(4-fluoro-phenoxy)butanoyl)glycine (196 mg, 0.77 mmol) in DMF (3 mL) was added DIPEA (0.8 mL, 4.62 mmol) and T₃P (1.5 g, 2.31 mmol, 50% wt in EtOAc) at 0° C. and the mixture was stirred under N₂ atmosphere at 35° C. for 4 hours. The mixture was diluted with EtOAc and washed with saturated aq.NaHCO₃ solution. The aqueous layer was extracted with EtOAc twice. The combined organic layers were washed with brine, dried over anhydrous Na₂SO₄, filtered and concentrated to dryness under reduced pressure. The residue was purified by flash chromatography on silica gel (DCM:MeOH=20:1) to give compound D (155 mg, yield 46.5%) as a yellow oil. LC/MS (ESI) m/z: 433 (M+H)⁺.

Step 4: (2S,4R)-4-(difluoromethoxy)-1-((4-(4-fluorophenoxy)butanoyl)glycyl)pyrrolidine-2-carboxylic acid (E). To a solution of compound D (150 mg, 0.35 mmol) in MeOH (2 mL), THE (1 mL), and H₂O (1 mL) was added LiOH·H₂O (15 mg, 0.35 mmol) at 0° C. and the mixture was stirred at 25° C. for 4 hours. The mixture was diluted with water and washed with EtOAc. The aqueous layer was acidified with 0.5 M aq. HCl solution and extracted with EtOAc twice. The combined organic layers were dried over anhydrous Na₂SO₄, filtered and concentrated to dryness to give compound E (70 mg, yield 47.9%) as a light-yellow oil, which was used directly in the next step. LC/MS (ESI) m/z: 419 (M+H)⁺.

Step 5: (2S,4R)-N-((R)-1-(4-carbamimidoylthiophen-2-yl)ethyl)-4-(difluoromethoxy)-1-((4-(4-fluorophenoxy)butanoyl)glycyl)pyrrolidine-2-carboxamide (Compound 109). To a mixture of compound E (70 mg, 0.17 mmol) and (R)-5-(1-aminoethyl)thiophene-3-carboximidamide (70 mg, 0.34 mmol) in DMF (2 mL) was added DIPEA (0.2 mL, 1.02 mmol) and T₃P (320 mg, 0.51 mmol, 50% wt in EtOAc) at 0° C. and the mixture was stirred under N₂ atmosphere at 30° C. for 16 hours. The mixture was diluted with CHCl₃/i-PiOH (v/v=3/1) and washed with saturated aq. NaHCO₃ solution. The aqueous layer was extracted with CHCl₃/i-PiOH (v/v=3/1) three times. The combined organic layers were dried over anhydrous Na₂SO₄, filtered and concentrated to dryness under reduced pressure. The residue was purified by prep-HPLC to give Compound 108 (19.8 mg, yield 20.6%) as a white solid. ¹H NMR (400 MHz, CD₃OD) δ 8.22 (d, J=1.6 Hz, 1H), 7.53 (s, 1H), 6.98-6.94 (m, 2H), 6.89-6.85 (m, 2H), 6.49 (t, J=74.4 Hz, 1H), 5.34-5.19 (m, 1H), 5.00-4.95 (m, 1H), 4.54 (t, J=8.0 Hz, 1H), 4.07 (d, J=16.8 Hz, 1H), 3.99-3.95 (m, 3H), 3.90-3.95 (m, 1H), 3.81-3.67 (m, 1H), 2.42-3.95 (m, 3H), 2.25-2.16 (m, 1H), 2.09-2.00 (m, 2H), 1.57 (d, J=8.0 Hz, 3H); LC/MS (ESI) m/z: 570 (M+H)⁺.

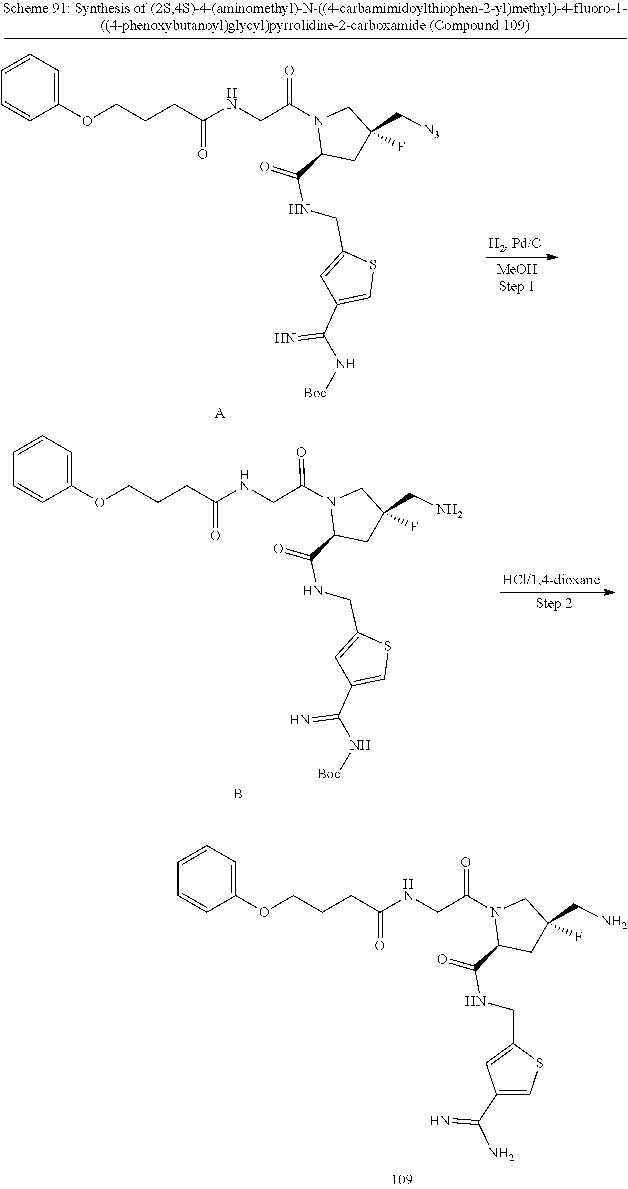

Step 1: tert-butyl ((5-(((2S,4S)-4-(aminomethyl)-4-fluoro-1-((4-phenoxybutanoyl)-glycyl)pyrrolidine-2-carboxamido)methyl)thiophen-3-yl)(imino)methyl)carbamate (B). To a solution of compound A (51 mg, 0.079 mmol) in MeOH (4 mL) was added Pd/C (10 mg, 10% wt) at 0° C. under N₂ atmosphere and the mixture was stirred under a H₂ balloon at 25° C. for 1.5 hours. The mixture was filtered and the filtrate was concentrated to dryness under reduced pressure to give compound B (30 mg, 61.3% yield) as a white solid, which was used directly in the next step. LC/MS (ESI) (m/z): 619 (M+H)⁺.

Step 2: (2S,4S)-4-(aminomethyl)-N-((4-carbamimidoylthiophen-2-yl)methyl)-4-fluoro-1-((4-phenoxybutanoyl)glycyl)pyrrolidine-2-carboxamide (Compound 110). A mixture of compound B (30 mg, 0.048 mmol) in HCl/1,4-dioxane (2 mL, 4 M) was stirred at 30° C. for 3 hours. The reaction mixture was concentrated to dryness under reduced pressure, co-evaporated with DCM twice and dried under vacuum. The residue was purified by prep-HPLC to give Compound 109 (6.6 mg, yield 26.2%) as a white solid. ¹H NMR (400 MHz, CD₃OD) δ 8.25 (d, J=1.6 Hz, 1H), 7.47 (d, J=1.6 Hz, 1H), 7.27-7.24 (m, 2H), 6.92-6.89 (m, 3H), 4.87-4.53 (m, 3H), 4.09-3.95 (m, 6H), 3.51 (dd, J=19.2, 19.2 Hz, 2H), 2.79-2.63 (m, 1H), 2.51-2.44 (m, 2H), 2.29-2.05 (m, 3H); LC/MS (ESI) (m/z): 519 (M+H)⁺.

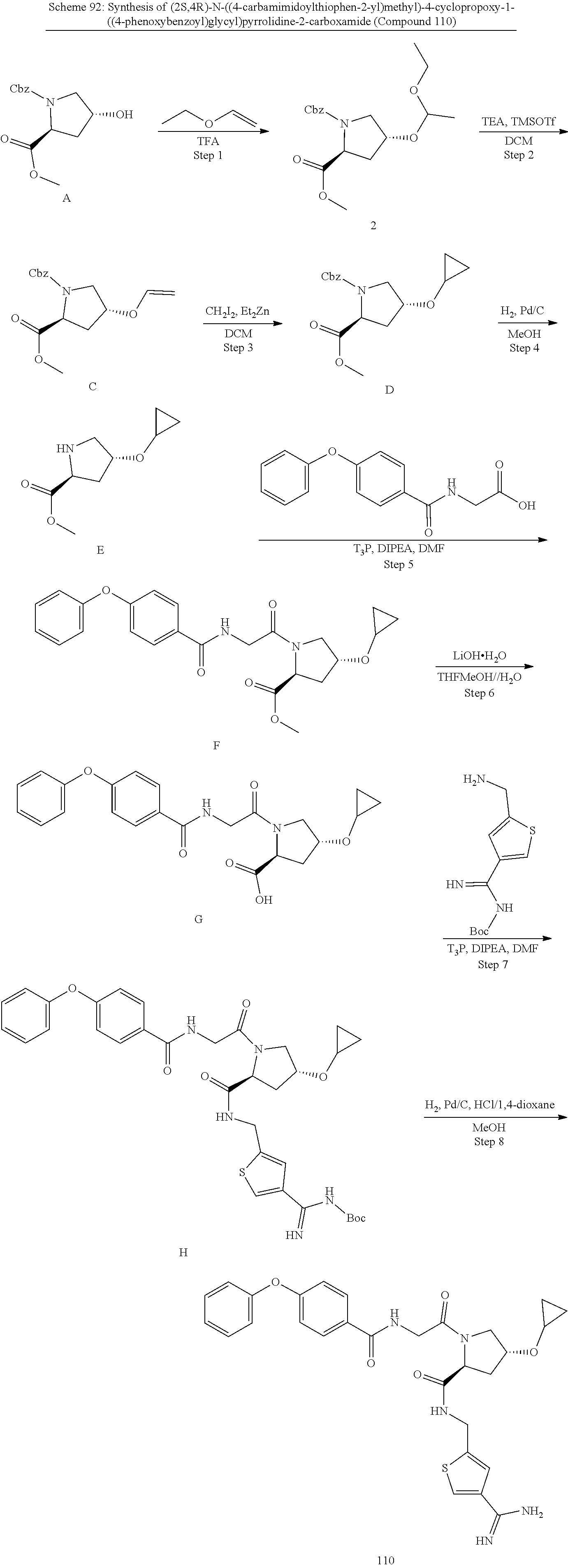

Step 1: 1-benzyl 2-methyl (2S,4R)-4-(1-ethoxyethoxy)pyrrolidine-1,2-dicarboxylate (B). To a solution of compound A (2 g, 7.16 mmol) in ethyl vinyl ether (12 mL) was added TFA (cat., 0.04 mL) dropwise at 0° C. under N₂ atmosphere and the reaction mixture was stirred under N₂ atmosphere at room temperature for 16 hours. The mixture was diluted with EtOAc and washed with saturated aq.NaHCO₃ solution. The aqueous layer was extracted with EtOAc twice and the combined organic layers were washed with brine, dried over anhydrous Na₂SO₄, filtered and concentrated to dryness under reduced pressure. The residue was purified by flash chromatography on silica gel (PE:EtOAc=5:1) to give compound B (850 mg, yield 33.8%) as a colorless oil. LC/MS (ESI) m/z: 352 (M+H)⁺.

Step 2: 1-benzyl 2-methyl (2S,4R)-4-(vinyloxy)pyrrolidine-1,2-dicarboxylate (C). To a solution of compound B (850 mg, 2.42 mmol) in DCM (10 mL) was added TEA (759 mg, 7.50 mmol) and TMSOTf (1.6 g, 7.26 mmol) dropwise at 0° C. under N₂ atmosphere and the reaction mixture was stirred under N₂ atmosphere at room temperature for 16 hours. The mixture was diluted with EtOAc and washed with water. The aqueous layer was extracted with EtOAc twice and the combined organic layers were washed with brine, dried over anhydrous Na₂SO₄, filtered and concentrated to dryness under reduced pressure. The residue was purified by flash chromatography on silica gel (PE:EtOAc=5:1) to give compound C (400 mg, yield 54.2%) as a colorless oil. LC/MS (ESI) m/z: 306 (M+H)⁺.

Step 3: 1-benzyl 2-methyl (2S,4R)-4-cyclopropoxypyrrolidine-1,2-dicarboxylate (D). To a solution of Et₂Zn (1.7 mL, 1.7 mmol, 1M in hexane) in DCM (10 mL) was added CH₂I₂ (0.13 mL, 1.61 mmol) dropwise at 0° C. under N₂ atmosphere. A solution of compound C (200 mg, 0.66 mmol) in DCM (5 mL) was added to the mixture and the reaction was stirred under N₂ atmosphere at room temperature for 16 hours. The mixture was diluted with EtOAc and washed with saturated aq. NaHCO₃ solution. The aqueous layer was extracted with EtOAc twice and the combined organic layers were washed with brine, dried over anhydrous Na₂SO₄, filtered and concentrated to dryness under reduced pressure. The residue was purified by flash chromatography on silica gel (PE:EtOAc=5:1) to give compound D (140 mg, yield 66.9%) as a colorless oil. LC/MS (ESI) m/z: 320 (M+H)⁺.

Step 4: methyl (2S,4R)-4-cyclopropoxypyrrolidine-2-carboxylate (E). To a solution of compound D (120 mg, 0.38 mmol) in MeOH (5 mL) was added Pd/C (100 mg, 10% wt) and HCl/1,4-dioxane (cat., 2 drops) at room temperature under N₂ atmosphere and the reaction mixture was stirred under a H₂ balloon at room temperature for 2 hours. The mixture was filtered and the filtrate was concentrated to dryness under reduced pressure to give compound E (65 mg, yield 93.4%) as a colorless oil. LC/MS (ESI) m/z: 186 (M+H)⁺.

Step 5: methyl (2S,4R)-4-cyclopropoxy-1-((4-phenoxybenzoyl)glycyl)pyrrolidine-2-carboxylate (F). To a mixture of compound E (65 mg, 0.35 mmol) and (4-phenoxybenzoyl)glycine (95 mg, 0.35 mmol) in DMF (2 mL) was added DIPEA (272 mg, 2.11 mmol) and T₃P (670 mg, 1.05 mmol, 50% wt in EtOAc) at 0° C. under N₂ atmosphere and the reaction mixture was stirred under N₂ atmosphere at room temperature for 3 hours. The mixture was diluted with EtOAc and washed with saturated aq.NaHCO₃ solution. The aqueous layer was extracted with EtOAc twice and the combined organic layers were washed with brine, dried over anhydrous Na₂SO₄, filtered and concentrated to dryness under reduced pressure. The residue was purified by flash chromatography on silica gel (PE:EtOAc=1:1) to give compound F (145 mg, yield 94.2%) as a white solid. LC/MS (ESI) m/z: 439 (M+H)⁺.

Step 6: (2S,4R)-4-cyclopropoxy-1-((4-phenoxybenzoyl)glycyl)pyrrolidine-2-carboxylic acid (G). To a solution of compound F (145 mg, 0.33 mmol) in THE (2 mL), MeOH (0.5 mL) and H₂O (0.5 mL) was added LiOH·H₂O (14 mg, 0.33 mmol) at 0° C. and the reaction mixture was stirred at room temperature for 16 hours. The mixture was concentrated to dryness under reduced pressure and diluted with water. The mixture was acidified with 1 N aq. HCl solution and extracted with EtOAc three times. The combined organic layers were dried over anhydrous Na₂SO₄, filtered and concentrated to dryness under reduced pressure to give compound G (100 mg, yield 71.2%) as a white solid, which was used directly in the next step. LC/MS (ESI) m/z: 425 (M+H)⁺.

Step 7: tert-butyl ((5-(((2S,4R)-4-cyclopropoxy-1-((4-phenoxybenzoyl)glycyl)pyrrolidine-2-carboxamido)methyl)thiophen-3-yl)(imino)methyl)carbamate (H). To a mixture of compound G (100 mg, 0.24 mmol) and tert-butyl ((5-(aminomethyl)thiophen-3-yl)(imino)methyl)carbamate (60 mg, 0.24 mmol) in DMF (5 mL) was added DIPEA (183 mg, 1.41 mmol) and T₃P (450 mg, 0.71 mmol, 50% wt in EtOAc) at 0° C. under N₂ atmosphere and the reaction mixture was stirred under N₂ atmosphere at room temperature for 16 hours. The mixture was diluted with EtOAc and washed with saturated aq. NaHCO₃ solution. The aqueous layer was extracted with EtOAc three times and the combined organic layers were washed with brine, dried over anhydrous Na₂SO₄, filtered and concentrated to dryness under reduced pressure. The residue was purified by flash chromatography on silica gel (PE:EtOAc=1:1) to give compound H (30 mg, yield 19.2%) as a colorless oil. LC/MS (ESI) m/z: 662 (M+H)⁺.

Step 8: (2S,4R)-N-((4-carbamimidoylthiophen-2-yl)methyl)-4-cyclopropoxy-1-((4-phenoxy-benzoyl)glycyl)pyrrolidine-2-carboxamide (Compound 110). To a solution of compound H (30 mg, 0.045 mmol) in DCM (2 mL) was added TFA (0.5 mL) dropwise at 0° C. under N₂ atmosphere and the reaction mixture was stirred under N₂ atmosphere at room temperature for 3 hours. The resulting mixture was concentrated to dryness under reduced pressure, co-evaporated with DCM twice. The residue was purified by prep-HPLC to give Compound 110 (5 mg, yield 19.7%) as a white solid. ¹H NMR (400 MHz, CD₃OD) δ 8.21 (d, J=1.6 Hz, 1H), 7.86-7.83 (m, 2H), 7.43-7.40 (m, 3H), 7.23-7.19 (m, 1H), 7.08-7.05 (m, 2H), 7.02-6.99 (m, 2H), 4.58 (s, 2H), 4.48 (t, J=8.0 Hz, 1H), 4.39-4.36 (m, 1H), 4.21 (s, 2H), 3.89-3.79 (m, 2H), 3.43-3.38 (m, 1H), 2.45-2.39 (m, 1H), 2.11-2.05 (m, 1H), 0.60-0.50 (m, 4H); LC/MS (ESI) m/z: 562 (M+H)⁺.

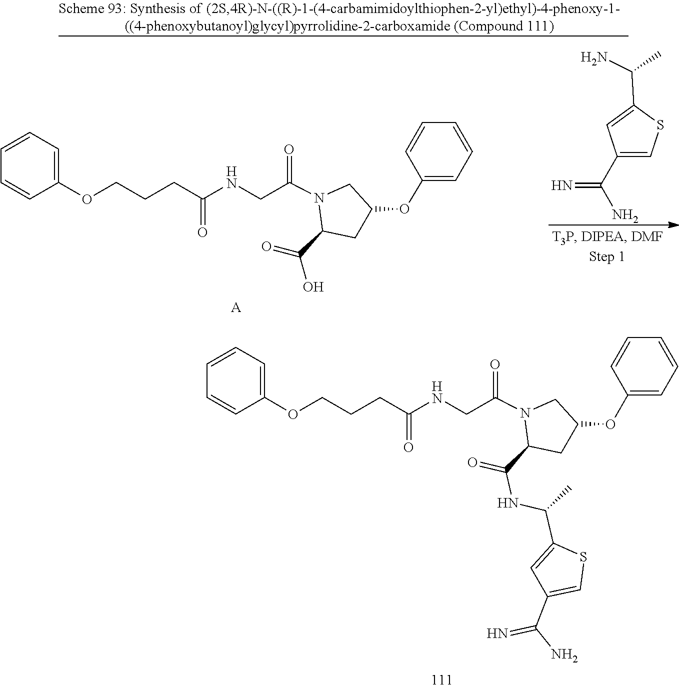

Step 1: (2S,4R)-N-((R)-1-(4-carbamimidoylthiophen-2-yl)ethyl)-4-phenoxy-1-((4-phenoxy-butanoyl)glycyl)pyrrolidine-2-carboxamide (Compound 112). To a mixture of compound A (100 mg, 0.23 mmol) and (R)-5-(1-aminoethyl)thiophene-3-carboximidamide (40 mg, 0.23 mmol) in DMF (5 mL) was added DIPEA (182 mg, 1.41 mmol) and T₃P (448 mg, 0.70 mmol, 50% wt in EtOAc) at 0° C. under N₂ atmosphere and the reaction mixture was stirred under N₂ atmosphere at room temperature for 16 hours. The resulting mixture was diluted with EtOAc and washed with saturated aq. NaHCO₃ solution. The aqueous layer was extracted with CHCl₃/i-PrOH (v/v=3/1) three times. The combined organic layers were dried over Na₂SO₄, filtered and concentrated to dryness under reduced pressure. The residue was purified by prep-HPLC to give Compound 111 (20 mg, yield 14.8%) as a white solid. ¹H NMR (400 MHz, CD₃OD) δ 8.22 (d, J=1.5 Hz, 1H), 7.57 (s, 1H), 7.32-7.21 (m, 4H), 6.99-6.87 (m, 6H), 5.29-5.23 (m, 1H), 5.13-5.08 (m, 1H), 4.60-4.55 (m, 1H), 4.05-3.85 (m, 6H), 2.56-2.51 (m, 1H) 2.47-2.44 (m, 2H), 2.23-2.19 (m, 1H), 2.10-2.03 (m, 2H), 1.57 (d, J=7.2 Hz, 3H); LC/MS (ESI) m/z: 578 (M+H)⁺.

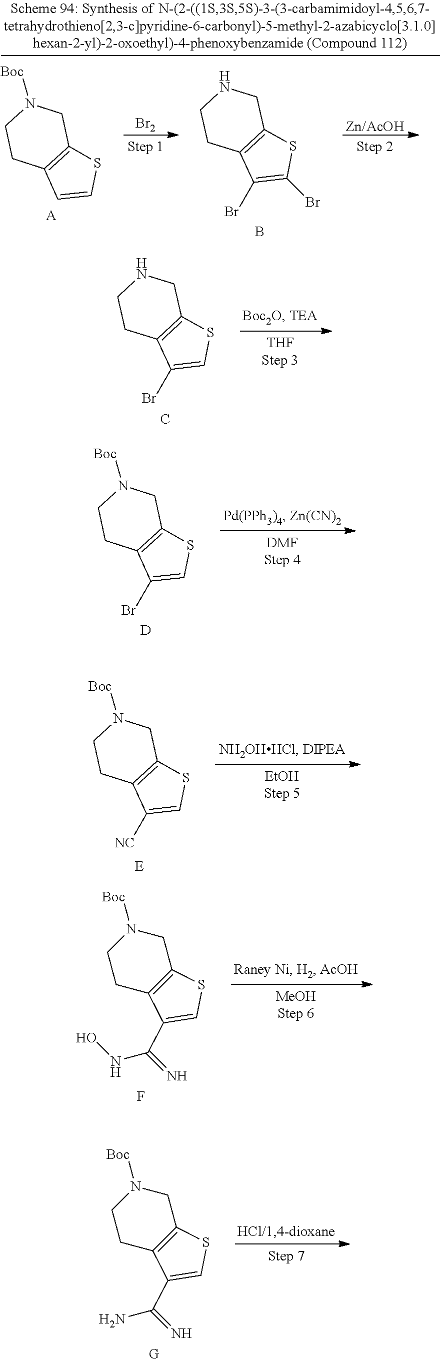

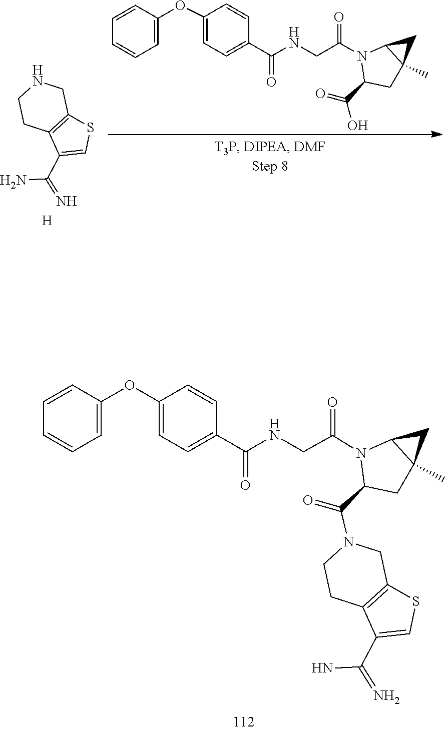

Step 1: 2,3-dibromo-4,5,6,7-tetrahydrothieno[2,3-c]pyridine (B). To a solution of compound A (3 g, 12.5 mmol) in AcOH (75 mL) was added Br₂ (10 g, 62.7 mmol) dropwise at 0° C. under N₂ atmosphere and the reaction mixture was stirred under N₂ atmosphere at 80° C. for 16 hours. The resulting mixture was filtered and concentrated to dryness under reduced pressure to give compound B (3.6 g, yield 96.7%) as a white solid. LC/MS (ESI) m/z: 298 (M+H)⁺.

Step 2: 3-bromo-4,5,6,7-tetrahydrothieno[2,3-c]pyridine (C). To a solution of compound B (1 g, 3.37 mmol) in AcOH (25 mL) was added Zinc powder (438 mg, 6.73 mmol) and 12N HCl (0.5 mL) at 0° C. under N₂ atmosphere and the reaction mixture was stirred under N₂ atmosphere at 80° C. for 16 hours. The mixture was diluted with EtOAc and washed with saturated aq.NaHCO₃ solution. The aqueous layer was extracted with EtOAc three times and the combined organic layers were washed with brine, dried over anhydrous Na₂SO₄, filtered and concentrated to dryness under reduced pressure. The residue was purified by flash chromatography on silica gel (PE:EtOAc=5:1) to give compound C (400 mg, yield 54.5%) as a white solid. LC/MS (ESI) m/z: 218/220 (M+H)⁺.

Step 3: tert-butyl 3-bromo-4,7-dihydrothieno[2,3-c]pyridine-6(5H)-carboxylate (D). To a solution of compound 3 (2.4 g, 11.0 mmol) in THE (30 mL) was added TEA (5.5 g, 55.0 mmol) and Boc₂O (3.6 g, 16.5 mmol) at 0° C. under N₂ atmosphere and the reaction mixture was stirred under N₂ atmosphere at room temperature for 16 hours. The mixture was diluted with EtOAc and washed with saturated aq.NaHCO₃ solution. The aqueous layer was extracted with EtOAc twice and the combined organic layers were washed with brine, dried over anhydrous Na₂SO₄, filtered and concentrated to dryness under reduced pressure. The residue was purified by flash chromatography on silica gel (PE:EtOAc=10:1) to give compound D (1.88 g, yield 53.7%) as a white solid. LC/MS (ESI) m/z: 318/320 (M+H)⁺.

Step 4: tert-butyl 3-cyano-4,7-dihydrothieno[2,3-c]pyridine-6(5H)-carboxylate (E). To a solution of compound D (1 g, 3.14 mmol) in DMF (15 mL) was added Zn(CN)₂ (738 mg, 6.29 mmol) and Pd(PPh₃)₄ (363 mg, 0.31 mmol) at room temperature under N₂ atmosphere and the reaction mixture was stirred under N₂ atmosphere at 80° C. for 16 hours. The mixture was diluted with water and extracted with EtOAc twice. The combined organic layers were washed with brine, dried over anhydrous Na₂SO₄, filtered and concentrated to dryness under reduced pressure. The residue was purified by flash chromatography on silica gel (PE:EtOAc=5:1) to give compound E (170 mg, yield 20.5%) as a white solid. LC/MS (ESI) m/z: 265 (M+H)⁺.

Step 5: tert-butyl 3-(N-hydroxycarbamimidoyl)-4,7-dihydrothieno[2,3-c]pyridine-6(5H)-carboxylate (F). To a solution of compound 5 (170 mg, 0.64 mmol) in EtOH (5 mL) was added hydroxylamine hydrochloride (112 mg, 1.61 mmol) and DIPEA (249 mg, 1.93 mmol) at 0° C. under N₂ atmosphere and the reaction mixture was stirred under N₂ atmosphere at room temperature for 16 hours. The mixture was diluted with EtOAc and washed with saturated aq.NaHCO₃ solution. The aqueous layer was extracted with EtOAc twice and the combined organic layers were washed with brine, dried over anhydrous Na₂SO₄, filtered and concentrated to dryness under reduced pressure. The residue was purified by flash chromatography on silica gel (PE:EtOAc=1:1) to give compound F (170 mg, yield 88.9%) as a white solid. LC/MS (ESI) m/z: 298 (M+H)⁺.

Step 6: tert-butyl 3-carbamimidoyl-4,7-dihydrothieno[2,3-c]pyridine-6(5H)-carboxylate (G). To a solution of compound F (170 mg, 0.57 mmol) in MeOH (5 mL) was added Raney Ni (1 mL, slurry in water) and AcOH (0.1 mL) at room temperature under N₂ atmosphere and the reaction mixture was stirred under a H₂ balloon at room temperature for 16 hours. The mixture was filtered and the filtrate was concentrated to dryness under reduced pressure to give compound G (160 mg, yield 99.5%) as a white solid, which was used directly in the next step. LC/MS (ESI) m/z: 282 (M+H)⁺.

Step 7: 4,5,6,7-tetrahydrothieno[2,3-c]pyridine-3-carboximidamide (H). To a solution of compound G (160 mg, 0.57 mmol) in 1,4-dioxane (2 mL) was added HCl/1,4-dioxane (2 mL) at 0° C. and the reaction mixture was stirred at room temperature for 3 hours. The mixture was concentrated to dryness under reduced pressure, co-evaporated with DCM twice and dried under vacuum to give compound H (100 mg, yield 97.0%) as a white solid, which was used directly in the next step. LC/MS (ESI) m/z: 182 (M+H)⁺.

Step 8: N-(2-((1S,3S,5S)-3-(3-carbamimidoyl-4,5,6,7-tetrahydrothieno[2,3-c]pyridine-6-carbonyl)-5-methyl-2-azabicyclo[3.1.0]hexan-2-yl)-2-oxoethyl)-4-phenoxybenzamide (Compound 112). To a mixture of compound H (37 mg, 0.20 mmol) and (1S,3S,5S)-5-methyl-2-((4-phenoxy-benzoyl)glycyl)-2-azabicyclo[3.1.0]hexane-3-carboxylic acid (40 mg, 0.10 mmol) in DMF (5 mL) was added DIPEA (79 mg, 0.61 mmol) and T₃P (194 mg, 0.30 mmol, 50% wt in EtOAc) at 0° C. under N₂ atmosphere and the reaction mixture was stirred under N₂ atmosphere at room temperature for 16 hours. The mixture was diluted with EtOAc and washed with saturated aq.NaHCO₃ solution. The aqueous layer was extracted with CHCl₃/i-PrOH (v/v=3/1) three times and the combined organic layers were dried over anhydrous Na₂SO₄, filtered and concentrated to dryness under reduced pressure. The residue was purified by prep-HPLC to give Compound 112 (4 mg, yield 7.1%) as a white solid. ¹H NMR (400 MHz, CD₃OD) δ 8.55 (s, 1H), 7.97 (s, 1H), 7.87-7.84 (m, 2H), 7.43-7.39 (m, 2H), 7.20 (t, J=7.4 Hz, 1H), 7.07-7.04 (m, 2H), 7.01-6.98 (m, 2H), 5.37-5.33 (m, 1H), 4.57-4.51 (m, 2H), 4.23-4.13 (m, 1H), 4.00-3.91 (m, 1H), 3.83-3.74 (m, 1H), 3.50-3.43 (m, 2H), 3.16-2.87 (m, 2H), 2.61-2.45 (m, 1H), 2.01 (d, J=3.8 Hz, 1H), 1.32 (d, J=6.4 Hz, 3H), 1.31-1.29 (m, 1H), 0.82-0.78 (m, 1H); LC/MS (ESI) m/z: 558 (M+H)⁺.

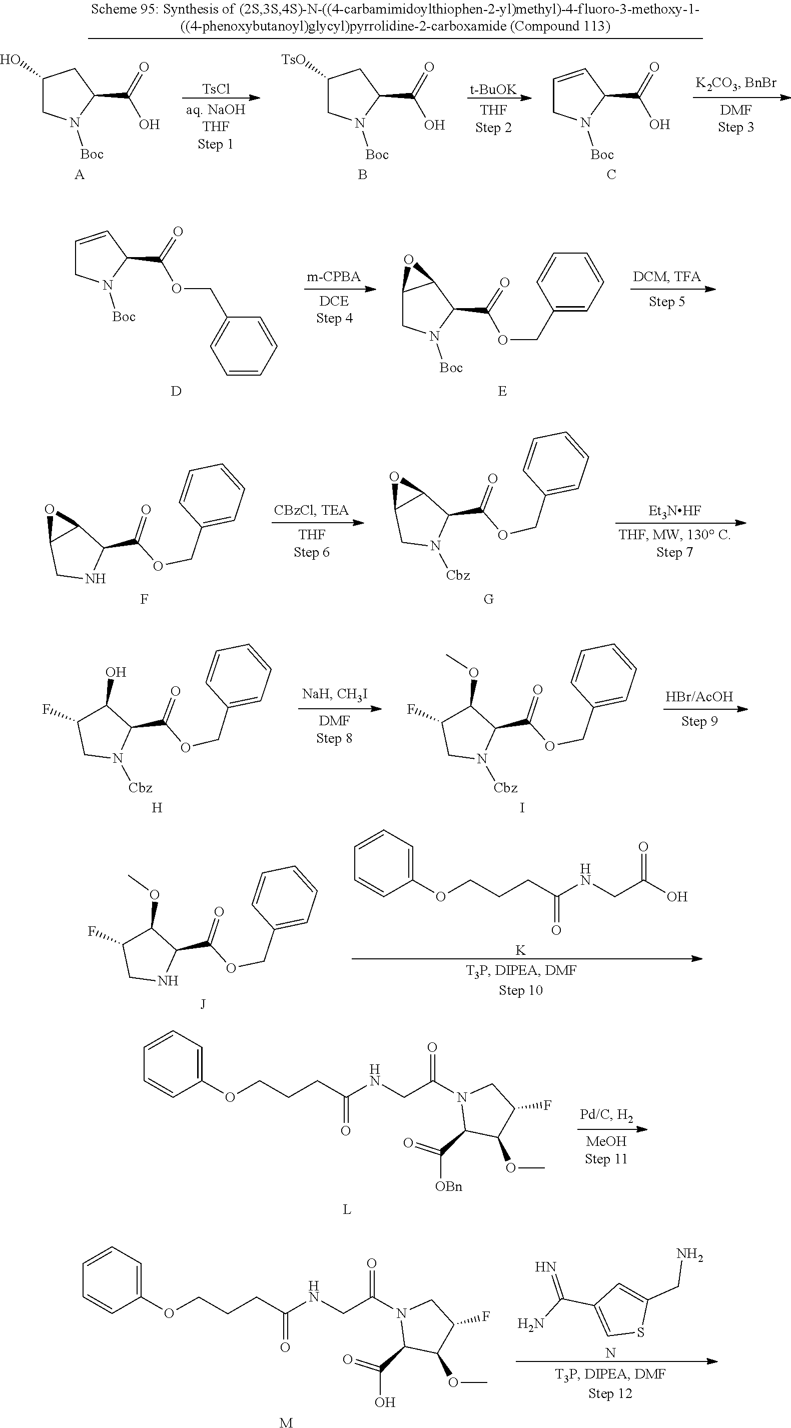

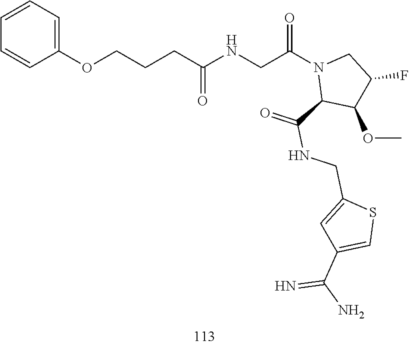 [SMILES: CO[C@H]1[C@@H](C(=O)NCc2cc(C(=N)N)cs2)N(C(=O)CNC(=O)CCCOc2ccccc2)C[C@@H]1F]

Step 1: (2S,4R)-1-(tert-butoxycarbonyl)-4-(tosyloxy)pyrrolidine-2-carboxylic acid (B). To a solution of compound A (100 g, 0.43 mol) in THE (300 mL) was added a solution of NaOH (104 g, 2.60 mol) in H₂O (400 mL) and a solution of TsCl (124 g, 0.65 mol) in THE (300 mL) respectively at 0° C. and the mixture was stirred at 25° C. for 16 hours. The mixture was concentrated to dryness under reduced pressure and diluted with water. The aqueous layer was acidified with 2 N HCl to pH˜1 and filtered. The filter cake was dried under vacuum to give compound B (164 g, yield 98.2%) as a white solid. LC/MS (ESI) m/z: 386 (M+H)⁺.

Step 2: (S)-1-(tert-butoxycarbonyl)-2,5-dihydro-1H-pyrrole-2-carboxylic acid (C). To a solution of compound B (147 g, 0.38 mol) in THE (1.8 L) was added t-BuOK (110 g, 0.95 mol) at 0° C. under N₂ atmosphere and the reaction mixture was stirred under N₂ atmosphere at room temperature for 16 hours. The mixture was diluted with water and concentrated to dryness under reduced pressure. The mixture was acidified with 2 N HCl to pH˜4 and extracted with EtOAc twice. The combined organic layers were washed with brine, dried over anhydrous Na₂SO₄, filtered and concentrated to dryness under reduced pressure to give compound C (40 g, yield 49.0%) as a yellow oil, which was used directly in next step. LC/MS (ESI) (m/z): 214 (M+H)⁺.

Step 3: 2-benzyl 1-(tert-butyl) (S)-2,5-dihydro-1H-pyrrole-1,2-dicarboxylate (D). To a solution of compound 3 (40 g, 0.19 mol) in DMF (400 mL) was added BnBr (34 mL, 0.28 mol) and K₂CO₃ (77.7 g, 0.57 mol) at 0° C. under N₂ atmosphere and the mixture was stirred under N₂ atmosphere at 25° C. for 16 hours. The mixture was diluted with water and extracted with EtOAc twice. The combined organic layers were washed with saturated aq. NH₄Cl solution, dried over anhydrous Na₂SO₄, filtered and concentrated to dryness under reduced pressure. The residue was purified by column chromatography on silica gel (PE:EtOAc=9:1) to give compound D (20.3 g, yield 35.7%) as a yellow oil. LC/MS (ESI) m/z: 304 (M+H)⁺.

Step 4: 2-benzyl 3-(tert-butyl) (1S,2S,5R)-6-oxa-3-azabicyclo[3.1.0]hexane-2,3-dicarboxylate (E). To a solution of compound D (18 g, 59.2 mmol) in DCE (180 mL) was added m-CPBA (28.3 g, 0.14 mol, 85% purity) at 0° C. under N₂ atmosphere and the mixture was stirred under N₂ atmosphere at 90° C. for 16 hours. The mixture was quenched with saturated aq.Na₂S₂O₃ solution and extracted with DCM twice. The combined organic layers were dried over anhydrous Na₂SO₄, filtered and concentrated to dryness under reduced pressure. The residue was purified by column chromatography on silica gel (PE:EtOAc=10:1 to 5:1) to give compound E (5.68 g, yield 30.1%) as a yellow oil. LC/MS (ESI) m/z: 320 (M+H)⁺.

Step 5: benzyl (1S,2S,5R)-6-oxa-3-azabicyclo[3.1.0]hexane-2-carboxylate (F). To a solution of compound 5 (5.67 g, 17.7 mmol) in DCM (40 mL) was added TFA (20 mL) at 0° C. under N₂ atmosphere and the mixture was stirred under N₂ atmosphere at 25° C. for 2 hours. The reaction mixture was concentrated to dryness under reduced pressure, co-evaporated with DCM twice and dried under vacuum to give compound 6 (3.8 g, yield 97.4%) as a brown oil, which was used directly in the next step. LC/MS (ESI) m/z: 220 (M+H)⁺.

Step 6: dibenzyl (1S,2S,5R)-6-oxa-3-azabicyclo[3.1.0]hexane-2,3-dicarboxylate (G). To a solution of compound F (3.8 g, 17.3 mmol) in THE (50 mL) was added TEA (12.0 mL, 86.5 mmol) and CbzCl (3.67 mL, 25.9 mmol) at 0° C. under N₂ atmosphere and the reaction mixture was stirred under N₂ atmosphere at 25° C. for 16 hours. The mixture was quenched with water and extracted with EtOAc twice. The combined organic layers were washed with brine, dried over anhydrous Na₂SO₄, filtered and concentrated to dryness under reduced pressure. The residue was purified by flash chromatography on silica gel (PE:EtOAc=2:1) to give compound G (3.64 g, yield 60.0%) as a brown oil. LC/MS (ESI) m/z: 354 (M+H)⁺.

Step 7: dibenzyl (2S,3S,4S)-4-fluoro-3-hydroxypyrrolidine-1,2-dicarboxylate (H). To a solution of compound G (550 mg, 1.56 mmol) in THE (1 mL) was added Et₃N·3HF (5 mL) at 0° C. under N₂ atmosphere and the mixture was microwaved under N₂ atmosphere at 120° C. for 3 hours. The mixture was quenched with saturated aq. NaHCO₃ solution and extracted with DCM twice. The combined organic layers were dried over anhydrous Na₂SO₄, filtered and concentrated to dryness under reduced pressure. The residue was purified by flash chromatography on silica gel (DCM:MeOH=20:1) to give compound G (312 mg, yield 53.7%) as a brown oil. LC/MS (ESI) m/z: 374 (M+H)⁺.

Step 8: dibenzyl (2S,3S,4S)-4-fluoro-3-methoxypyrrolidine-1,2-dicarboxylate (1). To a solution of compound H (104 mg, 0.28 mmol) in DMF (3.0 mL) was added NaH (12 mg, 0.29 mmol, 60% dispersion in mineral oil) at 0° C. under N₂ atmosphere and the mixture was stirred under N₂ atmosphere at 0° C. for 30 minutes. Then CH₃I (152 mg, 1.06 mmol) was added to the mixture at 0° C. and the mixture was stirred under N₂ atmosphere at 0° C. for another 30 minutes. The mixture was quenched with saturated aq.NH₄Cl solution and extracted with EtOAc twice. The combined organic layers were washed with brine, dried over anhydrous Na₂SO₄, filtered and concentrated to dryness under reduced pressure. The residue was purified by flash chromatography on silica gel (PE:EtOAc=9:1) to give compound I (56 mg, yield 51.8%) as a yellow oil. LC/MS (ESI) m/z: 388 (M+H)⁺.

Step 9: methyl benzyl (2S,3S,4S)-4-fluoro-3-methoxypyrrolidine-2-carboxylate (J). A solution of compound 9 (56 mg, 0.14 mmol) in HBr/AcOH (5 mL, 18% w/w) was stirred at 25° C. for 2 hours. The reaction mixture was concentrated to dryness under reduced pressure, co-evaporated with DCM twice and dried under vacuum to give compound J (30 mg, yield 82.2%) as a yellow oil, which was used directly in the next step. LC/MS (ESI) m/z: 254 (M+H)⁺.

Step 10: benzyl (2S,3S,4S)-4-fluoro-3-methoxy-1-((4-phenoxybutanoyl)glycyl)pyrrolidine-2-carboxylate (L). To a mixture of compound J (30 mg, 0.12 mmol) and (4-phenoxybenzoyl)glycine (K; 28 mg, 0.12 mmol) in DMF (3 mL) was added DIPEA (0.12 mL, 0.72 mmol) and T₃P (226 mg, 0.36 mmol, 50% wt in EtOAc) at 0° C. under N₂ atmosphere and the mixture was stirred under N₂ atmosphere at 25° C. for 16 hours. The mixture was diluted with water and extracted with CHCl₃/i-PrOH (v/v=3/1) twice. The combined organic layers were dried over anhydrous Na₂SO₄, filtered and concentrated to dryness under reduced pressure. The residue was purified by flash chromatography on silica gel (PE:EtOAc=1:1) to give compound L (25 mg, yield 41.7%) as a colorless oil. LC/MS (ESI) m/z: 473 (M+H)⁺.

Step 11: (2S,3S,4S)-4-fluoro-3-methoxy-1-((4-phenoxybutanoyl)glycyl)pyrrolidine-2-carboxylic acid (M). To a solution of compound L (25 mg, 0.05 mmol) in MeOH (5 mL) was added Pd/C (10 mg, 10% wt) at room temperature under N₂ atmosphere and the reaction mixture was stirred under a H₂ balloon at room temperature for 1.5 hours. The mixture was filtered and filtrate was concentrated to dryness under reduced pressure to give compound M (20 mg, yield 99.8%) as a colorless oil, which was used directly in next step. LC/MS (ESI) (m/z): 383 (M+H)⁺.

Step 12: (2S,3S,4S)-N-((4-carbamimidoylthiophen-2-yl)methyl)-4-fluoro-3-methoxy-1-((4-phenoxybutanoyl)glycyl)pyrrolidine-2-carboxamide (Compound 113). To a mixture of compound M (20 mg, 0.05 mmol) and 5-(aminomethyl)thiophene-3-carboximidamide (N; 12 mg, 0.075 mmol) in DMF (3 mL) was added DIPEA (0.05 mL, 0.30 mmol) and T₃P (99 mg, 0.15 mmol, 50% wt in EtOAc) at 0° C. under N₂ atmosphere and the mixture was stirred under N₂ atmosphere at 25° C. for 3 hours. The mixture was diluted with water and extracted with CHCl₃/i-PrOH (v/v=3/1) five times. The combined organic layers were dried over anhydrous Na₂SO₄, filtered and concentrated to dryness under reduced pressure. The residue was purified by prep-HPLC to give Compound 113 (5 mg, yield 18.5%) as white solid. ¹H NMR (400 MHz, CD₃OD) δ 8.21 (d, J=1.0 Hz, 1H), 7.42 (s, 1H), 7.24 (t, J=7.9 Hz, 2H), 6.90 (dd, J=7.7, 5.4 Hz, 3H), 4.84-4.83 (m, 1H), 4.68-4.59 (m, 1H), 4.56 (s, 2H), 4.24-4.18 (m, 1H), 4.04-3.82 (m, 6H), 3.39 (s, 3H), 2.48-2.44 (m, 2H), 2.10-2.03 (m, 2H); LC/MS (ESI) m/z: 520 (M+H)⁺.

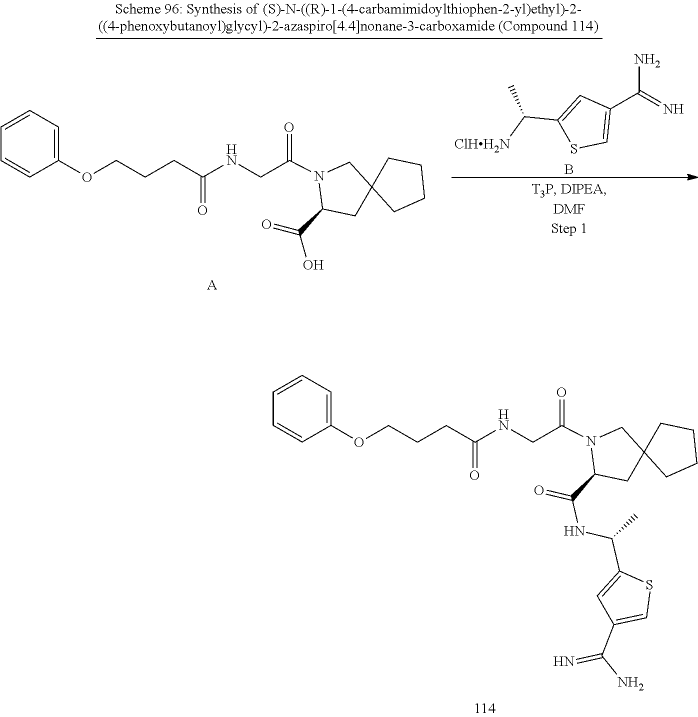

Step 1: (S)-N-((R)-1-(4-carbamimidoylthiophen-2-yl)ethyl)-2-((4-phenoxybutanoyl)glycyl)-2-azaspiro[4.4]nonane-3-carboxamide (Compound 115). To a solution of (S)-2-((4-phenoxy-butanoyl)glycyl)-2-azaspiro[4.4]nonane-3-carboxylic acid (A; 45 mg, 0.12 mmol) and (R)-5-(1-aminoethyl)thiophene-3-carboximidamide hydrochloride (B; 35 mg, 0.17 mmol) in DMF (3 mL) was added DIPEA (90 mg, 0.70 mmol) and T₃P (221 mg, 0.35 mmol, 50% in ethyl acetate). The mixture was stirred at room temperature under N₂ atmosphere overnight. The reaction was quenched with saturated aq.NH₄Cl solution. The resulting mixture was extracted with ethyl acetate (3×20 mL).

The combined organic layers were washed with water (2×20 mL) and brine (2×20 mL), dried over Na₂SO₄, filtered and concentrated to dryness under reduced pressure. The residue was purified by prep-HPLC to give Compound 114 (5.5 mg, 8.0% yield) as a white solid. ¹H NMR (400 MHz, CD₃OD) δ 8.50 (s, 1H), 8.22 (d, J=1.6 Hz, 1H), 7.55 (s, 1H), 7.24 (dd, J=13.0, 5.4 Hz, 2H), 6.91-6.87 (m, 3H), 5.25 (q, J=6.9 Hz, 1H), 4.39 (t, J=8.2 Hz, 1H), 4.10-3.89 (m, 4H), 3.49 (dd, J=40.4, 10.0 Hz, 2H), 2.46 (t, J=7.4 Hz, 2H), 2.17 (dd, J=12.5, 7.7 Hz, 1H), 2.07 (dd, J=14.0, 7.0 Hz, 2H), 1.88 (dd, J=12.4, 8.5 Hz, 1H), 1.74-1.60 (m, 7H), 1.57 (d, J=7.0 Hz, 3H), 1.52 (d, J=6.5 Hz, 1H); LC/MS(ESI) m/z: 540 (M+H)⁺.

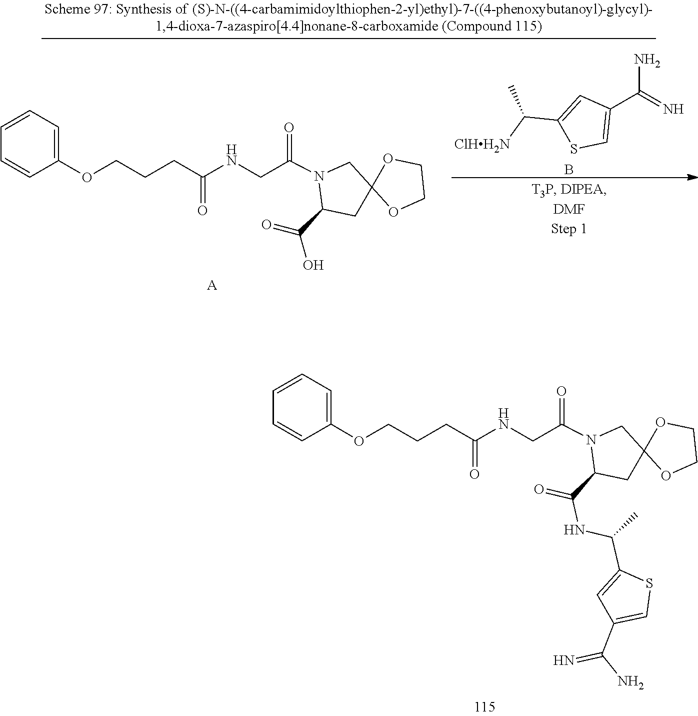

Step 1: (S)-N-((R)-1-(4-carbamimidoylthiophen-2-yl)ethyl)-7-((4-phenoxybutanoyl)glycyl)-1,4-dioxa-7-azaspiro[4.4]nonane-8-carboxamide (Compound 116). To a solution of (S)-7-((4-phenoxybutanoyl)glycyl)-1,4-dioxa-7-azaspiro[4.4]nonane-8-carboxylic acid (A; 40 mg, 0.10 mmol) and (R)-5-(1-aminoethyl)thiophene-3-carboximidamide hydrochloride (B; 32 mg, 0.15 mmol) in DMF (2 mL) was added DIPEA (79 mg, 0.61 mmol) and T₃P (194 mg, 0.31 mmol, 50% in ethyl acetate). The mixture was stirred at room temperature under N₂ atmosphere overnight. The reaction was quenched with saturated aq.NH₄Cl solution. The resulting mixture was extracted with ethyl acetate (3×20 mL). The combined organic layers were washed with water (2×20 mL) and brine (2×20 mL), dried over Na₂SO₄, filtered and concentrated to dryness under reduced pressure. The residue was purified by prep-HPLC to give Compound 115 (1.9 mg, 3.4% yield) as a white solid. ¹H NMR (400 MHz, CD₃OD) δ 8.49 (s, 1H), 8.22 (d, J=1.2 Hz, 1H), 7.51 (s, 1H), 7.22 (td, J=7.6, 2.1 Hz, 2H), 6.91-6.85 (m, 3H), 5.25 (d, J=6.9 Hz, 1H), 4.51 (dd, J=8.8, 6.6 Hz, 1H), 4.02-3.90 (m, 8H), 3.73-3.65 (m, 2H), 2.49-2.33 (m, 3H), 2.17 (dd, J=13.1, 6.5 Hz, 1H), 2.10-2.02 (m, 2H), 1.57 (t, J=14.0 Hz, 3H); LC/MS(ESI) m/z: 544 (M+H)⁺.

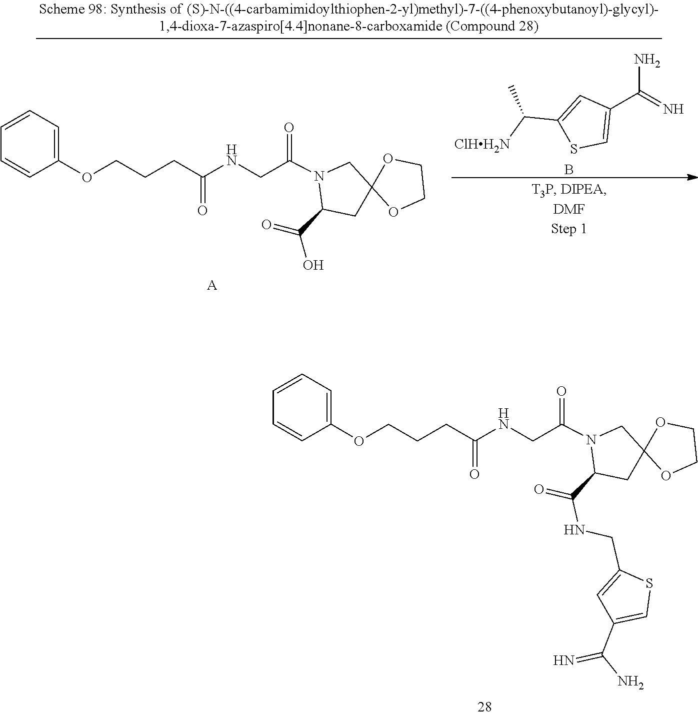

Step 1: (S)-N-((4-carbamimidoylthiophen-2-yl)methyl)-7-((4-phenoxybutanoyl)glycyl)-1,4-dioxa-7-azaspiro[4.4]nonane-8-carboxamide (Compound 28). To a solution of (S)-7-((4-phenoxy-butanoyl)glycyl)-1,4-dioxa-7-azaspiro[4.4]nonane-8-carboxylic acid (A; 85 mg, 0.22 mmol) and 5-(aminomethyl)thiophene-3-carboximidamide hydrochloride (B; 62 mg, 0.33 mmol) in DMF (5 mL) was added DIPEA (168 mg, 1.30 mmol) and T₃P (413 mg, 0.65 mmol, 50% in ethyl acetate). The mixture was stirred at room temperature under N₂ atmosphere overnight. The reaction was quenched with saturated aq.NH₄Cl solution. The resulting mixture was extracted with ethyl acetate (3×20 mL).

The combined organic layers were washed with water (2×20 mL) and brine (2×20 mL), dried over anhydrous Na₂SO₄, filtered and concentrated to dryness under reduced pressure. The residue was purified by prep-HPLC to give (S)-N-((4-carbamimidoylthiophen-2-yl)methyl)-7-((4-phenoxybutanoyl)glycyl)-1,4-dioxa-7-azaspiro-[4.4]nonane-8-carboxamide (18 mg, 15.7% yield) as a white solid. ¹H NMR (400 MHz, CD₃OD) δ 8.55 (s, 1H), 8.22 (t, J=3.1 Hz, 1H), 7.42 (s, 1H), 7.24 (dd, J=9.4, 6.7 Hz, 2H), 6.92-6.87 (m, 3H), 4.55 (s, 3H), 4.02-3.88 (m, 8H), 3.70 (s, 2H), 2.50-2.37 (m, 3H), 2.18 (dd, J=13.2, 5.4 Hz, 1H), 2.10-2.04 (m, 2H); LC/MS(ESI) m/z: 530 (M+H)⁺.

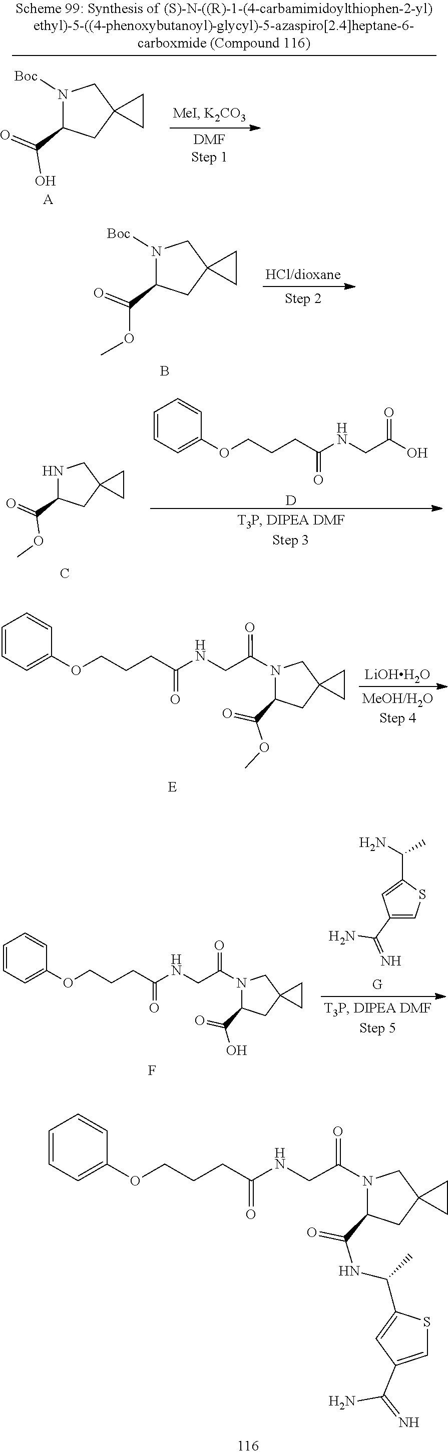

Step 1: 5-(tert-butyl) 6-methyl (S)-5-azaspiro[2.4]heptane-5,6-dicarboxylate (B). To a solution of (S)-5-(tert-butoxycarbonyl)-5-azaspiro[2.4]heptane-6-carboxylic acid (A; 500 mg, 2.08 mmol) and K₂CO₃ (856 mg, 6.22 mmol) in DMF (5 mL) was added iodomethane (441 mg, 3.11 mmol) at 0° C. and the mixture was stirred at room temperature for 4 hours. The mixture was acidified with 1 N aq. HCl solution to pH<3 and extracted with ethyl acetate (2×10 mL). The combined organic layers were concentrated to dryness under reduced pressure. The residue was purified by flash chromatography on silica gel (petroleum ether:ethyl acetate=20:1 to 5:1) to give 5-(tert-butyl) 6-methyl (S)-5-azaspiro[2.4]heptane-5,6-dicarboxylate (449 mg, 84.6% yield) as a colorless oil. LC/MS (ESI) (m/z): 256 (M+H)⁺.

Step 2: methyl (S)-5-azaspiro[2.4]heptane-6-carboxylate (C). A solution of 5-tert-butyl 6-methyl (6S)-5-azaspiro[2.4]heptane-5,6-dicarboxylate (449 mg, 1.76 mmol) in 4 N HCl/1,4-dioxane (3 mL) was stirred at 25° C. for 1 hour. The mixture was concentrated to dryness to give methyl (S)-5-azaspiro[2.4]heptane-6-carboxylate (C; 375 mg, 83.5% yield) as a white solid, which was used directly without purification. LC/MS (ESI) (m/z): 156 (M+H)⁺.

Step 3: methyl 5-[2-(4-phenoxybutanamido)acetyl]-5-azaspiro[2.4]heptane-6-carboxylate (E). To a solution of (4-phenoxybutanamido)acetic acid (D; 308 mg, 1.30 mmol) and methyl (S)-5-azaspiro[2.4]heptane-6-carboxylate (C; 372 mg, 1.95 mmol) in dimethylformamide (3 mL) was added N,N-diisopropylethylamine (504 mg, 1.30 mmol) and T₃P (1.24 g, 1.95 mmol). The mixture was stirred at room temperature under nitrogen atmosphere overnight. The reaction was quenched with saturated aqueous NH₄Cl. The resulting mixture was extracted with ethyl acetate (3×100 mL). The combined organic layers were washed with water (2×100 mL) and brine (2×100 mL), dried over anhydrous Na₂SO₄, filtered and concentrated to dryness under reduced pressure. The residue was purified by column chromatography on silica gel (PE/EtOAc=5:1 to 1:1) to give methyl 5-[2-(4-phenoxybutanamido)acetyl]-5-azaspiro[2.4]-heptane-6-carboxylate (275 mg, 56.6% yield) as a light-yellow oil. LC/MS (ESI) (m/z): 375 (M+H)⁺.

Step 4: 5-[2-(4-phenoxybutanamido)acetyl]-5-azaspiro[2.4]heptane-6-carboxylic acid (F). To a solution of methyl 5-[2-(4-phenoxybutanamido)acetyl]-5-azaspiro[2.4]heptane-6-carboxylate (E; 275 mg, 0.73 mmol) in methanol (3 mL) and water (1 mL) was added a solution of LiOH (92 mg, 2.20 mmol) at 0° C. and the mixture was stirred at room temperature for 16 hours. The mixture was acidified with 1 N aq. HCl to pH<3 and concentrated to dryness under reduced pressure to give 5-[2-(4-phenoxybutanamido)acetyl]-5-azaspiro[2.4]heptane-6-carboxylic acid (F; 91 mg, 34.4% yield) as a white solid, which was used directly in next step without further purification. LC/MS (ESI) (m/z): 361(M+H)⁺.

Step 5: (6S)-N-[(1R)-1-(4-carbamimidoylthiophen-2-yl)ethyl]-5-[2-(4-phenoxybutanamido) acetyl]-5-azaspiro[2.4]heptane-6-carboxamide (Compound 116). To a solution of 5-[2-(4-phenoxybutanamido)acetyl]-5-azaspiro[2.4]heptane-6-carboxylic acid (F; 91 mg, 0.25 mmol) and (R)-5-(1-aminoethyl)thiophene-3-carboximidamide (G; 64 mg, 0.38 mmol) in dimethylformamide (1.5 mL) was added DIPEA (126 mg, 0.97 mmol) and T₃P (241 mg, 0.38 mmol, 50% in ethyl acetate). The mixture was stirred at room temperature under N₂ atmosphere overnight. The mixture was quenched with saturated aq.NaHCO₃ solution and extracted with ethyl acetate twice. The combined organic layers were washed with water and brine, dried over anhydrous Na₂SO₄, filtered and concentrated to dryness under reduced pressure. The residue was purified by prep-HPLC to give Compound 116 (1.8 mg, 1.4% yield) as a white solid. ¹H NMR (400 MHz, CD₃OD) δ 8.24 (d, J=1.5 Hz, 1H), 7.52 (d, J=5.0 Hz, 1H), 7.23 (dd, J=9.6, 6.3 Hz, 2H), 6.91-6.87 (m, 3H), 5.31-5.26 (m, 1H), 4.58 (dd, J=8.6, 4.2 Hz, 1H), 4.02-3.96 (m, 4H), 3.61 (d, J=9.7 Hz, 1H), 3.50 (d, J=9.7 Hz, 1H), 2.47 (t, J=7.4 Hz, 2H), 2.28 (dd, J=12.7, 8.6 Hz, 1H), 2.11-2.05 (m, 2H), 1.84 (dd, J=12.8, 4.2 Hz, 1H), 1.60 (t, J=13.3 Hz, 3H), 0.68-0.60 (m, 3H), 0.55 (dd, J=12.6, 5.7 Hz, 1H); LC/MS (ESI) (m/z): 512(M+H)⁺.

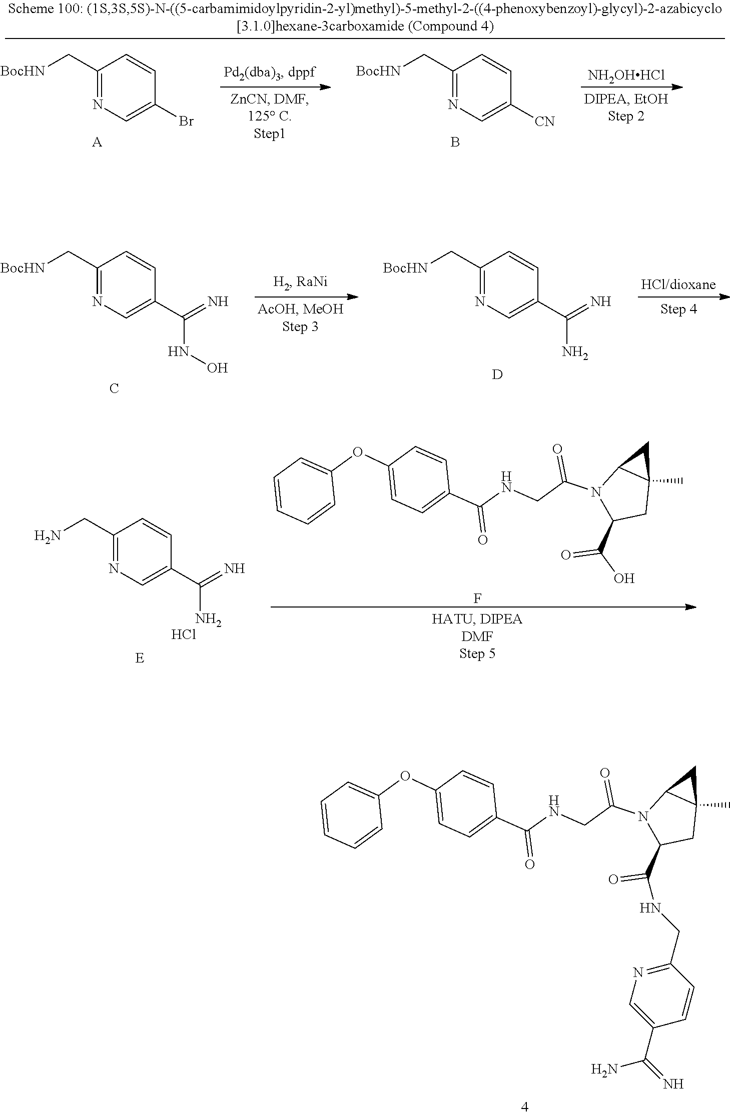

Step 1: tert-butyl N-[(5-cyanopyridin-2-yl)methyl]carbamate (B). A solution of tert-butyl N-[(5-bromopyridin-2-yl)methyl]carbamate (A; 1.0 g, 3.48 mmol, 1.0 equiv.), ZnCN ((0.61 g, 5.22 mmol, 1.5 equiv.), tris(dibenzylideneacetone) dipalladium (0) (0.32 g, 0.35 mmol, 0.1 equiv.), 1,1′-bis(diphenylphosphino) ferrocene (0.39 g, 0.70 mmol, 0.2 equiv.) in DMF (6 mL) was heated to 125° C. and stirred for 1.5 hours. The reaction was cooled to room temperature and then diluted with water and EtOAc. The two layers were separated, and the aqueous layer was extracted with EtOAc. The combined organic extracts were washed with brine, dried over Na₂SO₄ and concentrated to give a brown oil. Purification by Combi-Flash; 40 g column, solvent A=hexanes, solvent B=EtOAc. 100% A to 30% B gave compound B (0.8 g, 3.43 mmol, Yield 98.48%) as a brown sticky oil. LC/MS (ESI) m/z: 234 (M+H)⁺.

Step 2: tert-butyl N-[(5-(N-hydroxycarbamimidoyl)pyridin-2-yl]methyl) carbamate (C). To a mixture of compound B (0.8 g, 3.43 mmol, 1.0 equiv.) and hydroxylamine hydrochloride (0.596 g, 8.57 mmol, 2.5 equiv.) in ethanol (5 mL) was added DIPEA (1.8 mL, 10.29 mmol, 3.0 equiv.). The reaction was stirred at room temperature overnight, concentrated to dryness and the residue diluted with water and CH₂Cl₂. The two layers were separated, and the aqueous layer was extracted with CH₂Cl₂. The combined organic extracts were washed with brine, dried over Na₂SO₄ and concentrated. Purification by Combi-Flash; 24 g column; solvent A=CH₂Cl₂, solvent B=MeOH. 100% A to 5% B gave compound C (0.4 g, 1.51 mmol, Yield 43.80%) as a brown solid. LC/MS (ESI) m/z: 267 (M+H)⁺.

Step 3: tert-butyl N-[(5-carbamimidoylpyridin-2-yl)methyl]carbamate (D). To a solution of compound C (0.4 g, 1.50 mmol, 1.0 equiv.) in MeOH (5 mL) was added acetic acid (0.5 mL) and Raney-Ni (0.088 g, 1.50 mmol, 0.1 equiv.). The flask was evacuated and then back filled with hydrogen gas in a balloon. The reaction was stirred at 30° C. for 16 hours, filtered through Celite pad, and concentrated to obtain compound D (0.3 g, 1.50 mmol, Yield 79.79%) as a yellow solid, which was used in the next step without further purification. LC/MS (ESI) m/z: 251 (M+H)⁺.

Step 4: 6-(aminomethyl)pyridine-3-carboximidamide. HCl (E). A solution of compound D (0.3 g, 1.2 mmol, 1.0 equiv.) in HCl/1,4-dioxane (3 mL, 4M) was stirred at room temperature for 3 hours. The reaction mixture was concentrated to dryness to give compound E (172 mg, 1.15 mmol, Yield 95.55%) as a yellow solid, which was used directly in the next step without further purification. LC/MS (ESI) m/z: 151 (M+H)⁺.

Step 5: 1S,3S,5S)-N-[(5-carbamimidoylpyridin-2-yl)methyl]-5-methyl-2-{2-[(4-phenoxy-phenyl)formamido]acetyl}-2-azabicyclo[3.1.0]hexane-3-carboxamide (Compound 4). To a mixture of compound E (0.01 g, 0.025 mmol, 1.2 equiv.) and (1S,3S,5S)-5-methyl-2-{2-[(4-phenoxyphenyl)-formamido]acetyl}-2-azabicyclo[3.1.0]hexane-3-carboxylic acid (F; 0.006 g, 0.03 mmol, 1.0 equiv.) in DMF (1 mL) was added HATU (0.012 g, 0.03 mmol, 1.5 equiv.), followed by DIPEA (0.02 mL, 0.10 mmol, 4.0 equiv.). The reaction was stirred at room temperature for 0.5 hours and then purified directly using prep-HPLC to give Compound 4 (6 mg, 0.038 mmol, Yield 44.94%) as a white solid. ¹H NMR (400 MHz, DMSO-d₆) δ 8.87 (d, J=9.3 Hz, 1H), 8.72 (t, J=5.6 Hz, 1H), 8.65-8.53 (m, 1H), 8.42 (s, 1H), 8.11 (dd, J=25.8, 8.2 Hz, 1H), 7.88 (d, J=8.4 Hz, 2H), 7.53-7.40 (m, 3H), 7.22 (t, J=7.4 Hz, 1H), 7.06 (dd, J=26.8, 8.1 Hz, 2H), 4.75 (dd, J=11.5, 3.2 Hz, 1H), 4.51-4.32 (m, 3H), 4.04 (dd, J=16.4, 5.5 Hz, 1H), 3.50-3.43 (m, 1H), 2.33 (t, J=12.4 Hz, 1H), 2.03 (dd, J=13.2, 3.1 Hz, 1H), 1.23 (d, J=11.7 Hz, 4H), 0.69 (t, J=5.7 Hz, 1H). LC/MS (ESI) m/z: 527 (M+H)⁺.

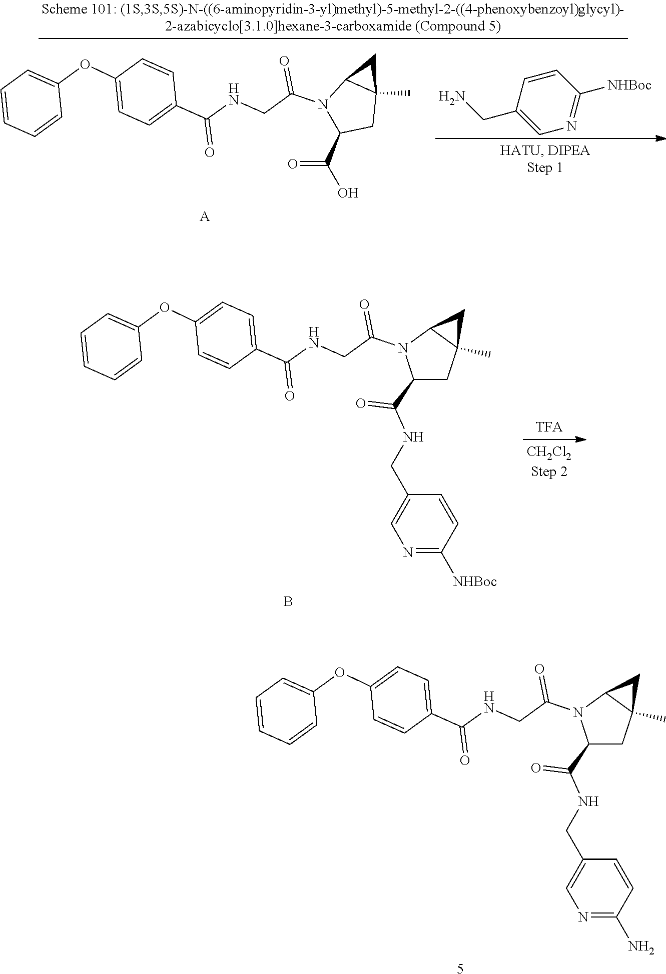

Step 1: tert-butyl N-[5-({[(1S,3S,5S)-5-methyl-2-{2-[(4-phenoxyphenyl) formamido]acetyl}-2-azabicyclo[3.1.0]hexan-3-yl]formamido}methyl)pyridin-2-yl]carbamate (B). To a mixture of compound 1 (15 mg, 0.038 mmol, 1.0 equiv.) and tert-butyl N-[5-(aminomethyl)pyridin-2-yl]carbamate (10 mg, 0.05 mmol, 1.2 equiv.) in DMF (1 mL) was added HATU (22 mg, 0.057 mmol, 1.3 equiv.), followed by DIPEA (0.03 mL, 0.15 mmol, 4.0 equiv.). The reaction was stirred at room temperature for 30 minutes and then diluted with water (1 mL). The cloudy mixture was filtered, and the residue was dissolved in CH₂Cl₂, washed with brine, dried over Na₂SO₄ and concentrate d to give compound B as a white solid, which was used in the next reaction without further purification. LC/MS (ESI) m/z: 600 (M+H)⁺.

Step 2: 1S,3S,5S)-N-[(6-aminopyridin-3-yl)methyl]-5-methyl-2-{2-[(4-phenoxyphenyl)-formamido]acetyl}-2-azabicyclo[3.1.0]hexane-3-carboxamide (Compound 5). TFA (1 mL) was added to a solution of compound B (23 mg, 0.04 mmol, 1.0 equiv.) in CH₂Cl₂ (1 mL). The reaction was stirred at room temperature for 4 h and then concentrated to dryness. 3 M HCl (2 mL) was added and the reaction mixture was concentrated to dryness. The residue was purified directly on HPLC to give Compound 5 (10 mg, 0.02 mmol, Yield 52.19%) as a white solid. ¹H NMR (400 MHz, DMSO-d₆) δ 8.96-8.84 (m, 1H), 8.75 (t, J=5.7 Hz, 1H), 8.67-8.53 (m, 1H), 8.42 (s, 2H), 8.19-8.05 (m, 1H), 7.89 (dd, J=8.2, 5.9 Hz, 2H), 7.54-7.40 (m, 3H), 7.22 (t, J=7.4 Hz, 1H), 7.06 (dd, J=27.4, 8.2 Hz, 2H), 4.75 (dd, J=11.5, 3.1 Hz, 1H), 4.52-4.32 (m, 3H), 4.04 (dd, J=16.4, 5.4 Hz, 1H), 2.39-2.28 (m, 1H), 2.10-1.99 (m, 1H), 1.23 (d, J=11.7 Hz, 4H), 0.69 (t, J=5.5 Hz, 1H). LC/MS (ESI) m/z: 500 (M+H)⁺.

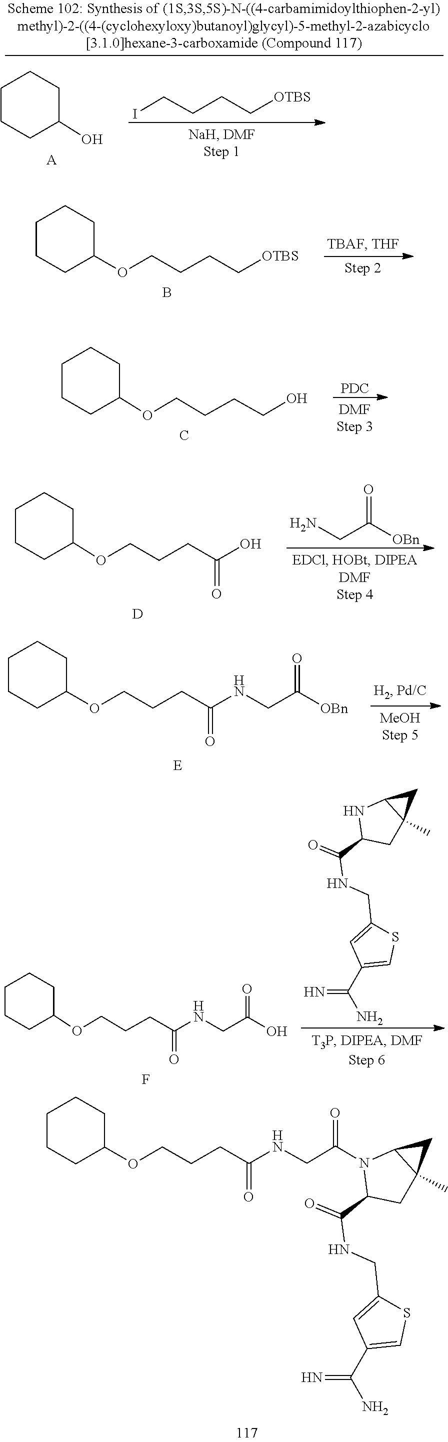

Step 1: tert-butyl(4-(cyclohexyloxy)butoxy)dimethylsilane (B). To a solution of compound A (1 g, 10.0 mmol) in DMF (10 mL) was added NaH (800 mg, 20.0 mmol, 60% dispersion in mineral oil) at 0° C. under N₂ atmosphere and the mixture was stirred at 0° C. for 30 min. A solution of tert-butyl(4-iodobutoxy)dimethylsilane (3.14 g, 10.0 mmol) in DMF (2 mL) was added to the mixture at 0° C. and the mixture was stirred under N₂ atmosphere at 25° C. for 2 hours. The reaction was quenched with saturated aq.NH₄Cl solution and extracted with MTBE twice. The combined organic layers were washed with brine, dried over anhydrous Na₂SO₄, filtered and concentrated to dryness under reduced pressure. The residue was purified by flash chromatography on silica gel (PE:EtOAc=100:1) to give compound B (600 mg, yield 21.0%) as a colorless oil.

Step 2: 4-(cyclohexyloxy)butan-1-ol (C). To a solution of compound B (600 mg, 2.10 mmol) in THE (6 mL) was added TBAF (4.2 mL, 4.20 mmol, 1 M in THF) 0° C. and the reaction mixture was stirred at room temperature for 2 hours. The mixture was diluted with EtOAc and washed with saturated aq.NH₄Cl solution. The aqueous layer was extracted with EtOAc twice and the combined organic layers were washed with brine, dried over anhydrous Na₂SO₄, filtered and concentrated to dryness under reduced pressure. The residue was purified by flash chromatography on silica gel (PE:EtOAc=10:1 to 5:1) to give compound C (220 mg, yield 58.1%) as colorless oil.

Step 3: 4-(cyclohexyloxy)butanoic acid (D). To a solution of compound C (220 mg, 1.22 mmol) in DMF (2 mL) was added PDC (1.37 g, 3.66 mmol) at 0° C. and the reaction was stirred at room temperature for 16 hours. The mixture was diluted with EtOAc and washed with saturated aq. NH₄Cl solution. The aqueous layer was extracted with EtOAc twice and the combined organic layers were washed with brine, dried over anhydrous Na₂SO₄, filtered and concentrated to dryness under reduced pressure. The residue was purified by flash chromatography on silica gel (PE:EtOAc=5:1 to 2:1) to give compound D (220 mg, yield 92.2%) as a colorless oil. LC/MS (ESI) m/z: 185 (M−H)⁻.

Step 4: benzyl (4-(cyclohexyloxy)butanoyl)glycinate (E). To a mixture of compound D (220 mg, 1.18 mmol) and benzyl glycinate hydrochloride (238 mg, 1.18 mmol) in DMF (2 mL) was added DIPEA (0.97 mL, 5.90 mmol), HOBt (319 mg, 2.36 mmol) and EDCI (452 mg, 2.36 mmol) at 0° C. under N₂ atmosphere and the reaction mixture was stirred under N₂ atmosphere at room temperature for 2 hours. The mixture was diluted with EtOAc and washed with saturated aq. NH₄Cl solution. The aqueous layer was extracted with EtOAc twice and the combined organic layers were washed with brine, dried over anhydrous Na₂SO₄, filtered and concentrated to dryness under reduced pressure. The residue was purified by flash chromatography on silica gel (PE:EtOAc=10:1 to 5:1) to give compound E (155 mg, yield 39.4%) as a yellow oil. LC/MS (ESI) m/z: 334 (M+H)⁺.

Step 5: (4-(cyclohexyloxy)butanoyl)glycine (F). To a solution of compound E (150 mg, 0.46 mmol) in MeOH (3 mL) was added Pd/C (50 mg, 10% wt) at room temperature under N₂ atmosphere and the reaction mixture was stirred under a H₂ balloon at room temperature for 2 hours. The mixture was filtered and the filtrate was concentrated to dryness under reduced pressure to give compound F (100 mg, yield 89.5%) as a colorless oil, which was used directly in the next step. LC/MS (ESI) m/z: 244 (M+H)⁺.

Step 6: tert-butyl ((5-(((2S,4R)-4-cyclopropoxy-1-((4-phenoxybenzoyl)glycyl)pyrrolidine-2-carboxamido)methyl)thiophen-3-yl)(imino)methyl)carbamate (Compound 117). To a mixture of compound F (100 mg, 0.41 mmol) and (1 S,3S,5S)-N-((4-carbamimidoylthiophen-2-yl)methyl)-5-methyl-2-azabicyclo[3.1.0]hexane-3-carboxamide (114 mg, 0.41 mmol) in DMF (2 mL) was added DIPEA (0.41 mL, 2.46 mmol) and T₃P (782 mg, 1.23 mmol, 50% wt in EtOAc) at 0° C. under N₂ atmosphere and the reaction mixture was stirred under N₂ atmosphere at room temperature for 16 hours. The mixture was diluted with EtOAc and washed with saturated aq.NaHCO₃ solution. The aqueous layer was extracted with CHCl₃/i-PrOH (v/v=3/1) three times and the combined organic layers were dried over anhydrous Na₂SO₄, filtered and concentrated to dryness under reduced pressure. The residue was purified by prep-HPLC to give Compound 117 (8.9 mg, yield 4.31%) as a yellow oil. ¹H NMR (400 MHz, CD₃OD) δ 8.24 (d, J=1.6 Hz, 1H), 7.42 (d, J=0.8 Hz, 1H), 4.82-4.78 (m, 1H), 4.58-4.48 (m, 2H), 4.22-4.09 (m, 2H), 3.49 (t, J=6.4 Hz, 2H), 3.33-3.30 (m, 2H), 2.39-2.33 (m, 3H), 2.20-2.17 (m, 1H), 1.90-1.83 (m, 5H), 1.75-1.72 (m, 2H), 1.60-1.50 (m, 1H), 1.28-1.24 (m, 10H), 1.16-1.08 (m, 1H), 0.82-0.80 (m, 1H); LC/MS (ESI) m/z: 504 (M+H)⁺.

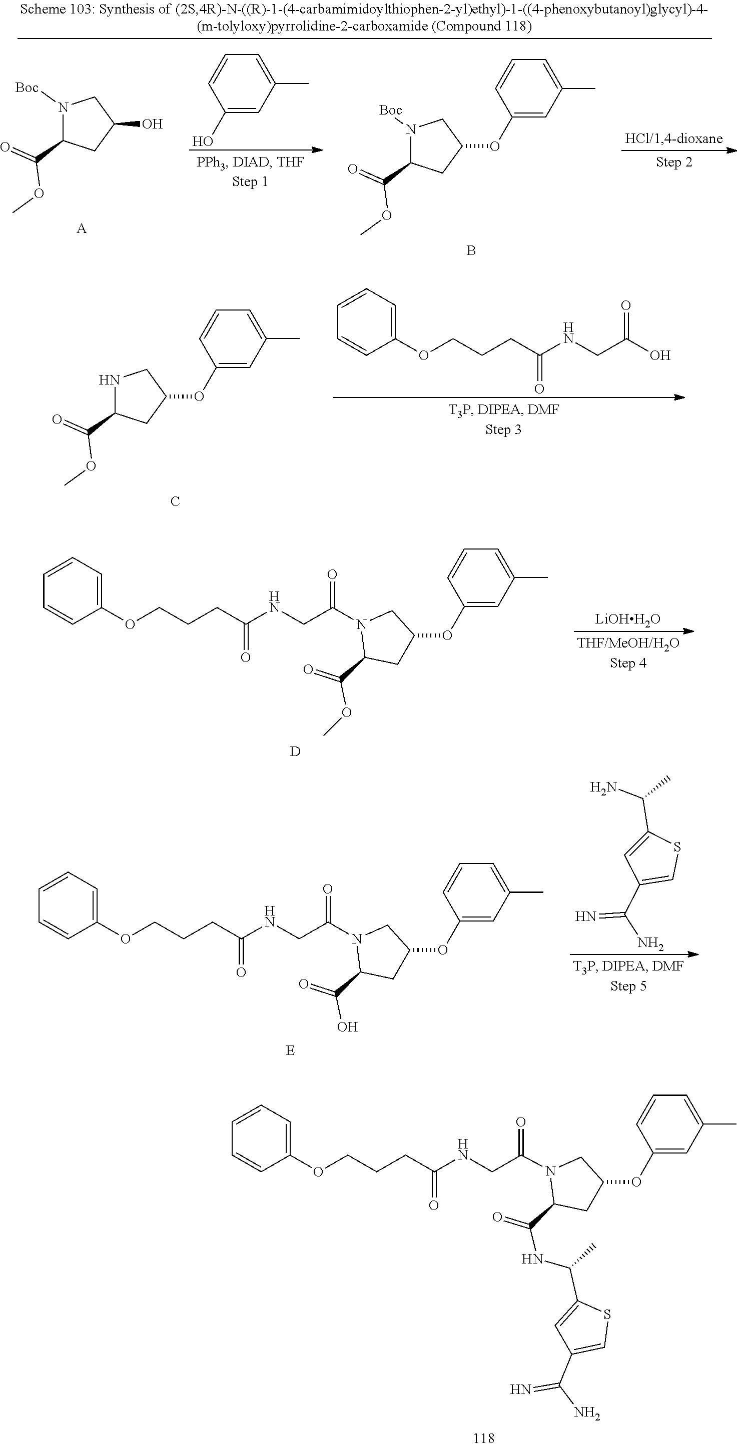

Step 1: 1-(tert-butyl) 2-methyl (2S,4R)-4-(m-tolyloxy)pyrrolidine-1,2-dicarboxylate (B). To a mixture of compound A (1 g, 4.08 mmol) and m-cresol (661 mg, 6.12 mmol) in THF (20 mL) was added PPh₃ (1.6 g, 6.12 mmol) and DIAD (1.3 g, 6.12 mmol) at 0° C. under N₂ atmosphere and the reaction mixture was stirred under N₂ atmosphere at room temperature for 16 hours. The mixture was diluted with water and extracted with EtOAc twice. The combined organic layers were washed with brine, dried over anhydrous Na₂SO₄, filtered and concentrated to dryness under reduced pressure. The residue was purified by flash chromatography on silica gel (PE:EtOAc=10:1) to give compound B (550 mg, yield 40.2%) as a white solid. LC/MS (ESI) m/z: 336 (M+H)⁺.

Step 2: methyl (2S,4R)-4-(m-tolyloxy)pyrrolidine-2-carboxylate (C). To a solution of compound B (550 mg, 1.64 mmol) in 1,4-dioxane (3 mL) was added HCl/1,4-dioxane (3 mL) at 0° C. and the reaction mixture was stirred at room temperature for 2 hours. The mixture was concentrated to dryness under reduced pressure, co-evaporated with DCM twice and dried under vacuum to give compound C (380 mg, yield 98.5%) as a white solid, which was used directly in the next step. LC/MS (ESI) m/z: 236 (M+H)⁺.

Step 3: methyl (2S,4R)-1-((4-phenoxybutanoyl)glycyl)-4-(m-tolyloxy)pyrrolidine-2-carboxylate (D). To a mixture of compound C (380 mg, 1.62 mmol) and (4-phenoxybutanoyl)glycine (383 mg, 1.62 mmol) in DMF (10 mL) was added DIPEA (1.3 g, 9.69 mmol) and T₃P (3.1 g, 4.85 mmol, 50% wt in EtOAc) at 0° C. under N₂ atmosphere and the reaction mixture was stirred under N₂ atmosphere at room temperature for 16 hours. The mixture was diluted with EtOAc and washed with saturated aq.NaHCO₃ solution. The aqueous layer was extracted with EtOAc twice and the combined organic layers were washed with brine, dried over anhydrous Na₂SO₄, filtered and concentrated to dryness under reduced pressure. The residue was purified by flash chromatography on silica gel (PE:EtOAc=1:1) to give compound D (470 mg, yield 64.0%) as a white solid. LC/MS (ESI) m/z: 455 (M+H)⁺.

Step 4: (2S,4R)-1-((4-phenoxybutanoyl)glycyl)-4-(m-tolyloxy)pyrrolidine-2-carboxylic acid (E). To a solution of compound D (470 mg, 1.03 mmol) in THE (4 mL), MeOH (1 mL) and H₂O (1 mL) was added LiOH·H₂O (43 mg, 1.03 mmol) at 0° C. and the reaction mixture was stirred at room temperature for 3 hours. The mixture was concentrated to dryness in vacuo and washed with EtOAc. The aqueous layer was acidified with 1 N aq. HCl solution to pH˜3 and extracted with EtOAc three times. The combined organic layers were dried over anhydrous Na₂SO₄, filtered and concentrated to dryness under reduced pressure to give compound E (430 mg, yield 94.4%) as a colorless oil, which was used directly in the next step. LC/MS (ESI) m/z: 441 (M+H)⁺.

Step 5: (2S,4R)-N-((R)-1-(4-carbamimidoylthiophen-2-yl)ethyl)-1-((4-phenoxybutanoyl)-glycyl)-4-(m-tolyloxy)pyrrolidine-2-carboxamide (Compound 118). To a mixture of compound E (100 mg, 0.23 mmol) and (R)-5-(1-aminoethyl)thiophene-3-carboximidamide (38 mg, 0.23 mmol) in DMF (5 mL) was added DIPEA (176 mg, 1.36 mmol) and T₃P (433 mg, 0.68 mmol, 50% wt in EtOAc) at 0° C. under N₂ atmosphere and the reaction mixture was stirred under N₂ atmosphere at room temperature for 16 hours. The mixture was diluted with EtOAc and washed with saturated aq. NaHCO₃ solution. The aqueous layer was extracted with CHCl₃/i-PrOH (v/v=3/1) three times and the combined organic layers were dried over anhydrous Na₂SO₄, filtered and concentrated to dryness under reduced pressure. The residue was purified by prep-HPLC to give Compound 118 (11 mg, yield 8.1%) as a white solid. ¹H NMR (400 MHz, CD₃OD) δ 8.55 (d, J=2.9 Hz, 1H), 8.23 (d, J=1.3 Hz, 1H), 7.57 (s, 1H), 7.25-7.15 (m, 3H), 6.91-6.87 (m, 3H), 6.82-6.71 (m, 3H), 5.38-5.22 (m, 1H), 5.08-5.04 (m, 1H), 4.59-4.55 (m, 1H), 4.07-3.85 (m, 6H), 2.55-2.42 (m, 3H), 2.30 (s, 3H), 2.24-2.17 (m, 1H), 2.10-2.03 (m, 2H), 1.57 (d, J=7.0 Hz, 3H); LC/MS (ESI) m/z: 592 (M+H)⁺.

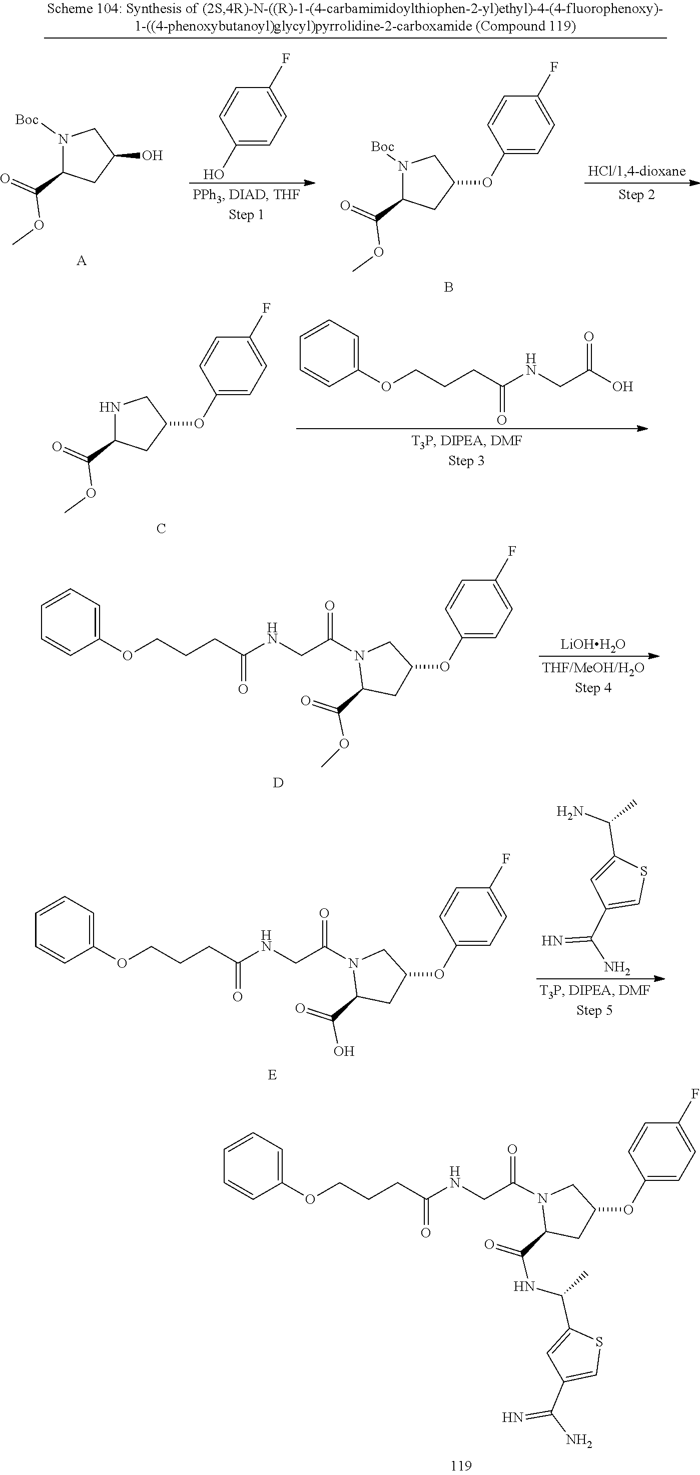

Step 1: 1-(tert-butyl) 2-methyl (2S,4R)-4-(4-fluorophenoxy)pyrrolidine-1,2-dicarboxylate (B). To a mixture of compound A (1 g, 4.08 mmol) and 4-fluorophenol (686 mg, 6.12 mmol) in THE (20 mL) was added PPh₃ (1.6 g, 6.12 mmol) and DIAD (1.2 g, 6.12 mmol) at 0° C. under N₂ atmosphere and the reaction mixture was stirred under N₂ atmosphere at room temperature for 16 hours. The mixture was diluted with water and extracted with EtOAc twice. The combined organic layers were washed with brine, dried over anhydrous Na₂SO₄, filtered and concentrated to dryness under reduced pressure. The residue was purified by flash chromatography on silica gel (PE:EtOAc=10:1) to give compound B (680 mg, yield 49.1%) as a white solid. LC/MS (ESI) m/z: 340 (M+H)⁺.

Step 2: methyl (2S,4R)-4-(4-fluorophenoxy)pyrrolidine-2-carboxylate (C). To a solution of compound B (680 mg, 2.0 mmol) in 1,4-dioxane (3 mL) was added HCl/1,4-dioxane (3 mL) at 0° C. and the reaction mixture was stirred at room temperature for 2 hours. The mixture was concentrated to dryness under reduced pressure, co-evaporated with DCM twice and dried under vacuum to give compound 3 (470 mg, yield 98.0%) as a white solid, which was used directly in the next step. LC/MS (ESI) m/z: 240 (M+H)⁺.

Step 3: methyl (2S,4R)-4-(4-fluorophenoxy)-1-((4-phenoxybutanoyl)glycyl)pyrrolidine-2-carboxylate (D). To a mixture of compound C (470 mg, 1.97 mmol) and (4-phenoxybutanoyl)glycine (466 mg, 1.97 mmol) in DMF (10 mL) was added DIPEA (1.5 g, 11.79 mmol) and T₃P (3.8 g, 5.89 mmol, 50% wt in EtOAc) at 0° C. under N₂ atmosphere and the reaction mixture was stirred under N₂ atmosphere at room temperature for 16 hours. The mixture was diluted with EtOAc and washed with saturated aq.NaHCO₃ solution. The aqueous layer was extracted with EtOAc twice and the combined organic layers were washed with brine, dried over anhydrous Na₂SO₄, filtered and concentrated to dryness under reduced pressure. The residue was purified by flash chromatography on silica gel (PE:EtOAc=1:2) to give compound D (590 mg, yield 65.5%) as a white solid. LC/MS (ESI) m/z: 459 (M+H)⁺.

Step 4: (2S,4R)-4-(4-fluorophenoxy)-1-((4-phenoxybutanoyl)glycyl)pyrrolidine-2-carboxylic acid (E). To a solution of compound D (590 mg, 1.29 mmol) in THE (4 mL), MeOH (1 mL) and H₂O (1 mL) was added LiOH·H₂O (54 mg, 1.29 mmol) at 0° C. and the reaction mixture was stirred at room temperature for 3 hours. The mixture was diluted with EtOAc and washed with 1 N aq. HCl solution. The mixture was concentrated to dryness in vacuo and washed with EtOAc. The aqueous layer was acidified with 1 N aq.HCl solution to pH-3 and extracted with EtOAc three times. The combined organic layers were dried over anhydrous Na₂SO₄, filtered and concentrated to dryness under reduced pressure to give compound E (560 mg, 1.26 mmol, yield 97.9%) as a colorless oil, which was used directly in the next step. LC/MS (ESI) m/z: 445 (M+H)⁺.

Step 5: (2S,4R)-N-((R)-1-(4-carbamimidoylthiophen-2-yl)ethyl)-4-(4-fluorophenoxy)-1-((4-phenoxybutanoyl)glycyl)pyrrolidine-2-carboxamide (Compound 119). To a mixture of compound E (100 mg, 0.23 mmol) and (R)-5-(1-aminoethyl)thiophene-3-carboximidamide (38 mg, 0.23 mmol) in DMF (5 mL) was added DIPEA (174 mg, 1.35 mmol) and T₃P (430 mg, 0.68 mmol, 50% wt in EtOAc) at 0° C. under N₂ atmosphere and the reaction mixture was stirred under N₂ atmosphere at room temperature for 16 hours. The mixture was diluted with EtOAc and washed with saturated aq. NaHCO₃ solution. The aqueous layer was extracted with CHCl₃/i-PrOH (v/v=3/1) three times and the combined organic layers were dried over anhydrous Na₂SO₄, filtered and concentrated to dryness under reduced pressure. The residue was purified by prep-HPLC to give Compound 119 (11 mg, yield 8.02%) as a white solid. ¹H NMR (400 MHz, CD₃OD) δ 8.23 (s, 1H), 7.67 (s, 1H), 7.25-7.20 (m, 2H), 7.07-7.01 (m, 2H), 6.97-6.93 (m, 2H), 6.91-6.87 (m, 3H), 5.35-5.23 (m, 1H), 5.05-5.00 (m, 1H), 4.66-4.55 (m, 1H), 4.09-3.87 (m, 6H), 2.56-2.44 (m, 3H), 2.24-2.17 (m, 1H), 2.10-2.04 (m, 2H), 1.57 (d, J=7.0 Hz, 3H); LC/MS (ESI) m/z: 596 (M+H)⁺.

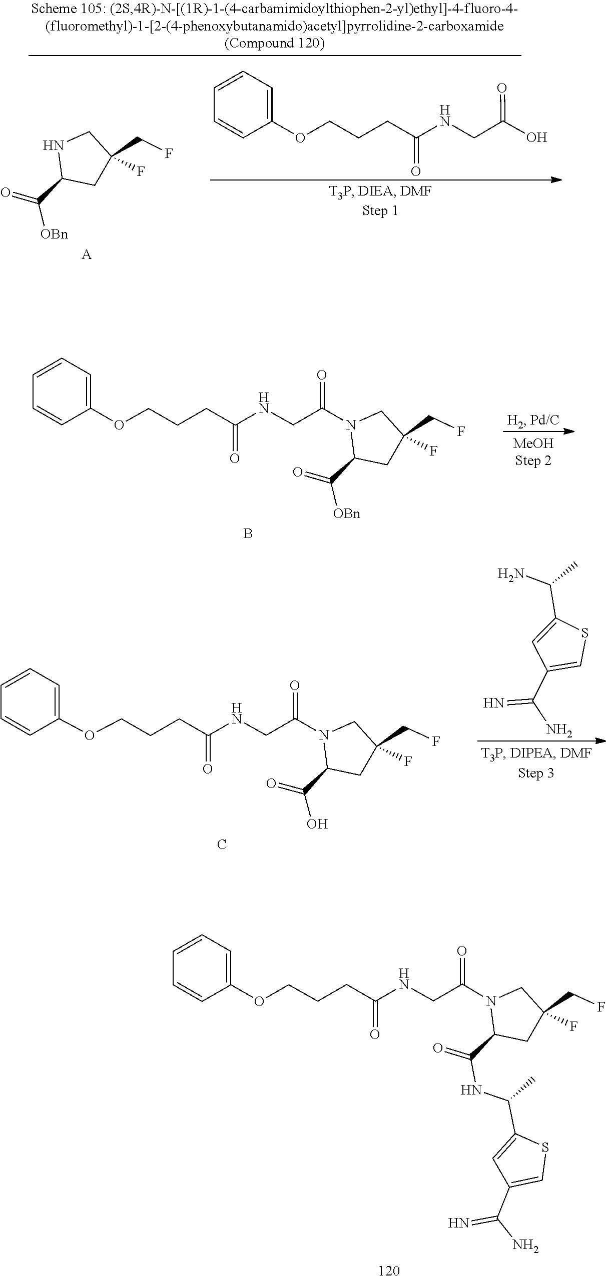

Step 1: benzyl (2S,4R)-4-fluoro-4-(fluoromethyl)-1-((4-phenoxybutanoyl)glycyl)pyrrolidine-2-carboxylate (B). To a mixture of compound A (172 mg, 0.67 mmol) and 4-oxo-7-phenoxyheptanoic acid (159 mg, 0.67 mmol) in DMF (6 mL) was added DIPEA (0.71 mL, 4.04 mmol) and T₃P (1.29 g, 2.02 mmol, 50% wt in EtOAc) at 0° C. under N₂ atmosphere and the mixture was stirred under N₂ atmosphere at 25° C. for 2 hours. The mixture was diluted with EtOAc and washed with saturated aq.NaHCO₃ solution. The aqueous layer was extracted with EtOAc twice and the combined organic layers were washed with brine, dried over anhydrous Na₂SO₄, filtered and concentrated to dryness under reduced pressure. The residue was purified by flash chromatography on silica gel (DCM:MeOH=15:1) to give compound B (320 mg, 99.9% yield) as a light-yellow oil. LC/MS (ESI) (m/z): 475 (M+H)⁺.

Step 2: (2S,4R)-4-fluoro-4-(fluoromethyl)-1-[2-(4-phenoxybutanamido)acetyl]pyrrolidine-2-carboxylic acid (C). To a solution of compound B (320 mg, 0.67 mmol) in MeOH (7 mL) was added Pd/C (100 mg, 10% wt) at room temperature under N₂ atmosphere and the mixture was stirred under a H₂ balloon at room temperature for 2 hours. The mixture was filtered and the filtrate was concentrated to dryness under reduced pressure to give compound C (190 mg, 73.3% yield) as a light-yellow oil, which was used directly in the next step. LC/MS (ESI) (m/z): 385 (M+H)⁺.

Step 3: (2S,4R)-N-[(1R)-1-(4-carbamimidoylthiophen-2-yl)ethyl]-4-fluoro-4-(fluoromethyl)-1-[2-(4-phenoxybutanamido)acetyl]pyrrolidine-2-carboxamide (Compound 120). To a mixture of compound C (80 mg, 0.21 mmol) and 5-[(1R)-1-aminoethyl]thiophene-3-carboximidamide (53 mg, 0.31 mmol) in DMF (3 mL) was added DIPEA (0.22 mL, 1.25 mmol) and T₃P (397 mg, 0.62 mmol, 50% wt in EtOAc) at 0° C. under N₂ atmosphere and the mixture was stirred under N₂ atmosphere at 25° C. for 1.5 hours. The mixture was diluted with EtOAc and washed with saturated aq. NaHCO₃ solution. The aqueous layer was extracted with CHCl₃/i-PrOH (v/v=3/1) three times. The combined organic layers were dried over anhydrous Na₂SO₄, filtered and concentrated to dryness under reduced pressure. The residue was purified by prep-HPLC to give Compound 120 (11.4 mg, 10.2% yield) as a white solid. ¹H NMR (400 MHz, CD₃OD) δ 8.23 (d, J=1.6 Hz, 1H), 7.56 (s, 1H), 7.27-7.20 (m, 2H), 6.93-6.86 (m, 3H), 5.35-5.22 (m, 1H), 4.75-4.69 (m, 1H), 4.64-4.59 (m, 2H), 4.14-3.84 (m, 6H), 2.49-2.43 (m, 3H), 2.25-2.04 (m, 3H), 1.58 (d, J=7.2 Hz, 3H). LC/MS (ESI) (m/z): 536 (M+H)⁺.

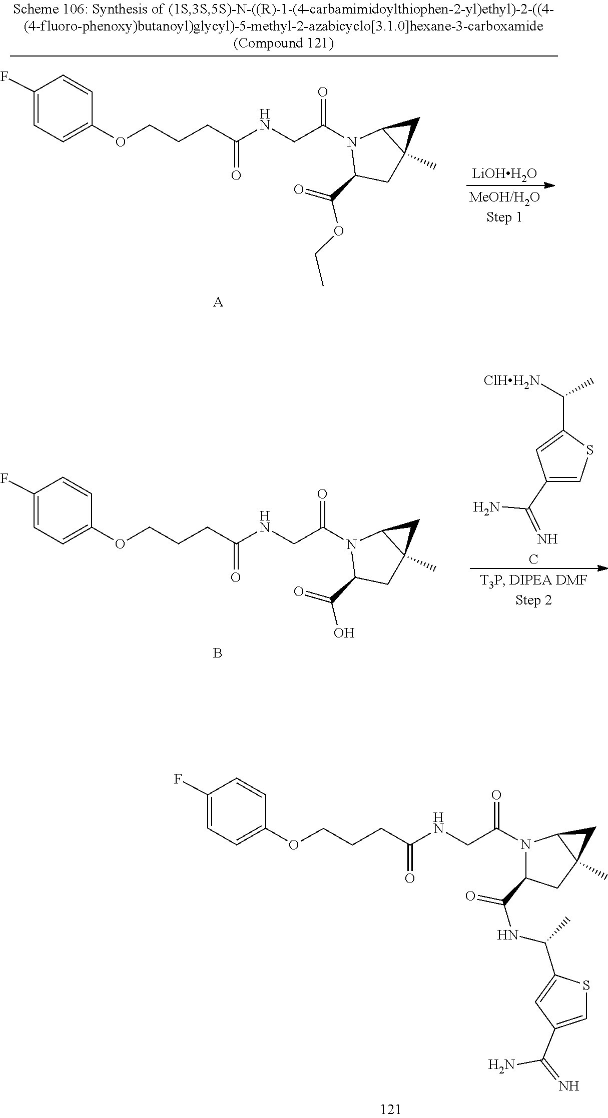

Step 1: (1S,3S,5S)-2-((4-(4-fluorophenoxy)butanoyl)glycyl)-5-methyl-2-azabicyclo[3.1.0]hexane-3-carboxylic acid (B). To a mixture of ethyl (1S,3S,5S)-2-((4-(4-fluorophenoxy)butanoyl)glycyl)-5-methyl-2-azabicyclo[3.1.0]hexane-3-carboxylate (A; 140 mg, 0.34 mmol) in methanol (1.5 mL) and water (0.5 mL) was added a solution of LiOH·H₂O (15 mg, 0.34 mmol) at 0° C. and the mixture was stirred at room temperature for 16 hours. The mixture was acidified with 1 N aq. HCl solution to pH<3 and extracted with ethyl acetate (2×10 mL). The combined organic layers were concentrated to dryness under reduced pressure to give (1S,3S,5S)-2-((4-(4-fluorophenoxy)butanoyl)glycyl)-5-methyl-2-azabicyclo[3.1.0]hexane-3-carboxylic acid (B; 120 mg, 92.1% yield) as a yellow oil, which was used directly in next step without further purification. LC/MS (ESI) (m/z): 379(M+H)⁺.

Step 2: (1S,3S,5S)-N-((R)-1-(4-carbamimidoylthiophen-2-yl)ethyl)-2-((4-(4-fluorophenoxy) butanoyl)glycyl)-5-methyl-2-azabicyclo[3.1.0]hexane-3-carboxamide (Compound 121). To a mixture of (1S,3S,5S)-2-((4-(4-fluorophenoxy)butanoyl)glycyl)-5-methyl-2-azabicyclo-[3.1.0]hexane-3-carboxylic acid (100 mg, 0.264 mmol) and (R)-5-(1-aminoethyl)thiophene-3-carboximidamide hydrochloride (C; 81.5 mg, 0.396 mmol) in DMF (3 mL) was added HATU (120.5 mg, 0.317 mmol) and DIPEA (136.4 mg, 1.06 mmol). The mixture was stirred at room temperature under N₂ atmosphere for 1 hour. The reaction was quenched with saturated aq. NH₄Cl solution. The resulting mixture was extracted with ethyl acetate (3×30 mL). The combined organic layers were washed with water (2×30 mL) and brine (2×30 mL), dried over anhydrous Na₂SO₄, filtered and concentrated to dryness under reduced pressure. The residue was purified by prep-HPLC to give Compound 121(41 mg, 28.6% yield) as a white solid. ¹H NMR (400 MHz, CD₃OD) δ 8.21 (d, J=1.4 Hz, 1H), 7.49 (s, 1H), 6.98-6.93 (m, 2H), 6.89-6.85 (m, 2H), 5.19 (dd, J=12.6, 6.0 Hz, 1H), 4.79 (dd, J=11.4, 3.5 Hz, 1H), 4.16 (dd, J=63.5, 16.6 Hz, 2H), 3.97 (t, J=6.2 Hz, 2H), 2.45 (t, J=7.4 Hz, 2H), 2.40 (t, J=9.5 Hz, 1H), 2.14 (dd, J=13.3, 3.5 Hz, 1H), 2.08-2.03 (m, 2H), 1.58 (dd, J=21.0, 7.0 Hz, 3H), 1.28 (s, 3H), 1.19 (dd, J=5.5, 2.3 Hz, 1H), 0.78 (t, J=5.2 Hz, 1H); LC/MS(ESI) m/z: 530 (M+H)⁺.

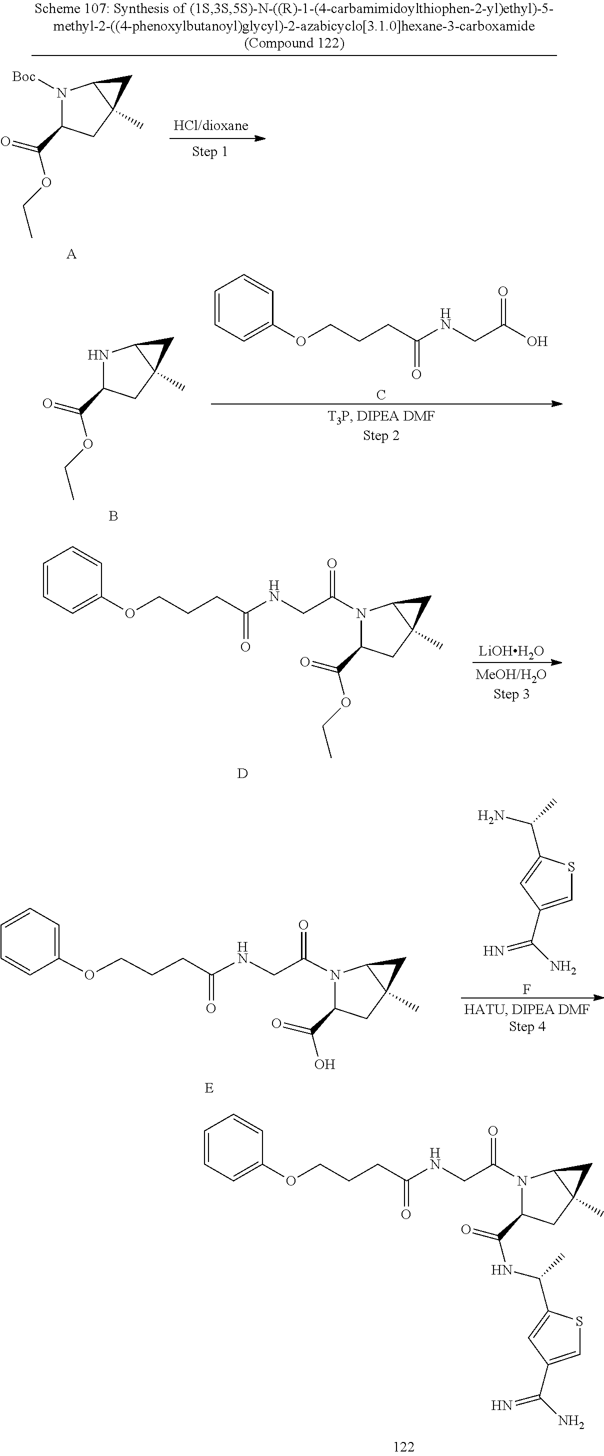

Step 1: ethyl (1S,3S,5S)-5-methyl-2-azabicyclo[3.1.0]hexane-3-carboxylate (B). A mixture of 2-(tert-butyl) 3-ethyl (1 S,3S,5S)-5-methyl-2-azabicyclo[3.1.0]hexane-2,3-dicarboxylate (A; 350 mg, 1.3 mmol) and HCl/1,4-dioxane (4 mL) was stirred at room temperature for 3 hours. The reaction mixture was concentrated to dryness under reduced pressure, diluted with DCM again and dried under vacuum to give ethyl (1 S,3S,5S)-5-methyl-2-azabicyclo[3.1.0]hexane-3-carboxylate (B; 175 mg, yield 79.9%), which was used directly in the next step without further purification. LC/MS (ESI) m/z: 170 (M+H)⁺.

Step 2: ethyl (1 S,3S,5S)-5-methyl-2-((4-phenoxybutanoyl)glycyl)-2-azabicyclo[3.1.0]hexane-3-carboxylate (C). To a mixture of (4-phenoxybutanoyl)glycine (C; 205 mg, 0.86 mmol) and ethyl (1S,3S,5S)-5-methyl-2-azabicyclo[3.1.0]hexane-3-carboxylate (B; 175 mg, 1.03 mmol) in DMF (3 mL) was added DIPEA (668 mg, 5.17 mmol) and T₃P (1.6 g, 2.59 mmol, 50% in ethyl acetate) at 0° C. under N₂ atmosphere and the mixture was stirred at 35° C. for 4 hours. The mixture was quenched with saturated aq.NaHCO₃ solution and extracted with ethyl acetate twice. The combined organic layers were washed with water and brine, dried over anhydrous Na₂SO₄, filtered and concentrated to dryness under reduced pressure. The residue was purified by prep-HPLC to give ethyl (1 S,3S,5S)-5-methyl-2-((4-phenoxybutanoyl)glycyl)-2-azabicyclo[3.1.0]hexane-3-carboxylate (D; 300 mg, 90.1% yield) as an oil. LC/MS (ESI) (m/z): 389 (M+H)+.

Step 3: (1S,3S,5S)-5-methyl-2-((4-phenoxybutanoyl)glycyl)-2-azabicyclo[3.1.0]hexane-3-carboxylic acid (E). To a solution of ethyl (1S,3S,5S)-5-methyl-2-((4-phenoxybutanoyl)glycyl)-2-azabicyclo[3.1.0]hexane-3-carboxylate (D; 300 mg, 0.77 mmol) in MeOH (3 mL) and water (1 mL) was added LiOH·H₂O (107 g, 2.55 mmol) at 0° C. and the mixture was stirred at room temperature for 3 hours. The mixture was acidified with 1 N aq. HCl solution to pH=3 and concentrated to dryness under reduced pressure to give (1 S,3S,5S)-5-methyl-2-((4-phenoxybutanoyl)glycyl)-2-azabicyclo[3.1.0]hexane-3-carboxylic acid (E; 270 mg, 97.1% yield) as a light yellow solid, which was used directly in next step without further purification. LC/MS (ESI) (m/z): 361 (M+H)⁺.

Step 4: (1S,3S,5S)-N-((R)-1-(4-carbamimidoylthiophen-2-yl)ethyl)-5-methyl-2-((4-phenoxy-butanoyl)glycyl)-2-azabicyclo[3.1.0]hexane-3-carboxamide (Compound 122). To a mixture of (1 S,3S,5S)-5-methyl-2-((4-phenoxybutanoyl)glycyl)-2-azabicyclo[3.1.0]hexane-3-carboxylic acid (100 mg, 0.28 mmol) and (R)-5-(1-aminoethyl)thiophene-3-carboximidamide (86 mg, 0.42 mmol) in DMF (2 mL) was added DIPEA (180 mg, 1.4 mmol) and HATU (129 mg, 0.34 mmol) at 0° C. under N₂ atmosphere and the mixture was stirred at 35° C. for 4 hours. The mixture was quenched with saturated aq. NaHCO₃ solution and extracted with ethyl acetate twice. The combined organic layers were washed with water and brine, dried over anhydrous Na₂SO₄, filtered and concentrated to dryness. The residue was purified by prep-HPLC to give Compound 122 (9.4 mg, 6.6% yield) as a white solid. ¹H NMR (400 MHz, CD₃OD) δ 8.55 (s, 1H), 8.21 (d, J=1.6 Hz, 1H), 7.49 (d, J=1.4 Hz, 1H), 7.23 (s, 2H), 6.90-6.87 (m, 3H), 5.20 (d, J=6.9 Hz, 1H), 4.78 (d, J=3.5 Hz, 1H), 4.57 (s, 2H), 4.24 (d, J=16.7 Hz, 1H), 4.08 (d, J=16.6 Hz, 1H), 3.99 (d, J=6.3 Hz, 2H), 2.44 (dt, J=24.5, 9.5 Hz, 4H), 2.17-2.13 (m, 1H), 2.11-2.03 (m, 3H), 1.56 (d, J=7.0 Hz, 3H), 1.20-1.18 (m, 1H), 0.78 (t, J=5.4 Hz, 1H); LC/MS (ESI) (m/z): 512 (M+H)⁺.

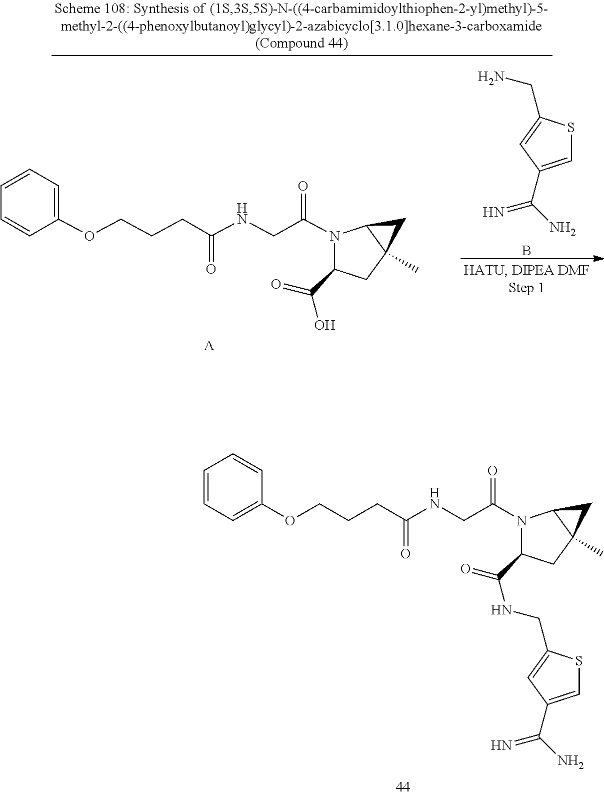

Step 1: (1S,3S,5S)-N-((4-carbamimidoylthiophen-2-yl)methyl)-5-methyl-2-((4-phenoxy-butanoyl)glycyl)-2-azabicyclo[3.1.0]hexane-3-carboxamide (Compound 44). To a mixture of (1S,3S,5S)-5-methyl-2-((4-phenoxybutanoyl)glycyl)-2-azabicyclo[3.1.0]hexane-3-carboxylic acid (A; 94 mg, 0.26 mmol) and 5-(aminomethyl)thiophene-3-carboximidamide (B; 75 mg, 0.39 mmol) in DMF (2 mL) was added DIPEA (202 mg, 1.57 mmol) and HATU (119 mg, 0.31 mmol) at 0° C. under N₂ atmosphere and the mixture was stirred at 35° C. for 4 hours. The mixture was quenched with saturated aq. NaHCO₃ solution and extracted with ethyl acetate twice. The combined organic layers were washed with water and brine, dried over anhydrous Na₂SO₄, filtered and concentrated to dryness under reduced pressure. The residue was purified by prep-HPLC to give (1 S,3S,5S)-N-((4-carbamimidoylthiophen-2-yl)methyl)-5-methyl-2-((4-phenoxybutanoyl) glycyl)-2-azabicyclo[3.1.0]hexane-3-carboxamide (Compound 44; 16 mg, 12.4% yield) as white solid. ¹H NMR (400 MHz, CD₃OD) δ 8.22 (d, J=1.5 Hz, 1H), 7.41 (s, 1H), 7.23 (d, J=8.2 Hz, 2H), 6.90 (dd, J=6.1, 3.4 Hz, 3H), 4.82-4.78 (m, 1H), 4.52 (d, J=10.7 Hz, 2H), 4.17 (d, J=15.9 Hz, 2H), 4.00 (s, 2H), 3.35-3.32 (m, 1H), 2.46 (d, J=7.6 Hz, 2H), 2.37 (d, J=13.0 Hz, 1H), 2.16 (dd, J=13.4, 3.3 Hz, 1H), 2.09-2.04 (m, 2H), 1.28 (s, 3H), 1.12 (dd, J=5.7, 2.4 Hz, 1H), 0.80 (s, 1H); LC/MS (ESI) (m/z): 498 (M+H)⁺.

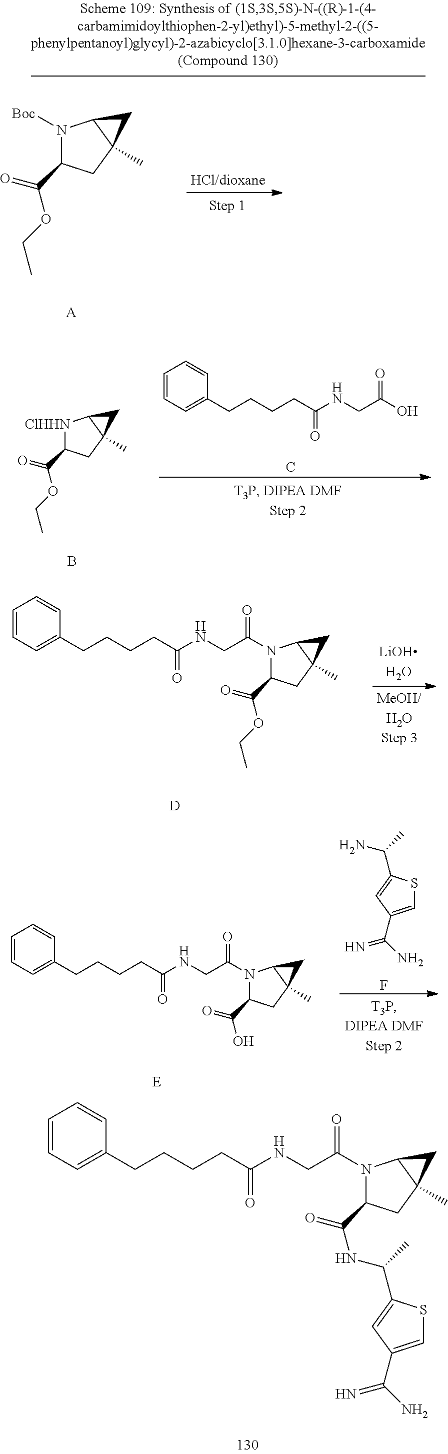

Step 1: ethyl (1S,3S,5S)-5-methyl-2-azabicyclo[3.1.0]hexane-3-carboxylate hydrochloride (B). A solution of 2-(tert-butyl) 3-ethyl (1 S,3S,5S)-5-methyl-2-azabicyclo[3.1.0]hexane-2,3-dicarboxylate (A; 100 mg, 0.39 mmol) in HCl/1,4-dioxane (1 mL) was stirred at 25° C. for 1 hour. The mixture was concentrated to dryness to give ethyl (1 S,3S,5S)-5-methyl-2-azabicyclo[3.1.0]hexane-3-carboxylate hydrochloride (B; 66 mg, 99.4% yield) as a white solid, which was used directly without purification. LC/MS (ESI) m/z: 170(M+H)⁺.

Step 2: ethyl (1S,3S,5S)-5-methyl-2-((5-phenylpentanoyl)glycyl)-2-azabicyclo[3.1.0]hexane-3-carboxylate (D). To a solution of ethyl (1 S,3S,5S)-5-methyl-2-azabicyclo[3.1.0]hexane-3-carboxylate hydrochloride (B; 66 mg, 0.39 mmol) and (C; 5-phenylpentanoyl)glycine (110 mg, 0.47 mmol) in DMF (1 mL) was added DIPEA (403 mg, 3.12 mmol) and T₃P (372 mg, 1.17 mmol, 50% in ethyl acetate). The mixture was stirred at room temperature under N₂ atmosphere overnight. The reaction was quenched with saturated aqueous NH₄Cl solution. The resulting mixture was extracted with ethyl acetate (3×100 mL). The combined organic layers were washed with water (2×100 mL) and brine (2×100 mL), dried over anhydrous Na₂SO₄, filtered and concentrated to dryness under reduced pressure to give ethyl (1S,3S,5S)-5-methyl-2-((5-phenylpentanoyl)glycyl)-2-azabicyclo[3.1.0]hexane-3-carboxylate (D; 98 mg, 65.1% yield) as a light-yellow oil, which was used directly without purification. LC/MS (ESI) m/z: 387 (M+H)⁺.

Step 3: (1S,3S,5S)-5-methyl-2-((5-phenylpentanoyl)glycyl)-2-azabicyclo[3.1.0]hexane-3-carboxylic acid (E). To a solution of ethyl (1 S,3S,5S)-5-methyl-2-((5-phenylpentanoyl)glycyl)-2-azabicyclo[3.1.0]hexane-3-carboxylate (D; 98 mg, 0.25 mmol) in methanol (1.5 mL) and water (0.5 mL) was added a solution of LiOH·H₂O (32 mg, 0.76 mmol) at 0° C. and the mixture was stirred at room temperature for 16 hours. The mixture was acidified with 1 N aq. HCl solution to pH<3 and concentrated to dryness under reduced pressure to give (1 S,3S,5S)-5-methyl-2-((5-phenylpentanoyl)glycyl)-2-azabicyclo[3.1.0]hexane-3-carboxylic acid (E; 90 mg, 90.9% yield) as a white solid, which was used directly in next step without further purification. LC/MS (ESI) m/z: 359 (M+H)⁺.

Step 4: (1S,3S,5S)-N-((R)-1-(4-carbamimidoylthiophen-2-yl)ethyl)-5-methyl-2-((5-phenyl-pentanoyl)glycyl)-2-azabicyclo[3.1.0]hexane-3-carboxamide (Compound 130). To a solution of (1S,3S,5S)-5-methyl-2-((5-phenylpentanoyl)glycyl)-2-azabicyclo[3.1.0]hexane-3-carboxylic acid (E; 43 mg, 0.116 mmol) and (R)-5-(1-aminoethyl)thiophene-3-carboximidamide (F; 19.6 mg, 0.116 mmol) in DMF (1 mL) was added DIPEA (90 mg, 0.69 mmol) and T₃P (220 mg, 0.35 mmol, 50% in ethyl acetate). The mixture was stirred at room temperature under N₂ atmosphere overnight. The reaction was quenched with saturated aqueous NH₄Cl solution. The resulting mixture was extracted with ethyl acetate (3×100 mL). The combined organic layers were washed with water (2×100 mL) and brine (2×100 mL), dried over anhydrous Na₂SO₄, filtered and concentrated to dryness under reduced pressure to give Compound 130 (1.6 mg, 2.5% yield) as a white solid. ¹H NMR (400 MHz, CD₃OD) δ 8.23 (dd, J=7.1, 1.6 Hz, 1H), 7.43 (t, J=11.3 Hz, 1H), 7.27-7.21 (m, 2H), 6.90 (dd, J=10.4, 4.6 Hz, 3H), 4.62-4.55 (m, 2H), 4.49-4.42 (m, 1H), 4.06-3.93 (m, 4H), 3.69 (dt, J=9.9, 6.3 Hz, 1H), 3.61 (dd, J=12.0, 4.7 Hz, 1H), 2.37-2.32 (m, 2H), 2.25-2.17 (m, 1H), 2.06-1.98 (m, 3H), 1.81 (dt, J=6.6, 3.4 Hz, 4H); LC/MS (ESI) m/z: 510 (M+H)⁺.

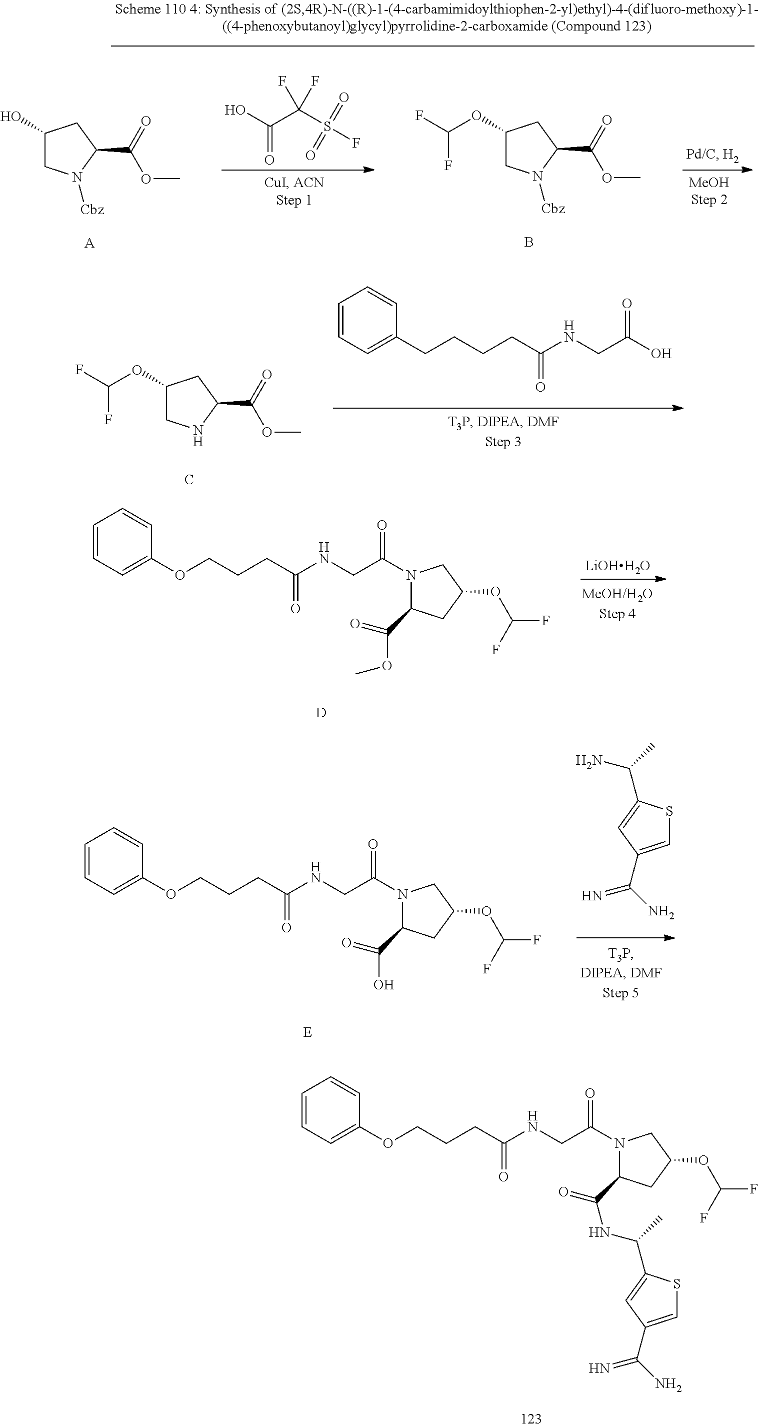

Step 1: 1-benzyl 2-methyl (2S,4R)-4-(difluoromethoxy)pyrrolidine-1,2-dicarboxylate (B). To a mixture of 1-benzyl 2-methyl (2S,4R)-4-hydroxypyrrolidine-1,2-dicarboxylate (A; 2 g, 7.17 mmol) in acetonitrile (20 mL) was added cuprous iodide (272 mg, 1.43 mmol) followed by a solution of 2,2-difluoro-2-(fluorosulfonyl)acetic acid (1.5 g, 8.6 mmol) in acetonitrile (6 mL) at 0° C., and the mixture was stirred at 50° C. for 16 hours. The mixture was diluted with acetonitrile and washed with brine, dried over anhydrous Na₂SO₄, filtered and concentrated to dryness under reduced pressure. The residue was purified by chromatography on silica gel (hexane:ethyl acetate=1:1) to give 1-benzyl 2-methyl (2S,4R)-4-(difluoromethoxy)pyrrolidine-1,2-dicarboxylate (B; 261 mg, yield 11.3%) as an oil. LC/MS (ESI) m/z: 330 (M+H)⁺.

Step 2: methyl (2S,4R)-4-(difluoromethoxy)pyrrolidine-2-carboxylate (C). To a solution of 1-benzyl 2-methyl (2S,4R)-4-(difluoromethoxy)pyrrolidine-1,2-dicarboxylate (B; 261 mg, 0.79 mmol) in methanol (2 mL) was added Pd/C (26 mg, 10% wt) at 0° C., and the reaction mixture was stirred at room temperature under H₂ atmosphere for 1 hour. The mixture was filtered and concentrated to dryness under reduced pressure to give methyl (2S,4R)-4-(difluoromethoxy)pyrrolidine-2-carboxylate (C; 143 mg, yield 92.8%) as an oil, which was used directly in the next step. LC/MS (ESI) m/z: 196 (M+H)⁺.

Step 3: methyl (2S,4R)-4-(difluoromethoxy)-1-((4-phenoxybutanoyl)glycyl)pyrrolidine-2-carboxylate (D). To a mixture of methyl (2S,4R)-4-(difluoromethoxy)pyrrolidine-2-carboxylate (C; 143 mg, 0.73 mmol) and (4-phenoxybutanoyl)glycine (174 mg, 0.73 mmol) in DMF (3 mL) was added DIPEA (0.7 mL, 4.38 mmol) followed by T₃P (1.4 g, 2.19 mmol, 50% in ethyl acetate) at 0° C., and the mixture was stirred at 30° C. for 16 hours. The mixture was washed with saturated aq.NaHCO₃, diluted with CHCl₃:IPA=3:1 and washed with brine, dried over anhydrous Na₂SO₄, filtered and concentrated to dryness under reduced pressure. The residue was purified by chromatography on silica gel (dichloromethane:methanol=20:1) to give methyl (2S,4R)-4-(difluoromethoxy)-1-((4-phenoxy-butanoyl)glycyl)pyrrolidine-2-carboxylate (D; 250 mg, yield 82.7%) as an oil. LC/MS (ESI) m/z: 415 (M+H)⁺.

Step 4: (2S,4R)-4-(difluoromethoxy)-1-((4-phenoxybutanoyl)glycyl)pyrrolidine-2-carboxylic acid (E). To a solution of methyl (2S,4R)-4-(difluoromethoxy)-1-((4-phenoxybutanoyl) glycyl)pyrrolidine-2-carboxylate (D; 250 mg, 0.6 mmol) in methanol (2 mL) and tetrahydrofuran (1 mL) was added a solution of LiOH (25 mg, 0.6 mmol) in H₂O (1 mL) at 0° C., and the mixture was stirred at 25° C. for 4 hours. The mixture was diluted with water and extracted with ethyl acetate twice. The water layer was acidified with 0.5 M aq. HCl solution, and extracted with ethyl acetate twice. The combined organic layers were washed with brine, dried over anhydrous Na₂SO₄, filtered and concentrated to dryness under reduced pressure to give (2S,4R)-4-(difluoromethoxy)-1-((4-phenoxybutanoyl)glycyl)pyrrolidine-2-carboxylic acid (E; 160 mg, yield 66.6%) as an oil, which was used directly in the next step. LC/MS (ESI) m/z: 401 (M+H)⁺

Step 5: (2S,4R)-N-((R)-1-(4-carbamimidoylthiophen-2-yl)ethyl)-4-(difluoromethoxy)-1-((4-phenoxybutanoyl)glycyl)pyrrolidine-2-carboxamide (Compound 123). To a mixture of compound E (70 mg, 0.18 mmol) and (R)-5-(1-aminoethyl)thiophene-3-carboximidamide (44 mg, 0.26 mmol) in DMF (1 mL) was added DIPEA (0.2 mL, 1.08 mmol) followed by T₃P (343 mg, 0.54 mmol, 50% in ethyl acetate) at 0° C., and the mixture was stirred at 30° C. for 16 hours. The mixture was washed with saturated aq.NaHCO₃ solution, diluted with CHCl₃:IPA=3:1 and washed with brine, dried over anhydrous Na₂SO₄, filtered and concentrated to dryness under reduced pressure. The residue was purified by prep-HPLC to give Compound 124 (10.1 mg, yield 10.2%) as a white solid. ¹H NMR (400 MHz, CD₃OD) δ 8.22 (dd, J=3.6, 3.6 Hz, 1H), 7.56-7.50 (m, 1H), 7.26-7.20 (m, 2H), 6.91-6.87 (m, 3H), 6.68-6.27 (m, 1H), 5.34-5.21 (m, 1H), 4.98-4.94 (m, 1H), 4.61-4.52 (m, 1H), 4.07 (d, J=16.8 Hz, 1H), 4.01-3.95 (m, 3H), 3.91-3.85 (m, 1H), 3.80-3.67 (m, 1H), 2.50-2.41 (m, 3H), 2.26-2.15 (m, 1H), 2.11-2.04 (m, 2H), 1.60 (dd, J=23.2, 23.2 Hz, 3H); LC/MS (ESI) m/z: 552 (M+H)⁺.

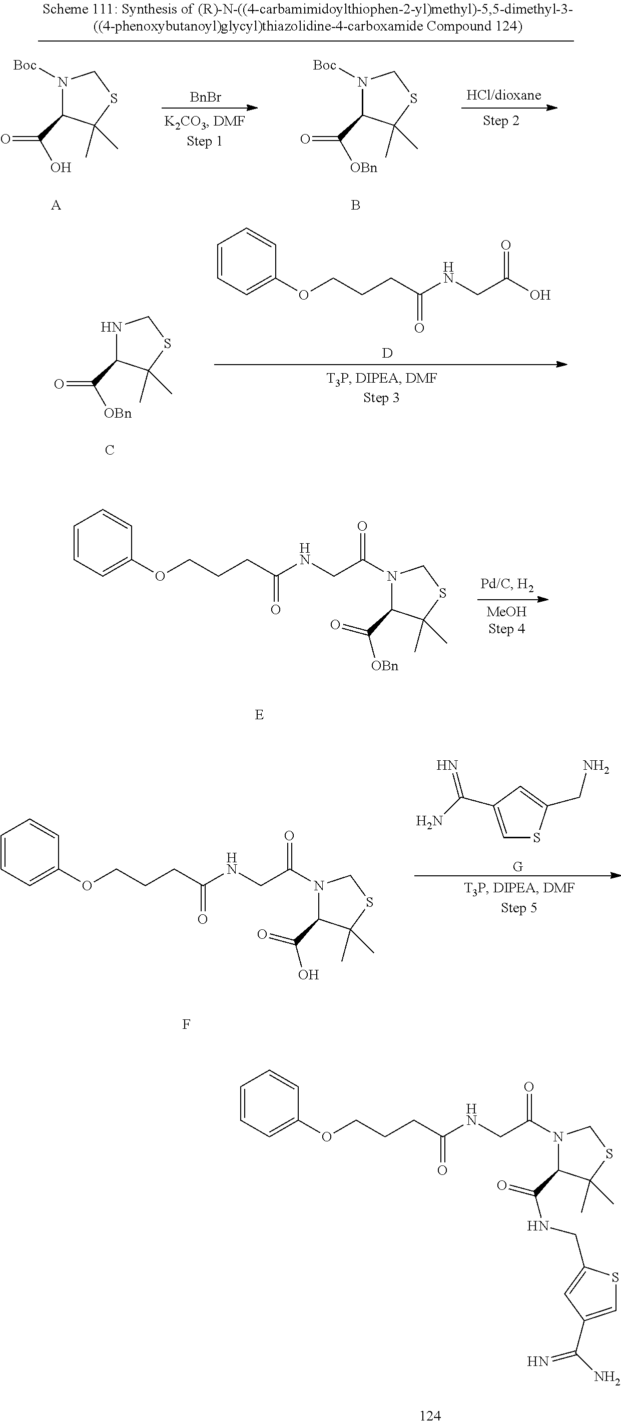

Step 1: 4-benzyl 3-(tert-butyl) (R)-5,5-dimethylthiazolidine-3,4-dicarboxylate (B). To a solution of (R)-3-(tert-butoxycarbonyl)-5,5-dimethylthiazolidine-4-carboxylic acid (A; 50 mg, 0.19 mol) in DMF (3 mL) was added BnBr (0.03 mL, 0.23 mol) and K₂CO₃ (79 mg, 0.57 mol) at 0° C. under N₂ atmosphere and the mixture was stirred at 25° C. for 16 hours. The mixture was diluted with H₂O and extracted with EtOAc twice and washed with aq. NH₄Cl solution. The organic layer was dried over anhydrous Na₂SO₄, filtered and concentrated to dryness under reduced pressure. The residue was purified by column chromatography on silica gel (PE:EtOAc=9:1) to give 4-benzyl 3-(tert-butyl) (R)-5,5-dimethylthiazolidine-3,4-dicarboxylate (B; 85 mg, yield 67.2%) as a colorless oil. LC/MS (ESI) m/z: 352(M+H)⁺.

Step 2: benzyl (R)-5,5-dimethylthiazolidine-4-carboxylate (C). To a solution of 4-benzyl 3-(tert-butyl) (R)-5,5-dimethylthiazolidine-3,4-dicarboxylate (B; 85 mg, 0.24 mmol) in HCl/1,4-dioxane (6 mL) at 25° C. and the mixture was stirred at 25° C. for 2 hours. The reaction mixture was concentrated to dryness under reduced pressure, diluted with DCM again and dried under vacuum to give benzyl (R)-5,5-dimethylthiazolidine-4-carboxylate (C; 58 mg, yield 96.7%) as a white solid, which was used directly in the next step without further purification. LC/MS (ESI) m/z: 252(M+H)⁺.

Step 3: benzyl (R)-5,5-dimethyl-3-((4-phenoxybutanoyl)glycyl)thiazolidine-4-carboxylate (E). To a mixture of benzyl (R)-5,5-dimethylthiazolidine-4-carboxylate (C; 80 mg, 0.32 mmol) and (4-phenoxybutanoyl)glycine (D; 75 mg, 0.35 mmol) in DMF (3 mL) was added DIPEA (0.32 mL, 1.92 mmol) and T₃P (608 mg, 0.96 mmol) at 0° C. under N₂ atmosphere and the mixture was stirred at 40° C. for 48 hours. The mixture was diluted with water and extracted with EtOAc twice. The combined organic layers were dried over anhydrous Na₂SO₄, filtered and concentrated to dryness under reduced pressure. The residue was purified by column chromatography on silica gel (DCM:MeOH=96:4) to give benzyl (R)-5,5-dimethyl-3-((4-phenoxybutanoyl)glycyl)thiazolidine-4-carboxylate (E; 80 mg, yield 53.3%) as a yellow oil. LC/MS (ESI) m/z: 471(M+H)⁺.

Step 4: (R)-5,5-dimethyl-3-((4-phenoxybutanoyl)glycyl)thiazolidine-4-carboxylic acid (F). To a solution of benzyl (R)-5,5-dimethyl-3-((4-phenoxybutanoyl)glycyl)thiazolidine-4-carboxylate (E; 80 mg, 0.17 mmol) in MeOH (5 mL) was added Pd/C (20 mg, 10% wt) and degassed under H₂ atmosphere three times and the reaction mixture was stirred under a H₂ balloon at room temperature for 2 hours. The mixture was filtered and filtrate was concentrated to dryness under reduced pressure to dryness to give (R)-5,5-dimethyl-3-((4-phenoxybutanoyl)glycyl)thiazolidine-4-carboxylic acid (F; 40 mg, yield 61.5%) as a colorless oil. The crude product was used directly in next step without further purification. LC/MS (ESI) (m/z): 381(M+H)⁺.

Step 5: (R)-N-((4-carbamimidoylthiophen-2-yl)methyl)-5,5-dimethyl-3-((4-phenoxybutanoyl) glycyl)thiazolidine-4-carboxamide (Compound 124). To a mixture of (R)-5,5-dimethyl-3-((4-phenoxybutanoyl)glycyl)thiazolidine-4-carboxylic acid (F; 40 mg, 0.11 mmol) and 5-(aminomethyl)-thiophene-3-carboximidamide (G; 25 mg, 0.17 mmol) in DMF (3 mL) was added DIPEA (0.08 mL, 0.66 mmol) and T₃P (200 mg, 0.33 mmol) at 0° C. under N₂ atmosphere and the mixture was stirred at 25° C. for 3 hours. The mixture was diluted with water and extracted with CHCl₃/i-PrOH(V/V=3:1) ten times. The combined organic layers were dried over anhydrous Na₂SO₄, filtered and concentrated to dryness under reduced pressure. The residue was purified by prep-HPLC to give Compound 124 (10 mg, yield 18.5%) as a white solid. ¹H NMR (400 MHz, CD₃OD) δ 8.22 (t, J=2.7 Hz, 1H), 7.42 (s, 1H), 7.24 (t, J=8.0 Hz, 2H), 6.89 (dd, J=7.6, 3.9 Hz, 4H), 4.83-4.80 (m, 2H), 4.56 (d, J=3.9 Hz, 2H), 4.34 (s, 1H), 4.11 (dd, J=35.0, 16.9 Hz, 2H), 4.02-3.98 (m, 2H), 2.47 (t, J=7.1 Hz, 2H), 2.08 (dd, J=13.7, 6.6 Hz, 2H), 1.56 (d, J=3.4 Hz, 3H), 1.37 (d, J=5.5 Hz, 3H); LC/MS (ESI) m/z: 518(M+H)⁺.

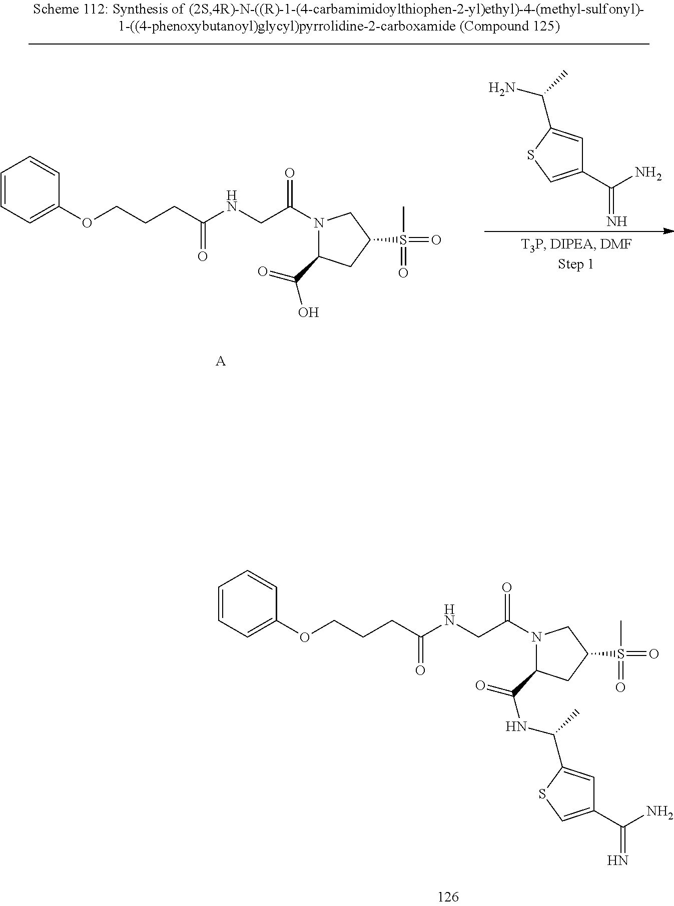

Step 1: (2S,4R)-N-((R)-1-(4-carbamimidoylthiophen-2-yl)ethyl)-4-(methylsulfonyl)-1-((4-phenoxybutanoyl)glycyl)pyrrolidine-2-carboxamide (Compound 125). To a mixture of compound A (30 mg, 0.07 mmol) and (R)-5-(1-aminoethyl)thiophene-3-carboximidamide (18 mg, 0.11 mmol) in DMF (1 mL) was added DIPEA (0.07 mL, 0.42 mmol) followed by T₃P (134 mg, 0.21 mmol, 50% wt in ethyl acetate) at 0° C., and the mixture was stirred at 30° C. for 16 hours. The mixture was washed with saturated aq. NaHCO₃ solution, diluted with CHCl₃:IPA=3:1 and washed with brine, dried over anhydrous Na₂SO₄, filtered and concentrated to dryness under reduced pressure. The residue was purified by prep-HPLC to give Compound 125 (2.7 mg, yield 6.9%) as a white solid. ¹H NMR (400 MHz, CD₃OD) δ 8.51 (s, 1H), 8.22 (s, 1H), 7.50 (s, 1H), 7.23 (t, J=8.0 Hz, 2H), 6.89 (dd, J=6.4, 8.8 Hz, 3H), 5.33-5.18 (m, 1H), 4.67 (dd, J=8.4, 8.4 Hz, 1H), 4.12-4.05 (m, 4H), 4.00 (t, J=6.2 Hz, 2H), 3.87-3.71 (m, 1H), 3.04 (d, J=14.0 Hz, 3H), 2.86-2.68 (m, 1H), 2.48-2.42 (m, 2H), 2.11-2.04 (m, 2H), 1.57 (d, J=7.0 Hz, 3H), 1.05-0.98 (m, 1H); LC/MS (ESI) m/z: 564 (M+H)⁺.

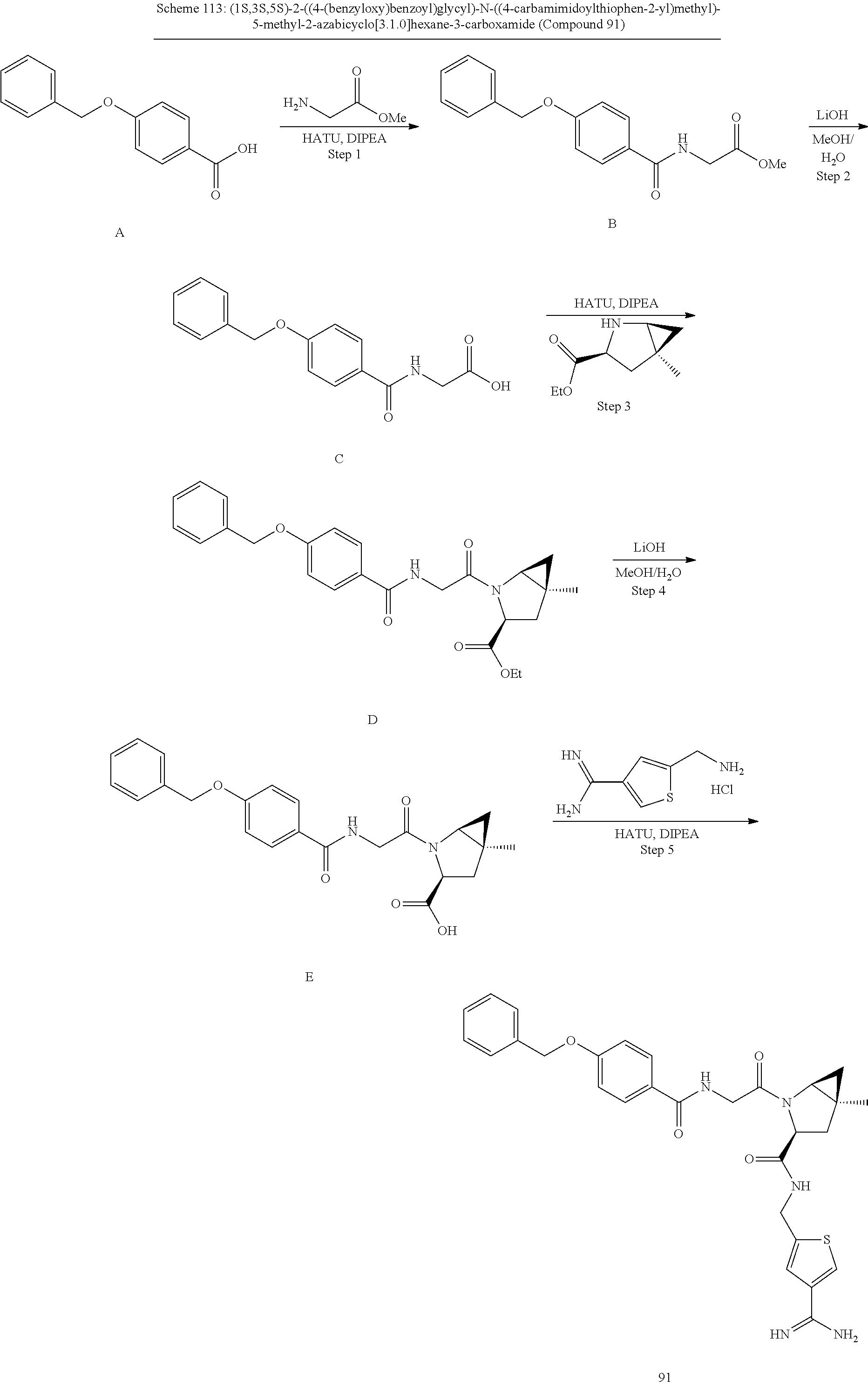

Step 1: methyl 2-{[4-(benzyloxy)phenyl]formamido}acetate (B). To a solution of 4-(benzyloxy)benzoic acid (A; 1.0 g, 3.78 mmol, 1.0 equiv.) in DMF (5 mL) was added, methyl 2-aminoacetate (0.52 g, 4.16 mmol, 1.1 equiv.), HATU (1.87 g, 4.91 mmol, 1.3 equiv.), and finally DIPEA (1.98 mL, 11.33 mmol, 3.0 equiv.). The reaction was stirred for 20 minutes and then diluted with water (5 mL). The solid was collected by filtration and then dried to obtain compound B as a white solid (1.0 g, 3.34 mmol, Yield 88.44%). LC/MS (ESI) m/z: 314 (M+H)⁺.

Step 2: {[4-(benzyloxy)phenyl]formamido}acetic acid (C). To a solution of compound B (0.2 g, 0.67 mmol, 1.0 equiv.) in MeOH (6 mL) and H₂O (2 mL) was added LiOH (0.08 g, 3.34 mmol, 5 equiv.). The reaction was stirred at room temperature for 1 h, concentrated to remove MeOH, and then diluted with EtOAc (10 mL). The pH was adjusted to 1 using 1 N HCl. The two layers were separated, and the aqueous layer was extracted with EtOAc. The combined organic extracts were washed with brine, dried over Na₂SO₄ and concentrated to give compound C as a white solid (0.17 g, 0.55 mmol, Yield 82.88%). LC/MS (ESI) m/z: 286 (M+(H)H.

Step 3: ethyl (1R,2S,5S)-3-(2-{[4-(benzyloxy)phenyl]formamido}acetyl)-1-methyl-3-azabicyclo[3.1.0]hexane-2-carboxylate (D). To a solution of compound C (0.07 g, 0.25 mmol, 1.0 equiv.) in DMF (4 mL) was added ethyl (1 S,3S,5S)-5-methyl-2-azabicyclo[3.1.0]hexane-3-carboxylate (0.046 g, 0.27 mmol, 1.1 equiv.), HATU (0.121 g, 0.32 mmol, 1.3 equiv.) and DIPEA (0.086 mL, 0.49 mmol, 3.0 equiv.). The reaction was stirred at room temperature for 30 minutes. Water (2 mL) and CH₂Cl₂ (5 mL) were added. The two layers were separated, and the aqueous layer was extracted with CH₂Cl₂ (2 mL×3), washed with brine, dried over Na₂SO₄, and concentrated. Purification by Combi-Flash; 12 g column, solvent A=CH₂Cl₂, solvent B=MeOH, 100% A to 5% B gave compound D as a sticky yellow oil (0.07 g, Yield 65.36%). LC/MS (ESI) m/z: 437 (M+H)⁺.

Step 4: (1R,2S,5S)-3-(2-{[4-(benzyloxy)phenyl]formamido}acetyl)-1-methyl-3-azabicyclo[3.1.0]-hexane-2-carboxylic acid (E). To a solution of compound 4 (0.07 g, 0.17 mmol, 1.0 equiv.) in MeOH (3 mL) and H₂O (1 mL) was added LiOH (0.008 g, 0.33 mmol, 2.0 equiv.). The reaction was stirred at room temperature for 5 hours, concentrated to remove MeOH, and then diluted with CH₂Cl₂ (3 mL). The pH was adjusted to 1 using 1 N HCl. The two layers were separated, and the aqueous layer was extracted with CH₂Cl₂ (2 mL×3). The combined organic extracts were washed with brine, dried over Na₂SO₄ and concentrated to give compound E (0.055 g, 0.14 mmol, Yield 81.27%) as a yellow solid. LC/MS (ESI) m/z: 409 (M+H)⁺.

Step 5: (1R,2S,5S)-3-(2-{[4-(benzyloxy)phenyl]formamido}acetyl)-N-[(4-carbamimidoyl-thiophen-2-yl)methyl]-1-methyl-3-azabicyclo[3.1.0]hexane-2-carboxamide (Compound 91). To a solution of compound E (0.04 g, 0.098 mmol, 1.0 equiv.) in DMF (1 mL) was added 5-(aminomethyl)-thiophene-3-carboximidamide (0.02 g, 0.11 mmol, 1.1 equiv.), HATU (0.04 g, 0.11 mmol, 1.1 equiv.), and DIPEA (0.05 mL, 0.29 mmol, 3.0 equiv.). The reaction mixture was stirred at room temperature for 10 minutes and then purified directly by HPLC to give Compound 91 (0.02 g, 0.033 mmol, Yield 24.75%). LC/MS (ESI) m/z: 546 (M+H)⁺. ¹H NMR (400 MHz, DMSO-d₆) δ 8.55 (dt, J=19.0, 5.8 Hz, 2H), 8.45 (s, 1H), 8.36-8.29 (m, 1H), 7.83 (dd, J=9.0, 2.5 Hz, 2H), 7.55-7.30 (m, 4H), 7.08 (dd, J=9.1, 2.8 Hz, 2H), 5.17 (s, 2H), 4.71-4.57 (m, 1H), 4.50-4.40 (m, 1H), 4.43-4.33 (m, 1H), 4.31 (dd, J=16.6, 5.6 Hz, 1H), 4.02 (td, J=16.2, 5.6 Hz, 1H), 3.43 (dd, J=6.1, 2.4 Hz, 1H), 2.28 (t, J=12.3 Hz, 1H), 2.00 (dd, J=13.2, 3.1 Hz, 1H), 1.25-1.08 (m, 4H), 0.70-0.58 (m, 1H).

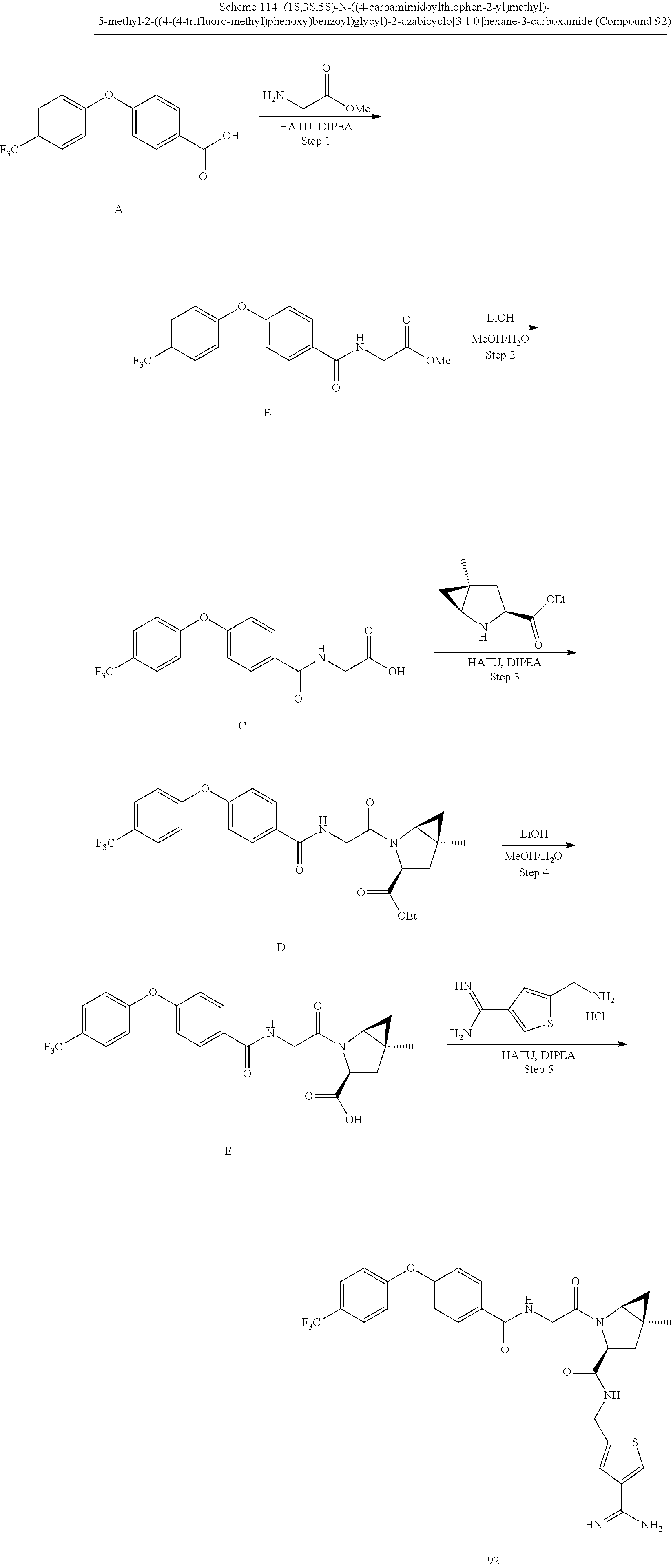

Step 1: methyl 2-({4-[4-(trifluoromethyl)phenoxy]phenyl}formamido)acetate (B). To a solution of 4-[4-(trifluoromethyl)phenoxy]benzoic acid (A; 1.0 g, 3.78 mmol, 1.0 equiv.) in DMF (5 mL) was added, methyl 2-aminoacetate (0.39 g, 3.12 mmol, 1.1 equiv.), HATU (1.40 g, 3.69 mmol, 1.3 equiv.), and finally DIPEA (0.99 mL, 5.67 mmol, 2.0 equiv.). The reaction was stirred for 20 minutes and then diluted with water (5 mL). The solid was collected by filtration and then dried to obtain compound B as a white solid (0.91 g, 2.56 mmol, Yield 90.87%). LC/MS (ESI) m/z: 354 (M+H)⁺.

Step 2: (4-(4-(trifluoromethyl)phenoxy)benzoyl)glycine (C). To a solution of compound B (0.9 g, 2.55 mmol, 1.0 equiv.) in MeOH (9 mL) and H₂O (3 mL) was added LiOH (0.31 g, 12.74 mmol, 5.0 equiv.). The reaction was stirred at room temperature for 2 hours, concentrated to remove MeOH, and then diluted with EtOAc (10 mL). The pH was adjusted to 1 using 1 N HCl. The two layers were separated, and the aqueous layer was extracted with EtOAc (5 mL×3). The combined organic extracts were washed with brine, dried over Na₂SO₄ and concentrated to give compound C as a white solid (0.8 g, 2.36 mmol, Yield 92.57%). LC/MS (ESI) m/z: 340 (M+H)⁺.

Step 3: ethyl (1S,3S,5S)-5-methyl-2-[2-({4-[4-(trifluoromethyl) phenoxy]phenyl}formamido) acetyl]-2-azabicyclo[3.1.0]hexane-3-carboxylate (D). To a solution of compound C (0.05 g, 0.147 mmol, 1.0 equiv.) in DMF (1 mL) was added ethyl (1S,3S,5S)-5-methyl-2-azabicyclo[3.1.0]hexane-3-carboxylate (0.023 g, 0.13 mmol, 1.1 equiv.), HATU (0.07 g, 0.17 mmol, 1.3 equiv.) and DIPEA (0.07 mL, 0.40 mmol, 3.0 equiv.). The reaction was stirred at room temperature for 20 minutes. Water (1 mL) and CH₂Cl₂ (2 mL) were added. The two layers were separated, and the aqueous layer was extracted with CH₂Cl₂, washed with brine, dried over Na₂SO₄, and concentrated. Purification by CombiFlash; 12 g column, solvent A=CH₂Cl₂, solvent B=MeOH, 100% A to 5% B gave compound D as a sticky yellow oil (0.06 g, 0.122 mmol, Yield 91.31%). LC/MS (ESI) m/z: 491 (M+H)⁺.

Step 4: (1S,3S,5S)-5-methyl-2-[2-({4-[4-(trifluoromethyl)phenoxy]phenyl}formamido)-acetyl]-2-azabicyclo[3.1.0]hexane-3-carboxylic acid (E). To a solution of compound D (0.05 g, 0.10 mmol, 1.0 equiv.) in MeOH (3 mL) and H₂O (1 mL) was added LiOH (0.005 g, 0.20 mmol, 2.0 equiv.). The reaction was stirred at room temperature for 5 hours, concentrated to remove MeOH, and then diluted with CH₂Cl₂ (3 mL). The pH was adjusted to 1 using 1 N HCl. The two layers were separated, and the aqueous layer was extracted with CH₂Cl₂ (2 mL×3). The combined organic extracts were washed with brine, dried over Na₂SO₄ and concentrated to give compound E (0.045 g, 0.1 mmol, Yield 95.46%) as a yellow solid. LC/MS (ESI) m/z: 463 (M+H)⁺.

Step 5: (1S,3S,5S)-N-((4-carbamimidoylthiophen-2-yl)methyl)-5-methyl-2-((4-(4-(trifluoro-methyl)phenoxy)benzoyl)glycyl)-2-azabicyclo[3.1.0]hexane-3-carboxamide (Compound 92). To a solution of compound E (0.045 g, 0.098 mmol, 1.0 equiv.) in DMF (1 mL) was added 5-(aminomethyl)-thiophene-3-carboximidamide (0.021 g, 0.107 mmol, 1.1 equiv.), HATU (0.041 g, 0.107 mmol, 1.1 equiv.), and DIPEA (0.051 mL, 0.292 mmol, 3.0 equiv.). The reaction mixture was stirred at room temperature for 10 minutes and then purified directly by HPLC to give Compound 92 (0.02 g, 0.033 mmol, Yield 24.75%). LC/MS (ESI) m/z: 600 (M+H)⁺. ¹H NMR (400 MHz, DMSO-d₆) δ 8.71 (q, J=11.0, 8.4 Hz, 1H), 8.55 (t, J=5.9 Hz, 1H), 8.44 (s, 1H), 8.37-8.29 (m, 1H), 7.99-7.91 (m, 2H), 7.78 (d, J=8.5 Hz, 2H), 7.48 (d, J=15.7 Hz, 1H), 4.67 (dd, J=11.4, 3.0 Hz, 1H), 4.46 (dd, J=13.9, 7.7 Hz, 1H), 4.44-4.31 (m, 2H), 4.11-3.98 (m, 1H), 3.44 (dd, J=5.9, 2.4 Hz, 1H), 2.29 (t, J=12.6 Hz, 1H), 2.00 (dd, J=13.0, 3.2 Hz, 1H), 1.22 (d, J=11.3 Hz, 4H), 0.62-0.70 (m, 1H).

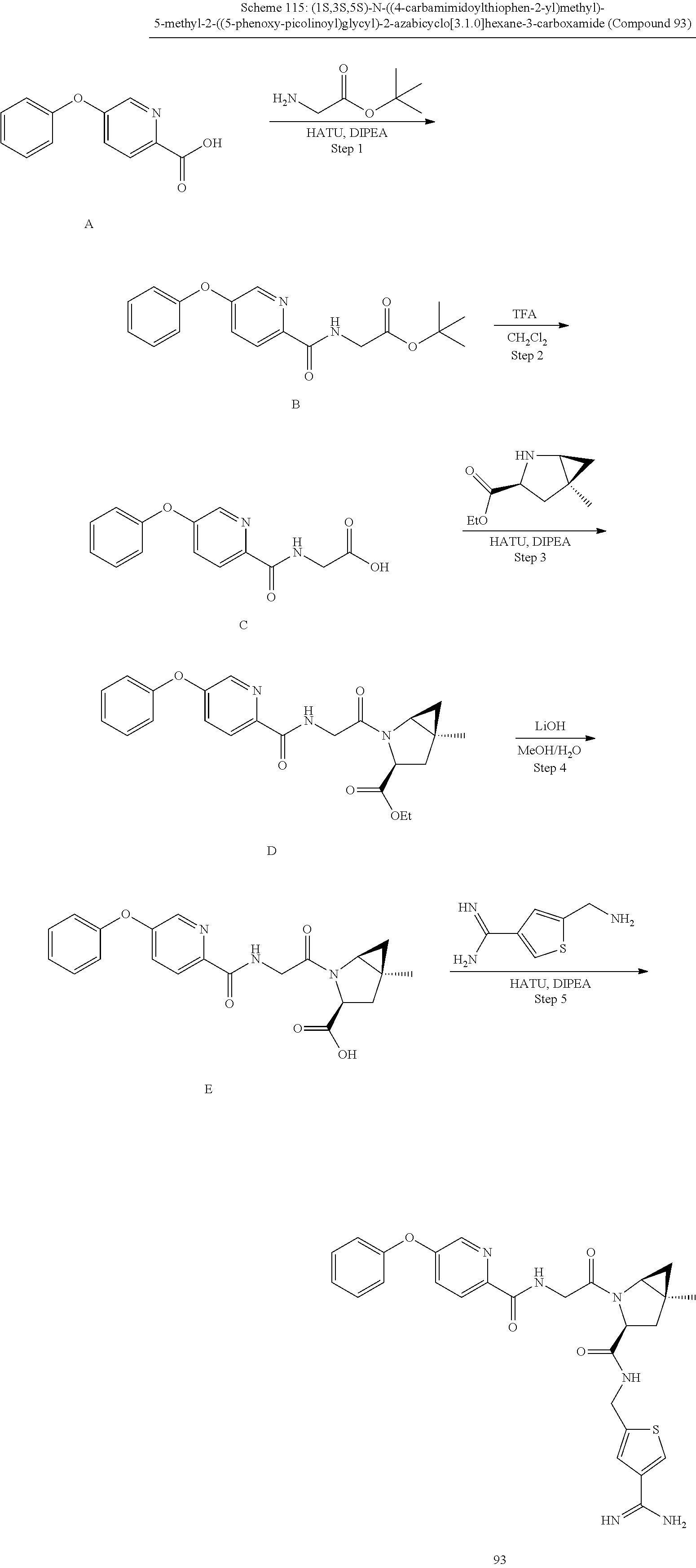

Step 1: tert-butyl (5-phenoxypicolinoyl)glycinate (B). To a solution of 5-phenoxypyridine-2-carboxylic acid (A; 0.25 g, 1.16 mmol, 1.0 equiv.) in DMF (2 mL) was added, tert-butyl 2-aminoacetate (0.23 g, 1.39 mmol, 1.2 equiv.), HATU (0.57 g, 1.51 mmol, 1.3 equiv.), and finally DIPEA (0.61 mL, 3.49 mmol, 3 equiv.). The reaction was stirred for 20 minutes at room temperature and then diluted with water (2 mL). The solid was collected by filtration, washed with water and then dried to obtain compound B (0.35 g, 1.07 mmol, Yield 91.75%). LC/MS (ESI) m/z: 329 (M+H)⁺.

Step 2: (5-phenoxypicolinoyl)glycine (3). TFA (0.82 mL) was added to a solution of compound B in CH₂Cl₂ (2 mL) at ice-bath temperature. The reaction was stirred at room temperature for 2 hours and then concentrated to dryness to give compound C (0.25 g, 0.92 mmol, Yield 86.15%) as a white solid. LC/MS (ESI) m/z: 273 (M+H)⁺.

Step 3: ethyl (1S,3S,5S)-5-methyl-2-((5-phenoxypicolinoyl)glycyl)-2-azabicyclo[3.1.0]-hexane-3-carboxylate (D). To a solution of compound C (0.08 g, 0.28 mmol, 1.2 equiv.) in DMF (2 mL) was added ethyl (1 S,3S,5S)-5-methyl-2-azabicyclo[3.1.0]hexane-3-carboxylate (0.04 g, 0.24 mmol, 1.0 equiv.), HATU (0.12 g, 0.31 mmol, 1.3 equiv.) and DIPEA (0.08 mL, 0.47 mmol, 2.0 equiv.). The reaction was stirred at room temperature for 30 minutes. Water (4 mL) and CH₂Cl₂ (5 mL) were added. The two layers were separated, and the aqueous layer was extracted with CH₂Cl₂, washed with brine, dried over Na₂SO₄, and concentrated. Purification by CombiFlash; 4 g column, solvent A=CH₂Cl₂, solvent B=MeOH, 100% A to 5% B gave compound D (0.1 g, 0.236 mmol, Yield 99.90%). LC/MS (ESI) m/z: 424 (M+H)⁺.

Step 4: (1S,3S,5S)-5-methyl-2-((5-phenoxypicolinoyl)glycyl)-2-azabicyclo[3.1.0]hexane-3-carboxylic acid (E). To a solution of compound D (0.1 g, 0.236 mmol, 1.0 equiv.) in MeOH (3 mL) and H₂O (1 mL) was added LiOH (0.011 g, 0.472 mmol, 2.0 equiv.). The reaction was stirred at room temperature for 4 hours, concentrated to remove MeOH, and then diluted with CH₂Cl₂ (3 mL). The pH was adjusted to 1 using 1 N HCl. The two layers were separated, and aqueous layer was extracted with CH₂Cl₂ (2 mL×3). The combined organic extracts were washed with brine, dried over Na₂SO₄ and concentrated to give compound E (0.085 g, 0.215 mmol, Yield 91.03%) as a white solid. LC/MS (ESI) m/z: 396 (M+H)⁺.

Step 5: (1S,3S,5S)-N-((4-carbamimidoylthiophen-2-yl)methyl)-5-methyl-2-((5-phenoxy-picolinoyl)glycyl)-2-azabicyclo[3.1.0]hexane-3-carboxamide (Compound 93). To a solution of compound 5 (0.03 g, 0.076 mmol, 1.0 equiv.) in DMF (2 mL) was added 5-(aminomethyl)thiophene-3-carboximidamide·HCl (0.017 g, 0.09 mmol, 1.2 equiv.), HATU (0.03 g, 0.08 mmol, 1.1 equiv.), and DIPEA (0.04 mL, 0.23 mmol, 3.0 equiv.). The reaction mixture was stirred at room temperature for 20 minutes and then purified directly by HPLC to give Compound 93 (0.018 g, 0.034 mmol, Yield 44.54%). LC/MS (ESI) m/z: 533 (M+H)⁺. ¹H NMR (400 MHz, DMSO-d₆) δ 8.64 (dt, J=28.3, 5.5 Hz, 2H), 8.50-8.39 (m, 2H), 8.36 (dd, J=5.3, 1.4 Hz, 1H), 8.06 (dd, J=8.6, 5.2 Hz, 1H), 7.58-7.45 (m, 2H), 7.29 (t, J=7.5 Hz, 1H), 7.24-7.16 (m, 2H), 4.70 (dd, J=11.4, 3.1 Hz, 1H), 4.53-4.34 (m, 3H), 4.15 (ddd, J=37.8, 16.9, 5.2 Hz, 1H), 3.44 (dd, J=6.0, 2.4 Hz, 1H), 2.32 (t, J=12.2 Hz, 1H), 2.13-1.94 (m, 1H), 1.28-1.13 (m, 4H), 0.62-0.70 (m, 1H).

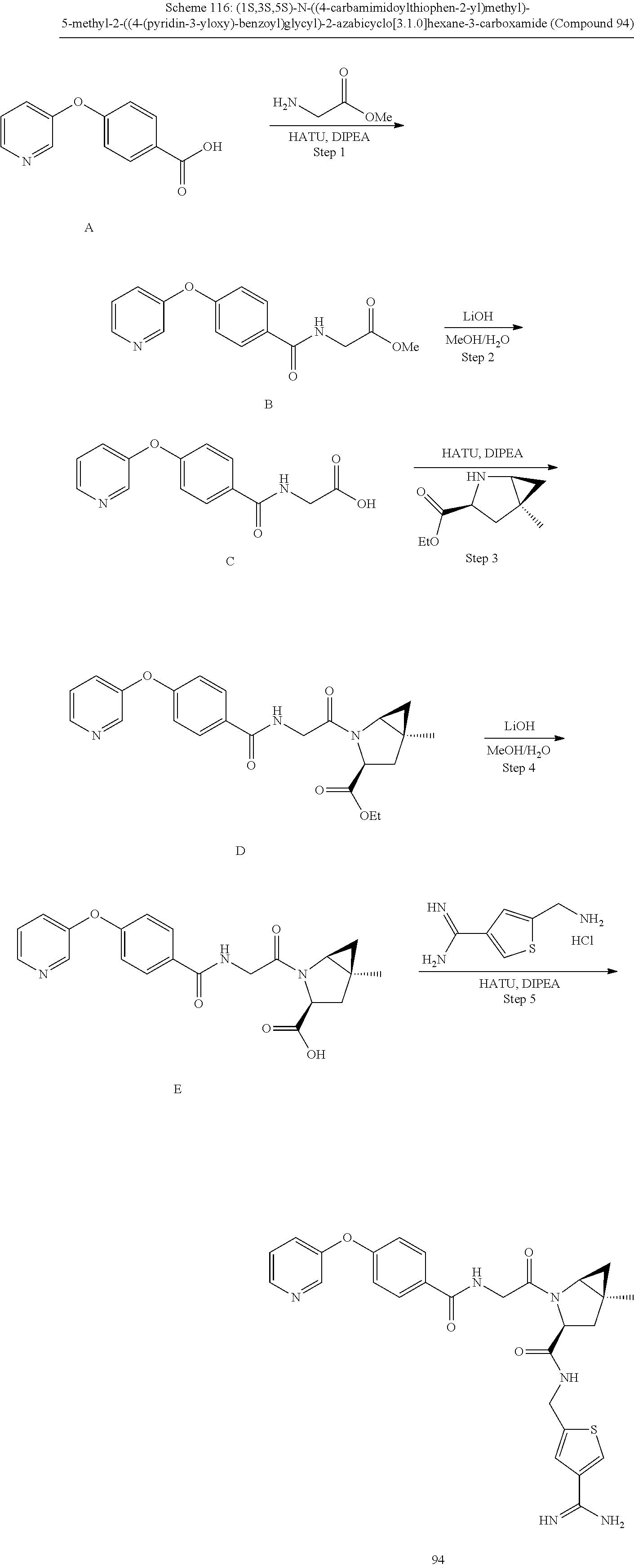

Step 1: methyl 2-{[4-(pyridin-3-yloxy)phenyl]formamido}acetate (B). To a solution of 4-(pyridin-3-yloxy)benzoic acid (A; 0.5 g, 2.32 mmol, 1.0 equiv.) in DMF (5 mL) was added, methyl 2-aminoacetate (0.35 g, 2.79 mmol, 1.1 equiv.), HATU (1.15 g, 3.02 mmol, 1.3 equiv.), and finally DIPEA (1.01 mL, 5.81 mmol, 3.0 equiv.). The reaction was stirred for 20 minutes and then diluted with water (2 mL) and EtOAc (5 mL). The two layers were separated, and the aqueous layer was extracted with EtOAc (5 mL×3). The combined organic extracts were washed with brine, dried over Na₂SO₄ and concentrated. Purification by CombiFlash; 24 g column, solvent A=CH₂Cl₂, solvent B=MeOH, 100% A to 5% B gave compound B as an off-white solid (0.54 g, 1.89 mmol, Yield 81.19%). LC/MS (ESI) m/z: 287 (M+H)⁺.

Step 2: {[4-(pyridin-3-yloxy)phenyl]formamido}acetic acid (C). To a solution of compound B (0.4 g, 1.4 mmol, 1.0 equiv.) in MeOH (6 mL) and H₂O (2 mL) was added LiOH (0.17 g, 6.99 mmol, 5 equiv.). The reaction was stirred at room temperature for 2 hours, concentrated to remove MeOH, and then diluted with CH₂Cl₂ (10 mL). The pH was adjusted to 3 using 1 N HCl. The product crashed out of solution and was filtered and then dried to obtain compound C as a white powder (0.29 g, 1.07 mmol, Yield 76.24%). LC/MS (ESI) m/z: 273 (M+H)⁺.

Step 3: ethyl (1S,3S,5S)-5-methyl-2-(2-{[4-(pyridin-3-yloxy)phenyl]formamido}acetyl)-2-azabicyclo[3.1.0]hexane-3-carboxylate (D). To a solution of compound C (0.07 g, 0.26 mmol, 1.0 equiv.) in DMF (2 mL) was added ethyl (1 S,3S,5S)-5-methyl-2-azabicyclo[3.1.0]hexane-3-carboxylate (0.05 g, 0.28 mmol, 1.1 equiv.), HATU (0.13 g, 0.33 mmol, 1.3 equiv.), and DIPEA (0.11 mL, 0.64 mmol, 2.5 equiv.). The reaction was stirred at room temperature for 10 minutes. Water (2 mL) and CH₂Cl₂ (5 mL) were added. The two layers were separated, and the aqueous layer was extracted with CH₂Cl₂, washed with brine, dried over Na₂SO₄, and concentrated. Purification by Comb Flash, 4 g column, solvent A=CH₂Cl₂, solvent B=MeOH, 100% A to 5% B gave compound D. (0.07 g, 0.165 mmol, Yield 64.29%). LC/MS (ESI) m/z: 424 (M+H)⁺.

Step 4: (1S,3S,5S)-5-methyl-2-(2-{[4-(pyridin-3-yloxy)phenyl]formamido}acetyl)-2-azabicyclo[3.1.0]hexane-3-carboxylic acid (E). To a solution of compound D (0.07 g, 0.17 mmol, 1.0 equiv.) in MeOH (3 mL) and H₂O (1 mL) was added LiOH (0.008 g, 0.33 mmol, 2 equiv.). The reaction was stirred at room temperature for 5 h, concentrated to remove MeOH, and then diluted with CH₂Cl₂ (3 mL). The pH was adjusted to 1 using 1 N HCl. The two layers were separated, and aqueous layer was extracted with CH₂Cl₂ (2 mL×3). The combined organic extracts were washed with brine, dried over Na₂SO₄ and concentrated. Purification by CombiFlash; 4 g column, solvent A=CH₂Cl₂, solvent B=MeOH, 100% A to 10% B compound E (0.06 g, 0.15 mmol, Yield 91.80%) as a white solid. LC/MS (ESI) m/z: 396 (M+H)⁺.

Step 5: (1S,3S,5S)-N-[(4-carbamimidoylthiophen-2-yl)methyl]-5-methyl-2-(2-{[4-(pyridin-3-yloxy)phenyl]formamido}acetyl)-2-azabicyclo[3.1.0]hexane-3-carboxamide (Compound 94). A solution of compound E (0.06 g, 0.08 mmol, 1.0 equiv.) in DMF (2 mL) was added 5-(aminomethyl)-thiophene-3-carboximidamide (0.032 g, 0.167 mmol, 1.1 equiv.), HATU (0.06 g, 0.17 mmol, 1.1 equiv.), and DIPEA (0.08 mL, 0.46 mmol, 3.0 equiv.). The reaction mixture was stirred at room temperature for 20 minutes and then purified directly by HPLC to give Compound 94 (0.02 g, 0.038 mmol, Yield 24.75%). LC/MS (ESI) m/z: 533 (M+H)⁺. ¹H NMR (400 MHz, DMSO-d₆) δ 8.71 (t, J=5.7 Hz, 1H), 8.56 (t, J=5.9 Hz, 1H), 8.46 (dd, J=4.8, 1.7 Hz, 4H), 8.36 (d, J=9.0 Hz, 1H), 8.00-7.90 (m, 3H), 7.61-7.46 (m, 2H), 7.13 (dd, J=8.9, 2.3 Hz, 2H), 4.07 (dd, J=16.5, 5.6 Hz, 1H), 3.49-3.43 (m, 1H), 2.91 (s, 1H), 2.71 (m, 2H), 2.31 (t, J=12.5 Hz, 1H), 2.02 (dd, J=12.8, 3.4 Hz, 1H), 1.24 (d, J=10.9 Hz, 4H), 0.62-0.70 (m, 1H).

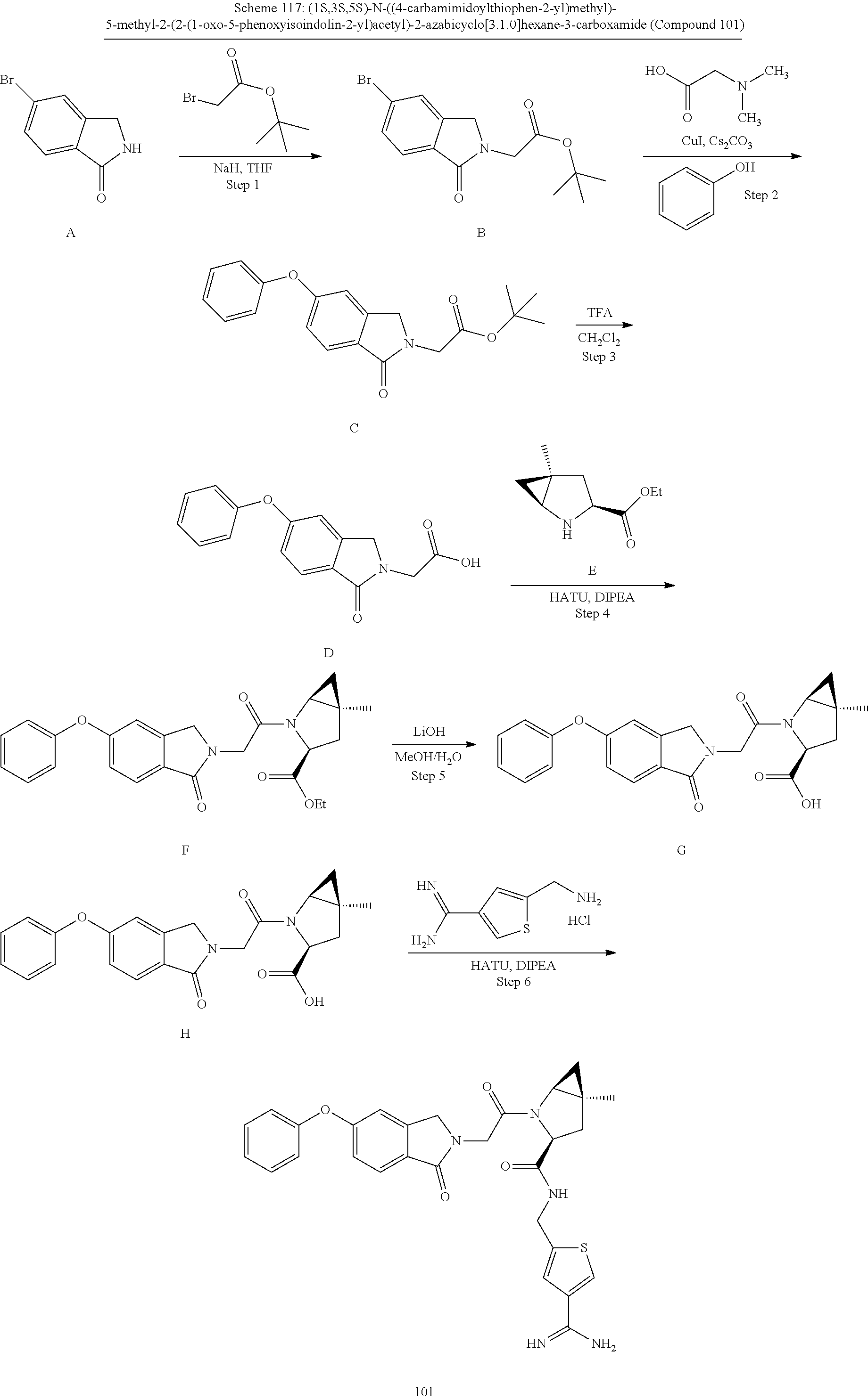

Step 1: tert-butyl 2-(5-bromo-1-oxoisoindolin-2-yl)acetate (B). A solution of 5-bromo-2,3-dihydroisoindol-1-one (A; 0.5 g, 2.36 mmol, 1.1 equiv.) in THE (5 mL) was added to hexanes washed NaH (0.17 g, 7.074 mmol, 3.0 equiv.) in THE (25 mL) at ice-bath temperature. The mixture was stirred for 30 minutes under argon, tert-butyl 2-bromoacetate (1.38 g, 7.07 mmol, 3.0 equiv.) was then added and the reaction was warmed to room temperature and stirred for 30 minutes. The reaction was quenched with ice-cold water, the two layers were separated, and the aqueous layer was extracted with EtOAc. The combined organic layers were washed with brine, dried over Na₂SO₄ and concentrated to give a white solid. Purification by CombiFlash column chromatography using 100% CH₂Cl₂ to 5% MeOH gave compound B (0.73 g, 2.24 mmol, Yield 94.91%). LC/MS (ESI) m/z: 327 (M+H)⁺.

Step 2: tert-butyl 2-(1-oxo-5-phenoxyisoindolin-2-yl)acetate (C). A mixture of compound B (0.1 g, 0.31 mmol, 1.0 equiv.), phenol (0.043 g, 0.46 mmol, 1.5 equiv.), dimethylaminoaceticacid hydrochloride (0.013 g, 0.092 mmol, 0.3 equiv.), cesium carbonate (0.4 g, 1.23 mmol, 4.0 equiv.), and copper(i) iodide (0.006 g, 1.23 mmol, 0.1 equiv.) in dioxane (5 mL) was heated in a sealed tube overnight. The reaction was cooled to room temperature then diluted with water (5 mL) and EtOAc (5 mL). The two layers were separated, and the aqueous layer was extracted with EtOAc. The combined organic extracts were washed with brine, dried over Na₂SO₄ and concentrated to give a yellow oil. Purification by CombiFlash column chromatography using 100% hexanes to 10% EtOAc gave compound C (0.05 g, 0.15 mmol, 48.06%) as a yellow oil. LC/MS (ESI) m/z: 340 (M+H)⁺.

Step 3: 2-(1-oxo-5-phenoxyisoindolin-2-yl)acetic acid (D). TEA (0.14 mL) was added to a solution of compound 3 (0.06 g, 0.18 mmol, 1.0 equiv.) in CH₂Cl₂ (1 mL) at ice-bath temperature. The reaction was stirred at room temperature for 2 hours and then concentrated to dryness. 3 mL of 3 N HCl was added and the mixture concentrated again to dryness to give compound D (0.05 g, 0.18 mmol, Yield 99.84%) as an off-yellow solid. LC/MS (ESI) m/z: 284 (M+H)⁺.

Step 4: ethyl (1S,3S,5S)-5-methyl-2-(2-(1-oxo-5-phenoxyisoindolin-2-yl)acetyl)-2-azabicyclo[3.1.0]hexane-3-carboxylate (F). To a solution of compound D (0.05 g, 0.18 mmol, 1.0 equiv.) in DMF (2 mL) was added ethyl (1 S,3S,5S)-5-methyl-2-azabicyclo[3.1.0]hexane-3-carboxylate (E; 0.03 g, 0.18 mmol, 1.0 equiv.), HATU (0.087 g, 0.229 mmol, 1.3 equiv.), and DIPEA (0.062 mL, 0.35 mmol, 2.0 equiv.). The reaction was stirred at room temperature for 30 minutes. Water (2 mL) and EtOAc (5 mL) were added. The two layers were separated, and the aqueous layer was extracted with EtOAc, washed with brine, dried over Na₂SO₄, and concentrated. Purification by CombiFlash, 4 g column, solvent A=CH₂Cl₂, solvent B=MeOH, 100% A to 5% B gave compound F (0.058 g, 0.133 mmol, Yield 75.63%) as a yellow oil. LC/MS (ESI) m/z: 435 (M+H)⁺.

Step 5: (1S,3S,5S)-5-methyl-2-(2-(1-oxo-5-phenoxyisoindolin-2-yl)acetyl)-2-azabicyclo-[3.1.0]hexane-3-carboxylic acid (G). To a solution of compound F (0.058, 0.133 mmol, 1.0 equiv.) in MeOH (3 mL) and H₂O (1 mL) was added LiOH (0.006 g, 0.27 mmol, 2.0 equiv.). The reaction was stirred at room temperature overnight, concentrated to remove MeOH, and then diluted with EtOAc (3 mL). The pH was adjusted to 1 using 1 N HCl. The two layers were separated, and the aqueous layer was extracted with EtOAc (3 mL×3). The combined organic extracts were washed with brine, dried over Na₂SO₄ and concentrated to give compound G (0.05 g, Yield 92.15%) as a white solid. LC/MS (ESI) m/z: 407 (M+H)⁺.

Step 6: (1S,3S,5S)-N-((4-carbamimidoylthiophen-2-yl)methyl)-5-methyl-2-(2-(1-oxo-5-phenoxyisoindolin-2-yl)acetyl)-2-azabicyclo[3.1.0]hexane-3-carboxamide (Compound 101). To a solution of compound G (0.05 g, 0.12 mmol, 1.0 equiv.) in DMF (2 mL) was added 5-(aminomethyl)-thiophene-3-carboximidamide (0.028 g, 0.15 mmol, 1.2 equiv.), HATU (0.05 g, 0.14 mmol, 1.1 equiv.), and DIPEA (0.064 mL, 0.35 mmol, 3.0 equiv.). The reaction mixture was stirred at room temperature for 20 minutes and then purified directly by HPLC to give Compound 101 (0.02 g, 0.04 mmol, Yield 29.90%). LC/MS (ESI) m/z: 544 (M+H)⁺. ¹H NMR (400 MHz, DMSO-d₆) δ 8.65 (t, J=5.9 Hz, 1H), 8.40-8.32 (m, 2H), 7.72 (dd, J=8.4, 4.5 Hz, 1H), 7.55-7.43 (m, 2H), 7.29-7.18 (m, 3H), 7.17-7.07 (m, 2H), 4.67 (dd, J=11.4, 3.1 Hz, 1H), 4.64-4.30 (m, 3H), 3.43 (dd, J=6.0, 2.4 Hz, 1H), 2.52 (p, J=1.9 Hz, 2H), 2.31 (t, J=12.4 Hz, 1H), 1.97 (dd, J=13.3, 3.1 Hz, 1H), 1.23 (d, J=10.6 Hz, 4H), 0.65 (t, J=5.5 Hz, 1H).

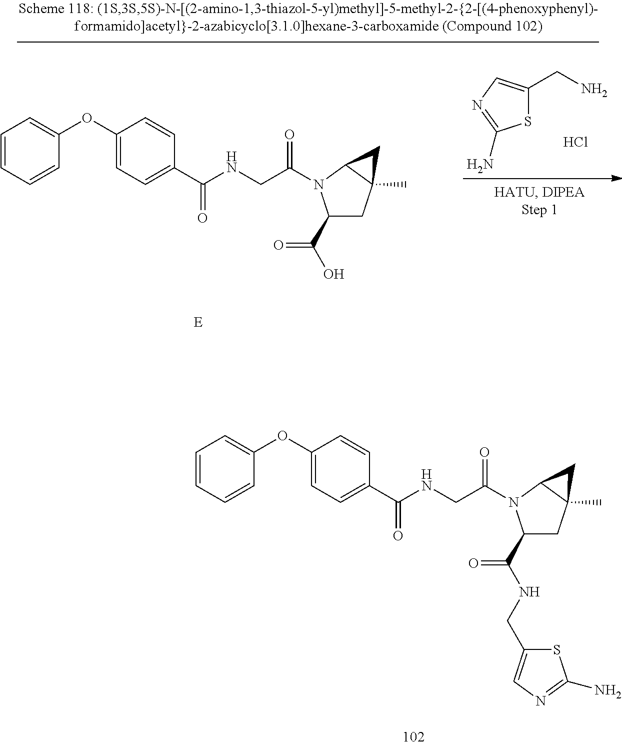 [SMILES: C[C@@]12C[C@@H]1N(C(=O)CNC(=O)c1ccc(Oc3ccccc3)cc1)[C@H](C(=O)O)C2 ; NCc1cnc(N)s1 ; Cl ; C[C@@]12C[C@@H]1N(C(=O)CNC(=O)c1ccc(Oc3ccccc3)cc1)[C@H](C(=O)NCc1cnc(N)s1)C2]

Step 1: (1S,3S,5S)-N-[(2-amino-1,3-thiazol-5-yl)methyl]-5-methyl-2-{2-[(4-phenoxyphenyl)-formamido]acetyl}-2-azabicyclo[3.1.0]hexane-3-carboxamide (Compound 102). To a solution of compound A (0.04 g, 0.10 mmol, 1.0 equiv.) in DMF (2 mL) was added 5-(aminomethyl)thiazol-2-amine (0.02 g, 0.11 mmol, 1.1 equiv.), HATU (0.04 g, 0.11 mmol, 1.1 equiv.), and DIPEA (0.05 mL, 0.30 mmol, 3.0 equiv.). The reaction mixture was stirred at room temperature for 10 minutes and then purified directly by HPLC to give Compound 102 (0.025 g, 0.05 mmol, Yield 48.76%). LC/MS (ESI) m/z: 506 (M+H)⁺. ¹H NMR (400 MHz, DMSO-d₆) δ 8.61 (t, J=5.7 Hz, 1H), 8.25-8.17 (m, 1H), 7.90 (dd, J=8.9, 2.3 Hz, 2H), 7.44 (t, J=7.8 Hz, 2H), 7.21 (t, J=7.4 Hz, 1H), 7.07 (dd, J=21.9, 8.3 Hz, 2H), 6.77 (d, J=2.2 Hz, 1H), 6.73 (s, 1H), 4.60 (dd, J=11.5, 3.0 Hz, 1H), 4.35 (dd, J=16.5, 5.7 Hz, 1H), 4.17 (ddt, J=24.0, 15.2, 7.8 Hz, 2H), 4.01 (dd, J=16.5, 5.5 Hz, 1H), 3.54-3.37 (m, 1H), 2.25 (t, J=12.4 Hz, 1H), 1.94 (dd, J=13.2, 3.0 Hz, 1H), 1.21 (d, J=10.3 Hz, 4H), 0.63-0.66 (m, 1H).

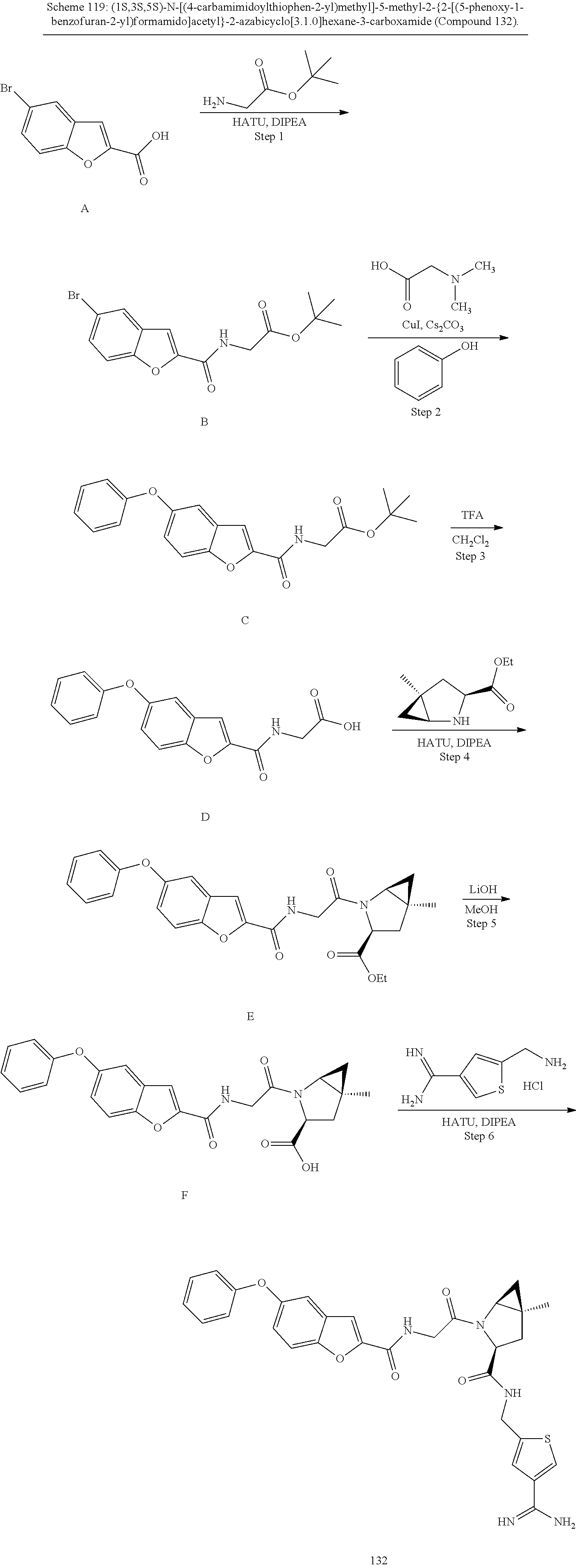

Step 1: tert-butyl 2-[(5-bromo-1-benzofuran-2-yl)formamido]acetate (B). A solution of 5-bromo-1-benzofuran-2-carboxylic acid (A; 1.3 g, 5.39 mmol, 1.0 equiv.) in DMF (5 mL) was added tert-butyl 2-aminoacetate (1.085 g, 6.472 mmol, 1.2 equiv.), HATU (2.67 g, 7.01 mmol, 1.3 equiv.) and DIPEA (2.091 g, 2.826 mL, 16.18 mmol, 3 equiv.). The reaction was stirred at room temperature for 1 h. Water 10 mL was added and the solid collected by filtration and dried to give compound B (1.8 g, 5.08 mmol, Yield 94.23%) as a brown powder. LC/MS (ESI) m/z: 355 (M+H)⁺.

Step 2: tert-butyl 2-[(5-phenoxy-1-benzofuran-2-yl)formamido]acetate (C). A mixture of compound B (0.8 g, 2.26 mmol, 1.0 equiv.), phenol (0.319 g, 3.39 mmol, 1.5 equiv.), dimethylaminoacetic acid hydrochloride (0.095 g, 0.678 mmol, 0.3 equiv.), cesium carbonate (2.94 g, 9.03 mmol, 4.0 equiv.), and copper(I) iodide (0.043 g, 0.226 mmol, 0.1 equiv.) in dioxane (5 mL) was heated in a sealed tube overnight. The reaction was cooled to room temperature then diluted with water (5 mL) and EtOAc (5 mL). The two layers were separated, and the aqueous layer was extracted with EtOAc. The combined organic extracts were washed with brine, dried over Na₂SO₄ and concentrated to give a yellow oil. Purification by CombiFlash column chromatography using 100% hexanes to 20% EtOAc gave compound C (0.5 g, 1.361 mmol, Yield 60.25%) as a yellow oil. LC/MS (ESI) m/z: 368 (M+H)⁺.

Step 3: [(5-phenoxy-1-benzofuran-2-yl)formamido]acetic acid (D). TFA (0.25 mL) was added to a solution of compound C (0.12 g, 0.327 mmol, 1.0 equiv.) in CH₂Cl₂ (1 mL) at ice-bath temperature. The reaction was stirred at room temperature for 2 hours and then concentrated to dryness. 3 mL of 3 N HCl was added and the mixture concentrated again to dryness to give compound D (0.092 g, 0.327 mmol, Yield 90.48%) as an off-yellow solid. LC/MS (ESI) m/z: 312 (M+H)⁺.

Step 4: ethyl (1S,3S,5S)-5-methyl-2-{2-[(5-phenoxy-1-benzofuran-2-yl)formamido]acetyl}-2-azabicyclo[3.1.0]hexane-3-carboxylate (E) To a solution of compound D (0.092 g, 0.295 mmol, 1.0 equiv.) in DMF (1 mL) was added ethyl (1S,3S,5S)-5-methyl-2-azabicyclo[3.1.0]hexane-3-carboxylate (0.05 g, 0.295 mmol, 1.0 equiv.), HATU (0.146 g, 0.384 mmol, 1.3 equiv.) and DIPEA (0.129 mL, 0.739 mmol, 2.5 equiv.). The reaction was stirred at room temperature for 30 minutes. Water (5 mL) and EtOAc (5 mL) were added. The two layers were separated, and the aqueous layer was extracted with EtOAc, washed with brine, dried over Na₂SO₄, and concentrated. Purification by CombiFlash, 12 g column, solvent A=CH₂Cl₂, solvent B=MeOH, 100% A to 5% B gave compound E (0.1 g, 0.216 mmol, Yield 73.18%) as a yellow oil. LC/MS (ESI) m/z: 467 (M+H)⁺.

Step 5: (1S,3S,5S)-5-methyl-2-{2-[(5-phenoxy-1-benzofuran-2-yl)formamido]acetyl}-2-azabicyclo[3.1.0]hexane-3-carboxylic acid (F). To a solution of compound E (0.11, 0.238 mmol, 1.0 equiv.) in MeOH (3 mL) and H₂O (1 mL) was added LiOH (0.011 g, 0.476 mmol, 2.0 equiv.). The reaction was stirred at room temperature overnight, concentrated to remove MeOH, and then diluted with EtOAc (3 mL). The pH was adjusted to 1 using 1 N HCl. The two layers were separated, and the aqueous layer was extracted with EtOAc (3 mL×3). The combined organic extracts were washed with brine, dried over Na₂SO₄ and concentrated to give compound F (0.1 g, 0.23 mmol, Yield 96.78%) as a white solid. LC/MS (ESI) m/z: 435 (M+H)⁺.

Step 6: (1S,3S,5S)-N-[(4-carbamimidoylthiophen-2-yl)methyl]-5-methyl-2-{2-[(5-phenoxy-1-benzofuran-2-yl)formamido]acetyl}-2-azabicyclo[3.1.0]hexane-3-carboxamide (Compound 131). To a solution of compound F (0.09 g, 0.207 mmol, 1.0 equiv.) in DMF (2 mL) was added 5-(aminomethyl)thiophene-3-carboximidamide (0.044 g, 0.228 mmol, 1.1 equiv.), HATU (0.087 g, 0.228 mmol, 1.1 equiv.), and DIPEA (0.145 mL, 0.829 mmol, 4.0 equiv.). The reaction mixture was stirred at room temperature for 30 minutes and then purified directly by HPLC to give Compound 132 (0.01 g, 0.017 mmol, Yield 8.44%). LC/MS (ESI) m/z: 572 (M+H)+. 1H NMR (400 MHz, DMSO-d₆) δ 8.57 (t, J=5.9 Hz, 1H), 8.46 (s, 1H), 8.31 (d, J=9.3 Hz, 1H), 7.71 (d, J=8.9 Hz, 1H), 7.54 (s, 1H), 7.47 (d, J=16.2 Hz, 1H), 7.44-7.35 (m, 2H), 7.24-7.09 (m, 1H), 7.02 (d, J=8.0 Hz, 2H), 4.71-4.63 (m, 1H), 4.50-4.30 (m, 2H), 4.13-3.98 (m, 2H), 3.43 (d, J=5.3 Hz, 1H), 3.17 (s, 1H), 2.28 (d, J=12.2 Hz, 1H), 2.08 (s, 1H), 2.03-1.94 (m, 1H), 1.32-1.11 (m, 3H), 0.85 (dt, J=10.6, 6.7 Hz, 1H), 0.70 (t, J=5.4 Hz, 1H).

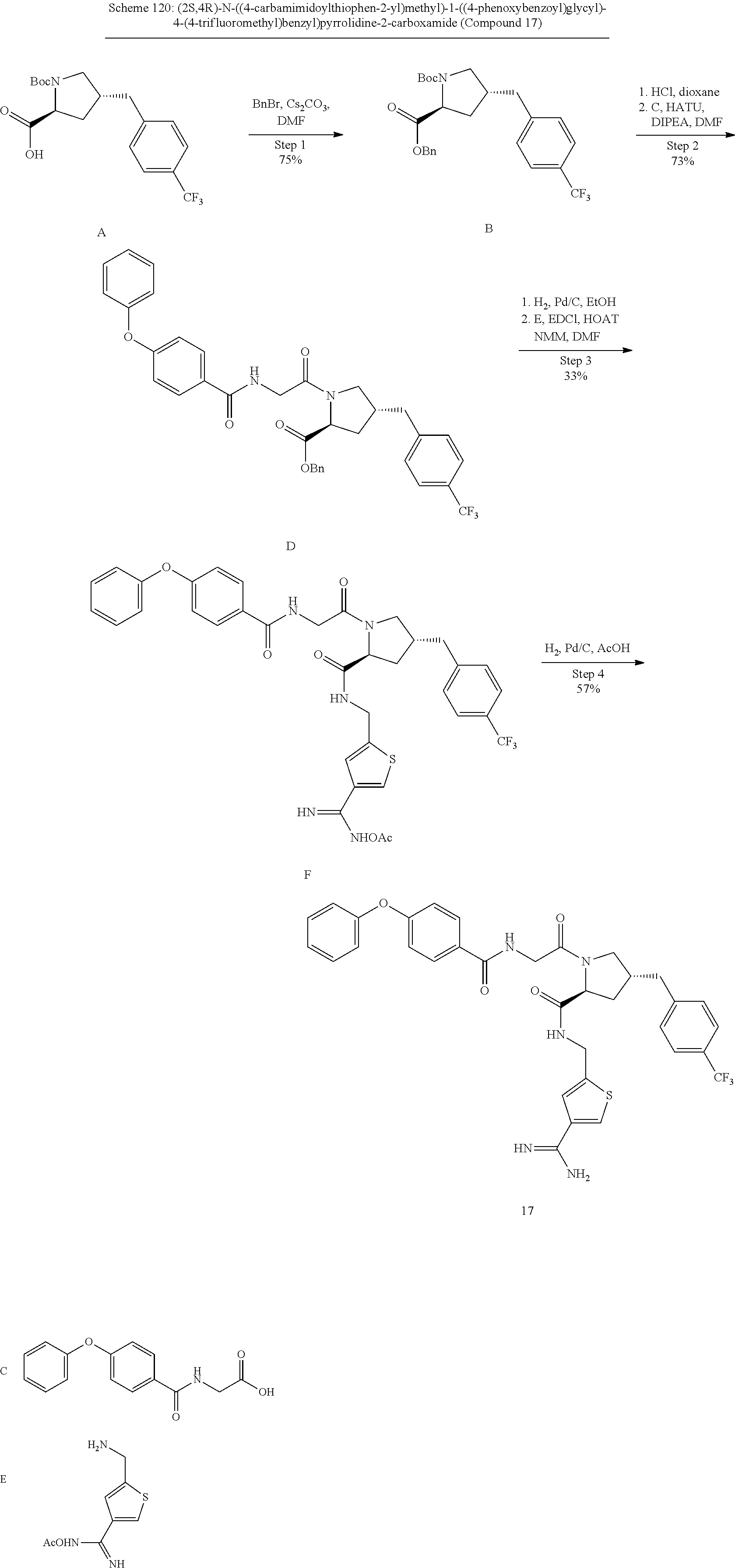

Step 1: 2-Benzyl 1-(tert-butyl) (2S,4R)-4-(4-(trifluoromethyl)benzyl)pyrrolidine-1,2-dicarboxylate (B). To a solution of (2S,4R)-1-(tert-butoxycarbonyl)-4-(4-(trifluoromethyl)benzyl)-pyrrolidine-2-carboxylic acid (A; 0.1 g, 0.268 mmol, 1.0 equiv.) in DMF (1.3 mL) at 0° C. were added Cs₂CO₃ (0.113 g, 0.348 mmol, 1.3 equiv.) followed by benzyl bromide (0.06 g, 0.041 mL, 0.348 mmol). The resulting mixture was stirred at RT for 16 h. The reaction was quenched with the addition of water. The mixture was taken into EtOAc, the aqueous layer was separated and extracted 3 times with EtOAc. The combined organic layers were washed with water 3×, then brine, dried on MgSO₄, filtered and concentrated in vacuo. The residue was purified on silica gel using 0-30% EtOAc/hexanes to give 2-benzyl 1-(tert-butyl) (2S,4R)-4-(4-(trifluoromethyl)benzyl)pyrrolidine-1,2-dicarboxylate (B; 0.094 g, 0.203 mmol, Yield 75.7%) as a colorless oil. HPLC-MS (ESI): m/z [M+Na]⁺: 486. Rt 4.61 min.

Step 2: Benzyl (2S,4R)-1-((4-phenoxybenzoyl)glycyl)-4-(4-(trifluoromethyl)benzyl)-pyrrolidine-2-carboxylate (D). To a vial containing 2-benzyl 1-(tert-butyl) (2S,4R)-4-(4-(trifluoromethyl)benzyl) pyrrolidine-1,2-dicarboxylate (B; 0.038 g, 0.082 mmol, 1 equiv.) was added 4 N HCl in dioxane (0.41 mL, 1.64 mmol, 20 equiv.) at room temperature and the resulting mixture was stirred for 3 hr. The volatiles were then removed in vacuo to afford benzyl (2S,4R)-4-(4-(trifluoromethyl)benzyl)-pyrrolidine-2-carboxylate hydrochloride (0.033 g, 0.083 mmol, Yield 100.7%) as a colorless oil. HPLC-MS (ESI): m/z [M+H]⁺: 364. Rt 2.7 min.

To a solution of (4-phenoxybenzoyl)glycine C (0.027 g, 0.098 mmol, 1.2 equiv.) and benzyl (2S,4R)-4-(4-(trifluoromethyl)benzyl)pyrrolidine-2-carboxylate hydrochloride (0.033 g, 0.082 mmol, 1 equiv.) in DMF (0.41 mL) at 0° C. was added HATU (0.047 g, 0.123 mmol, 1.5 equiv.) followed by DIPEA (0.071 mL, 0.41 mmol, 5 equiv.) and the resulting mixture was stirred at 0° C. for 45 minutes. The reaction was quenched with the addition of water. The mixture was taken into EtOAc, the aqueous layer separated, and two more extractions with water were done to remove the DMF. The combined aqueous layers were back extracted with Et₂O. The combined organic layers were washed with brine, dried on MgSO₄, filtered and concentrated in vacuo. The crude material was suspended on silica gel and purified using 0-100% EtOAc/hexanes to yield benzyl (2S,4R)-1-((4-phenoxybenzoyl)glycyl)-4-(4-(trifluoromethyl)benzyl) pyrrolidine-2-carboxylate (D; 0.037 g, 0.06 mmol, Yield 73.2%) as a colorless oil. HPLC-MS (ESI): m/z [M+H]⁺: 617. Rt 4.4 min.

Step 3: ((2S,4R)-N-((4-(N-acetoxycarbamimidoyl)thiophen-2-yl)methyl)-1-((4-phenoxy-benzoyl)glycyl)-4-(4-(trifluoromethyl)benzyl)pyrrolidine-2-carboxamide (F). A solution of benzyl (2S,4R)-1-((4-phenoxybenzoyl)glycyl)-4-(4-(trifluoromethyl)benzyl)pyrrolidine-2-carboxylate (D; 0.037 g, 0.06 mmol, 1 equiv.) in ethanol (1 mL) was degassed by bubbling with argon. Pd/C (0.006 g, 0.006 mmol, 0.1 equiv.) was added and the solution degassed by bubbling argon once more. Then a balloon of H₂ was added, bubbling directly into the solution for 30 seconds. The resulting suspension was stirred at RT for 1 h. The mixture was degassed with nitrogen, diluted with DCM, celite was added and the mixture was filtered on a pad of celite, rinsed with DCM, then rinsed with MeOH. The volatiles were removed in vacuo to give (2S,4R)-1-((4-phenoxybenzoyl)glycyl)-4-(4-(trifluoromethyl)benzyl) pyrrolidine-2-carboxylic acid (0.035 g, 0.066 mmol, Yield 110.8%) as a colorless solid, which was used as is in the next step. HPLC-MS (ESI): m/z [M+H]⁺:527. Rt 3.7 min.

To a solution of (2S,4R)-1-((4-phenoxybenzoyl)glycyl)-4-(4-(trifluoromethyl)benzyl)pyrrolidine-2-carboxylic acid (0.035 g, 0.066 mmol, 1.0 equiv.) in DMF (1.5 mL) cooled in an ice/NaCl bath was added EDCI hydrochloride (0.016 g, 0.081 mmol, 1.2 equiv.) followed by HOAT (0.009 g, 0.068 mmol, 1.0 equiv.). The reaction was stirred for 30 mins. 5-(aminomethyl)thiophene-3-carboximidamide dihydrochloride (E; 0.019 g, 0.068 mmol, 1.0 equiv.) was added followed by dropwise addition of 4-methylmorpholine (0.022 mL, 0.203 mmol, 3.0 equiv.). The reaction was stirred for 20 hrs and allowed to warm to room temperature. The reaction was then concentrated and partitioned between solutions of 3% DCM/MeOH solution and NaHCO₃ (sat). The organic layer was separated and the aqueous layer extracted 3× with 3% DCM/MeOH. The combined organic layers were washed with brine, dried over Na₂SO₄ filtered and concentrated in vacuo. The crude material was suspended on silica gel and purified on a Redisep gold column (12 g) using a gradient of 15 to 70%; A=Ethyl acetate/B=20% MeOH in EtOAc with 15% B at t=0. Impure fractions were pooled and re-purified using 10 to 50% of the same solvent system with 10% B at t=0 to provide (2S,4R)-N-((4-(N-acetoxycarbamimidoyl)thiophen-2-yl)methyl)-1-((4-phenoxybenzoyl)glycyl)-4-(4-(trifluoromethyl)benzyl)pyrrolidine-2-carboxamide (F; 16 mg, 0.022 mmol, Yield 32.8%). HPLC-MS (ESI): m/z [M+H]⁺: 722.3, Rt=3.74 min.

Step 4: (2S,4R)-N-((4-carbamimidoylthiophen-2-yl)methyl)-1-((4-phenoxybenzoyl)glycyl)-4-(4-(trifluoromethyl)benzyl)pyrrolidine-2-carboxamide (Compound 17). A flask containing (2S,4R)-N-((4-(N-acetoxycarbamimidoyl)thiophen-2-yl)methyl)-1-((4-phenoxybenzoyl) glycyl)-4-(4-(trifluoromethyl)-benzyl)pyrrolidine-2-carboxamide (F; 16.7 mg, 0.023 mmol, 1.0 equiv.) was evacuated and placed under argon followed by the addition of acetic acid (1 mL). Argon was bubbled into the solution for 10 mins. Pd/C 10% (0.002 g, 0.002 mmol, 0.1 equiv.) was then added and argon was bubbled for 5 mins. H₂ was added into the reaction for 5 mins, after which the reaction was allowed to react under a H₂ atmosphere for 20 hrs. The reaction was concentrated, MeOH was added, and the mixture was filtered through a 45 um teflon syringe filter and concentrated again. MeOH (0.5 ml) was added and the solution was cooled to 0° C. 4 N HCl in dioxane (0.023 mL, 0.093 mmol, 4 equiv.) was added and the reaction was stirred for 2 hours, after which the reaction mixture was concentrated and left under high vacuum for 3 hours. The residue was then suspended in EtOAc and stirred for 16 hrs. The suspension was collected by filtration and dried under high vacuum to provide Compound 17 (9.2 mg, 0.013 mmol, 57.1%). Purity 96.2%, Rt=9.82 min) as a beige solid. ¹H NMR (400 MHz, CD₃OD): δ 8.23 (1H, d, J=1.7 Hz), 8.19 (1H, d, J=1.6 Hz), 7.83 (2H, d, J=8.5 Hz), 7.58-7.62 (2H, m), 7.37-7.47 (5H, m), 7.21 (1H, t, J=7.4 Hz), 7.06 (2H, d, J=8.0 Hz), 7.00 (2H, d, J=8.4 Hz), 4.55-4.59 (3H, m), 4.10-4.21 (2H, m), 3.85 (1H, dd, J=9.9, 7.5 Hz), 3.40 (1H, t, J=9.4 Hz), 2.69-2.90 (3H, m), 1.99-2.14 (2H, m). HPLC-MS (ESI): m/z [M+H]⁺: 664.3, Rt=2.99 min.

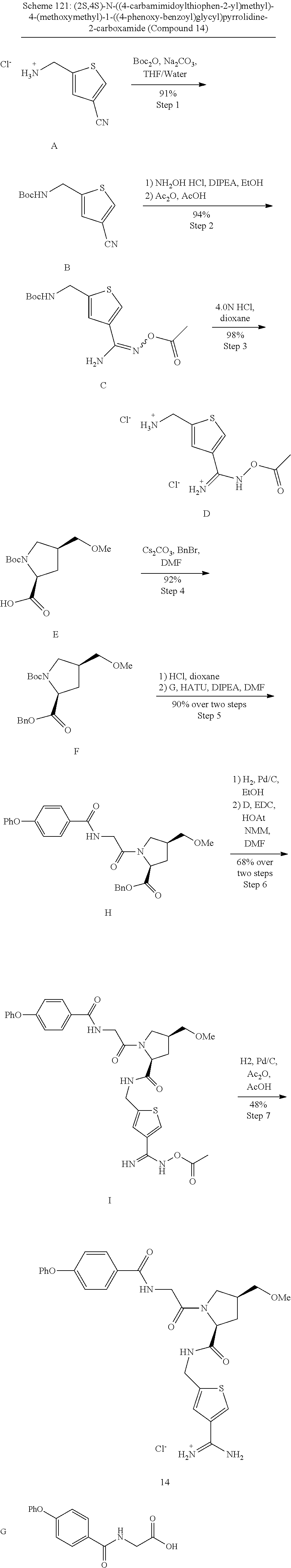

Step 1: tert-butyl ((4-cyanothiophen-2-yl)methyl)carbamate (B). Into a 100 mL round-bottomed flask equipped with a Teflon-coated magnetic stirring bar was added 5-(aminomethyl)-thiophene-3-carbonitrile hydrochloride (A; 1.97 g, 11.3 mmol, 1.00 equiv), THE (20 mL) and water (7 mL). The resulting solution was stirred at 0° C. in an ice bath. Sodium carbonate (1.20 g, 11.3 mmol, 1.00 equiv) was added in one portion and the reaction mixture was continued to stir at 0° C. for 5 min. Di-tert-butyl-dicarbonate (5.43 g, 22.6 mmol, 2.00 equiv) was added to a scintillation vial and dissolved in tetrahydrofuran (4 mL). The resulting solution was taken up into a syringe and added dropwise to the reaction mixture at 0° C. The reaction mixture was stirred at 0° C., gradually warming to RT over a period of 48 h. The reaction mixture was concentrated in vacuo and diluted with water. The aqueous solution was extracted with EtOAc (3×). The combined extracts were washed sequentially with 1 N HCl (aq), 1 M NaOH (aq), and brine. The organic layer was dried with Na₂SO₄, filtered and concentrated in vacuo. The crude product was purified by silica gel column chromatography using a gradient of 10-50% EtOAc in hexanes to afford compound B (2.44 g, 10.2 mmol, 90.5% yield) as a white solid. Rr (30% EtOAc in Hexanes)=0.34; ¹H NMR (CDCl₃, 400 MHz): δ 7.81 (1H, s), 7.11 (1H, s), 5.01 (1H, br s), 4.45 (2H, d, J=6.2 Hz), 1.46 (9H, s); LC/MS (ESI+) m/z 240.1 [M+H]⁺, 260.1 [M+Na]⁺, Rt=2.97 min.

Step 2: tert-butyl ((4-(N′-acetoxycarbamimidoyl)thiophen-2-yl)methyl)carbamate (C). Into a 250 mL round-bottomed flask equipped with a Teflon-coated magnetic stirring bar was added compound B (2.43 g, 10.2 mmol, 1.00 equiv) and anhydrous EtOH (50 mL). The resulting solution was stirred at 0° C. in an ice bath. Hydroxylamine hydrochloride (1.77 g, 25.5 mmol, 2.50 equiv.) was added in one portion followed by dropwise addition of DIPEA (5.34 mL, 30.6 mmol, 3.00 equiv.) over 20 min. The ice bath was removed and the reaction mixture stirred at RT for 16 h. The reaction mixture was diluted with EtOAc and washed with water (×2). The combined aqueous washes were back-extracted with EtOAc (×3). The combined organic layers were washed with brine, dried with Na₂SO₄, filtered and concentrated in vacuo to afford tert-butyl ((4-(N′-hydroxycarbamimidoyl)thiophen-2-yl)methyl) (2.72 g, 10.0 mmol, 98.3% yield) as a white solid that was used without further purification. LC/MS (ESI+) m/z 272.1 [M+H]⁺, 294.1 [M+Na]⁺, Rt=1.20 min.

Into a 100 mL round-bottomed flask equipped with a Teflon-coated magnetic stirring bar was added tert-butyl ((4-(N′-hydroxycarbamimidoyl)thiophen-2-yl)methyl) (2.71 g, 10.0 mmol, 1.00 equiv.) and acetic acid (20 mL). Acetic anhydride (3.40 mL, 36.0 mmol, 3.6 equiv.) was added and the resulting solution was stirred at RT for 1 h. The reaction mixture was concentrated in vacuo and diluted with EtOAc. The solution was washed with sat. NaHCO₃ (aq), which was back-extracted with EtOAc (×2). The combined organic layers were washed with brine, dried with Na₂SO₄, filtered and concentrated in vacuo. The crude product was purified through a combination of recrystallization from EtOAc/hexanes and subsequent silica gel column chromatography of the mother liquor using a gradient of 50-70% EtOAc in hexanes to afford compound C (2.94 g, 9.4 mmol, 93.9% yield) as a white solid. Rr(70% EtOAc in Hexanes)=0.32; ¹H NMR (DMSO-d6, 400 MHz): δ 7.86 (1H, s), 7.52 (1H, s), 7.19 (1H, s), 6.68 (2H, s), 4.24 (2H, d, J=6.1 Hz), 2.12 (3H, s), 1.39 (9H, s); LC/MS (ESI+) m/z 314.1 [M+H], 337.1 [M+Na]⁺, Rt=2.53 min.

Step 3: N-acetoxy-5-(aminomethyl)thiophene-3-carboximidamide dihydrochloride (D). Into a 100 mL round-bottomed flask equipped with a Teflon-coated magnetic stirring bar was added compound C (1.87 g, 5.97 mmol, 1.00 equiv.). The flask was sealed and cooled in an ice bath. Hydrogen chloride (4.0 M in dioxane, 29.2 mL, 117 mmol, 19.6 equiv.) was added and the reaction mixture was stirred at 0° C., gradually warming to RT over 16 h. The reaction mixture was concentrated in vacuo and the crude product was suspended in EtOAc with vigorous stirring at RT for 5 min. The suspension was filtered, and the precipitate was washed with additional EtOAc and dried in vacuo to afford compound D (1.70 g, 5.94 mmol, 99.5% yield) as a white solid (bis-HCl salt) that was used without further purification. ¹H NMR (DMSO-d6, 400 MHz): δ 8.55 (3H, br s), 8.13 (1H, s), 7.55 (1H, s), 6.92 (1H, br s), 4.21 (2H, s), 2.13 (3H, s). LC/MS (ESI+) m/z 214.1 [M+H]⁺, Rt=0.26 min.

Step 4: 2-Benzyl 1-(tert-butyl) (2S,4S)-4-(methoxymethyl)pyrrolidine-1,2-dicarboxylate (F). Into a 10 mL round-bottomed flask equipped with a Teflon-coated magnetic stirring bar was added compound E (175 mg, 0.675 mmol, 1.00 equiv.) and anhydrous DMF (1.2 mL). The resulting solution was stirred at 0° C. in an ice bath. Cesium carbonate (286 mg, 0.877 mmol, 1.30 equiv.) was added in one portion and the resulting solution was stirred at 0° C. for 5 min. Benzyl bromide (0.10 mL, 0.877 mmol, 1.30 equiv.) was added and the reaction mixture was stirred at 0° C. for 30 min and at RT for 3 h. The reaction mixture was diluted with EtOAc and filtered over celite, washing with additional EtOAc. The filtrate was concentrated in vacuo onto silica gel. The crude mixture was purified by silica gel column chromatography using a gradient of 0-40% EtOAc/hexanes to afford compound F (218 mg, 0.624 mmol, 92.4% yield) as a colorless oil. LC/MS (ESI+) m/z 372.2 [M+Na]⁺, Rt=3.72 min.

Step 5: Benzyl (2S,4S)-4-(methoxymethyl)-1-((4-phenoxybenzoyl)glycyl)pyrrolidine-2-carboxylate (H). Into a 25 mL round-bottomed flask equipped with a Teflon-coated magnetic stirring bar was added compound F (132 mg, 0.378 mmol, 1.00 equiv) and anhydrous dioxane (0.6 mL). The resulting solution was stirred at 0° C. in an ice bath. Hydrogen chloride (4.0 M in dioxane, 2.36 mL, 9.44 mmol, 25.0 equiv) was added and the resulting solution was stirred at RT for 3 h. The volatiles were removed in vacuo to afford benzyl (2S,4S)-4-(methoxymethyl)pyrrolidine-2-carboxylate hydrochloride (110 mg, 0.385 mmol, 101% yield) as a colorless oil that was used without further purification. LC/MS (ESI+) m/z 250.2 [M+H]⁺, Rt=1.21 min.

Into a 25 mL round-bottomed flask equipped with a Teflon-coated magnetic stirring bar was added compound F (123 mg, 0.454 mmol, 1.20 equiv.), HATU (216 mg, 0.567 mmol, 1.50 equiv.) and anhydrous DMF (1.0 mL). The resulting solution was stirred at 0° C. in an ice bath. DIPEA (79.0 μL, 0.454 mmol, 1.20 equiv) was added drop-wise and the reaction mixture was stirred at 0° C. for 15 min. (4-phenoxybenzoyl)glycine (108 mg, 0.378 mmol, 1.00 equiv.) dissolved in anhydrous DMF (1.0 mL) was added to the reaction solution followed by dropwise addition of DIPEA (0.25 mL, 1.44 mmol, 3.80 equiv.). The reaction mixture was stirred at 0° C. for 1 h, quenched with 2 M HCl (aq) (0.1 mL) and diluted with EtOAc. The solution was washed sequentially with 10% citric acid (aq), sat. NaHCO₃ (aq) and brine (×3). The organic phase was dried with Na₂SO₄, filtered and concentrated in vacuo. The crude product was purified by silica gel column chromatography using a gradient of 0-100% EtOAc in hexanes to afford compound H (171 mg, 0.34 mmol, 90.0% yield) as a pale-yellow oil. Rr(80% EtOAc in hexanes)=0.29; LC/MS (ESI+) m/z 503.2 [M+H]⁺, 525.2 [M+Na]⁺, Rt=3.71 min.

Step 6: (2S,4S)-N-((4-(N-acetoxycarbamimidoyl)thiophen-2-yl)methyl)-4-(methoxymethyl)-1-((4-phenoxybenzoyl)glycyl)pyrrolidine-2-carboxamide (1). Into a 25 mL round-bottomed flask equipped with a Teflon-coated magnetic stirring bar was added compound H (170 mg, 0.338 mmol, 1.00 equiv.) and anhydrous EtOH (3.3 mL). The resulting solution was stirred at RT and degassed with N₂. 10% Pd/C (36 mg) was added and the reaction mixture was again degassed with N₂. The reaction mixture was then purged with and stirred under an atmosphere of H₂ at RT for 2 h. The reaction mixture was degassed with N₂, diluted with a 1:1 CHCl₃:EtOH solution and filtered over a short pad of celite, rinsing with additional CHCl₃:EtOH. The filtrate was concentrated in vacuo to afford compound (2S,4S)-4-(methoxymethyl)-1-((4-phenoxybenzoyl)glycyl)pyrrolidine-2-carboxylic acid (140 mg, 0.338 mmol, quantitative) as a colorless oil that was used without further purification. LC/MS (ESI+) m/z 413.1 [M+H]⁺, Rt=2.78 min.

Into a 25 mL round-bottomed flask equipped with a Teflon-coated magnetic stirring bar was added compound 8.1 (83.0 mg, 0.201 mmol, 1.00 equiv.), HOAt (27.0 mg, 0.201 mmol, 1.00 equiv) and anhydrous DMF (2.0 mL). The resulting solution was stirred at −5° C. in an ice-brine bath. EDC (46.3 mg, 0.241 mmol, 1.20 equiv) was added and the reaction mixture was stirred at −5° C. for 30 min. Compound D (60.5 mg, 0.211 mmol, 1.05 equiv) and NMM (44.0 μL, 0.402 mmol, 2.00 equiv) were added and the reaction mixture was stirred at −5° C., gradually warming to RT over 16 h. 10% citric acid (aq) was added and the reaction mixture was extracted with EtOAc (×5). The combined organic extracts were washed with brine, dried with Na₂SO₄, filtered and concentrated in vacuo. The crude product was purified by two rounds of silica gel chromatography, first using a gradient of 3-10% MeOH in CH₂Cl₂ and second with a gradient of 5-10% MeOH in EtOAc to afford compound I (83.0 mg, 0.137 mmol, 67.8% yield) as a white foam. Rr(10% MeOH in CH₂Cl₂)=0.42; HPLC (254 nm) purity 97.9%, Rt=10.64 min; LC/MS (ESI+) m/z 608.2 [M+H]⁺, 631.2 [M+Na]⁺, Rt=2.90 min.

Step 7: (2S,4S)-N-((4-carbamimidoylthiophen-2-yl)methyl)-4-(methoxymethyl)-1-((4-phenoxybenzoyl)glycyl)pyrrolidine-2-carboxamide hydrochloride (Compound 14). Into a 10 mL round-bottomed flask equipped with a Teflon-coated magnetic stirring bar was added compound I (22.0 mg, 0.036 mmol, 1.00 equiv.), acetic acid (0.5 mL) and acetic anhydride (7.0 μL, 0.072 mmol, 2.00 equiv.). The resulting solution was stirred at RT for 10 min. 10% Pd/C (4.0 mg) was added and the reaction mixture was degassed with N₂. The reaction mixture was then purged with and stirred under an atmosphere of H₂ at RT for 14 h. The reaction mixture was degassed with N₂, diluted with acetic acid (0.5 mL) and filtered using a syringe filter (0.45 μm). The filtrate was concentrated in vacuo to yield crude Compound 14 as an amber syrup. The crude product was suspended in EtOAc (5.0 mL) and treated with hydrogen chloride (4.0 M in Dioxane, 1.0 mL) with vigorous stirring and sonication. The suspension was pelleted by centrifugation and carefully decanted. The pellet was washed with additional EtOAc (×2) and the combined supernatants were concentrated by 50% vol. in vacuo to induce precipitation. The precipitate was isolated by centrifugation/decantation and dried in vacuo to give Compound 14 (7.2 mg, 0.012 mmol, 34.2% yield). ¹H NMR (CD₃OD, 400 MHz): δ 8.21 (1H, s), 7.85 (2H, d, J=8.4 Hz), 7.40-7.43 (3H, m), 7.21 (1H, t, J=7.4 Hz), 7.07 (2H, d, J=8.0 Hz), 7.01 (2H, d, J=8.4 Hz), 4.58 (2H, s), 4.45 (1H, t, J=8.2 Hz), 4.09-4.25 (2H, m), 3.95 (1H, t, J=8.9 Hz), 3.40-3.47 (3H, m), 3.34 (3H, s), 2.65 (1H, s), 2.41-2.46 (1H, m), 1.76-1.81 (1H, m); HPLC (254 nm) purity 95.0%, Rt=8.84 min; LC/MS (ESI+) m/z 550.2 [M+H]⁺, 572.2 [M+Na]⁺, Rt=2.14 min.

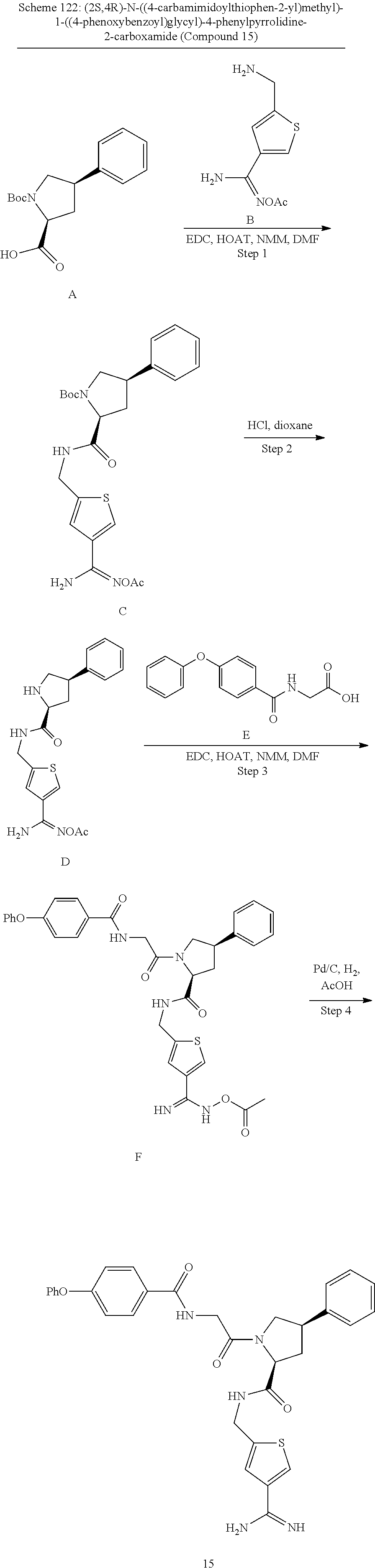

Step 1: tert-butyl (2S,4R)-2-(((4-((E)-N′-acetoxycarbamimidoyl)thiophen-2-yl)methyl)carbamoyl)-4-phenylpyrrolidine-1-carboxylate (C). In a 5 mL vial was introduced (2S,4R)-1-(tert-butoxycarbonyl)-4-phenylpyrrolidine-2-carboxylic acid (A; 40 mg, 0.137 mmol, 1 equiv.), (E)-N′-acetoxy-5-(aminomethyl)thiophene-3-carboximidamide hydrochloride (B; 0.039 g, 0.137 mmol, 1 equiv.), HOAT (0.028 g, 0.206 mmol, 1.5 equiv.) and EDC-HCl (0.039 g, 0.206 mmol, 1.5 equiv.), which were dissolved in DMF (393.258 μL, 0.4 M, 9.831 Vols). The reaction was cooled down to 0° C. and 4-methylmorpholine (0.069 g, 0.075 mL, 0.686 mmol, 5 equiv.) was added dropwise. The reaction was left to stir at room temperature for an hour, upon which LC-MS analysis showed complete conversion of the starting material. The mixture was extracted with ethyl acetate three times. The organic layers were gathered and washed with water and brine. The organic phase was dried with sodium sulfate and solvent was evaporated in vacuo to obtain tert-butyl (2S,4R)-2-(((4-((E)-N′-acetoxycarbamimidoyl)thiophen-2-yl)methyl)carbamoyl)-4-phenylpyrrolidine-1-carboxylate, which was used without further purification.

Step 2: (2S,4R)-N-((4-((E)-N′-Acetoxycarbamimidoyl)thiophen-2-yl)methyl)-4-phenyl-pyrrolidine-2-carboxamide dihydrochloride (D). In a 25 mL round bottom flask was introduced tert-butyl (2S,4R)-2-(((4-((E)-N′-acetoxycarbamimidoyl)thiophen-2-yl)methyl)carbamoyl)-4-phenylpyrrolidine-1-carboxylate (71 mg, 0.146 mmol, 1 equiv.), which was dissolved in DCM (10 mL, 0.016 M, 141 Vols). HCl (4 M) in dioxane (0.73 mmol, 5 equiv.) was added dropwise to the stirring mixture and left to stir until LC-MS analysis showed complete disappearance of the starting material. HCl was evaporated under reduced pressure at room temperature and then solvent was removed using a rotary evaporator. (2S,4R)-N-((4-((E)-N′-Acetoxycarbamimidoyl)thiophen-2-yl)methyl)-4-phenylpyrrolidine-2-carboxamide dihydrochloride was obtained as a white solid and used without further purification.

Step 3: (2S,4R)-N-((4-((E)-N′-acetoxycarbamimidoyl)thiophen-2-yl)methyl)-1-((4-phenoxybenzoyl)glycyl)-4-phenylpyrrolidine-2-carboxamide (F). In a 5 mL vial was introduced (4-phenoxybenzoyl)glycine (E; 17.715 mg, 0.065 mmol, 1 equiv.), EDC-HCl (18.778 mg, 0.098 mmol, 1.5 equiv.), (2S,4R)-N-((4-((E)-N′-Acetoxycarbamimidoyl)thiophen-2-yl)methyl)-4-phenylpyrrolidine-2-carboxamide dihydrochloride (30 mg, 0.065 mmol, 1 equiv.), and HOAT (0.013 g, 0.098 mmol, 1.5 equiv.), which were dissolved in DMF (0.5 mL, 0.131 M, 28.224 Vols). The reaction was cooled down to 0° C. and NMM (33.027 mg, 0.036 mL, 0.327 mmol, 5 equiv.) was added dropwise. The reaction was left to stir at room temperature until LC-MS analysis showed complete conversion of the starting material. The mixture was evaporated under reduced pressure, re-dissolved in DCM, and purified by flash chromatography using DCM/(DCM/MeOH 9:1 solution) as the eluant. (2S,4R)-N-((4-((E)-N′-Acetoxycarbamimidoyl)thiophen-2-yl)methyl)-1-((4-phenoxybenzoyl)glycyl)-4-phenylpyrrolidine-2-carboxamide (F; 25 mg, 0.039 mmol, Yield 59.842%) was obtained as a white solid.

Step 4: (2S,4R)-N-((4-carbamimidoylthiophen-2-yl)methyl)-1-((4-phenoxybenzoyl)glycyl)-4-phenylpyrrolidine-2-carboxamide (Compound 15). In a 25 mL round bottom flask, was dissolved (2S,4R)-N-((4-((E)-N′-acetoxycarbamimidoyl)thiophen-2-yl)methyl)-1-((4-phenoxybenzoyl)glycyl)-4-phenylpyrrolidine-2-carboxamide (18 mg, 0.028 mmol, 1 equiv.) in acetic acid (15 mL, 9.379e−4 M). The reaction was purged with nitrogen and Pd/C (10%) (0.014 mmol, 0.5 equiv.) was added to the stirring mixture. Hydrogen was bubbled into the reaction and the reaction was stirred until the LC-MS analysis showed complete conversion of the starting material. The reaction was then purged with nitrogen, diluted with 10 mL of isopropanol and filtered over a pad of celite. The crude mixture was triturated in ethyl acetate until a white precipitate crashed out. The precipitate was filtered over fritted glass filter and finally re-dissolved in isopropanol. Solvent was removed in vacuo to afford Compound 15 (15 mg, 0.023 mmol, Yield 83%) as an off-white solid. ¹H NMR (400 MHz, CD₃OD): δ_{H} ¹H NMR (CH₃₀H-d₄, 400 MHz): δ_{H} 8.54 (2H, s), 8.20 (1H, s), 7.85 (3H, d, J=8.5 Hz), 7.44-7.40 (4H, m), 7.34 (5H, d, J=4.3 Hz), 7.28-7.25 (1H, m), 7.21 (1H, s), 3.59-3.52 (1H, m), 4.29 (1H, s), 4.53 (2H, t, J=8.8 Hz), 7.03 (5H, dd, J=23.4, 8.2 Hz), 4.60 (3H, s), 4.19 (1H, s), 3.67 (1H, t, J=10.4 Hz), 2.73-2.65 (1H, m), 2.06 (1H, q, J=11.4 Hz). Purity: 93.5%, Rt=8.91 min. HPLC-MS (ESI): m/z [M+H]⁺: 582.3, Rt=2.61 min.

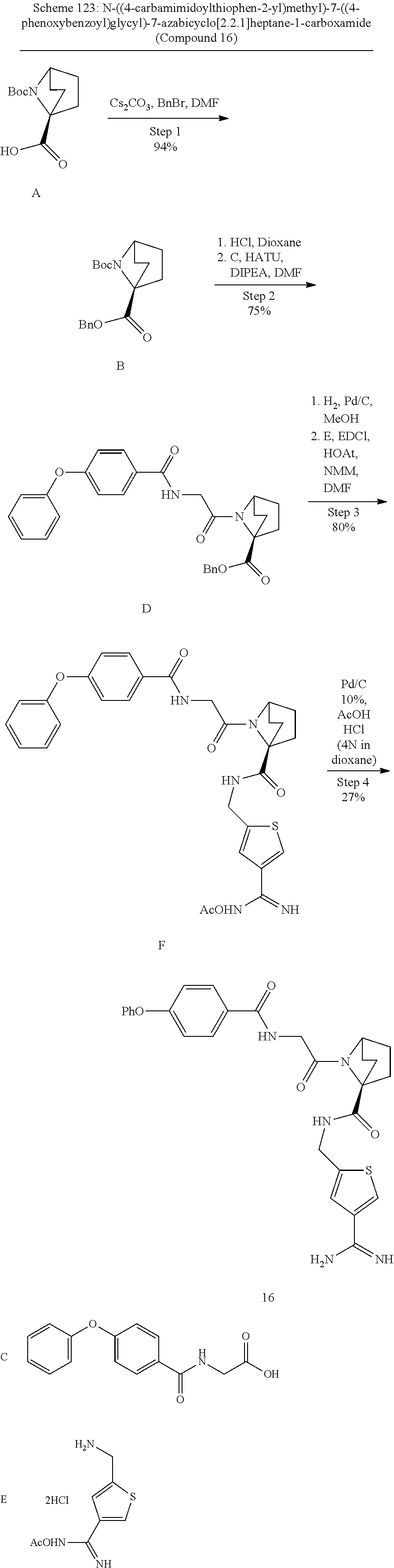

Step 1: 1-benzyl 7-(tert-butyl) 7-azabicyclo[2.2.1]heptane-1,7-dicarboxylate (B). To a vial containing (7-(tert-butoxycarbonyl)-7-azabicyclo[2.2.1]heptane-1-carboxylic acid (A; 0.15 g, 0.622 mmol, 1 equiv.) and Cs₂CO₃ (0.263 g, 0.808 mmol, 1.3 equiv.) was added DMF (1.5 mL). The suspension was cooled to 0° C. before adding benzyl bromide (0.096 mL, 0.808 mmol, 1.3 equiv.) and the resulting heterogeneous mixture was stirred for 2 h. The reaction was diluted with EtOAc, celite was added and the mixture filtered on celite. The residue was suspended on silica gel and purified using 0-25% EtOAc/hexanes to give 1-benzyl 7-tert-butyl 7-azabicyclo[2.2.1]heptane-1,7-dicarboxylate (B; 0.194 g, 0.585 mmol, yield 94.2%) as a colorless oil that crystallized into a colorless solid. HPLC-MS (ESI): m/z [M+Na]⁺: 354. Rt=3.95 min.

Step 2: Benzyl 7-((4-phenoxybenzoyl)glycyl)-7-azabicyclo[2.2.1]heptane-1-carboxylate (D). To a solution of 1-benzyl 7-tert-butyl 7-azabicyclo[2.2.1]heptane-1,7-dicarboxylate (B; 0.099 g, 0.3 mmol, 1 equiv.) in dioxane (0.298 mL) at 0° C. was added 4N HCl in Dioxane (1.5 mL, 6 mmol, 20 equiv.) and the resulting mixture was stirred for 3 h at RT. The volatiles were then removed in vacuo to afford benzyl 7-azabicyclo[2.2.1]heptane-1-carboxylate hydrochloride (0.081 g, 0.303 mmol, Yield 100.84%) as a colorless oil. HPLC-MS (ESI): m/z [M+H]⁺: 232. Rt=0.54 min.

To a solution of (4-phenoxybenzoyl)glycine (C; 0.065 g, 0.24 mmol, 1.2 equiv.) and benzyl 7-azabicyclo[2.2.1]heptane-1-carboxylate hydrochloride (0.054 g, 0.2 mmol, 1 equiv.) in DMF (0.35 mL) at 0° C. were added HATU (0.114 g, 0.3 mmol, 1.5 equiv.) followed by DiPEA (0.129 g, 0.174 mL, 1 mmol, 5 equiv.). The resulting mixture was stirred at 0° C. for 45 minutes. The reaction was quenched with the addition of water. The mixture was taken in EtOAc, the aqueous layer separated and 2 more extractions with water were done to remove the DMF. The combined aqueous layers were back extracted with Et₂O. The combined organic layers were washed with brine, dried on MgSO₄, filtered and concentrated in vacuo. The crude material was suspended on silica gel and purified by using 0-100% EtOAc/hexanes to provide benzyl 7-((4-phenoxybenzoyl)glycyl)-7-azabicyclo[2.2.1]heptane-1-carboxylate (D; 0.073 g, 0.151 mmol, Yield 75.3%) as a yellow oil. HPLC-MS (ESI): m/z [M+H]⁺: 485. Rt=3.95 min.

Step 3. N-((4-(N-acetoxycarbamimidoyl)thiophen-2-yl)methyl)-7-((4-phenoxybenzoyl)-glycyl)-7-azabicyclo[2.2.1]heptane-1-carboxamide (F). A solution of benzyl 7-((4-phenoxybenzoyl)-glycyl)-7-azabicyclo[2.2.1]heptane-1-carboxylate (D; 0.073 g, 0.151 mmol, 1 equiv.) in ethanol (2.5 mL) was degassed with argon. Then Pd/C (0.016 g, 0.015 mmol, 0.1 equiv.) was added and the solution degassed once more. Then a balloon of H₂ was added, bubbling directly in the solution for 30 seconds. The resulting suspension was stirred at RT for 1 h. The mixture was degassed with nitrogen, diluted with DCM, celite was added and the mixture was filtered on a pad of celite, rinsed with DCM, then rinsed with EtOH. The volatiles were removed in vacuo to give 7-((4-phenoxybenzoyl)glycyl)-7-azabicyclo[2.2.1]heptane-1-carboxylic acid (0.059 g, 0.15 mmol, Yield 99.289%) as a colorless solid, which was used as is in the next step. HPLC-MS (ESI): m/z [M+H]⁺: 417. Rt=3.09 min.

To a solution of 7-((4-phenoxybenzoyl)glycyl)-7-azabicyclo[2.2.1]heptane-1-carboxylic acid (51.7 mg, 0.131 mmol, 1 equiv.) in DMF (1.5 mL) cooled in an ice/NaCl bath was added EDCI hydrochloride (31.085 mg, 0.157 mmol, 1.2 equiv.) followed by HOAT (18.021 mg, 0.131 mmol, 1 equiv.). The reaction was stirred for 30 mins. 5-(aminomethyl)thiophene-3-carboximidamide dihydrochloride (E; 37.51 mg, 0.131 mmol, 1 equiv.) was added followed by dropwise addition of 4-methyl morpholine (39.775 mg, 0.043 mL, 0.393 mmol, 3 equiv.). The reaction was stirred for 20 h and allowed to warm to RT. The reaction was then concentrated and partitioned between DCM and NaHCO₃ (sat). The organic layer was separated, and the aqueous layer extracted 3× with DCM. The combined organic layers were washed with brine, dried over Na₂SO₄ filtered and concentrated in vacuo. DCM was added to produce a slurry which was filtered. The solids (48 mg) were collected by filtration. The filtrate was suspended on silica gel and purified on a Redisep gold column (12 g) using a gradient of 15 to 70%; A=ethyl acetate/B=20% MeOH In EA with 15% B at t=0. Chromatography provided a further 12 mg for a combined yield of N-((4-(N-acetoxycarbamimidoyl)thiophen-2-yl)methyl)-7-((4-phenoxybenzoyl)glycyl)-7-azabicyclo[2.2.1]heptane-1-carboxamide (F; 0.06 g, 0.102 mmol, Yield 77.9%). HPLC-MS (ESI): m/z [M+23]⁺: 612.2.

Step 4. N-((4-carbamimidoylthiophen-2-yl)methyl)-7-((4-phenoxybenzoyl)glycyl)-7-azabicyclo[2.2.1]heptane-1-carboxamide (Compound 16). A flask containing N-((4-(N-acetoxycarbamimidoyl)thiophen-2-yl)methyl)-7-((4-phenoxybenzoyl)glycyl)-7-azabicyclo[2.2.1]heptane-1-carboxamide (F; 12 mg, 0.02 mmol, 1 equiv.) was evacuated and placed under argon followed by the addition of acetic acid (1 mL). Argon was bubbled into the solution for 10 mins. Pd/C 10% (2.166 mg, 0.002 mmol, 0.1 equiv.) was then added and argon was bubbled into the solution for 5 mins. H₂ was bubbled into the reaction for 5 mins, after which the reaction was allowed to react under a H₂ atmosphere for 20 h. The reaction mixture was concentrated, MeOH was added, and the resulting mixture was filtered through a 45 um Teflon syringe filter and concentrated once again. MeOH (1 ml) was added and the solution cooled to 0° C. 4 N HCl in dioxane (25.438 μL, 0.102 mmol, 5 equiv.) was added and the reaction was stirred for 2 h, after which it was concentrated. The reaction was then suspended in EtOAc and stirred for 16 h. The suspension was collected by filtration providing 7 mg of crude product, which was further purified by preparative TLC using 17/83 MeOH/DCM to provide N-((4-carbamimidoylthiophen-2-yl)methyl)-7-((4-phenoxybenzoyl)glycyl)-7-azabicyclo[2.2.1]heptane-1-carboxamide hydrochloride (Compound 16; 3.2 mg, 0.0056 mmol, 27%) as a beige solid. Purity 94.3%, Rt=7.86 min). ¹H NMR (CH₃OD, 400 MHz): 1H NMR (400 MHz, CD₃OD): δ 8.18 (1H, s), 7.84 (2H, d, J=8.5 Hz), 7.47 (1H, s), 7.42 (2H, t, J=7.8 Hz), 7.20 (1H, t, J=7.4 Hz), 7.01 (2H, d, J=8.5 Hz), 4.59 (3H, s), 4.19 (2H, s), 1.98-2.12 (4H, m), 1.80-1.85 (2H, m), 1.67-1.72 (2H, m). HPLC-MS (ESI): m/z [M+H]⁺: 532.2, Rt=2.39 min.

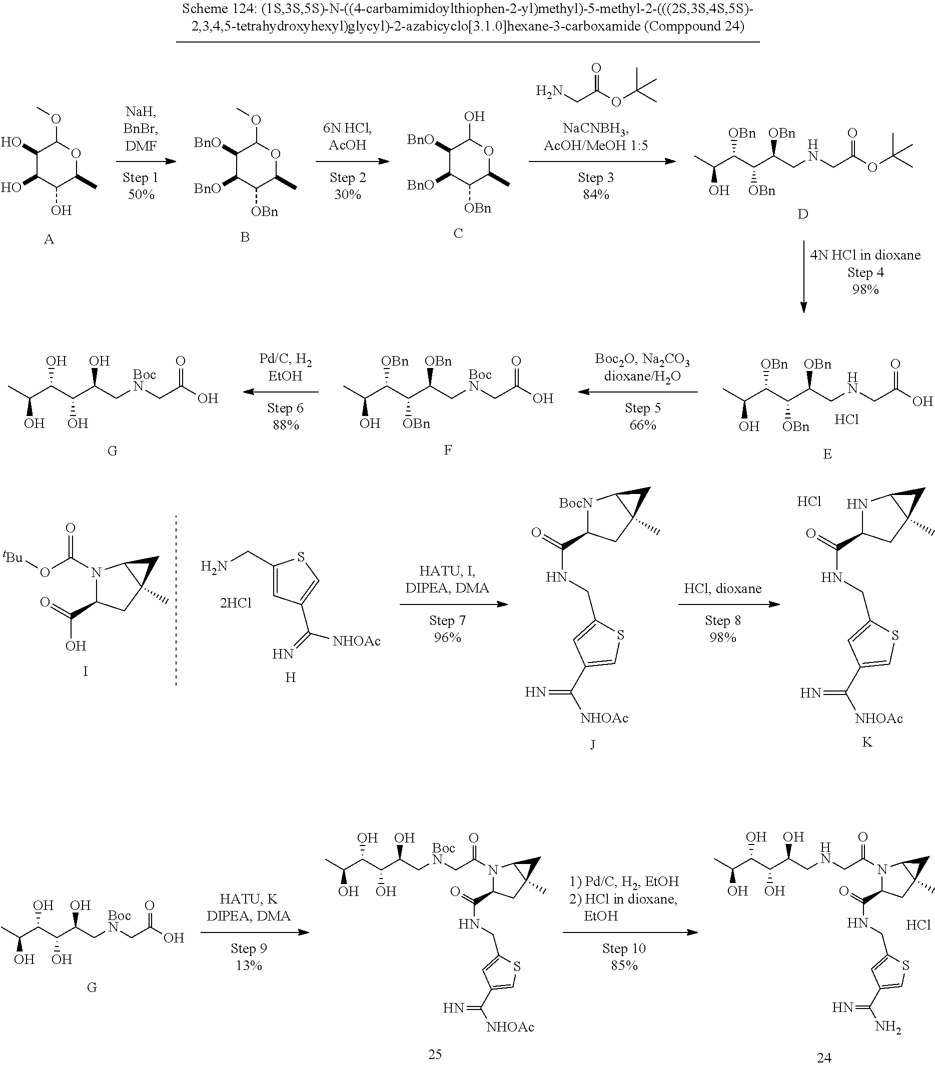

Step 1: (3R,4R,5S,6S)-3,4,5-tris(benzyloxy)-2-methoxy-6-methyltetrahydro-2H-pyran (B). To a solution (3R,4R,5R,6S)-2-methoxy-6-methyltetrahydro-2H-pyran-3,4,5-triol (A; 3 g, 16.8 mmol, 1 equiv.) in DMF (168 mL, 0.1 M, 56.1 Vols) at 0° C. was added 60% sodium hydride in mineral oil (0.673 g, 52.19 mmol, 3.1 equiv.). The reaction mixture was stirred for 60 minutes. Benzyl bromide (6 mL, 50.5 mmol, 3 equiv.) was added dropwise. The reaction mixture was allowed to warm to room temperature and stirred for 24 h. The excess sodium hydride was quenched with MeOH. The residue was dissolved in ethyl acetate and water. The organic layer was separated, washed with water then with a saturated NaCl solution, dried over Na₂SO₄ and concentrated in vacuo. The crude mixture was purified by normal phase chromatography. ISCO, (80 g). Solvents: EtOAc/Hexanes. Gradient: 0% EtOAc (2CV), 0% to 10% EtOAc (1CV), 10% EtOAc (8 CV) affording (3R,4R,5S,6S)-3,4,5-tris(benzyloxy)-2-methoxy-6-methyltetrahydro-2H-pyran (B; 3.8 g, 8.472 mmol, Yield 50.3%). HPLC-MS (ESI) (m/z) [M+Na]⁺: 471.1, RT=4.516 min.

Step 2: (3R,4R,5S,6S)-3,4,5-tris(benzyloxy)-6-methyltetrahydro-2H-pyran-2-ol (C). A solution of (3R,4R,5S,6S)-3,4,5-tris(benzyloxy)-2-methoxy-6-methyltetrahydro-2H-pyran (B; 0.82 g, 1.828 mmol, 1 equiv.) in glacial acetic acid (5.5 mL) was stirred 30 min at RT before the addition of 4 N HCl in dioxane (0.5 mL). The resulting mixture was stirred at 80-85° C. for 30 min. The reaction mixture was poured into 100 mL of ice and water, and the mixture was extracted with CH₂Cl₂ (3×100 ml). The combined extracts were washed successively with saturated aqueous NaHCO₃ and saturated brine, dried over anhydrous Na₂SO₄, and concentrated in vacuo. The residue was purified by flash chromatography, ISCO (40 g). Solvents: EtOAc/hexanes, gradient: 50% EtOAc (15 min), 70% EtOAc (9 min) to afford (3R,4R,5S,6S)-3,4,5-tris(benzyloxy)-6-methyltetrahydro-2H-pyran-2-ol (3; 0.24 g, 0.552 mmol, Yield 30.2%) as a colorless oil. HPLC-MS (ESI) (m/z) [M+Na]⁺: 475.2, Rt=4.117 min.

Step 3: tert-butyl ((2S,3S,4S,5S)-2,3,4-tris(benzyloxy)-5-hydroxyhexyl)glycinate (D). To a solution of (3R,4R,5S,6S)-3,4,5-tris(benzyloxy)-6-methyltetrahydro-2H-pyran-2-ol (C; 720 mg, 1.657 mmol, 1 equiv.) and tert-butyl glycinate (434.702 mg, 3.314 mmol, 2 equiv.) in anhydrous methanol (16.5 mL), was added glacial acetic acid (5.5 mL) followed by molecular sieves (3 Å, 200 mg). The mixture was stirred 10 min at room temperature. NaBH₃CN (151.189 mg, 2.485 mmol, 1.5 equiv.) was added in one portion and the mixture stirred 3 d at RT. The reaction mixture was concentrated in vacuo to a minimum volume. The residue was dissolved in CH₂Cl₂ and neutralized with 1 N NaOH. The organic phase was dried over Na₂SO₄ and concentrated. The residue was purified by flash chromatography: ISCO, (40 g), solvents: EtOAc/Hexanes, gradient: 40% EtOAc (9 min), then 50% EtOAc (15 min), affording tert-butyl ((2S,3S,4S,5S)-2,3,4-tris(benzyloxy)-5-hydroxyhexyl)glycinate (D; 770 mg, 1.401 mmol, Yield 84.5%) as a transparent syrup. HPLC-MS (ESI) (m/z) [M+H]⁺: 550.3, Rt=3.054 min.

Step 4: ((2S,3S,4S,5S)-2,3,4-tris(benzyloxy)-5-hydroxyhexyl)glycine hydrochloride (E). A solution of tert-butyl ((2S,3S,4S,5S)-2,3,4-tris(benzyloxy)-5-hydroxyhexyl)glycinate (D; 770 mg, 1.4 mmol, 1 equiv.) in 4 N HCl in dioxane (4.6 mL, 0.3 M, 6.0 Vols) was stirred overnight at room temperature. The solvent was removed in vacuo affording ((2S,3S,4S,5S)-2,3,4-tris(benzyloxy)-5-hydroxyhexyl)glycine hydrochloride (E; 730 mg, 1.377 mmol, Yield 98.3%) as a white foam. HPLC-MS (ESI) (m/z) [M+H]⁺: 494.2, Rt=3.116 min.

Step 5: N-(tert-butoxycarbonyl)-N-((2S,3S,4S,5S)-2,3,4-tris(benzyloxy)-5-hydroxyhexyl)-glycine (F). To a 25 mL round bottom flask was added ((2S,3S,4S,5S)-2,3,4-tris(benzyloxy)-5-hydroxyhexyl)glycine hydrochloride (D; 148 mg, 0.279 mmol, 1 equiv.), which was dissolved in dioxane (1 mL, 0.279 M, 6.75 Vols). The solution was placed inside an ice bath at 0° C. and stirred magnetically. To this mixture was added di-tert-butyl dicarbonate (79.22 mg, 0.363 mmol, 1.3 equiv.) followed by an aqueous solution of sodium carbonate (118.375 mg, 1.117 mmol, 4 equiv.) in 1 mL water. The mixture was warmed to room temperature and stirred for 6 hours. Upon completion, the volatiles were removed under reduced pressure and the remaining material was diluted in ethyl acetate (15 mL). The organic phase was washed with water, then brine, and dried over sodium sulfate. The extract was concentrated under vacuum and purified by flash chromatography using hexanes/EtOAc/1% formic acid (from 0% to 100% EtOAc). The fractions containing product were combined and concentrated under vacuum to afford N-(tert-butoxycarbonyl)-N-((2S,3S,4S,5S)-2,3,4-tris(benzyloxy)-5-hydroxyhexyl)glycine (F; 110 mg, 0.185 mmol, Yield 66.3%) as a white solid. HPLC-MS (ESI) (m/z) [M+Na]⁺: 616.3.

Step 6: N-(tert-butoxycarbonyl)-N-((2S,3S,4S,5S)-2,3,4,5-tetrahydroxyhexyl)glycine (G). To a 25 mL round bottom flask was added N-(tert-butoxycarbonyl)-N-((2S,3S,4S,5S)-2,3,4-tris(benzyloxy)-5-hydroxyhexyl)glycine (F; 135 mg, 0.227 mmol, 1 equiv.), which was dissolved in ethanol (1.5 mL, 0.152 M, 11.1 Vols). The flask was equipped with a rubber septum and the overhead was purged with nitrogen gas. Pd/C 10% w (60.495 mg, 0.057 mmol, 0.25 equiv.) was then added to the mixture. The headspace was then purged with H₂ gas for 2 minutes. A hydrogen balloon was then attached to the system. The mixture was stirred at room temperature for 18 hours. Finally, the mixture was diluted with 3 mL ethanol and passed through a syringe filter. The residue was concentrated in vacuo to afford N-(tert-butoxycarbonyl)-N-((2S,3S,4S,5S)-2,3,4,5-tetrahydroxyhexyl)glycine (G; 65 mg, 0.201 mmol, Yield 88%) as a white solid. HPLC-MS (ESI) (m/z) [M+Na]⁺: 346.1.

Step 7: tert-butyl (1S,3S,5S)-3-(((4-(N-acetoxycarbamimidoyl)thiophen-2-yl)methyl)-carbamoyl)-5-methyl-2-azabicyclo[3.1.0]hexane-2-carboxylate (J). To a 25 mL round bottom flask was added (1 S,3S,5S)-2-(tert-butoxycarbonyl)-5-methyl-2-azabicyclo[3.1.0]hexane-3-carboxylic acid (I; 200 mg, 0.829 mmol, 1 equiv.) and 1-[bis(dimethylamino)methylene]-1H-1,2,3-triazolo[4,5-b]pyridinium 3-oxide hexafluorophosphate (315.168 mg, 0.829 mmol, 1 equiv.). The mixture was dissolved in N,N-dimethylacetamide (4 mL, 0.207 M, 20 Vols) and stirred magnetically at 0° C. in an ice bath for 20 minutes. To this solution was added N-acetoxy-5-(aminomethyl)thiophene-3-carboximidamide dihydrochloride (284.644 mg, 0.995 mmol, 1.2 equiv.) followed by dropwise addition of N-ethyldiisopropylamine (535.6 mg, 0.724 mL, 4.14 mmol, 5 equiv.). The mixture was stirred at 0° C. for 2 hours. Upon completion, the crude mixture was diluted with EtOAc (25 mL) and the organic phase was washed with water and then brine. The organic layer was dried over sodium sulfate and the solvent was evaporated under reduced pressure. The residue was purified by flash chromatography on silica gel using a gradient of hexanes/EtOAc (0% to 100%). Fractions containing product were combined and concentrated in vacuo, affording tert-butyl (1 S,3S,5S)-3-(((4-(N-acetoxycarbamimidoyl)thiophen-2-yl)methyl)carbamoyl)-5-methyl-2-azabicyclo[3.1.0]hexane-2-carboxylate (J; 348.6 mg, 0.799 mmol, Yield 96.342%) as a white solid. HPLC-MS (ESI) (m/z) [M+Na]⁺: 459.1.

Step 8: (1S,3S,5S)-N-((4-(N-acetoxycarbamimidoyl)thiophen-2-yl)methyl)-5-methyl-2-azabicyclo[3.1.0]hexane-3-carboxamide hydrochloride (K). A 25 mL RBF was charged with tert-butyl (1 S,3S,5S)-3-[({4-[N-(acetyloxy)carbamimidoyl]thiophen-2-yl}methyl)carbamoyl]-5-methyl-2-azabicyclo[3.1.0]hexane-2-carboxylate (175 mg, 0.401 mmol, 1 equiv.). The solid was partially dissolved in dioxane (0.5 mL) and placed inside an ice bath. To this solution was added HCl 4 M in dioxane (219.246 mg, 1.503 mL, 6.013 mmol, 15 equiv.). The mixture was stirred at 0° C. for 2 hours, after which a viscous solid was formed. The volatiles were then removed under reduced pressure. The procedure was repeated one more time to ensure full deprotection. Upon completion, the mixture was concentrated in vacuo to afford (1S,3S,5S)-N-((4-(N-acetoxycarbamimidoyl)thiophen-2-yl)methyl)-5-methyl-2-azabicyclo[3.1.0]hexane-3-carboxamide hydrochloride (145 mg, 0.389 mmol, Yield 97.561%) as a white powder. HPLC-MS (ESI) (m/z) [M+H]⁺: 337.1.

Step 9: tert-butyl (2-((1S,3S,5S)-3-(((4-(N-acetoxycarbamimidoyl)thiophen-2-yl)methyl)-carbamoyl)-5-methyl-2-azabicyclo[3.1.0]hexan-2-yl)-2-oxoethyl)((2S,3S,4S,5S)-2,3,4,5-tetrahydroxyhexyl)carbamate (Compound 25). A 25 mL RBF was charged with N-(tert-butoxycarbonyl)-N-((2S,3S,4S,5S)-2,3,4,5-tetrahydroxyhexyl)glycine (G; 107 mg, 0.331 mmol, 1 equiv.) and 1-[bis(dimethylamino)methylene]-1H-1,2,3-triazolo[4,5-b]pyridinium 3-oxide hexafluorophosphate (125.825 mg, 0.331 mmol, 1 equiv.). The mixture was dissolved in N,N-dimethyl-acetamide (DMAc) (2.5 mL, 0.132 M, 23.364 Vols) and placed inside an ice bath at 0° C. The solution was stirred for 5 minutes and (1 S,3S,5S)-N-((4-(N-acetoxycarbamimidoyl)thiophen-2-yl)methyl)-5-methyl-2-azabicyclo[3.1.0]-hexane-3-carboxamide hydrochloride (K; 155.854 mg, 0.463 mmol, 1.4 equiv.) was added. The mixture was stirred for another 5 minutes and then N-ethyldiisopropylamine (213.84 mg, 0.289 mL, 1.655 mmol, 5 equiv.) was added dropwise. The reaction was stirred at 0° C. for 2 hours. Upon completion, the resulting crude material was concentrated under vacuum and the residue was purified by SFC-HPLC. tert-butyl (2-((1 S,3S,5S)-3-(((4-(N-acetoxycarbamimidoyl)thiophen-2-yl)methyl)carbamoyl)-5-methyl-2-azabicyclo-[3.1.0]hexan-2-yl)-2-oxoethyl)((2S,3S,4S,5S)-2,3,4,5-tetrahydroxyhexyl)carbamate (Compound 25; 28 mg, 0.044 mmol, 13.185% Yield) was obtained as a pale-yellow solid. HPLC-MS (ESI) (m/z) [M+H]⁺: 642.4.

Step 10: (1S,3S,5S)-N-((4-carbamimidoylthiophen-2-yl)methyl)-5-methyl-2-(((2S,3S,4S,5S)-2,3,4,5-tetrahydroxyhexyl)glycyl)-2-azabicyclo[3.1.0]hexane-3-carboxamide hydrochloride (Compound 24). To a 25 mL round bottom flask was added tert-butyl (2-((1 S,3S,5S)-3-(((4-(N-acetoxycarbamimidoyl)thiophen-2-yl)methyl)carbamoyl)-5-methyl-2-azabicyclo[3.1.0]hexan-2-yl)-2-oxoethyl)((2S,3S,4S,5S)-2,3,4,5-tetrahydroxyhexyl)carbamate (20 mg, 0.031 mmol, 1 equiv.) and dissolved in ethanol (1 mL, 0.031 M, 50 Vols). The flask was equipped with a rubber septum and the overhead was purged with nitrogen gas. Pd on carbon (6.633 mg, 0.006 mmol, 0.2 equiv.) was then added to the mixture. The headspace was then purged with hydrogen gas for 2 minutes. A hydrogen balloon was then attached to the system. The mixture was stirred at room temperature for 2 hours. Finally, the mixture was diluted with 3 mL ethanol and passed through a syringe filter. The residue was concentrated in vacuo to afford a colorless oil, which was used without further purification.

To a 25 mL round bottom flask containing the product from the previous reaction (18 mg, 0.031 mmol, 1 equiv.) was added ethanol (0.2 mL, 0.154 M, 11.1 Vols). The solution was placed inside an ice bath and cooled to 0° C. To this solution was added HCl (0.022 g, 0.154 mL, 0.617 mmol, 20 equiv.; 4 M in dioxane). The solution was stirred at 0° C. and allowed to warm to room temperature over 6 hours. The reaction was concentrated in vacuo and the resulting oil was triturated with MTBE (1 mL). The volatiles were removed under vacuum to afford (1 S,3S,5S)-N-[(4-carbamimidoylthiophen-2-yl)methyl]-5-methyl-2-(2-{[(2S,3S,4S,5S)-2,3,4,5-tetrahydroxyhexyl]amino}acetyl)-2-azabicyclo-[3.1.0]hexane-3-carboxamide hydrochloride (Compound 24; 13.6 mg, 0.026 mmol, Yield 85%) as pale-yellow crystals. HPLC-MS (ESI) (m/z) [M+H]⁺: 484.2.

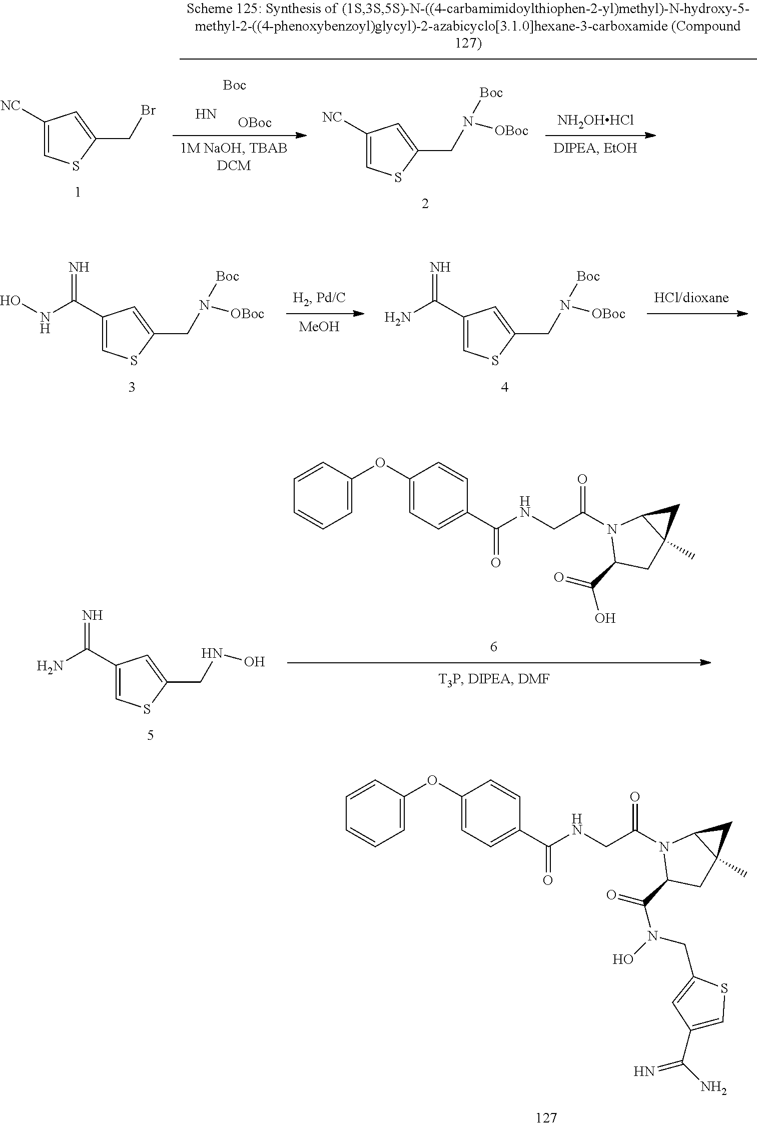

### Step 1: tert-butyl ((tert-butoxycarbonyl)oxy)((4-cyanothiophen-2-yl)methyl)carbamate (2)

To a mixture of 5-(bromomethyl)thiophene-3-carbonitrile (300 mg, 1.49 mmol) and tert-butyl ((tert-butoxycarbonyl)oxy)carbamate (384 mg, 1.64 mmol) CH₂Cl₂ (30 mL) was added 1 M aq. NaOH solution (13.5 mL) and TBAB (30 mg, 0.09 mmol) and the mixture was stirred at 25° C. for 16 hours. The mixture was diluted with CH₂Cl₂, washed with water and brine, dried over anhydrous Na₂SO₄, filtered and concentrated to dryness under reduced pressure. The residue was purified by flash chromatography on silica gel (petroleum ether:ethyl acetate=94:6) to give tert-butyl ((tert-butoxycarbonyl)oxy)((4-cyanothiophen-2-yl)methyl)carbamate (460 mg, yield 87.1%) as colorless oil. LC/MS (ESI) m/z: 355 (M+H)⁺.

### Step 2: tert-butyl ((tert-butoxycarbonyl)oxy)((4-(N-hydroxycarbamimidoyl)thiophen-2-yl)methyl)carbamate (3)

To a mixture of tert-butyl ((tert-butoxycarbonyl)oxy)((4-cyanothiophen-2-yl)methyl)carbamate (460 mg, 1.30 mmol) and NH₂OH·HCl (226 mg, 3.25 mmol) in EtOH (10 mL) was added DIPEA (0.65 mL, 3.90 mmol) and the mixture was stirred at 25° C. for 16 hours. The mixture was concentrated to dryness under reduced pressure and the residue was purified by column chromatography on silica gel (DCM:MeOH=96:4) to give tert-butyl ((tert-butoxycarbonyl)oxy) ((4-(N-hydroxycarbamimidoyl)thiophen-2-yl)methyl) carbamate (400 mg, yield 79.7%) as colorless oil. LC/MS (ESI) m/z: 388 (M+H)⁺.

### Step 3: tert-butyl ((tert-butoxycarbonyl)oxy)((4-carbamimidoylthiophen-2-yl)methyl)carbamate (4)

To a solution of tert-butyl ((tert-butoxycarbonyl)oxy)((4-(N-hydroxycarbamimidoyl)thiophen-2-yl)methyl)carbamate (400 mg, 1.03 mmol) in MeOH (5 mL) was added Pd/C (20 mg, 10% wt) and the mixture was degassed 3 times under N₂ atmosphere and stirred under a H₂ balloon at room temperature for 2 hours. The mixture was filtered and the filtrate was concentrated to dryness under reduced pressure to give compound 4 (360 mg, yield 94.5%) as colorless oil. The crude product was used directly in the next step without further purification. LC/MS (ESI) (m/z): 372 (M+H)⁺.

### Step 4: 5-((hydroxyamino)methyl)thiophene-3-carboximidamide (5)

To a solution of tert-butyl ((tert-butoxycarbonyl)oxy)((4-carbamimidoylthiophen-2-yl)methyl)carbamate (360 mg, 0.97 mmol) in HCl/1,4-dioxane (5 mL) at 25° C. and the mixture was stirred at 25° C. for 2 hours. The reaction mixture was concentrated to dryness under reduced pressure to give 5-((hydroxyamino)methyl) thiophene-3-carboximidamide (150 mg, yield 90.4%) as white solid, which was used directly in the next step without further purification. LC/MS (ESI) m/z: 172 (M+H)⁺.

### Step 5: (1S,3S,5S)-N-((4-carbamimidoylthiophen-2-yl)methyl)-N-hydroxy-5-methyl-2-((4-phenoxybenzoyl)glycyl)-2-azabicyclo[3.1.0]hexane-3-carboxamide (Compound 127)

To a mixture of 5-((hydroxyamino)methyl) thiophene-3-carboximidamide (130 mg, 0.75 mmol) and (1S,3S,5S)-5-methyl-2-((4-phenoxybenzoyl)glycyl)-2-azabicyclo[3.1.0]hexane-3-carboxylic acid (100 mg, 0.25 mmol) in DMF (5 mL) was added DIPEA (0.25 mL, 1.50 mmol) and T₃P (484 mg, 0.75 mmol, 50% wt in ethyl acetate) at 0° C. under N₂ atmosphere and the mixture was stirred at 25° C. for 3 hours. The mixture was diluted with water and extracted with CHCl₃/i-PrOH (5×10 mL, V/V=3/1). The combined organic layers were dried over anhydrous Na₂SO₄, filtered, and concentrated to dryness under reduced pressure. The residue was purified by prep-HPLC to give Compound 127 (1S,3S,5S)-N-((4-carbamimidoylthiophen-2-yl)methyl)-N-hydroxy-5-methyl-2-((4-phenoxybenzoyl)glycyl)-2-azabicyclo[3.1.0]hexane-3-carboxamide (10 mg, yield 7.2%) as white solid. ¹H NMR (400 MHz, CD₃OD) δ 8.55 (s, 2H), 8.30 (dd, J=11.8, 1.6 Hz, 1H), 7.90-7.84 (m, 2H), 7.53 (d, J=18.3 Hz, 1H), 7.45-7.39 (m, 2H), 7.21 (dd, J=10.6, 4.2 Hz, 1H), 7.06 (dd, J=8.6, 1.0 Hz, 2H), 7.04-6.98 (m, 2H), 5.36 (dd, J=11.6, 3.5 Hz, 1H), 4.91 (d, J=5.5 Hz, 2H), 4.56 (d, J=16.8 Hz, 1H), 4.23 (d, J=16.7 Hz, 1H), 3.41 (dd, J=5.9, 2.4 Hz, 1H), 2.48 (t, J=13.3 Hz, 1H), 2.01 (dd, J=13.6, 3.6 Hz, 1H), 1.29 (s, 3H), 1.25 (d, J=4.4 Hz, 1H), 0.77 (t, J=5.2 Hz, 1H). LC/MS (ESI) m/z: 548 (M+H)⁺.

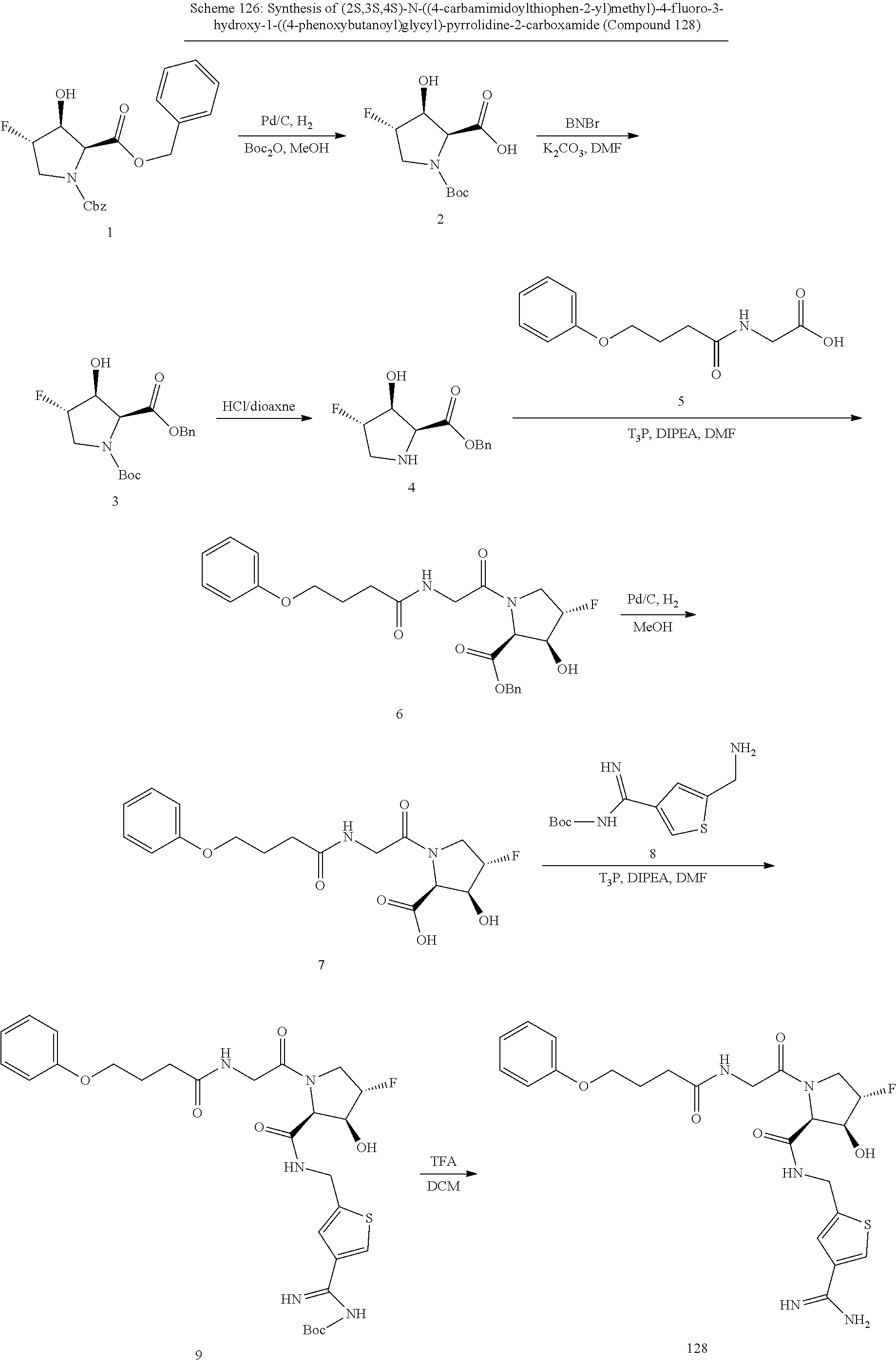

### Step 1: (2S,3S,4S)-1-(tert-butoxycarbonyl)-4-fluoro-3-hydroxypyrrolidine-2-carboxylic acid (2)

To a solution of dibenzyl (2S,3S,4S)-4-fluoro-3-hydroxypyrrolidine-1,2-dicarboxylate (200 mg, 0.54 mmol) in MeOH (5 mL) was added Pd/C (40 mg, 10% wt) and Boc₂₀ (175 mg, 0.81 mmol). The mixture was degassed under N₂ atmosphere for three times and stirred under a H₂ balloon at room temperature for 1.5 hours. The mixture was filtered and the filtrate was concentrated to dryness under reduced pressure to give (2S,3S,4S)-1-(tert-butoxycarbonyl)-4-fluoro-3-hydroxypyrrolidine-2-carboxylic acid (130 mg, yield 97.7%) as colorless oil. The crude product was used directly in next step. LC/MS (ESI) (m/z): 250 (M+H)⁺.

### Step 2: 2-benzyl 1-(tert-butyl) (2S,3S,4S)-4-fluoro-3-hydroxypyrrolidine-1,2-dicarboxylate (3)

To a solution of (2S,3S,4S)-1-(tert-butoxycarbonyl)-4-fluoro-3-hydroxypyrrolidine-2-carboxylic acid (130.0 mg, 0.52 mmol) in DMF (5.0 mL) was added BnBr (0.09 mL, 0.78 mmol) and K₂CO₃ (216.0 mg, 1.56 mol) at 0° C. under N₂ atmosphere and the mixture was stirred at 25° C. for 16 hours. The mixture was diluted with EtOAc, extracted with EtOAc twice and washed with saturated aq.NH₄Cl solution. The combined organic layers were dried over anhydrous Na₂SO₄, filtered and concentrated to dryness under reduced pressure. The residue was purified by column chromatography on silica gel (PE:EtOAc=1:1) to give 2-benzyl 1-(tert-butyl) (2S,3S,4S)-4-fluoro-3-hydroxypyrrolidine-1,2-dicarboxylate (160 mg, yield 90.4%) as colorless oil. LC/MS (ESI) m/z: 340 (M+H)⁺.

### Step 3: benzyl (2S,3S,4S)-4-fluoro-3-hydroxypyrrolidine-2-carboxylate (4)

To a solution of 2-benzyl 1-(tert-butyl) (2S,3S,4S)-4-fluoro-3-hydroxypyrrolidine-1,2-dicarboxylate (160 mg, 0.47 mmol) in HCl/1,4-dioxane (5 mL) at 25° C. and the mixture was stirred at 25° C. for 2 hours. The reaction mixture was concentrated to dryness under reduced pressure to give benzyl (2S,3S,4S)-4-fluoro-3-hydroxypyrrolidine-2-carboxylate (110 mg, yield 98.2%) as brown oil, which was used directly in the next step without further purification. LC/MS (ESI) m/z: 240 (M+H)⁺.

### Step 4: benzyl (2S,3S,4S)-4-fluoro-3-hydroxy-1-((4-phenoxybutanoyl)glycyl)pyrrolidine-2-carboxylate (6)

To a mixture of benzyl (2S,3S,4S)-4-fluoro-3-hydroxypyrrolidine-2-carboxylate (127 mg, 0.53 mmol) and (4-phenoxybutanoyl)glycine (138 mg, 0.58 mmol) in DMF (5 mL) was added DIPEA (0.52 mL, 3.18 mmol) and T₃P (1.0 g, 1.59 mmol) at 0° C. under N₂ atmosphere and the mixture was stirred at 25° C. for 16 hours. The mixture was diluted with water and extracted with EtOAc twice. The combined organic layers were dried over anhydrous Na₂SO₄, filtered and concentrated to dryness under reduced pressure. The residue was purified by column chromatography on silica gel (DCM:MeOH=20:1) to give benzyl (2S,3S,4S)-4-fluoro-3-hydroxy-1-((4-phenoxybutanoyl)glycyl)pyrrolidine-2-carboxylate (220 mg, yield 90.5%) as colorless oil. LC/MS (ESI) m/z: 459 (M+H)⁺.

### Step 5: (2S,3S,4S)-4-fluoro-3-hydroxy-1-((4-phenoxybutanoyl)glycyl)pyrrolidine-2-carboxylic acid (7)

To a solution of benzyl (2S,3S,4S)-4-fluoro-3-hydroxy-1-((4-phenoxybutanoyl)glycyl) pyrrolidine-2-carboxylate (120 mg, 0.26 mmol) in MeOH (5 mL) was added Pd/C (24 mg, 10% wt). The mixture was degassed under N₂ atmosphere for ten times and stirred under a H₂ balloon at room temperature for 2 hours. The mixture was filtered and the filtrate was concentrated to dryness under reduced pressure to give (2S,3S,4S)-4-fluoro-3-hydroxy-1-((4-phenoxybutanoyl)glycyl)pyrrolidine-2-carboxylic acid (95 mg, yield 98.9%) as colorless oil, which was used directly in next step. LC/MS (ESI) (m/z): 369 (M+H)⁺.

### Step 6: tert-butyl ((5-(((2S,3S,4S)-4-fluoro-3-hydroxy-1-((4-phenoxybutanoyl)glycyl) pyrrolidine-2-carboxamido)methyl)thiophen-3-yl)(imino)methyl)carbamate (9)

To a mixture of (2S,3S,4S)-4-fluoro-3-hydroxy-1-((4-phenoxybutanoyl)glycyl)pyrrolidine-2-carboxylic acid (40 mg, 0.11 mmol) and tert-butyl ((5-(aminomethyl)thiophen-3-yl)(imino)methyl)carbamate (55 mg, 0.22 mmol) in DMF (3 mL) was added DIPEA (0.11 mL, 0.66 mmol) and T₃P (207 mg, 0.33 mmol) at 0° C. under N₂ atmosphere and the mixture was stirred at 25° C. for 16 hours. The mixture was diluted with water and extracted with EtOAc twice. The combined organic layers were dried over anhydrous Na₂SO₄, filtered and concentrated to dryness under reduced pressure. The residue was purified by column chromatography on silica gel (DCM:MeOH=10:1) to give tert-butyl ((5-(((2S,3S,4S)-4-fluoro-3-hydroxy-1-((4-phenoxybutanoyl)glycyl)pyrrolidine-2-carboxamido)methyl)thiophen-3-yl)(imino)methyl) carbamate (20 mg, yield 30.3%) as colorless oil. LC/MS (ESI) m/z: 606 (M+H)⁺.

### Step 7: (2S,3S,4S)-N-((4-carbamimidoylthiophen-2-yl)methyl)-4-fluoro-3-hydroxy-1-((4-phenoxybutanoyl)glycyl)pyrrolidine-2-carboxamide (Compound 128)

To a solution of tert-butyl ((5-(((2S,3S,4S)-4-fluoro-3-hydroxy-1-((4-phenoxybutanoyl)glycyl) pyrrolidine-2-carboxamido)methyl)thiophen-3-yl)(imino)methyl)carbamate (20 mg, 0.03 mmol) in DCM (2 mL) was added TFA (1 mL) at 0° C. and the mixture was stirred at 25° C. for 2 hours. The reaction mixture was concentrated to dryness under reduced pressure and the residue was purified by prep-HPLC to give Compound 128 (2.0 mg, yield 11.8%) as white solid. ¹H NMR (400 MHz, CD₃OD) δ 8.21 (dd, J=8.4, 1.6 Hz, 1H), 7.47 (d, J=27.6 Hz, 1H), 7.28-7.20 (m, 2H), 6.90 (dd, J=7.6, 5.3 Hz, 3H), 5.01 (d, J=51.3 Hz, 1H), 4.71-4.61 (m, 1H), 4.56 (dd, J=10.0, 7.0 Hz, 3H), 4.07-3.88 (m, 6H), 2.47 (dd, J=9.5, 4.9 Hz, 2H), 2.07 (dd, J=13.7, 6.5 Hz, 2H). LC/MS (ESI) m/z: 506 (M+H)⁺.

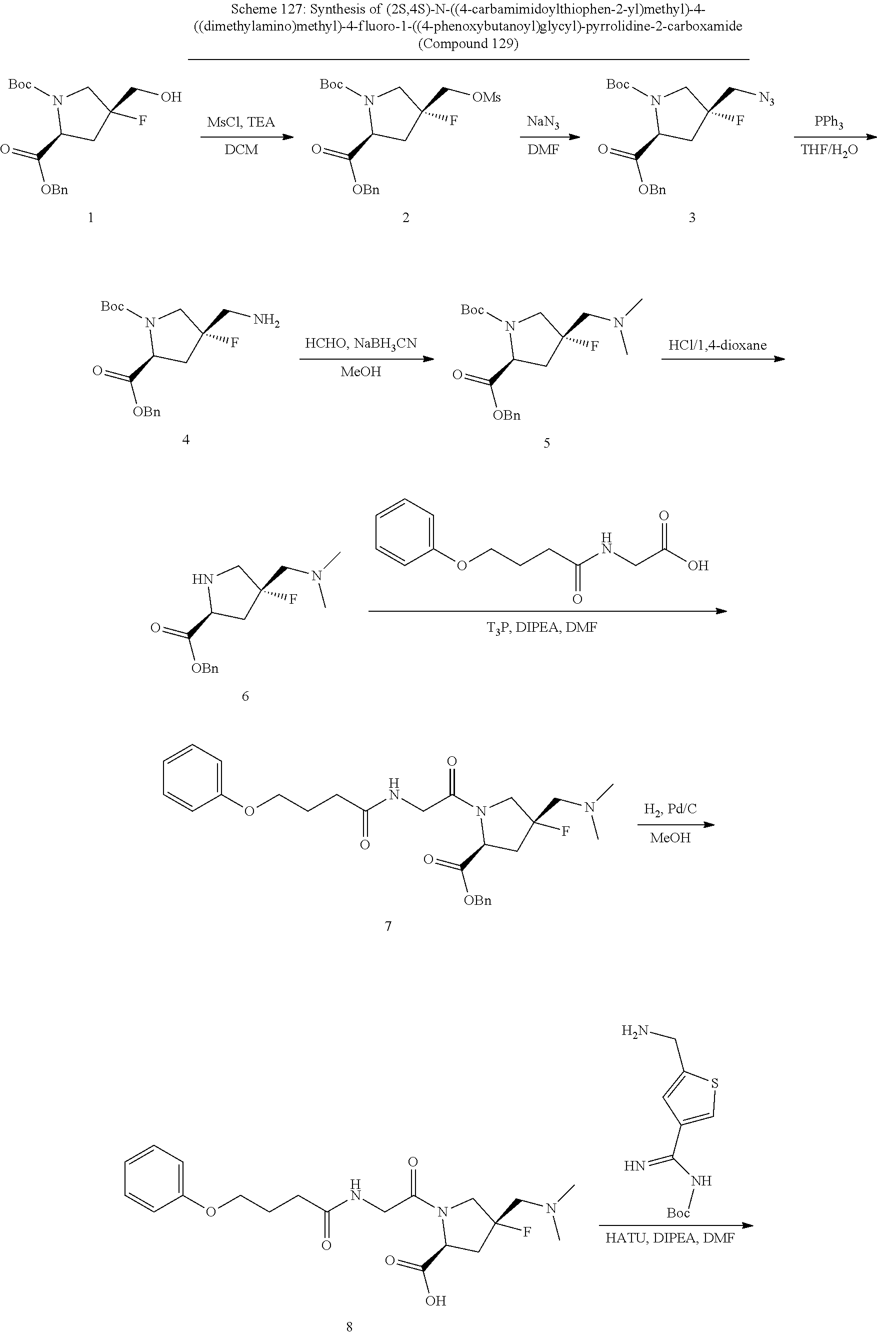

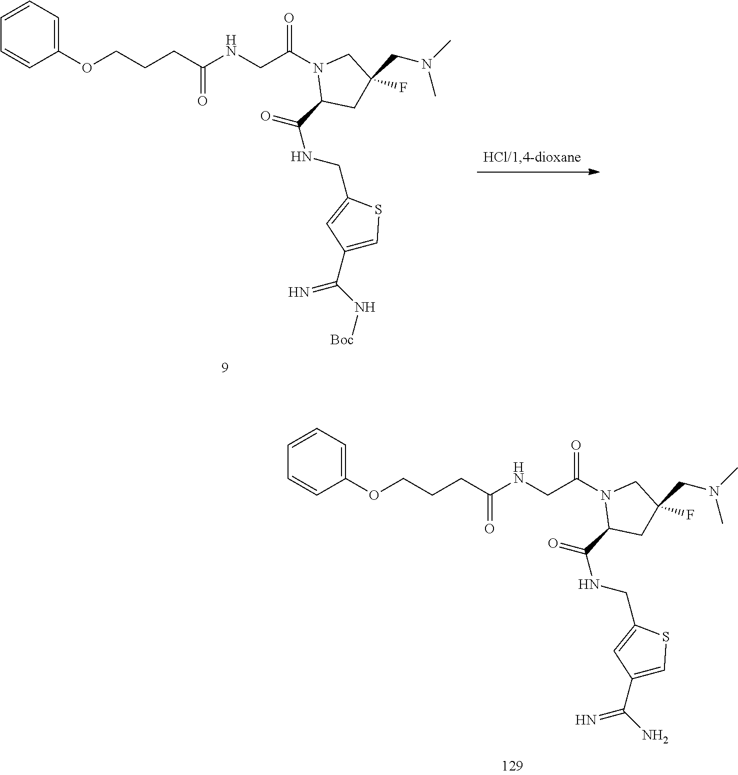 [SMILES: CN(C)C[C@@]1(F)C[C@@H](C(=O)NCc2cc(C(=N)NC(=O)OC(C)(C)C)cs2)N(C(=O)CNC(=O)CCCOc2ccccc2)C1 ; "HCl/1,4-dioxane" ; CN(C)C[C@@]1(F)C[C@@H](C(=O)NCc2cc(C(=N)N)cs2)N(C(=O)CNC(=O)CCCOc2ccccc2)C1]

### Step 1: 2-benzyl 1-(tert-butyl) (2S,4R)-4-fluoro-4-(((methylsulfonyl)oxy)methyl)pyrrolidine-1,2-dicarboxylate (2)

To a solution of 2-benzyl 1-(tert-butyl) (2S,4R)-4-fluoro-4-(hydroxymethyl)pyrrolidine-1,2-dicarboxylate (100 mg, 0.28 mmol) in DCM (3 mL) was added TEA (172 mg, 1.70 mmol) followed by MsCl (97 mg, 0.85 mmol) at 0° C. The reaction mixture was stirred at 0° C. for 2 hours. The mixture was quenched with ice-water and extracted with ethyl acetate twice. The combined organic layers were washed with brine, dried over anhydrous Na₂SO₄, filtered and concentrated to dryness under reduce pressure give 2-benzyl 1-(tert-butyl) (2S,4R)-4-fluoro-4-(((methylsulfonyl)oxy)methyl)pyrrolidine-1,2-dicarboxylate (120 mg, 98.3% yield) as yellow oil, which was used directly in the next step without further purification. LC/MS (ESI) (m/z): 433 (M+H)⁺.

### Step 2: 2-benzyl 1-tert-butyl (2S,4R)-4-(azidomethyl)-4-fluoropyrrolidine-1,2-dicarboxylate (3)

To a solution of 2-benzyl 1-(tert-butyl) (2S,4R)-4-fluoro-4-(((methylsulfonyl)oxy) methyl)pyrrolidine-1,2-dicarboxylate (120 mg, 0.28 mmol) in DMF (3 mL) was added NaN₃ (54 mg, 0.83 mmol) and the mixture was stirred at 65° C. for 5 days. The mixture was diluted with ice-water and extracted with ethyl acetate twice. The combined organic layers were washed with brine, dried over anhydrous Na₂SO₄, filtered and concentrated to dryness under reduced pressure. The residue was purified by flash chromatography on silica gel (petroleum ether:ethyl acetate=30:1 to 15:1) to give 2-benzyl 1-tert-butyl (2S,4R)-4-(azidomethyl)-4-fluoropyrrolidine-1,2-dicarboxylate (60 mg, yield 57.0%) as yellow oil. LC/MS (ESI) m/z: 379 (M+H)⁺.

### Step 3: 2-benzyl 1-tert-butyl (2S,4S)-4-(aminomethyl)-4-fluoropyrrolidine-1,2-dicarboxylate (4)

To a solution of 2-benzyl 1-tert-butyl (2S,4R)-4-(azidomethyl)-4-fluoropyrrolidine-1,2-dicarboxylate (60 mg, 0.16 mmol) in THE (1.6 mL) and H₂O (1.6 mL) was added PPh₃ (83 mg, 0.32 mmol) and the mixture was stirred at 25° C. for 16 hours. The mixture was acidified with 0.5 M aq.HCl solution to pH5 and washed with ethyl acetate twice. The aqueous layer was basified with sat.NaHCO₃ solution to pH8 and extracted with ethyl acetate twice. The combined organic layers were washed with brine, dried over anhydrous Na₂SO₄, filtered and concentrated to dryness under reduced pressure to give 2-benzyl 1-tert-butyl (2S,4S)-4-(aminomethyl)-4-fluoropyrrolidine-1,2-dicarboxylate (55 mg, yield 98.4%) as light yellow oil. LC/MS (ESI) m/z: 353 (M+H)⁺.

### Step 4: 2-benzyl 1-(tert-butyl) (2S,4S)-4-((dimethylamino)methyl)-4-fluoropyrrolidine-1,2-dicarboxylate(5)

To a mixture of 2-benzyl 1-tert-butyl (2S,4S)-4-(aminomethyl)-4-fluoropyrrolidine-1,2-dicarboxylate (55 mg, 0.16 mmol) in MeOH (2 mL) was added NaBH₃CN (20 mg, 0.31 mmol) and formaldehyde (0.04 mL, 0.53 mmol) at 0° C. The reaction was stirred at 25° C. for 2 hours. After completion of the reaction, the reaction mixture was quenched with ice water and extracted with DCM twice. The combined organic layers were washed with brine, dried over anhydrous Na₂SO₄ and concentrated to dryness under reduced pressure. The residue was purified by flash chromatography on silica gel (DCM:MeOH=60:1) to give 2-benzyl 1-(tert-butyl) (2S,4S)-4-((dimethylamino)methyl)-4-fluoropyrrolidine-1,2-dicarboxylate (56 mg, yield 94.3%) as yellow oil. LC/MS (ESI) m/z: 381 (M+H)⁺.

### Step 5: benzyl (2S,4R)-4-[(dimethylamino)methyl]-4-fluoropyrrolidine-2-carboxylate (6)

A mixture of 2-benzyl 1-(tert-butyl) (2S,4S)-4-((dimethylamino)methyl)-4-fluoropyrrolidine-1,2-dicarboxylate (56 mg, 0.15 mmol) in HCl/1,4-dioxane (2 mL) was stirred at room temperature for 1 hour. The reaction mixture was concentrated to dryness under reduced pressure, co-evaporated with DCM and dried under vacuum to give benzyl (2S,4R)-4-[(dimethylamino)methyl]-4-fluoropyrrolidine-2-carboxylate (40 mg, yield 96.9%) as white solid, which was used directly in the next step without further purification. LC/MS (ESI) m/z: 281 (M+H)⁺.

### Step 6: benzyl (2S,4S)-4-[(dimethylamino)methyl]-4-fluoro-1-[2-(4-phenoxybutanamido) acetyl]pyrrolidine-2-carboxylate (7)

To a mixture of benzyl (2S,4R)-4-[(dimethylamino)methyl]-4-fluoropyrrolidine-2-carboxylate (40 mg, 0.14 mmol) and (4-phenoxybutanamido)acetic acid (41 mg, 0.17 mmol) in DMF (3 mL) was added DIPEA (0.15 mL, 0.86 mmol) and T₃P (272 mg, 0.43 mmol, 50% wt in ethyl acetate) at 0° C. under N₂ atmosphere and the mixture was stirred at 25° C. for 2 hours. The mixture was diluted with ethyl acetate and washed with saturated aq.NaHCO₃ solution. The aqueous layer was extracted with ethyl acetate twice. The combined organic layers were washed with brine, dried over anhydrous Na₂SO₄, filtered and concentrated to dryness under reduced pressure. The residue was purified by flash chromatography on silica gel (DCM:MeOH=40:1) to give benzyl (2S,4S)-4-[(dimethylamino)methyl]-4-fluoro-1-[2-(4-phenoxybutanamido) acetyl]pyrrolidine-2-carboxylate (45 mg, yield 63.1%) as yellow oil. LC/MS (ESI) (m/z): 500 (M+H)⁺.

### Step 7: benzyl (2S,4S)-4-[(dimethylamino)methyl]-4-fluoro-1-[2-(4-phenoxybutanamido) acetyl]pyrrolidine-2-carboxylate (8)

To a solution of benzyl (2S,4S)-4-[(dimethylamino)methyl]-4-fluoro-1-[2-(4-phenoxybutanamido) acetyl]pyrrolidine-2-carboxylate (45 mg, 0.09 mmol) in MeOH (3 mL) was added Pd/C (15 mg, 10% wt) at 0° C., and the mixture was degassed under N₂ atmosphere three times and stirred under a H₂ balloon at room temperature for 2 hours. The mixture was filtered and the filtrate was concentrated under reduce pressure to give benzyl (2S,4S)-4-[(dimethylamino)methyl]-4-fluoro-1-[2-(4-phenoxybutanamido)acetyl]pyrrolidine-2-carboxylate (37 mg, 82.2% yield) as light yellow oil, which was used directly in the next step without further purification. LC/MS (ESI) (m/z): 410 (M+H)⁺.

### Step 8: tert-butyl N-[5-({[(2S,4S)-4-[(dimethylamino)methyl]-4-fluoro-1-[2-(4-phenoxybutanamido)acetyl]pyrrolidin-2-yl]formamido}methyl)thiophene-3-carboximidoyl]carbamate (9)

To a mixture of benzyl (2S,4S)-4-[(dimethylamino)methyl]-4-fluoro-1-[2-(4-phenoxybutanamido) acetyl]pyrrolidine-2-carboxylate (35 mg, 0.085 mmol) and tert-butyl N-[5-(aminomethyl)thiophene-3-carboximidoyl]carbamate (33 mg, 0.13 mmol) in DMF (2 mL) was added DIPEA (0.06 mL, 0.86 mmol) and HATU (33 mg, 0.085 mmol) at 0° C. under N₂ atmosphere and the mixture was stirred at 25° C. for 2 hours. The mixture was washed with water and extracted with ethyl acetate twice. The combined organic layers were washed with brine, dried over anhydrous Na₂SO₄, filtered and concentrated to dryness under reduced pressure. The residue was purified by flash chromatography on silica gel (DCM:MeOH=15:1) to give tert-butyl N-[5-({[(2S,4S)-4-[(dimethylamino)methyl]-4-fluoro-1-[2-(4-phenoxybutanamido)acetyl]pyrrolidin-2-yl]formamido}methyl)thiophene-3-carboximidoyl]carbamate (30 mg, yield 54.3%) as white solid. LC/MS (ESI) (m/z): 647 (M+H)⁺.

### Step 9: (2S,4S)-N-[(4-carbamimidoylthiophen-2-yl)methyl]-4-[(dimethylamino)methyl]-4-fluoro-1-[2-(4-phenoxybutanamido)acetyl]pyrrolidine-2-carboxamide (Compound 129)

A solution of tert-butyl N-[5-({[(2S,4S)-4-[(dimethylamino)methyl]-4-fluoro-1-[2-(4-phenoxybutanamido)acetyl]pyrrolidin-2-yl]formamido}methyl)thiophene-3-carboximidoyl]carbamate (30 mg, 0.046 mmol) in HCl/1,4-dioxane (3 mL) was stirred at room temperature for 5 hours. The reaction mixture was concentrated to dryness under reduced pressure. The residue was purified by pre-HPLC to give Compound 129 (1.5 mg, yield 5.9%) as white solid. ¹H NMR (400 MHz, CD₃OD) δ 8.41 (s, 2H), 8.25-8.21 (m, 1H), 7.48 (d, J=18.4 Hz, 1H), 7.24 (t, J=8.0 Hz, 2H), 6.92-6.86 (m, 3H), 4.62-4.51 (m, 3H), 4.12-3.95 (m, 5H), 3.84-3.71 (m, 1H), 2.89-2.81 (m, 2H), 2.64-2.54 (m, 1H), 2.49-2.45 (m, 2H), 2.40 (s, 6H), 2.18-2.02 (m, 3H). LC/MS (ESI) m/z: 547 (M+H)⁺.

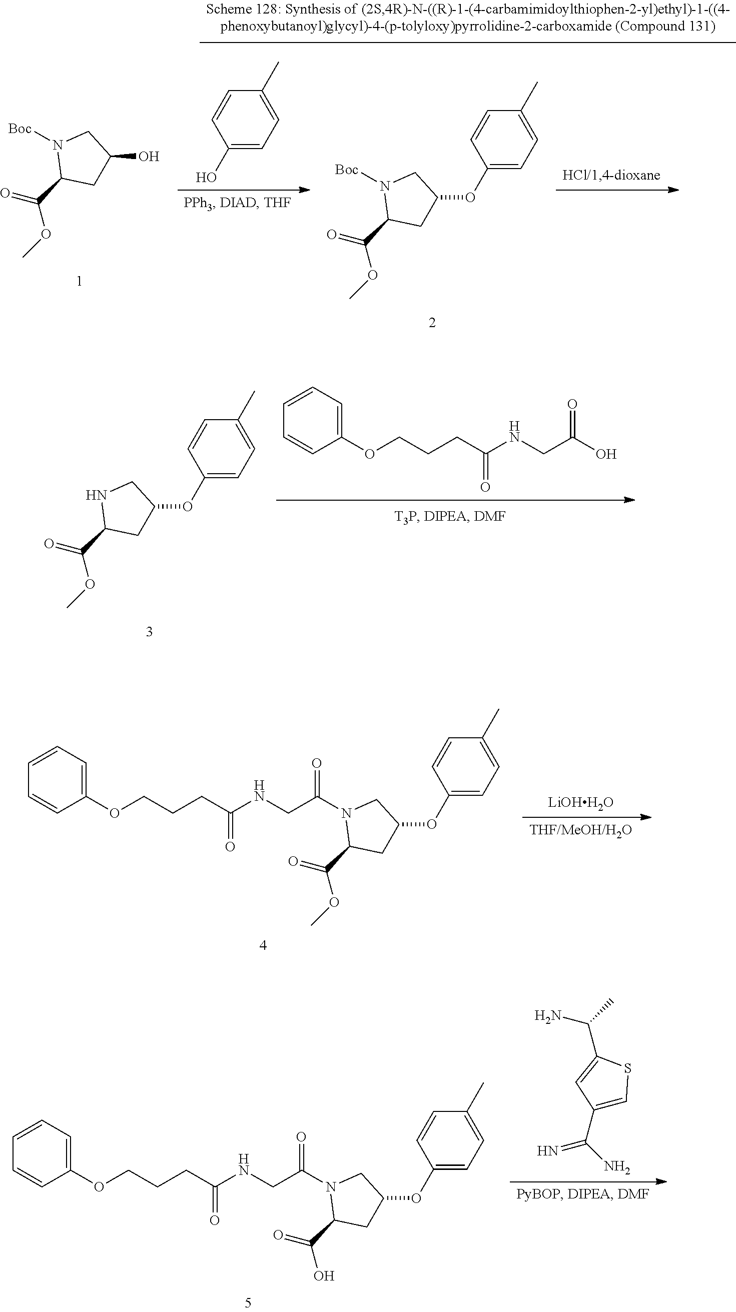

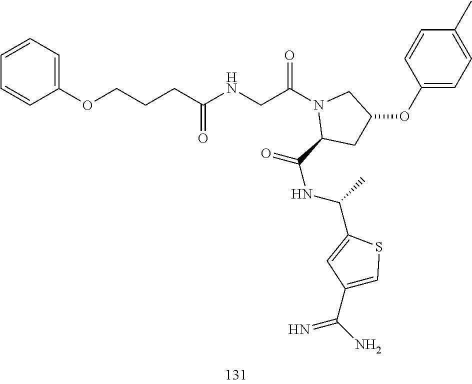 [SMILES: Cc1ccc(O[C@@H]2C[C@@H](C(=O)N[C@H](C)c3cc(C(=N)N)cs3)N(C(=O)CNC(=O)CCCOc3ccccc3)C2)cc1]

### Step 1: 1-(tert-butyl) 2-methyl (2S,4R)-4-(p-tolyloxy)pyrrolidine-1,2-dicarboxylate (2)

To a solution of 1-(tert-butyl) 2-methyl (2S,4S)-4-hydroxypyrrolidine-1,2-dicarboxylate (1 g, 4.08 mmol) in THE (20 mL) was added PPh₃ (1.6 g, 6.12 mmol) at 0° C. p-cresol (661 mg, 6.12 mmol) was added followed by the addition of DIAD (1.2 g, 6.12 mmol) under N₂ atmosphere and the resulting mixture was stirred at room temperature overnight. The mixture was diluted with ethyl acetate and washed with water. The aqueous layer was extracted with ethyl acetate twice and the combined organic layers were washed with brine, dried over anhydrous Na₂SO₄, filtered and concentrated to dryness under reduced pressure. The residue was purified by flash chromatography on silica gel (petroleum ether:ethyl acetate=10:1) to afford 1-(tert-butyl) 2-methyl (2S,4R)-4-(p-tolyloxy)pyrrolidine-1,2-dicarboxylate (880 mg, 64.4% yield) as white solid. LC/MS (ESI) m/z:336 (M+H)⁺.

### Step 2: methyl (2S,4R)-4-(p-tolyloxy)pyrrolidine-2-carboxylate (3)

To a solution of 1-(tert-butyl) 2-methyl (2S,4R)-4-(p-tolyloxy)pyrrolidine-1,2-dicarboxylate (880 mg, 2.62 mmol) in 1,4-dioxane (3 mL) was added HCl/1,4-dioxane (3 mL, 4M) under N₂ atmosphere and the reaction mixture was stirred at room temperature for 2 hours. The mixture was concentrated to dryness under reduced pressure to give methyl (2S,4R)-4-(p-tolyloxy)pyrrolidine-2-carboxylate (610 mg, yield 98.8%) as white solid. LC/MS (ESI) m/z: 236 (M+H)⁺.

### Step 3: methyl (2S,4R)-1-((4-phenoxybutanoyl)glycyl)-4-(p-tolyloxy)pyrrolidine-2-carboxylate (4)

To a solution of methyl (2S,4R)-4-(p-tolyloxy)pyrrolidine-2-carboxylate (610 mg, 2.59 mmol) and (4-phenoxybutanoyl)glycine (615 mg, 2.59 mmol) in DMF (10 mL) was added DIPEA (2.0 g, 15.56 mmol) and T₃P (5.0 g, 7.78 mmol) under N₂ atmosphere at 0° C. The reaction mixture was stirred at room temperature overnight. The mixture was diluted with ethyl acetate and washed with saturated aq.NaHCO₃ solution. The aqueous layer was extracted with ethyl acetate twice and the combined organic layers were washed with brine, dried over anhydrous Na₂SO₄, filtered and concentrated to dryness under reduced pressure. The residue was purified by flash chromatography on silica gel (petroleum ether:ethyl acetate=1:1) to afford methyl (2S,4R)-1-((4-phenoxybutanoyl)glycyl)-4-(p-tolyloxy)pyrrolidine-2-carboxylate (380 mg, yield 32.2%) as the white solid. LC/MS (ESI) m/z:455 (M+H)⁺.

### Step 4: (2S,4R)-1-((4-phenoxybutanoyl)glycyl)-4-(p-tolyloxy)pyrrolidine-2-carboxylic acid (5)

To a solution of methyl (2S,4R)-1-((4-phenoxybutanoyl)glycyl)-4-(p-tolyloxy)pyrrolidine-2-carboxylate (380 mg, 0.84 mmol) in THF/MeOH/H₂O (3 mL/2 mL/1 mL) was added LiOH (35 mg, 0.84 mmol) under N₂ atmosphere and the reaction mixture was stirred at room temperature for 3 hours. The mixture was diluted with ethyl acetate and washed with 1 N aq.HCl solution. The aqueous layer was extracted with ethyl acetate twice and the combined organic layers were washed with brine, dried over anhydrous Na₂SO₄, filtered and concentrated to dryness under reduced pressure to give (2S,4R)-1-((4-phenoxybutanoyl)glycyl)-4-(p-tolyloxy)pyrrolidine-2-carboxylic acid (360 mg, yield 97.8%) as white solid. LC/MS (ESI) m/z:441 (M+H)⁺.

### Step 5: (2S,4R)-N-((R)-1-(4-carbamimidoylthiophen-2-yl)ethyl)-1-((4-phenoxybutanoyl)glycyl)-4-(p-tolyloxy)pyrrolidine-2-carboxamide (Compound 131)

To a solution of (2S,4R)-1-((4-phenoxybutanoyl)glycyl)-4-(p-tolyloxy)pyrrolidine-2-carboxylic acid (50 mg, 0.11 mmol) in DMF (3 mL) was added DIPEA (73 mg, 0.57 mmol) and PyBOP (59 mg, 0.11 mmol) under N₂ atmosphere at 0° C. After stirring at room temperature for 15 minutes, (R)-5-(1-aminoethyl)thiophene-3-carboximidamide (19 mg, 0.11 mmol) was added to the above mixture under N₂ atmosphere and the resulting mixture was stirred at room temperature overnight. The mixture was diluted with ethyl acetate and washed with saturated aq.NaHCO₃ solution. The aqueous layer was extracted with ethyl acetate twice and the combined organic layers were washed with brine, dried over anhydrous Na₂SO₄, filtered and concentrated to dryness under reduced pressure. The residue was purified by prep-HPLC to give Compound 131 (12 mg, yield 17.9%) as white solid. ¹H NMR (400 MHz, CD₃OD) δ 8.22 (t, J=2.5 Hz, 1H), 7.55 (d, J=14.0 Hz, 1H), 7.23 (dd, J=11.2, 4.8 Hz, 2H), 7.10 (d, J=8.5 Hz, 2H), 6.90-6.87 (m, 3H), 6.82 (dd, J=8.5, 2.0 Hz, 2H), 5.35-5.23 (m, 1H), 5.02 (d, J=18.7 Hz, 1H), 4.58 (dd, J=16.7, 8.5 Hz, 1H), 4.00 (dd, J=13.3, 7.1 Hz, 3H), 3.93-3.84 (m, 2H), 3.16 (dd, J=6.6, 2.9 Hz, 1H), 2.54-2.43 (m, 3H), 2.26 (d, J=3.4 Hz, 3H), 2.19 (ddd, J=13.4, 8.8, 4.6 Hz, 1H), 2.09-2.04 (m, 2H), 1.60 (dd, J=23.4, 7.0 Hz, 3H). LC/MS (ESI) m/z: 592 (M+H)⁺.

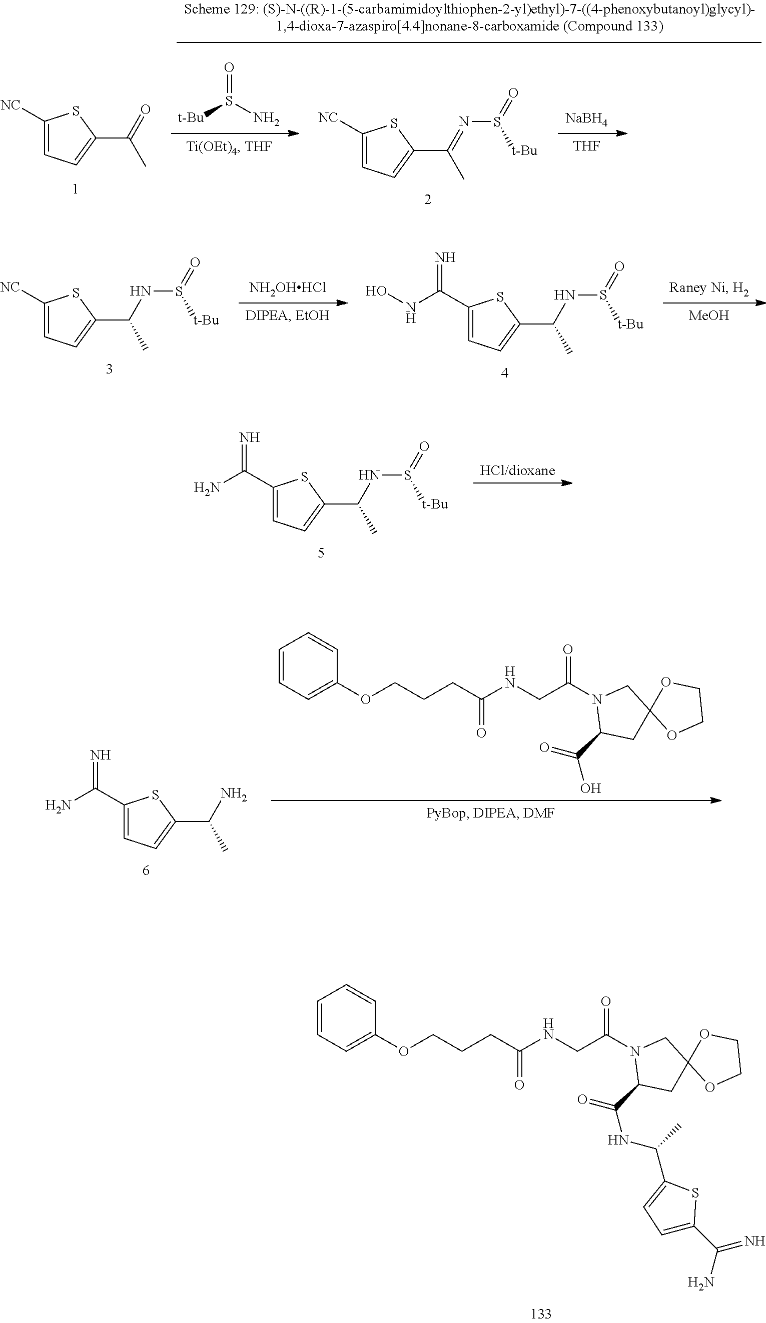

### Step 1: (R,L)-N-(1-(5-cyanothiophen-2-yl) ethylidene)-2-methylpropane-2-sulfonamide

To a solution of (R)-2-methylpropane-2-sulfinamide (898 mg, 7.41 mmol) in THE (10 mL) was added a mixture of 5-acetylthiophene-2-carbonitrile (933 mg, 6.17 mmol) in THE (10 mL) and Ti(OEt)₄ (4.91 g, 17.3 mmol) at 25° C. under N₂ atmosphere. The mixture was stirred at 70° C. for 16 hours. The mixture was poured into ice-cooled brine and filtered. The filter cake was washed with ethyl acetate twice. The filtrate was washed with brine, dried over anhydrous Na₂SO₄, filtered and concentrated to dryness under reduced pressure. The residue was purified by flash chromatography (petroleum ether:ethyl acetate=5:1) to give (R,E)-N-(1-(5-cyanothiophen-2-yl)ethylidene)-2-methylpropane-2-sulfinamide (760 mg, 48.7% yield) as yellow solid. LC/MS (ESI) (m/z): 255 (M+H)⁺.

### Step 2: (R)-N-((R)-1-(5-cyanothiophen-2-yl)ethyl)-2-methylpropane-2-sulfinamide

To a solution of (R,E)-N-(1-(5-cyanothiophen-2-yl)ethylidene)-2-methylpropane-2-sulfinamide (400 mg, 1.57 mmol) in THE (4 mL) was added NaBH₄ (90 mg, 2.36 mmol) in portions at −20° C. under N₂ atmosphere and the mixture was stirred at 25° C. for 1 hour. The mixture was quenched with saturated aq.NH₄Cl solution at 0° C. and extracted with DCM twice. The combined organic layers were washed with brine, dried over anhydrous Na₂SO₄, filtered and concentrated to dryness under reduced pressure. The residue was purified by flash chromatography (petroleum ether:ethyl acetate=1:1) and further purified by SFC to give (R)-N-((R)-1-(5-cyanothiophen-2-yl)ethyl)-2-methylpropane-2-sulfinamide (260 mg, 64.7% yield) as colorless oil. LC/MS (ESI) m/z: 257 (M+H)⁺.

### Step 3: 5-((R)-1-(((R)-tert-butylsulfinyl)amino)ethyl)-N-hydroxythiophene-2-carboximidamide

To a solution of (R)-N-((R)-1-(5-cyanothiophen-2-yl)ethyl)-2-methylpropane-2-sulfinamide (220 mg, 1.16 mmol) in EtOH (5 mL) was added NH₂OH·HCl (120 mg, 1.74 mmol) and DIPEA (449 mg, 3.48 mmol) and the mixture was stirred at 25° C. for 16 hours. The mixture was concentrated to dryness under reduced pressure and the residue was purified by flash chromatography (DCM:MeOH=10:1) to give 5-((R)-1-(((R)-tert-butylsulfinyl)amino)ethyl)-N-hydroxythiophene-2-carboximidamide (170 mg, 50.7% yield) as colorless oil. LC/MS (ESI) m/z: 290 (M+H)⁺.

### Step 4: 5-((R)-1-(((R)-tert-butylsulfinyl)amino)ethyl)thiophene-2-carboximidamide

To a solution of 5-((R)-1-(((R)-tert-butylsulfinyl)amino)ethyl)-N-hydroxythiophene-2-carboximidamide (170 mg, 0.58 mmol) in MeOH (3 mL) was added Raney Ni (20 mg) and acetic acid (0.1 mL) under N₂ atmosphere. The mixture was stirred under a H₂ balloon at 30° C. for 16 hours. The mixture was filtered and the filtrate was concentrated to dryness under reduced pressure to give 5-((R)-1-(((R)-tert-butylsulfinyl)amino)ethyl)thiophene-2-carboximidamide (80 mg, 50.6% yield) as light yellow solid, which was used directly in the next step without further purification. LC/MS (ESI) (m/z): 274 (M+H)⁺.

### Step 5: (R)-5-(1-aminoethyl)thiophene-2-carboximidamide

A solution of 5-((R)-1-(((R)-tert-butylsulfinyl)amino)ethyl)thiophene-2-carboximidamide (80 mg, 0.29 mmol) in HCl/1,4-dioxane (2 mL) was stirred at room temperature for 1 hour. The reaction mixture was concentrated to dryness under reduced pressure, co-evaporated with dichloromethane twice and dried under vacuum to give (R)-5-(1-aminoethyl)thiophene-2-carboximidamide (45 mg, 91.8% yield) as colorless oil, which was used directly in the next step without further purification. LC/MS (ESI) m/z: 170 (M+H)⁺.

### Step 6: (S)-N-((R)-1-(5-carbamimidoylthiophen-2-yl)ethyl)-7-((4-phenoxybutanoyl)glycyl)-1,4-dioxa-7-azaspiro[4.4]nonane-8-carboxamide (Compound 133)

To a mixture of (S)-7-((4-phenoxybutanoyl)glycyl)-1,4-dioxa-7-azaspiro[4.4]nonane-8-carboxylic acid (35 mg, 0.09 mmol) and (R)-5-(1-aminoethyl)thiophene-2-carboximidamide (26 mg, 0.13 mmol) in DMF (1 mL) was added DIPEA (23 mg, 0.18 mmol), PyBop (65 mg, 0.13 mmol) and 6-chloro-1H-benzo[d][1,2,3]triazol-1-ol (21 mg, 0.13 mmol) at 0° C. under N₂ atmosphere and the mixture was stirred at 25° C. for 16 hours. The mixture was diluted with water and extracted with CHCl₃/i-PrOH (3×10 mL, V/V=3/1). The combined organic layers were dried over anhydrous Na₂SO₄, filtered and concentrated to dryness under reduced pressure. The residue was purified by flash chromatography (DCM:MeOH=5:1) and further purified by prep-HPLC to give Compound 133 (4.6 mg, 9.4% yield) as white solid. ¹H NMR (400 MHz, CD₃OD) δ 7.79 (d, J=4.0 Hz, 1H), 7.26-7.21 (m, 3H), 6.89 (dd, J=6.1, 3.5 Hz, 3H), 5.29 (q, J=7.0 Hz, 1H), 4.57-4.48 (m, 1H), 3.99 (ddd, J=7.5, 6.5, 4.3 Hz, 8H), 3.70 (d, J=4.9 Hz, 2H), 2.47 (t, J=7.4 Hz, 2H), 2.40 (dd, J=13.4, 9.0 Hz, 1H), 2.18 (dd, J=13.3, 6.7 Hz, 1H), 2.11-2.05 (m, 2H), 1.60 (t, J=13.0 Hz, 3H). LC/MS (ESI) m/z: 544 (M+H)⁺.

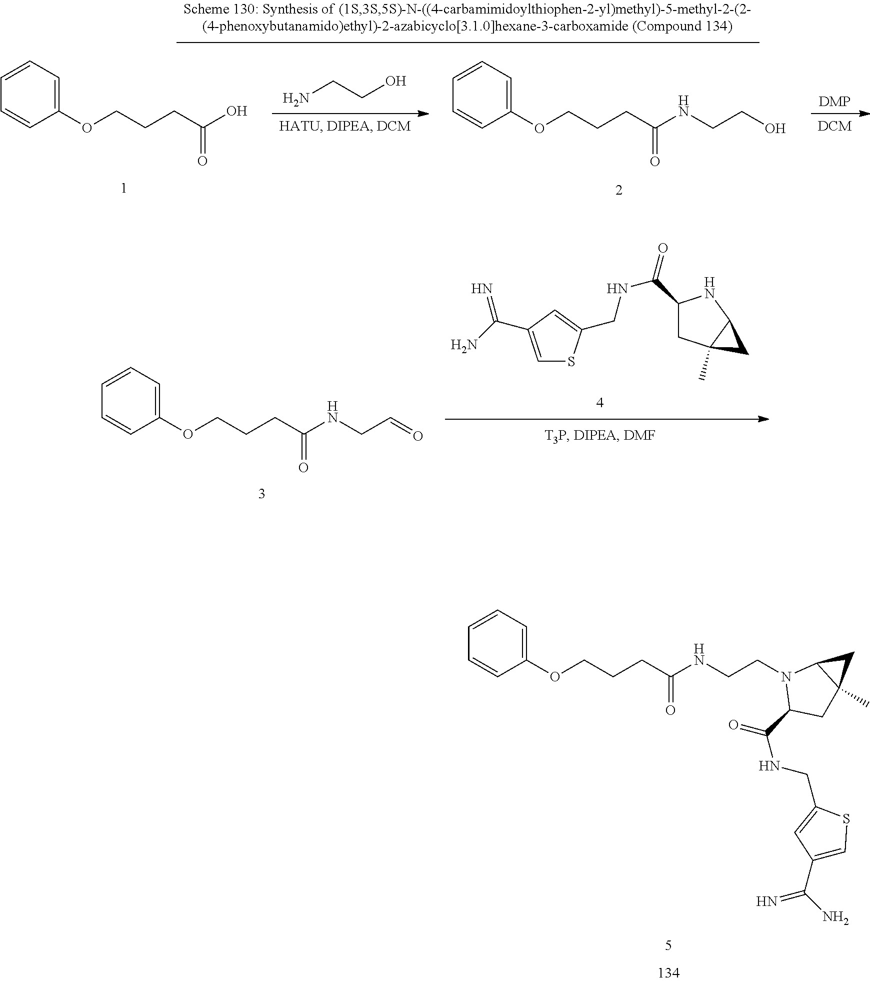

### Step 1: N-(2-hydroxyethyl)-4-phenoxybutanamide

To a mixture of 4-phenoxybutanoic acid (500 mg, 2.78 mmol) and 2-aminoethan-1-ol (61 mg, 3.33 mmol) in DMF (2 mL) was added DIPEA (1.79 g, 13.88 mmol) and HATU (1.58 g, 4.16 mmol) at 0° C. and the mixture was stirred under N₂ atmosphere at room temperature overnight. The mixture was diluted with ethyl acetate and washed with saturated aq. NaHCO₃ solution. The aqueous layer was extracted with ethyl acetate twice and the combined organic layers were washed with brine, dried over anhydrous Na₂SO₄, filtered and concentrated to dryness under reduced pressure. The residue was purified by flash chromatography on silica gel (petroleum ether:ethyl acetate=1:1) to give N-(2-hydroxyethyl)-4-phenoxybutanamide (350 mg, 56.3% yield) as white solid. LC/MS (ESI) (m/z): 224(M+H)⁺.

### Step 2: N-(2-oxoethyl)-4-phenoxybutanamide

To a mixture of N-(2-hydroxyethyl)-4-phenoxybutanamide (50 mg, 0.23 mmol) in DCM was added NaHCO₃ (38 mg, 0.49 mmol) and Dess-Martin periodinane (195 mg, 0.46 mmol) at 0° C. and the mixture was stirred at room temperature overnight. The mixture was quenched with 1N aq. Na₂S₂O₃ solution and extracted with DCM twice. The combined organic layers were washed with brine, dried over anhydrous Na₂SO₄, filtered and concentrated under reduced pressure to dryness. The residue was purified by flash chromatography on silica gel (PE:ethyl acetate=10:1) to give N-(2-oxoethyl)-4-phenoxybutanamide (20 mg, 40% yield) as white solid. LC/MS (ESI) (m/z): 222 (M+H)⁺.

### Step 3: (1S,3S,5S)-N-((4-carbamimidoylthiophen-2-yl)methyl)-5-methyl-2-(2-(4-phenoxybutanamido)ethyl)-2-azabicyclo[3.1.0]hexane-3-carboxamide (Compound 134)

A mixture of N-(2-oxoethyl)-4-phenoxybutanamide (13 mg, 0.06 mmol), (1S,3S,5S)-N-((4-carbamimidoylthiophen-2-yl)methyl)-5-methyl-2-azabicyclo[3.1.0]hexane-3-carboxamide (17 mg, 0.06 mmol) and 2 drops of acetic acid in MeOH (1 mL) was stirred at 0° C. for 30 min and NaBH₃CN (12 mg, 0.18 mmol) was added. The resulting mixture was stirred at 30° C. for 4 hours. The mixture was quenched with water and concentrated to dryness under reduced pressure. The residue was purified by prep-HPLC to give (1S,3S,5S)-N-((4-carbamimidoylthiophen-2-yl)methyl)-5-methyl-2-(2-(4-phenoxybutanamido)ethyl)-2-azabicyclo[3.1.0]hexane-3-carboxamide (1.0 mg, 3.5% yield) as off-white solid. ¹H NMR (400 MHz, CD₃OD) δ 8.25 (d, J=1.6 Hz, 1H), 7.44 (s, 1H), 7.23 (dd, J=8.7, 7.4 Hz, 2H), 6.92-6.86 (m, 3H), 4.82-4.80 (m, 1H), 4.59 (s, 2H), 3.99 (t, J=6.2 Hz, 2H), 3.64-3.57 (m, 1H), 3.46 (ddd, J=9.2, 4.6, 1.1 Hz, 2H), 3.33 (s, 2H), 2.42 (t, J=7.5 Hz, 2H), 2.37-2.28 (m, 1H), 2.07-2.01 (m, 3H), 1.60 (s, 1H), 1.30 (s, 3H), 0.86-0.75 (m, 1H). LC/MS (ESI) (m/z): 484 (M+H)⁺.

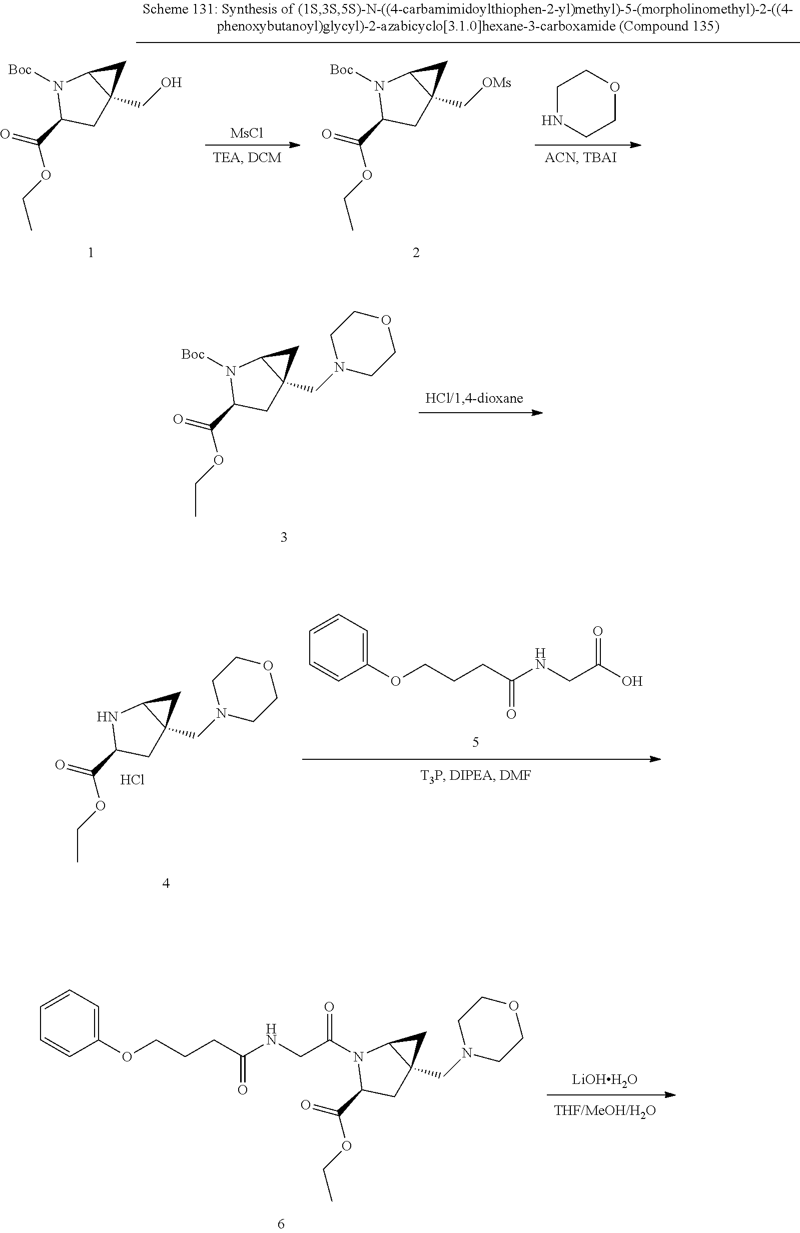

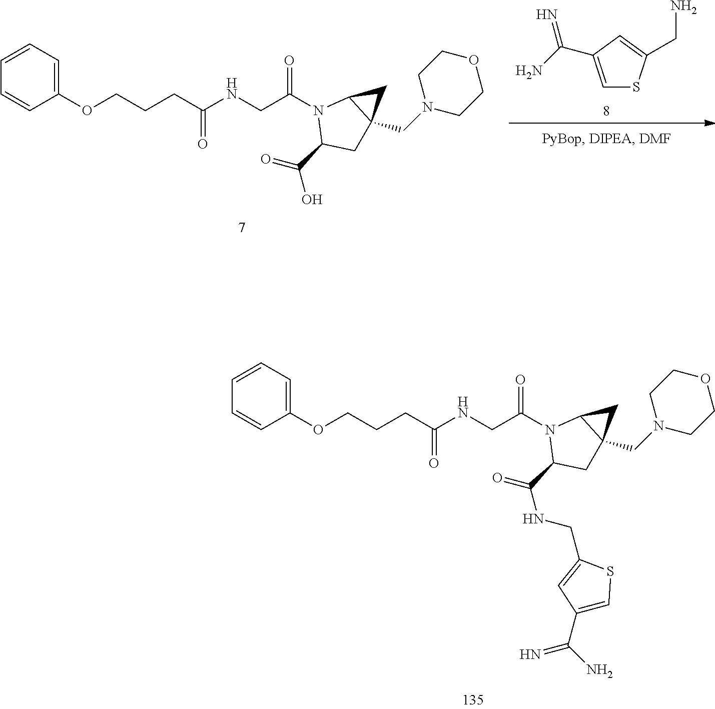

### Step 1: 2-(tert-butyl) 3-ethyl (1S,3S,5R)-5-(((methylsulfonyl)oxy)methyl)-2-azabicyclo[3.1.0]hexane-2,3-dicarboxylate (2)

To a solution of 2-(tert-butyl) 3-ethyl (1S,3S,5R)-5-(hydroxymethyl)-2-azabicyclo[3.1.0]hexane-2,3-dicarboxylate (300 mg, 1.05 mmol) in DCM (5.0 mL) was added TEA (1.44 mL, 10.5 mmol) and MsCl (0.42 mL, 5.25 mmol) at 0° C. and the mixture was stirred at 0° C. for 1.5 hours. The mixture was added water and extracted with DCM twice. The combined organic layers were washed with brine, dried over anhydrous Na₂SO₄, filtered and concentrated to dryness under reduced pressure to give 2-(tert-butyl) 3-ethyl (1 S,3S,5R)-5-(((methylsulfonyl)oxy)methyl)-2-azabicyclo[3.1.0]hexane-2,3-dicarboxylate (254 mg, 66.3% yield) as yellow oil, which was used directly in next step without further purification. LC/MS (ESI) m/z: 364 (M+H)⁺.

### Step 2: 2-(tert-butyl) 3-ethyl (1S,3S,5S)-5-(morpholinomethyl)-2-azabicyclo[3.1.0]hexane-2,3-dicarboxylate (3)

To a mixture of 2-(tert-butyl) 3-ethyl (1 S,3S,5R)-5-(((methylsulfonyl)oxy)methyl)-2-azabicyclo[3.1.0]hexane-2,3-dicarboxylate (254 mg, 0.70 mmol) and morpholine (0.12 mL, 1.40 mmol) in ACN (5 mL) was added TBAI (129 mg, 0.35 mmol) and the mixture was stirred at 50° C. for 16 hours. The mixture was purified by column chromatography on silica gel (DCM:MeOH=10:1) to give 2-(tert-butyl) 3-ethyl (1 S,3S,5S)-5-(morpholinomethyl)-2-azabicyclo[3.1.0]hexane-2,3-dicarboxylate (60 mg, 24.2% yield) as brown oil. LC/MS (ESI) m/z: 355 (M+H)⁺.

### Step 3: ethyl (1S,3S,5S)-5-(morpholinomethyl)-2-azabicyclo[3.1.0]hexane-3-carboxylate hydrochloride (4)

A solution of 2-(tert-butyl) 3-ethyl (1 S,3S,5S)-5-(morpholinomethyl)-2-azabicyclo[3.1.0]hexane-2,3-dicarboxylate (60 mg, 0.17 mmol) in HCl/1,4-dioxane (2 mL, 4M) was stirred at 25° C. for 2 hours. The reaction mixture was concentrated to dryness under reduced pressure, co-evaporated with DCM twice and dried under vacuum to give ethyl (1 S,3S,5S)-5-(morpholinomethyl)-2-azabicyclo[3.1.0]hexane-3-carboxylate (50 mg, 100% yield) as yellow solid, which was used directly in the next step without further purification. LC/MS (ESI) m/z: 255 (M+H)⁺.

### Step 4: ethyl (1S,3S,5S)-5-(morpholinomethyl)-2-((4-phenoxybutanoyl)glycyl)-2-azabicyclo[3.1.0]hexane-3-carboxylate (6)

To a mixture of ethyl (1 S,3S,5S)-5-(morpholinomethyl)-2-azabicyclo[3.1.0]hexane-3-carboxylate (50 mg, 0.21 mmol) and (4-phenoxybutanoyl)glycine (50 mg, 0.21 mmol) in DMF (3.0 mL) was added DIPEA (0.20 mL, 1.26 mmol) and T₃P (396 mg, 0.63 mmol, 50% wt in ethyl acetate) at 0° C. under N₂ atmosphere and the mixture was stirred at 25° C. for 16 hours. The mixture was diluted with water and extracted with ethyl acetate twice. The combined organic layers were dried over anhydrous Na₂SO₄, filtered and concentrated to dryness under reduced pressure. The residue was purified by flash column chromatography on silica gel (DCM:MeOH=20:1) to give ethyl (1S,3S,5S)-5-(morpholinomethyl)-2-((4-phenoxybutanoyl)glycyl)-2-azabicyclo[3.1.0]hexane-3-carboxylate (30 mg, 30% yield) as yellow solid. LC/MS (ESI) m/z: 474 (M+H)⁺.

### Step 5: (1S,3S,5S)-5-(morpholinomethyl)-2-((4-phenoxybutanoyl)glycyl)-2-azabicyclo[3.1.0]hexane-3-carboxylic acid (7)

To a solution of ethyl (1 S,3S,5S)-5-(morpholinomethyl)-2-((4-phenoxybutanoyl)glycyl)-2-azabicyclo[3.1.0]hexane-3-carboxylate (30 mg, 0.06 mmol) in MeOH (2 mL), THE (1 mL) and water (1 mL) was added lithium hydroxide (8 mg, 0.09 mmol) at 0° C. The mixture was stirred at room temperature for 4 hours. The aqueous layer was acidified with 1N aq.HCl to pH=3, and extracted with DCM twice. The combined organic layers were washed with brine, dried over Na₂SO₄, filtered and concentrated to dryness under reduced pressure to give (1 S,3S,5S)-5-(morpholinomethyl)-2-((4-phenoxybutanoyl)glycyl)-2-azabicyclo[3.1.0]hexane-3-carboxylic acid (25 mg, 89.3% yield) as light yellow solid, which was used directly in next step without further purification. LC/MS (ESI) (m/z): 446 (M+H)⁺.

### Step 6: (1S,3S,5S)-N-((4-carbamimidoylthiophen-2-yl)methyl)-5-(morpholinomethyl)-2-((4-phenoxybutanoyl)glycyl)-2-azabicyclo[3.1.0]hexane-3-carboxamide (Compound 135)

To a mixture of (1 S,3S,5S)-5-(morpholinomethyl)-2-((4-phenoxybutanoyl)glycyl)-2-azabicyclo[3.1.0]hexane-3-carboxylic acid (30 mg, 0.07 mmol) and 5-(aminomethyl) thiophene-3-carboximidamide (21 mg, 0.13 mmol) in DMF (3 mL) was added PyBOP (52 mg, 0.11 mmol) and the mixture was stirred at 25° C. for 3 hours. The mixture was concentrated to dryness under reduced pressure. The residue was purified by TLC (DCM:MeOH=10:1) and prep-HPLC to give Compound 135 (2.0 mg, yield 5.1%) as white solid. ¹H NMR (400 MHz, CD₃OD) δ 8.55 (s, 1H), 8.22 (d, J=1.6 Hz, 1H), 7.87-7.82 (m, 2H), 7.50 (s, 1H), 7.45-7.40 (m, 2H), 7.21 (t, J=7.4 Hz, 1H), 7.08-7.04 (m, 2H), 7.03-6.99 (m, 2H), 5.23 (q, J=6.6 Hz, 1H), 4.41 (d, J=16.4 Hz, 1H), 4.26 (d, J=16.5 Hz, 1H), 3.59 (dd, J=6.8, 3.3 Hz, 2H), 3.53 (t, J=5.6 Hz, 2H), 3.46 (d, J=10.5 Hz, 1H), 3.37 (d, J=4.3 Hz, 2H), 2.69 (dd, J=15.0, 9.7 Hz, 1H), 2.11 (dd, J=13.4, 3.7 Hz, 1H), 1.95 (d, J=2.6 Hz, 3H), 1.91 (s, 1H), 1.58 (d, J=7.0 Hz, 3H), 1.28 (dd, J=5.8, 2.8 Hz, 1H), 0.98 (t, J=5.8 Hz, 1H). LC/MS (ESI) m/z: 583 (M+H)⁺.

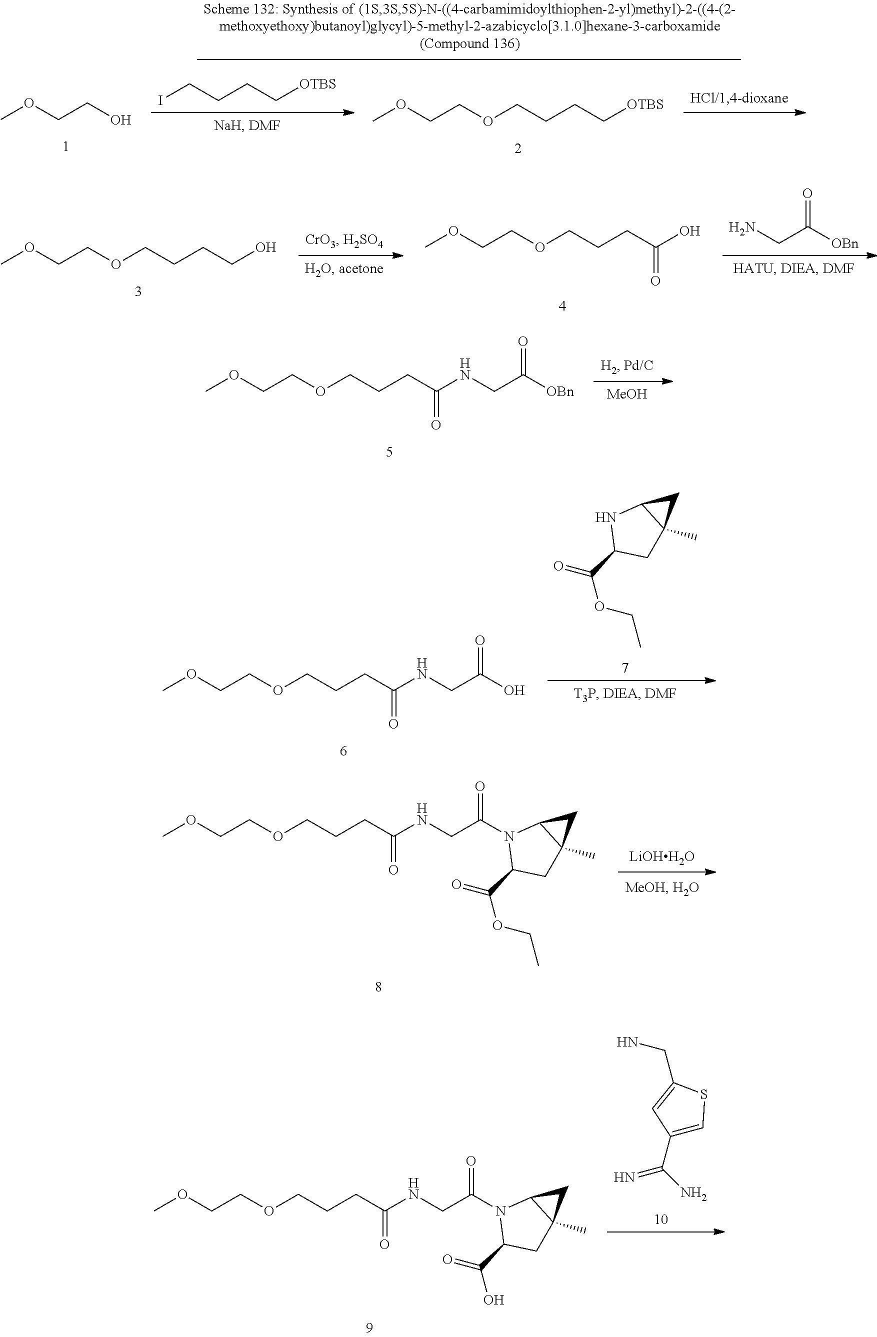

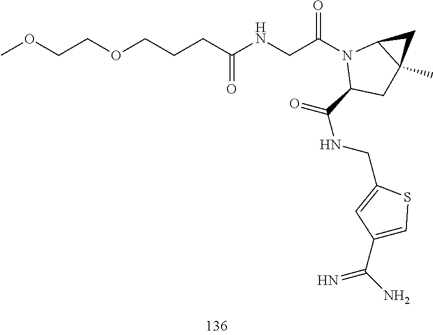

### Step 1: 11,11,12,12-tetramethyl-2,5,10-trioxa-11-silatridecane

To a mixture of benzyl (2S,4S)-2-(4-bromo-2-hydroxyphenyl)-4-hydroxypiperidine-1-carboxylate (2 g, 4.92 mmol) and 2-methoxyethanol (58 mg, 7.62 mmol) in DMF (6 mL) was added NaH (760 mg, 19 mmol, 60% dispersion in mineral oil) in portions at 0° C. and the mixture was stirred at 0° C. for half an hour. Tert-butyl(4-iodobutoxy)dimethylsilane (2.0 g, 6.35 mmol) was added to the mixture and the mixture was stirred at 25° C. for 2 hours. The mixture was quenched with sat.NH₄Cl solution at 0° C. and extracted with EtOAc twice. The combined organic layers were washed with brine, dried over anhydrous Na₂SO₄, filtered and concentrated to dryness under vacuum to give 11,11,12,12-tetramethyl-2,5,10-trioxa-11-silatridecane (0.5 g, 60.1% yield) as colorless oil, which was used directly in next step without further purification.

### Step 2: 4-(2-methoxyethoxy)butan-1-ol

A solution of 11,11,12,12-tetramethyl-2,5,10-trioxa-11-silatridecane (1 g, 3.81 mmol) in HCl/1,4-dioxane (15 mL, 4M) was stirred at room temperature for half an hour. The reaction mixture was concentrated to dryness under vacuum to give 4-(2-methoxyethoxy)butan-1-ol (0.5 g, 88.5% yield) as yellow oil, which was used directly in the next step without further purification.

### Step 3: 4-(2-methoxyethoxy)butanoic acid

To a solution of 4-(2-methoxyethoxy)butan-1-ol (0.5 g, 3.37 mmol) in acetone (20 mL) was added a solution of CrO₃ (2.7 g, 27 mmol) in water (7 mL) and H₂SO₄ (2.3 mL) drop-wisely at 0° C. and the mixture was stirred at room temperature for 2 hours. The reaction was quenched with isopropyl alcohol (10 mL), diluted with water (20 mL) and extracted with ethyl acetate (3×20 mL). The combined organic layer was washed with brine (2×20 mL), dried over anhydrous Na₂SO₄, filtered and concentrated to dryness under reduced pressure to give 4-(2-methoxyethoxy)butanoic acid (500 mg, 91% yield) as a yellow oil. LC/MS (ESI) m/z: 161 (M−H)⁻.

### Step 4: benzyl (4-(2-methoxyethoxy)butanoyl)glycinate

To a mixture of 4-(2-methoxyethoxy)butanoic acid (400 mg, 2.46 mmol), benzyl 2-aminoacetate (428 mg, 2.59 mmol) and HATU (1.4 g, 3.69 mmol) in DMF (4 mL) was added DIEA (954 mg, 7.40 mmol) and the mixture was stirred under N₂ atmosphere at room temperature for 1 hour. The mixture was diluted with water (20 mL) and extracted with ethyl acetate (3×20 mL). The combined organic layers were washed with brine, dried over anhydrous Na₂SO₄, filtered and concentrated to dryness under reduced pressure. The residue was further purified by prep-TLC (PE:EtOAc=3:1) to give benzyl 2-[4-(2-methoxyethoxy)butanamido]acetate (0.5 g, 65% yield) as yellow oil.

### Step 5: (4-(2-methoxyethoxy)butanoyl)glycine

To a solution of benzyl 2-[4-(2-methoxyethoxy)butanamido]acetate (90 mg, 0.29 mmol) in MeOH (5 mL) was added Pd/C (10 mg, 10% wt) at 25° C. The mixture was degassed under N₂ atmosphere for three times and stirred under a H₂ balloon at 20° C. for 1 hour. The mixture was filtered and the filtrate was concentrated to dryness under reduced pressure to give [4-(2-methoxyethoxy)butanamido]acetic acid (65 mg, 81% yield) as yellow oil. LC/MS (ESI) m/z: 220 (M+H)⁺.

### Step 6: ethyl (1S,3S,5S)-2-((4-(2-methoxyethoxy)butanoyl)glycyl)-5-methyl-2-azabicyclo[3.1.0]hexane-3-carboxylate

To a mixture of [4-(2-methoxyethoxy)butanamido]acetic acid (60 mg, 0.27 mmol) and ethyl (1S,3S,5S)-5-methyl-2-azabicyclo[3.1.0]hexane-3-carboxylate (51 mg, 0.30 mmol) in DMF (3 mL) was added DIEA (212 mg, 1.64 mmol) followed by T₃P (522 mg, 0.82 mmol, 50% wt in ethyl acetate) at 0° C., and the mixture was stirred at 25° C. for 1 hour. The mixture was diluted with water and extracted with ethyl acetate three times. The combined organic layers were washed with brine, dried over anhydrous Na₂SO₄, filtered and concentrated to dryness under reduced pressure to give ethyl (1S,3S,5S)-2-{2-[4-(2-methoxyethoxy)butanamido]acetyl}-5-methyl-2-azabicyclo[3.1.0]hexane-3-carboxylate (0.1 g, 98.3% yield) as yellow oil. LC/MS (ESI) m/z: 371 (M+H)⁺.

### Step 7: (1S,3S,5S)-2-((4-(2-methoxyethoxy)butanoyl)glycyl)-5-methyl-2-azabicyclo[3.1.0]hexane-3-carboxylic acid

To a mixture of ethyl (1 S,3S,5S)-2-{2-[4-(2-methoxyethoxy)butanamido]acetyl}-5-methyl-2-azabicyclo[3.1.0]hexane-3-carboxylate (100 mg, 0.27 mmol) and LiOH·H₂O (34 mg, 0.81 mmol) in MeOH (2 mL) and water (1 mL) was stirred at 25° C. for 1 hour. The mixture was acidified with 0.5M HCl to pH-3 and concentrated to dryness under vacuum to give (1 S,3S,5S)-2-{2-[4-(2-methoxyethoxy)butanamido]acetyl}-5-methyl-2-azabicyclo[3.1.0]hexane-3-carboxylic acid (100 mg, quantitative) as white solid.

### Step 8: (1S,3S,5S)-N-((4-carbamimidoylthiophen-2-yl)methyl)-2-((4-(2-methoxyethoxy) butanoyl)glycyl)-5-methyl-2-azabicyclo[3.1.0]hexane-3-carboxamide (Compound 136)

To a mixture of (1 S,3S,5S)-2-{2-[4-(2-methoxyethoxy)butanamido]acetyl}-5-methyl-2-azabicyclo[3.1.0]hexane-3-carboxylic acid (100 mg, 0.29 mmol) and 5-(aminomethyl) thiophene-3-carboximidamide (45 mg, 0.29 mmol) in DMF (2 mL) was added DIEA (113 mg, 0.87 mmol) followed by PyBOP (243 mg, 0.46 mmol) at 0° C. and the mixture was stirred at 25° C. for 1 hour. The resulting mixture was diluted with water (10 mL) and extracted with ethyl acetate (3×10 mL). The combined organic layers were washed with brine (2×10 mL), dried over anhydrous Na₂SO₄, filtered and concentrated to dryness under reduced pressure. The residue was further purified by prep-HPLC to afford Compound 136 (0.5 mg, yield 0.35%) as white solid. ¹H NMR (400 MHz, CD₃OD) δ 8.52 (s, 1H), 8.23 (d, J=1.6 Hz, 1H), 7.42 (s, 1H), 5.18 (s, 1H), 4.79 (dd, J=11.5, 3.4 Hz, 4H), 4.53 (d, J=12.5 Hz, 2H), 4.15 (q, J=16.7 Hz, 2H), 3.58-3.53 (m, 4H), 3.49 (d, J=6.2 Hz, 2H), 3.36 (s, 3H), 2.36 (dd, J=16.1, 8.9 Hz, 3H), 2.15 (dd, J=13.4, 3.4 Hz, 1H), 1.90-1.82 (m, 2H), 1.13 (dd, J=5.8, 2.5 Hz, 1H), 0.81 (t, J=5.1 Hz, 1H). LC/MS (ESI) m/z: 480 (M+H)⁺.

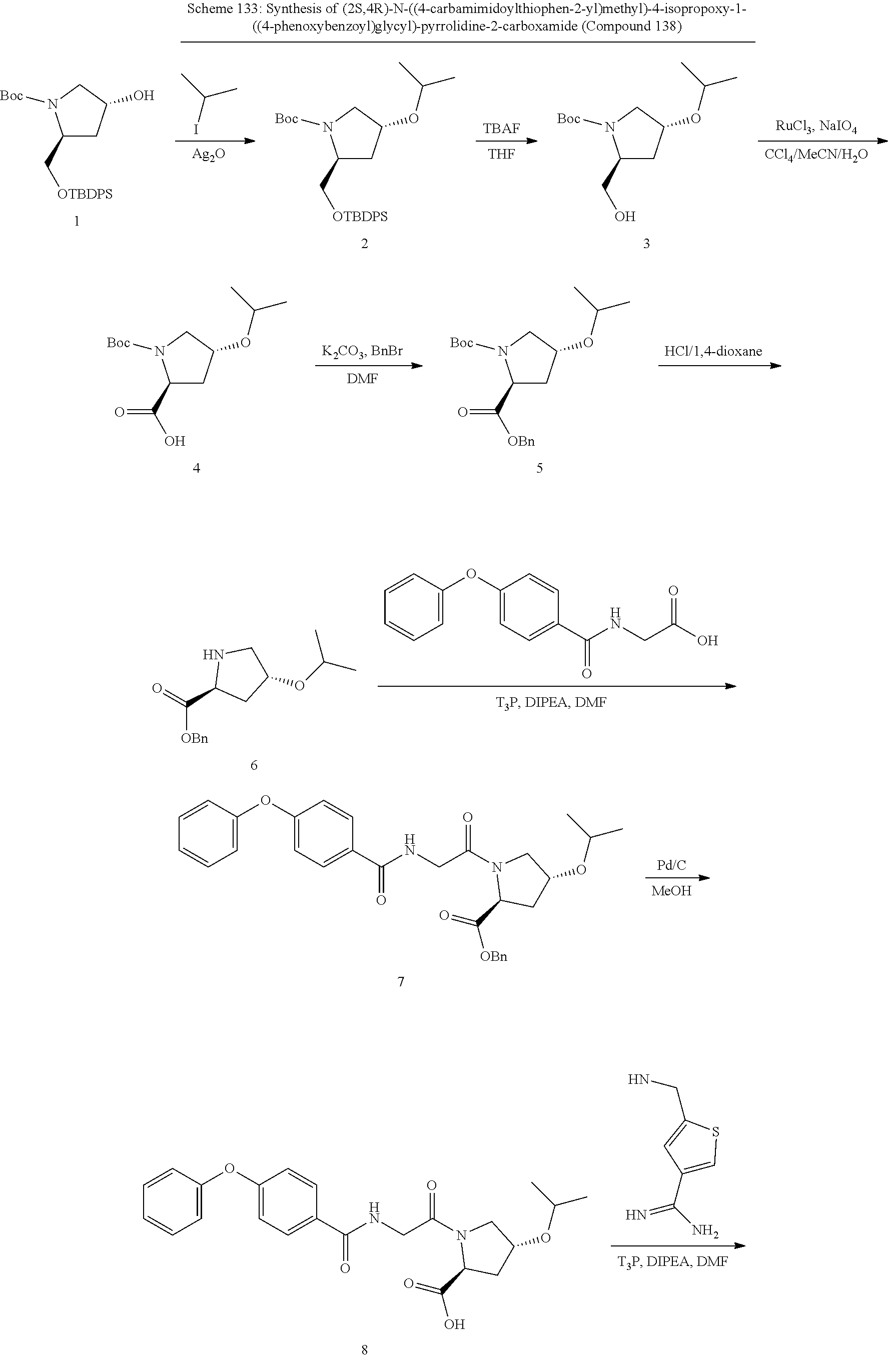

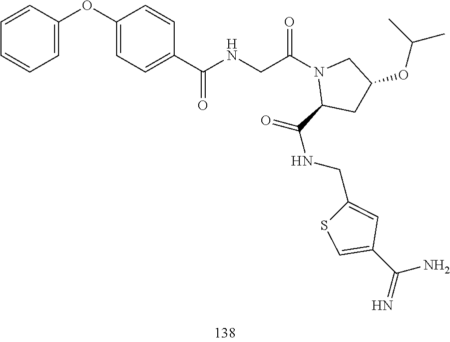 [SMILES: CC(C)O[C@@H]1C[C@@H](C(=O)NCc2cc(C(=N)N)cs2)N(C(=O)CNC(=O)c2ccc(Oc3ccccc3)cc2)C1]

### Step 1: tert-butyl (2S,4R)-2-(((tert-butyidiphenyisilyl)oxy)methyl)-4-isopropoxypyrrolidine-1-carboxylate (2)

To a solution of tert-butyl (2S,4R)-2-(((tert-butyidiphenyisilyl)oxy)methyl)-4-hydroxypyrrolidine-1-carboxylate (1 g, 2.20 mmol) in 2-iodopropane (6 mL) was added silver oxide (763 mg, 3.29 mmol). The mixture was heated up to 60° C. under N₂ atmosphere and stirred at 60° C. overnight. The mixture was diluted with ethyl acetate and washed with water. The aqueous layer was extracted with ethyl acetate twice and the combined organic layers were washed with brine, dried over anhydrous Na₂SO₄, filtered and concentrated to dryness under reduced pressure. The residue was purified by flash chromatography on silica gel (petroleum ether:ethyl acetate=5:1) to afford the title tert-butyl (2S,4R)-2-(((tert-butyldiphenylsilyl)oxy)methyl)-4-isopropoxypyrrolidine-1-carboxylate (270 mg, 24.7% Yield) as white solid. LC/MS (ESI) m/z:498 (M+H)⁺.

### Step 2: tert-butyl (2S,4R)-2-(hydroxymethyl)-4-isopropoxypyrrolidine-1-carboxylate (3)

To a solution of tert-butyl (2S,4R)-2-(((tert-butyldiphenylsilyl)oxy)methyl)-4-isopropoxypyrrolidine-1-carboxylate (270 mg, 0.54 mmol) in THE (3 mL) was added TBAF (425 mg, 1.63 mmol) under N₂ atmosphere and the reaction mixture was stirred at room temperature overnight. The mixture was diluted with ethyl acetate and washed with saturated aq.NH₄Cl solution. The aqueous layer was extracted with ethyl acetate and the combined organic layers were washed with brine, dried over anhydrous Na₂SO₄, filtered and concentrated to dryness under reduced pressure. The residue was purified by flash chromatography on silica gel (petroleum ether:ethyl acetate=5:1) to afford the title tert-butyl (2S,4R)-2-(hydroxymethyl)-4-isopropoxypyrrolidine-1-carboxylate (110 mg, Yield 78.2%) as white solid. LC/MS (ESI) m/z:260 (M+H)⁺.

### Step 3: (2S,4R)-1-(tert-butoxycarbonyl)-4-isopropoxypyrrolidine-2-carboxylic acid (4)

To a solution of tert-butyl (2S,4R)-2-(hydroxymethyl)-4-isopropoxypyrrolidine-1-carboxylate (110 mg, 0.42 mmol) in CCl₄:MeCN:H₂O=1:1:1.5 (3.5 mL) was added NaIO₄ (363 mg, 1.70 mmol) under N₂ atmosphere. After stirred at room temperature for 30 minutes, RuCl₃ (2 mg, 0.008 mmol) was added into the above mixture and the reaction mixture was stirred at room temperature for 2 hours. The mixture was diluted with ethyl acetate and washed with water. The aqueous layer was extracted with ethyl acetate twice and the combined organic layers were washed with brine, dried over anhydrous Na₂SO₄, filtered and concentrated to dryness under reduced pressure. The residue was purified by flash chromatography on silica gel (petroleum ether:ethyl acetate=5:1) to afford the title (2S,4R)-1-(tert-butoxycarbonyl)-4-isopropoxypyrrolidine-2-carboxylic acid (80 mg, Yield 69.0%) as white solid. LC/MS (ESI) m/z: 274 (M+H)⁺.

### Step 4: 2-benzyl 1-(tert-butyl) (2S,4R)-4-isopropoxypyrrolidine-1,2-dicarboxylate (5)

To a solution of (2S,4R)-1-(tert-butoxycarbonyl)-4-isopropoxypyrrolidine-2-carboxylic acid (80 mg, 0.29 mmol) in DMF (3 mL) was added Cs₂CO₃ (105 mg, 0.32 mmol) and benzyl bromide (50 mg, 0.29 mmol) under N₂ atmosphere and the reaction mixture was stirred at room temperature overnight. The mixture was diluted with ethyl acetate and washed with water. The aqueous layer was extracted with ethyl acetate twice and the combined organic layers were washed with brine, dried over anhydrous Na₂SO₄, filtered and concentrated to dryness under reduced pressure. The residue was purified by flash chromatography on silica gel (petroleum ether:ethyl acetate=5:1) to afford the title 2-benzyl 1-(tert-butyl) (2S,4R)-4-isopropoxypyrrolidine-1,2-dicarboxylate (70 mg, 0.19 mmol, Yield 65.8%) as white solid. LC/MS (ESI) m/z:364 (M+H)⁺.

### Step 5: benzyl (2S,4R)-4-isopropoxypyrrolidine-2-carboxylate (6)

To a solution of 2-benzyl 1-(tert-butyl) (2S,4R)-4-isopropoxypyrrolidine-1,2-dicarboxylate (70 mg, 0.19 mmol) in 1,4-dioxane (1 mL) was added HCl/1,4-dioxane (1 mL) under N₂ atmosphere and the reaction mixture was stirred at room temperature for 2 hours. The mixture was concentrated to dryness under reduced pressure to afford the title benzyl (2S,4R)-4-isopropoxypyrrolidine-2-carboxylate (50 mg, Yield 98.6%) as white solid. LC/MS (ESI) m/z:264 (M+H)⁺.

### Step 6: benzyl (2S,4R)-4-isopropoxy-1-((4-phenoxybenzoyl)glycyl)pyrrolidine-2-carboxylate (7)

To a solution of benzyl (2S,4R)-4-isopropoxypyrrolidine-2-carboxylate (50 mg, 0.19 mmol) and (4-phenoxybenzoyl)glycine (52 mg, 0.19 mmol) in DMF (5 mL) was added DIPEA (147 mg, 1.14 mmol) and T₃P (362 mg, 0.57 mmol) under N₂ atmosphere at 0° C. The reaction mixture was stirred at room temperature overnight. The mixture was diluted with ethyl acetate and washed with saturated aq.NaHCO₃ solution. The aqueous layer was extracted with ethyl acetate twice and the combined organic layers were washed with brine, dried over anhydrous Na₂SO₄, filtered and concentrated to dryness under reduced pressure. The residue was purified by flash chromatography on silica gel (petroleum ether:ethyl acetate=5:1) to afford the title benzyl (2S,4R)-4-isopropoxy-1-((4-phenoxybenzoyl)glycyl)pyrrolidine-2-carboxylate (80 mg, Yield 81.6%) as white solid. LC/MS (ESI) m/z:517 (M+H).

### Step 7: (2S,4R)-4-isopropoxy-1-((4-phenoxybenzoyl)glycyl)pyrrolidine-2-carboxylic acid (8)

To a solution of benzyl (2S,4R)-4-isopropoxy-1-((4-phenoxybenzoyl)glycyl)pyrrolidine-2-carboxylate (80 mg, 0.16 mmol) in methanol (3 mL) was added Pd/C (100 mg) under N₂ atmosphere and the reaction mixture was stirred at room temperature under a H₂ balloon for 3 hours. The mixture was filtered and the filtrate was concentrated to dryness under reduced pressure to afford the title (2S,4R)-4-isopropoxy-1-((4-phenoxybenzoyl)glycyl)pyrrolidine-2-carboxylic acid (50 mg, Yield 75.7%) as white solid. LC/MS (ESI) m/z:427 (M+H).

### Step 8: (2S,4R)-N-((4-carbamimidoylthiophen-2-yl)methyl)-4-isopropoxy-1-((4-phenoxybenzoyl)glycyl)pyrrolidine-2-carboxamide (Compound 138)

To a solution of (2S,4R)-4-isopropoxy-1-((4-phenoxybenzoyl)glycyl)pyrrolidine-2-carboxylic acid (30 mg, 0.07 mmol) and 5-(aminomethyl)thiophene-3-carboximidamide (11 mg, 0.07 mmol) in DMF (3 mL) was added DIPEA (55 mg, 0.42 mmol) and T₃P (134 mg, 0.21 mmol) under N₂ atmosphere at 0° C. The reaction mixture was stirred at room temperature overnight. The mixture was diluted with ethyl acetate and washed with saturated aq.NaHCO₃ solution. The aqueous layer was extracted with ethyl acetate and the combined organic layers were washed with brine, dried over anhydrous Na₂SO₄, filtered and concentrated to dryness under reduced pressure. The residue was purified by prep-HPLC to give the title Compound 138 (1.6 mg, Yield 4.0%) as white solid. ¹H NMR (400 MHz, CD₃OD) δ 8.23 (dd, J=17.4, 1.5 Hz, 1H), 7.84 (d, J=8.8 Hz, 2H), 7.52-7.38 (m, 3H), 7.21 (t, J=7.4 Hz, 1H), 7.03 (ddd, J=8.8, 7.7, 1.5 Hz, 4H), 4.57 (s, 2H), 4.51 (t, J=7.9 Hz, 1H), 4.40-4.30 (m, 1H), 4.23 (d, J=23.3 Hz, 2H), 3.84 (dd, J=10.9, 4.7 Hz, 1H), 3.72 (ddd, J=20.8, 12.1, 4.2 Hz, 2H), 2.33-2.18 (m, 1H), 2.10-2.03 (m, 1H), 1.17 (d, J=6.2 Hz, 6H). LC/MS (ESI) m/z:564 (M+H)⁺.

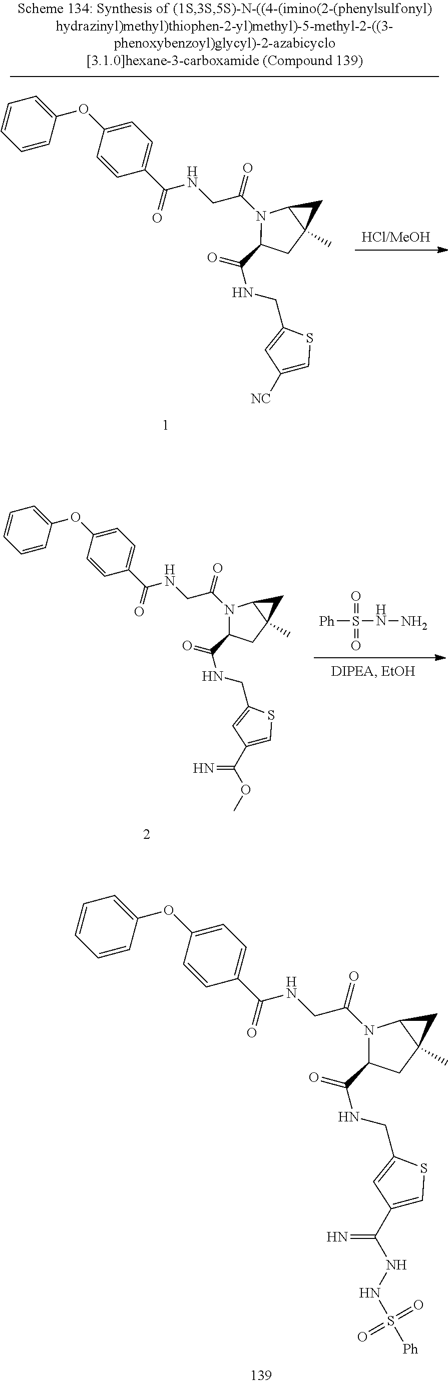

### Step 1: methyl 5-(((1S,3S,5S)-5-methyl-2-((4-phenoxybenzoyl)glycyl)-2-azabicyclo[3.1.0]hexane-3-carboxamido)methyl)thiophene-3-carbimidate (2)

(1S,3S,5S)-N-((4-cyanothiophen-2-yl)methyl)-5-methyl-2-((4-phenoxybenzoyl)glycyl)-2-azabicyclo[3.1.0]hexane-3-carboxamide (45 mg, 0.087 mmol) was dissolved in saturated HCl/MeOH (1 mL) at room temperature under N₂ atmosphere and the mixture was stirred at 25° C. for 2 hours. The reaction was concentrated to dryness under reduced pressure to give methyl 5-(((1 S,3S,5S)-5-methyl-2-((3-phenoxybenzoyl)glycyl)-2-azabicyclo[3.1.0]hexane-3-carboxamido)methyl)thiophene-3-carbimidate (40 mg, yield 84.1%) as colorless oil which was used directly in the next step without further purification. LC/MS (ESI) m/z: 547 (M+H)⁺.

### Step 2: (1S,3S,5S)-N-((4-(imino(2-(phenylsulfonyl)hydrazineyl)methyl)thiophen-2-yl)methyl)-5-methyl-2-((4-phenoxybenzoyl)glycyl)-2-azabicyclo[3.1.0]hexane-3-carboxamide (Compound 139)

A mixture of methyl 5-(((1 S,3S,5S)-5-methyl-2-((4-phenoxybenzoyl)glycyl)-2-azabicyclo[3.1.0]hexane-3-carboxamido)methyl)thiophene-3-carbimidate (40 mg, 0.073 mmol), benzenesulfonohydrazide (15 mg, 0.087 mmol) and DIPEA(28 mg, 0.22 mmol) in EtOH (1 mL) was stirred at 80° C. for 2 hours. The reaction solution was concentrated to dryness under reduced pressure. The residue was purified by prep-HPLC to give Compound 139 (1.6 mg, yield 3.2%) as white solid. ¹H NMR (400 MHz, CD₃OD) δ 7.94 (d, J=7.2 Hz, 1H), 7.86-7.83 (m, 2H), 7.76 (d, J=8.2 Hz, 1H), 7.63 (t, J=7.3 Hz, 1H), 7.56 (t, J=7.4 Hz, 2H), 7.42 (dd, J=8.5, 7.6 Hz, 2H), 7.26 (s, 1H), 7.21 (d, J=8.3 Hz, 2H), 7.10-7.03 (m, 2H), 7.03-6.97 (m, 2H), 4.80 (s, 1H), 4.50 (d, J=7.2 Hz, 2H), 4.37 (s, 1H), 4.28 (d, J=16.5 Hz, 1H), 2.20-2.17 (m, 2H), 2.05-2.01 (m, 3H), 1.13 (d, J=3.2 Hz, 1H), 0.76 (d, J=7.3 Hz, 1H). LC/MS (ESI) m/z: 687 (M+H)⁺.

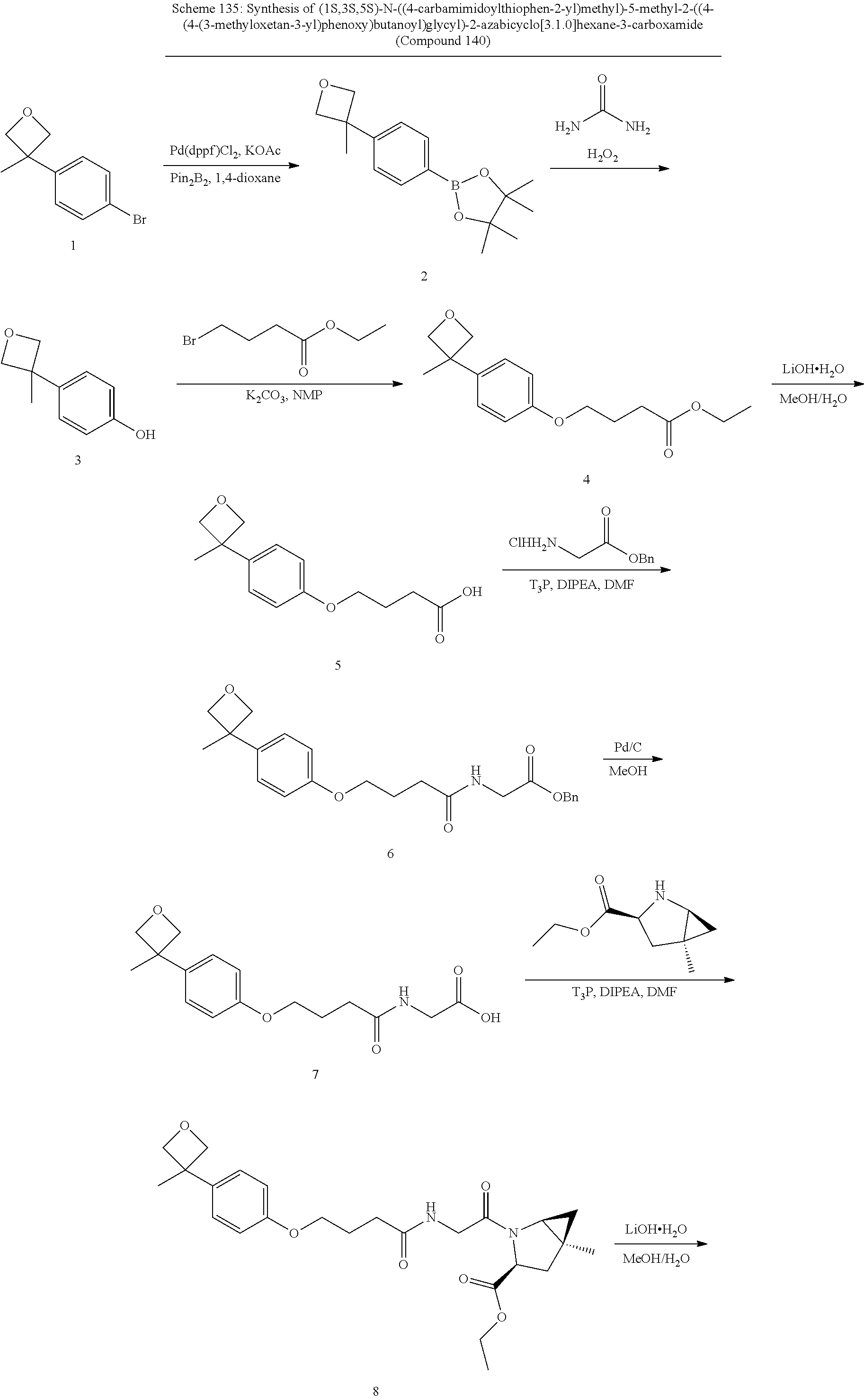

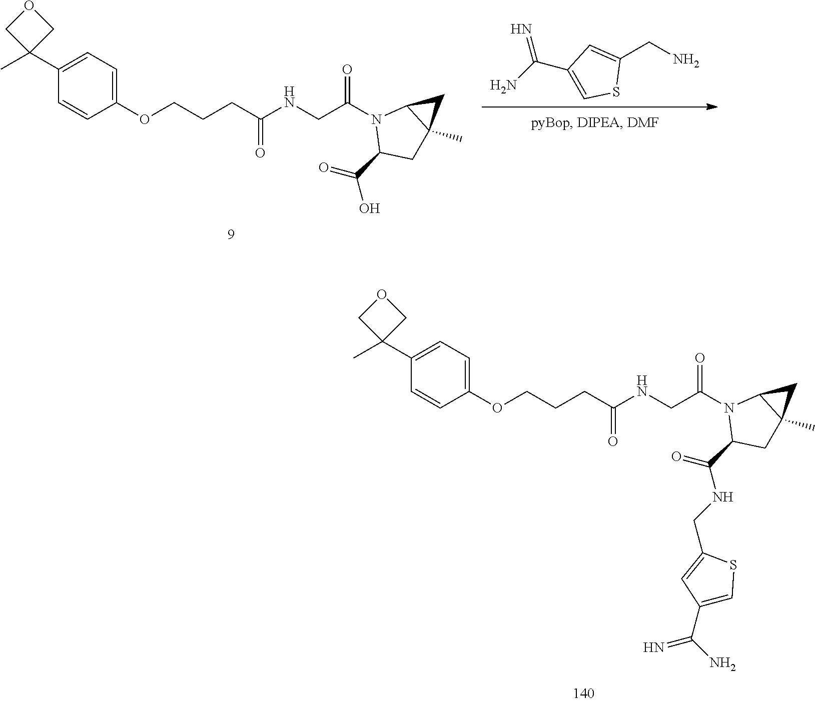

### Step 1: 4,4,5,5-tetramethyl-2-(4-(3-methyloxetan-3-yl)phenyl)-1,3,2-dioxaborolane (2)

To a mixture of 3-(4-bromophenyl)-3-methyloxetane (250 mg, 1.10 mmol) and 4,4,4′,4′,5,5,5′,5′-octamethyl-2,2′-bi(1,3,2-dioxaborolane) (419 mg, 1.65 mmol) in 1,4-dioxane (3 mL) was added KOAc (324 mg, 3.3 mmol), Pd(dppf)Cl₂ (161 mg, 0.22 mmol. The mixture was stirred under N₂ atmosphere at 80° C. for 2 hours before 20 mL of water was added and the mixture was extracted with EA (20 ml×2). The combined organic layers washed with brine, dried over anhydrous Na₂SO₄ and concentrated to dryness under reduced pressure. The residue was purified by flash chromatography (petroleum ether:ethyl acetate=50:1 to 10:1) to give 4,4,5,5-tetramethyl-2-(4-(3-methyloxetan-3-yl)phenyl)-1,3,2-dioxaborolane (300 mg, 99.5% yield) as white solid. LC/MS(ESI) m/z: 275 (M+H)⁺.

### Step 2: 4-(3-methyloxetan-3-yl)phenol (3)

To a solution of 4,4,5,5-tetramethyl-2-(4-(3-methyloxetan-3-yl)phenyl)-1,3,2-dioxaborolane (300 mg, 1.09 mmol) in acetonitrile (3 mL) was added urea hydrogen peroxide (618 mg, 6.57 mmol), 10% citric acid (0.6 mL). The mixture was stirred under N₂ atmosphere at room temperature for 1 hour before 20 mL of water was added and the mixture was extracted with EA (20 ml×2). The combined organic layers washed with brine, dried over anhydrous Na₂SO₄, filtered and concentrated to dryness under reduced pressure. The residue was purified by flash chromatography (petroleum ether:ethyl acetate=50:1 to 10:1) to give 4-(3-methyloxetan-3-yl)phenol (169 mg, 94.4% yield) as yellow oil. LC/MS(ESI) m/z: 165 (M+H)⁺.

### Step 3: ethyl 4-(4-(3-methyloxetan-3-yl)phenoxy)butanoate (4)

To a mixture of 4-(3-methyloxetan-3-yl)phenol (169 mg, 1.02 mmol) in NMP (2 mL) was added ethyl 4-bromobutanoate (1.2 g, 6.12 mmol) and K₂CO₃ (853 mg, 6.12 mmol). The mixture was stirred under N₂ atmosphere at 120° C. for 16 hours before 20 mL of water was added and the mixture was extracted with EA (20 ml×2). The combined organic layers washed with brine, dried over anhydrous Na₂SO₄, filtered and concentrated to dryness under reduced pressure. The residue was purified by flash chromatography (petroleum ether:ethyl acetate=10:1 to 3:1) to give ethyl 4-(4-(3-methyloxetan-3-yl)phenoxy)butanoate (261 mg, 91.9% yield) as yellow oil. LC/MS(ESI) m/z: 279 (M+H)⁺.

### Step 4: 4-(4-(3-methyloxetan-3-yl)phenoxy)butanoic acid (5)

To a solution of 4-(4-(3-methyloxetan-3-yl)phenoxy)butanoate (260 mg, 0.93 mmol) in MeOH (3 mL) and water (1 mL) was added LiOH (118 mg, 2.81 mmol) at 0° C. and the mixture was stirred at room temperature for 16 hours. The mixture was acidified with 1N aq.HCl solution to pH=3 and concentrated to dryness under reduced pressure to give 4-(4-(3-methyloxetan-3-yl)phenoxy)butanoic acid (133 mg, 57.1% yield) as yellow solid. LC/MS (ESI) (m/z): 251(M+H)⁺.

### Step 5: benzyl (4-(4-(3-methyloxetan-3-yl)phenoxy)butanoyl)glycinate (6)

To a mixture of 4-(4-(3-methyloxetan-3-yl)phenoxy)butanoic acid (133 mg, 0.53 mmol) and benzyl 2-aminoacetate (118 mg, 0.58 mmol) in DMF (2 mL) was added DIPEA (411 mg, 3.18 mmol) and T₃P (1.01 g, 1.59 mmol, 50% in ethyl acetate) at 0° C. and the mixture was stirred under N₂ atmosphere at room temperature overnight. The mixture was diluted with ethyl acetate and washed with saturated 1N aq.NaHCO₃ solution. The aqueous layer was extracted with ethyl acetate twice and the combined organic layers were washed with brine, dried over anhydrous Na₂SO₄, filtered and concentrated to dryness under reduced pressure. The residue was purified by flash chromatography on silica gel (petroleum ether:ethyl acetate=1:1) to give benzyl 2-(5-phenoxypentanamido)acetate (165 mg, 78.3% yield) as white solid. LC/MS (ESI) (m/z): 398(M+H)⁺.

### Step 6: (4-(4-(3-methyloxetan-3-yl)phenoxy)butanoyl)glycine (7)

To a solution of benzyl 2-(5-phenoxypentanamido)acetate (165 mg, 0.42 mmol) in MeOH (2 mL) was added Pd/C (20 mg) at room temperature under N₂ atmosphere and the reaction was stirred under a H₂ balloon at room temperature for 3 hours. The mixture was filtered and the filtrate was concentrated to dryness under reduced pressure to give (5-phenoxypentanamido)acetic acid (115 mg, 89.1% yield) as white solid, which was used directly in the next step without further purification. LC/MS (ESI) (m/z):308(M+H)⁺.

### Step 7: ethyl (1S,3S,5S)-5-methyl-2-((4-(4-(3-methyloxetan-3-yl)phenoxy)butanoyl) glycyl)-2-azabicyclo[3.1.0]hexane-3-carboxylate (8)

To a mixture of (5-phenoxypentanamido)acetic acid (115 mg, 0.37 mmol) and methyl pyrrolidine-2-carboxylate (76 mg, 0.45 mmol) in DMF (2 mL) was added DIPEA (287 mg, 2.22 mmol) and T₃P (706 mg, 1.11 mmol, 50% in ethyl acetate) at 0° C. and the mixture was stirred under N₂ atmosphere at room temperature overnight. The mixture was diluted with ethyl acetate and washed with saturated aq.NaHCO₃ solution. The aqueous layer was extracted with ethyl acetate twice and the combined organic layers were washed with brine, dried over anhydrous Na₂SO₄, filtered and concentrated to dryness under reduced pressure. The residue was purified by flash chromatography on silica gel (petroleum ether:Ethyl acetate=1:1) to give ethyl (1S,3S,5S)-5-methyl-2-((4-(4-(3-methyloxetan-3-yl)phenoxy)butanoyl)glycyl)-2-azabicyclo[3.1.0]hexane-3-carboxylate (90 mg, 52.9% yield) as white solid which was used directly in the next step without further purification. LC/MS (ESI) (m/z): 459(M+H)⁺.

### Step 8: (1S,3S,5S)-5-methyl-2-((4-(4-(3-methyloxetan-3-yl)phenoxy)butanoyl)glycyl)-2-azabicyclo[3.1.0]hexane-3-carboxylic acid (9)

To a solution of ethyl (1 S,3S,5S)-5-methyl-2-((4-(4-(3-methyloxetan-3-yl)phenoxy)butanoyl)glycyl)-2-azabicyclo[3.1.0]hexane-3-carboxylate (90 mg, 0.2 mmol) in MeOH (3 mL) and water (1 mL) was added LiOH (25.2 mg, 0.6 mmol) at 0° C. and the mixture was stirred at room temperature for 16 hours. The mixture was acidified with 1N aq.HCl solution to pH<3 and concentrated to dryness under reduced pressure to give (1 S,3S,5S)-5-methyl-2-((4-(4-(3-methyloxetan-3-yl)phenoxy)butanoyl)glycyl)-2-azabicyclo[3.1.0]hexane-3-carboxylic acid (73 mg, 84.9% yield) as white solid, which was used directly in the next step without further purification. LC/MS (ESI) (m/z): 431(M+H)⁺.

### Step 9: (1S,3S,5S)-N-((4-carbamimidoylthiophen-2-yl)methyl)-5-methyl-2-((4-(4-(3-methyloxetan-3-yl)phenoxy)butanoyl)glycyl)-2-azabicyclo[3.1.0]hexane-3-carboxamide (Compound 140)

To a mixture of (1 S,3S,5S)-5-methyl-2-((4-(4-(3-methyloxetan-3-yl)phenoxy)butanoyl)glycyl)-2-azabicyclo[3.1.0]hexane-3-carboxylic acid (39 mg, 0.09 mmol) and 5-(aminomethyl)thiophene-3-carboximidamide (20 mg, 0.13 mmol) in DMF (1 mL) was added DIPEA (68 mg, 0.53 mmol) and (benzotriazol-1-yloxy)tripyrrolidinophosphonium hexafluorophosphate (141 mg, 0.27 mmol) at 0° C. The mixture was stirred under N₂ atmosphere at room temperature overnight. The mixture was diluted with ethyl acetate and washed with saturated aq.NaHCO₃ solution. The aqueous layer was extracted with ethyl acetate twice and the combined organic layers were washed with brine, dried over anhydrous Na₂SO₄, filtered and concentrated to dryness under reduced pressure. The residue was purified by prep-HPLC to give (1 S,3S,5S)-N-((4-carbamimidoylthiophen-2-yl)methyl)-5-methyl-2-((4-(4-(3-methyloxetan-3-yl)phenoxy) butanoyl)glycyl)-2-azabicyclo[3.1.0]hexane-3-carboxamide (3.9 mg, 7.6% yield) as white solid. ¹H NMR (400 MHz, CD₃OD) δ 8.22 (t, J=3.2 Hz, 1H), 7.43 (d, J=15.2 Hz, 1H), 7.16-7.11 (m, 2H), 6.93-6.88 (m, 2H), 4.79 (s, 1H), 4.62 (s, 4H), 4.51 (d, J=9.9 Hz, 2H), 4.17 (q, J=16.7 Hz, 2H), 4.00 (t, J=6.3 Hz, 2H), 2.47 (t, J=7.4 Hz, 2H), 2.39 (t, J=11.8 Hz, 1H), 2.15 (dd, J=13.4, 3.3 Hz, 1H), 2.12-1.97 (m, 3H), 1.67 (s, 3H), 1.28 (s, 3H), 1.12 (dd, J=5.7, 2.5 Hz, 1H), 0.80 (t, J=5.9 Hz, 1H). LC/MS (ESI) (m/z): 568(M+H)⁺.

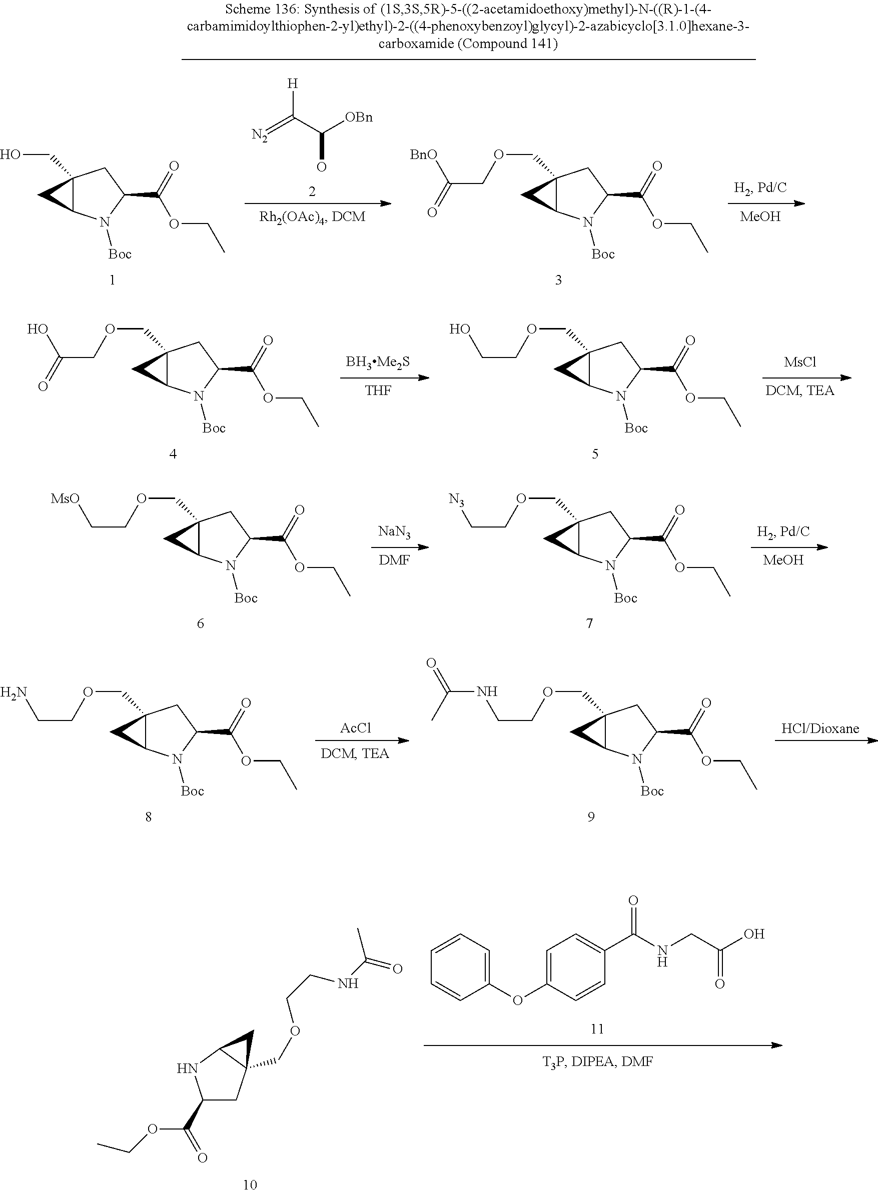

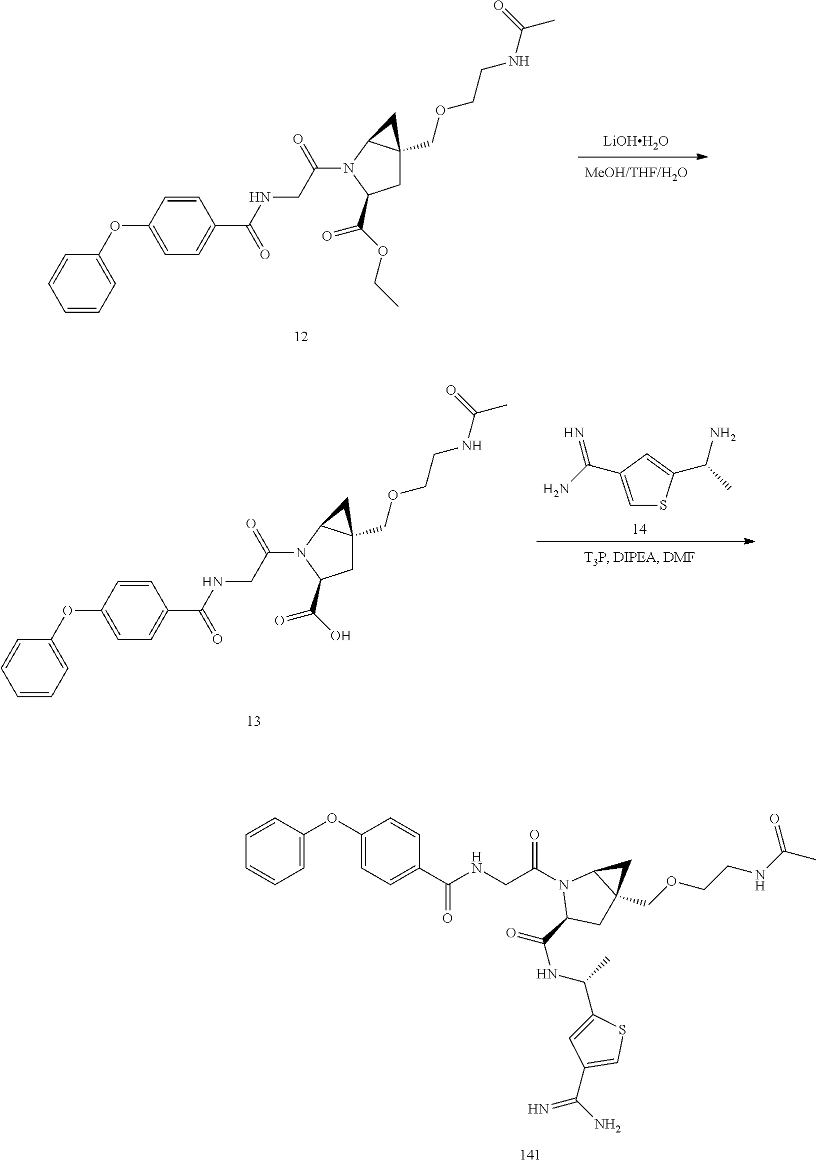

### Step 1: 2-(tert-butyl) 3-ethyl (1S,3S,5R)-5-((2-(benzyloxy)-2-oxoethoxy)methyl)-2-azabicyclo[3.1.0]hexane-2,3-dicarboxylate (3)

To a mixture of 2-(tert-butyl) 3-ethyl (1 S,3S,5R)-5-(hydroxymethyl)-2-azabicyclo[3.1.0]hexane-2,3-dicarboxylate (3.0 g, 10.53 mmol) and Rh₂(OAC)₄ (465 mg, 1.05 mmol) in DCM (30 mL) was added benzyl 2-diazoacetate (6.52 g, 36.9 mmol) in DCM (10 mL) drop-wisely at 0° C. under N₂ atmosphere and the mixture was stirred at 25° C. for 16 hours. The mixture was diluted with H₂O and extracted with ethyl acetate twice. The combined organic layers were washed with brine, dried over anhydrous Na₂SO₄, filtered and concentrated to dryness under reduced pressure. The residue was purified by column chromatography on silica gel (petroleum ether:ethyl acetate=10:1) to give 2-(tert-butyl) 3-ethyl (1 S,3S,5R)-5-((2-(benzyloxy)-2-oxoethoxy)methyl)-2-azabicyclo[3.1.0]hexane-2,3-dicarboxylate (2.78 g, yield 61.0%) as colorless oil. LC/MS (ESI) m/z: 434 (M+H)⁺.

### Step 2: 2-(((1S,3S,5R)-2-(tert-butoxycarbonyl)-3-(ethoxycarbonyl)-2-azabicyclo[3.1.0]hexan-5-yl)methoxy)acetic acid (4)

To a solution of 2-(tert-butyl) 3-ethyl (1S,3S,5R)-5-((2-(benzyloxy)-2-oxoethoxy)methyl)-2-azabicyclo[3.1.0]hexane-2,3-dicarboxylate (2.78 g, 6.42 mmol) in MeOH (35 mL) was added Pd/C (400 mg, 10% wt). The mixture was degassed under N₂ atmosphere for three times and stirred under a H₂ balloon at room temperature for 3 hours. The mixture was filtered and the filtrate was concentrated to dryness under reduced pressure to give 2-(((1 S,3S,5R)-2-(tert-butoxycarbonyl)-3-(ethoxycarbonyl)-2-azabicyclo[3.1.0]hexan-5-yl)methoxy)acetic acid (2.1 g, yield 95.5%) as colorless oil, which was used directly in the next step without further purification. LC/MS (ESI) (m/z): 344 (M+H)⁺.

### Step 3: 2-(tert-butyl) 3-ethyl (1S,3S,5R)-5-((2-hydroxyethoxy)methyl)-2-azabicyclo[3.1.0]hexane-2,3-dicarboxylate (5)

To a solution of 2-(((1 S,3S,5R)-2-(tert-butoxycarbonyl)-3-(ethoxycarbonyl)-2-azabicyclo[3.1.0]hexan-5-yl)methoxy)acetic acid (2.1 g, 6.12 mmol) in THE (30 mL) was added BH₃-Me₂S (9.2 mL, 18.4 mmol, 2M in THF) drop-wisely at 0° C. and the mixture was stirred at 25° C. for 1 hour. The reaction mixture was quenched with MeOH (10 mL) at 0° C. and concentrated to dryness under reduced pressure. The residue was purified by chromatography on silica gel (PE:ethyl acetate=1:1) to give 2-(tert-butyl) 3-ethyl (1 S,3S,5R)-5-((2-hydroxyethoxy)methyl)-2-azabicyclo[3.1.0]hexane-2,3-dicarboxylate (1.67 g, yield 83.5%) as colorless oil. LC/MS (ESI) m/z: 330 (M+H)⁺.

### Step 4: 2-(tert-butyl) 3-ethyl (1S,3S,5R)-5-((2-((methylsulfonyl)oxy)ethoxy)methyl)-2-azabicyclo[3.1.0]hexane-2,3-dicarboxylate (6)

To a solution of 2-(tert-butyl) 3-ethyl (1 S,3S,5R)-5-((2-hydroxyethoxy)methyl)-2-azabicyclo[3.1.0]hexane-2,3-dicarboxylate (1.67 g, 5.08 mmol) in DCM (30 mL) was added TEA (7 mL, 50.8 mmol) followed by drop-wise addition of MsCl (2.0 mL, 25.4 mmol) at 0° C. under N₂ atmosphere and the mixture was stirred at 0° C. for 1 hour. The mixture was diluted with DCM, washed with water and brine, dried over anhydrous Na₂SO₄, filtered and concentrated to dryness under reduced pressure to give 2-(tert-butyl) 3-ethyl (1 S,3S,5R)-5-((2-((methylsulfonyl)oxy)ethoxy)methyl)-2-azabicyclo[3.1.0]hexane-2,3-dicarboxylate (2.0 g, yield 97.1%) as colorless oil, which was used directly in next step without further purification. LC/MS (ESI) m/z: 408 (M+H)⁺.

### Step 5: 2-(tert-butyl) 3-ethyl (1S,3S,5R)-5-((2-azidoethoxy)methyl)-2-azabicyclo[3.1.0]hexane-2,3-dicarboxylate (7)

To a solution of 2-(tert-butyl) 3-ethyl (1S,3S,5R)-5-((2-((methylsulfonyl)oxy)ethoxy)methyl)-2-azabicyclo[3.1.0]hexane-2,3-dicarboxylate (2.0 g, 4.91 mmol) in DMF (30.0 mL) was added NaN₃ (800 mg, 12.28 mmol) and the mixture was stirred at 40° C. for 16 hours. The mixture was diluted with DCM and washed with brine, dried over anhydrous Na₂SO₄, filtered and concentrated to dryness under reduced pressure. The residue was purified by chromatography on silica gel (petroleum ether:ethyl acetate=3:1) to give 2-(tert-butyl) 3-ethyl (1S,3S,5R)-5-((2-azidoethoxy)methyl)-2-azabicyclo[3.1.0]hexane-2,3-dicarboxylate (1.6 g, yield 91.9%) as colorless oil. LC/MS (ESI) m/z: 355 (M+H)⁺.

### Step 6: 2-(tert-butyl) 3-ethyl (1S,3S,5R)-5-((2-aminoethoxy)methyl)-2-azabicyclo[3.1.0]hexane-2,3-dicarboxylate (8)

To a solution of 2-(tert-butyl) 3-ethyl (1S,3S,5R)-5-((2-azidoethoxy)methyl)-2-azabicyclo[3.1.0]hexane-2,3-dicarboxylate (380 mg, 1.07 mmol) in MeOH (7.0 mL) was added Pd/C (40 mg, 10% wt). The mixture was degassed under N₂ atmosphere for three times and stirred under a H₂ balloon at room temperature for 3 hours. The mixture was filtered and filtrate was concentrated to dryness under reduced pressure to give 2-(tert-butyl) 3-ethyl (1 S,3S,5R)-5-((2-aminoethoxy)methyl)-2-azabicyclo[3.1.0]hexane-2,3-dicarboxylate (300.0 mg, yield 85.2%) as colorless oil, which was used directly in next step without further purification. LC/MS (ESI) (m/z): 329 (M+H)⁺.

### Step 7: 2-(tert-butyl) 3-ethyl (1S,3S,5R)-5-((2-acetamidoethoxy)methyl)-2-azabicyclo[3.1.0]hexane-2,3-dicarboxylate (9)

To a solution of 2-(tert-butyl) 3-ethyl (1S,3S,5R)-5-((2-aminoethoxy)methyl)-2-azabicyclo[3.1.0]hexane-2,3-dicarboxylate (300 mg, 0.91 mmol) in DCM (5 mL) was added TEA (0.76 mL, 5.49 mmol) followed by drop-wise addition of AcCl (0.2 mL, 2.73 mmol) at 0° C. under N₂ atmosphere and the mixture was stirred at 0° C. for 1 hour. The mixture was diluted with DCM, washed with brine, dried over anhydrous Na₂SO₄, filtered and concentrated to dryness under reduced pressure. The residue was purified by chromatography on silica gel (DCM:MeOH=95:5) to give 2-(tert-butyl) 3-ethyl (1S,3S,5R)-5-((2-acetamidoethoxy)methyl)-2-azabicyclo[3.1.0]hexane-2,3-dicarboxylate (247 mg, yield 73.1%) as light yellow oil. LC/MS (ESI) m/z: 371 (M+H)⁺.

### Step 8: ethyl (1S,3S,5R)-5-((2-acetamidoethoxy)methyl)-2-azabicyclo[3.1.0]hexane-3-carboxylate (10)

A solution of 2-(tert-butyl) 3-ethyl (1 S,3S,5R)-5-((2-acetamidoethoxy)methyl)-2-azabicyclo[3.1.0]hexane-2,3-dicarboxylate (247 mg, 0.67 mmol) in HCl/1,4-dioxane (5 mL, 4M) was stirred at room temperature for 1 hour. The reaction mixture was concentrated to dryness under reduced pressure, co-evaporated with DCM and dried under vacuum to give ethyl (1 S,3S,5R)-5-((2-acetamidoethoxy)methyl)-2-azabicyclo[3.1.0]hexane-3-carboxylate (180 mg, yield 99.9%) as yellow oil, which was used directly in the next step without further purification. LC/MS (ESI) m/z: 271 (M+H)⁺.

### Step 9: ethyl (1S,3S,5R)-5-((2-acetamidoethoxy)methyl)-2-((4-phenoxybenzoyl)glycyl)-2-azabicyclo[3.1.0]hexane-3-carboxylate (12)

To a mixture of ethyl (1S,3S,5R)-5-((2-acetamidoethoxy)methyl)-2-azabicyclo[3.1.0]hexane-3-carboxylate (90 mg, 0.33 mmol) and (4-phenoxybenzoyl)glycine (99 mg, 0.36 mmol) in DMF (5.0 mL) was added DIPEA (0.33 mL, 1.98 mmol) and T₃P (636 mg, 0.99 mmol, 50% wt in ethyl acetate) at 0° C. under N₂ atmosphere and the mixture was stirred at 25° C. for 16 hours. The mixture was diluted with water and extracted with ethyl acetate twice. The combined organic layers were dried over anhydrous Na₂SO₄, filtered and concentrated to dryness under reduced pressure. The residue was purified by column chromatography on silica gel (DCM:MeOH=95:5) to give ethyl (1S,3S,5R)-5-((2-acetamidoethoxy)methyl)-2-((4-phenoxybenzoyl)glycyl)-2-azabicyclo[3.1.0]hexane-3-carboxylate (100 mg, yield 57.5%) as yellow solid. LC/MS (ESI) m/z: 524 (M+H)⁺.

### Step 10: (1S,3S,5R)-5-((2-acetamidoethoxy)methyl)-2-((4-phenoxybenzoyl)glycyl)-2-azabicyclo[3.1.0]hexane-3-carboxylic acid (13)

To a solution of ethyl (1 S,3S,5R)-5-((2-acetamidoethoxy)methyl)-2-((4-phenoxybenzoyl)glycyl)-2-azabicyclo[3.1.0]hexane-3-carboxylate (100 mg, 0.19 mmol) in MeOH (2 mL) and THF (1 mL) water (1 mL) was added lithium hydroxide (11 mg, 0.27 mmol) at 0° C. and the mixture was stirred at room temperature for 2 hours. The mixture was acidified with 1N aq.HCl to pH-3, extracted with CHCl₃/i-PrOH(V/V=3/1) five times. The combined organic layers were washed with brine, dried over anhydrous Na₂SO₄, filtered and concentrated to dryness under reduced pressure to give (1 S,3S,5R)-5-((2-acetamidoethoxy)methyl)-2-((4-phenoxybenzoyl)glycyl)-2-azabicyclo[3.1.0]hexane-3-carboxylic acid (85 mg, yield 90.4%) as light yellow solid, which was used directly in the next step without further purification. LC/MS (ESI) (m/z): 496 (M+H)⁺.

### Step 11: (1S,3S,5R)-5-((2-acetamidoethoxy)methyl)-N-((R)-1-(4-carbamimidoylthiophen-2-yl)ethyl)-2-((4-phenoxybenzoyl)glycyl)-2-azabicyclo[3.1.0]hexane-3-carboxamide (Compound 141)

To a mixture of (1 S,3S,5R)-5-((2-acetamidoethoxy)methyl)-2-((4-phenoxybenzoyl)glycyl)-2-azabicyclo[3.1.0]hexane-3-carboxylic acid (80 mg, 0.16 mmol) and (R)-5-(1-aminoethyl)thiophene-3-carboximidamide (41 mg, 0.24 mmol) in DMF (3.0 mL) was added DIPEA (0.16 mL, 0.96 mmol) and T₃P (308 mg, 0.48 mmol, 50% wt in ethyl acetate) at 0° C. under N₂ atmosphere and the mixture was stirred at 25° C. for 3 hours. The mixture was diluted with water and extracted with CHCl₃/i-PrOH (V/V=3/1) twice. The combined organic layers were dried over anhydrous Na₂SO₄, filtered and concentrated to dryness under reduced pressure. The residue was purified by column chromatography on silica gel (DCM:MeOH=9:1) and further purified by prep-HPLC to give Compound 141 (5 mg, yield 4.8%) as white solid. ¹H NMR (400 MHz, CD₃OD) δ 8.55 (s, 1H), 8.22 (d, J=1.6 Hz, 1H), 7.87-7.82 (m, 2H), 7.50 (s, 1H), 7.45-7.40 (m, 2H), 7.21 (t, J=7.4 Hz, 1H), 7.08-7.04 (m, 2H), 7.03-6.99 (m, 2H), 5.23 (q, J=6.6 Hz, 1H), 4.41 (d, J=16.4 Hz, 1H), 4.26 (d, J=16.5 Hz, 1H), 3.59 (dd, J=6.8, 3.3 Hz, 2H), 3.53 (t, J=5.6 Hz, 2H), 3.46 (d, J=10.5 Hz, 1H), 3.37 (d, J=4.3 Hz, 2H), 2.69 (dd, J=15.0, 9.7 Hz, 1H), 2.11 (dd, J=13.4, 3.7 Hz, 1H), 1.95 (d, J=2.6 Hz, 3H), 1.91 (s, 1H), 1.58 (d, J=7.0 Hz, 3H), 1.28 (dd, J=5.8, 2.8 Hz, 1H), 0.98 (t, J=5.8 Hz, 1H). LC/MS (ESI) m/z: 647 (M+H)⁺.

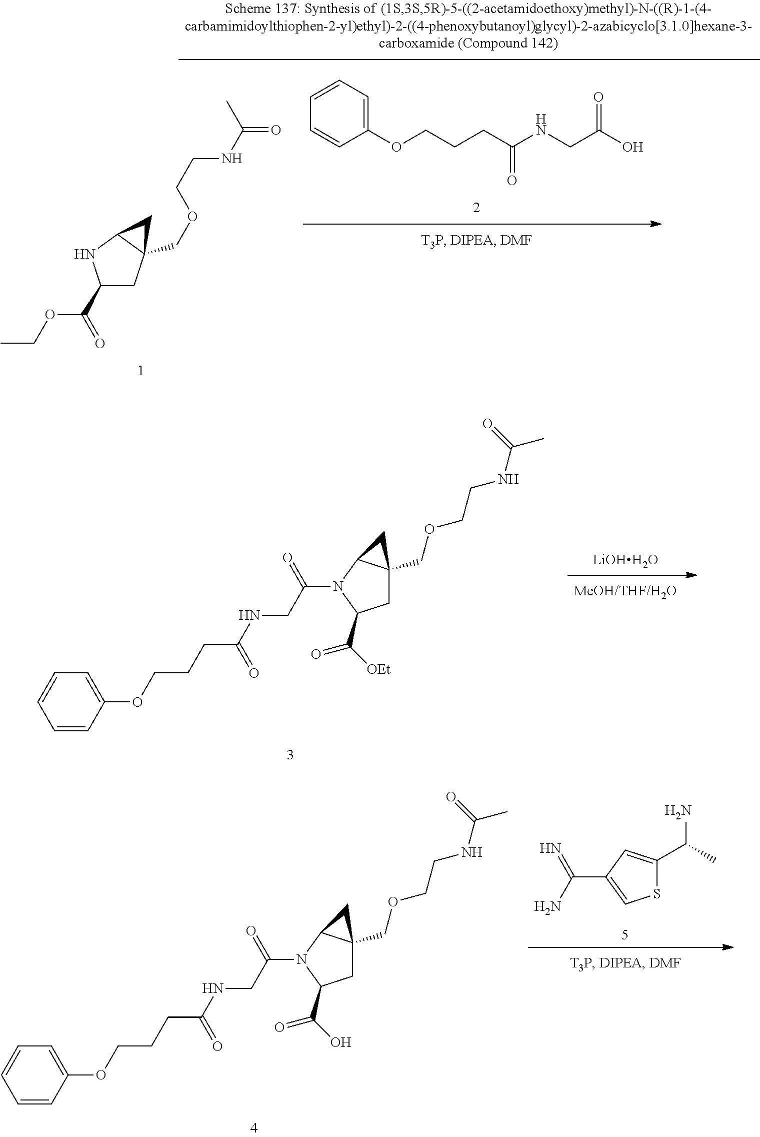

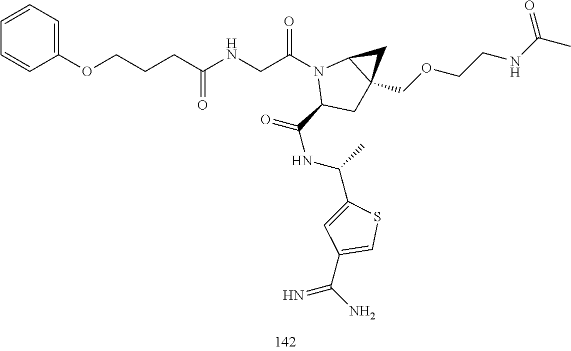 [SMILES: CC(=O)NCCOC[C@@]12C[C@@H]1N(C(=O)CNC(=O)CCCOc1ccccc1)[C@H](C(=O)N[C@H](C)c1cc(C(=N)N)cs1)C2]

### Step 1: ethyl (1S,3S,5R)-5-((2-acetamidoethoxy)methyl)-2-((4-phenoxybutanoyl)glycyl)-2-azabicyclo[3.1.0]hexane-3-carboxylate (3)

To a mixture of ethyl (1S,3S,5R)-5-((2-acetamidoethoxy)methyl)-2-azabicyclo[3.1.0]hexane-3-carboxylate (90 mg, 0.33 mmol) and (4-phenoxybutanoyl)glycine (79 mg, 0.36 mmol) in DMF (5 mL) was added DIPEA (0.33 mL, 1.98 mmol) and T₃P (636 mg, 0.99 mmol) at 0° C. under N₂ atmosphere and the mixture was stirred at 25° C. for 16 hours. The mixture was diluted with water and extracted with ethyl acetate twice. The combined organic layers were dried over anhydrous Na₂SO₄, filtered and concentrated to dryness under reduced pressure. The residue was purified by column chromatography on silica gel (DCM:MeOH=95:5) to give ethyl (1S,3S,5R)-5-((2-acetamidoethoxy)methyl)-2-((4-phenoxybutanoyl)glycyl)-2-azabicyclo[3.1.0]hexane-3-carboxylate (85.0 mg, yield 52.1%) as colorless oil. LC/MS (ESI) m/z: 490 (M+H)⁺.

### Step 2: (1S,3S,5R)-5-((2-acetamidoethoxy)methyl)-2-((4-phenoxybutanoyl)glycyl)-2-azabicyclo[3.1.0]hexane-3-carboxylic acid (4)

To a solution of ethyl (1 S,3S,5R)-5-((2-acetamidoethoxy)methyl)-2-((4-phenoxybutanoyl) glycyl)-2-azabicyclo[3.1.0]hexane-3-carboxylate (85.0 mg, 0.17 mmol) in MeOH (2 mL), THE (1 mL) and water (1 mL) was added lithium hydroxide (11 mg, 0.27 mmol) at 0° C. and the mixture was stirred at room temperature for 2 hours. The mixture was acidified with 1N aq.HCl to pH-3, extracted with CHCl₃/i-PrOH (V/V=3/1) five times. The combined organic layers were washed with brine, dried over anhydrous Na₂SO₄, filtered and concentrated to dryness under reduced pressure to give (1 S,3S,5R)-5-((2-acetamidoethoxy)methyl)-2-((4-phenoxybutanoyl)glycyl)-2-azabicyclo[3.1.0]hexane-3-carboxylic acid (75 mg, yield 93.8%) as light yellow oil, which was used directly in next step without further purification. LC/MS (ESI) (m/z): 462 (M+H)⁺.

### Step 3: (1S,3S,5R)-5-((2-acetamidoethoxy)methyl)-N-((R)-1-(4-carbamimidoylthiophen-2-yl)ethyl)-2-((4-phenoxybutanoyl)glycyl)-2-azabicyclo[3.1.0]hexane-3-carboxamide (Compound 142)

To a mixture of (1 S,3S,5R)-5-((2-acetamidoethoxy)methyl)-2-((4-phenoxybutanoyl)glycyl)-2-azabicyclo[3.1.0]hexane-3-carboxylic acid (75 mg, 0.16 mmol) and (R)-5-(1-aminoethyl)thiophene-3-carboximidamide (27 mg, 0.24 mmol) in DMF (3.0 mL) was added DIPEA (0.16 mL, 0.96 mmol) and T₃P (310 mg, 0.48 mmol) at 0° C. under N₂ atmosphere and the mixture was stirred at 25° C. for 3 hours. The mixture was diluted with water and extracted with CHCl₃/i-PrOH (V/V=3/1) twice. The combined organic layers were dried over anhydrous Na₂SO₄, filtered and concentrated to dryness under reduced pressure. The residue was purified by column chromatography on silica gel (DCM:MeOH=9:1) and further purified by prep-HPLC to give Compound 142 (8 mg, yield 8.0%) as a white solid. ¹H NMR (400 MHz, CD₃OD) δ 8.22 (d, J=1.6 Hz, 1H), 7.50 (dd, J=3.4, 2.1 Hz, 1H), 7.27-7.20 (m, 2H), 6.93-6.86 (m, 3H), 5.31-5.15 (m, 1H), 4.80 (d, J=3.6 Hz, 1H), 4.25 (d, J=16.6 Hz, 1H), 4.09 (d, J=16.6 Hz, 1H), 4.00 (t, J=6.3 Hz, 2H), 3.58 (d, J=10.4 Hz, 1H), 3.53 (t, J=5.5 Hz, 3H), 3.45 (d, J=10.4 Hz, 1H), 3.37-3.33 (m, 2H), 2.66 (t, J=12.3 Hz, 1H), 2.47 (t, J=7.4 Hz, 2H), 2.15-2.04 (m, 3H), 1.95 (s, 3H), 1.59 (dd, J=20.9, 7.0 Hz, 3H), 1.29 (dd, J=5.7, 2.6 Hz, 1H), 1.00 (t, J=5.7 Hz, 1H). LC/MS (ESI) m/z: 613 (M+H)⁺.

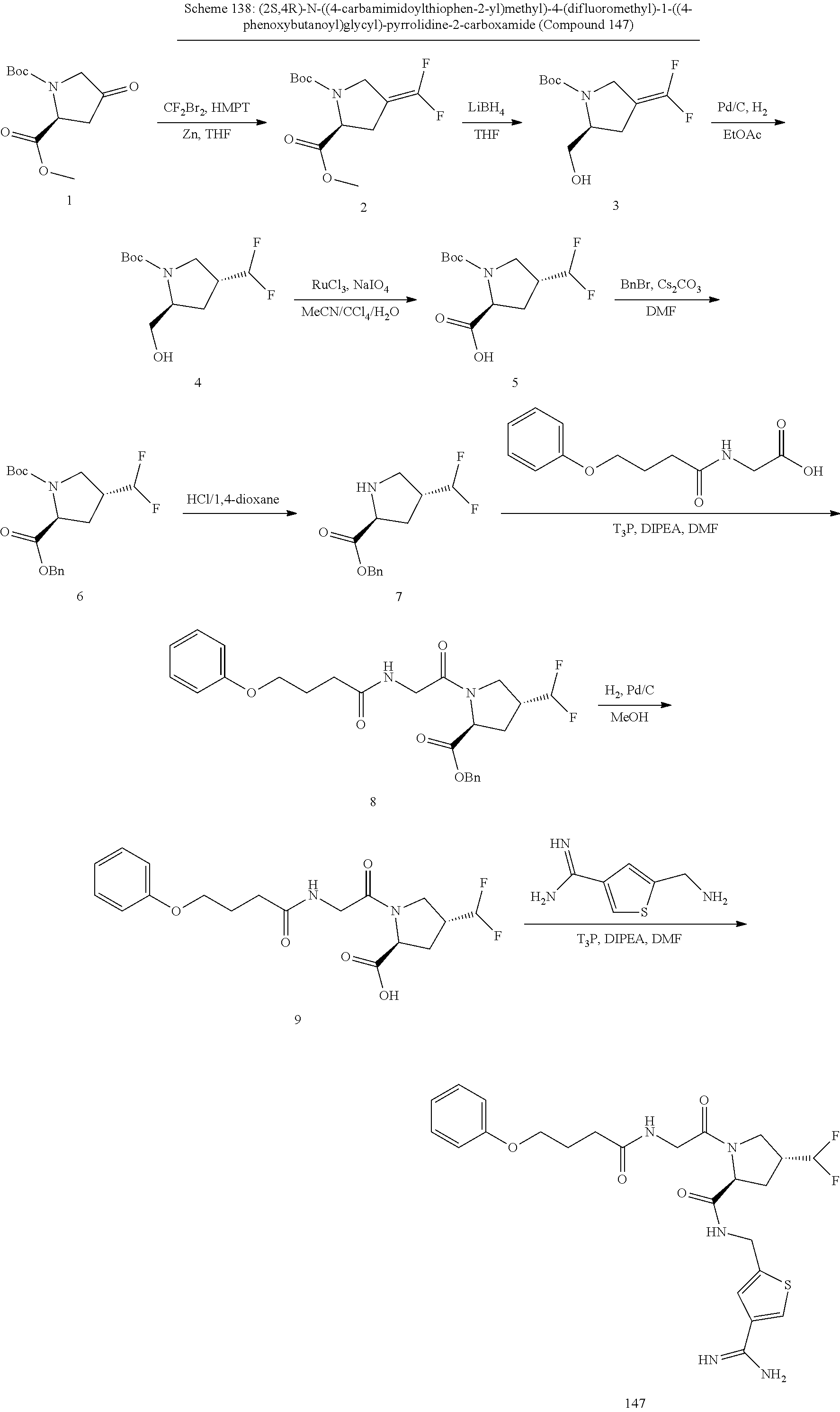

### Step 1: 1-(tert-butyl) 2-methyl (S)-4-(difluoromethylene)pyrrolidine-1,2-dicarboxylate (2)

To a solution of 1-(tert-butyl) 2-methyl (S)-4-oxopyrrolidine-1,2-dicarboxylate (4 g, 16.5 mmol) in THE (40 mL) was added CF₂Br₂ (6 mL, 66.0 mmol) and HMPT (12 mL, 66.0 mmol) at 0° C. The mixture was warmed to room temperature, Zn (4.2 g, 66.0 mmol) and HMPT (1 mL) were added. The reaction was stirred at 65° C. for 4 hours. The reaction mixture was filtered and extracted three times with MTBE. The combined organic layers were washed with brine, dried over anhydrous Na₂SO₄, filtered and concentrated to dryness under reduced pressure. The residue was purified by column chromatography on silica gel (PE:ethyl acetate=10:1) to give 1-(tert-butyl) 2-methyl (S)-4-(difluoromethylene)pyrrolidine-1,2-dicarboxylate (1.7 g, yield 37.2%) as light oil. LC/MS (ESI) m/z: 178 (M−100+H)⁺

### Step 2: tert-butyl (S)-4-(difluoromethylene)-2-(hydroxymethyl)pyrrolidine-1-carboxylate (3)

To a solution of 1-(tert-butyl) 2-methyl (S)-4-(difluoromethylene)pyrrolidine-1,2-dicarboxylate (1.6 g, 5.80 mmol) in THE (10 mL) was added LiBH₄ (5.8 mL, 2M in THF) at 0° C., and the reaction mixture was stirred at 0° C. for 2 hours. The reaction was quenched with saturated aq.NH₄Cl solution and extracted with ethyl acetate three times. The combined organic layers were washed with brine, dried over anhydrous Na₂SO₄, filtered and concentrated to dryness under reduced pressure. The crude residue was purified by column chromatography on silica gel (hexane:ethyl acetate=1:1) to give tert-butyl (S)-4-(difluoromethylene)-2-(hydroxymethyl)pyrrolidine-1-carboxylate (1.1 g, yield 76.5%) as light yellow solid. LC/MS (ESI) m/z: 150 (M−100+H)⁺

### Step 3: tert-butyl (2S,4R)-4-(difluoromethyl)-2-(hydroxymethyl)pyrrolidine-1-carboxylate (4)

To a solution of tert-butyl (S)-4-(difluoromethylene)-2-(hydroxymethyl)pyrrolidine-1-carboxylate (1.1 g, 4.42 mmol) in ethyl acetate (2 mL) was added Pd/C (110 mg, 10% wt) at 0° C., and the reaction mixture was degassed under N₂ atmosphere for three times and stirred under a H₂ balloon at 40° C. for 16 hours. The mixture was filtered and the filtrate was concentrated to dryness under reduced pressure. The residue was purified by silica gel column chromatography (hexane:ethyl acetate=1:1) to give tert-butyl (2S,4R)-4-(difluoromethyl)-2-(hydroxymethyl)pyrrolidine-1-carboxylate (365 mg, yield 33.2%) as light yellow solid. LC/MS (ESI) m/z: 152 (M−100+H)⁺.

### Step 4: (2S,4R)-1-(tert-butoxycarbonyl)-4-(difluoromethyl)pyrrolidine-2-carboxylic acid (5)

To a solution of tert-butyl (2S,4R)-4-(difluoromethyl)-2-(hydroxymethyl)pyrrolidine-1-carboxylate (365 mg, 1.45 mmol) in MeCN (1 mL), CCl₄ (1 mL) and H₂O (1.5 mL) was added Na₁₀₄ (1.2 g, 5.8 mmol) at 25° C. The resulting mixture was stirred for 0.5 hour and RuCl₃ (8 mg, 0.03 mmol) was added and the resulting mixture was stirred at 25° C. for 2 hours. The mixture was filtered off and the filtrate was washed with DCM. The organic layer was separated, and the aqueous phase was extracted with DCM twice. The combined organic layers were dried over anhydrous Na₂SO₄, filtered and concentrated to dryness under reduced pressure. The residue was purified by chromatography on silica gel (DCM:MeOH=10:1) to give (2S,4R)-1-(tert-butoxycarbonyl)-4-(difluoromethyl)pyrrolidine-2-carboxylic acid (263 mg, yield 68.5%) as yellow solid. LC/MS (ESI) m/z: 166 (M−100+H)⁺.

### Step 5: 2-benzyl 1-(tert-butyl) (2S,4R)-4-(difluoromethyl)pyrrolidine-1,2-dicarboxylate(6)

To a solution of (2S,4R)-1-(tert-butoxycarbonyl)-4-(difluoromethyl)pyrrolidine-2-carboxylic acid (263 mg, 0.99 mmol) in DMF (3 mL) was added Cs₂CO₃ (355 mg, 1.09 mmol) followed by BnBr (0.12 mL, 1.19 mmol) at 0° C., and the reaction mixture was stirred at 25° C. for 16 hours. The mixture was diluted with EtOAc, washed with water and brine, dried over anhydrous Na₂SO₄, filtered and concentrated to dryness under reduced pressure. The residue was purified by silica gel column chromatography (hexane:EtOAc=5:1) to give 2-benzyl 1-(tert-butyl) (2S,4R)-4-(difluoromethyl)pyrrolidine-1,2-dicarboxylate (220 mg, yield 62.7%) as light oil. LC/MS (ESI) m/z: 256 (M−100+H)⁺

### Step 6: benzyl (2S,4R)-4-(difluoromethyl)pyrrolidine-2-carboxylate (7)

A solution of 2-benzyl 1-(tert-butyl) (2S,4R)-4-(difluoromethyl)pyrrolidine-1,2-dicarboxylate (220 mg, 0.62 mmol) in HCl/1,4-dioxane (3 mL) was stirred at 0° C. and then warmed to room temperature for 2 hours. The reaction mixture was concentrated to dryness, co-evaporated with DCM and dried under vacuum to give benzyl (2S,4R)-4-(difluoromethyl)pyrrolidine-2-carboxylate (158 mg, yield 99.9%) as yellow solid, which was used directly in the next step without further purification. LC/MS (ESI) m/z: 256 (M+H)⁺

### Step 7: benzyl (2S,4R)-4-(difluoromethyl)-1-((4-phenoxybutanoyl)glycyl)pyrrolidine-2-carboxylate (8)

To a mixture of benzyl (2S,4R)-4-(difluoromethyl)pyrrolidine-2-carboxylate (51 mg, 0.20 mmol) and (4-phenoxybutanoyl)glycine (47 mg, 0.20 mmol) in DMF (2 mL) was added DIPEA (0.2 mL, 1.20 mmol) followed by T₃P (382 mg, 0.60 mmol, 50% wt in EtOAc) at 0° C., and the mixture was stirred at 35° C. for 4 hours. The mixture was diluted with saturated aq.NaHCO₃ solution and extracted with CHCl₃/IPA (3/1, v/v) twice. The combined organic layers were dried over anhydrous Na₂SO₄, filtered and concentrated to dryness under reduced pressure. The residue was purified by chromatography on silica gel (dichloromethane:methanol=20:1) to give benzyl (2S,4R)-4-(difluoromethyl)-1-((4-phenoxybutanoyl)glycyl)pyrrolidine-2-carboxylate (88 mg, yield 93.6%) as yellow oil. LC/MS (ESI) m/z: 475 (M+H)⁺.

### Step 8: (2S,4R)-4-(difluoromethyl)-1-((4-phenoxybutanoyl)glycyl)pyrrolidine-2-carboxylic acid (9)

To a solution of benzyl (2S,4R)-4-(difluoromethyl)-1-((4-phenoxybutanoyl)glycyl)pyrrolidine-2-carboxylate (88 mg, 0.19 mmol) in MeOH (2 mL) was added Pd/C (9 mg, 10% wt) at 0° C. The reaction mixture was degassed under N₂ atmosphere for three times and stirred under a H₂ balloon at room temperature for 2 hours. The mixture was filtered and the filtrate was concentrated to dryness under reduced pressure to give (2S,4R)-4-(difluoromethyl)-1-((4-phenoxybutanoyl)glycyl)pyrrolidine-2-carboxylic acid (66 mg, yield 92.9%) as yellow oil, which was used directly in the next step without further purification. LC/MS (ESI) m/z: 385 (M+H)⁺.

### Step 9: (2S,4R)-N-((4-carbamimidoylthiophen-2-yl)methyl)-4-(difluoromethyl)-1-((4-phenoxybutanoyl)glycyl)pyrrolidine-2-carboxamide (Compound 147)

To a mixture of (2S,4R)-4-(difluoromethyl)-1-((4-phenoxybutanoyl)glycyl)pyrrolidine-2-carboxylic acid (66 mg, 0.17 mmol) and 5-(aminomethyl)thiophene-3-carboximidamide (40 mg, 0.26 mmol) in DMF (2 mL) was added DIPEA (0.2 mL, 1.02 mmol) followed by T₃P (324 mg, 0.51 mmol, 50% wt in EtOAc) at 0° C., and the mixture was stirred at 30° C. for 16 hours. The mixture was quenched with saturated aq.NaHCO₃ solution, extracted with CHCl₃/IPA (3/1, v/v) twice. The combined organic layers were washed with brine, dried over anhydrous Na₂SO₄, filtered and concentrated to dryness under reduced pressure. The residue was purified by prep-HPLC to give Compound 147 (1.1 mg, yield 1.3%) as white solid. ¹H NMR (400 MHz, CD₃OD) δ 8.13 (s, 1H), 7.32 (s, 1H), 7.16-7.12 (m, 2H), 6.82-6.78 (m, 3H), 6.11-5.61 (m, 1H), 5.24 (t, J=4.8 Hz, 1H), 4.46 (s, 3H), 3.96-3.88 (m, 4H), 3.76 (d, J=8.4 Hz, 1H), 3.55 (s, 1H), 2.40-2.34 (m, 3H), 2.16 (d, J=7.6 Hz, 1H), 2.09 (d, J=2.4 Hz, 2H).LC/MS (ESI) m/z: 522 (M+H)⁺.

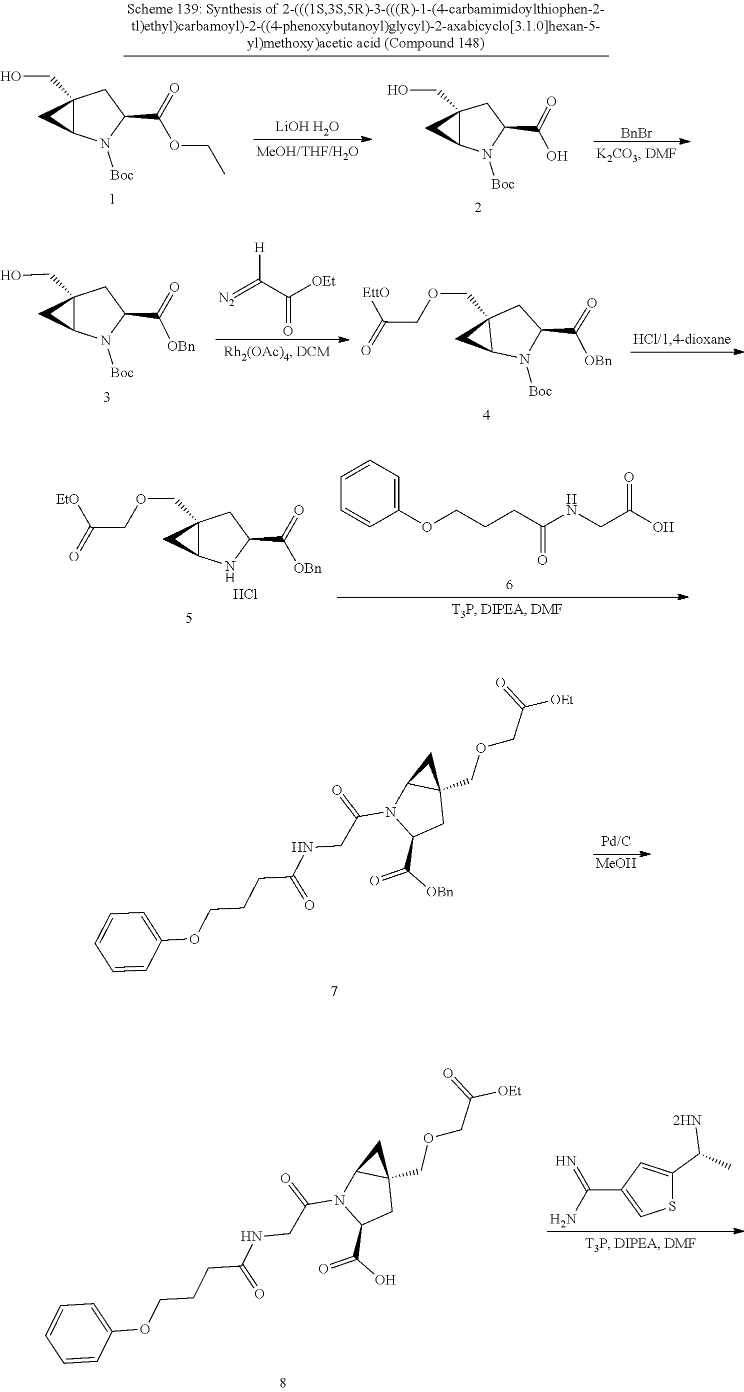

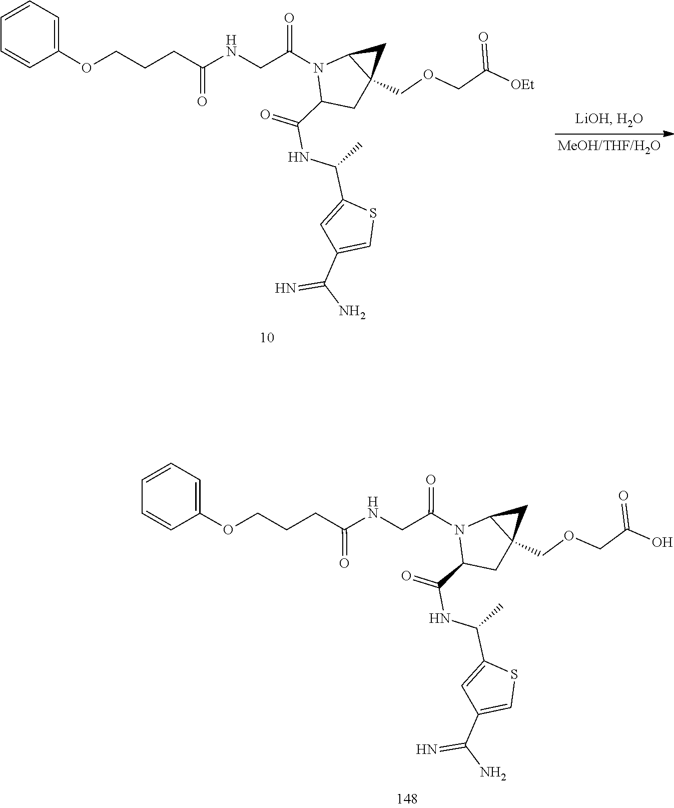 [SMILES: CCOC(=O)COC[C@]12CC(C(=O)N[C@H](C)c3cc(C(=N)N)cs3)N(C(=O)CNC(=O)CCCOc3ccccc3)[C@H]1C2 ; C[C@@H](NC(=O)[C@@H]1C[C@]2(COCC(=O)O)C[C@@H]2N1C(=O)CNC(=O)CCCOc1ccccc1)c1cc(C(=N)N)cs1]

### Step 1: (1S,3S,5R)-2-(tert-butoxycarbonyl)-5-(hydroxymethyl)-2-azabicyclo[3.1.0]hexane-3-carboxylic acid (2)

To a solution of 2-(tert-butyl) 3-ethyl (1S,3S,5R)-5-(hydroxymethyl)-2-azabicyclo[3.1.0]hexane-2,3-dicarboxylate (3.17 g, 11.12 mmol) in MeOH (10 mL), THF (10 mL) and water (4 mL) was added lithium hydroxide (1.4 g, 16.68 mmol) at 0° C. and the mixture was stirred at room temperature for 2 hours. The mixture was acidified with 1N aq.HCl to pH-3, extracted with CHCl₃/i-PrOH (3/1, v/v) five times. The combined organic layers were washed with brine, dried over anhydrous Na₂SO₄, filtered and concentrated to dryness under reduced pressure to give (1 S,3S,5R)-2-(tert-butoxycarbonyl)-5-(hydroxymethyl)-2-azabicyclo[3.1.0]hexane-3-carboxylic acid (2.8 g, yield 98.4%) as light yellow solid, which was used directly in the next step. LC/MS (ESI) (m/z): 258 (M+H)⁺.

### Step 2: 3-benzyl 2-(tert-butyl) (1S,3S,5R)-5-(hydroxymethyl)-2-azabicyclo[3.1.0]hexane-2,3-dicarboxylate (3)

To a solution of (1 S,3S,5R)-2-(tert-butoxycarbonyl)-5-(hydroxymethyl)-2-azabicyclo[3.1.0]hexane-3-carboxylic acid (2.8 g, 10.89 mmol) in DMF (30 mL) was added BnBr (1.55 mL, 13.07 mmol) and K₂CO₃ (3.0 g, 21.78 mol) at 0° C. under N₂ atmosphere and the mixture was stirred at 25° C. for 16 hours. The mixture was diluted with water and extracted with EtOAc twice. The combined organic layers were washed with saturated aq.NH₄Cl solution and brine, dried over anhydrous Na₂SO₄, filtered and concentrated to dryness under reduced pressure. The residue was purified by column chromatography on silica gel (PE:EtOAc=1:1) to give 3-benzyl 2-(tert-butyl) (1S,3S,5R)-5-(hydroxymethyl)-2-azabicyclo[3.1.0]hexane-2,3-dicarboxylate (3.5 g, yield 92.6%) as colorless oil. LC/MS (ESI) m/z: 348 (M+H)⁺.

### Step 3: 3-benzyl 2-(tert-butyl) (1S,3S,5R)-5-((2-ethoxy-2-oxoethoxy)methyl)-2-azabicyclo[3.1.0]hexane-2,3-dicarboxylate (4)

To a mixture of 3-benzyl 2-(tert-butyl) (1S,3S,5R)-5-(hydroxymethyl)-2-azabicyclo[3.1.0]hexane-2,3-dicarboxylate (300 mg, 0.86 mmol) and Rh₂(OAc)₄ (38 mg, 0.086 mmol) in DCM (4 mL) was added ethyl 2-diazoacetate (335 mg, 3.01 mmol) in DCM (1 mL) drop-wisely at 0° C. under N₂ atmosphere and the mixture was stirred at 25° C. for 16 hours. The mixture was diluted with water and extracted with EtOAc twice. The combined organic layers were washed with brine, dried over anhydrous Na₂SO₄, filtered and concentrated to dryness under reduced pressure. The residue was purified by column chromatography on silica gel (PE:EtOAc=5:1) to give 3-benzyl 2-(tert-butyl) (1S,3S,5R)-5-((2-ethoxy-2-oxoethoxy)methyl)-2-azabicyclo[3.1.0]hexane-2,3-dicarboxylate (287 mg, yield 76.7%) as colorless oil. LC/MS (ESI) m/z: 434 (M+H)⁺.

### Step 4: benzyl (1S,3S,5R)-5-((2-ethoxy-2-oxoethoxy)methyl)-2-azabicyclo[3.1.0]hexane-3-carboxylate hydrochloride (5)

A solution of 3-benzyl 2-(tert-butyl) (1 S,3S,5R)-5-((2-ethoxy-2-oxoethoxy)methyl)-2-azabicyclo[3.1.0]hexane-2,3-dicarboxylate (287 mg, 0.66 mmol) in HCl/1,4-dioxane (5 mL, 4M) was stirred at room temperature for 2 hours. The reaction mixture was concentrated to dryness under reduced pressure, co-evaporated with DCM and dried under vacuum to give benzyl (1 S,3S,5R)-5-((2-ethoxy-2-oxoethoxy)methyl)-2-azabicyclo[3.1.0]hexane-3-carboxylate (240 mg, yield 99.9%) as yellow oil, which was used directly in the next step without further purification. LC/MS (ESI) m/z: 334 (M+H)⁺.

### Step 5: benzyl (1S,3S,5R)-5-((2-ethoxy-2-oxoethoxy)methyl)-2-((4-phenoxybutanoyl)glycyl)-2-azabicyclo[3.1.0]hexane-3-carboxylate (7)

To a mixture of benzyl (1 S,3S,5R)-5-((2-ethoxy-2-oxoethoxy)methyl)-2-azabicyclo[3.1.0]hexane-3-carboxylate hydrochloride (240 mg, 0.66 mmol) and (4-phenoxybutanoyl)glycine (156 mg, 0.66 mmol) in DMF (5 mL) was added DIPEA (0.65 mL, 3.96 mmol) and T₃P (1.26 g, 1.98 mmol, 50% wt in EtOAc) at 0° C. under N₂ atmosphere and the mixture was stirred at 25° C. for 16 hours. The mixture was diluted with water and extracted with EtOAc twice. The combined organic layers were dried over anhydrous Na₂SO₄, filtered and concentrated to dryness under reduced pressure. The residue was purified by column chromatography on silica gel (DCM:MeOH=95:5) to give benzyl (1S,3S,5R)-5-((2-ethoxy-2-oxoethoxy)methyl)-2-((4-phenoxybutanoyl)glycyl)-2-azabicyclo[3.1.0]hexane-3-carboxylate (200 mg, yield 54.8%) as colorless solid. LC/MS (ESI) m/z: 553 (M+H)⁺.

### Step 6: (1S,3S,5R)-5-((2-ethoxy-2-oxoethoxy)methyl)-2-((4-phenoxybutanoyl)glycyl)-2-azabicyclo[3.1.0]hexane-3-carboxylic acid (8)

To a solution of benzyl (1 S,3S,5R)-5-((2-ethoxy-2-oxoethoxy)methyl)-2-((4-phenoxybutanoyl)glycyl)-2-azabicyclo[3.1.0]hexane-3-carboxylate (200 mg, 0.36 mmol) in MeOH (5 mL) was added Pd/C (40 mg, 10% wt). The mixture was degassed under N₂ atmosphere for three times and stirred under a H₂ balloon at room temperature for 3 hours. The mixture was filtered and the filtrate was concentrated to dryness under reduced pressure to give (1 S,3S,5R)-5-((2-ethoxy-2-oxoethoxy)methyl)-2-((4-phenoxybutanoyl)glycyl)-2-azabicyclo[3.1.0]hexane-3-carboxylic acid (150 mg, yield 89.8%) as colorless oil, which was used directly in the next step. LC/MS (ESI) (m/z): 463 (M+H)⁺.

### Step 7: ethyl 2-(((1S,3S,5R)-3-(((R)-1-(4-carbamimidoylthiophen-2-yl)ethyl)carbamoyl)-2-((4-phenoxybutanoyl)glycyl)-2-azabicyclo[3.1.0]hexan-5-yl)methoxy)acetate (10)

To a solution of (1 S,3S,5R)-5-((2-ethoxy-2-oxoethoxy)methyl)-2-((4-phenoxybutanoyl)glycyl)-2-azabicyclo[3.1.0]hexane-3-carboxylic acid (150 mg, 0.32 mmol) and (R)-5-(1-aminoethyl)thiophene-3-carboximidamide (82 mg, 0.48 mmol) in DMF (3.0 mL) was added DIPEA (0.3 mL, 1.92 mmol) and T₃P (618 mg, 0.96 mmol, 50% wt in EtOAc) at 0° C. under N₂ atmosphere and the mixture was stirred at 25° C. for 3 hours. The mixture was diluted with water and extracted with CHCl₃/i-PrOH (3/1, v/v) twice. The combined organic layers were dried over anhydrous Na₂SO₄, filtered and concentrated to dryness under reduced pressure. The residue was purified by prep-TLC (DCM:MeOH=9:1) to give ethyl 2-(((1 S,3S,5R)-3-(((R)-1-(4-carbamimidoylthiophen-2-yl)ethyl)carbamoyl)-2-((4-phenoxybutanoyl)glycyl)-2-azabicyclo[3.1.0]hexan-5-yl)methoxy)acetate (36 mg, yield 18.1%) as white solid. LC/MS (ESI) m/z: 614 (M+H)⁺.

### Step 8: 2-(((1S,3S,5R)-3-(((R)-1-(4-carbamimidoylthiophen-2-yl)ethyl)carbamoyl)-2-((4-phenoxybutanoyl)glycyl)-2-azabicyclo[3.1.0]hexan-5-yl)methoxy)acetic acid (Compound 148)

To a solution of ethyl 2-(((1 S,3S,5R)-3-(((R)-1-(4-carbamimidoylthiophen-2-yl)ethyl)carbamoyl)-2-((4-phenoxybutanoyl)glycyl)-2-azabicyclo[3.1.0]hexan-5-yl)methoxy) acetate (36 mg, 0.06 mmol) in MeOH (2 mL), THE (1 mL) and water (1 mL) was added lithium hydroxide (3.6 mg, 0.09 mmol) at 0° C. and the mixture was stirred at room temperature for 2 hours. The mixture was acidified with 1N aq.HCl to pH3 and concentrated to dryness under reduced pressure. The residue was purified by prep-HPLC to give Compound 148 (9.5 mg, yield 27.9%) as a white solid. ¹H NMR (400 MHz, CD₃OD) δ 8.21 (d, J=1.5 Hz, 1H), 7.51 (s, 1H), 7.27-7.20 (m, 2H), 6.92-6.84 (m, 3H), 5.19 (t, J=7.0 Hz, 1H), 4.81 (d, J=3.7 Hz, 1H), 4.18 (q, J=16.7 Hz, 2H), 3.99 (t, J=6.3 Hz, 2H), 3.91 (d, J=2.1 Hz, 2H), 3.67-3.62 (m, 1H), 3.57 (dd, J=6.1, 2.6 Hz, 1H), 3.44 (t, J=14.5 Hz, 1H), 2.75 (t, J=12.5 Hz, 1H), 2.46 (q, J=7.1 Hz, 2H), 2.14-2.02 (m, 3H), 1.59 (dd, J=20.7, 6.9 Hz, 3H), 1.22 (dd, J=5.6, 2.5 Hz, 1H), 1.01 (t, J=5.9 Hz, 1H). LC/MS (ESI) m/z: 586 (M+H)⁺.

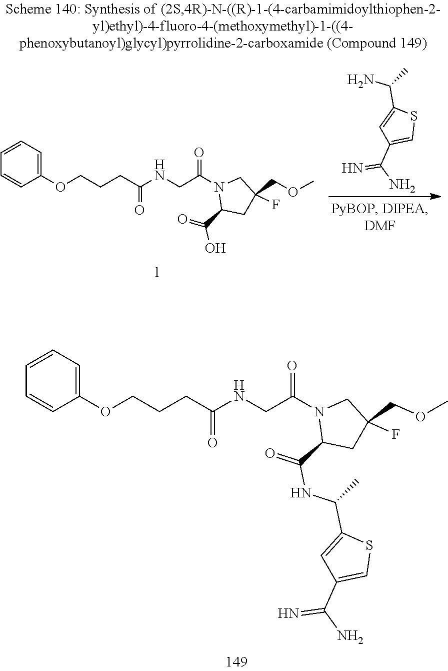 [SMILES: COC[C@@]1(F)C[C@@H](C(=O)O)N(C(=O)CNC(=O)CCCOc2ccccc2)C1 ; C[C@@H](N)c1cc(C(=N)N)cs1 ; COC[C@@]1(F)C[C@@H](C(=O)N[C@H](C)c2cc(C(=N)N)cs2)N(C(=O)CNC(=O)CCCOc2ccccc2)C1]

Step 1: (2S,4R)-N-((R)-1-(4-carbamimidoylthiophen-2-yl) ethyl)-4-fluoro-4-(methoxymethyl)-1-((4-phenoxybutanoyl)glycyl)pyrrolidine-2-carboxamide (Compound 149)

To a solution of (2S,4R)-4-fluoro-4-(methoxymethyl)-1-((4-phenoxybutanoyl) glycyl) pyrrolidine-2-carboxylic acid (40 mg, 0.10 mmol) in DMF (4 mL) was added DIPEA (65 mg, 0.50 mmol) and PyBOP (78 mg, 0.15 mmol) and the mixture was stirred at room temperature for 30 minutes. The mixture was diluted with ethyl acetate and washed with water. The aqueous layer was extracted with CHCl₃/i-PrOH (5×10 mL, v/v=3/1) and the combined organic layers were washed with brine, dried over anhydrous Na₂SO₄, filtered and concentrated to dryness under reduced pressure. The residue was purified by prep-HPLC to give (2S,4R)-N-((R)-1-(4-carbamimidoylthiophen-2-yl) ethyl)-4-fluoro-4-(methoxymethyl)-1-((4-phenoxybutanoyl) glycyl)pyrrolidine-2-carboxamide (3.7 mg, yield 6.8%) as white solid. ¹H NMR (400 MHz, CD₃OD) δ 8.55 (s, 1H, FA), 8.23 (d, J=1.6 Hz, 1H), 7.55 (d, J=12.8 Hz, 1H), 7.26-7.21 (m, 2H), 6.91-6.87 (m, 3H), 5.34-5.21 (m, 1H), 4.58 (t, J=8.6 Hz, 2H), 4.03-3.84 (m, 5H), 3.69-3.62 (m, 2H), 3.41 (s, 3H), 2.56-2.44 (m, 3H), 2.23-2.05 (m, 3H), 1.60 (dd, J=22.8, 22.8 Hz, 3H). LC/MS (ESI) (m/z): 548 (M+H)⁺.

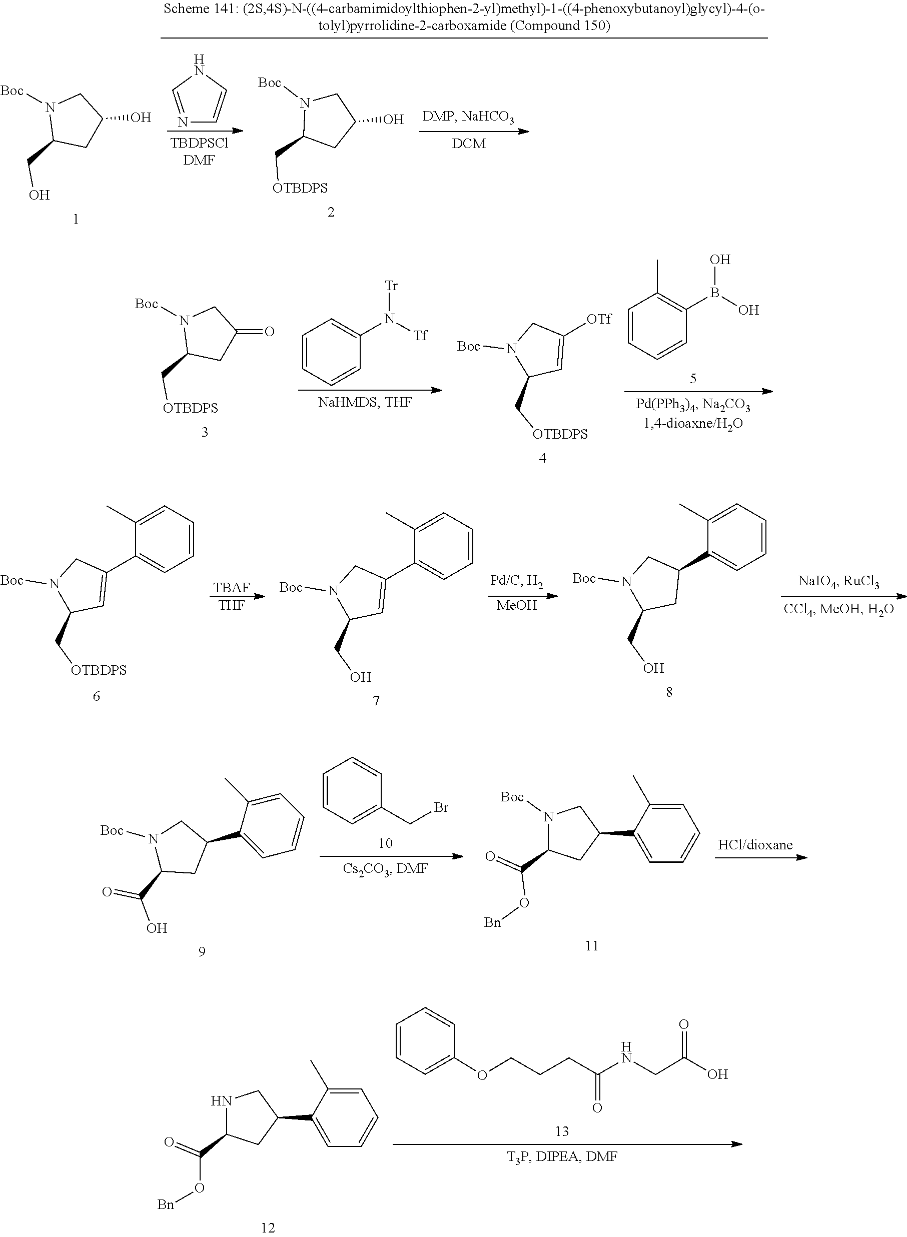

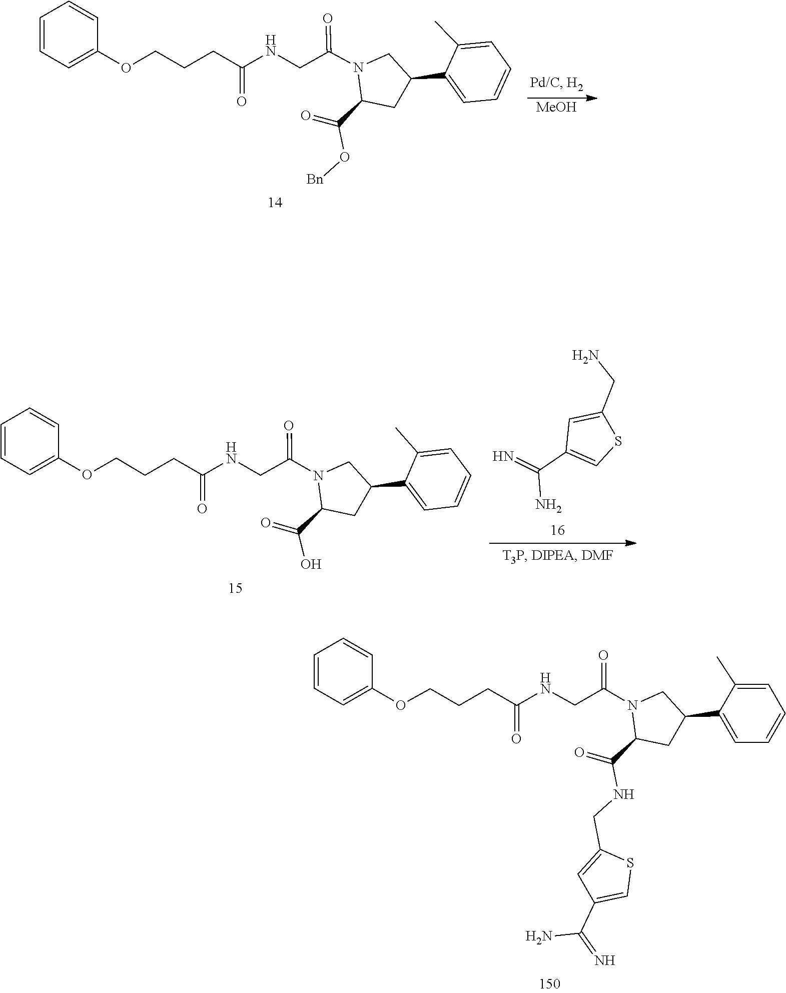

### Step 1: tert-butyl (2S,4R)-2-(((tert-butyldiphenylsilyl)oxy)methyl)-4-hydroxypyrrolidine-1-carboxylate

To a mixture of tert-butyl (2S,4R)-4-hydroxy-2-(hydroxymethyl)pyrrolidine-1-carboxylate (19.0 g, 86 mmol) and 1H-imidazole (11.71 g, 170 mmol) in DMF (200 ml) which was added TBDPSCl (26.0 g, 95 mmol) at 0° C. under N₂ atmosphere and the mixture was stirred for 4 hours. The mixture was diluted with water and extracted with ethyl acetate twice. The combined organic layers were washed with brine, dried over anhydrous Na₂SO₄, filtered and concentrated to dryness under reduced pressure. The residue was purified by column chromatography on silica gel (PE:Ethyl Acetate=5:1) to give tert-butyl (2S,4R)-2-(((tert-butyldiphenylsilyl)oxy)methyl)-4-hydroxypyrrolidine-1-carboxylate (22 g, yield 56%) as white solid. LCMS (ESI) m/z=456 (M+H)⁺.

### Step 2: tert-butyl (S)-2-(((tert-butyldiphenylsilyl)oxy)methyl)-4-oxopyrrolidine-1-carboxylate

To a solution of tert-butyl (2S,4R)-2-(((tert-butyldiphenylsilyl)oxy)methyl)-4-hydroxypyrrolidine-1-carboxylate (20.0 g, 43.9 mmol) in DCM (200 ml) was added DMP (18.6 g, 43.9 mmol) and NaHCO₃ (3.5 g, 43.9 mmol) at 0° C. under N₂ atmosphere and the mixture was stirred overnight at room temperature. The mixture was diluted with water and extracted with ethyl acetate twice. The combined organic layers were washed with brine, dried over anhydrous Na₂SO₄, filtered and concentrated to dryness under reduced pressure. The residue was purified by column chromatography on silica gel (PE:Ethyl Acetate=10:1) to give tert-butyl (S)-2-(((tert-butyldiphenylsilyl)oxy)methyl)-4-oxopyrrolidine-1-carboxylate (9.5 g, yield 47.8%). LC/MS (ESI) (m/z): 454 (M+H)⁺.

### Step 3: tert-butyl (S)-2-(((tert-butyldiphenylsilyl)oxy)methyl)-4-(((trifluoromethyl) sulfonyl)oxy)-2,5-dihydro-1H-pyrrole-1-carboxylate

To a solution of tert-butyl (S)-2-(((tert-butyldiphenylsilyl)oxy)methyl)-4-oxopyrrolidine-1-carboxylate (8 g, 17.6 mmol) in THE was added NaHMDS (606 mg, 3.3 mmol) dropwise under N₂ atmosphere at −78° C. The mixture was stirred for 30 minutes before 1,1,1-trifluoro-N-phenyl-N-((trifluoromethyl) sulfonyl)methanesulfonamide (1.2 g, 3.3 mmol) was added. The mixture was allowed to stir at room temperature overnight before diluted with water and extracted with ethyl acetate twice. The combined organic layers were washed with brine, dried over anhydrous Na₂SO₄, filtered, and concentrated to dryness under reduced pressure. The residue was purified by column chromatography on silica gel (PE:Ethyl Acetate=3:1) to give the title compound tert-butyl (S)-2-(((tert-butyldiphenylsilyl)oxy)methyl)-4-(((trifluoromethyl)sulfonyl) oxy)-2,5-dihydro-1H-pyrrole-1-carboxylate (9.2 g, yield 87.2%) as colorless oil. LCMS (ESI) m/z=586.2 (M+H)⁺.

### Step 4: tert-butyl (S)-2-(((tert-butyldiphenylsilyl)oxy)methyl)-4-(o-tolyl)-2,5-dihydro-1H-pyrrole-1-carboxylate

To a mixture of tert-butyl (S)-2-(((tert-butyldiphenylsilyl)oxy)methyl)-4-(((trifluoromethyl)sulfonyl)oxy)-2,5-dihydro-1H-pyrrole-1-carboxylate (1.0 g, 1.7 mmol)) and Pd(PPh₃)₄ (200 mg, 0.17 mmol) in 1,4-dioxane (10 ml)/water (3 ml) was added o-tolylboronic acid (280 mg, 2.0 mmol) and Na₂CO₃ (540 mg, 5.1 mmol) under N₂ atmosphere at 0° C. The mixture was stirred at 80° C. overnight before diluted with water and extracted with ethyl acetate twice. The combined organic layers were washed with brine, dried over anhydrous Na₂SO₄, filtered and concentrated to dryness under reduced pressure. The residue was purified by flash column chromatography on silica gel (PE:Ethyl Acetate=10:1) to give tert-butyl (S)-2-(((tert-butyldiphenylsilyl)oxy)methyl)-4-(o-tolyl)-2,5-dihydro-1H-pyrrole-1-carboxylate (520 mg, yield 57.6%) as white solid. LCMS (ESI) m/z=528 (M+H)⁺.

### Step 5: tert-butyl (S)-2-(hydroxymethyl)-4-(o-tolyl)-2,5-dihydro-1H-pyrrole-1-carboxylate

To a solution of tert-butyl (S)-2-(((tert-butyldiphenylsilyl)oxy)methyl)-4-(o-tolyl)-2,5-dihydro-1H-pyrrole-1-carboxylate (520 mg, 0.98 mmol) in THE (3 mL) was added TBAF (515 mg, 2.0 mmol) at 0° C. and the mixture was stirred under N₂ atmosphere at room temperature for 16 hours. The mixture was diluted with saturated aq.NH₄Cl solution and extracted with ethyl acetate twice. The combined organic layers were washed with brine, dried over anhydrous Na₂SO₄, filtered and concentrated to dryness under reduced pressure. The residue was purified by column chromatography on silica gel (PE:Ethyl Acetate=10:1) to give tert-butyl (S)-2-(hydroxymethyl)-4-(o-tolyl)-2,5-dihydro-1H-pyrrole-1-carboxylate (230 mg, yield 80.7%) as white solid. LCMS (ESI) m/z=290.2 (M+H)⁺.

### Step 6: tert-butyl (2S,4R)-2-(hydroxymethyl)-4-(o-tolyl)pyrrolidine-1-carboxylate

To a solution of tert-butyl (S)-2-(hydroxymethyl)-4-(o-tolyl)-2,5-dihydro-1H-pyrrole-1-carboxylate (110 mg, 0.38 mmol) in MeOH (1.2 mL) was added Pd/C (100 mg, 10% wt) at room temperature and the mixture was stirred under a H₂ balloon at room temperature for 2 hours. The mixture was filtered and the filtrate was concentrated to dryness under reduced pressure to give tert-butyl (2S,4R)-2-(hydroxymethyl)-4-(o-tolyl)pyrrolidine-1-carboxylate (100 mg, yield 90.3%) as white solid, which was used directly in the next step without further purification. LC/MS (ESI) (m/z): 292(M+H)⁺.

### Step 7: (2S,4R)-1-(tert-butoxycarbonyl)-4-(o-tolyl)pyrrolidine-2-carboxylic acid

To a mixture of tert-butyl (2S,4R)-2-(hydroxymethyl)-4-(o-tolyl)pyrrolidine-1-carboxylate (200 mg, 0.69 mmol) and NaIO₄ (440 mg, 2.06 mmol) in CCl₄ (0.8 ml)/CH₃CN (0.8 ml)/water (1.2 ml) was stirred under N₂ atmosphere at room temperature for 20 minutes. The reaction mixture was added RuCl₃ (8 mg, 0.041 mmol) and stirred for 4 hours. The mixture was diluted with water and extracted with ethyl acetate twice. The combined organic layers were washed with brine, dried over anhydrous Na₂SO₄, filtered and concentrated to dryness under reduced pressure. The residue was purified by column chromatography on silica gel (PE:Ethyl Acetate=3:1) to give (2S,4R)-1-(tert-butoxycarbonyl)-4-(o-tolyl)pyrrolidine-2-carboxylic acid (200 mg, yield 95.2%) as white solid. LCMS (ESI) m/z=250(M+H−56)⁺.

### Step 8: 2-benzyl 1-(tert-butyl) (2S,4R)-4-(o-tolyl)pyrrolidine-1,2-dicarboxylate

To a mixture of (2S,4R)-1-(tert-butoxycarbonyl)-4-(o-tolyl)pyrrolidine-2-carboxylic acid (200 mg, 0.69 mmol) and Cs₂CO₃ (670 mg, 2.06 mmol) in DMF (2.5 ml) was added benzyl bromide (129 mg, 0.76 mmol) under N₂ atmosphere at room temperature and the reaction mixture was stirred overnight. The mixture was diluted with water and extracted with ethyl acetate twice. The combined organic layers were washed with brine, dried over anhydrous Na₂SO₄, filtered and concentrated to dryness under reduced pressure. The residue was purified by column chromatography on silica gel (PE:Ethyl Acetate=3:1) to give benzyl 2-(5-phenoxypentanamido)acetate (160 mg, yield 91.0%) as white solid. LCMS (ESI) m/z=396 (M+H)⁺.

### Step 9: benzyl (2S,4R)-4-(o-tolyl)pyrrolidine-2-carboxylate

To a mixture of 2-benzyl 1-(tert-butyl) (2S,4R)-4-(o-tolyl)pyrrolidine-1,2-dicarboxylate (160 mg, 0.40 mmol) in HCl/1,4-dioxane (2 mL) was stirred under N₂ atmosphere at room temperature for 2 hours.

The reaction mixture was concentrated to dryness under reduced pressure, diluted with DCM again and dried under vacuum to give benzyl (2S,4R)-4-(o-tolyl)pyrrolidine-2-carboxylate (110 mg, yield 92.3%), which was used directly in the next step without further purification. LC/MS (ESI) m/z: 296 (M+H)⁺.

### Step 10: benzyl (2S,4R)-1-((4-phenoxybutanoyl)glycyl)-4-(o-tolyl)pyrrolidine-2-carboxylate

To a mixture of benzyl (2S,4R)-4-(o-tolyl)pyrrolidine-2-carboxylate (110 mg, 0.37 mmol) and (4-phenoxybutanoyl)glycine (132 mg, 0.56 mmol) in DMF (1.3 mL) was added DIPEA (288 mg, 2.24 mmol) and T₃P (356 mg, 1.12 mmol) at 0° C. and the mixture was stirred under N₂ atmosphere at room temperature for 2 hours. The organic layer was diluted with water, extracted with ethyl acetate twice and the combined organic layers were washed with brine, dried over anhydrous Na₂SO₄, filtered and concentrated to dryness under reduced pressure. The residue was purified by flash chromatography on silica gel (petroleum ether:ethyl acetate=3:1) to give benzyl (2S,4R)-1-((4-phenoxybutanoyl)glycyl)-4-(o-tolyl)pyrrolidine-2-carboxylate (77 mg, yield 40%) as white solid. LC/MS (ESI) (m/z): 515(M+H)⁺.

### Step 11: (2S,4R)-1-((4-phenoxybutanoyl)glycyl)-4-(o-tolyl)pyrrolidine-2-carboxylic acid

To a solution of benzyl (2S,4R)-1-((4-phenoxybutanoyl)glycyl)-4-(o-tolyl)pyrrolidine-2-carboxylate (77 mg, 0.15 mmol) in MeOH (1.2 mL) was added Pd/C (70 mg, 10% wt) at room temperature and the mixture was stirred under a H₂ balloon for 2 hours. The mixture was filtered and the filtrate was concentrated to dryness under reduced pressure to give (2S,4R)-1-((4-phenoxybutanoyl)glycyl)-4-(o-tolyl)pyrrolidine-2-carboxylic acid (60 mg, yield 94.5%) as colorless oil, which was used directly in the next step without further purification. LC/MS (ESI) (m/z): 425(M+H)⁺.

### Step 12: (2S,4S)-N-((4-carbamimidoylthiophen-2-yl)methyl)-1-((4-phenoxybutanoyl)glycyl)-4-(o-tolyl)pyrrolidine-2-carboxamide (Compound 150)

To a mixture of (2S,4R)-1-((4-phenoxybutanoyl)glycyl)-4-(o-tolyl)pyrrolidine-2-carboxylic acid (60 mg, 0.14 mmol) and 5-(aminomethyl)thiophene-3-carboximidamide (33 mg, 0.21 mmol) in DMF (1 mL) was added DIPEA (110 mg, 0.85 mmol) and PyBOP (90 mg, 0.17 mmol) at 0° C. and the mixture was stirred under N₂ atmosphere at room temperature for 2 hours. The organic layer was diluted with water extracted with ethyl acetate twice and the combined organic layers were washed with brine, dried over anhydrous Na₂SO₄, filtered and concentrated to dryness under reduced pressure. The residue was purified by flash chromatography on silica gel (petroleum ether:ethyl acetate=3:1) to give Compound 150 (8 mg, yield 10.0%) as a white solid. ¹H NMR (400 MHz, CD₃OD) δ 8.22 (d, J=1.6 Hz, 1H), 7.46 (s, 1H), 7.31 (d, J=7.8 Hz, 1H), 7.26-7.22 (m, 2H), 7.16 (dt, J=6.0, 5.3 Hz, 3H), 6.91-6.87 (m, 3H), 4.51 (d, J=9.6 Hz, 1H), 4.15 (d, J=16.8 Hz, 1H), 4.08 (d, J=7.3 Hz, 1H), 4.02-4.00 (m, 2H), 3.98 (d, J=6.4 Hz, 1H), 3.65-3.57 (m, 2H), 2.47 (t, J=7.5 Hz, 2H), 2.40 (s, 3H), 2.35 (d, J=15.0 Hz, 1H), 2.21-2.17 (m, 1H), 2.12-2.07 (m, 2H), 2.04 (s, 2H). LCMS (ESI) m/z=562(M+H)⁺.

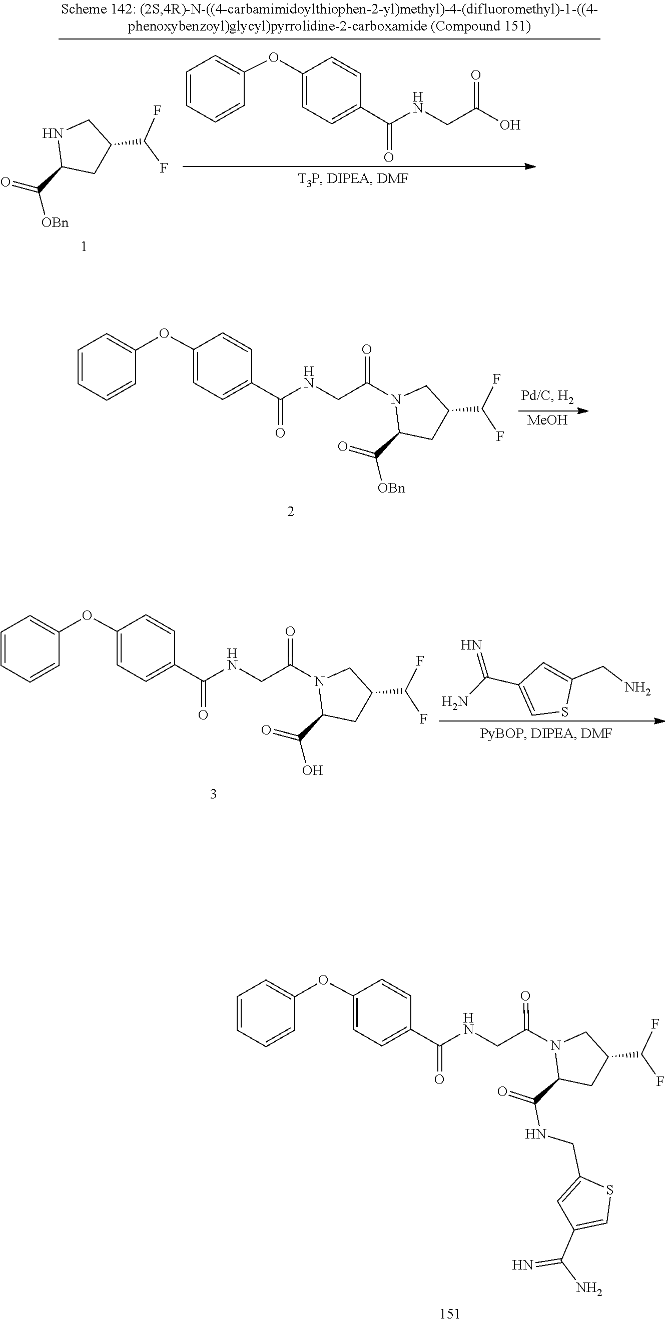

### Step 1: benzyl (2S,4R)-4-(difluoromethyl)-1-((4-phenoxybenzoyl)glycyl)pyrrolidine-2-carboxylate (2)

To a mixture of benzyl (2S,4R)-4-(difluoromethyl)pyrrolidine-2-carboxylate (106 mg, 0.42 mmol) and (4-phenoxybenzoyl)glycine (114 mg, 0.42 mmol) in DMF (2 mL) was added DIPEA (0.4 mL, 2.52 mmol) and T₃P (801 mg, 1.26 mmol, 50% wt in EtOAc) at 0° C. The resulting mixture was stirred at 35° C. for 5 hours. The mixture was washed with saturated aq.NaHCO₃, diluted with water and extracted with EtOAc twice. The combined organic layers were washed with brine, dried over anhydrous Na₂SO₄, filtered and concentrated to dryness under reduced pressure. The residue was purified by flash chromatography on silica gel (DCM:MeOH=20:1) to give benzyl (2S,4R)-4-(difluoromethyl)-1-((4-phenoxybenzoyl)glycyl)pyrrolidine-2-carboxylate (210 mg, yield 99.4%) as yellow oil. LC/MS (ESI) m/z: 509 (M+H)⁺

### Step 2: (2S,4R)-4-(difluoromethyl)-1-((4-phenoxybenzoyl)glycyl)pyrrolidine-2-carboxylic acid (3)

To a solution of benzyl (2S,4R)-4-(difluoromethyl)-1-((4-phenoxybenzoyl)glycyl)pyrrolidine-2-carboxylate (210 mg, 0.41 mmol) in MeOH (3 mL) were added Pd/C (21 mg, 10% wt) and the reaction mixture was stirred at room temperature under H₂ atmosphere for 2 hours. The mixture was filtered and concentrated to dryness under reduced pressure to give (2S,4R)-4-(difluoromethyl)-1-((4-phenoxybenzoyl)glycyl)pyrrolidine-2-carboxylic acid (144 mg, yield 83.3%) as light oil. LC/MS (ESI) m/z: 419 (M+H)⁺

### Step 3: (2S,4R)-N-((4-carbamimidoylthiophen-2-yl)methyl)-4-(difluoromethyl)-1-((4-phenoxybenzoyl)glycyl)pyrrolidine-2-carboxamide (Compound 151)

To a mixture of (2S,4R)-4-(difluoromethyl)-1-((4-phenoxybenzoyl)glycyl)pyrrolidine-2-carboxylic acid (67 mg, 0.16 mmol) and 5-(aminomethyl)thiophene-3-carboximidamide (25 mg, 0.16 mmol) in DMF (2 mL) was added DIPEA (0.10 mL, 0.80 mmol) and PyBOP (125 mg, 0.24 mmol) at 0° C. The mixture was stirred at 25° C. for half an hour and then extracted twice with EtOAc. The combined organic layers were washed with brine, dried over anhydrous Na₂SO₄, filtered and concentrated to dryness under reduced pressure. The residue was purified by prep-HPLC to give (2S,4R)-N-((4-carbamimidoylthiophen-2-yl)methyl)-4-(difluoromethyl)-1-((4-phenoxybenzoyl)glycyl)pyrrolidine-2-carboxamide (8.8 mg, yield 9.9%) as white solid. ¹H NMR (400 MHz, CD₃OD) δ 8.52 (s, 1H), 8.23 (dd, J=18.0, 18.0 Hz, 1H), 7.87-7.80 (m, 2H), 7.51-7.36 (m, 3H), 7.20 (t, J=7.4 Hz, 1H), 7.09-7.02 (m, 2H), 7.02-6.94 (m, 2H), 6.14-5.79 (m, 1H), 4.70-4.50 (m, 3H), 4.30-4.01 (m, 2H), 4.01-3.86 (m, 1H), 3.79-3.59 (m, 1H), 3.03-2.74 (m, 1H), 2.46-2.24 (m, 1H), 2.23-1.87 (m, 1H).LC/MS (ESI) m/z: 556 (M+H)⁺.

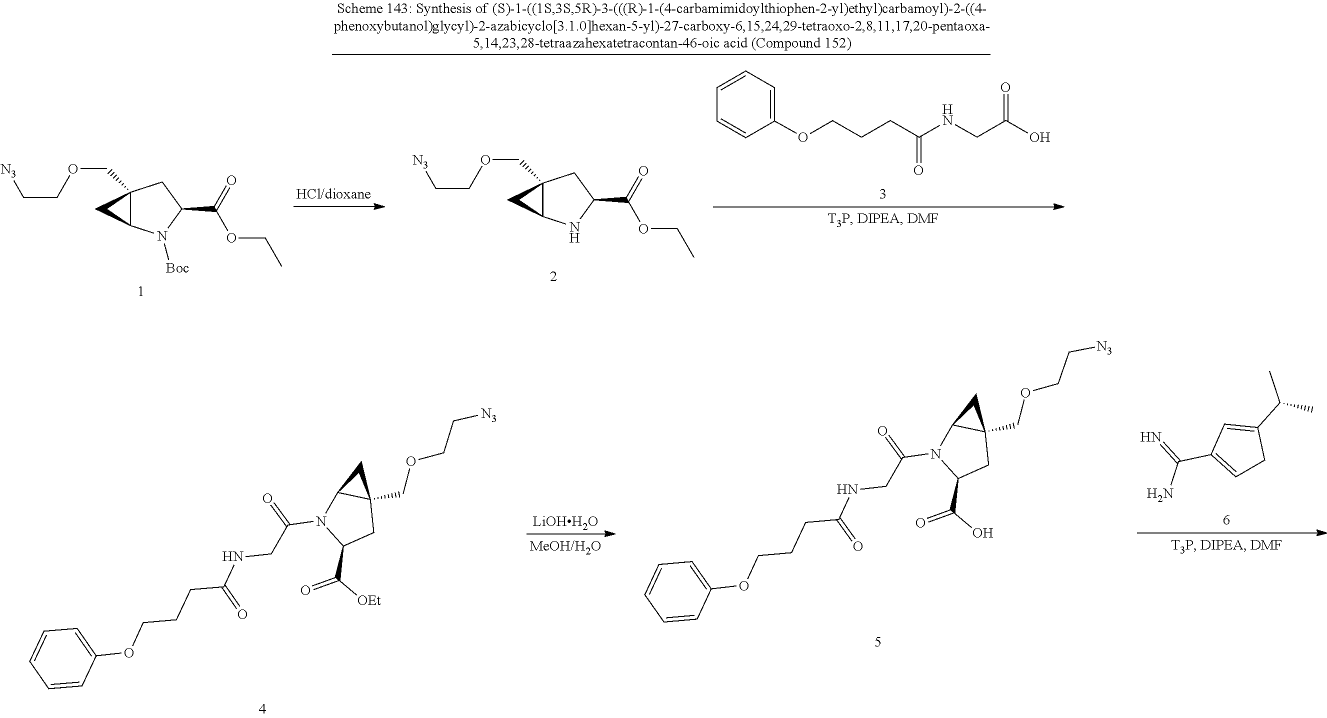

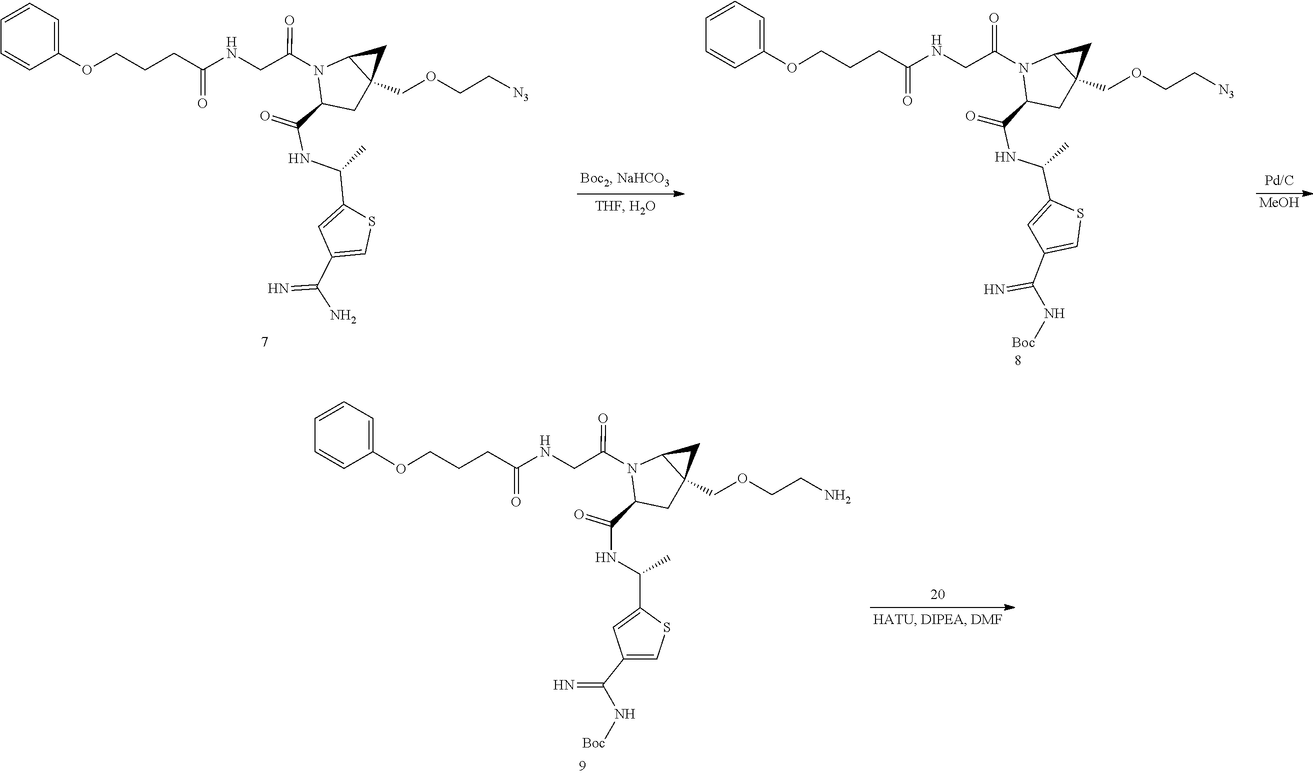 [SMILES: "MeOH" ; C[C@@H](NC(=O)[C@@H]1C[C@]2(COCCN=[N+]=[N-])C[C@@H]2N1C(=O)CNC(=O)CCCOc1ccccc1)c1cc(C(=N)N)cs1 ; C[C@@H](NC(=O)[C@@H]1C[C@]2(COCCN=[N+]=[N-])C[C@@H]2N1C(=O)CNC(=O)CCCOc1ccccc1)c1cc(C(=N)NC(=O)OC(C)(C)C)cs1 ; C[C@@H](NC(=O)[C@@H]1C[C@]2(COCCN)C[C@@H]2N1C(=O)CNC(=O)CCCOc1ccccc1)c1cc(C(=N)NC(=O)OC(C)(C)C)cs1]

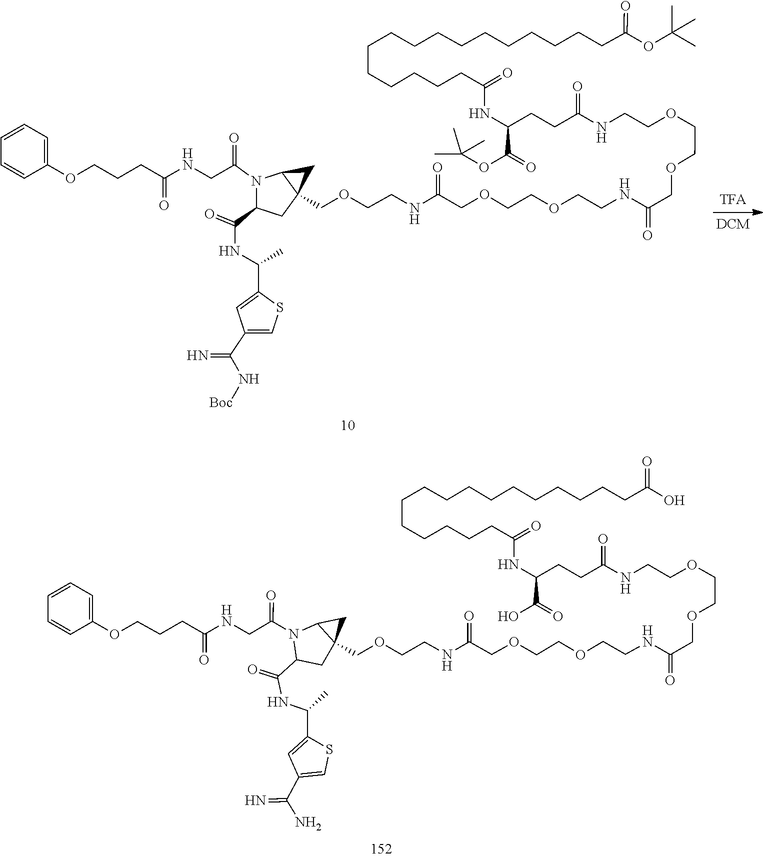

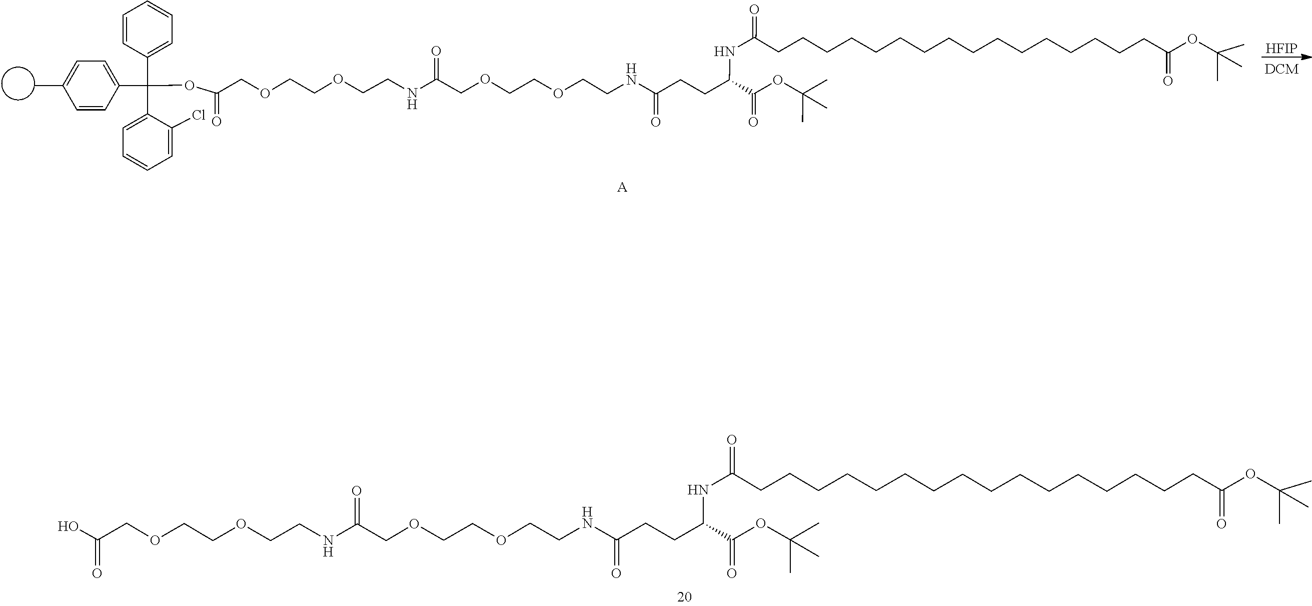

### Step 1: ethyl (1S,3S,5R)-5-((2-azidoethoxy)methyl)-2-azabicyclo[3.1.0]hexane-3-carboxylate (2)

A solution of 2-(tert-butyl) 3-ethyl (1 S,3S,5R)-5-((2-azidoethoxy)methyl)-2-azabicyclo[3.1.0]hexane-2,3-dicarboxylate (250 mg, 0.70 mmol) in HCl/1,4-dioxane (5 mL, 4 M) was stirred at room temperature for 1 hour. The reaction mixture was concentrated to dryness under reduced pressure, co-evaporated with DCM and dried under vacuum to give ethyl (1S,3S,5R)-5-((2-azidoethoxy)methyl)-2-azabicyclo[3.1.0]hexane-3-carboxylate (178 mg, yield 98.9%) as colorless oil, which was used directly in the next step without further purification. LC/MS (ESI) m/z: 255 (M+H)⁺.

### Step 2: ethyl (1S,3S,5R)-5-((2-azidoethoxy)methyl)-2-((4-phenoxybutanoyl)glycyl)-2-azabicyclo[3.1.0]hexane-3-carboxylate (4)

To a mixture of ethyl (1 S,3S,5R)-5-((2-azidoethoxy)methyl)-2-azabicyclo[3.1.0]hexane-3-carboxylate (178 mg, 0.70 mmol) and (4-phenoxybenzoyl)glycine (166 mg, 0.70 mmol) in DMF (5.0 mL) was added DIPEA (0.70 mL, 4.20 mmol) and T₃P (1.34 g, 2.1 mmol, 50% wt in EtOAc) at 0° C. under N₂ atmosphere and the mixture was stirred at 25° C. for 16 hours. The mixture was diluted with water and extracted with EtOAc twice. The combined organic layers were dried over anhydrous Na₂SO₄, filtered and concentrated to dryness under reduced pressure. The residue was purified by column chromatography on silica gel (DCM:MeOH=99:3) to give ethyl (1S,3S,5R)-5-((2-azidoethoxy)methyl)-2-((4-phenoxybutanoyl)glycyl)-2-azabicyclo[3.1.0]hexane-3-carboxylate (300 mg, yield 90.6%) as yellow oil. LC/MS (ESI) m/z: 474 (M+H)⁺.

### Step 3: (1S,3S,5R)-5-((2-azidoethoxy)methyl)-2-((4-phenoxybutanoyl)glycyl)-2-azabicyclo[3.1.0]hexane-3-carboxylic acid (5)

To a solution of ethyl (1 S,3S,5R)-5-((2-azidoethoxy)methyl)-2-((4-phenoxybutanoyl)glycyl)-2-azabicyclo[3.1.0]hexane-3-carboxylate (300 mg, 0.63 mmol) in MeOH (3 mL) and water (0.6 mL) was added lithium hydroxide (40 mg, 0.95 mmol) at 0° C. and the mixture was stirred at room temperature for 2 hours. The mixture was acidified with 1N aq.HCl to pH-3, extracted with CHCl₃/i-PrOH(3/1, v/v) five times. The combined organic layers were washed with brine, dried over anhydrous Na₂SO₄, filtered and concentrated to dryness under reduced pressure to give (1 S,3S,5R)-5-((2-azidoethoxy)methyl)-2-((4-phenoxybutanoyl)glycyl)-2-azabicyclo[3.1.0]hexane-3-carboxylic acid (250 mg, yield 88.7%) as light yellow oil, which was used directly in the next step without further purification. LC/MS (ESI) (m/z): 446 (M+H)⁺.

### Step 4: (1S,3S,5R)-5-((2-azidoethoxy)methyl)-N-((R)-1-(4-carbamimidoylthiophen-2-yl)ethyl)-2-((4-phenoxybutanoyl)glycyl)-2-azabicyclo[3.1.0]hexane-3-carboxamide (7)

To a mixture of (1 S,3S,5R)-5-((2-azidoethoxy)methyl)-2-((4-phenoxybutanoyl)glycyl)-2-azabicyclo[3.1.0]hexane-3-carboxylic acid (250 mg, 0.56 mmol) and (R)-5-(1-aminoethyl) thiophene-3-carboximidamide (142 mg, 0.84 mmol) in DMF (5.0 mL) was added DIPEA (0.56 mL, 3.36 mmol) and T₃P (1.07 g, 1.68 mmol, 50% wt in EtOAc) at 0° C. under N₂ atmosphere and the mixture was stirred at 25° C. for 16 hours. The mixture was diluted with water and extracted with EtOAc twice. The combined organic layers were dried over anhydrous Na₂SO₄, filtered and concentrated to dryness under reduced pressure. The residue was purified by pre-TLC (DCM:MeOH=10:1) to give (1S,3S,5R)-5-((2-azidoethoxy)methyl)-N-((R)-1-(4-carbamimidoylthiophen-2-yl)ethyl)-2-((4-phenoxybutanoyl)glycyl)-2-azabicyclo[3.1.0]hexane-3-carboxamide (110 mg, yield 34.1%) as white solid. LC/MS (ESI) m/z: 597 (M+H)⁺.

### Step 5: tert-butyl ((5-((R)-1-((1S,3S,5R)-5-((2-azidoethoxy)methyl)-2-((4-phenoxybutanoyl) glycyl)-2-azabicyclo[3.1.0]hexane-3-carboxamido)ethyl)thiophen-3-yl)(imino)methyl) carbamate (8)

To a solution of (1 S,3S,5R)-5-((2-azidoethoxy)methyl)-N-((R)-1-(4-carbamimidoylthiophen-2-yl)ethyl)-2-((4-phenoxybutanoyl)glycyl)-2-azabicyclo[3.1.0]hexane-3-carboxamide (110 mg, 0.18 mmol) in THE (5.0 mL) was added NaHCO₃ (154 mg, 1.80 mmol) in water (5.0 mL), followed by drop-wise addition of BoC₂O (121 mg, 0.54 mmol) at 0° C. and the mixture was stirred at 25° C. for 16 hours. The mixture was diluted with EtOAc and washed with brine, dried over anhydrous Na₂SO₄, filtered and concentrated to dryness under reduced pressure. The residue was purified by pre-TLC (DCM:MeOH=10:1) to give tert-butyl ((5-((R)-1-((1S,3S,5R)-5-((2-azidoethoxy)methyl)-2-((4-phenoxybutanoyl)glycyl)-2-azabicyclo[3.1.0]hexane-3-carboxamido)ethyl)thiophen-3-yl)(imino)methyl)carbamate (80 mg, yield 62.5%) as white solid. LC/MS (ESI) m/z: 697 (M+H)⁺.

### Step 6: tert-butyl ((5-((R)-1-((1S,3S,5R)-5-((2-aminoethoxy)methyl)-2-((4-phenoxybutanoyl)glycyl)-2-azabicyclo[3.1.0]hexane-3-carboxamido)ethyl)thiophen-3-yl)(imino)methyl)carbamate (9)

To a solution of tert-butyl ((5-((R)-1-((1 S,3S,5R)-5-((2-azidoethoxy)methyl)-2-((4-phenoxybutanoyl)glycyl)-2-azabicyclo[3.1.0]hexane-3-carboxamido)ethyl)thiophen-3-yl)(imino)methyl)carbamate (80 mg, 0.11 mmol) in MeOH (5.0 mL) was added Pd/C (20 mg, 10% wt) and the mixture was degassed under N₂ atmosphere for three times and stirred under a H₂ balloon at room temperature for 1 hour. The mixture was filtered and filtrate was concentrated to dryness under reduced pressure to give tert-butyl ((5-((R)-1-((1 S,3S,5R)-5-((2-aminoethoxy)methyl)-2-((4-phenoxybutanoyl)glycyl)-2-azabicyclo[3.1.0]hexane-3-carboxamido)ethyl)thiophen-3-yl)(imino)methyl)carbamate (53.0 mg, yield 68.8%) as white solid, which was used directly in next step without further purification. LC/MS (ESI) (m/z): 671 (M+H)⁺.

### Step 7: tert-butyl (S)-27-(tert-butoxycarbonyl)-1-((1S,3S,5R)-3-(((R)-1-(4-(N-(tert-butoxycarbonyl)carbamimidoyl)thiophen-2-yl)ethyl)carbamoyl)-2-((4-phenoxybutanoyl) glycyl)-2-azabicyclo[3.1.0]hexan-5-yl)-6,15,24,29-tetraoxo-2,8,11,17,20-pentaoxa-5,14,23,28-tetraazahexatetracontan-46-oate (10)

To a mixture of tert-butyl ((5-((R)-1-((1 S,3S,5R)-5-((2-aminoethoxy)methyl)-2-((4-phenoxybutanoyl)glycyl)-2-azabicyclo[3.1.0]hexane-3-carboxamido)ethyl)thiophen-3-yl)(imino)methyl)carbamate (50 mg, 0.07 mmol) and (S)-22-(tert-butoxycarbonyl)-43,43-dimethyl-10,19,24,41-tetraoxo-3,6,12,15,42-pentaoxa-9,18,23-triazatetratetracontanoic acid (95 mg, 0.11 mmol) in DMF (3.0 mL) was added DIPEA (0.07 mL, 0.42 mmol) and HATU (43 mg, 0.11 mmol) at 0° C. under N₂ atmosphere and the mixture was stirred at 25° C. for 1 hour. The mixture was diluted with water and extracted with EtOAc twice. The combined organic layers were dried over anhydrous Na₂SO₄, filtered and concentrated to dryness under reduced pressure. The residue was purified by pre-TLC (DCM:MeOH=10:1) to give tert-butyl (S)-27-(tert-butoxycarbonyl)-1-((1S,3S,5R)-3-(((R)-1-(4-(N-(tert-butoxycarbonyl)carbamimidoyl) thiophen-2-yl)ethyl)carbamoyl)-2-((4-phenoxybutanoyl)glycyl)-2-azabicyclo[3.1.0]hexan-5-yl)-6,15,24,29-tetraoxo-2,8,11,17,20-pentaoxa-5,14,23,28-tetraazahexatetracontan-46-oate (50 mg, yield 45.0%) as white solid. LC/MS (ESI) m/z: 1499 (M+H)⁺.

### Step 8: (S)-1-((1S,3S,5R)-3-(((R)-1-(4-carbamimidoylthiophen-2-yl)ethyl)carbamoyl)-2-((4-phenoxybutanoyl)glycyl)-2-azabicyclo[3.1.0]hexan-5-yl)-27-carboxy-6,15,24,29-tetraoxo-2,8,11,17,20-pentaoxa-5,14,23,28-tetraazahexatetracontan-46-oic acid (Compound 152)

A solution of tert-butyl (S)-27-(tert-butoxycarbonyl)-1-((1S,3S,5R)-3-(((R)-1-(4-(N-(tert-butoxycarbonyl)carbamimidoyl)thiophen-2-yl)ethyl)carbamoyl)-2-((4-phenoxybutanoyl)glycyl)-2-azabicyclo[3.1.0]hexan-5-yl)-6,15,24,29-tetraoxo-2,8,11,17,20-pentaoxa-5,14,23,28-tetraazahexatetracontan-46-oate (50 mg, 0.03 mmol) in DCM (2 mL) was added TFA (1 mL) and the solution was stirred at room temperature for 1 hour. The reaction mixture was concentrated to dryness under reduced pressure, co-evaporated with DCM and dried under vacuum. The residue was purified by prep-HPLC to give (S)-1-((1S,3S,5R)-3-(((R)-1-(4-carbamimidoylthiophen-2-yl)ethyl)carbamoyl)-2-((4-phenoxybutanoyl)glycyl)-2-azabicyclo[3.1.0]hexan-5-yl)-27-carboxy-6,15,24,29-tetraoxo-2,8,11,17,20-pentaoxa-5,14,23,28-tetraazahexatetracontan-46-oic acid (8.0 mg, yield 18.6%) as white solid. ¹H NMR (400 MHz, CD₃OD) δ 8.41 (s, 1H), 8.23 (d, J=1.6 Hz, 1H), 7.53 (d, J=6.6 Hz, 1H), 7.29-7.19 (m, 2H), 6.94-6.86 (m, 3H), 5.20 (q, J=6.8 Hz, 1H), 4.33-4.20 (m, 2H), 4.12 (d, J=16.6 Hz, 1H), 4.00 (t, J=5.2 Hz, 6H), 3.69-3.62 (m, 8H), 3.61-3.52 (m, 8H), 3.48-3.42 (m, 5H), 3.36 (dd, J=9.3, 5.6 Hz, 2H), 2.65 (t, J=12.4 Hz, 1H), 2.48 (t, J=7.4 Hz, 2H), 2.25 (dt, J=12.1, 7.8 Hz, 6H), 2.14-2.03 (m, 4H), 1.95 (dd, J=14.4, 7.9 Hz, 1H), 1.63-1.54 (m, 7H), 1.30 (d, J=10.7 Hz, 25H), 1.25 (d, J=2.6 Hz, 1H), 0.99 (t, J=5.7 Hz, 1H). LC/MS (ESI) m/z: 1287 (M+H)⁺.

### Step 9: Synthesis of (S)-22-(tert-butoxycarbonyl)-43,43-dimethyl-10,19,24,41-tetraoxo-3,6,12,15,42-pentaoxa-9,18,23-triazatetratetracontanoic acid (20)

Compound A (200 mg, 0.18 mmol) was treated with 20% HFIP in DCM (3 mL) and shaken for 3 hours at room temperature. The mixture was filtered and the solution was washed with 20% HFIP in DCM (0.3 mL) and then concentrated to give (S)-22-(tert-butoxycarbonyl)-43,43-dimethyl-10,19,24,41-tetraoxo-3,6,12,15,42-pentaoxa-9,18,23-triazatetratetracontanoic acid (140 mg, 94.0% yield) as colorless oil. LC/MS(ESI) m/z: 847 (M+H)⁺.

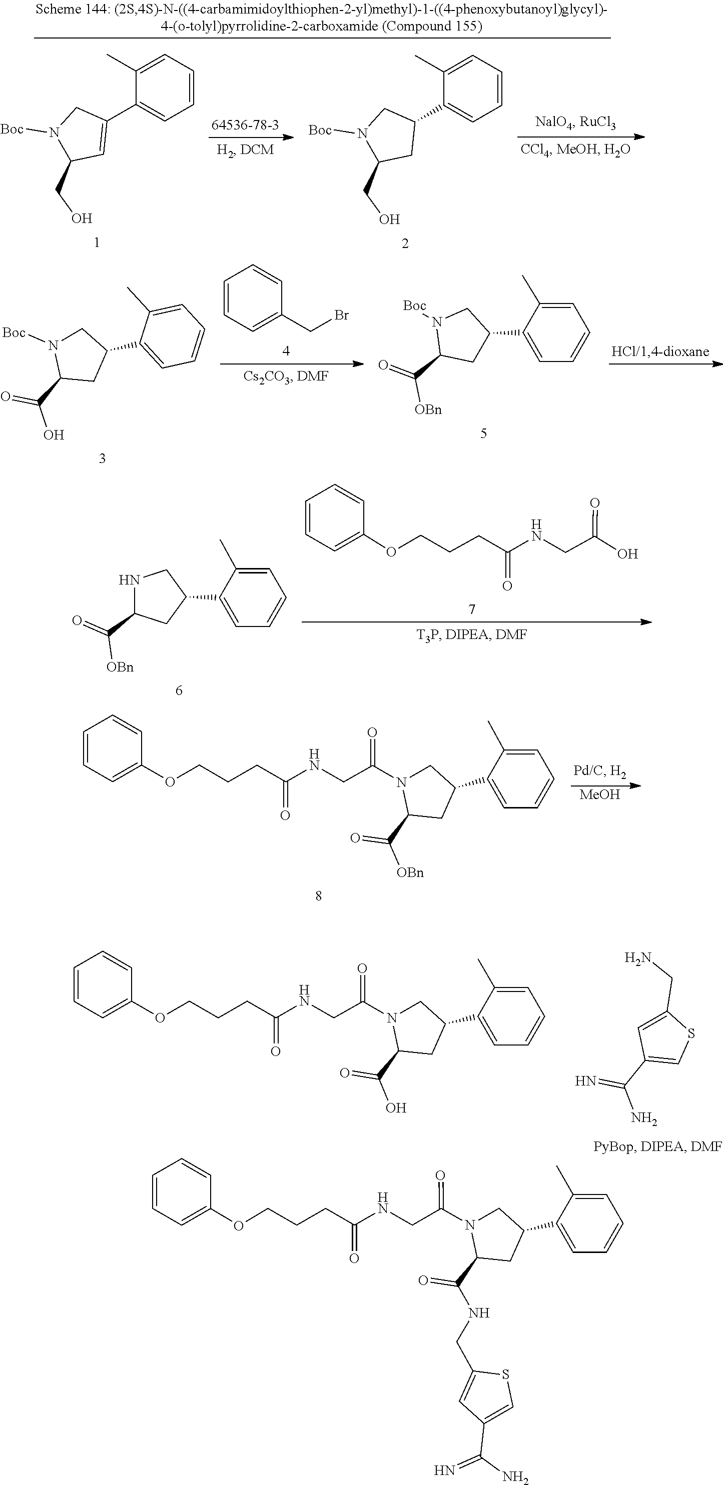

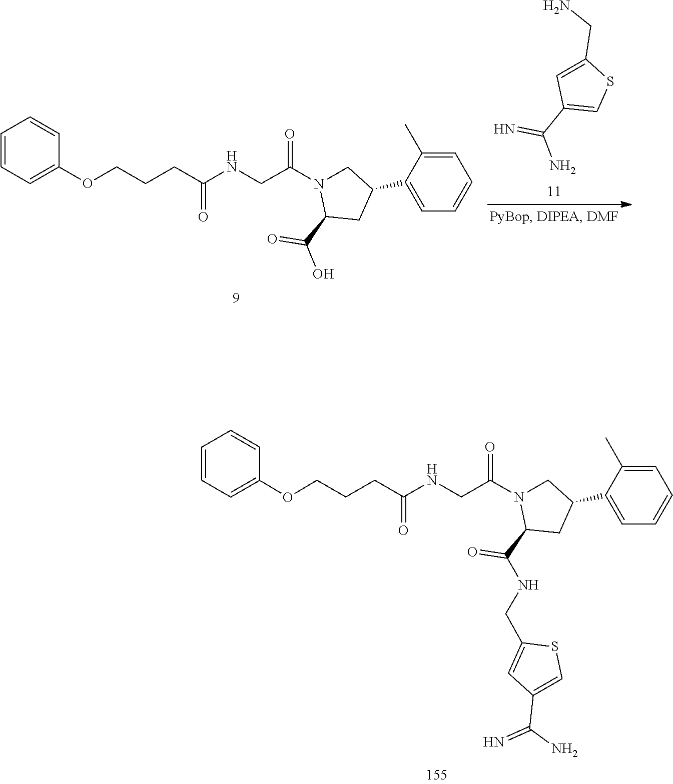 [SMILES: Cc1ccccc1[C@@H]1C[C@@H](C(=O)O)N(C(=O)CNC(=O)CCCOc2ccccc2)C1 ; N=C(N)c1csc(CN)c1 ; Cc1ccccc1[C@@H]1C[C@@H](C(=O)NCc2cc(C(=N)N)cs2)N(C(=O)CNC(=O)CCCOc2ccccc2)C1]

### Step 1: tert-butyl (2S,4S)-2-(hydroxymethyl)-4-(o-tolyl)pyrrolidine-1-carboxylate

To a solution of tert-butyl (S)-2-(hydroxymethyl)-4-(o-tolyl)-2,5-dihydro-1H-pyrrole-1-carboxylate (110 mg, 0.38 mmol) in DCM (1.4 ml) was added (1,5-cyclooctadiene) (pyridine)(tricyclohexylphosphine)iridium(I) hexafluorophosphate (CAS #64536-78-3) (20 mg, 0.025 mmol) under a H₂ balloon. The mixture was stirred for 4 hours before diluted with water and extracted with ethyl acetate twice. The combined organic layers were washed with brine, dried over anhydrous Na₂SO₄, filtered and concentrated to dryness under reduced pressure. The residue was purified by column chromatography on silica gel (PE:Ethyl Acetate=3:1) to give the title compound tert-butyl (2S,4S)-2-(hydroxymethyl)-4-(o-tolyl)pyrrolidine-1-carboxylate (60 mg, yield 54.2%) as white solid. LCMS (ESI) m/z=292 (M+H)⁺.

### Step 2: (2S,4S)-1-(tert-butoxycarbonyl)-4-(o-tolyl)pyrrolidine-2-carboxylic acid

To a mixture of tert-butyl (2S,4S)-2-(hydroxymethyl)-4-(o-tolyl)pyrrolidine-1-carboxylate (112 mg, 0.38 mmol) and NaIO₄ (245 mg, 1.15 mmol) in CCl₄ (0.5 ml)/CH₃CN (0.5 ml)/H₂O (0.8 ml) under N₂ atmosphere at room temperature was added RuCl₃ (3.0 mg, 0.014 mmol). The mixture was stirred for 4 hours before diluted with water and extracted with ethyl acetate twice. The combined organic layers were washed with brine, dried over anhydrous Na₂SO₄, filtered and concentrated to dryness under reduced pressure. The residue was purified by column chromatography on silica gel (DCM:MeOH=10:1) to give the title compound (2S,4S)-1-(tert-butoxycarbonyl)-4-(o-tolyl)pyrrolidine-2-carboxylic acid (60 mg, yield 51.2%) as white solid. LCMS (ESI) m/z=250 (M+H−56)⁺.

### Step 3: 2-benzyl 1-(tert-butyl) (2S,4S)-4-(o-tolyl)pyrrolidine-1,2-dicarboxylate

To a mixture of (2S,4S)-1-(tert-butoxycarbonyl)-4-(o-tolyl)pyrrolidine-2-carboxylic acid (127 mg, 0.42 mmol) and Cs₂CO₃ (149 mg, 0.46 mmol) in DMF (1.2 ml) was added benzyl bromide (85 mg, 0.50 mmol) at room temperature under N₂ atmosphere and the mixture was stirred overnight. The mixture was diluted with water and extracted with ethyl acetate twice. The combined organic layers were washed with brine, dried over anhydrous Na₂SO₄, filtered and concentrated to dryness under reduced pressure. The residue was purified by column chromatography on silica gel (PE:Ethyl Acetate=3:1) to give 2-benzyl 1-(tert-butyl) (2S,4S)-4-(o-tolyl)pyrrolidine-1,2-dicarboxylate (30 mg, yield 18%) as white solid. LCMS (ESI) m/z=396 (M+H)⁺.

### Step 4: benzyl (2S,4S)-4-(o-tolyl)pyrrolidine-2-carboxylate

To a mixture of 2-benzyl 1-(tert-butyl) (2S,4S)-4-(o-tolyl)pyrrolidine-1,2-dicarboxylate (30 mg, 0.068 mmol) in HCl/1,4-dioxane (2 mL) was stirred at room temperature for 2 hours. The reaction mixture was concentrated to dryness under reduced pressure, diluted with DCM and dried under vacuum to give benzyl (2S,4S)-4-(o-tolyl)pyrrolidine-2-carboxylate (20 mg, yield 89.2%), which was used directly in the next step without further purification. LC/MS (ESI) m/z: 296 (M+H)⁺.

### Step 5: benzyl (2S,4S)-1-((4-phenoxybutanoyl)glycyl)-4-(o-tolyl)pyrrolidine-2-carboxylate

To a mixture of benzyl (2S,4S)-4-(o-tolyl)pyrrolidine-2-carboxylate (20 mg, 0.068 mmol) and (4-phenoxybutanoyl)glycine (24 mg, 0.10 mmol) in DMF (0.8 mL) was added DIPEA (52 mg, 0.41 mmol) and T₃P (64 mg, 0.20 mmol, 50% in ethyl acetate) at 0° C. and the mixture was stirred under N₂ atmosphere at room temperature for 2 hours. The organic layer was diluted with water extracted with ethyl acetate twice and the combined organic layers were washed with brine, dried over anhydrous Na₂SO₄, filtered and concentrated to dryness under reduced pressure. The residue was purified by flash chromatography on silica gel (petroleum ether:ethyl acetate=3:1) to give benzyl (2S,4S)-1-((4-phenoxybutanoyl)glycyl)-4-(o-tolyl)pyrrolidine-2-carboxylate (12 mg, yield 34.5%) as white solid. LC/MS (ESI) (m/z): 515(M+H)⁺.

### Step 6: (2S,4S)-1-((4-phenoxybutanoyl)glycyl)-4-(o-tolyl)pyrrolidine-2-carboxylic acid

To a solution of benzyl (2S,4S)-1-((4-phenoxybutanoyl)glycyl)-4-(o-tolyl)pyrrolidine-2-carboxylate (12 mg, 0.023 mmol) in MeOH (1.0 mL) was added Pd/C (10 mg, 10% wt) at room temperature and the mixture was stirred under a H₂ balloon for 2 hours. The mixture was filtered and the filtrate was concentrated to dryness under reduced pressure to give (2S,4S)-1-((4-phenoxybutanoyl)glycyl)-4-(o-tolyl)pyrrolidine-2-carboxylic acid (4 mg, yield 41.0%) as colorless oil, which was used directly in the next step without further purification. LC/MS (ESI) (m/z): 425(M+H)⁺.

### Step 7: (2S,4S)-N-((4-carbamimidoylthiophen-2-yl)methyl)-1-((4-phenoxybutanoyl)glycyl)-4-(o-tolyl)pyrrolidine-2-carboxamide (Compound 155)

To a mixture of (2S,4S)-1-((4-phenoxybutanoyl)glycyl)-4-(o-tolyl)pyrrolidine-2-carboxylic acid (4 mg, 0.0094 mmol) and 5-(aminomethyl)thiophene-3-carboximidamide (2 mg, 0.014 mmol) in DMF (0.8 mL) was added DIPEA (7 mg, 0.057 mmol) and PyBop (7 mg, 0.014 mmol) at 0° C. and the mixture was stirred under N₂ atmosphere at room temperature for 2 hours. The organic layer was diluted with water, extracted with ethyl acetate twice and the combined organic layers were washed with brine, dried over anhydrous Na₂SO₄, filtered and concentrated to dryness under reduced pressure. The residue was purified by flash chromatography on silica gel (petroleum ether:ethyl acetate=3:1) and prep-HPLC to give Compound 155 (2 mg, yield 37.8%) as a white solid. ¹H NMR (400 MHz, CD₃OD) δ 8.48 (s, 1H), 8.23 (d, J=1.6 Hz, 1H), 7.47 (d, J=21.9 Hz, 1H), 7.25 (dd, J=20.4, 12.7 Hz, 3H), 7.20-7.13 (m, 3H), 6.89 (dd, J=7.9, 4.1 Hz, 3H), 4.68-4.62 (m, 2H), 4.56 (d, J=15.6 Hz, 2H), 4.15-4.04 (m, 2H), 4.01 (dd, J=11.4, 5.1 Hz, 3H), 3.82 (d, J=9.8 Hz, 1H), 3.59 (t, J=9.7 Hz, 1H), 2.47 (t, J=7.4 Hz, 2H), 2.41-2.36 (m, 1H), 2.31 (d, J=15.4 Hz, 3H), 2.11-2.05 (m, 2H). LCMS (ESI) m/z=562(M+H)⁺.

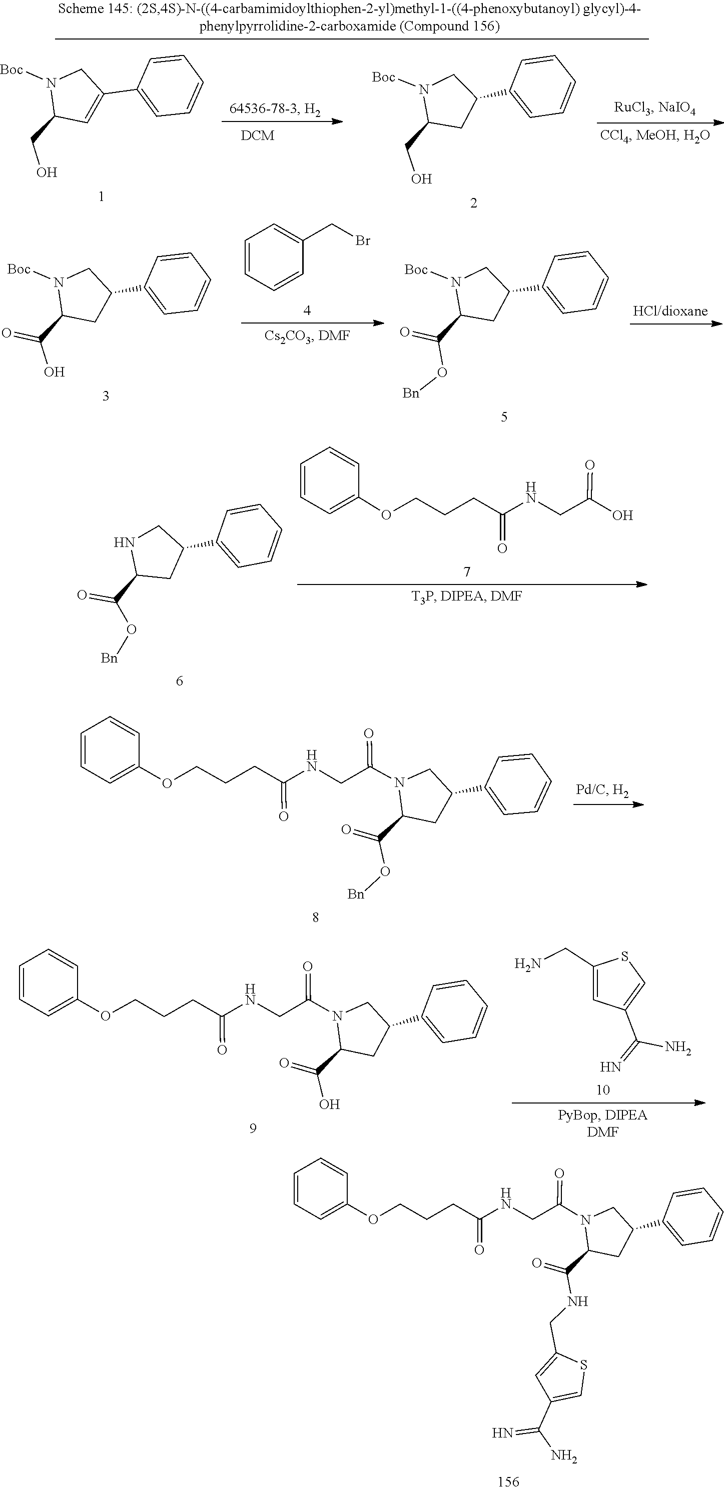

### Step 1: tert-butyl (2S,4S)-2-(hydroxymethyl)-4-phenylpyrrolidine-1-carboxylate

To a solution of tert-butyl (S)-2-(hydroxymethyl)-4-phenyl-2,5-dihydro-1H-pyrrole-1-carboxylate (200 mg, 0.72 mmol) in DCM (5 mL) was added (1,5-cyclooctadiene) (pyridine)(tricyclohexylphosphine)iridium(I) hexafluorophosphate (57 mg, 0.07 mmol) at room temperature and the mixture was stirred under a H₂ balloon at room temperature for 12 hours. The mixture was filtered and the filtrate was concentrated to dryness under reduced pressure to give tert-butyl (2S,4S)-2-(hydroxymethyl)-4-phenylpyrrolidine-1-carboxylate (200 mg, yield 99.8%) as a white solid, which was used directly in the next step without further purification. LC/MS (ESI) (m/z): 278(M+H)⁺.

### Step 2: (2S,4S)-1-(tert-butoxycarbonyl)-4-phenylpyrrolidine-2-carboxylic acid

To a mixture of tert-butyl (2S,4S)-2-(hydroxymethyl)-4-phenylpyrrolidine-1-carboxylate (200 mg, 0.72 mmol) and NaIO₄ (462 mg, 2.16 mmol) in CCl₄ (0.8 ml)/CH₃CN (0.8 ml)/water (1.2 ml) was stirred under N₂ atmosphere at room temperature for 20 minutes before RuCl₃ (15 mg, 0.072 mmol) was added. The mixture was stirred for 4 hours before diluted with water and extracted with ethyl acetate twice. The combined organic layers were washed with brine, dried over anhydrous Na₂SO₄, filtered and concentrated to dryness under reduced pressure. The residue was purified by column chromatography on silica gel (PE:Ethyl Acetate=3:1) to give the title compound (2S,4S)-1-(tert-butoxycarbonyl)-4-phenylpyrrolidine-2-carboxylic acid (120 mg, yield 57%) as a colorless oil. LCMS (ESI) m/z=292(M+H)⁺.

### Step 3: 2-benzyl 1-(tert-butyl) (2S,4S)-4-phenylpyrrolidine-1,2-dicarboxylate

To a mixture of (2S,4S)-1-(tert-butoxycarbonyl)-4-phenylpyrrolidine-2-carboxylic acid (120 mg, 0.41 mmol) and Cs₂CO₃ (400 mg, 1.22 mmol) in DMF (6 ml) was added benzyl bromide (76 mg, 0.44 mmol) under N₂ atmosphere at room temperature and the reaction mixture was stirred overnight. The mixture was diluted with water and extracted with ethyl acetate twice. The combined organic layers were washed with brine, dried over anhydrous Na₂SO₄, filtered and concentrated to dryness under reduced pressure. The residue was purified by column chromatography on silica gel (PE:Ethyl Acetate=3:1) to give 2-benzyl 1-(tert-butyl) (2S,4S)-4-phenylpyrrolidine-1,2-dicarboxylate (150 mg, yield 96%) as a white solid. LCMS (ESI) m/z=382(M+H)⁺.

### Step 4: benzyl (2S,4S)-4-phenylpyrrolidine-2-carboxylate

To a mixture of 2-benzyl 1-(tert-butyl) (2S,4S)-4-phenylpyrrolidine-1,2-dicarboxylate (150 mg, 0.38 mmol) in HCl/1,4-dioxane (3 mL) was stirred under N₂ atmosphere at room temperature for 2 hours. The reaction mixture was concentrated to dryness under reduced pressure, diluted with DCM and dried under vacuum to give benzyl (2S,4S)-4-phenylpyrrolidine-2-carboxylate (100 mg, quantitative) as a yellow solid, which was used directly in the next step without further purification. LC/MS (ESI) m/z: 282(M+H)⁺.

### Step 5: benzyl (2S,4S)-1-((4-phenoxybutanoyl)glycyl)-4-phenylpyrrolidine-2-carboxylate

To a mixture of benzyl (2S,4S)-4-phenylpyrrolidine-2-carboxylate (100 mg, 0.35 mmol) and (4-phenoxybutanoyl)glycine (78 mg, 0.32 mmol) in DMF (4 mL) was added DIPEA (123 mg, 1.98 mmol) and T₃P (1.22 g, 1.92 mmol, 50% in ethyl acetate) at 0° C. and the mixture was stirred under N₂ atmosphere at room temperature for 2 hours. The organic layer was diluted with water extracted with ethyl acetate twice and the combined organic layers were washed with brine, dried over anhydrous Na₂SO₄, filtered and concentrated to dryness under reduced pressure. The residue was purified by flash chromatography on silica gel (petroleum ether:ethyl acetate=1:1) to give benzyl (2S,4S)-1-((4-phenoxybutanoyl)glycyl)-4-phenylpyrrolidine-2-carboxylate (100 mg, yield 62%) as white solid. LC/MS (ESI) (m/z): 501(M+H)⁺.

### Step 6: (2S,4S)-1-((4-phenoxybutanoyl)glycyl)-4-phenylpyrrolidine-2-carboxylic acid

To a solution of benzyl (2S,4S)-1-((4-phenoxybutanoyl)glycyl)-4-phenylpyrrolidine-2-carboxylate (100 mg, 0.20 mmol) in MeOH (4 mL) was added Pd/C (10 mg, 10% wt) at room temperature and the mixture was stirred under a H₂ balloon for 2 hours. The mixture was filtered and the filtrate was concentrated to dryness under reduced pressure to give (2S,4S)-1-((4-phenoxybutanoyl)glycyl)-4-phenylpyrrolidine-2-carboxylic acid (80 mg, yield 97%) as a colorless oil, which was used directly in the next step without further purification. LC/MS (ESI) (m/z): 411 (M+H)⁺.

### Step 7: (2S,4S)-N-((4-carbamimidoylthiophen-2-yl)methyl)-1-((4-phenoxybutanoyl)glycyl)-4-phenylpyrrolidine-2-carboxamide (Compound 156)

To a mixture of (2S,4S)-1-((4-phenoxybutanoyl)glycyl)-4-phenylpyrrolidine-2-carboxylic acid (40 mg, 0.10 mmol) and 5-(aminomethyl)thiophene-3-carboximidamide (16 mg, 0.10 mmol) in DMF (1 mL) was added DIPEA (40 mg, 0.31 mmol) and PyBop (100 mg, 0.20 mmol) at 0° C. and the mixture was stirred under N₂ atmosphere at room temperature for 2 hours. The organic layer was diluted with water, extracted with ethyl acetate twice and the combined organic layers were washed with brine, dried over anhydrous Na₂SO₄, filtered and concentrated to dryness under reduced pressure. The residue was purified by prep-HPLC to afford (2S,4S)-N-((4-carbamimidoylthiophen-2-yl)methyl)-1-((4-phenoxybutanoyl) glycyl)-4-phenylpyrrolidine-2-carboxamide (12.4 mg, yield 22.6%) as a white solid. ¹HNMR (400 MHz, CD₃OD) δ 8.55 (s, 1H), 8.24 (dd, J=5.3, 1.6 Hz, 1H), 7.47 (d, J=27.0 Hz, 1H), 7.38-7.29 (m, 4H), 7.24 (dd, J=9.7, 6.3 Hz, 3H), 6.91 (dd, J=8.0, 3.2 Hz, 3H), 4.73-4.65 (m, 1H), 4.61 (s, 2H), 4.18-4.05 (m, 2H), 4.05-3.98 (m, 3H), 3.62 (d, J=3.1 Hz, 2H), 2.48 (t, J=7.4 Hz, 2H), 2.46-2.32 (m, 2H), 2.12-2.04 (m, 2H)., LC/MS (ESI) (m/z): 548 (M+H)⁺.

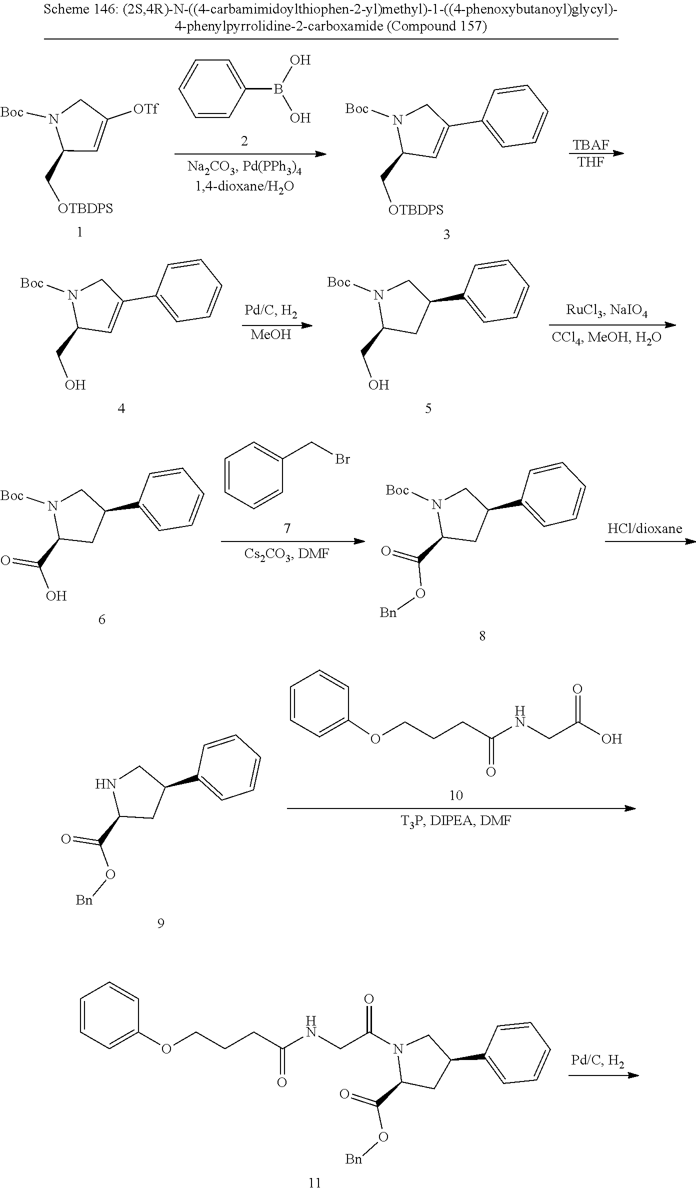

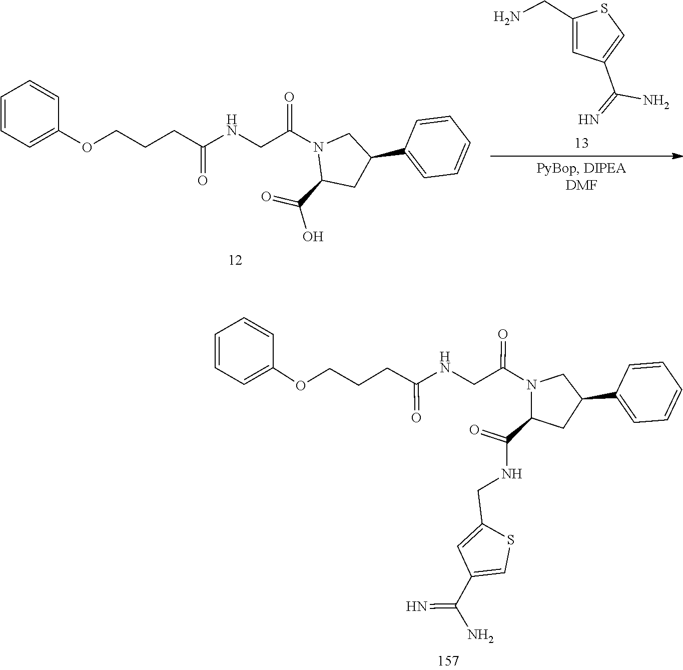 [SMILES: O=C(CCCOc1ccccc1)NCC(=O)N1C[C@@H](c2ccccc2)C[C@H]1C(=O)O ; N=C(N)c1csc(CN)c1 ; N=C(N)c1csc(CNC(=O)[C@@H]2C[C@H](c3ccccc3)CN2C(=O)CNC(=O)CCCOc2ccccc2)c1]

### Step 1: tert-butyl (S)-2-(((tert-butyldiphenylsilyl)oxy)methyl)-4-(4-fluorophenyl)-2,5-dihydro-1H-pyrrole-1-carboxylate

To a mixture of tert-butyl (S)-2-(((tert-butyldiphenylsilyl)oxy)methyl)-4-(((trifluoromethyl)sulfonyl)oxy)-2,5-dihydro-1H-pyrrole-1-carboxylate (1.0 g, 1.7 mmol)) and phenylboronic acid (230 mg, 1.87 mmol) in 1,4-dioxane (10 ml) and water (3 ml) was added Na₂CO₃ (540 mg, 5.1 mmol) and Pd(PPh₃)₄ (200 mg, 0.17 mmol) under N₂ atmosphere and the mixture was stirred at 80° C. overnight. The mixture was diluted with water and extracted with ethyl acetate twice. The combined organic layers were washed with brine, dried over anhydrous Na₂SO₄, filtered and concentrated to dryness under reduced pressure. The residue was purified by column chromatography on silica gel (PE:EA=10:1) to give tert-butyl (S)-2-(((tert-butyldiphenylsilyl)oxy)methyl)-4-phenyl-2,5-dihydro-1H-pyrrole-1-carboxylate (800 mg, yield 91.7%) as a white solid. LCMS (ESI) m/z=514 (M+H)⁺.

### Step 2: tert-butyl (S)-2-(hydroxymethyl)-4-phenyl-2,5-dihydro-1H-pyrrole-1-carboxylate

To a solution of tert-butyl (S)-2-(((tert-butyldiphenylsilyl)oxy)methyl)-4-phenyl-2,5-dihydro-1H-pyrrole-1-carboxylate (800 mg, 1.56 mmol) in THE (3 mL) was added TBAF (1.22 g, 4.68 mmol) at 0° C. and the mixture was stirred at room temperature for 2 hours. The mixture was diluted with saturated aq.NH₄Cl solution and extracted with ethyl acetate twice. The combined organic layers were washed with brine, dried over anhydrous Na₂SO₄, filtered and concentrated to dryness under reduced pressure. The residue was purified by column chromatography on silica gel (PE:Ethyl Acetate=10:1) to give tert-butyl (S)-2-(hydroxymethyl)-4-phenyl-2,5-dihydro-1H-pyrrole-1-carboxylate (400 mg, yield 93%) as a white solid. LCMS (ESI) m/z=276(M+H)⁺.

### Step 3: tert-butyl (2S,4R)-2-(hydroxymethyl)-4-phenylpyrrolidine-1-carboxylate

To a solution of tert-butyl (S)-2-(hydroxymethyl)-4-phenyl-2,5-dihydro-1H-pyrrole-1-carboxylate (200 mg, 0.72 mmol) in MeOH (5 mL) was added Pd/C (20 mg, 10% wat) at room temperature and the mixture was stirred under a H₂ balloon at room temperature for 2 hours. The mixture was filtered and the filtrate was concentrated to dryness under reduced pressure to give tert-butyl (2S,4R)-2-(hydroxymethyl)-4-phenylpyrrolidine-1-carboxylate (200 mg, yield 99.8%) as colorless oil, which was used directly in the next step without further purification. LC/MS (ESI) (m/z): 278(M+H)⁺.

### Step 4: (2S,4R)-1-(tert-butoxycarbonyl)-4-phenylpyrrolidine-2-carboxylic acid

To a mixture of tert-butyl (2S,4R)-2-(hydroxymethyl)-4-phenylpyrrolidine-1-carboxylate (200 mg, 0.72 mmol) and NaIO₄ (462 mg, 2.16 mmol) in CCl₄ (0.8 ml)/CH₃CN (0.8 ml)/water (1.2 ml) was stirred under N₂ atmosphere at room temperature for 20 minutes before RuCl₃ (15 mg, 0.072 mmol) was added. The mixture was stirred for 4 hours before diluted with water and extracted with ethyl acetate twice. The combined organic layers were washed with brine, dried over anhydrous Na₂SO₄, filtered and concentrated to dryness under reduced pressure. The residue was purified by flash column chromatography on silica gel (PE:ethyl acetate=3:1) to give the title compound (2S,4R)-1-(tert-butoxycarbonyl)-4-phenylpyrrolidine-2-carboxylic acid (100 mg, yield 47%) as a colorless oil. LCMS (ESI) m/z=292(M+H)⁺.

### Step 5: 2-benzyl 1-(tert-butyl) (2S,4R)-4-phenylpyrrolidine-1,2-dicarboxylate

To a mixture of (2S,4R)-1-(tert-butoxycarbonyl)-4-phenylpyrrolidine-2-carboxylic acid (100 mg, 0.34 mmol) and Cs₂CO₃ (331 mg, 1.02 mmol) in DMF (6 ml) was added benzyl bromide (64 mg, 0.37 mmol) under N₂ atmosphere at room temperature and the reaction mixture was stirred overnight. The mixture was diluted with water and extracted with ethyl acetate twice. The combined organic layers were washed with brine, dried over anhydrous Na₂SO₄, filtered and concentrated to dryness under reduced pressure. The residue was purified by flash column chromatography on silica gel (PE:Ethyl Acetate=3:1) to give 2-benzyl 1-(tert-butyl) (2S,4R)-4-phenylpyrrolidine-1,2-dicarboxylate (110 mg, yield 85%) as a white solid. LCMS (ESI) m/z=382(M+H)⁺.

### Step 6: benzyl (2S,4R)-4-phenylpyrrolidine-2-carboxylate

To a mixture of 2-benzyl 1-(tert-butyl) (2S,4R)-4-phenylpyrrolidine-1,2-dicarboxylate (110 mg, 0.28 mmol) in HCl/1,4-dioxane (3 mL) was stirred under N₂ atmosphere at room temperature for 2 hours. The reaction mixture was concentrated to dryness under reduced pressure, diluted with DCM and dried under vacuum to give benzyl (2S,4R)-4-phenylpyrrolidine-2-carboxylate (100 mg, crude) as a yellow solid which was used directly in the next step without further purification. LC/MS (ESI) m/z: 282(M+H)⁺.

### Step 7: benzyl (2S,4R)-1-((4-phenoxybutanoyl)glycyl)-4-phenylpyrrolidine-2-carboxylate

To a mixture of benzyl (2S,4R)-4-phenylpyrrolidine-2-carboxylate (100 mg, 0.35 mmol) and (4-phenoxybutanoyl)glycine (78 mg, 0.32 mmol) in DMF (4 mL) was added DIPEA (123 mg, 1.98 mmol) and T₃P (1.22 g, 1.92 mmol, 50% in ethyl acetate) at 0° C. and the mixture was stirred under N₂ atmosphere at room temperature for 2 hours. The organic layer was diluted with water extracted with ethyl acetate twice and the combined organic layers were washed with brine, dried over anhydrous Na₂SO₄, filtered and concentrated to dryness under reduced pressure. The residue was purified by flash chromatography on silica gel (petroleum ether:ethyl acetate=1:1) to give benzyl (2S,4R)-1-((4-phenoxybutanoyl)glycyl)-4-phenylpyrrolidine-2-carboxylate (70 mg, yield 43%) as white solid. LC/MS (ESI) (m/z): 501(M+H)⁺.

### Step 8: (2S,4R)-1-((4-phenoxybutanoyl)glycyl)-4-phenylpyrrolidine-2-carboxylic acid

To a solution of benzyl (2S,4R)-1-((4-phenoxybutanoyl)glycyl)-4-phenylpyrrolidine-2-carboxylate (70 mg, 0.14 mmol) in MeOH (4 mL) was added Pd/C (10 mg, 10% wt) at room temperature and the mixture was stirred under a H₂ balloon for 2 hours. The mixture was filtered and the filtrate was concentrated to dryness under reduced pressure to give (2S,4R)-1-((4-phenoxybutanoyl)glycyl)-4-phenylpyrrolidine-2-carboxylic acid (50 mg, yield 87%) as a colorless oil, which was used directly in the next step without further purification. LC/MS (ESI) (m/z): 411 (M+H)⁺.

### Step 9: (2S,4R)-N-((4-carbamimidoylthiophen-2-yl)methyl)-1-((4-phenoxybutanoyl)glycyl)-4-phenylpyrrolidine-2-carboxamide (Compound 157)

To a mixture of (2S,4R)-1-((4-phenoxybutanoyl)glycyl)-4-phenylpyrrolidine-2-carboxylic acid (50 mg, 0.12 mmol) and 5-(aminomethyl)thiophene-3-carboximidamide (18 mg, 0.12 mmol) in DMF (1 mL) was added DIPEA (46 mg, 0.36 mmol) and PyBop (124 mg, 0.24 mmol) at 0° C. and the mixture was stirred under N₂ atmosphere at room temperature for 2 hours. The organic layer was diluted with water, extracted with ethyl acetate twice and the combined organic layers were washed with brine, dried over anhydrous Na₂SO₄, filtered and concentrated to dryness under reduced pressure. The residue was purified by prep-HPLC to afford Compound 157 (13.3 mg, yield 20.2%) as a white solid. ¹H NMR (400 MHz, CD₃OD) δ 8.54 (s, 1H), 8.22 (dd, J=3.4, 1.6 Hz, 1H), 7.50-7.42 (m, 1H), 7.36-7.27 (m, 4H), 7.24 (dd, J=15.1, 7.5 Hz, 3H), 6.92-6.84 (m, 3H), 4.68-4.56 (m, 2H), 4.54-4.44 (m, 1H), 4.19-4.04 (m, 2H), 4.03-3.89 (m, 3H), 3.61 (t, J=10.3 Hz, 1H), 3.55-3.38 (m, 1H), 2.72-2.60 (m, 1H), 2.47 (t, J=7.3 Hz, 2H), 2.13-1.97 (m, 3H), LC/MS (ESI) (m/z): 548

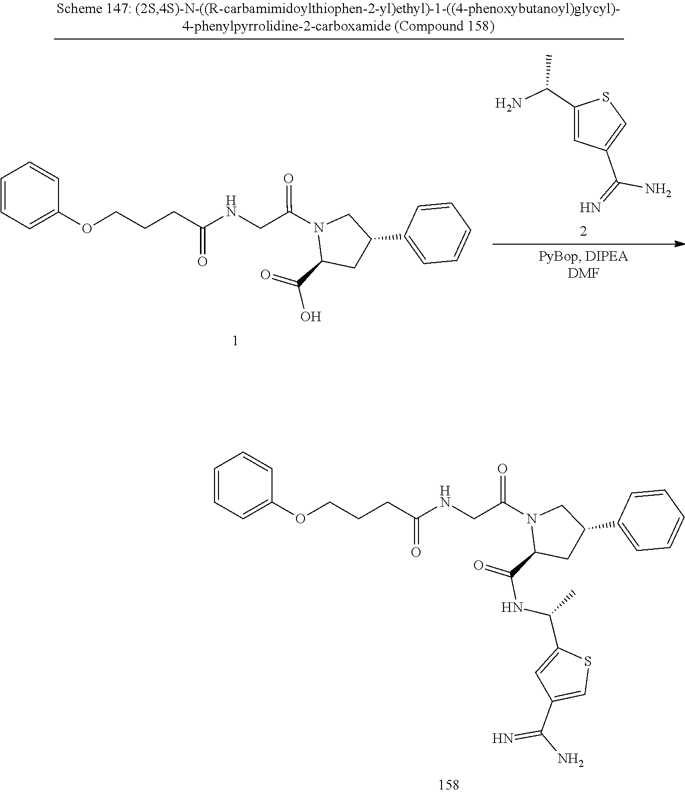 [SMILES: O=C(CCCOc1ccccc1)NCC(=O)N1C[C@H](c2ccccc2)C[C@H]1C(=O)O ; C[C@@H](N)c1cc(C(=N)N)cs1 ; C[C@@H](NC(=O)[C@@H]1C[C@@H](c2ccccc2)CN1C(=O)CNC(=O)CCCOc1ccccc1)c1cc(C(=N)N)cs1]

### Step 1: (2S,4S)-N-((4-carbamimidoylthiophen-2-yl)methyl)-1-((4-phenoxybutanoyl)glycyl)-4-phenylpyrrolidine-2-carboxamide (Compound 158)

To a mixture of (2S,4S)-1-((4-phenoxybutanoyl)glycyl)-4-phenylpyrrolidine-2-carboxylic acid (40 mg, 0.10 mmol) and (R)-5-(1-aminoethyl)thiophene-3-carboximidamide (16 mg, 0.10 mmol) in DMF (1 mL) was added DIPEA (40 mg, 0.31 mmol) and PyBop (100 mg, 0.20 mmol) at 0° C. and the mixture was stirred under N₂ atmosphere at room temperature for 2 hours. The organic layer was diluted with water, extracted with ethyl acetate twice and the combined organic layers were washed with brine, dried over anhydrous Na₂SO₄, filtered and concentrated to dryness under reduced pressure. The residue was purified by prep-HPLC to afford (2S,4S)-N-((R)-1-(4-carbamimidoylthiophen-2-yl)ethyl)-1-((4-phenoxybutanoyl)glycyl)-4-phenylpyrrolidine-2-carboxamide (11.5 mg, yield 19.5%) as a white solid. ¹H NMR (400 MHz, CD₃OD) δ 8.55 (s, 1H), 8.24 (d, J=1.6 Hz, 1H), 7.51 (s, 1H), 7.35-7.28 (m, 4H), 7.24 (dd, J=14.9, 6.9 Hz, 3H), 6.88 (dd, J=7.8, 3.6 Hz, 3H), 5.31 (dt, J=13.4, 6.9 Hz, 1H), 4.65 (dd, J=8.1, 2.7 Hz, 1H), 4.10 (dd, J=9.8, 4.9 Hz, 1H), 4.05 (s, 2H), 3.99 (d, J=6.2 Hz, 2H), 3.71-3.56 (m, 2H), 2.47 (dd, J=9.2, 5.6 Hz, 2H), 2.37 (dd, J=15.0, 7.0 Hz, 2H), 2.07 (dd, J=13.9, 7.0 Hz, 2H), 1.60 (t, J=13.0 Hz, 3H), LC/MS (ESI) (m/z): 562

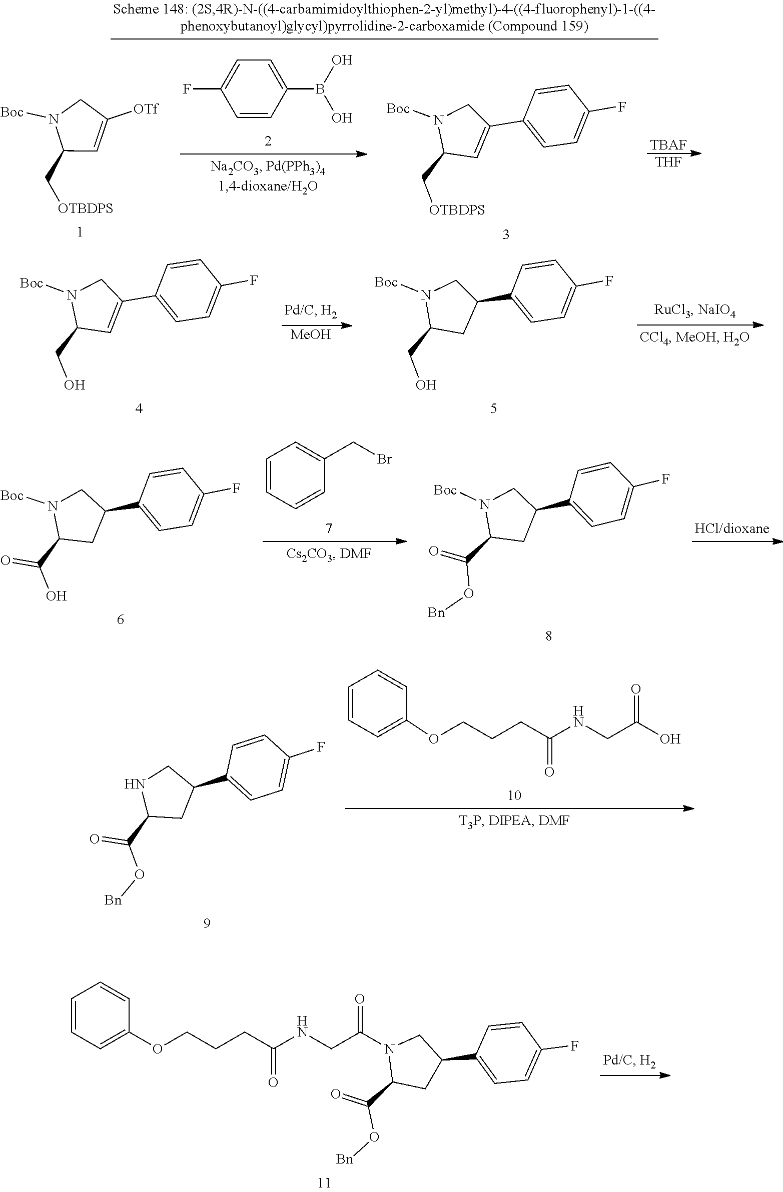

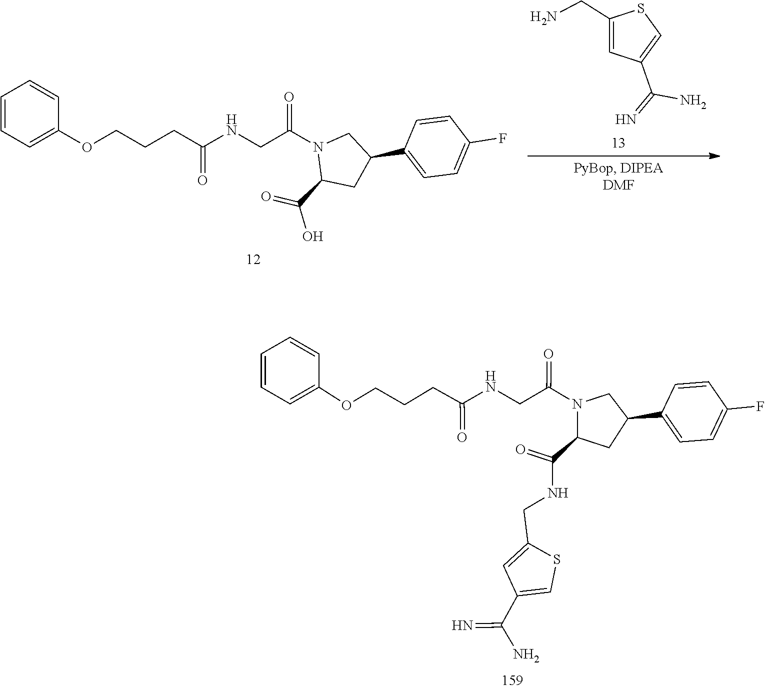 [SMILES: O=C(CCCOc1ccccc1)NCC(=O)N1C[C@@H](c2ccc(F)cc2)C[C@H]1C(=O)O ; N=C(N)c1csc(CN)c1 ; N=C(N)c1csc(CNC(=O)[C@@H]2C[C@H](c3ccc(F)cc3)CN2C(=O)CNC(=O)CCCOc2ccccc2)c1]

### Step 1: tert-butyl (S)-2-(((tert-butyldiphenylsilyl)oxy)methyl)-4-(4-fluorophenyl)-2,5-dihydro-1H-pyrrole-1-carboxylate

To a mixture of tert-butyl (S)-2-(((tert-butyldiphenylsilyl)oxy)methyl)-4-(((trifluoromethyl)sulfonyl)oxy)-2,5-dihydro-1H-pyrrole-1-carboxylate (1.0 g, 1.7 mmol)) and (4-fluorophenyl)boronic acid (280 mg, 2.0 mmol) in 1,4-dioxane (10 ml) and water (3 ml) was added Na₂CO₃ (540 mg, 5.1 mmol) and Pd(PPh₃)₄ (200 mg, 0.17 mmol) under N₂ atmosphere and the mixture was stirred at 80° C. overnight. The mixture was diluted with water and extracted with ethyl acetate twice. The combined organic layers were washed with brine, dried over anhydrous Na₂SO₄, filtered and concentrated to dryness under reduced pressure. The residue was purified by column chromatography on silica gel (PE:EA=10:1) to give tert-butyl (S)-2-(((tert-butyldiphenylsilyl)oxy)methyl)-4-(4-fluorophenyl)-2,5-dihydro-1H-pyrrole-1-carboxylate (900 mg, yield 99.1%) as a white solid. LCMS (ESI) m/z=532 (M+H)⁺.

### Step 2: tert-butyl (S)-4-(4-fluorophenyl)-2-(hydroxymethyl)-2,5-dihydro-1H-pyrrole-1-carboxylate

To a solution of tert-butyl (S)-2-(((tert-butyldiphenylsilyl)oxy)methyl)-4-(4-fluorophenyl)-2,5-dihydro-1H-pyrrole-1-carboxylate (900 mg, 1.69 mmol) in THE (3 mL) was added TBAF (1.32 g, 5.07 mmol) at 0° C. and the mixture was stirred at room temperature for 2 hours. The mixture was diluted with saturated aq.NH₄Cl solution and extracted with ethyl acetate twice. The combined organic layers were washed with brine, dried over anhydrous Na₂SO₄, filtered and concentrated to dryness under reduced pressure. The residue was purified by flash column chromatography on silica gel (PE:ethyl acetate=10:1) to give tert-butyl (S)-4-(4-fluorophenyl)-2-(hydroxymethyl)-2,5-dihydro-1H-pyrrole-1-carboxylate (500 mg, yield 100%) as a white solid. LCMS (ESI) m/z=294(M+H)⁺.

### Step 3: tert-butyl (2S,4R)-4-(4-fluorophenyl)-2-(hydroxymethyl)pyrrolidine-1-carboxylate

To a solution of tert-butyl (S)-4-(4-fluorophenyl)-2-(hydroxymethyl)-2,5-dihydro-1H-pyrrole-1-carboxylate (250 mg, 0.85 mmol) in MeOH (5 mL) was added Pd/C (25 mg, 10% wt) at room temperature and the mixture was stirred under a H₂ balloon at room temperature for 2 hours. The mixture was filtered and the filtrate was concentrated to dryness under reduced pressure to give tert-butyl (2S,4R)-4-(4-fluorophenyl)-2-(hydroxymethyl)pyrrolidine-1-carboxylate (200 mg, yield 79.4%) as colorless oil, which was used directly in the next step without further purification. LC/MS (ESI) (m/z): 296(M+H)⁺.

### Step 4: (2S,4R)-1-(tert-butoxycarbonyl)-4-(4-fluorophenyl)pyrrolidine-2-carboxylic acid

To a mixture of tert-butyl (2S,4R)-4-(4-fluorophenyl)-2-(hydroxymethyl)pyrrolidine-1-carboxylate (200 mg, 0.67 mmol) and NaIO₄ (440 mg, 2.06 mmol) in CCl₄ (0.8 ml)/CH₃CN (0.8 ml)/water (1.2 ml) was stirred under N₂ atmosphere at room temperature for 20 minutes before RuCl₃ (8 mg, 0.041 mmol) was added. The mixture was stirred for 4 hours before diluted with water and extracted with ethyl acetate twice. The combined organic layers were washed with brine, dried over anhydrous Na₂SO₄, filtered and concentrated to dryness under reduced pressure. The residue was purified by flash column chromatography on silica gel (PE:ethyl acetate=3:1) to give the title compound (2S,4R)-1-(tert-butoxycarbonyl)-4-(4-fluorophenyl)pyrrolidine-2-carboxylic acid (100 mg, yield 48%) as a colorless oil. LCMS (ESI) m/z=310(M+H)⁺.

### Step 5: 2-benzyl 1-(tert-butyl) (2S,4R)-4-(4-fluorophenyl)pyrrolidine-1,2-dicarboxylate

To a mixture of (2S,4R)-1-(tert-butoxycarbonyl)-4-(4-fluorophenyl)pyrrolidine-2-carboxylic acid (100 mg, 0.31 mmol) and Cs₂CO₃ (90 mg, 0.93 mmol) in DMF (6 ml) was added benzyl bromide (58 mg, 0.35 mmol) under N₂ atmosphere at room temperature and the reaction mixture was stirred overnight. The mixture was diluted with water and extracted with ethyl acetate twice. The combined organic layers were washed with brine, dried over anhydrous Na₂SO₄, filtered and concentrated to dryness under reduced pressure. The residue was purified by flash column chromatography on silica gel (PE:Ethyl Acetate=3:1) to give 2-benzyl 1-(tert-butyl) (2S,4R)-4-(4-fluorophenyl)pyrrolidine-1,2-dicarboxylate (110 mg, yield 89%) as a white solid. LCMS (ESI) m/z=399 (M+H)⁺.

### Step 6: benzyl (2S,4R)-4-(4-fluorophenyl)pyrrolidine-2-carboxylate

To a solution of 2-benzyl 1-(tert-butyl) (2S,4R)-4-(4-fluorophenyl)pyrrolidine-1,2-dicarboxylate (110 mg, 0.26 mmol) in HCl/1,4-dioxane (3 mL) was stirred under N₂ atmosphere at room temperature for 2 hours. The reaction mixture was concentrated to dryness under reduced pressure, diluted with DCM and dried under vacuum to give benzyl (2S,4R)-4-(4-fluorophenyl)pyrrolidine-2-carboxylate (100 mg, crude) as a yellow solid which was used directly in the next step without further purification. LC/MS (ESI) m/z: 299 (M+H)⁺.

### Step 7: benzyl (2S,4R)-4-(4-fluorophenyl)-1-((4-phenoxybutanoyl)glycyl)pyrrolidine-2-carboxylate

To a mixture of benzyl (2S,4R)-4-(4-fluorophenyl)pyrrolidine-2-carboxylate (100 mg, 0.32 mmol) and (4-phenoxybutanoyl)glycine (78 mg, 0.32 mmol) in DMF (4 mL) was added DIPEA (255 mg, 1.98 mmol) and T₃P (620 mg, 50% wt in EA, 0.99 mmol) at 0° C. and the mixture was stirred under N₂ atmosphere at room temperature for 2 hours. The organic layer was diluted with water, extracted with ethyl acetate twice and the combined organic layers were washed with brine, dried over anhydrous Na₂SO₄, filtered and concentrated to dryness under reduced pressure. The residue was purified by flash chromatography on silica gel (petroleum ether:ethyl acetate=1:1) to give benzyl (2S,4R)-4-(4-fluorophenyl)-1-((4-phenoxybutanoyl)glycyl) pyrrolidine-2-carboxylate (70 mg, yield 42%) as white solid. LC/MS (ESI) (m/z): 519(M+H)⁺.

### Step 8: (2S,4R)-4-(4-fluorophenyl)-1-((4-phenoxybutanoyl)glycyl)pyrrolidine-2-carboxylic acid

To a solution of benzyl (2S,4R)-4-(4-fluorophenyl)-1-((4-phenoxybutanoyl)glycyl)pyrrolidine-2-carboxylate (70 mg, 0.12 mmol) in MeOH (4 mL) was added Pd/C (10 mg, 10% wt) at room temperature and the mixture was stirred under a H₂ balloon for 2 hours. The mixture was filtered and the filtrate was concentrated to dryness under reduced pressure to give (2S,4R)-4-(4-fluorophenyl)-1-((4-phenoxybutanoyl)glycyl)pyrrolidine-2-carboxylic acid (30 mg, yield 58%) as a colorless oil, which was used directly in the next step without further purification. LC/MS (ESI) (m/z): 429 (M+H)⁺.

### Step 9: (2S,4R)-N-((4-carbamimidoylthiophen-2-yl)methyl)-4-(4-fluorophenyl)-1-((4-phenoxybutanoyl)glycyl)pyrrolidine-2-carboxamide (Compound 159)

To a mixture of (2S,4R)-4-(4-fluorophenyl)-1-((4-phenoxybutanoyl)glycyl)pyrrolidine-2-carboxylic acid (30 mg, 0.07 mmol) and 5-(aminomethyl)thiophene-3-carboximidamide (11 mg, 0.07 mmol) in DMF (1 mL) was added DIPEA (54 mg, 0.42 mmol) and PyBop (218 mg, 0.21 mmol) at 0° C. and the mixture was stirred under N₂ atmosphere at room temperature for 2 hours. The organic layer was diluted with water, extracted with ethyl acetate twice and the combined organic layers were washed with brine, dried over anhydrous Na₂SO₄, filtered and concentrated to dryness under reduced pressure. The residue was purified by prep-HPLC to afford Compound 159 (5.8 mg, yield 14.6%) as a white solid. ¹H NMR (400 MHz, CD₃OD) δ 8.55 (s, 1H), 8.22 (d, J=1.5 Hz, 1H), 7.45 (s, 1H), 7.34 (dt, J=8.6, 4.3 Hz, 2H), 7.27-7.21 (m, 2H), 7.06 (dd, J=11.9, 5.6 Hz, 2H), 6.92-6.87 (m, 3H), 4.58 (d, J=3.3 Hz, 2H), 4.56-4.46 (m, 1H), 4.27-4.03 (m, 2H), 3.99 (dd, J=11.7, 5.4 Hz, 3H), 3.61-3.47 (m, 2H), 2.74-2.60 (m, 1H), 2.47 (t, J=7.3 Hz, 2H), 2.08 (t, J=7.1 Hz, 2H), 2.01 (dd, J=20.3, 9.8 Hz, 1H), LC/MS (ESI) (m/z): 566 (M+H)⁺.

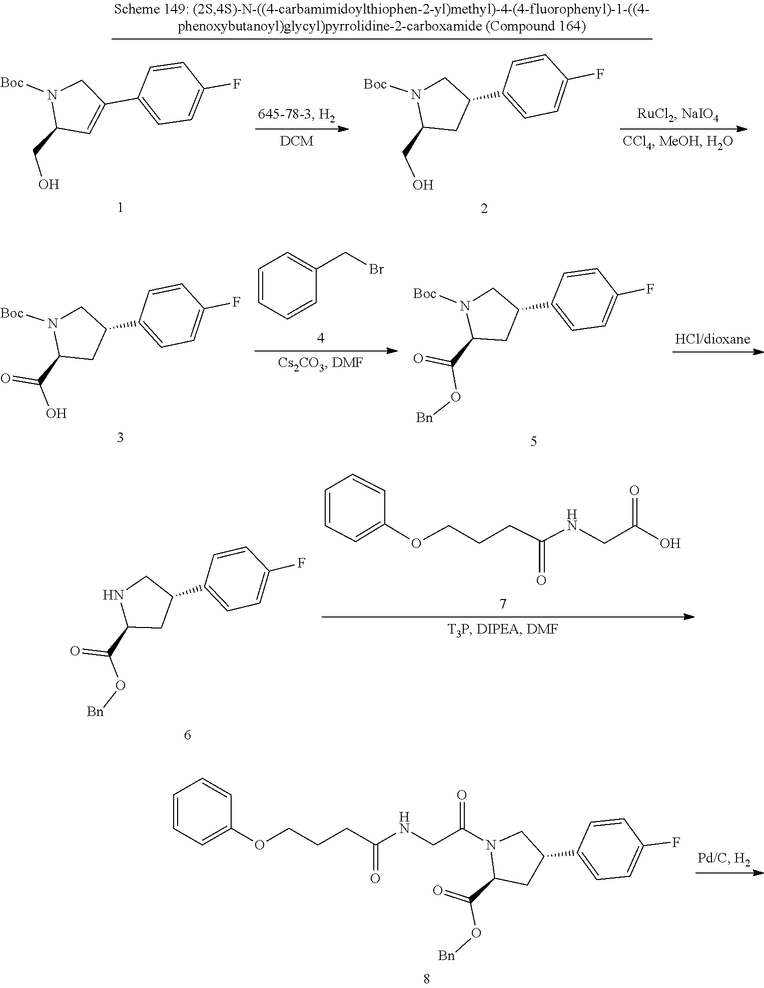

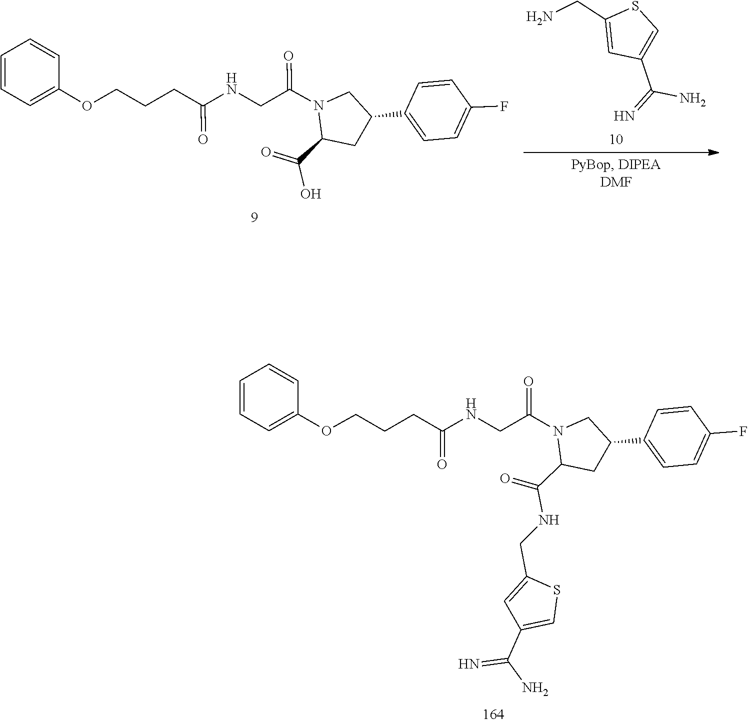 [SMILES: O=C(CCCOc1ccccc1)NCC(=O)N1C[C@H](c2ccc(F)cc2)C[C@H]1C(=O)O ; N=C(N)c1csc(CN)c1 ; N=C(N)c1csc(CNC(=O)C2C[C@@H](c3ccc(F)cc3)CN2C(=O)CNC(=O)CCCOc2ccccc2)c1]

### Step 1: tert-butyl (2S,4S)-4-(4-fluorophenyl)-2-(hydroxymethyl)pyrrolidine-1-carboxylate

To a solution of tert-butyl (S)-4-(4-fluorophenyl)-2-(hydroxymethyl)-2,5-dihydro-1H-pyrrole-1-carboxylate (250 mg, 0.85 mmol) in DCM (5 mL) was added (1,5-cyclooctadiene) (pyridine)(tricyclohexylphosphine)iridium(I) hexafluorophosphate (25 mg, 0.03 mmol) and the mixture was stirred under a H₂ balloon at room temperature for 12 hours. The mixture was filtered and the filtrate was concentrated to dryness under reduced pressure to give tert-butyl (2S,4S)-4-(4-fluorophenyl)-2-(hydroxymethyl)pyrrolidine-1-carboxylate (200 mg, yield 79.4%) as white solid, which was used directly in the next step. LC/MS (ESI) (m/z): 296(M+H)⁺.

### Step 2: (2S,4S)-1-(tert-butoxycarbonyl)-4-(4-fluorophenyl)pyrrolidine-2-carboxylic acid

To a mixture of tert-butyl (2S,4S)-4-(4-fluorophenyl)-2-(hydroxymethyl)pyrrolidine-1-carboxylate (200 mg, 0.67 mmol) and NaIO₄ (440 mg, 2.06 mmol) in CCl₄ (0.8 ml)/CH₃CN (0.8 ml)/water (1.2 ml) was stirred under N₂ atmosphere at room temperature for 20 minutes. The reaction mixture was added RuCl₃ (8 mg, 0.041 mmol) and stirred for 4 hours. The mixture was diluted with water and extracted with ethyl acetate twice. The combined organic layers were washed with brine, dried over anhydrous Na₂SO₄, filtered and concentrated to dryness under reduced pressure. The residue was purified by flash column chromatography on silica gel (PE:Ethyl Acetate=3:1) to give the title compound (2S,4S)-1-(tert-butoxycarbonyl)-4-(4-fluorophenyl)pyrrolidine-2-carboxylic acid (60 mg, yield 29.0%) as colorless oil. LCMS (ESI) m/z=310 (M+H)⁺.

### Step 3: 2-benzyl 1-(tert-butyl) (2S,4S)-4-(4-fluorophenyl)pyrrolidine-1,2-dicarboxylate

To a mixture of (2S,4S)-1-(tert-butoxycarbonyl)-4-(4-fluorophenyl)pyrrolidine-2-carboxylic acid (60 mg, 0.19 mmol) and Cs₂CO₃ (189 mg, 0.58 mmol) in DMF (2 ml) was added benzyl bromide (36 mg, 0.21 mmol) and the reaction mixture was stirred under N₂ atmosphere at room temperature overnight. The mixture was diluted with water and extracted with ethyl acetate twice. The combined organic layers were washed with brine, dried over anhydrous Na₂SO₄, filtered and concentrated to dryness under reduced pressure. The residue was purified by flash column chromatography on silica gel (PE:Ethyl Acetate=3:1) to give 2-benzyl 1-(tert-butyl) (2S,4S)-4-(4-fluorophenyl)pyrrolidine-1,2-dicarboxylate (70 mg, yield 90.3%) as white solid. LCMS (ESI) m/z=399 (M+H)⁺.

### Step 4: benzyl (2S,4S)-4-(4-fluorophenyl)pyrrolidine-2-carboxylate

To a solution of 2-benzyl 1-(tert-butyl) (2S,4S)-4-(4-fluorophenyl)pyrrolidine-1,2-dicarboxylate (70 mg, 0.17 mmol) in HCl/1,4-dioxane (2 mL) was stirred under N₂ atmosphere at room temperature for 2 hours. The reaction mixture was concentrated to dryness under reduced pressure, diluted with DCM again and dried under vacuum to give benzyl (2S,4S)-4-(4-fluorophenyl)pyrrolidine-2-carboxylate (70 mg, quantitative) as yellow solid which was used directly in the next step without further purification. LC/MS (ESI) m/z: 299 (M+H)⁺.

### Step 5: benzyl (2S,4S)-4-(4-fluorophenyl)-1-((4-phenoxybutanoyl)glycyl)pyrrolidine-2-carboxylate

To a mixture of benzyl (2S,4S)-4-(4-fluorophenyl)pyrrolidine-2-carboxylate (70 mg, 0.23 mmol) and (4-phenoxybutanoyl)glycine (55 mg, 0.23 mmol) in DMF (3 mL) was added DIPEA (180 mg, 1.40 mmol) and T₃P (440 mg, 0.70 mmol, 50% in ethyl acetate) at 0° C. and the mixture was stirred under N₂ atmosphere at room temperature for 2 hours. The organic layer was diluted with water extracted with ethyl acetate twice and the combined organic layers were washed with brine, dried over anhydrous Na₂SO₄, filtered and concentrated to dryness under reduced pressure. The residue was purified by flash chromatography on silica gel (petroleum ether:ethyl acetate=1:1) to give benzyl (2S,4S)-4-(4-fluorophenyl)-1-((4-phenoxybutanoyl)glycyl) pyrrolidine-2-carboxylate (40 mg, yield 33%) as white solid. LC/MS (ESI) (m/z): 519(M+H)⁺.

### Step 6: (2S,4S)-4-(4-fluorophenyl)-1-((4-phenoxybutanoyl)glycyl)pyrrolidine-2-carboxylic acid

To a solution of benzyl (2S,4S)-4-(4-fluorophenyl)-1-((4-phenoxybutanoyl)glycyl)pyrrolidine-2-carboxylate (40 mg, 0.07 mmol) in MeOH (3 mL) was added Pd/C (10 mg, 10% wt.) at room temperature and the mixture was stirred under a H₂ balloon for 2 hours. The mixture was filtered and the filtrate was concentrated to dryness under reduced pressure to give (2S,4S)-4-(4-fluorophenyl)-1-((4-phenoxybutanoyl)glycyl)pyrrolidine-2-carboxylic acid (20 mg, yield 60%) as colorless oil, which was used directly in the next step without further purification. LC/MS (ESI) (m/z): 429 (M+H)⁺.

### Step 7: (2S,4S)-N-((4-carbamimidoylthiophen-2-yl)methyl)-4-(4-fluorophenyl)-1-((4-phenoxybutanoyl)glycyl)pyrrolidine-2-carboxamide (Compound 164)

To a mixture of (2S,4S)-4-(4-fluorophenyl)-1-((4-phenoxybutanoyl)glycyl)pyrrolidine-2-carboxylic acid (20 mg, 0.04 mmol) and 5-(aminomethyl)thiophene-3-carboximidamide (7 mg, 0.04 mmol) in DMF (1 mL) was added DIPEA (130 mg, 0.14 mmol) and PyBop (48 mg, 0.09 mmol) at 0° C. and the mixture was stirred under N₂ atmosphere at room temperature for 2 hours. The organic layer was diluted with water, extracted with ethyl acetate twice and the combined organic layers were washed with brine, dried over anhydrous Na₂SO₄, filtered and concentrated to dryness under reduced pressure. The residue was purified by prep-HPLC to afford Compound 164 (1.5 mg, yield 5.76%) as white solid. ¹H NMR (400 MHz, CD₃OD) δ 8.54 (s, 2H), 8.23 (d, J=1.6 Hz, 1H), 7.46 (d, J=27.4 Hz, 1H), 7.34 (dd, J=8.8, 5.4 Hz, 2H), 7.27-7.19 (m, 2H), 7.07 (t, J=8.8 Hz, 2H), 6.94-6.84 (m, 3H), 4.69-4.64 (m, 2H), 4.15-4.08 (m, 2H), 4.01 (dd, J=10.5, 4.1 Hz, 3H), 3.60 (dd, J=20.2, 12.0 Hz, 2H), 2.48 (t, J=7.4 Hz, 2H), 2.36 (d, J=7.9 Hz, 2H), 2.13-2.04 (m, 3H).LCMS (ESI) m/z=566(M+H)⁺.

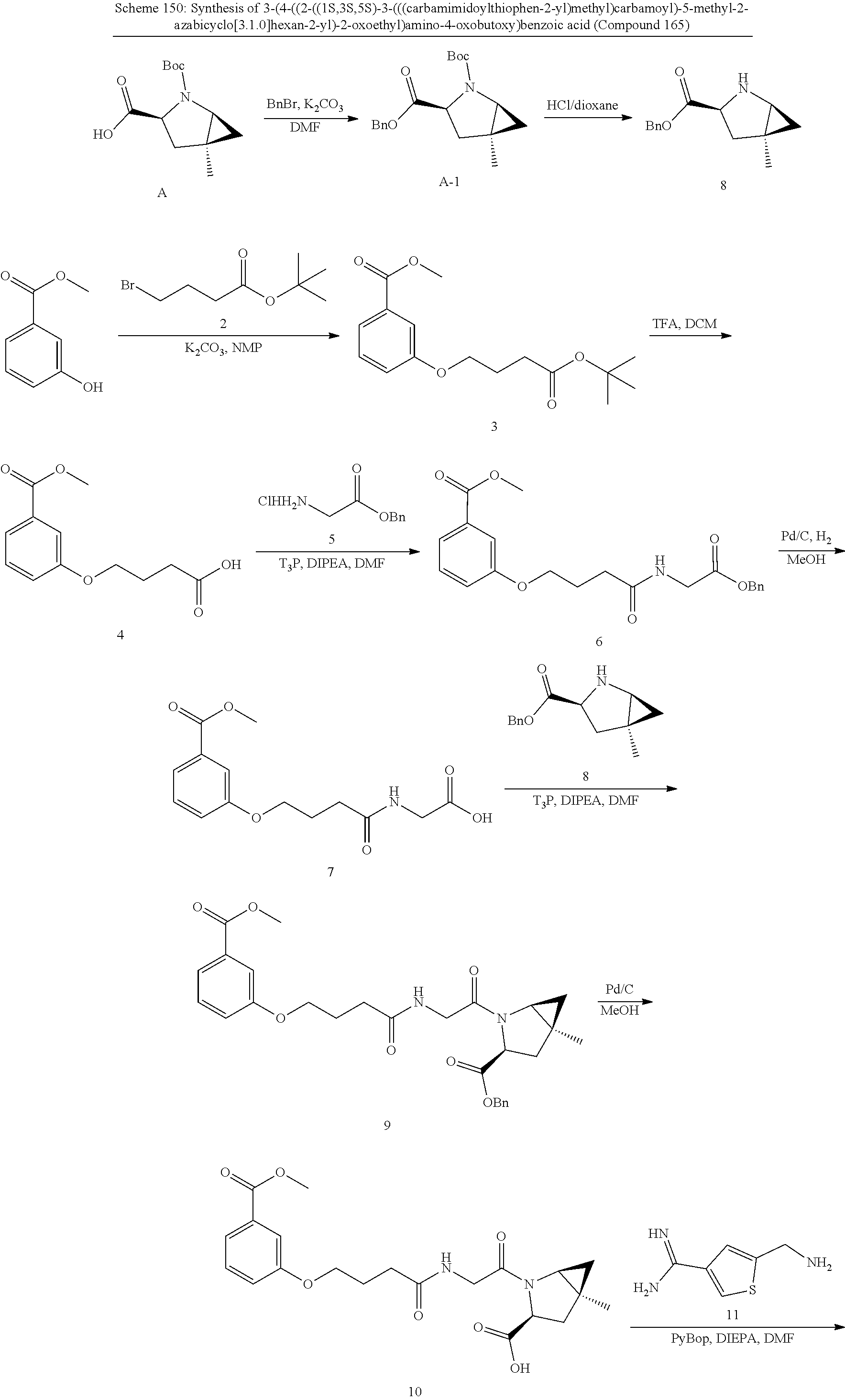

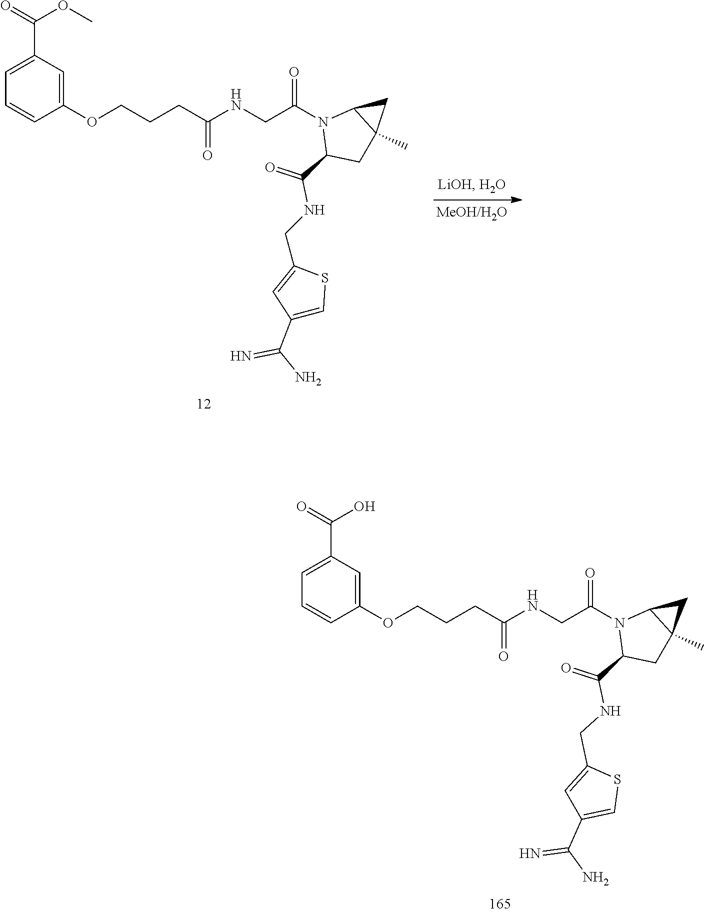 [SMILES: COC(=O)c1cccc(OCCCC(=O)NCC(=O)N2[C@H]3C[C@@]3(C)C[C@H]2C(=O)NCc2cc(C(=N)N)cs2)c1 ; C[C@@]12C[C@@H]1N(C(=O)CNC(=O)CCCOc1cccc(C(=O)O)c1)[C@H](C(=O)NCc1cc(C(=N)N)cs1)C2]

### Step 1: methyl 3-(4-(tert-butoxy)-4-oxobutoxy)benzoate (2)

To a solution of methyl 3-hydroxybenzoate (609 mg, 4.0 mmol), tert-butyl 4-bromobutanoate (4.5 g, 20.0 mmol) in NMP (7 mL) was added K₂CO₃ (3.3 g, 24.0 mmol), and the reaction mixture was stirred under N₂ atmosphere at 120° C. overnight, diluted with 60 mL water and the mixture was extracted with EtOAc (40 ml×2). The combined organic layers washed with brine, dried over anhydrous Na₂SO₄ and concentrated to dryness under reduced pressure. The residue was purified by flash column (PE:EtOAc=10:1) to give the methyl 3-(4-(tert-butoxy)-4-oxobutoxy)benzoate (1.1 g, yield 93.7%) as yellow oil.

### Step 2: 4-(3-(methoxycarbonyl)phenoxy)butanoic acid (4)

To a solution of methyl 3-(4-(tert-butoxy)-4-oxobutoxy)benzoate (1.1 g, 3.74 mmol) in DCM (20 mL) and TFA (11 mL) was stirred under N₂ atmosphere at room temperature for 1 hour. The mixture was concentrated to dryness under reduced pressure. The residue was purified by flash column (PE:EtOAc=5:1) to give 4-(3-(methoxycarbonyl)phenoxy)butanoic acid (850 mg, yield 95.1%) as yellow oil. LC/MS(ESI) m/z: 239 (M+H)⁺.

### Step 3: methyl 3-(4-((2-(benzyloxy)-2-oxoethyl)amino)-4-oxobutoxy)benzoate (6)

To a mixture of 4-(3-(methoxycarbonyl)phenoxy)butanoic acid (850 mg, 3.57 mmol), benzyl glycinate hydrochloride (860 mg, 4.28 mmol) in DMF (8 mL) was added DIPEA (2770 mg, 21.42 mmol) and T₃P (6.8 g, 10.7 mmol, 50% in EtOAc) at 0° C. and the mixture was stirred under N₂ atmosphere at room temperature for 1 hour. The mixture was diluted with ethyl acetate and washed with saturated 1N aq.NaHCO₃ solution. The aqueous layer was extracted with EtOAc twice and the combined organic layers were washed with brine, dried over anhydrous Na₂SO₄, filtered and concentrated to dryness under reduced pressure. The residue was purified by flash chromatography on silica gel (PE:EtOAc=5:1) to give methyl 3-(4-((2-(benzyloxy)-2-oxoethyl)amino)-4-oxobutoxy)benzoate (800 mg, yield 58.1%) as yellow oil. LC/MS (ESI) (m/z): 386(M+H)⁺.

### Step 4: methyl 3-(4-((2-(benzyloxy)-2-oxoethyl)amino)-4-oxobutoxy)benzoate (7)

To a solution of methyl 3-(4-((2-(benzyloxy)-2-oxoethyl)amino)-4-oxobutoxy)benzoate (800 mg, 2.08 mmol) in MeOH (10 mL) was added Pd/C (80 mg, 10% wt.) and the reaction was stirred under a H₂ balloon at room temperature for 3 hours. The mixture was filtered and the filtrate was concentrated to dryness under reduced pressure to give methyl 3-(4-((2-(benzyloxy)-2-oxoethyl)amino)-4-oxobutoxy)benzoate (593 mg, yield 96.7%) as white solid, which was used directly in the next step.

LC/MS (ESI) (m/z):296(M+H)⁺.

### Step 5: benzyl (1S,3S,5S)-5-methyl-2-azabicyclo[3.1.0]hexane-3-carboxylate (A-1)

To a mixture of (1 S,3S,5S)-2-(tert-butoxycarbonyl)-5-methyl-2-azabicyclo[3.1.0]hexane-3-carboxylic acid (180 mg, 0.7 mmol) and (bromomethyl)benzene (192 mg, 1.1 mmol) in DMF (2 mL) was added K₂CO₃ (317 mg, 2.3 mmol), and the reaction mixture was stirred at 80° C. for 2 hours, diluted with 20 mL water and the mixture was extracted with EA (40 ml×2). The combined organic layers washed with brine, dried over anhydrous Na₂SO₄ and concentrated to dryness under reduced pressure. The residue was purified by flash column (PE:EtOAc=10:1) to give benzyl (1S,3S,5S)-5-methyl-2-azabicyclo[3.1.0]hexane-3-carboxylate (240 mg, yield 97.1%) as yellow oil. LC/MS(ESI) m/z: 288 (M+H+56)⁺.

### Step 6: benzyl (1S,3S,5S)-5-methyl-2-azabicyclo[3.1.0]hexane-3-carboxylate (8)

To a solution of benzyl (1 S,3S,5S)-5-methyl-2-azabicyclo[3.1.0]hexane-3-carboxylate (240 mg, 0.73 mmol) in MeOH (2 mL) and HCl/1,4-dioxane (3 mL) was stirred under N₂ atmosphere at room temperature for 2 hours. The mixture was concentrated under reduced pressure to give benzyl (1S,3S,5S)-5-methyl-2-azabicyclo[3.1.0]hexane-3-carboxylate (194 mg, yield 99%) as yellow solid. LC/MS(ESI) m/z: 232 (M+H)⁺.

### Step 7: benzyl (1S,3S,5S)-2-((4-(3-(methoxycarbonyl)phenoxy)butanoyl)glycyl)-5-methyl-2-azabicyclo[3.1.0]hexane-3-carboxylate (9)

To a mixture of (4-(3-(methoxycarbonyl)phenoxy)butanoyl)glycine (180 mg, 0.61 mmol) and benzyl (1S,3S,5S)-5-methyl-2-azabicyclo[3.1.0]hexane-3-carboxylate (164 mg, 0.61 mmol) in DMF (2 mL) was added DIPEA (473 mg, 3.66 mmol) and T₃P (1.16 g, 1.83 mmol, 50% in EtOAc) at 0° C. and the mixture was stirred under N₂ atmosphere at room temperature for 1 hour. The mixture was diluted with EtOAc and washed with saturated 1N aq.NaHCO₃ solution. The aqueous layer was extracted with EtOAc twice and the combined organic layers were washed with brine, dried over anhydrous Na₂SO₄, filtered and concentrated to dryness under reduced pressure. The residue was purified by flash column (PE:EtOAc=2:1) to give benzyl (1S,3S,5S)-2-((4-(3-(methoxycarbonyl)phenoxy)butanoyl)glycyl)-5-methyl-2-azabicyclo[3.1.0]hexane-3-carboxylate (250 mg, yield 80.6%) as yellow oil. LC/MS (ESI) (m/z): 509(M+H)⁺.

### Step 8: (1S,3S,5S)-2-((4-(3-(methoxycarbonyl)phenoxy)butanoyl)glycyl)-5-methyl-2-azabicyclo[3.1.0]hexane-3-carboxylic acid (10)

To a solution of benzyl (1S,3S,5S)-2-((4-(3-(methoxycarbonyl)phenoxy)butanoyl)glycyl)-5-methyl-2-azabicyclo[3.1.0]hexane-3-carboxylate (240 mg, 0.47 mmol) in MeOH (5 mL) was added Pd/C (30 mg, 10% wt.) and the reaction was stirred under a H₂ balloon at room temperature for 3 hours. The mixture was filtered and the filtrate was concentrated to dryness under reduced pressure to give (1S,3S,5S)-2-((4-(3-(methoxycarbonyl)phenoxy)butanoyl) glycyl)-5-methyl-2-azabicyclo[3.1.0]hexane-3-carboxylic acid (185 mg, yield 94.1%) as yellow oil, which was used directly in the next step. LC/MS (ESI) (m/z):419(M+H)⁺.

### Step 9: methyl 3-(4-((2-((1S,3S,5S)-3-(((4-carbamimidoylthiophen-2-yl)methyl)carbamoyl)-5-methyl-2-azabicyclo[3.1.0]hexan-2-yl)-2-oxoethyl)amino)-4-oxobutoxy)benzoate (12)

To a mixture of (1S,3S,5S)-2-((4-(3-(methoxycarbonyl)phenoxy)butanoyl)glycyl)-5-methyl-2-azabicyclo[3.1.0]hexane-3-carboxylic acid (100 mg, 0.24 mmol) and 5-(aminomethyl)thiophene-3-carboximidamide (69 mg, 0.36 mmol) in DMF (1 ml), the mixture was added DIPEA (186 mg, 1.44 mmol) and PyBop (187 mg, 0.36 mmol) at 0° C. and the mixture was stirred under N₂ atmosphere at room temperature overnight. The mixture was diluted with EtOAc and washed with saturated aq.NaHCO₃ solution. The aqueous layer was extracted with EtOAc twice and the combined organic layers were washed with brine, dried over anhydrous Na₂SO₄, filtered and concentrated to dryness under reduced pressure. The residue was purified by flash column (DCM:MeOH=15:1) to give methyl 3-(4-((2-((1 S,3S,5S)-3-(((4-carbamimidoylthiophen-2-yl)methyl)carbamoyl)-5-methyl-2-azabicyclo[3.1.0]hexan-2-yl)-2-oxoethyl)amino)-4-oxobutoxy)benzoate (81 mg, yield 61.0%) as white solid. LC/MS (ESI) (m/z): 556(M+H)⁺.

### Step 10: 3-(4-((2-((1S,3S,5S)-3-(((4-carbamimidoylthiophen-2-yl)methyl)carbamoyl)-5-methyl-2-azabicyclo[3.1.0]hexan-2-yl)-2-oxoethyl)amino)-4-oxobutoxy)benzoic acid (Compound 165)

To a solution of methyl 3-(4-((2-((1 S,3S,5S)-3-(((4-carbamimidoylthiophen-2-yl)methyl)carbamoyl)-5-methyl-2-azabicyclo[3.1.0]hexan-2-yl)-2-oxoethyl)amino)-4-oxobutoxy)benzoate (70 mg, 0.13 mmol) in MeOH (1 mL) and water (0.5 mL) was added a solid of LiOH·H₂O (16 mg, 0.38 mmol) at 40° C. and the mixture was stirred at room temperature for 16 hours. The mixture was acidified with 1N aq.HCl solution to pH=3 and concentrated to dryness under reduced pressure and purified by prep-HPLC to give Compound 165 (5.0 mg, yield 7.4%) as a white solid. ¹H NMR (400 MHz, CD₃OD) δ 8.20 (d, J=1.6 Hz, 1H), 7.58 (d, J=7.6 Hz, 1H), 7.52 (s, 1H), 7.36 (dd, J=15.7, 7.6 Hz, 2H), 7.14 (dd, J=8.2, 1.9 Hz, 1H), 4.80-4.77 (m, 1H), 4.55-4.43 (m, 2H), 4.16 (q, J=16.6 Hz, 2H), 4.05 (t, J=6.2 Hz, 2H), 2.47 (t, J=7.4 Hz, 2H), 2.37 (t, J=11.8 Hz, 1H), 2.19-2.12 (m, 1H), 2.11-2.05 (m, 2H), 1.25 (d, J=15.0 Hz, 3H), 1.19-0.96 (m, 2H), 0.79 (t, J=5.8 Hz, 1H). LC/MS (ESI) (m/z): 542(M+H)⁺.

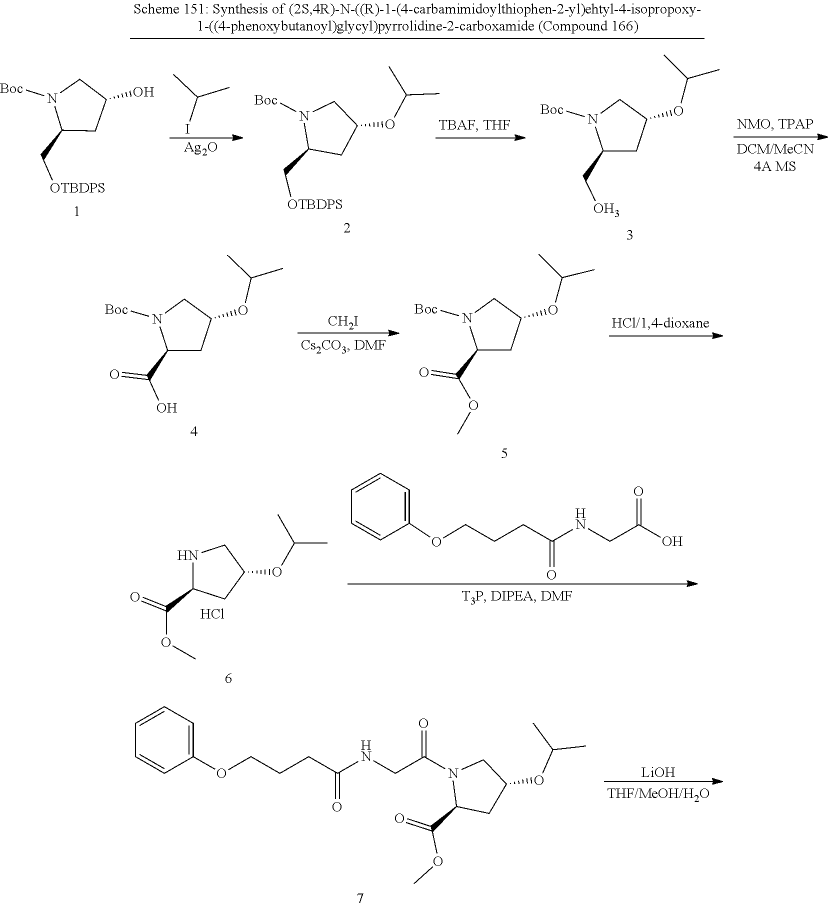

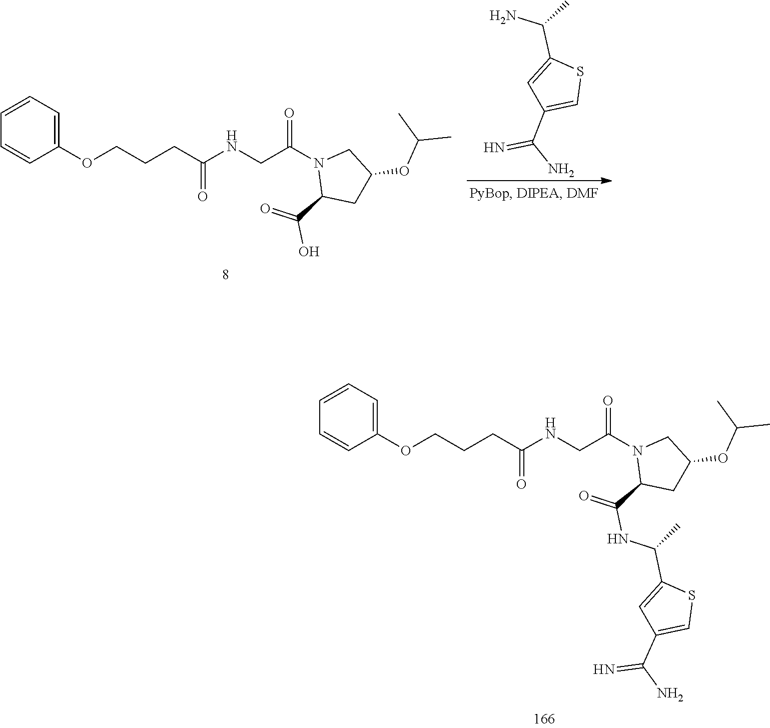 [SMILES: CC(C)O[C@@H]1C[C@@H](C(=O)O)N(C(=O)CNC(=O)CCCOc2ccccc2)C1 ; C[C@@H](N)c1cc(C(=N)N)cs1 ; CC(C)O[C@@H]1C[C@@H](C(=O)N[C@H](C)c2cc(C(=N)N)cs2)N(C(=O)CNC(=O)CCCOc2ccccc2)C1]

### Step 1: tert-butyl (2S,4R)-2-(((tert-butyldiphenylsilyl)oxy)methyl)-4-isopropoxypyrrolidine-1-carboxylate (2)

To a solution of tert-butyl (2S)-2-{[(tert-butyldiphenylsilyl)oxy]methyl}-4-hydroxypyrrolidine-1-carboxylate (3 g, 6.58 mmol) in 2-iodopropane (20 mL) was added silver oxide (4.6 g, 19.75 mmol). The reaction mixture was stirred in a sealed tube at 60° C. for 16 hours. The mixture was diluted with ethyl acetate, filtered and the filtrate was concentrated to dryness under reduced pressure. The residue was purified by flash chromatography on silica gel (PE:ethyl acetate=5:1) to afford tert-butyl (2S)-2-{[(tert-butyldiphenylsilyl)oxy]methyl}-4-isopropoxypyrrolidine-1-carboxylate (1.3 g, yield 39.7%) as white solid. LC/MS (ESI) m/z: 498 (M+H)⁺.

### Step 2: tert-butyl (2S,4R)-2-(hydroxymethyl)-4-isopropoxypyrrolidine-1-carboxylate (3)

To a solution of tert-butyl (2S)-2-{[(tert-butyldiphenylsilyl)oxy]methyl}-4-isopropoxypyrrolidine-1-carboxylate (1.3 g, 2.61 mmol) in THE (20 mL) was added TBAF (2.0 g, 7.84 mmol) under N₂ atmosphere and the reaction mixture was stirred at room temperature overnight. The mixture was diluted with ethyl acetate, washed with saturated aq.NH₄Cl solution and brine, dried over anhydrous Na₂SO₄, filtered and concentrated to dryness under reduced pressure. The residue was purified by flash chromatography on silica gel (PE:ethyl acetate=5:1) to afford tert-butyl (2S)-2-(hydroxymethyl)-4-isopropoxypyrrolidine-1-carboxylate (670 mg, yield 98.9%) as white solid. LC/MS (ESI) m/z:260 (M+H)⁺.

### Step 3: (2S,4R)-1-(tert-butoxycarbonyl)-4-isopropoxypyrrolidine-2-carboxylic acid (4)

To a solution of tert-butyl (2S,4R)-2-(hydroxymethyl)-4-isopropoxypyrrolidine-1-carboxylate (200 mg, 0.77 mmol) in DCM/MeCN (5.5 mL, 10/1 v/v) was added 4 Å molecular sieves at (164 mg) and NMO (181 mg, 1.54 mmol) 0° C. After stirred at 0° C. for 10 minutes, TPAP (27 mg, 0.077 mmol) was added into the above mixture under N₂ atmosphere and the reaction mixture was stirred at 0° C. for 2 hours. The mixture was diluted with ethyl acetate and washed with H₂O. The aqueous layer was extracted with ethyl acetate twice and the combined organic layers were washed with brine, dried over anhydrous Na₂SO₄, filtered and concentrated to dryness under reduced pressure to afford (2S,4R)-1-(tert-butoxycarbonyl)-4-isopropoxypyrrolidine-2-carboxylic acid (120 mg, yield 56.9%) as white solid. LC/MS (ESI) m/z: 274 (M+H)⁺.

### Step 4: 1-(tert-butyl) 2-methyl (2S,4R)-4-isopropoxypyrrolidine-1,2-dicarboxylate (5)

To a solution of (2S,4R)-1-(tert-butoxycarbonyl)-4-isopropoxypyrrolidine-2-carboxylic acid (120 mg, 0.44 mmol) in DMF (3 mL) was added Cs₂CO₃ (157 mg, 0.48 mmol) and CH₃I (75 mg, 0.53 mmol) under N₂ atmosphere and the reaction mixture was stirred at room temperature for 2 hours. The mixture was diluted with ethyl acetate and washed with H₂O. The aqueous layer was extracted with ethyl acetate twice and the combined organic layers were washed with brine, dried over anhydrous Na₂SO₄, filtered and concentrated to dryness under reduced pressure. The residue was purified by flash chromatography on silica gel (PE:ethyl acetate=5:1) to afford 1-tert-butyl 2-methyl (2S,4R)-4-isopropoxypyrrolidine-1,2-dicarboxylate (80 mg, yield 63.4%) as white solid. LC/MS (ESI) m/z: 288 (M+H)⁺.

### Step 5: methyl (2S,4R)-4-isopropoxypyrrolidine-2-carboxylate hydrochloride (6)

To a solution of 1-tert-butyl 2-methyl (2S,4R)-4-isopropoxypyrrolidine-1,2-dicarboxylate (80 mg, 0.28 mmol) in 1,4-dioxane (2 mL) was added HCl/1,4-dioxane (1 mL) under N₂ atmosphere and the reaction mixture was stirred at room temperature for 2 hours. The mixture was concentrated to dryness under reduced pressure to afford methyl (2S,4R)-4-isopropoxypyrrolidine-2-carboxylate hydrochloride (65 mg, yield 100%) as white solid. LC/MS (ESI) m/z:188 (M+H)⁺.

### Step 6: methyl (2S,4R)-4-isopropoxy-1-((4-phenoxybutanoyl)glycyl)pyrrolidine-2-carboxylate (7)

To a mixture of methyl (2S,4R)-4-isopropoxypyrrolidine-2-carboxylate hydrochloride (65 mg, 0.28 mmol) and (4-phenoxybutanamido)acetic acid (63 mg, 0.27 mmol) in DMF (3 mL) was added DIPEA (207 mg, 1.60 mmol) and T₃P (510 mg, 0.80 mmol, 50% wt. in ethyl acetate) at 0° C. under N₂ atmosphere. The reaction mixture was stirred at room temperature overnight before diluted with ethyl acetate and washed with saturated aq.NaHCO₃ solution. The aqueous layer was extracted with ethyl acetate twice and the combined organic layers were washed with brine, dried over anhydrous Na₂SO₄, filtered and concentrated to dryness under reduced pressure. The residue was purified by flash chromatography on silica gel (petroleum ether:ethyl acetate=5:1) to afford methyl (2S,4R)-4-isopropoxy-1-[2-(4-phenoxybutanamido)acetyl]pyrrolidine-2-carboxylate (90 mg, yield 82.9%) as white solid. LC/MS (ESI) m/z: 407 (M+H)⁺.

### Step 7: (2S,4R)-4-isopropoxy-1-((4-phenoxybutanoyl)glycyl)pyrrolidine-2-carboxylic acid (8)

To a solution of methyl (2S,4R)-4-isopropoxy-1-[2-(4-phenoxybutanamido)acetyl]pyrrolidine-2-carboxylate (90 mg, 0.22 mmol) in THF/MeOH/H₂O (3 mL, v/v=4/1/1) was added LiOH (11 mg, 0.44 mmol) and the reaction mixture was stirred at 0° C. for 2 hours. The mixture was diluted with water (5 mL) and washed with EtOAc twice. The aqueous layer was acidified with 1N aq.HCl to pH-4 and extracted with ethyl acetate twice. The combined organic layers were washed with brine, dried over anhydrous Na₂SO₄, filtered and concentrated to dryness under reduced pressure to afford (2S,4R)-4-isopropoxy-1-[2-(4-phenoxybutanamido)acetyl]pyrrolidine-2-carboxylic acid (80 mg, yield 92.1%) as white solid. LC/MS (ESI) m/z: 393 (M+H)⁺.

### Step 8: (2S,4R)-N-((R)-1-(4-carbamimidoylthiophen-2-yl)ethyl)-4-isopropoxy-1-((4-phenoxybutanoyl)glycyl)pyrrolidine-2-carboxamide (Compound 166)

To a mixture of (2S,4R)-4-isopropoxy-1-[2-(4-phenoxybutanamido)acetyl]pyrrolidine-2-carboxylic acid (40 mg, 0.10 mmol) and 5-[(1R)-1-aminoethyl]thiophene-3-carboximidamide (17 mg, 0.10 mmol) in DMF (3 mL) was added DIPEA (66 mg, 0.51 mmol) and PyBop (80 mg, 0.15 mmol) at 0° C. under N₂ atmosphere and the reaction mixture was stirred at room temperature for 2 hours. The mixture was diluted with ethyl acetate and washed with saturated aq.NH₄Cl solution. The aqueous layer was extracted with ethyl acetate twice and the combined organic layers were washed with brine, dried over anhydrous Na₂SO₄, filtered and concentrated to dryness under reduced pressure. The residue was purified by prep-HPLC to give Compound 166 (6 mg, yield 10.9%) as white solid. ¹HNMR (400 MHz, CD₃OD) δ 8.54 (s, 1H), 8.22 (dd, J=4.3, 1.5 Hz, 1H), 7.53 (d, J=11.6 Hz, 1H), 7.26-7.21 (m, 2H), 6.89 (dd, J=7.5, 5.5 Hz, 3H), 5.27 (dq, J=13.9, 6.7 Hz, 1H), 4.55-4.46 (m, 1H), 4.31 (s, 1H), 4.09-3.96 (m, 4H), 3.74 (dd, J=12.2, 6.2 Hz, 2H), 3.59 (d, J=11.3 Hz, 1H), 2.47 (t, J=7.4 Hz, 2H), 2.32-2.23 (m, 1H), 2.11-2.02 (m, 3H), 1.60 (dd, J=23.4, 7.0 Hz, 3H), 1.16 (dd, J=6.1, 3.1 Hz, 6H). LC/MS (ESI) m/z: 544 (M+H)⁺.

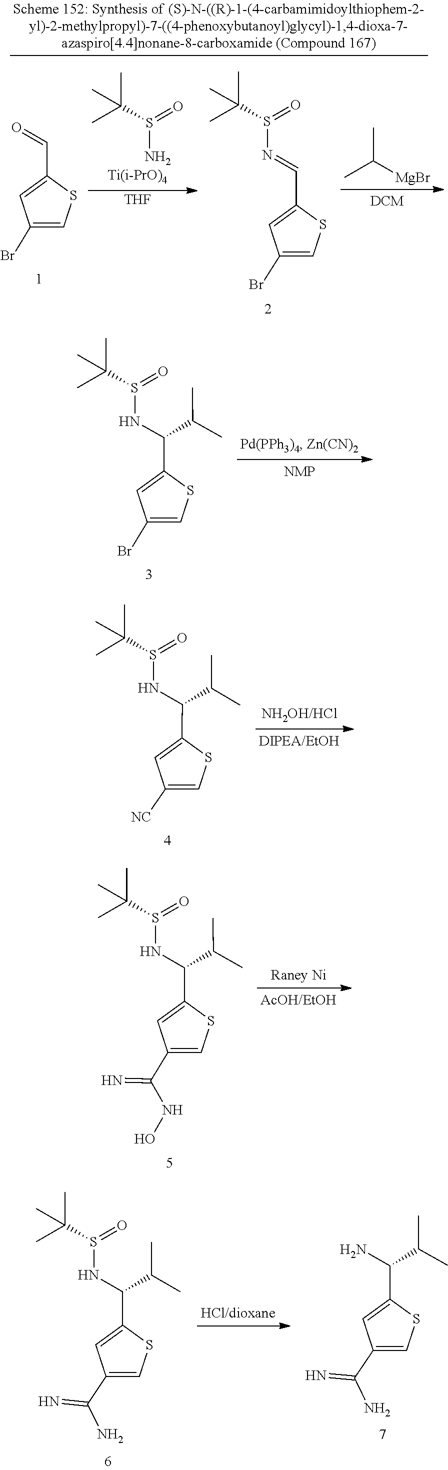

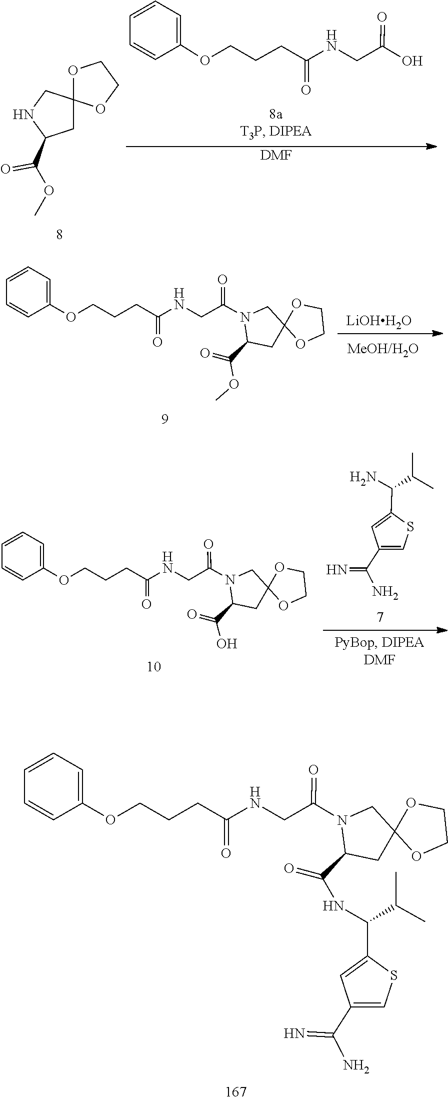

### Step 1: (S,E)-N-((4-bromothiophen-2-yl)methylene)-2-methylpropane-2-sulfinamide (2)

To a solution of 4-bromothiophene-2-carbaldehyde (5 g, 26.17 mmol) and (S)-2-methylpropane-2-sulfinamide (3.5 g, 28.79 mmol) in THE (20 mL) was added Ti(OiPr)₄ (11.2 g, 39.26 mmol). The reaction mixture was heated to 75° C. under N₂ atmosphere and stirred at 75° C. overnight. The mixture was diluted with ethyl acetate and washed with water. The aqueous layer was extracted with ethyl acetate twice and the combined organic layers were washed with brine, dried over anhydrous Na₂SO₄, filtered and concentrated to dryness under reduced pressure. The residue was purified by flash chromatography on silica gel (petroleum ether:ethyl acetate=1:1) to afford (S,E)-N-((4-bromothiophen-2-yl)methylene)-2-methylpropane-2-sulfinamide (6.2 g, Yield 80.0%) as white solid. LC/MS (ESI) m/z:294 (M+H)⁺.

### Step 2: (S)-N-((R)-1-(4-bromothiophen-2-yl)-2-methylpropyl)-2-methylpropane-2-sulfinamide (3)

To a solution of N-[(1E)-(4-bromothiophen-2-yl)methylidene]-2-methylpropane-2-sulfinamide (1.2 g, 4.08 mmol) in DCM (15 mL) was added bromo(isopropyl)magnesium (901 mg, 6.12 mmol) dropwise under N₂ atmosphere at −78° C., and the reaction mixture was stirred at room temperature overnight. The mixture was diluted with ethyl acetate and washed with saturated aq.NH₄Cl solution. The aqueous layer was extracted with ethyl acetate and the combined organic layers were washed with brine, dried over anhydrous Na₂SO₄, filtered and concentrated to dryness under reduced pressure. The residue was purified by flash column chromatography on silica gel (petroleum ether:ethyl acetate=5:1) to afford (S)-N-((R)-1-(4-bromothiophen-2-yl)-2-methylpropyl)-2-methylpropane-2-sulfinamide (650 mg, Yield 47.1%) as white solid. LC/MS (ESI) m/z:338 (M+H)⁺.

### Step 3: (S)-N-((R)-1-(4-cyanothiophen-2-yl)-2-methylpropyl)-2-methylpropane-2-sulfinamide (4)

To a solution of (S)-N-((R)-1-(4-bromothiophen-2-yl)-2-methylpropyl)-2-methylpropane-2-sulfinamide (650 mg, 1.92 mmol) in NMP (10 mL) was added Zn(CN)₂ (451 mg, 3.84 mmol) and Pd(PPh₃)₄ (222 mg, 0.19 mmol). The reaction mixture was heated to 160° C. under N₂ atmosphere and stirred at 160° C. overnight. The mixture was diluted with ethyl acetate and washed with water. The aqueous layer was extracted with ethyl acetate twice and the combined organic layers were washed with brine, dried over anhydrous Na₂SO₄, filtered and concentrated to dryness under reduced pressure. The residue was purified by flash column chromatography on silica gel (petroleum ether:ethyl acetate=5:1) to afford (S)-N-((R)-1-(4-cyanothiophen-2-yl)-2-methylpropyl)-2-methylpropane-2-sulfinamide (240 mg, Yield 44.0%) as white solid. LC/MS (ESI) m/z: 285 (M+H)⁺.

### Step 4: 5-((R)-1-(((S)-tert-butylsulfinyl)amino)-2-methylpropyl)-N-hydroxythiophene-3-carboximidamide (5)

To a solution of (S)-N-((R)-1-(4-cyanothiophen-2-yl)-2-methylpropyl)-2-methylpropane-2-sulfinamide (240 mg, 0.84 mmol) in EtOH (3 mL) was added NH₂OH hydrochloride (147 mg, 2.11 mmol) and DIPEA (327 mg, 2.53 mmol) under N₂ atmosphere and the reaction mixture was stirred at room temperature for 3 hours. The mixture was diluted with ethyl acetate and washed with water. The aqueous layer was extracted with ethyl acetate twice and the combined organic layers were washed with brine, dried over anhydrous Na₂SO₄, filtered and concentrated to dryness under reduced pressure. The residue was purified by flash column chromatography on silica gel (dichloromethane:methanol=10:1) to afford 5-((R)-1-(((S)-tert-butylsulfinyl)amino)-2-methylpropyl)-N-hydroxythiophene-3-carboximidamide (70 mg, Yield 26.1%) as white solid. LC/MS (ESI) m/z:318 (M+H)⁺.

### Step 5: 5-((R)-1-(((S)-tert-butylsulfinyl)amino)-2-methylpropyl)thiophene-3-carboximidamide (6)

To a solution of 5-((R)-1-(((S)-tert-butylsulfinyl)amino)-2-methylpropyl)-N-hydroxythiophene-3-carboximidamide (70 mg, 0.22 mmol) in methanol (2 mL) was added Raney-Ni (1 mL) and AcOH (0.1 mL) under N₂ atmosphere and the reaction mixture was stirred at room temperature for 3 hours. The mixture was concentrated to dryness under reduced pressure to afford 5-((R)-1-(((S)-tert-butylsulfinyl)amino)-2-methylpropyl)thiophene-3-carboximidamide (60 mg, Yield 90.3%) as white solid. LC/MS (ESI) m/z:302 (M+H)⁺.

### Step 6: (R)-5-(1-amino-2-methylpropyl)thiophene-3-carboximidamide (7)

To a solution of 5-((R)-1-(((S)-tert-butylsulfinyl)amino)-2-methylpropyl)thiophene-3-carboximidamide (60 mg, 0.20 mmol) in 1,4-dioxane (3 mL) was added HCl/1,4-dioxane (1 mL) under N₂ atmosphere and the reaction mixture was stirred at room temperature for 2 hours. The mixture was concentrated to dryness under reduced pressure to afford (R)-5-(1-amino-2-methylpropyl)thiophene-3-carboximidamide hydrochloride (36 mg, Yield 91.7%) as the white solid. LC/MS (ESI) m/z:198 (M+H)⁺.

### Step 7: methyl (S)-7-((4-phenoxybutanoyl)glycyl)-1,4-dioxa-7-azaspiro[4.4]nonane-8-carboxylate (9)

To a solution of methyl (S)-1,4-dioxa-7-azaspiro[4.4]nonane-8-carboxylate (280 mg, 1.50 mmol) and (4-phenoxybutanoyl)glycine (355 mg, 1.50 mmol) in DMF (10 mL) was added DIPEA (1.2 g, 8.98 mmol) and T₃P (2.9 g, 4.49 mmol, 50% wt in ethyl acetate) at 0° C. under N₂ atmosphere and the mixture was stirred at room temperature overnight. The mixture was diluted with ethyl acetate and washed with saturated aq.NaHCO₃ solution. The aqueous layer was extracted with ethyl acetate twice and the combined organic layers were washed with brine, dried over anhydrous Na₂SO₄, filtered and concentrated to dryness under reduced pressure. The residue was purified by flash column chromatography on silica gel (petroleum ether:ethyl acetate=3:1) to afford methyl (S)-7-((4-phenoxybutanoyl)glycyl)-1,4-dioxa-7-azaspiro[4.4]nonane-8-carboxylate (300 mg, Yield 49.3%) as white solid. LC/MS (ESI) m/z:407 (M+H)⁺.

### Step 8: (S)-7-((4-phenoxybutanoyl)glycyl)-1,4-dioxa-7-azaspiro[4.4]nonane-8-carboxylic acid (10)

To a solution of methyl (S)-7-((4-phenoxybutanoyl)glycyl)-1,4-dioxa-7-azaspiro[4.4]nonane-8-carboxylate (50 mg, 0.12 mmol) in THF:MeOH:H₂O=4:1:1 (3 mL) was added LiOH (6 mg, 0.25 mmol) under N₂ atmosphere and the reaction mixture was stirred at room temperature for 2 hours. The mixture was diluted with ethyl acetate and washed with water. The aqueous layer was extracted with ethyl acetate twice and the combined organic layers were washed with brine, dried over anhydrous Na₂SO₄, filtered and concentrated to dryness under reduced pressure. The residue was purified by flash column chromatography on silica gel (dichloromethane:methanol=10:1) to afford (S)-7-((4-phenoxybutanoyl)glycyl)-1,4-dioxa-7-azaspiro[4.4]nonane-8-carboxylic acid (40 mg, Yield 82.9%) as white solid. LC/MS (ESI) m/z:393 (M+H)⁺.

### Step 9: (S)-N-((R)-1-(4-carbamimidoylthiophen-2-yl)-2-methylpropyl)-7-((4-phenoxybutanoyl)glycyl)-1,4-dioxa-7-azaspiro[4.4]nonane-8-carboxamide (Compound167)

To a solution of (S)-7-((4-phenoxybutanoyl)glycyl)-1,4-dioxa-7-azaspiro[4.4]nonane-8-carboxylic acid (36 mg, 0.092 mmol) and (R)-5-(1-amino-2-methylpropyl) thiophene-3-carboximidamide (18 mg, 0.092 mmol) in DMF (3 mL) at 0° C. was added DIPEA (59 mg, 0.46 mmol) and PyBop (72 mg, 0.14 mmol) under N₂ atmosphere and the reaction mixture was stirred at room temperature for 2 hours. The mixture was diluted with ethyl acetate and washed with saturated aq.NaHCO₃ solution. The aqueous layer was extracted with ethyl acetate twice and the combined organic layers were washed with brine, dried over anhydrous Na₂SO₄, filtered and concentrated to dryness under reduced pressure. The residue was purified by prep-HPLC to give Compound 167 (0.6 mg, Yield 1.1%) as a white solid. ¹H-NMR (400 MHz, CD₃OD) δ 8.56 (s, 1H), 8.25 (d, J=1.3 Hz, 1H), 7.47 (s, 1H), 7.24 (t, J=7.9 Hz, 2H), 6.90 (dd, J=7.6, 5.6 Hz, 3H), 4.93 (s, 1H), 4.58 (t, J=7.4 Hz, 2H), 4.01-3.96 (m, 4H), 3.90 (dd, J=11.1, 6.0 Hz, 2H), 3.68 (t, J=6.4 Hz, 2H), 2.46 (t, J=7.4 Hz, 2H), 2.39 (dd, J=13.4, 9.2 Hz, 1H), 2.21-2.13 (m, 2H), 2.05 (dt, J=12.2, 6.5 Hz, 3H), 0.98-0.90 (m, 6H). LC/MS (ESI) m/z:572 (M+H)⁺.

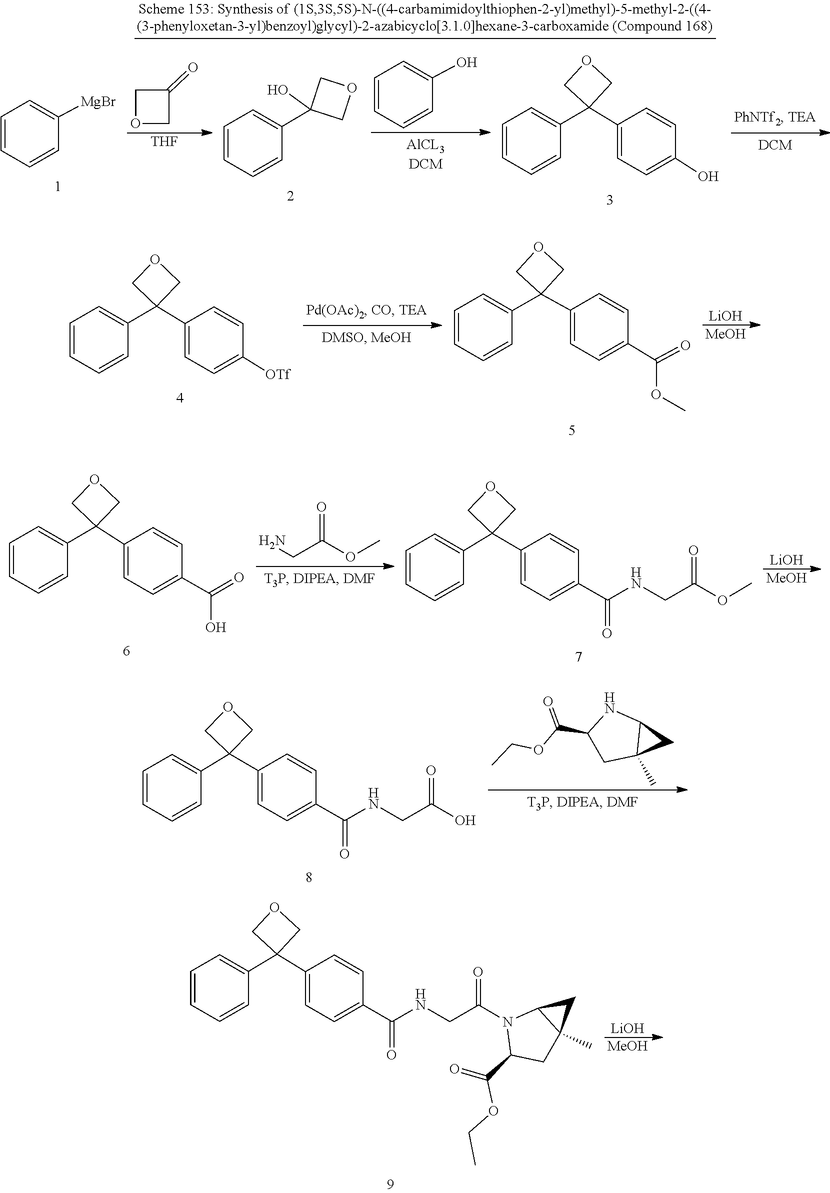

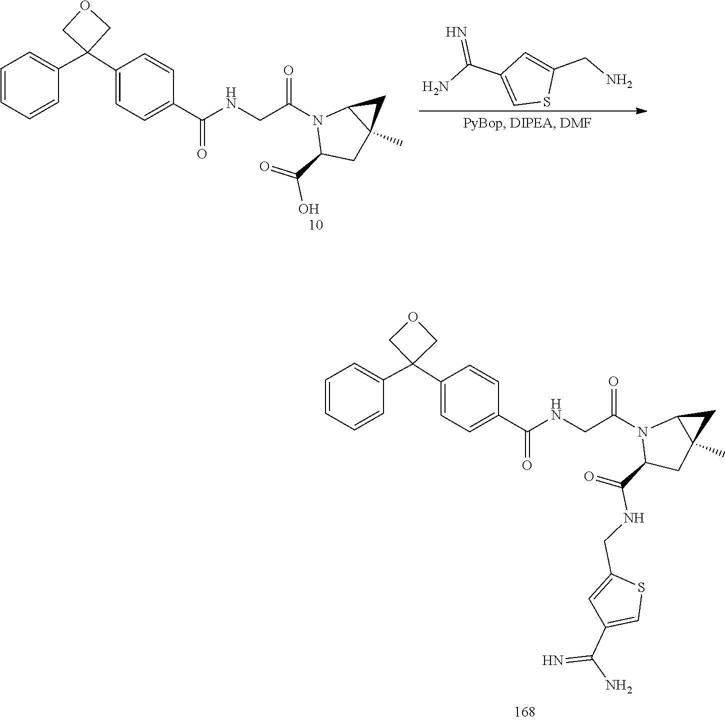 [SMILES: C[C@@]12C[C@@H]1N(C(=O)CNC(=O)c1ccc(C3(c4ccccc4)COC3)cc1)[C@H](C(=O)O)C2 ; N=C(N)c1csc(CN)c1 ; C[C@@]12C[C@@H]1N(C(=O)CNC(=O)c1ccc(C3(c4ccccc4)COC3)cc1)[C@H](C(=O)NCc1cc(C(=N)N)cs1)C2]

### Step 1:3-phenyloxetan-3-ol (2)

To a solution of oxetan-3-one (5 g, 69.4 mmol) in THE (25 mL) was added phenyl magnesium bromide (83 mL, 1 M in THF) at −60° C. and the mixture was stirred at room temperature for 2 hours. The mixture was quenched with sat.NH₄Cl solution and extracted with ethyl acetate twice. The combined organic layers were dried over anhydrous Na₂SO₄, filtered and concentrated to dryness under reduced pressure. The residue was purified by flash column chromatography on silica gel (PE:ethyl acetate=1:1) to give 3-phenyloxetan-3-ol (8 g, yield 76.8%) as colorless oil. LC/MS (ESI) m/z: 151 (M+H)⁺.

### Step 2: 4-(3-phenyloxetan-3-yl)phenol (3)

To a mixture of 3-phenyloxetan-3-ol (8 g, 53.3 mmol) and phenol (6 g, 63.8 mmol) in DCM (160 mL) was added AlCl₃ (7 g, 52.6 mmol) at 0° C. and the mixture was stirred at room temperature for 2 hours. The mixture was diluted with water and extracted with ethyl acetate twice. The combined organic layers were dried over anhydrous Na₂SO₄, filtered and concentrated to dryness under reduced pressure. The residue was purified by flash column chromatography on silica gel (PE:ethyl acetate=3:1) to give 4-(3-phenyloxetan-3-yl)phenol (5 g, yield 41.6%) as light yellow oil. LC/MS (ESI) m/z: 227 (M+H)⁺.

### Step 3: 4-(3-phenyloxetan-3-yl)phenyl trifluoromethanesulfonate (4)

To a solution of 4-(3-phenyloxetan-3-yl)phenol (3 g, 13.2 mmol) in DCM (100 mL) was added TEA (4 g, 39.6 mmol) and PhNTf₂ (7 g, 19.6 mmol) at 0° C. and the mixture was stirred at room temperature for 12 hours. The mixture was diluted with water and extracted with DCM twice. The combined organic layers were dried over anhydrous Na₂SO₄, filtered and concentrated to dryness under reduced pressure. The residue was purified by flash column chromatography on silica gel (PE:ethyl acetate=5:1) to give 4-(3-phenyloxetan-3-yl)phenyl trifluoromethanesulfonate (5 g, yield 100%) as colorless oil. LC/MS (ESI) m/z: 359 (M+H)⁺.

### Step 4: methyl 4-(3-phenyloxetan-3-yl)benzoate (5)

To a solution of 4-(3-phenyloxetan-3-yl)phenyl trifluoromethanesulfonate (1 g, 2.79 mmol) in DMSO (10 mL) and MeOH (10 mL) was added TEA (2 g, 19.8 mmol) and Pd(OAc)₂ (160 mg, 0.71 mmol) and the mixture was stirred under CO atmosphere at 70° C. for 4 hours. The mixture was diluted with water and extracted with DCM twice. The combined organic layers were dried over anhydrous Na₂SO₄, filtered and concentrated to dryness under reduced pressure. The residue was purified by flash column chromatography on silica gel (PE:ethyl acetate=4:1) to give methyl 4-(3-phenyloxetan-3-yl)benzoate (510 mg, yield 68.1%) as white solid. LC/MS (ESI) m/z: 269 (M+H)⁺.

### Step 5: methyl 4-(3-phenyloxetan-3-yl)benzoate (6)

To a solution of methyl 4-(3-phenyloxetan-3-yl)benzoate (150 mg, 1.86 mmol) in MeOH (5 mL) and water (1 mL) was added lithium hydroxide (60 mg, 2.50 mmol) at 0° C. and the mixture was stirred at room temperature for 2 hours. The mixture was acidified with 1N aq.HCl solution to pH-3 and extracted with DCM two times. The combined organic layers were washed with brine, dried over anhydrous Na₂SO₄, filtered and concentrated to dryness under reduced pressure to give 4-(3-phenyloxetan-3-yl)benzoic acid (400 mg, yield 84.5%) as white solid which was used directly in the next step. LC/MS (ESI) (m/z): 255 (M+H)⁺.

### Step 6: methyl (4-(3-phenyloxetan-3-yl)benzoyl)glycinate (7)

To a mixture of 4-(3-phenyloxetan-3-yl)benzoic acid (400 mg, 1.57 mmol) and methyl glycinate (180 mg, 2.02 mmol) in DMF (3.0 mL) was added DIPEA (1.2 g, 9.30 mmol) and T₃P (3 g, 9.43 mmol, 50% wt in ethyl acetate) at 0° C. under N₂ atmosphere and the mixture was stirred at 25° C. for 16 hours. The mixture was diluted with water and extracted with ethyl acetate twice. The combined organic layers were dried over anhydrous Na₂SO₄, filtered and concentrated to dryness under reduced pressure. The residue was purified by flash column chromatography on silica gel (petroleum ether:ethyl acetate=1:1) to give methyl (4-(3-phenyloxetan-3-yl) benzoyl)glycinate (400 mg, yield 78.2%) as white solid. LC/MS (ESI) m/z: 326 (M+H)⁺.

### Step 7: (4-(3-phenyloxetan-3-yl)benzoyl)glycine (8)

To a solution of methyl (4-(3-phenyloxetan-3-yl)benzoyl)glycinate (400 mg, 1.23 mmol) in MeOH (3 mL) and water (0.6 mL) was added lithium hydroxide (50 mg, 2.08 mmol) at 0° C. and the mixture was stirred at room temperature for 2 hours. The mixture was acidified with 1N aq.HCl to pH-3 and extracted with DCM twice. The combined organic layers were washed with brine, dried over anhydrous Na₂SO₄, filtered and concentrated to dryness under reduced pressure to give (4-(3-phenyloxetan-3-yl)benzoyl)glycine (300 mg, yield 78.5%) as white solid which was used directly in the next step. LC/MS (ESI) (m/z): 312 (M+H)⁺.

### Step 8: ethyl (1S,3S,5S)-5-methyl-2-((4-(3-phenyloxetan-3-yl)benzoyl)glycyl)-2-azabicyclo [3.1.0]hexane-3-carboxylate (9)

To a mixture of (4-(3-phenyloxetan-3-yl)benzoyl)glycine (120 mg, 0.38 mmol) and ethyl (1S,3S,5S)-5-methyl-2-azabicyclo[3.1.0]hexane-3-carboxylate (65 mg, 0.38 mmol) in DMF (3 mL) was added DIPEA (300 mg, 2.32 mmol) followed by T₃P (360 mg, 1.13 mmol, 50% in ethyl acetate) at 0° C., and the mixture was stirred at 25° C. for 1 hour. The resulting mixture was diluted with water (10 mL) and extracted with ethyl acetate (3×10 mL). The combined organic layers were washed with brine (2×10 mL), dried over anhydrous Na₂SO₄, filtered and concentrated to dryness under reduced pressure to give ethyl (1 S,3S,5S)-5-methyl-2-((4-(3-phenyloxetan-3-yl)benzoyl)glycyl)-2-azabicyclo[3.1.0]hexane-3-carboxylate (100 mg, Yield 56.1%) as yellow oil. LC/MS (ESI) m/z: 463 (M+H)⁺.

### Step 9: (1S,3S,5S)-5-methyl-2-((4-(3-phenyloxetan-3-yl)benzoyl)glycyl)-2-azabicyclo[3.1.0]hexane-3-carboxylic acid (10)

To a mixture of ethyl (1 S,3S,5S)-5-methyl-2-((4-(3-phenyloxetan-3-yl)benzoyl)glycyl)-2-azabicyclo[3.1.0]hexane-3-carboxylate(100 mg, 0.21 mmol) and LiOH·H₂O (34 mg, 0.81 mmol) in MeOH (2 mL) and water (1 mL) was stirred at 25° C. for 1 hour. The mixture was acidified with 0.5N HCl solution. The mixture was concentrated to dryness under reduced pressure to give (1 S,3S,5S)-5-methyl-2-((4-(3-phenyloxetan-3-yl)benzoyl)glycyl)-2-azabicyclo[3.1.0]hexane-3-carboxylic acid (50 mg, yield 52.8%) as white solid which was used directly in the next step. LC/MS (ESI) (m/z): 435 (M+H)⁺.

### Step 10: (1S,3S,5S)-N-((4-carbamimidoylthiophen-2-yl)methyl)-5-methyl-2-((4-(3-phenyloxetan-3-yl)benzoyl)glycyl)-2-azabicyclo[3.1.0]hexane-3-carboxamide (Compound 168)

To a mixture of (1 S,3S,5S)-5-methyl-2-((4-(3-phenyloxetan-3-yl)benzoyl)glycyl)-2-azabicyclo [3.1.0]hexane-3-carboxylic acid (50 mg, 0.11 mmol) and (R)-5-(aminomethyl)thiophene-3-carboximidamide (45 mg, 0.29 mmol) in DMF (2 mL) was added DIPEA (113 mg, 0.87 mmol) followed by PyBOP (243 mg, 0.46 mmol) at 0° C. and the mixture was stirred at 25° C. for 1 hour. The resulting mixture was diluted with water (10 mL) and extracted with ethyl acetate (3×10 mL). The combined organic layers were washed with brine (2×10 mL), dried over anhydrous Na₂SO₄, filtered and concentrated to dryness under reduced pressure to give the crude product which was further purified by prep-HPLC to afford Compound 168 (0.5 mg, Yield 1%) as white solid. ¹H-NMR (400 MHz, CD₃OD) δ 8.54 (s, 1H), 8.19 (d, J=1.6 Hz, 1H), 7.83 (d, J=8.5 Hz, 2H), 7.43-7.34 (m, 5H), 7.27 (d, J=7.6 Hz, 3H), 5.28 (dd, J=20.9, 6.0 Hz, 4H), 4.82 (s, 1H), 4.59-4.51 (m, 2H), 4.34 (d, J=5.4 Hz, 2H), 3.41 (dd, J=6.0, 2.5 Hz, 1H), 2.41 (t, J=12.4 Hz, 1H), 2.17 (dd, J=13.3, 3.2 Hz, 1H), 1.29 (s, 3H), 1.13 (dd, J=5.7, 2.5 Hz, 1H), 0.79 (t, J=6.5 Hz, 1H). LC/MS (ESI) m/z: 572 (M+H)⁺.

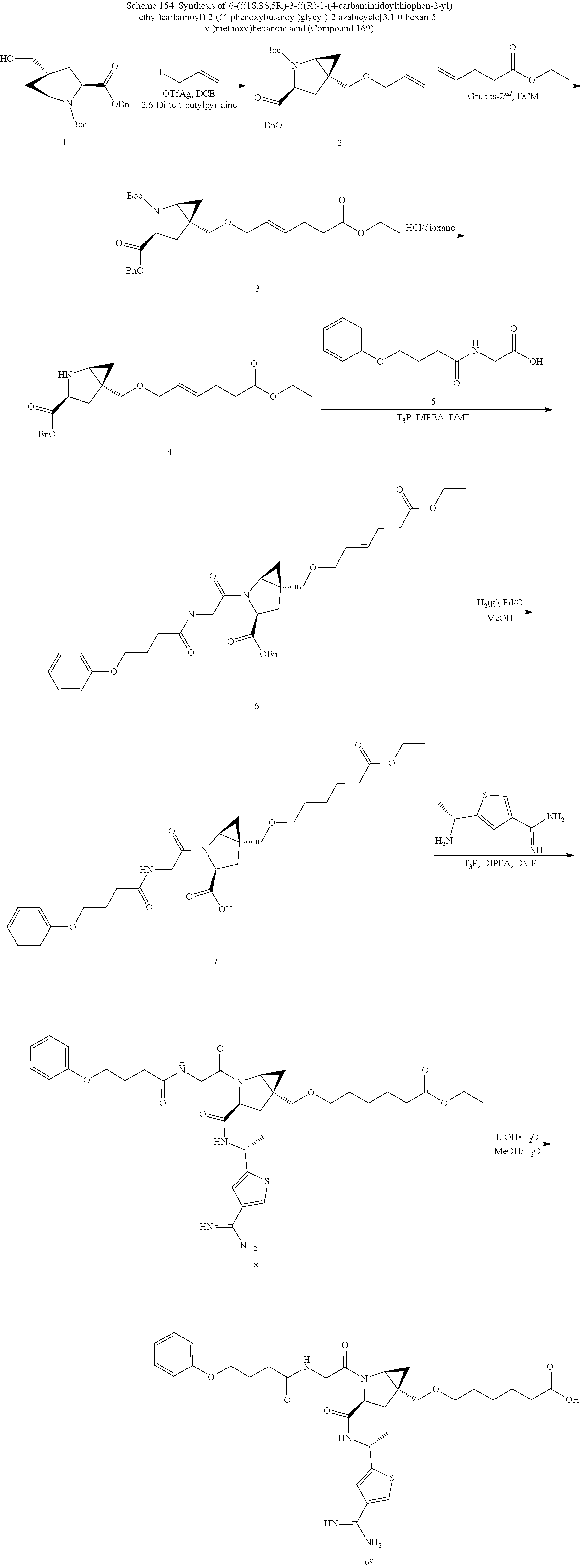

### Step 1: 3-benzyl 2-(tert-butyl) (1S,3S,5R)-5-((allyloxy)methyl)-2-azabicyclo[3.1.0]hexane-2,3-dicarboxylate (2)

To a solution of 3-benzyl 2-(tert-butyl) (1 S,3S,5R)-5-(hydroxymethyl)-2-azabicyclo[3.1.0]hexane-2,3-dicarboxylate (300 mg, 0.86 mmol) in DCE (5 mL) was added AgOTf (333 mg, 1.29 mmol) at 0° C., followed by 3-iodoprop-1-ene (0.24 mL, 2.58 mmol) and 2,6-di-tert-butyl pyridine (0.58 mL, 2.58 mmol) and the mixture was stirred under N₂ atmosphere at 30° C. for 16 hours. The mixture was filtered and concentrated to give the crude product which was purified by flash chromatography on silica gel (petroleum ether:ethyl acetate=5:1) to give 3-benzyl 2-(tert-butyl) (1S,3S,5R)-5-((allyloxy)methyl)-2-azabicyclo[3.1.0]hexane-2,3-dicarboxylate (200 mg, Yield 59.9%) as yellow oil.

LC/MS (ESI) (m/z): 388 (M+H)⁺.

### Step 2: methyl 3-benzyl 2-(tert-butyl)(1S,3S,5R)-5-((((E)-6-ethoxy-6-oxohex-2-en-1-yl)oxy) methyl)-2-azabicyclo[3.1.0]hexane-2,3-dicarboxylate (3)

To a solution of 3-benzyl 2-(tert-butyl) (1 S,3S,5R)-5-((allyloxy)methyl)-2-azabicyclo[3.1.0]hexane-2,3-dicarboxylate (200 mg, 0.52 mmol) in DCM (8.0 mL) was added Grubbs 2^{nd} (108 mg, 0.13 mmol) at 0° C. under N₂ atmosphere and the mixture was stirred at 25° C. for 16 hours. The mixture was concentrated to dryness under reduced pressure. The residue was purified by flash column chromatography on silica gel (petroleum ether:ethyl acetate=4:1) to give 3-benzyl 2-(tert-butyl) (1 S,3S,5R)-5-((((E)-6-ethoxy-6-oxohex-2-en-1-yl)oxy)methyl)-2-azabicyclo[3.1.0]hexane-2,3-dicarboxylate (100 mg, yield 39.7%) as brown oil. LC/MS (ESI) m/z: 488 (M+H)⁺.

### Step 3: benzyl (1S,3S,5R)-5-((((E)-6-ethoxy-6-oxohex-2-en-1-yl)oxy)methyl)-2-azabicyclo [3.1.0]hexane-3-carboxylate (4)

A solution of 3-benzyl 2-(tert-butyl) (1 S,3S,5R)-5-((((E)-6-ethoxy-6-oxohex-2-en-1-yl)oxy) methyl)-2-azabicyclo[3.1.0]hexane-2,3-dicarboxylate (100 mg, 0.21 mmol) in HCl/1,4-dioxane (3 mL, 4M) was stirred at room temperature for 1 hour. The reaction mixture was concentrated to dryness under reduced pressure, co-evaporated with DCM and dried under vacuum to give benzyl (1 S,3S,5R)-5-((((E)-6-ethoxy-6-oxohex-2-en-1-yl)oxy)methyl)-2-azabicyclo[3.1.0]hexane-3-carboxylate (75 mg, yield 93.8%) as brown oil, which was used directly in the next step without further purification. LC/MS (ESI) m/z: 388 (M+H)⁺.

### Step 4: benzyl (1S,3S,5R)-5-((((E)-6-ethoxy-6-oxohex-2-en-1-yl)oxy)methyl)-2-((4-phenoxybutanoyl)glycyl)-2-azabicyclo[3.1.0]hexane-3-carboxylate (6)

To a mixture of benzyl (1 S,3S,5R)-5-((((E)-6-ethoxy-6-oxohex-2-en-1-yl)oxy)methyl)-2-azabicyclo[3.1.0]hexane-3-carboxylate (75 mg, 0.19 mmol) and (4-phenoxybutanoyl)glycine (50 mg, 0.21 mmol) in DMF (3.0 mL) was added DIPEA (0.19 mL, 1.14 mmol) and T₃P (370 mg, 0.57 mmol, 50% wt in ethyl acetate) at 0° C. under N₂ atmosphere and the mixture was stirred at 25° C. for 16 hours. The mixture was diluted with water and extracted with ethyl acetate twice. The combined organic layers were dried over anhydrous Na₂SO₄, filtered and concentrated to dryness under reduced pressure. The residue was purified by flash column chromatography on silica gel (dichloromethane:methanol=96:4) to give methyl (2S,4R)-4-(difluoromethoxy)-1-((4-(4-fluorophenoxy)benzoyl)glycyl)pyrrolidine-2-carboxylate (60 mg, yield 51.2%) as yellow oil. LC/MS (ESI) m/z: 607 (M+H)⁺.

### Step 5: (1S,3S,5R)-5-(((6-ethoxy-6-oxohexyl)oxy)methyl)-2-((4-phenoxybutanoyl)glycyl)-2-azabicyclo[3.1.0]hexane-3-carboxylic acid (7)

To a solution of benzyl (1 S,3S,5R)-5-((((E)-6-ethoxy-6-oxohex-2-en-1-yl)oxy)methyl)-2-((4-phenoxybutanoyl)glycyl)-2-azabicyclo[3.1.0]hexane-3-carboxylate (60 mg, 0.10 mmol) in methanol (5.0 mL) was added Pd/C (10 mg, 10% wt) and the mixture was degassed under N₂ atmosphere for three times and stirred under a H₂ balloon at room temperature for 1 hour. The mixture was filtered and filtrate was concentrated to dryness under reduced pressure to give (1 S,3S,5R)-5-(((6-ethoxy-6-oxohexyl)oxy)methyl)-2-((4-phenoxybutanoyl)glycyl)-2-azabicyclo[3.1.0]hexane-3-carboxylic acid (50.0 mg, yield 98.0%) as yellow oil, which was used directly in next step. LC/MS (ESI) (m/z): 519 (M+H)⁺.

### Step 6: ethyl 6-(((1S,3S,5R)-3-(((R)-1-(4-carbamimidoylthiophen-2-yl)ethyl)carbamoyl)-2-((4-phenoxybutanoyl)glycyl)-2-azabicyclo[3.1.0]hexan-5-yl)methoxy)hexanoate (8)

To a mixture of (1S,3S,5R)-5-(((6-ethoxy-6-oxohexyl)oxy)methyl)-2-((4-phenoxybutanoyl) glycyl)-2-azabicyclo[3.1.0]hexane-3-carboxylic acid (50 mg, 0.10 mmol) and (R)-5-(1-aminoethyl)thiophene-3-carboximidamide (24 mg, 0.15 mmol) in DMF (3.0 mL) was added DIPEA (0.1 mL, 0.60 mmol) and T₃P (184 mg, 0.30 mmol, 50% wt in ethyl acetate) at 0° C. under N₂ atmosphere and the mixture was stirred at 25° C. for 16 hours. The mixture was concentrated to dryness under reduced pressure. The residue was purified by prep-TLC (dichloromethane:methanol=7:1) to give ethyl 6-(((1S,3S,5R)-3-(((R)-1-(4-carbamimidoylthiophen-2-yl)ethyl)carbamoyl)-2-((4-phenoxybutanoyl)glycyl)-2-azabicyclo[3.1.0]hexan-5-yl)methoxy)hexanoate (50 mg, yield 76.9%) as colorless oil. LC/MS (ESI) m/z: 670 (M+H)⁺.

### Step 7: 6-(((1S,3S,5R)-3-(((R)-1-(4-carbamimidoylthiophen-2-yl)ethyl)carbamoyl)-2-((4-phenoxybutanoyl)glycyl)-2-azabicyclo[3.1.0]hexan-5-yl)methoxy)hexanoic acid (Compound 169)

To a solution of ethyl 6-(((1 S,3S,5R)-3-(((R)-1-(4-carbamimidoylthiophen-2-yl)ethyl) carbamoyl)-2-((4-phenoxybutanoyl)glycyl)-2-azabicyclo[3.1.0]hexan-5-yl)methoxy)hexanoate (50 mg, 0.07 mmol) in methanol (3 mL) and water (0.6 mL) was added lithium hydroxide (4.7 mg, 0.11 mmol) at 0° C. and the mixture was stirred at room temperature for 2 hours. The mixture was acidified with 1N aq.HCl to pH-3. The residue was purified by prep-HPLC to give Compound 169 (5 mg, yield 10.6%) as a white solid. ¹H NMR (400 MHz, CD₃OD) δ 8.52 (s, 1H), 8.21 (d, J=1.6 Hz, 1H), 7.50 (s, 1H), 7.25-7.20 (m, 2H), 6.88 (dd, J=7.5, 5.7 Hz, 3H), 5.19 (q, J=7.4 Hz, 1H), 4.57 (s, 1H), 4.21 (d, J=16.6 Hz, 1H), 4.11 (d, J=16.6 Hz, 1H), 3.99 (t, J=6.3 Hz, 2H), 3.51 (d, J=10.4 Hz, 1H), 3.47 (dt, J=6.6, 3.9 Hz, 3H), 3.40 (d, J=10.3 Hz, 1H), 2.65 (t, J=12.9 Hz, 1H), 2.46 (t, J=7.4 Hz, 2H), 2.21 (t, J=7.4 Hz, 2H), 2.11-2.04 (m, 3H), 1.58 (dd, J=20.4, 7.1 Hz, 7H), 1.45-1.38 (m, 2H), 1.22 (dd, J=5.7, 2.7 Hz, 1H), 0.97 (t, J=6.3 Hz, 1H). LC/MS (ESI) m/z: 642 (M+H)⁺.

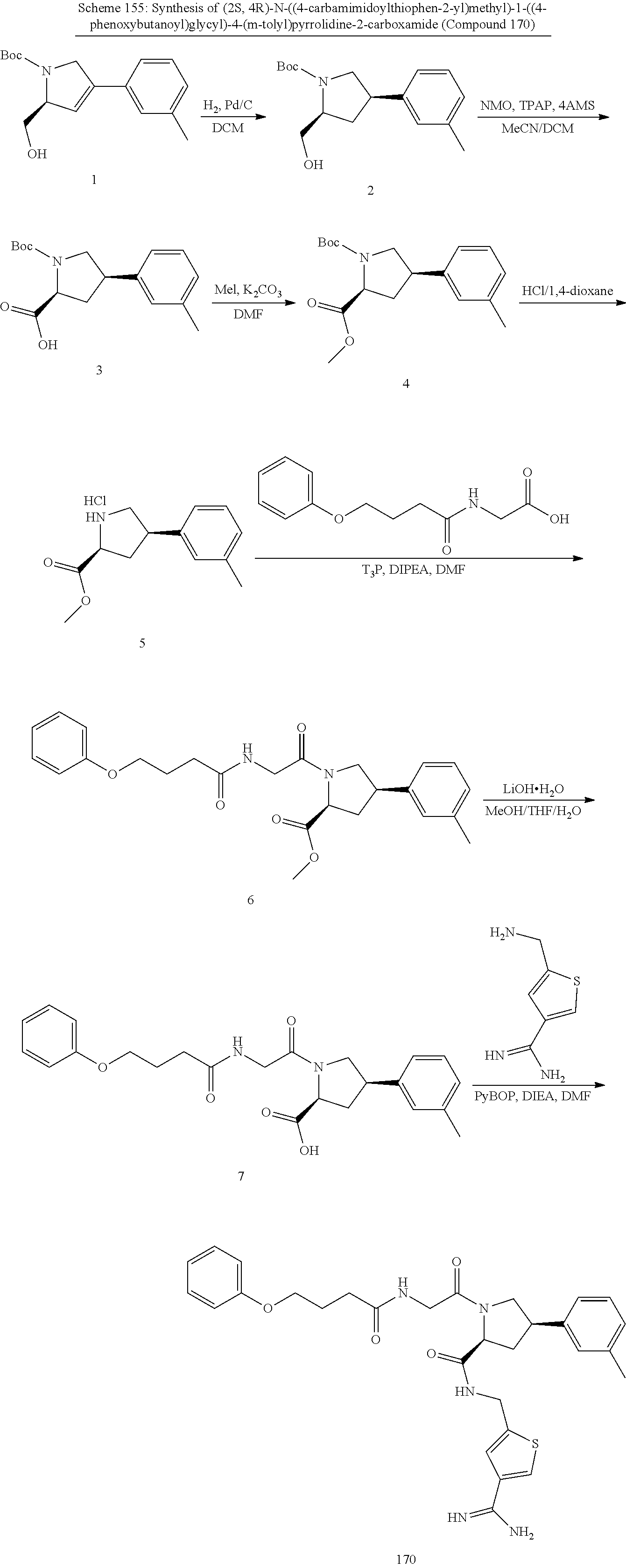

### Step 1: tert-butyl (2S,4R)-2-(hydroxymethyl)-4-(m-tolyl)pyrrolidine-1-carboxylate (2)

To a solution of (S)-tert-butyl 2-(hydroxymethyl)-4-(m-tolyl)-2,5-dihydro-1H-pyrrole-1-carboxylate (309 mg, 1.07 mmol) in methanol 4 mL) was added Pd/C (30 mg, 10% wt) at 0° C., and the reaction mixture was stirred at 25° C. under H₂ atmosphere for 4 hours. The mixture was filtered and concentrated to dryness under reduced pressure to give tert-butyl (2S,4R)-2-(hydroxymethyl)-4-(m-tolyl)pyrrolidine-1-carboxylate (278 mg, yield 89.4%) as light oil. LC/MS (ESI) m/z: 192 (M−100+H)⁺.

### Step 2: (2S,4R)-1-(tert-butoxycarbonyl)-4-(m-tolyl)pyrrolidine-2-carboxylic acid (3)

To a solution of tert-butyl(2S,4R)-2-(hydroxymethyl)-4-(m-tolyl)pyrrolidine-1-carboxylate (278 mg, 0.96 mmol) in dichloromethane (3 mL) and MeCN (0.3 mL) was added NMO (224 mg, 1.91 mmol) and 4 Å molecular sieves (224 mg) at 0° C. The mixture was stirred for 10 minutes before TPAP (34 mg, 0.10 mmol) was added and the reaction was stirred at 25° C. for 1 hour. The mixture was diluted with DCM, acidified with 1N HCl solution, and extracted with ethyl acetate twice. The combined organic layers were washed with brine, dried over anhydrous Na₂SO₄, filtered and concentrated to dryness under reduced pressure to give (2S,4R)-1-(tert-butoxycarbonyl)-4-(m-tolyl)pyrrolidine-2-carboxylic acid (100 mg, yield 34.2%) as light oil which was used directly in the next step. LC/MS (ESI) m/z: 304 (M−H)⁺

### Step 3: 1-(tert-butyl) 2-methyl (2S,4R)-4-(m-tolyl)pyrrolidine-1,2-dicarboxylate (4)

To a solution of (2S,4R)-1-(tert-butoxycarbonyl)-4-(m-tolyl)pyrrolidine-2-carboxylic acid (100 mg, 0.33 mmol) in DMF (2 mL) was added Cs₂CO₃ (118 mg, 0.36 mmol) followed by Mel (0.02 mL, 0.40 mmol) at 0° C., and the mixture was stirred at 25° C. for 5 hours. The mixture was extracted with ethyl acetate twice, and the combined organic layers were washed with brine, dried over anhydrous Na₂SO₄, filtered and concentrated to dryness under reduced pressure. The residue was purified by flash chromatography on silica gel (petroleum ether:ethyl acetate=5:1) to give 1-(tert-butyl) 2-methyl (2S,4R)-4-(m-tolyl)pyrrolidine-1,2-dicarboxylate (61 mg, yield 58.1%) as light oil. LC/MS (ESI) m/z: 220 (M−100+H)⁺

### Step 4: methyl (2S,4R)-4-(m-tolyl)pyrrolidine-2-carboxylate (5)

A solution of 1-(tert-butyl) 2-methyl (2S,4R)-4-(m-tolyl)pyrrolidine-1,2-dicarboxylate (61 mg, 0.19 mmol) in HCl/1,4-dioxane (2 mL) at 0° C. was stirred at room temperature for 2 hours. The reaction mixture was concentrated to dryness under reduced pressure, washed with DCM and dried under vacuum to give methyl (2S,4R)-4-(m-tolyl)pyrrolidine-2-carboxylate (41 mg, yield 99.1%) as yellow oil which was used directly in the next step. LC/MS (ESI) m/z: 220 (M+H)⁺

### Step 5: methyl (2S,4R)-1-((4-phenoxybutanoyl)glycyl)-4-(m-tolyl)pyrrolidine-2-carboxylate (6)

To a mixture of methyl (2S,4R)-4-(m-tolyl)pyrrolidine-2-carboxylate (41 mg, 0.19 mmol) and (4-phenoxybutanoyl)glycine (53 mg, 0.22 mmol) in DMF (2 mL) was added DIPEA (0.2 mL, 1.14 mmol) followed by T₃P (363 mg, 0.57 mmol, 50% wt in ethyl acetate) at 0° C., and the mixture was stirred at 30° C. for 16 hours. The mixture was washed with saturated aq.NaHCO₃, extracted with ethyl acetate twice and washed with brine, dried over anhydrous Na₂SO₄, filtered and concentrated to dryness under reduced pressure. The residue was purified by flash chromatography on silica gel (dichloromethane:methanol=20:1) to give methyl (2S,4R)-1-((4-phenoxybutanoyl) glycyl)-4-(m-tolyl)pyrrolidine-2-carboxylate (80 mg, yield 96.4%) as yellow oil. LC/MS (ESI) m/z: 439 (M+H)⁺.

### Step 6: (2S,4R)-1-((4-phenoxybutanoyl)glycyl)-4-(m-tolyl)pyrrolidine-2-carboxylic acid (7)

To a solution of methyl (2S,4R)-1-((4-phenoxybutanoyl) glycyl)-4-(m-tolyl)pyrrolidine-2-carboxylate (80 mg, 0.18 mmol) in methanol (1 mL) and THE (0.5 mL) was added a solution of LiOH·H₂O (8 mg, 0.18 mmol) in water (0.5 mL) at 0° C., and the mixture was stirred at 25° C. for 4 hours. The mixture was diluted with water and extracted with ethyl acetate twice. The aqueous layer was acidified with 1N HCl solution, extracted with ethyl acetate twice and the combined organic layers were washed with brine, dried over anhydrous Na₂SO₄, filtered and concentrated to dryness under reduced pressure to give (2S,4R)-1-((4-phenoxybutanoyl)glycyl)-4-(m-tolyl) pyrrolidine-2-carboxylic acid (66 mg, yield 86.8%) as light oil, which was used directly in the next step. LC/MS (ESI) m/z: 425 (M+H)⁺

### Step 7: (2S,4R)-N-((4-carbamimidoylthiophen-2-yl)methyl)-1-((4-phenoxybutanoyl)glycyl)-4-(m-tolyl)pyrrolidine-2-carboxamide (Compound 170)

To a mixture of (2S,4R)-1-((4-phenoxybutanoyl)glycyl)-4-(m-tolyl)pyrrolidine-2-carboxylic acid (66 mg, 0.16 mmol) and 5-(aminomethyl)thiophene-3-carboximidamide (30 mg, 0.19 mmol) in DMF (2 mL) was added DIPEA (0.1 mL, 0.80 mmol), followed by PyBOP (125 mg, 0.24 mmol) at 0° C., and the mixture was stirred at 25° C. for 30 minutes. The mixture was extracted with ethyl acetate twice, washed with brine, dried over anhydrous Na₂SO₄, filtered and concentrated to dryness under reduced pressure. The residue was purified by prep-HPLC to give Compound 170 (7 mg, yield 7.8%) as a white solid. ¹H NMR (400 MHz, CD₃OD) δ 8.22 (dd, J=3.2, 2.8 Hz, 1H), 7.49-7.41 (m, 1H), 7.26-7.19 (m, 3H), 7.15-7.06 (m, 3H), 6.91-6.87 (m, 3H), 4.60-4.56 (m, 2H), 4.55-4.44 (m, 1H), 4.18-4.08 (m, 2H), 3.99 (dd, J=12.4, 10.4 Hz, 3H), 3.60 (t, J=10.4 Hz, 1H), 3.52-3.42 (m, 1H), 2.88-2.52 (m, 1H), 2.47 (t, J=7.5 Hz, 2H), 2.32 (s, 3H), 2.11-1.98 (m, 3H). LC/MS (ESI) m/z: 562 (M+H)⁺.

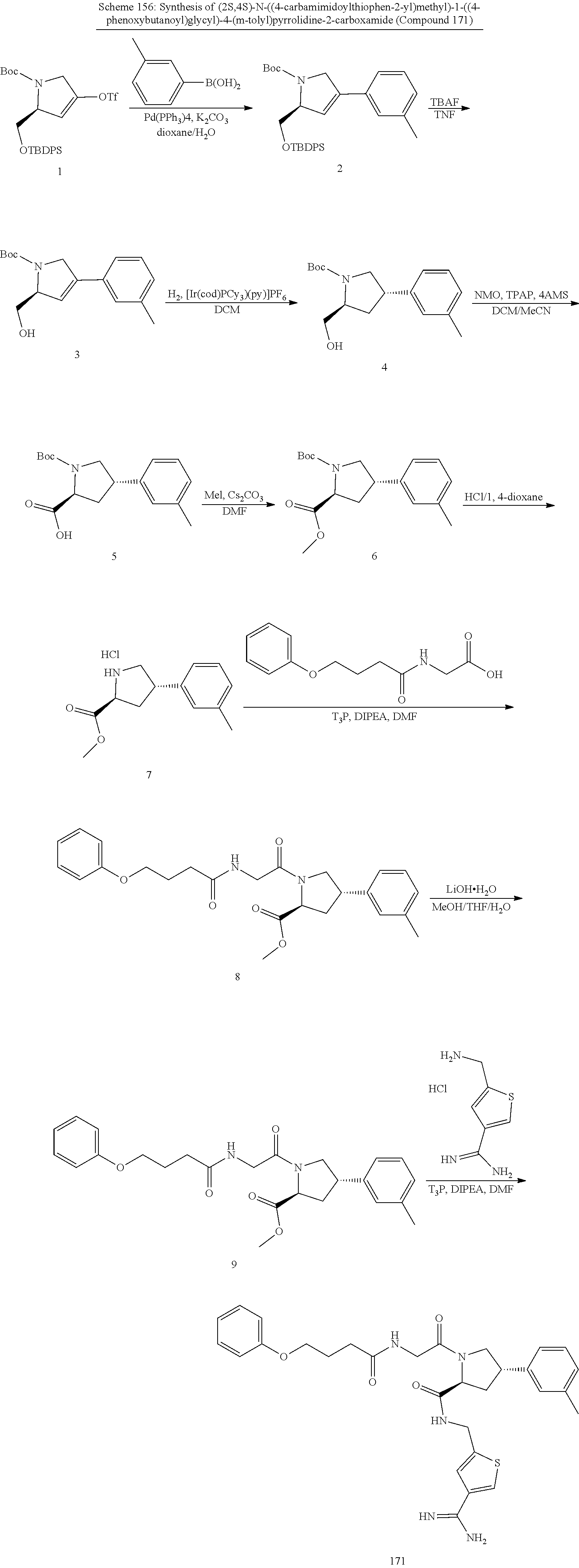

### Step 1: tert-butyl (S)-2-(((tert-butyldiphenylsilyl)oxy)methyl)-4-(m-tolyl)-2,5-dihydro-1H-pyrrole-1-carboxylate (2)

To a mixture of (S)-tert-butyl 2-(((tert-butyldiphenylsilyl)oxy)methyl)-4-(((trifluoromethyl) sulfonyl)oxy)-2,5-dihydro-1H-pyrrole-1-carboxylate (2 g, 3.42 mmol) and m-tolylboronic acid (465 mg, 3.42 mmol) in 1,4-dioxane (14 mL) and water (2 mL) was added K₂CO₃ (1.2 g, 8.55 mmol) followed by Pd(PPh₃)₄ (395 mg, 0.34 mmol) at 0° C., and the mixture was stirred at 90° C. for 4 hours. The mixture was diluted with ethyl acetate twice and washed with brine, dried over anhydrous Na₂SO₄, filtered and concentrated to dryness under reduced pressure. The residue was purified by flash chromatography on silica gel (petroleum ether:ethyl acetate=10:1) to give (S)-tert-butyl 2-(((tert-butyldiphenylsilyl)oxy)methyl)-4-(m-tolyl)-2,5-dihydro-1H-pyrrole-1-carboxylate (1.5 g, yield 83.3%) as light oil. LC/MS (ESI) m/z: 471 (M−56+H)⁺.

### Step 2: tert-butyl (S)-2-(hydroxymethyl)-4-(m-tolyl)-2,5-dihydro-1H-pyrrole-1-carboxylate (3)

To a solution of (S)-tert-butyl 2-(((tert-butyldiphenylsilyl)oxy)methyl)-4-(m-tolyl)-2,5-dihydro-1H-pyrrole-1-carboxylate (1.5 g, 2.80 mmol) in THE (15 mL) was added TBAF (5.7 mL, 1M in TBAF) at 0° C., and the mixture was stirred at room temperature for 3 hours. The mixture was diluted with ethyl acetate twice and washed with brine, dried over anhydrous Na₂SO₄, filtered and concentrated to dryness under reduced pressure. The residue was purified by flash chromatography on silica gel (petroleum ether:ethyl acetate=5:1) to give (S)-tert-butyl 2-(hydroxymethyl)-4-(m-tolyl)-2,5-dihydro-1H-pyrrole-1-carboxylate (738 mg, yield 91.2%) as yellow oil. LC/MS (ESI) m/z: 190 (M−100+H)⁺.

### Step 3: tert-butyl (2S,4S)-2-(hydroxymethyl)-4-(m-tolyl)pyrrolidine-1-carboxylate (4)

To a solution of (S)-tert-butyl 2-(hydroxymethyl)-4-(m-tolyl)-2,5-dihydro-1H-pyrrole-1-carboxylate (429 mg, 1.48 mmol) in DCM (5 mL) was added [Ir(cod)(PCy₃)(py)]PF₆ (24 mg, 0.03 mmol) at 0° C., and the reaction mixture was stirred at 25° C. under H₂ atmosphere for 4 days. The mixture was filtered and concentrated to dryness under reduced pressure. The crude residue was purified by flash chromatography on silica gel (petroleum ether:ethyl acetate=1:1) to give (2S,4S)-tert-butyl 2-(hydroxymethyl)-4-(m-tolyl) pyrrolidine-1-carboxylate (359 mg, yield 83.5%) as yellow oil. LC/MS (ESI) m/z: 192 (M−100+H)⁺.

### Step 4: (2S,4S)-1-(tert-butoxycarbonyl)-4-(m-tolyl)pyrrolidine-2-carboxylic acid (5)

To a solution of (2S,4S)-tert-butyl 2-(hydroxymethyl)-4-(m-tolyl)pyrrolidine-1-carboxylate (359 mg, 1.23 mmol) in DCM (3 mL) and MeCN (0.3 mL) was added NMO (288 mg, 2.46 mmol) and 4 Å molecular sieves (288 mg) at 0° C. The mixture was stirred for 10 minutes and TPAP (43 mg, 0.12 mmol) was added. The reaction was stirred at 25° C. for 1 hour before diluted with DCM, and the aqueous layer was acidified with 1N HCl solution, extracted with ethyl acetate twice. The combined organic layers were washed with brine, dried over anhydrous Na₂SO₄, filtered and concentrated to dryness under reduced pressure to give (2S,4S)-1-(tert-butoxycarbonyl)-4-(m-tolyl)pyrrolidine-2-carboxylic acid (128 mg, yield 34.1%) as yellow oil which was used directly in the next step LC/MS (ESI) m/z: 304 (M−H)⁺

### Step 5: 1-(tert-butyl) 2-methyl (2S,4S)-4-(m-tolyl)pyrrolidine-1,2-dicarboxylate (6)

To a solution of give (2S,4S)-1-(tert-butoxycarbonyl)-4-(m-tolyl)pyrrolidine-2-carboxylic acid (120 mg, 0.39 mmol) in DMF (2 mL) was added Cs₂CO₃ (141 mg, 0.43 mmol) followed by Mel (0.03 mL, 0.47 mmol) at 0° C., and the mixture was stirred at 25° C. for 4.5 hours. The mixture was extracted with ethyl acetate twice, and the combined organic layers were washed with brine, dried over anhydrous Na₂SO₄, filtered and concentrated to dryness under reduced pressure. The residue was purified by flash chromatography on silica gel (petroleum ether:ethyl acetate=5:1) to give 1-(tert-butyl) 2-methyl (2S,4S)-4-(m-tolyl)pyrrolidine-1,2-dicarboxylate (80 mg, yield 64.5%) as light oil. LC/MS (ESI) m/z: 220 (M−100+H)⁺

### Step 6: methyl (2S,4S)-4-(m-tolyl)pyrrolidine-2-carboxylate (7)

A solution of 1-(tert-butyl) 2-methyl (2S,4S)-4-(m-tolyl)pyrrolidine-1,2-dicarboxylate (80 mg, 0.25 mmol) in HCl/1,4-dioxane (2 mL) was stirred at room temperature for 4 hours. The reaction mixture was concentrated to dryness under reduced pressure, diluted with DCM and dried under vacuum to give methyl (2S,4S)-4-(m-tolyl)pyrrolidine-2-carboxylate (54 mg, yield 99.9%) as yellow solid which was used directly in the next step. LC/MS (ESI) m/z: 220 (M+H)⁺

### Step 7: methyl (2S,4S)-1-((4-phenoxybutanoyl)glycyl)-4-(m-tolyl)pyrrolidine-2-carboxylate (8)

To a mixture of methyl (2S,4S)-4-(m-tolyl)pyrrolidine-2-carboxylate (53 mg, 0.24 mmol) and (4-phenoxybutanoyl)glycine (69 mg, 0.29 mmol) in DMF (2 mL) was added DIPEA (0.2 mL, 1.44 mmol) followed by T₃P (458 mg, 0.72 mmol, 50% wt in ethyl acetate) at 0° C., and the mixture was stirred at 35° C. for 3 hours. The mixture was washed with saturated aq.NaHCO₃, extracted with ethyl acetate twice and washed with brine, dried over anhydrous Na₂SO₄, filtered and concentrated to dryness under reduced pressure. The residue was purified by flash chromatography on silica gel (dichloromethane:methanol=20:1) to give methyl (2S,4S)-1-((4-phenoxybutanoyl)glycyl)-4-(m-tolyl)pyrrolidine-2-carboxylate (99 mg, yield 94.3%) as yellow oil. LC/MS (ESI) m/z: 439 (M+H)⁺.

### Step 8: (2S,4S)-1-((4-phenoxybutanoyl)glycyl)-4-(m-tolyl)pyrrolidine-2-carboxylic acid (9)

To a solution of methyl (2S,4S)-1-((4-phenoxybutanoyl)glycyl)-4-(m-tolyl)pyrrolidine-2-carboxylate (99 mg, 0.23 mmol) in methanol (1 mL) and THE (0.5 mL) was added a solution of LiOH·H₂O (9 mg, 0.23 mmol) in water (0.5 mL) at 0° C., and the mixture was stirred at 25° C. for 4 hours. The mixture was diluted with water and extracted with ethyl acetate twice. The aqueous layer was acidified with 1N HCl solution, extracted with ethyl acetate twice. The combined organic layers were washed with brine, dried over anhydrous Na₂SO₄, filtered and concentrated to dryness under reduced pressure to give (2S,4S)-1-((4-phenoxybutanoyl)glycyl)-4-(m-tolyl)pyrrolidine-2-carboxylic acid (46 mg, yield 47.4%) as light oil, which was used directly in the next step. LC/MS (ESI) m/z: 425 (M+H)⁺

### Step 9: (2S,4S)-N-((4-carbamimidoylthiophen-2-yl)methyl)-1-((4-phenoxybutanoyl)glycyl)-4-(m-tolyl)pyrrolidine-2-carboxamide (Compound 171)

To a mixture of (2S,4S)-1-((4-phenoxybutanoyl)glycyl)-4-(m-tolyl) pyrrolidine-2-carboxylic acid (46 mg, 0.11 mmol) and 5-(aminomethyl)thiophene-3-carboximidamide (25 mg, 0.13 mmol) in DMF (2 mL) was added DIPEA (0.1 mL, 0.66 mmol) followed by T₃P (209 mg, 0.33 mmol, 50% wt in ethyl acetate) at 0° C., and the mixture was stirred at 35° C. for 4 hours. The mixture was washed with saturated aq.NaHCO₃, extracted with ethyl acetate twice and washed with brine, dried over anhydrous Na₂SO₄, filtered and concentrated to dryness under reduced pressure. The residue was purified by prep-HPLC to give Compound 171 (6 mg, yield 9.8%) as a white solid. ¹H NMR (400 MHz, CD₃OD) δ 8.55 (s, 1H), 8.21 (d, J=10.4 Hz, 1H), 7.46 (d, J=26.0 Hz, 1H), 7.27-7.18 (m, 4H), 7.08 (dd, J=20.8, 18.0 Hz, 2H), 6.92-6.86 (m, 3H), 4.65 (d, J=6.4 Hz, 1H), 4.58 (d, J=9.6 Hz, 2H), 4.14 (dd, J=23.6, 16.4 Hz, 2H), 4.00 (dd, J=10.0, 10.8 Hz, 3H), 3.59 (d, J=5.2 Hz, 1H), 3.48 (s, 1H), 2.50-2.45 (m, 2H), 2.33 (t, J=8.4 Hz, 4H), 2.16-1.98 (m, 3H).LC/MS (ESI) m/z: 562 (M+H)⁺.

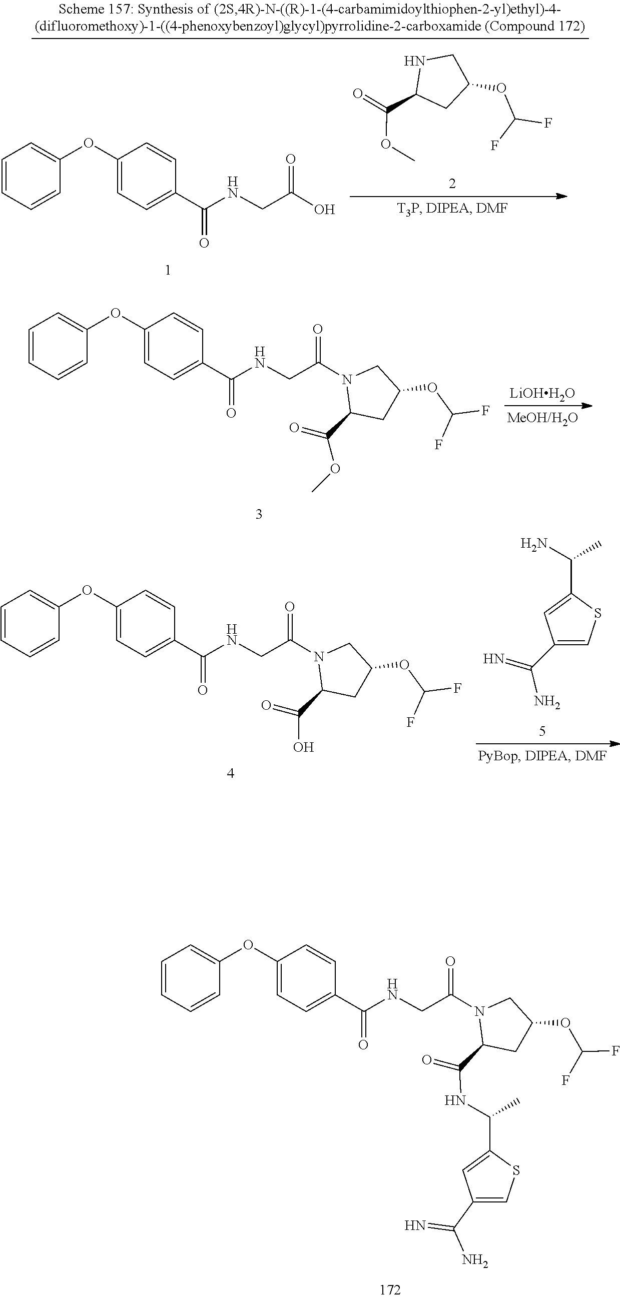

### Step 1: methyl (2S,4R)-4-(difluoromethoxy)-1-((4-phenoxybenzoyl)glycyl)pyrrolidine-2-carboxylate

To a mixture of (4-phenoxybenzoyl)glycine (110 mg, 0.42 mmol) and methyl (2S,4R)-4-(difluoromethoxy)pyrrolidine-2-carboxylate (90 mg, 0.46 mmol) in DMF (1.5 mL) was added DIPEA (325 mg, 2.52 mmol) and T₃P (400 mg, 1.26 mmol, 50% in ethyl acetate) at 0° C. under N₂ atmosphere and the reaction mixture was stirred at room temperature for 1 hour. The mixture was diluted with water and extracted with ethyl acetate twice. The combined organic layers were washed with brine, dried over anhydrous Na₂SO₄, filtered and concentrated to dryness under reduced pressure. The residue was purified by flash column chromatography on silica gel (petroleum ether:ethyl acetate=1:1) to afford methyl (2S,4R)-4-(difluoromethoxy)-1-((4-phenoxybenzoyl)glycyl)pyrrolidine-2-carboxylate (116 mg, yield 61.6%) as white solid. LC/MS (ESI) m/z: 449 (M+H)⁺.

### Step 2: (2S,4R)-4-(difluoromethoxy)-1-((4-phenoxybenzoyl)glycyl)pyrrolidine-2-carboxylic acid

To a mixture of methyl (2S,4R)-4-(difluoromethoxy)-1-((4-phenoxybenzoyl)glycyl) pyrrolidine-2-carboxylate (116 mg, 0.26 mmol) in CH₃₀H (1.5 mL), THE (0.5 ml) and water (0.5 mL) was added LiOH·H₂O (33 mg, 0.78 mmol) at 0° C. and the mixture was stirred under N₂ atmosphere at room temperature for 1 hour. The mixture was acidified with 1N aq.HCl solution to pH<3 and extracted with ethyl acetate twice. The combined organic layers were concentrated to dryness under reduced pressure to afford (2S,4R)-4-(difluoromethoxy)-1-((4-phenoxybenzoyl)glycyl)pyrrolidine-2-carboxylic acid (94 mg, 83.4% yield) as white solid, which was used directly in next step without further purification. LC/MS (ESI) (m/z): 435(M+H)⁺.

### Step 3: (2S,4R)-N-((R)-1-(4-carbamimidoylthiophen-2-yl)ethyl)-4-(difluoromethoxy)-1-((4-phenoxybenzoyl)glycyl)pyrrolidine-2-carboxamide (Compound 172)

To a mixture of (2S,4R)-4-(difluoromethoxy)-1-((4-(4-(trifluoromethyl)phenoxy) benzoyl)glycyl)pyrrolidine-2-carboxylic acid (80 mg, 0.18 mmol) and (R)-5-(1-aminoethyl) thiophene-3-carboximidamide (40 mg, 0.24 mmol) in DMF (1 mL) was added DIPEA (142 mg, 1.10 mmol) and PyBop (124 mg, 0.24 mmol) at 0° C. under N₂ atmosphere and the reaction mixture was stirred at room temperature for 1 hour. The mixture was diluted with water and extracted with ethyl acetate twice. The combined organic layers were washed with brine, dried over anhydrous Na₂SO₄, filtered and concentrated to dryness under reduced pressure. The residue was purified by flash column chromatography on silica gel (dichloromethane:methyl alcohol=10:1) to afford Compound 172 (2.8 mg, yield 2.6%) as a white solid. ¹H NMR (400 MHz, CD₃OD) δ 8.22 (d, J=1.6 Hz, 1H), 7.85 (d, J=8.9 Hz, 2H), 7.52 (s, 1H), 7.44-7.39 (m, 2H), 7.21 (d, J=7.4 Hz, 1H), 7.06 (d, J=7.6 Hz, 2H), 7.01 (d, J=8.9 Hz, 2H), 6.51 (s, 1H), 5.02 (s, 1H), 4.57 (s, 1H), 4.26 (d, J=16.7 Hz, 1H), 4.14 (d, J=16.6 Hz, 1H), 3.94 (s, 1H), 3.88 (s, 1H), 3.13 (s, 1H), 2.19 (s, 3H), 1.60 (s, 2H). LC/MS (ESI) (m/z): 586(M+H)⁺.

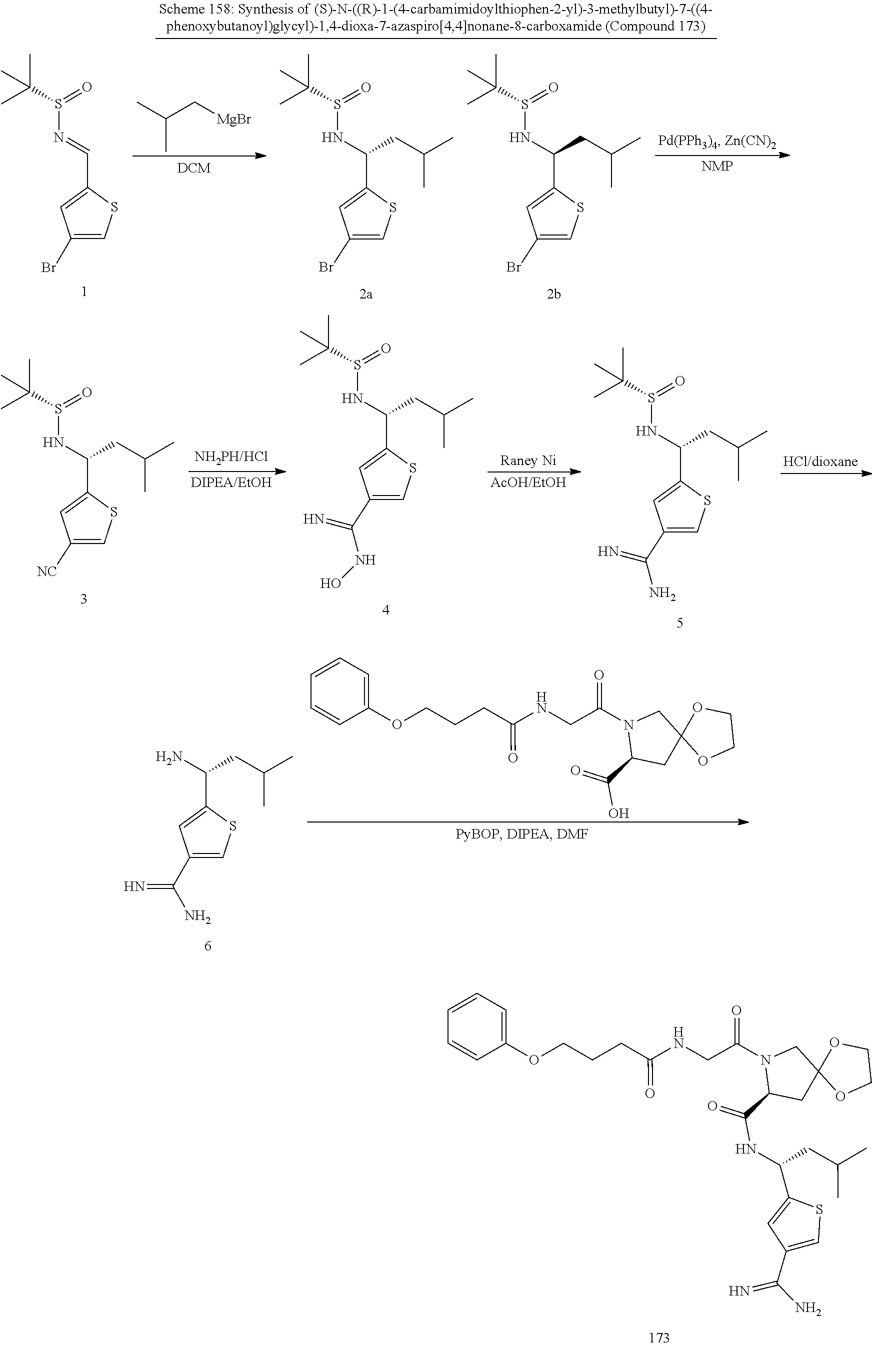

### Step 1: (S)-N-((R)-1-(4-bromothiophen-2-yl)-3-methylbutyl)-2-methylpropane-2-sulfinamide

To a solution of (S,E)-N-((4-bromothiophen-2-yl)methylene)-2-methylpropane-2-sulfinamide (1.0 g, 3.40 mmol) in THE (10 mL) was added cyclopentylmagnesium bromide (6.8 mL, 6.80 mmol) dropwise at −78° C. under N₂ atmosphere and the reaction mixture was stirred at −78° C. for 2 hours. The mixture was diluted with ethyl acetate and washed with saturated aq.NH₄Cl solution. The aqueous layer was extracted with ethyl acetate twice and the combined organic layers were washed with brine, dried over anhydrous Na₂SO₄, filtered and concentrated to dryness under reduced pressure. The residue was purified by flash column chromatography on silica gel (petroleum ether:ethyl acetate=80:1) to afford (S)-N-((R)-1-(4-bromothiophen-2-yl)-3-methylbutyl)-2-methylpropane-2-sulfinamide (280 mg, yield 23.5%) and (S)-N-((S)-1-(4-bromothiophen-2-yl)-3-methylbutyl)-2-methylpropane-2-sulfinamide (220 mg, yield 18.4%) as light oil. LC/MS (ESI) m/z: 352/354 (M+H)⁺.

### Step 2: (S)-N-((R)-1-(4-cyanothiophen-2-yl)-3-methylbutyl)-2-methylpropane-2-sulfinamide

To a solution of (S)-N-((R)-1-(4-bromothiophen-2-yl)-3-methylbutyl)-2-methylpropane-2-sulfinamide (280 mg, 0.80 mmol) in NMP (8 mL) was added Zn(CN)₂ (281 mg, 2.40 mmol) and Pd(PPh₃)₄ (185 mg, 0.16 mmol). The reaction mixture was heated to 160° C. under N₂ atmosphere and the reaction mixture was stirred at 160° C. for 2 hours. The mixture was diluted with ethyl acetate and washed with water. The aqueous layer was extracted with ethyl acetate twice and the combined organic layers were washed with brine, dried over anhydrous Na₂SO₄, filtered and concentrated to dryness under reduced pressure. The residue was purified by flash column chromatography on silica gel (petroleum ether:ethyl acetate=50:1) to afford (S)-N-((R)-1-(4-cyanothiophen-2-yl)-3-methylbutyl)-2-methylpropane-2-sulfinamide (88 mg, yield 37.0%) as light oil. LC/MS (ESI) m/z: 299 (M+H)⁺.

### Step 3: 5-((R)-1-(((S)-tert-butylsulfinyl)amino)-3-methylbutyl)-N-hydroxythiophene-3-carboximidamide

To a mixture of (S)-N-((R)-1-(4-cyanothiophen-2-yl)-3-methylbutyl)-2-methylpropane-2-sulfinamide (88 mg, 0.30 mmol) in EtOH (3 mL) was added DIPEA (0.15 mL, 0.89 mmol) and NH₂OH·HCl (48 mg, 0.69 mmol) and the mixture was stirred at 25° C. for 16 hours. The reaction mixture was quenched with ice water and extracted with DCM twice, the combined organic layers were washed with brine, dried over anhydrous Na₂SO₄ and concentrated to dryness under reduced pressure. The residue was purified by flash column chromatography on silica gel (petroleum ether:ethyl acetate=2:1) to give 5-((R)-1-(((S)-tert-butylsulfinyl)amino)-3-methylbutyl)-N-hydroxythiophene-3-carboximidamide (54 mg, yield 55.2%) as colorless oil. LC/MS (ESI) m/z: 332(M+H)⁺.

### Step 4: 5-((R)-1-(((S)-tert-butylsulfinyl)amino)-3-methylbutyl)thiophene-3-carboximidamide

To a solution of 5-((R)-1-(((S)-tert-butylsulfinyl)amino)-3-methylbutyl)-N-hydroxythiophene-3-carboximidamide (50 mg, 0.15 mmol) in MeOH (3 mL) and AcOH (0.02 ml) was added Raney Nickel (10 mg) at 0° C., and the mixture was degassed under N₂ atmosphere for three times and stirred under a H₂ balloon at room temperature overnight. The mixture was filtered and the filtrate was concentrated to dryness under reduced pressure to give 5-((R)-1-(((S)-tert-butylsulfinyl)amino)-3-methylbutyl)thiophene-3-carboximidamide (45 mg, yield 95.0%) as green oil, which was used directly in the next step without further purification. LC/MS (ESI) (m/z): 316 (M+H)⁺.

### Step 5: (R)-5-(1-amino-3-methylbutyl)thiophene-3-carboximidamide

A mixture of 5-((R)-1-(((S)-tert-butylsulfinyl)amino)-3-methylbutyl)thiophene-3-carboximidamide (45 mg, 0.14 mmol) in HCl/1,4-dioxane (2 mL, 4M) was stirred at room temperature for 1 hour. The reaction mixture was concentrated to dryness under reduced pressure, co-evaporated with DCM and dried under vacuum to give (R)-5-(1-amino-3-methylbutyl)thiophene-3-carboximidamide (30 mg, yield 96.9%) as white solid, which was used directly in the next step without further purification. LC/MS (ESI) m/z: 212 (M+H)⁺.

### Step 6: (S)-N-((R)-1-(4-carbamimidoylthiophen-2-yl)-3-methylbutyl)-7-((4-phenoxybutanoyl) glycyl)-1,4-dioxa-7-azaspiro[4.4]nonane-8-carboxamide (Compound 173)

To a mixture of (R)-5-(1-amino-3-methylbutyl)thiophene-3-carboximidamide (30 mg, 0.14 mmol) and (S)-7-((4-phenoxybutanoyl)glycyl)-1,4-dioxa-7-azaspiro[4.4]nonane-8-carboxylic acid (25 mg, 0.13 mmol) in DMF (3 mL) was added DIPEA (0.14 mL, 0.85 mmol) and T₃P (271 mg, 0.42 mmol, 50% wt in ethyl acetate) at 0° C. under N₂ atmosphere and the mixture was stirred at 25° C. for 3 hours. The mixture was washed with saturated aq. NaHCO₃ solution. The mixture was quenched with water and extracted with (CHCl₃:isopropyl alcohol=3:1) twice. The combined organic layers were washed with brine, dried over anhydrous Na₂SO₄, filtered and concentrated to dryness under reduced pressure. The residue was purified by prep-TLC (dichloromethane:methanol=10:1) and prep-HPLC to give Compound 173 (1.0 mg, yield 1.2%) as a white solid. ¹H NMR (400 MHz, CD₃OD) δ 8.22 (d, J=1.2 Hz, 1H), 7.50 (s, 1H), 7.23 (t, J=8.0 Hz, 2H), 6.89 (dd, J=3.2, 6.4 Hz, 3H), 5.29-5.19 (m, 1H), 4.61-4.50 (m, 2H), 4.02-3.89 (m, 8H), 3.73-3.65 (m, 2H), 2.50-2.36 (m, 3H), 2.10 (m, J=6.0, 16.8, 6.8 Hz, 3H), 1.73-1.63 (m, 2H), 1.00-0.92 (m, 6H). LC/MS (ESI) (m/z):586 (M+H)⁺.

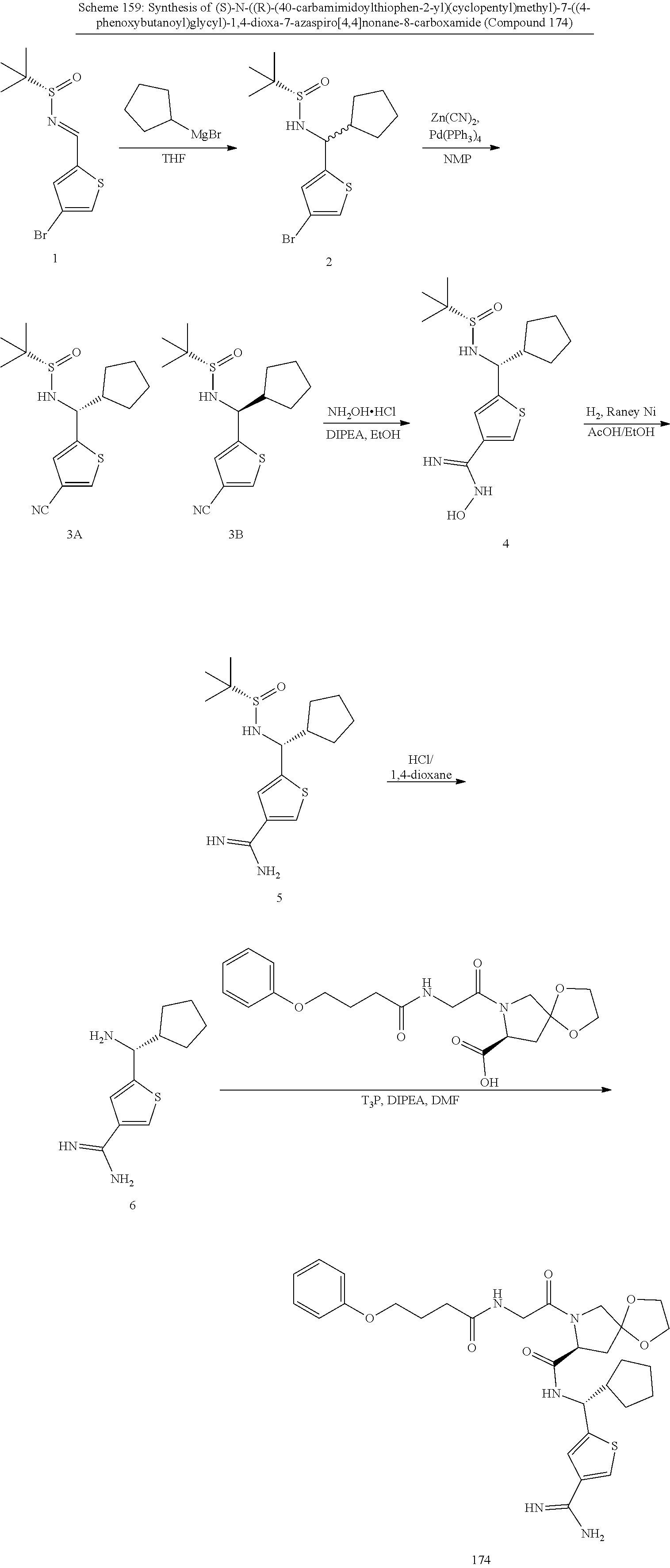

### Step 1: (S)-N-((4-bromothiophen-2-yl)(cyclopentyl)methyl)-2-methylpropane-2-sulfinamide

To a solution of (S,E)-N-((4-bromothiophen-2-yl)methylene)-2-methylpropane-2-sulfinamide (1.0 g, 3.40 mmol) in THE (10 mL) was added cyclopentylmagnesium bromide (6.8 mL, 6.80 mmol) dropwise at −78° C. under N₂ atmosphere and the reaction mixture was stirred at −78° C. for 2 hours. The mixture was diluted with ethyl acetate and washed with saturated aq.NH₄Cl solution. The aqueous layer was extracted with ethyl acetate twice and the combined organic layers were washed with brine, dried over anhydrous Na₂SO₄, filtered and concentrated to dryness under reduced pressure. The residue was purified by flash column chromatography on silica gel (petroleum ether:ethyl acetate=10:1) to give (S)-N-((4-bromothiophen-2-yl)(cyclopentyl)methyl)-2-methylpropane-2-sulfinamide (600 mg, yield 48.7%) as light oil. LC/MS (ESI) m/z: 364/366 (M+H)⁺.

### Step 2: (S)-N-((R)-(4-cyanothiophen-2-yl)(cyclopentyl)methyl)-2-methylpropane-2-sulfinamide

To a solution of (S)-N-((4-bromothiophen-2-yl)(cyclopentyl)methyl)-2-methylpropane-2-sulfinamide (600 mg, 1.65 mmol) in NMP (8 mL) was added Zn(CN)₂ (579 mg, 4.95 mmol) and Pd(PPh₃)₄ (381 mg, 0.33 mmol). The reaction mixture was heated to 160° C. under N₂ atmosphere and the reaction mixture was stirred at 160° C. for 2 hours. The mixture was diluted with ethyl acetate and washed with water. The aqueous layer was extracted with ethyl acetate twice and the combined organic layers were washed with brine, dried over anhydrous Na₂SO₄, filtered and concentrated to dryness under reduced pressure. The residue was purified by flash column chromatography on silica gel (dichloromethane:methanol=200:1) to give (S)-N-((R)-(4-cyanothiophen-2-yl)(cyclopentyl)methyl)-2-methylpropane-2-sulfinamide (150 mg, yield 29.4%) and (S)-N-((S)-(4-cyanothiophen-2-yl)(cyclopentyl)methyl)-2-methylpropane-2-sulfinamide(120 mg, yield 23.5%) as light oil. LC/MS (ESI) m/z: 311 (M+H)⁺.

### Step 3: 5-((R)-(((S)-tert-butylsulfinyl)amino)(cyclopentyl)methyl)-N-hydroxythiophene-3-carboximidamide

To a solution of (S)-N-((R)-(4-cyanothiophen-2-yl)(cyclopentyl)methyl)-2-methylpropane-2-sulfinamide (150 mg, 0.48 mmol) in EtOH (3 mL) was added NH₂OH hydrochloride (84 mg, 1.21 mmol) and DIPEA (186 mg, 1.44 mmol) under N₂ atmosphere and the reaction mixture was stirred at room temperature overnight. The mixture was diluted with dichloromethane and washed with water. The aqueous layer was extracted with dichloromethane twice and the combined organic layers were washed with brine, dried over anhydrous Na₂SO₄, filtered and concentrated to dryness under reduced pressure. The residue was purified by flash column chromatography on silica gel (dichloromethane:methanol=100:1) to give 5-((R)-(((S)-tert-butylsulfinyl)amino)(cyclopentyl)methyl)-N-hydroxythiophene-3-carboximidamide (70 mg, yield 42.5%) as white solid. LC/MS (ESI) m/z:344 (M+H)⁺.

### Step 4: 5-((R)-(((S)-tert-butylsulfinyl)amino)(cyclopentyl)methyl)thiophene-3-carboximidamide

To a solution of 5-((R)-(((S)-tert-butylsulfinyl)amino)(cyclopentyl)methyl)-N-hydroxythiophene-3-carboximidamide (70 mg, 0.20 mmol) in methanol (3 mL) was added Raney Nickel (20 mg) and AcOH (one drop) under H₂ atmosphere and the reaction mixture was stirred at room temperature overnight. The mixture was concentrated to dryness under reduced pressure to give 5-((R)-(((S)-tert-butylsulfinyl)amino)(cyclopentyl)methyl)thiophene-3-carboximidamide (65 mg, yield 99.4%) as white solid. LC/MS (ESI) m/z:328 (M+H)⁺.

### Step 5: (R)-5-(amino(cyclopentyl)methyl)thiophene-3-carboximidamide

A solution of 5-((R)-(((S)-tert-butylsulfinyl)amino)(cyclopentyl)methyl)thiophene-3-carboximidamide (65 mg, 0.20 mmol) in HCl/1,4-dioxane (2 mL, 4M) under N₂ atmosphere was stirred at room temperature for 1 hour. The mixture was concentrated to dryness under reduced pressure to give (R)-5-(amino(cyclopentyl) methyl)thiophene-3-carboximidamide (40 mg, yield 90.9%) as white solid.

LC/MS (ESI) m/z:224 (M+H)⁺.

### Step 6: (S)-N-((R)-(4-carbamimidoylthiophen-2-yl)(cyclopentyl)methyl)-7-((4-phenoxybutanoyl)glycyl)-1,4-dioxa-7-azaspiro[4.4]nonane-8-carboxamide (Compound 174)

To a solution of (R)-5-(amino(cyclopentyl)methyl)thiophene-3-carboximidamide (40 mg, 0.18 mmol) and (S)-7-((4-phenoxybutanoyl)glycyl)-1,4-dioxa-7-azaspiro[4.4]nonane-8-carboxylic acid (70 mg, 0.18 mmol) in DMF (2 mL) was added DIPEA (116 mg, 0.90 mmol) and T₃P (172 mg, 0.27 mmol, 50% wt in ethyl acetate) at 0° C. under N₂ atmosphere and the mixture was stirred at room temperature for 1 hour. The mixture was diluted with ethyl acetate and washed with saturated aq.NaHCO₃ solution. The aqueous layer was extracted with ethyl acetate twice and the combined organic layers were washed with brine, dried over anhydrous Na₂SO₄, filtered and concentrated to dryness under reduced pressure. The residue was purified by prep-HPLC to give Compound 174 (10 mg, yield 9.3%) as a white solid. ¹H NMR (400 MHz, CD₃OD) δ 8.25-8.20 (m, 1H), 7.51-7.46 (m, 1H), 7.26-7.20 (m, 2H), 6.90-6.86 (m, 3H), 4.99-4.90 (m, 1H), 4.63-4.50 (m, 1H), 4.01-3.86 (m, 8H), 3.71-3.64 (m, 2H), 2.48-2.40 (m, 3H), 2.40-2.32 (m, 1H), 2.15-2.03 (m, 3H), 1.91-1.77 (m, 1H), 1.69-1.54 (m, 5H), 1.36-1.22 (m, 2H). LC/MS (ESI) m/z:598 (M+H)⁺.

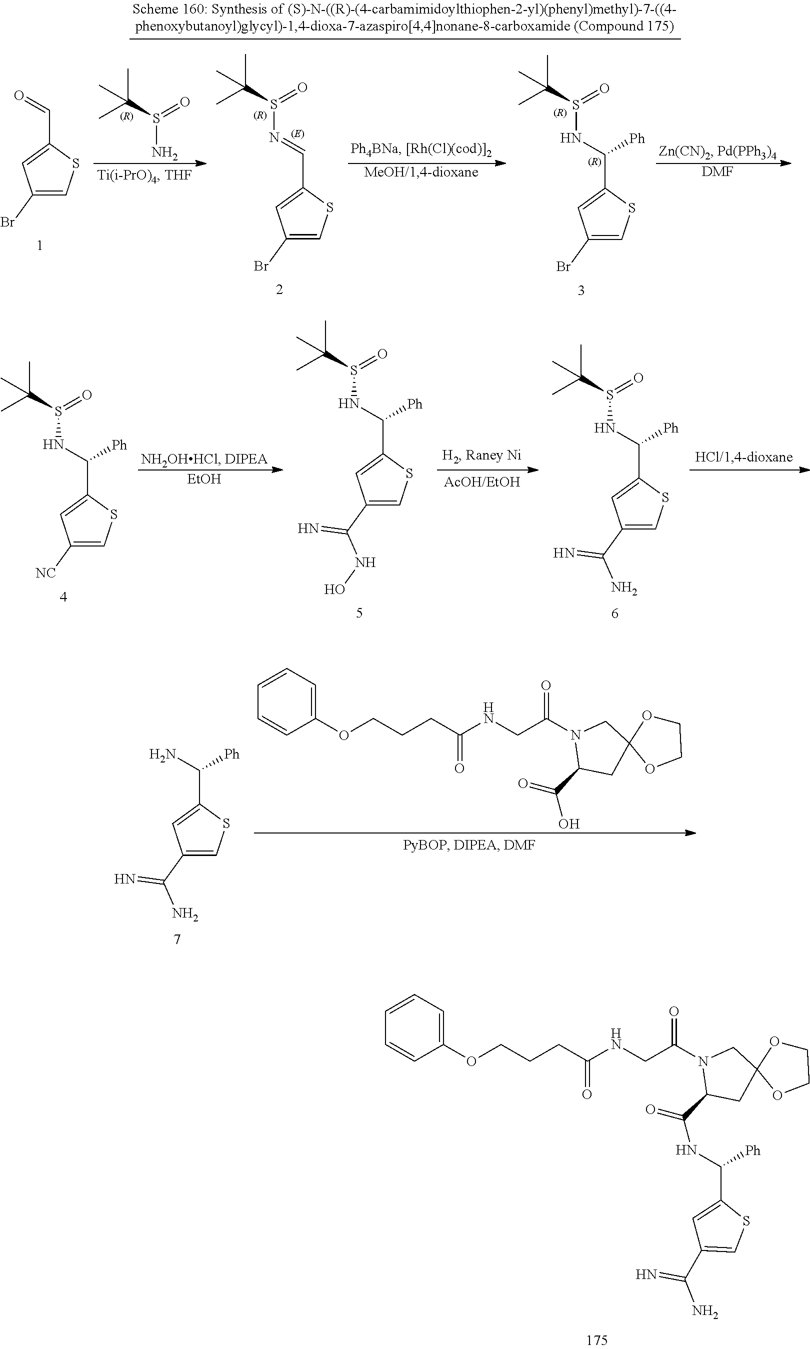

### Step 1: (R,E)-N-((4-bromothiophen-2-yl)methylene)-2-methylpropane-2-sulfinamide (2)

To a solution of 4-bromothiophene-2-carbaldehyde (2 g, 10.47 mmol) and (R)-2-methylpropane-2-sulfinamide (1.4 g, 11.52 mmol) in THE (20 mL) was added Ti(i-PrO)₄ (4.5 g, 15.70 mmol). The mixture was heated to 75° C. under N₂ atmosphere stirred at 75° C. overnight. The mixture was diluted with ethyl acetate and washed with H₂O. The aqueous layer was extracted with ethyl acetate twice and the combined organic layers were washed with brine, dried over anhydrous Na₂SO₄, filtered and concentrated to dryness under reduced pressure. The residue was purified by flash column chromatography on silica gel (petroleum ether:ethyl acetate=1:1) to afford (R,E)-N-((4-bromothiophen-2-yl)methylene)-2-methylpropane-2-sulfinamide (2.8 g, Yield 90.9%) as white solid. LC/MS (ESI) m/z:294 (M+H)⁺.

### Step 2: (R)-N-((R)-(4-bromothiophen-2-yl)(phenyl)methyl)-2-methylpropane-2-sulfinamide (3)

To a solution of (R,E)-N-((4-bromothiophen-2-yl)methylene)-2-methylpropane-2-sulfinamide (1 g, 3.40 mmol) and MeOH (218 mg, 6.80 mmol) in 1,4-dioxane (10 mL) was added Ph₄BNa (1.40 g, 4.08 mmol) and [Rh(Cl)(cod)]₂ (34 mg, 0.068 mmol). The mixture was heated up to 65° C. under N₂ atmosphere and the reaction mixture was stirred overnight. The mixture was diluted with ethyl acetate and washed with H₂O. The aqueous layer was extracted with ethyl acetate twice and the combined organic layers were washed with brine, dried over anhydrous Na₂SO₄, filtered and concentrated to dryness under reduced pressure. The residue was purified by flash column chromatography on silica gel (petroleum ether:ethyl acetate=5:1) to afford (R)-N-((R)-(4-bromothiophen-2-yl)(phenyl)methyl)-2-methylpropane-2-sulfinamide (1.2 g, 3.22 mmol, Yield 94.9%) as white solid. LC/MS (ESI) m/z:372 (M+H)⁺.

### Step 3: N-((R)-(4-cyanothiophen-2-yl)(phenyl)methyl)-2-methylpropane-2-sulfinamide (4)

To a solution of (R)-N-((R)-(4-bromothiophen-2-yl)(phenyl)methyl)-2-methylpropane-2-sulfinamide (600 mg, 1.61 mmol) in NMP (10 mL) was added Zn(CN)₂ (378 mg, 3.22 mmol) and Pd(PPh₃)₄ (186 mg, 0.16 mmol). The mixture was heated up to 160° C. under N₂ atmosphere and stirred overnight.

The mixture was diluted with ethyl acetate and washed with water. The aqueous layer was extracted with ethyl acetate twice and the combined organic layers were washed with brine, dried over anhydrous Na₂SO₄, filtered and concentrated to dryness under reduced pressure. The residue was purified by flash column chromatography on silica gel (petroleum ether:ethyl acetate=5:1) to afford N-((R)-(4-cyanothiophen-2-yl)(phenyl)methyl)-2-methylpropane-2-sulfinamide (80 mg, Yield 15.6%) as white solid. LC/MS (ESI) m/z: 319(M+H)⁺.

### Step 4: 5-((1R)-((tert-butylsulfinyl)amino)(phenyl)methyl)-N-hydroxythiophene-3-carboximidamide (5)

To a solution of N-((R)-(4-cyanothiophen-2-yl)(phenyl)methyl)-2-methylpropane-2-sulfinamide (80 mg, 0.25 mmol) in EtOH (5 mL) was added NH₂OH hydrochloride (44 mg, 0.63 mmol) and DIPEA (98 mg, 0.75 mmol) under N₂ atmosphere and the reaction mixture was stirred at room temperature for 3 hours. The mixture was diluted with ethyl acetate and washed with water. The aqueous layer was extracted with ethyl acetate twice and the combined organic layers were washed with brine, dried over anhydrous Na₂SO₄, filtered and concentrated to dryness under reduced pressure. The residue was purified by flash column chromatography on silica gel (dichloromethane:methanol=10:1) to afford 5-((1R)-((tert-butylsulfinyl)amino)(phenyl) methyl)-N-hydroxythiophene-3-carboximidamide (50 mg, Yield 56.6%) as white solid. LC/MS (ESI) m/z:352 (M+H)⁺.

### Step 5: 5-((1R)-((tert-butylsulfinyl)amino)(phenyl)methyl)thiophene-3-carboximidamide (6)

To a solution of 5-((1R)-((tert-butylsulfinyl)amino)(phenyl)methyl)-N-hydroxythiophene-3-carboximidamide (50 mg, 0.14 mmol) in MeOH (2 mL) was added Raney Nickel (1 mL) and AcOH (0.1 mL) under N₂ atmosphere and the reaction mixture was stirred at room temperature for 3 hours. The mixture was concentrated to dryness under reduced pressure to afford 5-((1R)-((tert-butylsulfinyl)amino)(phenyl)methyl)thiophene-3-carboximidamide (45 mg, Yield 94.1%) as white solid. LC/MS (ESI) m/z:336 (M+H)⁺.

### Step 6: (R)-5-(amino(phenyl)methyl)thiophene-3-carboximidamide (7)

To a solution of 5-((1R)-((tert-butylsulfinyl)amino)(phenyl)methyl)thiophene-3-carboximidamide (45 mg, 0.13 mmol) in 1,4-dioxane (3 mL) was added HCl/1,4-dioxane (1 mL) under N₂ atmosphere and the reaction mixture was stirred at room temperature for 2 hours. The mixture was concentrated to dryness under reduced pressure to afford (R)-5-(amino(phenyl)methyl)thiophene-3-carboximidamide (30 mg, Yield 96.8%) as white solid. LC/MS (ESI) m/z:232 (M+H).

### Step 7: (S)-N-((R)-(4-carbamimidoylthiophen-2-yl)(phenyl)methyl)-7-((4-phenoxybutanoyl) glycyl)-1,4-dioxa-7-azaspiro[4.4]nonane-8-carboxamide (Compound 175)

To a solution of (S)-7-((4-phenoxybutanoyl)glycyl)-1,4-dioxa-7-azaspiro[4.4]nonane-8-carboxylic acid (51 mg, 0.13 mmol) and (R)-5-(amino(phenyl)methyl) thiophene-3-carboximidamide (30 mg, 0.13 mmol) in DMF (5 mL) was added DIPEA (101 mg, 0.78 mmol) and T₃P (124 mg, 0.19 mmol, 50% in ethyl acetate) at 0° C. under N₂ atmosphere and the reaction mixture was stirred at room temperature for 2 hours. The mixture was diluted with ethyl acetate and washed with saturated aq.NaHCO₃ solution. The aqueous layer was extracted with ethyl acetate twice and the combined organic layers were washed with brine, dried over anhydrous Na₂SO₄, filtered and concentrated to dryness under reduced pressure. The residue was purified by prep-HPLC to give Compound 175 (5 mg, Yield 6.4%) as a white solid. ¹H NMR (400 MHz, CD₃OD) δ 8.55 (s, 1H), 8.27 (dd, J=6.9, 1.6 Hz, 1H), 7.48 (dd, J=13.5, 12.1 Hz, 1H), 7.41-7.32 (m, 5H), 7.23 (ddd, J=9.7, 6.8, 2.9 Hz, 2H), 6.91-6.87 (m, 3H), 6.43 (d, J=35.5 Hz, 1H), 4.61 (dd, J=8.7, 6.8 Hz, 1H), 4.04-3.93 (m, 8H), 3.76-3.68 (m, 2H), 2.46 (t, J=7.4 Hz, 2H), 2.38 (dd, J=13.1, 8.8 Hz, 1H), 2.26-2.12 (m, 1H), 2.09-2.02 (m, 2H). LC/MS (ESI) m/z:606 (M+H)⁺.

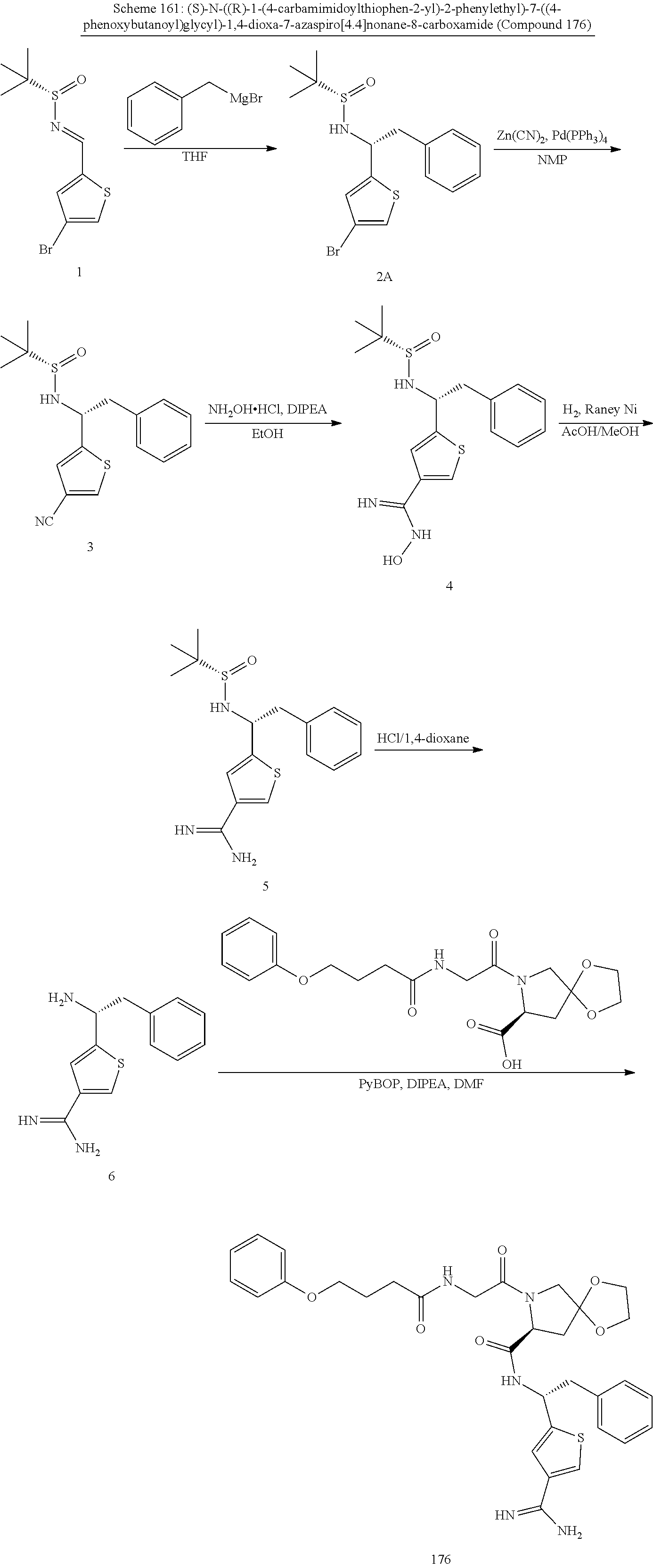

### Step 1: (S)-N-((R)-1-(4-bromothiophen-2-yl)-2-phenylethyl)-2-methylpropane-2-sulfinamide

To a solution of (S,E)-N-((4-bromothiophen-2-yl)methylene)-2-methylpropane-2-sulfinamide (1.0 g, 3.40 mmol) in THE (10 mL) was added benzylmagnesium bromide (5.7 mL, 5.10 mmol) dropwise at −78° C. under N₂ atmosphere and the reaction mixture was stirred at −78° C. for 2 hours. The mixture was diluted with ethyl acetate and washed with saturated aq.NH₄Cl solution. The aqueous layer was extracted with ethyl acetate twice and the combined organic layers were washed with brine, dried over anhydrous Na₂SO₄, filtered and concentrated to dryness under reduced pressure. The residue was purified by flash column chromatography on silica gel (petroleum ether:ethyl acetate=1:1) to give (S)-N-((R)-1-(4-bromothiophen-2-yl)-2-phenylethyl)-2-methylpropane-2-sulfinamide (291 mg, yield 22.4%) as light oil. LC/MS (ESI) m/z: 386/388 (M+H)⁺.

### Step 2: (S)-N-((R)-1-(4-cyanothiophen-2-yl)-2-phenylethyl)-2-methylpropane-2-sulfinamide

To a solution of (S)-N-((R)-1-(4-bromothiophen-2-yl)-2-phenylethyl)-2-methylpropane-2-sulfinamide (240 mg, 0.62 mmol) in NMP (3 mL) was added Zn(CN)₂ (146 mg, 1.25 mmol) and Pd(PPh₃)₄ (72 mg, 0.06 mmol). The reaction mixture was heated up to 160° C. under N₂ atmosphere and stirred for 2 hours. The mixture was diluted with ethyl acetate and washed with water. The aqueous layer was extracted with ethyl acetate twice and the combined organic layers were washed with brine, dried over anhydrous Na₂SO₄, filtered and concentrated to dryness under reduced pressure. The residue was purified by flash column chromatography on silica gel (petroleum ether:ethyl acetate=1:1) to give (S)-N-((R)-1-(4-cyanothiophen-2-yl)-2-phenylethyl)-2-methylpropane-2-sulfinamide (143 mg, yield 69.4%) as light oil. LC/MS (ESI) m/z: 333 (M+H)⁺.

### Step 3: 5-((R)-1-(((S)-tert-butylsulfinyl)amino)-2-phenylethyl)-N-hydroxythiophene-3-carboximidamide

To a solution of (S)-N-((R)-1-(4-cyanothiophen-2-yl)-2-phenylethyl)-2-methylpropane-2-sulfinamide (143 mg, 0.43 mmol) in EtOH (3 mL) was added NH₂OH hydrochloride (75 mg, 1.08 mmol) and DIPEA (0.2 mL, 1.29 mmol) under N₂ atmosphere and the reaction mixture was stirred at room temperature overnight. The mixture was diluted with ethyl acetate and washed with water. The aqueous layer was extracted with ethyl acetate twice and the combined organic layers were washed with brine, dried over anhydrous Na₂SO₄, filtered and concentrated to dryness under reduced pressure. The residue was purified by flash column chromatography on silica gel (petroleum ether:ethyl acetate=1:2) to give 5-((R)-1-(((S)-tert-butylsulfinyl)amino)-2-phenylethyl)-N-hydroxythiophene-3-carboximidamide (95 mg, yield 60.5%) as white solid. LC/MS (ESI) m/z:366 (M+H)⁺.

### Step 4: 5-((R)-1-(((S)-tert-butylsulfinyl)amino)-2-phenylethyl)thiophene-3-carboximidamide

To a solution of 5-((R)-1-(((S)-tert-butylsulfinyl)amino)-2-phenylethyl)-N-hydroxythiophene-3-carboximidamide (95 mg, 0.26 mmol) in methanol (3 mL) was added Raney Nickel (20 mg) and AcOH (one drop) under H₂ atmosphere and the reaction mixture was stirred at room temperature overnight.

The mixture was concentrated to dryness under reduced pressure to give 5-((R)-1-(((S)-tert-butylsulfinyl)amino)-2-phenylethyl)thiophene-3-carboximidamide (83 mg, yield 91.24%) as white solid.

LC/MS (ESI) m/z:350 (M+H)⁺.

### Step 5: (R)-5-(1-amino-2-phenylethyl)thiophene-3-carboximidamide

A solution of 5-((R)-1-(((S)-tert-butylsulfinyl)amino)-2-phenylethyl)thiophene-3-carboximidamide (83 mg, 0.24 mmol) in HCl/1,4-dioxane (2 mL, 4M) was stirred at room temperature under N₂ atmosphere for 1 hour. The mixture was concentrated to dryness under reduced pressure to give (R)-5-(1-amino-2-phenylethyl)thiophene-3-carboximidamide(58 mg, yield 98.3%) as white solid. LC/MS (ESI) m/z:246 (M+H)⁺.

### Step 6: (S)-N-((R)-1-(4-carbamimidoylthiophen-2-yl)-2-phenylethyl)-7-((4-phenoxybutanoyl) glycyl)-1,4-dioxa-7-azaspiro[4.4]nonane-8-carboxamide (Compound 176)

To a solution of (R)-5-(1-amino-2-phenylethyl)thiophene-3-carboximidamide(30 mg, 0.18 mmol) and (S)-7-((4-phenoxybutanoyl)glycyl)-1,4-dioxa-7-azaspiro[4.4]nonane-8-carboxylic acid (30 mg, 0.08 mmol) in DMF (2 mL) was added DIPEA (0.06 mL, 0.40 mmol) and T₃P (61 mg, 0.10 mmol, 50% wt in ethyl acetate) at 0° C. under N₂ atmosphere and the mixture was stirred at room temperature for 1 hour. The mixture was diluted with ethyl acetate and washed with saturated aq.NaHCO₃ solution. The aqueous layer was extracted with ethyl acetate twice and the combined organic layers were washed with brine, dried over anhydrous Na₂SO₄, filtered and concentrated to dryness under reduced pressure. The residue was purified by pre-HPLC to give Compound 176 (1.9 mg, yield 3.9%) as a white solid. ¹H NMR (400 MHz, CD₃OD) δ 8.23 (d, J=1.2 Hz, 1H), 7.51 (d, J=12.4 Hz, 1H), 7.28-7.19 (m, 7H), 6.90-6.85 (m, 3H), 5.44 (dd, J=9.6, 9.6 Hz, 1H), 4.46-4.38 (m, 1H), 4.01-3.83 (m, 9H), 3.67-3.55 (m, 2H), 3.10-3.04 (m, 1H), 2.44 (t, J=7.4 Hz, 2H), 2.07 (dd, J=13.8, 7.4 Hz, 3H), 1.77-1.71 (m, 1H). LC/MS (ESI) m/z:620 (M+H)⁺.

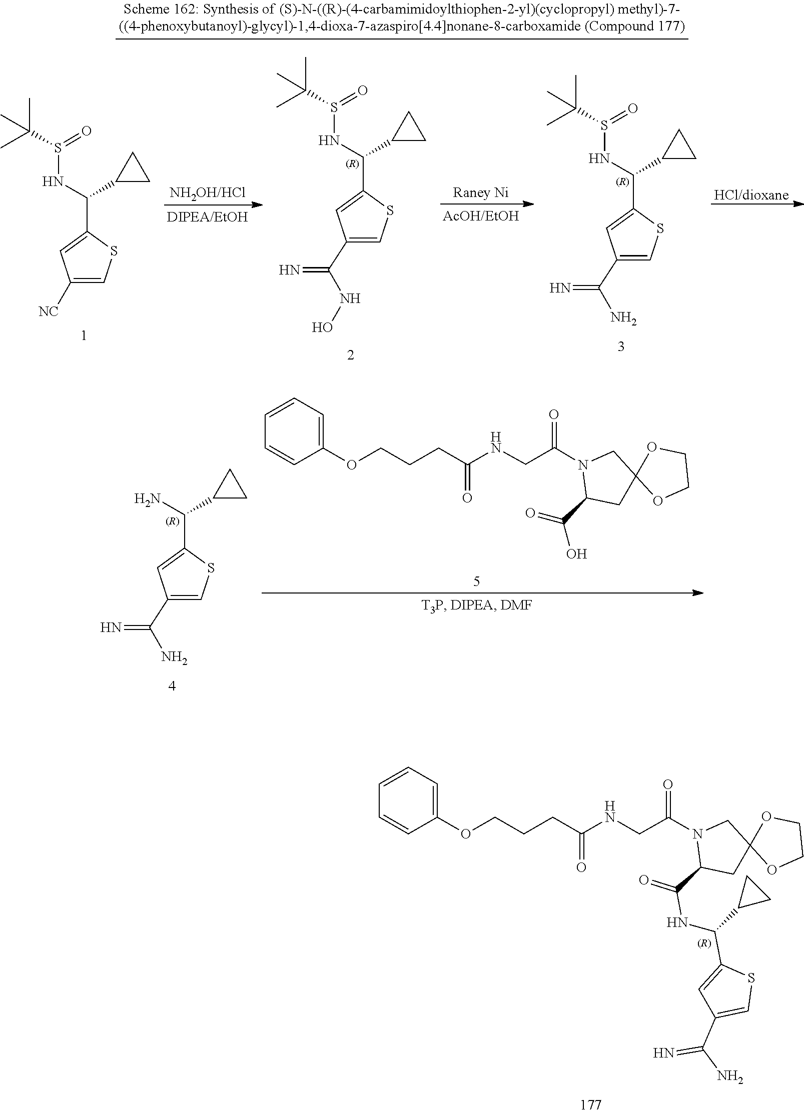

### Step 1: 5-((R)-(((S)-tert-butylsulfinyl)amino)(cyclopropyl)methyl)-N-hydroxythiophene-3-carboximidamide (2)

To a solution of (S)-N-((R)-(4-cyanothiophen-2-yl)(cyclopropyl)methyl)-2-methylpropane-2-sulfinamide (84 mg, 0.30 mmol) in EtOH (3 mL) was added NH₂OH hydrochloride (52 mg, 0.74 mmol) and DIPEA (0.25 mL, 1.50 mmol) under N₂ atmosphere and the reaction mixture was stirred at room temperature overnight. The mixture was diluted with DCM and washed with water. The aqueous layer was extracted with DCM twice and the combined organic layers were washed with brine, dried over anhydrous Na₂SO₄, filtered and concentrated to dryness under reduced pressure. The residue was purified by flash column chromatography on silica gel (dichloromethane:methanol=94:6) to give 5-((R)-(((S)-tert-butylsulfinyl)amino)(cyclopropyl)methyl)-N-hydroxythiophene-3-carboximidamide (90 mg, yield 95.7%) as white solid. LC/MS (ESI) m/z: 316 (M+H)⁺.

### Step 2: 5-((R)-(((S)-tert-butylsulfinyl)amino)(cyclopropyl)methyl)thiophene-3-carboximidamide (5)

To a solution of 5-((R)-(((S)-tert-butylsulfinyl)amino)(cyclopropyl)methyl)-N-hydroxythiophene-3-carboximidamide (90 mg, 0.29 mmol) in methanol (3 mL) was added Raney Nickel (20 mg) and AcOH (one drop) under H₂ atmosphere and the reaction mixture was stirred at room temperature overnight. The mixture was concentrated to dryness under reduced pressure to give 5-((R)-(((S)-tert-butylsulfinyl)amino)(cyclopropyl)methyl)thiophene-3-carboximidamide (75 mg, yield 94.9%) as white solid. LC/MS (ESI) m/z: 300 (M+H)⁺.

### Step 3: (R)-5-(amino(cyclopropyl)methyl)thiophene-3-carboximidamide (4)

To a solution of 5-((R)-(((S)-tert-butylsulfinyl)amino)(cyclopropyl)methyl)thiophene-3-carboximidamide (75 mg, 0.25 mmol) in HCl/1,4-dioxane (3 mL, 4M) was stirred at room temperature under N₂ atmosphere for 1 hour. The mixture was concentrated to dryness under reduced pressure to give (R)-5-(amino(cyclopropyl)methyl)thiophene-3-carboximidamide (45 mg, yield 91.8%) as white solid. LC/MS (ESI) m/z: 196 (M+H)⁺.

### Step 4: (S)-N-((R)-(4-carbamimidoylthiophen-2-yl)(cyclopropyl)methyl)-7-((4-phenoxybutanoyl)glycyl)-1,4-dioxa-7-azaspiro[4.4]nonane-8-carboxamide (Compound 177)

To a solution of (R)-5-(amino(cyclopropyl)methyl)thiophene-3-carboximidamide (30 mg, 0.15 mmol) and (S)-7-((4-phenoxybutanoyl)glycyl)-1,4-dioxa-7-azaspiro[4.4]nonane-8-carboxylic acid (50 mg, 0.13 mmol) in DMF (3 mL) was added DIPEA (0.1 mL, 0.78 mmol) and T₃P (97 mg, 0.15 mmol, 50% wt. in ethyl acetate) at 0° C. under N₂ atmosphere and the mixture was stirred at room temperature for 3 hours. The mixture was diluted with ethyl acetate and washed with saturated aq.NaHCO₃ solution. The aqueous layer was extracted with ethyl acetate twice and the combined organic layers were washed with brine, dried over anhydrous Na₂SO₄, filtered and concentrated to dryness under reduced pressure. The residue was purified by prep-HPLC to give Compound 177 (5 mg, yield 6.9%) as a white solid. ¹H NMR (400 MHz, CD₃OD) δ 8.53 (s, 1H), 8.24 (dd, J=4.9, 1.6 Hz, 1H), 7.59 (t, J=1.4 Hz, 1H), 7.23 (dd, J=9.7, 6.4 Hz, 2H), 6.91-6.87 (m, 3H), 4.67-4.53 (m, 1H), 4.46 (t, J=12.7 Hz, 1H), 4.05-3.95 (m, 8H), 3.71 (q, J=10.5 Hz, 2H), 2.44 (dt, J=12.6, 8.1 Hz, 3H), 2.20 (dd, J=13.1, 6.7 Hz, 1H), 2.08 (dd, J=14.1, 6.7 Hz, 2H), 1.32 (ddd, J=12.6, 8.0, 4.6 Hz, 1H), 0.80-0.72 (m, 1H), 0.63 (ddd, J=13.3, 9.1, 4.4 Hz, 1H), 0.55-0.43 (m, 2H). LC/MS (ESI) m/z: 570 (M+H)⁺.

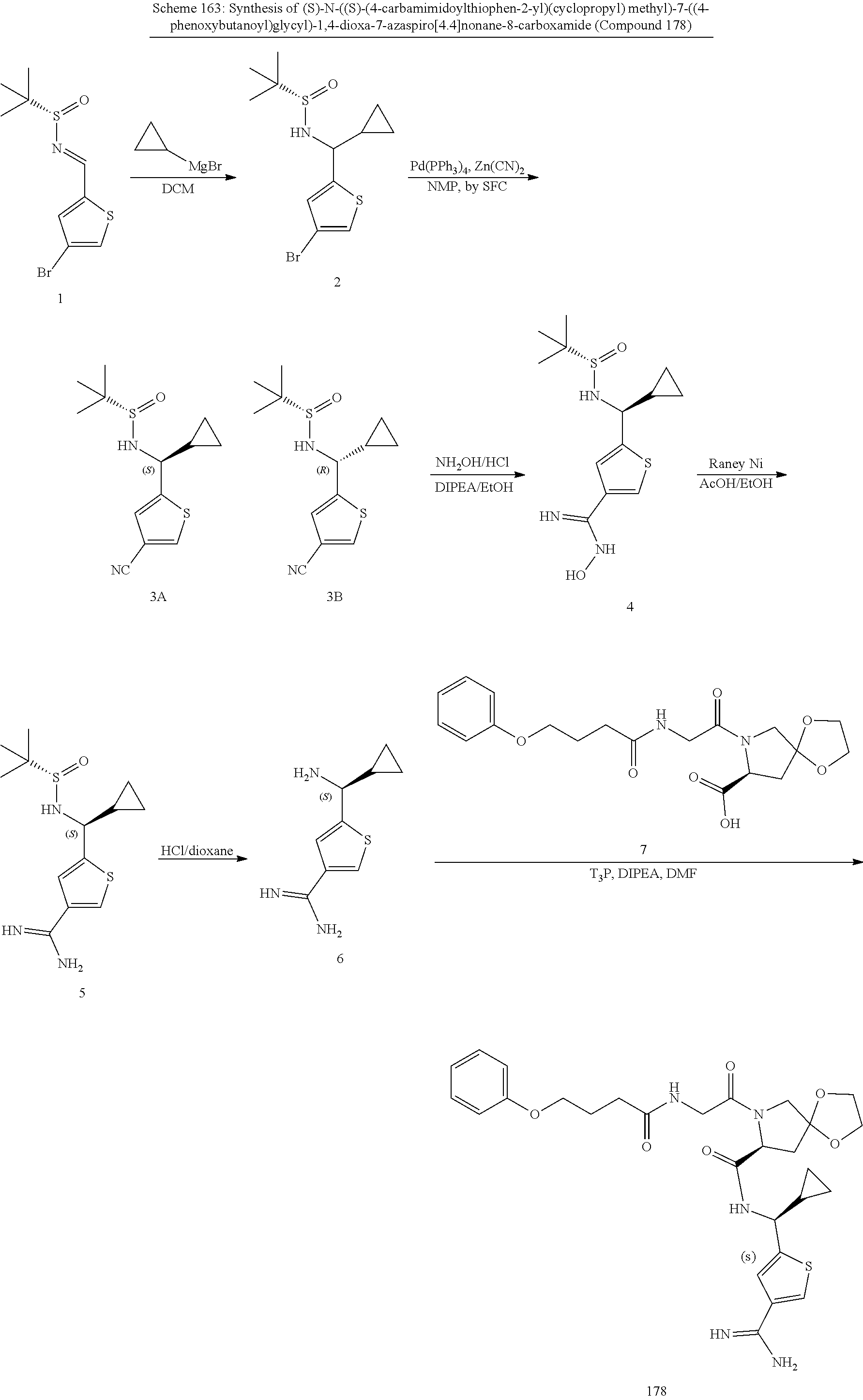

### Step 1: (S)-N-((4-bromothiophen-2-yl)(cyclopropyl)methyl)-2-methylpropane-2-sulfinamide (2)

To a solution of (S,E)-N-((4-bromothiophen-2-yl)methylene)-2-methylpropane-2-sulfinamide (1.0 g, 3.40 mmol) in DCM (15 mL) was added cyclopropyl magnesium bromide (10.2 mL, 10.2 mmol) dropwise at −78° C. under N₂ atmosphere and the reaction mixture was stirred at −78° C. for 2 hours. The mixture was diluted with ethyl acetate and washed with saturated aq. NH₄Cl solution. The aqueous layer was extracted with ethyl acetate twice and the combined organic layers were washed with brine, dried over anhydrous Na₂SO₄, filtered and concentrated to dryness under reduced pressure. The residue was purified by flash column chromatography on silica gel (petroleum ether:ethyl acetate=1:1) to give (S)-N-((4-bromothiophen-2-yl)(cyclopropyl)methyl)-2-methylpropane-2-sulfinamide (1.05 g, yield 92.1%) as light oil. LC/MS (ESI) m/z: 336/338 (M+H)⁺.

### Step 2: (S)-N-((S)-(4-cyanothiophen-2-yl)(cyclopropyl)methyl)-2-methylpropane-2-sulfinamide(3A) and (S)-N-((R)-(4-cyanothiophen-2-yl)(cyclopropyl)methyl)-2-methylpropane-2-sulfinamide(3B)

To a solution of (S)-N-((4-bromothiophen-2-yl)(cyclopropyl)methyl)-2-methylpropane-2-sulfinamide (1.1 g, 3.28 mmol) in NMP (11 mL) was added Zn(CN)₂ (1.16 g, 9.85 mmol) and Pd(PPh₃)₄ (759 mg, 0.66 mmol). The reaction mixture was heated to 100° C. under N₂ atmosphere and the reaction mixture was stirred at 100° C. for 16 hours. The mixture was diluted with ethyl acetate and washed with water. The aqueous layer was extracted with ethyl acetate twice and the combined organic layers were washed with brine, dried over anhydrous Na₂SO₄, filtered and concentrated to dryness under reduced pressure. The residue was purified by flash column chromatography on silica gel (petroleum ether:ethyl acetate=3:2) to give the crude product which was further purified by SFC to give (S)-N-((S)-(4-cyanothiophen-2-yl)(cyclopropyl)methyl)-2-methylpropane-2-sulfinamide (140 mg, yield 56.0%) and (S)-N-((R)-(4-cyanothiophen-2-yl)(cyclopropyl)methyl)-2-methylpropane-2-sulfinamide (85 mg, yield 34.0%) as light oil. LC/MS (ESI) m/z: 283 (M+H)⁺.

### Step 3: 5-((S)-(((S)-tert-butylsulfinyl)amino)(cyclopropyl)methyl)-N-hydroxythiophene-3-carboximidamide(4)

To a solution of (S)-N-((S)-(4-cyanothiophen-2-yl)(cyclopropyl)methyl)-2-methylpropane-2-sulfinamide (110 mg, 0.39 mmol) in EtOH (3 mL) was added NH₂OH hydrochloride (68 mg, 0.98 mmol) and DIPEA (0.2 mL, 1.17 mmol) under N₂ atmosphere and the reaction mixture was stirred at room temperature overnight. The mixture was diluted with dichloromethane and washed with water. The aqueous layer was extracted with dichloromethane twice and the combined organic layers were washed with brine, dried over anhydrous Na₂SO₄, filtered and concentrated to dryness under reduced pressure. The residue was purified by flash column chromatography on silica gel (dichloromethane:methanol=94:6) to give 5-((S)-(((S)-tert-butylsulfinyl)amino)(cyclopropyl)methyl)-N-hydroxythiophene-3-carboximidamide (120 mg, yield 98.4%) as white solid. LC/MS (ESI) m/z: 316 (M+H)⁺.

### Step 4: (S)-N-((4-carbamimidoylthiophen-2-yl)(cyclopropyl)methyl)pivalamide(5)

To a solution of 5-((S)-(((S)-tert-butylsulfinyl)amino)(cyclopropyl)methyl)-N-hydroxythiophene-3-carboximidamide (120 mg, 0.38 mmol) in methanol (3 mL) was added Raney Nickel (20 mg) and AcOH (one drop) under H₂ atmosphere and the reaction mixture was stirred at room temperature overnight. The mixture was concentrated to dryness under reduced pressure to give (S)-N-((4-carbamimidoylthiophen-2-yl)(cyclopropyl)methyl)pivalamide (100 mg, yield 87.7%) as white solid.

LC/MS (ESI) m/z: 300 (M+H)⁺.

### Step 5: (S)-5-(amino(cyclopropyl)methyl)thiophene-3-carboximidamide (6)

To a solution of (S)-N-((4-carbamimidoylthiophen-2-yl)(cyclopropyl)methyl)pivalamide (100 mg, 0.33 mmol) in HCl/1,4-dioxane (3 mL, 4M) was stirred at room temperature under N₂ atmosphere for 1 hour. The mixture was concentrated to dryness under reduced pressure to give (S)-5-(amino(cyclopropyl)methyl)thiophene-3-carboximidamide (60 mg, yield 85.7%) as white solid. LC/MS (ESI) m/z: 196 (M+H)⁺.

### Step 6: (S)-N-((S)-(4-carbamimidoylthiophen-2-yl)(cyclopropyl)methyl)-7-((4-phenoxybutanoyl)glycyl)-1,4-dioxa-7-azaspiro[4.4]nonane-8-carboxamide (Compound 178)

To a solution of (S)-5-(amino(cyclopropyl)methyl)thiophene-3-carboximidamide (30 mg, 0.15 mmol) and (S)-7-((4-phenoxybutanoyl)glycyl)-1,4-dioxa-7-azaspiro[4.4]nonane-8-carboxylic acid (50 mg, 0.13 mmol) in DMF (3 mL) was added DIPEA (0.1 mL, 0.78 mmol) and T₃P (97 mg, 0.15 mmol, 50% wt in ethyl acetate) at 0° C. under N₂ atmosphere and the mixture was stirred at room temperature for 3 hours. The mixture was diluted with ethyl acetate and washed with saturated aq. NaHCO₃ solution. The aqueous layer was extracted with ethyl acetate twice and the combined organic layers were washed with brine, dried over anhydrous Na₂SO₄, filtered and concentrated to dryness under reduced pressure. The residue was purified by prep-HPLC to give Compound 178 (8 mg, yield 10.9%) as a white solid. ¹H NMR (400 MHz, CD₃OD) δ 8.53 (s, 1H), 8.24 (dd, J=4.9, 1.6 Hz, 1H), 7.59 (t, J=1.4 Hz, 1H), 7.23 (dd, J=9.7, 6.4 Hz, 2H), 6.91-6.87 (m, 3H), 4.67-4.53 (m, 1H), 4.46 (t, J=12.7 Hz, 1H), 4.05-3.95 (m, 8H), 3.71 (q, J=10.5 Hz, 2H), 2.44 (dt, J=12.6, 8.1 Hz, 3H), 2.20 (dd, J=13.1, 6.7 Hz, 1H), 2.08 (dd, J=14.1, 6.7 Hz, 2H), 1.32 (ddd, J=12.6, 8.0, 4.6 Hz, 1H), 0.80-0.72 (m, 1H), 0.63 (ddd, J=13.3, 9.1, 4.4 Hz, 1H), 0.55-0.43 (m, 2H). LC/MS (ESI) m/z: 570 (M+H)⁺.

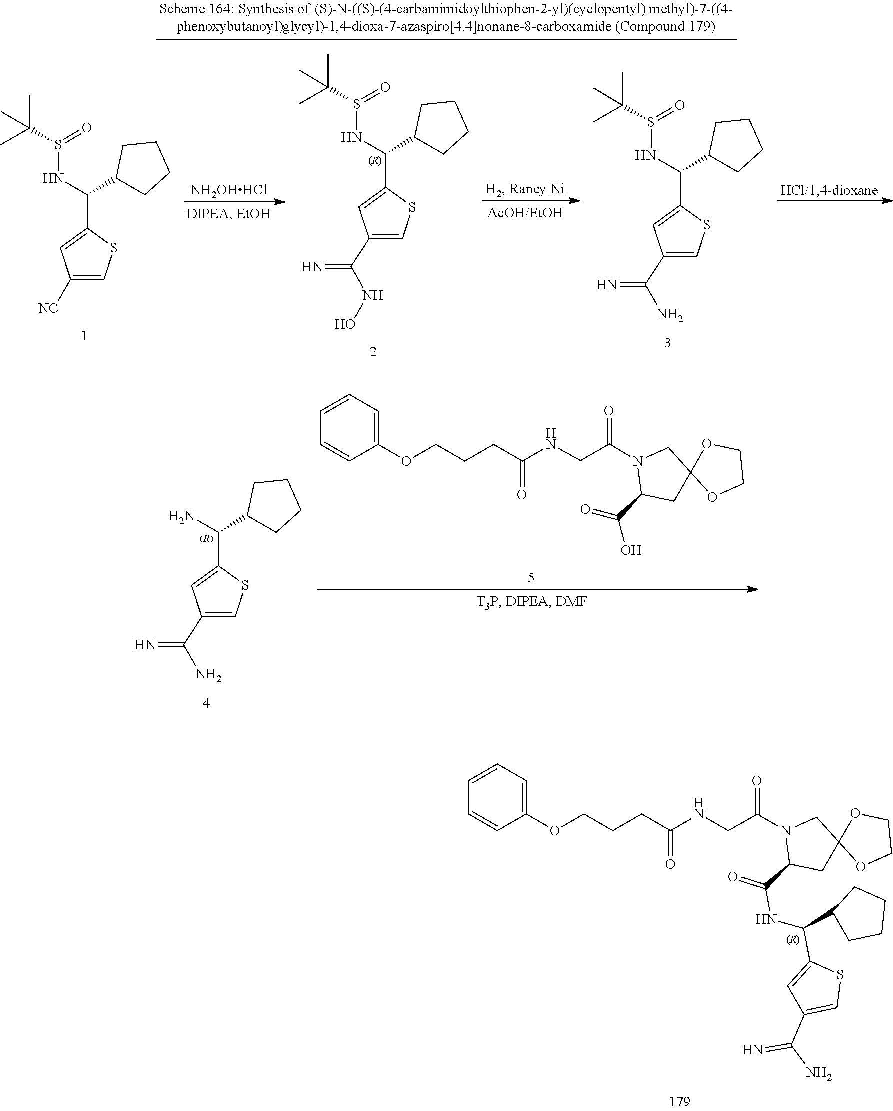

### Step 1: 5-((S)-(((S)-tert-butylsulfinyl)amino)(cyclopentyl)methyl)-N-hydroxythiophene-3-carboximidamide

To a solution of (S)-N-((S)-(4-cyanothiophen-2-yl)(cyclopentyl)methyl)-2-methylpropane-2-sulfinamide (120 mg, 0.39 mmol) in EtOH (3 mL) was added NH₂OH hydrochloride (67 mg, 0.97 mmol) and DIPEA (151 mg, 1.17 mmol) under N₂ atmosphere and the reaction mixture was stirred at room temperature overnight. The mixture was diluted with dichloromethane and washed with water. The aqueous layer was extracted with dichloromethane twice and the combined organic layers were washed with brine, dried over anhydrous Na₂SO₄, filtered and concentrated to dryness under reduced pressure. The residue was purified by flash column chromatography on silica gel (dichloromethane:methanol=100:1) to give 5-((S)-(((S)-tert-butylsulfinyl)amino)(cyclopentyl)methyl)-N-hydroxythiophene-3-carboximidamide (100 mg, yield 75.8%) as white solid. LC/MS (ESI) m/z:344 (M+H)⁺.

### Step 2: 5-((S)-(((S)-tert-butylsulfinyl)amino)(cyclopentyl)methyl)thiophene-3-carboximidamide

To a solution of 5-((S)-(((S)-tert-butylsulfinyl)amino)(cyclopentyl)methyl)-N-hydroxythiophene-3-carboximidamide (100 mg, 0.29 mmol) in methanol (3 mL) was added Raney Nickel (20 mg) and AcOH (one drop) under H₂ atmosphere and the reaction mixture was stirred at room temperature overnight. The mixture was concentrated to dryness under reduced pressure to give 5-((S)-(((S)-tert-butylsulfinyl)amino) (cyclopentyl)methyl)thiophene-3-carboximidamide (70 mg, yield 73.7%) as white solid. LC/MS (ESI) m/z:328 (M+H)⁺.

### Step 3: (S)-5-(amino(cyclopentyl)methyl)thiophene-3-carboximidamide

A solution of 5-((S)-(((S)-tert-butylsulfinyl)amino)(cyclopentyl)methyl)thiophene-3-carboximidamide (70 mg, 0.21 mmol) in HCl/1,4-dioxane (2 mL, 4M) was stirred at room temperature under N₂ atmosphere for 1 hour. The mixture was concentrated to dryness under reduced pressure to give (S)-5-(amino(cyclopentyl)methyl) thiophene-3-carboximidamide hydrochloride (45 mg, yield 94.3%) as white solid. LC/MS (ESI) m/z:224 (M+H)⁺.

### Step 4: (S)-N-((S)-(4-carbamimidoylthiophen-2-yl)(cyclopentyl)methyl)-7-((4-phenoxybutanoyl)glycyl)-1,4-dioxa-7-azaspiro[4.4]nonane-8-carboxamide (Compound 179)

To a solution of (S)-5-(amino(cyclopentyl)methyl)thiophene-3-carboximidamide (40 mg, 0.18 mmol) and (S)-7-((4-phenoxybutanoyl)glycyl)-1,4-dioxa-7-azaspiro[4.4]nonane-8-carboxylic acid (70 mg, 0.18 mmol) in DMF (2 mL) was added DIPEA (116 mg, 0.90 mmol) and T₃P (172 mg, 0.27 mmol, 50% wt. in ethyl acetate) at 0° C. under N₂ atmosphere and the mixture was stirred at room temperature for 1 hour. The mixture was diluted with ethyl acetate and washed with saturated aq.NaHCO₃ solution. The aqueous layer was extracted with ethyl acetate twice and the combined organic layers were washed with brine, dried over anhydrous Na₂SO₄, filtered and concentrated to dryness under reduced pressure. The residue was purified by prep-HPLC to give Compound 179 (2.0 mg, yield 1.9%) as a white solid. ¹H NMR (400 MHz, CD₃OD) δ 8.21-8.16 (m, 1H), 7.43-7.36 (m, 1H), 7.25-7.21 (m, 2H), 6.90-6.87 (m, 3H), 4.91 (d, J=10.0 Hz, 1H), 4.52 (dd, J=9.2, 9.2 Hz, 1H), 4.02-3.92 (m, 8H), 3.73-3.69 (m, 2H), 2.48-2.43 (m, 3H), 2.39-2.32 (m, 1H), 2.14-2.04 (m, 3H), 1.99-1.93 (m, 1H), 1.69-1.57 (m, 5H), 1.50-1.42 (m, 1H), 1.30-1.23 (m, 1H). LC/MS (ESI) m/z:598 (M+H)⁺.

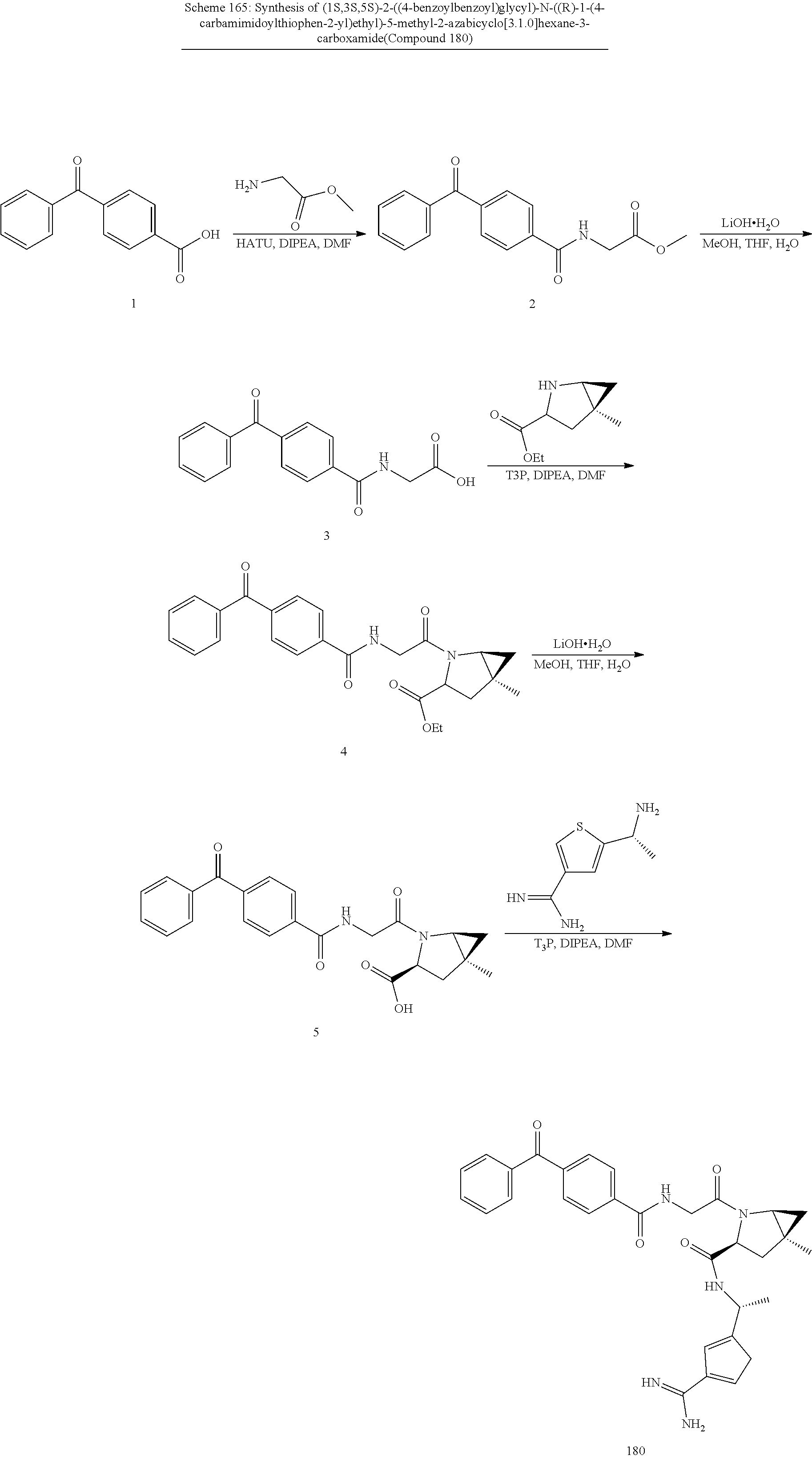

### Step 1: methyl (4-benzoylbenzoyl)glycinate

To a mixture of 4-benzoylbenzoic acid (500 mg, 2.21 mmol) and methyl glycinate hydrochloride (304 mg, 2.4 mmol) in DMF (5 mL) was added DIPEA (1.4 g, 11.05 mmol) and HATU (1.26 g, 3.3 mmol) at 0° C. under N₂ atmosphere and the reaction mixture was stirred at room temperature for 2 hours. The mixture was diluted with water and extracted with ethyl acetate twice. The combined organic layers were washed with brine, dried over anhydrous Na₂SO₄, filtered and concentrated to dryness under reduced pressure. The residue was purified by flash column chromatography on silica gel (petroleum ether:ethyl acetate=1:1) to give methyl (4-benzoylbenzoyl)glycinate (650 mg, yield 98%) as white solid. LC/MS (ESI) m/z:298 (M+H)+.

### Step 2: (4-benzoylbenzoyl)glycine

To a solution of methyl (4-benzoylbenzoyl)glycinate (650 mg, 2.19 mmol) in methanol (6 mL), THE (3 mL) and water (3 mL) was added LiOH·H₂O (277 mg, 6.59 mmol) at 0° C. and the mixture was stirred under N₂ atmosphere at room temperature for 2 hours. The mixture was acidified with 1N aq.HCl solution to pH-3 and extracted with ethyl acetate twice. The combined organic layers were washed with brine, dried over anhydrous Na₂SO₄, filtered and concentrated to dryness under reduced pressure to give (4-benzoylbenzoyl)glycine (541 mg, yield 86.7%) as white solid, which was used directly in next step without further purification. LC/MS (ESI) (m/z): 284 (M+H)+.

### Step 3: ethyl (1S,5S)-2-((4-benzoylbenzoyl)glycyl)-5-methyl-2-azabicyclo[3.1.0]hexane-3-carboxylate

To a mixture of (4-benzoylbenzoyl)glycin (116 mg, 0.407 mmol) and ethyl (1S,3S,5S)-5-methyl-2-azabicyclo[3.1.0]hexane-3-carboxylate hydrochloride(63 mg, 0.37 mmol) in DMF (3 mL) was added DIPEA (286 mg, 2.22 mmol) and T₃P (353 mg, 1.11 mmol, 50% wt. in ethyl acetate) at 0° C. under N₂ atmosphere and the reaction mixture was stirred at room temperature for 1 hour. The mixture was diluted with water and extracted with ethyl acetate twice. The combined organic layers were washed with brine, dried over anhydrous Na₂SO₄, filtered and concentrated to dryness under reduced pressure. The residue was purified by flash column chromatography on silica gel (petroleum ether:ethyl acetate=1:1) to give ethyl (1S,5S)-2-((4-benzoylbenzoyl)glycyl)-5-methyl-2-azabicyclo[3.1.0]hexane-3-carboxylate (150 mg, yield 92.7%) as the colorless oil. LC/MS (ESI) m/z:435 (M+H)+.

### Step 4: (1S,5S)-2-((4-benzoylbenzoyl)glycyl)-5-methyl-2-azabicyclo[3.1.0]hexane-3-carboxylic acid

To a solution of ethyl (1 S,5S)-2-((4-benzoylbenzoyl)glycyl)-5-methyl-2-azabicyclo[3.1.0]hexane-3-carboxylate (150 mg, 0.35 mmol) in methanol (2 mL), THE (1 mL) and water (1 mL) was added LiOH·H₂O (44 mg, 1.05 mmol) at 0° C. and the mixture was stirred under N₂ atmosphere at room temperature for 1.5 hours. The mixture was acidified with 1N aq.HCl solution to pH-3 and extracted with ethyl acetate twice. The combined organic layers were washed with brine, dried over anhydrous Na₂SO₄, filtered and concentrated to dryness under reduced pressure to give (1 S,5S)-2-((4-benzoylbenzoyl)glycyl)-5-methyl-2-azabicyclo[3.1.0]hexane-3-carboxylic acid (121 mg, yield 86.4%) as white solid, which was used directly in next step without further purification. LC/MS (ESI) (m/z): 407 (M+H)⁺.

### Step 5: (1S,5S)-2-((4-benzoylbenzoyl)glycyl)-N-((R)-1-(4-carbamimidoylthiophen-2-yl) ethyl)-5-methyl-2-azabicyclo[3.1.0]hexane-3-carboxamide (Compound 180)

To a mixture of (1 S,5S)-2-((4-benzoylbenzoyl)glycyl)-5-methyl-2-azabicyclo[3.1.0]hexane-3-carboxylic acid (40 mg, 0.1 mmol) and (R)-5-(1-aminoethyl)thiophene-3-carboximidamide (20 mg, 0.11 mmol) in DMF (2 mL) was added DIPEA (65 mg, 0.5 mmol) and T₃P (77 mg, 0.12 mmol, 50% wt. in ethyl acetate) at 0° C. under N₂ atmosphere and the reaction mixture was stirred at room temperature for 2 hours. The mixture was diluted with water and extracted with ethyl acetate twice. The combined organic layers were washed with brine, dried over anhydrous Na₂SO₄, filtered and concentrated to dryness under reduced pressure. The residue was purified by prep-TLC (dichloromethane:methanol=10:1) and prep-HPLC to give Compound 180 (1.0 mg, yield 1.8%) as a white solid. ¹H NMR (400 MHz, CD₃OD) δ 8.55 (s, 1H), 8.21 (d, J=1.6 Hz, 1H), 7.99 (d, J=8.4 Hz, 2H), 7.86-7.79 (m, 4H), 7.68 (t, J=7.5 Hz, 1H), 7.56 (d, J=7.9 Hz, 2H), 7.50 (s, 1H), 5.22 (d, J=6.8 Hz, 1H), 4.82 (s, 1H), 4.46 (d, J=8.2 Hz, 1H), 4.30 (d, J=20.8 Hz, 1H), 3.42-3.39 (m, 1H), 2.44 (t, J=12.4 Hz, 1H), 2.16 (dd, J=3.6, 13.6 Hz, 1H), 1.58 (d, J=6.0 Hz, 3H), 1.31 (s, 3H), 1.22 (dd, J=2.8, 5.6 Hz, 1H), 0.79 (t, J=6.1 Hz, 1H). LC/MS (ESI) (m/z): 558 (M+H)⁺.

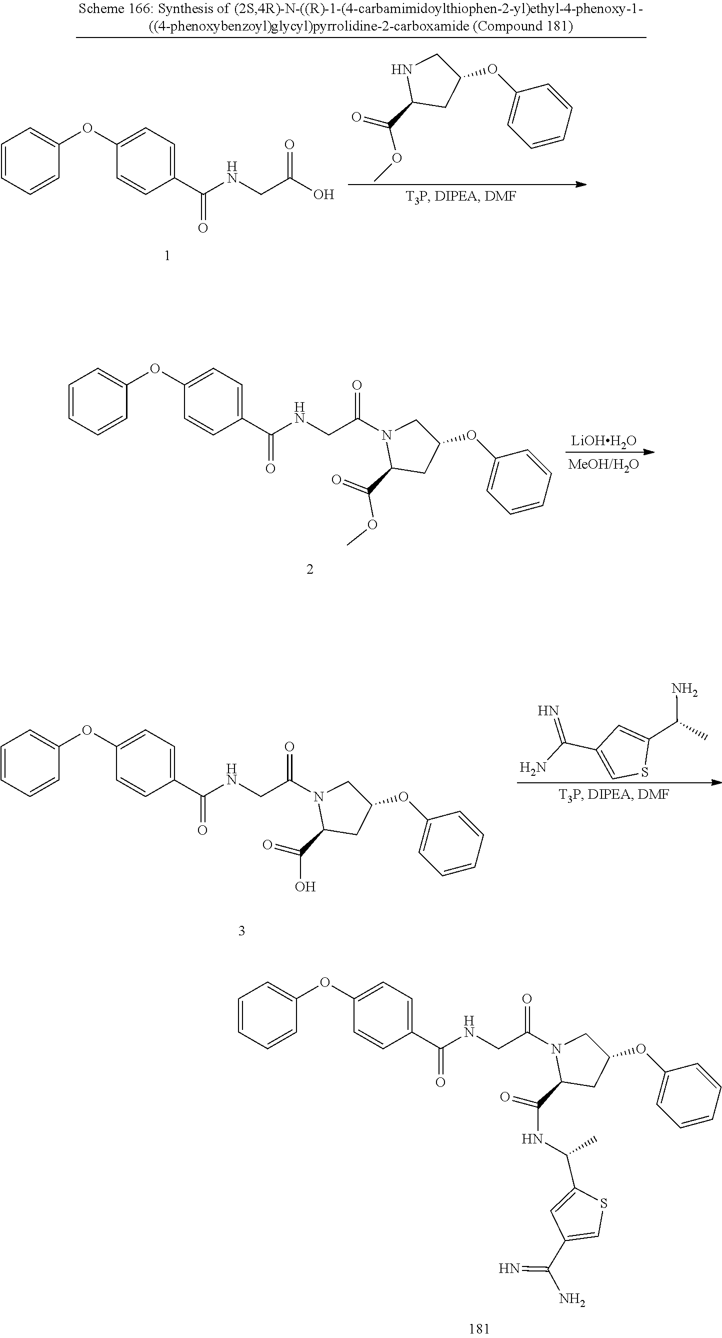

### Step 1: methyl (2S,4R)-4-phenoxy-1-((4-phenoxybenzoyl)glycyl)pyrrolidine-2-carboxylate (2)

To a mixture of benzyl (4-phenoxybenzoyl)glycine (150 mg, 0.55 mmol) and methyl (2S,4R)-4-phenoxypyrrolidine-2-carboxylate hydrochloride (122 mg, 0.55 mmol) in DMF (3.0 mL) was added DIPEA (0.55 mL, 3.30 mmol) and T₃P (528 mg, 0.82 mmol, 50% wt. in ethyl acetate) at 0° C. under N₂ atmosphere and the mixture was stirred at 25° C. for 16 hours. The mixture was diluted with water and extracted with ethyl acetate twice. The combined organic layers were dried over anhydrous Na₂SO₄, filtered and concentrated to dryness under reduced pressure. The residue was purified by flash column chromatography on silica gel (dichloromethane:methanol=98:2) to give methyl (2S,4R)-4-phenoxy-1-((4-phenoxybenzoyl)glycyl)pyrrolidine-2-carboxylate (214 mg, yield 81.7%) as yellow solid. LC/MS (ESI) m/z: 475 (M+H)⁺.

### Step 2: (2S,4R)-4-phenoxy-1-((4-phenoxybenzoyl)glycyl)pyrrolidine-2-carboxylic acid (3)

To a solution of methyl (2S,4R)-4-phenoxy-1-((4-phenoxybenzoyl)glycyl)pyrrolidine-2-carboxylate (214 mg, 0.45 mmol) in methanol (3 mL) and water (0.6 mL) was added lithium hydroxide (28 mg, 0.68 mmol) at 0° C. and the mixture was stirred at room temperature for 2 hours. The mixture was acidified with 1N aq.HCl solution to pH-3, extracted with CHCl₃/i-PrOH(3/1, v/v) five times. The combined organic layers were washed with brine, dried over anhydrous Na₂SO₄, filtered and concentrated to dryness under reduced pressure to give (2S,4R)-4-phenoxy-1-((4-phenoxybenzoyl)glycyl)pyrrolidine-2-carboxylic acid (200 mg, yield 96.1%) as colorless oil, which was used directly in the next step. LC/MS (ESI) (m/z): 461 (M+H)⁺.

### Step 3: (2S,4R)-N-((R)-1-(4-carbamimidoylthiophen-2-yl)ethyl)-4-phenoxy-1-((4-phenoxybenzoyl)glycyl)pyrrolidine-2-carboxamide (Compound 181)

To a mixture of (2S,4R)-4-phenoxy-1-((4-phenoxybenzoyl)glycyl)pyrrolidine-2-carboxylic acid (60 mg, 0.13 mmol) and (R)-5-(1-(I2-azanyl)ethyl)thiophene-3-carboximidamide hydrochloride (33 mg, 0.20 mmol) in DMF (3.0 mL) was added DIPEA (0.11 mL, 0.45 mmol) and T₃P (99 mg, 0.16 mmol, 50% wt in ethyl acetate) at 0° C. under N₂ atmosphere and the mixture was stirred at 25° C. for 16 hours. The mixture was concentrated to dryness under reduced pressure. The residue was purified by prep-TLC (dichloromethane:methanol=10:1) and further purified by prep-HPLC to give Compound 181 (5 mg, yield 6.25%) as a white solid. ¹H NMR (400 MHz, CD₃OD) δ 8.52 (s, 1H), 8.23 (dd, J=10.1, 1.6 Hz, 1H), 7.86-7.82 (m, 2H), 7.55 (dd, J=4.7, 3.4 Hz, 1H), 7.44-7.39 (m, 2H), 7.34-7.28 (m, 2H), 7.23-7.18 (m, 1H), 7.06 (dd, J=8.6, 1.0 Hz, 2H), 7.02-6.95 (m, 5H), 5.27 (q, J=7.1 Hz, 1H), 5.17 (s, 1H), 4.62 (t, J=8.2 Hz, 1H), 4.25 (d, J=16.6 Hz, 1H), 4.18-4.11 (m, 1H), 4.04-3.89 (m, 2H), 2.57 (dd, J=13.9, 8.0 Hz, 1H), 2.26 (ddd, J=13.5, 8.8, 4.6 Hz, 1H), 1.61 (dd, J=30.7, 7.0 Hz, 3H). LC/MS (ESI) m/z: 612 (M+H)⁺.

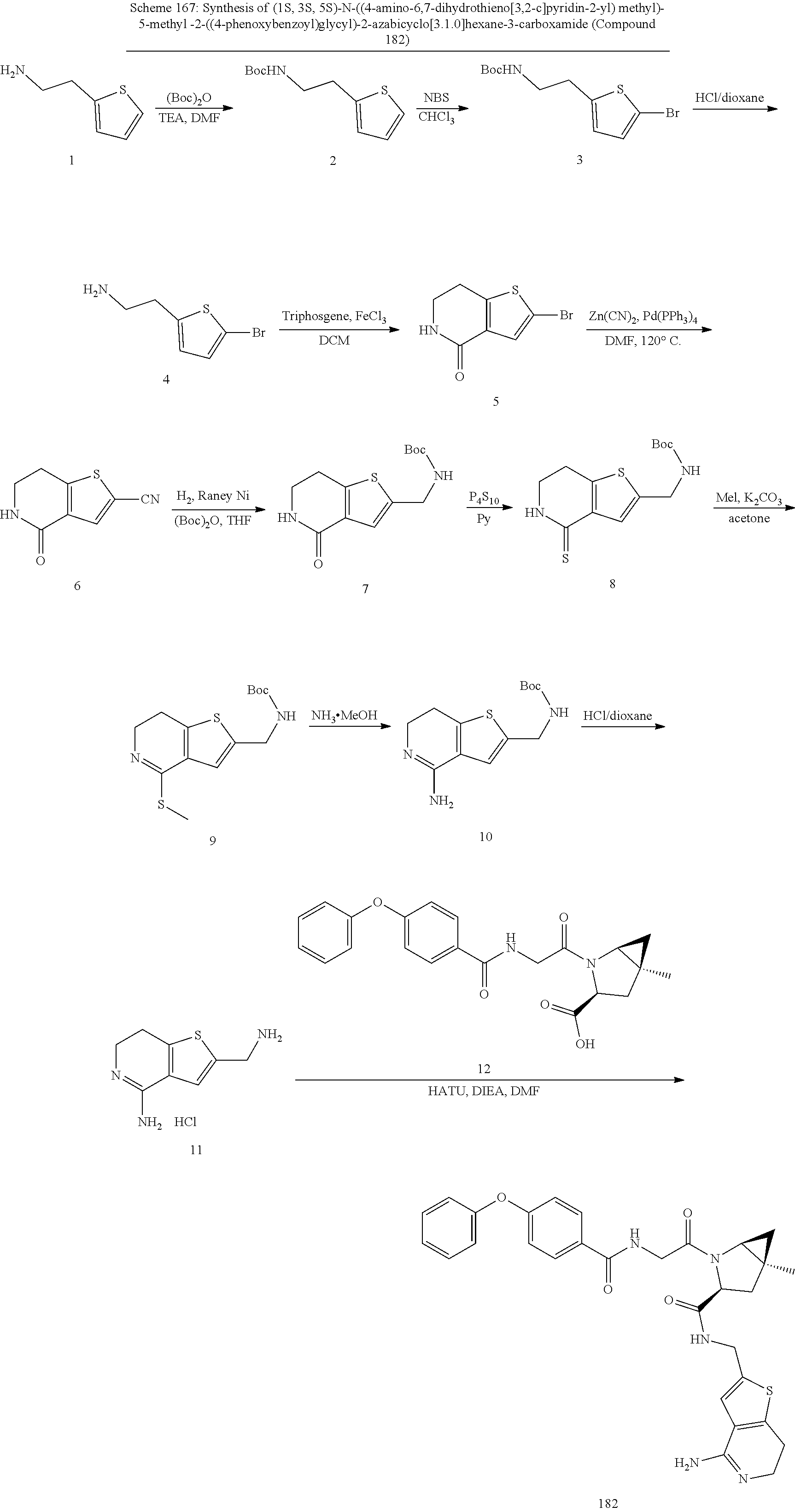

### Step 1: tert-butyl (2-(thiophen-2-yl)ethyl)carbamate (2)

To a solution of 2-(thiophen-2-yl)ethan-1-amine (15 g, 118 mmol) in DCM (200 mL) was added TEA (19 g, 147 mmol) and di-tert-butyl dicarbonate (32 g, 146.7 mmol) and the mixture was stirred at room temperature for 12 hours. The mixture was diluted with water and extracted with dichloromethane twice. The combined organic layers were dried over anhydrous Na₂SO₄, filtered and concentrated to dryness under reduced pressure. The residue was purified by flash column chromatography on silica gel (petroleum ether:ethyl acetate=8:1) to give tert-butyl (2-(thiophen-2-yl)ethyl)carbamate (25 g, yield 93.2%) as colorless oil. LC/MS (ESI) m/z: 172 (M+H−56)⁺.

### Step 2: tert-butyl (2-(5-bromothiophen-2-yl)ethyl)carbamate (3)

To a mixture of tert-butyl (2-(thiophen-2-yl)ethyl)carbamate (20 g, 88.1 mmol) in CHCl₃ (200 mL) was added NBS (17 g, 95.5 mmol) in portions at 0° C. and the mixture was stirred at room temperature for 2 hours. The mixture was diluted with water and extracted with ethyl acetate twice. The combined organic layers were washed with brine, dried over anhydrous Na₂SO₄, filtered and concentrated to dryness under reduced pressure. The residue was purified by flash column chromatography on silica gel (petroleum ether:ethyl acetate=10:1) to give tert-butyl (2-(5-bromothiophen-2-yl)ethyl)carbamate (15 g, yield 55.7%) as a light yellow oil. LC/MS (ESI) m/z: 250 (M+H−56)⁺.

### Step 3: 2-(5-bromothiophen-2-yl)ethan-1-amine hydrochloride (4)

To a solution of tert-butyl (2-(5-bromothiophen-2-yl)ethyl)carbamate (10 g, 32.6 mmol) in DCM (10 mL) was added HCl/1,4-dioxane (30 mL, 4 M) at 0° C. and the mixture was stirred at room temperature for 2 hours. The mixture was concentrated to dryness under reduced pressure and the resulting residue was neutralized with saturated aq.NaHCO₃ solution and extracted with DCM twice.

The combined organic layers were washed with brine, dried over anhydrous Na₂SO₄, filtered and concentrated to dryness under reduced pressure to give 2-(5-bromothiophen-2-yl)ethan-1-amine hydrochloride (6 g, yield 89%) as white solid. LC/MS (ESI) m/z: 205 (M+H)⁺.

### Step 4: 2-bromo-6,7-dihydrothieno[3,2-c]pyridin-4(5H)-one (5)

To a solution of 2-(5-bromothiophen-2-yl)ethan-1-amine (4 g, 19.5 mmol) in DCM (60 mL) was added triphosgene (1.9 g, 6.44 mmol) at 0° C. and the mixture was stirred for 0.5 hour. The mixture was diluted with saturated aq.NaHCO₃ solution and extracted with DCM twice. The combined organic layers were dried over anhydrous Na₂SO₄, filtered and concentrated to dryness under reduced pressure. The residue was dissolved in DCM (20 mL) and FeCl₃ (6.31 g, 39.0 mmol) was added and the resulting mixture was stirred at 50° C. for 2 hours. The mixture was diluted with water and extracted with DCM twice and the combined organic layers were dried over anhydrous Na₂SO₄, filtered and concentrated to dryness under reduced pressure. The residue was purified by flash column chromatography on silica gel (petroleum ether:ethyl acetate=1:1) to give 2-bromo-6,7-dihydrothieno[3,2-c]pyridin-4(5H)-one (1 g, yield 22%) as white solid. LC/MS (ESI) m/z: 232(M+H)⁺.

### Step 5: 4-oxo-4,5,6,7-tetrahydrothieno[3,2-c]pyridine-2-carbonitrile (6)

To mixture of 2-bromo-6,7-dihydrothieno[3,2-c]pyridin-4(5H)-one (400 mg, 1.73 mmol) and Zn(CN)₂ (607 mg, 5.19 mmol) in DMF (5 mL) was added Pd(PPh₃)₄ (400 mg, 0.34 mmol). The mixture was degassed under N₂ atmosphere for three times and stirred at 120° C. for 12 hours. The mixture was diluted with water and extracted with ethyl acetate twice. The combined organic layers were washed with brine, dried over anhydrous Na₂SO₄, filtered and concentrated to dryness under reduced pressure. The residue was purified by flash column chromatography on silica gel (petroleum ether:ethyl acetate=1:1) to give 4-oxo-4,5,6,7-tetrahydrothieno[3,2-c]pyridine-2-carbonitrile (300 mg, yield 97%) as white solid. LC/MS (ESI) m/z: 179(M+H)⁺.

### Step 6: tert-butyl ((4-oxo-4,5,6,7-tetrahydrothieno[3,2-c]pyridin-2-yl)methyl)carbamate (7)

To a mixture of 4-oxo-4,5,6,7-tetrahydrothieno[3,2-c]pyridine-2-carbonitrile (300 mg, 1.68 mmol) and di-tert-butyl dicarbonate (800 mg, 3.66 mmol) in THE (10 mL) was added Raney Nickel (500 mg) and the mixture was degassed and stirred under a H₂ balloon at 25° C. for 12 hours. The mixture was filtered, and the filtrate was diluted with ethyl acetate, washed with brine, dried over anhydrous Na₂SO₄, filtered and concentrated to dryness under reduced pressure. The residue was purified by flash column chromatography on silica gel (petroleum ether:ethyl acetate=1:1) to give tert-butyl ((4-oxo-4,5,6,7-tetrahydrothieno[3,2-c]pyridin-2-yl)methyl)carbamate (170 mg, yield 35%) as white solid. LC/MS (ESI) m/z: 227 (M+H−56)⁺.

### Step 7: tert-butyl ((4-thioxo-4,5,6,7-tetrahydrothieno[3,2-c]pyridin-2-yl)methyl)carbamate (8)

To a solution of tert-butyl ((4-oxo-4,5,6,7-tetrahydrothieno[3,2-c]pyridin-2-yl)methyl) carbamate (170 mg, 0.60 mmol) in pyridine (5 mL) was added P₄S₁₀ (230 mg, 0.60 mmol) and the mixture was stirred at 75° C. for 2 hours. The mixture was diluted with water and extracted with ethyl acetate twice. The combined organic layers were washed with brine, dried over anhydrous Na₂SO₄, filtered and concentrated to dryness under reduced pressure. The residue was purified by flash column chromatography on silica gel (petroleum ether:ethyl acetate=1:2) to give tert-butyl ((4-thioxo-4,5,6,7-tetrahydrothieno[3,2-c]pyridin-2-yl)methyl) carbamate (80 mg, yield 44.6%) as yellow solid. LC/MS (ESI) m/z: 143 (M+H−56)⁺.

### Step 8: tert-butyl ((4-(methylthio)-6,7-dihydrothieno[3,2-c]pyridin-2-yl)methyl)carbamate (9)

To a mixture of tert-butyl ((4-thioxo-4,5,6,7-tetrahydrothieno[3,2-c]pyridin-2-yl)methyl) carbamate (80 mg, 0.26 mmol) and K₂CO₃ (111 mg, 0.80 mmol) in acetone (3 mL) was added Mel (76 mg, 0.53 mmol) and the mixture was stirred at 25° C. for 2 hours. The resulting mixture was filtered, and filtered cake was washed with ethyl acetate (2×5 mL). The filtrate was concentrated under reduced pressure to give tert-butyl ((4-(methylthio)-6,7-dihydrothieno[3,2-c]pyridin-2-yl)methyl)carbamate (80 mg, yield 96.3%) as yellow oil. LC/MS (ESI) m/z: 313 (M+H)⁺.

### Step 9: tert-butyl ((4-amino-6,7-dihydrothieno[3,2-c]pyridin-2-yl)methyl)carbamate (10)

To a solution of tert-butyl ((4-(methylthio)-6,7-dihydrothieno[3,2-c]pyridin-2-yl)methyl)carbamate (80 mg, 0.25 mmol) in MeOH (2 mL) was added NH₃/MeOH (2 mL, 7M) and the mixture was stirred in a sealed tube at 75° C. for 4 hours. The mixture was concentrated to dryness under reduced pressure to give tert-butyl ((4-amino-6,7-dihydrothieno[3,2-c]pyridin-2-yl)methyl)carbamate (60 mg, yield 83.3%) as white solid, which was used directly in the next step. LC/MS (ESI) (m/z): 226 (M+H−56)⁺.

### Step 10: 2-(aminomethyl)-6,7-dihydrothieno[3,2-c]pyridin-4-amine hydrochloride (11)

To a solution of tert-butyl ((4-amino-6,7-dihydrothieno[3,2-c]pyridin-2-yl)methyl)carbamate (40 mg, 0.17 mmol) in DCM (2 mL) was added HCl/1,4-dioxane (1 mL, 4M) at 0° C. and the mixture was stirred at room temperature for 2 hours. The mixture was concentrated under reduced pressure to give 2-(aminomethyl)-6,7-dihydrothieno[3,2-c]pyridin-4-amine hydrochloride (40 mg, yield 100%) as white solid, which was directly used in the next reaction without purification. LC/MS (ESI) m/z: 182 (M+H)⁺.

### Step 11: (1S,3S,5S)-N-((4-amino-6,7-dihydrothieno[3,2-c]pyridin-2-yl)methyl)-5-methyl-2-((4-phenoxybenzoyl)glycyl)-2-azabicyclo[3.1.0]hexane-3-carboxamide (Compound 182)

To a mixture of 2-(aminomethyl)-6,7-dihydrothieno[3,2-c]pyridin-4-amine hydrochloride (24 mg, 0.11 mmol), (1 S,3S,5S)-5-methyl-2-((4-phenoxybenzoyl)glycyl)-2-azabicyclo[3.1.0]hexane-3-carboxylic acid (50 mg, 0.12 mmol) and HATU (60 mg, 0.15 mmol) in DMF (2 mL) was added DIPEA (40 mg, 0.31 mmol), and the mixture was stirred at 25° C. for 1 hour. The resulting mixture was diluted with water (10 mL) and extracted with ethyl acetate (3×10 mL). The combined organic layers were washed with brine, dried over anhydrous Na₂SO₄, filtered and concentrated to dryness under reduced pressure. The residue was purified by prep-TLC (DCM:MeOH=10:1) and further purified by prep-HPLC to give Compound 182 (2.2 mg, yield 3.6%) as a white solid. ¹H NMR (400 MHz, CD₃OD) δ 7.83 (d, J=8.9 Hz, 2H), 7.44-7.40 (m, 2H), 7.31 (s, 1H), 7.21 (t, J=7.5 Hz, 1H), 7.06 (d, J=7.6 Hz, 2H), 6.99 (d, J=8.9 Hz, 2H), 4.82 (s, 1H), 4.52 (s, 2H), 4.32 (s, 2H), 3.62 (t, J=7.2 Hz, 2H), 3.42 (d, J=3.5 Hz, 1H), 3.11 (dt, J=14.5, 4.4 Hz, 3H), 2.41 (t, J=13.1 Hz, 1H), 2.24-2.13 (m, 1H), 1.30 (s, 3H), 1.12 (d, J=3.4 Hz, 1H), 0.80-0.78 (m, 1H). LC/MS (ESI) m/z: 558 (M+H)⁺.

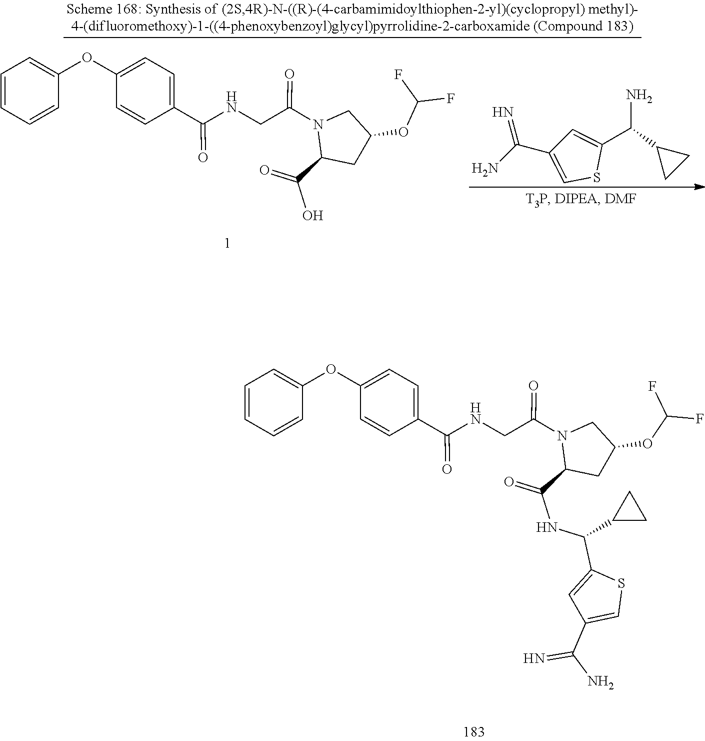 [SMILES: O=C(NCC(=O)N1C[C@H](OC(F)F)C[C@H]1C(=O)O)c1ccc(Oc2ccccc2)cc1 ; N=C(N)c1csc([C@H](N)C2CC2)c1 ; N=C(N)c1csc([C@H](NC(=O)[C@@H]2C[C@@H](OC(F)F)CN2C(=O)CNC(=O)c2ccc(Oc3ccccc3)cc2)C2CC2)c1]

### Step 1: (2S,4R)-N-((R)-(4-carbamimidoylthiophen-2-yl)(cyclopropyl)methyl)-4-(difluoromethoxy)-1-((4-phenoxybenzoyl)glycyl)pyrrolidine-2-carboxamide (Compound 183)

To a mixture of (2S,4R)-4-(difluoromethoxy)-1-((4-phenoxybenzoyl)glycyl)pyrrolidine-2-carboxylic acid (60 mg, 0.14 mmol) and (R)-5-(amino(cyclopropyl)methyl)thiophene-3-carboximidamide (50 mg, 0.14 mmol) in DMF (3.0 mL) was added DIPEA (0.25 mL, 0.83 mmol) and T₃P (132 mg, 0.21 mmol, 50% wt. in ethyl acetate) at 0° C. under N₂ atmosphere and the mixture was stirred at 25° C. for 2 hours. The mixture was concentrated to dryness under reduced pressure. The residue was purified by prep-TLC (DCM:MeOH=10:1) and further purified by prep-HPLC to give Compound 183 (6 mg, yield 7.1%) as a white solid. ¹H NMR (400 MHz, CD₃OD) δ 8.54 (s, 1H), 8.30-8.23 (m, 1H), 7.88-7.83 (m, 2H), 7.60 (dd, J=11.1, 9.8 Hz, 1H), 7.44-7.39 (m, 2H), 7.21 (dd, J=10.6, 4.2 Hz, 1H), 7.08-7.04 (m, 2H), 7.03-6.99 (m, 2H), 6.74-6.29 (m, 1H), 5.03 (s, 1H), 4.74-4.59 (m, 1H), 4.56-4.43 (m, 1H), 4.25 (t, J=22.1 Hz, 1H), 4.15 (d, J=16.7 Hz, 1H), 3.96 (dd, J=11.5, 4.6 Hz, 1H), 3.89 (d, J=11.1 Hz, 1H), 2.55-2.47 (m, 1H), 2.40-2.20 (m, 1H), 1.34-1.27 (m, 1H), 0.83-0.74 (m, 1H), 0.73-0.60 (m, 1H), 0.50 (ddt, J=23.5, 9.6, 4.7 Hz, 2H). LC/MS (ESI) m/z: 612 (M+H)⁺.

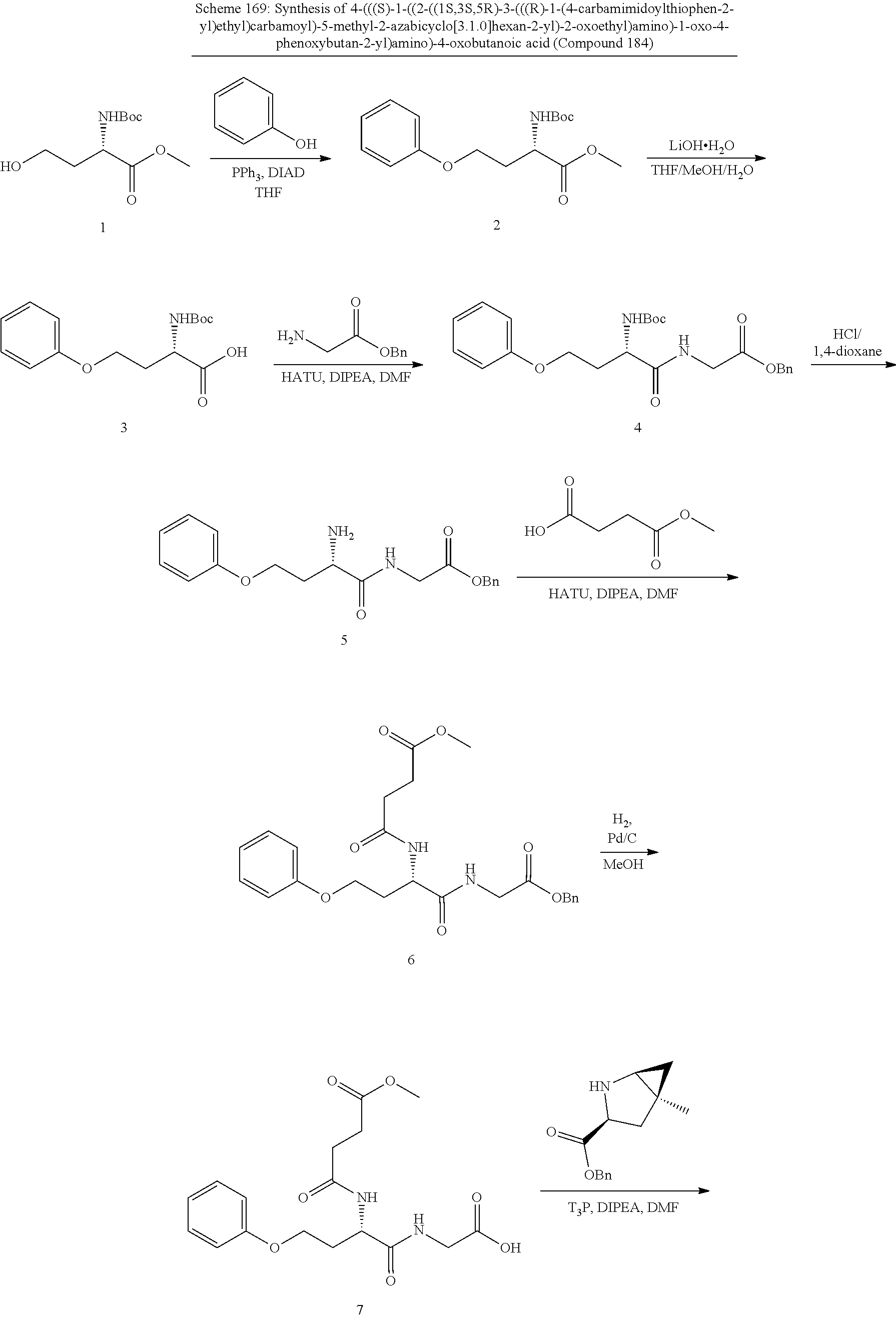

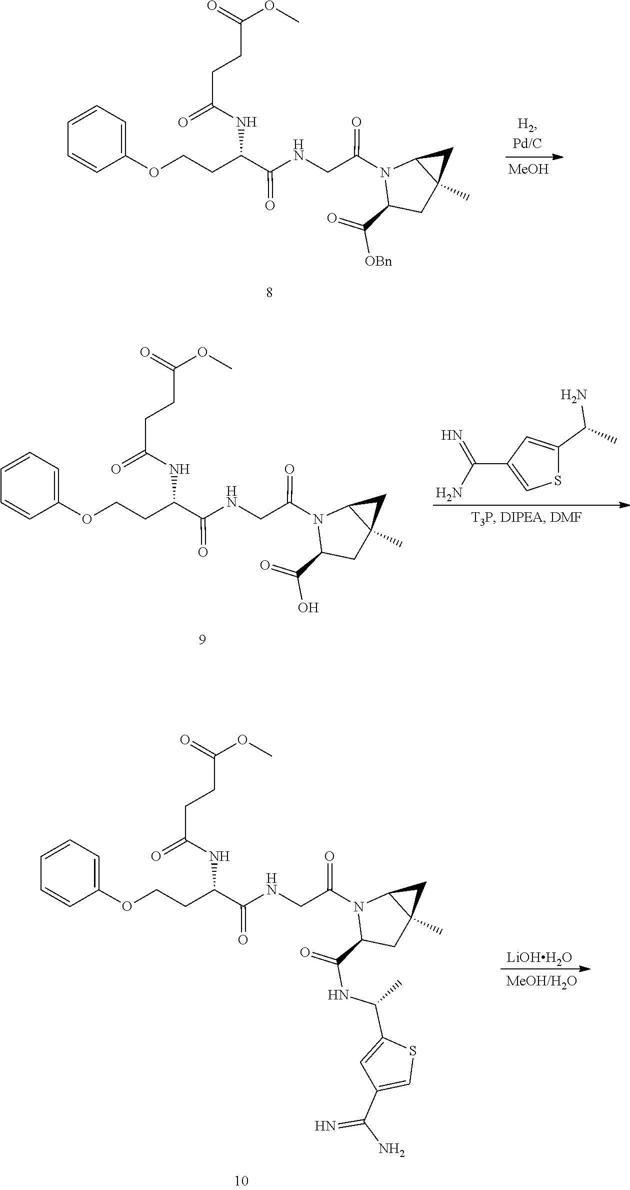 [SMILES: "MeOH" ; COC(=O)CCC(=O)N[C@@H](CCOc1ccccc1)C(=O)NCC(=O)N1[C@H]2C[C@@]2(C)C[C@H]1C(=O)OCc1ccccc1 ; COC(=O)CCC(=O)N[C@@H](CCOc1ccccc1)C(=O)NCC(=O)N1[C@H]2C[C@@]2(C)C[C@H]1C(=O)O ; C[C@@H](N)c1cc(C(=N)N)cs1 ; COC(=O)CCC(=O)N[C@@H](CCOc1ccccc1)C(=O)NCC(=O)N1[C@H]2C[C@@]2(C)C[C@H]1C(=O)N[C@H](C)c1cc(C(=N)N)cs1 ; "LiOH·H2O"]

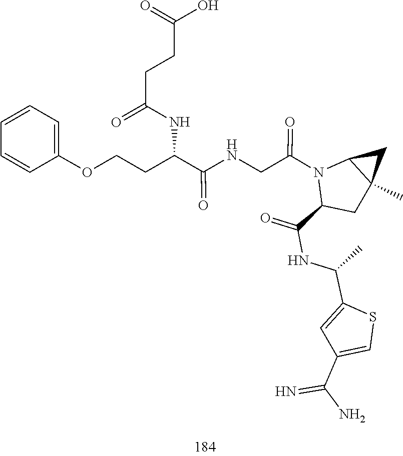 [SMILES: C[C@@H](NC(=O)[C@@H]1C[C@]2(C)C[C@@H]2N1C(=O)CNC(=O)[C@H](CCOc1ccccc1)NC(=O)CCC(=O)O)c1cc(C(=N)N)cs1]

### Step 1: methyl N-(tert-butoxycarbonyl)-O-phenyl-L-homoserinate (2)

To a mixture of methyl (tert-butoxycarbonyl)-L-homoserinate (400 mg, 1.7 mmol) and phenol (323 mg, 3.4 mmol) in THE (10.0 mL) was added PPh₃ (1.35 g, 5.1 mmol) and DIAD (1.04 g, 5.1 mmol) at 0° C. under N₂ atmosphere and the mixture was stirred at 30° C. for 16 hours. The mixture was diluted with water and extracted with ethyl acetate twice. The combined organic layers were dried over anhydrous Na₂SO₄, filtered and concentrated to dryness under reduced pressure. The residue was purified by column chromatography on silica gel (petroleum ether:ethyl acetate=10:1) to give methyl N-(tert-butoxycarbonyl)-O-phenyl-L-homoserinate (297 mg, yield 56.0%) as colorless oil. LC/MS (ESI) m/z: 310 (M+H)⁺.

### Step 2: N-(tert-butoxycarbonyl)-O-phenyl-L-homoserine (3)

To a solution of methyl N-(tert-butoxycarbonyl)-O-phenyl-L-homoserinate (297 mg, 0.96 mmol) in methanol (3 mL) and water (0.6 mL) was added lithium hydroxide (121 mg, 2.88 mmol) at 0° C. and the mixture was stirred at room temperature for 2 hours. The mixture was acidified with 1N aq.HCl to pH-3, extracted with ethyl acetate twice. The combined organic layers were washed with brine, dried over anhydrous Na₂SO₄, filtered and concentrated under reduced pressure to dryness to give N-(tert-butoxycarbonyl)-O-phenyl-L-homoserine (218 mg, yield 77.0%) as colorless oil, which was used directly in the next step. LC/MS (ESI) (m/z): 296 (M+H)⁺.

### Step 3: benzyl N-(tert-butoxycarbonyl)-O-phenyl-L-homoserylglycinate (4)

To a mixture of N-(tert-butoxycarbonyl)-O-phenyl-L-homoserine (218 mg, 0.74 mmol) and benzyl glycinate (225 mg, 1.11 mmol) in DMF (5.0 mL) was added DIPEA (0.6 mL, 0.55 mmol) and HATU (421 mg, 1.11 mmol) at 0° C. under N₂ atmosphere and the mixture was stirred at 25° C. for 2 hours.

The mixture was concentrated to dryness under reduced pressure. The residue was purified by flash chromatography (petroleum ether:ethyl acetate=4:1) to give benzyl N-(tert-butoxycarbonyl)-O-phenyl-L-homoserylglycinate (143 mg, yield 43.9%) as white solid. LC/MS (ESI) m/z: 443 (M+H)⁺.

### Step 4: benzyl O-phenyl-L-homoserylglycinate hydrochloride (5)

A solution of benzyl N-(tert-butoxycarbonyl)-O-phenyl-L-homoserylglycinate (143 mg, 0.32 mmol) in HCl/1,4-dioxane (3 mL) was stirred at room temperature for 2 hours. The reaction mixture was concentrated to dryness, co-evaporated with dichloromethane twice and dried under vacuum to give benzyl O-phenyl-L-homoserylglycinate hydrochloride (130 mg, yield 100%) as yellow solid, which was used directly in the next step. LC/MS (ESI) m/z: 343 (M+H)⁺.

### Step 5: methyl (S)-4-((1-((2-(benzyloxy)-2-oxoethyl)amino)-1-oxo-4-phenoxybutan-2-yl) amino)-4-oxobutanoate (6)

To a mixture of benzyl O-phenyl-L-homoserylglycinate hydrochloride (130 mg, 0.32 mmol) and 4-methoxy-4-oxobutanoic acid (64 mg, 0.48 mmol) in DMF (3.0 mL) was added DIPEA (0.27 mL, 1.60 mmol) and HATU (147 mg, 0.38 mmol) at 0° C. under N₂ atmosphere and the mixture was stirred at 25° C. for 2 hours. The mixture was diluted with EtOAc (20 mL), washed with water and brine, dried over Na₂SO₄, filtered and concentrated to dryness under reduced pressure. The residue was purified by flash chromatography (petroleum ether:ethyl acetate=4:1) to give methyl (S)-4-((1-((2-(benzyloxy)-2-oxoethyl)amino)-1-oxo-4-phenoxybutan-2-yl)amino)-4-oxobutanoate (140 mg, yield 95.9%) as colorless oil. LC/MS (ESI) m/z: 457 (M+H)⁺.

### Step 6: N-(4-methoxy-4-oxobutanoyl)-O-phenyl-L-homoserylglycine (7)

To a solution of methyl (S)-4-((1-((2-(benzyloxy)-2-oxoethyl)amino)-1-oxo-4-phenoxybutan-2-yl)amino)-4-oxobutanoate (167 mg, 0.37 mmol) in MeOH (5 mL) was added Pd/C (20 mg, 10% wt.) at 0° C., and the reaction mixture was stirred under H₂ atmosphere at 25° C. for 2 hours. The mixture was filtered and the filtrate was concentrated to dryness under reduced pressure give N-(4-methoxy-4-oxobutanoyl)-O-phenyl-L-homoserylglycine (127 mg, yield 94.8%) as light yellow oil, which was used directly in the next step. LC/MS (ESI) m/z: 367 (M+H)⁺.

### Step 7: benzyl (1S,3S,5S)-2-(N-(4-methoxy-4-oxobutanoyl)-O-phenyl-L-homoserylglycyl)-5-methyl-2-azabicyclo[3.1.0]hexane-3-carboxylate (8)

To a mixture of N-(4-methoxy-4-oxobutanoyl)-O-phenyl-L-homoserylglycine (127 mg, 0.35 mmol) and benzyl (1S,3S,5S)-5-methyl-2-azabicyclo[3.1.0]hexane-3-carboxylate (120 mg, 0.52 mmol) in DMF (3.0 mL) was added DIPEA (0.34 mL, 2.10 mmol) and T₃P (331 mg, 0.52 mmol, 50% wt. in ethyl acetate) at 0° C. under N₂ atmosphere and the mixture was stirred at 25° C. for 2 hours. The mixture was concentrated to dryness under reduced pressure. The residue was purified by flash chromatography (petroleum ether:ethyl acetate=1:1) to give benzyl (1S,3S,5S)-2-(N-(4-methoxy-4-oxobutanoyl)-O-phenyl-L-homoserylglycyl)-5-methyl-2-azabicyclo[3.1.0]hexane-3-carboxylate (150 mg, yield 75.0%) as colorless oil. LC/MS (ESI) m/z: 580 (M+H)⁺.

### Step 8: (1S,3S,5S)-2-(N-(4-methoxy-4-oxobutanoyl)-O-phenyl-L-homoserylglycyl)-5-methyl-2-azabicyclo[3.1.0]hexane-3-carboxylic acid (9)

To a solution of benzyl (1 S,3S,5S)-2-(N-(4-methoxy-4-oxobutanoyl)-O-phenyl-L-homoserylglycyl)-5-methyl-2-azabicyclo[3.1.0]hexane-3-carboxylate (150 mg, 0.26 mmol) in MeOH (5 mL) was added Pd/C (10 mg, 10% wt.) at 0° C., and the reaction mixture was stirred under H₂ atmosphere at 25° C. for 2 hours. The mixture was filtered and the filtrate was concentrated to dryness under reduced pressure give (1 S,3S,5S)-2-(N-(4-methoxy-4-oxobutanoyl)-O-phenyl-L-homoserylglycyl)-5-methyl-2-azabicyclo[3.1.0]hexane-3-carboxylic acid (120 mg, yield 95.2%) as light oil, which was used directly in the next step. LC/MS (ESI) m/z: 490 (M+H)⁺.

### Step 9: benzyl methyl 4-(((S)-1-((2-((1S,3S,5S)-3-(((R)-1-(4-carbamimidoylthiophen-2-yl) ethyl)carbamoyl)-5-methyl-2-azabicyclo[3.1.0]hexan-2-yl)-2-oxoethyl)amino)-1-oxo-4-phenoxybutan-2-yl)amino)-4-oxobutanoate (10)

To a mixture of (1 S,3S,5S)-2-(N-(4-methoxy-4-oxobutanoyl)-O-phenyl-L-homoserylglycyl)-5-methyl-2-azabicyclo[3.1.0]hexane-3-carboxylic acid (65 mg, 0.13 mmol) and (R)-5-(1-aminoethyl)thiophene-3-carboximidamide (34 mg, 0.20 mmol) in DMF (3.0 mL) was added DIPEA (0.11 mL, 0.65 mmol) and T₃P (101 mg, 0.16 mmol, 50% wt. in ethyl acetate) at 0° C. under N₂ atmosphere and the mixture was stirred at 25° C. for 2 hours. The mixture was concentrated to dryness under reduced pressure. The residue was purified by pre-TLC (DCM:MeOH=10:1) to give methyl 4-(((S)-1-((2-((1S,3S,5S)-3-(((R)-1-(4-carbamimidoylthiophen-2-yl)ethyl)carbamoyl)-5-methyl-2-azabicyclo[3.1.0]hexan-2-yl)-2-oxoethyl)amino)-1-oxo-4-phenoxybutan-2-yl)amino)-4-oxobutanoate (70 mg, yield 82.3%) as colorless oil. LC/MS (ESI) m/z: 641 (M+H)⁺.

### Step 10: 4-(((S)-1-((2-((1S,3S,5S)-3-(((R)-1-(4-carbamimidoylthiophen-2-yl)ethyl)carbamoyl)-5-methyl-2-azabicyclo[3.1.0]hexan-2-yl)-2-oxoethyl)amino)-1-oxo-4-phenoxybutan-2-yl)amino)-4-oxobutanoic acid (Compound 184)

To a solution of methyl 4-(((S)-1-((2-((1S,3S,5S)-3-(((R)-1-(4-carbamimidoylthiophen-2-yl) ethyl)carbamoyl)-5-methyl-2-azabicyclo[3.1.0]hexan-2-yl)-2-oxoethyl)amino)-1-oxo-4-phenoxybutan-2-yl)amino)-4-oxobutanoate (70 mg, 0.11 mmol) in MeOH (3 mL) was added a solution of LiOH·H₂O (6 mg, 0.16 mmol) in water (0.6 mL) and the mixture was stirred at 30° C. for 2 hours. The mixture was acidified with 1 N aq.HCl to pH-4 and purified by prep-HPLC to give Compound 184 (6 mg, yield 8.8%) as a white solid. ¹H NMR (400 MHz, CD₃OD) δ 8.43 (s, 1H), 8.20 (d, J=1.6 Hz, 1H), 7.64 (s, 1H), 7.26-7.21 (m, 2H), 6.91-6.88 (m, 3H), 5.11 (q, J=7.2 Hz, 1H), 4.81 (d, J=3.6 Hz, 1H), 4.72 (dd, J=9.6, 4.4 Hz, 1H), 4.31 (d, J=16.6 Hz, 1H), 4.06 (ddd, J=11.1, 8.9, 4.7 Hz, 2H), 3.98 (d, J=16.6 Hz, 1H), 3.35 (dd, J=5.9, 2.3 Hz, 1H), 2.62-2.55 (m, 2H), 2.52-2.40 (m, 4H), 2.12 (dd, J=13.3, 3.7 Hz, 1H), 2.04 (ddd, J=14.5, 9.7, 5.0 Hz, 1H), 1.56 (t, J=9.2 Hz, 3H), 1.29 (s, 4H), 0.80 (t, J=5.4 Hz, 1H). LC/MS (ESI) (m/z): 627 (M+H)⁺.

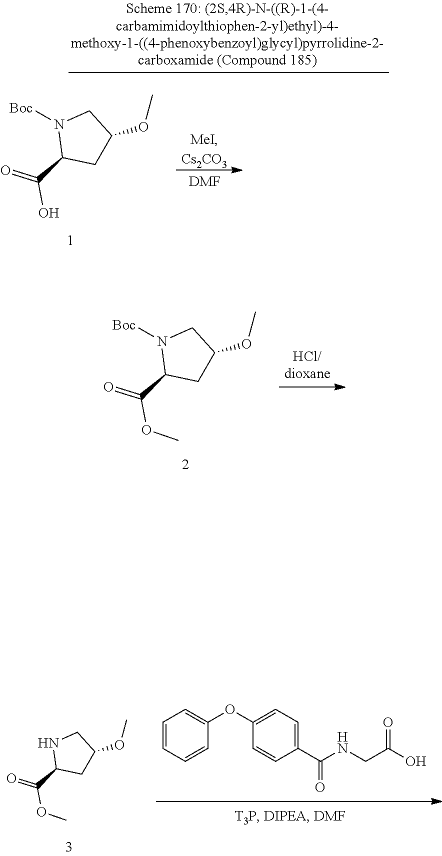

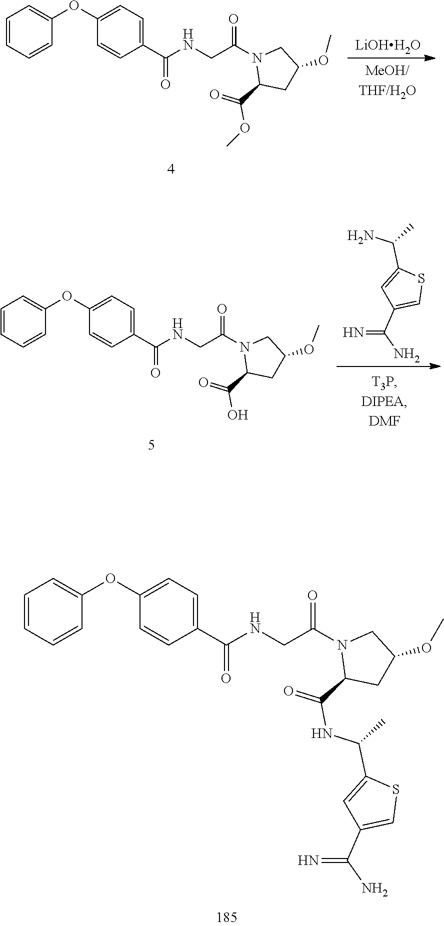 [SMILES: COC(=O)[C@@H]1C[C@@H](OC)CN1C(=O)CNC(=O)c1ccc(Oc2ccccc2)cc1 ; "LiOH·H2O" ; "MeOH/THF/H2O" ; CO[C@@H]1C[C@@H](C(=O)O)N(C(=O)CNC(=O)c2ccc(Oc3ccccc3)cc2)C1 ; C[C@@H](N)c1cc(C(=N)N)cs1 ; CO[C@@H]1C[C@@H](C(=O)N[C@H](C)c2cc(C(=N)N)cs2)N(C(=O)CNC(=O)c2ccc(Oc3ccccc3)cc2)C1]

### Step 1: 1-(tert-butyl) 2-methyl (2S,4R)-4-methoxypyrrolidine-1,2-dicarboxylate (2)

At 0° C., to a solution of (2S,4R)-1-(tert-butoxycarbonyl)-4-methoxypyrrolidine-2-carboxylic acid (100 mg, 0.41 mmol) in DMF (2 mL) was added Cs₂CO₃ (147 mg, 0.45 mmol) followed by Mel (0.03 mL, 0.49 mmol), and the mixture was stirred at 25° C. for 16 hours. The mixture was diluted with ethyl acetate twice and washed with brine, dried over anhydrous Na₂SO₄, filtered and concentrated to dryness to give 1-(tert-butyl) 2-methyl (2S,4R)-4-methoxypyrrolidine-1,2-dicarboxylate (117 mg, yield 100%) as yellow oil. LC/MS (ESI) m/z: 260 (M+H)⁺.

### Step 2: methyl (2S,4R)-4-methoxypyrrolidine-2-carboxylate (3)

At 0° C., a solution of 1-(tert-butyl) 2-methyl (2S,4R)-4-methoxypyrrolidine-1,2-dicarboxylate (117 mg, 0.45 mmol) in HCl/1,4-dioxane (3 mL) was stirred at 25° C. for 3 hours. The reaction mixture was concentrated to dryness, washed with DCM and dried under vacuum to give methyl (2S,4R)-4-methoxypyrrolidine-2-carboxylate (71 mg, yield 98.6%) as light oil. LC/MS (ESI) m/z: 160 (M+H)⁺.

### Step 3: methyl (2S,4R)-4-methoxy-1-((4-phenoxybenzoyl)glycyl)pyrrolidine-2-carboxylate (4)

At 0° C., to a mixture of (2S,4R)-1-(tert-butoxycarbonyl)-4-methoxypyrrolidine-2-carboxylic acid (71 mg, 0.45 mmol) and (4-phenoxybenzoyl)glycine (121 mg, 0.45 mmol) in DMF (2 mL) was added T₃P (859 mg, 1.35 mmol, 50% wt. in ethyl acetate) and DIPEA (0.4 mL, 2.70 mmol) under N₂ atmosphere and the mixture was stirred at room temperature overnight. The mixture was diluted with ethyl acetate and washed with saturated aq.NaHCO₃ solution. The aqueous layer was extracted with ethyl acetate and the combined organic layers were washed with brine, dried over anhydrous Na₂SO₄, filtered and concentrated to dryness under reduced pressure. The residue was purified by flash chromatography on silica gel (dichloromethane:methanol=20:1) to afford ethyl methyl (2S,4R)-4-methoxy-1-((4-phenoxybenzoyl)glycyl)pyrrolidine-2-carboxylate (130 mg, yield 70.3%) as yellow oil. LC/MS (ESI) m/z: 413 (M+H)⁺.

### Step 4: (2S,4R)-4-methoxy-1-((4-phenoxybenzoyl)glycyl)pyrrolidine-2-carboxylic acid (5)

To a solution of ethyl methyl (2S,4R)-4-methoxy-1-((4-phenoxybenzoyl)glycyl)pyrrolidine-2-carboxylate (130 mg, 0.32 mmol) in THF/MeOH/H₂O (4 mL, v:v=2/1/1) was added lithium hydroxide hydrate (13 mg, 0.32 mmol) and the reaction mixture was stirred at room temperature for 3 hours. The mixture was concentrated to dryness, diluted with water and washed with ethyl acetate twice. The aqueous layer was acidified with 1N aq.HCl to pH-4 and extracted with DCM twice. The combined organic layers were washed with brine, dried over anhydrous Na₂SO₄, filtered and concentrated to dryness to afford (2S,4R)-4-methoxy-1-((4-phenoxybenzoyl)glycyl)pyrrolidine-2-carboxylic acid (120 mg, yield 94.5%) as white solid. LC/MS (ESI) m/z: 399 (M+H)⁺.

### Step 5: (2S,4R)-N-((R)-1-(4-carbamimidoylthiophen-2-yl)ethyl)-4-methoxy-1-((4-phenoxybenzoyl)glycyl)pyrrolidine-2-carboxamide (Compound 185)

At 0° C., to a solution of (2S,4R)-4-methoxy-1-((4-phenoxybenzoyl)glycyl)pyrrolidine-2-carboxylic acid (50 mg, 0.13 mmol) and (R)-5-(1-aminoethyl)thiophene-3-carboximidamide (25 mg, 0.15 mmol) in DMF (2 mL) was added DIPEA (0.06 mL, 0.39 mmol) and T₃P (124 mg, 0.20 mmol, 50% wt. in ethyl acetate) under N₂ atmosphere and the reaction mixture was stirred at room temperature for 2 hours. The mixture was diluted with ethyl acetate and washed with saturated aq.NaHCO₃ solution. The aqueous layer was extracted with ethyl acetate twice and the combined organic layers were washed with brine, dried over anhydrous Na₂SO₄, filtered and concentrated to dryness under reduced pressure. The residue was purified by prep-HPLC to give Compound 185 (1.8 mg, yield 2.5%) as a white solid. ¹H NMR (400 MHz, CD₃OD) δ 8.51 (s, 1H), 8.22 (dd, J=7.6, 7.6 Hz, 1H), 7.88-7.82 (m, 2H), 7.54 (dd, J=6.4, 2.82 Hz, 1H), 7.44-7.38 (m, 2H), 7.20 (t, J=7.4 Hz, 1H), 7.06 (dd, J=8.8, 8.4 Hz, 2H), 7.02-6.98 (m, 2H), 5.31-5.22 (m, 1H), 4.48 (t, J=8.0 Hz, 1H), 4.19 (t, J=8.4 Hz, 2H), 3.86-3.74 (m, 2H), 3.37 (s, 3H), 2.59-2.32 (m, 1H), 2.20-2.01 (m, 1H), 1.59 (t, J=15.0 Hz, 3H).LC/MS (ESI) m/z: 550 (M+H)⁺.

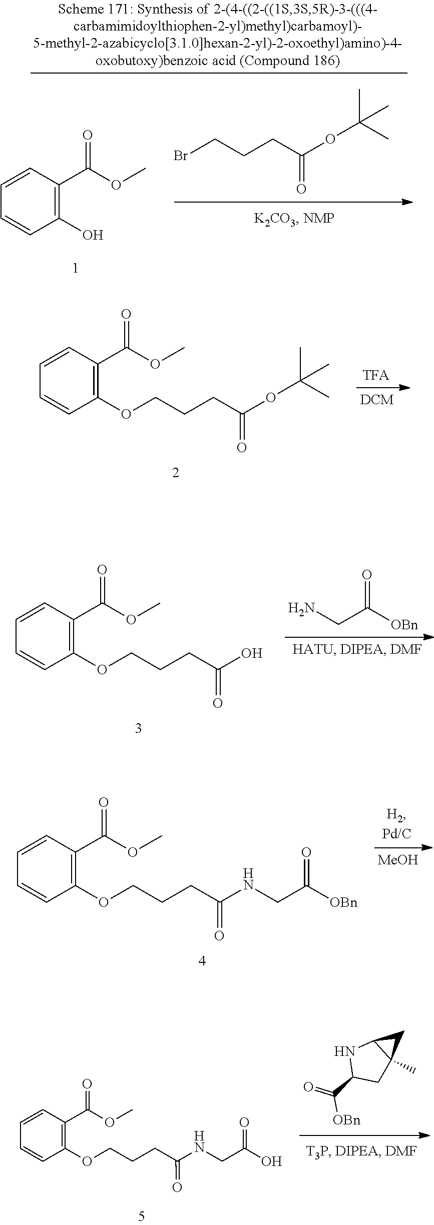

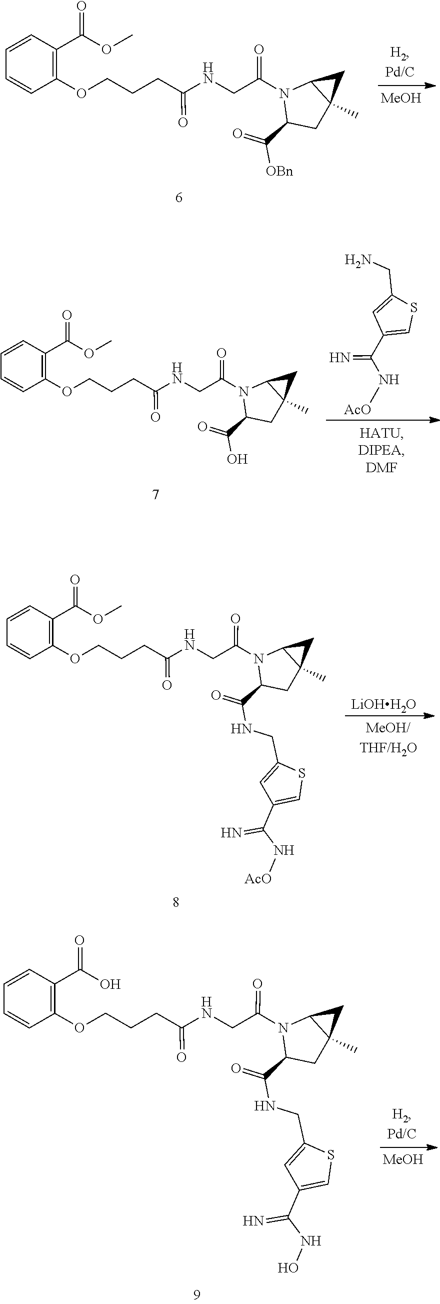

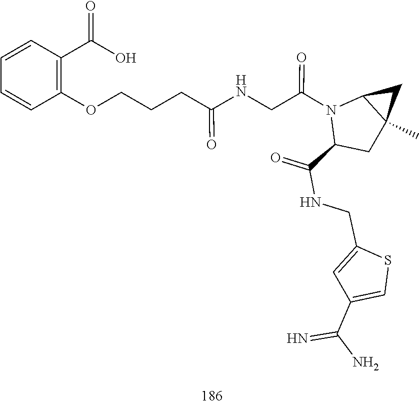 [SMILES: C[C@@]12C[C@@H]1N(C(=O)CNC(=O)CCCOc1ccccc1C(=O)O)[C@H](C(=O)NCc1cc(C(=N)N)cs1)C2]

### Step 1: methyl 2-(4-(tert-butoxy)-4-oxobutoxy)benzoate (2)

To a mixture of methyl 2-hydroxybenzoate (1.0 g, 6.58 mmol) and tert-butyl 4-bromobutanoate (1.5 g, 6.58 mmol) in NMP (10 mL) was added K₂CO₃ (2.7 g, 19.7 mmol) at 0° C. and the mixture was stirred at 120° C. for 24 hours. The mixture was diluted with ethyl acetate, washed with water and brine, dried over anhydrous Na₂SO₄, filtered and concentrated to dryness. The residue was purified by flash chromatography on silica gel (petroleum ether:ethyl acetate=5:1) to give methyl 2-(4-(tert-butoxy)-4-oxobutoxy) benzoate (1.0 g, yield 53.6%) as light oil. LC/MS (ESI) m/z: 239 (M−56+H)⁺.

### Step 2: 4-(2-(methoxycarbonyl)phenoxy)butanoic acid (3)

To a solution of methyl 2-(4-(tert-butoxy)-4-oxobutoxy)benzoate (1.0 g, 3.54 mmol) in DCM (10 mL) was added TFA (3 mL) at 0° C. and the mixture was stirred at 25° C. for 3 hours. The reaction mixture was concentrated to dryness, co-evaporated with dichloromethane twice and dried under vacuum to give 4-(2-(methoxycarbonyl)phenoxy)butanoic acid (842 mg, yield 99.9%) as yellow oil, which was used in next step without purification. LC/MS (ESI) m/z: 239 (M+H)⁺.

### Step 3: methyl 2-(4-((2-(benzyloxy)-2-oxoethyl)amino)-4-oxobutoxy)benzoate (4)

To a mixture of 4-(2-(methoxycarbonyl)phenoxy)butanoic acid (842 mg, 3.54 mmol) in DMF (9 mL) was added DIPEA (2.3 mL, 14.2 mmol) followed by HATU (2.0 g, 5.31 mmol) at 0° C., and the reaction mixture was stirred at 25° C. for 3 hours. The mixture was diluted with ethyl acetate, washed with water and brine, dried over anhydrous Na₂SO₄, filtered and concentrated to dryness. The residue was purified by flash chromatography on silica gel (dichloromethane:methanol=20:1) to give methyl 2-(4-((2-(benzyloxy)-2-oxoethyl) amino)-4-oxobutoxy)benzoate (850 mg, yield 65.4%) as light oil.

LC/MS (ESI) m/z: 384 (M+H)⁺.

### Step 4: (4-(2-(methoxycarbonyl)phenoxy)butanoyl)glycine (5)

To a solution of methyl 2-(4-((2-(benzyloxy)-2-oxoethyl)amino)-4-oxobutoxy)benzoate (850 mg, 2.21 mmol) in MeOH (8 mL) were added Pd/C (50 mg, 10% wt.) at room temperature under N₂ atmosphere and the reaction mixture was stirred at room temperature under H₂ atmosphere for 3 hours. The mixture was filtered and the filtrate was concentrated to dryness under reduced pressure to give (4-(2-(methoxycarbonyl)phenoxy)butanoyl)glycine (595 mg, yield 91.4%) as light oil, which was used directly in the next step. LC/MS (ESI) m/z: 296 (M+H)⁺.

### Step 5: benzyl (1S,3S,5S)-2-((4-(2-(methoxycarbonyl)phenoxy)butanoyl)glycyl)-5-methyl-2-azabicyclo[3.1.0]hexane-3-carboxylate (6)

To a mixture of (4-(2-(methoxycarbonyl)phenoxy)butanoyl)glycine (260 mg, 0.90 mmol) and benzyl (1S,3S,5S)-5-methyl-2-azabicyclo[3.1.0]hexane-3-carboxylate (209 mg, 0.90 mmol) in DMF (3 mL) was added DIPEA (0.9 mL, 5.40 mmol) followed by T₃P (1.7 g, 2.70 mmol, 50% wt. in ethyl acetate) at 0° C., and the mixture was stirred at 30° C. for 16 hours. The mixture was washed with saturated aq.NaHCO₃ solution and the aqueous layer was extracted with ethyl acetate twice. The combined organic layers were washed with brine, dried over anhydrous Na₂SO₄, filtered and concentrated to dryness. The residue was purified by flash chromatography on silica gel (dichloromethane:methanol=20:1) to give benzyl (1S,3S,5S)-2-((4-(2-(methoxycarbonyl)phenoxy)butanoyl)glycyl)-5-methyl-2-azabicyclo[3.1.0]hexane-3-carboxylate (432 mg, yield 94.5%) as light oil. LC/MS (ESI) m/z: 509 (M+H)⁺.

### Step 6: (1S,3S,5S)-2-((4-(2-(methoxycarbonyl)phenoxy)butanoyl)glycyl)-5-methyl-2-azabicyclo[3.1.0]hexane-3-carboxylic acid (7)

To a solution of benzyl (1 S,3S,5S)-2-((4-(2-(methoxycarbonyl)phenoxy)butanoyl)glycyl)-5-methyl-2-azabicyclo[3.1.0]hexane-3-carboxylate (432 mg, 0.85 mmol) in methanol (5 mL) were added Pd/C (40 mg, 10% wt.) under N₂ atmosphere and the reaction mixture was stirred at room temperature under H₂ atmosphere for 1.5 hours. The mixture was filtered and the filtrate was concentrated to dryness under reduced pressure to give (1 S,3S,5S)-2-((4-(2-(methoxycarbonyl)phenoxy)butanoyl)glycyl)-5-methyl-2-azabicyclo[3.1.0]hexane-3-carboxylic acid (328 mg, yield 92.4%) as light yellow oil. LC/MS (ESI) m/z: 419 (M+H)⁺.

### Step 7: methyl 2-(4-((2-((1S,3S,5S)-3-(((4-(N-acetoxycarbamimidoyl)thiophen-2-yl)methyl) carbamoyl)-5-methyl-2-azabicyclo[3.1.0]hexan-2-yl)-2-oxoethyl)amino)-4-oxobutoxy)benzoate (8)

To a mixture of (1S,3S,5S)-2-((4-(2-(methoxycarbonyl)phenoxy)butanoyl)glycyl)-5-methyl-2-azabicyclo[3.1.0]hexane-3-carboxylic acid (100 mg, 0.24 mmol) and N-acetoxy-5-(aminomethyl)thiophene-3-carboximidamide (101 mg, 0.48 mmol) in DMF (2 mL) was added DIPEA (0.2 mL, 1.20 mmol) followed by HATU (134 mg, 0.36 mmol) at 0° C., and the mixture was stirred at 25° C. for 3 hours. The mixture was extracted ethyl acetate twice and washed with brine, dried over anhydrous Na₂SO₄, filtered and concentrated to dryness. The residue was purified by flash chromatography on silica gel (dichloromethane:methanol=20:1) to give methyl 2-(4-((2-((1 S,3S,5S)-3-(((4-(N-acetoxycarbamimidoyl)thiophen-2-yl) methyl)carbamoyl)-5-methyl-2-azabicyclo[3.1.0]hexan-2-yl)-2-oxoethyl)amino)-4-oxobutoxy)benzoate (120 mg, yield 81.6%) as yellow oil. LC/MS (ESI) m/z: 614 (M+H)⁺.

### Step 8: 2-(4-((2-((1S,3S,5S)-3-(((4-(N-hydroxycarbamimidoyl)thiophen-2-yl)methyl) carbamoyl)-5-methyl-2-azabicyclo[3.1.0]hexan-2-yl)-2-oxoethyl)amino)-4-oxobutoxy)benzoic acid (9)

To a solution of methyl 2-(4-((2-((1 S,3S,5S)-3-(((4-(N-acetoxycarbamimidoyl)thiophen-2-yl) methyl)carbamoyl)-5-methyl-2-azabicyclo[3.1.0]hexan-2-yl)-2-oxoethyl)amino)-4-oxobutoxy)benzoate (120 mg, 0.20 mmol) in MeOH (1 mL) and THE (0.5 mL) was added a solution of LiOH·H₂O (12 mg, 0.30 mmol) in water (0.5 mL) at 0° C. and the mixture was stirred at 30° C. for 24 hours. The mixture was diluted with water and washed with ethyl acetate twice. The aqueous layer was acidified with 1 M aq.HCl and extracted with ethyl acetate twice. The combined organic layers were washed with brine, dried with anhydrous Na₂SO₄, filtered and concentrated to dryness to give 2-(4-((2-((1S,3S,5S)-3-(((4-(N-hydroxycarbamimidoyl) thiophen-2-yl)methyl)carbamoyl)-5-methyl-2-azabicyclo[3.1.0]hexan-2-yl)-2-oxoethyl)amino)-4-oxobutoxy)benzoic acid (111 mg, yield 99.6%) as yellow oil, which was used directly in the next step. LC/MS (ESI) m/z: 558 (M+H)⁺.

### Step 9: 2-(4-((2-((1S,3S,5S)-3-(((4-carbamimidoylthiophen-2-yl)methyl)carbamoyl)-5-methyl-2-azabicyclo[3.1.0]hexan-2-yl)-2-oxoethyl)amino)-4-oxobutoxy)benzoic acid (Compound 186)

To a solution of 2-(4-((2-((1 S,3S,5S)-3-(((4-(N-hydroxycarbamimidoyl)thiophen-2-yl)methyl) carbamoyl)-5-methyl-2-azabicyclo[3.1.0]hexan-2-yl)-2-oxoethyl)amino)-4-oxobutoxy)benzoic acid (30 mg, 0.05 mmol) in MeOH (2 mL) was added Pd/C (5 mg, 10% wt.) under N₂ atmosphere and the reaction mixture was stirred at room temperature under H₂ atmosphere for 16 hours. The mixture was filtered and the filtrate was concentrated to dryness under reduced pressure. The residue was purified by prep-HPLC to give 2-(4-((2-((1 S,3S,5S)-3-(((4-carbamimidoylthiophen-2-yl)methyl)carbamoyl)-5-methyl-2-azabicyclo[3.1.0]hexan-2-yl)-2-oxoethyl)amino)-4-oxobutoxy)benzoic acid (1.7 mg, yield 6.3%) as white solid. ¹H NMR (400 MHz, CD₃OD) δ 8.35 (s, 1H), 8.20 (d, J=1.6 Hz, 1H), 7.62 (d, J=1.2 Hz, 1H), 7.47 (dd, J=6.8, 7.6 Hz, 1H), 7.31-7.27 (m, 1H), 6.99 (d, J=8.0 Hz, 1H), 6.93 (t, J=7.4 Hz, 1H), 4.80 (d, J=4.0 Hz, 1H), 4.66 (d, J=17.2 Hz, 1H), 4.31 (dd, J=29.2, 29.2 Hz, 2H), 4.12 (s, 1H), 4.08-4.05 (m, 2H), 3.34 (s, 1H), 2.59-2.54 (m, 2H), 2.46-2.39 (m, 1H), 2.15-2.10 (m, 1H), 2.08-2.04 (m, 2H), 1.29 (s, 3H), 1.27-1.24 (m, 1H), 0.83 (t, J=5.9 Hz, 1H). LC/MS (ESI) m/z: 542 (M+H)⁺.

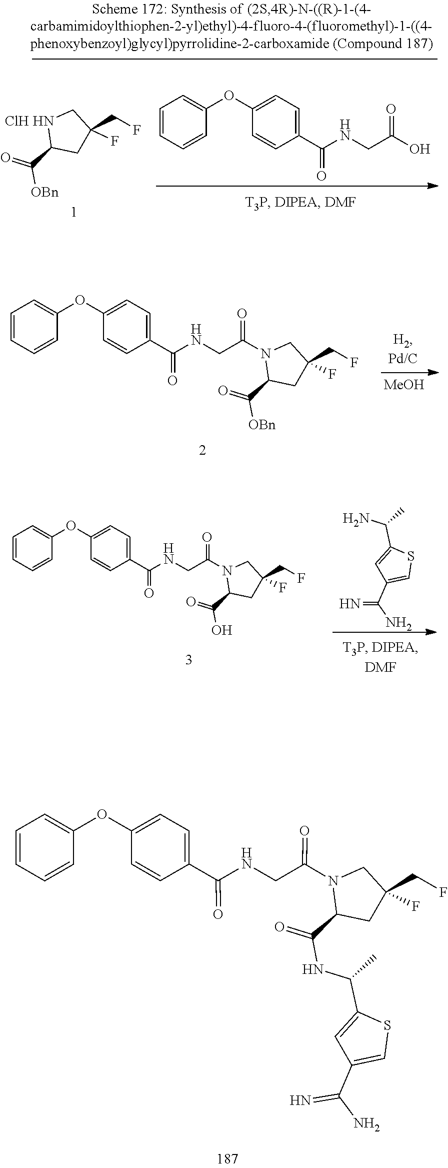

### Step 1: benzyl (2S,4R)-4-fluoro-4-(fluoromethyl)-1-((4-phenoxybenzoyl)glycyl)pyrrolidine-2-carboxylate

To a mixture of benzyl (2S,4R)-4-fluoro-4-(fluoromethyl)pyrrolidine-2-carboxylate hydrochloride (30 mg, 0.12 mmol) and (4-phenoxybenzoyl)glycine (32 mg, 0.12 mmol) in DMF (2 mL) was added DIPEA (0.12 mL, 0.72 mmol) and T₃P (224 mg, 0.354 mmol, 50% wt. in ethyl acetate) at 0° C. under N₂ atmosphere and the mixture was stirred at 25° C. for 2 hours. The mixture was diluted with ethyl acetate and washed with saturated aq.NaHCO₃ solution. The aqueous layer was extracted with ethyl acetate twice. The combined organic layers were washed with brine, dried over anhydrous Na₂SO₄, filtered and concentrated to dryness under reduced pressure. The residue was purified by flash chromatography on silica gel (petroleum ether:ethyl acetate=1:1) to give benzyl (2S,4R)-4-fluoro-4-(fluoromethyl)-1-((4-phenoxybenzoyl)glycyl)pyrrolidine-2-carboxylate (59 mg, yield 98.7%) as yellow oil. LC/MS (ESI) (m/z): 508 (M+H)⁺.

### Step 2: (2S,4R)-4-fluoro-4-(fluoromethyl)-1-((4-phenoxybenzoyl)glycyl)pyrrolidine-2-carboxylic acid

To a solution of benzyl (2S,4R)-4-fluoro-4-(fluoromethyl)-1-((4-phenoxybenzoyl)glycyl)pyrrolidine-2-carboxylate (59 mg, 0.012 mmol) in MeOH (3 mL) was added Pd/C (10% wt., 15 mg) at 0° C., and the mixture was degassed under N₂ atmosphere for three times and stirred under a H₂ balloon at 25° C. for 1 hour. The mixture was filtered and the filtrate was concentrated under reduce pressure to give ((2S,4R)-4-fluoro-4-(fluoromethyl)-1-((4-phenoxybenzoyl)glycyl)pyrrolidine-2-carboxylic acid (48 mg, yield 98.8%) as light oil, which was used directly in the next step without further purification. LC/MS (ESI) (m/z): 419 (M+H)⁺.

### Step 3: (2S,4R)-N-((R)-1-(4-carbamimidoylthiophen-2-yl)ethyl)-4-fluoro-4-(fluoromethyl)-1-((4-phenoxybenzoyl)glycyl)pyrrolidine-2-carboxamide (Compound 187)

To a mixture of ((2S,4R)-4-fluoro-4-(fluoromethyl)-1-((4-phenoxybenzoyl)glycyl)pyrrolidine-2-carboxylic acid (48 mg, 0.11 mmol) and (R)-5-(1-aminoethyl)thiophene-3-carboximidamide (23 mg, 0.14 mmol) in DMF (2 mL) was added DIPEA (0.09 mL, 0.55 mmol) and T₃P (105 mg, 0.17 mmol, 50% wt. in ethyl acetate) at 0° C. and the mixture was stirred at 25° C. for 1 hour. The mixture was washed with water and extracted with ethyl acetate twice. The combined organic layers were washed with brine, dried over anhydrous Na₂SO₄, filtered and concentrated to dryness under reduced pressure. The residue was purified by pre-HPLC to give Compound 187 (5.0 mg, yield 7.7%) as a white solid. ¹H NMR (400 MHz, CD₃OD) δ 8.23 (d, J=10.4 Hz, 1H), 7.84 (t, J=8.0 Hz, 2H), 7.56 (d, J=9.6 Hz, 1H), 7.44-7.39 (m, 2H), 7.21 (t, J=7.4 Hz, 1H), 7.06 (d, J=7.6 Hz, 2H), 7.01 (d, J=8.8 Hz, 2H), 5.39-5.22 (m, 1H), 4.80-4.74 (m, 1H), 4.69-4.59 (m, 2H), 4.36-4.22 (m, 1H), 4.18-4.08 (m, 2H), 4.01-3.86 (m, 1H), 2.83-2.53 (m, 1H), 2.44-2.13 (m, 1H), 1.62 (dd, J=29.6, 29.6 Hz, 3H). LC/MS (ESI) (m/z): 570 (M+H)⁺.

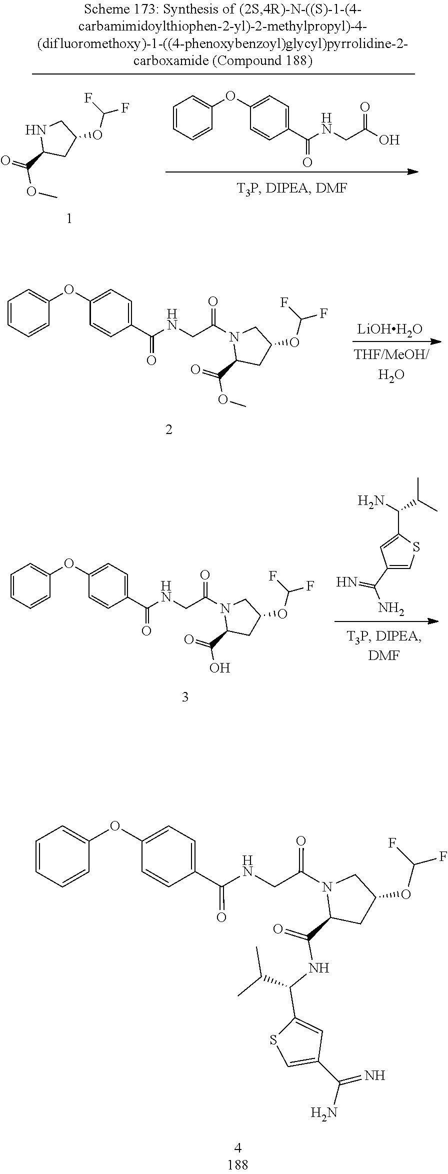

### Step 1: methyl (2S,4R)-4-(difluoromethoxy)-1-((4-phenoxybenzoyl)glycyl)pyrrolidine-2-carboxylate (2)

At 0° C., to a solution of methyl (2S,4R)-4-(difluoromethoxy)pyrrolidine-2-carboxylate (660 mg, 3.38 mmol) and (4-phenoxybenzoyl)glycine (917 mg, 3.38 mmol) in DMF (20 mL) was added T₃P (4.46 g, 10.15 mmol) and DIPEA (2.62 g, 20.29 mmol) under N₂ atmosphere and the mixture was stirred at room temperature overnight. The mixture was diluted with ethyl acetate and washed with saturated aq.NaHCO₃ solution. The aqueous layer was extracted with ethyl acetate twice and the combined organic layers were washed with brine, dried over anhydrous Na₂SO₄, filtered and concentrated to dryness under reduced pressure. The residue was purified by flash chromatography on silica gel (petroleum ether:ethyl acetate=1:1) to afford methyl (2S,4R)-4-(difluoromethoxy)-1-((4-phenoxybenzoyl)glycyl) pyrrolidine-2-carboxylate (1.5 g, 3.35 mmol, Yield 98.9%) as white solid. LC/MS (ESI) m/z: 449 (M+H)⁺.

### Step 2: (2S,4R)-4-(difluoromethoxy)-1-((4-phenoxybenzoyl)glycyl)pyrrolidine-2-carboxylic acid (3)

To a solution of methyl (2S,4R)-4-(difluoromethoxy)-1-((4-phenoxybenzoyl)glycyl) pyrrolidine-2-carboxylate (1.5 g, 3.35 mmol) in THF/MeOH/H₂O (v:v=4:1:1.18 mL) was added lithium hydroxide (281 mg, 6.69 mmol) under N₂ atmosphere and the reaction mixture was stirred at room temperature for 2 hours. The mixture was diluted with ethyl acetate and washed with H₂O. The aqueous layer was extracted with ethyl acetate twice and the combined organic layers were washed with brine, dried over anhydrous Na₂SO₄, filtered and concentrated to dryness under reduced pressure. The residue was purified by flash chromatography on silica gel (dichloromethane:methanol=10:1) to afford (2S,4R)-4-(difluoromethoxy)-1-((4-phenoxybenzoyl)glycyl)pyrrolidine-2-carboxylic acid (1.15 g, Yield 79.1%) as white solid. LC/MS (ESI) m/z:435 (M+H)⁺.

### Step 3: (2S,4R)-N-((S)-1-(4-carbamimidoylthiophen-2-yl)-2-methylpropyl)-4-(difluoromethoxy)-1-((4-phenoxybenzoyl)glycyl)pyrrolidine-2-carboxamide (Compound 188)

At 0° C., to a solution of (2S,4R)-4-(difluoromethoxy)-1-((4-phenoxybenzoyl)glycyl) pyrrolidine-2-carboxylic acid (77 mg, 0.18 mmol) and (R)-5-(1-amino-2-methylpropyl)thiophene-3-carboximidamide (35 mg, 0.18 mmol) in DMF (5 mL) was added DIPEA (138 mg, 1.06 mmol) and T₃P (339 mg, 0.53 mmol) under N₂ atmosphere and the reaction mixture was stirred at room temperature for 2 hours. The mixture was diluted with ethyl acetate and washed with saturated aq.NaHCO₃ solution. The aqueous layer was extracted with ethyl acetate twice and the combined organic layers were washed with brine, dried over anhydrous Na₂SO₄, filtered and concentrated to dryness under reduced pressure. The residue was purified by prep-HPLC to give Compound 188 (5 mg, Yield 4.6%) as a white solid. ¹H-NMR (400 MHz, CD₃OD) δ 8.51 (s, 1H), 8.24 (dd, J=9.4, 1.5 Hz, 1H), 7.87-7.80 (m, 2H), 7.43 (ddd, J=20.0, 9.4, 8.5 Hz, 3H), 7.20 (t, J=7.4 Hz, 1H), 7.07-6.98 (m, 4H), 6.50 (td, J=74.5, 14.0 Hz, 1H), 5.01-4.94 (m, 2H), 4.70 (dt, J=22.3, 7.6 Hz, 1H), 4.14 (dt, J=24.8, 16.7 Hz, 2H), 3.95-3.68 (m, 2H), 2.72-2.45 (m, 1H), 2.23 (ddt, J=20.6, 13.7, 7.4 Hz, 2H), 1.09-0.93 (m, 6H). LC/MS (ESI) m/z:614 (M+H)⁺.

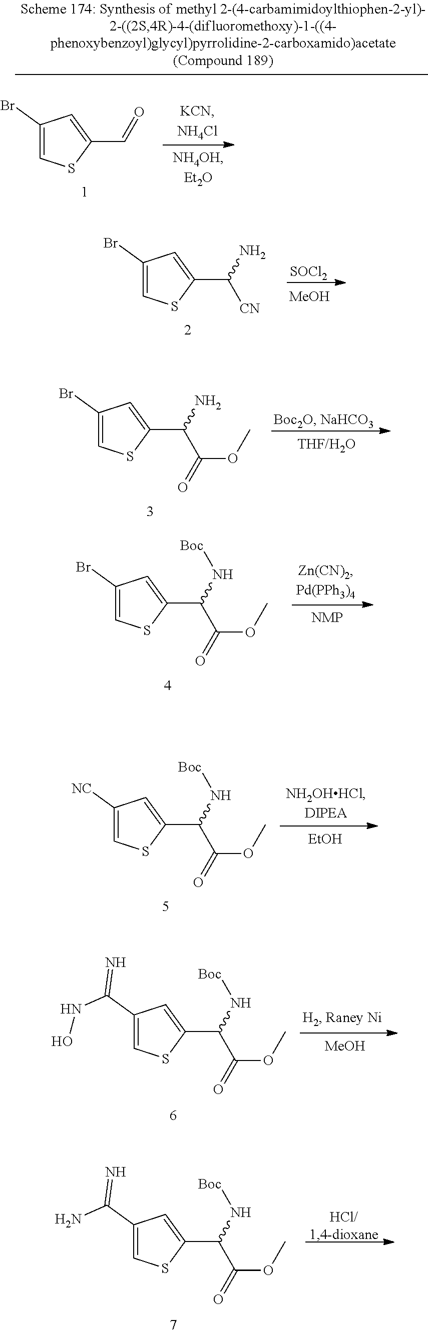

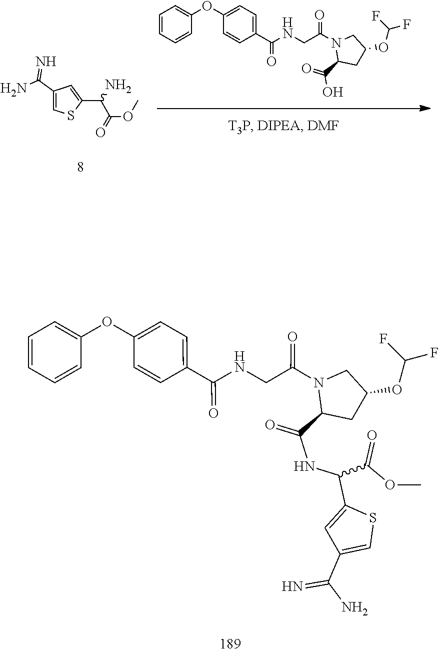 [SMILES: COC(=O)C(N)c1cc(C(=N)N)cs1 ; O=C(NCC(=O)N1C[C@H](OC(F)F)C[C@H]1C(=O)O)c1ccc(Oc2ccccc2)cc1 ; COC(=O)C(NC(=O)[C@@H]1C[C@@H](OC(F)F)CN1C(=O)CNC(=O)c1ccc(Oc2ccccc2)cc1)c1cc(C(=N)N)cs1]

### Step 1: 2-amino-2-(4-bromothiophen-2-yl)acetonitrile

To a mixture of NH₄Cl (13.87 g, 0.26 mol) and KCN (9.53 g, 0.15 mol) in NH₃·H₂O (200 mL) was added a solution of 4-bromothiophene-2-carbaldehyde (20 g, 0.11 mol) in Et₂O (200 mL) and the mixture was stirred at 60° C. for 16 hours in a sealed tube. The mixture was diluted with ice-water and extracted with ethyl acetate twice. The combined organic layers were washed with brine, dried over anhydrous Na₂SO₄, filtered and concentrated to dryness under reduced pressure. The residue was purified by flash chromatography on silica gel (petroleum ether:ethyl acetate=100:1 to 1:1) to give 2-amino-2-(4-bromothiophen-2-yl)acetonitrile (13.0 g, yield 57.5%) as yellow oil. LC/MS (ESI) (m/z): 200/202(M−NH₂)⁺.

### Step 2: methyl 2-amino-2-(4-bromothiophen-2-yl)acetate

To a solution of SOCl₂ (13.1 mL, 0.18 mol) in MeOH (200 mL) was drop-wise added a solution of 2-amino-2-(4-bromothiophen-2-yl)acetonitrile (13 g, 0.06 mol) in MeOH (60 mL) and the reaction mixture was stirred at 90° C. for 16 hours in a sealed tube. The mixture was concentrated to dryness under reduced pressure. The residue was acidified with 1.0 N aq.HCl to pH-4 and extracted with ethyl acetate twice. The aqueous layer was basified by aq.Na₂CO₃ solution to pH-8 and extracted with ethyl acetate twice, the combined organic layers were washed with brine, dried over anhydrous Na₂SO₄, filtered and concentrated to dryness to give methyl 2-amino-2-(4-bromothiophen-2-yl)acetate (7.8 g, yield 52.2%) as yellow oil, which was used directly in the next step without further purification. LC/MS (ESI) (m/z): 233/235(M−NH₂)⁺.

### Step 3: methyl 2-(4-bromothiophen-2-yl)-2-((tert-butoxycarbonyl)amino)acetate

To a mixture of methyl 2-amino-2-(4-bromothiophen-2-yl)acetate (7.8 g, 31.33 mmol) and (Boc)₂O (20.49 g, 93.98 mmol) in THE (60 mL) and water (60 mL) was added NaHCO₃ (26.32 g, 313.3 mmol) at 0° C. and the mixture was stirred at 25° C. for 16 hours. The mixture was diluted with water and extracted with ethyl acetate twice. The combined organic layers were washed with brine, dried over anhydrous Na₂SO₄, filtered and concentrated to dryness under reduced pressure. The residue was purified by flash chromatography on silica gel (petroleum ether:ethyl acetate=20:1) to give methyl 2-(4-bromothiophen-2-yl)-2-((tert-butoxycarbonyl)amino)acetate (10.9 g, yield 99.7%) as yellow oil. LC/MS (ESI) (m/z): 350/352(M+H)⁺.

### Step 4: methyl 2-((tert-butoxycarbonyl)amino)-2-(4-cyanothiophen-2-yl)acetate

To a solution of methyl 2-(4-bromothiophen-2-yl)-2-((tert-butoxycarbonyl)amino)acetate (10.9 g, 31.23 mmol) in DMF (160 mL) was added Zn(CN)₂ (10.96 g, 93.70 mmol) and Pd(PPh₃)₄ (5.415 g, 4.68 mmol). The reaction mixture was stirred at 90° C. for 6 hours under N₂ atmosphere. The mixture was diluted with ethyl acetate and washed with water. The aqueous layer was extracted with ethyl acetate twice and the combined organic layers were washed with brine, dried over anhydrous Na₂SO₄, filtered and concentrated to dryness under reduced pressure. The residue was purified by flash chromatography on silica gel (petroleum ether:ethyl acetate=50:1) to give methyl 2-((tert-butoxycarbonyl)amino)-2-(4-cyanothiophen-2-yl)acetate (7.6 g, yield 82.2%) as light oil. LC/MS (ESI) m/z: 297 (M+H)⁺.

### Step 5: methyl 2-((tert-butoxycarbonyl)amino)-2-(4-(N-hydroxycarbamimidoyl)thiophen-2-yl)acetate

To a solution of methyl 2-((tert-butoxycarbonyl)amino)-2-(4-cyanothiophen-2-yl)acetate (800 mg, 2.70 mmol) in EtOH (10 mL) was added NH₂OH hydrochloride salt (469 mg, 6.76 mmol) and DIPEA (1.34 mL, 8.10 mmol), the reaction mixture was stirred at 25° C. for 16 hours. The mixture was diluted with DCM and washed with water. The aqueous layer was extracted with DCM twice and the combined organic layers were washed with brine, dried over anhydrous Na₂SO₄, filtered and concentrated to dryness under reduced pressure. The residue was purified by flash chromatography on silica gel (petroleum ether:ethyl acetate=2:1) to give methyl 2-((tert-butoxycarbonyl)amino)-2-(4-(N-hydroxycarbamimidoyl)thiophen-2-yl)acetate (460 mg, yield 51.9%) as light oil. LC/MS (ESI) m/z:330 (M+H)⁺.

### Step 6: methyl 2-((tert-butoxycarbonyl)amino)-2-(4-carbamimidoylthiophen-2-yl)acetate

To a solution of methyl 2-((tert-butoxycarbonyl)amino)-2-(4-(N-hydroxycarbamimidoyl)thiophen-2-yl)acetate (460 mg, 1.40 mmol) in methanol (3 mL) was added Raney Nickel (50 mg) and AcOH (one drop), and the reaction mixture was stirred under H₂ atmosphere at 30° C. for 16 hours. The mixture was filtered and the filtrate was concentrated to dryness under reduced pressure to give methyl 2-((tert-butoxycarbonyl)amino)-2-(4-carbamimidoylthiophen-2-yl)acetate (430 mg, yield 98.34%) as yellow oil. LC/MS (ESI) m/z:314 (M+H)⁺.

### Step 7: methyl 2-amino-2-(4-carbamimidoylthiophen-2-yl)acetate

A solution of methyl 2-((tert-butoxycarbonyl)amino)-2-(4-carbamimidoylthiophen-2-yl) acetate (100 mg, 0.32 mmol) was stirred in HCl/1,4-dioxane (3 mL, 4M) under N₂ atmosphere at room temperature for 2 hours. The mixture was concentrated to dryness, co-evaporated with dichloromethane twice and dried under vacuum to give methyl 2-amino-2-(4-carbamimidoylthiophen-2-yl)acetate (60 mg, yield 88.2%) as white solid. LC/MS (ESI) m/z: 214 (M+H)⁺.

### Step 8: methyl 2-(4-carbamimidoylthiophen-2-yl)-2-((2S,4R)-4-(difluoromethoxy)-1-((4-phenoxybenzoyl)glycyl)pyrrolidine-2-carboxamido)acetate (Compound 189)

To a mixture of methyl 2-amino-2-(4-carbamimidoylthiophen-2-yl)acetate (60 mg, 0.28 mmol) and (2S,4R)-4-(difluoromethoxy)-1-((4-phenoxybenzoyl)glycyl)pyrrolidine-2-carboxylic acid (97 mg, 0.23 mmol) in DMF (3 mL) was added DIPEA (180 mg, 1.40 mmol) and T₃P (133 mg, 0.42 mmol, 50% wt. in ethyl acetate) at 0° C. under N₂ atmosphere and the mixture was stirred at room temperature for 1 hour. The mixture was diluted with ethyl acetate and washed with saturated aq.NaHCO₃ solution. The aqueous layer was extracted with ethyl acetate twice and the combined organic layers were washed with brine, dried over anhydrous Na₂SO₄, filtered and concentrated to dryness under reduced pressure. The residue was purified by chromatography on silica gel (dichloromethane:methanol=10:1) and further purified by pre-HPLC to give Compound 189 (1.6 mg, yield 0.9%) as white solid. ¹H NMR (400 MHz, CD₃OD) δ 8.38-8.28 (m, 1H), 7.89-7.79 (m, 2H), 7.68-7.54 (m, 1H), 7.46-7.38 (m, 2H), 7.24-7.18 (m, 1H), 7.11-7.04 (m, 2H), 7.04-6.96 (m, 2H), 6.57-6.27 (m, 1H), 5.04-4.99 (m, 1H), 4.73-4.57 (m, 1H), 4.32-4.10 (m, 2H), 4.08-3.45 (m, 6H), 2.56-2.43 (m, 1H), 2.25 (m, 1H). LC/MS (ESI) m/z:630 (M+H)⁺.

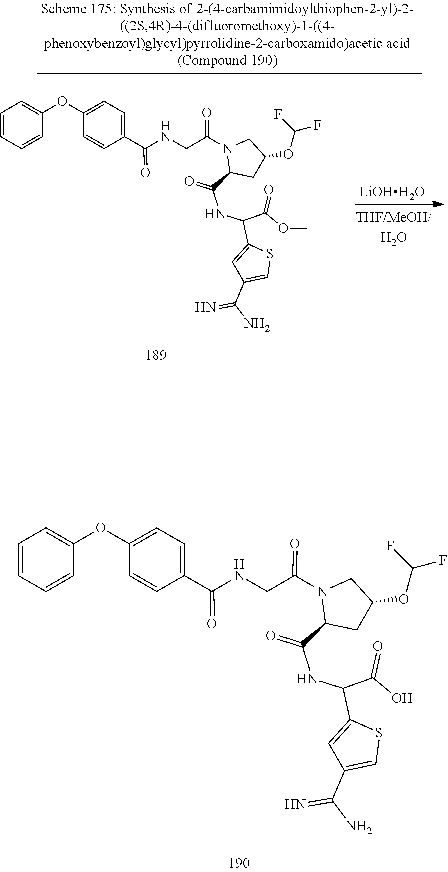 [SMILES: COC(=O)C(NC(=O)[C@@H]1C[C@@H](OC(F)F)CN1C(=O)CNC(=O)c1ccc(Oc2ccccc2)cc1)c1cc(C(=N)N)cs1 ; "LiOH·H2O" ; N=C(N)c1csc(C(NC(=O)[C@@H]2C[C@@H](OC(F)F)CN2C(=O)CNC(=O)c2ccc(Oc3ccccc3)cc2)C(=O)O)c1]

### Step 1: 2-(4-carbamimidoylthiophen-2-yl)-2-((2S,4R)-4-(difluoromethoxy)-1-((4-phenoxybenzoyl)glycyl)pyrrolidine-2-carboxamido)acetic acid

To a solution of Compound 189 (40 mg, 0.064 mmol) in methanol (1.4 mL) and tetrahydrofuran (0.7 mL) was added a solution of lithium hydroxide (5 mg, 0.13 mmol) in water (0.7 mL) and the mixture was stirred at 25° C. for 2 hours. The mixture was acidified with 1.0N hydrochloric acid solution to pH-5 and extracted with ethyl acetate twice. The aqueous layer was purified prep-HPLC to give Compound 190 (2.2 mg, yield 5.6%) as a white solid. ¹H NMR (400 MHz, CD₃OD) δ 8.23-8.13 (m, 1H), 7.88-7.80 (m, 2H), 7.58-7.38 (m, 3H), 7.20 (t, J=7.4 Hz, 1H), 7.08-7.04 (m, 2H), 7.02-6.96 (m, 2H), 6.71-6.24 (m, 1H), 5.63-5.43 (m, 1H), 5.05-4.95 (m, 1H), 4.76-4.67 (m, 1H), 4.36-4.08 (m, 2H), 4.04-3.83 (m, 2H), 2.55-2.41 (m, 1H), 2.40-2.25 (m, 1H). LC/MS (ESI) (m/z): 616 (M+H)⁺.

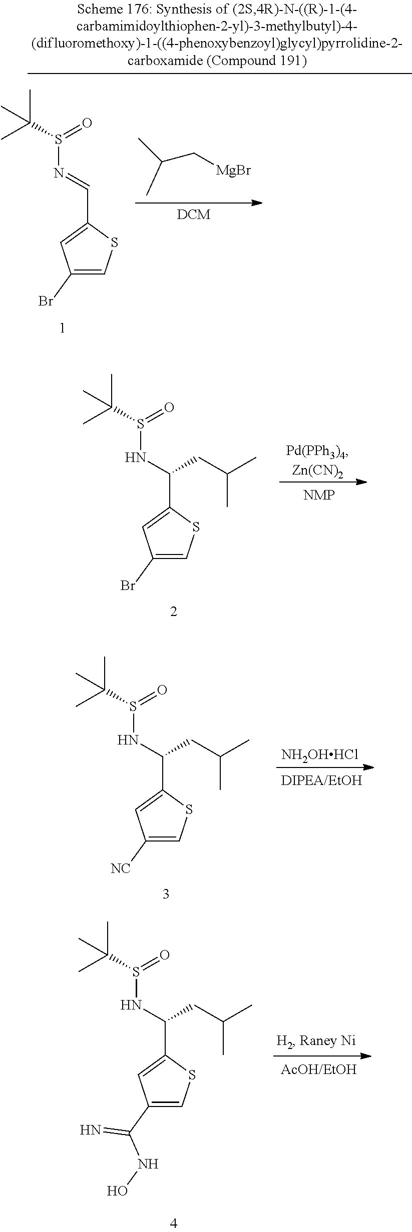

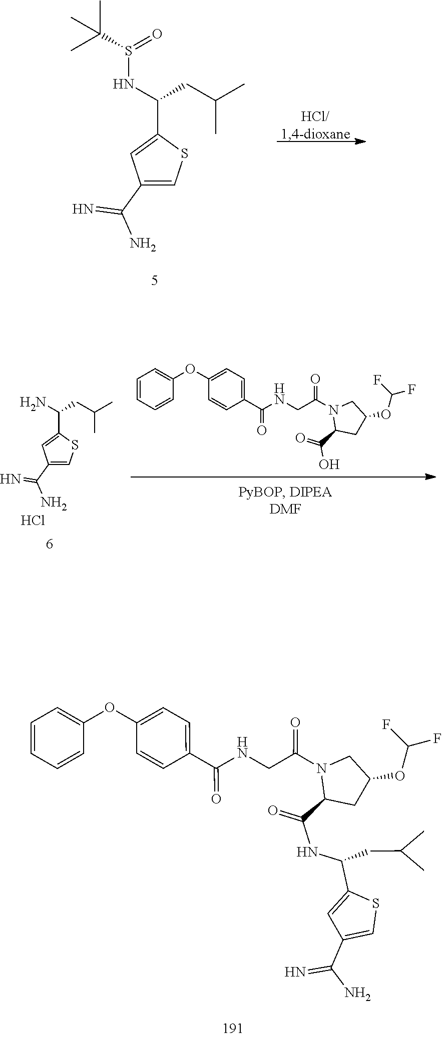

### Step 1: (S)-N-((R)-1-(4-bromothiophen-2-yl)-3-methylbutyl)-2-methylpropane-2-sulfinamide

At −78° C., to a solution of (S,E)-N-((4-bromothiophen-2-yl)methylene)-2-methylpropane-2-sulfinamide (1.5 g, 5.12 mmol) in THE (15 mL) was added isobutyl magnesium bromide (10.24 mL, 10.24 mmol) drop-wisely under N₂ atmosphere and the reaction mixture was stirred at −78° C. for 2 hours. The mixture was quenched with saturated aq.NH₄Cl solution and extracted with EtOAc twice. The combined organic layers were washed with brine, dried over anhydrous Na₂SO₄, filtered and concentrated under reduced pressure to dryness. The residue was purified by flash chromatography on silica gel (PE:EtOAc=10:1) to afford (S)-N-((R)-1-(4-bromothiophen-2-yl)-3-methylbutyl)-2-methylpropane-2-sulfinamide (407 mg, yield 23.9%) as light oil. LC/MS (ESI) m/z: 352 (M+H)⁺.

### Step 2: (S)-N-((R)-1-(4-cyanothiophen-2-yl)-3-methylbutyl)-2-methylpropane-2-sulfinamide

To a solution of (S)-N-((R)-1-(4-bromothiophen-2-yl)-3-methylbutyl)-2-methylpropane-2-sulfinamide (400 mg, 1.14 mmol) in NMP (5 mL) was added Zn(CN)₂ (799.7 mg, 2.27 mmol) and Pd(PPh₃)₄ (197 mg, 0.17 mmol). The reaction mixture was degassed under N₂ atmosphere for three times and stirred at 160° C. for 1 hour. The mixture was diluted with EtOAc, washed with water and brine, dried over anhydrous Na₂SO₄, filtered and concentrated under reduced pressure to dryness. The residue was purified by flash chromatography on silica gel (PE:EtOAc=1:1) to afford (S)-N-((R)-1-(4-cyanothiophen-2-yl)-3-methylbutyl)-2-methylpropane-2-sulfinamide (215 mg, yield 63.3%) as light yellow oil. LC/MS (ESI) m/z: 299 (M+H)⁺.

### Step 3: 5-((R)-1-(((S)-tert-butylsulfinyl)amino)-3-methylbutyl)-N-hydroxythiophene-3-carboximidamide

To a solution of (S)-N-((R)-1-(4-cyanothiophen-2-yl)-3-methylbutyl)-2-methylpropane-2-sulfinamide (210 mg, 0.70 mmol) in EtOH (3 mL) was added DIPEA (271 mg, 2.12 mmol) and NH₂OH·HCl (112.9 mg, 1.75 mmol) and the mixture was stirred at 25° C. for 16 hours. The reaction mixture was diluted with EtOAc, washed with water and brine, dried over anhydrous Na₂SO₄, filtered and concentrated to dryness. The residue was purified by chromatography on silica gel (DCM:MeOH=10:1) to give 5-((R)-1-(((S)-tert-butylsulfinyl)amino)-3-methylbutyl)-N-hydroxythiophene-3-carboximidamide (46 mg, yield 19.7%) as colorless oil. LC/MS (ESI) m/z: 332 (M+H)⁺.

### Step 4: 5-((R)-1-(((S)-tert-butylsulfinyl)amino)-3-methylbutyl)thiophene-3-carboximidamide

To a solution of 5-((R)-1-(((S)-tert-butylsulfinyl)amino)-3-methylbutyl)-N-hydroxythiophene-3-carboximidamide (150 mg, 0.45 mmol) in MeOH (2 mL) and AcOH (0.02 mL) was added Raney Nickel (10 mg) at 0° C., and the mixture was degassed under N₂ atmosphere for three times and stirred under a H₂ balloon at room temperature overnight. The mixture was filtered and the filtrate was concentrated under reduced pressure to give 5-((R)-1-(((S)-tert-butylsulfinyl)amino)-3-methylbutyl)thiophene-3-carboximidamide (140 mg, yield 98%) as yellow oil, which was used directly in the next step without further purification. LC/MS (ESI) (m/z): 316 (M+H)⁺.

### Step 5: (R)-5-(1-amino-3-methylbutyl)thiophene-3-carboximidamide hydrochloride

A mixture of 5-((R)-1-(((S)-tert-butylsulfinyl)amino)-3-methylbutyl)thiophene-3-carboximidamide (140 mg, 0.44 mmol) in HCl/1,4-dioxane (3 mL, 4M) was stirred at room temperature for 1 hour. The reaction mixture was concentrated to dryness under reduced pressure, co-evaporated with DCM twice and dried under vacuum to give (R)-5-(1-amino-3-methylbutyl)thiophene-3-carboximidamide hydrochloride (120 mg, yield 100%) as white solid, which was used directly in the next reaction without further purification. LC/MS (ESI) m/z: 212 (M+H)⁺.

### Step 6: (2S,4R)-N-((R)-1-(4-carbamimidoylthiophen-2-yl)-3-methylbutyl)-4-(difluoromethoxy)-1-((4-phenoxybenzoyl)glycyl)pyrrolidine-2-carboxamide (Compound 191)

To a mixture of (R)-5-(1-amino-3-methylbutyl)thiophene-3-carboximidamide hydrochloride (47 mg, 0.19 mmol) and (2S,4R)-4-(difluoromethoxy)-1-((4-phenoxybenzoyl)glycyl)pyrrolidine-2-carboxylic acid (66 mg, 0.15 mmol) in DMF (2 mL) was added DIPEA (0.157 mL, 0.95 mmol) and PyBOP (148 mg, 0.28 mmol) at 0° C. under N₂ atmosphere and the mixture was stirred at 25° C. for 2 hours. The mixture was diluted with EtOAc, washed with water and brine, dried over anhydrous Na₂SO₄, filtered and concentrated under reduced pressure to dryness. The residue was purified by prep-TLC (DCM:MeOH=10:1) and further purified by prep-HPLC to give Compound 191 (1.4 mg, yield 1.19%) as a white solid. ¹H NMR (400 MHz, CD₃OD) δ 8.55 (s, 1H), 8.21 (s, 1H), 7.85 (t, J=7.5 Hz, 2H), 7.52 (d, J=14.2 Hz, 1H), 7.41 (t, J=8.0 Hz, 2H), 7.20 (t, J=7.4 Hz, 1H), 7.05 (d, J=7.7 Hz, 2H), 7.00 (d, J=8.8 Hz, 2H), 6.50 (dd, J=81.9, 66.9 Hz, 1H), 5.35-5.22 (m, 1H), 5.01 (dd, J=7.9, 3.2 Hz, 1H), 4.58 (t, J=7.9 Hz, 1H), 4.21 (s, 1H), 4.14 (d, J=16.6 Hz, 1H), 3.95 (dd, J=11.4, 4.5 Hz, 1H), 3.87 (d, J=11.1 Hz, 1H), 2.47 (d, J=8.6 Hz, 1H), 2.20 (dd, J=12.2, 6.9 Hz, 1H), 1.91-1.82 (m, 1H), 1.73-1.66 (m, 2H), 1.01-0.93 (m, 6H). LC/MS (ESI) (m/z): 628 (M+H)⁺.

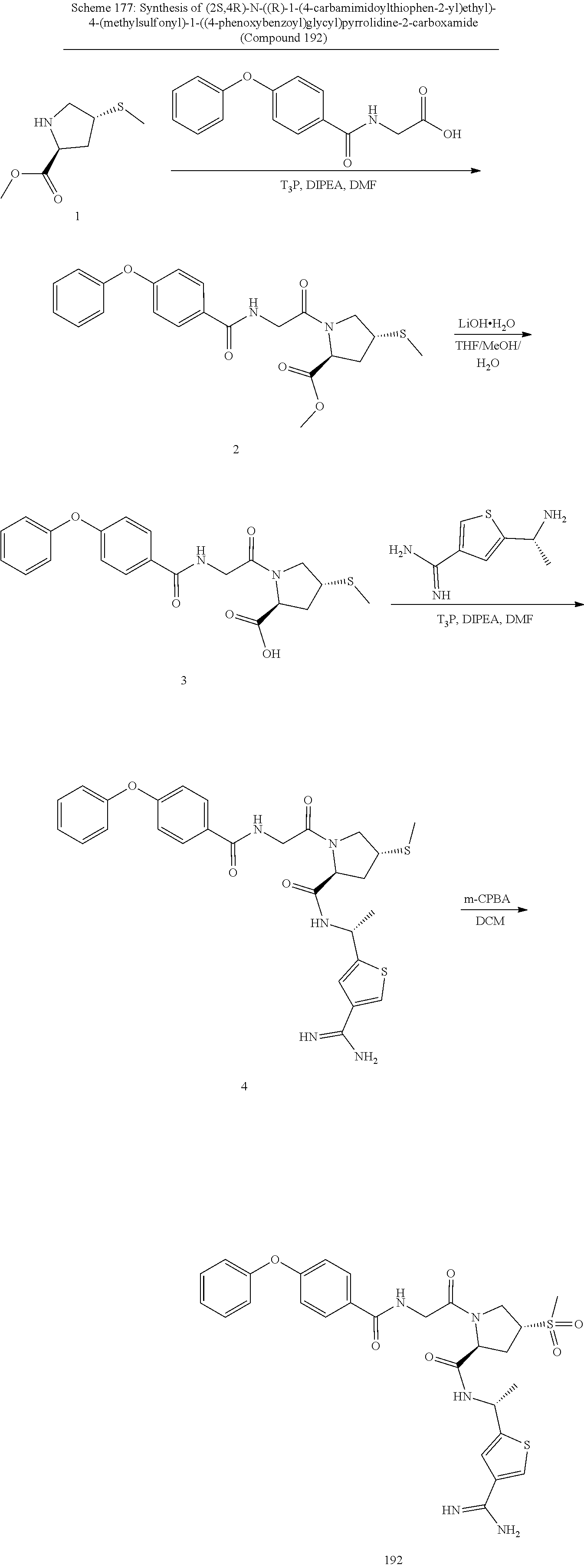

### Step 1: methyl (2S,4R)-4-(methylthio)-1-((4-phenoxybenzoyl)glycyl)pyrrolidine-2-carboxylate (2)

To a mixture of methyl (2S,4R)-4-(methylthio)pyrrolidine-2-carboxylate (125 mg, 0.71 mmol) and (4-phenoxybenzoyl)glycine (193 mg, 0.71 mmol) in DMF (10 mL) was added DIPEA (553 mg, 4.28 mmol) and T₃P (1.4 g, 2.14 mmol, 50% wt. in EtOAc) under N₂ atmosphere and the mixture was stirred at room temperature overnight. The mixture was diluted with EtOAc and washed with saturated aq.NaHCO₃ solution. The aqueous layer was extracted with EtOAc twice and the combined organic layers were washed with brine, dried over anhydrous Na₂SO₄, filtered and concentrated to dryness under reduced pressure. The residue was purified by flash chromatography on silica gel (PE:EtOAc=1:1) to afford methyl (2S,4R)-4-(methylthio)-1-((4-phenoxybenzoyl)glycyl)pyrrolidine-2-carboxylate (210 mg, yield 68.7%) as white solid. LC/MS (ESI) m/z: 429 (M+H)⁺.

### Step 2: (2S,4R)-4-(methylthio)-1-((4-phenoxybenzoyl)glycyl)pyrrolidine-2-carboxylic acid (3)

To a solution of methyl (2S,4R)-4-(methylthio)-1-((4-phenoxybenzoyl)glycyl)pyrrolidine-2-carboxylate (210 mg, 0.49 mmol) in THF/MeOH/H₂O (3 mL, v:v=4/1/1) was added LiOH (41 mg, 0.98 mmol) and the reaction mixture was stirred at room temperature for 2 hours. The mixture was concentrated to dryness and the residue was dissolved in water (5 mL). The mixture was washed with EtOAc, acidified with 1N aq.HCl to pH-4 and extracted with DCM twice. The combined organic layers were washed with brine, dried over anhydrous Na₂SO₄, filtered and concentrated to dryness under reduced pressure. The residue was purified by flash chromatography on silica gel (DCM:MeOH=10:1) to afford (2S,4R)-4-(methylthio)-1-((4-phenoxybenzoyl)glycyl)pyrrolidine-2-carboxylic acid (200 mg, yield 98.5%) as white solid. LC/MS (ESI) m/z: 415 (M+H)⁺.

### Step 3: (2S,4R)-N-((R)-1-(4-carbamimidoylthiophen-2-yl)ethyl)-4-(methylthio)-1-((4-phenoxybenzoyl)glycyl)pyrrolidine-2-carboxamide (4)

To a mixture of (2S,4R)-4-(methylthio)-1-((4-phenoxybenzoyl)glycyl)pyrrolidine-2-carboxylic acid (200 mg, 0.48 mmol) and (R)-5-(1-aminoethyl)thiophene-3-carboximidamide (82 mg, 0.48 mmol) in DMF (5 mL) was added DIPEA (374 mg, 2.90 mmol) and T₃P (461 mg, 0.72 mmol, 50% wt. in EtOAc) under N₂ atmosphere and the mixture was stirred at room temperature overnight. The mixture was diluted with EtOAc and washed with saturated aq.NaHCO₃ solution. The aqueous layer was extracted with EtOAc twice and the combined organic layers were washed with brine, dried over anhydrous Na₂SO₄, filtered and concentrated to dryness under reduced pressure. The residue was purified by flash chromatography on silica gel (PE:EtOAc=1:1) to afford (2S,4R)-N-((R)-1-(4-carbamimidoylthiophen-2-yl)ethyl)-4-(methylthio)-1-((4-phenoxybenzoyl) glycyl)pyrrolidine-2-carboxamide (40 mg, yield 14.7%) as white solid. LC/MS (ESI) m/z: 566 (M+H)⁺.

### Step 4: (2S,4R)-N-((R)-1-(4-carbamimidoylthiophen-2-yl)ethyl)-4-(methylsulfonyl)-1-((4-phenoxybenzoyl)glycyl)pyrrolidine-2-carboxamide (Compound 192)

To a solution of (2S,4R)-N-((R)-1-(4-carbamimidoylthiophen-2-yl)ethyl)-4-(methylthio)-1-((4-phenoxybenzoyl)glycyl)pyrrolidine-2-carboxamide (20 mg, 0.035 mmol) in DCM (2 mL) was added m-CPBA (23 mg, 0.11 mmol) under N₂ atmosphere and the reaction mixture was stirred at room temperature for 3 hours. The mixture was diluted with EtOAc and washed with saturated aq.NaHCO₃ solution. The aqueous layer was extracted with EtOAc twice and the combined organic layers were washed with brine, dried over anhydrous Na₂SO₄, filtered and concentrated to dryness under reduced pressure. The residue was purified by prep-HPLC to give Compound 192 (8 mg, yield 37.9%) as a white solid. ¹H NMR (400 MHz, CD₃OD) δ 8.53 (s, 1H), 8.23 (dd, J=10.2, 1.5 Hz, 1H), 7.84 (dd, J=8.8, 6.9 Hz, 2H), 7.52 (d, J=22.7 Hz, 1H), 7.42 (t, J=8.0 Hz, 2H), 7.21 (t, J=7.4 Hz, 1H), 7.08-6.99 (m, 4H), 5.37-5.22 (m, 1H), 4.78-4.67 (m, 1H), 4.27-4.12 (m, 4H), 4.00 (ddd, J=20.2, 15.1, 7.9 Hz, 1H), 3.06 (d, J=17.9 Hz, 3H), 2.94-2.69 (m, 1H), 2.60-2.36 (m, 1H), 1.62 (dd, J=29.9, 7.0 Hz, 3H). LC/MS (ESI) m/z: 598 (M+H)⁺.

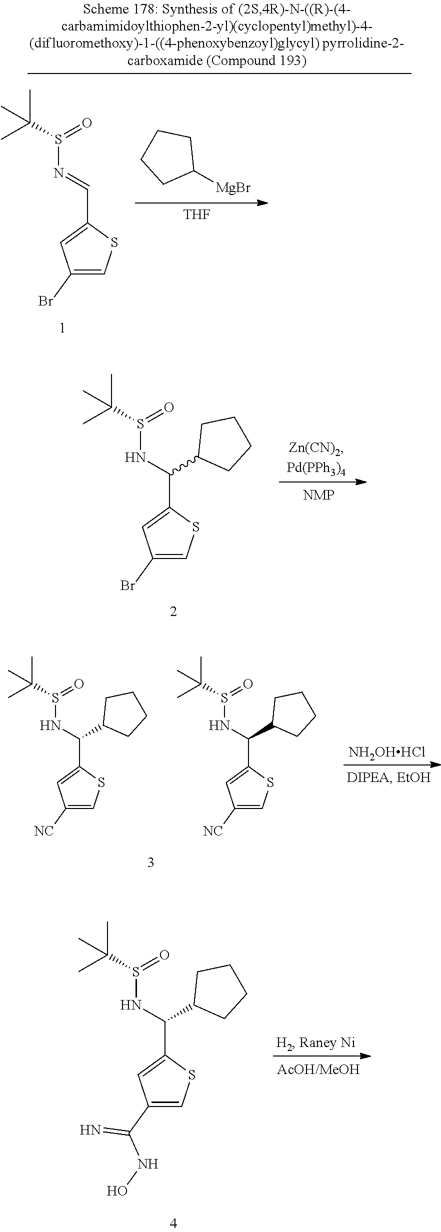

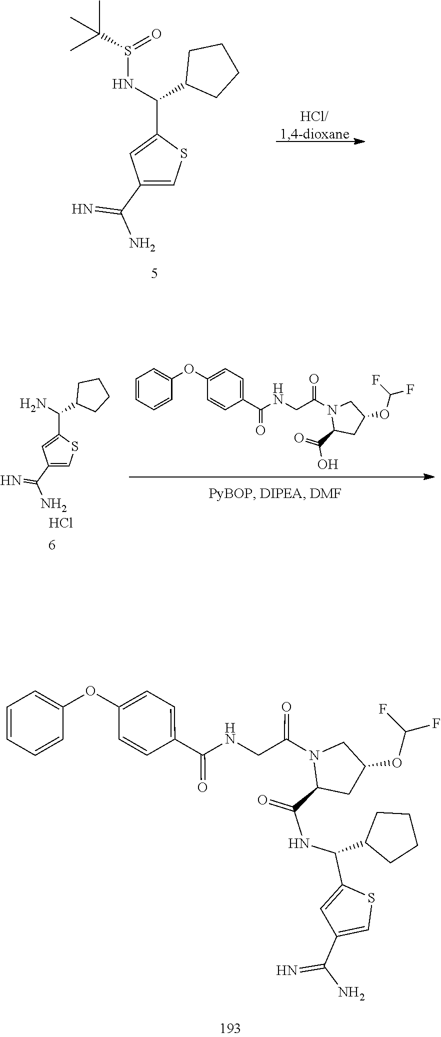

### Step 1: (S)-N-((4-bromothiophen-2-yl)(cyclopentyl)methyl)-2-methylpropane-2-sulfinamide (2)

At −78° C., to a solution of (S,E)-N-((4-bromothiophen-2-yl)methylene)-2-methylpropane-2-sulfinamide (1.5 g, 5.1 mmol) in THE (15 mL) was added cyclopentyl magnesium bromide (10.2 mL, 10.2 mmol) drop-wisely under N₂ atmosphere and the reaction mixture was stirred at −78° C. for 2 hours. The mixture was quenched with saturated aq.NH₄Cl solution and extracted with EtOAc twice. The combined organic layers were washed with brine, dried over anhydrous Na₂SO₄, filtered and concentrated under reduced pressure to dryness. The residue was purified by flash chromatography on silica gel (petroleum ether:EtOAc=80:1) to afford (S)-N-((4-bromothiophen-2-yl)(cyclopentyl)methyl)-2-methylpropane-2-sulfinamide (812 mg, yield 43.7%) as yellow oil. LC/MS (ESI) m/z: 363 (M+H)⁺.

### Step 2: (S)-N-((R)-(4-cyanothiophen-2-yl)(cyclopentyl)methyl)-2-methylpropane-2-sulfinamide (3)

To a solution of (S)-N-((4-bromothiophen-2-yl)(cyclopentyl)methyl)-2-methylpropane-2-sulfinamide (810 mg, 2.23 mmol) in NMP (10 mL) was added Zn(CN)₂ (1.59 g, 4.46 mmol) and Pd(PPh₃)₄ (387 mg, 0.335 mmol). The reaction mixture was degassed under N₂ atmosphere for three times and stirred at 160° C. for 1 hour. The mixture was diluted with EtOAc, washed with water and brine, dried over anhydrous Na₂SO₄, filtered and concentrated under reduced pressure to dryness. The residue was purified by flash chromatography on silica gel (DCM:MeOH=600:1) to afford (S)-N-((R)-(4-cyanothiophen-2-yl)(cyclopentyl)methyl)-2-methylpropane-2-sulfinamide (238 mg, yield 28.7%) as yellow oil. LC/MS (ESI) m/z: 311 (M+H)⁺.

### Step 3: 5-((R)-(((S)-tert-butylsulfinyl)amino)(cyclopentyl)methyl)-N-hydroxythiophene-3-carboximidamide (4)

To a mixture of (S)-N-((R)-(4-cyanothiophen-2-yl)(cyclopentyl)methyl)-2-methylpropane-2-sulfinamide (200 mg, 0.655 mmol) in EtOH (3 mL) was added DIPEA (0.324 mL, 1.965 mmol) and NH₂OH·HCl (106 mg, 1.64 mmol) and the mixture was stirred at 25° C. for 16 hours. The mixture was diluted with EtOAc, washed with water and brine, dried over anhydrous Na₂SO₄, filtered and concentrated to dryness. The residue was purified by chromatography on silica gel (PE:EtOAc=1:2) to give 5-((R)-(((S)-tert-butylsulfinyl)amino)(cyclopentyl)methyl)-N-hydroxythiophene-3-carboximidamide (117 mg, yield 53%) as colorless oil. LC/MS (ESI) m/z: 344 (M+H)⁺.

### Step 4: 5-((R)-(((S)-tert-butylsulfinyl)amino)(cyclopentyl)methyl)thiophene-3-carboximidamide (5)

To a solution of 5-((R)-(((S)-tert-butylsulfinyl)amino)(cyclopentyl)methyl)-N-hydroxythiophene-3-carboximidamide (110 mg, 0.32 mmol) in MeOH (2 mL) and AcOH (0.02 ml) was added Raney Ni (10 mg) at 0° C., and the mixture was degassed under N₂ atmosphere for three times and stirred under a H₂ balloon at room temperature overnight. The mixture was filtered and the filtrate was concentrated under reduce pressure to give 5-((R)-(((S)-tert-butylsulfinyl)amino)(cyclopentyl)methyl)thiophene-3-carboximidamide (90 mg, yield 86.5%) as colorless oil, which was used directly in the next reaction without further purification. LC/MS (ESI) (m/z): 328 (M+H)⁺.

### Step 5: (R)-5-(amino(cyclopentyl)methyl)thiophene-3-carboximidamide hydrochloride (6)

A mixture of 5-((R)-(((S)-tert-butylsulfinyl)amino)(cyclopentyl)methyl)thiophene-3-carboximidamide (90 mg, 0.28 mmol) in HCl/1,4-dioxane (2 mL, 4M) was stirred at room temperature for 1 hour. The reaction mixture was concentrated to dryness under reduced pressure, co-evaporated with DCM and dried under vacuum to give (R)-5-(amino(cyclopentyl)methyl)thiophene-3-carboximidamide hydrochloride (70 mg, yield 98.9%) as white solid, which was used directly in the next reaction without further purification. LC/MS (ESI) m/z: 224 (M+H)⁺.

### Step 6: (2S,4R)-N-((R)-(4-carbamimidoylthiophen-2-yl)(cyclopentyl)methyl)-4-(difluoromethoxy)-1-((4-phenoxybenzoyl)glycyl)pyrrolidine-2-carboxamide (Compound 193)

To a mixture of (R)-5-(amino(cyclopentyl)methyl)thiophene-3-carboximidamide (30 mg, 0.15 mmol) and (2S,4R)-4-(difluoromethoxy)-1-((4-phenoxybenzoyl)glycyl)pyrrolidine-2-carboxylic acid (52 mg, 0.14 mmol) in DMF (2 mL) was added DIPEA (97 mg, 0.75 mmol) and PyBOP (117 mg, 0.225 mmol) at 0° C. under N₂ atmosphere and the mixture was stirred at 25° C. for 3 hours. The mixture was quenched with water and extracted with EtOAc twice. The combined organic layers were washed with brine, dried over anhydrous Na₂SO₄, filtered and concentrated to dryness under reduced pressure. The residue was purified by prep-TLC (DCM:MeOH=10:1) and further purified by prep-HPLC to give Compound 193 (1.8 mg, yield 2.1%) as a white solid. ¹H NMR (400 MHz, CD₃OD) δ 8.53 (s, 1H), 8.23 (dd, J=5.2, 1.4 Hz, 1H), 7.84 (t, J=9.2 Hz, 2H), 7.50 (d, J=20.7 Hz, 1H), 7.42 (d, J=7.7 Hz, 2H), 7.20 (t, J=7.3 Hz, 1H), 7.05 (d, J=7.8 Hz, 2H), 7.00 (d, J=8.8 Hz, 2H), 6.69-6.32 (m, 1H), 4.96 (d, J=9.6 Hz, 2H), 4.59 (t, J=7.8 Hz, 1H), 4.22-4.19 (m, 1H), 4.14 (d, J=16.6 Hz, 1H), 3.96-3.92 (m, 1H), 3.86 (d, J=12.2 Hz, 1H), 2.43 (dd, J=15.2, 8.5 Hz, 2H), 2.22-2.15 (m, 1H), 1.89-1.82 (m, 1H), 1.73-1.61 (m, 5H), 1.59-1.53 (m, 2H). LC/MS (ESI) (m/z): 586 (M+H)⁺.

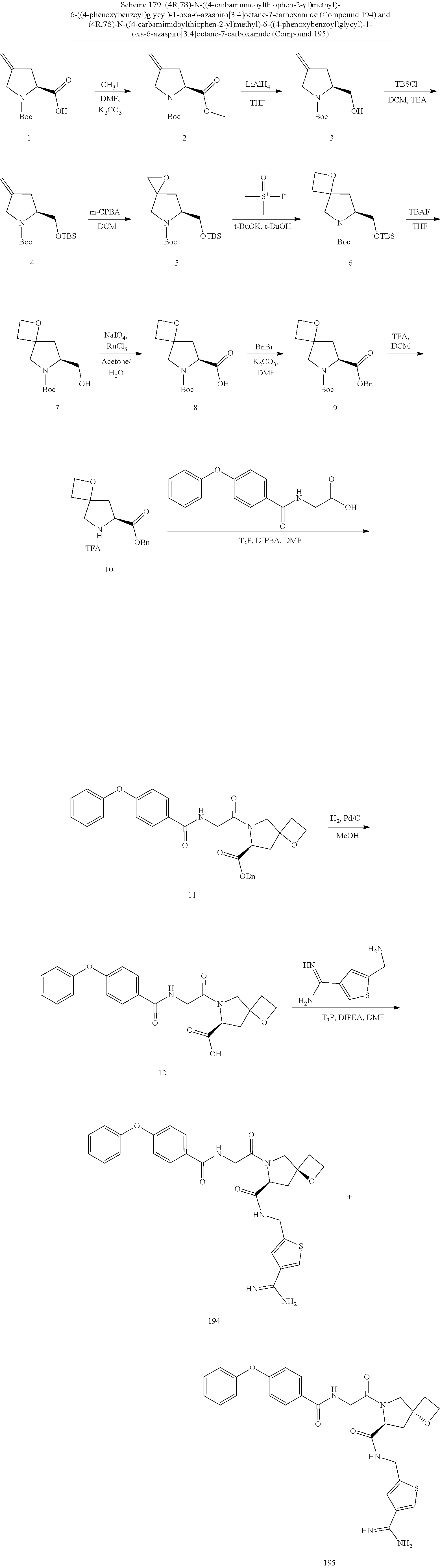

### Step 1: 1-(tert-butyl) 2-methyl (S)-4-methylenepyrrolidine-1,2-dicarboxylate (2)

To a solution of (S)-1-(tert-butoxycarbonyl)-4-methylenepyrrolidine-2-carboxylic acid (1.0 g, 4.4 mmol) in DMF (10 mL) was added K₂CO₃ (2.43 g, 17.6 mmol) and iodomethane (0.55 mL, 8.8 mmol) and the mixture was stirred at 25° C. for 16 hours. The mixture was diluted with EtOAc, washed with water and brine, dried over anhydrous Na₂SO₄, filtered and concentrated under reduced pressure to dryness. The residue was purified by column chromatography on silica gel (PE:EtOAc=10:1) to give 1-(tert-butyl) 2-methyl (S)-4-methylenepyrrolidine-1,2-dicarboxylate (1.06 g, yield 100%) as colorless oil. LC/MS (ESI) m/z: 242 (M+H)⁺.

### Step 2: tert-butyl (S)-2-(hydroxymethyl)-4-methylenepyrrolidine-1-carboxylate (3)

At 0° C., to a solution of 1-(tert-butyl) 2-methyl (S)-4-methylenepyrrolidine-1,2-dicarboxylate (4.67 g, 19.4 mmol) in THE (50 mL) was added LiAIH₄ (27 mL, 27.2 mmol, 1 M in THF) drop-wisely under N₂ atmosphere and the reaction mixture was stirred at 0° C. for 2 hours. The mixture was quenched with water (1 mL), 15% aq.NaOH solution (1 mL) and water (3 mL) successively at 0° C. The mixture was filtered and the filtrate was concentrated to dryness to give tert-butyl (S)-2-(hydroxymethyl)-4-methylenepyrrolidine-1-carboxylate (2.62 g, yield 63.9%) as light oil. LC/MS (ESI) m/z: 214 (M+H)⁺.

### Step 3: tert-butyl (S)-2-(((tert-butyldimethylsilyl)oxy)methyl)-4-methylenepyrrolidine-1-carboxylate (4)

To a solution of tert-butyl (S)-2-(hydroxymethyl)-4-methylenepyrrolidine-1-carboxylate (2.62 g, 12.3 mmol) in DMF (30 mL) was added imidazole (2.51 g, 36.9 mmol) and TBSCl (2.78 g, 18.5 mmol) at 0° C. under N₂ atmosphere and the mixture was stirred at 25° C. for 16 hours. The mixture was diluted with EtOAc, washed with water and brine, dried over anhydrous Na₂SO₄, filtered, and concentrated under reduced pressure to dryness. The residue was purified by column chromatography on silica gel (PE:EtOAc=10:1) to give tert-butyl (S)-2-(((tert-butyldimethylsilyl)oxy)methyl)-4-methylenepyrrolidine-1-carboxylate (4.0 g, yield 99.9%) as colorless oil. LC/MS (ESI) m/z: 328 (M+H)⁺.

### Step 4: tert-butyl (6S)-6-(((tert-butyldimethylsilyl)oxy)methyl)-1-oxa-5-azaspiro[2.4]heptane-5-carboxylate (5)

To a solution of tert-butyl (S)-2-(((tert-butyldimethylsilyl)oxy)methyl)-4-methylenepyrrolidine-1-carboxylate (4.91 g, 15.0 mmol) in DCM (40 mL) was added m-CPBA (5.16 g, 30.0 mmol) at 0° C. and the mixture was stirred at 25° C. for 16 hours. The mixture was quenched with saturated aq.Na₂S₂O₃ solution and extracted with DCM twice. The combined organic layers were washed with saturated aq.NaHCO₃ solution and brine, dried over anhydrous Na₂SO₄, filtered and concentrated under reduced pressure to dryness. The residue was purified by column chromatography on silica gel (PE:EtOAc=10:1) to give tert-butyl (6S)-6-(((tert-butyldimethylsilyl)oxy)methyl)-1-oxa-5-azaspiro[2.4]heptane-5-carboxylate (3.76 g, yield 73.0%) as colorless oil. LC/MS (ESI) m/z: 344 (M+H)⁺.

### Step 5: tert-butyl (7S)-7-(((tert-butyldimethylsilyl)oxy)methyl)-1-oxa-6-azaspiro[3.4]octane-6-carboxylate (6)

At 0° C., to a solution of trimethylsulfoxonium iodide (4.82 g, 21.92 mmol) in t-BuOH (20 mL) was added a solution of t-BuOK (2.46 g, 21.92 mmol) in t-BuOH (20 mL) and the mixture stirred at 50° C. for 0.5 hour. Then a solution of tert-butyl (6S)-6-(((tert-butyldimethylsilyl)oxy)methyl)-1-oxa-5-azaspiro[2.4]heptane-5-carboxylate (3.76 g, 10.96 mmol) in t-BuOH (20 mL) was added and the resulting mixture was stirred at 85° C. for 16 hours. The reaction mixture was quenched with ice water and extracted with DCM twice. The combined organic layers were washed with brine, dried over anhydrous Na₂SO₄ and concentrated to dryness under reduced pressure. The residue was purified by chromatography on silica gel (PE:EtOAc=10:1) to give tert-butyl (7S)-7-(((tert-butyldimethylsilyl)oxy)methyl)-1-oxa-6-azaspiro[3.4]octane-6-carboxylate (2.46 g, yield 63.1%) as light yellow oil. LC/MS (ESI) m/z: 358 (M+H)⁺.

### Step 6: tert-butyl (7S)-7-(hydroxymethyl)-1-oxa-6-azaspiro[3.4]octane-6-carboxylate (7)

To a solution of tert-butyl (7S)-7-(((tert-butyldimethylsilyl)oxy)methyl)-1-oxa-6-azaspiro[3.4]octane-6-carboxylate (2.46 g, 6.89 mmol) in THE (5 mL) was added TBAF (13.78 mL, 13.78 mmol, 1 M in THF) at 0° C. and the mixture was stirred at room temperature for 1 hour. The mixture was diluted with EtOAc (10 mL), washed with saturated aq.NH₄Cl solution and brine, dried over anhydrous Na₂SO₄, filtered and concentrated under reduced pressure to dryness to give tert-butyl (7S)-7-(hydroxymethyl)-1-oxa-6-azaspiro[3.4]octane-6-carboxylate (500 mg, yield 30.0%) as yellow oil, which was directly used in the next reaction. LC/MS (ESI) (m/z): 244 (M+H)⁺.

### Step 7: (7S)-6-(tert-butoxycarbonyl)-1-oxa-6-azaspiro[3.4]octane-7-carboxylic acid (8)

To a mixture of NaIO₄ (1.19 g, 5.56 mmol) and RuCl₃ (77 mg, 0.37 mmol) in water (5 mL) was added a solution of tert-butyl (7S)-7-(hydroxymethyl)-1-oxa-6-azaspiro[3.4]octane-6-carboxylate (450 mg, 1.85 mmol) in acetone (5 mL) and the mixture was stirred at 25° C. for 16 hours. The mixture was acidified with 1 M aq.H₂SO₄ to pH-4 and extracted with CHCl₃/i-PrOH (3/1, v/v) five times. The combined organic layers were washed with brine, dried over anhydrous Na₂SO₄, filtered and concentrated to give (7S)-6-(tert-butoxycarbonyl)-1-oxa-6-azaspiro[3.4]octane-7-carboxylic acid (330 mg, yield 69.3%) as brown oil, which was used directly in the next reaction. LC/MS (ESI) (m/z): 258 (M+H)⁺.

### Step 8: 7-benzyl 6-(tert-butyl) (7S)-1-oxa-6-azaspiro[3.4]octane-6,7-dicarboxylate (9)

To a solution of (7S)-6-(tert-butoxycarbonyl)-1-oxa-6-azaspiro[3.4]octane-7-carboxylic acid (150 mg, 0.58 mmol) in DMF (5 mL) was added K₂CO₃ (242 mg, 1.75 mmol) and benzyl bromide (150 mg, 0.87 mmol) and the reaction mixture was stirred at room temperature overnight. The mixture was diluted with EtOAc, washed with water and brine, dried over anhydrous Na₂SO₄, filtered and concentrated under reduced pressure to dryness to give 7-benzyl 6-(tert-butyl) (7S)-1-oxa-6-azaspiro[3.4]octane-6,7-dicarboxylate (200 mg, yield 99%) as yellow solid, which was used directly in the next reaction.

LC/MS (ESI) m/z: 348 (M+H)⁺.

### Step 9: benzyl (7S)-1-oxa-6-azaspiro[3.4]octane-7-carboxylate TFA salt (10)

To a solution of 7-benzyl 6-(tert-butyl) (7S)-1-oxa-6-azaspiro[3.4]octane-6,7-dicarboxylate (200 mg, 0.58 mmol) in DCM (2 mL) was added TFA (1 mL) and the mixture was stirred at room temperature for 1 hour. The reaction mixture was concentrated to dryness under reduced pressure, co-evaporated with DCM and dried under vacuum to give benzyl (7S)-1-oxa-6-azaspiro[3.4]octane-7-carboxylate TFA salt (190 mg, yield 100%) as brown oil, which was used directly in the next reaction without further purification. LC/MS (ESI) m/z: 248 (M+H)⁺.

### Step 10: benzyl (7S)-6-((4-phenoxybenzoyl)glycyl)-1-oxa-6-azaspiro[3.4]octane-7-carboxylate (11)

To a mixture of benzyl (7S)-1-oxa-6-azaspiro[3.4]octane-7-carboxylate TFA salt (190 mg, 0.58 mmol) and (4-phenoxybenzoyl)glycine (157 mg, 0.58 mmol) in DMF (3 mL) was added DIPEA (0.6 mL, 3.05 mmol) and T₃P (1.2 g, 1.83 mmol, 50% wt. in EtOAc) at 0° C. under N₂ atmosphere and the mixture was stirred at 25° C. for 2 hours. The mixture was diluted with EtOAc, washed with water and brine, dried over anhydrous Na₂SO₄, filtered and concentrated under reduced pressure to dryness. The residue was purified by flash chromatography (DCM:MeOH=20:1) to give benzyl (7S)-6-((4-phenoxybenzoyl)glycyl)-1-oxa-6-azaspiro[3.4]octane-7-carboxylate (140 mg, yield 46.2%) as yellow solid. LC/MS (ESI) m/z: 501 (M+H)⁺.

### Step 11: (7S)-6-((4-phenoxybenzoyl)glycyl)-1-oxa-6-azaspiro[3.4]octane-7-carboxylic acid (12)

To a solution of benzyl (7S)-6-((4-phenoxybenzoyl)glycyl)-1-oxa-6-azaspiro[3.4]octane-7-carboxylate (140 mg, 0.28 mmol) in MeOH (3 mL) was added Pd/C (20 mg, 10% wt), the mixture was degassed under N₂ atmosphere for three times and stirred under a H₂ balloon at room temperature overnight. The mixture was filtered and the filtrate was concentrated under reduce pressure to give (7S)-6-((4-phenoxybenzoyl)glycyl)-1-oxa-6-azaspiro[3.4]octane-7-carboxylic acid (110 mg, yield 96.5%) as yellow oil, which was used directly in the next reaction without further purification. LC/MS (ESI) (m/z): 411 (M+H)⁺.

### Step 12: (4R,7S)-N-((4-carbamimidoylthiophen-2-yl)methyl)-6-((4-phenoxybenzoyl)glycyl)-1-oxa-6-azaspiro[3.4]octane-7-carboxamide (Compound 194) and (4S,7S)-N-((4-carbamimidoylthiophen-2-yl)methyl)-6-((4-phenoxybenzoyl)glycyl)-1-oxa-6-azaspiro[3.4]octane-7-carboxamide (Compound 195)

To a mixture of (7S)-6-((4-phenoxybenzoyl)glycyl)-1-oxa-6-azaspiro[3.4]octane-7-carboxylic acid (55 mg, 0.13 mmol) and 5-(aminomethyl)thiophene-3-carboximidamide (42 mg, 0.26 mmol) in DMF (3 mL) was added DIPEA (0.11 mL, 0.45 mmol) and T₃P (102 mg, 0.16 mmol, 50% wt. in EtOAc) at 0° C. under N₂ atmosphere and the mixture was stirred at 25° C. for 3 hours. The mixture was quenched with water and extracted with CHCl₃/i-PrOH (3/1, v/v) twice. The combined organic layers were washed with brine, dried over anhydrous Na₂SO₄, filtered and concentrated under reduced pressure to dryness. The residue was purified by prep-TLC (DCM:MeOH=10:1) and further purified by prep-HPLC to give Compound 194 (5 mg, yield 6.8%) and Compound 195 (1.1 mg, yield 1.5%) as white solid. Compound 194: ¹H NMR (400 MHz, CD₃OD) δ 8.20 (d, J=1.6 Hz, 1H), 7.87-7.80 (m, 2H), 7.42-7.39 (m, 2H), 7.26 (s, 1H), 7.24 (s, 1H), 7.20 (s, 1H), 7.18 (s, 1H), 7.07 (s, 1H), 7.05 (s, 1H), 7.00 (d, J=4.8 Hz, 1H), 4.59-4.45 (m, 5H), 4.21 (s, 2H), 4.08 (t, J=8.8 Hz, 1H), 3.83 (d, J=11.4 Hz, 1H), 2.86-2.78 (m, 1H), 2.76-2.63 (m, 2H), 2.14 (dd, J=13.5, 8.3 Hz, 1H). LC/MS (ESI) (m/z): 548 (M+H)⁺; Compound 195 ¹H NMR (400 MHz, CD₃OD) δ 8.20 (d, J=1.6 Hz, 1H), 7.87-7.80 (m, 2H), 7.42-7.39 (m, 2H), 7.26 (s, 1H), 7.24 (s, 1H), 7.20 (s, 1H), 7.18 (s, 1H), 7.07 (s, 1H), 7.05 (s, 1H), 7.00 (d, J=4.8 Hz, 1H), 4.59-4.45 (m, 5H), 4.21 (s, 2H), 4.08 (t, J=8.8 Hz, 1H), 3.83 (d, J=11.4 Hz, 1H), 2.86-2.78 (m, 1H), 2.76-2.63 (m, 2H), 2.14 (dd, J=13.5, 8.3 Hz, 1H). LC/MS (ESI) (m/z): 548 (M+H)⁺.

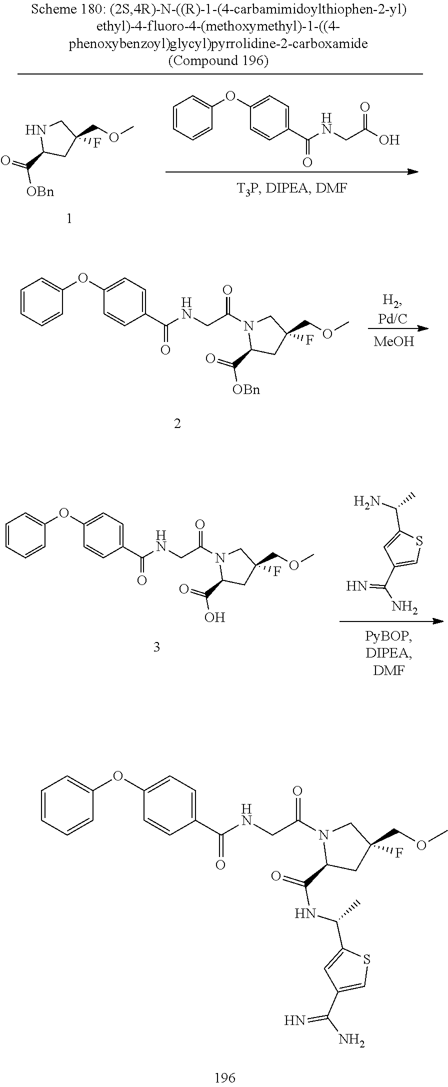

### Step 1: benzyl (2S,4R)-4-fluoro-4-(methoxymethyl)-1-((4-phenoxybenzoyl)glycyl) pyrrolidine-2-carboxylate (2)

At 0° C., to a mixture of benzyl (2S,4R)-4-fluoro-4-(methoxymethyl)pyrrolidine-2-carboxylate (50 mg, 0.19 mmol) and (4-phenoxybenzoyl)glycine (57 mg, 0.21 mmol) in DMF (2 mL) was added T₃P (363 mg, 0.57 mmol, 50% wt. in EtOAc) and DIPEA (0.2 mL, 1.14 mmol) under N₂ atmosphere and the mixture was stirred at 35° C. for 2 hours. The mixture was diluted with EtOAc and washed with saturated aq.NaHCO₃ solution. The aqueous layer was extracted with EtOAc twice and the combined organic layers were washed with brine, dried over anhydrous Na₂SO₄, filtered and concentrated under reduced pressure to dryness. The residue was purified by flash chromatography on silica gel (DCM:MeOH=20:1) to afford benzyl (2S,4R)-4-fluoro-4-(methoxymethyl)-1-((4-phenoxybenzoyl)glycyl)pyrrolidine-2-carboxylate (94 mg, yield 95.9%) as yellow solid. LC/MS (ESI) m/z: 521 (M+H)⁺.

### Step 2: (2S,4R)-4-fluoro-4-(methoxymethyl)-1-((4-phenoxybenzoyl)glycyl)pyrrolidine-2-carboxylic acid (3)

To a solution of benzyl (2S,4R)-4-fluoro-4-(methoxymethyl)-1-((4-phenoxybenzoyl)glycyl)pyrrolidine-2-carboxylate (94 mg, 0.18 mmol) in MeOH (2 mL) were added Pd/C (10 mg, 10% wt.) under N₂ atmosphere, the mixture was degassed under N₂ atmosphere for three times and stirred under a H₂ balloon at room temperature for 4 hours. The mixture was filtered and the filtrate was concentrated under reduced pressure to dryness to afford (2S,4R)-4-fluoro-4-(methoxymethyl)-1-((4-phenoxybenzoyl)glycyl)pyrrolidine-2-carboxylic acid (64 mg, yield 83.1%) as yellow oil, which was used directly in the next reaction. LC/MS (ESI) m/z: 431 (M+H)⁺.

### Step 3: (2S,4R)-N-((R)-1-(4-carbamimidoylthiophen-2-yl)ethyl)-4-fluoro-4-(methoxymethyl)-1-((4-phenoxybenzoyl)glycyl)pyrrolidine-2-carboxamide (Compound 196)

At 0° C., to a mixture of (2S,4R)-4-fluoro-4-(methoxymethyl)-1-((4-phenoxybenzoyl)glycyl)pyrrolidine-2-carboxylic acid (44 mg, 0.10 mmol) and (R)-5-(1-aminoethyl)thiophene-3-carboximidamide (20 mg, 0.12 mmol) in DMF (2 mL) was added PyBOP (78 mg, 0.15 mmol) and DIPEA (0.1 mL, 0.50 mmol) under N₂ atmosphere and the mixture was stirred at room temperature for 0.5 hour. The mixture was diluted with EtOAc, washed with water and brine, dried over anhydrous Na₂SO₄, filtered and concentrated under reduced pressure to dryness. The residue was purified by prep-HPLC to afford Compound 196 (3.8 mg, yield 6.6%) as a white solid. ¹H NMR (400 MHz, CD₃OD) δ 8.22 (d, J=9.6 Hz, 1H), 7.84 (dd, J=8.8, 15.2 Hz, 2H), 7.55 (d, J=7.6 Hz, 1H), 7.41 (t, J=7.2 Hz, 2H), 7.20 (t, J=7.0 Hz, 1H), 7.06 (d, J=8.4 Hz, 2H), 7.00 (d, J=47 Hz, 2H), 5.35-5.21 (m, 1H), 4.60 (t, J=8.6 Hz, 1H), 4.28 (d, J=16.8 Hz, 1H), 4.13-3.99 (m, 2H), 3.88 (dd, J=33.2, 34.0 Hz, 1H), 3.69 (dd, J=16.4, 18.0 Hz, 2H), 3.42 (d, J=4.8 Hz, 3H), 2.59-2.43 (m, 1H), 2.26-2.08 (m, 1H), 1.57 (d, J=6.9 Hz, 3H). LC/MS (ESI) m/z: 596 (M+H)⁺.

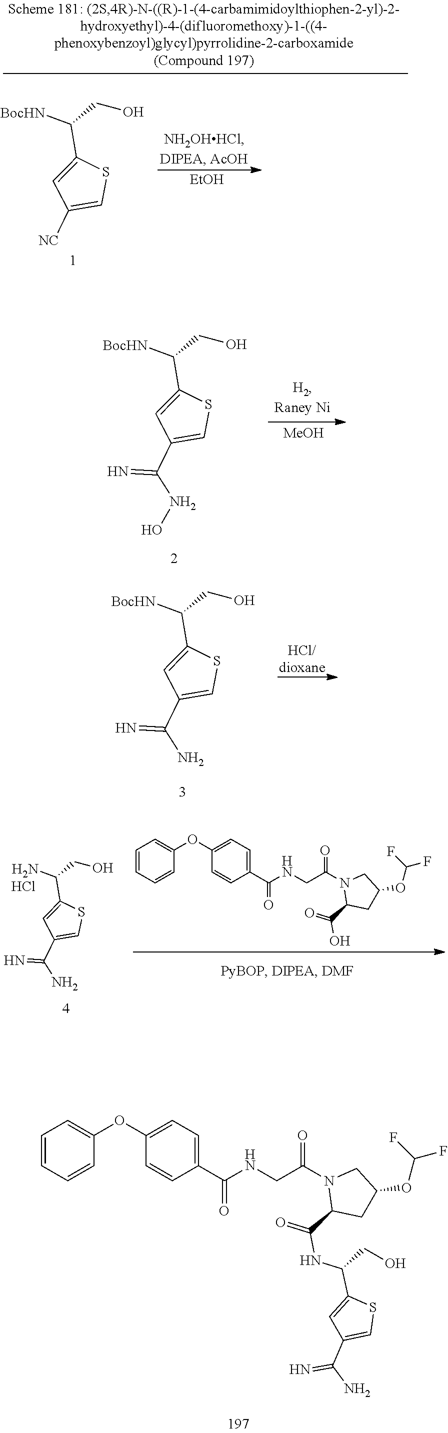

### Step 1: tert-butyl (R)-(2-hydroxy-1-(4-(N-hydroxycarbamimidoyl)thiophen-2-yl)ethyl) carbamate (2)

At 0° C., to a solution of tert-butyl (R)-(1-(4-cyanothiophen-2-yl)-2-hydroxyethyl)carbamate (400 mg, 1.49 mmol) in EtOH (5 mL) was added DIPEA (0.7 mL, 4.47 mmol) and NH₂OH·HCl (259 mg, 3.73 mmol) under N₂ atmosphere and the mixture was stirred at 30° C. for 5 hours. The mixture was dilute with EtOAc, washed with water and brine, dried over anhydrous Na₂SO₄, filtered and concentrated under reduced pressure to dryness. The residue was purified by flash chromatography on silica gel (DCM:MeOH=10:1) to afford tert-butyl (R)-(2-hydroxy-1-(4-(N-hydroxycarbamimidoyl)thiophen-2-yl)ethyl)carbamate (440 mg, yield 98.2%) as yellow oil. LC/MS (ESI) m/z: 302 (M+H)⁺.

### Step 2: tert-butyl (R)-(1-(4-carbamimidoylthiophen-2-yl)-2-hydroxyethyl)carbamate (3)

To a solution of tert-butyl (R)-(2-hydroxy-1-(4-(N-hydroxycarbamimidoyl)thiophen-2-yl)ethyl)carbamate (440 mg, 1.46 mmol) in MeOH (5 mL) were added Raney Ni (20 mg) and AcOH (one drop) under N₂ atmosphere, the mixture was degassed under N₂ atmosphere for three times and stirred under a H₂ balloon at 30° C. for 16 hours. The mixture was filtered and the filtrate was concentrated under reduced pressure to dryness to afford tert-butyl (R)-(1-(4-carbamimidoylthiophen-2-yl)-2-hydroxyethyl)carbamate (410 mg, yield 98.5%) as yellow oil, which was used directly in the next reaction. LC/MS (ESI) m/z: 286 (M+H)⁺.

### Step 3: (R)-5-(1-amino-2-hydroxyethyl)thiophene-3-carboximidamide hydrochloride (4)

At 0° C., to a solution of tert-butyl (R)-(1-(4-carbamimidoylthiophen-2-yl)-2-hydroxyethyl)carbamate (410 mg, 1.44 mmol) in HCl/1,4-dioxane (4 mL), and the mixture was stirred at 25° C. for 2 hours. The reaction mixture was concentrated to dryness, co-evaporated with DCM and dried under vacuum to afford (R)-5-(1-amino-2-hydroxyethyl)thiophene-3-carboximidamide hydrochloride (320 mg, yield 100%) as white solid, which was used directly in the next reaction. LC/MS (ESI) m/z: 186 (M+H)⁺.

### Step 4: (2S,4R)-N-((R)-1-(4-carbamimidoylthiophen-2-yl)-2-hydroxyethyl)-4-(difluoromethoxy)-1-((4-phenoxybenzoyl)glycyl)pyrrolidine-2-carboxamide (Compound 197)

At 0° C., to a mixture of (R)-5-(1-amino-2-hydroxyethyl)thiophene-3-carboximidamide hydrochloride (60 mg, 0.27 mmol) and (2S,4R)-4-(difluoromethoxy)-1-((4-phenoxybenzoyl) glycyl)pyrrolidine-2-carboxylic acid (117 mg, 0.27 mmol) in DMF (2 mL) was added PyBOP (211 mg, 0.41 mmol) and DIPEA (0.2 mL, 1.35 mmol) under N₂ atmosphere and the mixture was stirred at room temperature for 1 hour. The mixture was diluted with EtOAc, washed with water and brine, dried over anhydrous Na₂SO₄, filtered and concentrated under reduced pressure to dryness. The residue was purified by prep-HPLC to afford Compound 197 (19 mg, yield 11.7%) as a white solid. ¹H NMR (400 MHz, CD₃OD) δ 8.27-8.18 (m, 1H), 7.87-7.82 (m, 2H), 7.60-7.54 (m, 1H), 7.44-7.39 (m, 2H), 7.22-7.18 (m, 1H), 7.08-7.04 (m, 2H), 7.02-6.98 (m, 2H), 6.70-6.27 (m, 1H), 5.34-5.19 (m, 1H), 5.08-4.97 (m, 1H), 4.75-4.61 (m, 1H), 4.28-4.10 (m, 2H), 3.99-3.93 (m, 1H), 3.87 (d, J=5.8 Hz, 1H), 3.85-3.71 (m, 2H), 2.54-2.44 (m, 1H), 2.35-2.19 (m, 1H). LC/MS (ESI) m/z: 602 (M+H)⁺.

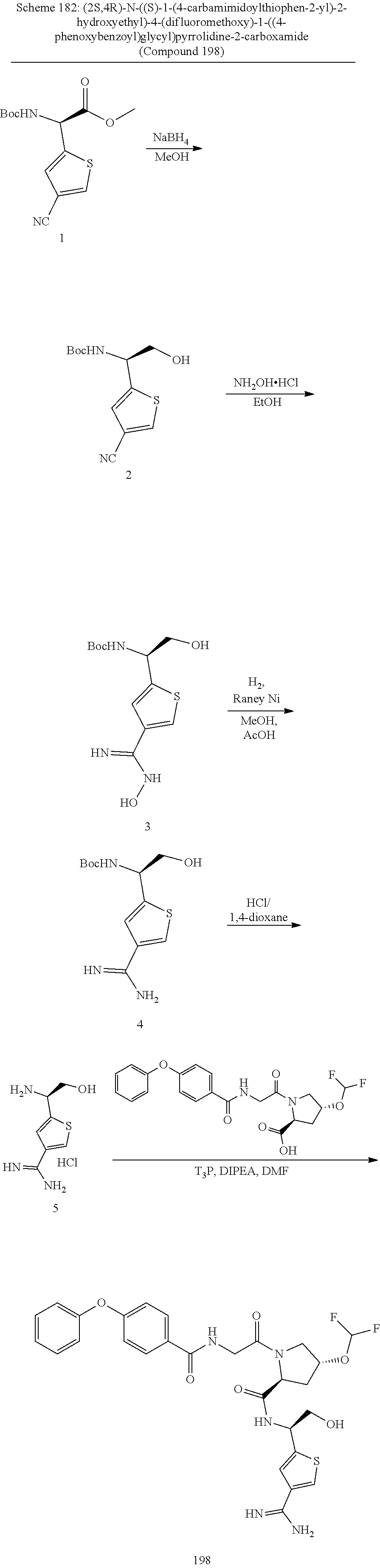

### Step 1: tert-butyl (S)-(1-(4-cyanothiophen-2-yl)-2-hydroxyethyl)carbamate (2)

At 0° C., to a solution of methyl 2-((tert-butoxycarbonyl)amino)-2-(4-cyanothiophen-2-yl)acetate (3.5 g, 11.82 mmol) in MeOH (50 mL) was added NaBH₄ (0.95 g, 23.64 mmol) in portions under N₂ atmosphere and the reaction mixture was stirred at 25° C. for 1 hour. The mixture was quenched with saturated aq.NH₄Cl solution and extracted with EtOAc twice. The combined organic layers were washed with brine, dried over anhydrous Na₂SO₄, filtered and concentrated under reduced pressure to dryness. The residue was purified by flash chromatography on silica gel (petroleum ether:EtOAc=1:1) and further purified by SFC to afford tert-butyl (S)-(1-(4-cyanothiophen-2-yl)-2-hydroxyethyl)carbamate (1.2 g, yield 37.5%) as colorless oil. LC/MS (ESI) m/z: 269 (M+H)⁺.

### Step 2: tert-butyl (S)-(2-hydroxy-1-(4-(N-hydroxycarbamimidoyl)thiophen-2-yl)ethyl)carbamate (3)

To a mixture of tert-butyl (S)-(1-(4-cyanothiophen-2-yl)-2-hydroxyethyl)carbamate (200 mg, 0.75 mmol) in EtOH (5 mL) was added DIPEA (0.6 mL, 3.75 mmol) and NH₂OH HCl (129 mg, 1.88 mmol) and the mixture was stirred at 30° C. for 4 hours. The mixture was diluted with EtOAc, washed with water and brine, dried over anhydrous Na₂SO₄, filtered and concentrated to dryness. The residue was purified by chromatography on silica gel (DCM:MeOH=10:1) to give tert-butyl (S)-(2-hydroxy-1-(4-(N-hydroxycarbamimidoyl)thiophen-2-yl)ethyl)carbamate (220 mg, yield 98.2%) as colorless oil. LC/MS (ESI) m/z: 302 (M+H)⁺.

### Step 3: tert-butyl (S)-(1-(4-carbamimidoylthiophen-2-yl)-2-hydroxyethyl)carbamate (4)

To a solution of tert-butyl (S)-(2-hydroxy-1-(4-(N-hydroxycarbamimidoyl)thiophen-2-yl)ethyl)carbamate (220 mg, 0.74 mmol) in MeOH (5 mL) and AcOH (0.02 mL) was added Raney Ni (20 mg), the mixture was degassed under N₂ atmosphere for three times and stirred under a H₂ balloon at room temperature overnight. The mixture was filtered and the filtrate was concentrated under reduce pressure to give tert-butyl (S)-(1-(4-carbamimidoylthiophen-2-yl)-2-hydroxyethyl)carbamate (200 mg, yield 96.2%) as colorless oil, which was used directly in the next reaction without further purification. LC/MS (ESI) (m/z): 286 (M+H)⁺.

### Step 4: (S)-5-(1-amino-2-hydroxyethyl)thiophene-3-carboximidamide hydrochloride (6)

A mixture of tert-butyl (S)-(1-(4-carbamimidoylthiophen-2-yl)-2-hydroxyethyl)carbamate (200 mg, 0.70 mmol) in HCl/1,4-dioxane (3 mL, 4M) was stirred at room temperature for 1 hour. The reaction mixture was concentrated to dryness under reduced pressure, co-evaporated with DCM and dried under vacuum to give (S)-5-(1-amino-2-hydroxyethyl)thiophene-3-carboximidamide hydrochloride (160 mg, yield 100%) as light yellow solid, which was used directly in the next reaction without further purification. LC/MS (ESI) m/z: 186 (M+H)⁺.

### Step 5: (2S,4R)-N-((S)-1-(4-carbamimidoylthiophen-2-yl)-2-hydroxyethyl)-4-(difluoromethoxy)-1-((4-phenoxybenzoyl)glycyl)pyrrolidine-2-carboxamide (Compound 198)

To a mixture of (S)-5-(1-amino-2-hydroxyethyl)thiophene-3-carboximidamide hydrochloride (46 mg, 0.21 mmol) and (2S,4R)-4-(difluoromethoxy)-1-((4-phenoxybenzoyl)glycyl)pyrrolidine-2-carboxylic acid (60 mg, 0.14 mmol) in DMF (3 mL) was added DIPEA (0.11 mL, 0.70 mmol) and T₃P (106 mg, 0.17 mmol, 50% wt. in EtOAc) at 0° C. under N₂ atmosphere and the mixture was stirred at 25° C. for 3 hours. The mixture was quenched with saturated aq.NaHCO₃ solution and extracted with CHCl₃/i-PrOH (3/1, v/v) twice. The combined organic layers were washed with brine, dried over anhydrous Na₂SO₄, filtered and concentrated under reduced pressure to dryness. The residue was purified by prep-TLC (DCM:MeOH=10:1) and further purified by prep-HPLC to give Compound 198 (20 mg, yield 24.1%) as white solid. ¹HNMR (400 MHz, CD₃OD) δ 8.12 (d, J=16.3 Hz, 1H), 7.88-7.81 (m, 2H), 7.52-7.39 (m, 3H), 7.20 (t, J=7.4 Hz, 1H), 7.09-7.04 (m, 2H), 7.00 (dd, J=11.7, 4.8 Hz, 2H), 6.49 (td, J=74.4, 17.8 Hz, 1H), 5.28 (dt, J=38.8, 5.8 Hz, 1H), 5.00 (s, 1H), 4.62 (t, J=7.8 Hz, 1H), 4.25-4.14 (m, 2H), 4.00 (dd, J=11.4, 4.7 Hz, 1H), 3.94-3.84 (m, 3H), 2.62-2.43 (m, 1H), 2.34-2.22 (m, 1H). LC/MS (ESI) (m/z): 602 (M+H)⁺.

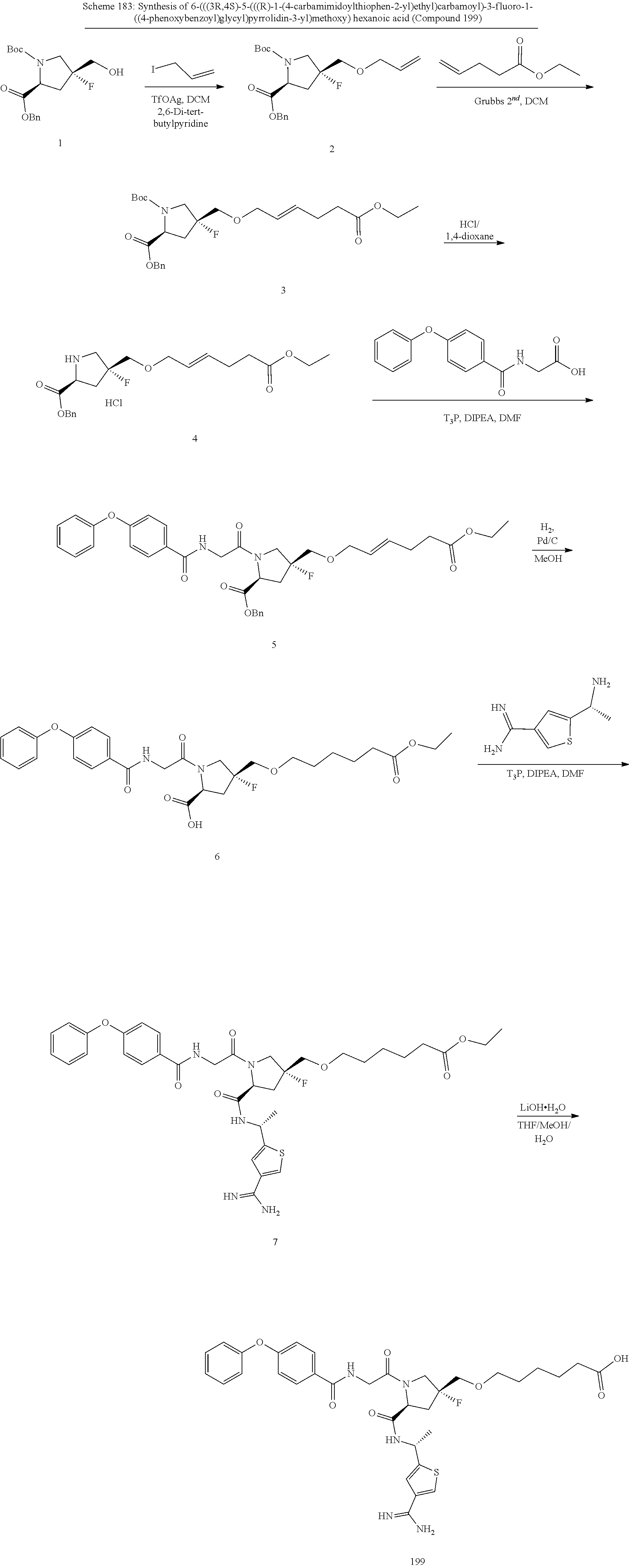

### Step 1: 2-benzyl 1-(tert-butyl) (2S,4R)-4-((allyloxy)methyl)-4-fluoropyrrolidine-1,2-dicarboxylate

To a solution of 2-benzyl 1-(tert-butyl) (2S,4R)-4-fluoro-4-(hydroxymethyl)pyrrolidine-1,2-dicarboxylate (300 mg, 0.85 mmol) in DCE (3 mL) was added AgOTf (655 mg, 2.55 mmol) followed by 3-iodoprop-1-ene (0.38 mL, 4.25 mmol) and 2,6-di-tert-butylpyridine (0.57 mL, 2.55 mmol) and the mixture was stirred at 30° C. for 16 hours under N₂ atmosphere. The mixture was filtered, and the filtrate was concentrated to dryness. The residue was purified by flash chromatography on silica gel (PE:EtOAc=5:1) to give 2-benzyl 1-(tert-butyl) (2S,4R)-4-((allyloxy)methyl)-4-fluoropyrrolidine-1,2-dicarboxylate (200 mg, yield 59.9%) as colorless oil. LC/MS (ESI) (m/z): 394 (M+H)⁺.

### Step 2: 2-benzyl 1-(tert-butyl) (2S,4R)-4-((((E)-6-ethoxy-6-oxohex-2-en-1-yl)oxy)methyl)-4-fluoropyrrolidine-1,2-dicarboxylate

To a solution of 2-benzyl 1-(tert-butyl) (2S,4R)-4-((allyloxy)methyl)-4-fluoropyrrolidine-1,2-dicarboxylate (200 mg, 0.51 mmol) in dry DCM (140 mL) was added Grubbs 2^{nd} catalyst (112 mg, 0.13 mmol) and ethyl pent-4-enoate (81 mg, 0.61 mmol) and the mixture was stirred at 30° C. under N₂ atmosphere for 8 hours. The mixture was concentrated to dryness under reduced pressure and the residue was purified by flash chromatography (PE:EtOAc=5:1) to give 2-benzyl 1-(tert-butyl) (2S,4R)-4-((((E)-6-ethoxy-6-oxohex-2-en-1-yl)oxy)methyl)-4-fluoropyrrolidine-1,2-dicarboxylate (45 mg, yield 18%) as white solid. LC/MS (ESI) m/z: 494 (M+H)⁺.

### Step 3: benzyl (2S,4R)-4-((((E)-6-ethoxy-6-oxohex-2-en-1-yl)oxy)methyl)-4-fluoropyrrolidine-2-carboxylate hydrochloride

A solution of 2-benzyl 1-(tert-butyl) (2S,4R)-4-((((E)-6-ethoxy-6-oxohex-2-en-1-yl)oxy)methyl)-4-fluoropyrrolidine-1,2-dicarboxylate (45 mg, 0.09 mmol) in HCl/1,4-dioxane (3 mL, 4M) was stirred at room temperature for 1 hour. The reaction mixture was concentrated to dryness under reduced pressure, co-evaporated with DCM and dried under vacuum to give benzyl (2S,4R)-4-((((E)-6-ethoxy-6-oxohex-2-en-1-yl)oxy)methyl)-4-fluoropyrrolidine-2-carboxylate hydrochloride (40 mg, yield 100%) as yellow solid, which was used directly in the next step without further purification. LC/MS (ESI) m/z: 394 (M+H)⁺.

### Step 4: benzyl (2S,4R)-4-((((E)-6-ethoxy-6-oxohex-2-en-1-yl)oxy)methyl)-4-fluoro-1-((4-phenoxybenzoyl)glycyl)pyrrolidine-2-carboxylate

To a mixture of benzyl (2S,4R)-4-((((E)-6-ethoxy-6-oxohex-2-en-1-yl)oxy)methyl)-4-fluoropyrrolidine-2-carboxylate (40 mg, 0.09 mmol) and (4-phenoxybenzoyl)glycine (28 mg, 0.10 mmol) in DMF (3.0 mL) was added DIPEA (0.08 mL, 0.50 mmol) and T₃P (197 mg, 0.30 mmol, 50% wt. in EtOAc) at 0° C. under N₂ atmosphere and the mixture was stirred at 25° C. for 2 hours. The mixture was diluted with EtOAc and washed with water and brine, dried over anhydrous Na₂SO₄, filtered and concentrated under reduced pressure to dryness. The residue was purified by flash chromatography on silica gel (PE:EtOAc=1:1) to give benzyl (2S,4R)-4-((((E)-6-ethoxy-6-oxohex-2-en-1-yl)oxy)methyl)-4-fluoro-1-((4-phenoxybenzoyl)glycyl) pyrrolidine-2-carboxylate (38 mg, yield 63.2%) as yellow solid. LC/MS (ESI) m/z: 647 (M+H)⁺.

### Step 5: (2S,4R)-4-(((6-ethoxy-6-oxohexyl)oxy)methyl)-4-fluoro-1-((4-phenoxybenzoyl)glycyl) pyrrolidine-2-carboxylic acid

To a solution of benzyl (2S,4R)-4-((((E)-6-ethoxy-6-oxohex-2-en-1-yl)oxy)methyl)-4-fluoro-1-((4-phenoxybenzoyl)glycyl)pyrrolidine-2-carboxylate (34 mg, 0.05 mmol) in MeOH (3 mL) was added Pd/C (5 mg, 10% wt) at 0° C., and the reaction mixture was stirred under a H₂ balloon at 25° C. for 2 hours. The mixture was filtered and the filtrate was concentrated to dryness under reduced pressure give (2S,4R)-4-(((6-ethoxy-6-oxohexyl)oxy)methyl)-4-fluoro-1-((4-phenoxybenzoyl)glycyl)pyrrolidine-2-carboxylic acid (32 mg, yield 100%) as colorless oil, which was used directly in the next step. LC/MS (ESI) m/z: 559 (M+H)⁺.

### Step 6: ethyl 6-(((3R,5S)-5-(((R)-1-(4-carbamimidoylthiophen-2-yl)ethyl)carbamoyl)-3-fluoro-1-((4-phenoxybenzoyl)glycyl)pyrrolidin-3-yl)methoxy)hexanoate

To a mixture of (2S,4R)-4-(((6-ethoxy-6-oxohexyl)oxy)methyl)-4-fluoro-1-((4-phenoxybenzoyl)glycyl)pyrrolidine-2-carboxylic acid (32 mg, 0.06 mmol) in DMF (3.0 mL) was added DIPEA (0.05 mL, 0.30 mmol) and T₃P (44 mg, 0.07 mmol, 50% wt. in EtOAc) under N₂ atmosphere and the mixture was stirred at 25° C. for 2 hours. The mixture was diluted with EtOAc, washed with water and brine, dried over anhydrous Na₂SO₄, filtered and concentrated under reduced pressure to dryness. The residue was purified by prep-TLC (DCM:MeOH=10:1) to give ethyl 6-(((3R,5S)-5-(((R)-1-(4-carbamimidoylthiophen-2-yl)ethyl)carbamoyl)-3-fluoro-1-((4-phenoxybenzoyl)glycyl)pyrrolidin-3-yl)methoxy)hexanoate (34 mg, yield 85.0%) as white solid. LC/MS (ESI) m/z: 710 (M+H)⁺.

### Step 7: 6-(((3R,5S)-5-(((R)-1-(4-carbamimidoylthiophen-2-yl)ethyl)carbamoyl)-3-fluoro-1-((4-phenoxybenzoyl)glycyl)pyrrolidin-3-yl)methoxy)hexanoic acid (Compound 199)

To a solution of ethyl 6-(((3R,5S)-5-(((R)-1-(4-carbamimidoylthiophen-2-yl)ethyl)carbamoyl)-3-fluoro-1-((4-phenoxybenzoyl)glycyl)pyrrolidin-3-yl)methoxy)hexanoate (34 mg, 0.05 mmol) in MeOH (2 mL) and THE (1 mL) and water (0.6 mL) was added lithium hydroxide (3.0 mg, 0.07 mmol) and the mixture was stirred at room temperature for 2 hours. The mixture was acidified with 1N aq.HCl to pH-3 and extracted with DCM three times. The combined organic layers were washed with brine, dried over anhydrous Na₂SO₄, filtered and concentrated to dryness. The residue was purified by prep-HPLC to give Compound 199 (1.1 mg, yield 3.3%) as a white solid. ¹H NMR (400 MHz, CD₃OD) δ 8.51 (s, 1H), 8.25 (t, J=2.8 Hz, 1H), 7.84 (t, J=6.7 Hz, 2H), 7.54 (d, J=9.6 Hz, 1H), 7.41 (t, J=7.9 Hz, 2H), 7.20 (t, J=7.4 Hz, 1H), 7.06 (d, J=8.4 Hz, 2H), 7.03-6.97 (m, 2H), 5.27 (d, J=7.0 Hz, 1H), 4.61 (s, 1H), 4.24 (d, J=16.6 Hz, 1H), 4.14 (d, J=16.6 Hz, 1H), 4.07-3.99 (m, 1H), 3.97-3.88 (m, 1H), 3.72 (d, J=7.0 Hz, 1H), 3.66 (d, J=10.7 Hz, 1H), 3.53 (t, J=6.3 Hz, 2H), 2.58-2.48 (m, 1H), 2.25 (dd, J=17.7, 10.6 Hz, 3H), 1.64 (d, J=5.1 Hz, 1H), 1.62 (s, 1H), 1.61-1.55 (m, 5H), 1.41 (dd, J=15.2, 8.4 Hz, 2H). LC/MS (ESI) m/z: 682 (M+H)⁺.

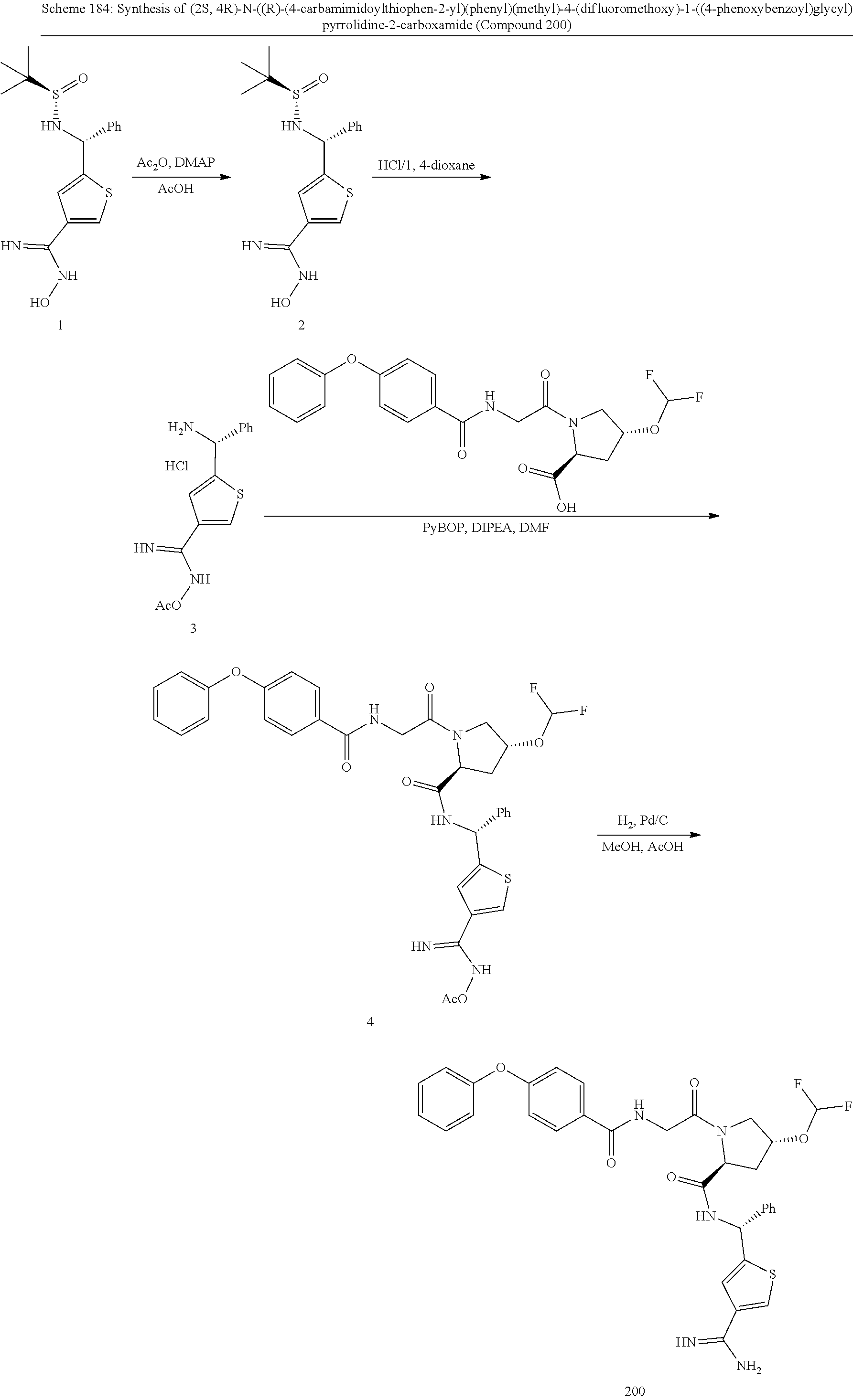

### Step 1: N-acetoxy-5-((1R)-((tert-butylsulfinyl)amino)(phenyl)methyl)thiophene-3-carboximidamide (2)

To a solution of 5-((1R)-((tert-butylsulfinyl)amino)(phenyl)methyl)-N-hydroxythiophene-3-carboximidamide (195 mg, 0.55 mmol) in AcOH (1 mL) was added DMAP (5 mg) and Ac₂O (1 mL) under N₂ atmosphere and the reaction mixture was stirred at room temperature overnight. The mixture was diluted with EtOAc and washed with water and brine, dried over anhydrous Na₂SO₄, filtered and concentrated under reduced pressure to dryness. The residue was purified by flash chromatography on silica gel (silica gel: 40-60% EtOAc in PE) to give N-acetoxy-5-((1R)-((tert-butylsulfinyl)amino)(phenyl)methyl)thiophene-3-carboximidamide (170 mg, yield 77.9%) as white solid. LC/MS (ESI) m/z:394 (M+H)⁺.

### Step 2: (R)-N-acetoxy-5-(amino(phenyl)methyl)thiophene-3-carboximidamide hydrochloride (3)

To a solution of N-acetoxy-5-((1R)-((tert-butylsulfinyl)amino)(phenyl)methyl)thiophene-3-carboximidamide (170 mg, 0.43 mmol) in 1,4-dioxane (2 mL) was added HCl/1,4-dioxane (1 mL) under N₂ atmosphere and the reaction mixture was stirred at room temperature for 2 hours. The mixture was concentrated under reduced pressure to dryness to give (R)-N-acetoxy-5-(amino(phenyl)methyl)thiophene-3-carboximidamide hydrochloride (140 mg, yield 100%) as white solid, which was directly used in the next reaction without purification. LC/MS (ESI) m/z:290 (M+H)⁺.

### Step 3: (2S,4R)-N-((R)-(4-(N-acetoxycarbamimidoyl)thiophen-2-yl)(phenyl)methyl)-4-(difluoromethoxy)-1-((4-phenoxybenzoyl)glycyl)pyrrolidine-2-carboxamide (4)

A solution of (R)-N-acetoxy-5-(amino(phenyl)methyl)thiophene-3-carboximidamide hydrochloride (68 mg, 0.21 mmol) and (2S,4R)-4-(difluoromethoxy)-1-((4-phenoxybenzoyl) glycyl)pyrrolidine-2-carboxylic acid (93 mg, 0.21 mmol) in DMF (5 mL) was added DIPEA (166 mg, 1.29 mmol) and PyBop (167 mg, 0.32 mmol) under N₂ atmosphere and the mixture was stirred at room temperature overnight. The mixture was diluted with EtOAc, washed with water and brine, dried over anhydrous Na₂SO₄, filtered and concentrated under reduced pressure to dryness. The residue was purified by flash chromatography on silica gel (PE:EtOAc=3:1) to give methyl (2S,4R)-4-(methylthio)-1-((4-phenoxybenzoyl)glycyl)pyrrolidine-2-carboxylate (150 mg, yield 99.2%) as white solid. LC/MS (ESI) m/z:706 (M+H)⁺.

### Step 4: (2S,4R)-N-((R)-(4-carbamimidoylthiophen-2-yl)(phenyl)methyl)-4-(difluoromethoxy)-1-((4-phenoxybenzoyl)glycyl)pyrrolidine-2-carboxamide (Compound 200)

To a solution of (2S,4R)-N-((R)-(4-(N-acetoxycarbamimidoyl)thiophen-2-yl)(phenyl)methyl)-4-(difluoromethoxy)-1-((4-phenoxybenzoyl)glycyl)pyrrolidine-2-carboxamide (75 mg, 0.11 mmol) in MeOH (3 mL) was added Pd/C (100 mg, 10% wt.) and AcOH (one drop) and the reaction mixture was stirred under a H₂ balloon at 30° C. overnight. The mixture was filtered and the filtrate was concentrated under reduced pressure to dryness. The residue was purified by prep-TLC (DCM:MeOH=5:1) and further purified by prep-HPLC to give Compound 200 (20 mg, yield 29.1%) as white solid. ¹HNMR (400 MHz, CD₃OD) δ 8.52 (s, 1H), 8.32-8.21 (m, 1H), 7.87-7.77 (m, 2H), 7.54-7.34 (m, 8H), 7.21 (t, J=7.4 Hz, 1H), 7.08-6.98 (m, 4H), 6.69-6.32 (m, 2H), 5.00 (s, 1H), 4.68 (t, J=7.8 Hz, 1H), 4.32-4.13 (m, 2H), 3.93 (dt, J=26.2, 7.5 Hz, 2H), 2.61-2.40 (m, 1H), 2.28-2.14 (m, 1H). LC/MS (ESI) m/z:648 (M+H)⁺.

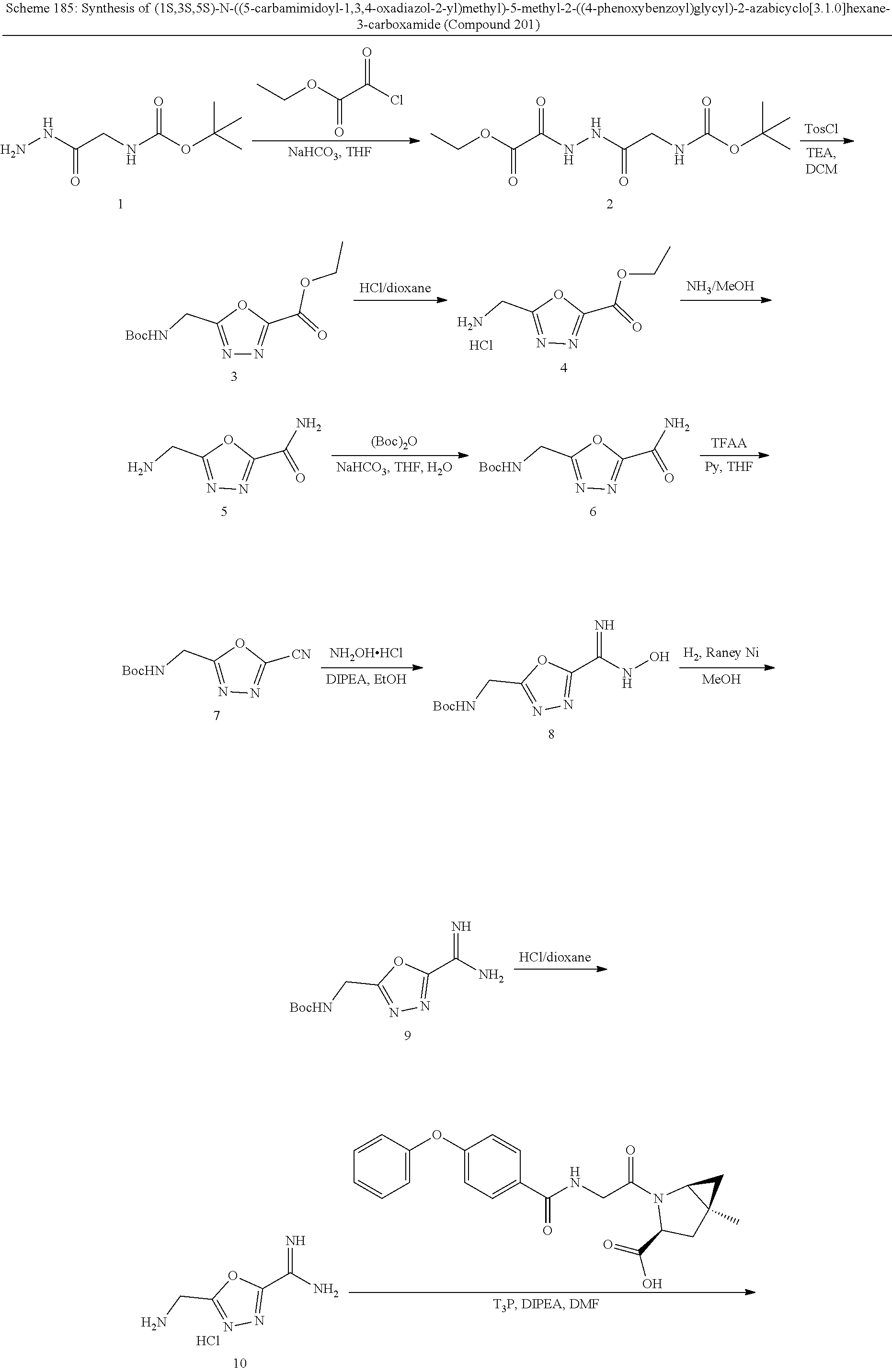

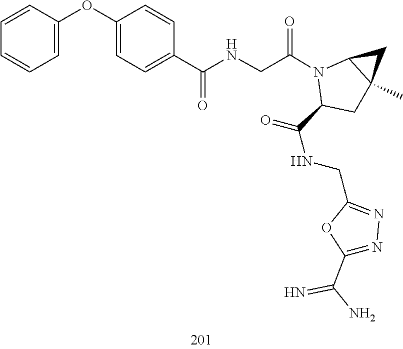 [SMILES: C[C@@]12C[C@@H]1N(C(=O)CNC(=O)c1ccc(Oc3ccccc3)cc1)[C@H](C(=O)NCc1nnc(C(=N)N)o1)C2]

### Step 1: ethyl 2-(2-((tert-butoxycarbonyl)glycyl)hydrazinyl)-2-oxoacetate

To a solution of tert-butyl (2-hydrazinyl-2-oxoethyl)carbamate (3.0 g, 15.87 mmol) in THE (20 mL) was added NaHCO₃ (2.66 g, 31.74 mmol) and a solution of ethyl 2-chloro-2-oxoacetate (2.16 g, 15.87 mmol) in THE (10 mL) at 0° C., and the mixture was stirred at 30° C. for 2 hours. The mixture was diluted with EtOAc, washed with water and brine, dried over anhydrous Na₂SO₄, filtered and concentrated to dryness. The residue was purified by chromatography on silica gel (DCM:MeOH=20:1) to give ethyl 2-(2-((tert-butoxycarbonyl)glycyl)hydrazinyl)-2-oxoacetate (2.5 g, yield 76.1%) as white solid. LC/MS (ESI) m/z: 290 (M+H)⁺.

### Step 2: ethyl 5-(((tert-butoxycarbonyl)amino)methyl)-1,3,4-oxadiazole-2-carboxylate

To a mixture of ethyl 2-(2-((tert-butoxycarbonyl)glycyl)hydrazinyl)-2-oxoacetate (2.16 g, 7.47 mmol) and TEA (3.12 mL, 22.41 mmol) in DCM (20 mL) was added a solution of tosyl chloride (2.14 g, 14.94 mmol) in DCM (10 mL) drop-wisely at 0° C. and the mixture was stirred at 30° C. for 16 hours. The mixture was diluted with DCM, washed with water and brine, dried over anhydrous Na₂SO₄, filtered and concentrated to dryness. The residue was purified by chromatography on silica gel (DCM:MeOH=20:1) to give ethyl 5-(((tert-butoxycarbonyl)amino)methyl)-1,3,4-oxadiazole-2-carboxylate (1.5 g, yield 75.0%) as yellow oil. LC/MS (ESI) m/z: 272 (M+H)⁺.

### Step 3: ethyl 5-(aminomethyl)-1,3,4-oxadiazole-2-carboxylate hydrochloride

A solution of ethyl 5-(((tert-butoxycarbonyl)amino)methyl)-1,3,4-oxadiazole-2-carboxylate (1.5 g, 5.54 mmol) in HCl/1,4-dioxane (10 mL, 4M) was stirred at room temperature for 1 hour. The reaction mixture was concentrated to dryness under reduced pressure, co-evaporated with DCM and dried under vacuum to give ethyl 5-(aminomethyl)-1,3,4-oxadiazole-2-carboxylate (1.2 g, yield 100%) as yellow solid, which was used directly in the next reaction without further purification. LC/MS (ESI) m/z: 172 (M+H)⁺.

### Step 4: 5-(aminomethyl)-1,3,4-oxadiazole-2-carboxamide

A solution of ethyl 5-(aminomethyl)-1,3,4-oxadiazole-2-carboxylate hydrochloride (1.2 g, 5.54 mmol) in NH₃/MeOH (10 mL, 7M) was stirred at room temperature for 16 hours. The reaction mixture was concentrated to dryness under reduced pressure, co-evaporated with DCM and dried under vacuum to give 5-(aminomethyl)-1,3,4-oxadiazole-2-carboxamide (786 mg, yield 100%) as yellow solid, which was used directly in the next reaction without further purification. LC/MS (ESI) m/z: 143 (M+H)⁺.

### Step 5: tert-butyl ((5-carbamoyl-1,3,4-oxadiazol-2-yl)methyl)carbamate

To a mixture of 5-(aminomethyl)-1,3,4-oxadiazole-2-carboxamide (789 mg, 5.54 mmol) and di-tert-butyl dicarbonate (1.8 g, 8.31 mmol) in THE (20 mL) was added a solution of NaHCO₃ (2.3 g, 27.7 mmol) in H₂O (10 mL) drop-wisely at 0° C. and the mixture was stirred at 25° C. for 16 hours. The mixture was diluted with EtOAc, washed with water and brine, dried over anhydrous Na₂SO₄, filtered and concentrated to dryness. The residue was purified by chromatography on silica gel (DCM:MeOH=20:1) to give tert-butyl ((5-carbamoyl-1,3,4-oxadiazol-2-yl)methyl)carbamate (600 mg, yield 44.6%) as yellow oil. LC/MS (ESI) m/z: 243 (M+H)⁺.

### Step 6: tert-butyl ((5-cyano-1,3,4-oxadiazol-2-yl)methyl)carbamate

To a mixture of tert-butyl ((5-carbamoyl-1,3,4-oxadiazol-2-yl)methyl)carbamate (300 mg, 1.24 mmol) and TFAA (781 mg, 3.72 mmol) in THE (5 mL) was added pyridine (1 mL, 12.4 mmol) drop-wisely at 0° C. and the mixture was stirred at 25° C. for 2 hours. The mixture was diluted with EtOAc, washed with water and brine, dried over anhydrous Na₂SO₄, filtered and concentrated under reduced pressure to dryness to give tert-butyl ((5-cyano-1,3,4-oxadiazol-2-yl)methyl)carbamate (277 mg, yield 100%) as brown oil, which was used directly in the next reaction without further purification. LC/MS (ESI) m/z: 225 (M+H)⁺.

### Step 7: tert-butyl ((5-(N-hydroxycarbamimidoyl)-1,3,4-oxadiazol-2-yl)methyl)carbamate

To a mixture of tert-butyl ((5-cyano-1,3,4-oxadiazol-2-yl)methyl)carbamate (340 mg, 1.5 mmol) in EtOH (5 mL) was added DIPEA (1.25 mL, 7.5 mmol) and NH₂OH·HCl (264 mg, 3.75 mmol) and the mixture was stirred at 30° C. for 4 hours. The mixture was diluted with EtOAc, washed with water and brine, dried over Na₂SO₄, filtered and concentrated to dryness. The residue was purified by chromatography on silica gel (DCM:MeOH=20:1) to give tert-butyl ((5-(N-hydroxycarbamimidoyl)-1,3,4-oxadiazol-2-yl)methyl)carbamate (160 mg, yield 41.0%) as yellow solid. LC/MS (ESI) m/z: 258 (M+H)⁺.

### Step 8: tert-butyl ((5-carbamimidoyl-1,3,4-oxadiazol-2-yl)methyl)carbamate

To a solution of tert-butyl ((5-(N-hydroxycarbamimidoyl)-1,3,4-oxadiazol-2-yl)methyl)carbamate (160 mg, 0.62 mmol) in MeOH (5 mL) and AcOH (0.02 mL) was added Raney Ni (20 mg), the mixture was degassed under N₂ atmosphere for three times and stirred under a H₂ balloon at room temperature overnight. The mixture was filtered and the filtrate was concentrated under reduce pressure to give tert-butyl ((5-carbamimidoyl-1,3,4-oxadiazol-2-yl)methyl)carbamate (120 mg, yield 80%) as colorless oil, which was used directly in the next reaction without further purification. LC/MS (ESI) (m/z): 242 (M+H)⁺.

### Step 9: 5-(aminomethyl)-1,3,4-oxadiazole-2-carboximidamide hydrochloride

A mixture of tert-butyl ((5-carbamimidoyl-1,3,4-oxadiazol-2-yl)methyl)carbamate (120 mg, 0.50 mmol) in HCl/1,4-dioxane (3 mL, 4M) was stirred at room temperature for 1 hour. The reaction mixture was concentrated to dryness under reduced pressure, co-evaporated with DCM and dried under vacuum to give 5-(aminomethyl)-1,3,4-oxadiazole-2-carboximidamide hydrochloride (90 mg, yield 100%) as light yellow solid, which was used directly in the next reaction without further purification. LC/MS (ESI) m/z: 142 (M+H)⁺.

### Step 10: (1S,3S,5S)-N-((5-carbamimidoyl-1,3,4-oxadiazol-2-yl)methyl)-5-methyl-2-((4-phenoxybenzoyl)glycyl)-2-azabicyclo[3.1.0]hexane-3-carboxamide (Compound 201)

To a mixture of 5-(aminomethyl)-1,3,4-oxadiazole-2-carboximidamide hydrochloride (71 mg, 0.40 mmol) and (1 S,3S,5S)-5-methyl-2-((4-phenoxybenzoyl)glycyl)-2-azabicyclo[3.1.0]hexane-3-carboxylic acid (78 mg, 0.20 mmol) in DMF (3 mL) was added DIPEA (0.16 mL, 1.0 mmol) and T₃P (151 mg, 0.24 mmol, 50% wt. in EtOAc) at 0° C. under N₂ atmosphere and the mixture was stirred at 25° C. for 3 hours. The mixture was quenched with saturated aq.NaHCO₃ solution and extracted with CHCl₃/i-PrOH (3/1, v/v) twice. The combined organic layers were washed with brine, dried over anhydrous Na₂SO₄, filtered and concentrated under reduced pressure to dryness. The residue was purified by prep-TLC (DCM:MeOH=10:1) and further purified by prep-HPLC to Compound 201 (5 mg, yield 5.0%) as a white solid. ¹H NMR (400 MHz, CD₃OD) δ 8.50 (s, 1H), 7.86-7.82 (m, 2H), 7.42 (dd, J=8.5, 7.5 Hz, 2H), 7.21 (t, J=7.4 Hz, 1H), 7.08-7.05 (m, 2H), 7.02-6.99 (m, 2H), 4.71 (s, 2H), 4.33 (d, J=7.3 Hz, 2H), 3.41 (dd, J=6.0, 2.4 Hz, 1H), 2.44 (t, J=11.8 Hz, 1H), 2.22 (dd, J=13.4, 3.5 Hz, 1H), 1.31 (s, 3H), 1.23 (dd, J=5.8, 2.5 Hz, 1H), 0.82 (t, J=5.3 Hz, 1H). LC/MS (ESI) (m/z): 518 (M+H)⁺.

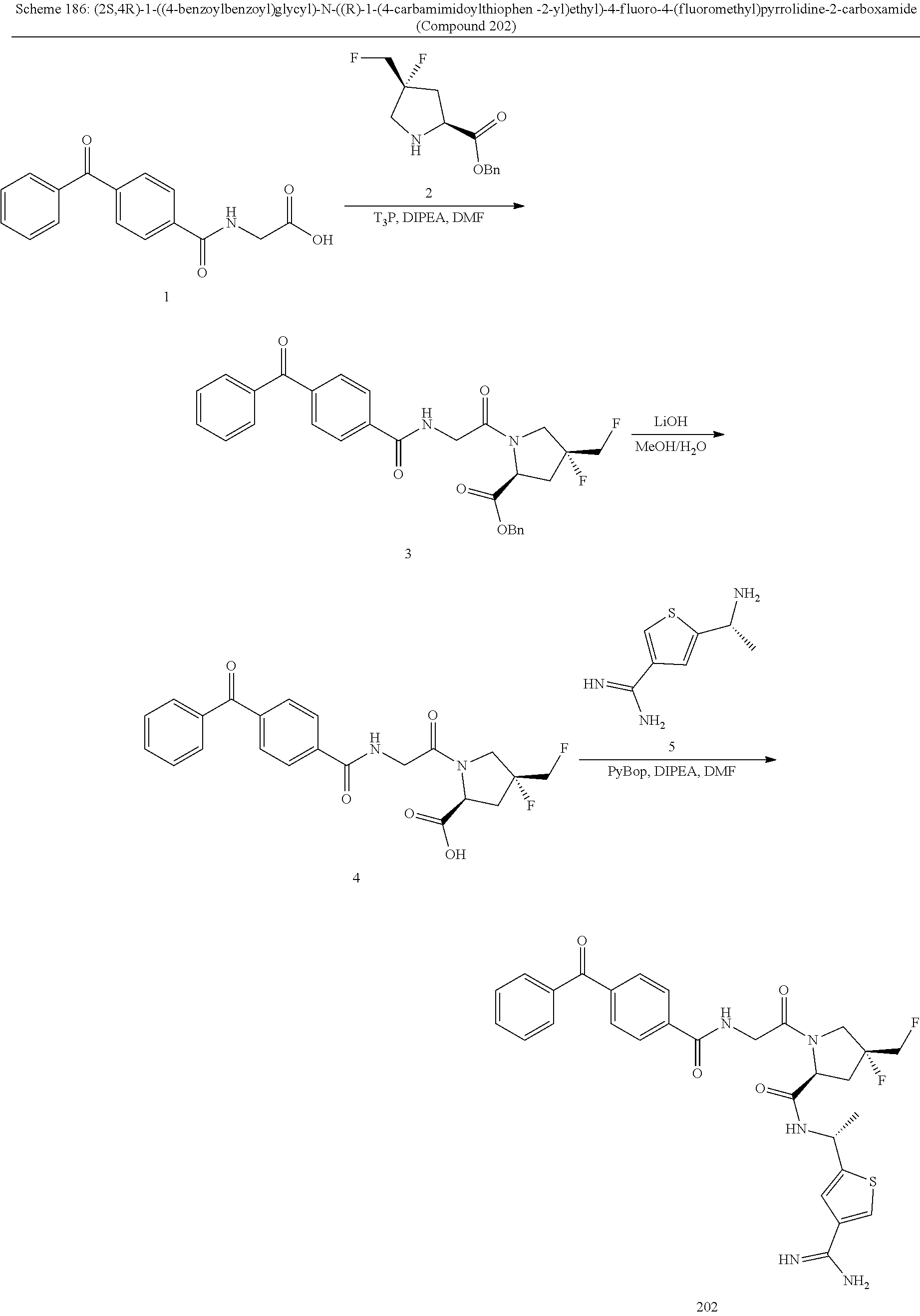

### Step 1: tert-butyl (2S,4R)-2-(((tert-butyldiphenylsilyl)oxy)methyl)-4-hydroxypyrrolidine-1-carboxylate

To a mixture of (4-benzoylbenzoyl)glycine (33 mg, 0.12 mol) and benzyl (4S)-4-(fluoromethyl)-4-methylpyrrolidine-2-carboxylate (30 mg, 0.12 mmol) in DMF (1.0 mL) was added DIPEA (91 mg, 0.71 mmol) and T₃P (56 mg, 0.18 mmol) at 0° C. and the mixture was stirred under N₂ atmosphere at room temperature for 1 hour. The mixture was quenched with saturated NaHCO₃ solution and extracted with EtOAc twice. The combined organic layers were washed with brine, dried over anhydrous Na₂SO₄, filtered and concentrated under reduced pressure to dryness. The residue was purified by flash chromatography on silica gel (PE:EtOAc=1:1) to give benzyl (2S,4R)-1-((4-benzoylbenzoyl)glycyl)-4-fluoro-4-(fluoromethyl)pyrrolidine-2-carboxylate (53 mg, yield 86.3%) as colorless oil. LC/MS (ESI) (m/z): 449 (M+H)⁺.

### Step 2: (2S,4R)-1-((4-benzoylbenzoyl)glycyl)-4-fluoro-4-(fluoromethyl)pyrrolidine-2-carboxylic acid

To a solution of benzyl (2S,4R)-1-((4-benzoylbenzoyl)glycyl)-4-fluoro-4-(fluoromethyl)pyrrolidine-2-carboxylate (53 mg, 0.60 mmol) in MeOH (1 mL) and water (1 mL) was added LiOH (25 mg, 0.61 mmol) at 0° C. and the mixture was stirred at room temperature for 2 hours. The mixture was acidified with 1N aq.HCl to pH=3 and extracted with EtOAc twice. The combined organic layers were washed with brine, dried over anhydrous Na₂SO₄, filtered and concentrated under reduced pressure to dryness to give (2S,4R)-1-((4-benzoylbenzoyl)glycyl)-4-fluoro-4-(fluoromethyl)pyrrolidine-2-carboxylic acid (40 mg, yield 92.9%) as colorless oil, which was used directly in the next reaction. LC/MS (ESI) (m/z): 431 (M+H)⁺.

### Step 3: (2S,4R)-1-((4-benzoylbenzoyl)glycyl)-N-((R)-1-(4-carbamimidoylthiophen-2-yl)ethyl)-4-fluoro-4-(fluoromethyl)pyrrolidine-2-carboxamide (Compound 202)

To a mixture of (4-benzoylbenzoyl)glycine (40 mg, 0.10 mmol) and benzyl (4S)-4-(fluoromethyl)-4-methylpyrrolidine-2-carboxylate (20 mg, 0.12 mmol) in DMF (1.0 mL) was added DIPEA (77 mg, 0.60 mmol) and PyBop (78 mg, 0.15 mmol) at 0° C. and the mixture was stirred under N₂ atmosphere at room temperature for 1 hour. The mixture was diluted with water and extracted with EtOAc twice. The combined organic layers were washed with brine, dried over anhydrous Na₂SO₄, filtered and concentrated under reduced pressure to dryness. The residue was purified by flash chromatography on silica gel (DCM:MeOH=12:1) to give Compound 202 (5.5 mg, yield 9.4%) as a white solid. ¹H NMR (400 MHz, CD₃OD) δ 8.53 (s, 1H), 8.25 (d, J=1.5 Hz, 1H), 8.22 (d, J=1.6 Hz, 1H), 8.01 (d, J=1.8 Hz, 1H), 7.99 (s, 1H), 7.86 (s, 1H), 7.84 (s, 1H), 7.81 (s, 1H), 7.79 (d, J=1.4 Hz, 1H), 7.67 (d, J=7.4 Hz, 1H), 7.57 (s, 1H), 7.55-7.54 (m, 1H), 5.27 (q, J=7.1 Hz, 1H), 4.80-4.73 (m, 2H), 4.69-4.64 (m, 2H), 4.35 (d, J=16.7 Hz, 1H), 4.14 (d, J=6.0 Hz, 1H), 3.91 (dd, J=14.5, 6.6 Hz, 1H), 2.65-2.56 (m, 1H), 2.27-2.17 (m, 1H), 1.58 (d, J=7.0 Hz, 3H). LC/MS (ESI) (m/z): 582 (M+H)⁺.

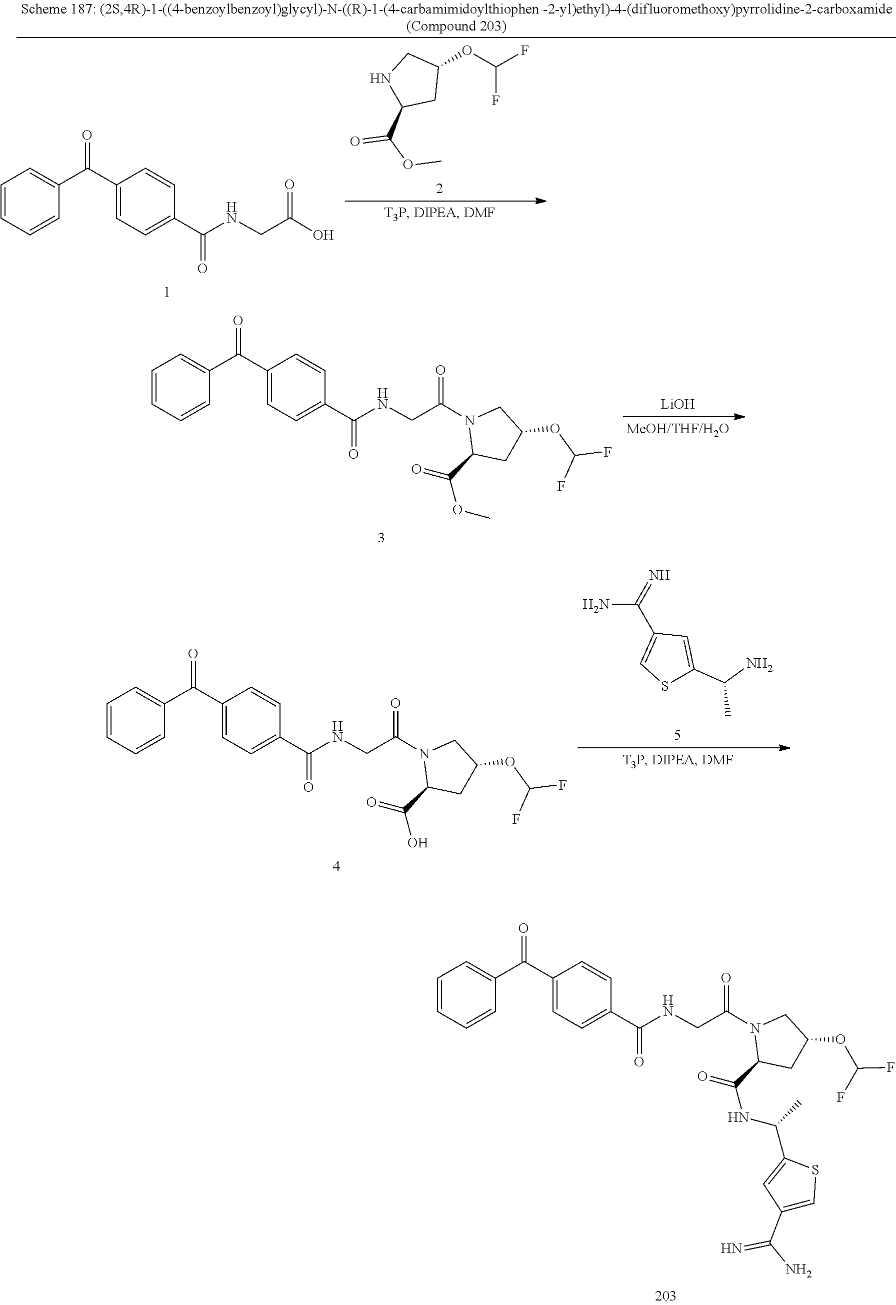

### Step 1: (2S,4R)-1-((4-benzoylbenzoyl)glycyl)-N-((R)-1-(4-carbamimidoylthiophen-2-yl)ethyl)-4-(difluoromethoxy)pyrrolidine-2-carboxamide

To a mixture of (4-benzoylbenzoyl)glycine (116 mg, 0.41 mmol) and methyl (2S,4R)-4-(difluoromethoxy)pyrrolidine-2-carboxylate (80 mg, 0.41 mmol) in DMF (1.5 mL) was added DIPEA (264 mg, 2.05 mmol) and T₃P (170 mg, 0.53 mmol) at 0° C. and the mixture was stirred under N₂ atmosphere at room temperature for 1 hour. The mixture was quenched with saturated NaHCO₃ solution and extracted twice with EtOAc. The combined organic layers were washed with brine, dried over anhydrous Na₂SO₄, filtered, and concentrated to dryness under reduced pressure. The residue was purified by flash chromatography on silica gel (PE:EtOAc=1:1) to give methyl (2S,4R)-1-((4-benzoylbenzoyl)glycyl)-4-(difluoromethoxy)pyrrolidine-2-carboxylate (120 mg, yield 63.6%) as white solid.

### Step 2: (2S,4R)-1-((4-benzoylbenzoyl)glycyl)-4-(difluoromethoxy)pyrrolidine-2-carboxylic acid

To a solution of methyl (2S,4R)-1-((4-benzoylbenzoyl)glycyl)-4-(difluoromethoxy)pyrrolidine-2-carboxylate (120 mg, 0.26 mmol) in MeOH (1 mL) and water (1 mL) was added LiOH (63 mg, 1.6 mmol) at 0° C. and the mixture was stirred at room temperature for 2 hours. The mixture was acidified with 1N aq.HCl to pH3, extracted with EtOAc twice. The combined organic layers were washed with brine, dried over anhydrous Na₂SO₄, filtered and concentrated under reduced pressure to dryness to give (2S,4R)-1-((4-benzoylbenzoyl)glycyl)-4-(difluoromethoxy)pyrrolidine-2-carboxylic acid (110 mg, yield 94.4%) as colorless oil, which was used directly in the next step. LC/MS (ESI) (m/z): 446 (M+H)⁺.

### Step 3: (2S,4R)-1-((4-benzoylbenzoyl)glycyl)-N-((R)-1-(4-carbamimidoylthiophen-2-yl)ethyl)-4-(difluoromethoxy)pyrrolidine-2-carboxamide (Compound 203)

To a mixture of (2S,4R)-1-((4-benzoylbenzoyl)glycyl)-4-(difluoromethoxy)pyrrolidine-2-carboxylic acid (45 mg, 0.10 mmol) and (R)-5-(1-aminoethyl)thiophene-3-carboximidamide (17 mg, 0.10 mmol) in DMF (1.0 mL) was added DIPEA (65 mg, 0.50 mmol) and T₃P (48 mg, 0.15 mmol) at 0° C. and the mixture was stirred under N₂ atmosphere at room temperature for 1 hour. The mixture was quenched with saturated NaHCO₃ solution and extracted with AcOEt twice. The combined organic layers were washed with brine, dried over anhydrous Na₂SO₄, filtered and concentrated to dryness under reduced pressure. The residue was purified by flash chromatography on silica gel (DCM:MeOH=12:1) and prep-HPLC to give Compound 203 (1.5 mg, yield 2.5%) as a white solid. ¹H NMR (400 MHz, CD₃OD) δ 8.55 (s, 1H), 8.23 (dd, J=12.1, 1.5 Hz, 1H), 7.99 (t, J=8.7 Hz, 2H), 7.88-7.76 (m, 4H), 7.67 (d, J=7.4 Hz, 1H), 7.54 (dd, J=12.8, 4.6 Hz, 3H), 6.50 (td, J=74.5, 17.2 Hz, 1H), 5.35-5.22 (m, 1H), 5.03 (s, 1H), 4.60 (d, J=7.9 Hz, 1H), 4.23 (dt, J=22.5, 11.3 Hz, 2H), 4.00-3.86 (m, 2H), 2.52-2.36 (m, 1H), 2.28-2.15 (m, 1H), 1.62 (dd, J=31.7, 7.0 Hz, 3H). LC/MS (ESI) (m/z): 598 (M+H)⁺.

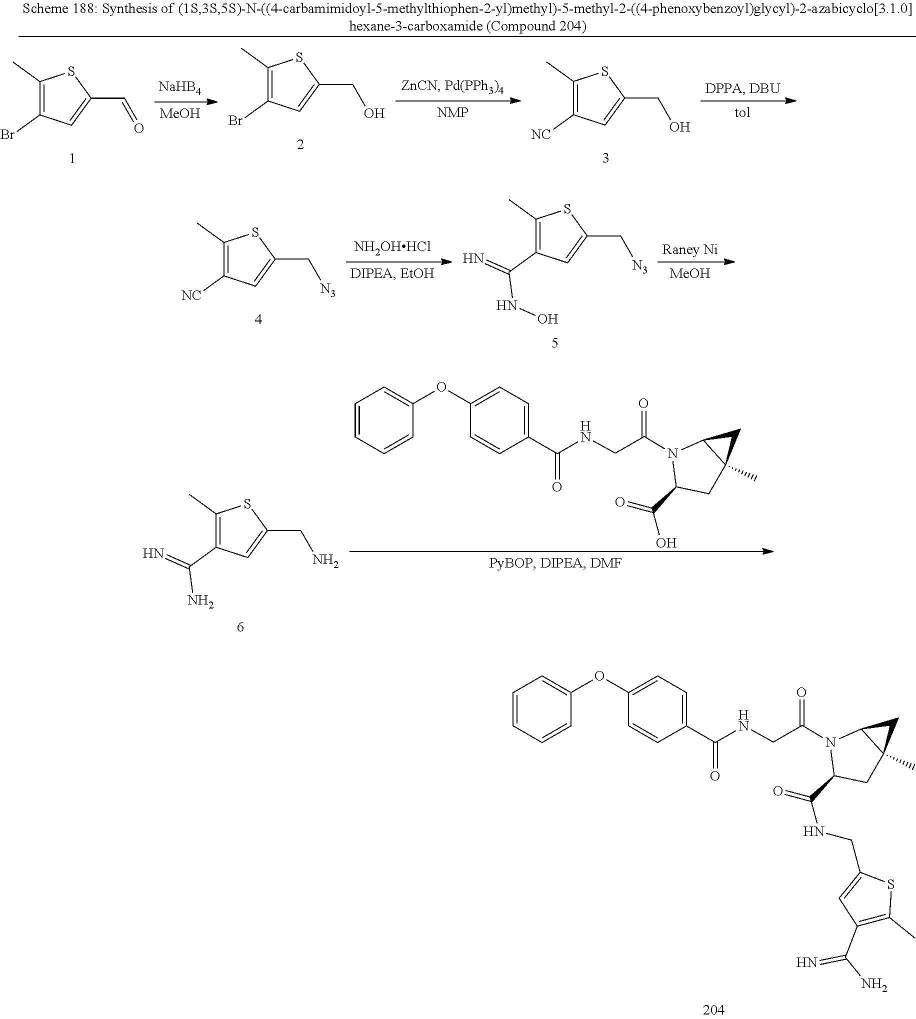

### Step 1: (4-bromo-5-methylthiophen-2-yl)methanol

At 0° C., to a solution of 4-bromo-5-methylthiophene-2-carbaldehyde (1.0 g, 4.90 mmol) in MeOH (10 mL) was added NaBH₄ (0.37 g, 9.80 mmol) in portions under N₂ atmosphere and the reaction mixture was stirred at 25° C. for 1 hour. The mixture was quenched with saturated NH₄Cl solution and extracted with EtOAc twice. The combined organic layers were washed with brine, dried over anhydrous Na₂SO₄, filtered and concentrated under reduced pressure to dryness. The residue was purified by flash chromatography on silica gel (petroleum ether:EtOAc=10:1) to afford (4-bromo-5-methylthiophen-2-yl)methanol (580 mg, yield 57.5%) as yellow solid. LC/MS (ESI) m/z: 207/209 (M+H)⁺.

### Step 2: 5-(hydroxymethyl)-2-methylthiophene-3-carbonitrile

To a solution of (4-bromo-5-methylthiophen-2-yl)methanol (400 mg, 1.94 mmol) in NMP (6 mL) was added Zn(CN)₂ (500 mg, 4.27 mmol) and Pd(PPh₃)₄ (336 mg, 0.29 mmol), the mixture was degassed under N₂ atmosphere for three times and stirred at 160° C. for 4 hours. The mixture was diluted with EtOAc, washed with water and brine, dried over anhydrous Na₂SO₄, filtered and concentrated to dryness. The residue was purified by column chromatography on silica gel (PE:EtOAc=20:1) to afford 5-(hydroxymethyl)-2-methylthiophene-3-carbonitrile (270 mg, yield 90.9%) as light oil. LC/MS (ESI) m/z: 154 (M+H)⁺.

### Step 3: 5-(azidomethyl)-2-methylthiophene-3-carbonitrile

At 0° C., to a solution of 5-(hydroxymethyl)-2-methylthiophene-3-carbonitrile (270 mg, 1.76 mmol) in toluene (6 mL) was added DBU (670 mg, 4.41 mmol) and DPPA (970 mg, 3.52 mmol) under N₂ atmosphere and the reaction mixture was stirred at 0° C. for 1.5 hours. The mixture was quenched with ice-water and extracted with EtOAc twice. The combined organic layers were washed with brine, dried over anhydrous Na₂SO₄, filtered and concentrated under reduced pressure to dryness. The residue was purified by flash chromatography on silica gel (petroleum ether:EtOAc=40:1) to afford 5-(azidomethyl)-2-methylthiophene-3-carbonitrile (280 mg, yield 89.2%) as light oil. LC/MS (ESI) m/z: 179 (M+H)⁺.

### Step 4: 5-(azidomethyl)-N-hydroxy-2-methylthiophene-3-carboximidamide

To a solution of 5-(azidomethyl)-2-methylthiophene-3-carbonitrile (280 mg, 1.57 mmol) in EtOH (8 mL) was added DIPEA (0.78 mL, 4.71 mmol) and NH₂OH·HCl (273 mg, 3.93 mmol) under N₂ atmosphere and the mixture was stirred at 30° C. for 16 hours. The mixture was diluted with DCM, washed with water and brine, dried over anhydrous Na₂SO₄, filtered and concentrated under reduced pressure to dryness. The residue was purified by flash chromatography on silica gel (petroleum ether:EtOAc=3:1) to afford 5-(azidomethyl)-N-hydroxy-2-methylthiophene-3-carboximidamide (270 mg, yield 81.3%) as light oil. LC/MS (ESI) m/z: 212 (M+H)⁺.

### Step 5: 5-(aminomethyl)-2-methylthiophene-3-carboximidamide

To a solution of 5-(azidomethyl)-N-hydroxy-2-methylthiophene-3-carboximidamide (70 mg, 0.33 mmol) in MeOH (3 mL) and AcOH (0.01 mL) was added Raney Ni (20 mg), the mixture was degassed under N₂ atmosphere for three times and stirred under a H₂ balloon at 30° C. for 16 hours. The mixture was filtered and the filtrate was concentrated under reduce pressure to afford 5-(aminomethyl)-2-methylthiophene-3-carboximidamide (40 mg, yield 71.4%) as white solid, which was used directly in the next reaction without further purification. LC/MS (ESI) (m/z): 170 (M+H)⁺.

### Step 6: (1S,3S,5S)-N-((4-carbamimidoyl-5-methylthiophen-2-yl)methyl)-5-methyl-2-((4-phenoxybenzoyl)glycyl)-2-azabicyclo[3.1.0]hexane-3-carboxamide (Compound 204)

To a mixture of (1 S,3S,5S)-5-methyl-2-((4-phenoxybenzoyl)glycyl)-2-azabicyclo[3.1.0]hexane-3-carboxylic acid (47 mg, 0.12 mmol) and 5-(aminomethyl)-2-methylthiophene-3-carboximidamide (20 mg, 0.12 mmol) in DMF (2 mL) was added DIPEA (0.1 mL, 0.60 mmol) and PyBOP (94 mg, 0.18 mmol) at 0° C. under N₂ atmosphere and the mixture was stirred at room temperature for 1 hour. The mixture was diluted with CHCl₃/i-PrOH (3/1, v/v) twice, washed with water and brine, dried over anhydrous Na₂SO₄, filtered and concentrated under reduced pressure to dryness. The residue was purified by prep-HPLC to afford Compound 204 (5.0 mg, yield 7.8%) as white solid. ¹H NMR (400 MHz, CD₃OD) δ 7.88-7.84 (m, 2H), 7.46-7.39 (m, 2H), 7.23-7.19 (m, 1H), 7.08-7.00 (m, 5H), 4.83-4.79 (m, 1H), 4.50-4.42 (m, 2H), 4.39-4.28 (m, 2H), 3.50-3.39 (m, 1H), 2.57 (d, J=5.1 Hz, 3H), 2.45-2.35 (m, 1H), 2.18-2.14 (m, 1H), 1.30 (s, 3H), 1.12 (dd, J=5.6, 5.6 Hz, 1H), 0.82-0.69 (m, 1H). LC/MS (ESI) (m/z): 546 (M+H)⁺.

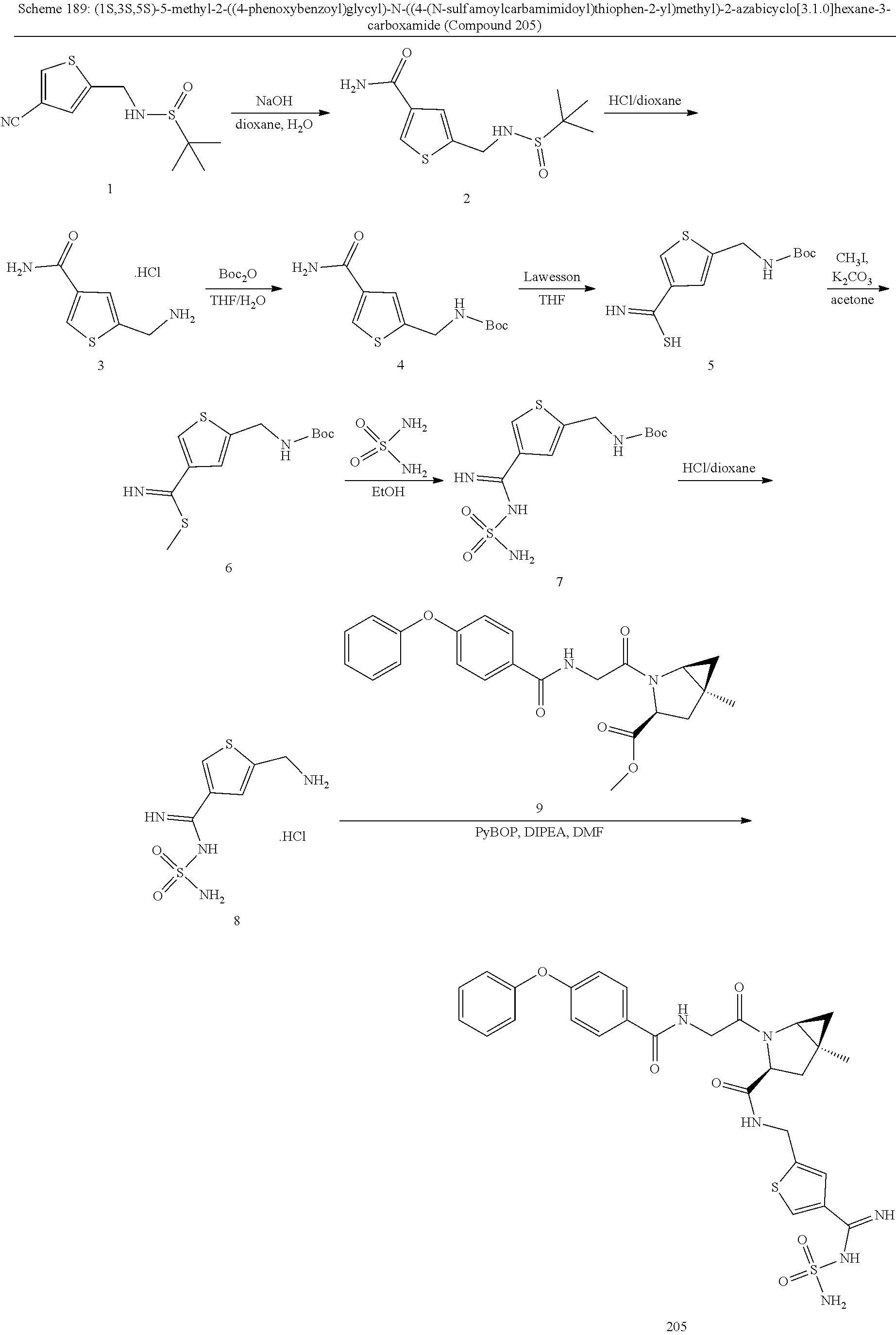

### Step 1: 5-(((tert-butylsulfinyl)amino)methyl)thiophene-3-carboxamide

To a solution of N-((4-cyanothiophen-2-yl)methyl)-2-methylpropane-2-sulfinamide (1.0 g, 4.1 mmol) in 1,4-dioxane (7.0 mL) and water (3.0 mL) was added NaOH (82 mg, 2.1 mmol). The mixture was degassed under N₂ atmosphere. The mixture was stirred at 140° C. for 4 hours. The mixture was diluted with water and extracted with AcOEt twice. The combined organic layers were dried over anhydrous Na₂SO₄, filtered and concentrated to dryness under reduced pressure. The residue was purified by column chromatography on silica gel (PE:AcOEt=1:1) to give 5-(((tert-butylsulfinyl)amino)methyl)thiophene-3-carboxamide (400 mg, yield 37.3%) as yellow solid. LC/MS (ESI) m/z: 261 (M+H)⁺.

### Step 2: 5-(aminomethyl)thiophene-3-carboxamide

A mixture of 5-(((tert-butylsulfinyl)amino)methyl)thiophene-3-carboxamide (400 mg, 1.5 mmol) in HCl/1,4-dioxane (4.0 mL) was stirred under N₂ atmosphere at room temperature for 30 minutes. The reaction mixture was concentrated to dryness under reduced pressure, diluted with DCM again and dried under vacuum to give 5-(aminomethyl)thiophene-3-carboxamide hydrochloride (230 mg, yield 95.7%), which was used directly in the next step without further purification. LC/MS (ESI) m/z: 296 (M+H)⁺.

### Step 3: tert-butyl ((4-carbamoylthiophen-2-yl)methyl)carbamate

To a solution of 5-(aminomethyl)thiophene-3-carboxamide (230 mg, 1.47 mmol) in THE (2.0 mL) and water (1.0 mL) was added Boc₂O (960 mg, 4.4 mmol) under N₂ atmosphere at 0° C. and the mixture was stirred at 25° C. for overnight. The mixture was diluted with water and extracted with AcOEt twice.

The combined organic layers were washed with brine, dried over anhydrous Na₂SO₄, filtered and concentrated to dryness under reduced pressure. The residue was purified by column chromatography on silica gel (PE:AcOEt=1:1) to give tert-butyl ((4-carbamoylthiophen-2-yl)methyl)carbamate (240 mg, yield 65.7%) as yellow oil. LCMS (ESI) m/z=257 (M+H)⁺.

### Step 4: 5-(((tert-butoxycarbonyl)amino)methyl)thiophene-3-carbimidothioic acid

To a solution of tert-butyl ((4-carbamoylthiophen-2-yl)methyl)carbamate (240 mg, 0.67 mmol) in THE (3.0 mL) was added Lawesson reagent (327 mg, 0.81 mmol) under N₂ atmosphere at 0° C. and the mixture was stirred at 25° C. for overnight. The mixture was diluted with water and extracted with AcOEt twice. The combined organic layers were washed with brine, dried over anhydrous Na₂SO₄, filtered and concentrated to dryness under reduced pressure. The residue was purified by column chromatography on silica gel (DCM:MeOH=15:1) to give 5-(((tert-butoxycarbonyl)amino)methyl)thiophene-3-carbimidothioic acid (110 mg, yield 57.9%) as yellow oil. LCMS (ESI) m/z=273 (M+H)⁺.

### Step 5: methyl 5-(((tert-butoxycarbonyl)amino)methyl)thiophene-3-carbimidothioate

To a solution of 5-(((tert-butoxycarbonyl)amino)methyl)thiophene-3-carbimidothioic acid (110 mg, 0.30 mmol) in acetone (1.5 mL) was added CH₃I (126 mg, 0.90 mmol) under N₂ atmosphere at 0° C. and the mixture was stirred at 25° C. for overnight. The mixture was diluted with water and extracted with AcOEt twice. The combined organic layers were washed with brine, dried over anhydrous Na₂SO₄, filtered and concentrated to dryness under reduced pressure. The residue was purified by column chromatography on silica gel (PE:AcOEt=1:1) to give methyl 5-(((tert-butoxycarbonyl)amino)methyl)thiophene-3-carbimidothioate (110 mg, yield 95.1%) as yellow oil. LCMS (ESI) m/z=287 (M+H)⁺.

### Step 6: tert-butyl ((4-(N-sulfamoylcarbamimidoyl)thiophen-2-yl)methyl)carbamate

To a solution of methyl 5-(((tert-butoxycarbonyl)amino)methyl)thiophene-3-carbimidothioate (110 mg, 0.35 mmol) in EtOH (1.5 mL) was added sulfuric diamide (670 mg, 7.0 mmol) under N₂ atmosphere at 0° C. and the mixture was stirred at 120° C. overnight in a sealed pressure tube. The mixture was diluted with water and extracted with AcOEt twice. The combined organic layers were washed with brine, dried over anhydrous Na₂SO₄, filtered and concentrated to dryness under reduced pressure. The residue was purified by column chromatography on silica gel (PE:AcOEt=1:1) to give tert-butyl ((4-(N-sulfamoylcarbamimidoyl)thiophen-2-yl)methyl)carbamate (20 mg, yield 17.1%) as yellow solid. LCMS (ESI) m/z=335 (M+H)⁺.

### Step 7: 5-(aminomethyl)-N-sulfamoylthiophene-3-carboximidamide

To a mixture of tert-butyl ((4-(N-sulfamoylcarbamimidoyl)thiophen-2-yl)methyl)carbamate (18 mg, 53.9 umol) in HCl/1,4-dioxane (1 mL) was stirred under N₂ atmosphere at room temperature for 30 minutes. The reaction mixture was concentrated to dryness under reduced pressure, diluted with DCM again and dried under vacuum to give 5-(aminomethyl)-N-sulfamoylthiophene-3-carboximidamide hydrochloride (12 mg, yield 95.0%) as a white solid, which was used directly in the next step without further purification. LC/MS (ESI) m/z: 235 (M+H)⁺

### Step 8: (1S,3S,5S)-5-methyl-2-((4-phenoxybenzoyl)glycyl)-N-((4-(N-sulfamoylcarbamimidoyl)thiophen-2-yl)methyl)-2-azabicyclo[3.1.0]hexane-3-carboxamide (Compound 205)

To a mixture of (1 S,3S,5S)-5-methyl-2-((4-phenoxybenzoyl)glycyl)-2-azabicyclo[3.1.0]hexane-3-carboxylic acid (24 mg, 61.4 umol) and 5-(aminomethyl)-N-sulfamoylthiophene-3-carboximidamide (12 mg, 51.2 mmol) in DMF (1.0 mL) was added DIPEA (32 mg, 246 umol) and PyBOP (32 mg, 61.4 umol) at 0° C. and the mixture was stirred under N₂ atmosphere at room temperature for 1 hour. The organic layer was diluted with water, extracted with AcOEt twice and the combined organic layers were washed with brine, dried over anhydrous Na₂SO₄, filtered and concentrated to dryness under reduced pressure. The residue was purified by flash chromatography on silica gel (DCM:MeOH=12:1) to give Compound 205 (1.1 mg, yield 2.9%) as a white solid. ¹H NMR (400 MHz, CD₃OD) δ 8.09 (s, 1H), 8.02 (t, J=5.8 Hz, 1H), 7.88-7.83 (m, 2H), 7.44-7.39 (m, 3H), 7.20 (t, J=7.4 Hz, 1H), 7.07 (d, J=7.7 Hz, 2H), 7.01 (d, J=8.8 Hz, 2H), 5.34 (t, J=4.6 Hz, 1H), 4.81 (d, J=3.3 Hz, 1H), 4.51 (d, J=5.3 Hz, 1H), 4.41 (d, J=16.5 Hz, 1H), 4.28 (d, J=16.5 Hz, 1H), 3.35 (s, 15H), 2.40-2.34 (m, 1H), 1.60 (s, 3H), 1.17-1.15 (m, 1H), 0.78 (t, J=6.0 Hz, 1H). LC/MS (ESI) (m/z): 611(M+H)⁺.

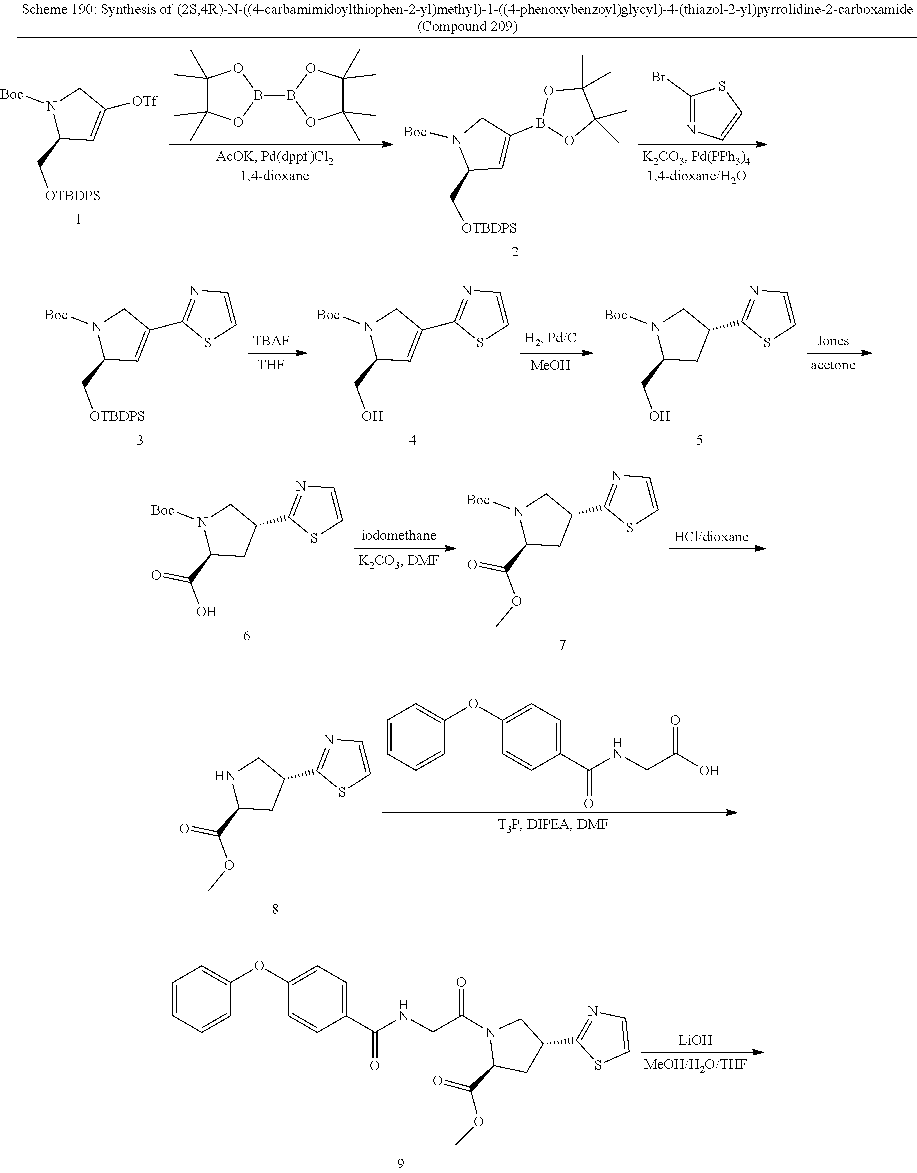

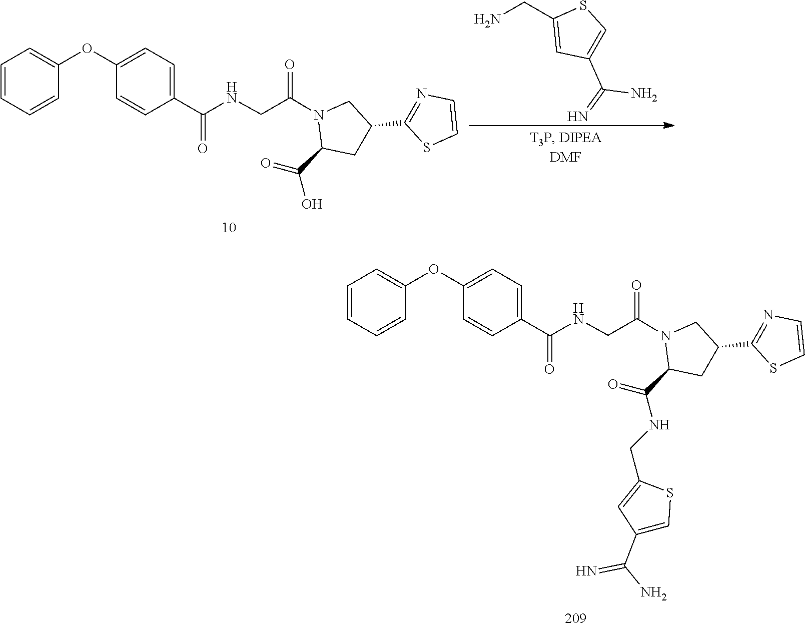 [SMILES: O=C(NCC(=O)N1C[C@H](c2nccs2)C[C@H]1C(=O)O)c1ccc(Oc2ccccc2)cc1 ; N=C(N)c1csc(CN)c1 ; N=C(N)c1csc(CNC(=O)[C@@H]2C[C@@H](c3nccs3)CN2C(=O)CNC(=O)c2ccc(Oc3ccccc3)cc2)c1]

### Step 1: tert-butyl (S)-2-(((tert-butyldiphenylsilyl)oxy)methyl)-4-(4,4,5,5-tetramethyl-1,3,2-dioxaborolan-2-yl)-2,5-dihydro-1H-pyrrole-1-carboxylate

To a mixture of tert-butyl (S)-2-(((tert-butyldiphenylsilyl)oxy)methyl)-4-(((trifluoromethyl)sulfonyl)oxy)-2,5-dihydro-1H-pyrrole-1-carboxylate (3.0 g, 5.1 mmol) and Pd(dppf)Cl₂ (261 mg, 0.36 mmol) in 1,4-dioxane (20 mL) was added 4,4,4′,4′,5,5,5′,5′-octamethyl-2,2′-bi(1,3,2-dioxaborolane) (2.6 g, 10.2 mmol) and AcOK (1.5 g, 15.4 mmol) under N₂ atmosphere at 0° C. The mixture was stirred at 80° C. overnight and was used in the next step directly without purification.

### Step 2: tert-butyl (S)-2-(((tert-butyldiphenylsilyl)oxy)methyl)-4-(thiazol-2-yl)-2,5-dihydro-1H-pyrrole-1-carboxylate

The mixture of previous step was added 2-bromothiazole (1.6 g, 10 mmol), Pd(PPh₃)₄ (400 mg, 0.35 mmol), K₂CO₃ (2.7 g, 20 mmol), 1,4-dioxane (5 mL) and water (15 mL). The new mixture was degassed under N₂ atmosphere and stirred at 100° C. for 3 hours before diluted with water and extracted with EtOAc twice. The combined organic layers were washed with brine, dried over anhydrous Na₂SO₄, filtered and concentrated to dryness under reduced pressure. The residue was purified by column chromatography on silica gel (PE:EtOAc=5:1) to give the tert-butyl (S)-2-(((tert-butyldiphenylsilyl)oxy)methyl)-4-(thiazol-2-yl)-2,5-dihydro-1H-pyrrole-1-carboxylate (980 mg, two step yield 36.7%) as colorless oil. LCMS (ESI) m/z=521(M+H)⁺.

### Step 3: tert-butyl (S)-2-(hydroxymethyl)-4-(thiazol-2-yl)-2,5-dihydro-1H-pyrrole-1-carboxylate

To a solution of tert-butyl (S)-2-(((tert-butyldiphenylsilyl)oxy)methyl)-4-(thiazol-2-yl)-2,5-dihydro-1H-pyrrole-1-carboxylate (980 mg, 1.9 mmol) in THE (10 mL) was added TBAF (4.2 mL, 4.1 mmol) at 0° C. and the mixture was stirred under N₂ atmosphere at room temperature for 16 hours. The mixture was diluted with saturated aq.NH₄Cl solution and extracted with EtOAc twice. The combined organic layers were washed with brine, dried over anhydrous Na₂SO₄, filtered and concentrated to dryness under reduced pressure. The residue was purified by column chromatography on silica gel (PE:EtOAc=10:1) to give tert-butyl (S)-2-(hydroxymethyl)-4-(thiazol-2-yl)-2,5-dihydro-1H-pyrrole-1-carboxylate (420 mg, yield 79.2%) as yellow oil. LCMS (ESI) m/z=283 (M+H)⁺.

### Step 4: tert-butyl (2S,4R)-2-(hydroxymethyl)-4-(thiazol-2-yl)pyrrolidine-1-carboxylate

To a solution of tert-butyl (S)-2-(hydroxymethyl)-4-(thiazol-2-yl)-2,5-dihydro-1H-pyrrole-1-carboxylate (420 mg, 1.5 mmol) in MeOH (5 mL) was added Pd/C (140 mg, 10% wt.) at room temperature and the mixture was degassed under N₂ atmosphere for three times and stirred under a H₂ balloon at room temperature overnight. The mixture was filtered and the filtrate was concentrated to dryness under reduced pressure to give tert-butyl (2S,4R)-2-(hydroxymethyl)-4-(thiazol-2-yl)pyrrolidine-1-carboxylate (366 mg, yield 86.4%) as a colorless oil, which was used directly in the next step. LC/MS (ESI) (m/z): 285(M+H)⁺.

### Step 5: (2S,4R)-1-(tert-butoxycarbonyl)-4-(thiazol-2-yl)pyrrolidine-2-carboxylic acid

To a mixture of tert-butyl (S)-2-(hydroxymethyl)-4-(thiazol-2-yl)-2,5-dihydro-1H-pyrrole-1-carboxylate (366 mg, 1.3 mmol) in acetone (4.0 mL) was added Jones oxidant (0.7 mL) and stirred under N₂ atmosphere at room temperature for 20 minutes. The mixture was quenched with saturated aq.Na₂S₂O₃ solution and extracted with EtOAc twice. The combined organic layers were washed with brine, dried over anhydrous Na₂SO₄, filtered and concentrated to dryness under reduced pressure.

The residue was purified by column chromatography on silica gel (PE:EtOAc=3:1) to give (2S,4R)-1-(tert-butoxycarbonyl)-4-(thiazol-2-yl)pyrrolidine-2-carboxylic acid (220 mg, yield 57.1%) as yellow oil.

LCMS (ESI) m/z=299(M+H)⁺.

### Step 6: 1-(tert-butyl) 2-methyl (2S,4R)-4-(thiazol-2-yl)pyrrolidine-1,2-dicarboxylate

To a mixture of (2S,4R)-1-(tert-butoxycarbonyl)-4-(thiazol-2-yl)pyrrolidine-2-carboxylic acid (120 mg, 0.39 mmol) and K₂CO₃ (216 mg, 1.6 mmol) in DMF (2.0 mL) was added iodomethane (223 mg, 1.6 mmol) under N₂ atmosphere at room temperature and stirred overnight. The mixture was diluted with water and extracted with EtOAc twice. The combined organic layers were washed with brine, dried over anhydrous Na₂SO₄, filtered, and concentrated to dryness under reduced pressure. The residue was purified by column chromatography on silica gel (PE:EtOAc=3:1) to give 1-(tert-butyl) 2-methyl (2S,4R)-4-(thiazol-2-yl)pyrrolidine-1,2-dicarboxylate (136 mg, yield 58.8%) as yellow oil. LCMS (ESI) m/z=313 (M+H)⁺.

### Step 7: methyl (2S,4R)-4-(thiazol-2-yl)pyrrolidine-2-carboxylate

To a mixture of 1-(tert-butyl) 2-methyl (2S,4R)-4-(thiazol-2-yl)pyrrolidine-1,2-dicarboxylate (136 mg, 0.43 mmol) in HCl/1,4-dioxane (1.5 mL) was stirred under N₂ atmosphere at room temperature for 30 minutes. The reaction mixture was concentrated to dryness under reduced pressure, diluted with DCM again and dried under vacuum to give methyl (2S,4R)-4-(thiazol-2-yl)pyrrolidine-2-carboxylate (82 mg, yield 89.2%), which was used directly in the next step without further purification. LC/MS (ESI) m/z: 213 (M+H)⁺.

### Step 8: methyl (2S,4R)-1-((4-phenoxybenzoyl)glycyl)-4-(thiazol-2-yl)pyrrolidine-2-carboxylate

To a mixture of methyl (2S,4R)-4-(thiazol-2-yl)pyrrolidine-2-carboxylate (82 mg, 0.39 mmol) and (4-phenoxybenzoyl)glycine (115 mg, 0.42 mmol) in DMF (1.5 mL) was added DIPEA (273 mg, 2.1 mmol) and T₃P (175 mg, 0.55 mmol, 50% wt. in EtOAc) at 0° C. and the mixture was stirred under N₂ atmosphere at room temperature for 2 hours. The organic layer was quenched with saturated aq.NaHCO₃ solution and extracted with EtOAc twice. The combined organic layers were washed with brine, dried over anhydrous Na₂SO₄, filtered, and concentrated to dryness under reduced pressure. The residue was purified by flash chromatography on silica gel (PE:EtOAc=3:1) to give methyl (2S,4R)-1-((4-phenoxybenzoyl)glycyl)-4-(thiazol-2-yl)pyrrolidine-2-carboxylate (120 mg, yield 60.8%) as white solid. LC/MS (ESI) (m/z): 466(M+H)⁺.

### Step 9: (2S,4R)-1-((4-phenoxybenzoyl)glycyl)-4-(thiazol-2-yl)pyrrolidine-2-carboxylic acid

To a solution of methyl (2S,4R)-1-((4-phenoxybenzoyl)glycyl)-4-(thiazol-2-yl)pyrrolidine-2-carboxylate (60 mg, 0.13 mmol) in MeOH (1.0 mL), THE (0.5 mL) and water (0.5 mL) was added LiOH (31 mg, 0.78 mmol) at 0° C. and the mixture was stirred at room temperature for 1 hour. The mixture was acidified with 1N aq.HCl to pH=3, extracted with AcOEt twice. The combined organic layers were washed with brine, dried over anhydrous Na₂SO₄, filtered and concentrated under reduced pressure to dryness to give (2S,4R)-1-((4-phenoxybenzoyl)glycyl)-4-(thiazol-2-yl)pyrrolidine-2-carboxylic acid (40 mg, yield 68.5%) as colorless oil, which was used directly in the next step. LC/MS (ESI) (m/z): 452 (M+H)⁺.

### Step 10: (2S,4R)-N-((4-carbamimidoylthiophen-2-yl)methyl)-1-((4-phenoxybenzoyl)glycyl)-4-(thiazol-2-yl)pyrrolidine-2-carboxamide (Compound 209)

To a mixture of (2S,4R)-1-((4-phenoxybenzoyl)glycyl)-4-(thiazol-2-yl)pyrrolidine-2-carboxylic acid (40 mg, 0.089 mmol) and 5-(aminomethyl)thiophene-3-carboximidamide (21 mg, 0.13 mmol) in DMF (1.3 mL) was added DIPEA (69 mg, 0.53 mmol) and T₃P (37 mg, 0.12 mmol, 50% wt. in EtOAc) at 0° C. and the mixture was stirred under N₂ atmosphere at room temperature for 2 hours. The organic layer was quenched with saturated aq.NaHCO₃ solution and extracted with EtOAc twice. The combined organic layers were washed with brine, dried over anhydrous Na₂SO₄, filtered and concentrated to dryness under reduced pressure. The residue was purified by pre-HPLC to give Compound 209 (2.8 mg, yield 5.4%) as white solid. LC/MS (ESI) (m/z): 589(M+H)⁺. ¹HNMR (400 MHz, CD₃OD) δ 8.53 (s, 2H), 8.21 (t, J=8.7 Hz, 1H), 7.87-7.83 (m, 2H), 7.72 (dd, J=14.8, 3.3 Hz, 1H), 7.52 (dd, J=11.8, 3.4 Hz, 1H), 7.42 (t, J=8.0 Hz, 3H), 7.20 (t, J=7.4 Hz, 1H), 7.06 (d, J=7.7 Hz, 2H), 7.00 (d, J=8.8 Hz, 2H), 4.59-4.51 (m, 3H), 4.36-4.30 (m, 1H), 4.29-4.15 (m, 2H), 4.05-3.91 (m, 2H), 2.31 (dd, J=22.1, 9.6 Hz, 1H), 2.15 (dd, J=38.3, 30.6 Hz, 1H). LC/MS (ESI) (m/z): 589 (M+H)⁺.

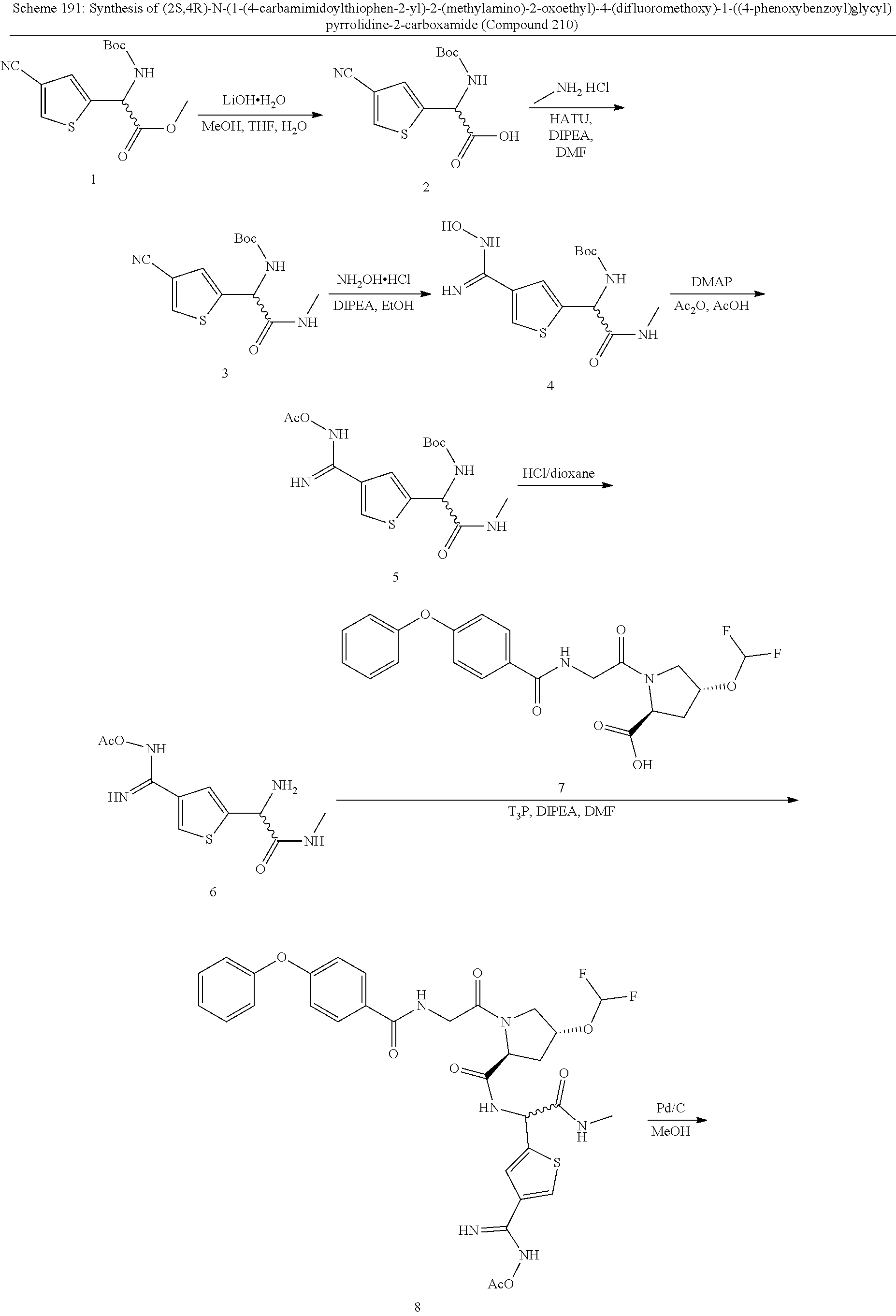

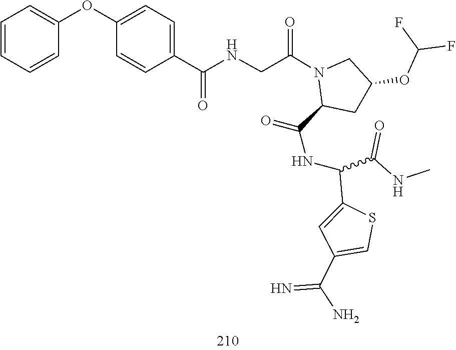 [SMILES: CNC(=O)C(NC(=O)[C@@H]1C[C@@H](OC(F)F)CN1C(=O)CNC(=O)c1ccc(Oc2ccccc2)cc1)c1cc(C(=N)N)cs1]

### Step 1: 2-((tert-butoxycarbonyl)amino)-2-(4-cyanothiophen-2-yl)acetic acid (2)

To a solution of methyl 2-((tert-butoxycarbonyl)amino)-2-(4-cyanothiophen-2-yl)acetate (150 mg, 0.51 mmol) in MeOH (2 mL), THE (1 mL) and water (1 mL) was added LiOH·H₂O (64.3 mg, 1.53 mmol) at 0° C. and the mixture was stirred under N₂ atmosphere at room temperature for 3 hours. The mixture was acidified with 1N aq.HCl solution to pH-3 and extracted with ethyl acetate twice. The combined organic layers were washed with brine, dried over Na₂SO₄, filtered and concentrated to dryness under reduced pressure to give 2-((tert-butoxycarbonyl)amino)-2-(4-cyanothiophen-2-yl)acetic acid (138 mg, yield 96.2%) as colorless oil, which was used directly in next step without further purification. LC/MS (ESI) (m/z): 283 (M+H)⁺.

### Step 2: tert-butyl (1-(4-cyanothiophen-2-yl)-2-(methylamino)-2-oxoethyl)carbamate (3)

To a mixture of 2-((tert-butoxycarbonyl)amino)-2-(4-cyanothiophen-2-yl)acetic acid (130 mg, 0.46 mmol) and methanamine hydrochloride (38 mg, 0.55 mmol) in DMF (2 mL) was added DIPEA (356 mg, 2.76 mmol) and HATU (524 mg, 1.38 mmol) at 0° C. under N₂ atmosphere and the mixture was stirred at room temperature for 2 hours. The mixture was diluted with ethyl acetate, washed with water and brine, dried over anhydrous Na₂SO₄, filtered and concentrated to dryness. The residue was purified by flash chromatography on silica gel (PE:EtOAc=1:1) to afford tert-butyl (1-(4-cyanothiophen-2-yl)-2-(methylamino)-2-oxoethyl)carbamate (100 mg, yield 73.8%) as yellow oil. LC/MS (ESI) m/z: 296(M+H)⁺

### Step 3: tert-butyl (1-(4-(N-hydroxycarbamimidoyl)thiophen-2-yl)-2-(methylamino)-2-oxoethyl)carbamate (4)

To a mixture of tert-butyl (1-(4-cyanothiophen-2-yl)-2-(methylamino)-2-oxoethyl)carbamate (100 mg, 0.34 mmol) in EtOH (2 mL) was added DIPEA (132 mg, 1.02 mmol) and NH₂OH·HCl (54.8 mg, 0.85 mmol) and the mixture was stirred at 25° C. for 16 hours. The mixture was diluted with DCM, washed with water and brine, dried over anhydrous Na₂SO₄, filtered and concentrated to dryness. The residue was purified by prep-TLC (DCM:MeOH=10:1) to give tert-butyl (1-(4-(N-hydroxycarbamimidoyl)thiophen-2-yl)-2-(methylamino)-2-oxoethyl)carbamate (100 mg, yield 90%) as colorless oil. LC/MS (ESI) m/z: 329 (M+H)⁺.

### Step 4: tert-butyl (1-(4-(N-acetoxycarbamimidoyl)thiophen-2-yl)-2-(methylamino)-2-oxoethyl)carbamate (5)

To a mixture of tert-butyl (1-(4-(N-hydroxycarbamimidoyl)thiophen-2-yl)-2-(methylamino)-2-oxoethyl)carbamate (100 mg, 0.30 mmol) in AcOH (2 mL) was added Ac₂O (2 mL) and DMAP (5 mg) and the mixture was stirred at 25° C. for 16 hours. The mixture was diluted with ethyl acetate, washed with water and brine, dried over anhydrous Na₂SO₄, filtered and concentrated to dryness. The residue was purified by flash chromatography on silica gel (DCM:MeOH=10:1) to afford tert-butyl (1-(4-(N-acetoxycarbamimidoyl)thiophen-2-yl)-2-(methylamino)-2-oxoethyl) carbamate (83 mg, yield 73.6%) as yellow oil. LC/MS (ESI) m/z: 371(M+H)⁺.

### Step 5: 2-(4-(N-acetoxycarbamimidoyl)thiophen-2-yl)-2-amino-N-methylacetamide (6)

A mixture of tert-butyl (1-(4-(N-acetoxycarbamimidoyl)thiophen-2-yl)-2-(methylamino)-2-oxoethyl)carbamate (80 mg, 0.22 mmol) in HCl/1,4-dioxane (3 mL) was stirred at room temperature for 1 hour. The reaction mixture was concentrated to dryness under reduced pressure, co-evaporated with DCM and dried under vacuum to give (R)-5-(amino(cyclopentyl)methyl)thiophene-3-carboximidamide hydrochloride (56 mg, yield 95.8%) as colorless oil, which was used directly in the next reaction without further purification. LC/MS (ESI) m/z: 271 (M+H)⁺.

### Step 6: (2S,4R)-N-(1-(4-(N-acetoxycarbamimidoyl)thiophen-2-yl)-2-(methylamino)-2-oxoethyl)-4-(difluoromethoxy)-1-((4-phenoxybenzoyl)glycyl)pyrrolidine-2-carboxamide (7)

To a mixture of 2-(4-(N-acetoxycarbamimidoyl)thiophen-2-yl)-2-amino-N-methylacetamide (25 mg, 0.093 mmol) and (2S,4R)-4-(difluoromethoxy)-1-((4-phenoxybenzoyl)glycyl) pyrrolidine-2-carboxylic acid (48.4 mg, 0.112 mmol) in DMF (2 mL) was added DIPEA (60 mg, 0.465 mmol) and T₃P (71 mg, 0.112 mmol 50% wt. in EtOAc) at 0° C. under N₂ atmosphere and the mixture was stirred at 25° C. for 3 hours. The mixture was quenched with water and extracted with EtOAc twice. The combined organic layers were washed with brine, dried over anhydrous Na₂SO₄, filtered and concentrated to dryness under reduced pressure. The residue was purified by prep-TLC (DCM:MeOH=10:1) to give (2S,4R)-N-(1-(4-(N-acetoxycarbamimidoyl) thiophen-2-yl)-2-(methylamino)-2-oxoethyl)-4-(difluoromethoxy)-1-((4-phenoxybenzoyl) glycyl)pyrrolidine-2-carboxamide (34 mg, yield 52.7%) as colorless oil. LC/MS (ESI) m/z: 687 (M+H)⁺.

Step 7: (2S,4R)-N-(1-(4-carbamimidoylthiophen-2-yl)-2-(methylamino)-2-oxoethyl)-4-(difluoromethoxy)-1-((4-phenoxybenzoyl)glycyl)pyrrolidine-2-carboxamide (Compound 210) To a mixture of (2S,4R)-N-(1-(4-(N-acetoxycarbamimidoyl)thiophen-2-yl)-2-(methylamino)-2-oxoethyl)-4-(difluoromethoxy)-1-((4-phenoxybenzoyl)glycyl)pyrrolidine-2-carboxamide (34 mg, 0.049 mmol) in MeOH (2 mL) was added Pd/C (5 mg), and the mixture was degassed under N₂ atmosphere for three times and the reaction mixture was stirred at 25° C. for 2 hours. The mixture was filtered and concentrated to dryness under reduced pressure. The residue was purified by prep-HPLC to give Compound 210 (2.0 mg, yield 6.4%) as a white solid. ¹H NMR (400 MHz, CD₃OD) δ 8.53 (s, 1H), 8.30 (s, 1H), 7.86-7.82 (m, 2H), 7.57 (d, J=27.3 Hz, 1H), 7.41 (t, J=8.0 Hz, 2H), 7.20 (t, J=7.6 Hz, 1H), 7.06 (d, J=8.1 Hz, 2H), 7.00 (dd, J=8.8, 3.3 Hz, 2H), 6.52 (t, J=74.4 Hz, 1H), 5.76 (d, J=8.6 Hz, 1H), 5.03 (d, J=6.4 Hz, 1H), 4.67-4.58 (m, 1H), 4.20 (t, J=10.9 Hz, 2H), 3.98 (dd, J=11.7, 4.4 Hz, 1H), 3.89 (d, J=10.0 Hz, 1H), 2.72 (d, J=12.6 Hz, 3H), 2.57-2.41 (m, 1H), 2.31-2.24 (m, 1H). LC/MS (ESI) m/z: 629 (M+H)⁺.

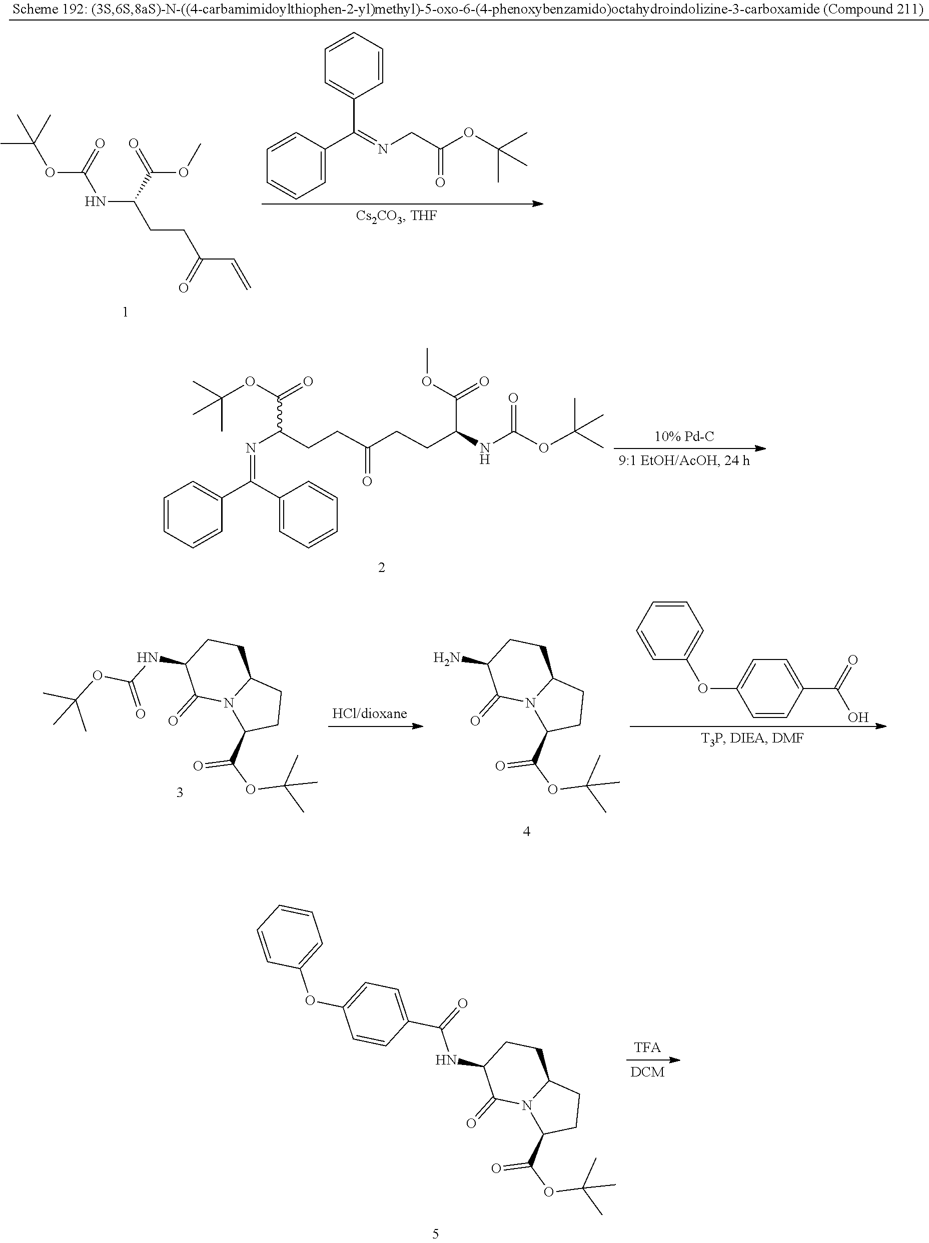

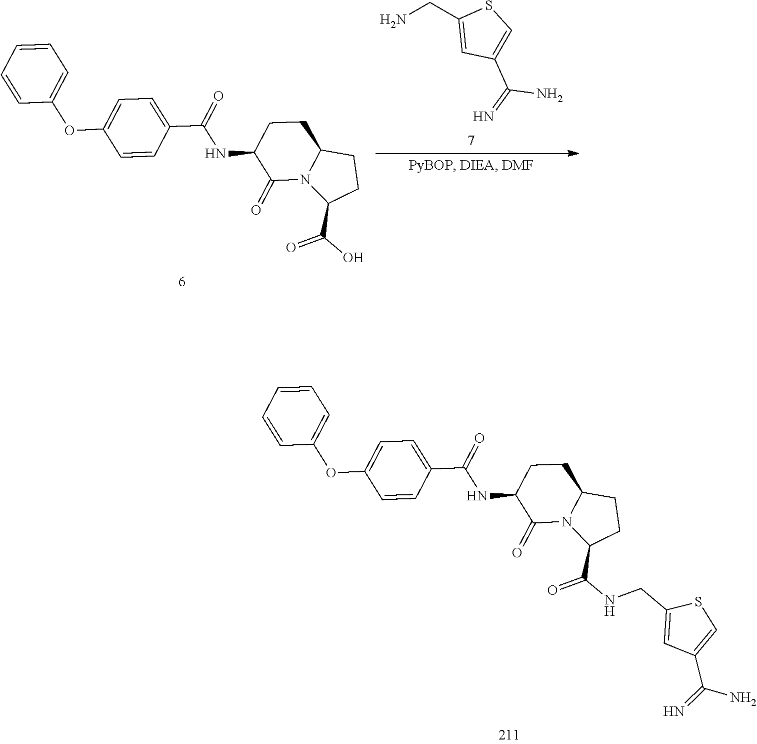 [SMILES: O=C(N[C@H]1CC[C@H]2CC[C@@H](C(=O)O)N2C1=O)c1ccc(Oc2ccccc2)cc1 ; N=C(N)c1csc(CN)c1 ; N=C(N)c1csc(CNC(=O)[C@@H]2CC[C@@H]3CC[C@H](NC(=O)c4ccc(Oc5ccccc5)cc4)C(=O)N32)c1]

### Step 1: 9-(tert-butyl) 1-methyl (2S)-2-((tert-butoxycarbonyl)amino)-8-((diphenylmethylene) amino)-5-oxononanedioate (2)

To a mixture of methyl (S)-2-((tert-butoxycarbonyl)amino)-5-oxohept-6-enoate (1 g, 0.37 mmol) and tert-butyl 2-((diphenylmethylene)amino)acetate (1.2 g, 4.05 mmol) in THE (20 mL) was added Cs₂CO₃ (1.2 g, 3.68 mmol) at 0° C. under N₂ atmosphere and the mixture was stirred at room temperature overnight. The mixture was diluted with water. The aqueous layer was extracted with EtOAc twice and the combined organic layers were washed with brine, dried over anhydrous Na₂SO₄, filtered, and concentrated to dryness under reduced pressure. The residue was purified by flash chromatography on silica gel (PE:EtOAc=5:1) to afford 9-(tert-butyl) 1-methyl (2S)-2-((tert-butoxycarbonyl)amino)-8-((diphenylmethylene) amino)-5-oxononanedioate (1.3 g, yield 65.0%) as yellow oil. LC/MS (ESI) m/z: 567 (M+H)⁺.

### Step 2: tert-butyl (3S,6S,8aS)-6-((tert-butoxycarbonyl)amino)-5-oxooctahydroindolizine-3-carboxylate (3)

To a solution of 9-(tert-butyl) 1-methyl (2S)-2-((tert-butoxycarbonyl)amino)-8-((diphenylmethylene)amino)-5-oxononanedioate (1.3 g, 2.29 mmol) in MeOH (9 mL) were added Pd/C (100 mg, 10% wt.) and AcOH (1 mL), the mixture was degassed under N₂ atmosphere for three times and stirred under a H₂ balloon at room temperature for 16 hours. The mixture was filtered and concentrated to dryness under reduced pressure. The residue was purified by flash chromatography on silica gel (PE:EtOAc=1:1) to afford tert-butyl (3S,6S,8aS)-6-((tert-butoxycarbonyl)amino)-5-oxooctahydroindolizine-3-carboxylate (307 mg, yield 37.9%) as light oil. LC/MS (ESI) m/z: 355 (M+H)⁺.

### Step 3: tert-butyl (3S,6S,8aS)-6-amino-5-oxooctahydroindolizine-3-carboxylate(4)

A solution of tert-butyl (3S,6S,8aS)-6-((tert-butoxycarbonyl)amino)-5-oxooctahydroindolizine-3-carboxylate (307 mg, 0.87 mmol) in HCl/dioxane (4 mL) was stirred at 25° C. for 2 hours. The reaction mixture was concentrated to give tert-butyl (3S,6S,8aS)-6-amino-5-oxooctahydroindolizine-3-carboxylate (200 mg, yield 90.9%) as white solid, which was used directly in the next step. LC/MS (ESI) m/z: 255 (M+H)⁺.

### Step 4: tert-butyl (3S,6S,8aS)-5-oxo-6-(4-phenoxybenzamido)octahydroindolizine-3-carboxylate (5)

To a mixture of tert-butyl (3S,6S,8aS)-6-amino-5-oxooctahydroindolizine-3-carboxylate (200 mg, 0.79 mmol) and 4-phenoxybenzoic acid (169 mg, 0.79 mmol) in DMF (3 mL) was added T₃P (1.5 g, 2.37 mmol, 50% wt. in EtOAc) and DIPEA (0.8 mL, 4.74 mmol) at 0° C. under N₂ atmosphere and the mixture was stirred at room temperature for 1 hour. The mixture was diluted with ethyl acetate and washed with saturated aq.NaHCO₃ solution. The aqueous layer was extracted with ethyl acetate and the combined organic layers were washed with brine, dried over anhydrous Na₂SO₄, filtered and concentrated to dryness under reduced pressure. The residue was purified by flash chromatography on silica gel (PE:EtOAc=1:1) to afford methyl tert-butyl (3S,6S,8aS)-5-oxo-6-(4-phenoxybenzamido)octahydroindolizine-3-carboxylate (77 mg, yield 21.7%) as yellow oil. LC/MS (ESI) m/z: 451 (M+H)⁺.

### Step 5: (3S,6S,8aS)-5-oxo-6-(4-phenoxybenzamido)octahydroindolizine-3-carboxylic acid (6)

To a solution of tert-butyl (3S,6S,8aS)-5-oxo-6-(4-phenoxybenzamido)octahydroindolizine-3-carboxylate (77 mg, 0.17 mmol) in DCM (2 mL) was added TFA (1 mL) at 0° C., and the mixture was stirred at 25° C. for 3 hours. The reaction mixture was concentrated to give (3S,6S,8aS)-5-oxo-6-(4-phenoxybenzamido)octahydroindolizine-3-carboxylic acid (60 mg, yield 89.6%) as yellow oil, which was used directly in the next step. LC/MS (ESI) m/z: 395 (M+H)⁺.

### Step 6: (3S,6S,8aS)-N-((4-carbamimidoylthiophen-2-yl)methyl)-5-oxo-6-(4-phenoxybenzamido)octahydroindolizine-3-carboxamide (Compound 211)

To a mixture of (3S,6S,8aS)-5-oxo-6-(4-phenoxybenzamido)octahydroindolizine-3-carboxylic acid (60 mg, 0.15 mmol) and 5-(aminomethyl)thiophene-3-carboximidamide (28 mg, 0.18 mmol) in DMF (2 mL) was added T₃P (171 mg, 0.23 mmol, 50% wt. in EtOAc) and DIPEA (0.1 mL, 0.75 mmol) at 0° C. under N₂ atmosphere and the mixture was stirred at room temperature for 4 hours. The mixture was diluted with ethyl acetate and washed with saturated aq.NaHCO₃ solution. The aqueous layer was extracted with ethyl acetate and the combined organic layers were washed with brine, dried over anhydrous Na₂SO₄, filtered and concentrated to dryness under reduced pressure. The residue was purified prep-HPLC to afford Compound 211 (0.5 mg, yield 0.6%) as a white solid. ¹H NMR (400 MHz, CD₃OD) δ 8.54 (s, 3H), 8.19 (d, J=1.6 Hz, 1H), 7.81 (d, J=9.2 Hz, 2H), 7.48 (s, 1H), 7.41 (t, J=16.0 Hz, 2H), 7.20 (t, J=12.8 Hz, 1H), 7.05 (d, J=8.0 Hz, 2H), 6.98 (d, J=8.8 Hz, 2H), 4.68-4.57 (m, 3H), 4.52 (d, J=15.6 Hz, 1H), 4.44 (d, J=9.3 Hz, 1H), 2.31-2.23 (m, 2H), 2.09-1.98 (m, 3H), 1.91-1.78 (m, 3H).LC/MS (ESI) m/z: 532 (M+H)⁺.

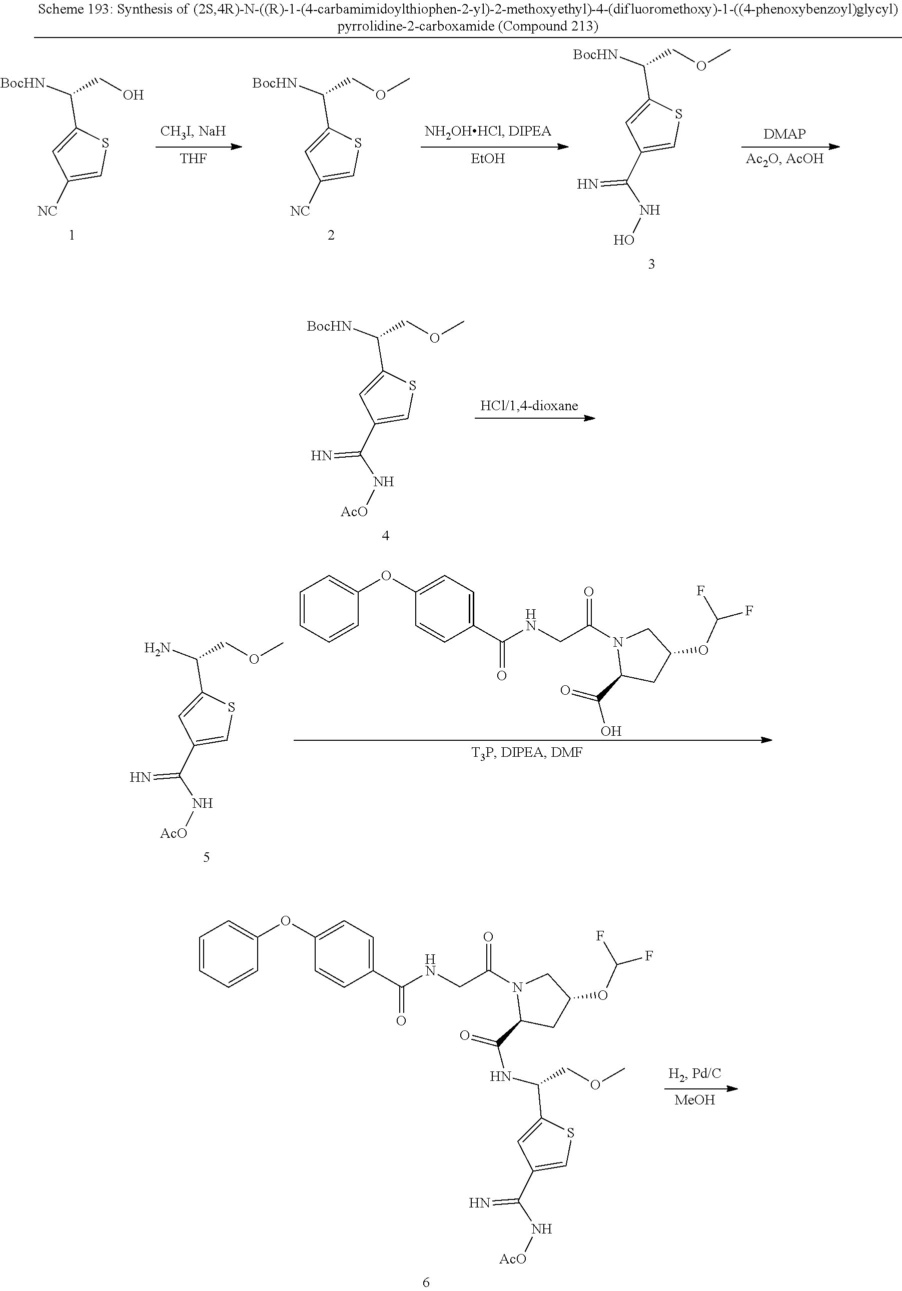

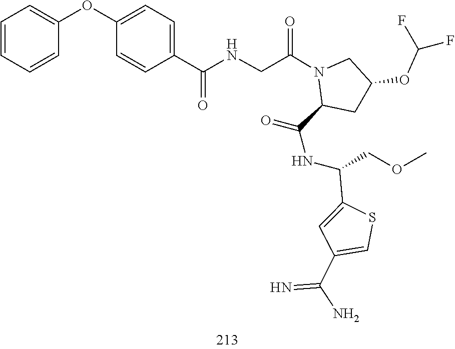 [SMILES: COC[C@@H](NC(=O)[C@@H]1C[C@@H](OC(F)F)CN1C(=O)CNC(=O)c1ccc(Oc2ccccc2)cc1)c1cc(C(=N)N)cs1]

### Step 1: tert-butyl (R)-(1-(4-cyanothiophen-2-yl)-2-methoxyethyl)carbamate (2)

To a solution of NaH (31 mg, 0.78 mmol) in THE (2 mL) was added a solution of tert-butyl (R)-(1-(4-cyanothiophen-2-yl)-2-hydroxyethyl)carbamate (100 mg, 0.37 mmol) in THE (4 mL) at 0° C. under N₂ atmosphere and the reaction mixture was stirred at room temperature for 2 hours. CH₃I (57 mg, 0.40 mmol) was added to the stirring reaction mixture. The mixture was stirred at room temperature for 1 hour and then heated up to 70° C. for 2 hours. The mixture was quenched with ice-water and extracted with EtOAc twice. The combined organic layers were washed with brine, dried over anhydrous Na₂SO₄, filtered and concentrated to dryness. The residue was purified by chromatography on silica gel (PE:EtOAc=5:1) to give tert-butyl (R)-(1-(4-cyanothiophen-2-yl)-2-methoxyethyl)carbamate (42 mg, yield 40.0%) as yellow solid. LC/MS (ESI) (m/z): 283(M+H)⁺.

### Step 2: tert-butyl (R)-(1-(4-(N-hydroxycarbamimidoyl)thiophen-2-yl)-2-methoxyethyl) carbamate (3)

To a mixture of tert-butyl (R)-(1-(4-cyanothiophen-2-yl)-2-methoxyethyl)carbamate (42 mg, 0.15 mmol) in EtOH (3 mL) was added DIPEA (58 mg, 0.45 mmol) and NH₂OH·HCl (26 mg, 0.37 mmol) and the mixture was stirred at room temperature for 16 hours. The mixture was diluted with DCM, washed with water and brine, dried over anhydrous Na₂SO₄, filtered and concentrated under reduced pressure to give tert-butyl (R)-(1-(4-(N-hydroxycarbamimidoyl)thiophen-2-yl)-2-methoxyethyl)carbamate (46 mg, yield 98.1%) as light oil, which was used directly in the next reaction without further purification. LC/MS (ESI) (m/z): 316(M+H)⁺.

### Step 3: tert-butyl (R)-(1-(4-(N-acetoxycarbamimidoyl)thiophen-2-yl)-2-methoxyethyl) carbamate (4)

To a solution of tert-butyl (R)-(1-(4-(N-hydroxycarbamimidoyl)thiophen-2-yl)-2-methoxyethyl)carbamate (46 mg, 0.15 mmol) in AcOH (1.5 mL) was added Ac₂O (1.5 mL) and DMAP (10 mg) and the mixture was stirred at 25° C. for 16 hours. The mixture was diluted with ethyl acetate, washed with water and brine, dried over anhydrous Na₂SO₄, filtered and concentrated to dryness. The residue was purified by prep-TLC (DCM:MeOH=30:1) to afford tert-butyl (R)-(1-(4-(N-acetoxycarbamimidoyl)thiophen-2-yl)-2-methoxyethyl)carbamate (22 mg, yield 42.3%) as light oil. LC/MS (ESI) m/z: 358(M+H)⁺.

### Step 4: (R)-N-acetoxy-5-(1-amino-2-methoxyethyl)thiophene-3-carboximidamide (5)

A mixture of tert-butyl (R)-(1-(4-(N-acetoxycarbamimidoyl)thiophen-2-yl)-2-methoxyethyl)carbamate (20 mg, 0.056 mmol) in HCl/1,4-dioxane (2 mL, 4 M) was stirred at room temperature for 2 hours. The reaction mixture was concentrated to dryness under reduced pressure, co-evaporated with DCM and dried under vacuum to give (R)-N-acetoxy-5-(1-amino-2-methoxyethyl)thiophene-3-carboximidamide (14 mg, yield 97.1%) as white solid, which was used directly in the next reaction without further purification. LC/MS (ESI) m/z: 258 (M+H)⁺.

### Step 5: (2S,4R)-N-((R)-1-(4-(N-acetoxycarbamimidoyl)thiophen-2-yl)-2-methoxyethyl)-4-(difluoromethoxy)-1-((4-phenoxybenzoyl)glycyl)pyrrolidine-2-carboxamide (6)

To a mixture of (2S,4R)-4-(difluoromethoxy)-1-((4-phenoxybenzoyl)glycyl)pyrrolidine-2-carboxylic acid (24 mg, 0.054 mmol) and (R)-N-acetoxy-5-(1-amino-2-methoxyethyl) thiophene-3-carboximidamide (14 mg, 0.054 mmol) in DMF (2 mL) was added DIPEA (35 mg, 0.27 mmol) and T₃P (51 mg, 0.081 mmol, 50% wt. in EtOAc) at 0° C. and the mixture was stirred at room temperature for 1 hour. The mixture was diluted with ethyl acetate and washed with saturated aq.NaHCO₃ solution. The aqueous layer was extracted with ethyl acetate and the combined organic layers were washed with brine, dried over anhydrous Na₂SO₄, filtered and concentrated to dryness under reduced pressure. The residue was purified by prep-TLC (DCM:MeOH=10:1) to give (2S,4R)-N-((R)-1-(4-(N-acetoxycarbamimidoyl)thiophen-2-yl)-2-methoxyethyl)-4-(difluoromethoxy)-1-((4-phenoxybenzoyl)glycyl)pyrrolidine-2-carboxamide (20 mg, yield 54.5%) as a white solid. LC/MS (ESI) (m/z): 674(M+H)⁺.

### Step 6: (2S,4R)-N-((R)-1-(4-carbamimidoylthiophen-2-yl)-2-methoxyethyl)-4-(difluoromethoxy)-1-((4-phenoxybenzoyl)glycyl)pyrrolidine-2-carboxamide (Compound 213)

To a solution of (2S,4R)-N-((R)-1-(4-(N-acetoxycarbamimidoyl)thiophen-2-yl)-2-methoxyethyl)-4-(difluoromethoxy)-1-((4-phenoxybenzoyl)glycyl)pyrrolidine-2-carboxamide (20 mg, 0.03 mmol) in MeOH (3 mL) was added Pd/C (5 mg) at 0° C. The mixture was degassed under N₂ atmosphere for three times and stirred under a H₂ balloon at 25° C. for 1.5 hours. The mixture was filtered and the filtrated was concentrated to dryness. The residue was purified by prep-HPLC to Compound 213 (5.0 mg, yield 27.8%) as a white solid. ¹H NMR (400 MHz, CD₃OD) δ 8.26-8.22 (m, 1H), 7.87-7.82 (m, 2H), 7.58 (d, J=4.8 Hz, 1H), 7.42 (t, J=8.0 Hz, 2H), 7.21 (t, J=7.4 Hz, 1H), 7.06 (d, J=7.6 Hz, 2H), 7.01 (d, J=8.8 Hz, 2H), 6.71-6.27 (m, 1H), 5.48-5.33 (m, 1H), 5.07-4.99 (m, 1H), 4.77-4.61 (m, 1H), 4.33-4.20 (m, 1H), 4.16-4.07 (m, 1H), 3.98-3.92 (m, 1H), 3.89-3.85 (m, 1H), 3.73-3.64 (m, 2H), 3.44 (s, 3H), 2.69-2.44 (m, 1H), 2.42-2.22 (m, 1H). LC/MS (ESI) m/z: 616 (M+H)⁺.

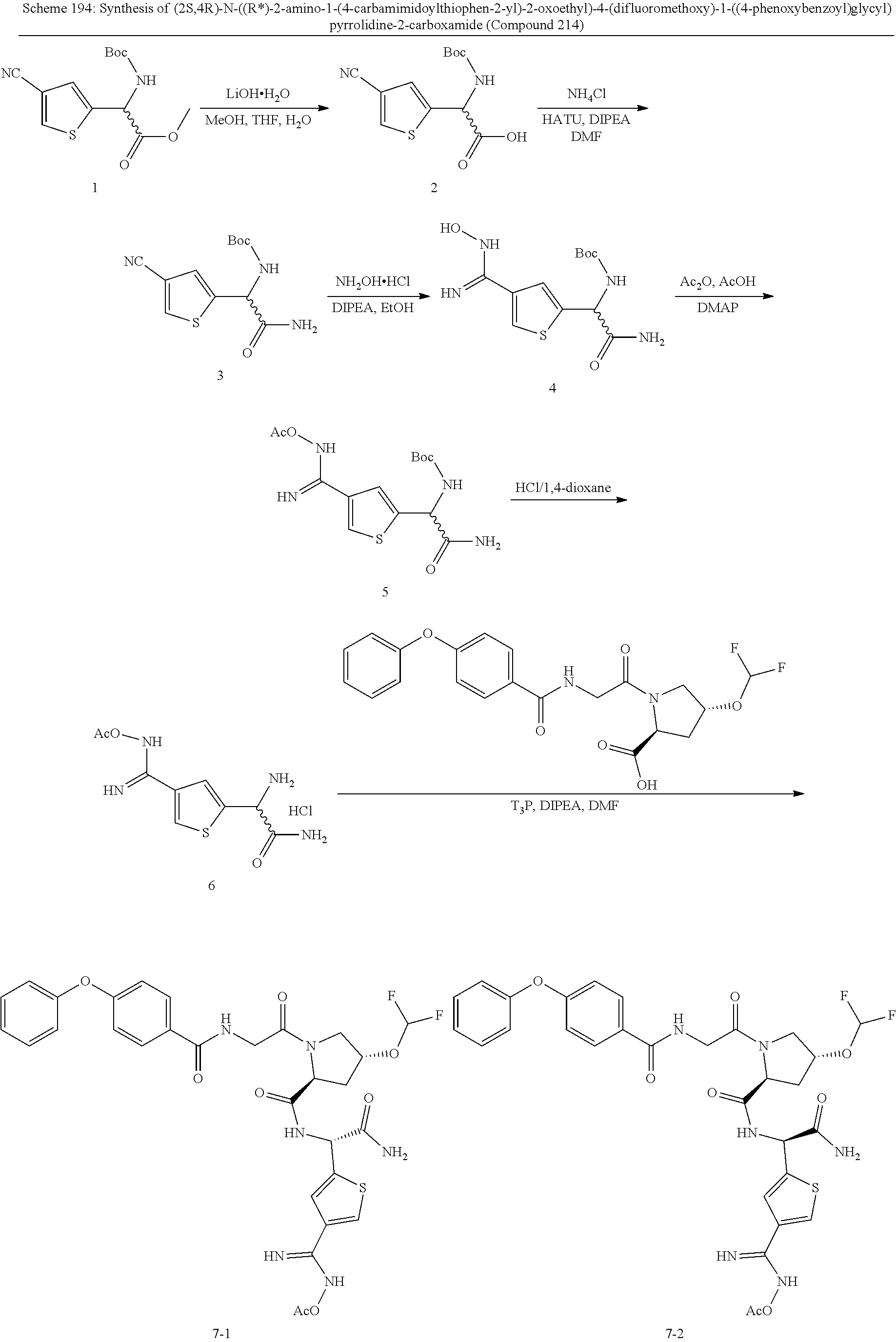

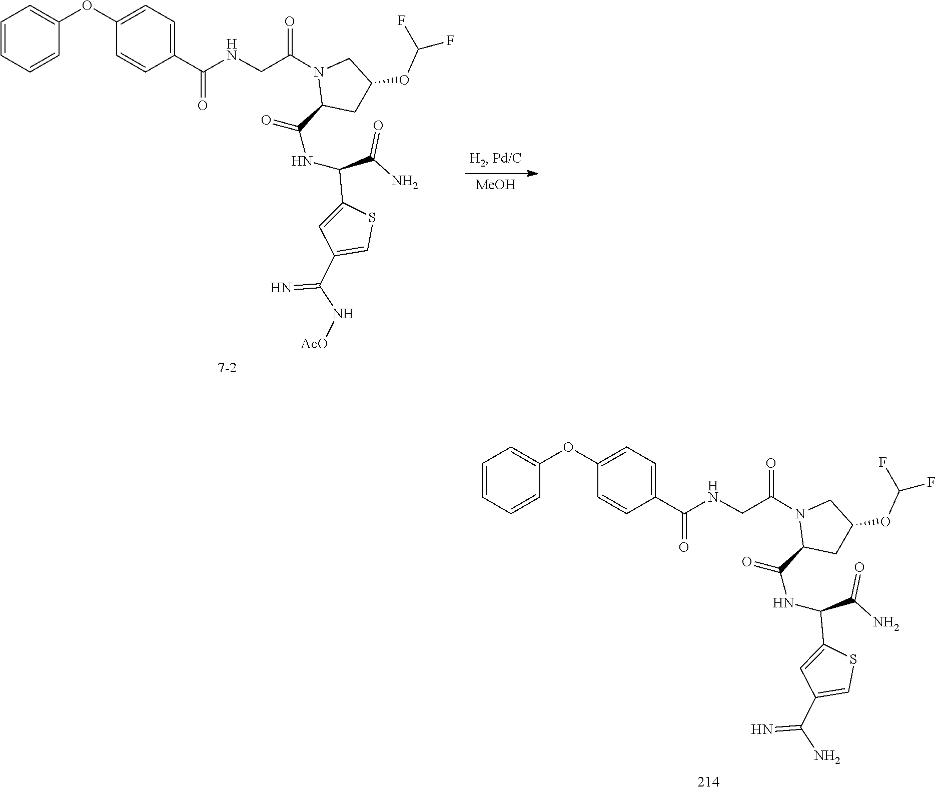 [SMILES: "MeOH" ; CC(=O)ONC(=N)c1csc([C@@H](NC(=O)[C@@H]2C[C@@H](OC(F)F)CN2C(=O)CNC(=O)c2ccc(Oc3ccccc3)cc2)C(N)=O)c1 ; "H2, Pd/C" ; N=C(N)c1csc([C@@H](NC(=O)[C@@H]2C[C@@H](OC(F)F)CN2C(=O)CNC(=O)c2ccc(Oc3ccccc3)cc2)C(N)=O)c1]

### Step 1: 2-((tert-butoxycarbonyl)amino)-2-(4-cyanothiophen-2-yl)acetic acid (2)

To a solution of methyl 2-((tert-butoxycarbonyl)amino)-2-(4-cyanothiophen-2-yl)acetate (100 mg, 0.34 mmol) in MeOH (2 mL), THE (1 mL) and water (1 mL) was added LiOH·H₂O (14 mg, 1.02 mmol) at 0° C. and the mixture was stirred under N₂ atmosphere at room temperature for 3 hours. The mixture was acidified with 1N aq.HCl solution to pH-3 and extracted with EtOAc twice. The combined organic layers were washed with brine, dried over anhydrous Na₂SO₄, filtered and concentrated to dryness under reduced pressure to give 2-((tert-butoxycarbonyl)amino)-2-(4-cyanothiophen-2-yl)acetic acid (90 mg, yield 94.1%) as white solid, which was used directly in next step without further purification. LC/MS (ESI) (m/z): 283 (M+H)⁺.

### Step 2: tert-butyl (2-amino-1-(4-cyanothiophen-2-yl)-2-oxoethyl)carbamate (3)

To a mixture of 2-((tert-butoxycarbonyl)amino)-2-(4-cyanothiophen-2-yl)acetic acid (90 mg, 0.32 mmol) and NH₄Cl (21 mg, 0.38 mmol) in DMF (2 mL) was added DIPEA (0.4 mL, 1.92 mmol) and HATU (365 mg, 0.96 mmol) under N₂ atmosphere at 0° C., and the mixture was stirred at room temperature for 2 hours. The mixture was diluted with EtOAc, washed with water and brine, dried over anhydrous Na₂SO₄, filtered and concentrated to dryness. The residue was purified by flash chromatography on silica gel (PE:EtOAc=1:1) to afford tert-butyl (2-amino-1-(4-cyanothiophen-2-yl)-2-oxoethyl)carbamate (85 mg, yield 95.1%) as colorless oil. LC/MS (ESI) m/z: 282 (M+H)⁺.

### Step 3: tert-butyl (2-amino-1-(4-(N-hydroxycarbamimidoyl)thiophen-2-yl)-2-oxoethyl)carbamate (4)

To a mixture of tert-butyl (2-amino-1-(4-cyanothiophen-2-yl)-2-oxoethyl)carbamate (85 mg, 0.30 mmol) in EtOH (2 mL) was added DIPEA (0.15 mL, 0.75 mmol) and NH₂OH·HCl (49 mg, 0.9 mmol) and the mixture was stirred at 25° C. for 16 hours. The mixture was diluted with EtOAc, washed with water and brine, dried over anhydrous Na₂SO₄, filtered and concentrated to dryness. The residue was purified by prep-TLC (DCM:MeOH=10:1) to give tert-butyl (2-amino-1-(4-(N-hydroxycarbamimidoyl)thiophen-2-yl)-2-oxoethyl)carbamate (90 mg, yield 95%) as colorless oil. LC/MS (ESI) m/z: 315 (M+H)⁺.

### Step 4: tert-butyl (1-(4-(N-acetoxycarbamimidoyl)thiophen-2-yl)-2-amino-2-oxoethyl)carbamate (5)

To a mixture of tert-butyl (2-amino-1-(4-(N-hydroxycarbamimidoyl)thiophen-2-yl)-2-oxoethyl)carbamate (90 mg, 0.29 mmol) in AcOH (2 mL) was added Ac₂O (2 mL) and DMAP (5 mg) and the mixture was stirred at 25° C. for 16 hours. The mixture was diluted with EtOAc, washed with water and brine, dried over anhydrous Na₂SO₄, filtered and concentrated to dryness under reduced pressure. The residue was purified by flash chromatography on silica gel (DCM:MeOH=10:1) to afford tert-butyl (1-(4-(N-acetoxycarbamimidoyl)thiophen-2-yl)-2-amino-2-oxoethyl)carbamate (45 mg, yield 44.1%) as colorless oil. LC/MS (ESI) m/z: 357(M+H)⁺.

### Step 5: 2-(4-(N-acetoxycarbamimidoyl)thiophen-2-yl)-2-aminoacetamide hydrochloride (6)

A mixture of tert-butyl (1-(4-(N-acetoxycarbamimidoyl)thiophen-2-yl)-2-amino-2-oxoethyl)carbamate (45 mg, 0.13 mmol) in HCl/1,4-dioxane (3 mL) was stirred at room temperature for 2 hours. The reaction mixture was concentrated to dryness under reduced pressure, co-evaporated with DCM and dried under vacuum to give 2-(4-(N-acetoxycarbamimidoyl)thiophen-2-yl)-2-aminoacetamide hydrochloride (40 mg, yield 100%) as colorless oil, which was used directly in the next reaction without further purification. LC/MS (ESI) m/z: 257 (M+H)⁺.

### Step 6: (2S,4R)-N-((R*)-1-(4-(N-acetoxycarbamimidoyl)thiophen-2-yl)-2-amino-2-oxoethyl)-4-(difluoromethoxy)-1-((4-phenoxybenzoyl)glycyl)pyrrolidine-2-carboxamide (7-2) and (2S,4R)-N-((S*)-1-(4-(N-acetoxycarbamimidoyl)thiophen-2-yl)-2-amino-2-oxoethyl)-4-(difluoromethoxy)-1-((4-phenoxybenzoyl)glycyl)pyrrolidine-2-carboxamide (7-1)

To a mixture of 2-(4-(N-acetoxycarbamimidoyl)thiophen-2-yl)-2-aminoacetamide hydrochloride (40 mg, 0.13 mmol) and (2S,4R)-4-(difluoromethoxy)-1-((4-phenoxybenzoyl)glycyl)pyrrolidine-2-carboxylic acid (61 mg, 0.14 mmol) in DMF (2 mL) was added DIPEA (0.10 mL, 0.59 mmol) and T₃P (89 mg, 0.14 mmol, 50% wt in EtOAc) at 0° C. under N₂ atmosphere and the mixture was stirred at 25° C. for 2 hours. The mixture was quenched with water and extracted with EtOAc twice. The combined organic layers were washed with brine, dried over anhydrous Na₂SO₄, filtered and concentrated to dryness under reduced pressure. The residue was purified by prep-TLC (DCM:MeOH=10:1) to give (2S,4R)-N-((R*)-1-(4-(N-acetoxycarbamimidoyl)thiophen-2-yl)-2-amino-2-oxoethyl)-4-(difluoromethoxy)-1-((4-phenoxybenzoyl) glycyl)pyrrolidine-2-carboxamide (15 mg, yield 11.5%); LC/MS (ESI) m/z: 673 (M+H)⁺ and (2S,4R)-N-((S*)-1-(4-(N-acetoxycarbamimidoyl)thiophen-2-yl)-2-amino-2-oxoethyl)-4-(difluoromethoxy)-1-((4-phenoxybenzoyl)glycyl)pyrrolidine-2-carboxamide (25 mg, yield 19.1%) as colorless oil. LC/MS (ESI) m/z: 673 (M+H)⁺.

Step 7: (2S,4R)-N-((S*)-2-amino-1-(4-carbamimidoylthiophen-2-yl)-2-oxoethyl)-4-(difluoromethoxy)-1-((4-phenoxybenzoyl)glycyl)pyrrolidine-2-carboxamide (Compound 214) To a mixture of (2S,4R)-N-((R*)-1-(4-(N-acetoxycarbamimidoyl)thiophen-2-yl)-2-amino-2-oxoethyl)-4-(difluoromethoxy)-1-((4-phenoxybenzoyl)glycyl)pyrrolidine-2-carboxamide (15 mg, 0.02 mmol) in MeOH (2 mL) was added Pd/C (5 mg, 10% wt.), the mixture was degassed under N₂ atmosphere for three times and stirred under a H₂ balloon at 25° C. for 2 hours. The mixture was filtered and the filtrate was concentrated under reduced pressure to dryness. The residue was purified by prep-HPLC to give (2S,4R)-N-((R*)-2-amino-1-(4-carbamimidoylthiophen-2-yl)-2-oxoethyl)-4-(difluoromethoxy)-1-((4-phenoxybenzoyl) glycyl)pyrrolidine-2-carboxamide (4.0 mg, yield 29.2%) as white solid. ¹HNMR (400 MHz, CD₃OD) δ 8.46 (s, 1H), 8.24 (d, J=1.6 Hz, 1H), 7.80-7.76 (m, 2H), 7.56 (t, J=23.4 Hz, 1H), 7.36 (t, J=7.9 Hz, 2H), 7.15 (t, J=7.4 Hz, 1H), 7.03-6.99 (m, 2H), 6.95 (dd, J=8.8, 1.6 Hz, 2H), 6.46 (t, J=74.4 Hz, 1H), 5.76 (dd, J=24.6, 17.6 Hz, 1H), 4.97 (d, J=3.6 Hz, 1H), 4.64-4.52 (m, 1H), 4.15 (m, J=16.7, 9.6 Hz, 2H), 3.95-3.88 (m, 1H), 3.83 (t, J=8.3 Hz, 1H), 2.49-2.35 (m, 1H), 2.27-2.16 (m, 1H). LC/MS (ESI) m/z: 615 (M+H)⁺.

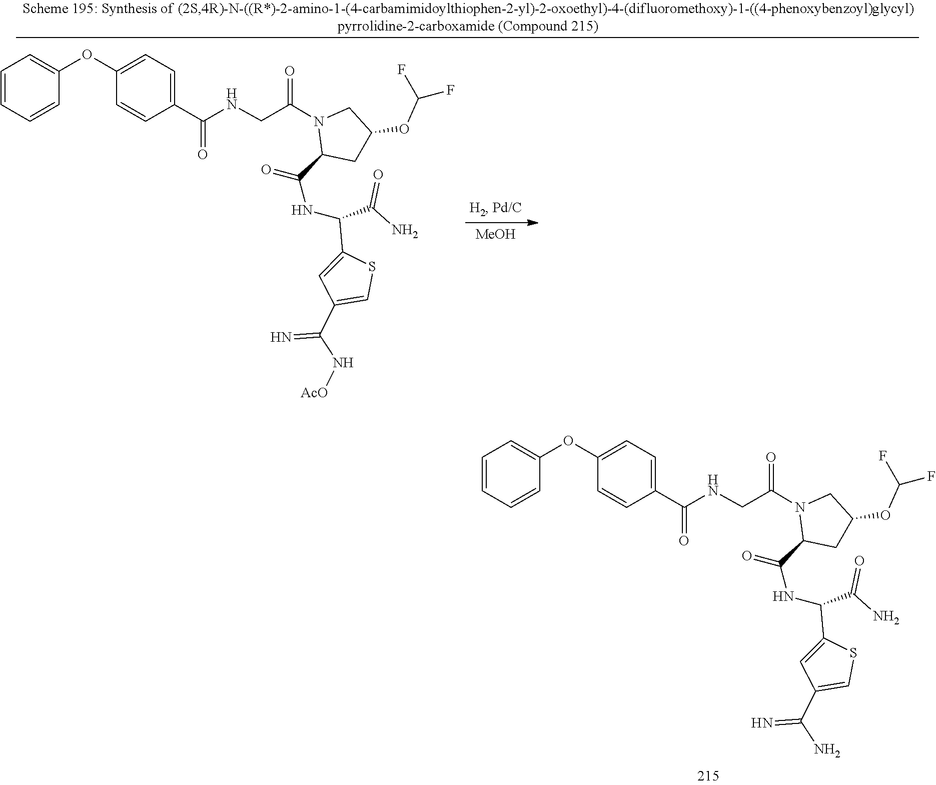 [SMILES: "MeOH" ; CC(=O)ONC(=N)c1csc([C@H](NC(=O)[C@@H]2C[C@@H](OC(F)F)CN2C(=O)CNC(=O)c2ccc(Oc3ccccc3)cc2)C(N)=O)c1 ; "H2, Pd/C" ; N=C(N)c1csc([C@H](NC(=O)[C@@H]2C[C@@H](OC(F)F)CN2C(=O)CNC(=O)c2ccc(Oc3ccccc3)cc2)C(N)=O)c1]

To a mixture of (2S,4R)-N-((S*)-1-(4-(N-acetoxycarbamimidoyl)thiophen-2-yl)-2-amino-2-oxoethyl)-4-(difluoromethoxy)-1-((4-phenoxybenzoyl)glycyl)pyrrolidine-2-carboxamide (25 mg, 0.037 mmol) in MeOH (2 mL) was added Pd/C (5 mg, 10% wt.), the mixture was degassed under N₂ atmosphere for three times and stirred under a H₂ balloon at 25° C. for 2 hours. The mixture was filtered and the filtrate was concentrated to dryness under reduced pressure. The residue was purified by prep-HPLC to give Compound 215 (5.0 mg, yield 21.9%) as white solid. ¹HNMR (400 MHz, CD₃OD) δ 8.51 (s, 1H), 8.29 (d, J=1.4 Hz, 1H), 7.83 (dd, J=8.8, 3.0 Hz, 2H), 7.62 (dd, J=25.8, 20.3 Hz, 1H), 7.41 (t, J=7.9 Hz, 2H), 7.20 (t, J=7.4 Hz, 1H), 7.06 (d, J=7.7 Hz, 2H), 7.00 (d, J=8.8 Hz, 2H), 6.51 (t, J=74.4 Hz, 1H), 5.81 (dd, J=25.1, 17.8 Hz, 1H), 5.03 (s, 1H), 4.69-4.59 (m, 1H), 4.29-4.12 (m, 2H), 4.02-3.94 (m, 1H), 3.88 (d, J=10.8 Hz, 1H), 2.54-2.42 (m, 1H), 2.32-2.23 (m, 1H). LC/MS (ESI) m/z: 615 (M+H)⁺.

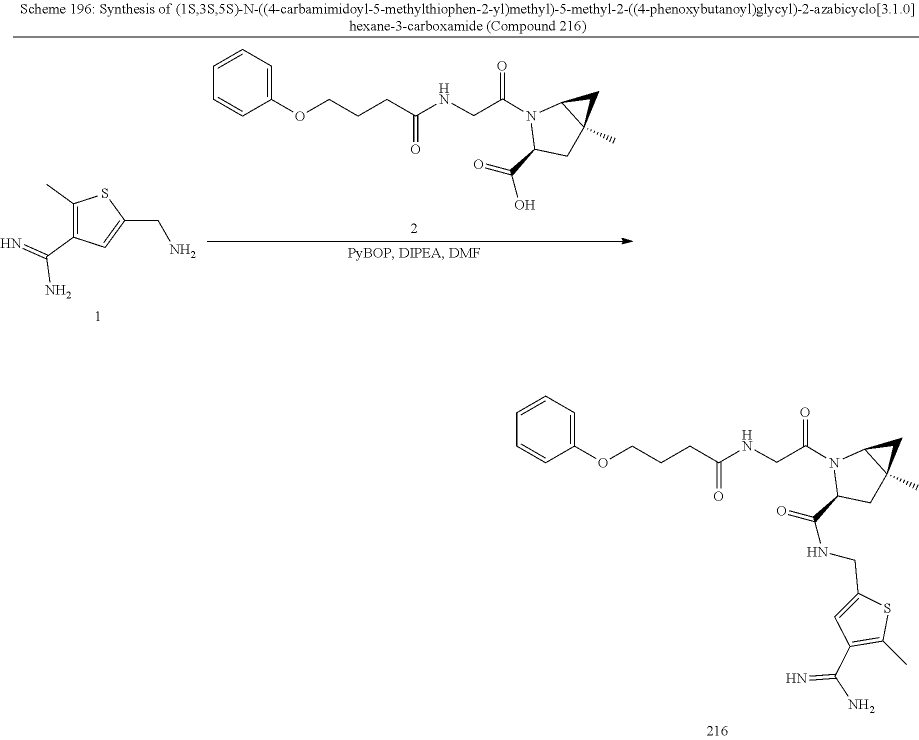 [SMILES: Cc1sc(CN)cc1C(=N)N ; C[C@@]12C[C@@H]1N(C(=O)CNC(=O)CCCOc1ccccc1)[C@H](C(=O)O)C2 ; Cc1sc(CNC(=O)[C@@H]2C[C@]3(C)C[C@@H]3N2C(=O)CNC(=O)CCCOc2ccccc2)cc1C(=N)N]

### Step 1: (1S,3S,5S)-N-((4-carbamimidoyl-5-methylthiophen-2-yl)methyl)-5-methyl-2-((4-phenoxybutanoyl)glycyl)-2-azabicyclo[3.1.0]hexane-3-carboxamide (Compound 216)

To a mixture of (1 S,3S,5S)-5-methyl-2-((4-phenoxybutanoyl)glycyl)-2-azabicyclo[3.1.0]hexane-3-carboxylic acid (43 mg, 0.12 mmol) and 5-(aminomethyl)-2-methylthiophene-3-carboximidamide (20 mg, 0.12 mmol) in DMF (2 mL) was added DIPEA (0.1 mL, 0.60 mmol) and PyBOP (94 mg, 0.18 mmol) at 0° C. under N₂ atmosphere and the mixture was stirred at room temperature for 1 hour. The mixture was diluted with CHCl₃/i-PrOH (3/1, v/v) twice, washed with water and brine, dried over anhydrous Na₂SO₄, filtered and concentrated under reduced pressure to dryness. The residue was purified by prep-TLC (DCM:MeOH=10:1) and prep-HPLC to afford Compound 216 (2.0 mg, yield 3.3%) as a white solid. ¹HNMR (400 MHz, CD₃OD) δ 7.27-7.22 (m, 2H), 7.03 (s, 1H), 6.92-6.88 (m, 3H), 4.77 (dd, J=11.2, 11.2 Hz, 1H), 4.47-4.40 (m, 2H), 4.22-4.10 (m, 2H), 4.01 (t, J=6.2 Hz, 2H), 3.35-3.32 (m, 1H), 2.58 (s, 3H), 2.50-2.45 (m, 2H), 2.41-2.33 (m, 1H), 2.16-2.11 (m, 1H), 2.10-2.04 (m, 2H), 1.28 (s, 3H), 1.11 (dd, J=5.6, 5.6 Hz, 1H), 0.83-0.77 (m, 1H). LC/MS (ESI) (m/z): 512 (M+H)⁺.

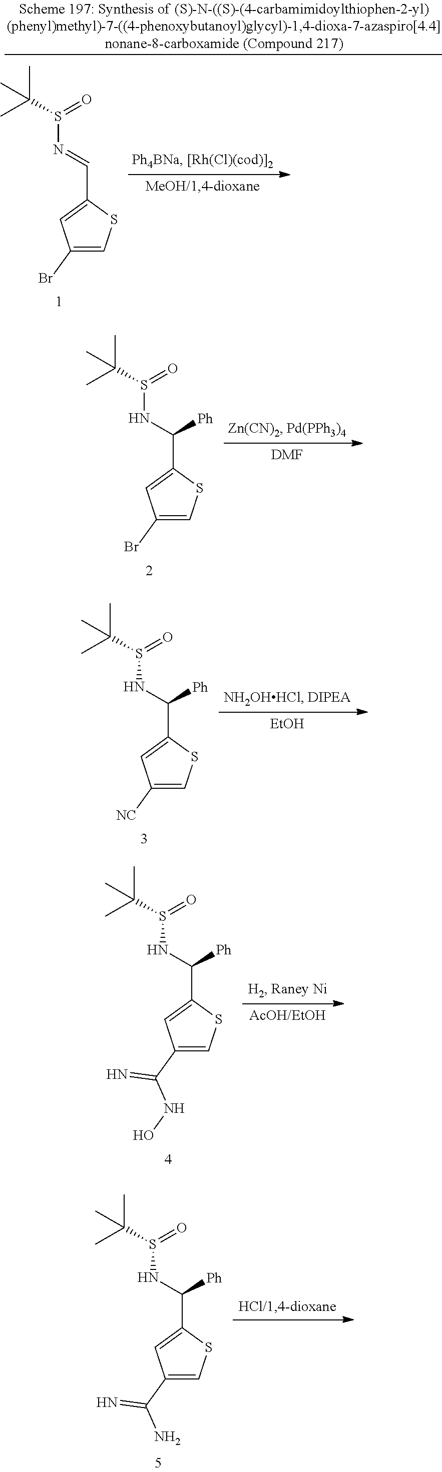

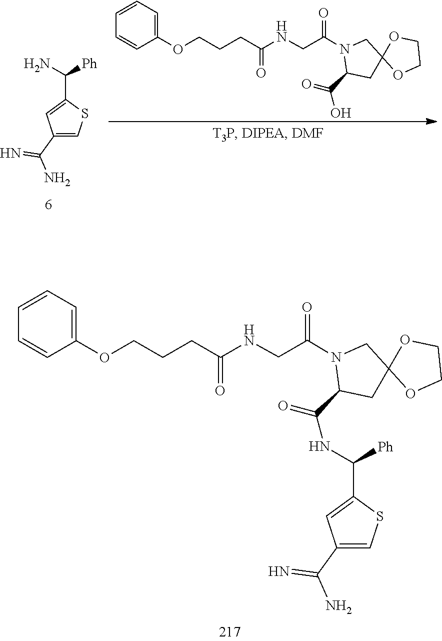

### Step 1: (S)-N-((S)-(4-bromothiophen-2-yl)(phenyl)methyl)-2-methylpropane-2-sulfinamide (2)

To a solution of (S,E)-N-((4-bromothiophen-2-yl)methylene)-2-methylpropane-2-sulfinamide (1 g, 3.40 mmol) and MeOH (218 mg, 6.80 mmol) in 1,4-dioxane (10 mL) was added Ph₄BNa (1.40 g, 4.08 mmol) and [Rh(Cl)(cod)]2 (34 mg, 0.068 mmol). The reaction mixture was stirred at 65° C. under N₂ atmosphere overnight. The mixture was diluted with EtOAc, washed with water and brine, dried over anhydrous Na₂SO₄, filtered and concentrated under reduced pressure to dryness. The residue was purified by flash chromatography on silica gel (PE:EtOAc=5:1) to give (S)-N-((S)-(4-bromothiophen-2-yl)(phenyl)methyl)-2-methylpropane-2-sulfinamide (1.2 g, yield 94.9%) as white solid. LC/MS (ESI) m/z:372 (M+H)⁺.

### Step 2: (S)-N-((S)-(4-cyanothiophen-2-yl)(phenyl)methyl)-2-methylpropane-2-sulfinamide (3)

To a solution of (S)-N-((S)-(4-bromothiophen-2-yl)(phenyl) methyl)-2-methyl propane-2-sulfinamide (400 mg, 1.07 mmol) in NMP (10 mL) was added Zn(CN)₂ (252 mg, 2.15 mmol) and Pd(PPh₃)₄ (186 mg, 0.16 mmol). The mixture was degassed under N₂ atmosphere for three times and stirred at 160° C. for 1 hour. The mixture was diluted with EtOAc, washed with water and brine, dried over anhydrous Na₂SO₄, filtered and concentrated under reduced pressure to dryness. The residue was purified by flash chromatography on silica gel (PE:EtOAc=5:1) to give (S)-N-((S)-(4-cyanothiophen-2-yl)(phenyl)methyl)-2-methylpropane-2-sulfinamide (120 mg, yield 35.1%) as white solid. LC/MS (ESI) m/z: 319(M+H)⁺.

### Step 3: 5-((S)-(((S)-tert-butylsulfinyl)amino)(phenyl)methyl)-N-hydroxythiophene-3-carboximidamide (4)

To a solution of N-((R)-(4-cyanothiophen-2-yl)(phenyl)methyl)-2-methylpropane-2-sulfinamide (120 mg, 0.38 mmol) in EtOH (5 mL) was added NH₂OH hydrochloride (65 mg, 0.94 mmol) and DIPEA (146 mg, 1.13 mmol) and the reaction mixture was stirred at room temperature for 3 hours. The mixture was diluted with EtOAc, washed with water and brine, dried over anhydrous Na₂SO₄, filtered and concentrated under reduced pressure to dryness. The residue was purified by flash chromatography on silica gel (DCM:MeOH=10:1) to give 5-((S)-(((S)-tert-butylsulfinyl)amino)(phenyl)methyl)-N-hydroxythiophene-3-carboximidamide (100 mg, yield 75.5%) as white solid. LC/MS (ESI) m/z: 352 (M+H)⁺.

### Step 4: 5-((S)-(((S)-tert-butylsulfinyl)amino)(phenyl)methyl)thiophene-3-carboximidamide(5)

To a solution of 5-((S)-(((S)-tert-butylsulfinyl)amino)(phenyl)methyl)-N-hydroxythiophene-3-carboximidamide (100 mg, 0.28 mmol) in MeOH (2 mL) was added Raney Ni (20 mg) and AcOH (0.1 mL) under N₂ atmosphere and the reaction mixture was stirred under a H₂ balloon for 3 hours. The mixture was filtered and the filtrate was concentrated under reduced pressure to give 5-((S)-(((S)-tert-butylsulfinyl)amino)(phenyl)methyl)thiophene-3-carboximidamide (95 mg, yield 99.5%) as white solid. LC/MS (ESI) m/z: 336 (M+H)⁺.

### Step 5: (S)-5-(amino(phenyl)methyl)thiophene-3-carboximidamide hydrochloride(6)

To a solution of 5-((S)-(((S)-tert-butylsulfinyl)amino)(phenyl)methyl)thiophene-3-carboximidamide (95 mg, 0.28 mmol) in 1,4-dioxane (2 mL) was added HCl/1,4-dioxane (1 mL, 4M) under N₂ atmosphere and the reaction mixture was stirred at room temperature for 2 hours. The mixture was concentrated to dryness under reduced pressure to give (S)-5-(amino(phenyl)methyl)thiophene-3-carboximidamide hydrochloride (85 mg, yield 100%) as white solid. LC/MS (ESI) m/z: 232 (M+H)⁺.

### Step 6: (S)-N-((S)-(4-carbamimidoylthiophen-2-yl)(phenyl)methyl)-7-((4-phenoxybutanoyl)glycyl)-1,4-dioxa-7-azaspiro[4.4]nonane-8-carboxamide (Compound 217)

To a mixture of (S)-5-(amino(phenyl)methyl)thiophene-3-carboximidamide hydrochloride (85 mg, 0.28 mmol) and (S)-7-((4-phenoxybutanoyl)glycyl)-1,4-dioxa-7-azaspiro[4.4]nonane-8-carboxylic acid (110 mg, 0.28 mmol) in DMF (5 mL) was added DIPEA (218 mg, 1.69 mmol) and T₃P (268 mg, 0.42 mmol, 50% wt. in EtOAc) under N₂ atmosphere and the reaction mixture was stirred at room temperature for 2 hours. The mixture was diluted with EtOAc, washed with saturated aq.NaHCO₃ solution and brine, dried over anhydrous Na₂SO₄, filtered and concentrated under reduced pressure to dryness. The residue was purified by prep-TLC (DCM:MeOH=5:1) and further purified by prep-HPLC to give Compound 217 (10 mg, yield 5.9%) as a white solid. ¹H NMR (400 MHz, CD₃OD) δ 8.49 (s, 1H), 8.28-8.22 (m, 1H), 7.47 (dd, J=22.2, 5.9 Hz, 2H), 7.41-7.31 (m, 4H), 7.25-7.20 (m, 2H), 6.89 (dd, J=7.5, 5.7 Hz, 3H), 6.51-6.35 (m, 1H), 4.66 (ddd, J=14.5, 9.3, 4.5 Hz, 1H), 4.06-3.55 (m, 11H), 2.48-2.35 (m, 3H), 2.31-2.14 (m, 1H), 2.07-1.96 (m, 2H). LC/MS (ESI) m/z: 606 (M+H)⁺.

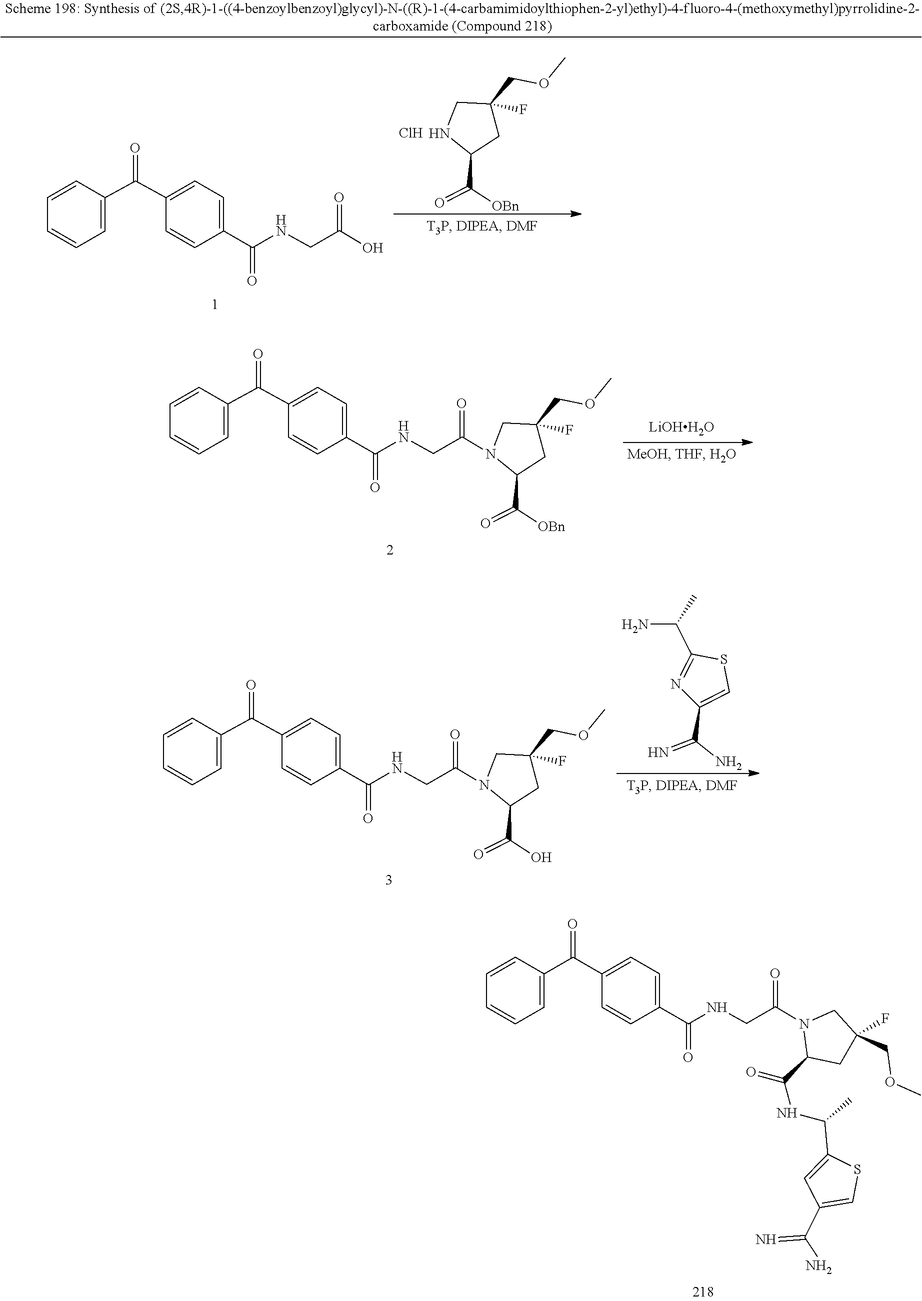

### Step 1: benzyl (2S,4R)-1-((4-benzoylbenzoyl)glycyl)-4-fluoro-4-(methoxymethyl)pyrrolidine-2-carboxylate (2)

To a mixture of (4-benzoylbenzoyl)glycine (50 mg, 0.18 mmol) and benzyl (2S,4R)-4-fluoro-4-(methoxymethyl)pyrrolidine-2-carboxylate hydrochloride (64 mg, 0.22 mmol) in DMF (2 mL) was added DIPEA (113 mg, 0.87 mmol) and T₃P (167 mg, 0.26 mmol, 50% wt. in EtOAc) at 0° C. under N₂ atmosphere and the mixture was stirred at room temperature for 2 hours. The mixture was diluted with EtOAc and washed with saturated aq.NaHCO₃ solution. The aqueous layer was extracted with EtOAc twice. The combined organic layers were washed with brine, dried over anhydrous Na₂SO₄, filtered and concentrated under reduced pressure to dryness. The residue was purified by flash chromatography on silica gel (PE:EtOAc=1:1) to give benzyl (2S,4R)-1-((4-benzoylbenzoyl)glycyl)-4-fluoro-4-(methoxymethyl)pyrrolidine-2-carboxylate (85 mg, yield 91.4%) as colorless oil. LC/MS (ESI) m/z: 533 (M+H)⁺.

### Step 2: (2S,4R)-1-((4-benzoylbenzoyl)glycyl)-4-fluoro-4-(methoxymethyl)pyrrolidine-2-carboxylic acid (3)

To a solution of benzyl (2S,4R)-1-((4-benzoylbenzoyl)glycyl)-4-fluoro-4-(methoxymethyl)pyrrolidine-2-carboxylate (80 mg, 0.15 mmol) in MeOH (2 mL) and THE (1 mL) was added a solution of LiOH·H₂O (19 mg, 0.45 mmol) in water (1 mL) at 0° C., and the mixture was stirred at room temperature for 2 hours. The mixture was diluted with EtOAc and washed with water twice. The aqueous layer was acidified with 0.5 M aq.HCl to pH-3 and extracted with EtOAc twice. The combined organic layers were washed with brine, dried over anhydrous Na₂SO₄, filtered and concentrated to dryness to give (2S,4R)-1-((4-benzoylbenzoyl)glycyl)-4-fluoro-4-(methoxymethyl)pyrrolidine-2-carboxylic acid (60 mg, yield 86.7%) as colorless oil. LC/MS (ESI) (m/z): 443 (M+H)⁺.

### Step 5: (2S,4R)-1-((4-benzoylbenzoyl)glycyl)-N-((R)-1-(4-carbamimidoylthiophen-2-yl)ethyl)-4-fluoro-4-(methoxymethyl)pyrrolidine-2-carboxamide (Compound 218)

To a mixture of (2S,4R)-1-((4-benzoylbenzoyl)glycyl)-4-fluoro-4-(methoxymethyl)pyrrolidine-2-carboxylic acid (60 mg, 0.14 mmol) and (R)-5-(1-aminoethyl)thiophene-3-carboximidamide (35 mg, 0.21 mmol) in DMF (2 mL) was added DIPEA (90 mg, 0.69 mmol) and T₃P (106 mg, 0.17 mmol, 50% in EtOAc) at 0° C. and the mixture was stirred at room temperature for 2 hours. The mixture was diluted with water and extracted with EtOAc twice. The combined organic layers were washed with brine, dried over anhydrous Na₂SO₄, filtered and concentrated under reduced pressure to dryness. The residue was purified by prep-TLC (DCM:MeOH=10:1) and further purified by prep-HPLC to give Compound 218 (10.5 mg, yield 12.5%) as a white solid. ¹H NMR (400 MHz, CD₃OD) δ 8.57 (s, 1H), 8.26 (t, J=7.4 Hz, 1H), 8.02 (t, J=8.1 Hz, 2H), 7.88 (d, J=8.4 Hz, 2H), 7.85-7.81 (m, 2H), 7.70 (dd, J=10.6, 4.3 Hz, 1H), 7.61-7.56 (m, 3H), 5.33 (dq, J=14.0, 7.1 Hz, 1H), 4.72-4.61 (m, 1H), 4.37 (d, J=16.7 Hz, 1H), 4.19 (d, J=16.6 Hz, 1H), 4.09 (dd, J=19.7, 12.4 Hz, 1H), 4.00-3.88 (m, 1H), 3.77 (t, J=7.6 Hz, 1H), 3.72 (d, J=6.9 Hz, 1H), 3.46 (d, J=6.4 Hz, 3H), 2.58 (td, J=14.8, 7.9 Hz, 1H), 2.22 (m, J=36.8, 14.5, 9.6 Hz, 1H), 1.65 (dd, J=32.4, 7.0 Hz, 3H). LC/MS (ESI) m/z: 595

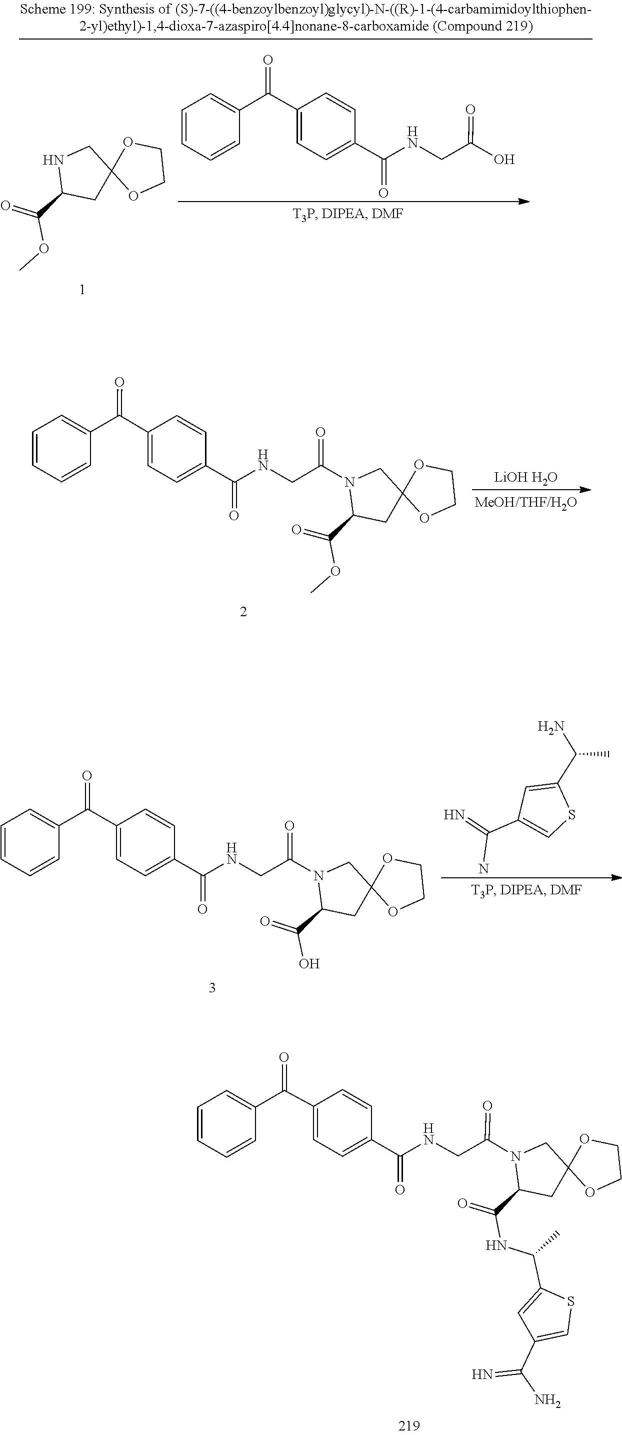

### Step 1: methyl (S)-7-((4-benzoylbenzoyl)glycyl)-1,4-dioxa-7-azaspiro[4.4]nonane-8-carboxylate (2)

At 0° C., to a mixture of methyl (S)-1,4-dioxa-7-azaspiro[4.4]nonane-8-carboxylate (40 mg, 0.21 mmol) and (4-benzoylbenzoyl)glycine (60 mg, 0.21 mmol) in DMF (2 mL) was added T₃P (479 mg, 0.63 mmol, 50% wt. in EtOAc) and DIPEA (0.2 mL, 1.26 mmol) under N₂ atmosphere and the mixture was stirred at 30° C. overnight. The mixture was diluted with EtOAc and washed with saturated aq.NaHCO₃ solution. The aqueous layer was extracted with EtOAc and the combined organic layers were washed with brine, dried over anhydrous Na₂SO₄, filtered and concentrated under reduced pressure to dryness. The residue was purified by flash chromatography on silica gel (DCM:MeOH=20:1) to afford methyl (S)-7-((4-benzoylbenzoyl)glycyl)-1,4-dioxa-7-azaspiro[4.4]nonane-8-carboxylate (72 mg, yield 76.6%) as yellow oil. LC/MS (ESI) m/z: 453 (M+H)⁺.

### Step 2: (S)-7-((4-benzoylbenzoyl)glycyl)-1,4-dioxa-7-azaspiro[4.4]nonane-8-carboxylic acid (3)

To a solution of methyl (S)-7-((4-benzoylbenzoyl)glycyl)-1,4-dioxa-7-azaspiro[4.4]nonane-8-carboxylate (72 mg, 0.16 mmol) in THF/MeOH/H₂O (4 mL, 2/1/1) was added LiOH·H₂O (7 mg, 0.16 mmol) and the reaction mixture was stirred at room temperature for 16 hours. The mixture was concentrated to dryness, diluted with water and washed with EtOAc twice. The aqueous layer was acidified with 1N aq.HCl to pH-4 and extracted with EtOAc twice. The combined organic layers were washed with brine, dried over anhydrous Na₂SO₄, filtered and concentrated to dryness to afford (S)-7-((4-benzoylbenzoyl)glycyl)-1,4-dioxa-7-azaspiro[4.4]nonane-8-carboxylic acid (50 mg, yield 72.5%) as yellow oil. LC/MS (ESI) m/z: 439 (M+H)⁺.

### Step 3: (S)-7-((4-benzoylbenzoyl)glycyl)-N-((R)-1-(4-carbamimidoylthiophen-2-yl)ethyl)-1,4-dioxa-7-azaspiro[4.4]nonane-8-carboxamide (Compound 219)

At 0° C., to a mixture of (R)-7-((4-benzoylbenzoyl)glycyl)-1,4-dioxa-7-azaspiro[4.4]nonane-8-carboxylic acid (60 mg, 0.14 mmol) and (R)-5-(1-aminoethyl)thiophene-3-carboximidamide (35 mg, 0.21 mmol) in DMF (2 mL) was added PyBOP (109 mg, 0.21 mmol) and DIPEA (0.1 mL, 0.42 mmol) under N₂ atmosphere and the mixture was stirred at room temperature for 1 hour. The mixture was diluted with EtOAc, washed with water and brine, dried over anhydrous Na₂SO₄, filtered and concentrated under reduced pressure to dryness. The residue was purified prep-HPLC to afford Compound 219 (5 mg, yield 6.1%) as a white solid. ¹H NMR (400 MHz, CD₃OD) δ 8.24 (dd, J=10.0, 10.0 Hz, 1H), 8.01-7.98 (m, 2H), 7.85 (d, J=8.4 Hz, 2H), 7.81-7.78 (m, 2H), 7.67 (t, J=7.4 Hz, 1H), 7.57-7.51 (m, 3H), 5.39-5.24 (m, 1H), 4.56 (dd, J=8.8, 8.4 Hz, 1H), 4.20 (d, J=4.0 Hz, 1H), 4.04-3.93 (m, 5H), 3.83-3.75 (m, 2H), 2.43 (dd, J=13.2, 13.2 Hz, 1H), 2.21 (dd, J=12.8, 13.2 Hz, 1H), 1.58 (d, J=7.0 Hz, 3H). LC/MS (ESI) m/z: 590 (M+H)⁺.

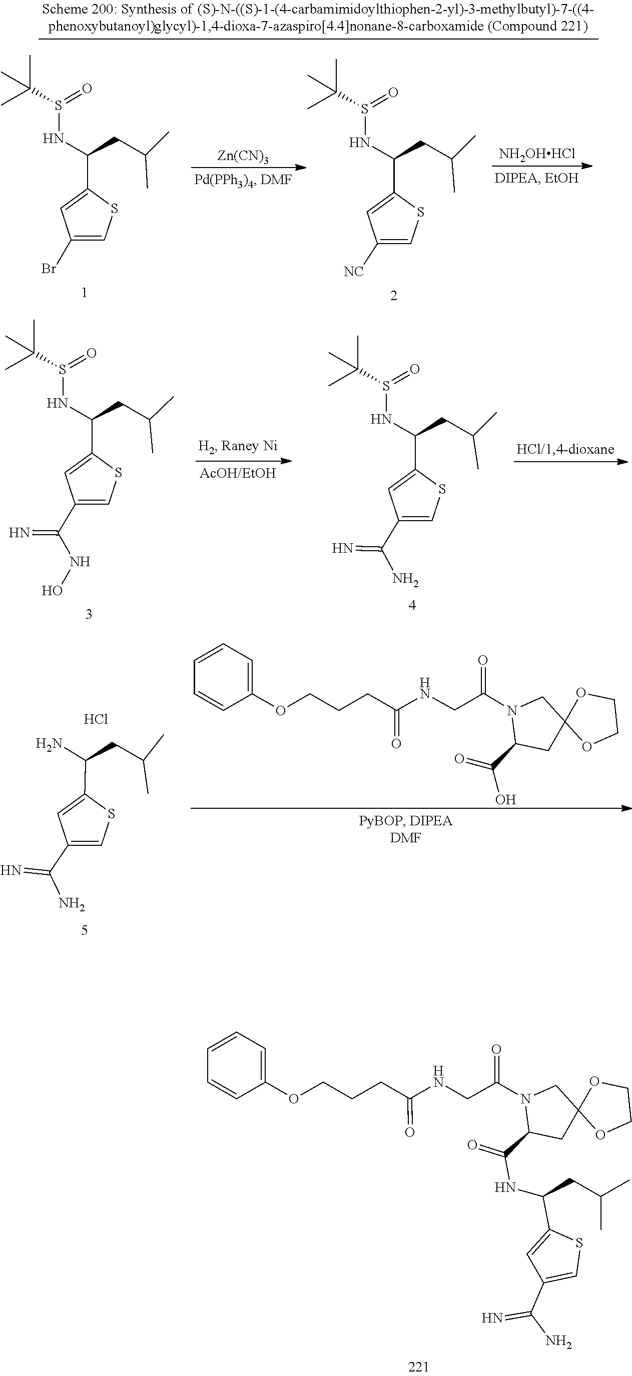

### Step 1: (S)-N-((S)-1-(4-cyanothiophen-2-yl)-3-methylbutyl)-2-methylpropane-2-sulfinamide (2)

To a solution of (S)-N-((S)-1-(4-bromothiophen-2-yl)-3-methylbutyl)-2-methylpropane-2-sulfinamide (150 mg, 0.43 mmol) in DMF (3 mL) was added Zn(CN)₂ (101 mg, 0.86 mmol) and Pd(PPh₃)₄ (75 mg, 0.06 mmol). The reaction mixture was degassed under N₂ atmosphere for three times and stirred at 160° C. for 2 hours. The mixture was diluted with EtOAc, washed with water and brine, dried over anhydrous Na₂SO₄, filtered and concentrated under reduced pressure to dryness. The residue was purified by flash chromatography on silica gel (PE:EtOAc=1:1) to afford (S)-N-((S)-1-(4-cyanothiophen-2-yl)-3-methylbutyl)-2-methylpropane-2-sulfinamide (107 mg, yield 84.3%) as colorless oil. LC/MS (ESI) m/z: 299 (M+H)⁺.

### Step 2: 5-((S)-1-(((S)-tert-butylsulfinyl)amino)-3-methylbutyl)-N-hydroxythiophene-3-carboximidamide (3)

To a solution of (S)-N-((S)-1-(4-cyanothiophen-2-yl)-3-methylbutyl)-2-methylpropane-2-sulfinamide (100 mg, 0.34 mmol) in EtOH (2 mL) was added DIPEA (132 mL, 1.02 mmol) and NH₂OH·HCl (55 mg, 0.85 mmol) and the mixture was stirred at 25° C. for 16 hours. The mixture was diluted with EtOAc, washed with water and brine, dried over anhydrous Na₂SO₄, filtered and concentrated to dryness. The residue was purified by flash chromatography on silica gel (PE:EtOAc=2:1) to give 5-((S)-1-(((S)-tert-butylsulfinyl)amino)-3-methylbutyl)-N-hydroxythiophene-3-carboximidamide (61 mg, yield 55.0%) as green oil. LC/MS (ESI) m/z: 332 (M+H)⁺.

### Step 3: 5-((S)-1-(((S)-tert-butylsulfinyl)amino)-3-methylbutyl)thiophene-3-carboximidamide (4)

To a solution of 5-((S)-1-(((S)-tert-butylsulfinyl)amino)-3-methylbutyl)-N-hydroxythiophene-3-carboximidamide (61 mg, 0.18 mmol) in MeOH (2 mL) and AcOH (0.02 mL) was added Raney Ni (10 mg) at 0° C., and the mixture was degassed under N₂ atmosphere for three times and stirred under a H₂ balloon at room temperature for 16 hours. The mixture was filtered and the filtrate was concentrated under reduced pressure to give 5-((S)-1-(((S)-tert-butylsulfinyl)amino)-3-methylbutyl)thiophene-3-carboximidamide (56 mg, yield 96.2%) as green oil, which was used directly in the next reaction without further purification. LC/MS (ESI) (m/z): 316 (M+H)⁺.

### Step 4: (S)-5-(1-amino-3-methylbutyl)thiophene-3-carboximidamide hydrochloride (5)

A mixture of 5-((S)-1-(((S)-tert-butylsulfinyl)amino)-3-methylbutyl)thiophene-3-carboximidamide (56 mg, 0.18 mmol) in HCl/1,4-dioxane (2 mL) was stirred at room temperature for 1 hour. The reaction mixture was concentrated to dryness under reduced pressure, co-evaporated with DCM and dried under vacuum to give (S)-5-(1-amino-3-methylbutyl)thiophene-3-carboximidamide hydrochloride (30 mg, yield 80.2%) as colorless oil, which was used directly in the next reaction without further purification. LC/MS (ESI) m/z: 212 (M+H)⁺.

### Step 5: (S)-N-((S)-1-(4-carbamimidoylthiophen-2-yl)-3-methylbutyl)-7-((4-phenoxybutanoyl) glycyl)-1,4-dioxa-7-azaspiro[4.4]nonane-8-carboxamide (Compound 221)

To a mixture of (S)-5-(1-amino-3-methylbutyl)thiophene-3-carboximidamide hydrochloride (30 mg, 0.14 mmol) and (S)-7-((4-phenoxybutanoyl)glycyl)-1,4-dioxa-7-azaspiro[4.4]nonane-8-carboxylic acid (44 mg, 0.11 mmol) in DMF (2 mL) was added DIPEA (92 mg, 0.70 mmol) and PyBOP (111 mg, 0.21 mmol) at 0° C. and the mixture was stirred at room temperature for 2 hours. The mixture was diluted with water and extracted with EtOAc twice. The combined organic layers were washed with brine, dried over anhydrous Na₂SO₄, filtered and concentrated under reduced pressure to dryness. The residue was purified by prep-TLC (DCM:MeOH=10:1) and further purified by prep-HPLC to give Compound 221 (2.7 mg, yield 3.2%) as a white solid. ¹H NMR (400 MHz, CD₃OD) δ 8.51 (s, 1H), 8.18 (dd, J=12.9, 1.6 Hz, 1H), 7.38 (d, J=1.1 Hz, 1H), 7.25-7.21 (m, 2H), 6.91-6.86 (m, 3H), 5.26 (dd, J=10.6, 4.7 Hz, 1H), 4.57 (dd, J=9.4, 4.5 Hz, 1H), 4.02-3.88 (m, 8H), 3.73 (q, J=10.7 Hz, 2H), 2.43 (m, J=11.0, 7.5, 3.9 Hz, 3H), 2.17 (dd, J=13.1, 4.6 Hz, 1H), 2.06-1.96 (m, 3H), 1.91-1.79 (m, 1H), 1.69-1.61 (m, 1H), 0.97 (dd, J=12.5, 6.6 Hz, 6H). LC/MS (ESI) m/z: 586 (M+H)⁺.

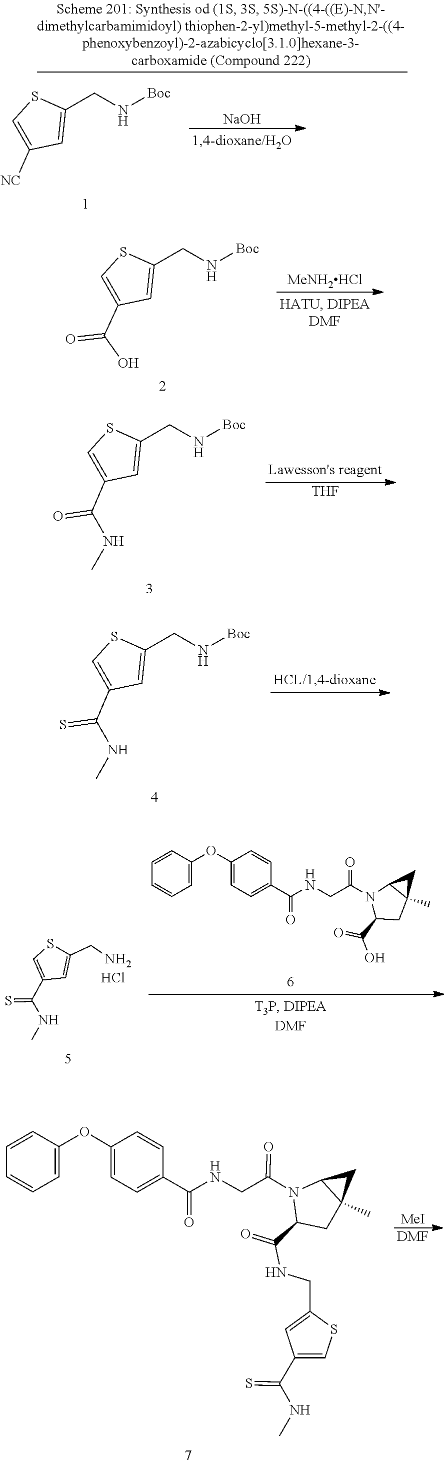

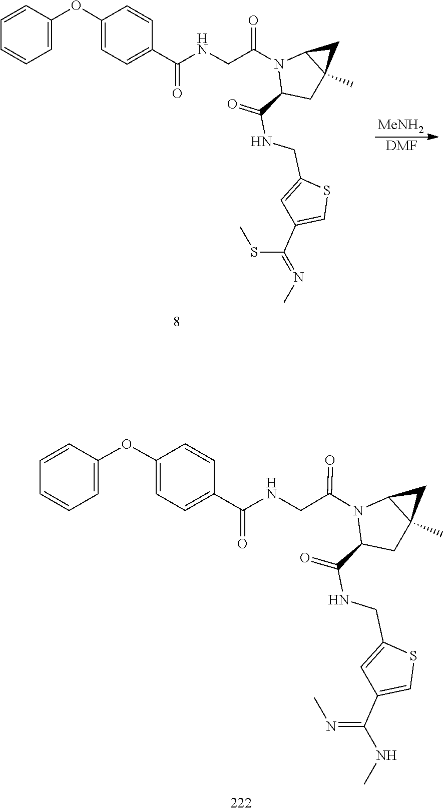

### Step 1: 5-(((tert-butoxycarbonyl)amino)methyl)thiophene-3-carboxylic acid (2)

To a solution of tert-butyl ((4-cyanothiophen-2-yl)methyl)carbamate (2.0 g, 8.39 mmol) in 1,4-dioxane (10 mL) was added a solution of NaOH (1.0 g, 25.17 mmol) in water (10 mL), and the mixture was stirred at 100° C. for 16 hours. The mixture was acidified with 0.5 M aq.HCl to pH-5 and extracted with EtOAc twice. The combined organic layers were washed with brine, dried over anhydrous Na₂SO₄, filtered and concentrated to dryness to give 5-(((tert-butoxycarbonyl)amino)methyl)thiophene-3-carboxylic acid (1.0 g, yield 46.3%) as white solid. LC/MS (ESI) (m/z): 258 (M+H)⁺.

### Step 2: tert-butyl ((4-(methylcarbamoyl)thiophen-2-yl)methyl)carbamate (3)

To a mixture of 5-(((tert-butoxycarbonyl)amino)methyl)thiophene-3-carboxylic acid (1.0 g, 3.89 mmol) and methanamine hydrochloride (0.39 g, 5.83 mmol) in DMF (10 mL) was added DIPEA (1.51 g, 11.67 mmol) and HATU (2.22 g, 5.84 mmol) at 0° C. and the mixture was stirred at room temperature for 1 hour. The mixture was diluted with water and extracted with EtOAc twice. The combined organic layers were washed with brine, dried over anhydrous Na₂SO₄, filtered and concentrated under reduced pressure to dryness. The residue was purified by flash chromatography on silica gel (PE:EtOAc=3:1) to give tert-butyl ((4-(methylcarbamoyl)thiophen-2-yl)methyl)carbamate (1.05 g, yield 100%) as white solid. LC/MS (ESI) m/z: 271 (M+H)⁺.

### Step 3: tert-butyl ((4-(methylcarbamothioyl)thiophen-2-yl)methyl)carbamate (4)

To a mixture of tert-butyl ((4-(methylcarbamoyl)thiophen-2-yl)methyl)carbamate (1.05 g, 3.88 mmol) in THE (20 mL) was added Lawesson's reagent (3.14 g, 7.76 mmol) at 0° C. and the mixture was stirred under N₂ atmosphere at room temperature for 24 hours. The mixture was filtered and the filtrate was concentrated under reduced pressure to dryness. The residue was purified by flash chromatography on silica gel (PE:EtOAc=3:1) to give tert-butyl ((4-(methylcarbamothioyl) thiophen-2-yl) methyl)carbamate (890 mg, yield 80.1%) as white solid. LC/MS (ESI) m/z: 287 (M+H)⁺.

### Step 4: 5-(aminomethyl)-N-methylthiophene-3-carbothioamide hydrochloride (5)

To a solution of tert-butyl ((4-(methylcarbamothioyl)thiophen-2-yl)methyl)carbamate (470 mg, 1.64 mmol) in DCM (5 mL) was added HCl/1,4-dioxane (1.0 mL, 4 mmol) at 0° C. under N₂ atmosphere. The reaction mixture was stirred at room temperature for 1 hour. The mixture was concentrated under reduced pressure to dryness to give 5-(aminomethyl)-N-methylthiophene-3-carbothioamide hydrochloride (370 mg, yield 100%) as yellow solid, which was used directly in the next reaction without further purification. LC/MS (ESI) m/z: 187 (M+H)⁺.

### Step 5: (1S,3S,5S)-5-methyl-N-((4-(methylcarbamothioyl)thiophen-2-yl)methyl)-2-((4-phenoxybenzoyl)glycyl)-2-azabicyclo[3.1.0]hexane-3-carboxamide (6)

To a mixture of (1 S,3S,5S)-5-methyl-2-((4-phenoxybenzoyl)glycyl)-2-azabicyclo[3.1.0]hexane-3-carboxylic acid (178 mg, 0.45 mmol) and 5-(aminomethyl)-N-methylthiophene-3-carbothioamide hydrochloride (200 mg, 0.90 mmol) in DMF (2 mL) was added DIPEA (175 mg, 1.35 mmol) and T₃P (426 mg, 0.67 mmol, 50% wt. in EtOAc) at 0° C. under N₂ atmosphere and the mixture was stirred at room temperature for 1 hour. The mixture was diluted with EtOAc and washed with saturated aq.NaHCO₃ solution. The aqueous layer was extracted with EtOAc twice. The combined organic layers were washed with brine, dried over anhydrous Na₂SO₄, filtered and concentrated under reduced pressure to dryness. The residue was purified by flash chromatography on silica gel (DCM:MeOH=20:1) to give (1 S,3S,5S)-5-methyl-N-((4-(methylcarbamothioyl)thiophen-2-yl)methyl)-2-((4-phenoxybenzoyl)glycyl)-2-azabicyclo[3.1.0]hexane-3-carboxamide (245 mg, yield 96.8%) as colorless oil. LC/MS (ESI) m/z: 563 (M+H)⁺.

### Step 6: methyl (Z)-N-methyl-5-(((1S,3S,5S)-5-methyl-2-((4-phenoxybenzoyl)glycyl)-2-azabicyclo[3.1.0]hexane-3-carboxamido)methyl)thiophene-3-carbimidothioate (7)

To a mixture of (1 S,3S,5S)-5-methyl-N-((4-(methylcarbamothioyl)thiophen-2-yl) methyl)-2-((4-phenoxybenzoyl)glycyl)-2-azabicyclo[3.1.0]hexane-3-carboxamide (45 mg, 0.08 mmol) in DMF (1 mL) was added Mel (23 mg, 0.16 mmol) at 0° C. under N₂ atmosphere and the mixture was stirred at room temperature overnight. The mixture was used directly in the next reaction without further purification. LC/MS (ESI) (m/z): 577 (M+H)⁺.

### Step 7: (1S,3S,5S)-N-((4-((E)-N,N′-dimethylcarbamimidoyl)thiophen-2-yl)methyl)-5-methyl-2-((4-phenoxybenzoyl)glycyl)-2-azabicyclo[3.1.0]hexane-3-carboxamide (Compound 222)

To a mixture of methyl (Z)-N-methyl-5-(((1S,3S,5S)-5-methyl-2-((4-phenoxybenzoyl) glycyl)-2-azabicyclo[3.1.0]hexane-3-carboxamido)methyl)thiophene-3-carbimidothioate (23 mg, 0.04 mmol) in DMF (0.5 mL) was added MeNH₂/THF solution (0.1 mmol, 0.1 mL, 1 M in THF) at 0° C. and the mixture was stirred at room temperature overnight. The mixture was concentrated to dryness and the residue was purified by prep-HPLC to give Compound 222 (3.2 mg, yield 14.3%) as a white solid. ¹H NMR (400 MHz, CD₃OD) δ 8.48 (s, 1H), 7.92 (t, J=7.5 Hz, 1H), 7.87-7.84 (m, 2H), 7.44-7.39 (m, 2H), 7.24-7.18 (m, 2H), 7.08-7.05 (m, 2H), 7.03-6.99 (m, 2H), 4.81 (d, J=3.3 Hz, 1H), 4.60-4.53 (m, 2H), 4.38-4.23 (m, 2H), 3.49-3.38 (m, 1H), 2.99 (d, J=2.6 Hz, 3H), 2.94 (d, J=2.2 Hz, 3H), 2.55-2.35 (m, 1H), 2.16 (dd, J=13.4, 3.4 Hz, 1H), 1.30 (s, 3H), 1.16-1.11 (m, 1H), 0.82 (dd, J=17.3, 11.3 Hz, 1H). LC/MS (ESI) m/z: 560 (M+H)⁺.

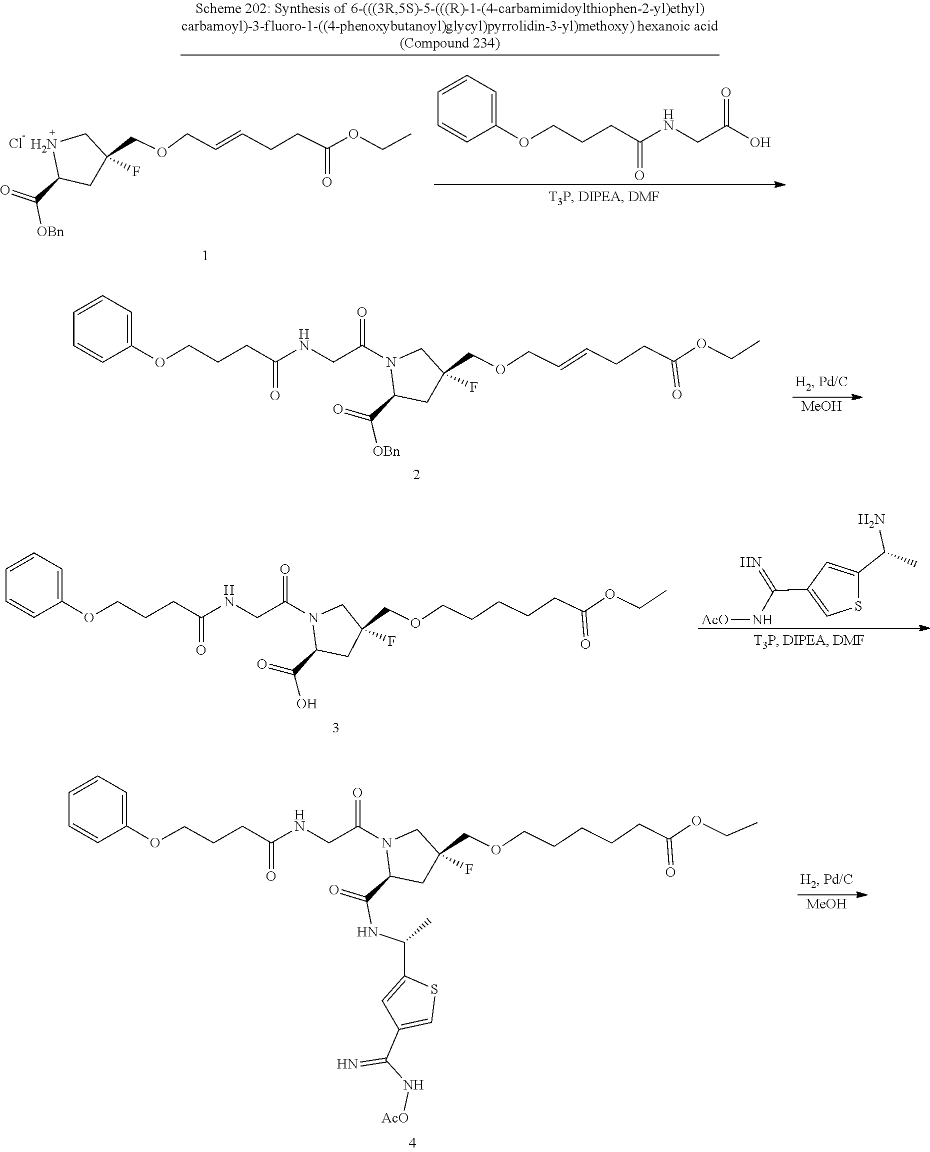

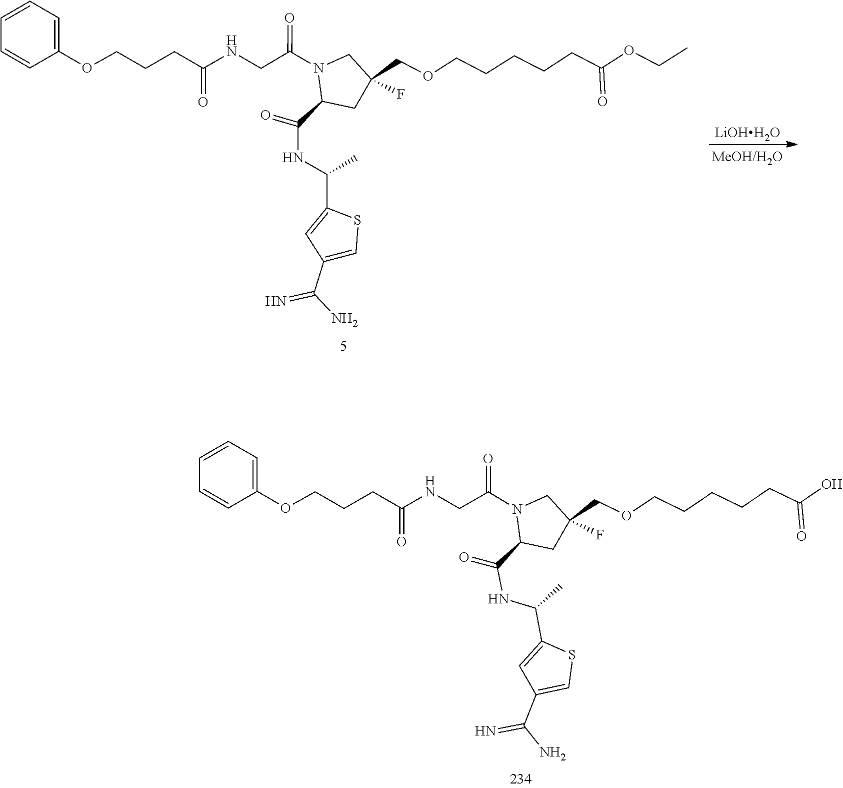 [SMILES: CCOC(=O)CCCCCOC[C@@]1(F)C[C@@H](C(=O)N[C@H](C)c2cc(C(=N)N)cs2)N(C(=O)CNC(=O)CCCOc2ccccc2)C1 ; "LiOH·H2O" ; C[C@@H](NC(=O)[C@@H]1C[C@](F)(COCCCCCC(=O)O)CN1C(=O)CNC(=O)CCCOc1ccccc1)c1cc(C(=N)N)cs1]

### Step 1: benzyl (2S,4R)-4-((((E)-6-ethoxy-6-oxohex-2-en-1-yl)oxy)methyl)-4-fluoro-1-((4-phenoxybutanoyl)glycyl)pyrrolidine-2-carboxylate

To a mixture of benzyl (2S,4R)-4-((((E)-6-ethoxy-6-oxohex-2-en-1-yl)oxy)methyl)-4-fluoropyrrolidine-2-carboxylate HCl salt (300 mg, 0.77 mmol) and (4-phenoxybutanoyl)glycine (120 mg, 0.51 mmol) in DMF (3.0 mL) was added DIPEA (0.42 mL, 2.55 mmol) and T₃P (966 mg, 1.53 mmol, 50% wt. in EtOAc) at 0° C. under N₂ atmosphere and the mixture was stirred at 25° C. for 2 hours. The mixture was diluted with EtOAc and washed with water twice. The combined organic layers were dried over anhydrous Na₂SO₄, filtered and concentrated to dryness under reduced pressure. The residue was purified by flash column chromatography on silica gel (DCM:MeOH=40:1) to give benzyl (2S,4R)-4-((((E)-6-ethoxy-6-oxohex-2-en-1-yl)oxy)methyl)-4-fluoro-1-((4-phenoxybutanoyl)glycyl)pyrrolidine-2-carboxylate (261 mg, yield 83.9%) as yellow oil. LC/MS (ESI) m/z: 613 (M+H)⁺.

### Step 2: (2S,4R)-4-(((6-ethoxy-6-oxohexyl)oxy)methyl)-4-fluoro-1-((4-phenoxybutanoyl)glycyl) pyrrolidine-2-carboxylic acid

To a solution of benzyl (2S,4R)-4-((((E)-6-ethoxy-6-oxohex-2-en-1-yl)oxy)methyl)-4-fluoro-1-((4-phenoxybutanoyl)glycyl)pyrrolidine-2-carboxylate (261 mg, 0.42 mmol) in MeOH (5 mL) was added Pd/C (10 mg, 10% wt.) at 0° C., and the reaction mixture was stirred at 25° C. under H₂ atmosphere for 2 hours. The mixture was filtered and concentrated to dryness under reduced pressure to give (2S,4R)-4-(((6-ethoxy-6-oxohexyl)oxy)methyl)-4-fluoro-1-((4-phenoxybutanoyl)glycyl)pyrrolidine-2-carboxylic acid (200 mg, yield 90.0%) as yellow oil, which was used directly in the next step. LC/MS (ESI) m/z: 525 (M+H)⁺.

### Step 3: ethyl 6-(((3R,5S)-5-(((R)-1-(4-(N-acetoxycarbamimidoyl)thiophen-2-yl)ethyl) carbamoyl)-3-fluoro-1-((4-phenoxybutanoyl)glycyl)pyrrolidin-3-yl)methoxy)hexanoate

To a mixture of (2S,4R)-4-(((6-ethoxy-6-oxohexyl)oxy)methyl)-4-fluoro-1-((4-phenoxybutanoyl) glycyl)pyrrolidine-2-carboxylic acid (170 mg, 0.32 mmol) and (R)-N-acetoxy-5-(1-aminoethyl)thiophene-3-carboximidamide (147 mg, 0.64 mmol) in DMF (5.0 mL) was added DIPEA (0.27 mL, 1.62 mmol) and T₃P (619 mg, 0.96 mmol, 50% wt. in EtOAc) at 0° C. under N₂ atmosphere and the mixture was stirred at 25° C. for 2 hours. The mixture was diluted with EtOAc and washed with water twice. The combined organic layers were dried over anhydrous Na₂SO₄, filtered and concentrated to dryness under reduced pressure. The residue was purified by prep-TLC (DCM:MeOH=20:1) to give ethyl 6-(((3R,5S)-5-(((R)-1-(4-(N-acetoxycarbamimidoyl)thiophen-2-yl)ethyl)carbamoyl)-3-fluoro-1-((4-phenoxybutanoyl) glycyl)pyrrolidin-3-yl)methoxy)hexanoate (117 mg, yield 49.4%) as yellow solid. LC/MS (ESI) m/z: 734 (M+H)⁺.

### Step 4: ethyl 6-(((3R,5S)-5-(((R)-1-(4-carbamimidoylthiophen-2-yl)ethyl)carbamoyl)-3-fluoro-1-((4-phenoxybutanoyl)glycyl)pyrrolidin-3-yl)methoxy)hexanoate

To a solution of ethyl 6-(((3R,5S)-5-(((R)-1-(4-(N-acetoxycarbamimidoyl)thiophen-2-yl)ethyl) carbamoyl)-3-fluoro-1-((4-phenoxybutanoyl)glycyl)pyrrolidin-3-yl)methoxy)hexanoate (117 mg, 0.16 mmol) in MeOH (5 mL) was added Pd/C (5 mg, 10% wt.) at 0° C., and the reaction mixture was stirred at 25° C. under H₂ atmosphere for 2 hours. The mixture was filtered and concentrated to dryness under reduced pressure to give ethyl 6-(((3R,5S)-5-(((R)-1-(4-carbamimidoylthiophen-2-yl)ethyl)carbamoyl)-3-fluoro-1-((4-phenoxybutanoyl)glycyl) pyrrolidin-3-yl)methoxy)hexanoate (100 mg, yield 93.4%) as a yellow solid, which was used directly in the next step. LC/MS (ESI) m/z: 676 (M+H)⁺.

### Step 5: 6-(((3R,5S)-5-(((R)-1-(4-carbamimidoylthiophen-2-yl)ethyl)carbamoyl)-3-fluoro-1-((4-phenoxybutanoyl)glycyl)pyrrolidin-3-yl)methoxy)hexanoic acid (Compound 234)

To a solution of ethyl 6-(((3R,5S)-5-(((R)-1-(4-carbamimidoylthiophen-2-yl)ethyl)carbamoyl)-3-fluoro-1-((4-phenoxybutanoyl)glycyl)pyrrolidin-3-yl)methoxy)hexanoate (100 mg, 0.15 mmol) in MeOH (3 mL) and water (1 mL) was added lithium hydroxide (9 mg, 0.23 mmol) at 0° C. and the mixture was stirred at room temperature for 2 hours. The mixture was acidified with 1N aq.HCl to pH-3. The residue was purified by prep-HPLC to give Compound 234 (18 mg, yield 24.2%) as a white solid. ¹H NMR (400 MHz, CD₃OD) δ 8.49 (s, 1H), 8.24 (t, J=2.5 Hz, 1H), 7.55 (d, J=10.9 Hz, 1H), 7.26-7.20 (m, 2H), 6.92-6.86 (m, 3H), 5.28 (dq, J=13.7, 6.9 Hz, 1H), 4.62 (dt, J=17.3, 8.2 Hz, 1H), 4.12-3.80 (m, 6H), 3.70 (dd, J=8.8, 4.2 Hz, 1H), 3.65 (dd, J=14.4, 7.4 Hz, 1H), 3.52 (t, J=6.3 Hz, 2H), 2.57-2.42 (m, 3H), 2.26 (t, J=7.4 Hz, 2H), 2.23-2.01 (m, 3H), 1.65-1.56 (m, 7H), 1.45-1.37 (m, 2H). LC/MS (ESI) m/z: 648 (M+H)⁺.

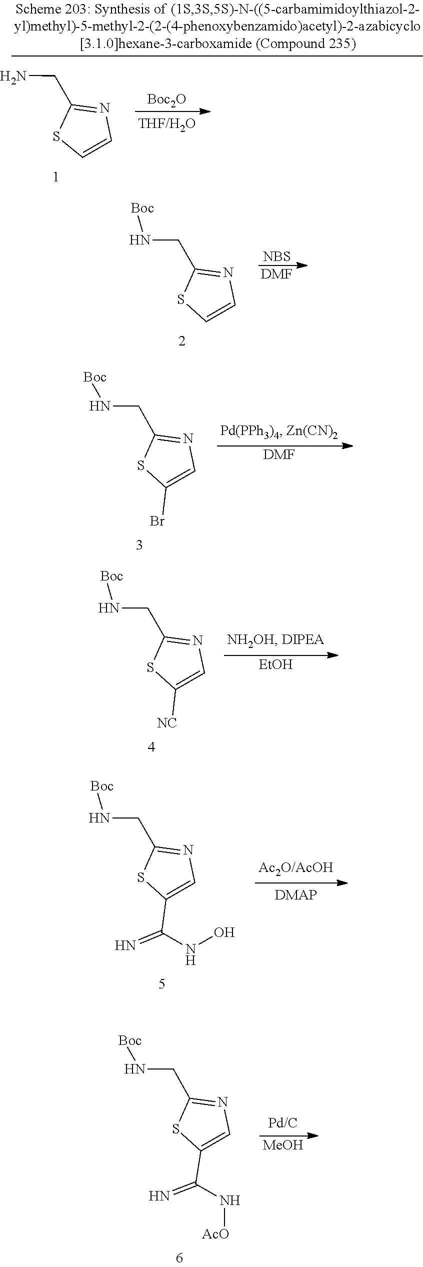

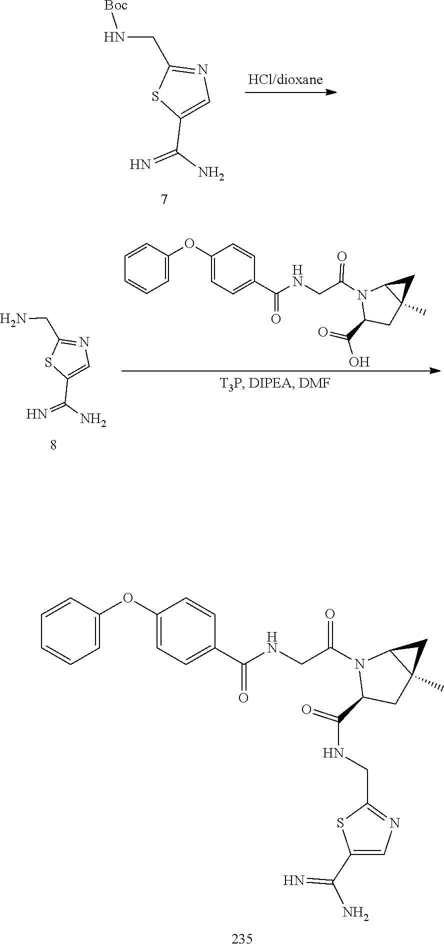 [SMILES: CC(C)(C)OC(=O)NCc1ncc(C(=N)N)s1 ; N=C(N)c1cnc(CN)s1 ; C[C@@]12C[C@@H]1N(C(=O)CNC(=O)c1ccc(Oc3ccccc3)cc1)[C@H](C(=O)O)C2 ; C[C@@]12C[C@@H]1N(C(=O)CNC(=O)c1ccc(Oc3ccccc3)cc1)[C@H](C(=O)NCc1ncc(C(=N)N)s1)C2]

### Step 1: tert-butyl (thiazol-2-ylmethyl)carbamate (2)

To a solution of thiazol-2-ylmethanamine (1 g, 8.77 mmol) in THE and water (5 mL) was added NaHCO₃ (2.2 g, 26.3 mmol) and BOC₂O (2.3 g, 10.5 mmol) and the mixture was stirred at 25° C. for 16 hours under N₂ atmosphere. The mixture was diluted with EtOAc, washed with water and brine, dried over anhydrous Na₂SO₄, filtered and concentrated under reduced pressure to dryness. The residue was purified by flash chromatography on silica gel (PE:EtOAc=10:1) to give tert-butyl (thiazol-2-ylmethyl)carbamate (1.6 g, yield 85.6%) as colorless oil. LC/MS (ESI) m/z: 215 (M+H)⁺.

### Step 2: tert-butyl ((5-bromothiazol-2-yl)methyl)carbamate (3)

To a solution of tert-butyl (thiazol-2-ylmethyl)carbamate (1.66 g, 7.75 mmol) in DMF (10 mL) was added NBS (1.5 g, 8.52 mmol) under N₂ atmosphere and the reaction mixture was stirred at room temperature for 3 hours. The mixture was diluted with EtOAc and washed with water and brine, dried over anhydrous Na₂SO₄, filtered and concentrated under reduced pressure to dryness. The residue was purified by flash chromatography on silica gel (PE:EtOAc=3:1) to give tert-butyl ((5-bromothiazol-2-yl)methyl)carbamate (1.86 g, yield 81.9%) as white solid. LC/MS (ESI) m/z:293 (M+H)⁺.

### Step 3: tert-butyl ((5-cyanothiazol-2-yl)methyl)carbamate (4)

To a solution of tert-butyl ((5-bromothiazol-2-yl)methyl)carbamate (690 mg, 2.35 mmol) in NMP (20 mL) was added Zn(CN)₂ (553 mg, 4.71 mmol) and Pd(PPh₃)₄ (408 mg, 0.35 mmol) under N₂ atmosphere and the reaction mixture was stirred at 120° C. overnight. The mixture was diluted with EtOAc and washed with water and brine, dried over anhydrous Na₂SO₄, filtered and concentrated under reduced pressure to dryness. The residue was purified by flash chromatography on silica gel (PE:EtOAc=5:1) to give tert-butyl ((5-cyanothiazol-2-yl)methyl)carbamate (140 mg, yield 24.9%) as white solid. LC/MS (ESI) m/z: 240 (M+H)⁺.

### Step 4: tert-butyl ((5-(N-hydroxycarbamimidoyl)thiazol-2-yl)methyl)carbamate (5)

To a solution of tert-butyl ((5-cyanothiazol-2-yl)methyl)carbamate (140 mg, 0.59 mmol) in EtOH (5 mL) was added NH₂OH·HCl (102 mg, 1.46 mmol) and DIPEA (227 mg, 1.76 mmol) under N₂ atmosphere and the reaction mixture was stirred at room temperature for 3 hours. The mixture was diluted with EtOAc, washed with water and brine, dried over anhydrous Na₂SO₄, filtered and concentrated under reduced pressure to dryness. The residue was purified by flash chromatography on silica gel (DCM:MeOH=10:1) to give tert-butyl ((5-(N-hydroxycarbamimidoyl)thiazol-2-yl)methyl)carbamate (155 mg, yield 97.3%) as white solid. LC/MS (ESI) m/z: 273 (M+H)⁺.

### Step 5: tert-butyl ((5-(N-acetoxycarbamimidoyl)thiazol-2-yl)methyl)carbamate (6)

To a solution of tert-butyl ((5-(N-hydroxycarbamimidoyl)thiazol-2-yl)methyl)carbamate (155 mg, 0.57 mmol) in AcOH (1 mL) was added DMAP (5 mg) and Ac₂O (1 mL) under N₂ atmosphere and the reaction mixture was stirred at room temperature overnight. The mixture was diluted with EtOAc, washed with water and brine, dried over anhydrous Na₂SO₄, filtered and concentrated under reduced pressure to dryness. The residue was purified by flash chromatography on silica gel (PE:EtOAc=1:1) to give tert-butyl ((5-(N-acetoxycarbamimidoyl)thiazol-2-yl)methyl)carbamate (140 mg, yield 78.2%) as white solid. LC/MS (ESI) m/z: 315 (M+H)⁺.

### Step 6: tert-butyl ((5-carbamimidoylthiazol-2-yl)methyl)carbamate (7)

To a solution of tert-butyl ((5-(N-acetoxycarbamimidoyl)thiazol-2-yl)methyl)carbamate (140 mg, 0.45 mmol) in MeOH (3 mL) was added Pd/C (100 mg, 10% wt.) and the reaction mixture was stirred under a H₂ balloon at room temperature overnight. The mixture was filtered and the filtrate was concentrated under reduced pressure to dryness. The residue was purified by prep-TLC (DCM:MeOH=10:1) to give tert-butyl ((5-carbamimidoylthiazol-2-yl)methyl)carbamate (110 mg, yield 96.4%) as white solid.

LC/MS (ESI) m/z: 257 (M+H)⁺.

### Step 7: 2-(aminomethyl)thiazole-5-carboximidamide (8)

To a solution of tert-butyl ((5-carbamimidoylthiazol-2-yl)methyl)carbamate (110 mg, 0.43 mmol) in 1,4-dioxane (2 mL) was added HCl/1,4-dioxane (1 mL) under N₂ atmosphere and the reaction mixture was stirred at room temperature for 3 hours. The mixture was concentrated to dryness under reduced pressure to give 2-(aminomethyl)thiazole-5-carboximidamide (66 mg, yield 98.5%) as a white solid. LC/MS (ESI) m/z:157 (M+H)⁺.

### Step 8: (1S,3S,5S)-N-((5-carbamimidoylthiazol-2-yl)methyl)-5-methyl-2-((4-phenoxybenzoyl) glycyl)-2-azabicyclo[3.1.0]hexane-3-carboxamide (Compound 235)

To a mixture of (1 S,3S,5S)-5-methyl-2-((4-phenoxybenzoyl)glycyl)-2-azabicyclo[3.1.0]hexane-3-carboxylic acid (83 mg, 0.21 mmol) and 2-(aminomethyl)thiazole-5-carboximidamide (33 mg, 0.21 mmol) in DMF (5 mL) was added DIPEA (164 mg, 1.27 mmol) and T₃P (202 mg, 0.32 mmol, 50% wt. in EtOAc) under N₂ atmosphere and the reaction mixture was stirred at room temperature for 2 hours. The mixture was diluted with EtOAc and washed with saturated aq.NaHCO₃ solution and brine, dried over anhydrous Na₂SO₄, filtered and concentrated under reduced pressure to dryness. The residue was purified by prep-TLC (DCM:MeOH=5:1) and further purified by prep-HPLC to give Compound 235 (10 mg, Yield 8.9%) as a white solid. ¹H NMR (400 MHz, CD₃OD) δ 8.54 (s, 1H), 8.43-8.38 (m, 1H), 7.86-7.81 (m, 2H), 7.42 (t, J=8.0 Hz, 2H), 7.21 (t, J=7.4 Hz, 1H), 7.08-6.98 (m, 4H), 4.91 (d, J=3.5 Hz, 1H), 4.73 (dd, J=11.6, 6.3 Hz, 2H), 4.42-4.30 (m, 2H), 3.49-3.42 (m, 1H), 2.47 (t, J=12.6 Hz, 1H), 2.25-2.09 (m, 1H), 1.33 (d, J=3.9 Hz, 3H), 1.23 (dd, J=5.8, 2.4 Hz, 1H), 0.85 (t, J=5.8 Hz, 1H). LC/MS (ESI) m/z:533 (M+H)⁺.

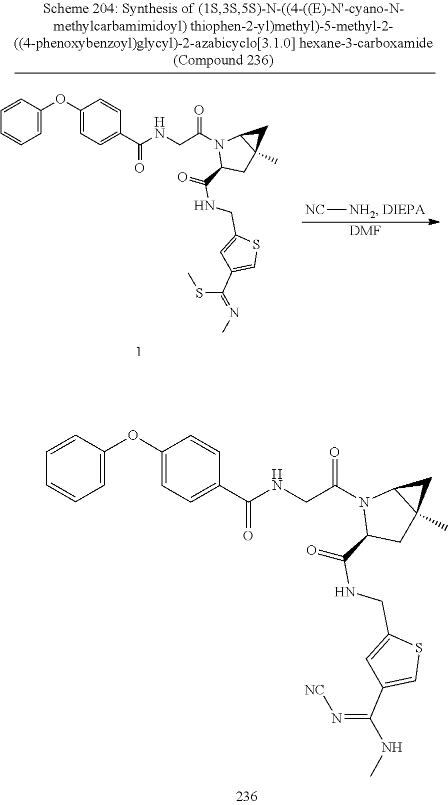

### Step 1: (1S,3S,5S)-N-((4-((E)-N′-cyano-N-methylcarbamimidoyl)thiophen-2-yl)methyl)-5-methyl-2-((4-phenoxybenzoyl)glycyl)-2-azabicyclo[3.1.0]hexane-3-carboxamide (Compound 236)

To a mixture of methyl (Z)-N-methyl-5-(((1S,3S,5S)-5-methyl-2-((4-phenoxybenzoyl)glycyl)-2-azabicyclo[3.1.0]hexane-3-carboxamido)methyl)thiophene-3-carbimidothioate (23 mg, 0.04 mmol) and DIPEA (16 mg, 0.12 mmol) in DMF (0.25 mL) was added cyanamide (4 mg, 0.1 mmol) at 0° C. and the mixture was stirred at room temperature overnight. The mixture was purified by prep-HPLC to give Compound 236 (3.3 mg, yield 14.5%) as a white solid. ¹HNMR (400 MHz, DMSO) δ 8.93 (d, J=4.5 Hz, 1H), 8.78-8.21 (m, 2H), 8.05 (dd, J=6.6, 1.5 Hz, 1H), 7.90 (d, J=8.8 Hz, 2H), 7.45 (t, J=7.9 Hz, 2H), 7.37-7.29 (m, 1H), 7.22 (t, J=7.4 Hz, 1H), 7.10 (d, J=7.7 Hz, 2H), 7.04 (d, J=8.8 Hz, 2H), 4.70-4.48 (m, 1H), 4.45-4.34 (m, 2H), 4.03 (dd, J=16.5, 5.4 Hz, 1H), 3.57-3.42 (m, 2H), 2.83 (dd, J=8.2, 3.8 Hz, 3H), 2.36-2.25 (m, 1H), 2.03-1.92 (m, 1H), 1.23 (d, J=5.7 Hz, 3H), 1.14-0.45 (m, 2H). LC/MS (ESI) m/z: 571 (M+H)⁺.

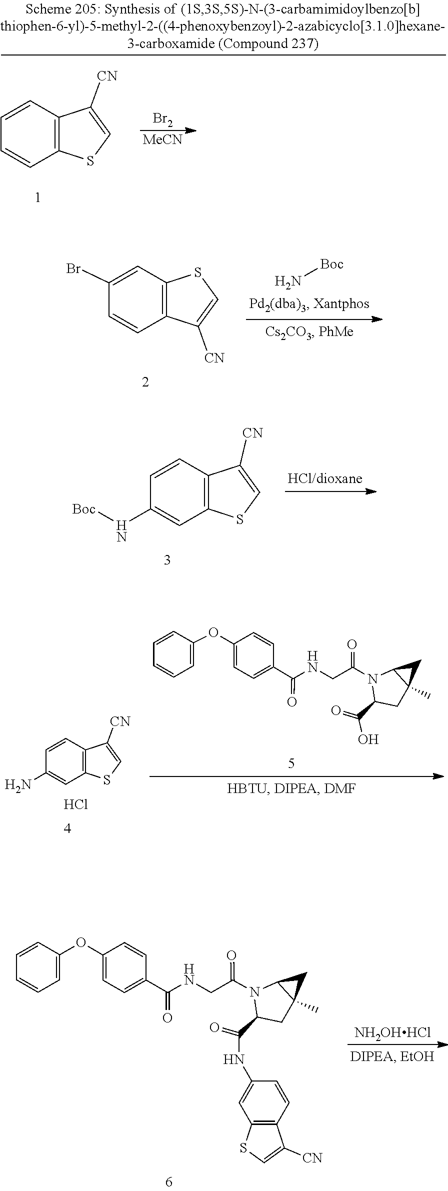

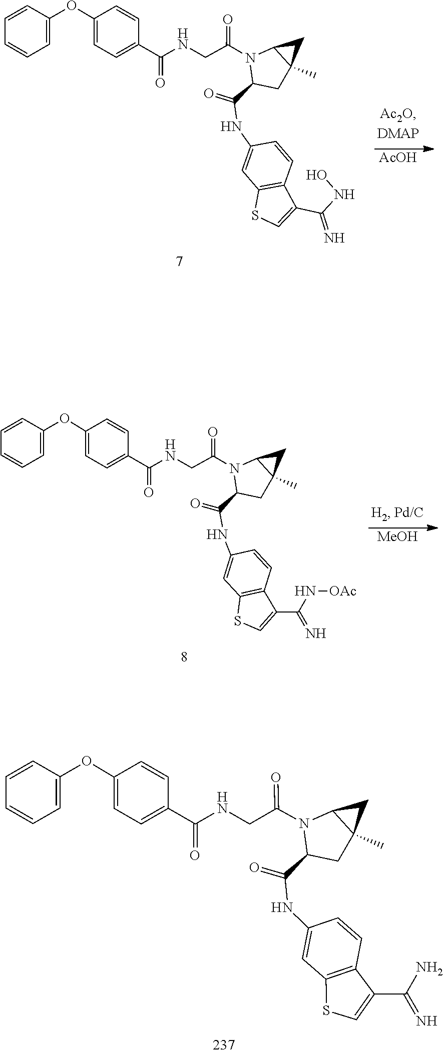 [SMILES: "AcOH" ; C[C@@]12C[C@@H]1N(C(=O)CNC(=O)c1ccc(Oc3ccccc3)cc1)[C@H](C(=O)Nc1ccc3c(C(=N)NO)csc3c1)C2 ; "MeOH" ; CC(=O)ONC(=N)c1csc2cc(NC(=O)[C@@H]3C[C@]4(C)C[C@@H]4N3C(=O)CNC(=O)c3ccc(Oc4ccccc4)cc3)ccc12 ; C[C@@]12C[C@@H]1N(C(=O)CNC(=O)c1ccc(Oc3ccccc3)cc1)[C@H](C(=O)Nc1ccc3c(C(=N)N)csc3c1)C2]

### Step 1: 6-bromobenzo[b]thiophene-3-carbonitrile (2)

To a mixture of benzo[b]thiophene-3-carbonitrile (1.0 g, 6.28 mmol) in MeCN (10 mL) was added Br₂ (804 mg, 5.02 mmol) in MeCN (4 mL) dropwise at 0° C. under N₂ atmosphere and the mixture was stirred at room temperature for 1 hour. The mixture was diluted with EtOAc and washed with saturated Na₂S₂O₃ solution. The aqueous layer was extracted with EtOAc twice. The combined organic layers were washed with brine, dried over anhydrous Na₂SO₄, filtered and concentrated under reduced pressure to dryness. The residue was purified by flash chromatography on silica gel (PE:EtOAc=3:1) to give crude of 6-bromobenzo[b]thiophene-3-carbonitrile (1.0 g, yield 66.9%) as yellow solid. LC/MS (ESI) m/z: 238 (M+H)⁺.

### Step 2: tert-butyl (3-cyanobenzo[b]thiophen-6-yl)carbamate (3)

To a mixture of 6-bromobenzo[b]thiophene-3-carbonitrile (1.0 g, yield 66.9%) in toluene (15 mL) was added tert-butyl carbamate (804 mg, 5.02 mmol), Xantphos (804 mg, 5.02 mmol), Cs₂CO₃ (804 mg, 5.02 mmol) and Pd₂(dba)₃ (4 mL) at 0° C. under N₂ atmosphere and the mixture was stirred at room temperature for 1 hour. The mixture was diluted with EtOAc and washed with saturated aq.Na₂S₂O₃ solution. The aqueous layer was extracted with EtOAc twice. The combined organic layers were washed with brine, dried over anhydrous Na₂SO₄, filtered and concentrated under reduced pressure to dryness. The residue was purified by flash chromatography on silica gel (PE:EtOAc=3:1) to give crude tert-butyl (3-cyanobenzo[b]thiophen-6-yl)carbamate (700 mg, yield 66.9%) as yellow solid. LC/MS (ESI) m/z: 275 (M+H)⁺.

### Step 3: 6-aminobenzo[b]thiophene-3-carbonitrile hydrochloride (4)

To a solution of tert-butyl (3-cyanobenzo[b]thiophen-6-yl)carbamate (700 mg, 2.55 mmol) in DCM (7 mL) was added HCl/1,4-dioxane (7.0 mL, 28 mmol) at 0° C. under N₂ atmosphere. The reaction mixture was stirred at room temperature for 1 hour. The mixture was concentrated under reduced pressure to dryness to give 6-aminobenzo[b]thiophene-3-carbonitrile hydrochloride (170 mg, yield 31.7%) as yellow solid, which was used directly in the next reaction without further purification. LC/MS (ESI) m/z: 175 (M+H)⁺.

### Step 4: (1S,3S,5S)-N-(3-cyanobenzo[b]thiophen-6-yl)-5-methyl-2-((4-phenoxybenzoyl) glycyl)-2-azabicyclo[3.1.0]hexane-3-carboxamide (6)

To a mixture of (1 S,3S,5S)-5-methyl-2-((4-phenoxybenzoyl)glycyl)-2-azabicyclo[3.1.0]hexane-3-carboxylic acid (120 mg, 0.32 mmol) and 6-aminobenzo[b]thiophene-3-carbonitrile hydrochloride (100 mg, 0.48 mmol) in DMF (2 mL) was added DIPEA (129 mg, 1.0 mmol) and T₃P (306 mg, 0.48 mmol, 50% wt. in EtOAc) at 0° C. under N₂ atmosphere and the mixture was stirred at room temperature for 1 hour. The mixture was diluted with EtOAc and washed with saturated aq.NaHCO₃ solution. The aqueous layer was extracted with EtOAc twice. The combined organic layers were washed with brine, dried over anhydrous Na₂SO₄, filtered and concentrated under reduced pressure to dryness. The residue was purified by flash chromatography on silica gel (DCM:MeOH=20:1) to give (1 S,3S,5S)-N-(3-cyanobenzo[b]thiophen-6-yl)-5-methyl-2-((4-phenoxybenzoyl)glycyl)-2-azabicyclo[3.1.0]hexane-3-carboxamide (115 mg, yield 65.3%) as oil. LC/MS (ESI) m/z: 551 (M+H)⁺.

### Step 5: (1S,3S,5S)-N-(3-(N-hydroxycarbamimidoyl)benzo[b]thiophen-6-yl)-5-methyl-2-((4-phenoxybenzoyl)glycyl)-2-azabicyclo[3.1.0]hexane-3-carboxamide (7)

To a mixture of (1 S,3S,5S)-N-(3-cyanobenzo[b]thiophen-6-yl)-5-methyl-2-((4-phenoxybenzoyl)glycyl)-2-azabicyclo[3.1.0]hexane-3-carboxamide (110 mg, 0.2 mmol) and DIPEA (78 mg, 0.6 mmol) in EtOH (5 mL) was added hydroxylamine hydrochloride (28 mg, 0.4 mmol) and the mixture was stirred at 90° C. overnight. The mixture was diluted with EtOAc and washed with saturated aq.NaHCO₃ solution. The aqueous layer was extracted with EtOAc twice. The combined organic layers were washed with brine, dried over anhydrous Na₂SO₄, filtered and concentrated under reduced pressure to dryness. The residue was purified by flash chromatography on silica gel (DCM:MeOH=20:1) to give (1S,3S,5S)-N-(3-(N-hydroxycarbamimidoyl)benzo[b]thiophen-6-yl)-5-methyl-2-((4-phenoxybenzoyl)glycyl)-2-azabicyclo[3.1.0]hexane-3-carboxamide (100 mg, yield 85.7%) as white solid. LC/MS (ESI) m/z: 584 (M+H)⁺.

### Step 6: (1S,3S,5S)-N-(3-(N-acetoxycarbamimidoyl)benzo[b]thiophen-6-yl)-5-methyl-2-((4-phenoxybenzoyl)glycyl)-2-azabicyclo[3.1.0]hexane-3-carboxamide (8)

To a mixture of (1 S,3S,5S)-N-(3-(N-hydroxycarbamimidoyl)benzo[b]thiophen-6-yl)-5-methyl-2-((4-phenoxybenzoyl)glycyl)-2-azabicyclo[3.1.0]hexane-3-carboxamide (100 mg, 0.17 mmol) and Ac₂O (1 mL) in AcOH (1 mL) was added DMAP (12 mg, 0.1 mmol) and the mixture was stirred at room temperature for 2 hours. The mixture was diluted with EtOAc and washed with saturated aq.NaHCO₃ solution. The aqueous layer was extracted with EtOAc twice. The combined organic layers were washed with brine, dried over anhydrous Na₂SO₄, filtered and concentrated under reduced pressure to dryness. The residue was purified by flash chromatography on silica gel (DCM:MeOH=20:1) to give (1S,3S,5S)-N-(3-(N-acetoxycarbamimidoyl)benzo[b]thiophen-6-yl)-5-methyl-2-((4-phenoxybenzoyl)glycyl)-2-azabicyclo[3.1.0]hexane-3-carboxamide (60 mg, yield 56.5%) as a yellow solid. LC/MS (ESI) m/z: 626 (M+H)⁺.

### Step 7: (1S,3S,5S)-N-(3-carbamimidoylbenzo[b]thiophen-6-yl)-5-methyl-2-((4-phenoxybenzoyl)glycyl)-2-azabicyclo[3.1.0]hexane-3-carboxamide (Compound 237)

To a mixture of (1 S,3S,5S)-N-(3-(N-acetoxycarbamimidoyl)benzo[b]thiophen-6-yl)-5-methyl-2-((4-phenoxybenzoyl)glycyl)-2-azabicyclo[3.1.0]hexane-3-carboxamide (50 mg, 0.08 mmol) in MeOH (5 mL) was added Pd/C (10 mg, 10% wt.) at 0° C. under H₂ atmosphere and the mixture was stirred at room temperature overnight. The mixture was filtered and concentrated under reduced pressure to dryness. The residue was purified by prep-HPLC to give Compound 237 (8.3 mg, yield 18.2%) as a white solid. ¹H NMR (400 MHz, CD₃OD) δ 8.44 (s, 1H), 8.31 (s, 1H), 7.93 (d, J=8.8 Hz, 1H), 7.90 (s, 1H), 7.88 (s, 1H), 7.66 (d, J=6.9 Hz, 1H), 7.41 (s, 2H), 7.21 (d, J=7.4 Hz, 1H), 7.06 (d, J=7.7 Hz, 2H), 7.01 (d, J=8.8 Hz, 2H), 4.99 (s, 1H), 4.49 (d, J=16.6 Hz, 1H), 4.31 (d, J=16.7 Hz, 1H), 3.47 (d, J=11.7 Hz, 2H), 2.35 (s, 1H), 1.35 (s, 3H), 1.29 (s, 1H), 0.86 (s, 1H). LC/MS (ESI) m/z: 571 (M+H)⁺.

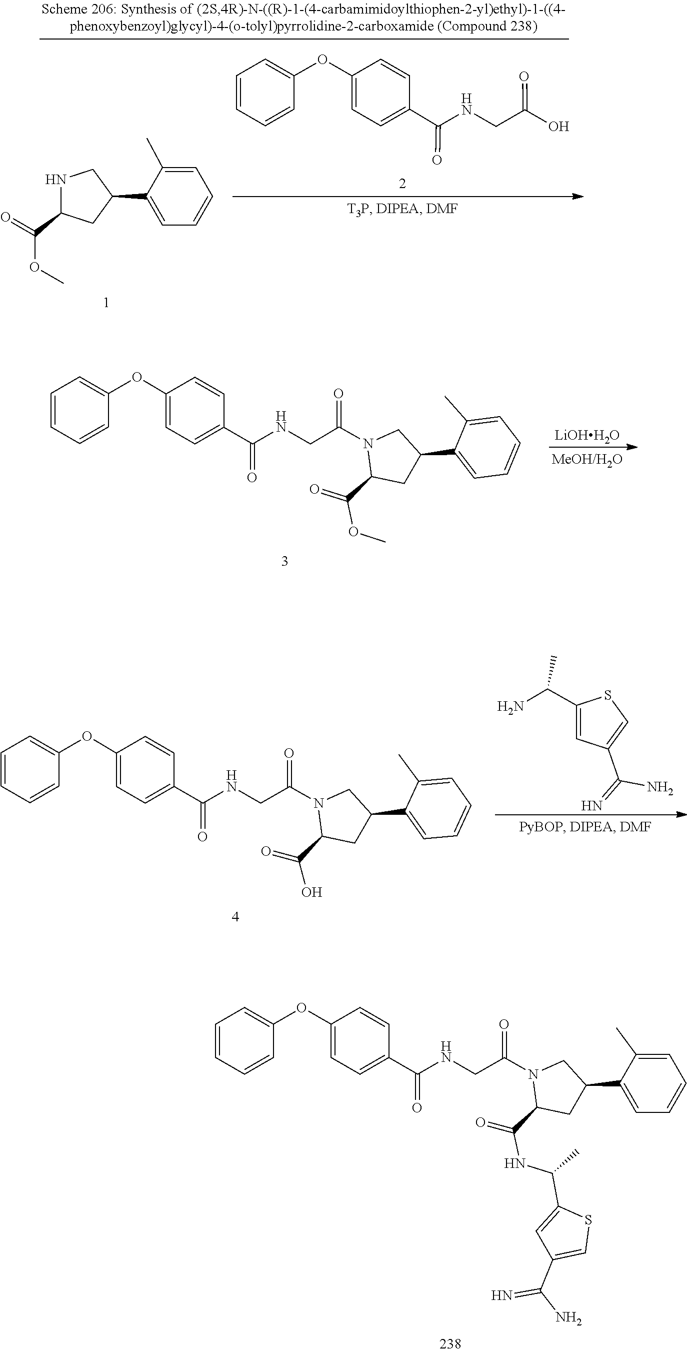

### Step 1: methyl (S)-1-((4-phenoxybenzoyl)glycyl)-4-(o-tolyl)pyrrolidine-2-carboxylate

To a mixture of methyl (S)-4-(o-tolyl)pyrrolidine-2-carboxylate (60 mg, 0.27 mmol) and (4-phenoxybenzoyl)glycine (73 mg, 0.27 mmol) in DMF (3 mL) was added DIPEA (209 mg, 1.6 mmol) and T₃P (515 mg, 0.81 mmol, 50% in EtOAc) at 0° C. The mixture was stirred under nitrogen atmosphere at room temperature for 1 hour. The mixture was quenched with saturated aq.NaHCO₃ solution and extracted with EtOAc twice. The combined organic layers were washed with brine, dried over anhydrous Na₂SO₄, filtered and concentrated to dryness under reduced pressure. The residue was purified by flash chromatography on silica gel (PE:EtOAc=1:1) to give methyl (S)-1-((4-phenoxybenzoyl)glycyl)-4-(o-tolyl)pyrrolidine-2-carboxylate (50 mg, yield 38.7%) as a white solid. LCMS (ESI) m/z=473 (M+H)⁺.

### Step 2: (S)-1-((4-phenoxybenzoyl)glycyl)-4-(o-tolyl)pyrrolidine-2-carboxylic acid

To a solution of methyl (S)-1-((4-phenoxybenzoyl)glycyl)-4-(o-tolyl)pyrrolidine-2-carboxylate (50 mg, 0.10 mmol) in MeOH (3 mL)/water (1 mL) was added LiOH·H₂O (13 mg, 0.30 mmol) at 0° C. and the mixture was stirred at room temperature for 2 hours. The mixture was acidified with 1N aq.HCl to pH-3, extracted with EtOAc twice. The combined organic layers were washed with brine, dried over anhydrous Na₂SO₄, filtered and concentrated under reduced pressure to dryness to give (S)-1-((4-phenoxybenzoyl)glycyl)-4-(o-tolyl)pyrrolidine-2-carboxylic acid (30 mg, yield 62.5%) as a colorless oil, which was used directly in the next step. LC/MS (ESI) (m/z): 458 (M+H)⁺.

### Step 3: (2S,4R)-N-((R)-1-(4-carbamimidoylthiophen-2-yl)ethyl)-1-((4-phenoxybenzoyl) glycyl)-4-(o-tolyl)pyrrolidine-2-carboxamide (Compound 238)

To a mixture of (S)-1-((4-phenoxybenzoyl)glycyl)-4-(o-tolyl)pyrrolidine-2-carboxylic acid (11 mg, 0.060 mmol) in DMF (3 mL) was added DIPEA (23 mg, 0.18 mmol) and PyBOP (50 mg, 0.090 mmol) at 0° C. The mixture was stirred under nitrogen atmosphere at room temperature for 1 hour. The mixture was diluted with saturated aq.NaHCO₃ solution and extracted with EtOAc twice. The combined organic layers were washed with brine, dried over anhydrous Na₂SO₄, filtered and concentrated to dryness. The residue was purified by flash chromatography on silica gel (DCM:MeOH=1:1), prep-HPLC and SFC to give Compound 238 (5.0 mg, yield 13.9%) as a white solid. ¹H NMR (400 MHz, CD₃OD) δ 8.55 (s, 1H), 8.24 (d, J=1.5 Hz, 1H), 7.86 (d, J=8.8 Hz, 2H), 7.57 (s, 1H), 7.42 (t, J=8.0 Hz, 2H), 7.34 (d, J=7.6 Hz, 1H), 7.23-7.14 (m, 4H), 7.07 (d, J=7.6 Hz, 2H), 7.01 (d, J=8.8 Hz, 2H), 5.38-5.23 (m, 1H), 4.57-4.50 (m, 1H), 4.36 (d, J=16.7 Hz, 1H), 4.24-4.15 (m, 1H), 4.11 (d, J=16.6 Hz, 1H), 3.83-3.74 (m, 1H), 3.69-3.62 (m, 1H), 2.73-2.56 (m, 1H), 2.41 (d, J=15.0 Hz, 3H), 2.13-2.03 (m, 1H), 1.61 (t, J=11.4 Hz, 3H).LCMS (ESI) m/z=610 (M+H)⁺.

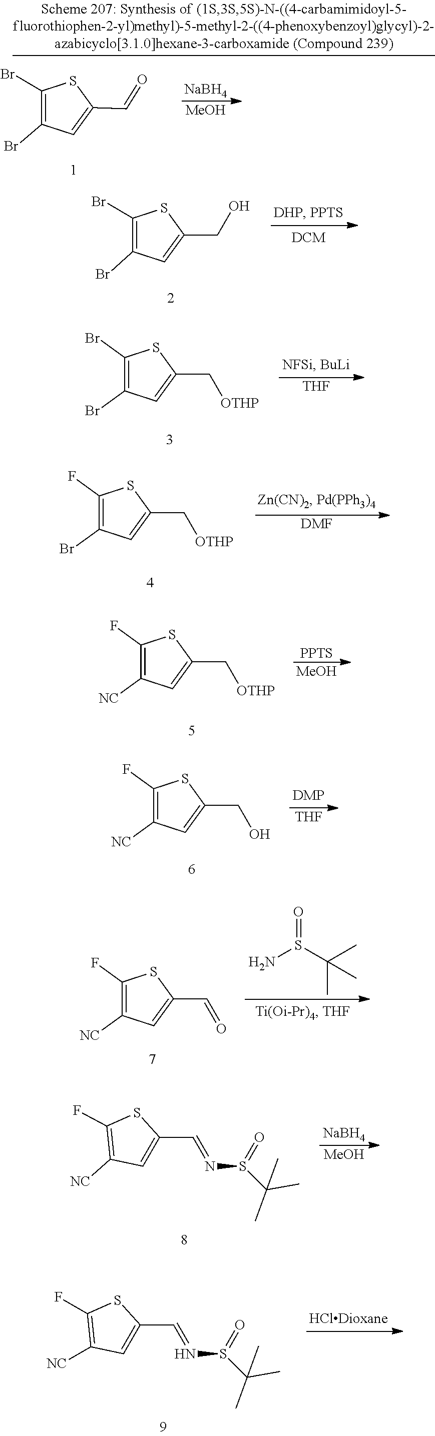

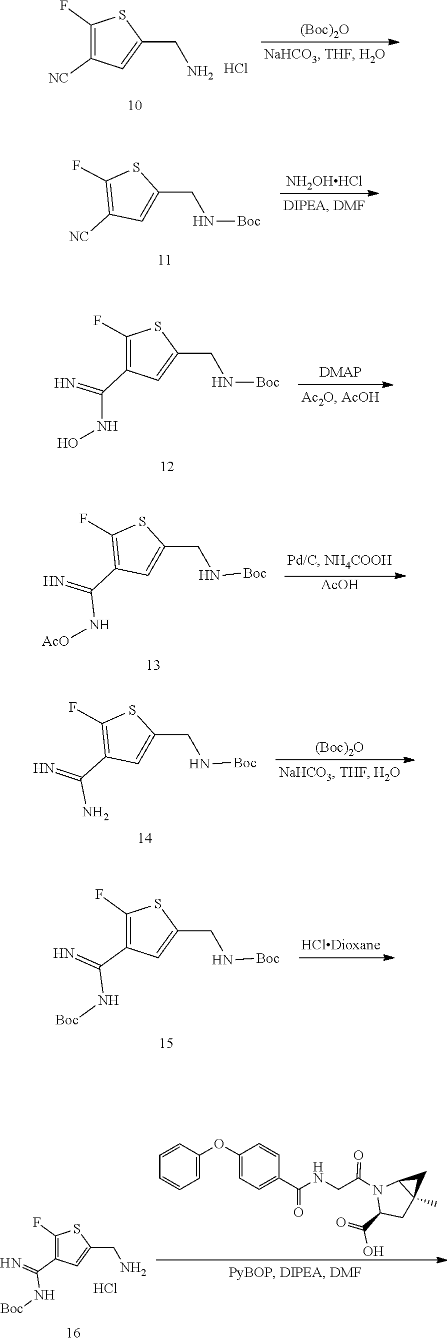

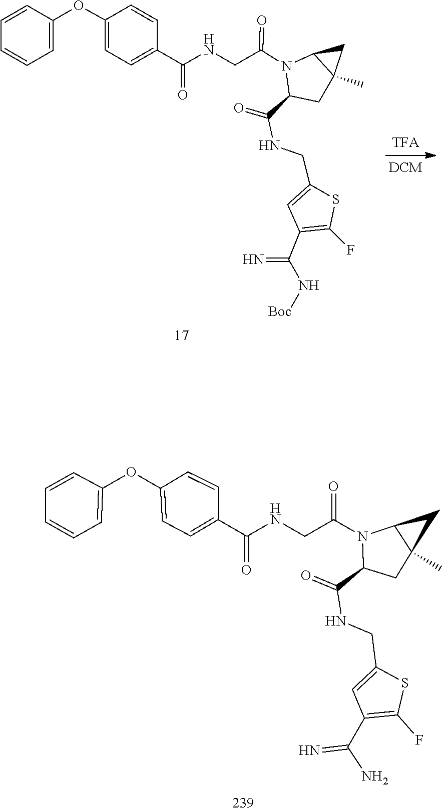 [SMILES: CC(C)(C)OC(=O)NC(=N)c1cc(CNC(=O)[C@@H]2C[C@]3(C)C[C@@H]3N2C(=O)CNC(=O)c2ccc(Oc3ccccc3)cc2)sc1F ; C[C@@]12C[C@@H]1N(C(=O)CNC(=O)c1ccc(Oc3ccccc3)cc1)[C@H](C(=O)NCc1cc(C(=N)N)c(F)s1)C2]

### Step 1: (4,5-dibromothiophen-2-yl)methanol (2)

To a mixture of 4,5-dibromothiophene-2-carbaldehyde (10.0 g, 37.45 mmol) in MeOH (90 mL) was added NaBH₄ (1.49 g, 39.33 mmol) at 0° C. and the reaction mixture was stirred at room temperature for 2 hours. After completion of the reaction, the reaction mixture was quenched with ice water and extracted with DCM twice. The combined organic layers were washed with brine, dried over anhydrous Na₂SO₄ and concentrated to dryness under reduced pressure. The residue was purified by flash chromatography on silica gel (PE:EtOAc=80:1) to give (4,5-dibromothiophen-2-yl)methanol (9.9 g, yield 98.3%) as white solid.

### Step 2: 2-((4,5-dibromothiophen-2-yl)methoxy)tetrahydro-2H-pyran (3)

To a solution of (4,5-dibromothiophen-2-yl)methanol (9.9 g, 36.67 mmol) in DCM (100 mL) was added PPTS (2.30 g, 9.17 mmol) and DHP (4.62 g, 55.01 mmol) at 0° C. and the reaction mixture was stirred at room temperature for 16 hours. The mixture was quenched with ice-water and extracted with DCM twice. The combined organic layers were washed with brine, dried over anhydrous Na₂SO₄, filtered and concentrated to dryness under reduced pressure. The residue was purified by flash chromatography on silica gel (PE:EtOAc=100:1) to give 2-((4,5-dibromothiophen-2-yl)methoxy)tetrahydro-2H-pyran (12.1 g, yield 93.2%) as a yellow oil.

### Step 3: 2-((4-bromo-5-fluorothiophen-2-yl)methoxy)tetrahydro-2H-pyran (4)

At −78° C., to a solution of t2-((4,5-dibromothiophen-2-yl)methoxy)tetrahydro-2H-pyran (6.38 g, 18.0 mmol) in THE (180 mL) was added n-BuLi (9.37 mL, 23.44 mmol, 2.5 M) at −78° C. and the resulting mixture was stirred under N₂ atmosphere for 0.5 hours. Then a solution of NFSI (7.37 g, 23.44 mmol) in THE (60 mL) was added and the mixture was stirred at −78° C. and warmed to −10° C. within 6 hours. The mixture was quenched with saturated aq.NH₄Cl solution and extracted with EtOAc twice. The combined organic layer were washed with brine, dried over anhydrous Na₂SO₄, filtered and concentrated to dryness under reduced pressure. The residue was purified by flash column chromatography on silica gel (PE:EtOAc=100:1) to give 2-((4-bromo-5-fluorothiophen-2-yl)methoxy)tetrahydro-2H-pyran (4.0 g, yield 75.5%) as a yellow oil.

### Step 4: 2-fluoro-5-(((tetrahydro-2H-pyran-2-yl)oxy)methyl)thiophene-3-carbonitrile (5)

To a solution of 2-((4-bromo-5-fluorothiophen-2-yl)methoxy)tetrahydro-2H-pyran (3.0 g, 10.2 mmol) in DMA (40 mL) was added Zn(CN)₂ (2.03 g, 17.35 mmol), dppf (2.83 g, 5.1 mmol), Zn (1.99 g, 30.6 mmol) and Pd₂(dba)₃ (1.76 g, 3.06 mmol). The mixture was degassed under N₂ atmosphere for three times and stirred at 90° C. for 12 hours. The mixture was diluted with EtOAc, washed with water and brine, dried over anhydrous Na₂SO₄, filtered and concentrated under reduced pressure to dryness. The residue was purified by flash chromatography on silica gel (PE:EtOAc=50:1) to give 2-fluoro-5-(((tetrahydro-2H-pyran-2-yl)oxy)methyl)thiophene-3-carbonitrile (2.0 g, yield 81.3%) as a yellow oil. ¹H NMR (400 MHz, CDCl₃) δ 6.77 (d, J=3.2 Hz, 1H), 4.72-4.69 (m, 2H), 4.56 (dd, J=12.8, 12.8 Hz, 1H), 3.87-3.82 (m, 1H), 3.59-3.53 (m, 1H), 1.86-1.69 (m, 2H), 1.68-1.58 (m, 3H), 1.56-1.50 (m, 1H).

### Step 5: 2-fluoro-5-(hydroxymethyl)thiophene-3-carbonitrile (6)

To a mixture of 2-fluoro-5-(((tetrahydro-2H-pyran-2-yl)oxy)methyl)thiophene-3-carbonitrile (2.0 g, 8.30 mmol) in MeOH (20 mL) was added PPTS (1.04 g, 4.15 mmol) at 0° C. and the reaction mixture was stirred at room temperature for 16 hours. After completion of the reaction, the reaction mixture was quenched with ice water and extracted with DCM twice. The combined organic layers were washed with brine, dried over anhydrous Na₂SO₄ and concentrated to dryness under reduced pressure. The residue was purified by flash chromatography on silica gel (PE:EtOAc=10:1) to give 2-fluoro-5-(hydroxymethyl)thiophene-3-carbonitrile (850 mg, yield 65.4%) as a brown oil.

### Step 6: 2-fluoro-5-formylthiophene-3-carbonitrile (7)

To a mixture of 2-fluoro-5-(hydroxymethyl)thiophene-3-carbonitrile (850 mg, 5.41 mmol) in THE (10 mL) was added DMP (2.75 g, 6.50 mmol) at 0° C. and the reaction mixture was stirred at room temperature for 4 hours. After completion of the reaction, the reaction mixture was quenched with ice water and extracted with DCM twice. The combined organic layers were washed with brine, dried over anhydrous Na₂SO₄ and concentrated to dryness under reduced pressure. The residue was purified by flash chromatography on silica gel (PE:EtOAc=20:1) to give 2-fluoro-5-formylthiophene-3-carbonitrile (670 mg, yield 79.9%) as a yellow oil. ¹H NMR (400 MHz, CDCl₃) δ 9.80 (d, J=4.0 Hz, 1H), 7.65 (d, J=3.2 Hz, 1H).

### Step 7: (S,E)-N-((4-cyano-5-fluorothiophen-2-yl)methylene)-2-methylpropane-2-sulfinamide (8)

To a solution of 2-fluoro-5-formylthiophene-3-carbonitrile (670 mg, 4.32 mmol) in THE (10 mL) was added (S)-2-methylpropane-2-sulfinamide (575 mg, 4.75 mmol) and titanium tetraisopropanolate (2.45 g, 8.64 mmol) at 0° C. and the reaction mixture was stirred at room temperature for 16 hours. The mixture was quenched with ice-water and extracted with DCM twice. The combined organic layers were washed with brine, dried over anhydrous Na₂SO₄, filtered and concentrated to dryness under reduced pressure. The residue was purified by flash chromatography on silica gel (PE:EtOAc=15:1) to give (S,E)-N-((4-cyano-5-fluorothiophen-2-yl)methylene)-2-methylpropane-2-sulfinamide (730 mg, yield 65.2%) as a yellow solid. LC/MS (ESI) m/z: 259(M+H)⁺.

### Step 8: (S)-N-((4-cyano-5-fluorothiophen-2-yl)methyl)-2-methylpropane-2-sulfinamide (9)

To a mixture of (S,E)-N-((4-cyano-5-fluorothiophen-2-yl)methylene)-2-methylpropane-2-sulfinamide (730 mg, 2.83 mmol) in MeOH (10 mL) was added NaBH₄ (128 mg, 3.40 mmol) at 0° C. and the reaction mixture was stirred at 0° C. for 2 hours. After completion of the reaction, the reaction mixture was quenched with ice water and extracted with DCM twice. The combined organic layers were washed with brine, dried over anhydrous Na₂SO₄ and concentrated to dryness under reduced pressure. The residue was purified by flash chromatography on silica gel (PE:EtOAc=7:1) to give (S)-N-((4-cyano-5-fluorothiophen-2-yl)methyl)-2-methylpropane-2-sulfinamide (610 mg, yield 83.0%) as a light oil. LC/MS (ESI) m/z: 261 (M+H)⁺.

### Step 9: 5-(aminomethyl)-2-fluorothiophene-3-carbonitrile hydrochloride (10)

A solution of (S)-N-((4-cyano-5-fluorothiophen-2-yl)methyl)-2-methylpropane-2-sulfinamide (610 mg, 2.34 mmol) in HCl/1,4-dioxane (6 mL) was stirred at room temperature for 2 hours. The mixture was concentrated to dryness under reduced pressure to give methyl 5-(aminomethyl)-2-fluorothiophene-3-carbonitrile hydrochloride (440 mg, yield 97.8%) as a white solid, which was directly used in the next reaction without further purification. LC/MS (ESI) m/z: 157 (M+H)⁺.

### Step 10: tert-butyl ((4-cyano-5-fluorothiophen-2-yl)methyl)carbamate (11)

To a mixture of ethyl 5-(aminomethyl)-2-fluorothiophene-3-carbonitrile hydrochloride (230 mg, 1.20 mmol) and di-tert-butyl dicarbonate (522 mg, 2.40 mmol) in THE (3 mL) was added a solution of NaHCO₃ (1.01 g, 11.98 mmol) in H₂O (1 mL) drop-wise at 0° C. and the mixture was stirred at room temperature for 2 hours. The mixture was diluted with water and extracted with EtOAc twice. The combined organic layers were dried over anhydrous Na₂SO₄, filtered and concentrated under reduced pressure. The residue was purified by flash chromatography on silica gel (PE:EtOAc=12:1) to give tert-butyl ((4-cyano-5-fluorothiophen-2-yl)methyl)carbamate (300 mg, yield 98.0%) as a yellow solid.

LC/MS (ESI) m/z: 257 (M+H)⁺.

### Step 11: tert-butyl ((5-fluoro-4-(N-hydroxycarbamimidoyl)thiophen-2-yl)methyl)carbamate (12)

To a solution of tert-butyl ((4-cyano-5-fluorothiophen-2-yl)methyl)carbamate (300 mg, 1.17 mmol) in EtOH (5 mL) was added NH₂OH·HCl (203 mg, 2.93 mmol) and DIPEA (453 mg, 3.51 mmol) and the reaction mixture was stirred at room temperature overnight. The mixture was diluted with EtOAc, washed with water and brine, dried over anhydrous Na₂SO₄, filtered and concentrated under reduced pressure to dryness. The residue was purified by flash chromatography on silica gel (DCM:MeOH=60:1) to give tert-butyl ((5-fluoro-4-(N-hydroxycarbamimidoyl)thiophen-2-yl)methyl)carbamate (235 mg, yield 69.5%) as a white solid. LC/MS (ESI) m/z: 290 (M+H)⁺.

### Step 12: tert-butyl ((4-(N-acetoxycarbamimidoyl)-5-fluorothiophen-2-yl)methyl) carbamate(13)

To a mixture of tert-butyl ((5-fluoro-4-(N-hydroxycarbamimidoyl)thiophen-2-yl)methyl)carbamate (220 mg, 0.80 mmol) in AcOH (1.5 mL) was added Ac₂O (1.5 mL) and DMAP (29 mg) and the mixture was stirred at 30° C. for 5 hours. The mixture was diluted with DCM, washed with water and brine, dried over anhydrous Na₂SO₄, filtered and concentrated to dryness under reduced pressure. The residue was purified by flash chromatography on silica gel (DCM:MeOH=80:1) to give tert-butyl ((4-(N-acetoxycarbamimidoyl)-5-fluorothiophen-2-yl)methyl)carbamate (220 mg, yield 83.5%) as a colorless oil. LC/MS (ESI) m/z: 332 (M+H)⁺.

### Step 13: tert-butyl ((4-carbamimidoyl-5-fluorothiophen-2-yl)methyl)carbamate(14)

To a solution of tert-butyl ((4-(N-acetoxycarbamimidoyl)-5-fluorothiophen-2-yl)methyl)carbamate (170 mg, 0.51 mmol) in AcOH (20 mL) was added ammonium formate (161 mg, 2.57 mmol) and Pd/C (150 mg, 10% wt.) and the reaction mixture was stirred at 80° C. for 10 minutes under a N₂ atmosphere. The mixture was filtered and the filtrate was concentrated under reduced pressure to dryness. The residue was purified by prep-TLC (DCM:MeOH=20:1) to give tert-butyl ((4-carbamimidoyl-5-fluorothiophen-2-yl)methyl)carbamate (120 mg, yield 85.6%) as a light oil. LC/MS (ESI) m/z:274 (M+H)⁺.

### Step 14: tert-butyl ((4-(N-(tert-butoxycarbonyl)carbamimidoyl)-5-fluorothiophen-2-yl)methyl)carbamate (15)

To a mixture of tert-butyl ((4-carbamimidoyl-5-fluorothiophen-2-yl)methyl)carbamate (120 mg, 0.44 mmol) and di-tert-butyl dicarbonate (287 mg, 1.32 mmol) in THE (3 mL) was added a solution of NaHCO₃ (443 mg, 5.27 mmol) in H₂O (1 mL) drop-wise at 0° C. and the mixture was stirred at room temperature for 2 hours. The mixture was diluted with water and extracted with EtOAc twice. The combined organic layers were dried over anhydrous Na₂SO₄, filtered and concentrated under reduced pressure. The residue was purified by flash chromatography on silica gel (DCM:MeOH=30:1) to give tert-butyl ((4-(N-(tert-butoxycarbonyl)carbamimidoyl)-5-fluorothiophen-2-yl)methyl)carbamate (134 mg, yield 81.7%) as a yellow solid. ¹HNMR (400 MHz, CDCl₃) δ 7.19 (d, J=3.6 Hz, 1H), 4.28 (d, J=4.8 Hz, 2H), 1.67-1.52 (m, 18H). LC/MS (ESI) m/z: 374 (M+H)⁺.

### Step 15: tert-butyl ((5-(aminomethyl)-2-fluorothiophen-3-yl)(imino)methyl)carbamate hydrochloride (16)

A solution of tert-butyl ((4-(N-(tert-butoxycarbonyl)carbamimidoyl)-5-fluorothiophen-2-yl)methyl)carbamate (40 mg, 0.11 mmol) in HCl/1,4-dioxane (2 mL) was stirred at room temperature for 2 hours. The mixture was concentrated to dryness under reduced pressure to give tert-butyl ((5-(aminomethyl)-2-fluorothiophen-3-yl)(imino)methyl)carbamate hydrochloride (29 mg, yield 85.0%) as a white solid, which was directly used in the next reaction without further purification. LC/MS (ESI) m/z: 274 (M+H)⁺.

### Step 16: tert-butyl ((2-fluoro-5-(((1S,3S,5S)-5-methyl-2-((4-phenoxybenzoyl)glycyl)-2-azabicyclo[3.1.0]hexane-3-carboxamido)methyl)thiophen-3-yl)(imino)methyl)carbamate (17)

To a mixture of tert-butyl ((5-(aminomethyl)-2-fluorothiophen-3-yl)(imino)methyl)carbamate hydrochloride (29 mg, 0.11 mmol) and (1 S,3S,5S)-5-methyl-2-{2-[(4-phenoxyphenyl) formamido]acetyl}-2-azabicyclo[3.1.0]hexane-3-carboxylic acid (46 mg, 0.12 mmol) in DMF (2 mL) was added DIPEA (0.09 mL, 0.53 mmol) and PyBOP (83 mg, 0.16 mmol) at 0° C. under N₂ atmosphere and the mixture was stirred at room temperature for 0.5 hours. The mixture was quenched with water and extracted with EtOAc twice. The combined organic layers were washed with brine, dried over anhydrous Na₂SO₄, filtered and concentrated to dryness under reduced pressure. The residue was purified by prep-TLC (DCM:MeOH=20:1) to give tert-butyl N-[2-fluoro-5-({[(1 S,3S,5S)-5-methyl-2-{2-[(4-phenoxyphenyl)formamido]acetyl}-2-azabicyclo[3.1.0]hexan-3-yl]formamido}methyl)thiophene-3-carboximidoyl]carbamate (35 mg, yield 50.8%) as a colorless oil. LC/MS (ESI) m/z: 650 (M+H)⁺.

### Step 17: (1S,3S,5S)-N-((4-carbamimidoyl-5-fluorothiophen-2-yl)methyl)-5-methyl-2-((4-phenoxybenzoyl)glycyl)-2-azabicyclo[3.1.0]hexane-3-carboxamide (Compound 239)

To a solution of tert-butyl N-[2-fluoro-5-({[(1 S,3S,5S)-5-methyl-2-{2-[(4-phenoxyphenyl)formamido]acetyl}-2-azabicyclo[3.1.0]hexan-3-yl]formamido}methyl) thiophene-3-carboximidoyl]carbamate (35 mg, 0.054 mmol) in DCM (2 mL) was added TFA (1 mL) at 0° C. under N₂ atmosphere. The reaction mixture was stirred at room temperature for 2 hours. The mixture was concentrated under reduced pressure to dryness. The residue was purified by prep-HPLC to give Compound 239 (3.3 mg, yield 11.1%) as a yellow solid. ¹H NMR (400 MHz, CD₃OD) δ 7.86 (d, J=8.8 Hz, 2H), 7.42 (t, J=8.0 Hz, 2H), 7.21 (t, J=7.4 Hz, 1H), 7.07 (d, J=7.6 Hz, 2H), 7.03-6.97 (m, 3H), 4.82 (dd, J=11.2, 11.2 Hz, 1H), 4.48-4.27 (m, 4H), 3.49-3.40 (m, 1H), 2.54-2.36 (m, 1H), 2.21-2.03 (m, 1H), 1.30 (s, 3H), 1.12 (dd, J=5.6, 5.6 Hz, 1H), 0.81 (t, J=5.6 Hz, 1H). LC/MS (ESI) m/z: 550(M+H)⁺.

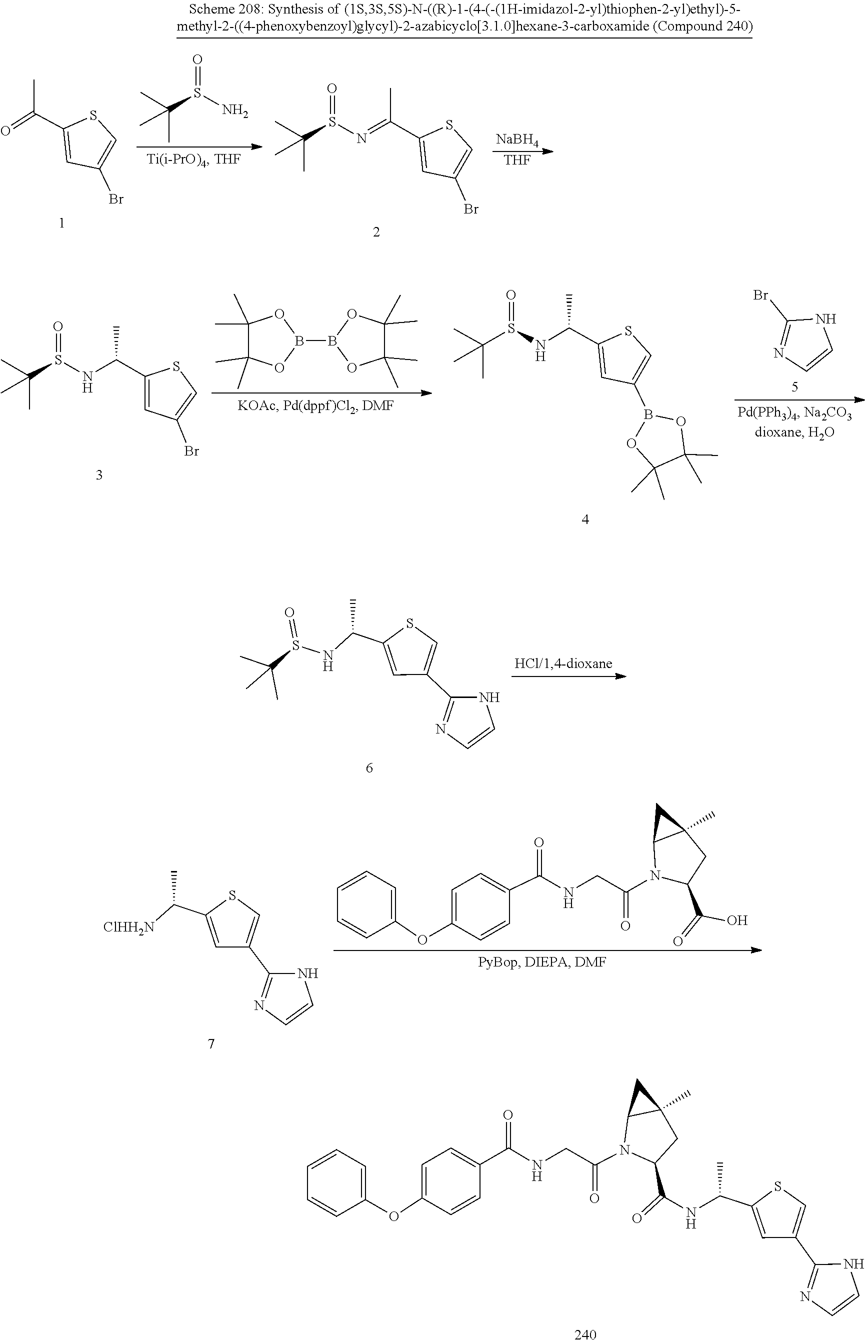

### Step 1: (R,E)-N-(1-(4-bromothiophen-2-yl)ethylidene)-2-methylpropane-2-sulfinamide (2)

To a mixture of 1-(4-bromothiophen-2-yl)ethan-1-one (15 g, 73.1 mmol) in THE (200 mL) was added Ti(i-PrO)₄ (103.9 g, 365.5 mmol) and (R)-2-methylpropane-2-sulfinamide (22.15 g, 182.8 mmol) and the mixture was stirred at 90° C. overnight. The mixture was diluted with water and extracted with EtOAc twice, the combined organic layers were washed with water and brine, dried over anhydrous Na₂SO₄, filtered and concentrated under reduced pressure to dryness. The residue was purified by flash chromatography (PE:EtOAc=3:1) to give (R,E)-N-(1-(4-bromothiophen-2-yl)ethylidene)-2-methylpropane-2-sulfinamide (17 g, yield 75.4%) as yellow solid. LC/MS (ESI) m/z: 308 (M+H)⁺.

### Step 2: (R)-N-((R)-1-(4-bromothiophen-2-yl)ethyl)-2-methylpropane-2-sulfinamide (3)

To a solution of (R,E)-N-(1-(4-bromothiophen-2-yl)ethylidene)-2-methylpropane-2-sulfinamide (17.0 g, 55.2 mmol) in THE (200 mL) was added NaBH₄ (6.29 g, 165.5 mmol) in portions at −30° C. The mixture was stirred at −30° C. under N₂ atmosphere for 4 hours. The mixture was diluted with water and extracted with EtOAc twice. The combined organic layers were washed with brine, dried over anhydrous Na₂SO₄, filtered and concentrated to dryness under reduced pressure. The residue was purified by flash chromatography on silica gel (PE:EtOAc=3:1) to give a mixture. The mixture was recrystallization with EtOAc to afford (R)-N-(1-(4-bromothiophen-2-yl)ethyl)-2-methylpropane-2-sulfinamide (8.1 g, yield 47.3%) as white solid and the absolute configuration was determined by Mosher's method. LC/MS (ESI) m/z: 310 (M+H)⁺.

### Step 3: (R)-2-methyl-N-((R)-1-(4-(4,4,5,5-tetramethyl-1,3,2-dioxaborolan-2-yl)thiophen-2-yl)ethyl)propane-2-sulfinamide (4)

To a solution of (R)-N-((R)-1-(4-bromothiophen-2-yl)ethyl)-2-methylpropane-2-sulfinamide (250 mg, 0.59 mmol) in DMF (15 mL) was added 4,4,4′,4′,5,5,5′,5′-octamethyl-2,2′-bi(1,3,2-dioxaborolane) (300 mg, 1.18 mmol), Pd(dppf)Cl₂ (25 mg, 0.03 mmol) and KOAc (170 mg, 1.8 mmol) under N₂ atmosphere and the reaction mixture was stirred at 90° C. overnight. The reaction solution was used directly in next step without further purification. LC/MS (ESI) m/z: 358 (M+H)⁺.

### Step 4: (R)-N-((R)-1-(4-(1H-imidazol-2-yl)thiophen-2-yl)ethyl)-2-methylpropane-2-sulfinamide (6)

To a solution of (R)-2-methyl-N-((R)-1-(4-(4,4,5,5-tetramethyl-1,3,2-dioxaborolan-2-yl)thiophen-2-yl)ethyl)propane-2-sulfinamide (0.59 mmol, 15 mL) in 1,4-dioxane/H₂O (20 mL, 4/1) was added 2-bromo-1H-imidazole (173 mg, 1.18 mmol), Na₂CO₃ (125 mg, 1.18 mmol) and Pd(PPh₃)₄ (25 mg, 0.02 mmol) at room temperature, and the reaction mixture was stirred under N₂ atmosphere at 90° C. overnight. The mixture was extracted with EtOAc twice and the combined organic layers were washed with brine, dried over anhydrous Na₂SO₄, filtered and concentrated under reduced pressure to dryness. The residue was purified by flash chromatography on silica gel (PE:EtOAc=1:1) to give (R)-N-((R)-1-(4-(1H-imidazol-2-yl)thiophen-2-yl)ethyl)-2-methylpropane-2-sulfinamide (132 mg, 2 steps yield 75.2%) as a yellow solid. LC/MS (ESI) m/z: 298 (M+H)⁺.

### Step 5: (R)-1-(4-(1H-imidazol-2-yl)thiophen-2-yl)ethan-1-amine hydrochloride (9)

To a solution of (R)-N-((R)-1-(4-(1H-imidazol-2-yl)thiophen-2-yl)ethyl)-2-methylpropane-2-sulfinamide (132 mg, 0.44 mmol) in DCM (2.0 mL) was added HCl/1,4-dioxane (2.0 mL) at room temperature and the mixture was stirred for 2 hours. The reaction mixture was concentrated under reduced pressure to give (R)-1-(4-(1H-imidazol-2-yl)thiophen-2-yl)ethan-1-amine hydrochloride (102 mg, 100% yield) as a brown solid, which was used directly in next step without further purification. LC/MS (ESI) (m/z): 177 (M−NH₂)⁺.

### Step 6: (1S,3S,5S)-N-((R)-1-(4-(1H-imidazol-2-yl)thiophen-2-yl)ethyl)-5-methyl-2-((4-phenoxybenzoyl)glycyl)-2-azabicyclo[3.1.0]hexane-3-carboxamide (Compound 240)

To a mixture of (1 S,3S,5S)-5-methyl-2-((4-phenoxybenzoyl)glycyl)-2-azabicyclo[3.1.0]hexane-3-carboxylic acid (32 mg, 0.08 mmol), (R)-1-(4-(1H-imidazol-2-yl)thiophen-2-yl)ethan-1-amine hydrochloride (34 mg, 0.15 mmol) in DMF (0.5 mL) was added DIPEA (36 mg, 0.28 mmol) and PyBop (52 mg, 0.1 mmol) at 0° C., and the reaction mixture was stirred at room temperature for 1 hour. The mixture was diluted with ethyl acetate and washed with saturated aq.NaHCO₃ solution. The combined organic layers were washed with brine, dried over anhydrous Na₂SO₄, filtered and concentrated under reduced pressure to dryness. The residue was purified by prep-HPLC to afford Compound 240 (7.2 mg, yield 15.6%) as a white solid. ¹H NMR (400 MHz, CD₃OD) δ 8.34 (s, 1H), 7.81 (dd, J=11.4, 8.8 Hz, 2H), 7.69 (s, 1H), 7.50 (s, 1H), 7.42 (dd, J=8.4, 7.6 Hz, 2H), 7.21 (t, J=7.4 Hz, 1H), 7.06 (dd, J=7.7, 6.7 Hz, 2H), 7.01 (s, 2H), 6.93 (d, J=8.8 Hz, 2H), 5.33-5.20 (m, 1H), 4.84 (d, J=3.4 Hz, 1H), 4.41 (d, J=16.5 Hz, 1H), 4.22 (d, J=16.5 Hz, 1H), 3.38 (dd, J=5.9, 2.4 Hz, 1H), 2.43 (t, J=11.9 Hz, 1H), 2.17 (dd, J=13.4, 3.4 Hz, 1H), 1.59 (d, J=6.9 Hz, 3H), 1.29 (s, 3H), 1.17 (dd, J=5.7, 2.4 Hz, 1H), 0.73 (t, J=5.4 Hz, 1H). LC/MS (ESI) m/z: 570 (M+H)⁺.

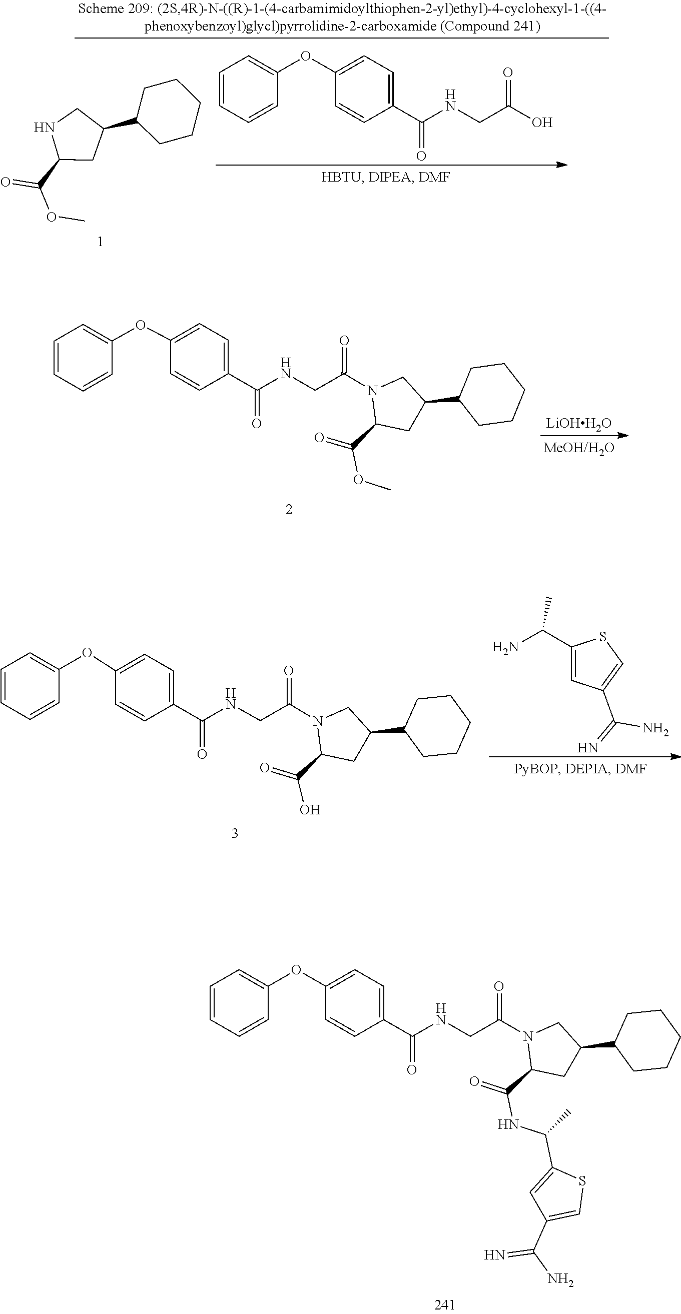

### Step 1: methyl (2S,4R)-4-cyclohexyl-1-((4-phenoxybenzoyl)glycyl)pyrrolidine-2-carboxylate

To a mixture of methyl (2S,4R)-4-cyclohexylpyrrolidine-2-carboxylate (40 mg, 0.19 mmol) and (4-phenoxybenzoyl)glycine (51 mg, 0.19 mmol) in DMF (2 mL) was added DIPEA (147 mg, 1.1 mmol) and HBTU (106 mg, 0.28 mmol) at 0° C. under N₂ atmosphere and the mixture was stirred at 25° C. for 2 hours. The mixture was diluted with water and extracted with EtOAc twice. The combined organic layers were dried over anhydrous Na₂SO₄, filtered and concentrated under reduced pressure to dryness. The residue was purified by column chromatography on silica gel (PE:EtOAc=2:1) to give methyl (2S,4R)-4-cyclohexyl-1-((4-phenoxybenzoyl)glycyl)pyrrolidine-2-carboxylate (58 mg, yield 65.8%) as colorless oil. LC/MS (ESI) m/z: 465 (M+H)⁺.

### Step 2: (2S,4R)-4-cyclohexyl-1-((4-phenoxybenzoyl)glycyl)pyrrolidine-2-carboxylic acid

To a solution of methyl (2S,4R)-4-cyclohexyl-1-((4-phenoxybenzoyl)glycyl)pyrrolidine-2-carboxylate (58 mg, 0.12 mmol) in MeOH (2 mL) and water (1 mL) was added LiOH·H₂O (30 mg, 0.75 mmol) at 0° C. and the mixture was stirred at room temperature for 2 hours. The mixture was acidified with 1N aq.HCl to pH-3, extracted with EtOAc twice. The combined organic layers were washed with brine, dried over anhydrous Na₂SO₄, filtered and concentrated under reduced pressure to give (2S,4R)-4-cyclohexyl-1-((4-phenoxybenzoyl) glycyl)pyrrolidine-2-carboxylic acid (50 mg, yield 92.6%) as colorless oil, which was used directly in the next reaction. LC/MS (ESI) (m/z): 451 (M+H)⁺.

### Step 3: (2S,4R)-N-((R)-1-(4-carbamimidoylthiophen-2-yl)ethyl)-4-cyclohexyl-1-((4-phenoxybenzoyl)glycyl)pyrrolidine-2-carboxamide (Compound 241)

To a mixture of (2S,4R)-4-cyclohexyl-1-((4-phenoxybenzoyl)glycyl)pyrrolidine-2-carboxylic acid (50 mg, 0.11 mol) and (R)-5-(1-aminoethyl)thiophene-3-carboximidamide (27 mg, 0.16 mmol) in DMF (2 mL) was added DIPEA (71 mg, 0.55 mmol) and PyBOP (62 mg, 0.12 mmol) at 0° C. under N₂ atmosphere and the mixture was stirred at 25° C. for 2 hours. The mixture was diluted with water and extracted with EtOAc twice. The combined organic layers were dried over anhydrous Na₂SO₄, filtered and concentrated under reduced pressure to dryness. The residue was purified by column chromatography on silica gel (DCM:MeOH=8:1) and further purified by prep-HPLC to give Compound 241 (1.5 mg, yield 2.3%) as a colorless oil. ¹H NMR (400 MHz, CD₃OD) δ 8.53 (s, 1H), 8.24 (t, J=3.7 Hz, 1H), 7.86 (d, J=8.8 Hz, 2H), 7.49 (d, J=15.9 Hz, 1H), 7.42 (t, J=8.0 Hz, 2H), 7.21 (t, J=7.4 Hz, 1H), 7.06 (d, J=7.8 Hz, 2H), 7.01 (d, J=8.7 Hz, 2H), 5.34-5.24 (m, 1H), 4.58 (s, 3H), 4.20 (t, J=11.0 Hz, 2H), 3.94 (t, J=8.4 Hz, 1H), 2.23-2.14 (m, 2H), 2.04 (s, 1H), 1.94-1.88 (m, 1H), 1.75 (d, J=12.4 Hz, 3H), 1.57 (d, J=7.0 Hz, 3H), 1.30 (s, 5H), 1.03 (d, J=11.5 Hz, 1H). LC/MS (ESI) (m/z): 554 (M+H)⁺.

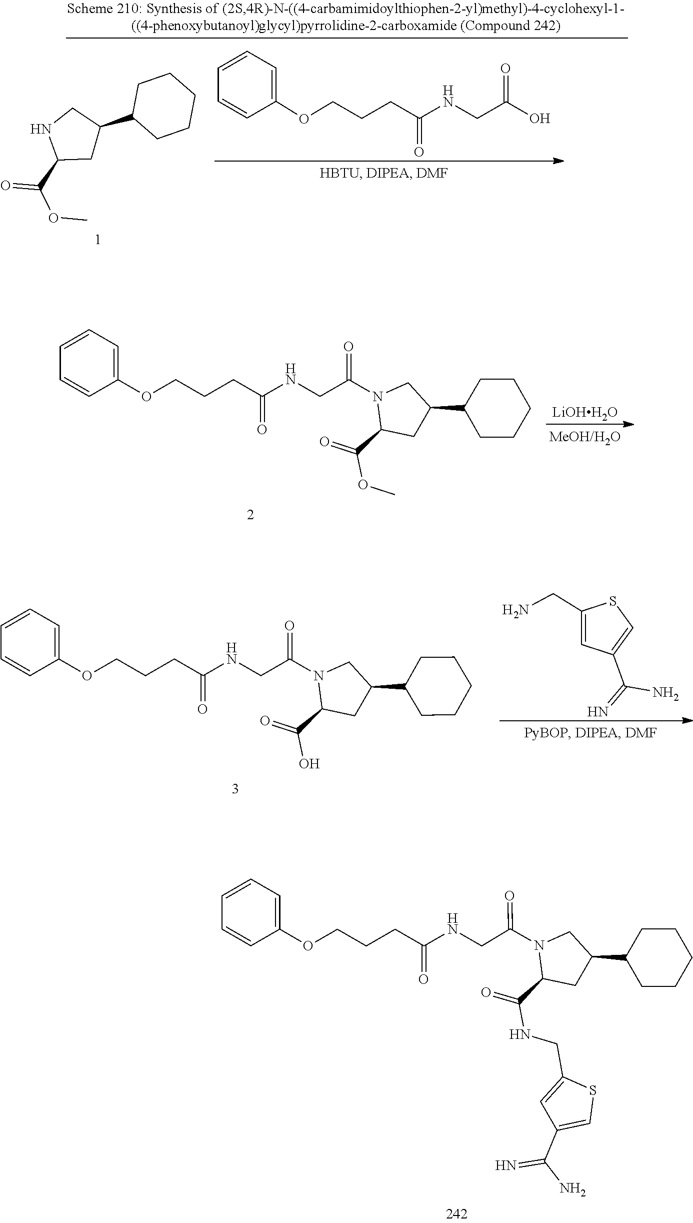

### Step 1: methyl (2S,4R)-4-cyclohexyl-1-((4-phenoxybutanoyl)glycyl)pyrrolidine-2-carboxylate

To a mixture of methyl (2S,4R)-4-cyclohexylpyrrolidine-2-carboxylate (90 mg, 0.38 mmol) and (4-phenoxybutanoyl)glycine (80 mg, 0.38 mmol) in DMF (2 mL) was added DIPEA (294 mg, 2.3 mmol) and HBTU (216 mg, 0.57 mmol) at 0° C. under N₂ atmosphere and the mixture was stirred at 25° C. for 2 hours. The mixture was diluted with water, extracted with EtOAc twice. The combined organic layers were dried over anhydrous Na₂SO₄, filtered and concentrated to dryness under reduced pressure. The residue was purified by column chromatography on silica gel (PE:EtOAc=2:1) to give methyl (2S,4R)-4-cyclohexyl-1-((4-phenoxybutanoyl)glycyl)pyrrolidine-2-carboxylate (40 mg, yield 24.5%) as a colorless oil. LC/MS (ESI) m/z: 431 (M+H)⁺.

### Step 2: (2S,4R)-4-cyclohexyl-1-((4-phenoxybutanoyl)glycyl)pyrrolidine-2-carboxylic acid

To a solution of methyl (2S,4R)-4-cyclohexyl-1-((4-phenoxybutanoyl)glycyl)pyrrolidine-2-carboxylate (40 mg, 93 umol) in MeOH (2 mL) and water (1 mL) was added LiOH·H₂O (22 mg, 0.56 mmol) at 0° C. and the mixture was stirred at room temperature for 2 hours. The mixture was acidified with 1N aq.HCl to pH-3 and extracted with EtOAc twice. The combined organic layers were washed with brine, dried over anhydrous Na₂SO₄, filtered and concentrated under reduced pressure to give (2S,4R)-4-cyclohexyl-1-((4-phenoxybutanoyl) glycyl)pyrrolidine-2-carboxylic acid (30 mg, yield 77.5%) as a colorless oil, which was used directly in the next reaction. LC/MS (ESI) (m/z): 417 (M+H)⁺.

### Step 3: (2S,4R)-N-((4-carbamimidoylthiophen-2-yl)methyl)-4-cyclohexyl-1-((4-phenoxybutanoyl)glycyl)pyrrolidine-2-carboxamide (Compound 242)

To a mixture of (2S,4R)-4-cyclohexyl-1-((4-phenoxybutanoyl)glycyl)pyrrolidine-2-carboxylic acid (15 mg, 36 umol) and 5-(aminomethyl)thiophene-3-carboximidamide (10 mg, 54 umol) in DMF (2 mL) was added DIPEA (23 mg, 0.18 mmol) and PyBOP (21 mg, 40 umol) at 0° C. under N₂ atmosphere and the mixture was stirred at 25° C. for 2 hours. The mixture was diluted with water and extracted with EtOAc twice. The combined organic layers were dried over anhydrous Na₂SO₄, filtered and concentrated under reduced pressure to dryness. The residue was purified by column chromatography on silica gel (DCM:MeOH=8:1) and prep-HPLC to give Compound 242 (2.0 mg, yield 10.0%) as a colorless oil. ¹H NMR (400 MHz, CD₃OD) δ 8.52 (s, 1H), 8.22 (t, J=3.4 Hz, 1H), 7.43 (d, J=28.4 Hz, 1H), 7.24 (t, J=7.9 Hz, 2H), 6.90 (dd, J=7.3, 5.2 Hz, 3H), 4.60-4.50 (m, 4H), 4.09 (d, J=16.9 Hz, 1H), 3.99 (dd, J=10.6, 4.2 Hz, 3H), 3.89-3.83 (m, 1H), 3.22 (t, J=9.8 Hz, 1H), 2.46 (t, J=7.4 Hz, 2H), 2.16-2.04 (m, 4H), 1.73 (d, J=13.0 Hz, 6H), 1.25 (d, J=11.5 Hz, 2H), 1.10-0.82 (m, 3H). LC/MS (ESI) (m/z): 554 (M+H)⁺.

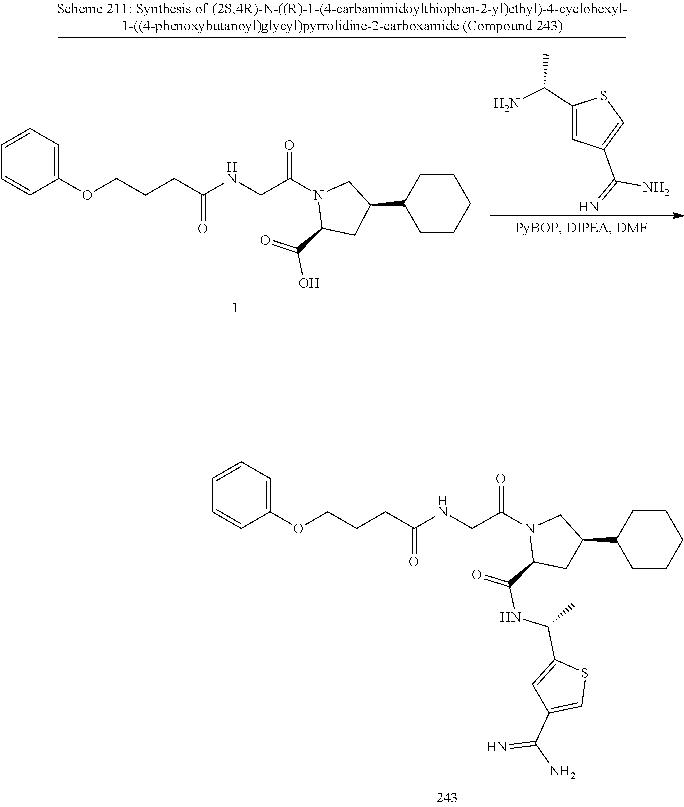 [SMILES: O=C(CCCOc1ccccc1)NCC(=O)N1C[C@@H](C2CCCCC2)C[C@H]1C(=O)O ; C[C@@H](N)c1cc(C(=N)N)cs1 ; C[C@@H](NC(=O)[C@@H]1C[C@H](C2CCCCC2)CN1C(=O)CNC(=O)CCCOc1ccccc1)c1cc(C(=N)N)cs1]

### Step 1: (2S,4R)-N-((R)-1-(4-carbamimidoylthiophen-2-yl)ethyl)-4-cyclohexyl-1-((4-phenoxybutanoyl)glycyl)pyrrolidine-2-carboxamide (Compound 243)

To a mixture of (2S,4R)-4-cyclohexyl-1-((4-phenoxybutanoyl)glycyl)pyrrolidine-2-carboxylic acid (15 mg, 36 umol) and (R)-5-(1-aminoethyl)thiophene-3-carboximidamide (24 mg, 72 umol) in DMF (2 mL) was added DIPEA (28 mg, 0.22 mmol) and PyBOP (24 mg, 47 umol) at 0° C. under N₂ atmosphere and the mixture was stirred at 25° C. for 2 hours. The mixture was diluted with water and extracted with EtOAc twice. The combined organic layers were dried over anhydrous Na₂SO₄, filtered and concentrated under reduced pressure to dryness. The residue was purified by column chromatography on silica gel (DCM:MeOH=8:1) and further purified by prep-HPLC to give Compound 243 (1.6 mg, yield 7.8%) as a white solid. ¹H NMR (400 MHz, CD₃OD) δ 8.23 (d, J=1.4 Hz, 1H), 7.47 (s, 1H), 7.23 (t, J=8.0 Hz, 2H), 6.91-6.87 (m, 3H), 5.31-5.22 (m, 1H), 4.61-4.47 (m, 4H), 4.06-3.94 (m, 4H), 3.86 (dd, J=17.2, 8.8 Hz, 1H), 3.19 (dd, J=26.3, 16.5 Hz, 1H), 2.47 (t, J=7.4 Hz, 2H), 2.15-2.03 (m, 4H), 1.73 (d, J=14.5 Hz, 5H), 1.56 (d, J=7.0 Hz, 3H), 1.30-1.20 (m, 4H). LC/MS (ESI) (m/z): 568 (M+H)⁺.

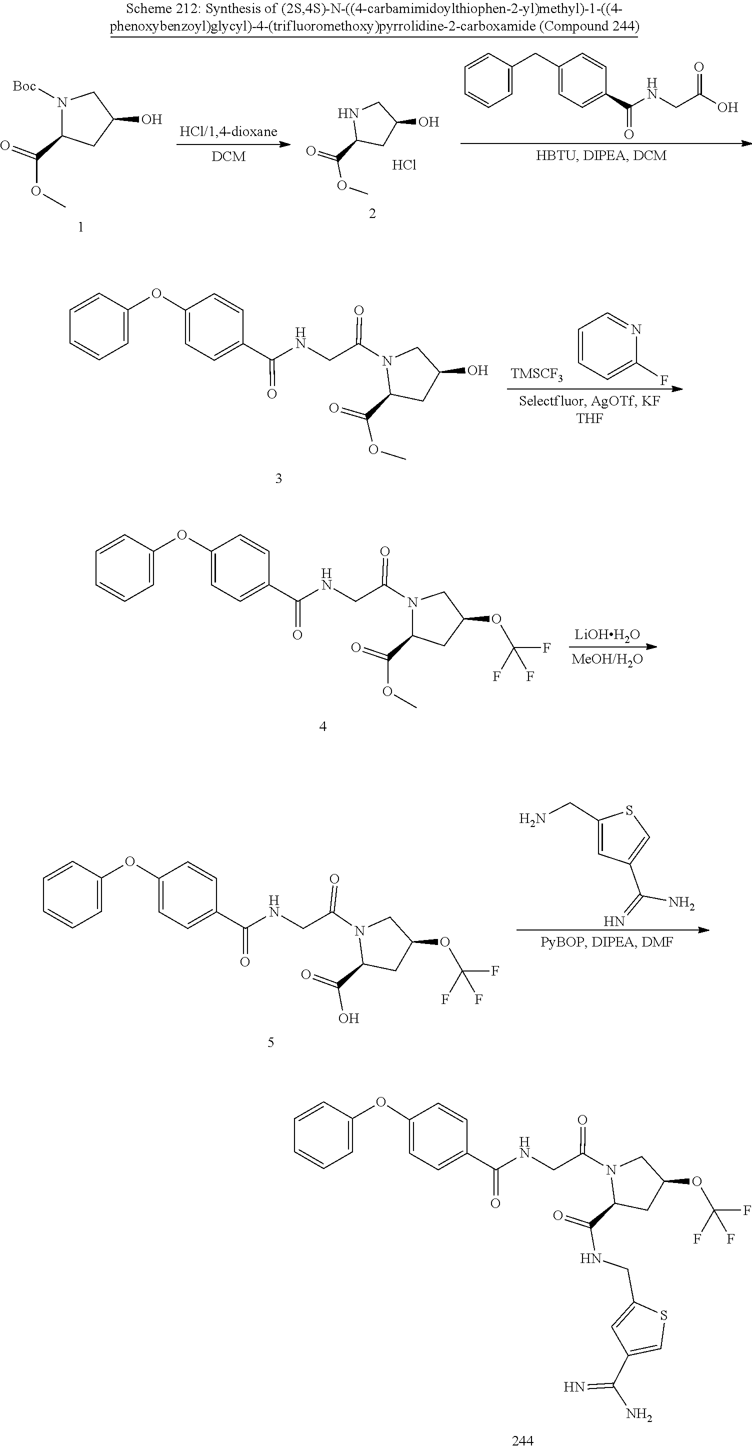

### Step 1: methyl (2S,4S)-4-hydroxypyrrolidine-2-carboxylate hydrochloride

A solution of 1-(tert-butyl) 2-methyl (2S,4S)-4-hydroxypyrrolidine-1,2-dicarboxylate (700 mg, 2.9 mmol) in HCl/1,4-dioxane (10 mL) was stirred under N₂ atmosphere at room temperature for 2 hours. The reaction mixture was concentrated to dryness under reduced pressure to give methyl (2S,4S)-4-hydroxypyrrolidine-2-carboxylate hydrochloride (500 mg, yield 96.4%), which was used directly in the next step without further purification. LC/MS (ESI) m/z: 146 (M+H)⁺.

### Step 2: methyl (2S,4S)-4-hydroxy-1-((4-phenoxybenzoyl)glycyl)pyrrolidine-2-carboxylate

To a mixture of methyl (2S,4S)-4-hydroxypyrrolidine-2-carboxylate hydrochloride (500 mg, 2.8 mmol) and (4-phenoxybenzoyl)glycine (750 mg, 2.8 mmol) in DMF (8 mL) was added DIPEA (2.2 g, 17 mmol) and HBTU (1.6 g, 4.3 mmol) at 0° C. under N₂ atmosphere and the mixture was stirred at 25° C. for 2 hours. The mixture was diluted with EtOAc, washed with water and brine, dried over anhydrous Na₂SO₄, filtered and concentrated under reduced pressure to dryness. The residue was purified by column chromatography on silica gel (PE:EtOAc=2:1) to give methyl (2S,4S)-4-hydroxy-1-((4-phenoxybenzoyl)glycyl)pyrrolidine-2-carboxylate (1.1 g, yield 96.5%) as a colorless oil. LC/MS (ESI) m/z: 399 (M+H)⁺.

### Step 3: methyl (2S,4S)-1-((4-phenoxybenzoyl)glycyl)-4-(trifluoromethoxy)pyrrolidine-2-carboxylate

To a solution of methyl (2S,4S)-4-hydroxy-1-((4-phenoxybenzoyl)glycyl)pyrrolidine-2-carboxylate (1.0 g, 2.5 mmol) in EtOAc (15 mL) was added selectfluor (4.4 g, 12 mmol), AgOTf (6.4 g, 25 mmol), KF (1.5 g, 25 mmol), 2-fluoropyridine (2.4 g, 25 mmol) and TMSCF₃ (3.5 g, 25 mmol) under N₂ atmosphere and the mixture was stirred at 40° C. for 48 hours. The mixture was diluted with EtOAc, washed with water and brine, dried over anhydrous Na₂SO₄, filtered and concentrated under reduced pressure to dryness. The residue was purified by column chromatography on silica gel (PE:EtOAc=2:1) to give methyl (2S,4S)-1-((4-phenoxybenzoyl)glycyl)-4-(trifluoromethoxy) pyrrolidine-2-carboxylate (200 mg, yield 17.1%) as a colorless oil. LCMS (ESI) m/z: 467 (M+H)⁺.

### Step 4: (2S,4S)-1-((4-phenoxybenzoyl)glycyl)-4-(trifluoromethoxy)pyrrolidine-2-carboxylic acid

To a solution of methyl (2S,4S)-1-((4-phenoxybenzoyl)glycyl)-4-(trifluoromethoxy) pyrrolidine-2-carboxylate (100 mg, 0.21 mmol) in MeOH (2 mL) and water (1 mL) was added LiOH·H₂O (33 mg, 0.81 mmol) at 0° C. and the mixture was stirred at room temperature for 2 hours. The mixture was acidified with 1N aq.HCl to pH-3 and extracted with EtOAc twice. The combined organic layers were washed with brine, dried over anhydrous Na₂SO₄, filtered and concentrated to dryness to give (2S,4S)-1-((4-phenoxybenzoyl)glycyl)-4-(trifluoromethoxy)pyrrolidine-2-carboxylic acid (90 mg, yield 93.0%) as a colorless oil, which was used directly in the next reaction. LC/MS (ESI) (m/z): 453 (M+H)⁺.

### Step 5: (2S,4S)-N-((4-carbamimidoylthiophen-2-yl)methyl)-1-((4-phenoxybenzoyl) glycyl)-4-(trifluoromethoxy)pyrrolidine-2-carboxamide (Compound 244)

To a mixture of (2S,4S)-1-((4-phenoxybenzoyl)glycyl)-4-(trifluoromethoxy)pyrrolidine-2-carboxylic acid (90 mg, 0.20 mmol) and 5-(aminomethyl)thiophene-3-carboximidamide (46 mg, 0.30 mmol) in DMF (2 mL) was added DIPEA (155 mg, 1.2 mmol) and PyBOP (114 mg, 0.22 mmol) at 0° C. under N₂ atmosphere and the mixture was stirred at 25° C. for 2 hours. The mixture was diluted with EtOAc, washed with water and brine, dried over anhydrous Na₂SO₄, filtered and concentrated under reduced pressure to dryness. The residue was purified by column chromatography on silica gel (DCM:MeOH=8:1) and further purified by prep-HPLC to give Compound 244 (1.2 mg, yield 1.0%) as a white solid. ¹H NMR (400 MHz, CD₃OD) δ 8.52 (s, 1H), 8.19 (s, 1H), 7.84 (d, J=8.7 Hz, 2H), 7.41 (d, J=8.5 Hz, 3H), 7.21 (s, 1H), 7.06 (d, J=7.8 Hz, 2H), 7.00 (d, J=8.7 Hz, 2H), 5.15 (d, J=3.7 Hz, 1H), 4.66 (dd, J=12.6, 5.4 Hz, 2H), 4.29 (d, J=16.6 Hz, 1H), 4.10-4.05 (m, 2H), 3.99 (d, J=12.0 Hz, 1H), 2.60 (dd, J=11.6, 8.1 Hz, 1H), 2.44 (d, J=13.6 Hz, 1H), 2.03 (d, J=7.0 Hz, 1H). LC/MS (ESI) m/z: 590 (M+H)⁺.

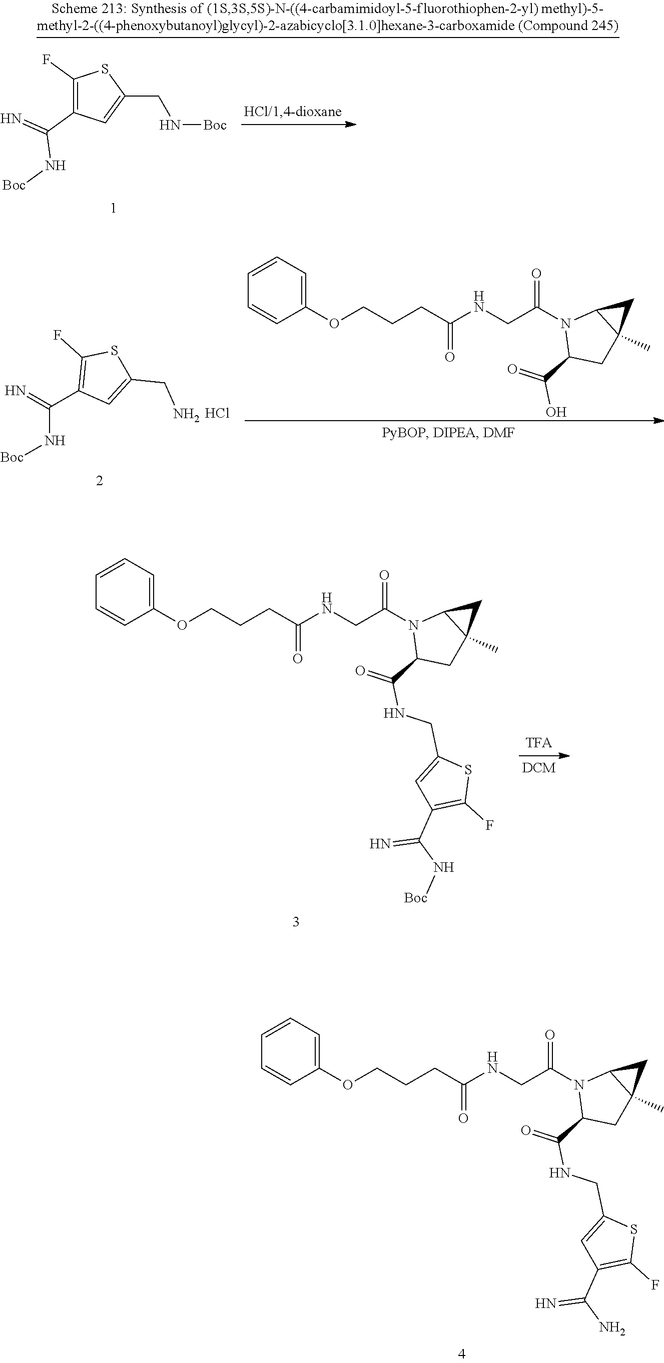 [SMILES: CC(C)(C)OC(=O)NCc1cc(C(=N)NC(=O)OC(C)(C)C)c(F)s1 ; CC(C)(C)OC(=O)NC(=N)c1cc(CN)sc1F ; Cl ; C[C@@]12C[C@@H]1N(C(=O)CNC(=O)CCCOc1ccccc1)[C@H](C(=O)O)C2 ; CC(C)(C)OC(=O)NC(=N)c1cc(CNC(=O)[C@@H]2C[C@]3(C)C[C@@H]3N2C(=O)CNC(=O)CCCOc2ccccc2)sc1F ; C[C@@]12C[C@@H]1N(C(=O)CNC(=O)CCCOc1ccccc1)[C@H](C(=O)NCc1cc(C(=N)N)c(F)s1)C2]

### Step 1: tert-butyl ((5-(aminomethyl)-2-fluorothiophen-3-yl)(imino)methyl)carbamate hydrochloride (2)

A solution of tert-butyl ((4-(N-(tert-butoxycarbonyl)carbamimidoyl)-5-fluorothiophen-2-yl) methyl)carbamate (45 mg, 0.12 mmol) in HCl/1,4-dioxane (2 mL) was stirred at room temperature for 1 hour. The mixture was concentrated to dryness under reduced pressure to give tert-butyl ((5-(aminomethyl)-2-fluorothiophen-3-yl)(imino)methyl)carbamate hydrochloride (32 mg, yield 97.2%) as a white solid, which was directly used in the next reaction without further purification. LC/MS (ESI) m/z: 274 (M+H)⁺.

### Step 2: tert-butyl ((2-fluoro-5-(((1S,3S,5S)-5-methyl-2-((4-phenoxybutanoyl)glycyl)-2-azabicyclo[3.1.0]hexane-3-carboxamido)methyl)thiophen-3-yl)(imino)methyl)carbamate (3)

To a mixture of tert-butyl ((5-(aminomethyl)-2-fluorothiophen-3-yl)(imino)methyl)carbamate hydrochloride (32 mg, 0.10 mmol) and (1 S,3S,5S)-5-methyl-2-((4-phenoxybutanoyl)glycyl)-2-azabicyclo[3.1.0]hexane-3-carboxylic acid (43 mg, 0.12 mmol) in DMF (2 mL) was added DIPEA (0.08 mL, 0.50 mmol) and PyBOP (78 mg, 0.15 mmol) at 0° C. under N₂ atmosphere and the mixture was stirred at room temperature for 0.5 hours. The mixture was quenched with water and extracted with EtOAc twice. The combined organic layers were washed with brine, dried over anhydrous Na₂SO₄, filtered and concentrated under reduced pressure to dryness. The residue was purified by pre-TLC (DCM:MeOH=20:1) to give tert-butyl ((2-fluoro-5-(((1S,3S,5S)-5-methyl-2-((4-phenoxybutanoyl)glycyl)-2-azabicyclo[3.1.0]hexane-3-carboxamido)methyl)thiophen-3-yl)(imino)methyl)carbamate (30 mg, yield 48.8%) as a colorless oil. LC/MS (ESI) m/z: 616 (M+H)⁺.

### Step 3: (1S,3S,5S)-N-((4-carbamimidoyl-5-fluorothiophen-2-yl)methyl)-5-methyl-2-((4-phenoxybutanoyl)glycyl)-2-azabicyclo[3.1.0]hexane-3-carboxamide (Compound 245)

To a solution of tert-butyl ((2-fluoro-5-(((1S,3S,5S)-5-methyl-2-((4-phenoxybutanoyl)glycyl)-2-azabicyclo[3.1.0]hexane-3-carboxamido)methyl)thiophen-3-yl)(imino)methyl)carbamate (30 mg, 0.049 mmol) in DCM (2 mL) was added TFA (1 mL) at 0° C. under N₂ atmosphere. The reaction mixture was stirred at room temperature for 2 hours. The mixture was concentrated under reduced pressure to dryness. The residue was purified by prep-HPLC to give Compound 245 (2.3 mg, yield 9.2%) as a white solid. ¹H NMR (400 MHz, CD₃OD) δ 7.24 (t, J=8.0 Hz, 2H), 6.97 (d, J=2.8 Hz, 1H), 6.93-6.87 (m, 3H), 4.80-4.77 (m, 1H), 4.45-4.33 (m, 2H), 4.24-4.08 (m, 2H), 4.01 (t, J=6.2 Hz, 2H), 3.35-3.33 (m, 1H), 2.48 (t, J=7.6 Hz, 2H), 2.43-2.33 (m, 1H), 2.17-2.05 (m, 3H), 1.28 (s, 3H), 1.11 (dd, J=5.6, 5.6 Hz, 1H), 0.81 (t, J=5.8 Hz, 1H). LC/MS (ESI) m/z: 516 (M+H)⁺.

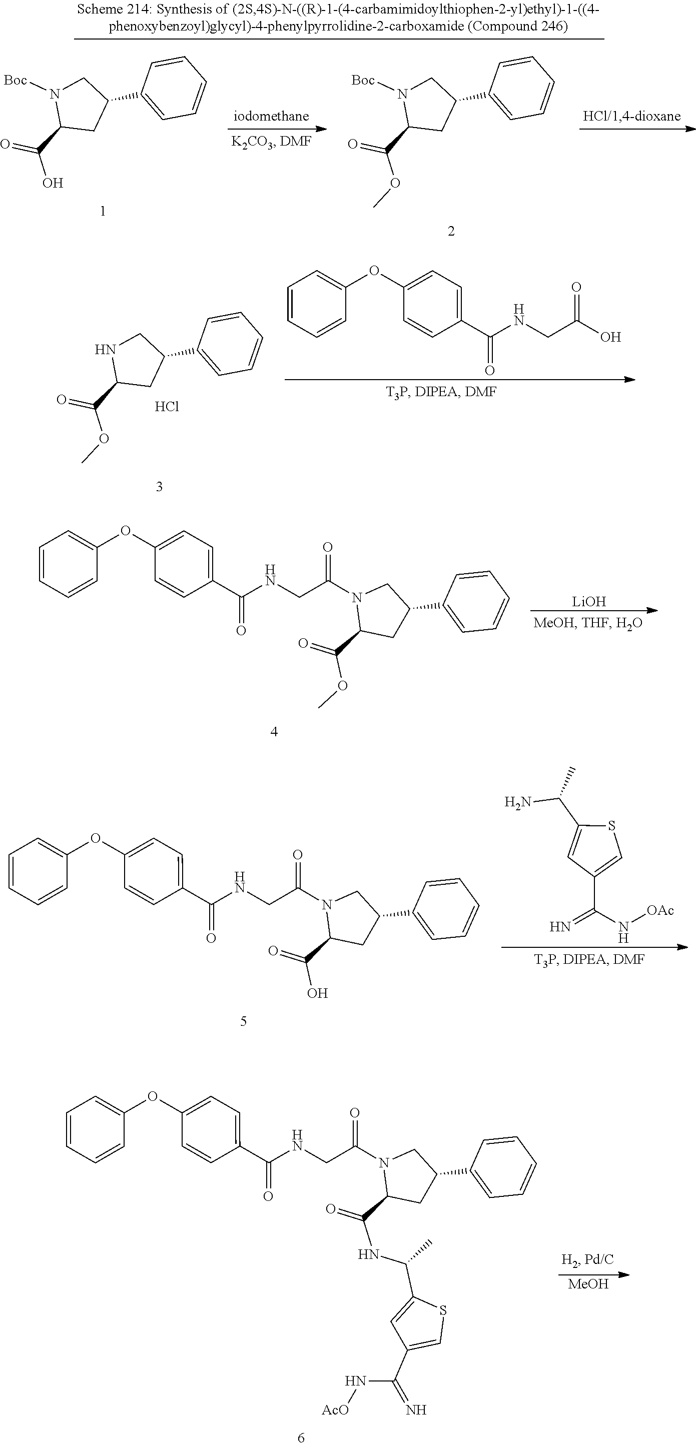

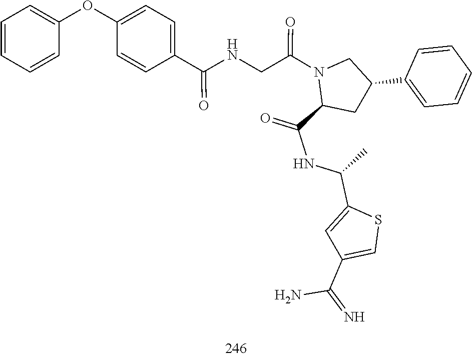 [SMILES: C[C@@H](NC(=O)[C@@H]1C[C@@H](c2ccccc2)CN1C(=O)CNC(=O)c1ccc(Oc2ccccc2)cc1)c1cc(C(=N)N)cs1]

### Step 1: 1-(tert-butyl) 2-methyl (2S,4S)-4-phenylpyrrolidine-1,2-dicarboxylate (2)

To a mixture of (2S,4S)-1-(tert-butoxycarbonyl)-4-phenylpyrrolidine-2-carboxylic acid (120 mg, 0.41 mmol) and iodomethane (175 mg, 1.24 mmol) in DMF (2 mL) was added K₂CO₃ (85 mg, 0.62 mmol) at 0° C. and the mixture was stirred at room temperature for 1 hour. The mixture was diluted EtOAc, washed with water and brine, dried over anhydrous Na₂SO₄, filtered and concentrated under reduced pressure to dryness. The residue was purified by chromatography on silica gel (PE:EtOAc=3:1) to give 1-(tert-butyl) 2-methyl (2S,4S)-4-phenylpyrrolidine-1,2-dicarboxylate (94 mg, yield 74.7%) as a colorless oil. LC/MS (ESI) m/z: 306 (M+H)⁺.

### Step 2: methyl (2S,4S)-4-phenylpyrrolidine-2-carboxylate hydrochloride (3)

A solution of 1-(tert-butyl) 2-methyl (2S,4S)-4-phenylpyrrolidine-1,2-dicarboxylate (90 mg, 0.30 mmol) in HCl/1,4-dioxane (3 mL) was stirred at room temperature for 2 hours. The mixture was concentrated to dryness under reduced pressure to give methyl (2S,4S)-4-phenylpyrrolidine-2-carboxylate hydrochloride (50 mg, yield 63.6%) as a white solid, which was directly used in the next reaction without further purification. LC/MS (ESI) m/z: 206 (M+H)⁺.

### Step 3: methyl (2S,4S)-1-((4-phenoxybenzoyl)glycyl)-4-phenylpyrrolidine-2-carboxylate (4)

To a mixture of methyl (2S,4S)-4-phenylpyrrolidine-2-carboxylate hydrochloride (50 mg, 0.24 mmol) and ((4-phenoxybenzoyl)glycine (53 mg, 0.20 mmol) in DMF (2 mL) was added DIPEA (0.21 mL, 1.22 mmol) and T₃P (232 mg, 0.37 mmol, 50% wt. in EtOAc) at 0° C. under N₂ atmosphere and the mixture was stirred at room temperature for 2 hours. The mixture was quenched with water and extracted with EtOAc twice. The combined organic layers were washed with brine, dried over anhydrous Na₂SO₄, filtered and concentrated to dryness under reduced pressure. The residue was purified by chromatography on silica gel (PE:EtOAc=1:1) to give methyl (2S,4S)-1-((4-phenoxybenzoyl)glycyl)-4-phenylpyrrolidine-2-carboxylate (80 mg, yield 89.5%) as a colorless oil. LC/MS (ESI) m/z: 459 (M+H)⁺.

### Step 4: (2S,4S)-1-((4-phenoxybenzoyl)glycyl)-4-phenylpyrrolidine-2-carboxylic acid (5)

To a solution of methyl (2S,4S)-1-((4-phenoxybenzoyl)glycyl)-4-phenylpyrrolidine-2-carboxylate (80 mg, 0.17 mmol) in MeOH (2 mL) and THE (1 mL) was added a solution of LiOH·H₂O (22 mg, 0.52 mmol) in water (1 mL) at 0° C., and the mixture was stirred at room temperature for 2 hours. The mixture was diluted with water and washed with MTBE twice. The aqueous layer was acidified with 0.5N aq.HCl to pH-3 and extracted with EtOAc twice. The combined organic layers were washed with brine, dried over anhydrous Na₂SO₄, filtered and concentrated under reduced pressure to dryness to give (2S,4S)-1-((4-phenoxybenzoyl)glycyl)-4-phenylpyrrolidine-2-carboxylic acid (139 mg, yield 82.0%) as a colorless oil. LC/MS (ESI) (m/z): 445 (M+H)⁺.

### Step 5: (2S,4S)-N-((R)-1-(4-(N-acetoxycarbamimidoyl)thiophen-2-yl)ethyl)-1-((4-phenoxybenzoyl)glycyl)-4-phenylpyrrolidine-2-carboxamide (6)

To a mixture of (2S,4S)-1-((4-phenoxybenzoyl)glycyl)-4-phenylpyrrolidine-2-carboxylic acid (30 mg, 0.07 mmol) and (R)-N-acetoxy-5-(1-aminoethyl)thiophene-3-carboximidamide (31 mg, 0.14 mmol) in DMF (3 mL) was added DIPEA (0.06 mL, 0.03 mmol) and T₃P (64 mg, 0.10 mmol, 50% wt. in EtOAc) at 0° C. under N₂ atmosphere and the mixture was stirred at 25° C. for 1 hour. The mixture was diluted with EtOAc, washed with saturated aq.NaHCO₃ solution and brine, dried over anhydrous Na₂SO₄, filtered and concentrated under reduced pressure to dryness. The residue was purified by prep-TLC (DCM:MeOH=20:1) to give (2S,4S)-N-((R)-1-(4-(N-acetoxycarbamimidoyl)thiophen-2-yl)ethyl)-1-((4-phenoxybenzoyl)glycyl)-4-phenylpyrrolidine-2-carboxamide (20 mg, yield 45.3%) as a colorless oil. LC/MS (ESI) m/z: 654 (M+H)⁺.

### Step 6: (2S,4S)-N-((R)-1-(4-carbamimidoylthiophen-2-yl)ethyl)-1-((4-phenoxybenzoyl)glycyl)-4-phenylpyrrolidine-2-carboxamide (Compound 246)

To a mixture of (2S,4S)-N-((R)-1-(4-(N-acetoxycarbamimidoyl)thiophen-2-yl)ethyl)-1-((4-phenoxybenzoyl)glycyl)-4-phenylpyrrolidine-2-carboxamide (20 mg, 0.03 mmol) in MeOH (3 mL) was added Pd/C (5 mg, 10% wt.), and the mixture was degassed under N₂ atmosphere for three times and stirred under a H₂ balloon at 25° C. for 2 hours. The mixture was filtered and the filtrate was concentrated under reduced pressure to dryness. The residue was purified by prep-HPLC to give Compound 246 (5.0 mg, yield 27.4%) as a white solid. ¹H NMR (400 MHz, CD₃OD) δ 8.54 (s, 1H), 8.25 (t, J=4.2 Hz, 1H), 7.87 (d, J=8.8 Hz, 2H), 7.53 (d, J=14.2 Hz, 1H), 7.42 (t, J=7.9 Hz, 2H), 7.35 (d, J=6.3 Hz, 4H), 7.29-7.25 (m, 1H), 7.21 (t, J=7.5 Hz, 1H), 7.07 (d, J=7.7 Hz, 2H), 7.02 (d, J=8.6 Hz, 2H), 5.31 (q, J=6.9 Hz, 1H), 4.69 (d, J=6.6 Hz, 1H), 4.22 (d, J=10.5 Hz, 3H), 3.67 (t, J=27.6 Hz, 2H), 2.52-2.35 (m, 2H), 1.64 (dd, J=32.4, 6.9 Hz, 3H). LC/MS (ESI) m/z: 596 (M+H)⁺.

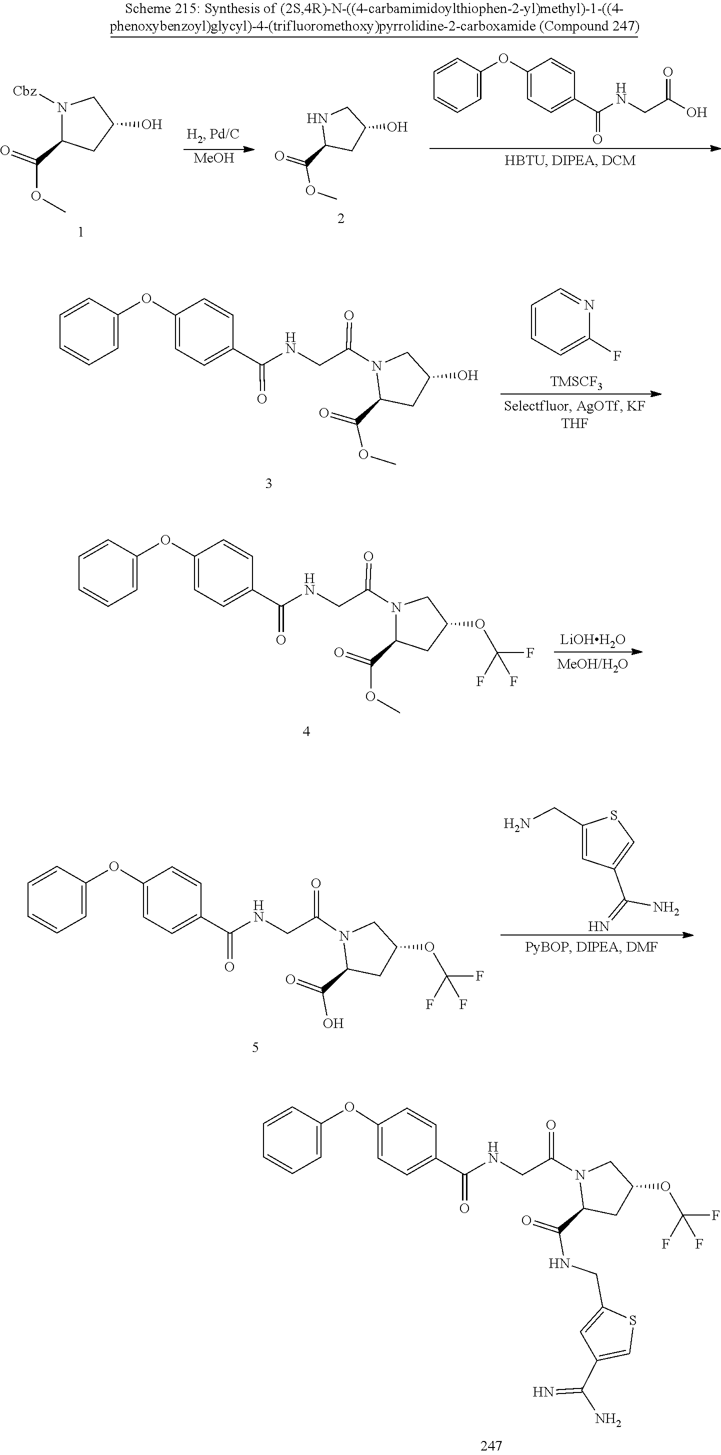

### Step 1: methyl (2S,4R)-4-hydroxypyrrolidine-2-carboxylate

To a solution of 1-benzyl 2-methyl (2S,4R)-4-hydroxypyrrolidine-1,2-dicarboxylate (850 mg, 1.1 mmol) in MeOH (15 mL) was added Pd/C (90 mg, 10% wt.) at room temperature and the mixture was stirred under H₂ atmosphere at room temperature overnight. The mixture was filtered and the filtrate was concentrated to dryness under reduced pressure to give methyl (2S,4R)-4-hydroxypyrrolidine-2-carboxylate (428 mg, yield 97.0%) as a colorless oil, which was used directly in the next step. LC/MS (ESI) (m/z): 146 (M+H)⁺.

### Step 2: methyl (2S,4R)-4-hydroxy-1-((4-phenoxybenzoyl)glycyl)pyrrolidine-2-carboxylate

To a mixture of methyl (2S,4R)-4-hydroxypyrrolidine-2-carboxylate (428 mg, 3.0 mmol) and (4-phenoxybenzoyl)glycine (800 mg, 3.0 mmol) in DMF (12 mL) was added DIPEA (2.2 g, 17 mmol) and HBTU (1.4 g, 3.8 mmol) at 0° C. under N₂ atmosphere and the mixture was stirred at 25° C. for 2 hours. The mixture was diluted with water, extracted with EtOAc twice. The combined organic layers were dried over anhydrous Na₂SO₄, filtered and concentrated to dryness under reduced pressure. The residue was purified by column chromatography on silica gel (PE:EtOAc=2:1) to give methyl (2S,4R)-4-hydroxy-1-((4-phenoxybenzoyl)glycyl)pyrrolidine-2-carboxylate (1.0 g, yield 84.7%) as a colorless oil. LC/MS (ESI) m/z: 399 (M+H)⁺.

### Step 3: methyl (2S,4R)-1-((4-phenoxybenzoyl)glycyl)-4-(trifluoromethoxy)pyrrolidine-2-carboxylate

To a solution of methyl (2S,4R)-4-hydroxy-1-((4-phenoxybenzoyl)glycyl)pyrrolidine-2-carboxylate (1.0 g, 2.5 mmol) in EtOAc (15 mL) was added selectfluor (4.4 g, 12 mmol), AgOTf (6.4 g, 25 mmol), KF (1.5 g, 25 mmol), 2-fluoropyridine (2.4 g, 25 mmol) and TMSCF₃ (3.5 g, 25 mmol) under N₂ atmosphere at 40° C. and the reaction mixture was stirred for 48 hours. The mixture was diluted with water and extracted with EtOAc twice. The combined organic layers were washed with brine, dried over anhydrous Na₂SO₄, filtered and concentrated to dryness under reduced pressure. The residue was purified by column chromatography on silica gel (PE:EtOAc=2:1) to give methyl (2S,4R)-1-((4-phenoxybenzoyl)glycyl)-4-(trifluoromethoxy) pyrrolidine-2-carboxylate (300 mg, yield 25.7%) as a colorless oil. LCMS (ESI) m/z=467 (M+H)⁺.

### Step 4: (2S,4R)-1-((4-phenoxybenzoyl)glycyl)-4-(trifluoromethoxy)pyrrolidine-2-carboxylic acid

To a solution of methyl (2S,4R)-1-((4-phenoxybenzoyl)glycyl)-4-(trifluoromethoxy)pyrrolidine-2-carboxylate (100 mg, 0.21 mmol) in MeOH (2 mL) and water (1 mL) was added LiOH (33 mg, 0.81 mmol) at 0° C. and the mixture was stirred at room temperature for 2 hours. The mixture was acidified with 1N aq.HCl to pH-3, extracted with EtOAc twice. The combined organic layers were washed with brine, dried over anhydrous Na₂SO₄, filtered and concentrated under reduced pressure to dryness to give (2S,4R)-1-((4-phenoxybenzoyl)glycyl)-4-(trifluoromethoxy)pyrrolidine-2-carboxylic acid (90 mg, yield 93.0%) as a colorless oil, which was used directly in the next step. LC/MS (ESI) (m/z): 453 (M+H)⁺.

### Step 5: (2S,4S)-N-((4-carbamimidoylthiophen-2-yl)methyl)-1-((4-phenoxybenzoyl)glycyl)-4-(trifluoromethoxy)pyrrolidine-2-carboxamide (Compound 247)

To a mixture of (2S,4R)-1-((4-phenoxybenzoyl)glycyl)-4-(trifluoromethoxy)pyrrolidine-2-carboxylic acid (50 mg, 0.22 mmol) and 5-(aminomethyl)thiophene-3-carboximidamide (100 mg, 0.66 mmol) in DMF (2 mL) was added DIPEA (170 mg, 1.3 mmol) and PyBOP (148 mg, 0.29 mmol) at 0° C. under N₂ atmosphere and the mixture was stirred at 25° C. for 2 hours. The mixture was diluted with water, extracted with EtOAc twice. The combined organic layers were dried over anhydrous Na₂SO₄, filtered and concentrated to dryness under reduced pressure. The residue was purified by column chromatography on silica gel (DCM:MeOH=8:1) and prep-HPLC to give Compound 247 (10 mg, yield 7.7%) as a colorless oil. ¹H NMR (400 MHz, CD₃OD) δ 8.53 (s, 1H), 8.22 (t, J=9.3 Hz, 1H), 7.84 (d, J=8.7 Hz, 2H), 7.51-7.38 (m, 3H), 7.20 (t, J=7.4 Hz, 1H), 7.06 (d, J=7.8 Hz, 2H), 7.00 (d, J=8.7 Hz, 2H), 5.18 (s, 1H), 4.61-4.55 (m, 3H), 4.27-4.09 (m, 2H), 4.05-3.94 (m, 2H), 2.60 (dd, J=44.9, 34.0 Hz, 1H), 2.44-2.25 (m, 1H). LC/MS (ESI) m/z: 590 (M+H)⁺.

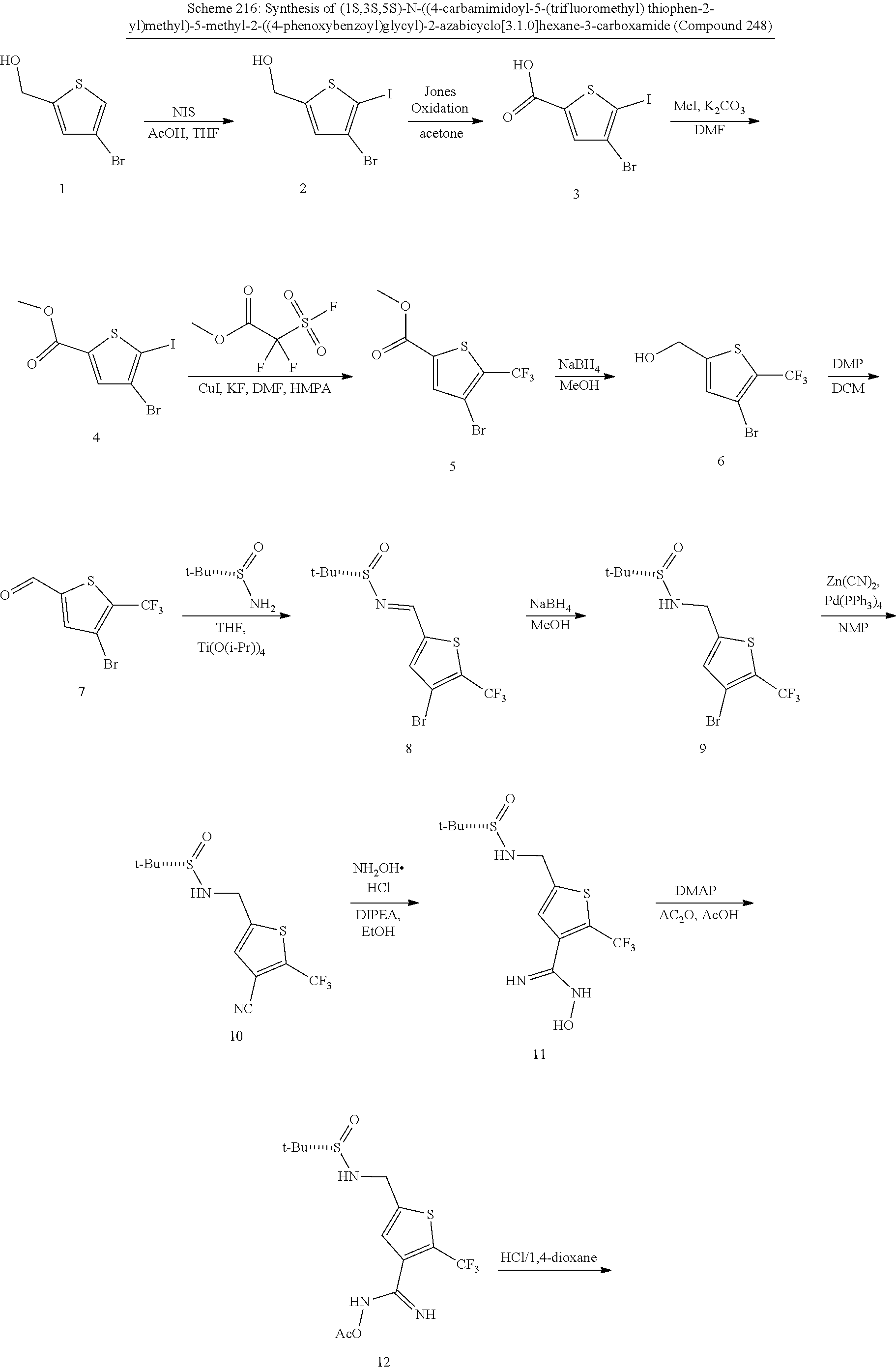

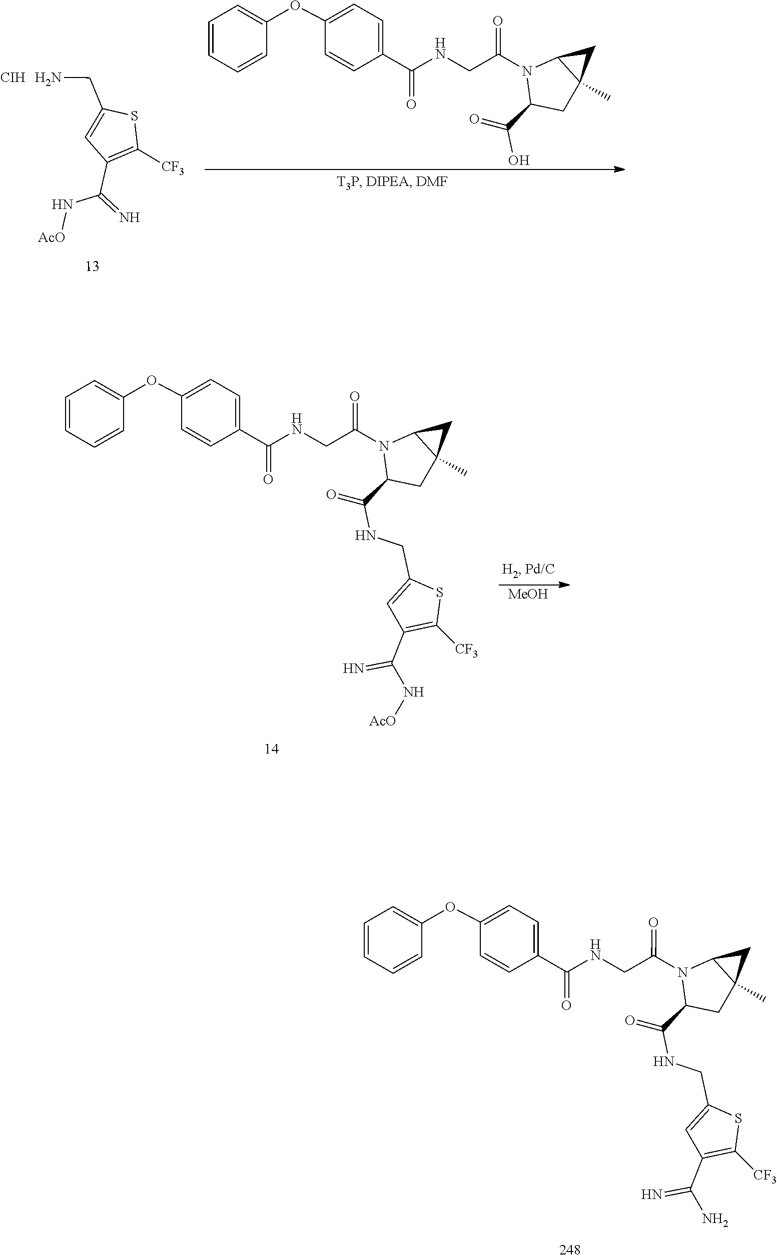 [SMILES: "CIH" ; CC(=O)ONC(=N)c1cc(CN)sc1C(F)(F)F ; C[C@@]12C[C@@H]1N(C(=O)CNC(=O)c1ccc(Oc3ccccc3)cc1)[C@H](C(=O)O)C2 ; "MeOH" ; CC(=O)ONC(=N)c1cc(CNC(=O)[C@@H]2C[C@]3(C)C[C@@H]3N2C(=O)CNC(=O)c2ccc(Oc3ccccc3)cc2)sc1C(F)(F)F ; C[C@@]12C[C@@H]1N(C(=O)CNC(=O)c1ccc(Oc3ccccc3)cc1)[C@H](C(=O)NCc1cc(C(=N)N)c(C(F)(F)F)s1)C2]

### Step 1: (4-bromo-5-iodothiophen-2-yl)methanol (2)

To a mixture of (4-bromothiophen-2-yl)methanol (10 g, 51.8 mmol) in THE (80 mL) and Ac₂O (70 mL) was added NIS (17.5 g, 77.7 mmol) under N₂ atmosphere and the mixture was stirred at room temperature overnight. The mixture was diluted with EtOAc, washed with saturated aq.Na₂S₂O₃ solution and brine, dried over anhydrous Na₂SO₄, filtered and concentrated under reduced pressure to dryness. The residue was purified by flash chromatography on silica gel (PE:EtOAc=10:1) to give (4-bromo-5-iodothiophen-2-yl)methanol (14 g, yield 84.7%) as a yellow solid. LC/MS (ESI) m/z: 301 (M-OH)⁺.

### Step 2: 4-bromo-5-iodothiophene-2-carboxylic acid (3)

To a solution of (4-bromo-5-iodothiophen-2-yl)methanol (11 g, 31.0 mmol) in acetone (100 mL) was added Jones reagent (27 mL, 84.8 mmol) and the reaction mixture was stirred at room temperature for 18 hours. The mixture was diluted with EtOAc, washed with ice-water and brine, dried over anhydrous Na₂SO₄, filtered and concentrated under reduced pressure to give 4-bromo-5-iodothiophene-2-carboxylic acid (11 g, yield 95.8%) as a white solid. LC/MS (ESI) m/z: 232 (M−H)⁺.

### Step 3: methyl 4-bromo-5-iodothiophene-2-carboxylate (4)

To a mixture of 4-bromo-5-iodothiophene-2-carboxylic acid (11 g, 33.0 mmol) and iodomethane (7.0 g, 49.6 mmol) in DMF (100 mL) was added K₂CO₃ (13.7 g, 99.1 mmol) under N₂ atmosphere and the reaction mixture was stirred at room temperature for 2 hours. The mixture was diluted with EtOAc, washed with water and brine, dried over anhydrous Na₂SO₄, filtered and concentrated under reduced pressure to dryness. The residue was purified by flash chromatography on silica gel (PE:EtOAc=10:1) to give methyl 4-bromo-5-iodothiophene-2-carboxylate (7.6 g, yield 66.3%) as a yellow solid.

### Step 4: methyl 4-bromo-5-(trifluoromethyl)thiophene-2-carboxylate (5)

To a mixture of methyl 4-bromo-5-iodothiophene-2-carboxylate (6.0 g, 17.3 mmol) and methyl 2,2-difluoro-2-(fluorosulfonyl)acetate (13.3 g, 69.2 mmol) in DMF (27 mL) and HMPA (27 mL) was added copper iodide (3.3 g, 17.3 mmol) and KF (3 g, 51.7 mmol) at 0° C. and the mixture was stirred at 100° C. for 16 hours. The mixture was diluted with water and extracted with EtOAc twice. The combined organic layers were washed with brine, dried over anhydrous Na₂SO₄, filtered and concentrated under reduced pressure to dryness. The residue was purified by chromatography on silica gel (PE:EtOAc=10:1) to give methyl 4-bromo-5-(trifluoromethyl)thiophene-2-carboxylate (3.4 g, yield 68.0%) as a colorless oil.

### Step 5: (4-bromo-5-(trifluoromethyl)thiophen-2-yl)methanol (6)

To a mixture of methyl 4-bromo-5-(trifluoromethyl)thiophene-2-carboxylate (3.4 g, 11.762 mmol) in MeOH (34 mL) was added NaBH₄ (1.9 g, 47.1 mmol), and the mixture was stirred at 25° C. for 3 hours.

The mixture was diluted with water and extracted with DCM twice. The combined organic layers were washed with brine, dried over anhydrous Na₂SO₄, filtered and concentrated under reduced pressure to dryness. The residue was purified by flash chromatography on silica gel (PE:EtOAc=10:1) to afford (4-bromo-5-(trifluoromethyl) thiophen-2-yl)methanol (2.9 g, yield 94.4%) as a colorless oil.

### Step 6: 4-bromo-5-(trifluoromethyl)thiophene-2-carbaldehyde (7)

To a solution of (4-bromo-5-(trifluoromethyl)thiophen-2-yl)methanol (2.5 g, 9.6 mmol) in DCM (25 mL) was added Dess-Martin periodinane (8.1 g, 19.2 mmol) at 0° C., and the mixture was stirred 60° C. for 16 hours. The mixture was diluted with water and extracted with EtOAc twice. The combined organic layers were washed with brine, dried over anhydrous Na₂SO₄, filtered and concentrated under reduced pressure to dryness. The residue was purified by flash chromatography on silica gel (PE:EtOAc=1:1) to give 4-bromo-5-(trifluoromethyl) thiophene-2-carbaldehyde (1.7 g, yield 68.5%) as a colorless oil.

### Step 7: (S,E)-N-((4-bromo-5-(trifluoromethyl)thiophen-2-yl)methylene)-2-methylpropane-2-sulfinamide (8)

To a mixture of 4-bromo-5-(trifluoromethyl)thiophene-2-carbaldehyde (1.7 g, 6.6 mmol) and (S)-2-methylpropane-2-sulfinamide (2.4 g, 19.7 mmol) in THE (20 mL) was added Ti(OiPr)₄ (11.2 g, 39.4 mmol) under N₂ atmosphere and the reaction mixture was stirred at 70° C. for 2 hours. The mixture was diluted with EtOAc, washed with water and brine, dried over anhydrous Na₂SO₄, filtered and concentrated under reduced pressure to give (S,E)-N-((4-bromo-5-(trifluoromethyl)thiophen-2-yl)methylene)-2-methylpropane-2-sulfinamide (2.0 g, yield 84.1%) as a white solid. LC/MS (ESI) m/z: 363 (M+H)⁺.

### Step 8: (S)-N-((4-bromo-5-(trifluoromethyl)thiophen-2-yl)methyl)-2-methylpropane-2-sulfinamide (9)

To a mixture of (S,E)-N-((4-bromo-5-(trifluoromethyl)thiophen-2-yl)methylene)-2-methylpropane-2-sulfinamide (2.0 g, 5.5 mmol) in MeOH (20 mL) was added NaBH₄ (627 mg, 16.6 mmol), and the mixture was stirred at 25° C. for 3 hours. The mixture was diluted with water and extracted with DCM twice. The combined organic layers were washed with brine, dried over anhydrous Na₂SO₄, filtered and concentrated under reduced pressure to dryness. The residue was purified by flash chromatography on silica gel (PE:EtOAc=10:1) to afford (S)-N-((4-bromo-5-(trifluoromethyl)thiophen-2-yl)methyl)-2-methylpropane-2-sulfinamide (1.4 g, Yield 69.6%) as a colorless oil. LC/MS (ESI) m/z: 365 (M+H)⁺.

### Step 9: (S)-N-((4-cyano-5-(trifluoromethyl)thiophen-2-yl)methyl)-2-methylpropane-2-sulfinamide (10)

To a solution of (S)-N-((4-bromo-5-(trifluoromethyl)thiophen-2-yl)methyl)-2-methylpropane-2-sulfinamide (1.4 g, 3.8 mmol) in NMP (3 mL) was added Zn(CN)₂ (903 mg, 7.7 mmol) and Pd(PPh₃)₄ (666 mg, 0.57 mmol). The reaction mixture was heated to 110° C. under N₂ atmosphere and the reaction mixture was stirred at 110° C. for 16 hours. The mixture was diluted with EtOAc and washed with water. The aqueous layer was extracted with EtOAc and the combined organic layers were washed with brine, dried over anhydrous Na₂SO₄, filtered and concentrated to dryness under reduced pressure. The residue was purified by flash chromatography on silica gel (PE:EtOAc=1:1) to afford (S)-N-((4-cyano-5-(trifluoromethyl)thiophen-2-yl)methyl)-2-methylpropane-2-sulfinamide (1.1 g, yield 92.4%) as a colorless oil. LC/MS (ESI) m/z: 311 (M+H)⁺.

### Step 10: (S)-5-(((tert-butylsulfinyl)amino)methyl)-N-hydroxy-2-(trifluoromethyl)thiophene-3-carboximidamide (11)

To a mixture of (S)-N-((4-cyano-5-(trifluoromethyl)thiophen-2-yl)methyl)-2-methylpropane-2-sulfinamide (600 mg, 1.9 mmol) in EtOH (6 mL) was added DIPEA (1.0 mL, 5.8 mmol) and NH₂OH·HCl (336 mg, 4.8 mmol) and the mixture was stirred at 25° C. for 16 hours. The mixture was diluted with EtOAc, washed with brine, dried over anhydrous Na₂SO₄, filtered and concentrated to dryness under reduced pressure. The residue was purified by flash chromatography on silica gel (PE:EtOAc=1:1) to give (S)-5-(((tert-butylsulfinyl)amino)methyl)-N-hydroxy-2-(trifluoromethyl) thiophene-3-carboximidamide (590 mg, yield 88.9%) as a white solid. LC/MS (ESI) m/z: 344 (M+H)⁺.

### Step 11: (S)-N-acetoxy-5-(((tert-butylsulfinyl)amino)methyl)-2-(trifluoromethyl)thiophene-3-carboximidamide (12)

To a mixture of (S)-5-(((tert-butylsulfinyl)amino)methyl)-N-hydroxy-2-(trifluoromethyl) thiophene-3-carboximidamide (590 mg, 1.7 mmol) in AcOH (6 mL) was added Ac₂O (6 mL) and DMAP (20 mg) and the mixture was stirred at 25° C. for 16 hours. The mixture was diluted with EtOAc, washed with water and brine, dried over anhydrous Na₂SO₄, filtered and concentrated to dryness under reduced pressure. The residue was purified by flash chromatography on silica gel (DCM:MeOH=20:1) to afford (S)-N-acetoxy-5-(((tert-butylsulfinyl)amino)methyl)-2-(trifluoromethyl)thiophene-3-carboximidamide (557 mg, yield 84.1%) as a colorless oil. LC/MS (ESI) m/z: 386 (M+H)⁺.

### Step 12: N-acetoxy-5-(aminomethyl)-2-(trifluoromethyl)thiophene-3-carboximidamide hydrochloride (13)

A mixture of (S)-N-acetoxy-5-(((tert-butylsulfinyl)amino)methyl)-2-(trifluoromethyl) thiophene-3-carboximidamide (120 mg, 0.30 mmol) in HCl/1,4-dioxane (3 mL) was stirred at room temperature for 2 hours. The reaction mixture was concentrated to dryness under reduced pressure, co-evaporated with DCM and dried under vacuum to give N-acetoxy-5-(aminomethyl)-2-(trifluoromethyl)thiophene-3-carboximidamide hydrochloride (99.0 mg, yield 100%) as a white solid, which was used directly in the next reaction without further purification. LC/MS (ESI) m/z: 282 (M+H)⁺.

### Step 13: (1S,3S,5S)-N-((4-(N-acetoxycarbamimidoyl)-5-(trifluoromethyl)thiophen-2-yl) methyl)-5-methyl-2-((4-phenoxybenzoyl)glycyl)-2-azabicyclo[3.1.0]hexane-3-carboxamide (14)

To a mixture of N-acetoxy-5-(aminomethyl)-2-(trifluoromethyl)thiophene-3-carboximidamide hydrochloride (40 mg, 0.14 mmol) and (1S,3S,5S)-5-methyl-2-((4-phenoxybenzoyl)glycyl)-2-azabicyclo[3.1.0]hexane-3-carboxylic acid (50 mg, 0.13 mmol) in DMF (3 mL) was added DIPEA (0.12 mL, 0.71 mmol) and T₃P (136 mg, 0.21 mmol, 50% wt. in EtOAc) at 0° C. under N₂ atmosphere and the mixture was stirred at 25° C. for 3 hours. The mixture was quenched with water and extracted with EtOAc twice. The combined organic layers were washed with brine, dried over anhydrous Na₂SO₄, filtered and concentrated to dryness under reduced pressure. The residue was purified by prep-TLC (DCM:MeOH=15:1) to give (1S,3S,5S)-N-((4-(N-acetoxycarbamimidoyl)-5-(trifluoromethyl)thiophen-2-yl)methyl)-5-methyl-2-((4-phenoxybenzoyl)glycyl)-2-azabicyclo[3.1.0]hexane-3-carboxamide (28 mg, yield 33.3%) as a colorless oil. LC/MS (ESI) m/z: 658 (M+H)⁺.

### Step 14: (1S,3S,5S)-N-((4-carbamimidoyl-5-(trifluoromethyl)thiophen-2-yl)methyl)-5-methyl-2-((4-phenoxybenzoyl)glycyl)-2-azabicyclo[3.1.0]hexane-3-carboxamide (Compound 248)

To a mixture of (1 S,3S,5S)-N-((4-(N-acetoxycarbamimidoyl)-5-(trifluoromethyl)thiophen-2-yl) methyl)-5-methyl-2-((4-phenoxybenzoyl)glycyl)-2-azabicyclo[3.1.0]hexane-3-carboxamide (14 mg, 0.02 mmol) in MeOH (4 mL) was added Pd/C (5 mg, 10% wt.), and the mixture was degassed under N₂ atmosphere for three times and stirred under a H₂ balloon at 25° C. for 0.5 hours. The mixture was filtered, and the filtrate was concentrated under reduced pressure to dryness. The residue was purified by prep-HPLC to give Compound 248 (0.8 mg, yield 6.3%) as a white solid. ¹H NMR (400 MHz, CD₃OD) δ 8.52 (s, 1H), 7.87 (d, J=8.8 Hz, 2H), 7.42 (t, J=7.9 Hz, 2H), 7.22 (dd, J=16.7, 9.2 Hz, 2H), 7.07 (d, J=7.8 Hz, 2H), 7.02 (d, J=8.7 Hz, 2H), 4.81 (d, J=4.1 Hz, 1H), 4.57 (s, 2H), 4.33 (s, 2H), 3.42 (dd, J=6.0, 2.5 Hz, 1H), 2.42 (t, J=12.8 Hz, 1H), 2.16 (dd, J=13.8, 2.5 Hz, 1H), 1.30 (s, 3H), 1.14-1.10 (m, 1H), 0.82 (t, J=6.4 Hz, 1H). LC/MS (ESI) m/z: 600 (M+H)⁺.

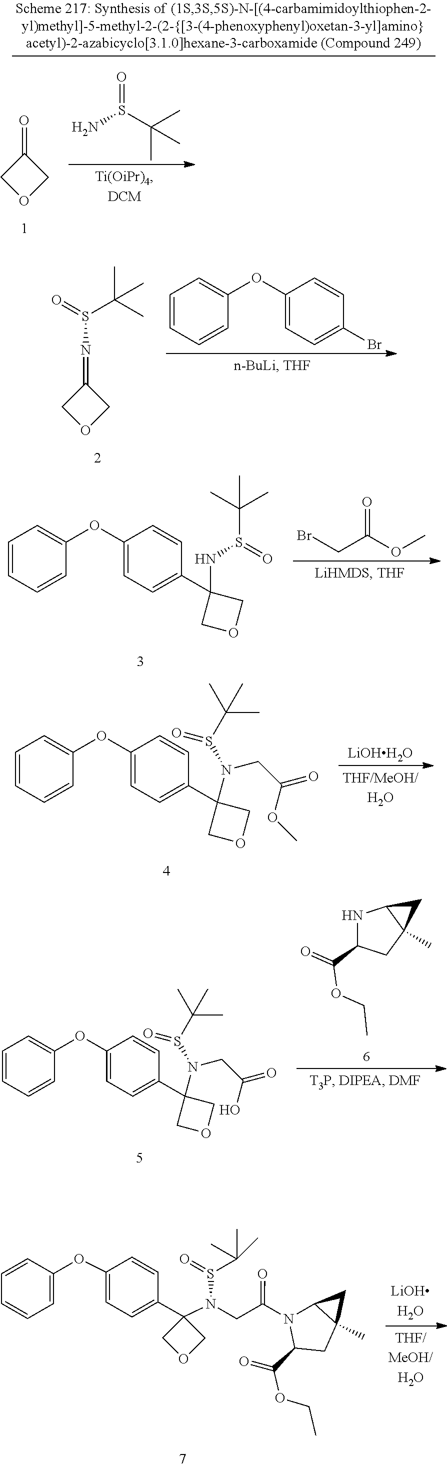

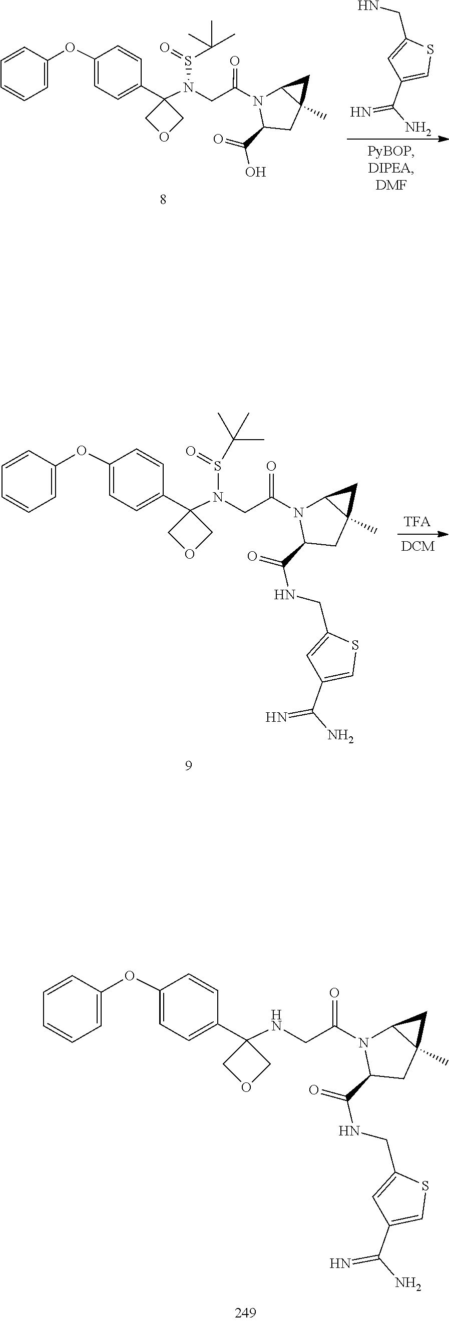

### Step 1: (R)-2-methyl-N-(oxetan-3-ylidene)propane-2-sulfinamide

To a solution of 3-oxetanone (6.2 g, 86.0 mmol) in DCM (120 mL) was added (R)-2-methylpropane-2-sulfinamide (20.9 g, 172.1 mmol) and titanium tetraisopropanolate (49.1 g, 172.8 mmol) at 0° C. and the reaction mixture was stirred at 45° C. for 16 hours. The mixture was quenched with ice-water and extracted with DCM twice. The combined organic layers were washed with brine, dried over anhydrous Na₂SO₄, filtered and concentrated to dryness. The residue was purified by chromatography on silica gel (PE:EtOAc=15:1) to give (R)-2-methyl-N-(oxetan-3-ylidene)propane-2-sulfinamide (6.2 g, yield 41.1%) as a yellow oil. LC/MS (ESI) m/z: 176(M+H)⁺.

### Step 2: (R)-2-methyl-N-[3-(4-phenoxyphenyl)oxetan-3-yl]propane-2-sulfinamide

To a solution of 4-bromophenyl phenyl ether (4.26 g, 17.1 mmol) in THE (90 mL) was added n-BuLi (6.39 mL, 15.98 mmol, 2.5 M) at −78° C. and the mixture was stirred under N₂ atmosphere for 1 hour before a solution of (R)-2-methyl-N-(oxetan-3-ylidene) propane-2-sulfinamide (2.0 g, 11.41 mmol) in THE (4 mL) was added and the resulting mixture was stirred at −78° C. for 0.5 hours under N₂ atmosphere. The mixture was quenched with saturated aq.NH₄Cl solution and extracted with EtOAc twice. The combined organic layers were washed with brine, dried over anhydrous Na₂SO₄, filtered and concentrated to dryness under reduced pressure. The residue was purified by column chromatography on silica gel (DCM:MeOH=40:1) to give (R)-2-methyl-N-[3-(4-phenoxyphenyl)oxetan-3-yl]propane-2-sulfinamide (3.7 g, yield 93.9%) as a yellow oil. LC/MS (ESI) m/z: 346(M+H)⁺.

### Step 3: methyl 2-{[(R)-2-methylpropane-2-sulfinyl][3-(4-phenoxyphenyl)oxetan-3-yl]amino}acetate

To a solution of (R)-2-methyl-N-[3-(4-phenoxyphenyl)oxetan-3-yl]propane-2-sulfinamide (1.5 g, 4.34 mmol) in DMF (15 mL) was added lithium bis(trimethylsilyl)amide (7.60 mL, 7.60 mmol, 1 M at −20° C. and the mixture was stirred under N₂ atmosphere for 0.5 hours. Then methyl 2-bromoacetate (1.33 g, 8.68 mmol) was added and the mixture was stirred at −20° C. for 1 hour under N₂ atmosphere. The mixture was quenched with ice-water and extracted with EtOAc twice. The combined organic layers were washed with brine, dried over anhydrous Na₂SO₄, filtered and concentrated to dryness under reduced pressure. The residue was purified by column chromatography on Al₂O₃ (PE:EtOAc=2:1) to give methyl 2-{[(R)-2-methylpropane-2-sulfinyl][3-(4-phenoxyphenyl)oxetan-3-yl]amino}acetate (680 mg, yield 37.5%) as a yellow oil. LC/MS (ESI) m/z: 418(M+H)⁺.

### Step 4: {[(R)-2-methylpropane-2-sulfinyl][3-(4-phenoxyphenyl)oxetan-3-yl]amino}acetic acid

To a solution of methyl 2-{[(R)-2-methylpropane-2-sulfinyl][3-(4-phenoxyphenyl)oxetan-3-yl]amino}acetate (380 mg, 0.91 mmol) in MeOH (3 mL)/THF (1.5 mL) was added a solution of LiOH·H₂O (115 mg, 2.73 mmol) in H₂O (1.5 mL) and the mixture was stirred at room temperature for 2 hours. The mixture was concentrated to dryness under reduced pressure to give {[(R)-2-methylpropane-2-sulfinyl][3-(4-phenoxyphenyl)oxetan-3-yl]amino}acetic acid (360 mg, yield 98.0%) as a yellow oil, which was used directly in the next step without further purification. LC/MS (ESI) m/z: 404(M+H)⁺.

### Step 5: {[(R)-2-methylpropane-2-sulfinyl][3-(4-phenoxyphenyl)oxetan-3-yl]amino}acetic

To a mixture of {[(R)-2-methylpropane-2-sulfinyl][3-(4-phenoxyphenyl)oxetan-3-yl]amino}acetic acid (360 mg, 0.89 mmol) and (1 S,3S,5S)-5-methyl-2-azabicyclo[3.1.0]hexane-3-carboxylate (181 mg, 1.07 mmol) in DMF (6 mL) was added DIPEA (692 mg, 5.35 mmol) and T₃P (1.14 g, 1.78 mmol, 50% wt. in EtOAc) under N₂ atmosphere and the mixture was stirred at room temperature for 1 hour. The mixture was diluted with EtOAc, washed with saturated aq.NaHCO₃ solution and brine, dried over anhydrous Na₂SO₄, filtered and concentrated under reduced pressure to dryness. The residue was purified by prep-TLC (PE:EtOAc=1:1) to give ethyl (1S,3S,5S)-5-methyl-2-{2-[(2-methylpropane-2-sulfinyl)[3-(4-phenoxyphenyl)oxetan-3-yl]amino]acetyl}-2-azabicyclo[3.1.0]hexane-3-carboxylate (45 mg, yield 9.1%) as a light oil. LC/MS (ESI) m/z: 555 (M+H)⁺.

### Step 6: (1S,3S,5S)-5-methyl-2-{2-[(2-methylpropane-2-sulfinyl)[3-(4-phenoxyphenyl)oxetan-3-yl]amino]acetyl}-2-azabicyclo[3.1.0]hexane-3-carboxylic acid

To a solution of ethyl (1 S,3S,5S)-5-methyl-2-{2-[(2-methylpropane-2-sulfinyl)[3-(4-phenoxyphenyl)oxetan-3-yl]amino]acetyl}-2-azabicyclo[3.1.0]hexane-3-carboxylate (45 mg, 0.081 mmol) in MeOH (2 mL)/THF (1 mL) was added a solution of LiOH·H₂O (14 mg, 0.32 mmol) in H₂O (1 mL) and the mixture was stirred at 30° C. for 4 hours. The mixture was concentrated to dryness under reduced pressure to give (1 S,3S,5S)-5-methyl-2-{2-[(2-methylpropane-2-sulfinyl)[3-(4-phenoxyphenyl)oxetan-3-yl]amino]acetyl}-2-azabicyclo[3.1.0]hexane-3-carboxylic acid (40 mg, yield 93.6%) as a yellow oil, which was used directly in the next step without further purification. LC/MS (ESI) m/z: 527(M+H)⁺.

### Step 7: (1S,3S,5S)-N-[(4-carbamimidoylthiophen-2-yl)methyl]-5-methyl-2-{2-[(2-methylpropane-2-sulfinyl)[3-(4-phenoxyphenyl)oxetan-3-yl]amino]acetyl}-2-azabicyclo[3.1.0]hexane-3-carboxamide

To a mixture of (1 S,3S,5S)-5-methyl-2-{2-[(2-methylpropane-2-sulfinyl)[3-(4-phenoxyphenyl) oxetan-3-yl]amino]acetyl}-2-azabicyclo[3.1.0]hexane-3-carboxylic acid (34 mg, 0.065 mmol) and 5-(aminomethyl)thiophene-3-carboximidamide (30 mg, 0.19 mmol) in DMF (3 mL) was added DIPEA (0.056 mL, 0.32 mmol) and PyBOP (50 mg, 0.097 mmol) at 0° C. under N₂ atmosphere and the mixture was stirred at room temperature for 0.5 hours. The mixture was quenched with water and extracted twice with EtOAc. The combined organic layers were washed with brine, dried over anhydrous Na₂SO₄, filtered and concentrated to dryness under reduced pressure. The residue was purified by prep-TLC (DCM:MeOH=20:1) to give (1 S,3S,5S)-N-[(4-carbamimidoylthiophen-2-yl)methyl]-5-methyl-2-{2-[(2-methylpropane-2-sulfinyl)[3-(4-phenoxyphenyl)oxetan-3-yl]amino]acetyl}-2-azabicyclo[3.1.0]hexane-3-carboxamide (13 mg, yield 30.3%) as a light oil. LC/MS (ESI) m/z: 664 (M+H)⁺.

### Step 8: (1S,3S,5S)-N-[(4-carbamimidoylthiophen-2-yl)methyl]-5-methyl-2-(2-{[3-(4-phenoxyphenyl)oxetan-3-yl]amino}acetyl)-2-azabicyclo[3.1.0]hexane-3-carboxamide (Compound 249)

To a solution of (1S,3S,5S)-N-[(4-carbamimidoylthiophen-2-yl)methyl]-5-methyl-2-{2-[(2-methylpropane-2-sulfinyl)[3-(4-phenoxyphenyl)oxetan-3-yl]amino]acetyl}-2-azabicyclo[3.1.0]hexane-3-carboxamide (12 mg, 0.018 mmol) in DCM (1.2 mL) was added TFA (0.4 mL) and stirred at 0° C. for 0.5 hours. The mixture was concentrated to dryness under reduced pressure. The residue was purified by prep-HPLC to give Compound 249 (1.3 mg, yield 12.8%) as a white solid. ¹H NMR (400 MHz, CD₃OD) δ 8.23 (s, 1H), 7.47-7.34 (m, 5H), 7.13 (t, J=7.4 Hz, 1H), 7.05-6.99 (m, 4H), 4.94-4.92 (m, 2H), 4.72-4.70 (m, 1H), 4.58 (d, J=15.7 Hz, 2H), 4.47 (d, J=15.2 Hz, 2H), 3.46-3.37 (m, 2H), 3.10-3.06 (m, 1H), 2.40-2.34 (m, 1H), 2.10-2.05 (m, 1H), 1.23 (s, 3H), 1.19 (dd, J=5.6, 5.6 Hz, 1H), 0.68 (t, J=5.4 Hz, 1H). LC/MS (ESI) m/z:560 (M+H)⁺.

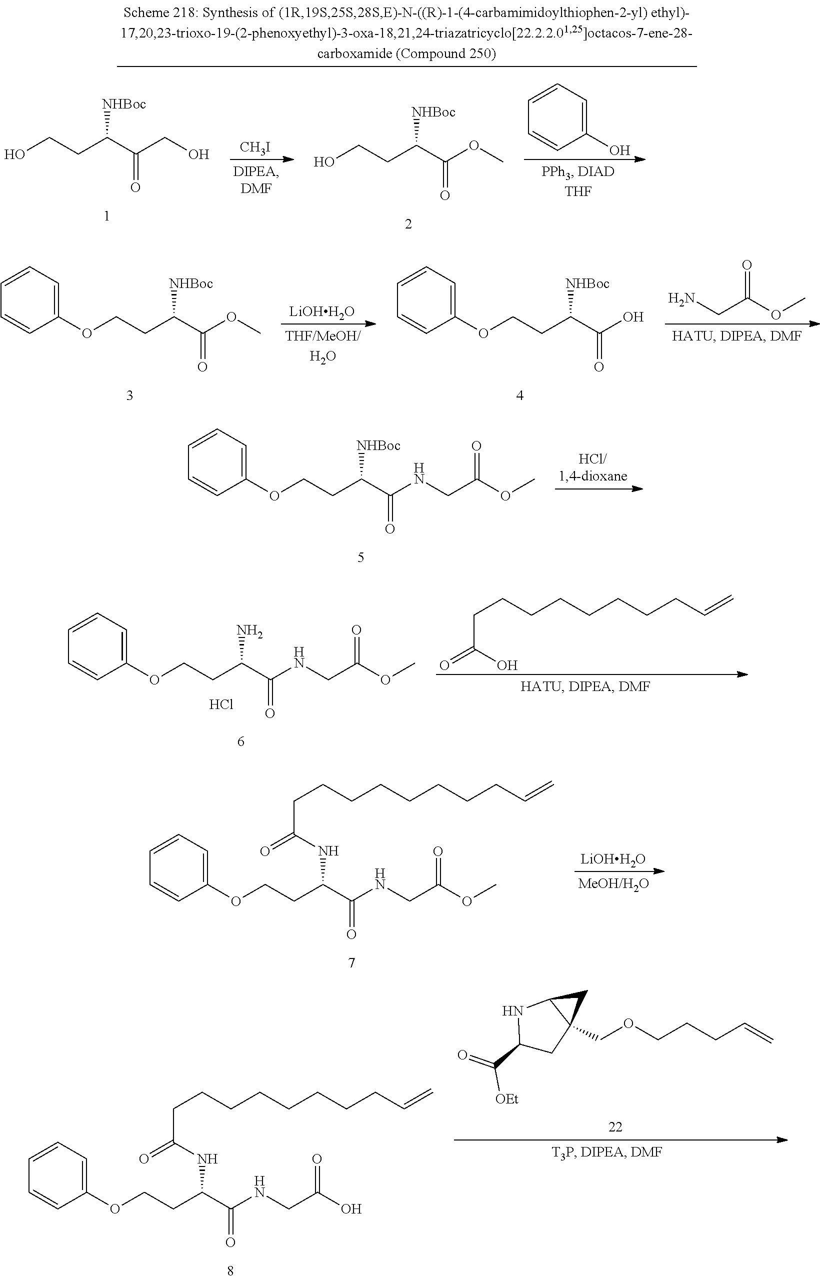

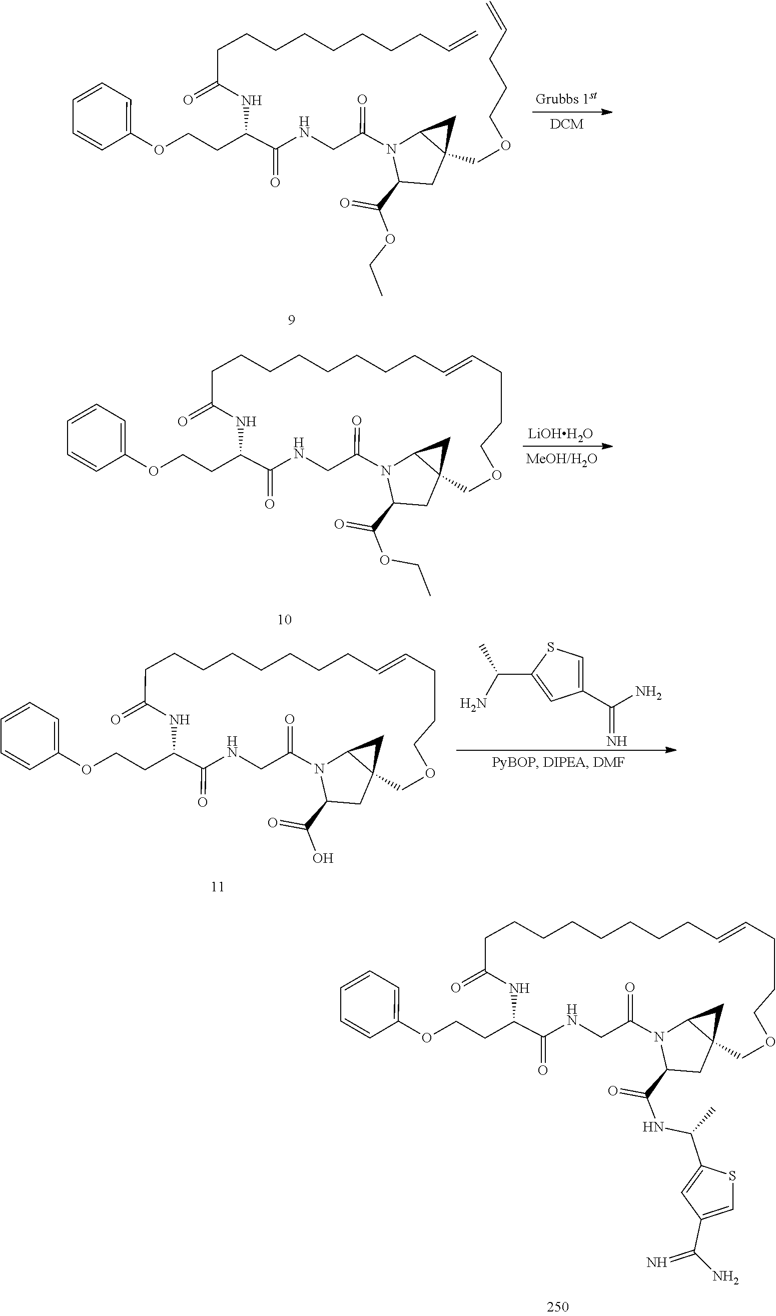

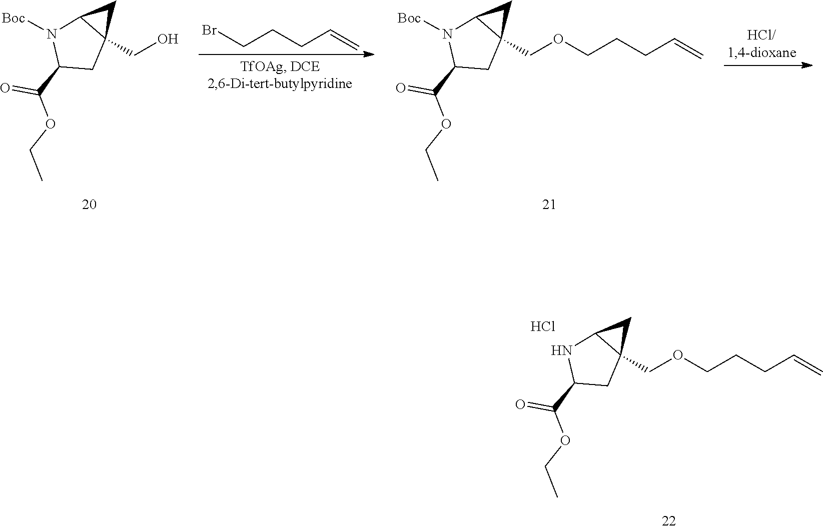

### Step 1: methyl (tert-butoxycarbonyl)-L-homoserinate (2)

To a mixture of (tert-butoxycarbonyl)-L-homoserine (10.0 g, 45.66 mmol) and CH₃I (4.3 mL, 68.49 mmol) in DMF (100 mL) was added DIPEA (23 mL, 136.98 mmol) and the mixture was stirred at 25° C. for 16 hours. The mixture was diluted with EtOAc and washed with aq.NH₄Cl twice. The combined organic layers were dried over anhydrous Na₂SO₄, filtered and concentrated under reduced pressure to dryness. The residue was purified by column chromatography on silica gel (PE:EtOAc=1:1) to give methyl (tert-butoxycarbonyl)-L-homoserinate (4.0 g, yield 37.7%) as a colorless oil. LC/MS (ESI) m/z: 234 (M+H)⁺.

### Step 2: methyl N-(tert-butoxycarbonyl)-O-phenyl-L-homoserinate (3)

To a mixture of methyl (tert-butoxycarbonyl)-L-homoserinate (4.0 g, 17.17 mmol) and phenol (3.23 g, 34.34 mmol) in THE (50.0 mL) was added PPh₃ (13.5 g, 51.51 mmol) and DIAD (10.4 g, 51.51 mmol) at 0° C. under N₂ atmosphere and the mixture was stirred at 30° C. for 16 hours. The mixture was diluted with water and extracted with EtOAc twice. The combined organic layers were dried over anhydrous Na₂SO₄, filtered and concentrated to dryness under reduced pressure. The residue was purified by column chromatography on silica gel (PE:EtOAc=4:1) to give methyl N-(tert-butoxycarbonyl)-O-phenyl-L-homoserinate (4.37 g, yield 82.4%) as a colorless oil. LC/MS (ESI) m/z: 310 (M+H)⁺.

### Step 3: N-(tert-butoxycarbonyl)-O-phenyl-L-homoserine (4)

To a solution of methyl N-(tert-butoxycarbonyl)-O-phenyl-L-homoserinate (1.37 g, 4.43 mmol) in MeOH (12 mL) and water (4 mL) was added lithium hydroxide (280 mg, 6.65 mmol) at 0° C. and the mixture was stirred at room temperature for 2 hours. The mixture was concentrated to dryness and the residue was diluted with water, washed with MTBE twice. The residue was acidified with 1N aq.HCl to pH-3, extracted with EtOAc twice. The combined organic layers were washed with brine, dried over anhydrous Na₂SO₄, filtered and concentrated under reduced pressure to dryness to give N-(tert-butoxycarbonyl)-O-phenyl-L-homoserine (1.2 g, yield 92.3%) as a yellow oil, which was used directly in the next step without further purification. LC/MS (ESI) (m/z): 296 (M+H)⁺.

### Step 4: methyl N-(tert-butoxycarbonyl)-O-phenyl-L-homoserylglycinate (5)

To a mixture of N-(tert-butoxycarbonyl)-O-phenyl-L-homoserine (1.2 g, 4.07 mmol) and methyl glycinate (770 mg, 6.10 mmol) in DMF (15 mL) was added HATU (2.3 g, 6.10 mmol) and DIPEA (3.35 mL, 20.35 mmol) under N₂ atmosphere and the reaction mixture was stirred at room temperature overnight. The mixture was diluted with EtOAc, washed with water and brine, dried over anhydrous Na₂SO₄, filtered and concentrated under reduced pressure to dryness. The residue was purified by flash chromatography on silica gel (PE:EtOAc=1:1) to give methyl N-(tert-butoxycarbonyl)-O-phenyl-L-homoserylglycinate (1.0 g, yield 67.2%) as a colorless oil. LC/MS (ESI) m/z: 367 (M+H)⁺.

### Step 5: methyl O-phenyl-L-homoserylglycinate hydrochloride (6)

A solution of methyl N-(tert-butoxycarbonyl)-O-phenyl-L-homoserylglycinate (1.0 g, 2.73 mmol) in HCl/1,4-dioxane (10 mL) was stirred at room temperature for 1 hour. The mixture was concentrated to dryness under reduced pressure to give methyl O-phenyl-L-homoserylglycinate hydrochloride (700 mg, yield 84.5%) as a colorless oil, which was directly used in the next reaction without further purification. LC/MS (ESI) m/z: 267 (M+H)⁺.

### Step 6: methyl O-phenyl-N-(undec-10-enoyl)-L-homoserylglycinate (7)

To a mixture of methyl O-phenyl-L-homoserylglycinate hydrochloride (756 mg, 2.84 mmol) and undec-10-enoic acid (784 mg, 4.26 mmol) in DMF (10 mL) was added HATU (1.62 g, 4.26 mmol) and DIPEA (2.34 mL, 14.20 mmol) under N₂ atmosphere and the reaction mixture was stirred at room temperature overnight. The mixture was diluted with EtOAc, washed with water and brine, dried over anhydrous Na₂SO₄, filtered and concentrated under reduced pressure to dryness. The residue was purified by flash chromatography on silica gel (PE:EtOAc=1:1) to give methyl O-phenyl-N-(undec-10-enoyl)-L-homoserylglycinate (1.05 g, yield 85.4%) as a white solid. LC/MS (ESI) m/z: 433 (M+H)⁺.

### Step 7: O-phenyl-N-(undec-10-enoyl)-L-homoserylglycine (8)

To a solution of methyl O-phenyl-N-(undec-10-enoyl)-L-homoserylglycinate (1.05 g, 2.43 mmol) in MeOH (9 mL) and water (3 mL) was added lithium hydroxide (308 mg, 7.29 mmol) at 0° C. and the mixture was stirred at room temperature for 2 hours. The mixture was concentrated to dryness and the residue was diluted with water, washed with MTBE twice. The residue was acidified with 1N aq.HCl to pH-3, extracted with EtOAc twice. The combined organic layers were washed with brine, dried over anhydrous Na₂SO₄, filtered and concentrated to dryness under reduced pressure to give 0-phenyl-N-(undec-10-enoyl)-L-homoserylglycine (950 mg, yield 98.1%) as a colorless oil, which was used directly in the next step. LC/MS (ESI) (m/z): 419 (M+H)⁺.

### Step 8: (S)-N-((R)-1-(4-(N-acetoxycarbamimidoyl)thiophen-2-yl)ethyl)-7-((4′-methyl-[1,1′-biphenyl]-3-carbonyl)glycyl)-1,4-dioxa-7-azaspiro[4.4]nonane-8-carboxamide (9)

To a mixture of O-phenyl-N-(undec-10-enoyl)-L-homoserylglycine (968 mg, 2.31 mmol) and ethyl (1S,3S,5R)-5-((pent-4-en-1-yloxy)methyl)-2-azabicyclo[3.1.0]hexane-3-carboxylate HCl salt (879 mg, 3.47 mmol) in DMF (20 mL) was added DIPEA (1.90 mL, 11.55 mmol) and T₃P (4.4 g, 6.93 mmol, 50% wt. in EtOAc) under N₂ atmosphere and the reaction mixture was stirred at room temperature for 2 hours. The mixture was diluted with EtOAc, washed with saturated aq.NaHCO₃ and brine, dried over anhydrous Na₂SO₄, filtered and concentrated under reduced pressure to dryness. The residue was purified by column chromatography on silica gel (DCM:MeOH=97:3) to give ethyl (1S,3S,5R)-5-((pent-4-en-1-yloxy)methyl)-2-(O-phenyl-N-(undec-10-enoyl)-L-homoserylglycyl)-2-azabicyclo[3.1.0]hexane-3-carboxylate (1.32 g, yield 87.4%) as a brown oil. LC/MS (ESI) m/z: 654 (M+H)⁺.

### Step 9: ethyl (1R,19S,25S,28S,E)-17,20,23-trioxo-19-(2-phenoxyethyl)-3-oxa-18,21,24-triazatricyclo[22.2.2.0^{1,25}]octacos-7-ene-28-carboxylate (10)

To a solution of ethyl (1 S,3S,5R)-5-((pent-4-en-1-yloxy)methyl)-2-(O-phenyl-N-(undec-10-enoyl)-L-homoserylglycyl)-2-azabicyclo[3.1.0]hexane-3-carboxylate (700 mg, 1.07 mmol) in dry DCM (70 mL) was added Grubbs 1^{st} catalyst (220 mg, 0.27 mmol) and the mixture was stirred at 28° C. for 16 hours under N₂ atmosphere. The mixture was concentrated to dryness under reduced pressure and the residue was purified by flash chromatography (DCM:MeOH=98:2) to give ethyl (1R,19S,25S,28S,E)-17,20,23-trioxo-19-(2-phenoxyethyl)-3-oxa-18,21,24-triazatricyclo[22.2.2.0^{1,25}]octacos-7-ene-28-carboxylate (260 mg, yield 38.8%) as a black oil. LC/MS (ESI) m/z: 626 (M+H)⁺.

### Step 10: (1R,19S,25S,28S,E)-17,20,23-trioxo-19-(2-phenoxyethyl)-3-oxa-18,21,24-triazatricyclo[22.2.2.0^{1,25}]octacos-7-ene-28-carboxylic acid (11)

To a solution of ethyl (1R,19S,25S,28S,E)-17,20,23-trioxo-19-(2-phenoxyethyl)-3-oxa-18,21,24-triazatricyclo[22.2.2.0^{1,25}]octacos-7-ene-28-carboxylate (490 mg, 0.78 mmol) in MeOH (3 mL) and water (1 mL) was added lithium hydroxide (50 mg, 1.18 mmol) at 0° C. and the mixture was stirred at room temperature for 16 hours. The mixture was concentrated to dryness and the residue was diluted with water, washed with MTBE twice. The residue was acidified with 1N aq.HCl to pH-3, extracted with EtOAc twice. The combined organic layers were washed with brine, dried over anhydrous Na₂SO₄, filtered and concentrated under reduced pressure to dryness to give (1R,19S,25S,28S,E)-17,20,23-trioxo-19-(2-phenoxyethyl)-3-oxa-18,21,24-triazatricyclo[22.2.2.0^{1,25}]octacos-7-ene-28-carboxylic acid (60 mg, yield 12.8%) as a brown oil, which was used directly in the next step. LC/MS (ESI) (m/z): 598 (M+H)⁺.

### Step 11: (1R,19S,25S,28S,E)-N-((R)-1-(4-carbamimidoylthiophen-2-yl)ethyl)-17,20,23-trioxo-19-(2-phenoxyethyl)-3-oxa-18,21,24-triazatricyclo[22.2.2.0^{1,25}]octacos-7-ene-28-carboxamide (Compound 250)

To a mixture of (1R,19S,25S,28S,E)-17,20,23-trioxo-19-(2-phenoxyethyl)-3-oxa-18,21,24-triazatricyclo[22.2.2.0^{1,25}]octacos-7-ene-28-carboxylic acid (60 mg, 0.10 mmol) and (R)-5-(1-(I²-azanyl)ethyl)thiophene-3-carboximidamide HCl salt (52 mg, 0.30 mmol) in DMF (3 mL) was added DIPEA (78 mg, 0.60 mmol) and PyBOP (80 mg, 0.15 mmol) at 0° C. under N₂ atmosphere and the mixture was stirred at 25° C. for 2 hours. The mixture was diluted with water and extracted with EtOAc twice. The combined organic layers were dried over anhydrous Na₂SO₄, filtered and concentrated under reduced pressure to dryness. The residue was purified by prep-TLC (DCM:MeOH=10:1) and prep-HPLC to give Compound 250 (1.4 mg, yield 10.0%) as a white solid. ¹H NMR (400 MHz, CD₃OD) δ 8.52 (s, 3H), 8.20 (s, 1H), 7.49 (s, 1H), 7.25-7.20 (m, 2H), 6.90 (d, J=8.2 Hz, 3H), 5.45 (d, J=27.7 Hz, 2H), 5.20 (m, 1H), 4.64 (m, 1H), 4.22 (d, J=16.2 Hz, 1H), 4.08 (d, J=16.3 Hz, 3H), 3.68-3.63 (m, 2H), 3.48 (m, 4H), 2.64 (m, 1H), 2.30 (m, 2H), 2.22 (m, 1H), 2.09 (m, 3H), 2.01 (m, 2H), 1.62 (d, J=6.0 Hz, 2H), 1.57 (d, J=6.9 Hz, 4H), 1.29 (m, 9H), 1.14 (m, 2H), 1.00 (m, 1H), 0.88 (d, J=6.4 Hz, 2H). LC/MS (ESI) (m/z): 749 (M+H)⁺.

### Step 12: 2-(tert-butyl) 3-ethyl (1S,3S,5R)-5-((pent-4-en-1-yloxy)methyl)-2-azabicyclo[3.1.0]hexane-2,3-dicarboxylate (21)

To a solution of 2-(tert-butyl) 3-ethyl (1S,3S,5R)-5-(hydroxymethyl)-2-azabicyclo [3.1.0]hexane-2,3-dicarboxylate (1.0 g, 3.51 mmol) in DCE (30 mL) was added AgOTf (2.69 g, 10.53 mmol) at 0° C. followed by 5-bromopent-1-ene (1.56 g, 10.53 mmol) and 2,6-di-tert-butylpyridine (4.02 g, 21.06 mmol) and the mixture was stirred at 30° C. for 16 hours under N₂ atmosphere. The mixture was filtered and concentrated to give the crude product which was further purified by flash chromatography (PE:EtOAc=5:1) to give 2-(tert-butyl) 3-ethyl (1S,3S,5R)-5-((pent-4-en-1-yloxy)methyl)-2-azabicyclo[3.1.0]hexane-2,3-dicarboxylate (1.0 g, Yield 83.3%) as a colorless oil. LC/MS (ESI) (m/z): 354 (M+H)⁺.

### Step 13: ethyl (1S,3S,5R)-5-((pent-4-en-1-yloxy)methyl)-2-azabicyclo[3.1.0]hexane-3-carboxylate hydrochloride (22)

A solution of 2-(tert-butyl) 3-ethyl (1 S,3S,5R)-5-((pent-4-en-1-yloxy)methyl)-2-azabicyclo [3.1.0]hexane-2,3-dicarboxylate (1.0 g, 2.82 mmol) in HCl/1,4-dioxane (10 mL) was stirred at room temperature for 1 hour. The mixture was concentrated to dryness under reduced pressure to give ethyl (1S,3S,5R)-5-((pent-4-en-1-yloxy)methyl)-2-azabicyclo[3.1.0]hexane-3-carboxylate hydrochloride (700 mg, yield 97.6%) as a colorless oil, which was directly used in the next reaction without further purification. LC/MS (ESI) m/z: 254 (M+H)⁺.

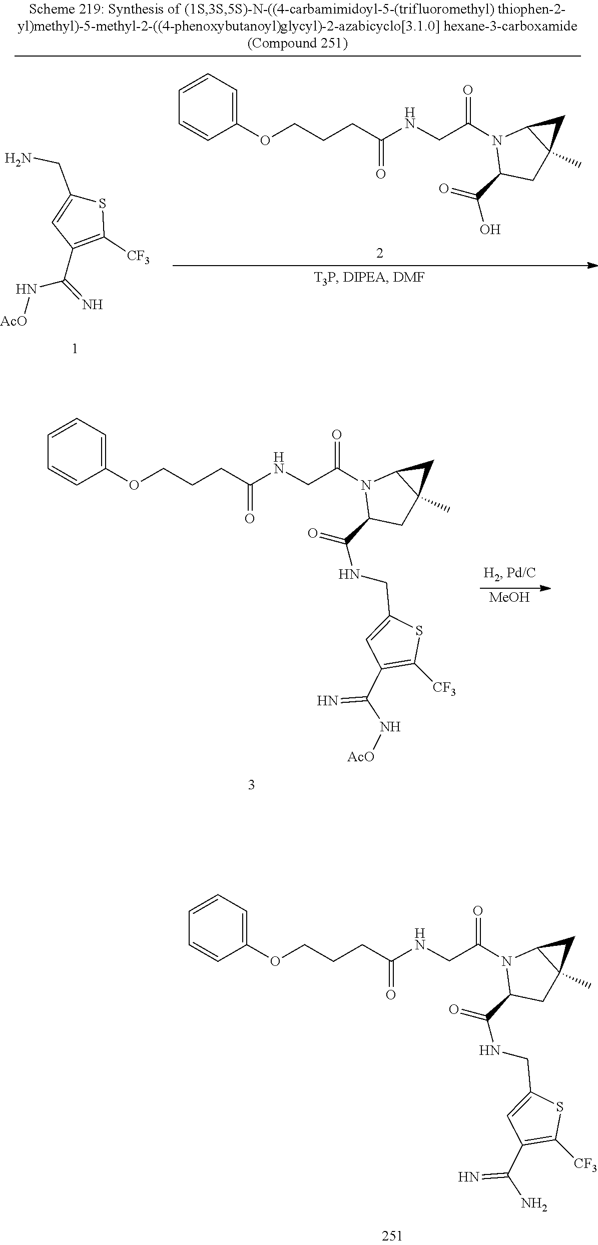 [SMILES: CC(=O)ONC(=N)c1cc(CN)sc1C(F)(F)F ; C[C@@]12C[C@@H]1N(C(=O)CNC(=O)CCCOc1ccccc1)[C@H](C(=O)O)C2 ; "MeOH" ; CC(=O)ONC(=N)c1cc(CNC(=O)[C@@H]2C[C@]3(C)C[C@@H]3N2C(=O)CNC(=O)CCCOc2ccccc2)sc1C(F)(F)F ; C[C@@]12C[C@@H]1N(C(=O)CNC(=O)CCCOc1ccccc1)[C@H](C(=O)NCc1cc(C(=N)N)c(C(F)(F)F)s1)C2]

### Step 1: (1S,3S,5S)-N-((4-(N-acetoxycarbamimidoyl)-5-(trifluoromethyl)thiophen-2-yl) methyl)-5-methyl-2-((4-phenoxybutanoyl)glycyl)-2-azabicyclo[3.1.0]hexane-3-carboxamide (3)

To a mixture of N-acetoxy-5-(aminomethyl)-2-(trifluoromethyl)thiophene-3-carboximidamide (40 mg, 0.14 mmol) and (1 S,3S,5S)-5-methyl-2-((4-phenoxybutanoyl)glycyl)-2-azabicyclo [3.1.0]hexane-3-carboxylic acid (46 mg, 0.13 mmol) in DMF (2 mL) was added DIPEA (92 mg, 0.71 mmol) and T₃P (136 mg, 0.21 mmol, 50% wt. in EtOAc) under N₂ atmosphere and the mixture was stirred at room temperature for 2 hours. The mixture was diluted with EtOAc, washed with saturated aq.NaHCO₃ solution and brine, dried over anhydrous Na₂SO₄, filtered and concentrated under reduced pressure to dryness. The residue was purified by prep-TLC (DCM:MeOH=7:1) to give (1S,3S,5S)-N-((4-(N-acetoxycarbamimidoyl)-5-(trifluoromethyl) thiophen-2-yl)methyl)-5-methyl-2-((4-phenoxybutanoyl)glycyl)-2-azabicyclo[3.1.0]hexane-3-carboxamide (15 mg, yield 18.8%) as a white solid. LC/MS (ESI) m/z: 624 (M+H)⁺.

### Step 2: (1S,3S,5S)-N-((4-carbamimidoyl-5-(trifluoromethyl)thiophen-2-yl)methyl)-5-methyl-2-((4-phenoxybutanoyl)glycyl)-2-azabicyclo[3.1.0]hexane-3-carboxamide (Compound 251)

To a mixture of (1 S,3S,5S)-N-((4-(N-acetoxycarbamimidoyl)-5-(trifluoromethyl)thiophen-2-yl) methyl)-5-methyl-2-((4-phenoxybutanoyl)glycyl)-2-azabicyclo[3.1.0]hexane-3-carboxamide (15 mg, 0.03 mmol) in MeOH (3 mL) was added Pd/C (5 mg, 10% wt.), the mixture was degassed under N₂ atmosphere for three times and stirred under a H₂ balloon at 25° C. for 15 minutes. The mixture was filtered and the filtrate was concentrated under reduced pressure to dryness. The residue was purified by prep-HPLC to give Compound 251 (1.0 mg, yield 7.4%) as a white solid. ¹H NMR (400 MHz, CD₃OD) δ 8.54 (s, 1H), 7.24 (t, J=8.0 Hz, 3H), 6.93-6.86 (m, 3H), 4.77 (d, J=3.2 Hz, 1H), 4.63-4.55 (m, 3H), 4.19 (d, J=16.5 Hz, 1H), 4.12 (d, J=16.6 Hz, 1H), 4.01 (t, J=6.2 Hz, 2H), 2.47 (t, J=7.5 Hz, 2H), 2.39 (t, J=12.4 Hz, 1H), 2.16-2.05 (m, 3H), 1.28 (s, 3H), 1.11 (dd, J=5.8, 2.1 Hz, 1H), 0.81 (t, J=5.6 Hz, 1H). LC/MS (ESI) m/z: 566 (M+H)⁺.

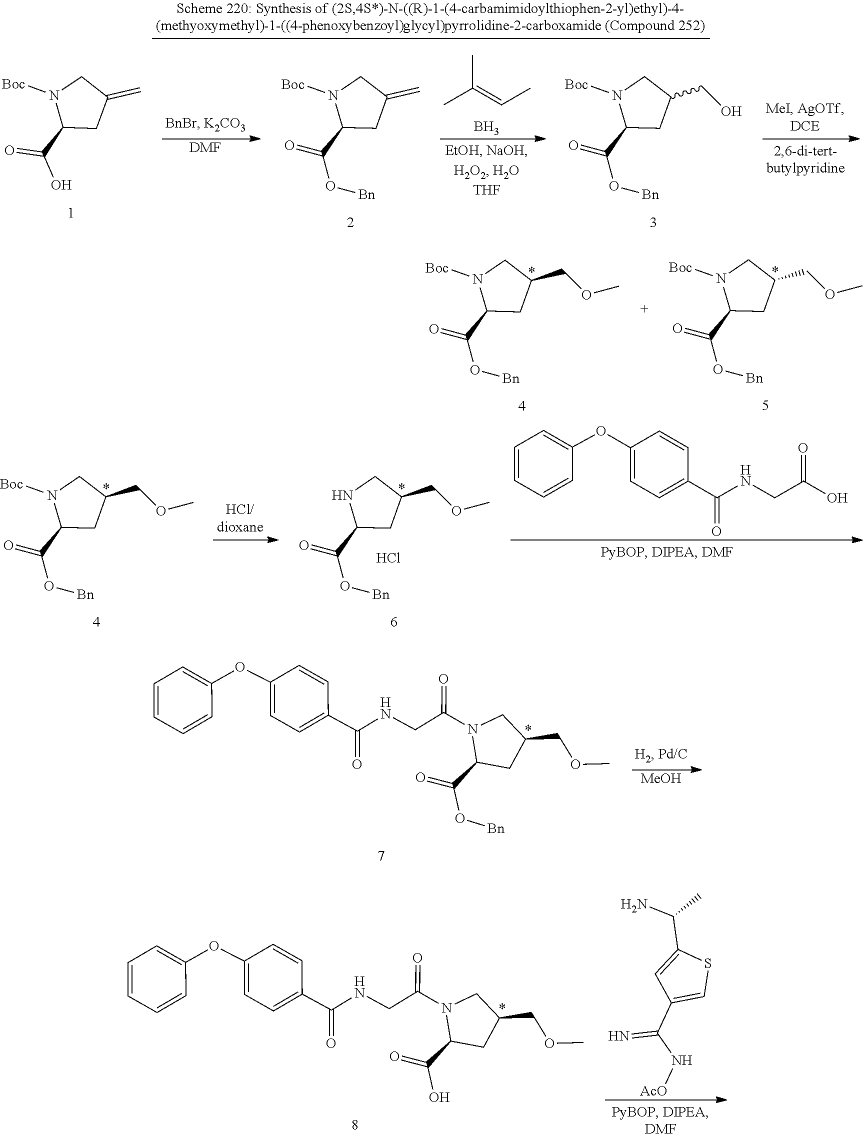

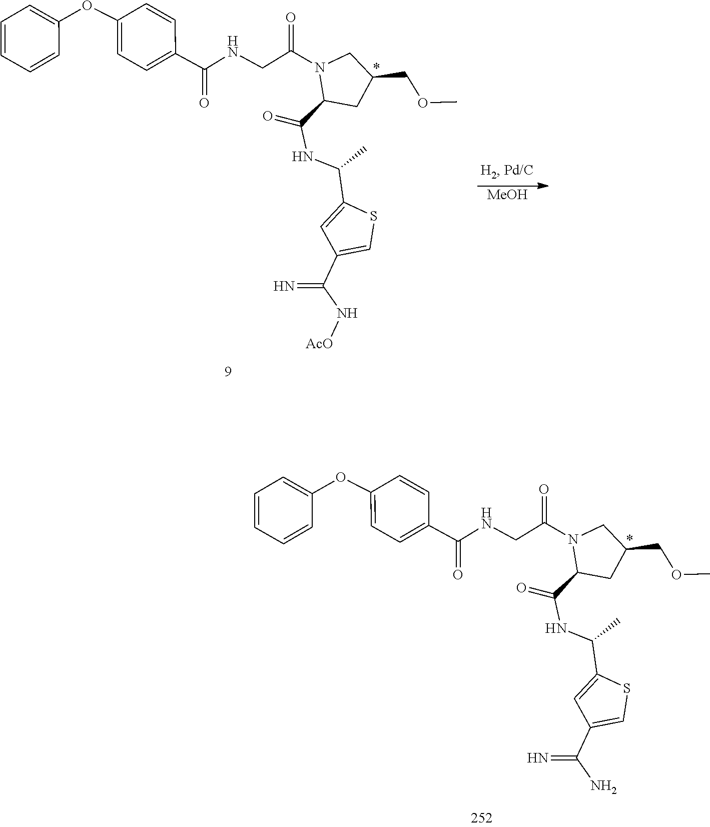 [SMILES: "MeOH" ; COC[C@H]1C[C@@H](C(=O)N[C@H](C)c2cc(C(=N)NOC(C)=O)cs2)N(C(=O)CNC(=O)c2ccc(Oc3ccccc3)cc2)C1 ; COC[C@H]1C[C@@H](C(=O)N[C@H](C)c2cc(C(=N)N)cs2)N(C(=O)CNC(=O)c2ccc(Oc3ccccc3)cc2)C1]

### Step 1: 2-benzyl 1-(tert-butyl) (S)-4-methylenepyrrolidine-1,2-dicarboxylate

To a solution of (S)-1-(tert-butoxycarbonyl)-4-methylenepyrrolidine-2-carboxylic acid (15 g, 66 mmol) in DMF (150 mL) was added K₂CO₃ (27 g, 198 mmol) and BnBr (17 g, 99 mmol) and the reaction mixture was stirred under N₂ atmosphere at room temperature overnight. The mixture was diluted with water and extracted with EtOAc twice. The combined organic layers were washed with brine, dried over anhydrous Na₂SO₄, filtered and concentrated to dryness under reduced pressure. The residue was purified by column chromatography on silica gel (PE:EtOAc=2:1) to give 2-benzyl 1-(tert-butyl) (S)-4-methylenepyrrolidine-1,2-dicarboxylate (11.2 g, yield 53.5%) as a colorless oil. LCMS (ESI) m/z=318 (M+H)⁺.

### Step 2: 2-benzyl 1-(tert-butyl) (2S)-4-(hydroxymethyl)pyrrolidine-1,2-dicarboxylate

A solution of BH₃·SMe₂ (7.3 mL, 73 mmol) in 2-methylbut-2-ene (10.2 g, 146 mmol) was stirred at 0° C. for 2 hours, and a solution of 2-benzyl 1-(tert-butyl) (S)-4-methylenepyrrolidine-1,2-dicarboxylate (11.6 g, 37 mmol) in THE (4 mL) was added at 0° C. The reaction mixture was stirred at room temperature for 20 hours, and ethanol (1.4 mL) was added and the reaction mixture was cooled down to 0° C. Rapid addition of NaOH (3M, 12 mL) was followed by cooling down the mixture to −10° C. and then slow addition of H₂O₂ (30%, 12 mL). The reaction mixture was stirred for 3 hours and the aqueous and organic layers were separated. The aqueous layer was extracted with MTBE twice. The combined organic layers were washed with brine, dried over anhydrous Na₂SO₄, filtered and concentrated to dryness under reduced pressure. The residue was purified by column chromatography on silica gel (PE:EtOAc=3:1) to give 2-benzyl 1-(tert-butyl) (2S)-4-(hydroxymethyl)pyrrolidine-1,2-dicarboxylate (6.6 g, yield 53.8%) as a colorless oil. LCMS (ESI) m/z=336 (M+H)⁺.

### Step 3: 2-benzyl 1-(tert-butyl) (2S,4S*)-4-(methoxymethyl)pyrrolidine-1,2-dicarboxylate and 2-benzyl 1-(tert-butyl) (2S,4R*)-4-(methoxymethyl)pyrrolidine-1,2-dicarboxylate

To a solution of 2-benzyl 1-(tert-butyl) (2S)-4-(hydroxymethyl)pyrrolidine-1,2-dicarboxylate (2.0 g, 6.0 mmol) in DCE (30 mL) was added AgOTf (3.0 g, 12 mmol) and iodomethane (2.8 g, 18 mmol) under N₂ atmosphere at room temperature and the reaction mixture was stirred overnight. The mixture was diluted with water and extracted with EtOAc twice. The combined organic layers were washed with brine, dried over anhydrous Na₂SO₄, filtered and concentrated to dryness under reduced pressure. The residue was purified by column chromatography on silica gel (PE:EtOAc=4:1) and SFC to give 2-benzyl 1-(tert-butyl) (2S,4S*)-4-(methoxymethyl)pyrrolidine-1,2-dicarboxylate (350 mg, yield 16.7%) as a colorless oil. LCMS (ESI) m/z=350 (M+H)⁺ and 2-benzyl 1-(tert-butyl) (2S,4R*)-4-(methoxymethyl)pyrrolidine-1,2-dicarboxylate (110 mg, yield 5.2%) as colorless oil. LCMS (ESI) m/z=350 (M+H)⁺.

### Step 4: benzyl (2S,4S*)-4-(methoxymethyl)pyrrolidine-2-carboxylate hydrochloride

To a solution of 2-benzyl 1-(tert-butyl) (2S,4S*)-4-(methoxymethyl)pyrrolidine-1,2-dicarboxylate (350 mg, 1.0 mmol) in HCl/1,4-dioxane (4 mL) was stirred under N₂ atmosphere at room temperature for 30 minutes. The reaction mixture was concentrated to dryness under reduced pressure, diluted with DCM again and dried under vacuum to give benzyl (2S,4S*)-4-(methoxymethyl)pyrrolidine-2-carboxylate hydrochloride (250 mg, yield 99.7%) which was used directly in the next step without further purification. LC/MS (ESI) m/z: 250 (M+H)⁺.

### Step 5: benzyl (2S,4S*)-4-(methoxymethyl)-1-((4-phenoxybenzoyl)glycyl)pyrrolidine-2-carboxylate

To a mixture of benzyl (2S,4S*)-4-(methoxymethyl)pyrrolidine-2-carboxylate hydrochloride (40 mg, 0.16 mmol) and (4-phenoxybenzoyl)glycine (43 mg, 0.16 mmol) in DMF (1 mL) was added DIPEA (124 mg, 0.96 mmol) and PyBOP (100 mg, 0.2 mmol) at 0° C. under N₂ atmosphere and the mixture was stirred at 25° C. for 1 hour. The reaction mixture was diluted with water and extracted with EtOAc twice. The combined organic layers were washed with brine, dried over anhydrous Na₂SO₄, filtered and concentrated to dryness under reduced pressure. The residue was purified by column chromatography on silica gel (PE:EtOAc=2:1) to give methyl (2S,4S*)-1-((4-(4-fluorophenoxy)benzoyl)glycyl)-4-(methoxymethyl)pyrrolidine-2-carboxylate (60 mg, yield 74.7%) as a colorless oil. LC/MS (ESI) m/z: 503 (M+H)⁺.

### Step 6: (2S,4S*)-4-(methoxymethyl)-1-((4-phenoxybenzoyl)glycyl)pyrrolidine-2-carboxylic acid

To a solution of benzyl (2S,4S*)-4-(methoxymethyl)-1-((4-phenoxybenzoyl)glycyl)pyrrolidine-2-carboxylate (55 mg, 1.1 mmol) in MeOH (1 mL) was added Pd/C (20 mg, 10% wt.) at room temperature and the mixture was stirred under H₂ atmosphere at room temperature for 2 hours. The mixture was filtered and the filtrate was concentrated to dryness under reduced pressure to give (2S,4S)-4-(methoxymethyl)-1-((4-phenoxybenzoyl)glycyl)pyrrolidine-2-carboxylic acid (40 mg, yield 88.3%) as a colorless oil which was used directly in the next step. LC/MS (ESI) (m/z): 413 (M+H)⁺.

### Step 7: (2S,4S*)-N-((R)-1-(4-(N-acetoxycarbamimidoyl)thiophen-2-yl)ethyl)-4-(methoxymethyl)-1-((4-phenoxybenzoyl)glycyl)pyrrolidine-2-carboxamide

To a mixture of (2S,4S*)-4-(methoxymethyl)-1-((4-phenoxybenzoyl)glycyl)pyrrolidine-2-carboxylic acid (40 mg, 0.097 mmol) and (R)-N-acetoxy-5-(1-aminoethyl)thiophene-3-carboximidamide (55 mg, 0.24 mmol) in DMF (1 mL) was added DIPEA (93 mg, 0.72 mmol) and PyBOP (83 mg, 0.16 mmol) at 0° C. under N₂ atmosphere and the mixture was stirred at 25° C. for 1 hour. The reaction mixture was diluted with water and extracted with EtOAc twice. The combined organic layers were washed with brine, dried over anhydrous Na₂SO₄, filtered and concentrated to dryness under reduced pressure. The residue was purified by column chromatography on silica gel (PE:EtOAc=2:1) to give (2S,4S*)-N-((R)-1-(4-(N-acetoxycarbamimidoyl)thiophen-2-yl)ethyl)-4-(methoxymethyl)-1-((4-phenoxybenzoyl)glycyl)pyrrolidine-2-carboxamide (23 mg, yield 38.2%) as a yellow solid. LC/MS (ESI) m/z: 622 (M+H)⁺.

### Step 8: (2S,4S*)-N-((R)-1-(4-carbamimidoylthiophen-2-yl)ethyl)-4-(methoxymethyl)-1-((4-phenoxybenzoyl)glycyl)pyrrolidine-2-carboxamide (Compound 252)

To a solution of (2S,4S)-N-((R)-1-(4-(N-acetoxycarbamimidoyl)thiophen-2-yl)ethyl)-4-(methoxymethyl)-1-((4-phenoxybenzoyl)glycyl)pyrrolidine-2-carboxamide (23 mg, 0.037 mmol) in MeOH (1 mL) was added Pd/C (10 mg, 10% wt.) at room temperature and the mixture was stirred under H₂ atmosphere at room temperature for 2 hours. The mixture was filtered and the filtrate was concentrated to dryness under reduced pressure. The residue was purified by prep-HPLC to give Compound 252 (6 mg, yield 28.8%) as a white solid. ¹H NMR (400 MHz, CD₃OD) δ 8.55 (s, 1H), 8.23 (d, J=10.2 Hz, 1H), 7.85 (d, J=8.7 Hz, 2H), 7.53 (s, 1H), 7.41 (t, J=7.9 Hz, 2H), 7.21 (d, J=7.4 Hz, 1H), 7.06 (d, J=7.7 Hz, 2H), 7.00 (d, J=8.7 Hz, 2H), 5.26 (q, J=6.5 Hz, 1H), 4.43 (t, J=8.2 Hz, 1H), 4.27 (d, J=16.6 Hz, 1H), 4.10 (d, J=16.6 Hz, 1H), 3.97-3.91 (m, 1H), 3.43 (dt, J=10.0, 7.9 Hz, 3H), 3.34 (d, J=7.5 Hz, 3H), 2.68-2.60 (m, 1H), 2.46-2.37 (m, 1H), 1.76 (dd, J=15.4, 5.9 Hz, 1H), 1.60 (t, J=15.0 Hz, 3H). LCMS (ESI) (m/z): 564 (M+H)⁺.

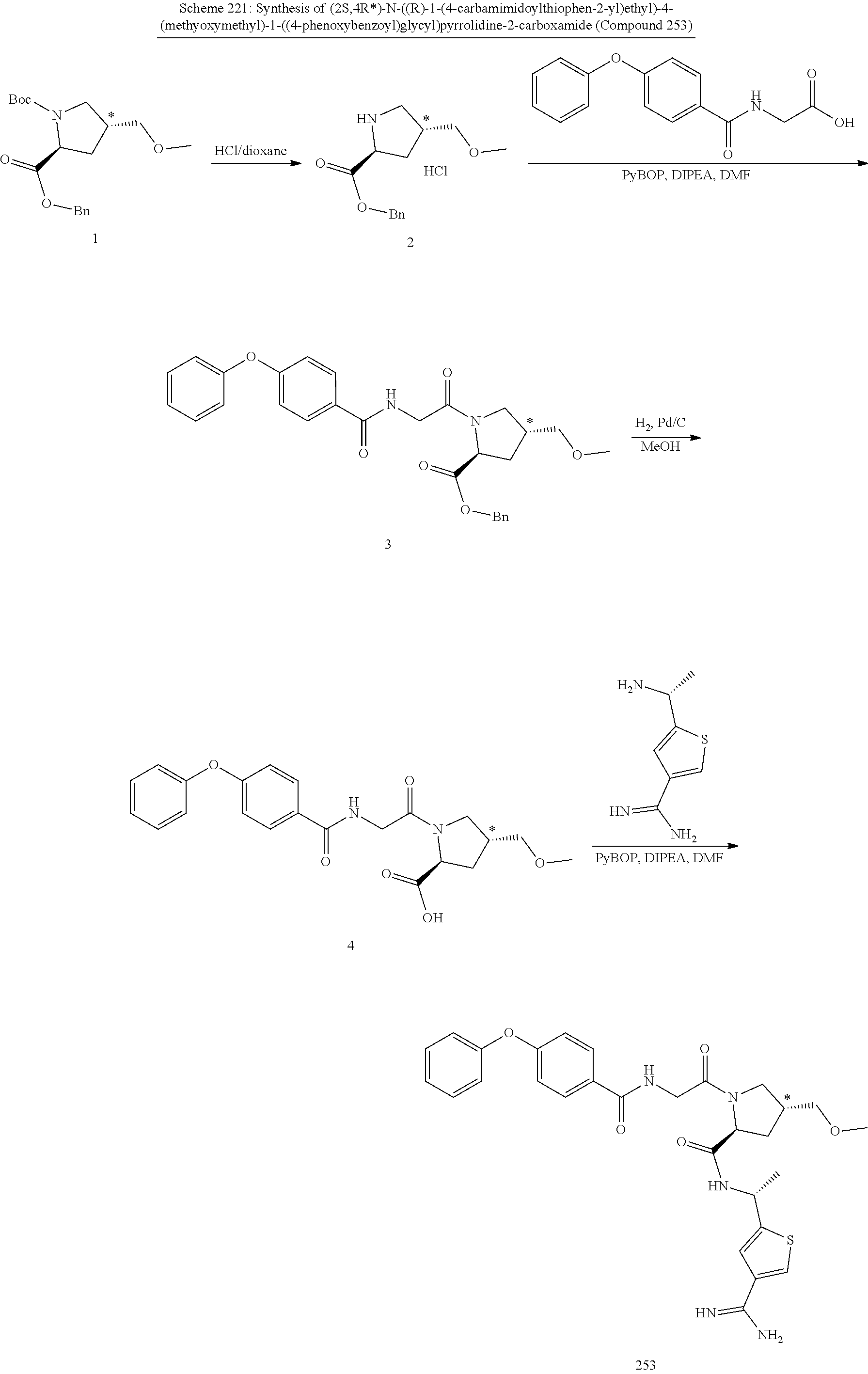

### Step 1: benzyl (2S,4R*)-4-(methoxymethyl)pyrrolidine-2-carboxylate hydrochloride

To a mixture of 2-benzyl 1-(tert-butyl) (2S,4R*)-4-(methoxymethyl)pyrrolidine-1,2-dicarboxylate (110 mg, 0.31 mmol) in HCl/1,4-dioxane (1.0 mL) was stirred under N₂ atmosphere at room temperature for 30 minutes. The reaction mixture was concentrated to dryness under reduced pressure, diluted with DCM again and concentrated to dryness under reduced pressure to give benzyl (2S,4R*)-4-(methoxymethyl)pyrrolidine-2-carboxylate hydrochloride (75 mg, yield 86.2%) which was used directly in the next step without further purification. LC/MS (ESI) m/z: 250 (M+H)⁺.

### Step 2: benzyl (2S,4R*)-4-(methoxymethyl)-1-((4-phenoxybenzoyl)glycyl)pyrrolidine-2-carboxylate

To a mixture of benzyl (2S,4R)-4-(methoxymethyl)pyrrolidine-2-carboxylate (43 mg, 0.16 mmol) and (4-phenoxybenzoyl)glycine (43 mg, 0.16 mmol) in DMF (2.0 mL) was added PyBOP (109 mg, 0.21 mmol) and DIPEA (109 mg, 0.96 mmol) under N₂ atmosphere at room temperature and stirred for 1 hour. The reaction mixture was diluted with water and extracted with EtOAc twice. The combined organic layers were washed with brine, dried over anhydrous Na₂SO₄, filtered and concentrated to dryness under reduced pressure. The residue was purified by column chromatography on silica gel (PE:EtOAc=2:1) to give benzyl (2S,4R)-4-(methoxymethyl)-1-((4-phenoxybenzoyl)glycyl)pyrrolidine-2-carboxylate (60 mg, yield 74.7%) as a yellow oil. LCMS (ESI) m/z=503 (M+H)⁺.

### Step 3: (2S,4R*)-4-(methoxymethyl)-1-((4-phenoxybenzoyl)glycyl)pyrrolidine-2-carboxylic acid

To a solution of benzyl (2S,4R*)-4-(methoxymethyl)-1-((4-phenoxybenzoyl)glycyl)pyrrolidine-2-carboxylate (60 mg, 0.11 mmol) in MeOH (5 mL) was added Pd/C (20 mg, 10% wt.) at room temperature and the mixture was stirred under H₂ atmosphere at room temperature overnight. The mixture was filtered and the filtrate was concentrated to dryness under reduced pressure to give (2S,4R)-4-(methoxymethyl)-1-((4-phenoxybenzoyl)glycyl) pyrrolidine-2-carboxylic acid (40 mg, yield 88.3%) as a colorless oil, which was used directly in the next step. LC/MS (ESI) (m/z): 413 (M+H)⁺.

### Step 4: (2S,4R)-N-((R)-1-(4-carbamimidoylthiophen-2-yl)ethyl)-4-(methoxymethyl)-1-((4-phenoxybenzoyl)glycyl)pyrrolidine-2-carboxamide (Compound 253)

To a mixture of (2S,4R*)-4-(methoxymethyl)-1-((4-phenoxybenzoyl)glycyl)pyrrolidine-2-carboxylic acid (20 mg, 0.048 mmol) and (R)-5-(1-aminoethyl)thiophene-3-carboximidamide (16 mg, 0.096 mmol) in DMF (1 mL) was added DIPEA (37 mg, 0.29 mmol) and PyBOP (32 mg, 0.062 mmol) at 0° C. and the mixture was stirred under N₂ atmosphere at room temperature for 1 hour. The organic layer was diluted with water and extracted with EtOAc twice. The combined organic layers were washed with brine, dried over anhydrous Na₂SO₄, filtered and concentrated to dryness under reduced pressure. The residue was purified by flash chromatography on silica gel (DCM:MeOH=8:1) and prep-HPLC to give Compound 253 (5 mg, yield 18.5%) as a white solid. ¹H NMR (400 MHz, CD₃OD) δ 8.54 (s, 1H), 8.23 (d, J=7.2 Hz, 1H), 7.85 (d, J=8.7 Hz, 2H), 7.50 (d, J=14.2 Hz, 1H), 7.41 (t, J=8.0 Hz, 2H), 7.20 (t, J=7.3 Hz, 1H), 7.06 (d, J=7.8 Hz, 2H), 7.00 (d, J=8.8 Hz, 2H), 5.26 (d, J=6.7 Hz, 1H), 4.56-4.52 (m, 1H), 4.18 (s, 2H), 3.91-3.82 (m, 1H), 3.49-3.40 (m, 3H), 3.36 (s, 3H), 2.80-2.58 (m, 1H), 2.07 (dd, J=18.6, 9.7 Hz, 2H), 1.60 (t, J=15.6 Hz, 3H). LC/MS (ESI) (m/z): 564 (M+H)⁺.

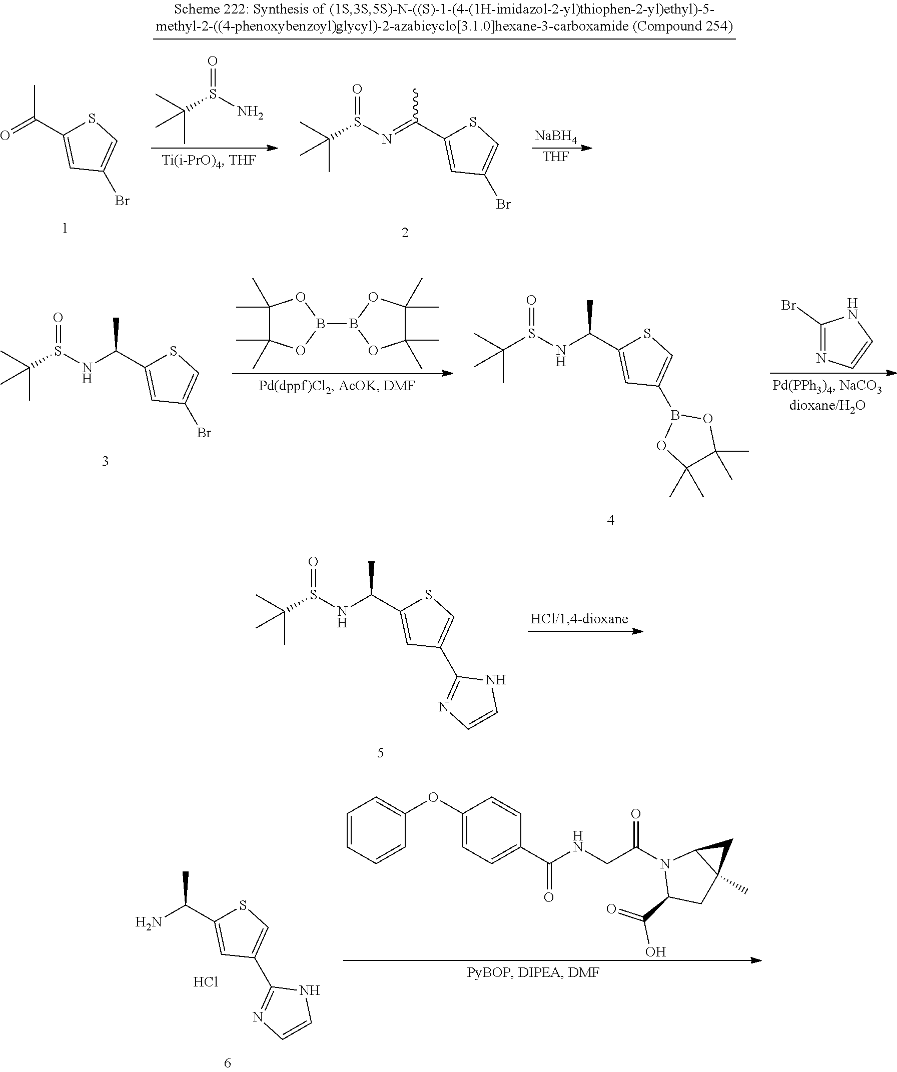

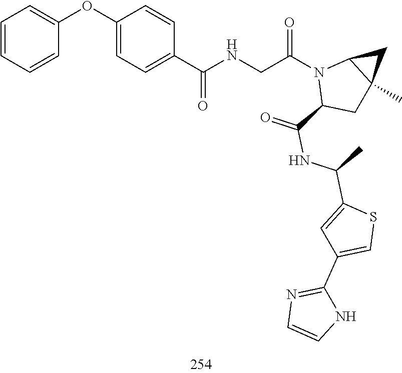 [SMILES: C[C@H](NC(=O)[C@@H]1C[C@]2(C)C[C@@H]2N1C(=O)CNC(=O)c1ccc(Oc2ccccc2)cc1)c1cc(-c2ncc[nH]2)cs1]

### Step 1: (S)-N-(1-(4-bromothiophen-2-yl)ethylidene)-2-methylpropane-2-sulfinamide

To a solution of 1-(4-bromothiophen-2-yl)ethan-1-one (3 g, 15 mmol) in THE (30 mL) was added (S)-2-methylpropane-2-sulfinamide (1.8 g, 15 mmol) and Ti(iPrO)₄ (9.6 g, 34 mmol) under N₂ atmosphere at 85° C. and the reaction mixture was stirred for 4 hours. The mixture was diluted with water and extracted with EtOAc twice. The combined organic layers were washed with brine, dried over anhydrous Na₂SO₄, filtered and concentrated to dryness under reduced pressure. The residue was purified by column chromatography on silica gel (PE:EtOAc=4:1) to give (S)-N-(1-(4-bromothiophen-2-yl)ethylidene)-2-methylpropane-2-sulfinamide (2.8 g, yield 61.9%) as a white solid. LCMS (ESI) m/z=310 (M+H)⁺.

### Step 2: (S)-N-((S)-1-(4-bromothiophen-2-yl)ethyl)-2-methylpropane-2-sulfinamide

To a solution of (S)-N-(1-(4-bromothiophen-2-yl)ethylidene)-2-methylpropane-2-sulfinamide (2.8 g, 9.1 mmol) in THE (30 mL) was added NaBH₄ (516 mg, 14 mmol) in portions under N₂ atmosphere at room temperature and stirred for 30 minutes. The mixture was diluted with water and extracted with EtOAc twice. The combined organic layers were washed with brine, dried over anhydrous Na₂SO₄, filtered and concentrated to dryness under reduced pressure. The residue was purified by column chromatography on silica gel (PE:EtOAc=2:1) and recrystallized with EtOAc to give (S)-N-((S)-1-(4-bromothiophen-2-yl)ethyl)-2-methylpropane-2-sulfinamide (700 mg, yield 24.9%) as a white solid. LCMS (ESI) m/z=310 (M+H)⁺.

### Step 3: (S)-2-methyl-N-((S)-1-(4-(4,4,5,5-tetramethyl-1,3,2-dioxaborolan-2-yl)thiophen-2-yl)ethyl)propane-2-sulfinamide

To a mixture of (S)-N-((S)-1-(4-bromothiophen-2-yl)ethyl)-2-methylpropane-2-sulfinamide (300 mg, 0.97 mmol) and Pd(dppf)Cl₂ (71 mg, 0.097 mmol) in 1,4-dioxane (5 mL) was added Pin₂B₂ (480 mg, 1.9 mmol) and CH₃COOK (380 mg, 3.9 mmol) at 0° C. The mixture was degassed under N₂ atmosphere and the reaction mixture was stirred at 85° C. for 2 hours. The mixture was used directly in next step without purification.

### Step 4: (S)-N-((S)-1-(4-(1H-imidazol-2-yl)thiophen-2-yl)ethyl)-2-methylpropane-2-sulfinamide

The mixture of previous step was added 2-bromo-1H-imidazole (283 mg, 1.9 mmol), Pd(PPh₃)₄ (112 mg, 0.097 mmol), Na₂CO₃ (413 mg, 3.9 mmol), 1,4-dioxane (1 mL) and water (3 mL). The mixture was degassed under N₂ atmosphere and stirred at 85° C. for 4 hours. The mixture was diluted with water and extracted with EtOAc twice. The combined organic layers were washed with brine, dried over anhydrous Na₂SO₄, filtered and concentrated under reduced pressure to dryness. The residue was purified by column chromatography on silica gel (PE:EtOAc=5:1) to give (S)-N-((S)-1-(4-(1H-imidazol-2-yl)thiophen-2-yl)ethyl)-2-methylpropane-2-sulfinamide (220 mg, two step yield 76.4%) as a colorless oil. LCMS (ESI) m/z=298 (M+H)⁺.

### Step 5: (S)-1-(4-(1H-imidazol-2-yl)thiophen-2-yl)ethan-1-amine hydrochloride

To a solution of (S)-N-((S)-1-(4-(1H-imidazol-2-yl)thiophen-2-yl)ethyl)-2-methylpropane-2-sulfinamide (100 mg, 0.34 mmol) in HCl/1,4-dioxane (1 mL) was stirred under N₂ atmosphere at room temperature for 10 minutes. The reaction mixture was concentrated to dryness under reduced pressure, diluted with DCM again and dried under vacuum to give (S)-1-(4-(1H-imidazol-2-yl)thiophen-2-yl)ethan-1-amine hydrochloride (65 mg, yield 83.1%) which was used directly in the next step without further purification. LC/MS (ESI) m/z: 194 (M+H)⁺.

### Step 6: (1S,3S,5S)-N-((S)-1-(4-(1H-imidazol-2-yl)thiophen-2-yl)ethyl)-5-methyl-2-((4-phenoxybenzoyl)glycyl)-2-azabicyclo[3.1.0]hexane-3-carboxamide (Compound 254)

To a mixture of (S)-1-(4-(1H-imidazol-2-yl)thiophen-2-yl)ethan-1-amine (65 mg, 0.34 mmol) and (1S,3S,5S)-5-methyl-2-((4-phenoxybenzoyl)glycyl)-2-azabicyclo[3.1.0]hexane-3-carboxylic acid (134 mg, 0.34 mmol) in DMF (2 mL) was added DIPEA (263 mg, 2.0 mmol) and PyBOP (194 mg, 0.37 mmol) at 0° C. and the mixture was stirred under N₂ atmosphere at room temperature for 2 hours. The organic layer was diluted with water and extracted with EtOAc twice. The combined organic layers were washed with brine, dried over anhydrous Na₂SO₄, filtered and concentrated to dryness under reduced pressure. The residue was purified by flash chromatography on silica gel (DCM:MeOH=9:1) to give Compound 254 (15 mg, yield 7.7%) as a white solid. ¹H NMR (400 MHz, CD₃OD) δ 8.19 (s, 1H), 7.80 (t, J=13.5 Hz, 3H), 7.40 (dd, J=20.8, 12.5 Hz, 3H), 7.25-7.18 (m, 3H), 7.04 (d, J=7.7 Hz, 2H), 6.92 (d, J=8.8 Hz, 2H), 5.28 (d, J=6.5 Hz, 1H), 4.83-4.82 (m, 1H), 4.32 (s, 2H), 3.42 (d, J=3.6 Hz, 1H), 2.43 (t, J=12.5 Hz, 1H), 2.20 (dd, J=13.3, 3.1 Hz, 1H), 1.63 (d, J=7.0 Hz, 3H), 1.30 (s, 3H), 1.19 (d, J=3.4 Hz, 1H), 0.83 (d, J=5.5 Hz, 1H). LC/MS (ESI) (m/z): 570 (M+H)⁺.

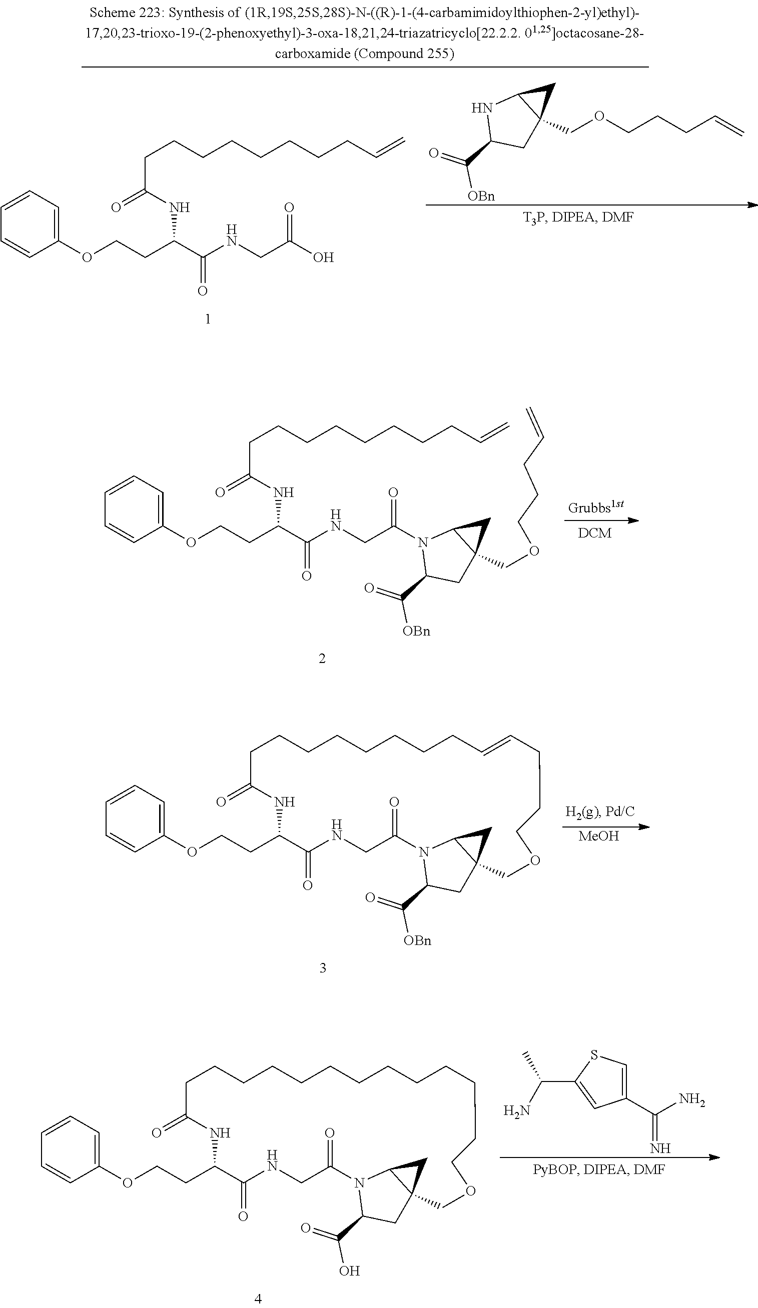

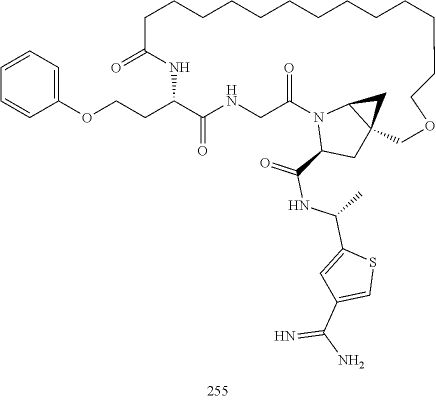 [SMILES: C[C@@H](NC(=O)[C@@H]1C[C@@]23COCCCCCCCCCCCCCC(=O)N[C@@H](CCOc4ccccc4)C(=O)NCC(=O)N1[C@H]2C3)c1cc(C(=N)N)cs1]

### Step 1: benzyl (1S,3S,5R)-5-((pent-4-en-1-yloxy)methyl)-2-(O-phenyl-N-(undec-10-enoyl)-L-homoserylglycyl)-2-azabicyclo[3.1.0]hexane-3-carboxylate (2)

To a mixture of O-phenyl-N-(undec-10-enoyl)-L-homoserylglycine (1.02 g, 2.44 mmol) and benzyl (1S,3S,5R)-5-((pent-4-en-1-yloxy)methyl)-2-azabicyclo[3.1.0]hexane-3-carboxylate HCl salt (830 mg, 2.68 mmol) in DMF (10 mL) was added DIPEA (2.0 mL, 12.20 mmol) and T₃P (4.6 g, 7.32 mmol, 50% wt. in EtOAc) under N₂ atmosphere and the reaction mixture was stirred at room temperature for 2 hours. The mixture was diluted with EtOAc, washed with saturated aq.NaHCO₃ and brine, dried over anhydrous Na₂SO₄, filtered and concentrated under reduced pressure to dryness. The residue was purified by column chromatography on silica gel (DCM:MeOH=97:3) to give benzyl (1S,3S,5R)-5-((pent-4-en-1-yloxy)methyl)-2-(O-phenyl-N-(undec-10-enoyl)-L-homoserylglycyl)-2-azabicyclo[3.1.0]hexane-3-carboxylate (1.1 g, yield 63.2%) as a brown oil. LC/MS (ESI) m/z: 716 (M+H)⁺.

### Step 2: benzyl (1R,19S,25S,28S,E)-17,20,23-trioxo-19-(2-phenoxyethyl)-3-oxa-18,21,24-triazatricyclo[22.2.2.0^{1,25}]octacos-7-ene-28-carboxylate (3)

To a solution of benzyl (1S,3S,5R)-5-((pent-4-en-1-yloxy)methyl)-2-(O-phenyl-N-(undec-10-enoyl)-L-homoserylglycyl)-2-azabicyclo[3.1.0]hexane-3-carboxylate (600 mg, 0.84 mmol) in dry DCM (60 mL) was added Grubbs 1^{st} catalyst (172 mg, 0.21 mmol) and the mixture was stirred at 28° C. for 16 hours under N₂ atmosphere. The mixture was concentrated to dryness under reduced pressure and the residue was purified by flash chromatography (DCM:MeOH=98:2) to give benzyl (1R,19S,25S,28S,E)-17,20,23-trioxo-19-(2-phenoxyethyl)-3-oxa-18,21,24-triazatricyclo[22.2.2.0^{1,25}]octacos-7-ene-28-carboxylate (250 mg, yield 43.4%) as a black oil. LC/MS (ESI) m/z: 688 (M+H)⁺.

### Step 3: (1R,19S,25S,28S)-17,20,23-trioxo-19-(2-phenoxyethyl)-3-oxa-18,21,24-triazatricyclo [22.2.2.0^{1,25}]octacosane-28-carboxylic acid (4)

To a solution of benzyl (1R,19S,25S,28S,E)-17,20,23-trioxo-19-(2-phenoxyethyl)-3-oxa-18,21,24-triazatricyclo[22.2.2.0^{1,25}]octacos-7-ene-28-carboxylate (65 mg, 0.09 mmol) in MeOH (5 mL) was added Pd/C (10 mg, 10% wt.), and the reaction mixture was stirred at 25° C. under H₂ atmosphere for 2 hours. The mixture was filtered and concentrated to give (1R,19S,25S,28S)-17,20,23-trioxo-19-(2-phenoxyethyl)-3-oxa-18,21,24-triazatricyclo[22.2.2.0^{1,25}]octacosane-28-carboxylic acid (56 mg, yield 98.2%) as a brown solid. LC/MS (ESI) m/z: 600 (M+H)⁺.

### Step 4: (1R,19S,25S,28S)-N-((R)-1-(4-carbamimidoylthiophen-2-yl)ethyl)-17,20,23-trioxo-19-(2-phenoxyethyl)-3-oxa-18,21,24-triazatricyclo[22.2.2.0¹²⁵]octacosane-28-carboxamide (Compound 255)

To a mixture of (1R,19S,25S,28S)-17,20,23-trioxo-19-(2-phenoxyethyl)-3-oxa-18,21,24-triazatricyclo[22.2.2.0^{1,25}]octacosane-28-carboxylic acid (57 mg, 0.10 mmol) and (R)-5-(1-(I2-azanyl)ethyl)thiophene-3-carboximidamide HCl salt (48 mg, 0.30 mmol) in DMF (3 mL) was added DIPEA (74 mg, 0.60 mmol) and PyBOP (74 mg, 0.15 mmol) at 0° C. under N₂ atmosphere and the mixture was stirred at 25° C. for 2 hours. The mixture was diluted with water and extracted with EtOAc twice. The combined organic layers were dried over anhydrous Na₂SO₄, filtered and concentrated under reduced pressure to dryness. The residue was purified by prep-TLC (DCM:MeOH=10:1) and prep-HPLC to give Compound 255 (8 mg, yield 11.1%) as a white solid. ¹H NMR (400 MHz, CD₃OD) δ 8.50 (s, 1H), 8.20 (d, J=1.4 Hz, 1H), 7.47 (s, 1H), 7.22 (t, J=8.0 Hz, 2H), 6.90 (d, J=8.0 Hz, 3H), 5.29-5.16 (m, 1H), 4.80 (s, 1H), 4.62 (dd, J=8.7, 5.7 Hz, 1H), 4.35 (d, J=16.6 Hz, 1H), 4.12-3.98 (m, 3H), 3.73 (d, J=10.3 Hz, 1H), 3.49 (d, J=4.7 Hz, 3H), 3.26 (d, J=10.4 Hz, 1H), 2.64 (t, J=12.7 Hz, 1H), 2.37-2.21 (m, 3H), 2.14-2.02 (m, 2H), 1.71 (s, 1H), 1.56 (d, J=6.9 Hz, 5H), 1.34 (d, J=35.3 Hz, 18H), 1.03 (t, J=5.6 Hz, 1H). LC/MS (ESI) (m/z): 751 (M+H)⁺.

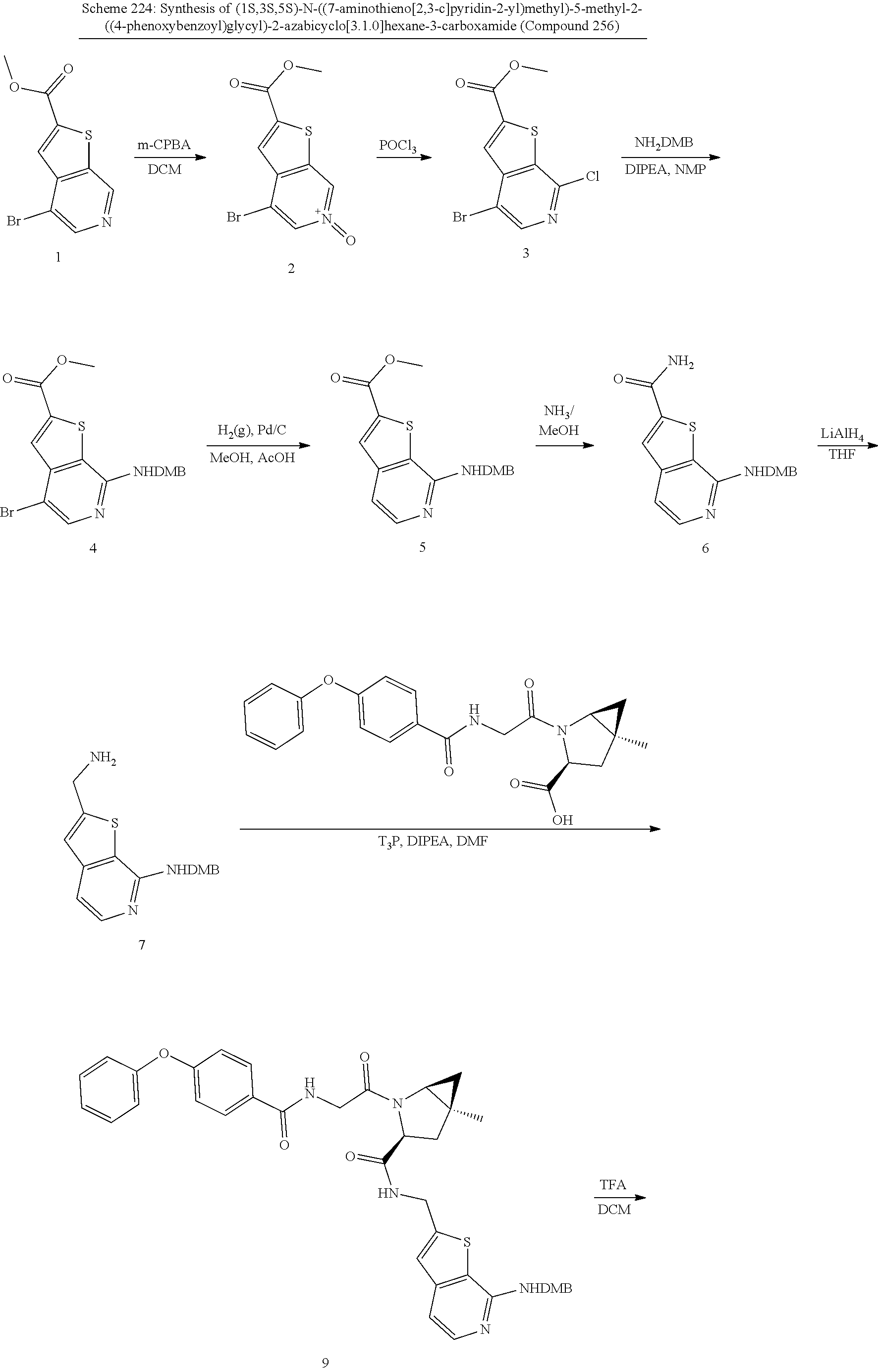

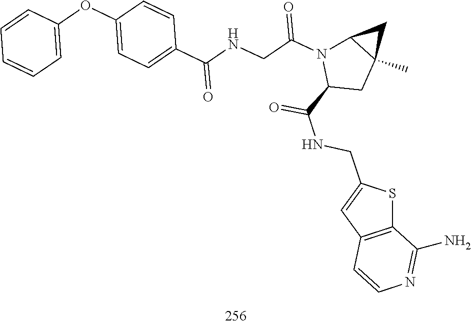 [SMILES: C[C@@]12C[C@@H]1N(C(=O)CNC(=O)c1ccc(Oc3ccccc3)cc1)[C@H](C(=O)NCc1cc3ccnc(N)c3s1)C2]

### Step 1: 4-bromo-2-(methoxycarbonyl)thieno[2,3-c]pyridine 6-oxide (2)

To a solution of methyl 4-bromothieno[2,3-c]pyridine-2-carboxylate (1.0 g, 3.69 mmol) in DCM (33 mL) was added m-CPBA (1.07 g, 6.27 mmol) at 0° C. and the reaction mixture was stirred at 25° C. for 4 hours. The mixture was filtered and the filtrate was washed with saturated aq.NaHCO₃ solution and water three times. The organic layers were dried over anhydrous Na₂SO₄, filtered and concentrated under reduced pressure to give 4-bromo-2-(methoxycarbonyl)thieno[2,3-c]pyridine 6-oxide (570 mg, yield 53.8%) as a yellow solid. LC/MS (ESI) m/z: 288 (M+H)⁺.

### Step 2: methyl 4-bromo-7-chlorothieno[2,3-c]pyridine-2-carboxylate (3)

To a solution of 4-bromo-2-(methoxycarbonyl)thieno[2,3-c]pyridine 6-oxide (570 mg, 1.98 mmol) in POCl₃ (6 mL) and the mixture was stirred at 40° C. for 2 hours. The mixture was concentrated and diluted with DCM and washed with saturated aq.NaHCO₃ solution twice. The combined organic layers were dried over anhydrous Na₂SO₄, filtered and concentrated under reduced pressure to dryness.

The residue was purified by column chromatography on silica gel (PE:DCM=5:1) to give methyl 4-bromo-7-chlorothieno[2,3-c]pyridine-2-carboxylate (300 mg, yield 49.7%) as a white solid. LC/MS (ESI) m/z: 306 (M+H)⁺.

### Step 3: methyl 4-bromo-7-((2,4-dimethoxybenzyl)amino)thieno[2,3-c]pyridine-2-carboxylate (4)

To a mixture of methyl 4-bromo-7-chlorothieno[2,3-c]pyridine-2-carboxylate (300 mg, 0.98 mmol) and (2,4-dimethoxyphenyl)methanamine (247 mg, 1.47 mmol) in NMP (5.0 mL) was added DIPEA (0.49 mL, 2.94 mmol) and the mixture was stirred at 100° C. for 16 hours. The mixture was diluted with H₂O and extracted with EtOAc twice. The combined organic layers were dried over anhydrous Na₂SO₄, filtered and concentrated to dryness under reduced pressure. The residue was purified by column chromatography on silica gel (PE:EtOAc=5:1) to give methyl 4-bromo-7-((2,4-dimethoxybenzyl)amino)thieno[2,3-c]pyridine-2-carboxylate (64 mg, yield 14.9%) as a yellow solid. LC/MS (ESI) m/z: 437/439 (M+H)⁺.

### Step 4: methyl 7-((2,4-dimethoxybenzyl)amino)thieno[2,3-c]pyridine-2-carboxylate (5)

To a solution of methyl 4-bromo-7-((2,4-dimethoxybenzyl)amino)thieno[2,3-c]pyridine-2-carboxylate (64 mg, 0.15 mmol) in MeOH (2 mL) and AcOH (0.5 mL) was added Pd/C (10 mg, 10% wt.) and the reaction mixture was stirred at 25° C. under H₂ atmosphere for 2 hours. The mixture was filtered and concentrated to give methyl 7-((2,4-dimethoxybenzyl)amino)thieno[2,3-c]pyridine-2-carboxylate (46 g, yield 86.8%) as a yellow solid. LC/MS (ESI) m/z: 359 (M+H)⁺.

### Step 5: 7-((2,4-dimethoxybenzyl)amino)thieno[2,3-c]pyridine-2-carboxamide (6)

To a solution of methyl 7-((2,4-dimethoxybenzyl)amino)thieno[2,3-c]pyridine-2-carboxylate (46 mg, 0.13 mmol) in NH₃/MeOH (5 mL, 7M) and the reaction mixture was stirred at 30° C. for 16 hours. The mixture was concentrated to give 7-((2,4-dimethoxybenzyl)amino)thieno[2,3-c]pyridine-2-carboxamide (43 g, yield 97.7%) as a yellow solid. LC/MS (ESI) m/z: 344 (M+H)⁺.

### Step 6: 2-(aminomethyl)-N-(2,4-dimethoxybenzyl)thieno[2,3-c]pyridin-7-amine (7)

To a solution of 7-((2,4-dimethoxybenzyl)amino)thieno[2,3-c]pyridine-2-carboxamide (50 mg, 0.15 mmol) in THE (3.0 mL) was added LiAIH₄ (1.0 mL, 0.87 mmol, 1.0 M in THF) slowly at 0° C. under N₂ atmosphere and the mixture was stirred at 70° C. for 16 hours. The mixture was quenched with water, aq.15% NaOH, water (1/3/1) and diluted with EtOAc and washed with water, dried over anhydrous Na₂SO₄, filtered and concentrated to dryness to give 2-(aminomethyl)-N-(2,4-dimethoxybenzyl)thieno[2,3-c]pyridin-7-amine (30 mg, yield 62.5%) as a yellow oil. The crude product was used directly in next step. LC/MS (ESI) m/z: 330 (M+H)⁺.

### Step 7: (1S,3S,5S)-N-((7-((2,4-dimethoxybenzyl)amino)thieno[2,3-c]pyridin-2-yl)methyl)-5-methyl-2-((4-phenoxybenzoyl)glycyl)-2-azabicyclo[3.1.0]hexane-3-carboxamide (8)

To a mixture of (1 S,3S,5S)-5-methyl-2-((4-phenoxybenzoyl)glycyl)-2-azabicyclo[3.1.0]hexane-3-carboxylic acid (45 mg, 0.11 mmol) and 2-(aminomethyl)-N-(2,4-dimethoxybenzyl)thieno[2,3-c]pyridin-7-amine (30 mg, 0.11 mmol) in DMF (3 mL) was added DIPEA (0.21 mL, 0.66 mmol) and T₃P (330 mg, 0.33 mmol, 50% wt. in EtOAc) under N₂ atmosphere and the reaction mixture was stirred at room temperature for 2 hours. The mixture was diluted with EtOAc, washed with saturated aq.NaHCO₃ solution and brine, dried over anhydrous Na₂SO₄, filtered and concentrated under reduced pressure to dryness. The residue was purified by prep-TLC (DCM:MeOH=20:1) to give (1S,3S,5S)-N-((7-((2,4-dimethoxybenzyl)amino)thieno[2,3-c]pyridin-2-yl)methyl)-5-methyl-2-((4-phenoxybenzoyl) glycyl)-2-azabicyclo[3.1.0]hexane-3-carboxamide (30 mg, yield 46.8%) as a yellow oil. LC/MS (ESI) m/z: 706 (M+H)⁺.

### Step 8: (1S,3S,5S)-N-((7-aminothieno[2,3-c]pyridin-2-yl)methyl)-5-methyl-2-((4-phenoxy benzoyl)glycyl)-2-azabicyclo[3.1.0]hexane-3-carboxamide (Compound 256)

To a solution of (1 S,3S,5S)-N-((7-((2,4-dimethoxybenzyl)amino)thieno[2,3-c]pyridin-2-yl)methyl)-5-methyl-2-((4-phenoxybenzoyl)glycyl)-2-azabicyclo[3.1.0]hexane-3-carboxamide (30 mg, 0.04 mmol) in DCM (2 mL) was added TFA (1 mL) and the mixture was stirred at room temperature for 1 hour. The reaction mixture was concentrated to dryness under reduced pressure, co-evaporated with DCM and dried under vacuum. The residue was purified by prep-HPLC to give Compound 256 (3.0 mg, yield 12.5%) as a white solid. ¹H NMR (400 MHz, CD₃OD) δ 8.47 (s, 1H), 7.83 (d, J=8.8 Hz, 1H), 7.78 (d, J=8.8 Hz, 1H), 7.70 (dd, J=5.9, 3.9 Hz, 1H), 7.42 (t, J=7.9 Hz, 2H), 7.22 (dd, J=17.0, 9.6 Hz, 2H), 7.02 (ddd, J=21.8, 14.2, 6.8 Hz, 4H), 6.92 (d, J=8.8 Hz, 1H), 4.71 (d, J=5.5 Hz, 1H), 4.68 (s, 1H), 4.58 (s, 1H), 4.44-4.38 (m, 1H), 4.34 (d, J=3.1 Hz, 1H), 2.53-2.42 (m, 1H), 2.22 (dd, J=13.3, 3.2 Hz, 1H), 2.12 (dd, J=13.5, 5.0 Hz, 1H), 1.31 (d, J=1.7 Hz, 3H), 1.18 (s, 1H), 0.84 (dd, J=26.1, 5.6 Hz, 1H). LC/MS (ESI) (m/z): 556 (M+H)⁺.

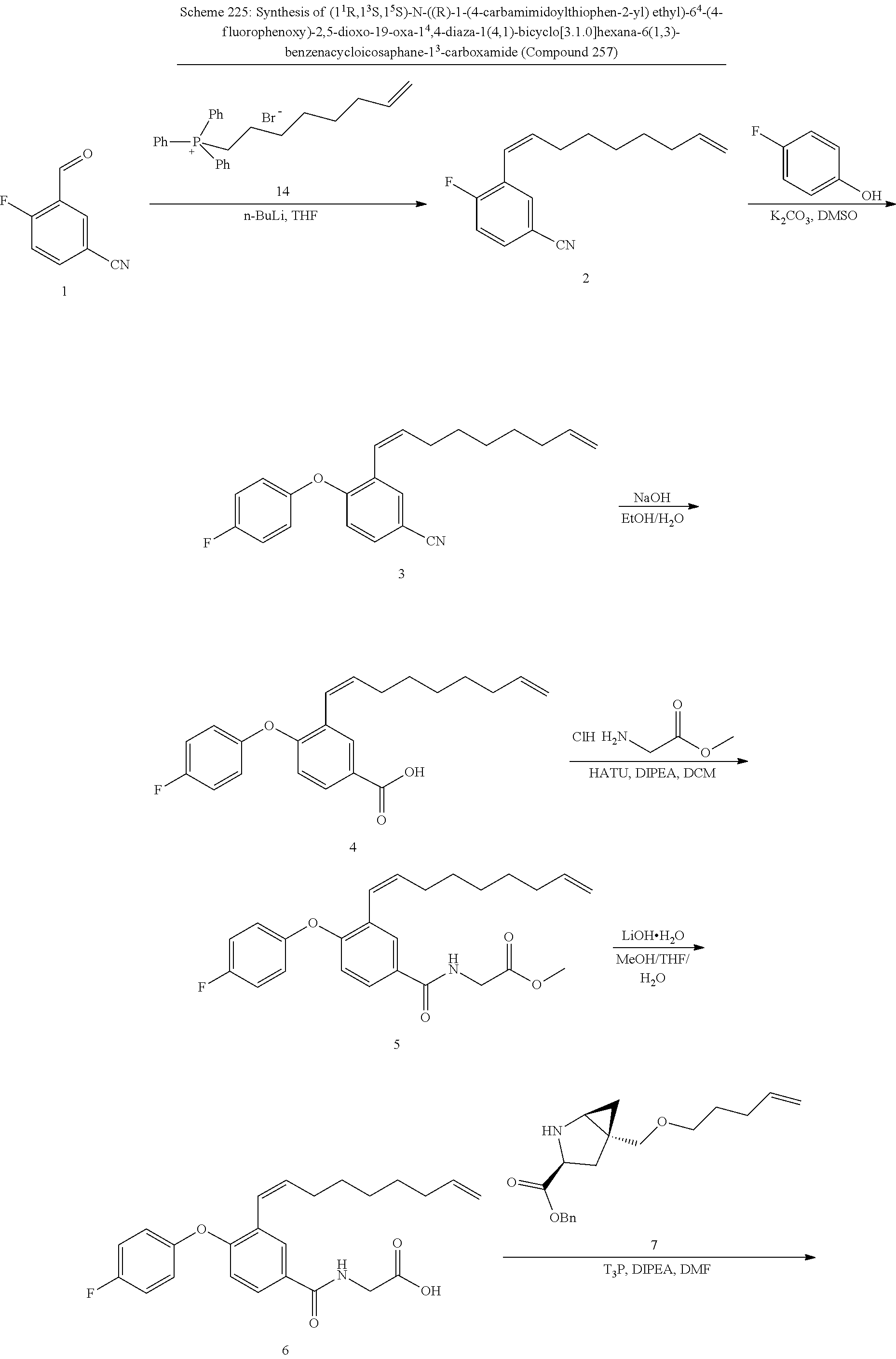

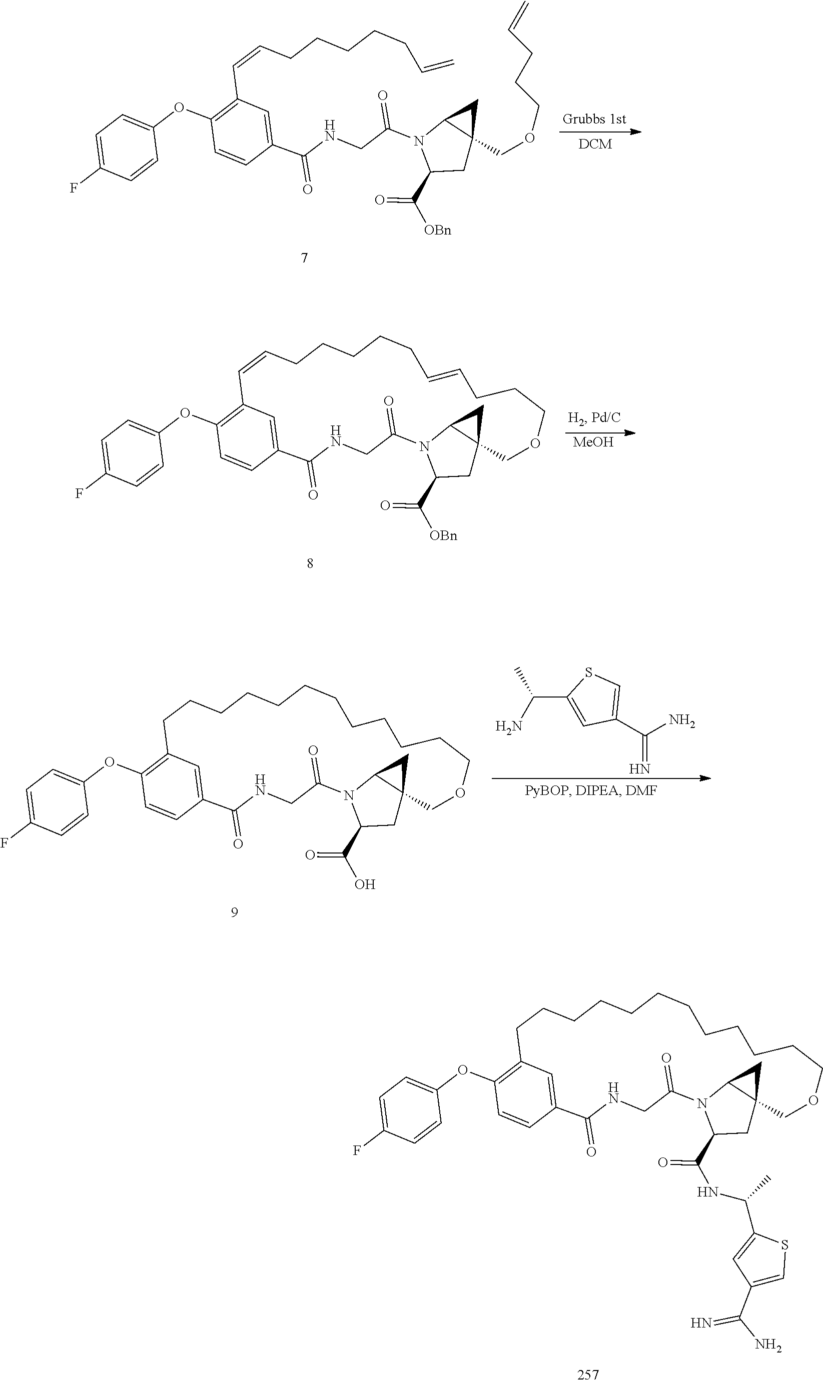

### Step 1: 4-fluoro-3-[(1Z)-nona-1,8-dien-1-yl]benzonitrile

To a solution of oct-7-en-1-yltriphenylphosphanium bromide (10.18 g, 22.53 mmol) in THE (100 mL) was added n-butyllithium (9.76 mL, 24.41 mmol) at −78° C. under N₂ atmosphere and the mixture was stirred at 0° C. for 30 minutes. Then a solution of 4-fluoro-3-formylbenzonitrile (2.8 g, 18.78 mmol) in THE (30 mL) was drop-wise added to the stirring reaction mixture at −78° C. and the mixture was stirred at room temperature for 3 hours under N₂ atmosphere. The mixture was quenched with ice-water and extracted with EtOAc twice. The combined organic layers were washed with brine, dried with anhydrous Na₂SO₄, filtered and concentrated under reduce pressure. The residue was purified by chromatography on silica gel (PE:EtOAc=80:1) to give 4-fluoro-3-[(1Z)-nona-1,8-dien-1-yl]benzonitrile (4.0 g, yield 87.6%) as a light oil. LC/MS(ESI) m/z: 244 (M+H)⁺.

### Step 2: 4-(4-fluorophenoxy)-3-[(1Z)-nona-1,8-dien-1-yl]benzonitrile

To a solution of 4-fluoro-3-[(1Z)-nona-1,8-dien-1-yl]benzonitrile (2.0 g, 8.22 mmol) in DMSO (32 mL) was added K₂CO₃ (2.27 g, 16.44 mmol) and 4-fluorophenol (1.11 g, 9.86 mmol). The reaction mixture was stirred at 120° C. for 16 hours in a seal tube. The mixture was diluted with EtOAc and washed with water. The aqueous layer was extracted with EtOAc and the combined organic layers were washed with brine, dried over anhydrous Na₂SO₄, filtered and concentrated to dryness under reduced pressure. The residue was purified by chromatography on silica gel (PE:EtOAc=85:1) to give 4-(4-fluorophenoxy)-3-[(1Z)-nona-1,8-dien-1-yl]benzonitrile (2.67 g, yield 96.8%) as a light oil. LC/MS(ESI) m/z: 336 (M+H)⁺.

### Step 3: 4-(4-fluorophenoxy)-3-[(1Z)-nona-1,8-dien-1-yl]benzoic acid

To a solution of 4-(4-fluorophenoxy)-3-[(1Z)-nona-1,8-dien-1-yl]benzonitrile (2.67 g, 7.96 mmol) in EtOH (64 mL) and water (56 mL) was added NaOH (6.37 g, 159.20 mmol) at 0° C., and the mixture was the mixture was stirred at 90° C. for 16 hours. The mixture was acidified with 1N aq.HCl to pH=3, extracted with EtOAc twice. The combined organic layers were washed with brine, dried over anhydrous Na₂SO₄, filtered and concentrated under reduced pressure to dryness to give 4-(4-fluorophenoxy)-3-[(1Z)-nona-1,8-dien-1-yl]benzoic acid (2.8 g, yield 99.2%) as a light oil, which was used directly in the next step. LC/MS(ESI) m/z: 355 (M+H)⁺.

### Step 4: methyl 2-{[4-(4-fluorophenoxy)-3-[(1Z)-nona-1,8-dien-1-yl]phenyl]formamido}acetate

To a mixture of 4-(4-fluorophenoxy)-3-[(1Z)-nona-1,8-dien-1-yl]benzoic acid (2.8 g, 7.9 mmol) and methyl 2-aminoacetate hydrochloride (1.19 g, 9.48 mmol) in DCM (30 mL) was added DIPEA (5.11 g, 39.5 mmol) and HATU (4.51 g, 11.85 mmol) under N₂ atmosphere and the reaction mixture was stirred at room temperature for 1 hour. The mixture was diluted with EtOAc and washed with water and brine, dried over anhydrous Na₂SO₄, filtered and concentrated under reduced pressure to dryness. The residue was purified by flash chromatography on silica gel (PE:EtOAc=8:1) to give methyl 2-{[4-(4-fluorophenoxy)-3-[(1Z)-nona-1,8-dien-1-yl]phenyl]formamido}acetate (3.3 g, yield 98.2%) as a yellow oil. LC/MS (ESI) m/z:426 (M+H)⁺.

### Step 5: {[4-(4-fluorophenoxy)-3-[(1Z)-nona-1,8-dien-1-yl]phenyl]formamido}acetic acid

To a solution of methyl 2-{[4-(4-fluorophenoxy)-3-[(1Z)-nona-1,8-dien-1-yl]phenyl]formamido}acetate (3.3 g, 7.76 mmol) in THF/MeOH/H₂O (40 mL, 2/1/1) was added LiOH (1.30 g, 31.02 mmol) and the reaction mixture was stirred at room temperature for 2 hours. The mixture was concentrated to dryness and the residue was diluted with water, washed with EtOAc twice. The aqueous layer was acidified with 0.5 N aq.HCl to pH-4 and extracted with EtOAc twice. The combined organic layers were washed with brine, dried over anhydrous Na₂SOI, filtered and concentrated under reduced pressure to dryness to give {[4-(4-fluorophenoxy)-3-[(1Z)-nona-1,8-dien-1-yl]phenyl]formamido}acetic acid (2.90 g, yield 90.9%) as a light oil. LC/MS (ESI) m/z:412 (M+H)⁺.

### Step 6: benzyl (1S,3S,5R)-2-(2-{[4-(4-fluorophenoxy)-3-[(1Z)-nona-1,8-dien-1-yl]phenyl]formamido}acetyl)-5-[(pent-4-en-1-yloxy)methyl]-2-azabicyclo[3.1.0]hexane-3-carboxylate

To a mixture of benzyl (1S,3S,5R)-5-[(pent-4-en-1-yloxy)methyl]-2-azabicyclo[3.1.0]hexane-3-carboxylate hydrochloride (840 mg, 2.39 mmol) and {[4-(4-fluorophenoxy)-3-[(1Z)-nona-1,8-dien-1-yl]phenyl]formamido}acetic acid (820 mg, 1.99 mmol) in DMF (10 mL) was added DIPEA (1.74 mL, 9.95 mmol) and T₃P (2.53 g, 3.98 mmol, 50% wt. in EtOAc) at 0° C. and the reaction mixture was stirred at room temperature for 1 hour. The mixture was diluted with EtOAc, washed with saturated aq.NaHCO₃ and brine, dried over anhydrous Na₂SO₄, filtered and concentrated under reduced pressure to dryness. The residue was purified by column chromatography on silica gel (PE:EtOAc=3:1) to give benzyl (1S,3S,5R)-2-(2-{[4-(4-fluorophenoxy)-3-[(1Z)-nona-1,8-dien-1-yl]phenyl]formamido}acetyl)-5-[(pent-4-en-1-yloxy)methyl]-2-azabicyclo[3.1.0]hexane-3-carboxylate (950 mg, yield 67.4%) as a yellow oil. LC/MS(ESI) m/z: 709 (M+H)⁺.

### Step 7: benzyl (1R,7E,14Z,26S,29S)-17-(4-fluorophenoxy)-21,24-dioxo-3-oxa-22,25-diazatetracyclo[23.2.2.1¹⁶,²⁰0¹,²⁶]triaconta-7,14,16,18,20(30)-pentaene-29-carboxylate

To a solution of benzyl (1 S,3S,5R)-2-(2-{[4-(4-fluorophenoxy)-3-[(1Z)-nona-1,8-dien-1-yl]phenyl]formamido}acetyl)-5-[(pent-4-en-1-yloxy)methyl]-2-azabicyclo[3.1.0]hexane-3-carboxylate (400 mg, 0.56 mmol) in dry DCM (320 mL) was added Grubbs 1^{st} catalyst (465 mg, 0.56 mmol) and the mixture was stirred at 25° C. for 48 hours under N₂ atmosphere. The mixture was concentrated to dryness under reduced pressure and the residue was purified by flash chromatography (DCM:MeOH=60:1) and prep-HPLC to give benzyl (1R,7E,14Z,26S,29S)-17-(4-fluorophenoxy)-21,24-dioxo-3-oxa-22,25-diazatetracyclo[23.2.2.1¹⁶,²⁰.0¹,²⁶]triaconta-7,14,16,18,20(30)-pentaene-29-carboxylate (30 mg, yield 7.8%) as a brown solid. LC/MS (ESI) m/z: 681 (M+H)⁺.

### Step 8: (1R,26S,29S)-17-(4-fluorophenoxy)-21,24-dioxo-3-oxa-22,25-diazatetracyclo [23.2.2.1¹⁶,²⁰.0¹,²⁶]triaconta-16,18,20(30)-triene-29-carboxylic acid

To a solution of benzyl (1R,19S,25S,28S,E)-17,20,23-trioxo-19-(2-phenoxyethyl)-3-oxa-18,21,24-triazatricyclo[22.2.2.0^{1,25}]octacos-7-ene-28-carboxylate (20 mg, 0.029 mmol) in MeOH (4 mL) was added Pd/C (5 mg, 10% wt.), and the reaction mixture was stirred at room temperature under H₂ atmosphere for 0.5 hours. The mixture was filtered and concentrated to give (1R,26S,29S)-17-(4-fluorophenoxy)-21,24-dioxo-3-oxa-22,25-diazatetracyclo [23.2.2.1¹⁶,²⁶.0¹,²⁶]triaconta-16,18,20(30)-triene-29-carboxylic acid (14 mg, yield 80.1%) as a light oil, which was used directly in the next step without further purification. LC/MS (ESI) m/z: 595(M+H)⁺.

### Step 9: (1R,26S,29S)-N-[(1R)-1-(4-carbamimidoylthiophen-2-yl)ethyl]-17-(4-fluorophenoxy)-21,24-dioxo-3-oxa-22,25-diazatetracyclo[23.2.2.1¹⁶,²⁰.0¹,²⁶]triaconta-16,18,20(30)-triene-29-carboxamide (Compound 257)

To a mixture of (1R,26S,29S)-17-(4-fluorophenoxy)-21,24-dioxo-3-oxa-22,25-diazatetracyclo [23.2.2.1¹⁶,²⁰.0¹,²⁶]triaconta-16,18,20(30)-triene-29-carboxylic acid (12 mg, 0.02 mmol) and 5-[(1R)-1-aminoethyl]thiophene-3-carboximidamide (10 mg, 0.06 mmol) in DMF (2 mL) was added DIPEA (16 mg, 0.0.12 mmol) and T₃P (10 mg, 0.016 mmol) at 0° C. under N₂ atmosphere and the mixture was stirred at 40° C. overnight before 5-[(1R)-1-aminoethyl]thiophene-3-carboximidamide (10 mg, 0.061 mmol) was added to the reaction mixture and stirred at 40° C. for 10 hours. The mixture was diluted with water and extracted with EtOAc twice. The combined organic layers were dried over anhydrous Na₂SO₄, filtered and concentrated under reduced pressure to dryness. The residue was purified by prep-HPLC to give Compound 257 (2.5 mg, yield 20.8%) as a brown solid. ¹H NMR (400 MHz, CD₃OD) δ 8.27-8.21 (m, 1H), 7.82 (s, 1H), 7.64-7.60 (m, 1H), 7.56 (s, 1H), 7.16-7.11 (m, 2H), 7.02-6.97 (m, 2H), 6.80-6.75 (m, 1H), 5.36-5.33 (m, 1H), 5.26-5.23 (m, 1H), 4.27-4.23 (m, 2H), 3.68-3.65 (m, 1H), 3.59-3.52 (m, 2H), 3.50-3.46 (m, 2H), 3.41-3.39 (m, 1H), 2.76-2.72 (m, 2H), 2.66-2.61 (m, 1H), 2.22-2.17 (m, 2H), 2.10-2.01 (m, 5H), 1.71-1.68 (m, 2H), 1.65-1.63 (m, 4H), 1.56-1.53 (m, 2H), 1.35-1.33 (m, 8H), 1.11 (dd, J=5.6, 5.6 Hz, 1H), 0.86-0.83 (m, 1H). LC/MS (ESI) m/z: 746(M+H)⁺.

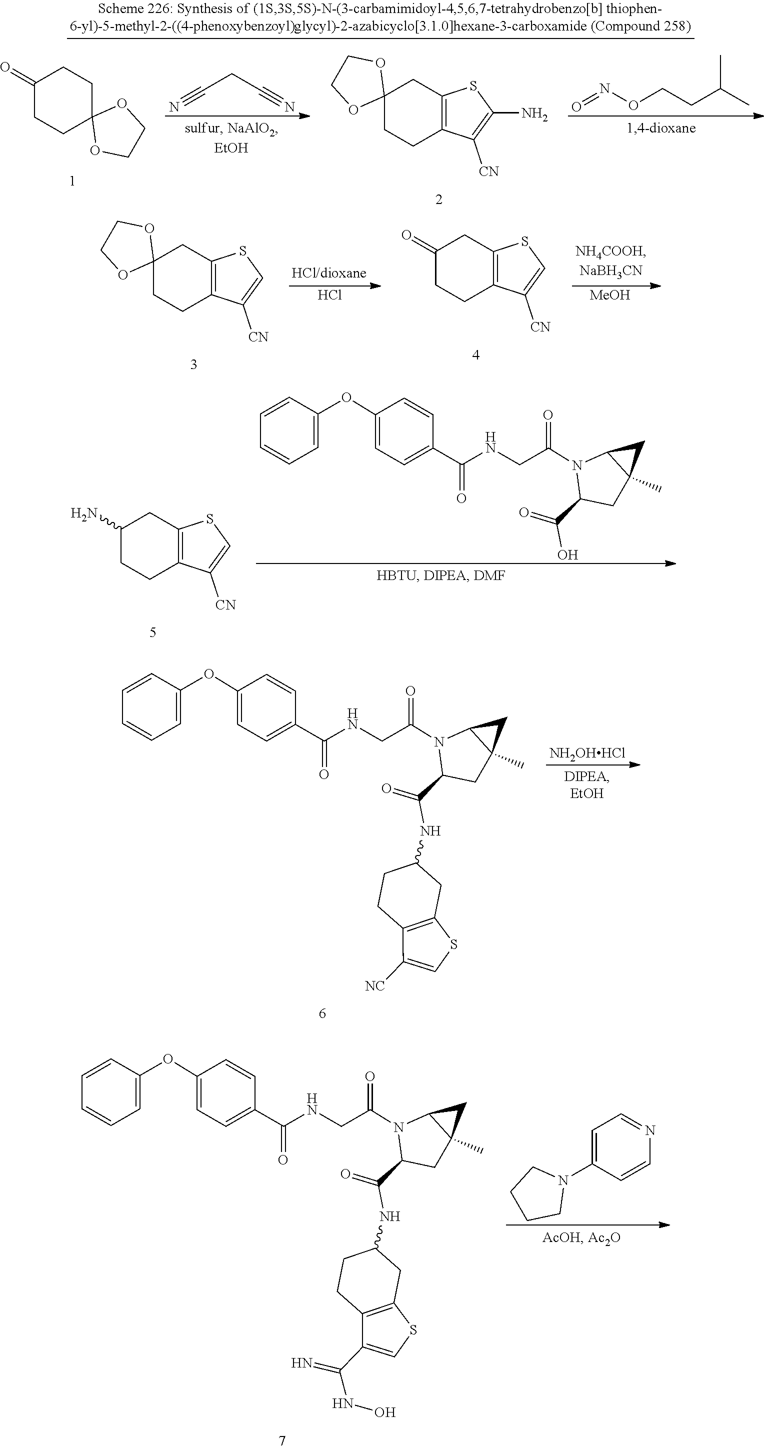

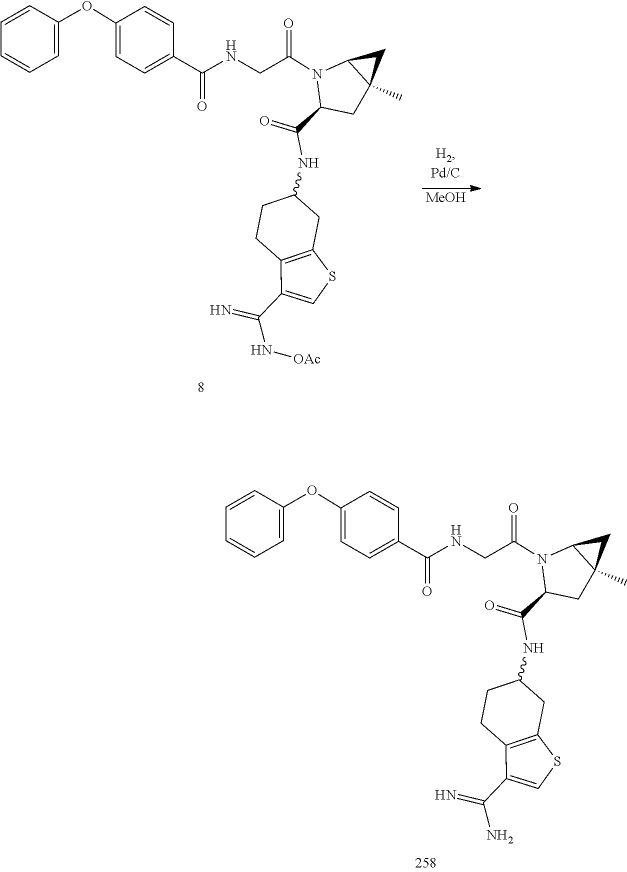 [SMILES: "MeOH" ; CC(=O)ONC(=N)c1csc2c1CCC(NC(=O)[C@@H]1C[C@]3(C)C[C@@H]3N1C(=O)CNC(=O)c1ccc(Oc3ccccc3)cc1)C2 ; C[C@@]12C[C@@H]1N(C(=O)CNC(=O)c1ccc(Oc3ccccc3)cc1)[C@H](C(=O)NC1CCc3c(C(=N)N)csc3C1)C2]

### Step 1: 2-amino-4,7-dihydro-5H-spiro[benzo[b]thiophene-6,2′-[1,3]dioxolane]-3-carbonitrile

To a solution of 1,4-dioxaspiro[4.5]decan-8-one (5 g, 32 mmol) in EtOH (64 mL) was added malononitrile (2.1 g, 32 mmol), sulfur (8.9 g, 35 mmol) and NaAIO₂ (130 mg, 1.6 mmol) under N₂ atmosphere at 60° C. and stirred overnight. The mixture was diluted with water and extracted with EtOAc twice. The combined organic layers were washed with brine, dried over anhydrous Na₂SO₄, filtered and concentrated to dryness under reduced pressure. The residue was purified by column chromatography on silica gel (PE:EtOAc=2:1) to give 2-amino-4,7-dihydro-5H-spiro[benzo[b]thiophene-6,2′-[1,3]dioxolane]-3-carbonitrile (6.2 g, yield 82.2%) as a yellow solid. LCMS (ESI) m/z=237 (M+H)⁺.

### Step 2: 4,7-dihydro-5H-spiro[benzo[b]thiophene-6,2′-[1,3]dioxolane]-3-carbonitrile

To a solution of 2-amino-4,7-dihydro-5H-spiro[benzo[b]thiophene-6,2′-[1,3]dioxolane]-3-carbonitrile (5 g, 32 mmol) in 1,4-dioxane (60 mL) was added isopentyl nitrite (3.3 g, 28 mmol), under N₂ atmosphere at 100° C. and the reaction mixture was stirred overnight. The mixture was diluted with water and extracted with EtOAc twice. The combined organic layers were washed with brine, dried over anhydrous Na₂SO₄, filtered and concentrated to dryness under reduced pressure. The residue was purified by column chromatography on silica gel (PE:EtOAc=3:1) to give 4,7-dihydro-5H-spiro[benzo[b]thiophene-6,2′-[1,3]dioxolane]-3-carbonitrile (2.8 g, yield 49.2%) as a brown solid. LCMS (ESI) m/z=222 (M+H)⁺.

### Step 3: 6-oxo-4,5,6,7-tetrahydrobenzo[b]thiophene-3-carbonitrile

To a solution of 4,7-dihydro-5H-spiro[benzo[b]thiophene-6,2′-[1,3]dioxolane]-3-carbonitrile (2.8 g, 13 mmol) in HCl/1,4-dioxane (2.5 mL) was stirred under N₂ atmosphere at room temperature for 2 hours.

The reaction mixture was concentrated to dryness under reduced pressure, diluted with DCM again and dried under vacuum to give 6-oxo-4,5,6,7-tetrahydrobenzo[b]thiophene-3-carbonitrile (1.5 g, yield 66.9%), which was used directly in the next step without further purification. LC/MS (ESI) m/z: 178 (M+H)⁺.

### Step 4: 6-amino-4,5,6,7-tetrahydrobenzo[b]thiophene-3-carbonitrile

To a solution of 6-oxo-4,5,6,7-tetrahydrobenzo[b]thiophene-3-carbonitrile (1.4 g, 7.9 mmol) in MeOH (15 mL) was added NH₄COOH (2 g, 32 mmol) and NaBH₃CN (347 mg, 5.5 mmol) under N₂ atmosphere at room temperature and stirred overnight. The mixture was diluted with water and extracted with EtOAc twice. The combined organic layers were washed with brine, dried over anhydrous Na₂SO₄, filtered and concentrated to dryness under reduced pressure. The residue was purified by column chromatography on silica gel (DCM:MeOH=15:1) to give 6-amino-4,5,6,7-tetrahydrobenzo[b]thiophene-3-carbonitrile (260 mg, yield 18.5%) as a yellow solid. LCMS (ESI) m/z=179 (M+H)⁺.

### Step 5: (1S,3S,5S)-N-(3-cyano-4,5,6,7-tetrahydrobenzo[b]thiophen-6-yl)-5-methyl-2-((4-phenoxybenzoyl)glycyl)-2-azabicyclo[3.1.0]hexane-3-carboxamide

To a mixture of 6-amino-4,5,6,7-tetrahydrobenzo[b]thiophene-3-carbonitrile (62 mg, 0.35 mmol) and (1S,3S,5S)-5-methyl-2-((4-phenoxybenzoyl)glycyl)-2-azabicyclo[3.1.0]hexane-3-carboxylic acid (125 mg, 0.32 mmol) in DMF (1 mL) was added DIPEA (248 mg, 1.9 mmol) and HBTU (160 mg, 0.42 mmol) at 0° C. under N₂ atmosphere and the mixture was stirred at 25° C. for 1 hour. The mixture was diluted with EtOAc and washed with water twice. The combined organic layers were dried over anhydrous Na₂SO₄, filtered and concentrated to dryness under reduced pressure. The residue was purified by column chromatography on silica gel (DCM:MeOH=12:1) to give (1S,3S,5S)-N-(3-cyano-4,5,6,7-tetrahydrobenzo[b]thiophen-6-yl)-5-methyl-2-((4-phenoxybenzoyl)glycyl)-2-azabicyclo[3.1.0]hexane-3-carboxamide (120 mg, 61.9%). LC/MS (ESI) m/z: 555 (M+H)⁺.

### Step 6: (1S,3S,5S)-N-(3-(N-hydroxycarbamimidoyl)-4,5,6,7-tetrahydrobenzo[b]thiophen-6-yl)-5-methyl-2-((4-phenoxybenzoyl)glycyl)-2-azabicyclo[3.1.0]hexane-3-carboxamide

To a solution of (1S,3S,5S)-N-(3-cyano-4,5,6,7-tetrahydrobenzo[b]thiophen-6-yl)-5-methyl-2-((4-phenoxybenzoyl)glycyl)-2-azabicyclo[3.1.0]hexane-3-carboxamide (120 mg, 0.22 mmol) in EtOH (1.5 mL) was added NH₂OH·HCl (38 mg, 0.54 mmol) and DIPEA (168 mg, 1.3 mmol) under N₂ atmosphere at room temperature and the reaction mixture stirred overnight. The mixture was diluted with water and extracted with EtOAc twice. The combined organic layers were washed with brine, dried over anhydrous Na₂SO₄, filtered and concentrated to dryness under reduced pressure. The residue was purified by column chromatography on silica gel (DCM:MeOH=10:1) to give (1S,3S,5S)-N-(3-(N-hydroxycarbamimidoyl)-4,5,6,7-tetrahydrobenzo[b]thiophen-6-yl)-5-methyl-2-((4-phenoxybenzoyl)glycyl)-2-azabicyclo[3.1.0]hexane-3-carboxamide (76 mg, yield 58.8%) as a yellow oil. LCMS (ESI) m/z=588 (M+H)⁺.

### Step 7: (1S,3S,5S)-N-(3-(N-acetoxycarbamimidoyl)-4,5,6,7-tetrahydrobenzo[b]thiophen-6-yl)-5-methyl-2-((4-phenoxybenzoyl)glycyl)-2-azabicyclo[3.1.0]hexane-3-carboxamide

To a mixture of (1 S,3S,5S)-N-(3-(N-hydroxycarbamimidoyl)-4,5,6,7-tetrahydrobenzo [b]thiophen-6-yl)-5-methyl-2-((4-phenoxybenzoyl)glycyl)-2-azabicyclo[3.1.0]hexane-3-carboxamide (76 mg, 0.13 mmol) in AcOH (0.5 mL) and AC20 (0.5 mL) was added 4-(pyrrolidin-1-yl)pyridine (1.9 mg, 0.013 mmol) under N₂ atmosphere at room temperature and the reaction mixture was stirred for 2 hours. The mixture was diluted with water and extracted with EtOAc twice. The combined organic layers were washed with brine, dried over anhydrous Na₂SO₄, filtered and concentrated to dryness under reduced pressure. The residue was purified by column chromatography on silica gel (DCM:MeOH=12:1) to give (1 S,3S,5S)-N-(3-(N-acetoxycarbamimidoyl)-4,5,6,7-tetrahydrobenzo[b]thiophen-6-yl)-5-methyl-2-((4-phenoxybenzoyl)glycyl)-2-azabicyclo[3.1.0]hexane-3-carboxamide (60 mg, yield 73.4%) as a yellow solid. LCMS (ESI) m/z=630 (M+H)⁺.

### Step 8: (1S,3S,5S)-N-(3-carbamimidoyl-4,5,6,7-tetrahydrobenzo[b]thiophen-6-yl)-5-methyl-2-((4-phenoxybenzoyl)glycyl)-2-azabicyclo[3.1.0]hexane-3-carboxamide (Compound 258)

To a solution of (1 S,3S,5S)-N-(3-(N-acetoxycarbamimidoyl)-4,5,6,7-tetrahydrobenzo[b]thiophen-6-yl)-5-methyl-2-((4-phenoxybenzoyl)glycyl)-2-azabicyclo[3.1.0]hexane-3-carboxamide (60 mg, 0.095 mmol) in MeOH (2 mL) was added Pd/C (20 mg, 10% wt.) at room temperature and the mixture was stirred under H₂ atmosphere at room temperature for 2 hours. The mixture was filtered and the filtrate was concentrated to dryness under reduced pressure and purified by prep-HPLC to give Compound 258 (35 mg, yield 64.5%) as a white solid. ¹H NMR (400 MHz, CD₃OD) δ 8.54 (s, 1H), 7.91-7.84 (m, 3H), 7.41 (t, J=7.9 Hz, 2H), 7.20 (t, J=7.4 Hz, 1H), 7.06 (d, J=8.1 Hz, 2H), 7.00 (d, J=8.7 Hz, 2H), 4.80-4.76 (m, 1H), 4.58 (s, 2H), 4.38-4.25 (m, 2H), 3.16-3.07 (m, 1H), 2.80 (d, J=14.8 Hz, 3H), 2.39 (t, J=12.4 Hz, 1H), 2.13 (ddd, J=56.7, 30.9, 6.7 Hz, 3H), 1.30 (s, 3H), 1.19 (dd, J=24.1, 3.3 Hz, 1H).LC/MS (ESI) (m/z): 572 (M+H)⁺.

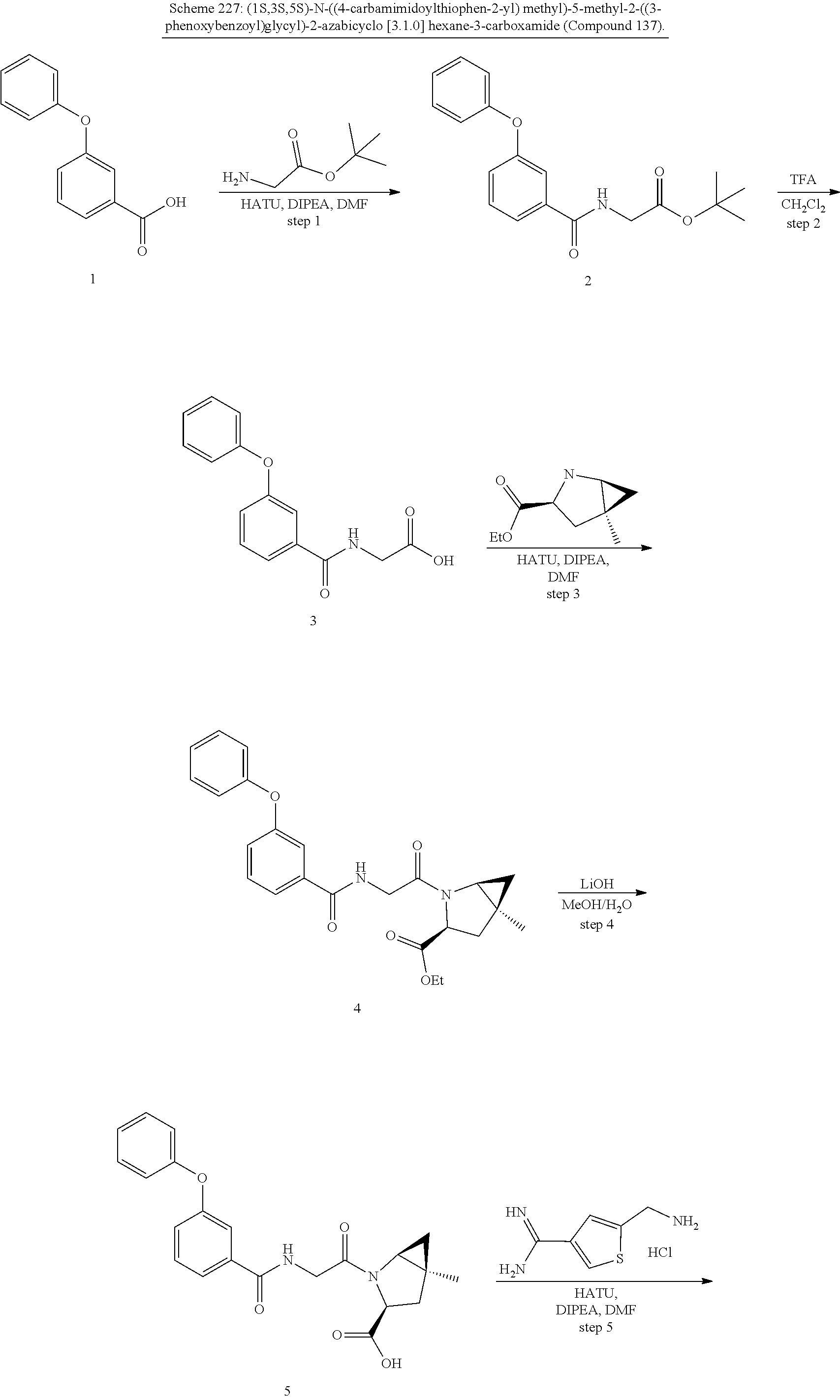

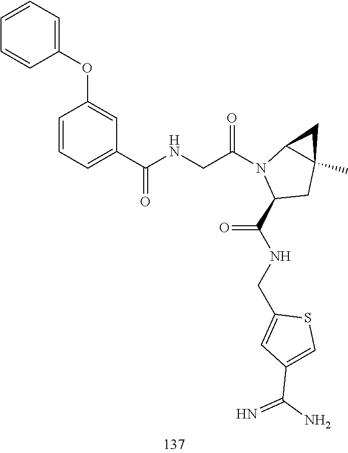 [SMILES: C[C@@]12C[C@@H]1N(C(=O)CNC(=O)c1cccc(Oc3ccccc3)c1)[C@H](C(=O)NCc1cc(C(=N)N)cs1)C2]

### Step 1: tert-butyl 2-[(3-phenoxyphenyl)formamido]acetate (2)

To a solution of 3-phenoxybenzoic acid, 1 (1 g, 4.67 mmol, 1 equiv.) in DMF (5 mL) was added tert-butyl 2-aminoacetate (0.67 g, 5.14 mmol, 1.1 equiv.), HATU (2.31 g, 6.07 mmol, 1.3 equiv.) and DIPEA (3.26 mL, 18.67 mmol, 4 equiv.). The reaction was stirred at room temperature for 1 h. Water (10 mL) was added and the solid collected by filtration and then dried to give compound 2 (1.2 g, 3.67 mmol, Yield 78.59%) as a white solid. LC/MS (ESI) m/z: 328 (M+H)⁺.

### Step 2: (3-phenoxybenzoyl)glycine (3)

TFA (2.34 mL) was added to a solution of compound 2 (1 g, 3.05 mmol, 1 equiv.) in CH₂Cl₂ (5 mL) at ice-bath temperature. The reaction was stirred at room temperature for 2 hours and then concentrated to dryness to give compound 3 (0.8 g, 2.95 mmol, Yield 96.72%) as a white solid. LC/MS (ESI) m/z: 272 (M+H)⁺.

### Step 3: ethyl (1S,3S,5S)-5-methyl-2-((3-phenoxybenzoyl)glycyl)-2-azabicyclo[3.1.0]hexane-3-carboxylate (4)

To a solution of compound 3 (0.1 g, 0.37 mmol, 1 equiv.) in DMF (2 mL) was added ethyl (1S,3S,5S)-5-methyl-2-azabicyclo[3.1.0]hexane-3-carboxylate (0.062 g, 0.37 mmol, 1 equiv.), HATU (0.18 g, 0.48 mmol, 1.3 equiv.) and DIPEA (0.16 mL, 0.92 mmol, 2.5 equiv.). The reaction was stirred at room temperature for 30 minutes. Water (5 mL) and CH₂Cl₂ (5 mL) were added. The two layers were separated, and the aqueous layer was extracted with CH₂Cl₂ (5 mL×2). The combined organic layers were washed with brine, dried over Na₂SO₄, and concentrated. Purification by Combi Flash; 4 g column, solvent A=CH₂Cl₂, solvent B=MeOH, 100% A to 5% B gave compound 4 (0.12 g, 0.28 mmol, Yield 77.05%) as a yellow oil. LC/MS (ESI) m/z: 423 (M+H)⁺.

### Step 4: (1S,3S,5S)-5-methyl-2-{2-[(3-phenoxyphenyl)formamido]acetyl}-2-azabicyclo[3.1.0]hexane-3-carboxylic acid (5)

To a solution of compound 4 (0.12 g, 0.28 mmol, 1 equiv.) in MeOH (3 mL) and H₂O (1 mL) was added LiOH (0.014 g, 0.57 mmol, 2 equiv.). The reaction was stirred at room temperature overnight, concentrated to remove MeOH, and then diluted with CH₂Cl₂ (3 mL). The pH was adjusted to 1 using 1 N HCl. The two layers were separated, and the aqueous layer was extracted with CH₂Cl₂ (3 mL×3). The combined organic extracts were washed with brine, dried over Na₂SO₄ and concentrated to give compound 5 (0.1 g, 0.25 mmol, Yield 89.29%) as a white solid. LC/MS (ESI) m/z: 395 (M+H)⁺.

### Step 5: (1S,3S,5S)-N-((4-carbamimidoylthiophen-2-yl)methyl)-5-methyl-2-((3-phenoxbenz oyl)glycyl)-2-azabicyclo[3.1.0]hexane-3-carboxamide (Compound 137)

To a solution of compound 5 (35 mg, 0.09 mmol, 1.0 equiv.) in DMF (2 mL) was added 5-(aminomethyl)thiophene-3-carboximidamide (44 mg, 0.29 mmol, 1.1 equiv.), HATU (37 mg, 0.01 mmol, 1.1 equiv.), and DIPEA (62 μL, 0.36 mmol, 4 equiv.). The reaction was stirred at room temperature for 20 minutes and then purified directly by HPLC to give Compound 137 (13 mg, 0.024 mmol, Yield 27.56%). LC/MS (ESI) m/z: 532 (M+H)⁺. ¹H NMR (400 MHz, DMSO-d₆) δ 9.29 (s, 1H), 8.75 (q, J=7.9, 6.9 Hz, 1H), 8.54 (t, J=5.8 Hz, 1H), 8.42 (s, 1H), 8.33 (d, J=9.5 Hz, 1H), 7.65 (d, J=7.7 Hz, 1H), 7.56-7.37 (m, 6H), 7.24-7.13 (m, 2H), 7.04 (d, J=8.0 Hz, 2H), 4.63 (td, J=13.2, 12.3, 4.4 Hz, 1H), 4.49-4.29 (m, 2H), 4.02 (dd, J=16.4, 5.9 Hz, 1H), 2.28 (t, J=12.3 Hz, 1H), 1.98 (dd, J=13.2, 3.0 Hz, 1H), 1.21 (s, 3H), 0.70-0.58 (m, 2H).

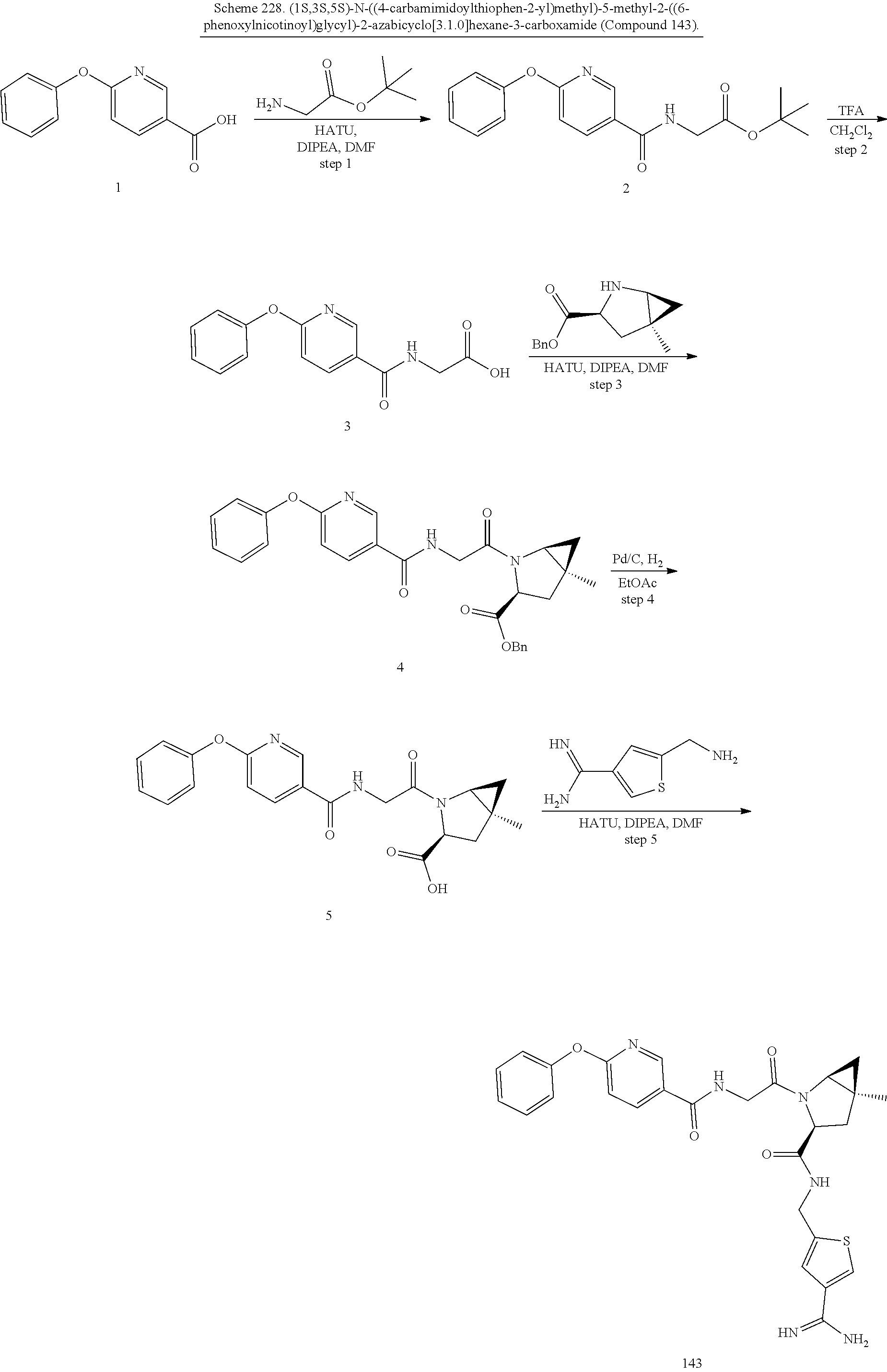

### Step 1: tert-butyl (6-phenoxynicotinoyl)glycinate (2)

To a solution of 4-(pyridin-2-yloxy)benzoic acid, 1 (0.5 g, 2.32 mmol, 1.0 equiv.) in DMF (2 mL) was added, tert-butyl 2-aminoacetate (0.46 g, 3.49 mmol, 1.5 equiv.), HATU (1.325 g, 3.49 mmol, 1.5 equiv.), and finally DIPEA (1.62 mL, 9.29 mmol, 4 equiv.). The reaction was stirred for 30 minutes at room temperature. Water (10 mL) was added. The product crushed out as a sticky oil and was dissolved in CH₂Cl₂, washed with brine, dried over Na₂SO₄, and concentrated to obtain compound 2 (0.65 g, 1.98 mmol, Yield 85.20%). LC/MS (ESI) m/z: 329 (M+H)⁺.

### Step 2: (6-phenoxynicotinoyl)glycine (3)

TFA (1.0 mL) was added to a solution of compound 2 (0.6 g, 1.83 mmol, 1.0 equiv.) in CH₂Cl₂ (3 mL) at ice-bath temperature. The reaction was stirred at room temperature for 3 hours and then concentrated to dryness to give compound 3 (0.45 g, 1.65 mmol, Yield 90.45%) as an off yellow solid which was used in the next step without further purification. LC/MS (ESI) m/z: 273 (M+H)⁺.

### Step 3: benzyl (1S,3S,5S)-5-methyl-2-((6-phenoxynicotinoyl)glycyl)-2-azabicyclo [3.1.0]hexane-3-carboxylate (4)

To a solution of compound 3 (0.1 g, 0.37 mmol, 1.1 equiv.) in DMF (2 mL) was added benzyl (1S,3S,5S)-5-methyl-2-azabicyclo [3.1.0]hexane-3-carboxylate (0.09 g, 0.40 mmol, 1.0 equiv.), HATU (0.18 g, 0.48 mmol, 1.3 equiv.) and DIPEA (0.16 mL, 0.92 mmol, 2.5 equiv.). The reaction was stirred at room temperature for 30 minutes. Water (4 mL) and EtOAc (5 mL) were added. The two layers were separated, and the aqueous layer was extracted with EtOAc (5 mL×2), washed with brine, dried over Na₂SO₄, and concentrated. Purification by Combi Flash; 4 g column, solvent A=CH₂Cl₂, solvent B=MeOH, 100% A to 5% B gave compound 4 (0.13 g, 0.27 mmol, Yield 72.95%). LC/MS (ESI) m/z: 486 (M+H)⁺.

### Step 4: (1S,3S,5S)-5-methyl-2-((6-phenoxynicotinoyl)glycyl)-2-azabicyclo[3.1.0]hexane-3-carboxylic acid (5)

To a solution of compound 4 (50 mg, 0.103 mmol, 1 equiv.) in EtOAc (2 mL) was added 10% Pd/C (11 mg, 0.0103 mmol, 0.1 equiv.). The flask was evacuated and then backfilled with H₂ in a balloon. The reaction was stirred at room temperature overnight, and then concentrated to give compound 5 (35 mg, 0.09 mmol, Yield 85.95%) as a yellow solid. LC/MS (ESI) m/z: 396 (M+H)⁺.

### Step 5: (1S,3S,5S)-N-((4-carbamimidoylthiophen-2-yl)methyl)-5-methyl-2-((6-phenoxynicotinoyl)glycyl)-2-azabicyclo[3.1.0]hexane-3-carboxamide (Compound 143)

To a solution of compound 5 (35 mg, 0.09 mmol, 1.0 equiv.) in DMF (2 mL) was added 5-(aminomethyl)thiophene-3-carboximidamide·HCl (19 mg, 0.1 mmol, 1.1 equiv.), HATU (37 mg, 0.1 mmol, 1.1 equiv.), and DIPEA (62 μL, 0.35 mmol, 4.0 equiv.). The reaction mixture was stirred at room temperature for 20 minutes and then purified directly by HPLC to give Compound 143 (0.010 g, 0.019 mmol, Yield 21.11%). LC/MS (ESI) m/z: 533 (M+H)⁺. ¹H NMR (400 MHz, DMSO-d₆) δ 8.96 (s, 1H), 8.86 (t, J=5.7 Hz, 1H), 8.80 (s, 1H), 8.63 (d, J=2.6 Hz, 1H), 8.56 (t, J=5.9 Hz, 2H), 8.42 (s, 1H), 8.33 (d, J=10.2 Hz, 2H), 8.26 (dd, J=8.6, 2.5 Hz, 2H), 7.56-7.31 (m, 2H), 7.25 (t, J=7.4 Hz, 2H), 7.10 (d, J=8.6 Hz, 2H), 4.66 (dd, J=11.4, 3.1 Hz, 2H), 4.52-4.23 (m, 2H), 4.05 (dd, J=16.6, 5.3 Hz, 3H), 3.62-3.50 (m, 1H), 3.48-3.32 (m, 2H), 2.29 (t, J=12.5 Hz, 1H), 1.99 (dd, J=13.1, 3.1 Hz, 1H), 1.22 (s, 3H), 1.07-0.85 (m, 1H), 0.77-0.55 (m, 1H).

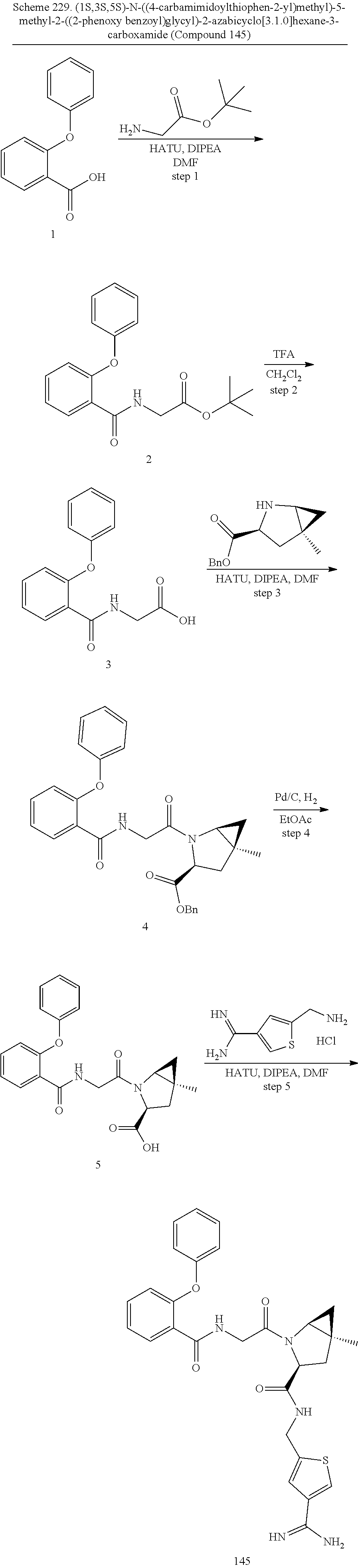

### Step 1: tert-butyl (2-phenoxybenzoyl)glycinate (2)

To a solution of 2-phenoxybenzoic acid, 1 (1.2 g, 5.60 mmol, 1 equiv.) in DMF (5 mL) was added tert-butyl 2-aminoacetate (1.13 g, 6.72 mmol, 1.1 equiv.), HATU (2.77 g, 7.28 mmol, 1.3 equiv.) and DIPEA (2.94 mL, 16.81 mmol, 3 equiv.). The reaction was stirred at room temperature for 1 hour. Water (10 mL) was added and the solid collected by filtration and then dried to give compound 2 (1.5 g, 4.58 mmol, Yield 81.79%) as a white solid. LC/MS (ESI) m/z: 328 (M+H)⁺.

### Step 2: (2-phenoxybenzoyl)glycine (3)

TFA (2.34 mL) was added to a solution of compound 2 (1 g, 3.06 mmol, 1 equiv.) in CH₂Cl₂ (5 mL) at ice-bath temperature. The reaction was stirred at room temperature for 2 hours and then concentrated to dryness to give compound 3 (0.8 g, 2.95 mmol, Yield 96.55%) as white solid. LC/MS (ESI) m/z: 272 (M+H)⁺.

### Step 3: benzyl (1S,3S,5S)-5-methyl-2-((2-phenoxybenzoyl)glycyl)-2-azabicyclo[3.1.0]hexane-3-carboxylate (4)

To a solution of compound 3 (0.21 g, 0.56 mmol, 1.1 equiv.) in DMF (1 mL) was added benzyl (1S,3S,5S)-5-methyl-2-azabicyclo[3.1.0]hexane-3-carboxylate (0.1 g, 0.43 mmol, 1 equiv.), HATU (0.21 g, 0.56 mmol, 1.3 equiv.) and DIPEA (0.23 mL, 1.3 mmol, 2.5 equiv.). The reaction was stirred at room temperature for 30 minutes. Water (5 mL) was added and the liquid was decanted. EtOAc (5 mL) was added to the residue and washed with brine, dried over Na₂SO₄, and concentrated to give compound 4 (0.19 g, 0.39 mmol, Yield 90.69%) as a yellow oil, which was used in the next reaction. LC/MS (ESI) m/z: 485 (M+H)⁺.

### Step 4: (1S,3S,5S)-5-methyl-2-((2-phenoxybenzoyl)glycyl)-2-azabicyclo[3.1.0]hexane-3-carboxylic acid (5)

To a solution of compound 4 (0.19 g, 0.39 mmol, 1 equiv.) in EtOAc (3 mL) was added 10% Pd/C (0.042 g, 0.039 mmol, 0.1 equiv.). The flask was evacuated and then backfilled with H₂ in a balloon.

The reaction was stirred at room temperature overnight and then concentrated to give compound 5 (0.15 g, 0.38 mmol, Yield 97.44%) as a yellow solid. LC/MS (ESI) m/z: 395 (M+H)⁺.

### Step 5: (1S,3S,5S)-N-((4-carbamimidoylthiophen-2-yl)methyl)-5-methyl-2-((2-phenoxybenz oyl)glycyl)-2-azabicyclo[3.1.0]hexane-3-carboxamide (Compound 145)

To a solution of compound 5 (0.15 g, 0.38 mmol, 1.0 equiv.) in DMF (2 mL) was added 5-(aminomethyl)thiophene-3-carboximidamide (0.08 g, 0.42 mmol, 1.1 equiv.), HATU (0.16 g, 0.42 mmol, 1.1 equiv.), and DIPEA (0.27 mL, 1.52 mmol, 4 equiv.). The reaction mixture was stirred at room temperature for 20 minutes and then purified directly by HPLC to give Compound 145 (10 mg, 0.019 mmol, Yield 4.95%). LC/MS (ESI) m/z: 532 (M+H)⁺. ¹H NMR (400 MHz, DMSO-d₆) δ 9.02 (s, 1H), 8.63-8.47 (m, 1H), 8.34 (d, J=1.9 Hz, 2H), 7.91 (t, J=7.0 Hz, 2H), 7.63-7.37 (m, 2H), 7.22 (q, J=7.5 Hz, 1H), 7.09 (d, J=8.0 Hz, 1H), 6.87 (dd, J=8.4, 4.3 Hz, 1H), 4.65 (dd, J=11.6, 2.9 Hz, 2H), 4.53-4.27 (m, 3H), 4.13 (dd, J=17.2, 5.0 Hz, 1H), 3.51-3.30 (m, 2H), 2.39-2.22 (m, 1H), 1.98 (d, J=13.9 Hz, 1H), 1.20 (s, 3H), 0.80-0.7 (m, 1H) 0.67 (t, J=5.7 Hz, 1H).

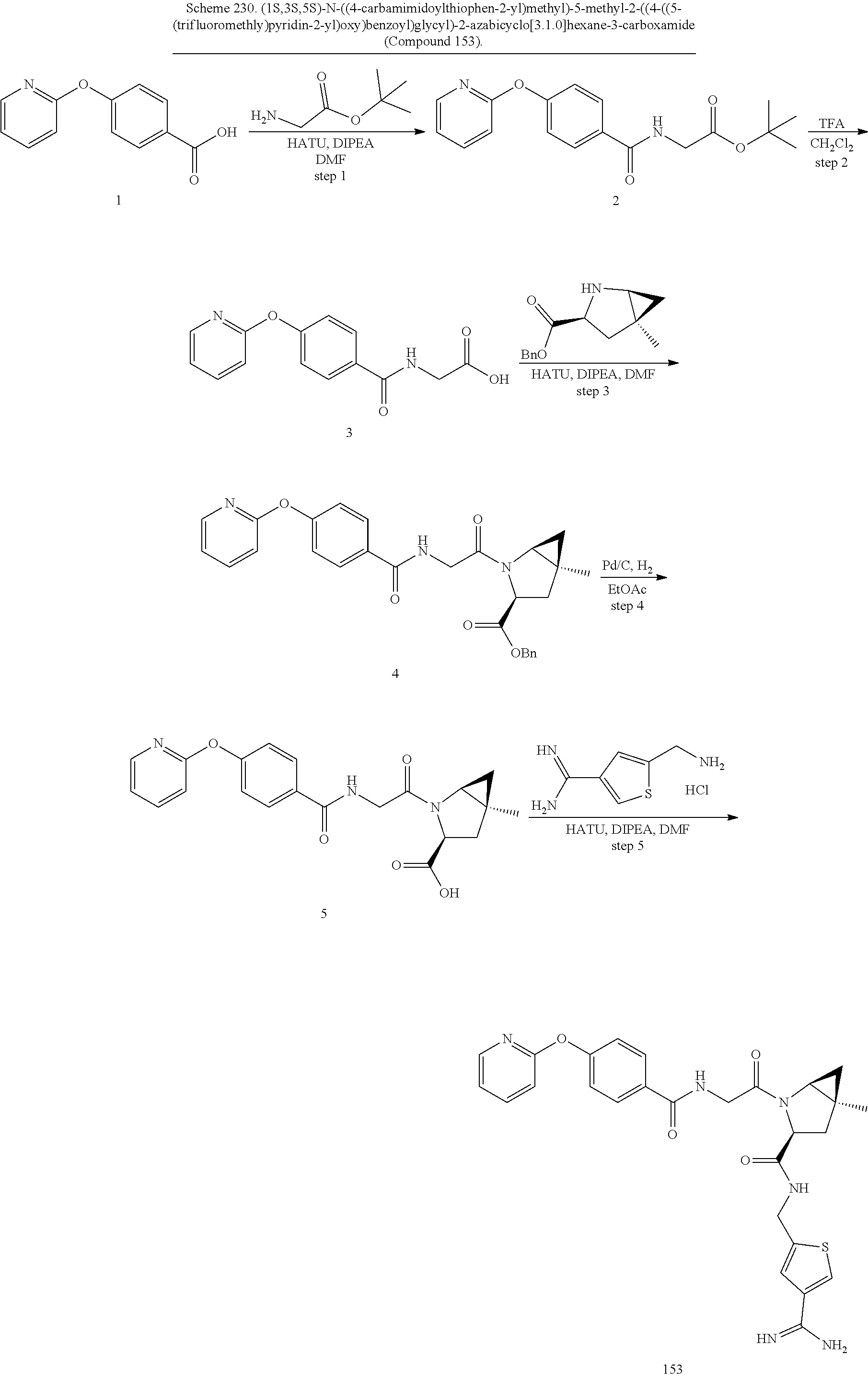

### Step 1: tert-butyl (4-(pyridin-2-yloxy)benzoyl)glycinate (2)

To a solution of 4-(pyridin-2-yloxy)benzoic acid, 1 (0.85 g, 3.95 mmol, 1.0 equiv.) in DMF (2 mL) was added, tert-butyl 2-aminoacetate (0.79 g, 4.74 mmol, 1.2 equiv.), HATU (0.57 g, 1.51 mmol, 1.3 equiv.), and finally DIPEA (2.76 mL, 15.78 mmol, 3 equiv.). The reaction was stirred for 20 minutes at room temperature. Water (5 mL) and EtOAc (5 mL) were added, the two layers were separated, and the aqueous layer was extracted with EtOAc (5 mL×2), washed with brine, dried over Na₂SO₄, and concentrated. Purification by Combi Flash; 24 g column, solvent A=CH₂Cl₂, solvent B=MeOH, 100% A to 5% B gave compound 2 (1.05 g, 3.2 mmol, Yield 80.96%) as a pale yellow solid. LC/MS (ESI) m/z: 329 (M+H)⁺.

### Step 2: (4-(pyridin-2-yloxy)benzoyl)glycine (3)

TFA (2.33 mL) was added to a solution of compound 2 (1.0 g, 3.05 mmol, 1.0 equiv.) in CH₂Cl₂ (5 mL) at ice-bath temperature. The reaction was stirred at room temperature for 2 hours and then concentrated to dryness to give compound 3 (0.8 g, 2.94 mmol, Yield 96.39%) as a white solid. LC/MS (ESI) m/z: 273 (M+H)⁺.

### Step 3: benzyl (1S,3S,5S)-5-methyl-2-((4-(pyridin-2-yloxy)benzoyl)glycyl)-2-azabicyclo [3.1.0]hexane-3-carboxylate (4)

To a solution of compound 3 (0.13 g, 0.48 mmol, 1.2 equiv.) in DMF (2 mL) was added benzyl (1S,3S,5S)-5-methyl-2-azabicyclo[3.1.0]hexane-3-carboxylate (0.2 g, 0.43 mmol, 1.0 equiv.), HATU (0.21 g, 0.56 mmol, 1.3 equiv.) and DIPEA (0.23 mL, 1.3 mmol, 3.0 equiv.). The reaction was stirred at room temperature for 30 minutes. Water (4 mL) and EtOAc (5 mL) were added. The two layers were separated, and the aqueous layer was extracted with EtOAc, washed with brine, dried over Na₂SO₄, and concentrated. Purification by Combi Flash; 4 g column, solvent A=CH₂Cl₂, solvent B=MeOH, 100% A to 5% B gave compound 4 (0.12 g, 0.25 mmol, Yield 57.16%). LC/MS (ESI) m/z: 486 (M+H)⁺.

### Step 4: (1S,3S,5S)-5-methyl-2-((4-(pyridin-2-yloxy)benzoyl)glycyl)-2-azabicyclo [3.1.0]hexane-3-carboxylic acid (5)

To a solution of compound 4 (120 mg, 0.25 mmol, 1 equiv.) in EtOAc (3 mL) was added 10% Pd/C (26 mg, 0.025 mmol, 0.1 equiv.). The flask was evacuated and then backfilled with H₂ in a balloon. The reaction was stirred at room temperature overnight, filtered through Celite pad and then concentrated to give compound 5 (85 mg, 0.21 mmol, Yield 84.0%) as a yellow solid. LC/MS (ESI) m/z: 396 (M+H)⁺.

### Step 5: (1S,3S,5S)-N-((4-carbamimidoylthiophen-2-yl)methyl)-5-methyl-2-((4-(pyridin-2-yloxy)benzoyl)glycyl)-2-azabicyclo[3.1.0]hexane-3-carboxamide (Compound 153)

To a solution of compound 5 (36 mg, 0.09 mmol, 1.0 equiv.) in DMF (2 mL) was added 5-(aminomethyl)thiophene-3-carboximidamide·HCl (16 mg, 0.1 mmol, 1.1 equiv.), HATU (38 mg, 0.1 mmol, 1.1 equiv.), and DIPEA (64 μL, 0.36 mmol, 4.0 equiv.). The reaction mixture was stirred at room temperature for 20 minutes and then purified directly by HPLC to give Compound 153 (15 mg, 0.028 mmol, Yield 31.11%). LC/MS (ESI) m/z: 533 (M+H)⁺. ¹H NMR (400 MHz, DMSO-d₆)¹H NMR (400 MHz, DMSO) δ 8.96 (s, 1H), 8.70 (t, J=5.7 Hz, 2H), 8.66 (s, 1H), 8.55 (t, J=5.9 Hz, 1H), 8.44-8.40 (s, 1H), 8.33 (d, J=10.4 Hz, 1H), 8.18 (d, J=5.0 Hz, 1H), 7.97-7.78 (m, 2H), 7.46 (s, 2H), 7.19 (t, J=8.0 Hz, 2H), 7.10 (d, J=8.2 Hz, 2H), 4.67 (dd, J=11.3, 3.1 Hz, 2H), 4.53-4.16 (m, 2H), 4.06 (dd, J=16.5, 5.8 Hz, 2H), 3.71-3.47 (m, 1H), 2.29 (t, J=12.5 Hz, 2H), 2.00 (dd, J=13.5, 3.1 Hz, 1H), 1.22 (d, J=11.2 Hz, 3H), 0.81-0.60 (m, 2H).

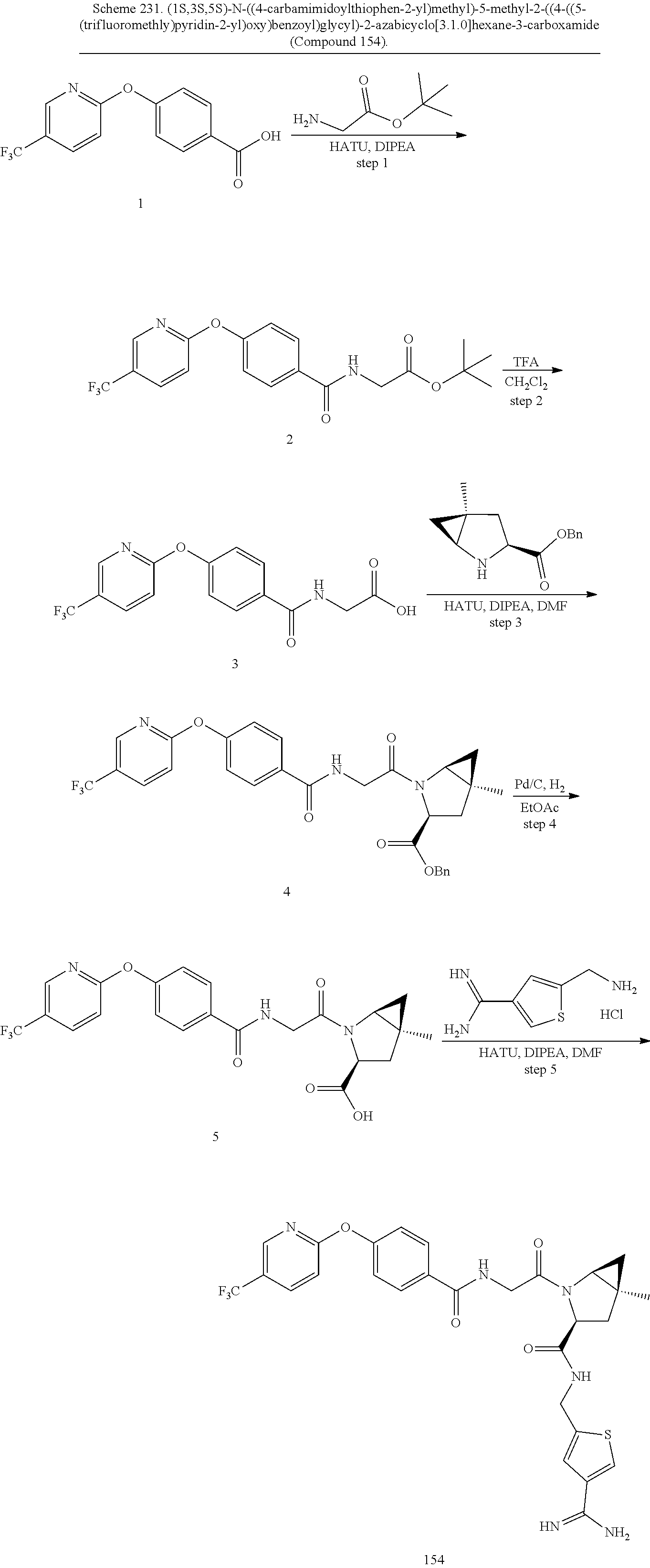

### Step 1: tert-butyl (4-((5-(trifluoromethyl)pyridin-2-yl)oxy)benzoyl)glycinate(2)

To a solution of 4-((5-(trifluoromethyl)pyridin-2-yl)oxy)benzoic acid, 1 (1 g, 3.53 mmol, 1 equiv.) in DMF (5 mL) was added tert-butyl 2-aminoacetate (0.59 g, 3.53 mmol, 1.1 equiv.), HATU (1.75 g, 4.59 mmol, 1.3 equiv.) and DIPEA (1.85 mL, 10.59 mmol, 3 equiv.). The reaction was stirred at room temperature for 1 h. Water (10 mL) was added and the solid was collected by filtration and then dried to give compound 2 (1.2 g, 3.03 mmol, Yield 85.84%) as a white solid. LC/MS (ESI) m/z: 397 (M+H)⁺.

### Step 2: (4-((5-(trifluoromethyl)pyridin-2-yl)oxy)benzoyl)glycine (3)

TFA (2.32 mL) was added to a solution of compound 2 (1.2 g, 3.03 mmol, 1 equiv.) in CH₂Cl₂ (5 mL) at ice-bath temperature. The reaction was stirred at room temperature for 2 hours and then concentrated to dryness to give compound 3 (1.0 g, 2.94 mmol, Yield 97.06%) as white solid. LC/MS (ESI) m/z: 341 (M+H)⁺.

### Step 3: benzyl (1S,3S,5S)-5-methyl-2-((4-((5-(trifluoromethyl)pyridin-2-yl)oxy)benzoyl) glycyl)-2-azabicyclo[3.1.0]hexane-3-carboxylate (4)

To a solution of compound 3 (0.18 g, 0.52 mmol, 1.1 equiv.) in DMF (2 mL) was added benzyl (1S,3S,5S)-5-methyl-2-azabicyclo[3.1.0]hexane-3-carboxylate (0.11 g, 0.48 mmol, 1 equiv.), HATU (0.2 g, 0.53 mmol, 1.3 equiv.) and DIPEA (0.25 mL, 1.43 mmol, 3 equiv.). The reaction was stirred at room temperature for 30 minutes. Water (5 mL) and EtOAc (5 mL) were added. The two layers were separated, and the aqueous layer was extracted with EtOAc (5 mL×2). The combined organic layers were washed with brine, dried over Na₂SO₄, and concentrated. Purification by Combi Flash; 4 g column, solvent A=CH₂Cl₂, solvent B=MeOH, 100% A to 5% B gave compound 4 (0.18 g, 0.33 mmol, Yield 68.38%) as a yellow oil. LC/MS (ESI) m/z: 554 (M+H)⁺.

### Step 4: (1S,3S,5S)-5-methyl-2-((4-((5-(trifluoromethyl)pyridin-2-yl)oxy)benzoyl)glycyl)-2-azabicyclo[3.1.0]hexane-3-carboxylic acid (5)

To a solution of compound 4 (0.18 g, 0.33 mmol, 1 equiv.) in EtOAc (3 mL) was added 10% Pd/C (35 mg, 0.033 mmol, 0.1 equiv.). The flask was evacuated and then backfilled with H₂ in a balloon. The reaction was stirred at room temperature overnight, filtered through Celite pad and the filtrate to concentrated to give compound 5 (0.12 g, 0.26 mmol, Yield 79.69%) as a yellow solid. LC/MS (ESI) m/z: 464 (M+H)⁺.

### Step 5: (1S,3S,5S)-N-((4-carbamimidoylthiophen-2-yl)methyl)-5-methyl-2-((4-((5-(triflu oromethyl)pyridin-2-yl)oxy)benzoyl)glycyl)-2-azabicyclo[3.1.0]hexane-3-carboxamide (Compound 154)

To a solution of compound 5 (120 mg, 0.26 mmol, 1.0 equiv.) in DMF (2 mL) was added 5-(aminomethyl)thiophene-3-carboximidamide. HCl (55 mg, 0.29 mmol, 1.1 equiv.), HATU (108 mg, 0.29 mmol, 1.1 equiv.), and DIPEA (181 μL, 1.04 mmol, 4 equiv.). The reaction was stirred at room temperature for 20 minutes and then purified directly by HPLC to give Compound 154 (40 mg, 0.07 mmol, Yield 26.92%). LC/MS (ESI) m/z: 601 (M+H)⁺. ¹H NMR (400 MHz, DMSO-d₆)¹H NMR (400 MHz, DMSO) δ 8.95 (s, 1H), 8.74 (t, J=5.6 Hz, 1H), 8.70 (s, 1H), 8.58 (s, 1H), 8.55 (t, J=6.0 Hz, 1H), 8.32 (s, 1H), 8.27 (dd, J=8.7, 2.6 Hz, 1H), 7.95 (d, J=8.4 Hz, 2H), 7.46 (s, 1H), 7.40-7.15 (m, 3H), 4.68 (dd, J=11.2, 3.0 Hz, 2H), 4.59-4.29 (m, 4H), 4.07 (dd, J=16.5, 5.8 Hz, 2H), 3.57 (dd, J=17.8, 5.8 Hz, 1H), 3.45 (dd, J=6.0, 2.4 Hz, 1H), 3.07-2.82 (m, 1H), 2.44 (dd, J=13.7, 6.8 Hz, 1H), 2.29 (t, J=12.5 Hz, 2H), (d, J=6.5 Hz, 3H), 0.68 (t, J=5.6 Hz, 2H).

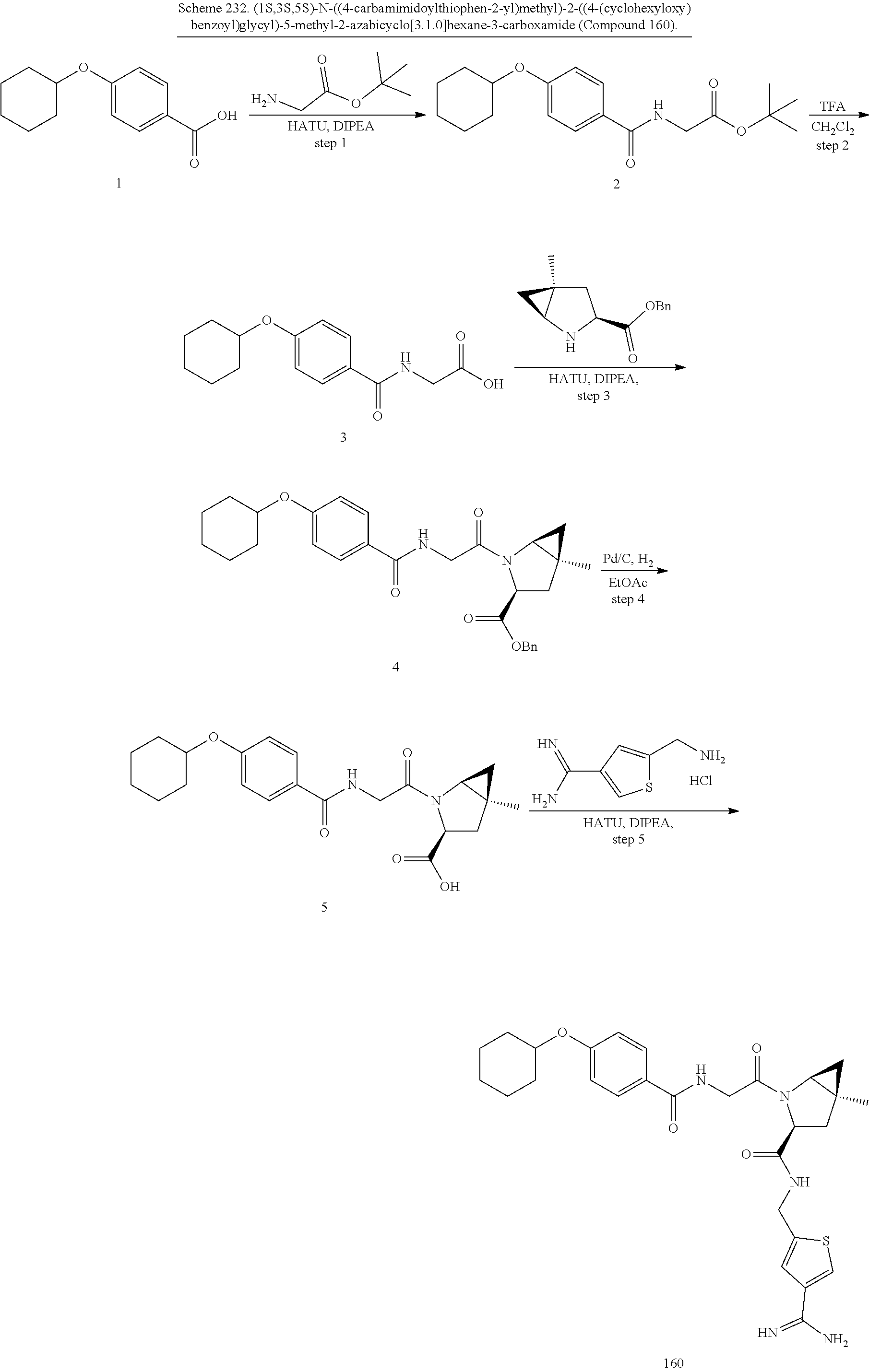

### Step 1: tert-butyl (4-(cyclohexyloxy)benzoyl)glycinate (2)

To a solution of 4-(cyclohexyloxy)benzoic acid, 1 (1 g, 4.54 mmol, 1 equiv.) in DMF (5 mL) was added tert-butyl 2-aminoacetate (0.77 g, 5.90 mmol, 1.3 equiv.), HATU (2.24 g, 5.90 mmol, 1.3 equiv.) and DIPEA (2.38 mL, 13.62 mmol, 3 equiv.). The reaction was stirred at room temperature for 1 h.

Water (10 mL) was added and the solid collected by filtration and then dried to give compound 2 (1.3 g, 3.9 mmol, Yield 85.88%) as a white solid. LC/MS (ESI) m/z: 334 (M+H)⁺.

### Step 2: (4-(cyclohexyloxy)benzoyl)glycine (3)

TFA (2.98 mL) was added to a solution of compound 2 (1.3 g, 3.9 mmol, 1 equiv.) in CH₂Cl₂ (6 mL) at ice-bath temperature. The reaction was stirred at room temperature for 2 hours and then concentrated to dryness. 5 mL of 3 M HCl was added and the mixture concentrated to give compound 3 (1.0 g, 3.61 mmol, Yield 92.49%) as white solid. LC/MS (ESI) m/z: 278 (M+H)⁺.

### Step 3: benzyl (1S,3S,5S)-2-((4-(cyclohexyloxy)benzoyl)glycyl)-5-methyl-2-azabicyclo [3.1.0]hexane-3-carboxylate (4)

To a solution of compound 3 (0.09 g, 0.33 mmol, 1.1 equiv.) in DMF (2 mL) was added benzyl (1S,3S,5S)-5-methyl-2-azabicyclo[3.1.0]hexane-3-carboxylate (0.07 g, 0.30 mmol, 1 equiv.), HATU (0.15 g, 0.39 mmol, 1.3 equiv.) and DIPEA (0.16 mL, 0.91 mmol, 3 equiv.). The reaction was stirred at room temperature for 30 minutes. Water (5 mL) was added. The filtrate was decanted, and the residue was dissolved in EtOAc, washed with brine, dried over Na₂SO₄, and concentrated give compound 4 (0.13 g, 0.27 mmol, Yield 87.56%) as a brown oil. LC/MS (ESI) m/z: 491 (M+H)⁺.

### Step 4: (1S,3S,5S)-2-((4-(cyclohexyloxy)benzoyl)glycyl)-5-methyl-2-azabicyclo[3.1.0]hexane-3-carboxylic acid (5)

To a solution of compound 4 (0.13 g, 0.27 mmol, 1 equiv.) in EtOAc (3 mL) was added 10% Pd/C (28 mg, 0.026 mmol, 0.1 equiv.). The flask was evacuated and then backfilled with H₂ in a balloon. The reaction was stirred at room temperature overnight, filtered through Celite pad and the filtrate concentrated to give compound 5 (0.09 g, 0.23 mmol, Yield 85.19%) as a brown solid. LC/MS (ESI) m/z: 401 (M+H)⁺.

### Step 5: (1S,3S,5S)-N-((4-carbamimidoylthiophen-2-yl)methyl)-2-((4-(cyclohexyloxy) benzoyl)glycyl)-5-methyl-2-azabicyclo[3.1.0]hexane-3-carboxamide (Compound 160)

To a solution of compound 5 (60 mg, 0.15 mmol, 1.0 equiv.) in DMF (2 mL) was added 5-(aminomethyl)thiophene-3-carboximidamide (32 mg, 0.17 mmol, 1.1 equiv.), HATU (63 mg, 0.17 mmol, 1.1 equiv.), and DIPEA (0.11 mL, 0.6 mmol, 4 equiv.). The reaction was stirred at room temperature for 30 minutes and then purified directly by HPLC to give Compound 160 (18 mg, 0.033 mmol, Yield 22.0%). LC/MS (ESI) m/z: 538 (M+H)⁺. ¹H NMR (400 MHz, DMSO-d₆)¹H NMR (400 MHz, DMSO-d₆) δ 8.95 (s, 1H), 8.57-8.43 (m, 1H), 8.34 (d, J=8.2 Hz, 1H), 7.80 (dd, J=9.0, 3.0 Hz, 1H), 7.49 (d, J=13.0 Hz, 1H), 6.98 (dd, J=8.6, 3.6 Hz, 1H), 4.67 (dd, J=11.3, 3.0 Hz, 1H), 4.50-4.25 (m, 2H), 4.01 (td, J=15.9, 5.5 Hz, 1H), 3.45-3.39 (m, 3H), 2.28 (t, J=12.4 Hz, 1H), 2.10-1.87 (m, 6H), 1.79-1.66 (m, 3H), 1.54 (d, J=10.3 Hz, 2H), 1.42 (q, J=11.4 Hz, 4H), 1.16 (s, 3H), 0.90-0.84, (m, 1H), 0.70-0.54 (m, 1H).

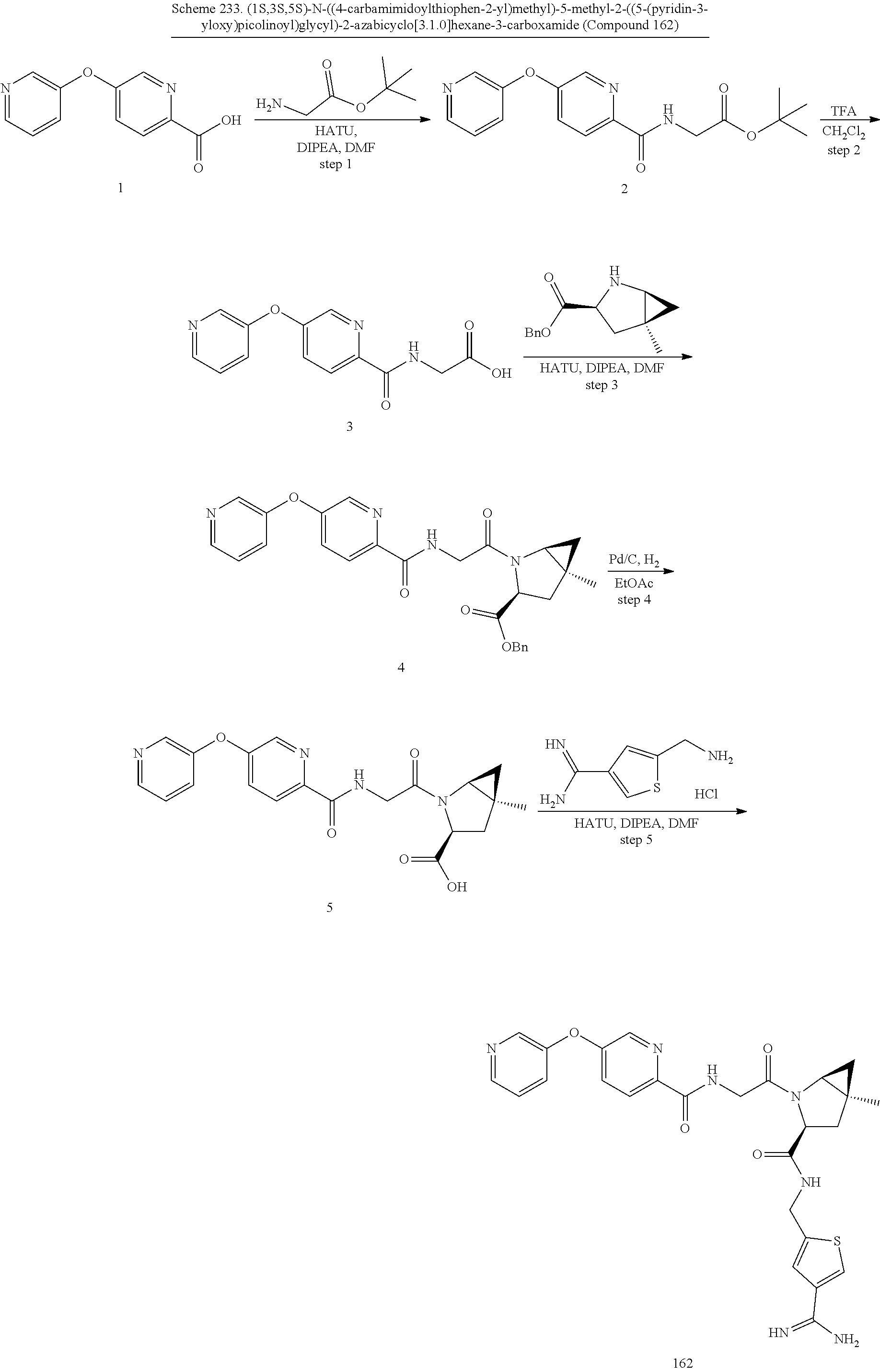

### Step 1: tert-butyl (5-(pyridin-3-yloxy)picolinoyl)glycinate (2)

To a solution of 5-(pyridin-3-yloxy)picolinic acid, 1 (0.5 g, 2.31 mmol, 1 equiv.) in DMF (5 mL) was added tert-butyl 2-aminoacetate. HCl (0.46 g, 2.78 mmol, 1.1 equiv.), HATU (1.32 g, 3.47 mmol, 1.3 equiv.) and DIPEA (1.21 mL, 6.94 mmol, 3 equiv.). The reaction was stirred at room temperature for 1 hour. Water (10 mL) was added and the solid collected by filtration and then dried to give compound 2 (0.46 g, 1.4 mmol, Yield 60.6%) as a white solid. LC/MS (ESI) m/z: 330 (M+H)⁺.

### Step 2: (5-(pyridin-3-yloxy)picolinoyl)glycine (3)

TFA (0.53 mL) was added to a solution of compound 2 (0.23 g, 0.7 mmol, 1 equiv.) in CH₂Cl₂ (2 mL) at ice-bath temperature. The reaction was stirred at room temperature for 2 hours and then concentrated to dryness. 5 mL of 3 M HCl was added and the mixture was concentrated to give compound 3 (0.180 g, 0.66 mmol, Yield 94.29%) as a white solid. LC/MS (ESI) m/z: 274 (M+H)⁺.

### Step 3: benzyl (1S,3S,5S)-5-methyl-2-((5-(pyridin-3-yloxy)picolinoyl)glycyl)-2-azabicyclo [3.1.0]hexane-3-carboxylate (4)

To a solution of compound 3 (0.13 g, 0.43 mmol, 1.1 equiv.) in DMF (2 mL) was added benzyl (1S,3S,5S)-5-methyl-2-azabicyclo[3.1.0]hexane-3-carboxylate (0.09 g, 0.39 mmol, 1 equiv.), HATU (0.19 g, 0.51 mmol, 1.3 equiv.) and DIPEA (0.27 mL, 1.56 mmol, 3 equiv.). The reaction was stirred at room temperature for 30 minutes. Water (5 mL) and EtOAc (5 mL) were added. The two layers were separated, and the aqueous layer was extracted twice with EtOAc. The combined organic layers were washed with brine, dried over Na₂SO₄, and concentrated. Purification by Combi Flash; 4 g column, solvent A=CH₂Cl₂, solvent B=MeOH, 100% A to 5% B gave compound 4 (0.15 g, 0.31 mmol, Yield 72.09%) as a yellow oil. LC/MS (ESI) m/z: 487 (M+H)⁺.

### Step 4: (1S,3S,5S)-5-methyl-2-((5-(pyridin-3-yloxy)picolinoyl)glycyl)-2-azabicyclo [3.1.0]hexane-3-carboxylic acid (5)

To a solution of compound 4 (0.15 g, 0.31 mmol, 1 equiv.) in EtOAc (3 mL) was added 10% Pd/C (33 mg, 0.031 mmol, 0.1 equiv.). The flask was evacuated and then backfilled with H₂ in a balloon. The reaction was stirred at room temperature overnight, filtered through Celite pad and then concentrated to give compound 5 (0.12 g, 0.303 mmol, Yield 97.74%) as a brown solid. LC/MS (ESI) m/z: 397 (M+H)⁺.

### Step 5: (1S,3S,5S)-N-((4-carbamimidoylthiophen-2-yl)methyl)-5-methyl-2-((5-(pyridin-3-yloxy)picolinoyl)glycyl)-2-azabicyclo[3.1.0]hexane-3-carboxamide (Compound 162)

To a solution of compound 5 (50 mg, 0.13 mmol, 1.0 equiv.) in DMF (2 mL) was added 5-(aminomethyl)thiophene-3-carboximidamide (27 mg, 0.14 mmol, 1.1 equiv.), HATU (53 mg, 0.14 mmol, 1.1 equiv.), and DIPEA (88 μL, 0.51 mmol, 4 equiv.). The reaction mixture was stirred at room temperature for 20 minutes and then purified directly by HPLC to give Compound 162 (10 mg, 0.019 mmol, Yield 14.61%). LC/MS (ESI) m/z: 534 (M+H)⁺. ¹H NMR (400 MHz, DMSO-d₆) δ 8.69 (d, J=5.0 Hz, 1H), 8.61 (t, J=5.8 Hz, 1H), 8.58-8.40 (m, 3H), 8.36 (s, 1H), 8.08 (dd, J=8.8, 5.0 Hz, 1H), 7.72-7.56 (m, 2H), 7.58-7.38 (m, 2H), 4.67 (dd, J=11.3, 3.1 Hz, 2H), 4.53-4.16 (m, 2H), 4.06 (dd, J=16.5, 5.8 Hz, 2H), 3.71-3.47 (m, 1H), 2.29 (t, J=12.5 Hz, 2H), 2.00 (dd, J=13.5, 3.1 Hz, 1H), 1.22 (d, J=11.2 Hz, 3H), 0.81-0.60 (m, 2H).

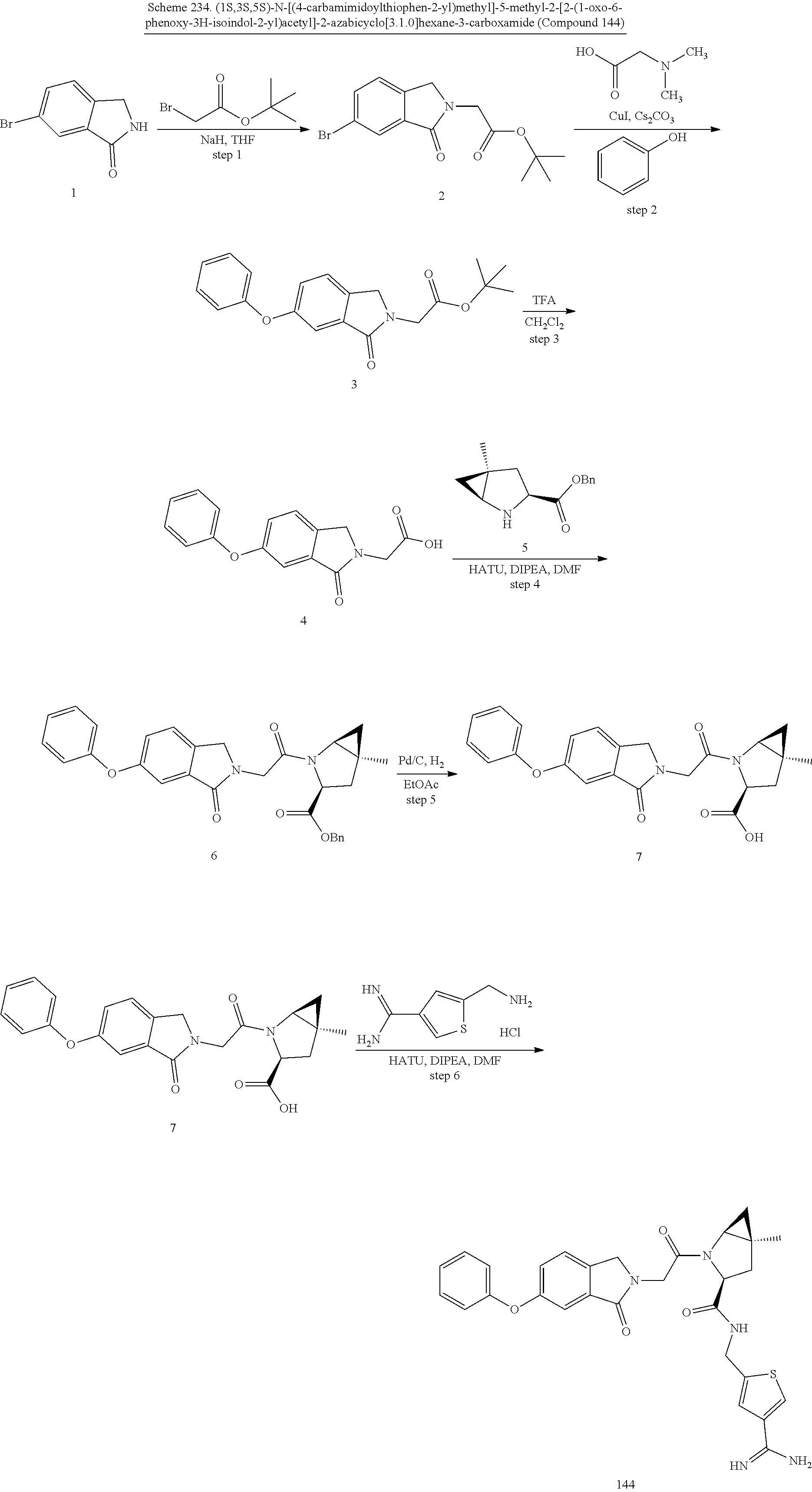

### Step 1: tert-butyl 2-(6-bromo-1-oxoisoindolin-2-yl)acetate (2)

A solution of 6-bromo-2,3-dihydroisoindol-1-one, 1 (1 g, 4.72 mmol, 1.0 equiv.) in THE (15 mL) was added to hexanes washed NaH (0.57 g, 14.15 mmol, 3.0 equiv.) in THE (25 mL) at ice-bath temperature. The mixture was stirred for 30 minutes under argon, tert-butyl 2-bromoacetate (2.76 g, 14.15 mmol, 3.0 equiv.) was then added and the reaction was warmed to room temperature and stirred for 30 minutes. The reaction was quenched with ice-cold water, the two layers were separated, and the aqueous layer was extracted with EtOAc (20 mL×2). The combined organic layers were washed with brine, dried over Na₂SO₄ and concentrated. Purification by Combi Flash; 24 g column solvent A=CH₂Cl₂ solvent B=MeOH, 100% A to 5% B gave compound 2 (1.2 g, 3.68 mmol, Yield 77.97%). LC/MS (ESI) m/z: 327 (M+H)⁺.

### Step 2: tert-butyl 2-(1-oxo-6-phenoxy-3H-isoindol-2-yl)acetate (3)

A mixture of compound 2 (1 g, 3.1 mmol, 1.0 equiv.), phenol (0.43 g, 4.6 mmol, 1.5 equiv.), dimethylaminoaceticacid. HCl (0.13 g, 0.92 mmol, 0.3 equiv.), Cs₂CO₃ (4 g, 12.3 mmol, 4.0 equiv.), and copper(I)iodide (0.06 g, 0.32 mmol, 0.1 equiv.) in dioxane (10 mL) were heated to 90° C. in a sealed tube overnight. The reaction was cooled to room temperature then diluted with water (20 mL) and EtOAc (20 mL). The two layers were separated, and the aqueous layer was extracted with EtOAc (20 mL×2). The combined organic extracts were washed with brine, dried over Na₂SO₄ and concentrated to give a yellow oil. Purification by Combi-Flash; 24 g column, solvent A=hexanes, solvent B=EtOAc, 100% hexanes to 30% EtOAc gave compound 3 (0.74 g, 2.18 mmol, 71.12%) as a yellow oil. LC/MS (ESI) m/z: 340 (M+H)⁺.

### Step 3: (1-oxo-6-phenoxy-3H-isoindol-2-yl)acetic acid (4)

TFA (1.58 mL) was added to a solution of compound 3 (0.35 g, 1.03 mmol, 1.0 equiv.) in CH₂Cl₂ (3 mL) at ice-bath temperature. The reaction was stirred at room temperature for 2 hours and then concentrated to dryness. 5 mL of 3N HCl was added and the mixture concentrated again to dryness to give compound 4 (0.27 g, 0.953 mmol, Yield 92.42%) as an off yellow solid. LC/MS (ESI) m/z: 284 (M+H)⁺.

### Step 4: benzyl (1S,3S,5S)-5-methyl-2-[2-(1-oxo-6-phenoxy-3H-isoindol-2-yl)acetyl]-2-azabicyclo[3.1.0]hexane-3-carboxylate (6)

To a solution of compound 4 (0.14 g, 0.48 mmol, 1.0 equiv.) in DMF (2 mL) was added benzyl (1S,3S,5S)-5-methyl-2-azabicyclo[3.1.0]hexane-3-carboxylate (5) (0.1 g, 0.43 mmol, 1.0 equiv.), HATU (0.21 g, 0.56 mmol, 1.3 equiv.) and DIPEA (0.23 mL, 1.3 mmol, 3.0 equiv.). The reaction was stirred at room temperature for 30 minutes. Water (5 mL) and EtOAc (5 mL) were added. The two layers were separated, and the aqueous layer was extracted with EtOAc (5 mL×2), washed with brine, dried over Na₂SO₄, and concentrated. Purification by Combi Flash, 4 g column, solvent A=CH₂Cl₂, solvent B=MeOH, 100% A to 5% B gave compound 6 (0.2 g, 0.40 mmol, Yield 93.16%) as a yellow oil. LC/MS (ESI) m/z: 497 (M+H)⁺.

### Step 5: 1S,3S,5S)-5-methyl-2-[2-(1-oxo-6-phenoxy-3H-isoindol-2-yl)acetyl]-2-azabicyclo[3.1.0]hexane-3-carboxylic acid (7)

To a solution of compound 6 (100 mg, 0.20 mmol, 1.0 equiv.) in EtOAc (3 mL) added 5% Pd/C (43 mg, 0.02 mmol, 0.1 equiv.). The flask was evacuated and then backfilled with H₂ in a balloon. The reaction was stirred at room temperature for 1 hour and then filtered through Celite pad. The filtrate was concentrated and dried in vacuo to give compound 7 (75 mg, 0.18 mmol, Yield 90.0%) as a yellow solid. LC/MS (ESI) m/z: 407 (M+H)⁺.

### Step 6: (1S,3S,5S)-N-[(4-carbamimidoylthiophen-2-yl)methyl]-5-methyl-2-[2-(1-oxo-6-phenoxy-3H-isoindol-2-yl)acetyl]-2-azabicyclo[3.1.0]hexane-3-carboxamide (Compound 144)

To a solution of compound 7 (70 mg, 0.17 mmol, 1.0 equiv.) in DMF (2 mL) was added 5-(aminomethyl)thiophene-3-carboximidamide. HCl (29 mg, 0.19 mmol, 1.1 equiv.), HATU (72 mg, 0.19 mmol, 1.1 equiv.), and DIPEA (0.12 mL, 0.69 mmol, 4.0 equiv.). The reaction mixture was stirred at room temperature for 30 minutes and then purified directly by HPLC to give Compound 144 (20 mg, 0.04 mmol, Yield 23.53%). LC/MS (ESI) m/z: 544 (M+H)⁺. ¹H NMR (400 MHz, DMSO-d₆) δ 8.96 (s, 1H), 8.62 (t, J=5.8 Hz, 1H), 8.45 (s, 1H), 8.32 (d, J=6.1 Hz, 1H), 7.63 (t, J=6.5 Hz, 1H), 7.50 (s, 1H), 7.47-7.39 (m, 2H), 7.31 (dd, J=8.2, 2.4 Hz, 1H), 7.24-7.11 (m, 2H), 7.07 (d, J=8.0 Hz, 2H), 4.86 (d, J=10.9 Hz, 1H), 4.69-4.60 (m, 1H), 4.57 (d, J=9.3 Hz, 1H), 4.54-4.35 (m, 2H), 4.33 (dd, J=15.4, 5.7 Hz, 1H), 4.21 (d, J=3.9 Hz, 1H), 3.41 (dd, J=6.0, 2.4 Hz, 1H), 2.29 (t, J=12.6 Hz, 1H), 1.95 (dd, J=13.2, 3.2 Hz, 1H), 1.21 (s, 3H), 0.66-0.60 (m, 2H).

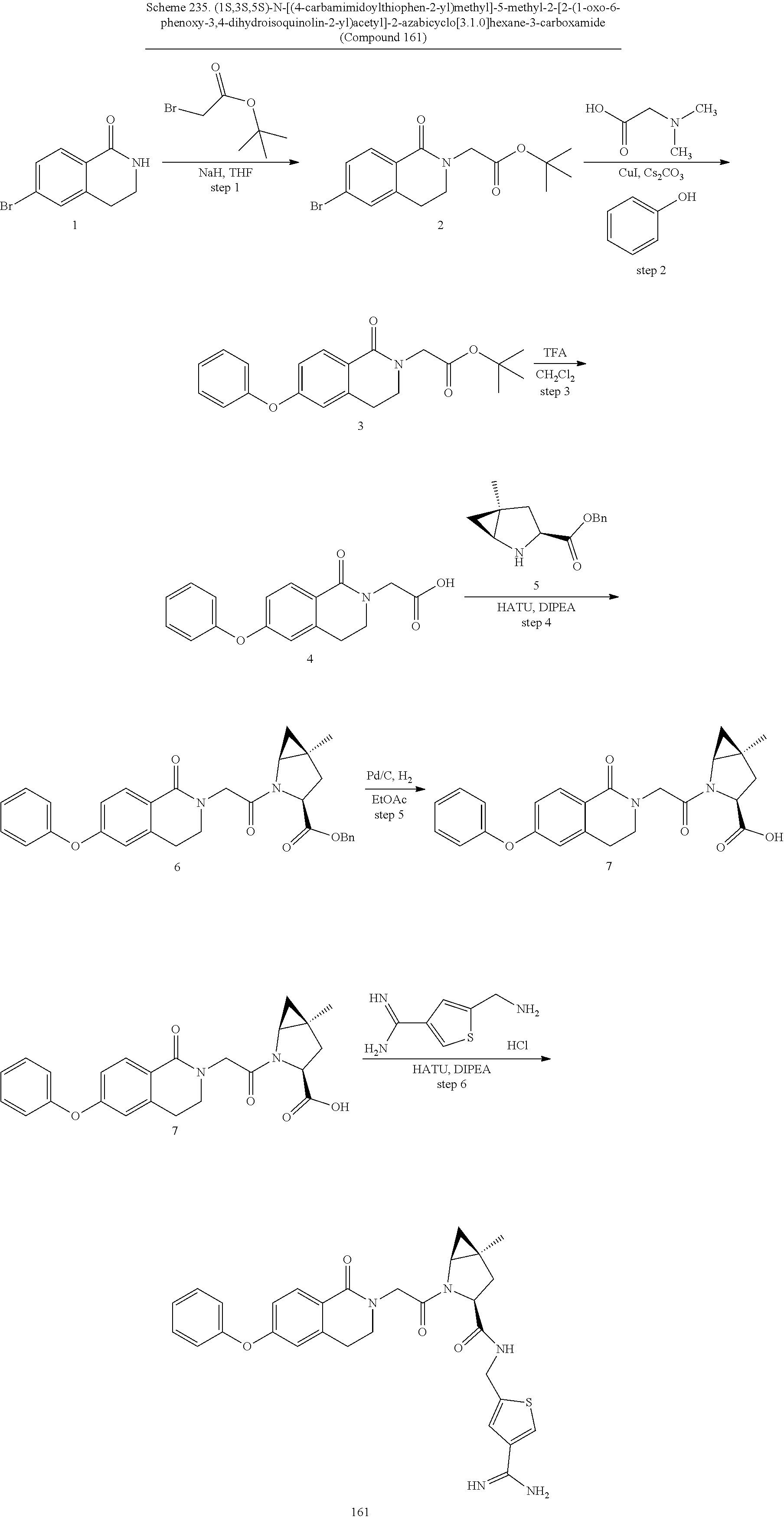

### Step 1: tert-butyl 2-(6-bromo-1-oxo-3,4-dihydroisoquinolin-2-yl)acetate (2)

A solution of 6-bromo-3,4-dihydro-2H-isoquinolin-1-one, 1 (1 g, 4.42 mmol, 1.0 equiv.) in THE (15 mL) was added to hexanes washed NaH (0.53 g, 13.27 mmol, 3.0 equiv.) in THE (25 mL) at ice-bath temperature. The mixture was stirred for 30 minutes under argon, tert-butyl 2-bromoacetate (2.59 g, 13.27 mmol, 3.0 equiv.) was then added and the reaction was warmed to room temperature and stirred for 1 h. The reaction was quenched with ice-cold water, the two layers were separated, and the aqueous layer was extracted with EtOAc. The combined organic layers were washed with brine, dried over Na₂SO₄ and concentrated to give compound 2 (1.2 g, 3.53 mmol, Yield 79.74%) as a brown powder. LC/MS (ESI) m/z: 341 (M+H)⁺.

### Step 2: tert-butyl 2-(1-oxo-6-phenoxy-3,4-dihydroisoquinolin-2-yl)acetate (3)

A mixture of compound 2 (1 g, 2.94 mmol, 1.0 equiv.), phenol (0.83 g, 8.82 mmol, 3 equiv.), dimethylaminoacetic acid. HCl (123 mg, 0.88 mmol, 0.3 equiv.), Cs₂CO₃ (3.83 g, 11.76 mmol, 4.0 equiv.), and copper(I)iodide (56 mg, 0.29 mmol, 0.1 equiv.) in dioxane (20 mL) were heated to 90° C. in a sealed tube overnight. The reaction was cooled to room temperature then diluted with water (20 mL) and EtOAc (20 mL). The two layers were separated, and the aqueous layer was extracted with EtOAc (25 mL×2). The combined organic extracts were washed with brine, dried over Na₂SO₄ and concentrated to give compound 3 (0.95 g, 2.69 mmol, 91.54%) as a brown oil. LC/MS (ESI) m/z: 354 (M+H)⁺.

### Step 3: (1-oxo-6-phenoxy-3,4-dihydroisoquinolin-2-yl)acetic acid (4)

TFA (2.06 mL) was added to a solution of compound 3 (0.95 g, 2.69 mmol, 1.0 equiv.) in CH₂Cl₂ (5 mL) at ice-bath temperature. The reaction was stirred at room temperature for 2 hours and then concentrated to dryness to give compound 4 (0.7 g, 2.36 mmol, Yield 87.62%) as pale yellow solid which was used in the next step without further purification. LC/MS (ESI) m/z: 298 (M+H)⁺.

### Step 4: benzyl (1S,3S,5S)-5-methyl-2-[2-(1-oxo-6-phenoxy-3,4-dihydroisoquinolin-2-yl)acetyl]-2-azabicyclo[3.1.0]hexane-3-carboxylate (6)

To a solution of compound 4 (0.11 g, 0.38 mmol, 1.1 equiv.) in DMF (2 mL) was added benzyl (1S,3S,5S)-5-methyl-2-azabicyclo[3.1.0]hexane-3-carboxylate (5) (0.08 g, 0.35 mmol, 1.0 equiv.), HATU (0.17 g, 0.45 mmol, 1.3 equiv.) and DIPEA (0.18 mL, 1.04 mmol, 3.0 equiv.). The reaction was stirred at room temperature for 30 minutes. Water (5 mL) was added and the liquid was decanted and the residue taken up in EtOAc, washed with brine, dried over Na₂SO₄, and concentrated to give compound 6 (0.15 g, 0.29 mmol, Yield 84.03%) as a brown oil which was used in the next reaction without further purification. LC/MS (ESI) m/z: 511 (M+H)⁺.

### Step 5: (1S,3S,5S)-5-methyl-2-[2-(1-oxo-6-phenoxy-3,4-dihydroisoquinolin-2-yl)acetyl]-2-azabicyclo [3.1.0]hexane-3-carboxylic acid (7)

To a solution of compound 6 (0.13 g, 0.25 mmol, 1.0 equiv.) in EtOAc (3 mL) added 10% Pd/C (27 mg, 0.025 mmol, 0.1 equiv.). The flask was evacuated and then backfilled with H₂ in a balloon. The reaction was stirred at room temperature overnight and then filtered through Celite pad. The filtrate was concentrated and dried in vacuo to give compound 7 (0.09 g, 0.21 mmol, Yield 84.0%) as a yellow solid. LC/MS (ESI) m/z: 421 (M+H)⁺.

### Step 6: (1S,3S,5S)-N-((4-carbamimidoylthiophen-2-yl)methyl)-5-methyl-2-(2-(1-oxo-6-phenoxy-3,4-dihydroisoquinolin-2(1H)-yl)acetyl)-2-azabicyclo[3.1.0]hexane-3-carboxamide (Compound 162)

To a solution of compound 7 (90 mg, 0.21 mmol, 1.0 equiv.) in DMF (2 mL) was added 5-(aminomethyl)thiophene-3-carboximidamide. HCl (45 mg, 0.24 mmol, 1.1 equiv.), HATU (90 mg, 0.24 mmol, 1.1 equiv.), and DIPEA (0.15 mL, 0.86 mmol, 4.0 equiv.). The reaction mixture was stirred at room temperature for 30 minutes and then purified directly by HPLC to give Compound 162 (30 mg, 0.054 mmol, Yield 25.71%). LC/MS (ESI) m/z: 558 (M+H). ¹H NMR (400 MHz, DMSO-d₆) δ 8.98 (s, 1H), 8.60 (t, J=5.8 Hz, 1H), 8.38 (d, J=9.5 Hz, 1H), 7.87 (d, J=8.5 Hz, 1H), 7.51-7.43 (m, 2H), 7.24 (t, J=7.4 Hz, 1H), 7.13 (d, J=7.9 Hz, 2H), 6.97-6.86 (m, 2H), 4.68 (dd, J=11.3, 3.1 Hz, 1H), 4.59 (d, J=16.3 Hz, 1H), 4.54-4.32 (m, 4H), 3.58 (t, J=6.7 Hz, 2H), 3.39 (d, J=3.6 Hz, 1H), 2.96 (dq, J=15.9, 7.1 Hz, 2H), 2.53 (d, J=2.6 Hz, 2H), 2.30 (t, J=12.4 Hz, 1H), 2.00 (dd, J=13.3, 3.1 Hz, 1H), 1.25 (s, 3H), 1.23 (d, J=4.7 Hz, 1H), 0.66 (q, J=8.0 Hz, 1H).

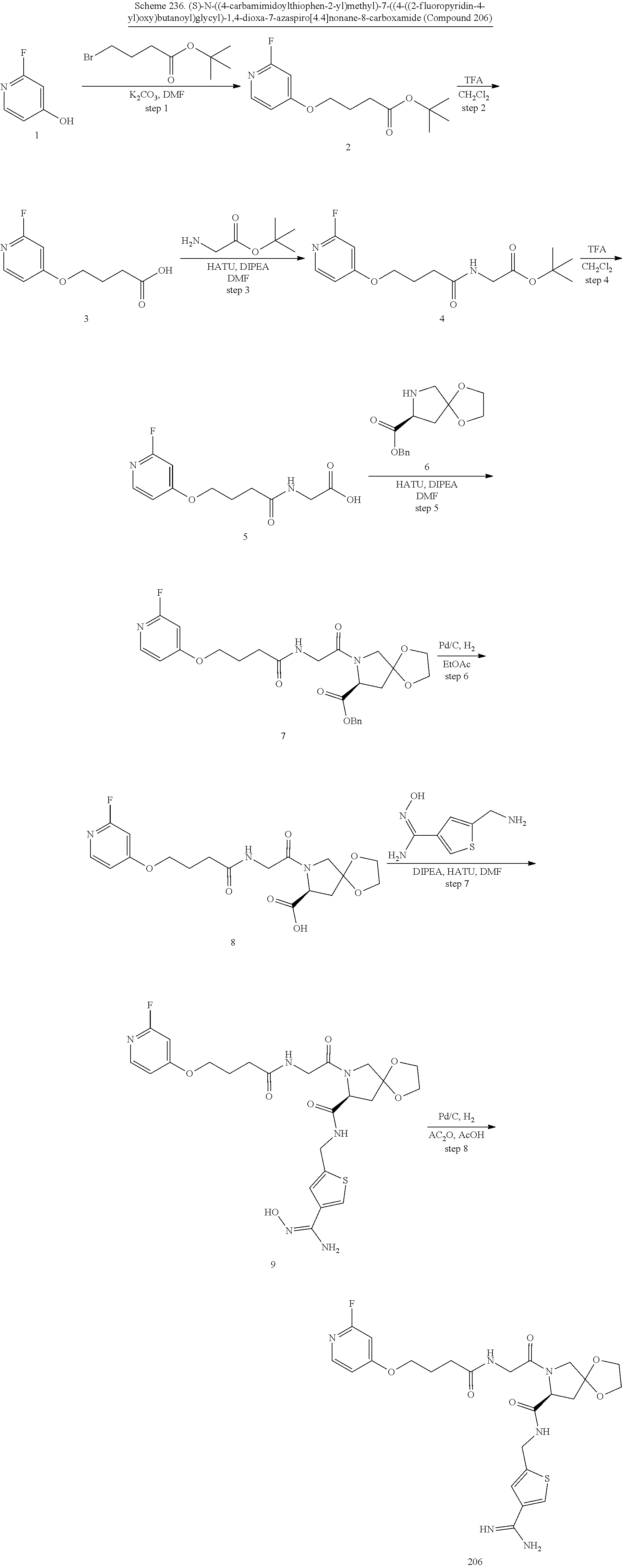

### Step 1: 4-((2-fluoropyridin-4-yl)oxy)butanoic acid (2)

To a solution of 2-fluoropyridin-4-ol, 1 (0.71 g, 6.28 mmol, 2 equiv.) in DMF (5 ml) was added tert-butyl 4-bromobutanoate (0.7 g, 3.14 mmol 1.0 equiv.), and K₂CO₃ (1.08 g, 7.84 mmol, 2.5 equiv.). The reaction was stirred for 24 hours at room temperature under argon and then diluted with water (10 mL) and EtOAc (10 mL). The two layers were separated, and the aqueous layer was extracted with EtOAc (10 mL×2). The combined organic extracts were washed with brine, dried over Na₂SO₄, and concentrated. Purification by Combi Flash; 24 g column, solvent A=Hexanes, solvent B=EtOAc, 100% A to 20% B gave compound 2 (0.75 g, 2.94 mmol, 46.81% Yield). LC/MS (ESI) m/z: 256 (M+H)⁺.

### Step 2: 4-((2-fluoropyridin-4-yl)oxy)butanoic acid (3)

TFA (2.22 mL) was added to a solution of compound 2 (0.75 g, 2.94 mmol, 1.0 equiv.) in CH₂Cl₂ (5 mL). The reaction was stirred at room temperature for 3 hours and then concentrated to give compound 3 (0.55 g, 2.76 mmol, 93.88% Yield) as a white solid. LC/MS (ESI) m/z: 200 (M+H)+

### Step 3: tert-butyl (4-((2-fluoropyridin-4-yl)oxy)butanoyl)glycinate (4)

To a solution of compound 3 (0.4 g, 2.01 mmol, 1.0 equiv.) in DMF (5 mL) was added glycine tert-butyl ester. HCl (0.40 g, 2.41 mmol, 1.2 equiv.), HATU (1 g, 2.63 mmol, 1.3 equiv.), and finally DIPEA (0.88 mL, 5.02 mmol, 3.0 equiv.). The reaction was stirred for 20 minutes at room temperature and then diluted with water (10 mL) and CH₂Cl₂ (10 mL). The two layers were separated, and the aqueous layer was extracted with CH₂Cl₂ (10 mL×3). The combined organic layers, were washed brine, dried over Na₂SO₄ and concentrated to give a yellow oil. Purification by Combi Flash; 24 g column, solvent A=CH₂Cl₂, solvent B=MeOH, 100% A to 5% B gave compound 4 (0.5 g, 1.60 mmol, 79.73% Yield). LC/MS (ESI) m/z: 313 (M+H)+

### Step 4: (4-((2-fluoropyridin-4-yl)oxy)butanoyl)glycine (5)

TFA (1.0 mL) was added to a solution of compound 4 (0.5 g, 1.6 mmol, 1.0 equiv.) in CH₂Cl₂ (2 mL). The reaction mixture was stirred at room temperature for 2 hours and then concentrated. 4 M HCl (5 mL) was added and the mixture concentrated and dried in vacuo to afford compound 5 (0.38 g, 1.48 mmol, 92.77% Yield) as a white solid. LC/MS (ESI) m/z: 257 (M+H)⁺.

### Step 5: benzyl (S)-7-((4-((2-fluoropyridin-4-yl)oxy)butanoyl)glycyl)-1,4-dioxa-7-azaspiro [4.4]nonane-8-carboxylate (6)

To a mixture of compound 5 (96.03 mg, 0.38 mmol, 1.1 equiv.) and benzyl (8S)-1,4-dioxa-7-azaspiro[4.4]nonane-8-carboxylate (90 mg, 0.34 mmol, 1.0 equiv.) in DMF (2 mL) was added DIPEA (0.18 mL, 1.02 mmol, 3 equiv.) and HATU (168 mg, 0.44 mmol, 1.3 equiv.). The reaction was stirred at room temperature for 1 hour. The mixture was diluted with H₂O and extracted with CH₂Cl₂ twice. The combined organic extracts were dried over anhydrous Na₂SO₄, filtered, and concentrated to dryness. Purification by Combi Flash; 12 g column, solvent A=CH₂Cl₂, solvent B=MeOH, 100% A to 5% B gave compound 6 (120 mg, 0.24 mmol, 70.44% Yield) as white sticky solid. LC/MS (ESI) m/z: 502 (M+H)+

### Step 6: (S)-7-((4-((2-fluoropyridin-4-yl)oxy)butanoyl)glycyl)-1,4-dioxa-7-azaspiro[4.4]nonane-8-carboxylic acid (7)

To a solution of compound 6 (120 mg, 0.24 mmol, 1.0 equiv.) in EtOAc (3 mL) was added 10% Pd/C (25 mg, 0.024 mmol, 0.1 equiv.). The flask was evacuated and backfilled with H₂ in a balloon and stirred under H₂ atmosphere for 12 hours. The mixture was filtered through Celite pad, and the filtrate was concentrated to dryness under reduced pressure to give compound 7 (90 mg, 0.22 mmol, Yield 91.67%) as yellow oil, which was used directly in the next step. LC/MS (ESI) m/z: 412 (M+H)⁺.

### Step 7: (S,E)-7-((4-((2-fluoropyridin-4-yl)oxy)butanoyl)glycyl)-N-((4-(N′-hydroxycarbamimid oyl)thiophen-2-yl)methyl)-1,4-dioxa-7-azaspiro[4.4]nonane-8-carboxamide (8)

To a mixture of compound 7 (90 mg, 0.22 mmol, 1.0 equiv.) and [[5-(aminomethyl) thiophene-3-carboximidoyl]amino]acetate. HCl (80 mg, 0.3 mmol, 1.1 equiv.) in DMF (2 mL) was added DIPEA (0.204 mL, 1.17 mmol, 4 equiv.), and HATU (122 mg, 0.32 mmol, 1.1 equiv.). The mixture was stirred at room temperature for 1 hour. The mixture was diluted with H₂O and extracted with CH₂Cl₂ (10 mL×3). The combined organic extracts were dried over anhydrous Na₂SO₄, filtered, and concentrated. Purification by Combi Flash; 12 g column, solvent A=CH₂Cl₂, solvent B=MeOH, 100% A to 10% B % gave compound 8 (25 mg, 0.041 mmol, Yield 18.75%). LC/MS (ESI) m/z: 607 (M+H)⁺.

### Step 8: (S)-N-((4-carbamimidoylthiophen-2-yl)methyl)-7-((4-((2-fluoropyridin-4-yl)oxy) but anoyl)glycyl)-1,4-dioxa-7-azaspiro[4.4]nonane-8-carboxamide (Compound 206)

Into a 10 mL round-bottomed flask equipped with a Teflon-coated magnetic stirring bar was added compound 8 (25.0 mg, 0.041 mmol, 1.0 equiv.), acetic acid (0.5 mL) and acetic anhydride (8.0 μL, 0.08 mmol, 2.0 equiv.). The resulting solution was stirred at room temperature for 10 minutes. 10% Pd/C (4.4 mg, 0.0041 mmol, 0.1 equiv.) was added and the reaction mixture. The flask was evacuated and backfilled H₂ in a balloon and stirred at room temperature for 14 hours. The reaction was filtered through Celite pad, and the filtrate was concentrated and then purified directly on HPLC to yield Compound 206 (2.5 mg, 0.005 mmol, Yield 12.2%). LC/MS (ESI) m/z: 549 (M+H)⁺. ¹H NMR (400 MHz, DMSO-d₆) δ 9.02 (s, 1H), 8.55 (dt, J=19.0, 5.8 Hz, 2H), 8.45 (s, 1H), 8.36-8.29 (m, 1H), 7.83 (dd, J=9.0, 2.5 Hz, 2H), 7.55-7.30 (m, 2H), 7.08 (dd, J=9.1, 2.8 Hz, 2H), 5.17 (s, 2H), 4.71-4.57 (m, 1H), 4.50-4.40 (m, 1H), 4.43-4.33 (m, 4H), 4.31 (dd, J=16.6, 5.6 Hz, 1H), 4.02 (td, J=16.2, 5.6 Hz, 1H), 3.43 (dd, J=6.1, 2.4 Hz, 2H), 2.32-2.26 (m, 2H), 2.11-1.89 (m, 2H)

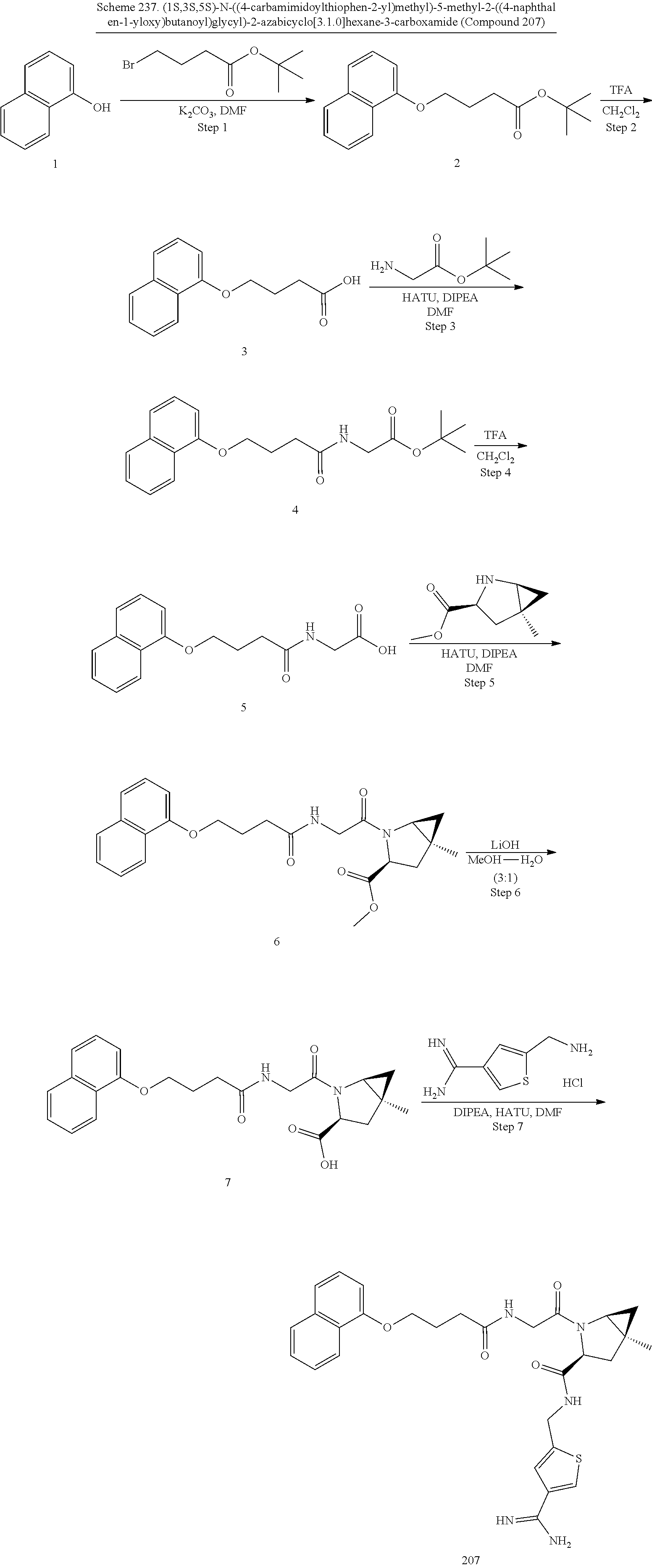

### Step 1: tert-butyl 4-(naphthalen-1-yloxy)butanoate (2)

To a solution of naphthalen-1-ol, 1 (905 mg, 6.28 mmol, 2 equiv.) in DMF (5 mL) was added, tert-butyl 4-bromobutanoate (0.70 g, 3.14 mmol 1.0 equiv.), and K₂CO₃ (1.08 g, 7.84 mmol, 2.5 equiv.). The reaction was stirred for 24 hours at room temperature under argon and then diluted with water (10 mL) and EtOAc (10 mL). The two layers were separated, and the aqueous layer was extracted with EtOAc (10 mL×2). The combined organic extracts were washed with brine, dried over Na₂SO₄, and concentrated. Purification by Combi Flash; 24 g column, solvent A=Hexanes, solvent B=EtOAc, 100% A to 20% B gave compound 2 (0.79 g, 2.76 mmol, 87.56% Yield). LC/MS (ESI) m/z: 287 (M+H)⁺.

### Step 2: 4-(naphthalen-1-yloxy)butanoic acid (3)

TFA (1.70 mL) was added to a solution of compound 2 (0.65 g, 2.27 mmol, 1.0 equiv.) in CH₂Cl₂ (3 mL) at ice-bath temperature. The reaction was stirred at room temperature for 3 hours and then concentrated to dryness to give compound 3 (0.5 g, 2.17 mmol, Yield 95.66%) as an off yellow solid which was used in the next step without further purification. LC/MS (ESI) m/z: 231 (M+H)⁺.

### Step 3: tert-butyl (4-(naphthalen-1-yloxy)butanoyl)glycinate (4)

To a solution of compound 3 (0.5 g, 2.17 mmol, 1 equiv.) in DMF (5 mL) was added tert-butyl 2-aminoacetate (0.54 g, 3.26 mmol, 1.5 equiv.), HATU (1.24 g, 3.26 mmol, 1.5 equiv.) and DIPEA (1.14 mL, 6.51 mmol, 3 equiv.). The reaction was stirred at room temperature for 1 hour. Water (10 mL) was added and the solid collected by filtration and then dried to give compound 4 (0.6 g, 1.75 mmol, Yield 80.61%) as a white solid. LC/MS (ESI) m/z: 344 (M+H)⁺.

### Step 4: (4-(naphthalen-1-yloxy)butanoyl)glycine (5) TFA (1.0 mL) was added to a solution of compound 4 (0.50 g, 1.46 mmol, 1.0 equiv.) in CH₂Cl₂ (3 mL) at ice-bath temperature. The reaction was stirred at room temperature for 2 hours and then concentrated to dryness to give compound 5 (0.39 g, 1.36 mmol, Yield 93.07%) as white solid which was used in the next step without further purification. LC/MS (ESI) m/z: 288 (M+H)⁺

### Step 5: methyl (1S,3S,5S)-5-methyl-2-((4-(naphthalen-1-yloxy)butanoyl)glycyl)-2-azabicyclo [3.1.0]hexane-3-carboxylate (6)

To a solution of compound 5 (0.14 g, 0.5 mmol, 1.1 equiv.) in DMF (2 mL) was added methyl (1S,3S,5S)-5-methyl-2-azabicyclo[3.1.0]hexane-3-carboxylate (0.07 g, 0.45 mmol, 1.0 equiv.), HATU (0.22 g, 0.59 mmol, 1.3 equiv.), and DIPEA (0.24 mL, 1.35 mmol, 3.0 equiv.). The reaction mixture was stirred at room temperature for 30 minutes and then Water (5 mL) was added and the liquid was decanted. The residue taken up in EtOAc, washed with brine, dried over Na₂SO₄, and concentrated to give to give compound 6 (0.12 g, 0.283 mmol, 62.89% Yield) as a brown oil. LC/MS (ESI) m/z: 425 (M+H)⁺.

### Step 6: (1S,3S,5S)-5-methyl-2-((4-(naphthalen-1-yloxy)butanoyl)glycyl)-2-azabicyclo [3.1.0]hexane-3-carboxylic acid (7)

To a solution of compound 6 (0.18 g, 0.42 mmol, 1.0 equiv.) in MeOH (3 mL) water (1 mL) was added LiOH (11 mg, 0.45 mmol, 1.1 equiv.). The reaction was stirred at room temperature overnight and concentrated to remove MeOH and then diluted with EtOAc. The pH was adjusted to 1 using 1 N HCl.

The two layers were separated, and the aqueous layer was extracted with EtOAc (5 mL×2). The combined organic extracts were washed with brine, dried over Na₂SO₄ and concentrated to give compound 7 (0.16 g, 0.39 mmol, 92.0% Yield) as a white solid. LC/MS (ESI) m/z: 411 (M+H)⁺.

### Step 7: (1S,3S,5S)-N-((4-carbamimidoylthiophen-2-yl)methyl)-5-methyl-2-((4-(naphthalen-1-yloxy)butanoyl)glycyl)-2-azabicyclo[3.1.0]hexane-3-carboxamide (Compound 207)

To a solution of compound 7 (30 mg, 0.073 mmol, 1.0 equiv.) in DMF (2 mL) was added 5-(aminomethyl)thiophene-3-carboximidamide. HCl (17 mg, 0.09 mmol, 1.2 equiv.), HATU (33 mg, 0.089 mmol, 1.2 equiv.), and DIPEA (0.04 mL, 0.23 mmol, 3.0 equiv.). The reaction mixture was stirred at room temperature for 30 minutes and then purified directly by HPLC to give Compound 207 (0.02 g, 0.04 mmol, Yield 50.0%). LC/MS (ESI) m/z: 548 (M+H)⁺. ¹H NMR (400 MHz, DMSO-d₆) δ 9.05 (s, 1H), 8.55 (dt, J=19.0, 5.8 Hz, 2H), 8.45 (s, 1H), 8.36-8.29 (m, 1H), 7.83 (dd, J=9.0, 2.5 Hz, 2H), 7.55-7.30 (m, 6H), 7.08 (dd, J=9.1, 2.8 Hz, 1H), 5.17 (s, 1H), 4.71-4.57 (m, 2H), 4.50-4.40 (m, 1H), 4.43-4.33 (m, 2H), 4.31 (dd, J=16.6, 5.6 Hz, 1H), 4.02 (td, J=16.2, 5.6 Hz, 1H), 3.43 (dd, J=6.1, 2.4 Hz, 1H), 2.28 (t, J=12.3 Hz, 1H), 2.00 (dd, J=13.2, 3.1 Hz, 1H), 1.91 (s, 3H), 0.70-0.58 (m, 2H).

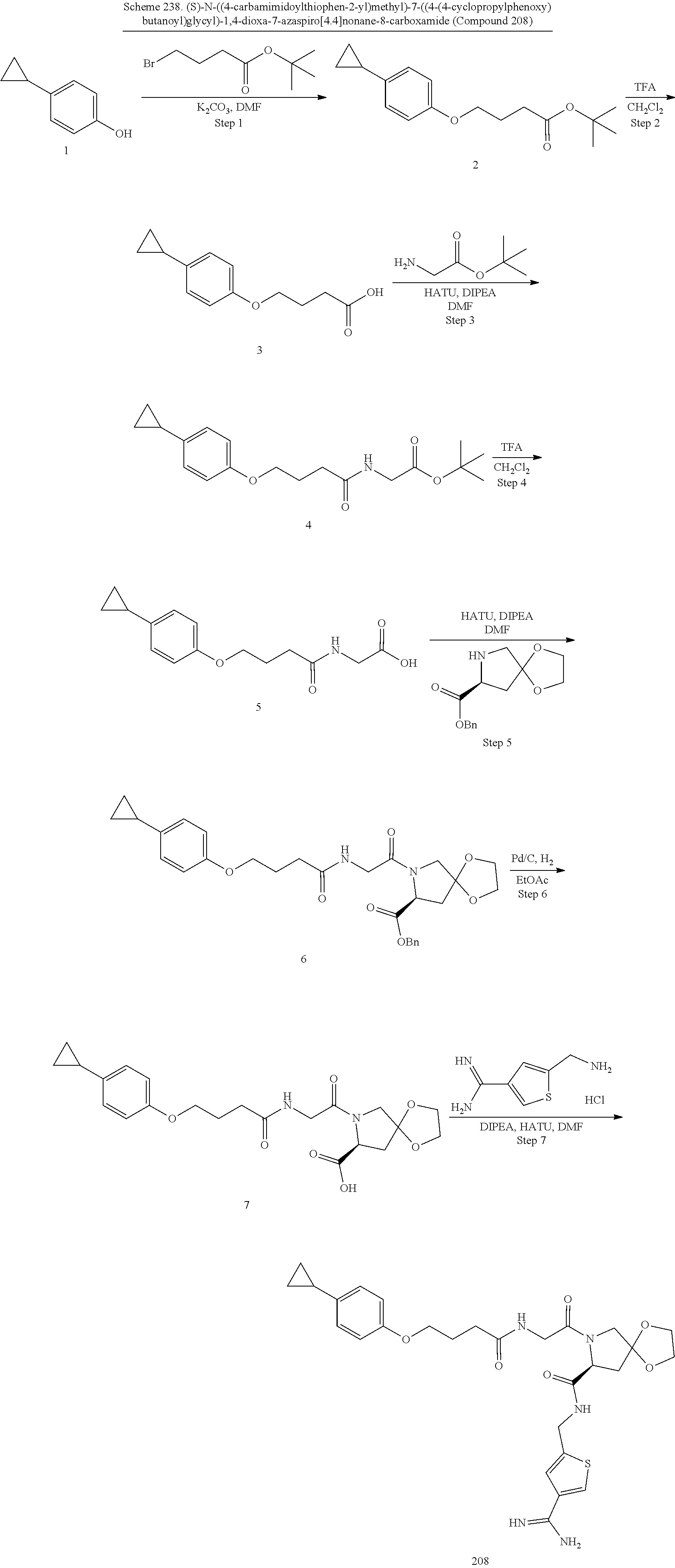

### Step 1: tert-butyl 4-(4-cyclopropylphenoxy)butanoate (2)

To a solution of 4-cyclopropylphenol, 1 (0.6 g, 4.0 mmol, 2 equiv.) in DMF (5 mL) was added, tert-butyl 4-bromobutanoate (0.7 g, 3.0 mmol 1.0 equiv.), and K₂CO₃ (1.0 g, 7.24 mmol, 2.5 equiv.). The reaction was stirred for 24 hours at room temperature under argon and then diluted with water (10 mL) and EtOAc (10 mL). The two layers were separated, and the aqueous layer was extracted with EtOAc (10 mL×2). The combined organic extracts were washed with brine, dried over Na₂SO₄, and concentrated. Purification by Combi Flash; 24 g column, solvent A=Hexanes, solvent B=EtOAc, 100% A to 20% B gave compound 2 (0.80 g, 3.00 mmol, 90.0% Yield). LC/MS (ESI) m/z: 277 (M+H)⁺. as a white solid.

### Step 2: 4-(4-cyclopropylphenoxy)butanoic acid (3)

TFA (1.0 mL) was added to a solution of compound 2 (0.5 g, 1.81 mmol, 1.0 equiv.) in CH₂Cl₂ (2 mL) at ice-bath temperature. The reaction was stirred at room temperature for 3 hours and then concentrated to dryness to give compound 3 (0.38 g, 1.73 mmol, Yield 95.35%) as an off yellow solid which was used in the next step without further purification. LC/MS (ESI) m/z: 221 (M+H)⁺.

### Step 3: tert-butyl (4-(4-cyclopropylphenoxy)butanoyl)glycinate (4)

To a solution of compound 3 (0.3 g, 1.36 mmol, 1 equiv.) in DMF (5 mL) was added tert-butyl 2-aminoacetate (0.30 g, 1.77 mmol, 1.3 equiv.), HATU (0.80 g, 2.10 mmol, 1.5 equiv.) and DIPEA (0.71 mL, 4.1 mmol, 3 equiv.). The reaction was stirred at room temperature for 1 hour. Water (5 mL) was added and the solid collected by filtration and then dried to give compound 4 (0.4 g, 1.0 mmol, Yield 90.0%). LC/MS (ESI) m/z: 334 (M+H)⁺.

### Step 4: (4-(4-cyclopropylphenoxy)butanoyl)glycine (5)

TFA (0.9 mL) was added to a solution of compound 4 (0.4 g, 1.20 mmol, 1.0 equiv.) in CH₂Cl₂ (1 mL) at ice-bath temperature. The reaction was stirred at room temperature for 2 hours and then concentrated to dryness to give compound 5 (0.29 g, 1.05 mmol, Yield 87.2%) as a white solid which was used in the next step without further purification. LC/MS (ESI) m/z: 278 (M+H)⁺.

### Step 5: benzyl (S)-7-((4-(4-cyclopropylphenoxy)butanoyl)glycyl)-1,4-dioxa-7-azaspiro[4.4]nonane-8-carboxylate (6)

To a solution of compound 5 (0.15 g, 0.54 mmol, 1.0 equiv.) in DMF (2 mL) was added benzyl (8S)-1,4-dioxa-7-azaspiro[4.4]nonane-8-carboxylate (0.16 g, 0.61 mmol, 1.10 equiv.), HATU (0.27 g, 0.71 mmol, 1.3 equiv.) and DIPEA (0.28 mL, 1.60 mmol, 3.0 equiv.). The reaction was stirred at room temperature for 30 minutes. Water (5 mL) was added to the reaction. The cloudy mixture was filtered, and the residue was dissolved in EtOAc, washed with brine, dried over Na₂SO₄, and concentrated to give compound 6 (0.23 g, 0.44 mmol, Yield 81.0%) as a yellow oil, which was used in the next step without further purification. LC/MS (ESI) m/z: 523 (M+H)⁺.

### Step 6: (S)-7-((4-(4-cyclopropylphenoxy)butanoyl)glycyl)-1,4-dioxa-7-azaspiro[4.4]nonane-8-carboxylic acid (7)

To a solution of compound 6 (0.2 g, 0.383 mmol, 1.0 equiv.) in EtOAc (3 mL) added 5% Pd/C (0.0815 g, 0.0383 mmol, 0.1 equiv.). The flask was evacuated and then backfilled with H₂ in a balloon. The reaction was stirred at room temperature overnight and then filtered through Celite pad. The filtrate was concentrated and dried in vacuo to give compound 7 (0.15 g, 0.347 mmol, Yield 90.62%) as a brown solid. LC/MS (ESI) m/z: 433 (M+H)⁺.

### Step 7: (S)-N-((4-carbamimidoylthiophen-2-yl)methyl)-7-((4-(4-cyclopropylphenoxy) butanoyl)glycyl)-1,4-dioxa-7-azaspiro[4.4]nonane-8-carboxamide (Compound 208)

To a solution of (8S)-7-[2-[4-(4-cyclopropylphenoxy)butanoylamino]acetyl]-1,4-dioxa-7-azaspiro[4.4]nonane-8-carboxylic acid (60 mg, 0.14 mmol, 1.0 equiv.) in DMF (2 mL) was added 5-(aminomethyl)thiophene-3-carboxamidine·HCl (29 mg, 0.15 mmol, 1.1 equiv.), HATU (63 mg, 0.17 mmol, 1.2 equiv.) and DIPEA (97 μL, 0.56 mmol, 4 equiv.). The reaction was stirred at room temperature for 10 minutes, then purified directly on HPLC to give Compound 208 (0.02 g, 0.17 mmol, Yield 30.0%). LC/MS (ESI) m/z: 570 (M+H)⁺. ¹H NMR (400 MHz, DMSO-d₆) δ 8.90 (s, 1H), 8.57 (t, J=5.9 Hz, 1H), 8.35 (d, J=5.7 Hz, 1H), 8.04 (d, J=6.2 Hz, 1H), 7.48 (d, J=13.1 Hz, 1H), 6.97 (d, J=8.2 Hz, 2H), 6.79 (d, J=8.3 Hz, 2H), 4.60 (d, J=8.4 Hz, 1H), 4.48 (d, J=5.7 Hz, 2H), 4.09-3.74 (m, 4H), 3.75-3.64 (m, 2H), 3.54 (d, J=10.9 Hz, 2H), 3.17 (m, 1H), 2.89 (d, J=17.6 Hz, 2H), 2.50 (m, 1H), 2.41-2.18 (m, 4H), 2.01 (dd, J=13.0, 6.7 Hz, 2H), 1.95-1.72 (m, 4H), 0.93-0.74 (m, 2H), 0.68-0.50 (m, 2H).

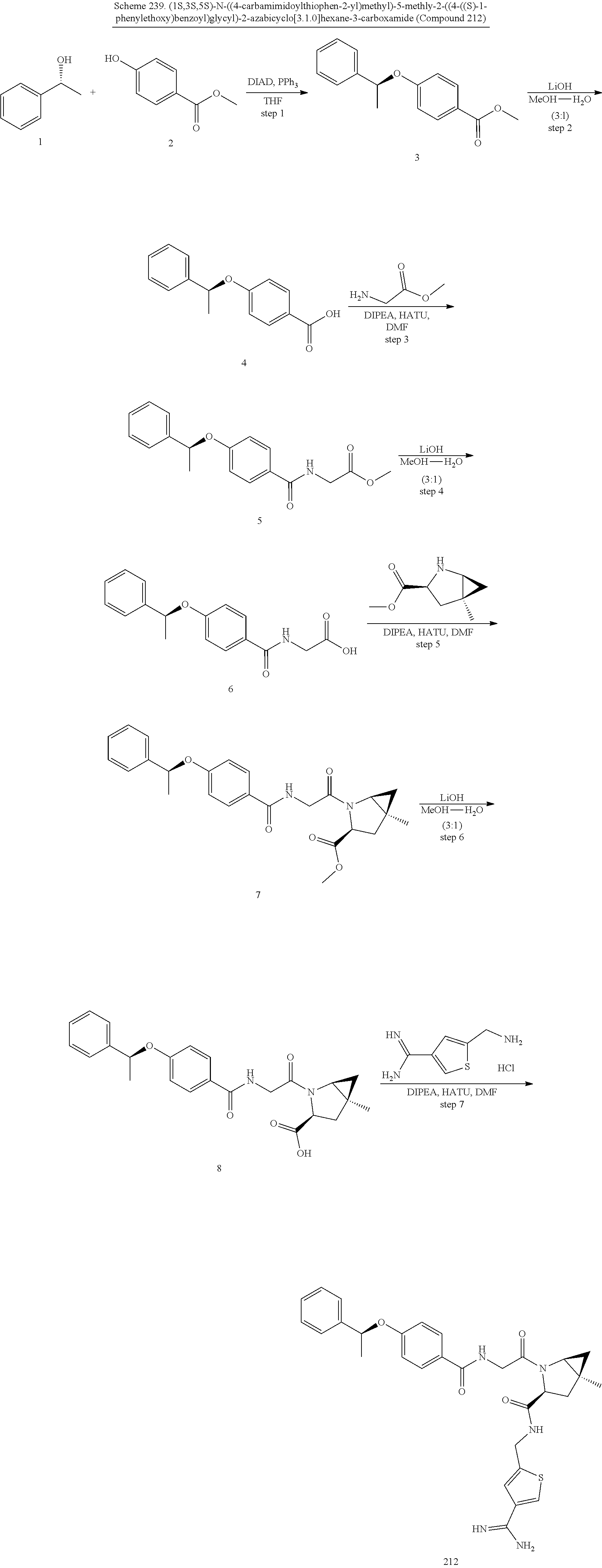

### Step 1: methyl (S)-4-(1-phenylethoxy)benzoate (3)

To a solution of (1R)-1-phenylethanol (0.5 g, 4.1 mmol, 1.0 equiv.) and methyl 4-hydroxybenzoate (0.69 g, 4.51 mmol, 1.1 equiv.) in THE (10 mL) at ice bath temperature was added PPh₃(2.26 g, 8.61 mmol, 2.1 equiv.) followed by diisopropyl azodicarboxylate (1.61 mL, 8.2 mmol, 2.0 equiv.). The reaction was stirred at room temperature for 16 hours, quenched with sat. NH₄Cl, and then diluted with EtOAc. The two layers were separated, and the aqueous layer was extracted with EtOAc (10 mL×2). The combined organic extracts were washed with brine, dried over MgSO₄ and concentrated. Purification by Combi-Flash; 12 g column, solvent A=Hexanes, solvent B=EtOAc, 100% A to 10% B gave compound 3 (0.6 g, 2.34 mmol, 57.16% Yield). LC/MS (ESI) m/z: 257 (M+H)⁺.

### Step 2: (S)-4-(1-phenylethoxy)benzoic acid (4)

To a solution of compound 3 (0.7 g, 2.73 mmol, 1.00 equiv.) in MeOH (12 mL) and water (4 mL) was added LiOH (0.2 g, 8.2 mmol, 3 equiv.). The reaction was stirred at room temperature overnight. The reaction was concentrated to remove MeOH and then diluted with EtOAc. The pH was adjusted to 1 using 1 N HCl. The two layers were separated, and the aqueous layer was extracted with EtOAc (10 mL×2). The combined organic extracts were washed with brine, dried over Na₂SO₄ and concentrated to obtain compound 4 (0.60 g, 2.48 mmol, 90.84% Yield) as a white solid. LC/MS (ESI) m/z: 243 (M+H)⁺.

### Step 3: methyl (S)-(4-(1-phenylethoxy)benzoyl)glycinate (5)

To a solution of compound 4 (0.5 g, 2.07 mmol, 1 equiv.) in DMF (5 mL) was added methyl 2-aminoacetate. HCl (0.31 g, 2.48 mmol, 1.2 equiv.), HATU (1.02 g, 2.69 mmol, 1.3 equiv.) and DIPEA (1.2 mL, 6.21 mmol, 3 equiv.). The reaction was stirred at room temperature for 1 hour. Water (10 mL) was added and the mixture stirred for 10 minutes. The product crashed out and the liquid was decanted, and the residue was dissolved in EtOAc, washed with brine, dried over MgSO₄ and concentrated to give compound 5 (0.5 g, 1.60 mmol, Yield 77.29%) as a white solid. LC/MS (ESI) m/z: 314 (M+H)⁺.

### Step 4: (S)-(4-(1-phenylethoxy)benzoyl)glycine (6)

To a solution of compound 5 (0.4 g, 1.28 mmol, 1.0 equiv.) in MeOH (6 mL) and water (2 mL) was added LiOH (0.1 g, 4.13 mmol, 3 equiv.). The reaction was stirred at room temperature overnight. The reaction was then concentrated to remove MeOH and diluted with EtOAc. The pH was adjusted to 1 using 1 N HCl. The two layers were separated, and the aqueous layer was extracted with EtOAc (10 mL×2). The combined organic extracts were washed with brine, dried over Na₂SO₄ and concentrated to give compound 6 (0.3 g, 1.0 mmol, 78.39% Yield) as a white solid. LC/MS (ESI) m/z: 300 (M+H)⁺.

### Step 5: methyl (1S,3S,5S)-5-methyl-2-((4-((S)-1-phenylethoxy)benzoyl) glycyl)-2-azabicyclo [3.1.0]hexane-3-carboxylate (7)

To a solution of compound 6 (0.08 g, 0.27 mmol, 1.0 equiv.) in DMF (2 mL) was added methyl (1S,3S,5S)-5-methyl-2-azabicyclo[3.1.0]hexane-3-carboxylate (0.05 g, 0.32 mmol, 1.2 equiv.), HATU (0.13 g, 0.35 mmol, 1.3 equiv.) and DIPEA (0.1 mL, 0.6 mmol, 3.0 equiv.). The reaction was stirred at room temperature for 30 minutes. Water (5 mL) and EtOAc (5 mL) were added. The two layers were separated, and the aqueous layer was extracted with EtOAc (5 mL×2), washed with brine, dried over Na₂SO₄, and concentrated. Purification by Combi Flash, 4 g column, solvent A=Hexanes, solvent B=EtOAc, 100% A to 25% B gave compound 7 (0.08 g, 0.18 mmol, Yield 67.96%) as a brown oil.

LC/MS (ESI) m/z: 437 (M+H)⁺.

### Step 6: (1S,3S,5S)-5-methyl-2-((4-((S)-1-phenylethoxy)benzoyl)glycyl)-2-azabicyclo[3.1.0]hexane-3-carboxylic acid (8)

To a solution of compound 7 (0.08 g, 0.18 mmol, 1.00 equiv.) in MeOH (3 mL) water (1 mL) was added LiOH (5 mg, 0.21 mmol, 1.1 equiv.). The reaction was stirred at room temperature overnight. The reaction was then concentrated to remove MeOH and diluted with EtOAc. The pH was adjusted to 1 using 1 N HCl. The two layers were separated, and the aqueous layer was extracted with EtOAc (5 mL×2). The combined organic extracts were washed with brine, dried over Na₂SO₄ and concentrated to give compound 8 (0.07 g, 0.2 mmol, 90% Yield) as a white solid. LC/MS (ESI) m/z: 423 (M+H)⁺.

### Step 7: (1S,3S,5S)-N-((4-carbamimidoylthiophen-2-yl)methyl)-5-methyl-2-((4-((S)-1-phenylethoxy)benzoyl)glycyl)-2-azabicyclo[3.1.0]hexane-3-carboxamide (Compound 212)

To a solution of compound 8 (0.07 g, 0.17 mmol, 1.0 equiv.) in DMF (2 mL) was added 5-(aminomethyl)thiophene-3-carboximidamide. HCl (0.035 g, 0.19 mmol, 1.1 equiv.), HATU (0.08 g, 0.20 mmol, 1.2 equiv.), and DIPEA (0.12 mL, 0.68 mmol, 4.0 equiv.). The reaction mixture was stirred at room temperature for 30 minutes and then purified directly by HPLC to give Compound 212 (0.02 g, 0.04 mmol, 24.1% Yield). LC/MS (ESI) m/z: 560 (M+H)⁺. ¹H NMR (400 MHz, DMSO-d₆) δ 8.50 (d, J=5.0 Hz, 1H), 8.41 (s, 1H), 8.33 (d, J=6.2 Hz, 1H), 7.72 (d, J=8.6 Hz, 1H), 7.47 (d, J=11.8 Hz, 1H), 7.41 (d, J=7.7 Hz, 1H), 7.34 (t, J=7.5 Hz, 1H), 7.25 (t, J=7.3 Hz, 1H), 6.96 (dd, J=9.0, 3.2 Hz, 1H), 5.60 (q, J=6.3 Hz, 1H), 4.84 (d, J=10.9 Hz, 1H), 4.65 (dd, J=11.3, 3.0 Hz, 1H), 4.53-4.18 (m, 2H), 4.02 (d, J=5.6 Hz, 1H), 3.98 (d, J=5.5 Hz, 1H), 3.59-3.46 (m, 1H), 3.40 (d, J=5.7 Hz, 1H), 2.42-2.17 (m, 1H), 1.99 (dd, J=13.3, 3.0 Hz, 1H), 1.57 (d, J=6.3 Hz, 3H), 1.21 (s, 3H), 1.13 (d, J=5.3 Hz, 1H), 0.63 (t, J=5.6 Hz, 1H).

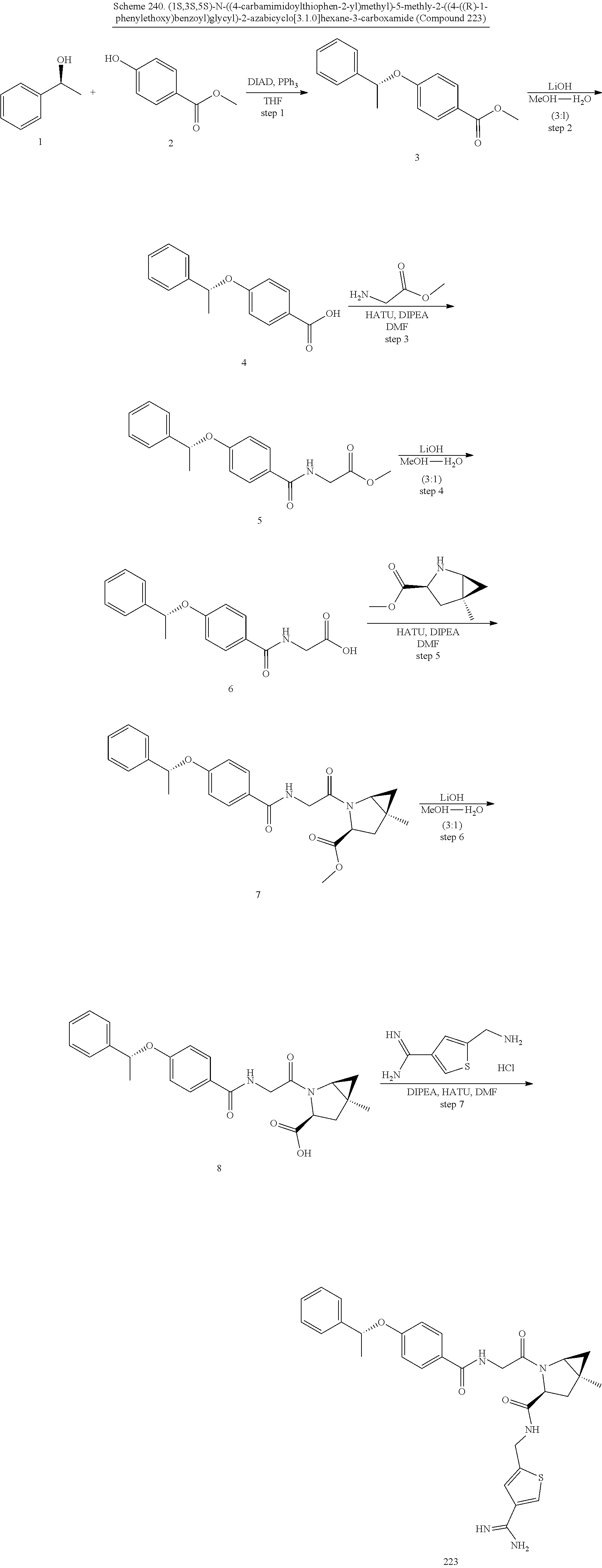

### Step 1: methyl (R)-4-(1-phenylethoxy)benzoate (3)

To a solution of (1 S)-1-phenylethanol (500 mg, 4.1 mmol, 1.0 equiv.) and methyl 4-hydroxybenzoate (0.69 g, 4.51 mmol, 1.1 equiv.) in TH (10 mL) at ice bath temperature was added PPh₃ (2.26 g, 8.61 mmol, 2.1 equiv.) followed by diisopropyl azodicarboxylate, DIAD, (1.65 mL, 8.2 mmol, 2.0 equiv.). The reaction was stirred at room temperature for 16 hours and quenched with sat. NH₄Cl and then diluted with EtOAc. The two layers were separated, and aqueous layer was extracted with EtOAc (10 mL×2). The combined organic extracts were washed with brine, dried over MgSO₄ and concentrated. Purification by Combi-Flash; 24 g column, solvent A=Hexanes, solvent B=EtOAc, 100% A to 10% B gave compound 3 (0.6 g, 2.34 mmol, 57.16% Yield). LC/MS (ESI) m/z: 257 (M+H)+.

### Step 2: (R)-4-(1-phenylethoxy)benzoic acid (4)

To a solution of compound 3 (0.5 g, 1.95 mmol, 1.0 equiv.) in MeOH (6 mL) water (2 mL) was added LiOH (0.14 g, 5.85 mmol, 3.0 equiv.). The reaction was stirred at room temperature overnight and concentrated to remove MeOH and then diluted with EtOAc. The pH was adjusted to 1 using 1 N HCl. The two layers were separated, and the aqueous layer was extracted with EtOAc (5 mL×2). The combined organic extracts were washed with brine, dried over Na₂SO₄ and concentrated to give compound 4 (0.42 g, 1.74 mmol, 89.0% Yield) as a white solid. LC/MS (ESI) m/z: 243 (M H).

### Step 3: methyl (R)-(4-(1-phenylethoxy)benzoyl)glycinate (5)

To a solution of compound 4 (0.4 g, 1.65 mmol, 1 equiv.) in DMF (2 mL) was added glycine methyl ester hydrochloride (0.25 g, 1.98 mmol, 1.2 equiv.), HATU (0.94 g, 2.48 mmol, 1.5 equiv.) and DIPEA (0.86 mL, 4.95 mmol, 3 equiv.). The reaction was stirred at room temperature for 1 hour. Water (10 mL) was added and the solid collected by filtration and then dried to give compound 5 (0.49 g, 1.56 mmol, Yield 94.55%) as a light brown solid. LC/MS (ESI) m/z: 314 (M+H)⁺.

### Step 4: (R)-(4-(1-phenylethoxy)benzoyl)glycine (6)

To a solution of compound 5 (0.4 g, 1.28 mmol, 1.0 equiv.) in MeOH (6 mL) water (2 mL) was added LiOH (0.1 g, 3.83 mmol, 3 equiv.). The reaction was stirred at room temperature overnight. The reaction was then concentrated to remove MeOH and diluted with EtOAc. The pH was adjusted to 1 using 1 N HCl. The two layers were separated, and the aqueous layer was extracted with EtOAc (5 mL×2). The combined organic extracts were washed with brine, dried over Na₂SO₄ and concentrated to give compound 6 (0.31 g, 1.04 mmol, Yield 81.0%) as a white solid. LC/MS (ESI) m/z: 300 (M+H)⁺.

### Step 5: methyl (1S,3S,5S)-5-methyl-2-((4-((R)-1-phenylethoxy)benzoyl) glycyl)-2-azabicyclo [3.1.0]hexane-3-carboxylate (7)

To a solution of compound 6 (0.22 g, 0.74 mmol, 1.0 equiv.) in DMF (2 mL) was added methyl (1S,3S,5S)-5-methyl-2-azabicyclo[3.1.0]hexane-3-carboxylate (0.14 g, 0.90 mmol, 1.2 equiv.), HATU (0.36 g, 0.95 mmol, 1.3 equiv.) and DIPEA (0.38 mL, 2.18 mmol, 3.0 equiv.). The reaction was stirred at room temperature for 30 minutes. Water (5 mL) and the liquid decanted. The residue was taken up in EtOAc washed with brine, dried over Na₂SO₄, and concentrated. Purification by Combi Flash; 12 g column, solvent A=Hexanes, solvent B=EtOAc, 100% A to 25% B gave compound 7 (0.26 g, 0.6 mmol, Yield 81.08%) as a brown oil. LC/MS (ESI) m/z: 437 (M+H)⁺.

### Step 6: (1S,3S,5S)-5-methyl-2-((4-((R)-1-phenylethoxy)benzoyl)glycyl)-2-azabicyclo[3.1.0]hexane-3-carboxylic acid (8)

To a solution of compound 7 (0.2 g, 0.46 mmol, 1.0 equiv.) in MeOH (3 mL) water (1 mL) was added LiOH (0.012 g, 0.5 mmol, 1.1 equiv.). The reaction was stirred at room temperature overnight. The reaction was then concentrated to remove MeOH and then diluted with EtOAc. The pH was adjusted to 1 using 1 N HCl. The two layers were separated, and the aqueous layer was extracted with EtOAc (5 mL×2). The combined organic extracts were washed with brine, dried over Na₂SO₄ and concentrated to give compound 8 (0.15 g, 0.36 mmol, 78.26% Yield) as a yellow sticky solid. LC/MS (ESI) m/z: 423 (M+H)⁺.

### Step 7: (1S,3S,5S)-N-((4-carbamimidoylthiophen-2-yl)methyl)-5-methyl-2-((4-((R)-1-phenylethoxy)benzoyl)glycyl)-2-azabicyclo[3.1.0]hexane-3-carboxamide (Compound 223)

To a solution of compound 8 (0.15 g, 0.36 mmol, 1.0 equiv.) in DMF (2 mL) was added 5-(aminomethyl)thiophene-3-carboximidamide. HCl (0.08 g, 0.39 mmol, 1.1 equiv.), HATU (0.16 g, 0.42 mmol, 1.1 equiv.), and DIPEA (0.2 mL, 1.09 mmol, 3.0 equiv.). The reaction was stirred at room temperature for 30 minutes and then purified directly by HPLC to give Compound 223 (15 mg, 0.03 mmol, 7.5% Yield). LC/MS (ESI) m/z: 560 (M+H)⁺. ¹H NMR (400 MHz, DMSO-d₆) δ 8.93 (s, 1H), 8.51 (q, J=5.8 Hz, 1H), 8.32 (s, 1H), 7.73 (dd, J=9.0, 2.5 Hz, 2H), 7.58-7.15 (m, 4H), 6.96 (dd, J=8.9, 3.1 Hz, 2H), 5.60 (q, J=6.3 Hz, 1H), 4.70-4.52 (m, 1H), 4.50-4.18 (m, 2H), 4.09-3.92 (m, 1H), 2.90 (d, J=8.5 Hz, 1H), 2.50 (s, 3H), 2.28 (q, J=11.9 Hz, 1H), 1.57 (d, J=6.3 Hz, 3H), 1.20 (d, J=8.8 Hz, 2H), 1.15-1.08 (s, 3H), 0.90 (d, J=5.7 Hz, 1H), 0.65 (t, J=5.7 Hz, 1H).

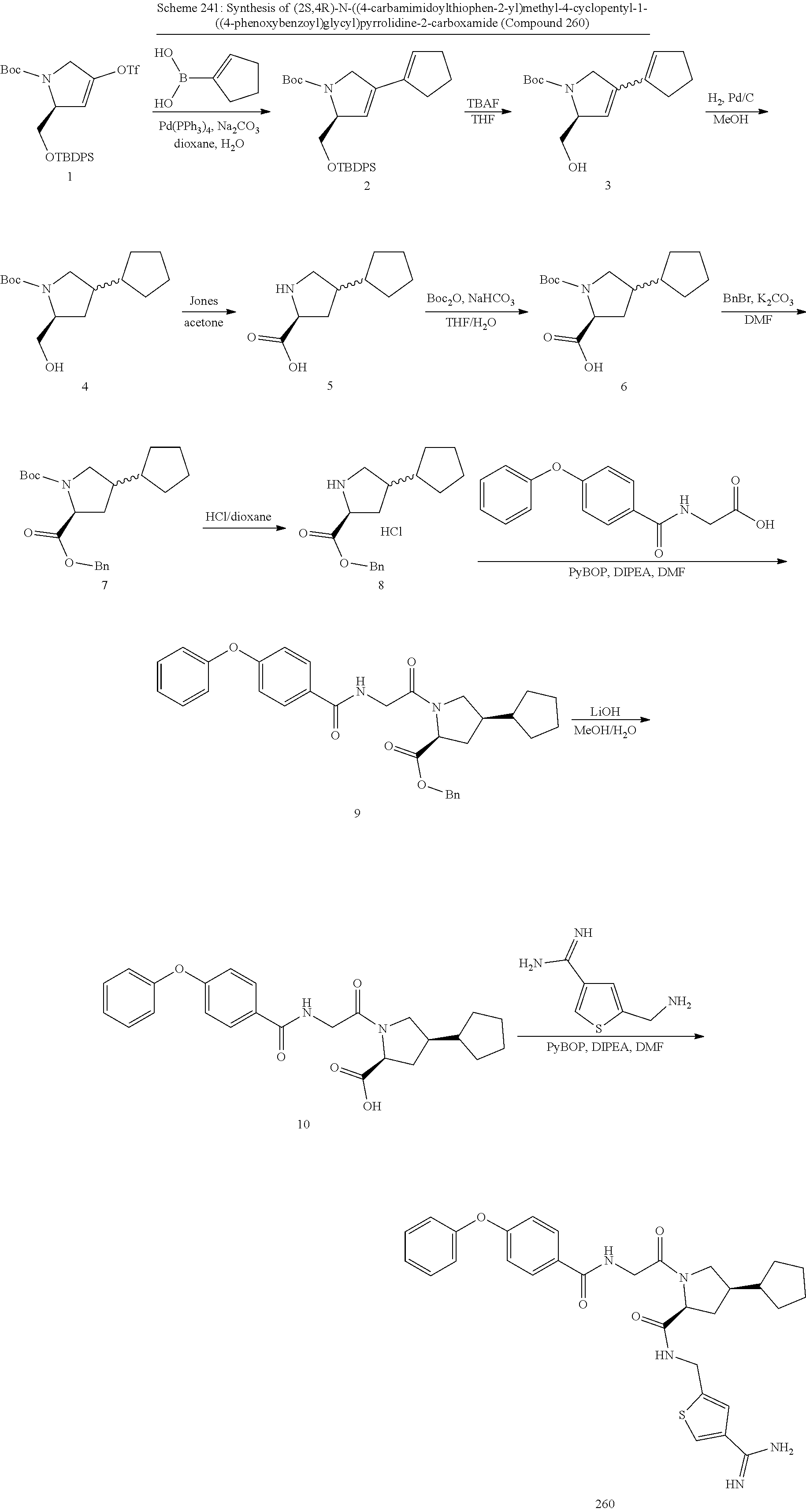

### Step 1: tert-butyl (S)-2-(((tert-butyldiphenylsilyl)oxy)methyl)-4-(cyclopent-1-en-1-yl)-2,5-dihydro-1H-pyrrole-1-carboxylate

To a mixture of tert-butyl (S)-2-(((tert-butyldiphenylsilyl)oxy)methyl)-4-(((trifluoromethyl) sulfonyl)oxy)-2,5-dihydro-1H-pyrrole-1-carboxylate (1.5 g, 2.6 mmol) and cyclopent-1-en-1-ylboronic acid (290 mg, 2.6 mmol) in 1,4-dioxane (10 mL) and water (5 mL) was added Pd(PPh₃)₄(210 mg, 0.18 mmol) and Na₂CO₃ (827 mg, 7.8 mmol). The mixture was degassed under N₂ atmosphere for three times and stirred at 85° C. for 2 hours. The mixture was diluted with water and extracted with EtOAc twice. The combined organic layers were washed with brine, dried over anhydrous Na₂SO₄, filtered and concentrated to dryness under reduced pressure. The residue was purified by column chromatography (SiO₂, 0-2% EtOAc in PE) to give the tert-butyl (S)-2-(((tert-butyldiphenylsilyl)oxy)methyl)-4-(cyclopent-1-en-1-yl)-2,5-dihydro-1H-pyrrole-1-carboxylate (1.2 g, yield 91.5%) as yellow oil. LCMS (ESI) m/z: 504 (M+H)⁺.

### Step 2: tert-butyl (2S)-2-(((tert-butyldiphenylsilyl)oxy)methyl)-4-cyclopentylpyrrolidine-1-carboxylate

To a solution of tert-butyl (S)-2-(((tert-butyldiphenylsilyl)oxy)methyl)-4-(cyclopent-1-en-1-yl)-2,5-dihydro-1H-pyrrole-1-carboxylate (1.2 g, 2.4 mmol) in THE (10 mL) was added TBAF (4.7 mL, 4.7 mmol) at 0° C. and the mixture was stirred under N₂ atmosphere at room temperature for 1 hour. The mixture was diluted with water and extracted with EtOAc twice. The combined organic layers were washed with brine, dried over anhydrous Na₂SO₄, filtered and concentrated to dryness under reduced pressure. The residue was purified by flash chromatography (silica gel, 0-30% EtOAc in PE) to give tert-butyl (S)-4-(cyclopent-1-en-1-yl)-2-(hydroxymethyl)-2,5-dihydro-1H-pyrrole-1-carboxylate (530 mg, yield 83.4%) as a yellow oil. LCMS (ESI) m/z: 268 (M+H)⁺.

### Step 3: tert-butyl (2S)-4-cyclopentyl-2-(hydroxymethyl)pyrrolidine-1-carboxylate

To a solution of tert-butyl (S)-4-(cyclopent-1-en-1-yl)-2-(hydroxymethyl)-2,5-dihydro-1H-pyrrole-1-carboxylate (530 mg, 2.0 mmol) in MeOH (7 mL) was added Pd/C (140 mg, 10% wt.), the mixture was degassed under N₂ atmosphere for three times and stirred under a H₂ balloon at room temperature overnight. The mixture was filtered and the filtrate was concentrated under reduced pressure to dryness to give tert-butyl (2S)-4-cyclopentyl-2-(hydroxymethyl)pyrrolidine-1-carboxylate (450 mg, yield 83.4%) as a colorless oil, which was used directly in the next step. LC/MS (ESI) (m/z): 270 (M+H)⁺.

### Step 4: (2S)-4-cyclopentylpyrrolidine-2-carboxylic acid

To a mixture of tert-butyl (2S)-4-cyclopentyl-2-(hydroxymethyl)pyrrolidine-1-carboxylate (450 mg, 2.0 mmol) in acetone (10 mL) was added Jones oxidant (0.9 mL, newly prepared) at 0° C. and the mixture was stirred at room temperature for 20 minutes. The mixture was quenched with saturated aq.Na₂S₂O₃ solution and extracted with EtOAc twice. The combined organic layers were washed with brine, dried over anhydrous Na₂SO₄, filtered and concentrated to dryness under reduced pressure to give the (2S)-4-cyclopentylpyrrolidine-2-carboxylic acid (500 mg, crude) as a yellow oil, which was used directly in the next step without further purification. LCMS (ESI) m/z: 284 (M+H)⁺.

### Step 5: (2S)-1-(tert-butoxycarbonyl)-4-cyclopentylpyrrolidine-2-carboxylic acid

To a solution of (2S)-4-cyclopentylpyrrolidine-2-carboxylic acid (500 mg, crude) THF (10 mL) and water (2 mL) was added Boc₂O (1.4 g, 6.6 mmol) and NaHCO₃ (550 mg, 6.6 mmol) and the mixture was stirred at room temperature overnight. The mixture was diluted with water and extracted with EtOAc twice. The combined organic layers were washed with brine, dried over anhydrous Na₂SO₄, filtered and concentrated under reduced pressure to dryness. The residue was purified by flash chromatography (silica gel, 0-7% MeOH in DCM) to give (2S)-1-(tert-butoxycarbonyl)-4-cyclopentylpyrrolidine-2-carboxylic acid (400 mg, yield 84.6% via two steps) as a colorless oil. LCMS (ESI) m/z: 284 (M+H)⁺.

### Step 6: 2-benzyl 1-(tert-butyl) (2S)-4-cyclopentylpyrrolidine-1,2-dicarboxylate

To a solution of (2S)-1-(tert-butoxycarbonyl)-4-cyclopentylpyrrolidine-2-carboxylic acid (400 mg, 1.7 mmol) in DMF (4 mL) was added Cs₂CO₃ (1.6 g, 5.1 mmol) and BnBr (870 mg, 5.1 mmol) and the reaction mixture was stirred at room temperature overnight. The mixture was diluted with water and extracted with EtOAc twice. The combined organic layers were washed with brine, dried over anhydrous Na₂SO₄, filtered and concentrated to dryness under reduced pressure. The residue was purified by column chromatography on (silica gel, 0-30% EtOAc in PE) to give 2-benzyl 1-(tert-butyl) (2S)-4-cyclopentylpyrrolidine-1,2-dicarboxylate (330 mg, yield 51.8%) as a yellow oil. LCMS (ESI) m/z: 374 (M+H)⁺.

### Step 7: benzyl (2S)-4-cyclopentylpyrrolidine-2-carboxylate hydrochloride

A solution of 2-benzyl 1-(tert-butyl) (2S)-4-cyclopentylpyrrolidine-1,2-dicarboxylate (100 mg, 0.27 mmol) in HCl/1,4-dioxane (2 mL) was stirred under N₂ atmosphere at room temperature for 30 minutes. The reaction mixture was concentrated to dryness under reduced pressure, co-evaporated with DCM twice and dried under vacuum to give benzyl (2S)-4-cyclopentylpyrrolidine-2-carboxylate hydrochloride (70 mg, yield 95.0%) as a yellow solid, which was used directly in the next step without further purification. LC/MS (ESI) m/z: 274 (M+H)⁺.

### Step 8: benzyl (2S,4S)-4-cyclopentyl-1-((4-phenoxybenzoyl)glycyl)pyrrolidine-2-carboxylate

To a mixture of benzyl (2S)-4-cyclopentylpyrrolidine-2-carboxylate hydrochloride (70 mg, 0.26 mmol) and (4-phenoxybenzoyl)glycine (70 mg, 0.26 mmol) in DMF (1 mL) was added DIPEA (168 mg, 1.3 mmol) and PyBOP (135 mg, 0.26 mmol) at 0° C. under N₂ atmosphere and the mixture was stirred at 25° C. for 1 hour. The mixture was diluted with EtOAc, washed with water and brine, dried over anhydrous Na₂SO₄, filtered and concentrated under reduced pressure to dryness. The residue was purified by flash chromatography (silica gel, 0-30% EtOAc in PE) and further purified prep.HPLC to give benzyl (2S,4S)-4-cyclopentyl-1-((4-phenoxybenzoyl)glycyl)pyrrolidine-2-carboxylate (92 mg, yield 67.2%) as a yellow solid. LC/MS (ESI) m/z: 527 (M+H)⁺.

### Step 9: (2S,4R)-4-cyclopentyl-1-((4-phenoxybenzoyl)glycyl)pyrrolidine-2-carboxylic acid

To a solution of benzyl (2S,4S)-4-cyclopentyl-1-((4-phenoxybenzoyl)glycyl)pyrrolidine-2-carboxylate (92 mg, 0.17 mmol) in MeOH (1.5 mL) and water (1.0 mL) was added LiOH·H₂O (42 mg, 1.0 mmol) at 0° C. and the mixture was stirred at room temperature for 1 hour. The mixture was acidified with 1N aq.HCl to pH-3 and extracted with EtOAc twice. The combined organic layers were washed with brine, dried over anhydrous Na₂SO₄, filtered and concentrated under reduced pressure to dryness to give (2S,4R)-4-cyclopentyl-1-((4-phenoxybenzoyl)glycyl)pyrrolidine-2-carboxylic acid (70 mg, yield 94.4%) as a colorless oil, which was used directly in the next reaction. LCMS (ESI) (m/z): 437 (M+H)⁺.

### Step 10: (2S,4R)-N-((4-carbamimidoylthiohen-2-yl)methyl)-4-cyclopentyl-1-((4-phenoxybenzoyl)glycyl)pyrrolidine-2-carboxamide (Compound 260)

To a mixture of (2S,4R)-4-cyclopentyl-1-((4-phenoxybenzoyl)glycyl)pyrrolidine-2-carboxylic acid (40 mg, 0.09 mmol) and 5-(aminomethyl)thiophene-3-carboximidamide (27 mg, 0.17 mmol) in DMF (0.7 mL) was added DIPEA (67 mg, 0.52 mmol) and PyBOP (50 mg, 0.096 mmol) at 0° C. and the mixture was stirred at room temperature for 1 hour. The organic layer was diluted with water and extracted with EtOAc twice. The combined organic layers were washed with brine, dried over anhydrous Na₂SO₄, filtered and concentrated to dryness under reduced pressure. The residue was purified by flash chromatography (silica gel, 0-10% MeOH in DCM) to give Compound 260 (6 mg, yield 12.0%) as a white solid. ¹H NMR (400 MHz, CD₃OD) δ 8.09 (s, 1H), 7.84 (d, J=8.6 Hz, 2H), 7.41 (t, J=7.9 Hz, 3H), 7.20 (t, J=7.4 Hz, 1H), 7.06 (d, J=7.9 Hz, 2H), 7.00 (d, J=8.7 Hz, 2H), 4.62 (d, J=14.8 Hz, 1H), 4.56 (s, 2H), 4.39 (d, J=8.8 Hz, 1H), 4.26 (d, J=16.5 Hz, 1H), 4.14 (d, J=16.5 Hz, 1H), 3.97-3.91 (m, 1H), 2.43 (dd, J=12.3, 6.9 Hz, 1H), 2.13 (dd, J=43.0, 19.6 Hz, 3H), 1.78 (s, 3H), 1.67-1.57 (m, 4H), 1.25-1.22 (m, 1H). LC/MS (ESI) (m/z): 574 (M+H)⁺.

### Example 2. Non-Limiting Examples of Compounds of the Present Disclosure

Table 1 shows illustrative complement pathway with characterizing data. The assay of Example 3 was used to determine the IC₅₀'s of the compounds. Other standard complement assays are also available. Three ***s are used to denote compounds with an IC₅₀ less than 100 nanomolar; two ** indicates a compound with an IC₅₀ greater than 100 nanomolar and less than 1 micromolar, and one* denotes compounds with an IC₅₀ greater than 1 micromolar.

TABLE 1
Non-limiting Examples of Compounds of the Present Disclosure

| | | | | RT | MS |
|---|---|---|---|---|---|
| | | | C1s | min | (M |
| | | | Protease | (Method | + |
| # | Structure | Name | Activity | A) | 1) |
| 1 | 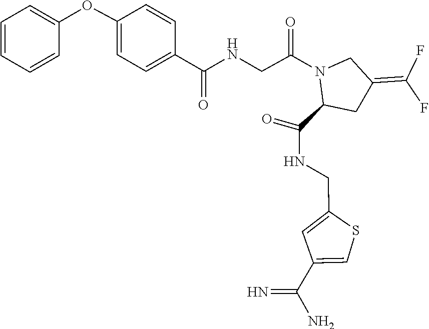 [SMILES: N=C(N)c1csc(CNC(=O)[C@@H]2CC(=C(F)F)CN2C(=O)CNC(=O)c2ccc(Oc3ccccc3)cc2)c1] | (S)-N-((4- carbamimidoylthiophen- 2-yl)methyl)-4- (difluoromethylene)-1- ((4- phenoxybenzoyl)glycyl) pyrrolidine-2- carboxamide | *** | 1.26 | 554 |
| 2 | 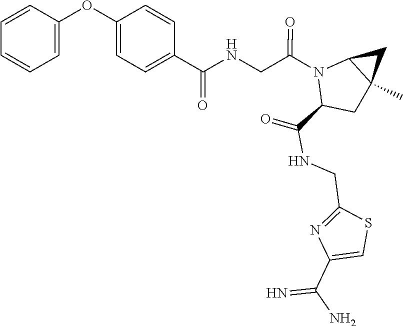 [SMILES: C[C@@]12C[C@@H]1N(C(=O)CNC(=O)c1ccc(Oc3ccccc3)cc1)[C@H](C(=O)NCc1nc(C(=N)N)cs1)C2] | (1S,3S,5S)-N-((4- carbamimidoylthiazol- 2-yl)methyl)-5-methyl- 2-((4- phenoxybenzoyl)glycyl)- 2- azabicyclo[3.1.0]hexane- 3-carboxamide | *** | 1.28 | 533 |
| 3 | 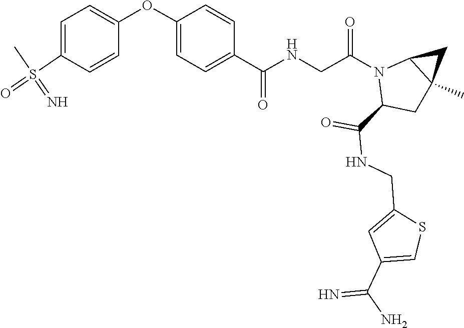 [SMILES: C[C@@]12C[C@@H]1N(C(=O)CNC(=O)c1ccc(Oc3ccc(S(C)(=N)=O)cc3)cc1)[C@H](C(=O)NCc1cc(C(=N)N)cs1)C2] | (1S,3S,5S)-N-((4- carbamimidoylthiophen- 2-yl)methyl)-5-methyl- 2-((4-(4-(S- methylsulfonimidoyl) phenoxy)benzoyl)glycyl)- 2-azabicyclo[3.1.0] hexane- 3-carboxamide | *** | 0.79 | 609 |
| 4 | 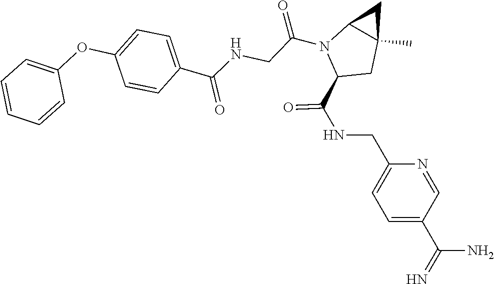 [SMILES: C[C@@]12C[C@@H]1N(C(=O)CNC(=O)c1ccc(Oc3ccccc3)cc1)[C@H](C(=O)NCc1ccc(C(=N)N)cn1)C2] | (1S,3S,5S)-N-((5- carbamimidoylpyridin- 2-yl)methyl)-5-methyl- 2-((4- phenoxybenzoyl)glycyl)- 2- azabicyclo[3.1.0]hexane- 3-carboxamide | ** | 1.23 | 527 |
| 5 | 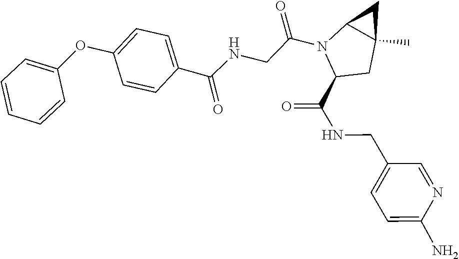 [SMILES: C[C@@]12C[C@@H]1N(C(=O)CNC(=O)c1ccc(Oc3ccccc3)cc1)[C@H](C(=O)NCc1ccc(N)nc1)C2] | (1S,3S,5S)-N-((6- aminopyridin-3- yl)methyl)-5-methyl-2- ((4- phenoxybenzoyl)glycyl)- 2- azabicyclo[3.1.0]hexane- 3-carboxamide | ** | 1.28 | 500 |
| 6 | 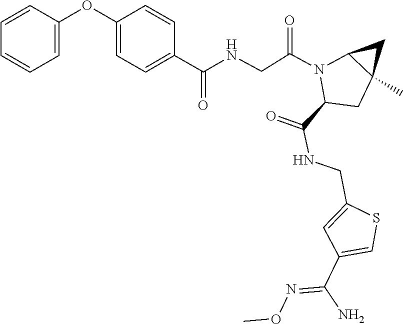 [SMILES: CO/N=C(\N)c1csc(CNC(=O)[C@@H]2C[C@]3(C)C[C@@H]3N2C(=O)CNC(=O)c2ccc(Oc3ccccc3)cc2)c1] | (1S,3S,5S)-N-((4-((Z)- N′- methoxycarbamimidoyl) thiophen-2-yl)methyl)- 5-methyl-2-((4- phenoxybenzoyl)glycyl)- 2- azabicyclo[3.1.0]hexane- 3-carboxamide | * | 1.78 | 562 |
| 7 | 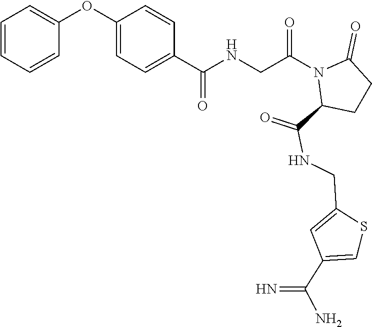 [SMILES: N=C(N)c1csc(CNC(=O)[C@@H]2CCC(=O)N2C(=O)CNC(=O)c2ccc(Oc3ccccc3)cc2)c1] | (S)-N-((4- carbamimidoylthiophen- 2-yl)methyl)-5-oxo-1- ((4- phenoxybenzoyl)glycyl) pyrrolidine-2- carboxamide | *** | 1.13 | 520 |
| 8 | 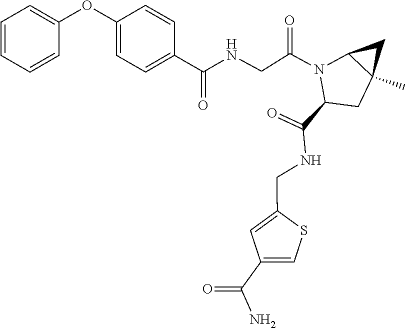 [SMILES: C[C@@]12C[C@@H]1N(C(=O)CNC(=O)c1ccc(Oc3ccccc3)cc1)[C@H](C(=O)NCc1cc(C(N)=O)cs1)C2] | (1S,3S,5S)-N-((4- carbamoylthiophen-2- yl)methyl)-5-methyl-2- ((4- phenoxybenzoyl)glycyl)- 2- azabicyclo[3.1.0]hexane- 3-carboxamide | * | 1.70 | 533 |
| 9 | 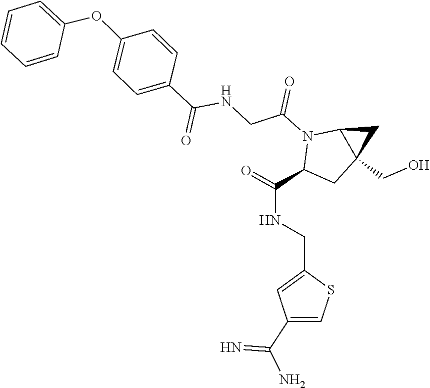 [SMILES: N=C(N)c1csc(CNC(=O)[C@@H]2C[C@]3(CO)C[C@@H]3N2C(=O)CNC(=O)c2ccc(Oc3ccccc3)cc2)c1] | (1S,3S,5R)-N-((4- carbamimidoylthiophen- 2-yl)methyl)-5- (hydroxymethyl)-2-((4- phenoxybenzoyl)glycyl)- 2- azabicyclo[3.1.0]hexane- 3-carboxamide | *** | 1.03 | 548 |
| 10 | 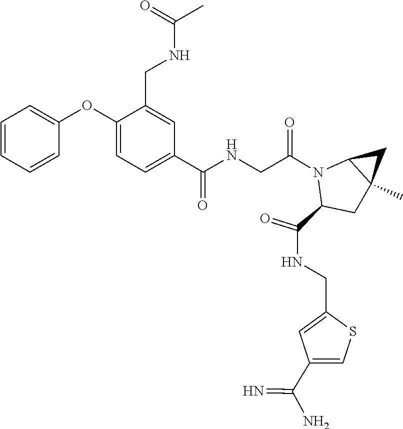 [SMILES: CC(=O)NCc1cc(C(=O)NCC(=O)N2[C@H]3C[C@@]3(C)C[C@H]2C(=O)NCc2cc(C(=N)N)cs2)ccc1Oc1ccccc1] | (1S,3S,5S)-2-((3- (acetamidomethyl)-4- phenoxybenzoyl)glycyl)- N-((4- carbamimidoylthiophen- 2-yl)methyl)-5-methyl- 2- azabicyclo[3.1.0]hexane- 3-carboxamide | *** | 1.04 | 603 |
| 11 | 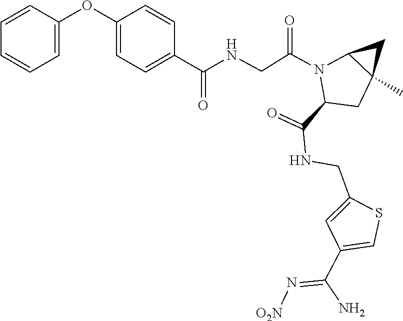 [SMILES: C[C@@]12C[C@@H]1N(C(=O)CNC(=O)c1ccc(Oc3ccccc3)cc1)[C@H](C(=O)NCc1cc(/C(N)=N/[N+](=O)[O-])cs1)C2] | (1S,3S,5S)-5-methyl-N- ((4-((Z)-N′- nitrocarbamimidoyl)thio phen-2-yl)methyl)-2- ((4- phenoxybenzoyl)glycyl)- 2- azabicyclo[3.1.0]hexane- 3-carboxamide | * | 1.36 | 577 |
| 12 | 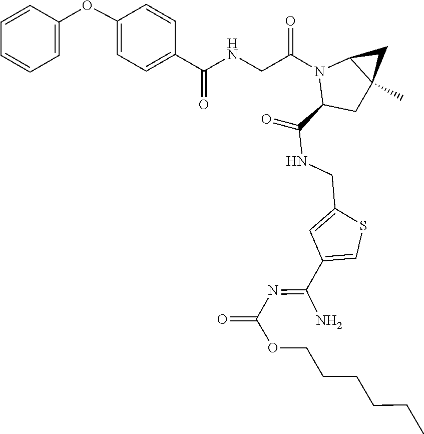 [SMILES: CCCCCCOC(=O)/N=C(\N)c1csc(CNC(=O)[C@@H]2C[C@]3(C)C[C@@H]3N2C(=O)CNC(=O)c2ccc(Oc3ccccc3)cc2)c1] | (Z)-hexyl (amino(5- (((1S,3S,5S)-5-methyl- 2-((4- phenoxybenzoyl)glycyl)- 2- azabicyclo[3.1.0] hexane-3- carboxamido)methyl) thiophen-3- yl)methylene)carbamate | * | 1.90 | 660 |
| 13 | 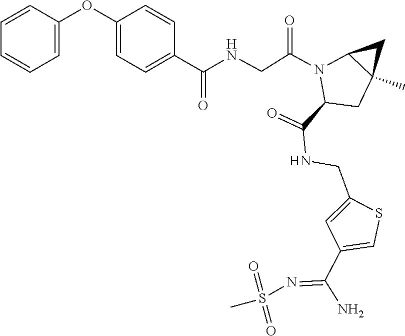 [SMILES: C[C@@]12C[C@@H]1N(C(=O)CNC(=O)c1ccc(Oc3ccccc3)cc1)[C@H](C(=O)NCc1cc(/C(N)=N/S(C)(=O)=O)cs1)C2] | (1S,3S,5S)-5-methyl-N- ((4-((Z)-N′- (methylsulfonyl)carbam imidoyl)thiophen-2- yl)methyl)-2-((4- phenoxybenzoyl)glycyl)- 2-azabicyclo[3.1.0] hexane-3-carboxamide | * | 1.81 | 610 |
| 14 | 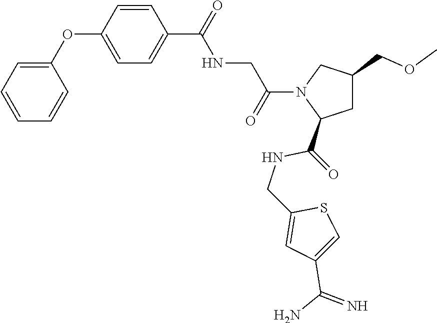 [SMILES: COC[C@H]1C[C@@H](C(=O)NCc2cc(C(=N)N)cs2)N(C(=O)CNC(=O)c2ccc(Oc3ccccc3)cc2)C1] | (2S,4S)-N-((4- carbamimidoylthiophen- 2-yl)methyl)-4- (methoxymethyl)-1-((4- phenoxybenzoyl)glycyl) pyrrolidine-2- carboxamide | *** | 1.11 | 550 |
| 15 | 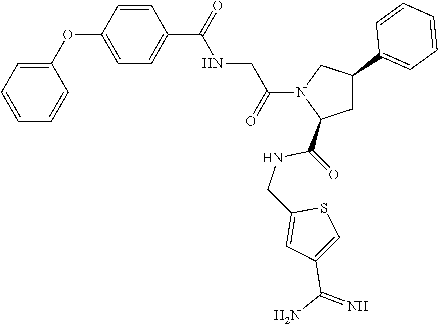 [SMILES: N=C(N)c1csc(CNC(=O)[C@@H]2C[C@H](c3ccccc3)CN2C(=O)CNC(=O)c2ccc(Oc3ccccc3)cc2)c1] | (2S,4R)-N-((4- carbamimidoylthiophen- 2-yl)methyl)-1-((4- phenoxybenzoyl)glycyl)- 4-phenylpyrrolidine-2- carboxamide | ** | 1.55 | 582 |
| 16 | 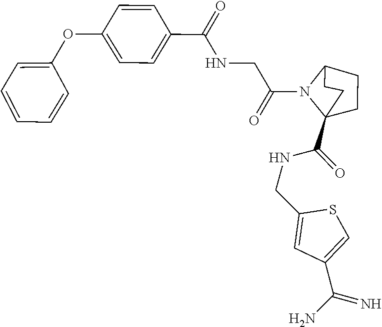 | N-((4- carbamimidoylthiophen- 2-yl)methyl)-7-((4- phenoxybenzoyl)glycyl)- 7- azabicyclo[2.2.1]heptane- 1-carboxamide | ** | 1.19 | 532 |
| 17 | 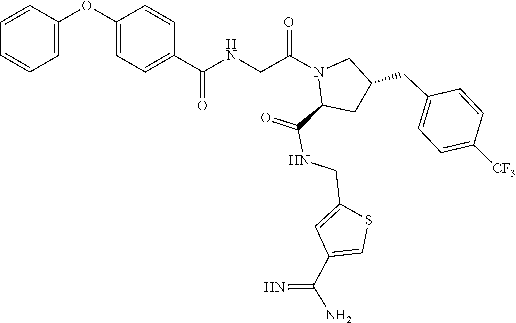 [SMILES: N=C(N)c1csc(CNC(=O)[C@@H]2C[C@@H](Cc3ccc(C(F)(F)F)cc3)CN2C(=O)CNC(=O)c2ccc(Oc3ccccc3)cc2)c1] | (2S,4R)-N-((4- carbamimidoylthiophen- 2-yl)methyl)-1-((4- phenoxybenzoyl)glycyl)- 4-(4- (trifluoromethyl)benzyl) pyrrolidine-2- carboxamide | *** | 1.75 | 664 |
| 18 | 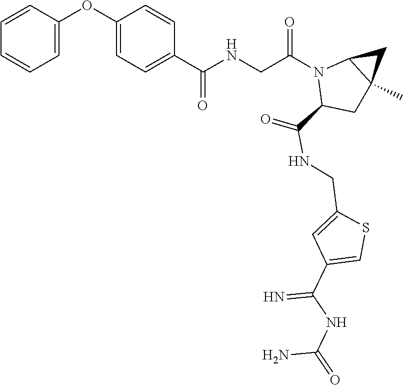 [SMILES: C[C@@]12C[C@@H]1N(C(=O)CNC(=O)c1ccc(Oc3ccccc3)cc1)[C@H](C(=O)NCc1cc(C(=N)NC(N)=O)cs1)C2] | (1S,3S,5S)-N-((4-(N- carbamoylcarbamimido yl) thiophen-2- yl)methyl)-5-methyl-2- ((4- phenoxybenzoyl)glycyl)- 2-azabicyclo[3.1.0] hexane-3-carboxamide | *** | 1.24 | 575 |
| 19 | 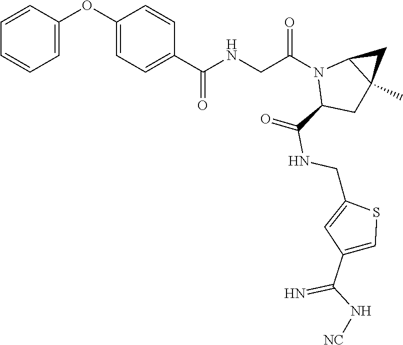 [SMILES: C[C@@]12C[C@@H]1N(C(=O)CNC(=O)c1ccc(Oc3ccccc3)cc1)[C@H](C(=O)NCc1cc(C(=N)NC#N)cs1)C2] | (1S,3S,5S)-N-((4-(N- cyanocarbamimidoyl)th iophen-2-yl)methyl)-5- methyl-2-((4- phenoxybenzoyl)glycyl)- 2- azabicyclo[3.1.0]hexane- 3-carboxamide | * | 1.84 | 557 |
| 20 | 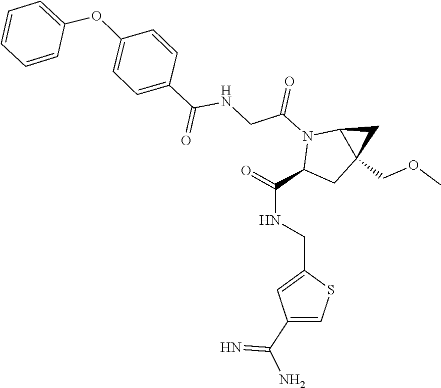 [SMILES: COC[C@@]12C[C@@H]1N(C(=O)CNC(=O)c1ccc(Oc3ccccc3)cc1)[C@H](C(=O)NCc1cc(C(=N)N)cs1)C2] | (1S,3S,5R)-N-((4- carbamimidoylthiophen- 2-yl)methyl)-5- (methoxymethyl)-2-((4- phenoxybenzoyl)glycyl)- 2- azabicyclo[3.1.0]hexane- 3-carboxamide | *** | 1.17 | 562 |
| 21 | 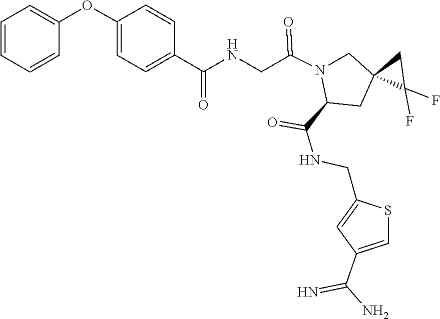 [SMILES: N=C(N)c1csc(CNC(=O)[C@@H]2C[C@@]3(CN2C(=O)CNC(=O)c2ccc(Oc4ccccc4)cc2)CC3(F)F)c1] | (3S,6S)-N-((4- carbamimidoylthiophen- 2-yl)methyl)-1,1- difluoro-5-((4- phenoxybenzoyl)glycyl)- 5- azaspiro[2.4]heptane- 6-carboxamide | *** | 1.29 | 568 |
| 22 | 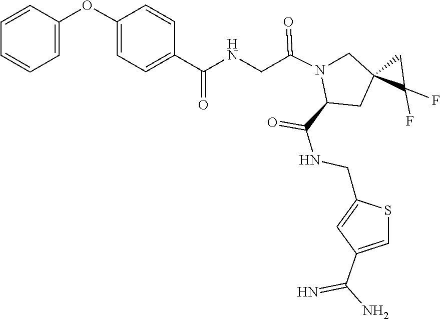 [SMILES: N=C(N)c1csc(CNC(=O)[C@@H]2C[C@]3(CN2C(=O)CNC(=O)c2ccc(Oc4ccccc4)cc2)CC3(F)F)c1] | (3R,6S)-N-((4- carbamimidoylthiophen- 2-yl)methyl)-1,1- difluoro-5-((4- phenoxybenzoyl)glycyl)- 5- azaspiro[2.4]heptane- 6-carboxamide | *** | 1.20 | 568 |
| 23 | 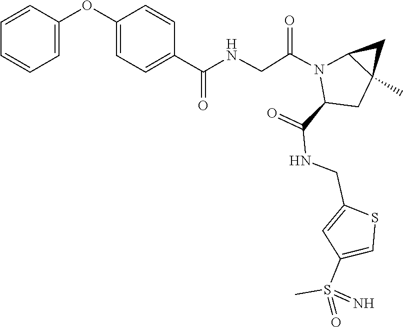 [SMILES: C[C@@]12C[C@@H]1N(C(=O)CNC(=O)c1ccc(Oc3ccccc3)cc1)[C@H](C(=O)NCc1cc(S(C)(=N)=O)cs1)C2] | (1S,3S,5S)-5-methyl-N- ((4-(S- methylsulfonimidoyl) thiophen-2-yl)methyl)-2- ((4- phenoxybenzoyl)glycyl)- 2- azabicyclo[3.1.0]hexane- 3-carboxamide | * | 1.65 | 567 |
| 24 | 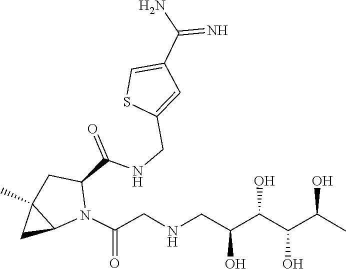 [SMILES: C[C@H](O)[C@H](O)[C@@H](O)[C@@H](O)CNCC(=O)N1[C@H]2C[C@@]2(C)C[C@H]1C(=O)NCc1cc(C(=N)N)cs1] | (1S,3S,5S)-N-((4- carbamimidoylthiophen- 2-yl)methyl)-5-methyl- 2-(((2S,3S,4S,5S)- 2,3,4,5- tetrahydroxyhexyl) glycyl)-2- azabicyclo[3.1.0]hexane- 3-carboxamide | * | 0.16 | 242 |
| 25 | 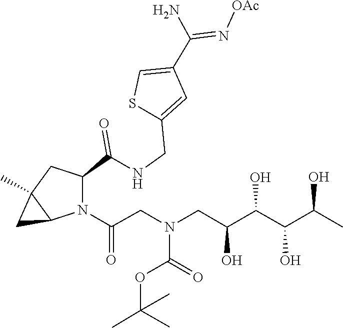 [SMILES: CC(=O)O/N=C(\N)c1csc(CNC(=O)[C@@H]2C[C@]3(C)C[C@@H]3N2C(=O)CN(C[C@H](O)[C@H](O)[C@@H](O)[C@H](C)O)C(=O)OC(C)(C)C)c1] | tert-butyl (2- ((1S,3S,5S)-3-(((4-((Z)- N′- acetoxycarbamimidoyl) thiophen-2- yl)methyl)carbamoyl)- 5-methyl-2- azabicyclo[3.1.0]hexan- 2-yl)-2- oxoethyl)((2S,3S,4S,5S)- 2,3,4,5- tetrahydroxyhexyl) carbamate | * | 1.20 | 642 |
| 26 | 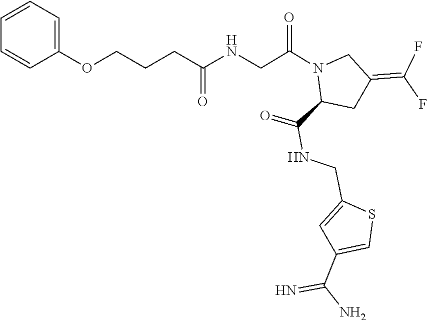 [SMILES: N=C(N)c1csc(CNC(=O)[C@@H]2CC(=C(F)F)CN2C(=O)CNC(=O)CCCOc2ccccc2)c1] | (S)-N-((4- carbamimidoylthiophen- 2-yl)methyl)-4- (difluoromethylene)-1- ((4- phenoxybutanoyl)glycyl) pyrrolidine-2- carboxamide | *** | 0.96 | 520 |
| 27 | 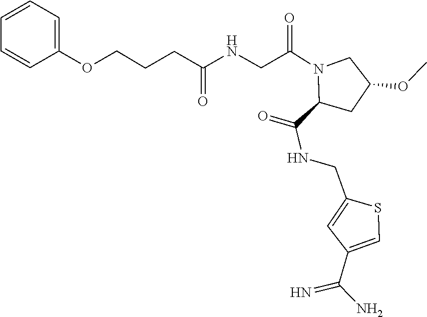 [SMILES: CO[C@@H]1C[C@@H](C(=O)NCc2cc(C(=N)N)cs2)N(C(=O)CNC(=O)CCCOc2ccccc2)C1] | (2S,4R)-N-((4- carbamimidoylthiophen- 2-yl)methyl)-4- methoxy-1-((4- phenoxybutanoyl)glycyl) pyrrolidine-2- carboxamide | *** | 0.71 | 502 |
| 28 | 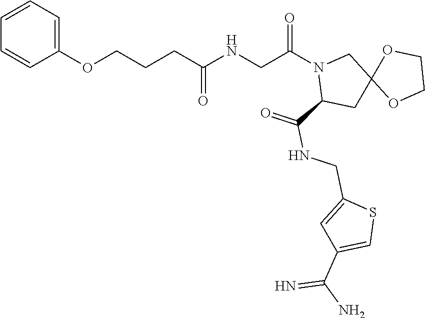 [SMILES: N=C(N)c1csc(CNC(=O)[C@@H]2CC3(CN2C(=O)CNC(=O)CCCOc2ccccc2)OCCO3)c1] | (S)-N-((4- carbamimidoylthiophen- 2-yl)methyl)-7-((4- phenoxybutanoyl)glycyl)- 1,4-dioxa-7- azaspiro[4.4]nonane-8- carboxamide | *** | 0.75 | 530 |
| 29 | 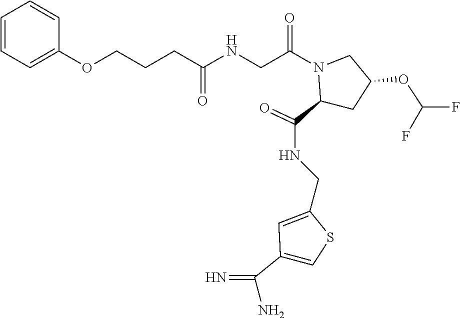 [SMILES: N=C(N)c1csc(CNC(=O)[C@@H]2C[C@@H](OC(F)F)CN2C(=O)CNC(=O)CCCOc2ccccc2)c1] | (2S,4R)-N-((4- carbamimidoylthiophen- 2-yl)methyl)-4- (difluoromethoxy)-1- ((4- phenoxybutanoyl)glycyl) pyrrolidine-2- carboxamide | *** | 0.93 | 538 |
| 30 | 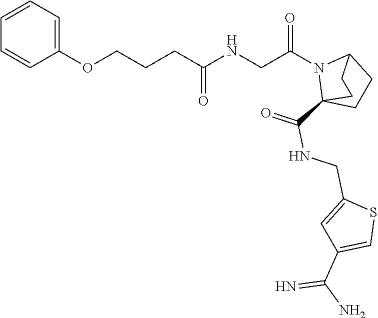 | N-((4- carbamimidoylthiophen- 2-yl)methyl)-7-((4- phenoxybutanoyl) glycyl)-7- azabicyclo[2.2.1] heptane-1-carboxamide | *** | 0.85 | 498 |
| 31 | 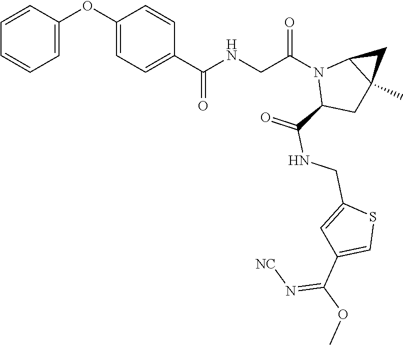 [SMILES: CO/C(=N/C#N)c1csc(CNC(=O)[C@@H]2C[C@]3(C)C[C@@H]3N2C(=O)CNC(=O)c2ccc(Oc3ccccc3)cc2)c1] | (E)-methyl N-cyano-5- (((1S,3S,5S)-5-methyl- 2-((4- phenoxybenzoyl)glycyl)- 2- azabicyclo[3.1.0]hexane-3- carboxamido)methyl) thiophene-3-carbimidate | * | 2.18 | 572 |
| 32 | 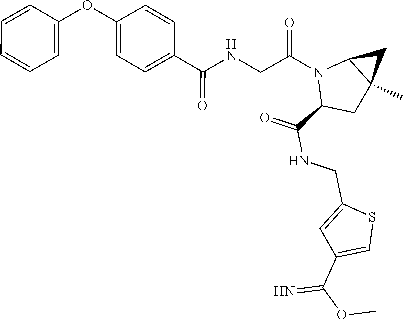 [SMILES: COC(=N)c1csc(CNC(=O)[C@@H]2C[C@]3(C)C[C@@H]3N2C(=O)CNC(=O)c2ccc(Oc3ccccc3)cc2)c1] | methyl 5-(((1S,3S,5S)- 5-methyl-2-((4- phenoxybenzoyl)glycyl)-2- azabicyclo[3.1.0]hexane-3- carboxamido)methyl) thiophene-3-carbimidate | ** | 1.33 | 547 |
| 33 | 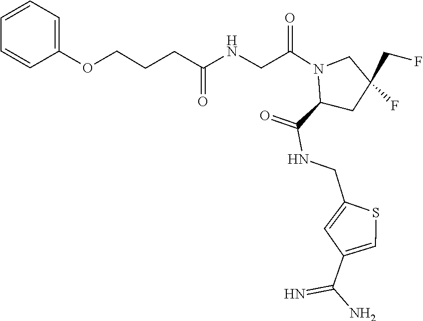 [SMILES: N=C(N)c1csc(CNC(=O)[C@@H]2C[C@](F)(CF)CN2C(=O)CNC(=O)CCCOc2ccccc2)c1] | (2S,4R)-N-((4- carbamimidoylthiophen-2- yl)methyl)-4-fluoro- 4-(fluoromethyl)-1-((4- phenoxybutanoyl)glycyl) pyrrolidine-2- carboxamide | *** | 0.86 | 522 |
| 34 | 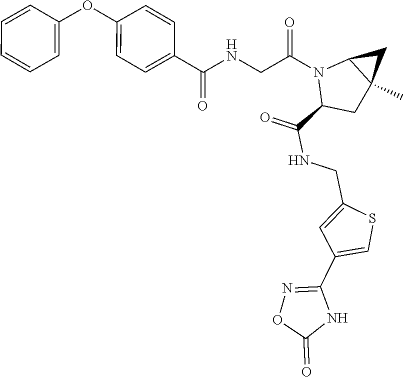 [SMILES: C[C@@]12C[C@@H]1N(C(=O)CNC(=O)c1ccc(Oc3ccccc3)cc1)[C@H](C(=O)NCc1cc(-c3noc(=O)[nH]3)cs1)C2] | (1S,3S,5S)-5-methyl-N- ((4-(5-oxo-4,5-dihydro- 1,2,4-oxadiazol-3- yl)thiophen-2- yl)methyl)-2-((4- phenoxybenzoyl)glycyl)-2- azabicyclo[3.1.0] hexane-3-carboxamide | * | 1.95 | 574 |
| 35 | 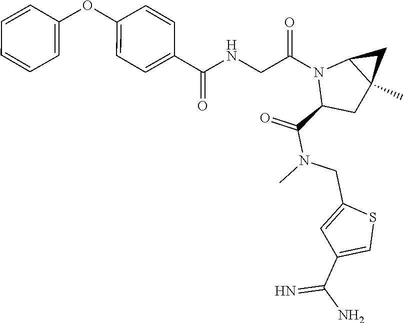 [SMILES: CN(Cc1cc(C(=N)N)cs1)C(=O)[C@@H]1C[C@]2(C)C[C@@H]2N1C(=O)CNC(=O)c1ccc(Oc2ccccc2)cc1] | (1S,3S,5S)-N-((4- carbamimidoylthiophen- 2-yl)methyl)-N,5- dimethyl-2-((4- phenoxybenzoyl)glycyl)- 2- azabicyclo[3.1.0]hexane- 3-carboxamide | | 1.29 | 546 |
| 36 | 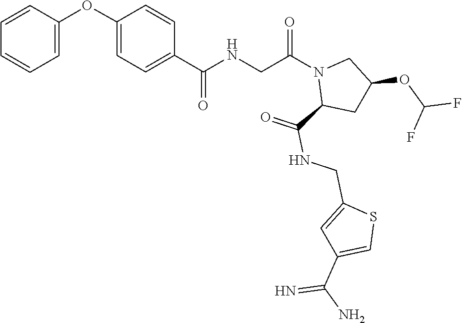 [SMILES: N=C(N)c1csc(CNC(=O)[C@@H]2C[C@H](OC(F)F)CN2C(=O)CNC(=O)c2ccc(Oc3ccccc3)cc2)c1] | (2S,4S)-N-((4- carbamimidoylthiophen- 2-yl)methyl)-4- (difluoromethoxy)-1- ((4- phenoxybenzoyl)glycyl) pyrrolidine-2- carboxamide | *** | 1.17 | 572 |
| 37 | 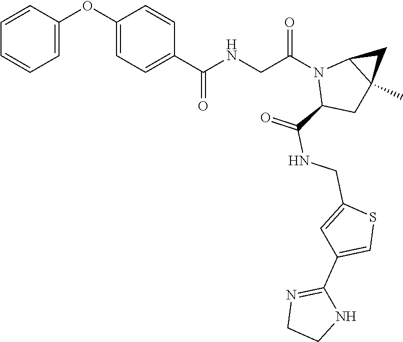 [SMILES: C[C@@]12C[C@@H]1N(C(=O)CNC(=O)c1ccc(Oc3ccccc3)cc1)[C@H](C(=O)NCc1cc(C3=NCCN3)cs1)C2] | (1S,3S,5S)-N-((4-(4,5- dihydro-1H-imidazol-2- yl)thiophen-2- yl)methyl)-5-methyl-2- ((4- phenoxybenzoyl)glycyl)- 2-azabicyclo[3.1.0] hexane-3-carboxamide | * | 1.12 | 558 |
| 38 | 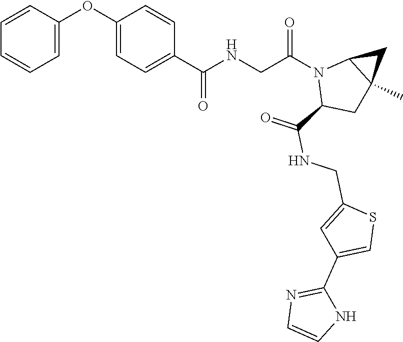 [SMILES: C[C@@]12C[C@@H]1N(C(=O)CNC(=O)c1ccc(Oc3ccccc3)cc1)[C@H](C(=O)NCc1cc(-c3ncc[nH]3)cs1)C2] | (1S,3S,5S)-N-((4-(1H- imidazol-2-yl)thiophen- 2-yl)methyl)-5-methyl- 2-((4- phenoxybenzoyl)glycyl)-2- azabicyclo[3.1.0]hexane- 3-carboxamide | *** | 1.30 | 556 |
| 39 | 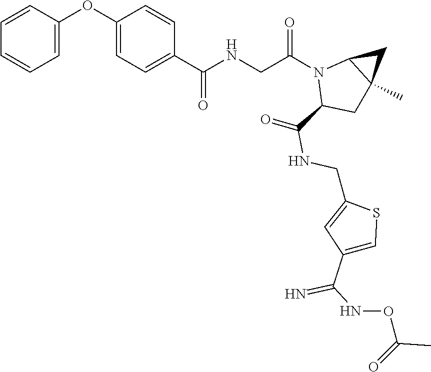 [SMILES: CC(=O)ONC(=N)c1csc(CNC(=O)[C@@H]2C[C@]3(C)C[C@@H]3N2C(=O)CNC(=O)c2ccc(Oc3ccccc3)cc2)c1] | (1S,3S,5S)-N-((4-(N- acetoxycarbamimidoyl) thiophen-2-yl)methyl)- 5-methyl-2-((4- phenoxybenzoyl)glycyl)-2- azabicyclo[3.1.0]hexane- 3-carboxamide | ** | 1.84 | 590 |
| 40 | 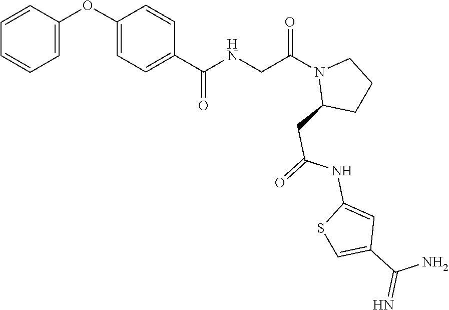 [SMILES: N=C(N)c1csc(NC(=O)C[C@@H]2CCCN2C(=O)CNC(=O)c2ccc(Oc3ccccc3)cc2)c1] | (S)-N-(2-(2-(2-((4- carbamimidoylthiophen- 2-yl)amino)-2- oxoethyl)pyrrolidin-1- yl)-2-oxoethyl)-4- phenoxybenzamide | * | 1.16 | 506 |
| 41 | 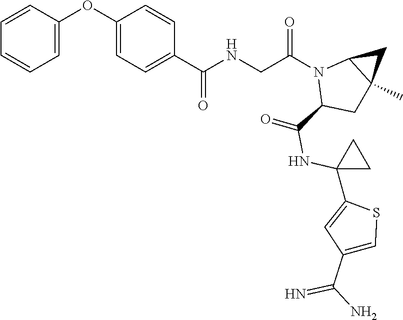 [SMILES: C[C@@]12C[C@@H]1N(C(=O)CNC(=O)c1ccc(Oc3ccccc3)cc1)[C@H](C(=O)NC1(c3cc(C(=N)N)cs3)CC1)C2] | (1S,3S,5S)-N-(1-(4- carbamimidoylthiophen- 2-yl)cyclopropyl)-5- methyl-2-((4- phenoxybenzoyl)glycyl)- 2- azabicyclo[3.1.0]hexane- 3-carboxamide | *** | 1.41 | 558 |
| 42 | 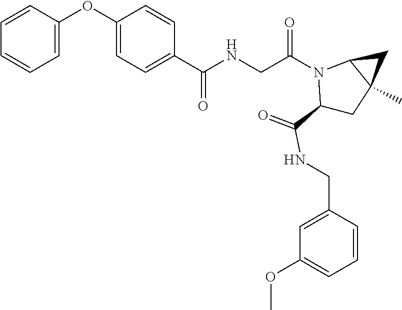 [SMILES: COc1cccc(CNC(=O)[C@@H]2C[C@]3(C)C[C@@H]3N2C(=O)CNC(=O)c2ccc(Oc3ccccc3)cc2)c1] | (1S,3S,5S)-N-(3- methoxybenzyl)-5- methyl-2-((4- phenoxybenzoyl)glycyl)-2- azabicyclo[3.1.0]hexane- 3-carboxamide | * | 2.49 | 514 |
| 43 | 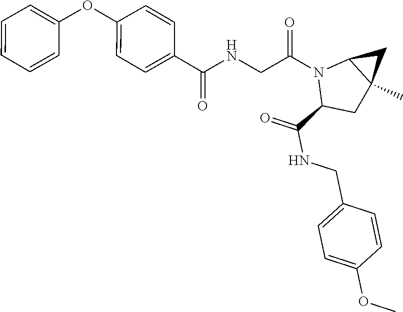 [SMILES: COc1ccc(CNC(=O)[C@@H]2C[C@]3(C)C[C@@H]3N2C(=O)CNC(=O)c2ccc(Oc3ccccc3)cc2)cc1] | (1S,3S,5S)-N-(4- methoxybenzyl)-5- methyl-2-((4- phenoxybenzoyl)glycyl)- 2- azabicyclo[3.1.0]hexane- 3-carboxamide | * | 2.46 | 514 |
| 44 | 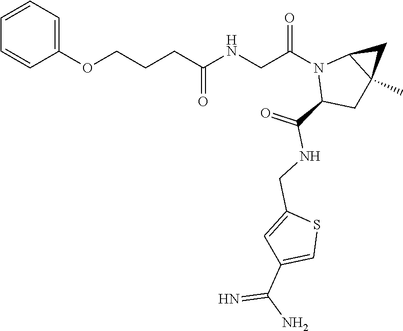 [SMILES: C[C@@]12C[C@@H]1N(C(=O)CNC(=O)CCCOc1ccccc1)[C@H](C(=O)NCc1cc(C(=N)N)cs1)C2] | (1S,3S,5S)-N-((4- carbamimidoylthiophen- 2-yl)methyl)-5-methyl- 2-((4- phenoxybutanoyl)glycyl)- 2- azabicyclo[3.1.0]hexane- 3-carboxamide | *** | 1.20 | 498 |
| 45 | 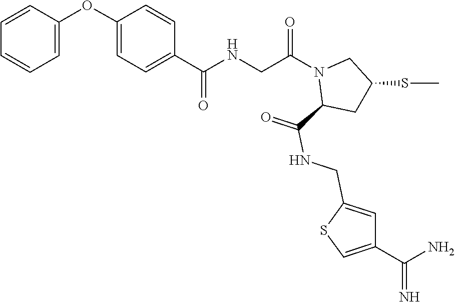 [SMILES: CS[C@@H]1C[C@@H](C(=O)NCc2cc(C(=N)N)cs2)N(C(=O)CNC(=O)c2ccc(Oc3ccccc3)cc2)C1] | (2S,4R)-N-((4- carbamimidoylthiophen- 2-yl)methyl)-4- (methylthio)-1-((4- phenoxybenzoyl)glycyl) pyrrolidine-2- carboxamide | *** | 1.47 | 552 |
| 46 | 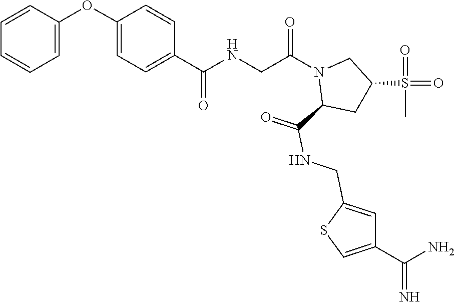 [SMILES: CS(=O)(=O)[C@@H]1C[C@@H](C(=O)NCc2cc(C(=N)N)cs2)N(C(=O)CNC(=O)c2ccc(Oc3ccccc3)cc2)C1] | (2S,4R)-N-((4- carbamimidoylthiophen- 2-yl)methyl)-4- (methylsulfonyl)-1-((4- phenoxybenzoyl)glycyl) pyrrolidine-2- carboxamide | *** | 1.28 | 584 |
| 47 | 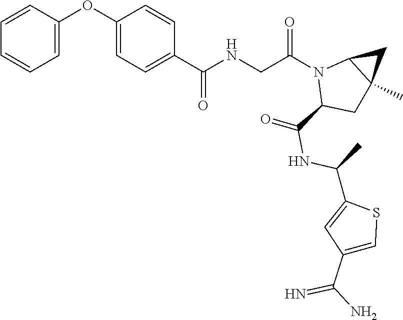 [SMILES: C[C@H](NC(=O)[C@@H]1C[C@]2(C)C[C@@H]2N1C(=O)CNC(=O)c1ccc(Oc2ccccc2)cc1)c1cc(C(=N)N)cs1] | (1S,3S,5S)-N-((S)-1-(4- carbamimidoylthiophen- 2-yl)ethyl)-5-methyl-2- ((4- phenoxybenzoyl)glycyl)- 2- azabicyclo[3.1.0]hexane- 3-carboxamide | *** | 1.64 | 546 |
| 48 | 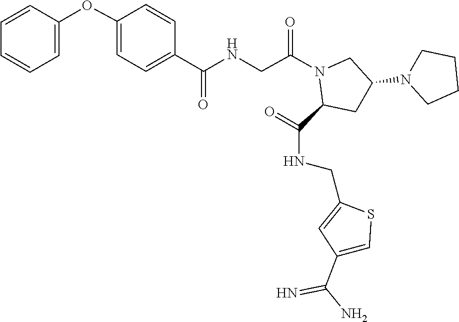 [SMILES: N=C(N)c1csc(CNC(=O)[C@@H]2C[C@@H](N3CCCC3)CN2C(=O)CNC(=O)c2ccc(Oc3ccccc3)cc2)c1] | (3′R,5′S)-N-((4- carbamimidoylthiophen- 2-yl)methyl)-1′-((4- phenoxybenzoyl)glycyl)- [1,3′-bipyrrolidine]-5′- carboxamide | *** | 1.03 | 575 |
| 49 | 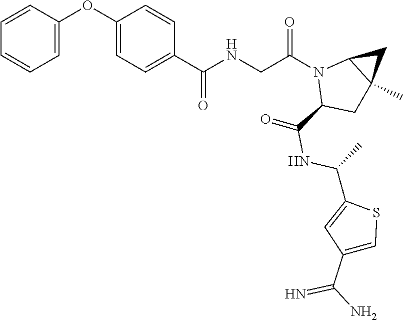 [SMILES: C[C@@H](NC(=O)[C@@H]1C[C@]2(C)C[C@@H]2N1C(=O)CNC(=O)c1ccc(Oc2ccccc2)cc1)c1cc(C(=N)N)cs1] | (1S,3S,5S)-N-((R)-1-(4- carbamimidoylthiophen- 2-yl)ethyl)-5-methyl-2- ((4- phenoxybenzoyl)glycyl)- 2- azabicyclo[3.1.0]hexane- 3-carboxamide | ** | 1.53 | 546 |
| 50 | 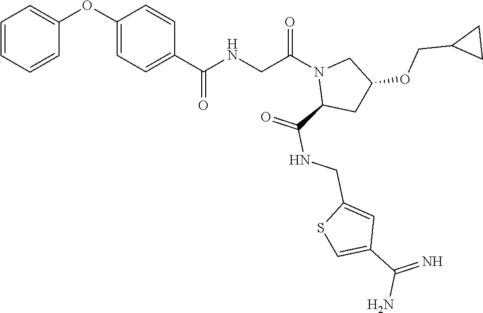 [SMILES: N=C(N)c1csc(CNC(=O)[C@@H]2C[C@@H](OCC3CC3)CN2C(=O)CNC(=O)c2ccc(Oc3ccccc3)cc2)c1] | (2S,4R)-N-((4- carbamimidoylthiophen- 2-yl)methyl)-4- (cyclopropylmethoxy)- 1-((4- phenoxybenzoyl)glycyl) pyrrolidine-2- carboxamide | *** | 1.48 | 576 |
| 51 | 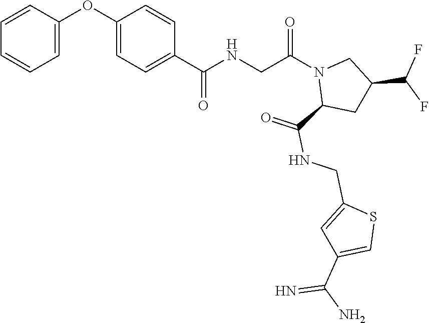 [SMILES: N=C(N)c1csc(CNC(=O)[C@@H]2C[C@H](C(F)F)CN2C(=O)CNC(=O)c2ccc(Oc3ccccc3)cc2)c1] | (2S,4S)-N-((4- carbamimidoylthiophen- 2-yl)methyl)-4- (difluoromethyl)-1-((4- phenoxybenzoyl)glycyl) pyrrolidine-2- carboxamide | *** | 1.38 | 556 |
| 52 | 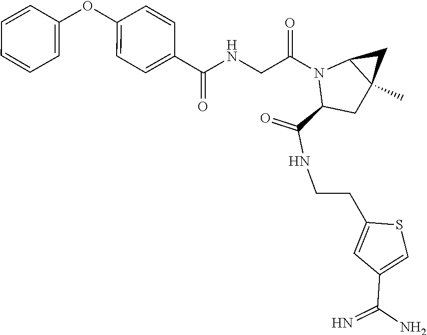 [SMILES: C[C@@]12C[C@@H]1N(C(=O)CNC(=O)c1ccc(Oc3ccccc3)cc1)[C@H](C(=O)NCCc1cc(C(=N)N)cs1)C2] | (1S,3S,5S)-N-(2-(4- carbamimidoylthiophen- 2-yl)ethyl)-5-methyl-2- ((4- phenoxybenzoyl)glycyl)- 2- azabicyclo[3.1.0]hexane- 3-carboxamide | *** | 1.50 | 546 |
| 53 | 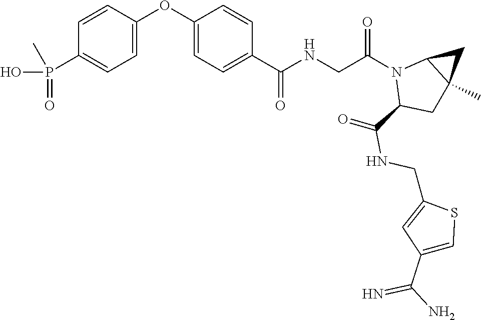 [SMILES: C[C@@]12C[C@@H]1N(C(=O)CNC(=O)c1ccc(Oc3ccc(P(C)(=O)O)cc3)cc1)[C@H](C(=O)NCc1cc(C(=N)N)cs1)C2] | (4-(4-((2-((1S,3S,5S)-3- (((4- carbamimidoylthiophen- 2- yl)methyl)carbamoyl)- 5-methyl-2- azabicyclo[3.1.0]hexan- 2-yl)-2- oxoethyl)carbamoyl) phenoxy)phenyl)(methyl) phosphinic acid | ** | 0.97 | 610 |
| 54 | 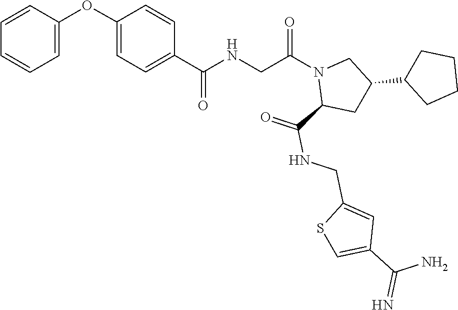 [SMILES: N=C(N)c1csc(CNC(=O)[C@@H]2C[C@@H](C3CCCC3)CN2C(=O)CNC(=O)c2ccc(Oc3ccccc3)cc2)c1] | (2S,4S)-N-((4- carbamimidoylthiophen- 2-yl)methyl)-4- cyclopentyl-1-((4- phenoxybenzoyl)glycyl) pyrrolidine-2- carboxamide | *** | 1.73 | 574 |
| 55 | 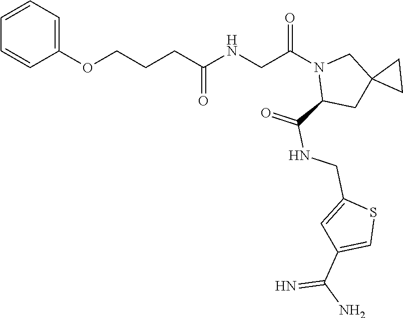 [SMILES: N=C(N)c1csc(CNC(=O)[C@@H]2CC3(CC3)CN2C(=O)CNC(=O)CCCOc2ccccc2)c1] | (S)-N-((4- carbamimidoylthiophen- 2-yl)methyl)-5-((4- phenoxybutanoyl)glycyl)- 5- azaspiro[2.4]heptane- 6-carboxamide | *** | 1.06 | 498 |
| 56 | 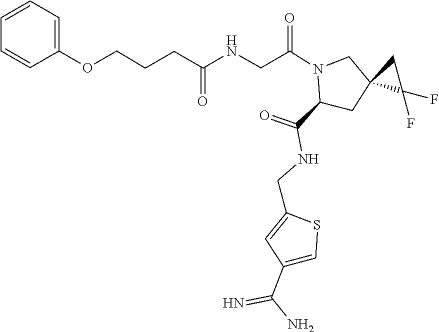 [SMILES: N=C(N)c1csc(CNC(=O)[C@@H]2C[C@@]3(CN2C(=O)CNC(=O)CCCOc2ccccc2)CC3(F)F)c1] | (3S,6S)-N-((4- carbamimidoylthiophen- 2-yl)methyl)-1,1- difluoro-5-((4- phenoxybutanoyl)glycyl)- 5- azaspiro[2.4]heptane- 6-carboxamide | *** | 1.18 | 534 |
| 57 | 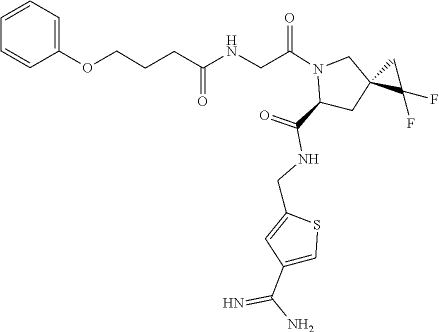 [SMILES: N=C(N)c1csc(CNC(=O)[C@@H]2C[C@]3(CN2C(=O)CNC(=O)CCCOc2ccccc2)CC3(F)F)c1] | (3R,6S)-N-((4- carbamimidoylthiophen- 2-yl)methyl)-1,1- difluoro-5-((4- phenoxybutanoyl) glycyl)-5- azaspiro[2.4]heptane- 6-carboxamide | *** | 1.11 | 534 |
| 58 | 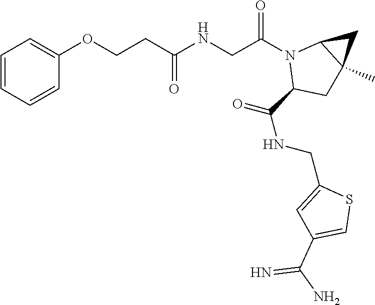 [SMILES: C[C@@]12C[C@@H]1N(C(=O)CNC(=O)CCOc1ccccc1)[C@H](C(=O)NCc1cc(C(=N)N)cs1)C2] | (1S,3S,5S)-N-((4- carbamimidoylthiophen- 2-yl)methyl)-5-methyl- 2-((3- phenoxypropanoyl)glycyl)- 2- azabicyclo[3.1.0]hexane- 3-carboxamide | *** | 1.02 | 484 |
| 59 | 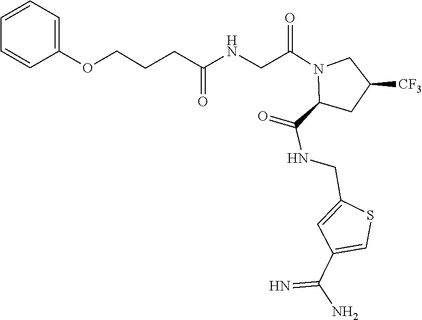 [SMILES: N=C(N)c1csc(CNC(=O)[C@@H]2C[C@H](C(F)(F)F)CN2C(=O)CNC(=O)CCCOc2ccccc2)c1] | (2S,4S)-N-((4- carbamimidoylthiophen- 2-yl)methyl)-1-((4- phenoxybutanoyl)glycyl)- 4- (trifluoromethyl) pyrrolidine- 2-carboxamide | *** | 1.17 | 540 |
| 60 | 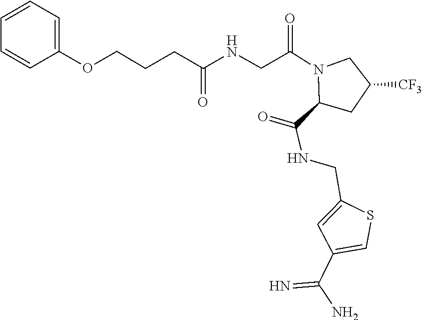 [SMILES: N=C(N)c1csc(CNC(=O)[C@@H]2C[C@@H](C(F)(F)F)CN2C(=O)CNC(=O)CCCOc2ccccc2)c1] | (2S,4R)-N-((4- carbamimidoylthiophen- 2-yl)methyl)-1-((4- phenoxybutanoyl)glycyl)-4- (trifluoromethyl) pyrrolidine- 2-carboxamide | *** | 1.19 | 540 |
| 61 | 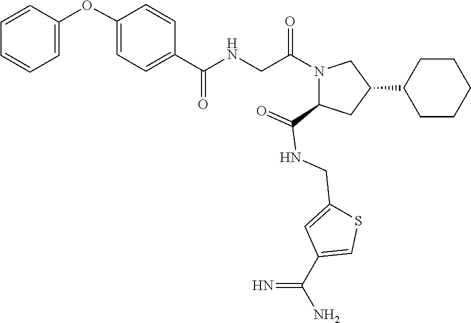 [SMILES: N=C(N)c1csc(CNC(=O)[C@@H]2C[C@@H](C3CCCCC3)CN2C(=O)CNC(=O)c2ccc(Oc3ccccc3)cc2)c1] | (2S,4S)-N-((4- carbamimidoylthiophen- 2-yl)methyl)-4- cyclohexyl-1-((4- phenoxybenzoyl)glycyl) pyrrolidine-2- carboxamide | ** | 1.84 | 588 |
| 62 | 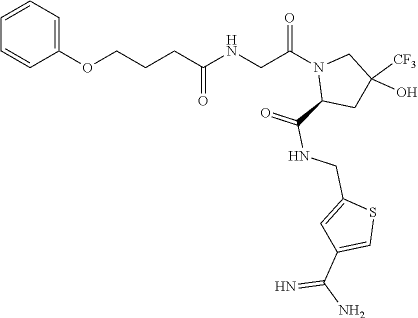 [SMILES: N=C(N)c1csc(CNC(=O)[C@@H]2CC(O)(C(F)(F)F)CN2C(=O)CNC(=O)CCCOc2ccccc2)c1] | (2S)-N-((4- carbamimidoylthiophen- 2-yl)methyl)-4- hydroxy-1-((4- phenoxybutanoyl)glycyl)-4- (trifluoromethyl) pyrrolidine- 2-carboxamide | *** | 1.07 | 556 |
| 63 | 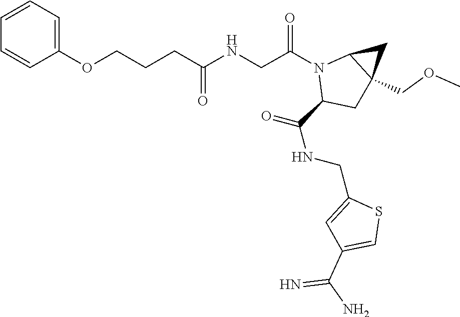 [SMILES: COC[C@@]12C[C@@H]1N(C(=O)CNC(=O)CCCOc1ccccc1)[C@H](C(=O)NCc1cc(C(=N)N)cs1)C2] | (1S,3S,5R)-N-((4- carbamimidoylthiophen- 2-yl)methyl)-5- (methoxymethyl)-2-((4- phenoxybutanoyl)glycyl)-2- azabicyclo[3.1.0]hexane- 3-carboxamide | *** | 1.03 | 528 |
| 64 | 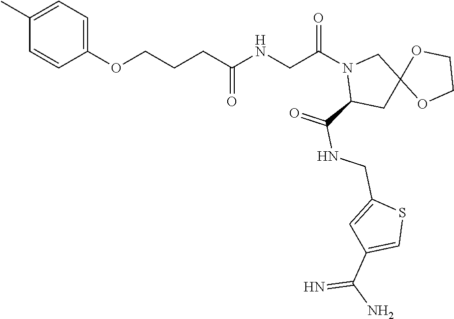 [SMILES: Cc1ccc(OCCCC(=O)NCC(=O)N2CC3(C[C@H]2C(=O)NCc2cc(C(=N)N)cs2)OCCO3)cc1] | (S)-N-((4- carbamimidoylthiophen- 2-yl)methyl)-7-((4-(p- tolyloxy)butanoyl)glycyl)- 1,4-dioxa-7- azaspiro[4.4]nonane-8- carboxamide | *** | 1.10 | 544 |
| 65 | 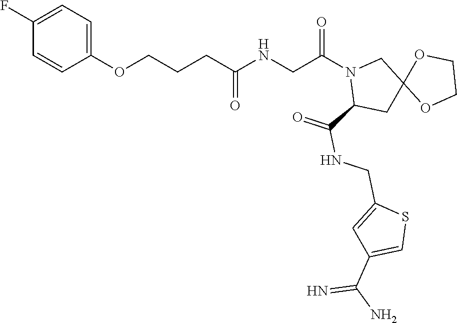 [SMILES: N=C(N)c1csc(CNC(=O)[C@@H]2CC3(CN2C(=O)CNC(=O)CCCOc2ccc(F)cc2)OCCO3)c1] | (S)-N-((4- carbamimidoylthiophen- 2-yl)methyl)-7-((4-(4- fluorophenoxy)butanoyl) glycyl)-1,4-dioxa-7- azaspiro[4.4]nonane-8- carboxamide | *** | 1.00 | 548 |
| 66 | 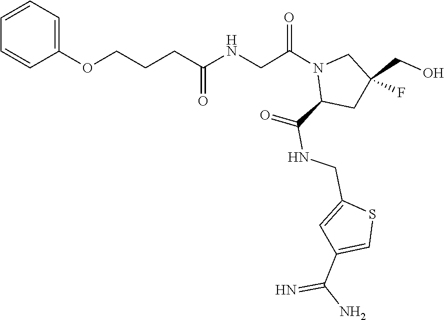 [SMILES: N=C(N)c1csc(CNC(=O)[C@@H]2C[C@](F)(CO)CN2C(=O)CNC(=O)CCCOc2ccccc2)c1] | (2S,4R)-N-((4- carbamimidoylthiophen- 2-yl)methyl)-4-fluoro- 4-(hydroxymethyl)-1- ((4- phenoxybutanoyl)glycyl) pyrrolidine-2- carboxamide | *** | 0.78 | 520 |
| 67 | 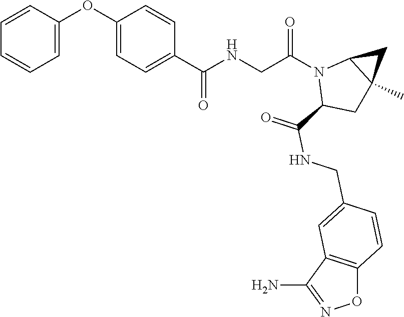 [SMILES: C[C@@]12C[C@@H]1N(C(=O)CNC(=O)c1ccc(Oc3ccccc3)cc1)[C@H](C(=O)NCc1ccc3onc(N)c3c1)C2] | (1S,3S,5S)-N-((3- aminobenzo[d]isoxazol- 5-yl)methyl)-5-methyl- 2-((4- phenoxybenzoyl)glycyl)-2- azabicyclo[3.1.0]hexane- 3-carboxamide | * | 2.10 | 540 |
| 68 | 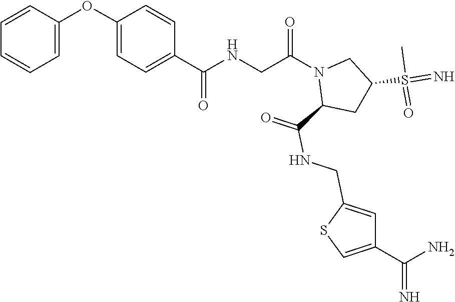 [SMILES: CS(=N)(=O)[C@@H]1C[C@@H](C(=O)NCc2cc(C(=N)N)cs2)N(C(=O)CNC(=O)c2ccc(Oc3ccccc3)cc2)C1] | (2S,4R)-N-((4- carbamimidoylthiophen- 2-yl)methyl)-4-((S)-S- methylsulfonimidoyl)-1- ((4- phenoxybenzoyl)glycyl) pyrrolidine-2- carboxamide | *** | 1.09 | 583 |
| 69 | 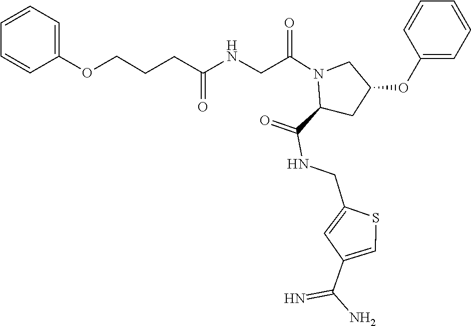 [SMILES: N=C(N)c1csc(CNC(=O)[C@@H]2C[C@@H](Oc3ccccc3)CN2C(=O)CNC(=O)CCCOc2ccccc2)c1] | (2S,4R)-N-((4- carbamimidoylthiophen- 2-yl)methyl)-4- phenoxy-1-((4- phenoxybutanoyl)glycyl) pyrrolidine-2- carboxamide | *** | 1.39 | 564 |
| 70 | 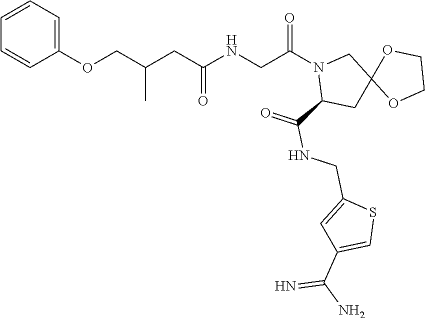 [SMILES: CC(COc1ccccc1)CC(=O)NCC(=O)N1CC2(C[C@H]1C(=O)NCc1cc(C(=N)N)cs1)OCCO2] | (8S)-N-((4- carbamimidoylthiophen- 2-yl)methyl)-7-((3- methyl-4- phenoxybutanoyl)glycyl)- 1,4-dioxa-7- azaspiro[4.4]nonane-8- carboxamide | *** | 1.07 | 544 |
| 71 | 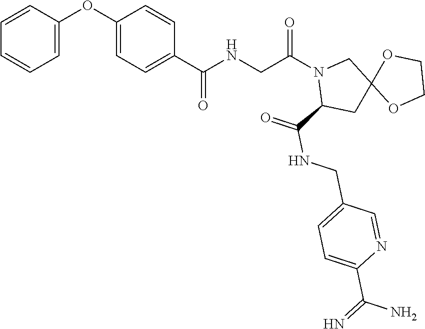 [SMILES: N=C(N)c1ccc(CNC(=O)[C@@H]2CC3(CN2C(=O)CNC(=O)c2ccc(Oc4ccccc4)cc2)OCCO3)cn1] | (S)-N-((6- carbamimidoylpyridin- 3-yl) methyl)-7-((4- phenoxybenzoyl)glycyl)- 1,4-dioxa-7- azaspiro[4.4]nonane-8- carboxamide | *** | 1.20 | 559 |
| 72 | 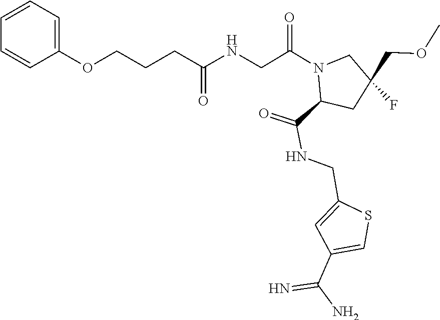 [SMILES: COC[C@@]1(F)C[C@@H](C(=O)NCc2cc(C(=N)N)cs2)N(C(=O)CNC(=O)CCCOc2ccccc2)C1] | (2S,4R)-N-((4- carbamimidoylthiophen- 2-yl)methyl)-4-fluoro- 4-(methoxymethyl)-1- ((4- phenoxybutanoyl)glycyl) pyrrolidine-2- carboxamide | *** | 1.00 | 534 |
| 73 | 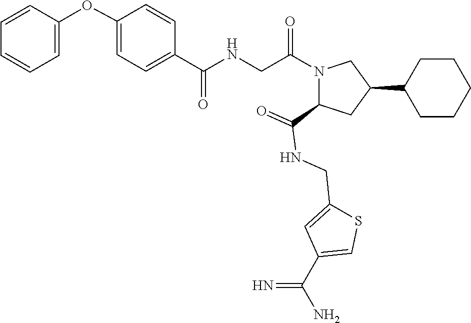 [SMILES: N=C(N)c1csc(CNC(=O)[C@@H]2C[C@H](C3CCCCC3)CN2C(=O)CNC(=O)c2ccc(Oc3ccccc3)cc2)c1] | (2S,4R)-N-((4- carbamimidoylthiophen- 2-yl)methyl)-4- cyclohexyl-1-((4- phenoxybenzoyl)glycyl) pyrrolidine-2- carboxamide | *** | 1.87 | 588 |
| 74 | 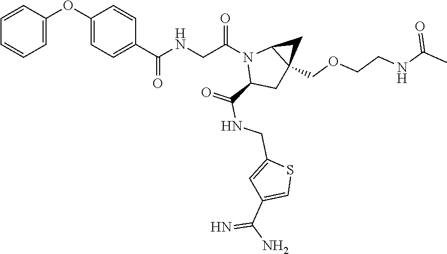 [SMILES: CC(=O)NCCOC[C@@]12C[C@@H]1N(C(=O)CNC(=O)c1ccc(Oc3ccccc3)cc1)[C@H](C(=O)NCc1cc(C(=N)N)cs1)C2] | (1S,3S,5R)-5-((2- acetamidoethoxy)meth yl)-N-((4- carbamimidoylthiophen- 2-yl)methyl)-2-((4- phenoxybenzoyl)glycyl)-2- azabicyclo[3.1.0]hexane- 3-carboxamide | *** | 1.24 | 633 |
| 75 | 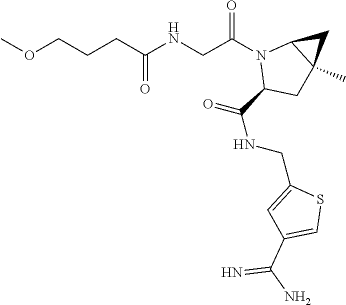 | (1S,3S,5S)-N-((4- carbamimidoylthiophen- 2-yl)methyl)-2-((4- methoxybutanoyl)glycyl)- 5-methyl-2- azabicyclo[3.1.0]hexane- 3-carboxamide | ** | 0.48 | 436 |
| 76 | 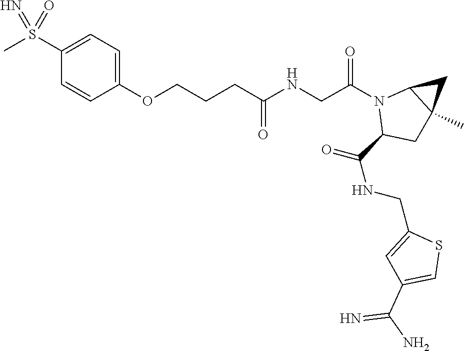 | (1S,3S,5S)-N-((4- carbamimidoylthiophen- 2-yl)methyl)-5-methyl- 2-((4-(4-(S- methylsulfonimidoyl) phenoxy)butanoyl)glycyl)- 2-azabicyclo[3.1.0]hexane- 3-carboxamide | ** | 0.56 | 575 |
| 77 | 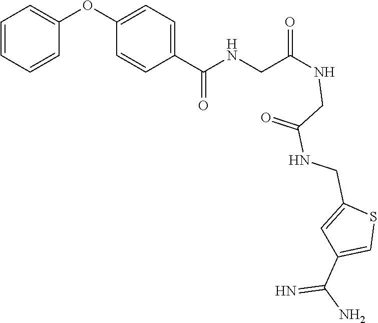 [SMILES: N=C(N)c1csc(CNC(=O)CNC(=O)CNC(=O)c2ccc(Oc3ccccc3)cc2)c1] | N-(2-((2-(((4- carbamimidoylthiophen- 2-yl)methyl)amino)-2- oxoethyl)amino)-2- oxoethyl)-4- phenoxybenzamide | ** | 1.18 | 466 |
| 78 | 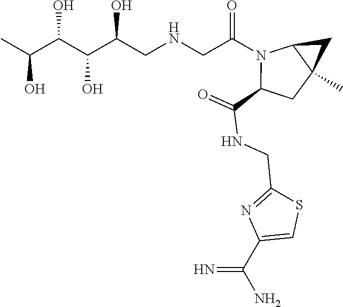 [SMILES: C[C@H](O)[C@H](O)[C@@H](O)[C@@H](O)CNCC(=O)N1[C@H]2C[C@@]2(C)C[C@H]1C(=O)NCc1nc(C(=N)N)cs1] | (1S,3S,5S)-N-((4- carbamimidoylthiazol- 2-yl)methyl)-5-methyl- 2-(((2S,3S,4S,5S)- 2,3,4,5- tetrahydroxyhexyl)glycyl)- 2-azabicyclo[3.1.0]hexane- 3-carboxamide | * | 0.23 | 485 |
| 79 | 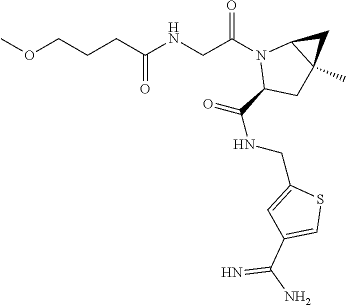 | ethyl 4-(4-((2- ((1S,3S,5S)-3-(((4- carbamimidoylthiophen-2- yl)methyl)carbamoyl)- 5-methyl-2- azabicyclo[3.1.0] hexan-2-yl)-2- oxoethyl)amino)-4- oxobutoxy)benzoate | *** | 1.35 | 570 |
| 80 | 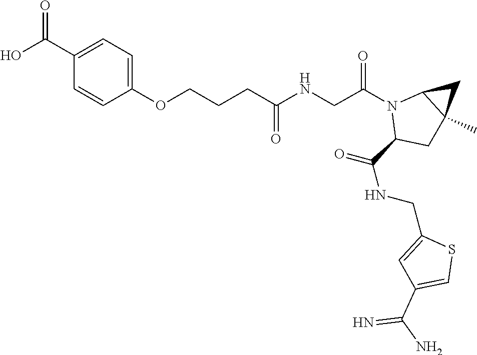 [SMILES: C[C@@]12C[C@@H]1N(C(=O)CNC(=O)CCCOc1ccc(C(=O)O)cc1)[C@H](C(=O)NCc1cc(C(=N)N)cs1)C2] | 4-(4-((2-((1S,3S,5S)-3- (((4- carbamimidoylthiophen-2- yl)methyl)carbamoyl)- 5-methyl-2- azabicyclo[3.1.0]hexan- 2-yl)-2- oxoethyl)amino)-4- oxobutoxy)benzoic acid | ** | 0.90 | 542 |
| 81 | 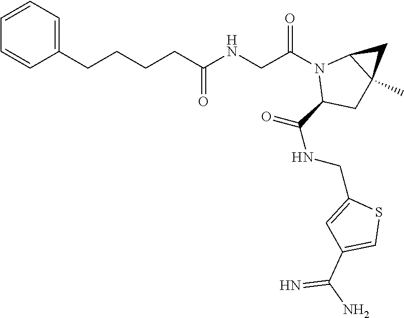 [SMILES: C[C@@]12C[C@@H]1N(C(=O)CNC(=O)CCCCc1ccccc1)[C@H](C(=O)NCc1cc(C(=N)N)cs1)C2] | (1S,3S,5S)-N-((4- carbamimidoylthiophen- 2-yl)methyl)-5-methyl- 2-((5- phenylpentanoyl)glycyl)-2- azabicyclo[3.1.0]hexane- 3-carboxamide | *** | 1.35 | 496 |
| 82 | 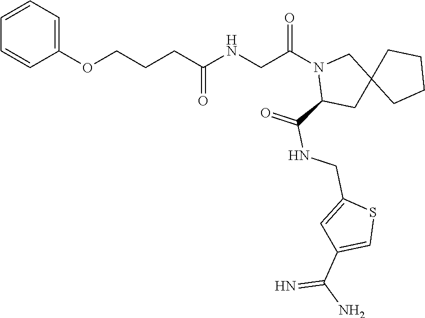 [SMILES: N=C(N)c1csc(CNC(=O)[C@@H]2CC3(CCCC3)CN2C(=O)CNC(=O)CCCOc2ccccc2)c1] | (S)-N-((4- carbamimidoylthiophen- 2-yl)methyl)-2-((4- phenoxybutanoyl)glycyl)-2- azaspiro[4.4]nonane-3- carboxamide | ** | 1.36 | 526 |
| 83 | 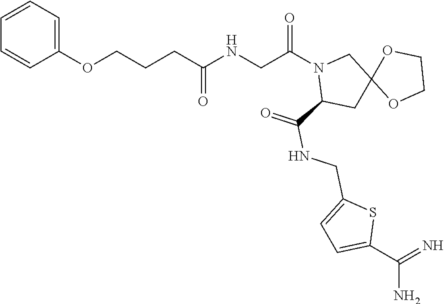 [SMILES: N=C(N)c1ccc(CNC(=O)[C@@H]2CC3(CN2C(=O)CNC(=O)CCCOc2ccccc2)OCCO3)s1] | (S)-N-((5- carbamimidoylthiophen- 2-yl)methyl)-7-((4- phenoxybutanoyl)glycyl)= 1,4-dioxa-7- azaspiro[4.4]nonane-8- carboxamide | *** | 0.96 | 530 |
| 84 | 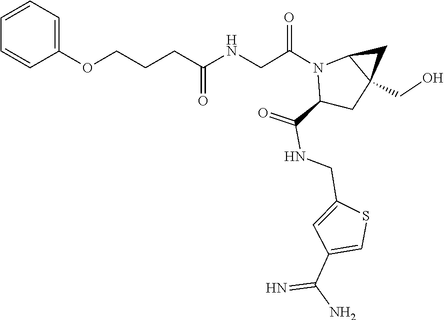 [SMILES: N=C(N)c1csc(CNC(=O)[C@@H]2C[C@]3(CO)C[C@@H]3N2C(=O)CNC(=O)CCCOc2ccccc2)c1] | (1S,3S,5R)-N-((4- carbamimidoylthiophen- 2-yl)methyl)-5- (hydroxymethyl)-2-((4- phenoxybutanoyl)glycyl)- 2-azabicyclo[3.1.0]hexane- 3-carboxamide | ** | 0.85 | 514 |
| 85 | 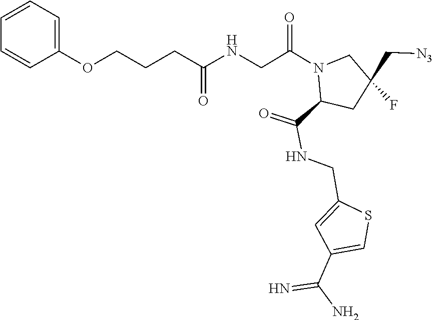 [SMILES: [N-]=[N+]=NC[C@@]1(F)C[C@@H](C(=O)NCc2cc(C(=N)N)cs2)N(C(=O)CNC(=O)CCCOc2ccccc2)C1] | (2S,4R)-4- (azidomethyl)-N-((4- carbamimidoylthiophen- 2-yl)methyl)-4-fluoro- 1-((4- phenoxybutanoyl)glycyl) pyrrolidine-2- carboxamide | *** | 1.16 | 545 |
| 86 | 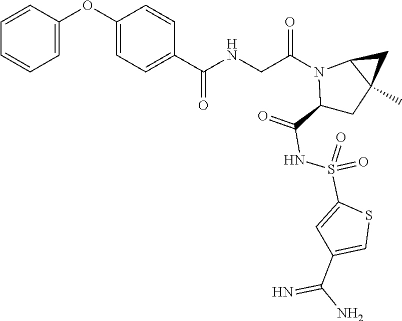 [SMILES: C[C@@]12C[C@@H]1N(C(=O)CNC(=O)c1ccc(Oc3ccccc3)cc1)[C@H](C(=O)NS(=O)(=O)c1cc(C(=N)N)cs1)C2] | (1S,3S,5S)-N-((4- carbamimidoylthiophen- 2-yl)sulfonyl)-5- methyl-2-((4- phenoxybenzoyl)glycyl)-2- azabicyclo[3.1.0]hexane- 3-carboxamide | * | 1.58 | 582 |
| 87 | 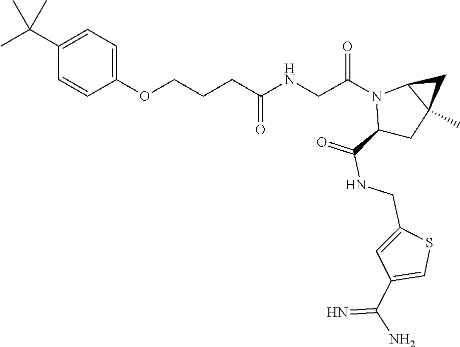 [SMILES: CC(C)(C)c1ccc(OCCCC(=O)NCC(=O)N2[C@H]3C[C@@]3(C)C[C@H]2C(=O)NCc2cc(C(=N)N)cs2)cc1] | (1S,3S,5S)-2-((4-(4- (tert- butyl)phenoxy)butanoyl) glycyl)-N-((4- carbamimidoylthiophen- 2-yl)methyl)-5-methyl- 2- azabicyclo[3.1.0]hexane- 3-carboxamide | *** | 1.71 | 554 |
| 88 | 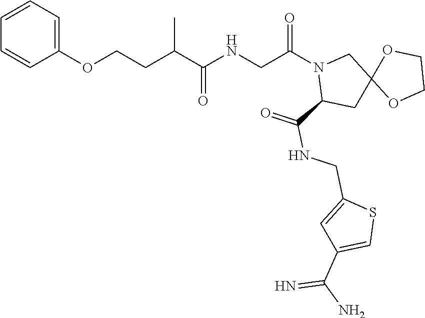 [SMILES: CC(CCOc1ccccc1)C(=O)NCC(=O)N1CC2(C[C@H]1C(=O)NCc1cc(C(=N)N)cs1)OCCO2] | (8S)-N-((4- carbamimidoylthiophen- 2-yl)methyl)-7-((2- methyl-4- phenoxybutanoyl)glycyl)- 1,4-dioxa-7- azaspiro[4.4]nonane-8- carboxamide | *** | 1.03 | 544 |
| 89 | 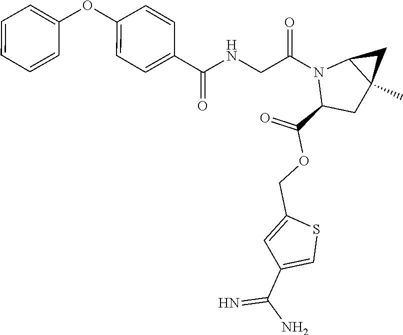 [SMILES: C[C@@]12C[C@@H]1N(C(=O)CNC(=O)c1ccc(Oc3ccccc3)cc1)[C@H](C(=O)OCc1cc(C(=N)N)cs1)C2] | (1S,3S,5S)-(4- carbamimidoylthiophen- 2-yl)methyl 5-methyl- 2-((4- phenoxybenzoyl)glycyl)- 2- azabicyclo[3.1.0]hexane- 3-carboxylate | *** | 1.57 | 533 |
| 90 | 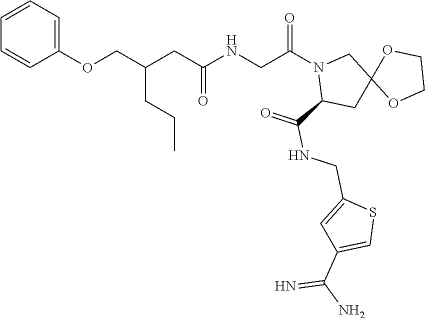 [SMILES: CCCC(COc1ccccc1)CC(=O)NCC(=O)N1CC2(C[C@H]1C(=O)NCc1cc(C(=N)N)cs1)OCCO2] | (8S)-N-((4- carbamimidoylthiophen- 2-yl)methyl)-7-((3- (phenoxymethyl)hexan oyl)glycyl)-1,4-dioxa-7- azaspiro[4.4]nonane-8- carboxamide | *** | 1.40 | 572 |
| 91 | 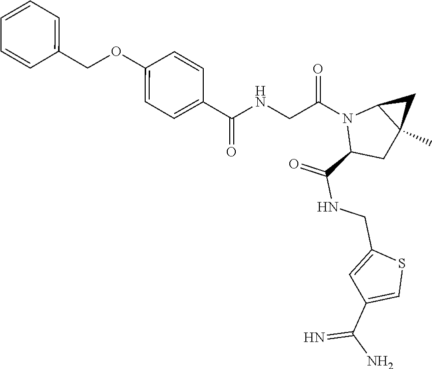 [SMILES: C[C@@]12C[C@@H]1N(C(=O)CNC(=O)c1ccc(OCc3ccccc3)cc1)[C@H](C(=O)NCc1cc(C(=N)N)cs1)C2] | (1S,3S,5S)-2-((4- (benzyloxy)benzoyl) glycyl)-N-((4- carbamimidoylthiophen- 2-yl)methyl)-5-methyl- 2- azabicyclo[3.1.0]hexane- 3-carboxamide | *** | 1.55 | 546 |
| 92 | 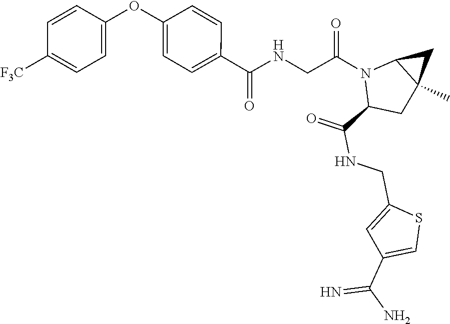 [SMILES: C[C@@]12C[C@@H]1N(C(=O)CNC(=O)c1ccc(Oc3ccc(C(F)(F)F)cc3)cc1)[C@H](C(=O)NCc1cc(C(=N)N)cs1)C2] | (1S,3S,5S)-N-((4- carbamimidoylthiophen- 2-yl)methyl)-5-methyl- 2-((4-(4- (trifluoromethyl)phenoxy) benzoyl)glycyl)-2- azabicyclo[3.1.0]hexane- 3-carboxamide | ** | 1.84 | 600 |
| 93 | 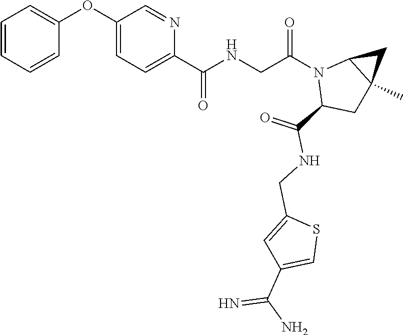 [SMILES: C[C@@]12C[C@@H]1N(C(=O)CNC(=O)c1ccc(Oc3ccccc3)cn1)[C@H](C(=O)NCc1cc(C(=N)N)cs1)C2] | (1S,3S,5S)-N-((4- carbamimidoylthiophen- 2-yl)methyl)-5-methyl- 2-((5- phenoxypicolinoyl)glyc yl)-2- azabicyclo[3.1.0]hexane- 3-carboxamide | *** | 1.47 | 533 |
| 94 | 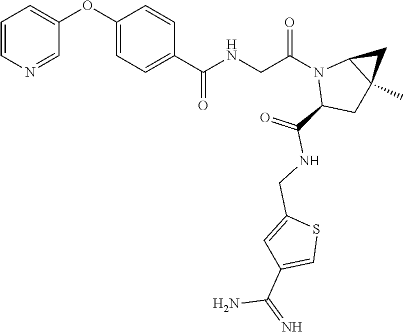 [SMILES: C[C@@]12C[C@@H]1N(C(=O)CNC(=O)c1ccc(Oc3cccnc3)cc1)[C@H](C(=O)NCc1cc(C(=N)N)cs1)C2] | (1S,3S,5S)-N-((4- carbamimidoylthiophen- 2-yl)methyl)-5-methyl- 2-((4-(pyridin-3- yloxy)benzoyl)glycyl)-2- azabicyclo[3.1.0]hexane- 3-carboxamide | *** | 0.95 | 533 |
| 95 | 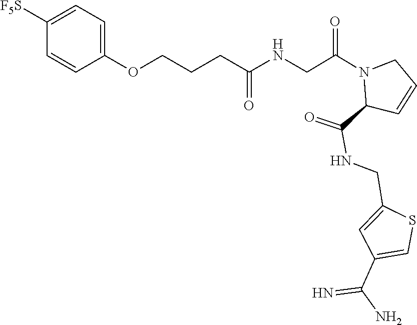 | (S)-N-((4- carbamimidoylthiophen- 2-yl)methyl)-1-((4-(4- (pentafluorolambda6- sulfanyl)phenoxy)butanoyl) glycyl)-2,5-dihydro- 1H-pyrrole-2- carboxamide | ** | 1.47 | 596 |
| 96 | 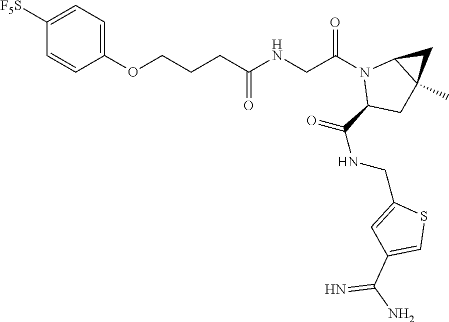 | (1S,3S,5S)-N-((4- carbamimidoylthiophen- 2-yl)methyl)-5-methyl- 2-((4-(4- (pentafluorolambda6- sulfanyl)phenoxy) butanoyl)glycyl)-2- azabicyclo[3.1.0]hexane- 3-carboxamide | *** | 1.61 | 624 |
| 97 | 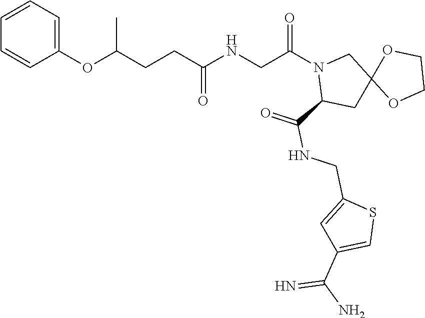 [SMILES: CC(CCC(=O)NCC(=O)N1CC2(C[C@H]1C(=O)NCc1cc(C(=N)N)cs1)OCCO2)Oc1ccccc1] | (8S)-N-((4- carbamimidoylthiophen- 2-yl)methyl)-7-((4- phenoxypentanoyl) glycyl)-1,4-dioxa-7- azaspiro[4.4]nonane-8- carboxamide | *** | 1.02 | 544 |
| 98 | 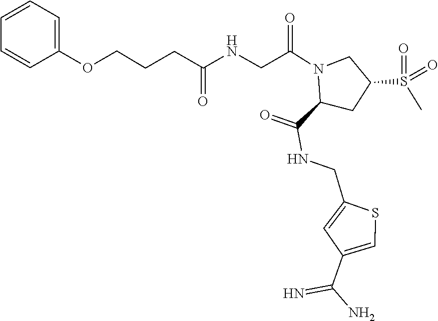 [SMILES: CS(=O)(=O)[C@@H]1C[C@@H](C(=O)NCc2cc(C(=N)N)cs2)N(C(=O)CNC(=O)CCCOc2ccccc2)C1] | (2S,4R)-N-((4- carbamimidoylthiophen- 2-yl)methyl)-4- (methylsulfonyl)-1-((4- phenoxybutanoyl)glycyl) pyrrolidine-2- carboxamide | *** | 0.84 | 550 |
| 99 | 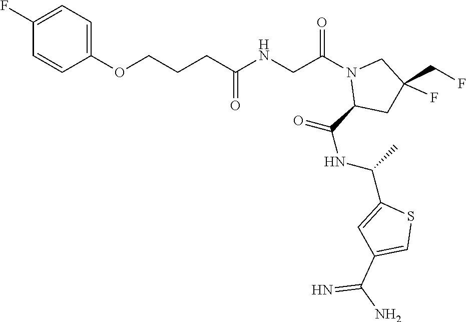 [SMILES: C[C@@H](NC(=O)[C@@H]1C[C@](F)(CF)CN1C(=O)CNC(=O)CCCOc1ccc(F)cc1)c1cc(C(=N)N)cs1] | (2S,4R)-N-((R)-1-(4- carbamimidoylthiophen- 2-yl)ethyl)-4-fluoro-4- (fluoromethyl)-1-((4-(4- fluorophenoxy)butanoyl) glycyl)pyrrolidine-2- carboxamide | *** | 1.15 | 554 |
| 100 | 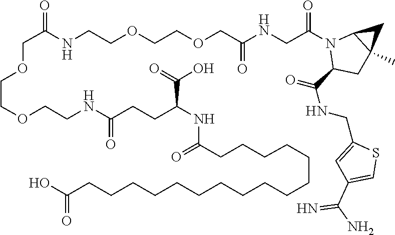 [SMILES: C[C@@]12C[C@@H]1N(C(=O)CNC(=O)COCCOCCNC(=O)COCCOCCNC(=O)CC[C@H](NC(=O)CCCCCCCCCCCCCCCCC(=O)O)C(=O)O)[C@H](C(=O)NCc1cc(C(=N)N)cs1)C2] | (S)-1-((1S,3S,5S)-3- (((4- carbamimidoylthiophen-2- yl)methyl)carbamoyl)- 5-methyl-2- azabicyclo[3.1.0]hexan -2-yl)-25-carboxy- 1,4,13,22,27-pentaoxo- 6,9,15,18-tetraoxa- 3,12,21,26- tetraazatetratetracontan- 44-oic acid | * | 1.93 | 1051 |
| 101 | 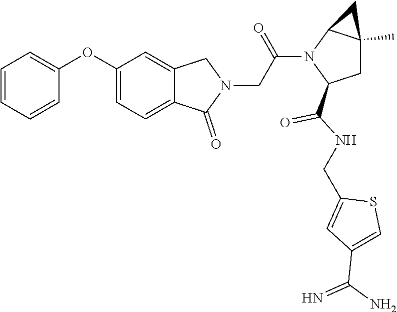 [SMILES: C[C@@]12C[C@@H]1N(C(=O)CN1Cc3cc(Oc4ccccc4)ccc3C1=O)[C@H](C(=O)NCc1cc(C(=N)N)cs1)C2] | (1S,3S,5S)-N-((4- carbamimidoylthiophen- 2-yl)methyl)-5-methyl- 2-(2-(1-oxo-5- phenoxyisoindolin-2- yl)acetyl)-2- azabicyclo[3.1.0]hexane- 3-carboxamide | *** | 1.51 | 544 |
| 102 | 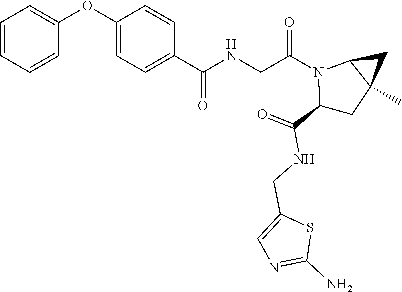 [SMILES: C[C@@]12C[C@@H]1N(C(=O)CNC(=O)c1ccc(Oc3ccccc3)cc1)[C@H](C(=O)NCc1cnc(N)s1)C2] | (1S,3S,5S)-N-((2- aminothiazol-5- yl)methyl)-5-methyl-2- ((4- phenoxybenzoyl)glycyl)-2- azabicyclo[3.1.0]hexane- 3-carboxamide | ** | 1.49 | 506 |
| 103 | 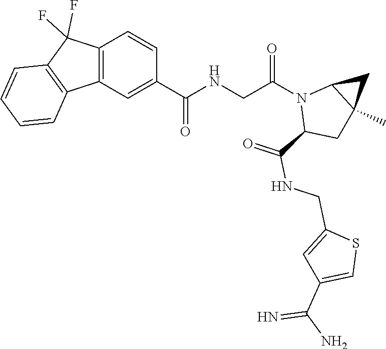 [SMILES: C[C@@]12C[C@@H]1N(C(=O)CNC(=O)c1ccc3c(c1)-c1ccccc1C3(F)F)[C@H](C(=O)NCc1cc(C(=N)N)cs1)C2] | (1S,3S,5S)-N-((4- carbamimidoylthiophen- 2-yl)methyl)-2-((9,9- difluoro-9H-fluorene-3- carbonyl) glycyl)-5- methyl-2- azabicyclo[3.1.0]hexane- 3-carboxamide | *** | 1.51 | 564 |
| 104 | 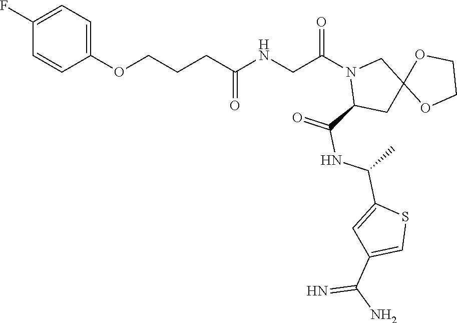 [SMILES: C[C@@H](NC(=O)[C@@H]1CC2(CN1C(=O)CNC(=O)CCCOc1ccc(F)cc1)OCCO2)c1cc(C(=N)N)cs1] | (S)-N-((R)-1-(4- carbamimidoylthiophen- 2-yl)ethyl)-7-((4-(4- fluorophenoxy)butanoyl) glycyl)-1,4-dioxa-7- azaspiro[4.4]nonane-8- carboxamide | *** | 1.06 | 562 |
| 105 | 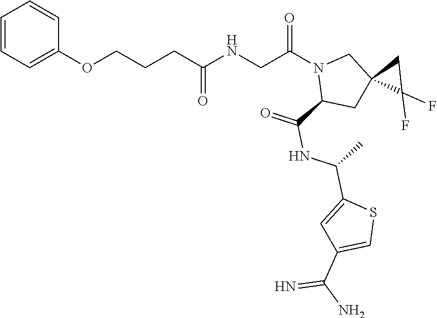 [SMILES: C[C@@H](NC(=O)[C@@H]1C[C@@]2(CN1C(=O)CNC(=O)CCCOc1ccccc1)CC2(F)F)c1cc(C(=N)N)cs1] | (3S,6S)-N-((R)-1-(4- carbamimidoylthiophen- 2-yl)ethyl)-1,1-difluoro- 5-((4- phenoxybutanoyl)glycyl)- 5-azaspiro[2.4]heptane- 6-carboxamide | ** | 1.22 | 548 |
| 106 | 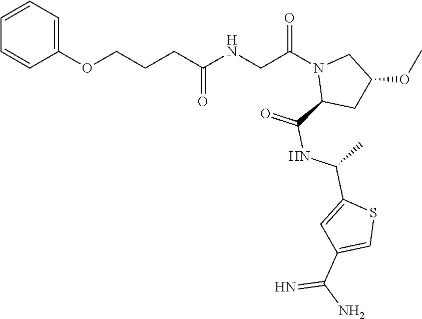 [SMILES: CO[C@@H]1C[C@@H](C(=O)N[C@H](C)c2cc(C(=N)N)cs2)N(C(=O)CNC(=O)CCCOc2ccccc2)C1] | (2S,4R)-N-((R)-1-(4- carbamimidoylthiophen- 2-yl)ethyl)-4-methoxy- 1-((4- phenoxybutanoyl)glycyl) pyrrolidine-2- carboxamide | *** | 0.95 | 516 |
| 107 | 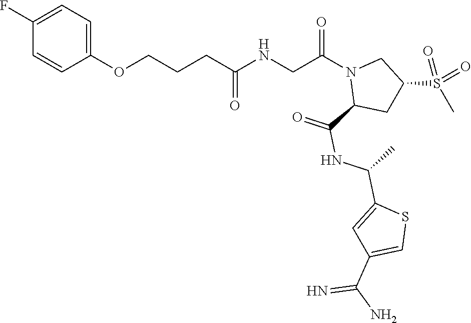 [SMILES: C[C@@H](NC(=O)[C@@H]1C[C@@H](S(C)(=O)=O)CN1C(=O)CNC(=O)CCCOc1ccc(F)cc1)c1cc(C(=N)N)cs1] | (2S,4R)-N-((R)-1-(4- carbamimidoylthiophen- 2-yl)ethyl)-1-((4-(4- fluorophenoxy)butanoyl) glycyl)-4- (methylsulfonyl) pyrrolidine- 2-carboxamide | *** | 0.98 | 582 |
| 108 | 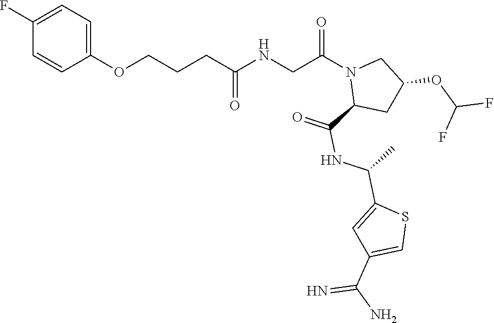 [SMILES: C[C@@H](NC(=O)[C@@H]1C[C@@H](OC(F)F)CN1C(=O)CNC(=O)CCCOc1ccc(F)cc1)c1cc(C(=N)N)cs1] | (2S,4R)-N-((R)-1-(4- carbamimidoylthiophen- 2-yl)ethyl)-4- (difluoromethoxy)-1- ((4-(4- fluorophenoxy)butanoyl) glycyl)pyrrolidine-2- carboxamide | *** | 1.22 | 570 |
| 109 | 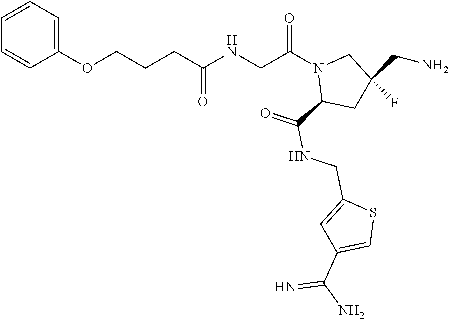 [SMILES: N=C(N)c1csc(CNC(=O)[C@@H]2C[C@](F)(CN)CN2C(=O)CNC(=O)CCCOc2ccccc2)c1] | (2S,4S)-4- (aminomethyl)-N-((4- carbamimidoylthiophen- 2-yl)methyl)-4-fluoro- 1-((4- phenoxybutanoyl)glycyl) pyrrolidine-2- carboxamide | *** | 0.37 | 519 |
| 110 | 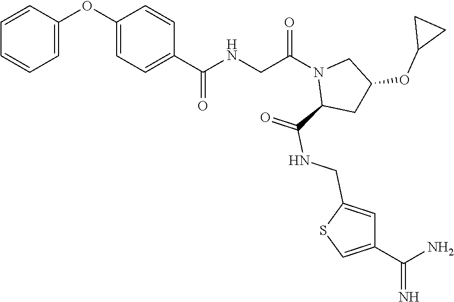 [SMILES: N=C(N)c1csc(CNC(=O)[C@@H]2C[C@@H](OC3CC3)CN2C(=O)CNC(=O)c2ccc(Oc3ccccc3)cc2)c1] | (2S,4R)-N-((4- carbamimidoylthiophen- 2-yl)methyl)-4- cyclopropoxy-1-((4- phenoxybenzoyl)glycyl) pyrrolidine-2- carboxamide | *** | 1.39 | 562 |
| 111 | 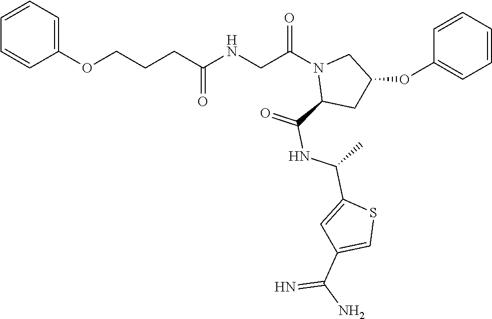 [SMILES: C[C@@H](NC(=O)[C@@H]1C[C@@H](Oc2ccccc2)CN1C(=O)CNC(=O)CCCOc1ccccc1)c1cc(C(=N)N)cs1] | (2S,4R)-N-((R)-1-(4- carbamimidoylthiophen- 2-yl)ethyl)-4-phenoxy- 1-((4- phenoxybutanoyl)glycyl) pyrrolidine-2- carboxamide | *** | 1.47 | 578 |
| 112 | 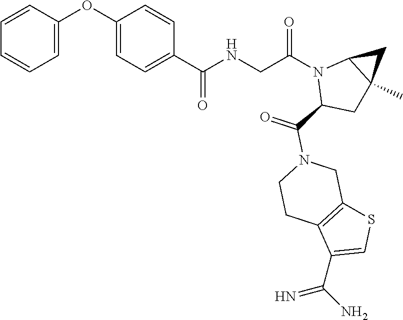 [SMILES: C[C@@]12C[C@@H]1N(C(=O)CNC(=O)c1ccc(Oc3ccccc3)cc1)[C@H](C(=O)N1CCc3c(C(=N)N)csc3C1)C2] | N-(2-((1S,3S,5S)-3-(3- carbamimidoyl-4,5,6,7- tetrahydrothieno[2,3- c]pyridine-6-carbonyl)- 5-methyl-2- azabicyclo[3.1.0]hexan- 2-yl)-2-oxoethyl)-4- phenoxybenzamide | ** | 1.47 | 558 |
| 113 | 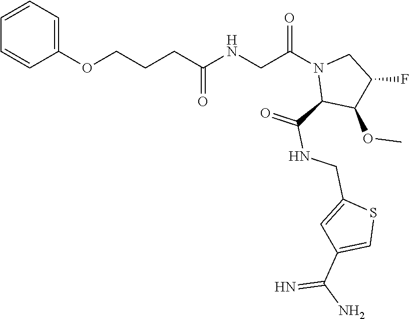 [SMILES: CO[C@H]1[C@@H](C(=O)NCc2cc(C(=N)N)cs2)N(C(=O)CNC(=O)CCCOc2ccccc2)C[C@@H]1F] | (2S,3S,4S)-N-((4- carbamimidoylthiophen- 2-yl)methyl)-4-fluoro- 3-methoxy-1-((4- phenoxybutanoyl)glycyl) pyrrolidine-2- carboxamide | ** | 0.94 | 520 |
| 114 | 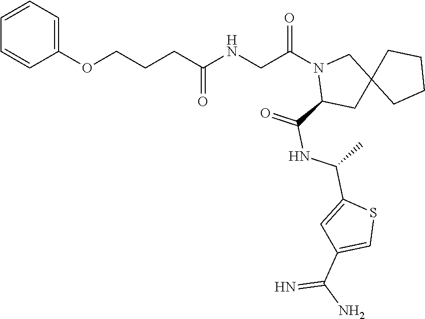 [SMILES: C[C@@H](NC(=O)[C@@H]1CC2(CCCC2)CN1C(=O)CNC(=O)CCCOc1ccccc1)c1cc(C(=N)N)cs1] | (S)-N-((R)-1-(4- carbamimidoylthiophen- 2-yl)ethyl)-2-((4- phenoxybutanoyl) glycyl)-2- azaspiro[4.4]nonane-3- carboxamide | *** | 1.38 | 540 |
| 115 | 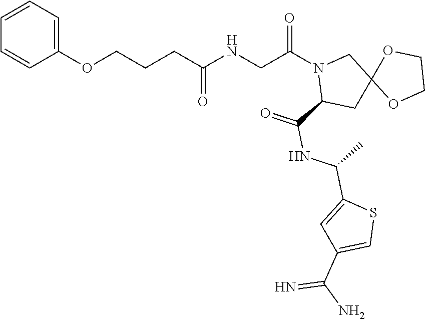 [SMILES: C[C@@H](NC(=O)[C@@H]1CC2(CN1C(=O)CNC(=O)CCCOc1ccccc1)OCCO2)c1cc(C(=N)N)cs1] | (S)-N-((R)-1-(4- carbamimidoylthiophen- 2-yl)ethyl)-7-((4- phenoxybutanoyl)glycyl)- 1,4-dioxa-7- azaspiro[4.4]nonane-8- carboxamide | *** | 0.99 | 544 |
| 116 | 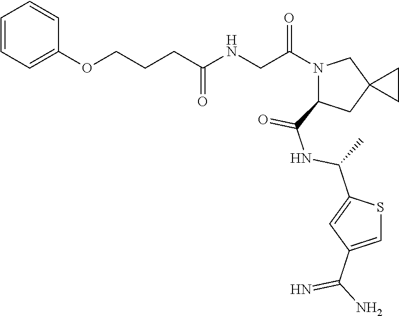 [SMILES: C[C@@H](NC(=O)[C@@H]1CC2(CC2)CN1C(=O)CNC(=O)CCCOc1ccccc1)c1cc(C(=N)N)cs1] | (S)-N-((R)-1-(4- carbamimidoylthiophen- 2-yl)ethyl)-5-((4- phenoxybutanoyl) glycyl)-5- azaspiro[2.4]heptane- 6-carboxamide | *** | 1.11 | 512 |
| 117 | 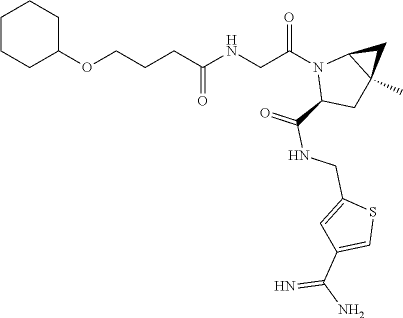 [SMILES: C[C@@]12C[C@@H]1N(C(=O)CNC(=O)CCCOC1CCCCC1)[C@H](C(=O)NCc1cc(C(=N)N)cs1)C2] | (1S,3S,5S)-N-((4- carbamimidoylthiophen- 2-yl)methyl)-2-((4- (cyclohexyloxy)butanoyl) glycyl)-5-methyl-2- azabicyclo[3.1.0]hexane- 3-carboxamide | *** | 1.18 | 504 |
| 118 | 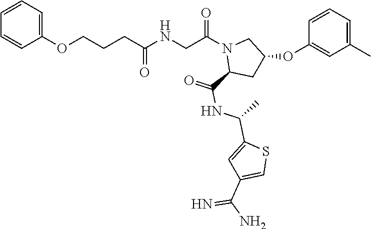 [SMILES: Cc1cccc(O[C@@H]2C[C@@H](C(=O)N[C@H](C)c3cc(C(=N)N)cs3)N(C(=O)CNC(=O)CCCOc3ccccc3)C2)c1] | (2S,4R)-N-((R)-1-(4- carbamimidoylthiophen- 2-yl)ethyl)-1-((4- phenoxybutanoyl)glycyl)- 4-(m- tolyloxy)pyrrolidine-2- carboxamide | *** | 1.56 | 592 |
| 119 | 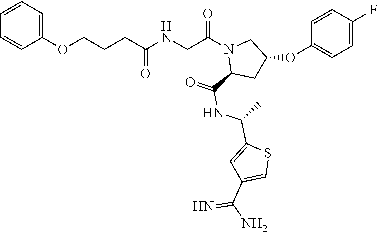 [SMILES: C[C@@H](NC(=O)[C@@H]1C[C@@H](Oc2ccc(F)cc2)CN1C(=O)CNC(=O)CCCOc1ccccc1)c1cc(C(=N)N)cs1] | (2S,4R)-N-((R)-1-(4- carbamimidoylthiophen- 2-yl)ethyl)-4-(4- fluorophenoxy)-1-((4- phenoxybutanoyl)glycyl) pyrrolidine-2- carboxamide | *** | 1.48 | 596 |
| 120 | 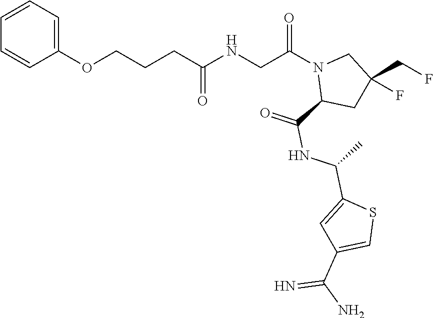 [SMILES: C[C@@H](NC(=O)[C@@H]1C[C@](F)(CF)CN1C(=O)CNC(=O)CCCOc1ccccc1)c1cc(C(=N)N)cs1] | (2S,4R)-N-((R)-1-(4- carbamimidoylthiophen- 2-yl)ethyl)-4-fluoro-4- (fluoromethyl)- 1-((4- phenoxybutanoyl)glycyl) pyrrolidine-2- carboxamide | | 1.06 | 536 |
| 121 | 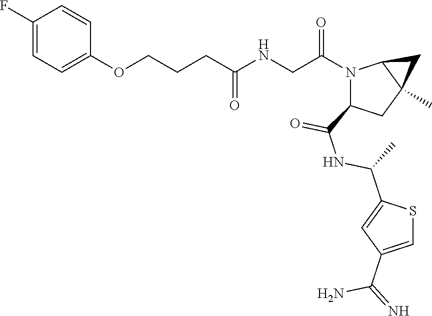 [SMILES: C[C@@H](NC(=O)[C@@H]1C[C@]2(C)C[C@@H]2N1C(=O)CNC(=O)CCCOc1ccc(F)cc1)c1cc(C(=N)N)cs1] | (1S,3S,5S)-N-((R)-1-(4- carbamimidoylthiophen- 2-yl)ethyl)-2-((4-(4- fluorophenoxy)butanoyl) glycyl)-5-methyl-2- azabicyclo[3.1.0]hexane- 3-carboxamide | ** | 1.24 | 530 |
| 122 | 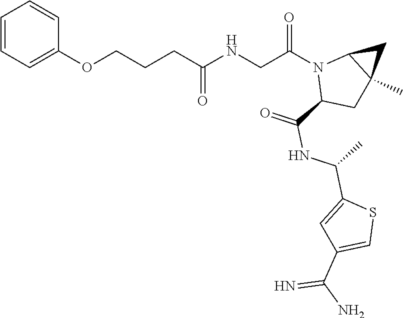 [SMILES: C[C@@H](NC(=O)[C@@H]1C[C@]2(C)C[C@@H]2N1C(=O)CNC(=O)CCCOc1ccccc1)c1cc(C(=N)N)cs1] | (1S,3S,5S)-N-((R)-1-(4- carbamimidoylthiophen- 2-yl)ethyl)-5-methyl-2- ((4- phenoxybutanoyl)glycyl)- 2- azabicyclo[3.1.0]hexane- 3-carboxamide | *** | 1.19 | 512 |
| 123 | 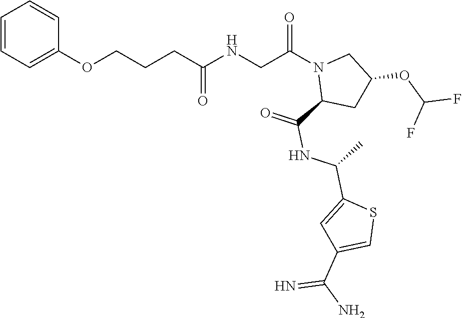 [SMILES: C[C@@H](NC(=O)[C@@H]1C[C@@H](OC(F)F)CN1C(=O)CNC(=O)CCCOc1ccccc1)c1cc(C(=N)N)cs1] | (2S,4R)-N-((R)-1-(4- carbamimidoylthiophen- 2-yl)ethyl)-4- (difluoromethoxy)-1-((4- phenoxybutanoyl)glycyl) pyrrolidine-2- carboxamide | *** | 1.15 | 552 |
| 124 | 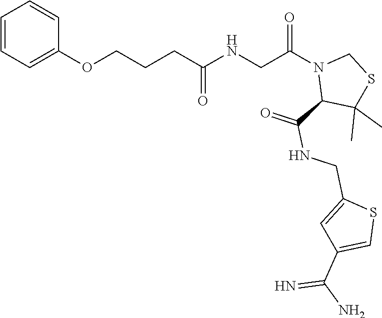 [SMILES: CC1(C)SCN(C(=O)CNC(=O)CCCOc2ccccc2)[C@@H]1C(=O)NCc1cc(C(=N)N)cs1] | (R)-N-((4- carbamimidoylthiophen- 2-yl)methyl)-5,5- dimethyl-3-((4- phenoxybutanoyl)glycyl) thiazolidine-4- carboxamide | ** | 1.03 | 518 |
| 125 | 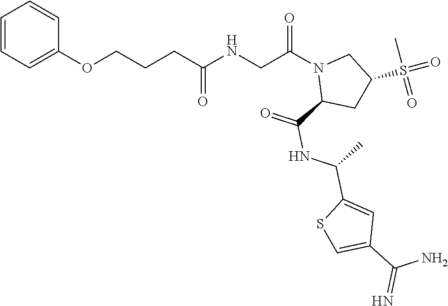 [SMILES: C[C@@H](NC(=O)[C@@H]1C[C@@H](S(C)(=O)=O)CN1C(=O)CNC(=O)CCCOc1ccccc1)c1cc(C(=N)N)cs1] | (2S,4R)-N-((R)-1-(4- carbamimidoylthiophen- 2-yl)ethyl)-4- (methylsulfonyl)-1-((4- phenoxybutanoyl)glycyl) pyrrolidine-2- carboxamide | *** | 0.89 | 564 |
| 126 | 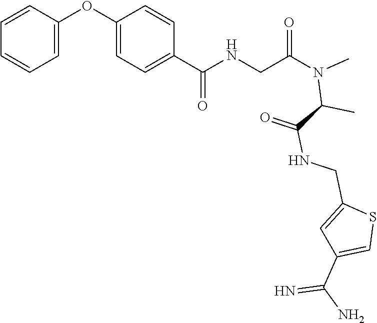 [SMILES: C[C@@H](C(=O)NCc1cc(C(=N)N)cs1)N(C)C(=O)CNC(=O)c1ccc(Oc2ccccc2)cc1] | (S)-N-(2-((1-(((4- carbamimidoylthiophen- 2-yl)methyl)amino)-1- oxopropan-2- yl)(methyl)amino)-2- oxoethyl)-4- phenoxybenzamide | *** | 1.32 | 494 |
| 127 | 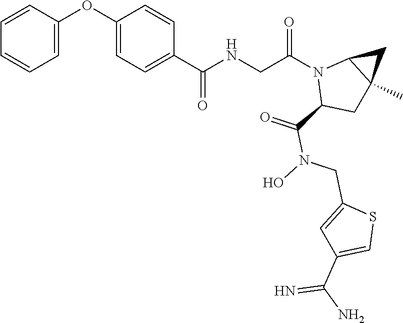 [SMILES: C[C@@]12C[C@@H]1N(C(=O)CNC(=O)c1ccc(Oc3ccccc3)cc1)[C@H](C(=O)N(O)Cc1cc(C(=N)N)cs1)C2] | (1S,3S,5S)-N-((4- carbamimidoylthiophen- 2-yl)methyl)-N- hydroxy-5-methyl-2-((4- phenoxybenzoyl)glycyl)- 2-azabicyclo[3.1.0]hexane- 3-carboxamide | * | 1.46 | 548 |
| 128 | 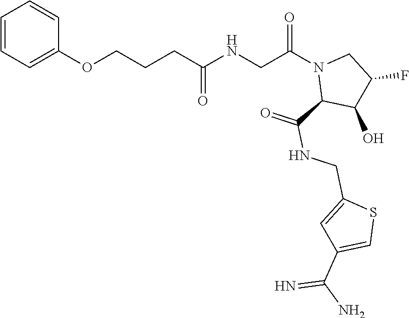 [SMILES: N=C(N)c1csc(CNC(=O)[C@@H]2[C@H](O)[C@@H](F)CN2C(=O)CNC(=O)CCCOc2ccccc2)c1] | (2S,3S,4S)-N-((4- carbamimidoylthiophen- 2-yl)methyl)-4-fluoro- 3-hydroxy-1-((4- phenoxybutanoyl)glycyl) pyrrolidine-2- carboxamide | *** | 0.86 | 506 |
| 129 | 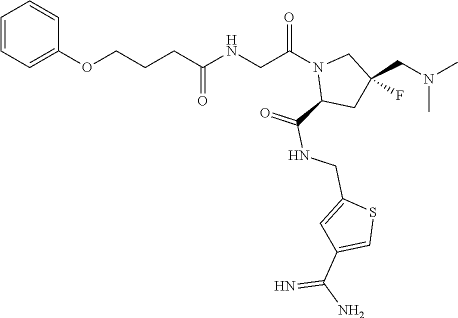 [SMILES: CN(C)C[C@@]1(F)C[C@@H](C(=O)NCc2cc(C(=N)N)cs2)N(C(=O)CNC(=O)CCCOc2ccccc2)C1] | (2S,4S)-N-((4- carbamimidoylthiophen- 2-yl)methyl)-4- ((dimethylamino)methyl)- 4-fluoro-1-((4- phenoxybutanoyl)glycyl) pyrrolidine-2- carboxamide | *** | 0.38 | 547 |
| 130 | 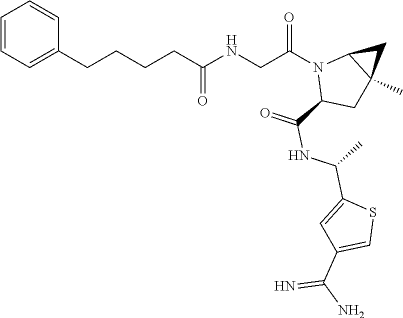 [SMILES: C[C@@H](NC(=O)[C@@H]1C[C@]2(C)C[C@@H]2N1C(=O)CNC(=O)CCCCc1ccccc1)c1cc(C(=N)N)cs1] | (1S,3S,5S)-N-((R)-1-(4- carbamimidoylthiophen- 2-yl)ethyl)-5- methyl-2-((5- phenylpentanoyl) glycyl)-2- azabicyclo[3.1.0]hexane- 3-carboxamide | *** | 1.40 | 510 |
| 131 | 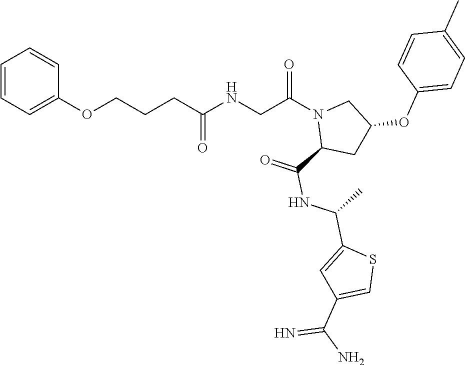 [SMILES: Cc1ccc(O[C@@H]2C[C@@H](C(=O)N[C@H](C)c3cc(C(=N)N)cs3)N(C(=O)CNC(=O)CCCOc3ccccc3)C2)cc1] | (2S,4R)-N-((R)-1-(4- carbamimidoylthiophen- 2-yl)ethyl)-1-((4- phenoxybutanoyl)glycyl)- 4-(p-tolyloxy) pyrrolidine-2- carboxamide | *** | 1.61 | 592 |
| 132 | 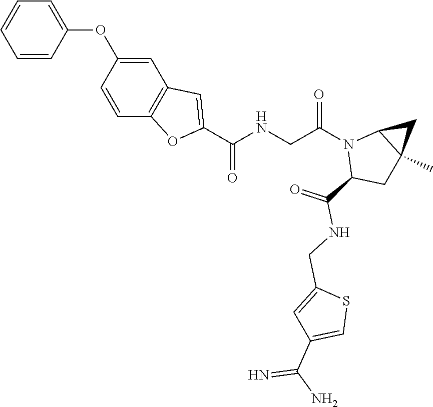 [SMILES: C[C@@]12C[C@@H]1N(C(=O)CNC(=O)c1cc3cc(Oc4ccccc4)ccc3o1)[C@H](C(=O)NCc1cc(C(=N)N)cs1)C2] | (1S,3S,5S)-N-((4- carbamimidoylthiophen- 2-yl)methyl)-5-methyl- 2-((5- phenoxybenzofuran-2- carbonyl)glycyl)-2- azabicyclo[3.1.0]hexane- 3-carboxamide | *** | 1.69 | 572 |
| 133 | 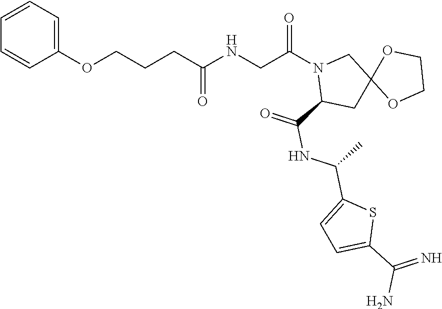 [SMILES: C[C@@H](NC(=O)[C@@H]1CC2(CN1C(=O)CNC(=O)CCCOc1ccccc1)OCCO2)c1ccc(C(=N)N)s1] | (S)-N-((R)-1-(5- carbamimidoylthiophen- 2-yl)ethyl)-7-((4- phenoxybutanoyl)glycyl)- 1,4-dioxa-7- azaspiro[4.4]nonane-8- carboxamide | *** | 0.99 | 544 |
| 134 | 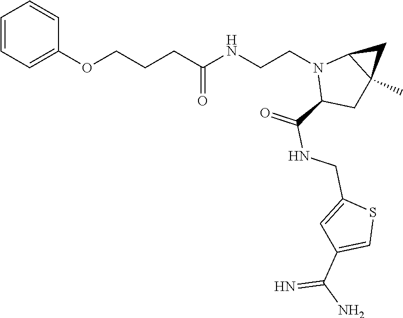 [SMILES: C[C@@]12C[C@@H]1N(CCNC(=O)CCCOc1ccccc1)[C@H](C(=O)NCc1cc(C(=N)N)cs1)C2] | (1S,3S,5S)-N-((4- carbamimidoylthiophen- 2-yl)methyl)-5-methyl- 2-(2-(4- phenoxybutanamido) ethyl)-2- azabicyclo[3.1.0]hexane- 3-carboxamide | * | 0.81 | 484 |
| 135 | 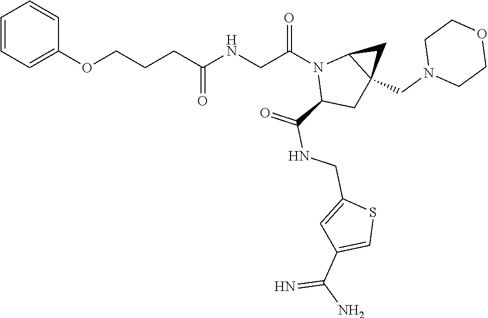 [SMILES: N=C(N)c1csc(CNC(=O)[C@@H]2C[C@]3(CN4CCOCC4)C[C@@H]3N2C(=O)CNC(=O)CCCOc2ccccc2)c1] | (1S,3S,5S)-N-((4- carbamimidoylthiophen- 2-yl)methyl)-5- (morpholinomethyl)- 2-((4- phenoxybutanoyl) glycyl)-2- azabicyclo[3.1.0]hexane- 3-carboxamide | *** | 0.81 | 583 |
| 136 | 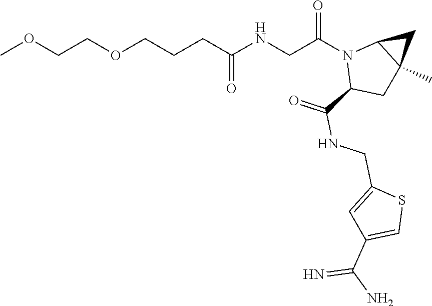 | (1S,3S,5S)-N-((4- carbamimidoylthiophen- 2-yl)methyl)-2-((4-(2- methoxyethoxy)butano yl) glycyl)-5-methyl-2- azabicyclo[3.1.0]hexane- 3-carboxamide | ** | 0.53 | 480 |
| 137 | 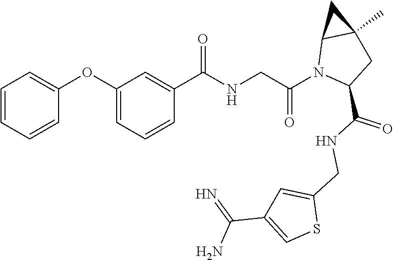 [SMILES: C[C@@]12C[C@@H]1N(C(=O)CNC(=O)c1cccc(Oc3ccccc3)c1)[C@H](C(=O)NCc1cc(C(=N)N)cs1)C2] | (1S,3S,5S)-N-((4- carbamimidoylthiophen- 2-yl)methyl)-5-methyl- 2-((3- phenoxybenzoyl)glycyl)-2- azabicyclo[3.1.0]hexane- 3-carboxamide | *** | 1.49 | 532 |
| 138 | 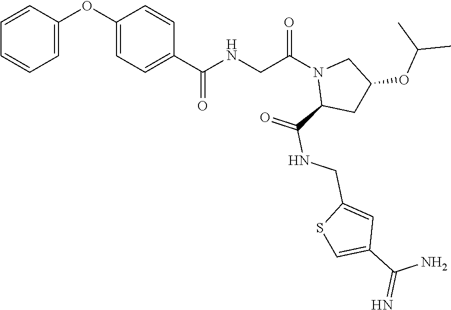 [SMILES: CC(C)O[C@@H]1C[C@@H](C(=O)NCc2cc(C(=N)N)cs2)N(C(=O)CNC(=O)c2ccc(Oc3ccccc3)cc2)C1] | (2S,4R)-N-((4- carbamimidoylthiophen- 2-yl)methyl)-4- isopropoxy-1-((4- phenoxybenzoyl)glycyl) pyrrolidine-2- carboxamide | *** | 1.43 | 564 |
| 139 | 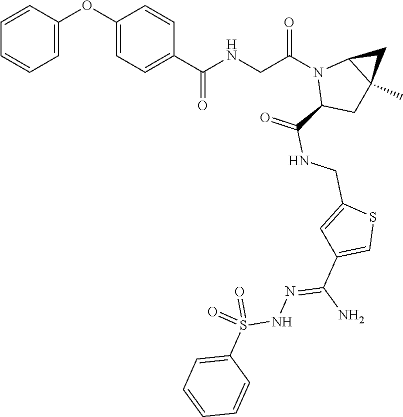 [SMILES: C[C@@]12C[C@@H]1N(C(=O)CNC(=O)c1ccc(Oc3ccccc3)cc1)[C@H](C(=O)NCc1cc(/C(N)=N/NS(=O)(=O)c3ccccc3)cs1)C2] | (1S,3S,5S)-5-methyl-2- ((4- phenoxybenzoyl)glycyl)- N-((4-((Z)-N′- (phenylsulfonyl)carbam ohydrazonoyl)thiophen- 2-yl)methyl)-2- azabicyclo[3.1.0]hexane- 3-carboxamide | * | 1.79 | 687 |
| 140 | 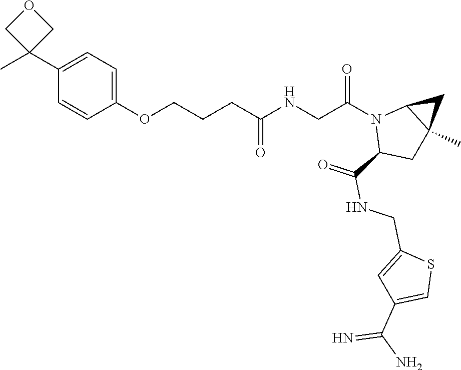 | (1S,3S,5S)-N-((4- carbamimidoylthiophen- 2-yl)methyl)-5-methyl- 2-((4-(4-(3- methyloxetan-3- yl)phenoxy)butanoyl)gl ycyl)-2- azabicyclo[3.1.0]hexane- 3-carboxamide | *** | 1.15 | 568 |
| 141 | 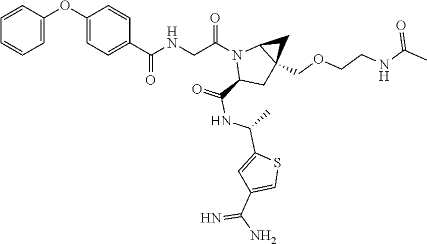 [SMILES: CC(=O)NCCOC[C@@]12C[C@@H]1N(C(=O)CNC(=O)c1ccc(Oc3ccccc3)cc1)[C@H](C(=O)N[C@H](C)c1cc(C(=N)N)cs1)C2] | (1S,3S,5R)-5-((2- acetamidoethoxy)meth yl)-N-((R)-1-(4- carbamimidoylthiophen- 2-yl)ethyl)-2-((4- phenoxybenzoyl)glycyl)-2- azabicyclo[3.1.0]hexane- 3-carboxamide | *** | 1.24 | 647 |
| 142 | 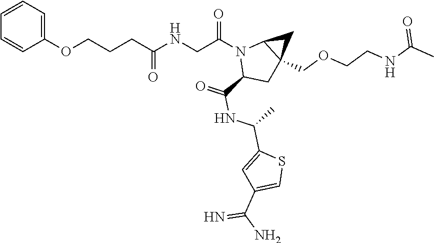 [SMILES: CC(=O)NCCOC[C@@]12C[C@@H]1N(C(=O)CNC(=O)CCCOc1ccccc1)[C@H](C(=O)N[C@H](C)c1cc(C(=N)N)cs1)C2] | (1S,3S,5R)-5-((2- acetamidoethoxy) methyl)-N-((R)-1-(4- carbamimidoylthiophen- 2-yl)ethyl)-2-((4- phenoxybutanoyl)glycyl)-2- azabicyclo[3.1.0]hexane- 3-carboxamide | ** | 0.98 | 613 |
| 143 | 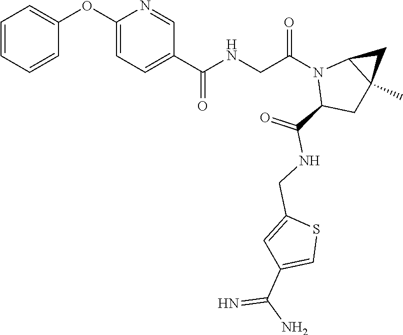 [SMILES: C[C@@]12C[C@@H]1N(C(=O)CNC(=O)c1ccc(Oc3ccccc3)nc1)[C@H](C(=O)NCc1cc(C(=N)N)cs1)C2] | (1S,3S,5S)-N-((4- carbamimidoylthiophen- 2-yl)methyl)-5-methyl- 2-((6- phenoxynicotinoyl) glycyl)-2- azabicyclo[3.1.0]hexane- 3-carboxamide | *** | 1.25 | 533 |
| 144 | 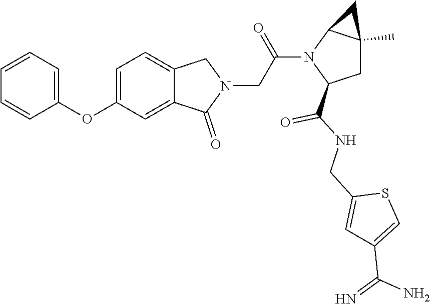 [SMILES: C[C@@]12C[C@@H]1N(C(=O)CN1Cc3ccc(Oc4ccccc4)cc3C1=O)[C@H](C(=O)NCc1cc(C(=N)N)cs1)C2] | (1S,3S,5S)-N-((4- carbamimidoylthiophen- 2-yl)methyl)-5-methyl- 2-(2-(1-oxo-6- phenoxyisoindolin-2- yl)acetyl)-2- azabicyclo[3.1.0]hexane- 3-carboxamide | *** | 1.50 | 544 |
| 145 | 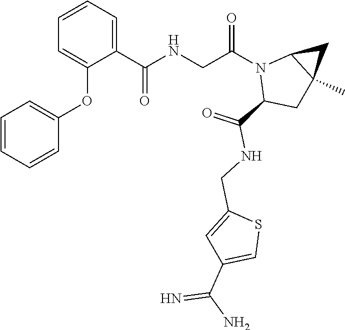 [SMILES: C[C@@]12C[C@@H]1N(C(=O)CNC(=O)c1ccccc1Oc1ccccc1)[C@H](C(=O)NCc1cc(C(=N)N)cs1)C2] | (1S,3S,5S)-N-((4- carbamimidoylthiophen- 2-yl)methyl)-5-methyl- 2-((2- phenoxybenzoyl)glycyl)- 2- azabicyclo[3.1.0]hexane- 3-carboxamide | *** | 1.44 | 532 |
| 147 | 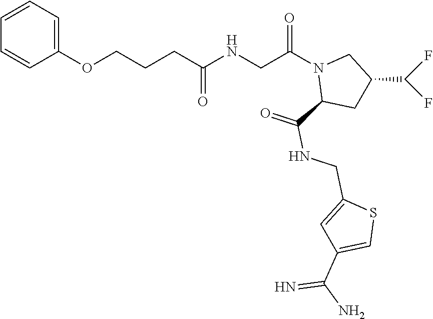 [SMILES: N=C(N)c1csc(CNC(=O)[C@@H]2C[C@@H](C(F)F)CN2C(=O)CNC(=O)CCCOc2ccccc2)c1] | (2S,4R)-N-((4- carbamimidoylthiophen- 2-yl)methyl)-4- (difluoromethyl)-1-((4- phenoxybutanoyl)glycyl) pyrrolidine-2- carboxamide | ** | 1.10 | 522 |
| 148 | 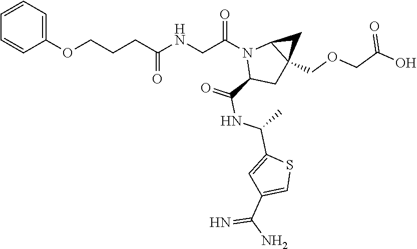 [SMILES: C[C@@H](NC(=O)[C@@H]1C[C@]2(COCC(=O)O)C[C@@H]2N1C(=O)CNC(=O)CCCOc1ccccc1)c1cc(C(=N)N)cs1] | 2-(((1S,3S,5R)-3-(((R)- 1-(4- carbamimidoylthiophen- 2-yl)ethyl)carbamoyl)- 2-((4- phenoxybutanoyl) glycyl)-2- azabicyclo[3.1.0]hexan- 5-yl)methoxy)acetic acid | *** | 1.00 | 586 |
| 149 | 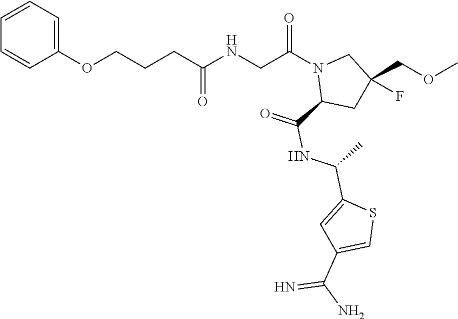 [SMILES: COC[C@@]1(F)C[C@@H](C(=O)N[C@H](C)c2cc(C(=N)N)cs2)N(C(=O)CNC(=O)CCCOc2ccccc2)C1] | (2S,4R)-N-((R)-1-(4- carbamimidoylthiophen- 2-yl)ethyl)-4-fluoro-4- (methoxymethyl)-1-((4- phenoxybutanoyl)glycyl) pyrrolidine-2- carboxamide | *** | 1.06 | 548 |
| 150 | 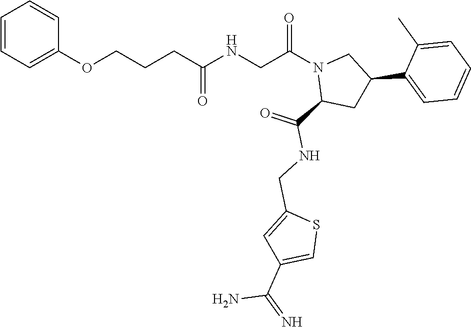 [SMILES: Cc1ccccc1[C@H]1C[C@@H](C(=O)NCc2cc(C(=N)N)cs2)N(C(=O)CNC(=O)CCCOc2ccccc2)C1] | (2S,4R)-N-((4- carbamimidoylthiophen- 2-yl)methyl)-1-((4- phenoxybutanoyl)glycyl)- 4-(o-tolyl)pyrrolidine- 2-carboxamide | *** | 1.48 | 562 |
| 151 | 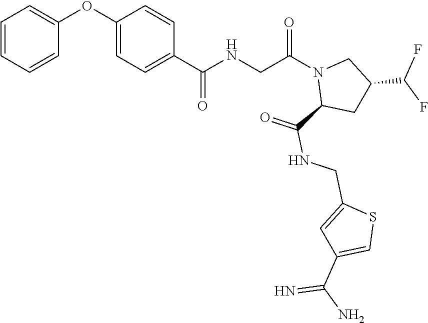 [SMILES: N=C(N)c1csc(CNC(=O)[C@@H]2C[C@@H](C(F)F)CN2C(=O)CNC(=O)c2ccc(Oc3ccccc3)cc2)c1] | (2S,4R)-N-((4- carbamimidoylthiophen- 2-yl)methyl)-4- (difluoromethyl)-1-((4- phenoxybenzoyl)glycyl) pyrrolidine-2- carboxamide | *** | 1.38 | 556 |
| 152 | 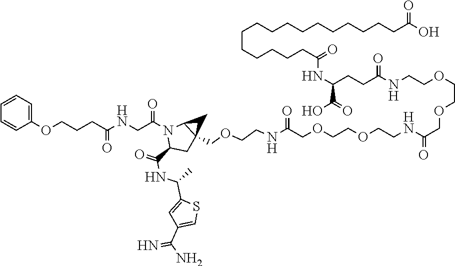 [SMILES: C[C@@H](NC(=O)[C@@H]1C[C@]2(COCCNC(=O)COCCOCCNC(=O)COCCOCCNC(=O)CC[C@H](NC(=O)CCCCCCCCCCCCCCCCC(=O)O)C(=O)O)C[C@@H]2N1C(=O)CNC(=O)CCCOc1ccccc1)c1cc(C(=N)N)cs1] | (S)-1-((1S,3S,5R)-3- (((R)-1-(4- carbamimidoylthiophen- 2-yl)ethyl)carbamoyl)- 2-((4- phenoxybutanoyl)glycyl)-2- azabicyclo[3.1.0]hexan- 5-yl)-27-carboxy- 6,15,24,29-tetraoxo- 2,8,11,17,20-pentaoxa- 5,14,23,28- tetraazahexatetracontan- 46-oic acid | *** | 2.07 | 1286 |
| 153 | 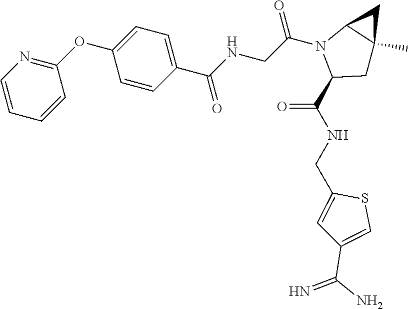 [SMILES: C[C@@]12C[C@@H]1N(C(=O)CNC(=O)c1ccc(Oc3ccccn3)cc1)[C@H](C(=O)NCc1cc(C(=N)N)cs1)C2] | (1S,3S,5S)-N-((4- carbamimidoylthiophen- 2-yl) methyl)-5-methyl- 2-((4-(pyridin-2-yloxy) benzoyl) glycyl)-2- azabicyclo[3.1.0]hexane- 3-carboxamide | *** | 1.14 | 533 |
| 154 | 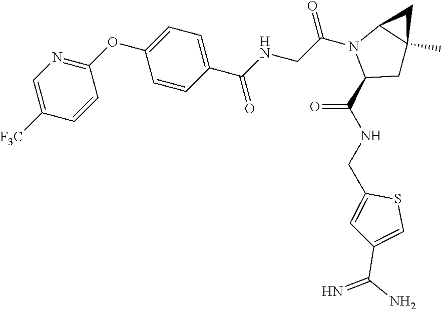 [SMILES: C[C@@]12C[C@@H]1N(C(=O)CNC(=O)c1ccc(Oc3ccc(C(F)(F)F)cn3)cc1)[C@H](C(=O)NCc1cc(C(=N)N)cs1)C2] | (1S,3S,5S)-N-((4- carbamimidoylthiophen- 2-yl)methyl)-5-methyl- 2-((4-((5- (trifluoromethyl)pyridin-2- yl)oxy)benzoyl)glycyl)-2- azabicyclo[3.1.0]hexane- 3-carboxamide | *** | 1.54 | 601 |
| 155 | 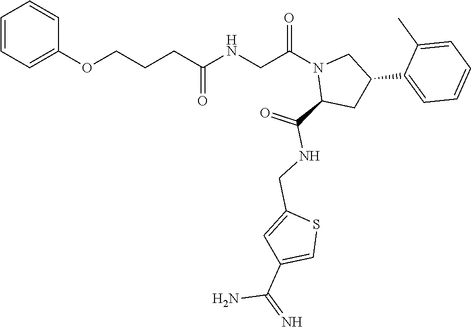 [SMILES: Cc1ccccc1[C@@H]1C[C@@H](C(=O)NCc2cc(C(=N)N)cs2)N(C(=O)CNC(=O)CCCOc2ccccc2)C1] | (2S,4S)-N-((4- carbamimidoylthiophen- 2-yl)methyl)-1-((4- phenoxybutanoyl)glycyl)- 4-(o-tolyl)pyrrolidine- 2-carboxamide | *** | 1.50 | 562 |
| 156 | 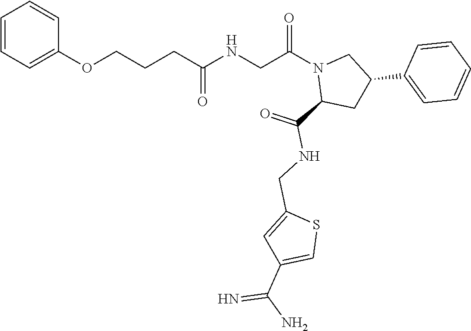 [SMILES: N=C(N)c1csc(CNC(=O)[C@@H]2C[C@@H](c3ccccc3)CN2C(=O)CNC(=O)CCCOc2ccccc2)c1] | (2S,4S)-N-((4- carbamimidoylthiophen- 2-yl)methyl)-1-((4- phenoxybutanoyl)glycyl)- 4-phenylpyrrolidine-2- carboxamide | *** | 1.41 | 548 |
| 157 | 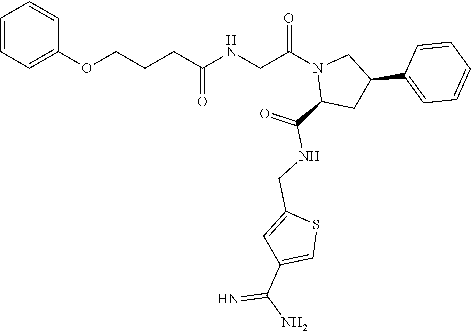 [SMILES: N=C(N)c1csc(CNC(=O)[C@@H]2C[C@H](c3ccccc3)CN2C(=O)CNC(=O)CCCOc2ccccc2)c1] | (2S,4R)-N-((4- carbamimidoylthiophen- 2-yl)methyl)-1-((4- phenoxybutanoyl)glycyl)- 4-phenylpyrrolidine-2- carboxamide | *** | 1.39 | 548 |
| 158 | 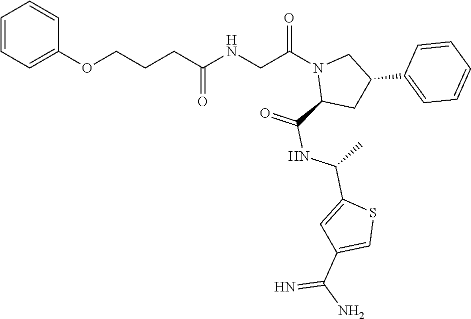 [SMILES: C[C@@H](NC(=O)[C@@H]1C[C@@H](c2ccccc2)CN1C(=O)CNC(=O)CCCOc1ccccc1)c1cc(C(=N)N)cs1] | (2S,4S)-N-((R)-1-(4- carbamimidoylthiophen- 2-yl)ethyl)-1-((4- phenoxybutanoyl)glycyl)- 4-phenylpyrrolidine-2- carboxamide | *** | 1.47 | 562 |
| 159 | 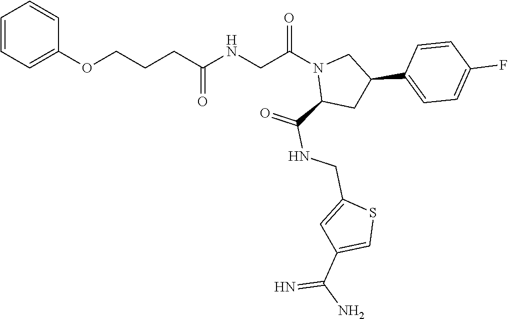 [SMILES: N=C(N)c1csc(CNC(=O)[C@@H]2C[C@H](c3ccc(F)cc3)CN2C(=O)CNC(=O)CCCOc2ccccc2)c1] | (2S,4R)-N-((4- carbamimidoylthiophen- 2-yl)methyl)-4-(4- fluorophenyl)-1-((4- phenoxybutanoyl)glycyl) pyrrolidine-2- carboxamide | *** | 1.43 | 566 |
| 160 | 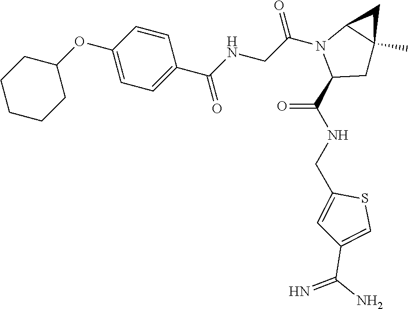 [SMILES: C[C@@]12C[C@@H]1N(C(=O)CNC(=O)c1ccc(OC3CCCCC3)cc1)[C@H](C(=O)NCc1cc(C(=N)N)cs1)C2] | (1S,3S,5S)-N-((4- carbamimidoylthiophen- 2-yl)methyl)-2-((4- (cyclohexyloxy)benzoyl) glycyl)-5-methyl-2- azabicyclo[3.1.0]hexane- 3-carboxamide | *** | 1.63 | 538 |
| 161 | 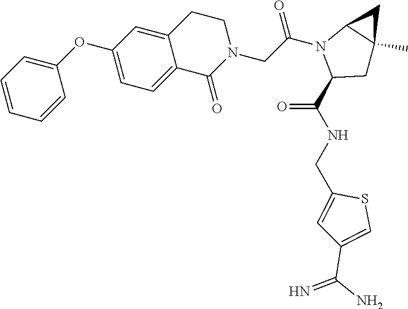 [SMILES: C[C@@]12C[C@@H]1N(C(=O)CN1CCc3cc(Oc4ccccc4)ccc3C1=O)[C@H](C(=O)NCc1cc(C(=N)N)cs1)C2] | (1S,3S,5S)-N-((4- carbamimidoylthiophen- 2-yl)methyl)-5-methyl- 2-(2-(1-oxo-6-phenoxy- 3,4-dihydroisoquinolin- 2(1H)-yl)acetyl)-2- azabicyclo[3.1.0]hexane- 3-carboxamide | *** | 1.60 | 558 |
| 162 | 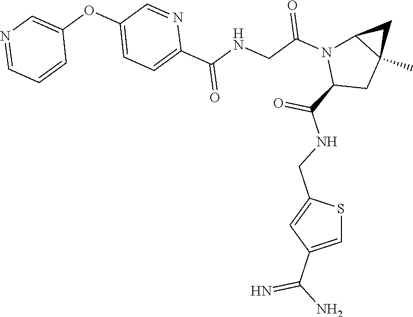 [SMILES: C[C@@]12C[C@@H]1N(C(=O)CNC(=O)c1ccc(Oc3cccnc3)cn1)[C@H](C(=O)NCc1cc(C(=N)N)cs1)C2] | (1S,3S,5S)-N-((4- carbamimidoylthiophen- 2-yl)methyl)-5-methyl- 2-((5-(pyridin-3- yloxy)picolinoyl)glycyl)- 2-azabicyclo[3.1.0]hexane- 3-carboxamide | *** | 0.97 | 534 |
| 164 | 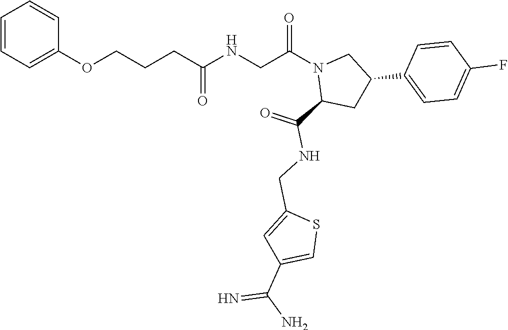 [SMILES: N=C(N)c1csc(CNC(=O)[C@@H]2C[C@@H](c3ccc(F)cc3)CN2C(=O)CNC(=O)CCCOc2ccccc2)c1] | (2S,4S)-N-((4- carbamimidoylthiophen- 2-yl)methyl)-4-(4- fluorophenyl)-1-((4- phenoxybutanoyl)glycyl) pyrrolidine-2- carboxamide | *** | 1.55 | 566 |
| 165 | 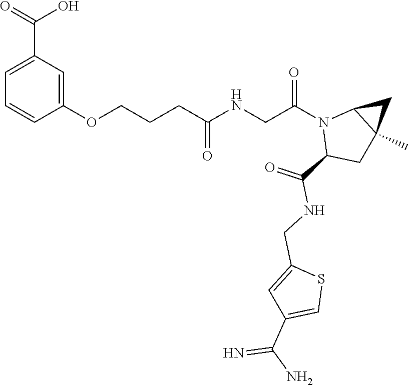 [SMILES: C[C@@]12C[C@@H]1N(C(=O)CNC(=O)CCCOc1cccc(C(=O)O)c1)[C@H](C(=O)NCc1cc(C(=N)N)cs1)C2] | 3-(4-((2-((1S,3S,5S)-3- (((4- carbamimidoylthiophen-2- yl)methyl)carbamoyl)- 5-methyl-2- azabicyclo[3.1.0]hexan- 2-yl)-2- oxoethyl)amino)-4- oxobutoxy)benzoic acid | *** | 0.95 | 542 |
| 166 | 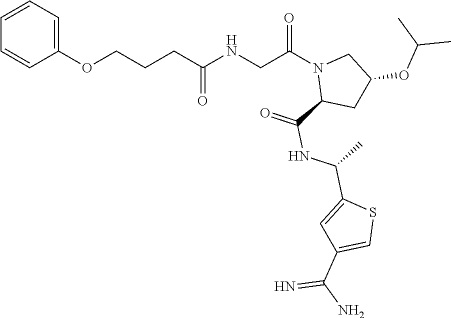 [SMILES: CC(C)O[C@@H]1C[C@@H](C(=O)N[C@H](C)c2cc(C(=N)N)cs2)N(C(=O)CNC(=O)CCCOc2ccccc2)C1] | (2S,4R)-N-((R)-1-(4- carbamimidoylthiophen- 2-yl)ethyl)-4- isopropoxy-1-((4- phenoxybutanoyl)glycyl) pyrrolidine-2- carboxamide | *** | 1.24 | 544 |
| 167 | 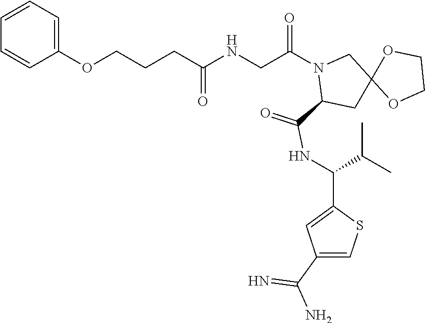 [SMILES: CC(C)[C@@H](NC(=O)[C@@H]1CC2(CN1C(=O)CNC(=O)CCCOc1ccccc1)OCCO2)c1cc(C(=N)N)cs1] | (S)-N-((R)-1-(4- carbamimidoylthiophen- 2-yl)-2-methylpropyl)- 7-((4- phenoxybutanoyl)glycyl)- 1,4-dioxa-7- azaspiro[4.4]nonane-8- carboxamide | * | 1.25 | 572 |
| 168 | 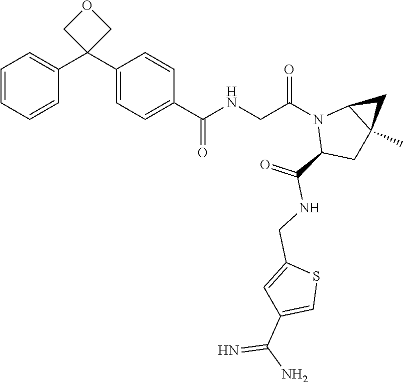 [SMILES: C[C@@]12C[C@@H]1N(C(=O)CNC(=O)c1ccc(C3(c4ccccc4)COC3)cc1)[C@H](C(=O)NCc1cc(C(=N)N)cs1)C2] | (1S,3S,5S)-N-((4- carbamimidoylthiophen- 2-yl)methyl)-5-methyl- 2-((4-(3-phenyloxetan- 3-yl)benzoyl)glycyl)-2- azabicyclo[3.1.0]hexane- 3-carboxamide | *** | 1.39 | 572 |
| 169 | 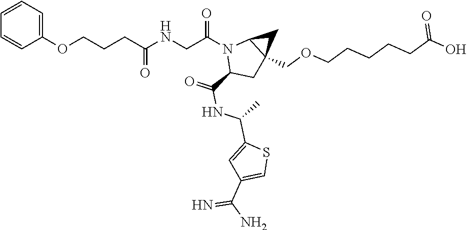 [SMILES: C[C@@H](NC(=O)[C@@H]1C[C@]2(COCCCCCC(=O)O)C[C@@H]2N1C(=O)CNC(=O)CCCOc1ccccc1)c1cc(C(=N)N)cs1] | 6-(((1S,3S,5R)-3-(((R)- 1-(4- carbamimidoylthiophen- 2-yl)ethyl)carbamoyl)- 2-((4- phenoxybutanoyl) glycyl)-2- azabicyclo[3.1.0]hexan- 5-yl)methoxy)hexanoic acid | *** | 1.33 | 642 |
| 170 | 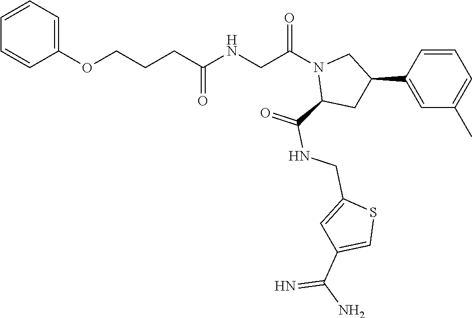 [SMILES: Cc1cccc([C@H]2C[C@@H](C(=O)NCc3cc(C(=N)N)cs3)N(C(=O)CNC(=O)CCCOc3ccccc3)C2)c1] | (2S,4R)-N-((4- carbamimidoylthiophen- 2-yl)methyl)-1-((4- phenoxybutanoyl)glycyl)- 4-(m-tolyl)pyrrolidine- 2-carboxamide | *** | 1.56 | 562 |
| 171 | 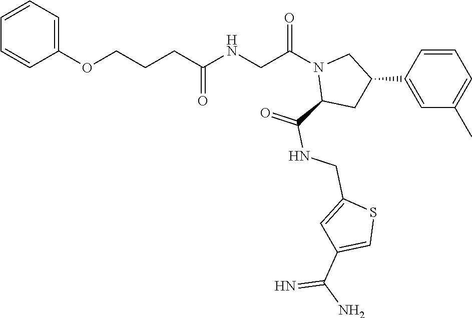 [SMILES: Cc1cccc([C@@H]2C[C@@H](C(=O)NCc3cc(C(=N)N)cs3)N(C(=O)CNC(=O)CCCOc3ccccc3)C2)c1] | (2S,4S)-N-((4- carbamimidoylthiophen- 2-yl)methyl)-1-((4- phenoxybutanoyl)glycyl)- 4-(m-tolyl)pyrrolidine- 2-carboxamide | *** | 1.58 | 562 |
| 172 | 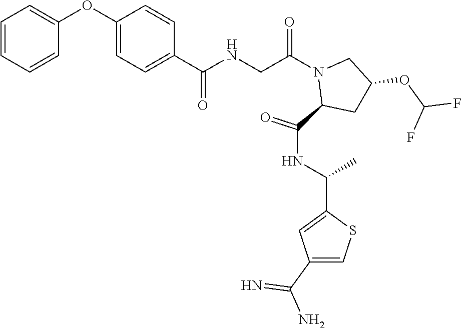 [SMILES: C[C@@H](NC(=O)[C@@H]1C[C@@H](OC(F)F)CN1C(=O)CNC(=O)c1ccc(Oc2ccccc2)cc1)c1cc(C(=N)N)cs1] | (2S,4R)-N-((R)-1-(4- carbamimidoylthiophen- 2-yl)ethyl)-4- (difluoromethoxy)-1- ((4- phenoxybenzoyl)glycyl) pyrrolidine-2- carboxamide | *** | 1.48 | 586 |
| 173 | 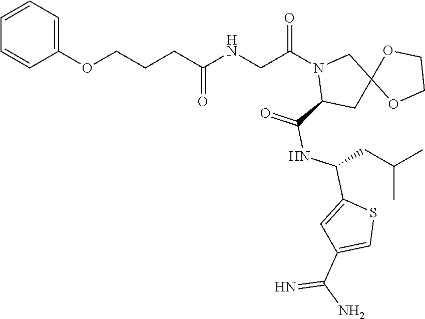 [SMILES: CC(C)C[C@@H](NC(=O)[C@@H]1CC2(CN1C(=O)CNC(=O)CCCOc1ccccc1)OCCO2)c1cc(C(=N)N)cs1] | (S)-N-((R)-1-(4- carbamimidoylthiophen- 2-yl)-3-methylbutyl)-7- ((4- phenoxybutanoyl)glycyl)- 1,4-dioxa-7- azaspiro[4.4]nonane-8- carboxamide | * | 1.38 | 586 |
| 174 | 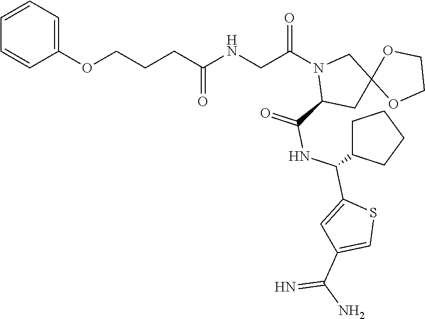 [SMILES: N=C(N)c1csc([C@H](NC(=O)[C@@H]2CC3(CN2C(=O)CNC(=O)CCCOc2ccccc2)OCCO3)C2CCCC2)c1] | (S)-N-((R)-(4- carbamimidoylthiophen-2- yl)(cyclopentyl)methyl)- 7-((4- phenoxybutanoyl)glycyl)- 1,4-dioxa-7- azaspiro[4.4]nonane-8- carboxamide | * | 1.40 | 598 |
| 175 | 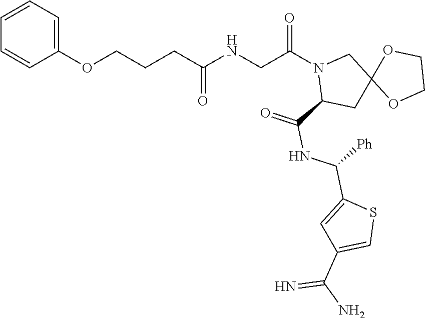 [SMILES: N=C(N)c1csc([C@H](NC(=O)[C@@H]2CC3(CN2C(=O)CNC(=O)CCCOc2ccccc2)OCCO3)c2ccccc2)c1] | (S)-N-((R)-(4- carbamimidoylthiophen- 2-yl)(phenyl)methyl)-7- ((4- phenoxybutanoyl)glycyl)- 1,4-dioxa-7- azaspiro[4.4]nonane-8- carboxamide | * | 1.41 | 606 |
| 176 | 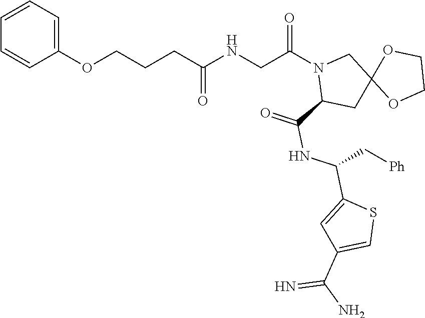 [SMILES: N=C(N)c1csc([C@@H](Cc2ccccc2)NC(=O)[C@@H]2CC3(CN2C(=O)CNC(=O)CCCOc2ccccc2)OCCO3)c1] | (S)-N-((R)-1-(4- carbamimidoylthiophen- 2-yl)-2-phenylethyl)-7- ((4- phenoxybutanoyl)glycyl)- 1,4-dioxa-7- azaspiro[4.4]nonane-8- carboxamide | * | 1.41 | 620 |
| 177 | 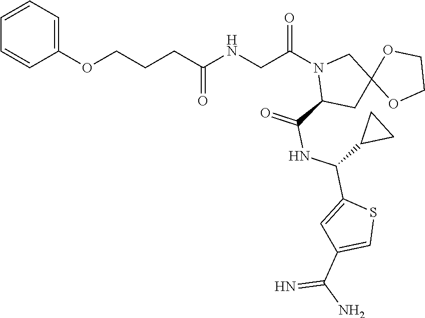 [SMILES: N=C(N)c1csc([C@H](NC(=O)[C@@H]2CC3(CN2C(=O)CNC(=O)CCCOc2ccccc2)OCCO3)C2CC2)c1] | (S)-N-((R)-(4- carbamimidoylthiophen-2- yl)(cyclopropyl)methyl)- 7-((4- phenoxybutanoyl)glycyl)- 1,4-dioxa-7- azaspiro[4.4]nonane-8- carboxamide | * | 1.17 | 570 |
| 178 | 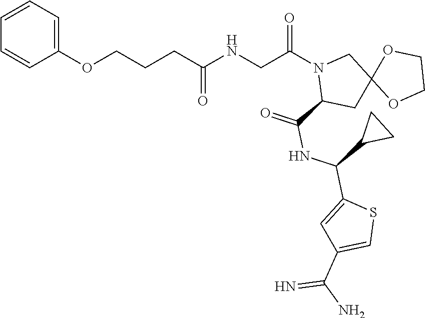 [SMILES: N=C(N)c1csc([C@@H](NC(=O)[C@@H]2CC3(CN2C(=O)CNC(=O)CCCOc2ccccc2)OCCO3)C2CC2)c1] | (S)-N-((S)-(4- carbamimidoylthiophen-2- yl)(cyclopropyl)methyl)- 7-((4- phenoxybutanoyl)glycyl)- 1,4-dioxa-7- azaspiro[4.4]nonane-8- carboxamide | * | 1.23 | 570 |
| 179 | 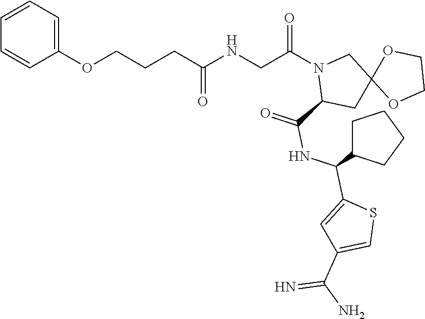 [SMILES: N=C(N)c1csc([C@@H](NC(=O)[C@@H]2CC3(CN2C(=O)CNC(=O)CCCOc2ccccc2)OCCO3)C2CCCC2)c1] | (S)-N-((S)-(4- carbamimidoylthiophen-2- yl)(cyclopentyl)methyl)- 7-((4- phenoxybutanoyl)glycyl)- 1,4-dioxa-7- azaspiro[4.4]nonane-8- carboxamide | * | | 598 |
| 180 | 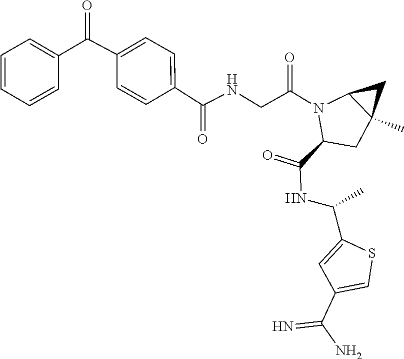 [SMILES: C[C@@H](NC(=O)[C@@H]1C[C@]2(C)C[C@@H]2N1C(=O)CNC(=O)c1ccc(C(=O)c2ccccc2)cc1)c1cc(C(=N)N)cs1] | (1S,3S,5S)-2-((4- benzoylbenzoyl)glycyl)- N-((R)-1-(4- carbamimidoylthiophen- 2-yl)ethyl)-5-methyl-2- azabicyclo[3.1.0]hexane- 3-carboxamide | *** | 1.33 | 558 |
| 181 | 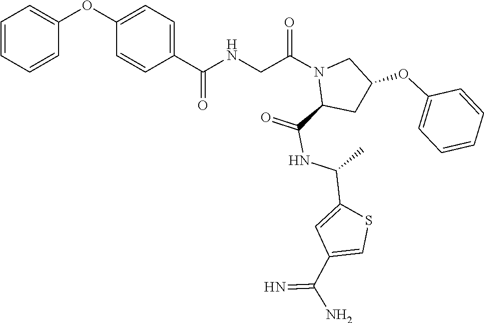 [SMILES: C[C@@H](NC(=O)[C@@H]1C[C@@H](Oc2ccccc2)CN1C(=O)CNC(=O)c1ccc(Oc2ccccc2)cc1)c1cc(C(=N)N)cs1] | (2S,4R)-N-((R)-1-(4- carbamimidoylthiophen- 2-yl)ethyl)-4-phenoxy- 1-((4- phenoxybenzoyl)glycyl) pyrrolidine-2- carboxamide | *** | 1.70 | 612 |
| 182 | 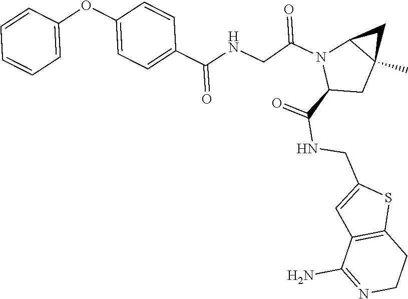 [SMILES: C[C@@]12C[C@@H]1N(C(=O)CNC(=O)c1ccc(Oc3ccccc3)cc1)[C@H](C(=O)NCc1cc3c(s1)CCN=C3N)C2] | (1S,3S,5S)-N-((4- amino-6,7- dihydrothieno[3,2- c]pyridin-2-yl)methyl)- 5-methyl-2-((4- phenoxybenzoyl)glycyl)- 2- azabicyclo[3.1.0]hexane- 3-carboxamide | * | 1.52 | 558 |
| 183 | 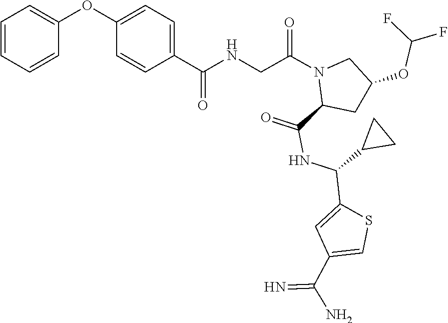 [SMILES: N=C(N)c1csc([C@H](NC(=O)[C@@H]2C[C@@H](OC(F)F)CN2C(=O)CNC(=O)c2ccc(Oc3ccccc3)cc2)C2CC2)c1] | (2S,4R)-N-((R)-(4- carbamimidoylthiophen-2- yl)(cyclopropyl)methyl)- 4-(difluoromethoxy)-1- ((4- phenoxybenzoyl)glycyl) pyrrolidine-2- carboxamide | * | 1.55 | 612 |
| 184 | 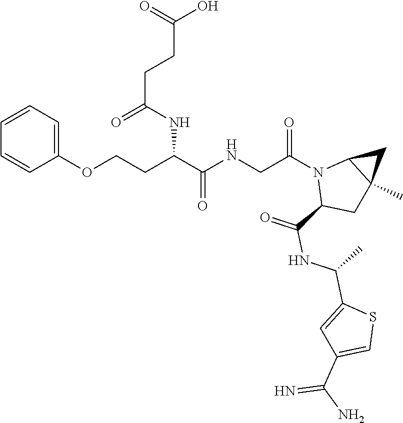 [SMILES: C[C@@H](NC(=O)[C@@H]1C[C@]2(C)C[C@@H]2N1C(=O)CNC(=O)[C@H](CCOc1ccccc1)NC(=O)CCC(=O)O)c1cc(C(=N)N)cs1] | 4-(((S)-1-((2- ((1S,3S,5S)-3-(((R)-1- (4- carbamimidoylthiophen- 2-yl)ethyl)carbamoyl)- 5-methyl-2- azabicyclo[3.1.0]hexan- 2-yl)-2- oxoethyl)amino)-1-oxo- 4-phenoxybutan-2- yl)amino)-4- oxobutanoic acid | *** | 1.10 | 627 |
| 185 | 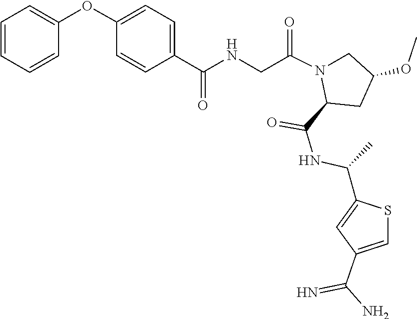 [SMILES: CO[C@@H]1C[C@@H](C(=O)N[C@H](C)c2cc(C(=N)N)cs2)N(C(=O)CNC(=O)c2ccc(Oc3ccccc3)cc2)C1] | (2S,4R)-N-((R)-1-(4- carbamimidoylthiophen- 2-yl)ethyl)-4-methoxy- 1-((4- phenoxybenzoyl)glycyl) pyrrolidine-2- carboxamide | *** | 1.25 | 550 |
| 186 | 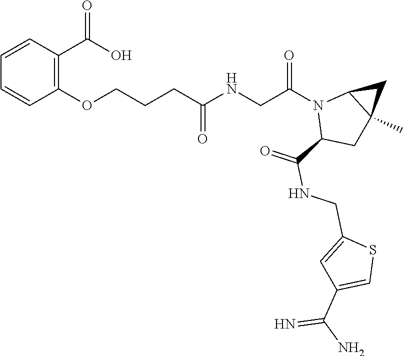 [SMILES: C[C@@]12C[C@@H]1N(C(=O)CNC(=O)CCCOc1ccccc1C(=O)O)[C@H](C(=O)NCc1cc(C(=N)N)cs1)C2] | 2-(4-((2-((1S,3S,5S)-3- (((4- carbamimidoylthiophen- 2- yl)methyl)carbamoyl)- 5-methyl-2- azabicyclo[3.1.0]hexan- 2-yl)-2- oxoethyl)amino)-4- oxobutoxy)benzoic acid | *** | 0.93 | 542 |
| 187 | 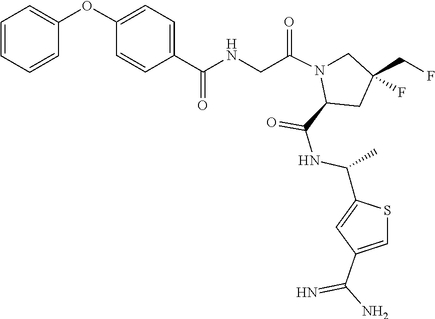 [SMILES: C[C@@H](NC(=O)[C@@H]1C[C@](F)(CF)CN1C(=O)CNC(=O)c1ccc(Oc2ccccc2)cc1)c1cc(C(=N)N)cs1] | (2S,4R)-N-((R)-1-(4- carbamimidoylthiophen- 2-yl)ethyl)-4-fluoro-4- (fluoromethyl)-1-((4- phenoxybenzoyl)glycyl) pyrrolidine-2- carboxamide | *** | 1.38 | 570 |
| 188 | 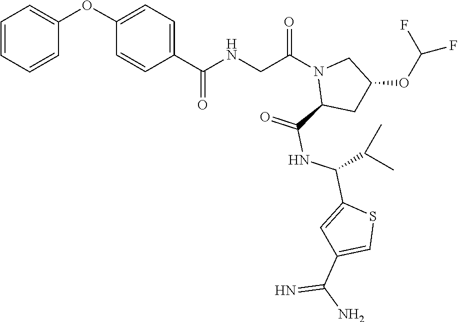 [SMILES: CC(C)[C@@H](NC(=O)[C@@H]1C[C@@H](OC(F)F)CN1C(=O)CNC(=O)c1ccc(Oc2ccccc2)cc1)c1cc(C(=N)N)cs1] | (2S,4R)-N-((R)-1-(4- carbamimidoylthiophen- 2-yl)-2-methylpropyl)- 4-(difluoromethoxy)-1- ((4- phenoxybenzoyl)glycyl) pyrrolidine-2- carboxamide | * | 1.57 | 614 |
| 189 | 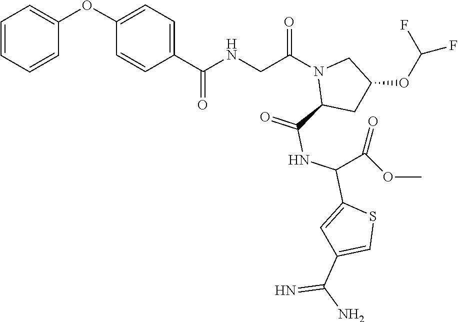 [SMILES: COC(=O)C(NC(=O)[C@@H]1C[C@@H](OC(F)F)CN1C(=O)CNC(=O)c1ccc(Oc2ccccc2)cc1)c1cc(C(=N)N)cs1] | methyl 2-(4- carbamimidoylthiophen- 2-yl)-2-((2S,4R)-4- (difluoromethoxy)-1- ((4- phenoxybenzoyl)glycyl) pyrrolidine-2- carboxamido)acetate | * | 1.48 | 630 |
| 190 | 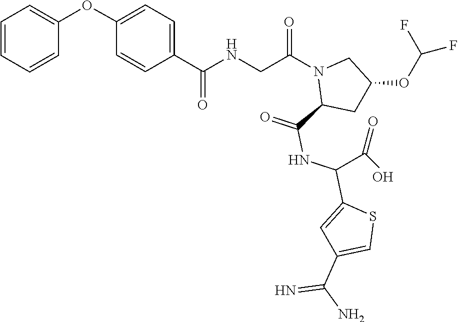 [SMILES: N=C(N)c1csc(C(NC(=O)[C@@H]2C[C@@H](OC(F)F)CN2C(=O)CNC(=O)c2ccc(Oc3ccccc3)cc2)C(=O)O)c1] | 2-(4- carbamimidoylthiophen- 2-yl)-2-((2S,4R)-4- (difluoromethoxy)-1- ((4- phenoxybenzoyl)glycyl) pyrrolidine-2- carboxamido)acetic acid | *** | 1.60 | 617 |
| 191 | 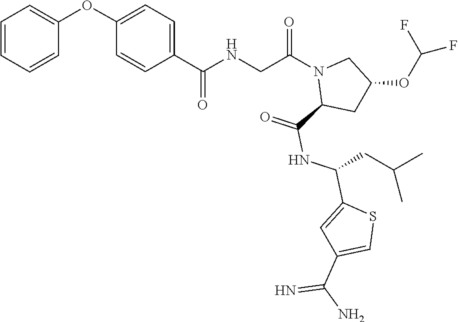 [SMILES: CC(C)C[C@@H](NC(=O)[C@@H]1C[C@@H](OC(F)F)CN1C(=O)CNC(=O)c1ccc(Oc2ccccc2)cc1)c1cc(C(=N)N)cs1] | (2S,4R)-N-((R)-1-(4- carbamimidoylthiophen- 2-yl)-3-methylbutyl)-4- (difluoromethoxy)-1- ((4- phenoxybenzoyl)glycyl) pyrrolidine-2- carboxamide | * | 1.72 | 628 |
| 192 | 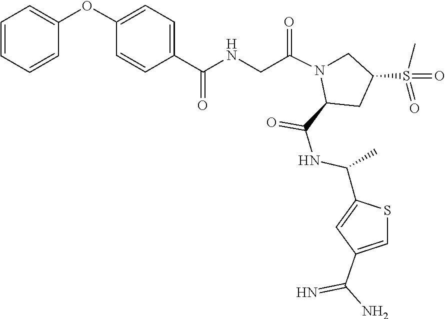 [SMILES: C[C@@H](NC(=O)[C@@H]1C[C@@H](S(C)(=O)=O)CN1C(=O)CNC(=O)c1ccc(Oc2ccccc2)cc1)c1cc(C(=N)N)cs1] | (2S,4R)-N-((R)-1-(4- carbamimidoylthiophen- 2-yl)ethyl)-4- (methylsulfonyl)-1-((4- phenoxybenzoyl)glycyl) pyrrolidine-2- carboxamide | *** | 1.25 | 598 |
| 193 | 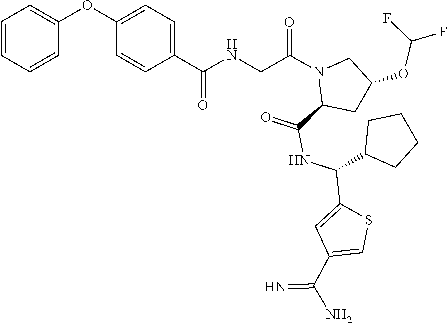 [SMILES: N=C(N)c1csc([C@H](NC(=O)[C@@H]2C[C@@H](OC(F)F)CN2C(=O)CNC(=O)c2ccc(Oc3ccccc3)cc2)C2CCCC2)c1] | (2S,4R)-N-((R)-(4- carbamimidoylthiophen-2- yl)(cyclopentyl)methyl)- 4-(difluoromethoxy)-1- ((4- phenoxybenzoyl)glycyl) pyrrolidine-2- carboxamide | * | 1.76 | 640 |
| 194 | 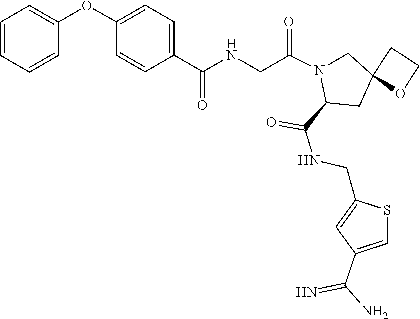 [SMILES: N=C(N)c1csc(CNC(=O)[C@@H]2C[C@@]3(CCO3)CN2C(=O)CNC(=O)c2ccc(Oc3ccccc3)cc2)c1] | (4R,7S)-N-((4- carbamimidoylthiophen- 2-yl)methyl)-6-((4- phenoxybenzoyl)glycyl)- 1-oxa-6- azaspiro[3.4]octane-7- carboxamide | *** | 1.34 | 548 |
| 195 | 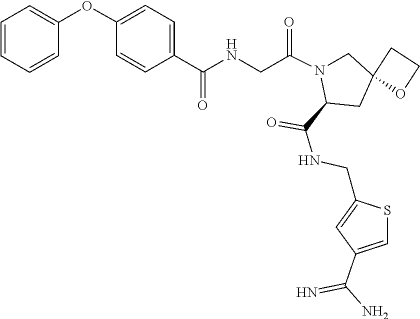 [SMILES: N=C(N)c1csc(CNC(=O)[C@@H]2C[C@]3(CCO3)CN2C(=O)CNC(=O)c2ccc(Oc3ccccc3)cc2)c1] | (4S,7S)-N-((4- carbamimidoylthiophen- 2-yl)methyl)-6-((4- phenoxybenzoyl)glycyl)- 1-oxa-6- azaspiro[3.4]octane-7- carboxamide | *** | 1.30 | 548 |
| 196 | 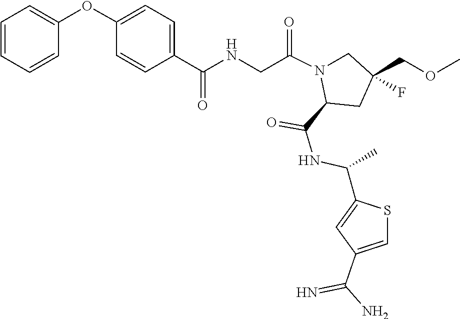 [SMILES: COC[C@@]1(F)C[C@@H](C(=O)N[C@H](C)c2cc(C(=N)N)cs2)N(C(=O)CNC(=O)c2ccc(Oc3ccccc3)cc2)C1] | (2S,4R)-N-((R)-1-(4- carbamimidoylthiophen- 2-yl)ethyl)-4-fluoro-4- (methoxymethyl)-1-((4- phenoxybenzoyl)glycyl) pyrrolidine-2- carboxamide | *** | 1.39 | 582 |
| 197 | 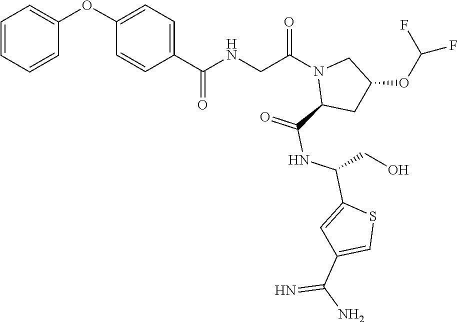 [SMILES: N=C(N)c1csc([C@@H](CO)NC(=O)[C@@H]2C[C@@H](OC(F)F)CN2C(=O)CNC(=O)c2ccc(Oc3ccccc3)cc2)c1] | (2S,4R)-N-((R)-1-(4- carbamimidoylthiophen- 2-yl)-2-hydroxyethyl)- 4-(difluoromethoxy)-1- ((4- phenoxybenzoyl)glycyl) pyrrolidine-2- carboxamide | *** | 1.39 | 602 |
| 198 | 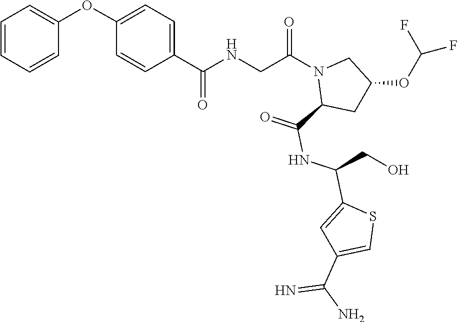 [SMILES: N=C(N)c1csc([C@H](CO)NC(=O)[C@@H]2C[C@@H](OC(F)F)CN2C(=O)CNC(=O)c2ccc(Oc3ccccc3)cc2)c1] | (2S,4R)-N-((S)-1-(4- carbamimidoylthiophen- 2-yl)-2-hydroxyethyl)- 4-(difluoromethoxy)-1- ((4- phenoxybenzoyl)glycyl) pyrrolidine-2- carboxamide | *** | 1.39 | 602 |
| 199 | 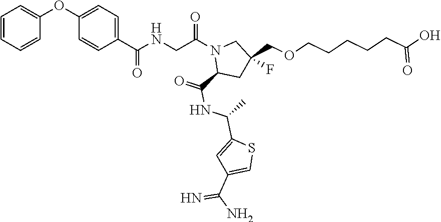 [SMILES: C[C@@H](NC(=O)[C@@H]1C[C@](F)(COCCCCCC(=O)O)CN1C(=O)CNC(=O)c1ccc(Oc2ccccc2)cc1)c1cc(C(=N)N)cs1] | 6-(((3R,5S)-5-(((R)-1- (4- carbamimidoylthiophen- 2-yl)ethyl)carbamoyl)- 3-fluoro-1-((4- phenoxybenzoyl)glycyl) pyrrolidin-3- yl) methoxy)hexanoic acid | *** | 1.55 | 682 |
| 200 | 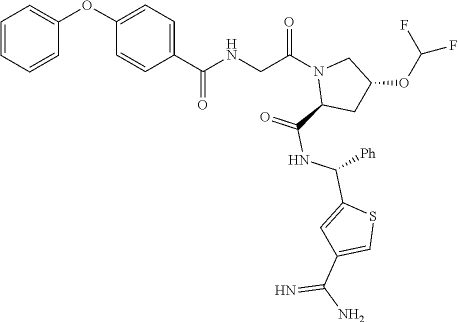 [SMILES: N=C(N)c1csc([C@H](NC(=O)[C@@H]2C[C@@H](OC(F)F)CN2C(=O)CNC(=O)c2ccc(Oc3ccccc3)cc2)c2ccccc2)c1] | (2S,4R)-N-((R)-(4- carbamimidoylthiophen- 2-yl)(phenyl)methyl)-4- (difluoromethoxy)-1- ((4- phenoxybenzoyl)glycyl) pyrrolidine-2- carboxamide | * | 1.72 | 648 |
| 201 | 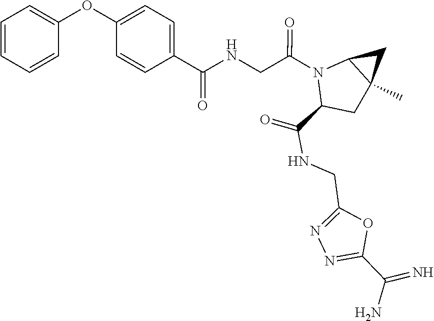 [SMILES: C[C@@]12C[C@@H]1N(C(=O)CNC(=O)c1ccc(Oc3ccccc3)cc1)[C@H](C(=O)NCc1nnc(C(=N)N)o1)C2] | (1S,3S,5S)-N-((5- carbamimidoyl-1,3,4- oxadiazol-2-yl)methyl)- 5-methyl-2-((4- phenoxybenzoyl)glycyl)-2- azabicyclo[3.1.0]hexane- 3-carboxamide | * | 1.36 | 518 |
| 202 | 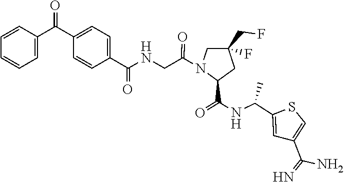 [SMILES: C[C@@H](NC(=O)[C@@H]1C[C@](F)(CF)CN1C(=O)CNC(=O)c1ccc(C(=O)c2ccccc2)cc1)c1cc(C(=N)N)cs1] | (2S,4R)-1-((4- benzoylbenzoyl)glycyl)- N-((R)-1-(4- carbamimidoylthiophen- 2-yl)ethyl)-4-fluoro-4- (fluoromethyl)pyrrolidine- 2-carboxamide | *** | 1.28 | 582 |
| 203 | 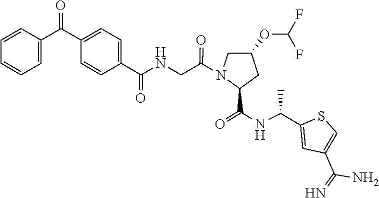 [SMILES: C[C@@H](NC(=O)[C@@H]1C[C@@H](OC(F)F)CN1C(=O)CNC(=O)c1ccc(C(=O)c2ccccc2)cc1)c1cc(C(=N)N)cs1] | (2S,4R)-1-((4- benzoylbenzoyl)glycyl)- N-((R)-1-(4- carbamimidoylthiophen- 2-yl)ethyl)-4- (difluoromethoxy) pyrrolidine-2-carboxamide | *** | 1.34 | 598 |
| 204 | 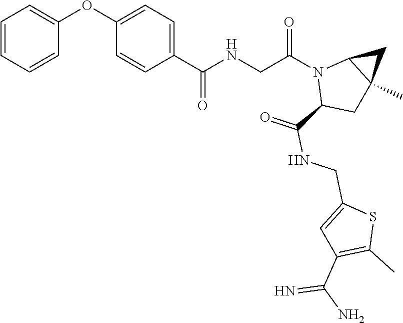 [SMILES: Cc1sc(CNC(=O)[C@@H]2C[C@]3(C)C[C@@H]3N2C(=O)CNC(=O)c2ccc(Oc3ccccc3)cc2)cc1C(=N)N] | (1S,3S,5S)-N-((4- carbamimidoyl-5- methylthiophen-2- yl)methyl)-5-methyl-2-((4- phenoxybenzoyl)glycyl)-2- azabicyclo[3.1.0]hexane- 3-carboxamide | * | 1.52 | 546 |
| 205 | 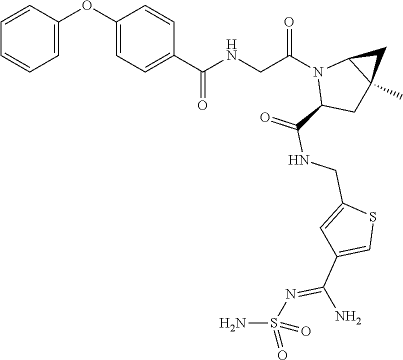 [SMILES: C[C@@]12C[C@@H]1N(C(=O)CNC(=O)c1ccc(Oc3ccccc3)cc1)[C@H](C(=O)NCc1cc(/C(N)=N/S(N)(=O)=O)cs1)C2] | (1S,3S,5S)-5-methyl-2- ((4- phenoxybenzoyl)glycyl)- N-((4-((Z)-N′- sulfamoylcarbamimidoyl) thiophen-2-yl)methyl)-2- azabicyclo[3.1.0]hexane- 3-carboxamide | * | 2.03 | 611 |
| 206 | 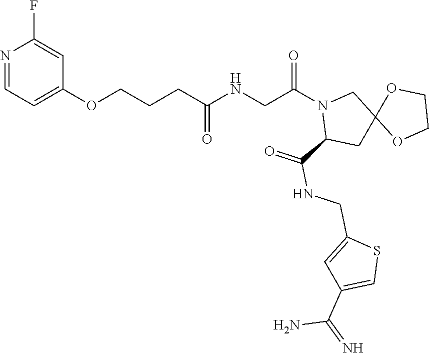 [SMILES: N=C(N)c1csc(CNC(=O)[C@@H]2CC3(CN2C(=O)CNC(=O)CCCOc2ccnc(F)c2)OCCO3)c1] | (S)-N-((4- carbamimidoylthiophen- 2-yl)methyl)-7-((4-((2- fluoropyridin-4- yl)oxy)butanoyl)glycyl)- 1,4-dioxa-7- azaspiro[4.4]nonane-8- carboxamide | *** | 0.61 | 549 |
| 207 | 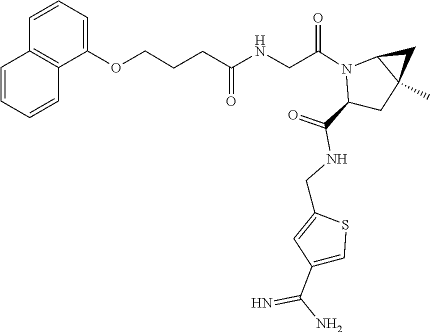 [SMILES: C[C@@]12C[C@@H]1N(C(=O)CNC(=O)CCCOc1cccc3ccccc13)[C@H](C(=O)NCc1cc(C(=N)N)cs1)C2] | (1S,3S,5S)-N-((4- carbamimidoylthiophen- 2-yl)methyl)-5-methyl- 2-((4-(naphthalen-1- yloxy)butanoyl)glycyl)-2- azabicyclo[3.1.0]hexane- 3-carboxamide | *** | 1.61 | 548 |
| 208 | 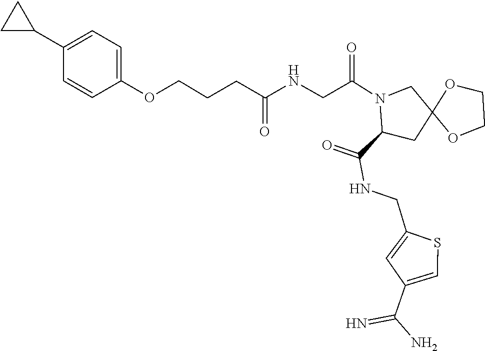 [SMILES: N=C(N)c1csc(CNC(=O)[C@@H]2CC3(CN2C(=O)CNC(=O)CCCOc2ccc(C4CC4)cc2)OCCO3)c1] | (S)-N-((4- carbamimidoylthiophen- 2-yl)methyl)-7-((4-(4- cyclopropylphenoxy) butanoyl)glycyl)-1,4- dioxa-7- azaspiro[4.4]nonane-8- carboxamide | *** | 1.42 | 570 |
| 209 | 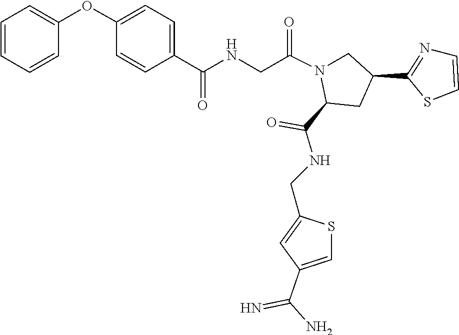 [SMILES: N=C(N)c1csc(CNC(=O)[C@@H]2C[C@H](c3nccs3)CN2C(=O)CNC(=O)c2ccc(Oc3ccccc3)cc2)c1] | (2S,4S)-N-((4- carbamimidoylthiophen- 2-yl)methyl)-1-((4- phenoxybenzoyl)glycyl)- 4-(thiazol-2- yl)pyrrolidine-2- carboxamide | *** | 1.43 | 589 |
| 210 | 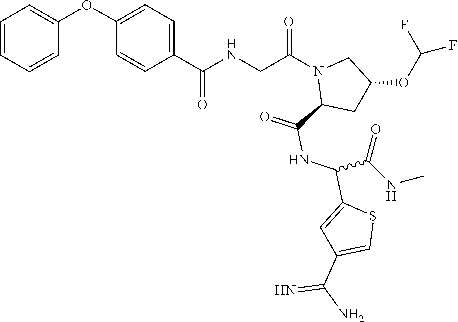 [SMILES: CNC(=O)C(NC(=O)[C@@H]1C[C@@H](OC(F)F)CN1C(=O)CNC(=O)c1ccc(Oc2ccccc2)cc1)c1cc(C(=N)N)cs1] | (2S,4R)-N-(1-(4- carbamimidoylthiophen- 2-yl)-2-(methylamino)- 2-oxoethyl)-4- (difluoromethoxy)-1- ((4- phenoxybenzoyl)glycyl) pyrrolidine-2- carboxamide | * | 1.48 (A) | 629 |
| 211 | 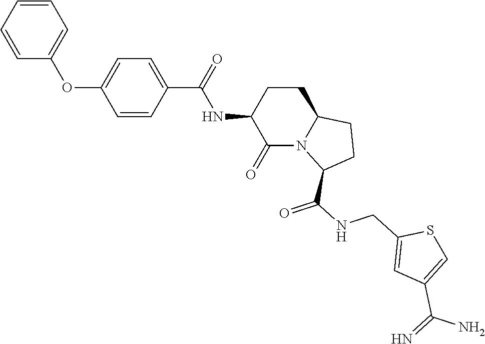 [SMILES: N=C(N)c1csc(CNC(=O)[C@@H]2CC[C@@H]3CC[C@H](NC(=O)c4ccc(Oc5ccccc5)cc4)C(=O)N32)c1] | (3S,6S,8aS)-N-((4- carbamimidoylthiophen- 2-yl)methyl)-5-oxo-6- (4- phenoxybenzamido)oct ahydroindolizine-3- carboxamide | ** | 1.55 | 532 |
| 212 | 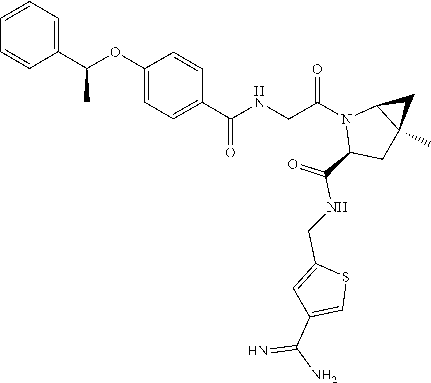 [SMILES: C[C@H](Oc1ccc(C(=O)NCC(=O)N2[C@H]3C[C@@]3(C)C[C@H]2C(=O)NCc2cc(C(=N)N)cs2)cc1)c1ccccc1] | (1S,3S,5S)-N-((4- carbamimidoylthiophen- 2-yl)methyl)-5-methyl- 2-((4-((S)-1- phenylethoxy)benzoyl) glycyl)-2- azabicyclo[3.1.0]hexane- 3-carboxamide | *** | 1.66 | 560 |
| 213 | 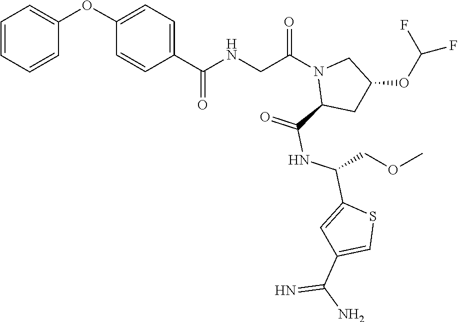 [SMILES: COC[C@@H](NC(=O)[C@@H]1C[C@@H](OC(F)F)CN1C(=O)CNC(=O)c1ccc(Oc2ccccc2)cc1)c1cc(C(=N)N)cs1] | (2S,4R)-N-((R)-1-(4- carbamimidoylthiophen- 2-yl)-2-methoxyethyl)- 4-(difluoromethoxy)-1- ((4- phenoxybenzoyl)glycyl) pyrrolidine-2- carboxamide | * | 1.55 | 616 |
| 214 | 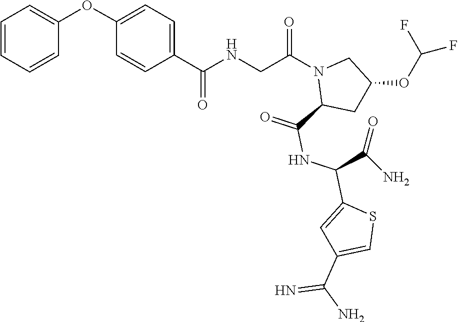 [SMILES: N=C(N)c1csc([C@@H](NC(=O)[C@@H]2C[C@@H](OC(F)F)CN2C(=O)CNC(=O)c2ccc(Oc3ccccc3)cc2)C(N)=O)c1] | (2S,4R)-N-((S)-2- amino-1-(4- carbamimidoylthiophen- 2-yl)-2-oxoethyl)-4- (difluoromethoxy)-1- ((4- phenoxybenzoyl)glycyl) pyrrolidine-2- carboxamide | * | 1.42 | 615 |
| 215 | 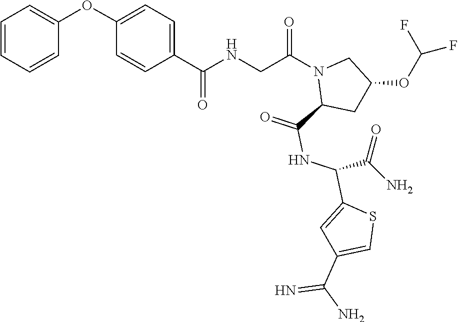 [SMILES: N=C(N)c1csc([C@H](NC(=O)[C@@H]2C[C@@H](OC(F)F)CN2C(=O)CNC(=O)c2ccc(Oc3ccccc3)cc2)C(N)=O)c1] | (2S,4R)-N-((R)-2- amino-1-(4- carbamimidoylthiophen- 2-yl)-2-oxoethyl)-4- (difluoromethoxy)-1- ((4- phenoxybenzoyl)glycyl) pyrrolidine-2- carboxamide | * | 1.44 | 615 |
| 216 | 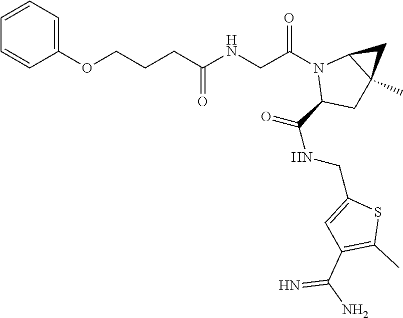 [SMILES: Cc1sc(CNC(=O)[C@@H]2C[C@]3(C)C[C@@H]3N2C(=O)CNC(=O)CCCOc2ccccc2)cc1C(=N)N] | (1S,3S,5S)-N-((4- carbamimidoyl-5- methylthiophen-2- yl)methyl)-5-methyl-2- ((4- phenoxybutanoyl) glycyl)-2- azabicyclo[3.1.0]hexane- 3-carboxamide | * | 1.30 | 512 |
| 217 | 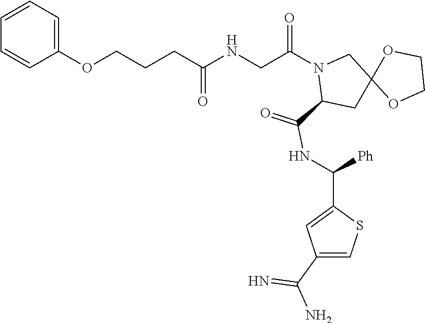 [SMILES: N=C(N)c1csc([C@@H](NC(=O)[C@@H]2CC3(CN2C(=O)CNC(=O)CCCOc2ccccc2)OCCO3)c2ccccc2)c1] | (S)-N-((S)-(4- carbamimidoylthiophen- 2-yl)(phenyl)methyl)-7- ((4- phenoxybutanoyl)glycyl)- 1,4-dioxa-7- azaspiro[4.4]nonane-8- carboxamide | * | 1.41 | 606 |
| 218 | 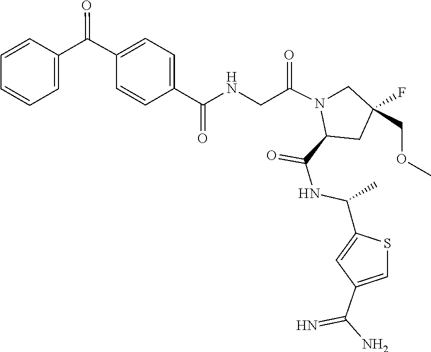 [SMILES: COC[C@@]1(F)C[C@@H](C(=O)N[C@H](C)c2cc(C(=N)N)cs2)N(C(=O)CNC(=O)c2ccc(C(=O)c3ccccc3)cc2)C1] | (2S,4R)-1-((4- benzoylbenzoyl)glycyl)- N-((R)-1-(4- carbamimidoylthiophen- 2-yl)ethyl)-4-fluoro-4- (methoxymethyl) pyrrolidine- 2-carboxamide | *** | 1.28 | 594 |
| 219 | 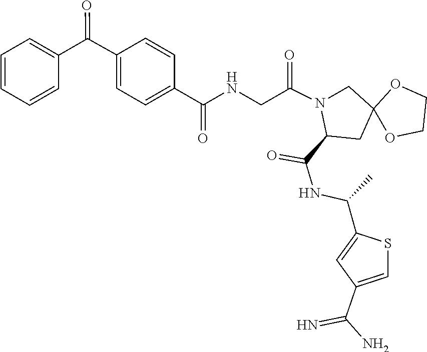 [SMILES: C[C@@H](NC(=O)[C@@H]1CC2(CN1C(=O)CNC(=O)c1ccc(C(=O)c3ccccc3)cc1)OCCO2)c1cc(C(=N)N)cs1] | (S)-7-((4- benzoylbenzoyl)glycyl)- N-((R)-1-(4- carbamimidoylthiophen- 2-yl)ethyl)-1,4-dioxa-7- azaspiro[4.4]nonane-8- carboxamide | *** | 1.26 | 590 |
| 220 | 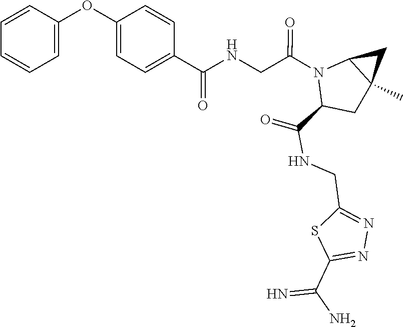 [SMILES: C[C@@]12C[C@@H]1N(C(=O)CNC(=O)c1ccc(Oc3ccccc3)cc1)[C@H](C(=O)NCc1nnc(C(=N)N)s1)C2] | (1S,3S,5S)-N-((5- carbamimidoyl-1,3,4- thiadiazol-2-yl) methyl)- 5-methyl-2-((4- phenoxybenzoyl)glycyl)-2- azabicyclo[3.1.0]hexane- 3-carboxamide | | 1.40 | 534 |
| 221 | 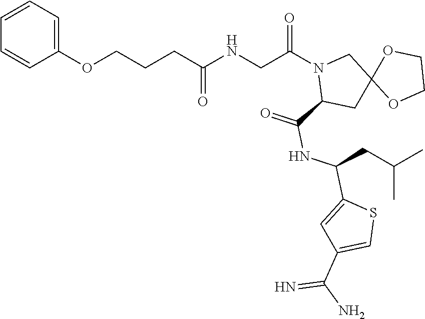 [SMILES: CC(C)C[C@H](NC(=O)[C@@H]1CC2(CN1C(=O)CNC(=O)CCCOc1ccccc1)OCCO2)c1cc(C(=N)N)cs1] | (S)-N-((S)-1-(4- carbamimidoylthiophen- 2-yl)-3-methylbutyl)-7- ((4- phenoxybutanoyl)glycyl)- 1,4-dioxa-7- azaspiro[4.4]nonane-8- carboxamide | * | 1.55 | 586 |
| 222 | 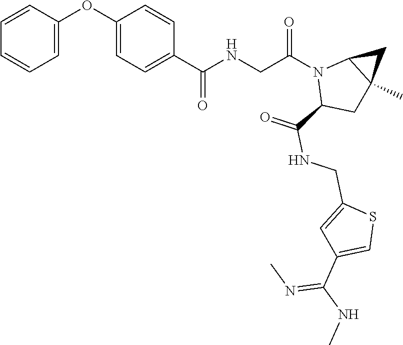 [SMILES: C/N=C(/NC)c1csc(CNC(=O)[C@@H]2C[C@]3(C)C[C@@H]3N2C(=O)CNC(=O)c2ccc(Oc3ccccc3)cc2)c1] | (1S,3S,5S)-N-((4-((E)- N,N′- dimethylcarbamimidoyl) thiophen-2-yl)methyl)- 5-methyl-2-((4- phenoxybenzoyl)glycyl)-2- azabicyclo[3.1.0]hexane- 3-carboxamide | * | 1.57 | 560 |
| 223 | 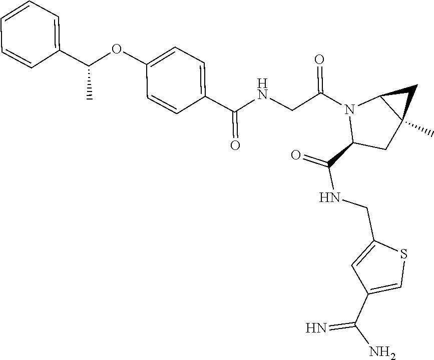 [SMILES: C[C@@H](Oc1ccc(C(=O)NCC(=O)N2[C@H]3C[C@@]3(C)C[C@H]2C(=O)NCc2cc(C(=N)N)cs2)cc1)c1ccccc1] | (1S,3S,5S)-N-((4- carbamimidoylthiophen- 2-yl)methyl)-5-methyl- 2-((4-((R)-1- phenylethoxy)benzoyl) glycyl)-2- azabicyclo[3.1.0]hexane- 3-carboxamide | *** | 1.73 | 560 |
| 234 | 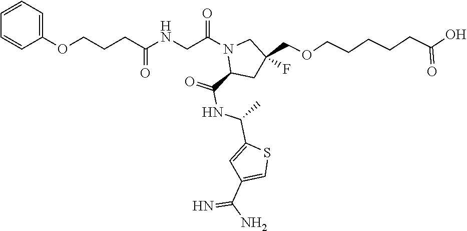 [SMILES: C[C@@H](NC(=O)[C@@H]1C[C@](F)(COCCCCCC(=O)O)CN1C(=O)CNC(=O)CCCOc1ccccc1)c1cc(C(=N)N)cs1] | 6-(((3R,5S)-5-(((R)-1-(4- carbamimidoylthiophen- 2-yl)ethyl)carbamoyl)- 3-fluoro-1-((4- phenoxybutanoyl)glycyl) pyrrolidin-3- yl)methoxy)hexanoic acid | *** | 1.34 | 648 |
| 235 | 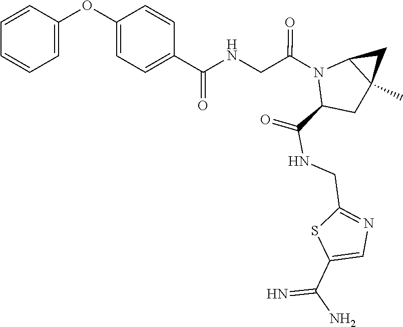 [SMILES: C[C@@]12C[C@@H]1N(C(=O)CNC(=O)c1ccc(Oc3ccccc3)cc1)[C@H](C(=O)NCc1ncc(C(=N)N)s1)C2] | (1S,3S,5S)-N-((5- carbamimidoylthiazol- 2-yl)methyl)-5-methyl- 2-((4- phenoxybenzoyl)glycyl)-2- azabicyclo[3.1.0]hexane- 3-carboxamide | *** | 1.42 | 533 |
| 236 | 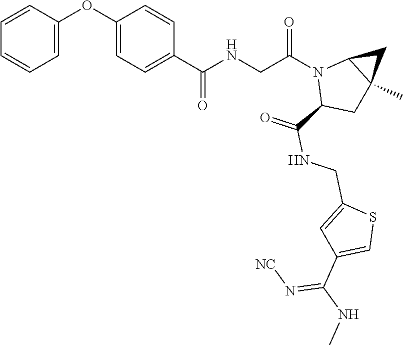 [SMILES: CN/C(=N/C#N)c1csc(CNC(=O)[C@@H]2C[C@]3(C)C[C@@H]3N2C(=O)CNC(=O)c2ccc(Oc3ccccc3)cc2)c1] | (1S,3S,5S)-N-((4-((E)- N′-cyano-N- methylcarbamimidoyl) thiophen-2-yl)methyl)-5- methyl-2-((4- phenoxybenzoyl)glycyl)-2- azabicyclo[3.1.0]hexane- 3-carboxamide | * | 2.23 | 571 |
| 237 | 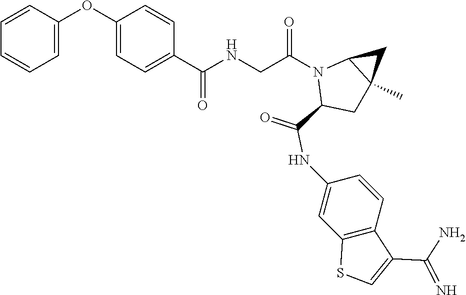 [SMILES: C[C@@]12C[C@@H]1N(C(=O)CNC(=O)c1ccc(Oc3ccccc3)cc1)[C@H](C(=O)Nc1ccc3c(C(=N)N)csc3c1)C2] | (1S,3S,5S)-N-(3- carbamimidoylbenzo[b] thiophen-6-yl)-5- methyl-2-((4- phenoxybenzoyl)glycyl)-2- azabicyclo[3.1.0]hexane- 3-carboxamide | ** | 1.67 | 568 |
| 238 | 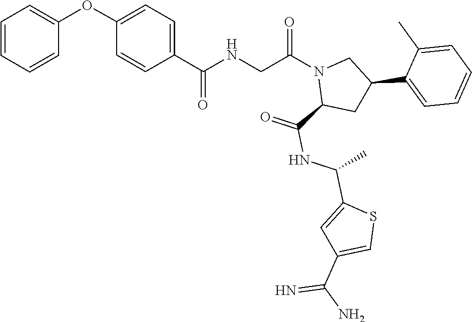 [SMILES: Cc1ccccc1[C@H]1C[C@@H](C(=O)N[C@H](C)c2cc(C(=N)N)cs2)N(C(=O)CNC(=O)c2ccc(Oc3ccccc3)cc2)C1] | (2S,4R)-N-((R)-1-(4- carbamimidoylthiophen- 2-yl)ethyl)-1-((4- phenoxybenzoyl)glycyl)- 4-(o-tolyl)pyrrolidine-2- carboxamide | *** | 1.84 | 610 |
| 239 | 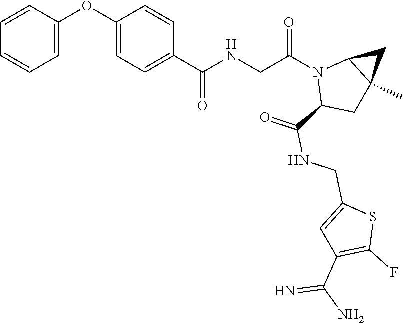 [SMILES: C[C@@]12C[C@@H]1N(C(=O)CNC(=O)c1ccc(Oc3ccccc3)cc1)[C@H](C(=O)NCc1cc(C(=N)N)c(F)s1)C2] | (1S,3S,5S)-N-((4- carbamimidoyl-5- fluorothiophen-2- yl)methyl)-5-methyl-2-((4- phenoxybenzoyl)glycyl)-2- azabicyclo[3.1.0]hexane- 3-carboxamide | *** | 1.44 | 550 |
| 240 | 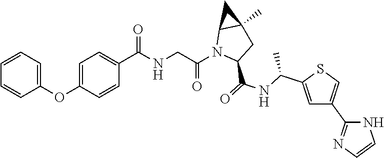 [SMILES: C[C@@H](NC(=O)[C@@H]1C[C@]2(C)C[C@@H]2N1C(=O)CNC(=O)c1ccc(Oc2ccccc2)cc1)c1cc(-c2ncc[nH]2)cs1] | (1S,3S,5S)-N-((R)-1-(4- (1H-imidazol-2- yl)thiophen-2-yl)ethyl)- 5-methyl-2-((4- phenoxybenzoyl)glycyl)-2- azabicyclo[3.1.0]hexane- 3-carboxamide | *** | 1.51 | 570 |
| 241 | 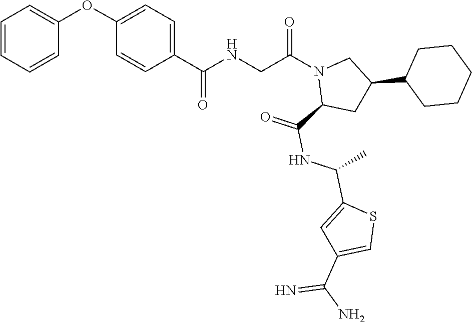 [SMILES: C[C@@H](NC(=O)[C@@H]1C[C@H](C2CCCCC2)CN1C(=O)CNC(=O)c1ccc(Oc2ccccc2)cc1)c1cc(C(=N)N)cs1] | (2S,4R)-N-((R)-1-(4- carbamimidoylthiophen- 2-yl)ethyl)-4- cyclohexyl-1-((4- phenoxybenzoyl)glycyl) pyrrolidine-2- carboxamide | *** | 1.90 | 602 |
| 242 | 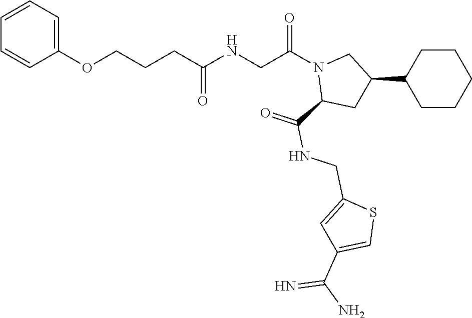 [SMILES: N=C(N)c1csc(CNC(=O)[C@@H]2C[C@H](C3CCCCC3)CN2C(=O)CNC(=O)CCCOc2ccccc2)c1] | (2S,4R)-N-((4- carbamimidoylthiophen- 2-yl)methyl)-4- cyclohexyl-1-((4- phenoxybutanoyl)glycyl) pyrrolidine-2- carboxamide | *** | 1.62 | 554 |
| 243 | 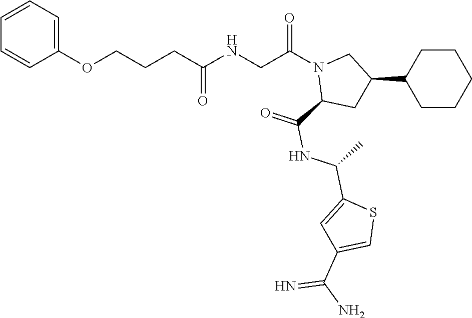 [SMILES: C[C@@H](NC(=O)[C@@H]1C[C@H](C2CCCCC2)CN1C(=O)CNC(=O)CCCOc1ccccc1)c1cc(C(=N)N)cs1] | (2S,4R)-N-((R)-1-(4- carbamimidoylthiophen- 2-yl)ethyl)-4- cyclohexyl-1-((4- phenoxybutanoyl)glycyl) pyrrolidine-2- carboxamide | *** | 1.69 | 568 |
| 244 | 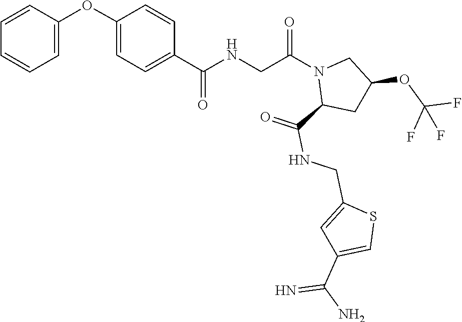 [SMILES: N=C(N)c1csc(CNC(=O)[C@@H]2C[C@H](OC(F)(F)F)CN2C(=O)CNC(=O)c2ccc(Oc3ccccc3)cc2)c1] | (2S,4S)-N-((4- carbamimidoylthiophen- 2-yl)methyl)-1-((4- phenoxybenzoyl)glycyl)- 4- (trifluoromethoxy) pyrrolidine- 2-carboxamide | *** | 1.51 | 590 |
| 245 | 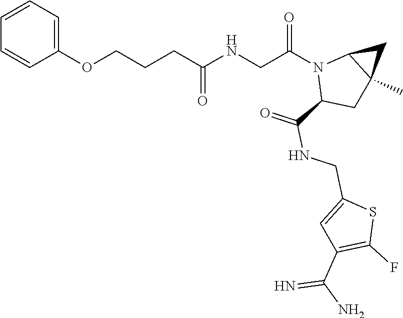 [SMILES: C[C@@]12C[C@@H]1N(C(=O)CNC(=O)CCCOc1ccccc1)[C@H](C(=O)NCc1cc(C(=N)N)c(F)s1)C2] | (1S,3S,5S)-N-((4- carbamimidoyl-5- fluorothiophen-2- yl)methyl)-5-methyl-2-((4- phenoxybutanoyl) glycyl)-2- azabicyclo[3.1.0]hexane- 3-carboxamide | *** | 1.18 | 516 |
| 246 | 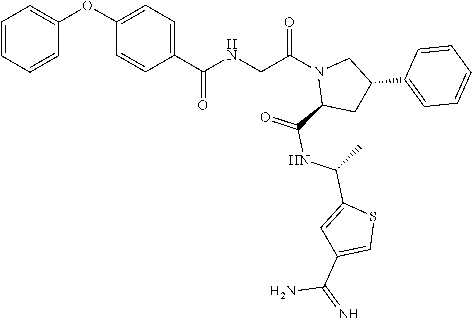 [SMILES: C[C@@H](NC(=O)[C@@H]1C[C@@H](c2ccccc2)CN1C(=O)CNC(=O)c1ccc(Oc2ccccc2)cc1)c1cc(C(=N)N)cs1] | (2S,4S)-N-((R)-1-(4- carbamimidoylthiophen- 2-yl)ethyl)-1-((4- phenoxybenzoyl)glycyl)- 4-phenylpyrrolidine-2- carboxamide | *** | 1.69 | 596 |
| 247 | 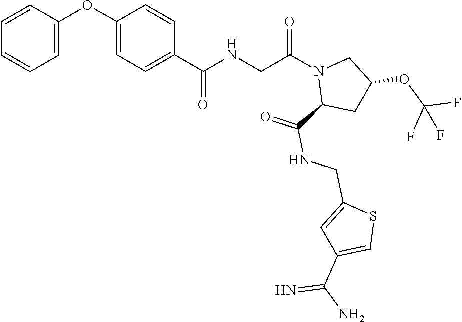 [SMILES: N=C(N)c1csc(CNC(=O)[C@@H]2C[C@@H](OC(F)(F)F)CN2C(=O)CNC(=O)c2ccc(Oc3ccccc3)cc2)c1] | (2S,4R)-N-((4- carbamimidoylthiophen- 2-yl)methyl)-1-((4- phenoxybenzoyl)glycyl)- 4-(trifluoromethoxy) pyrrolidine-2- carboxamide | *** | 1.44 | 590 |
| 248 | 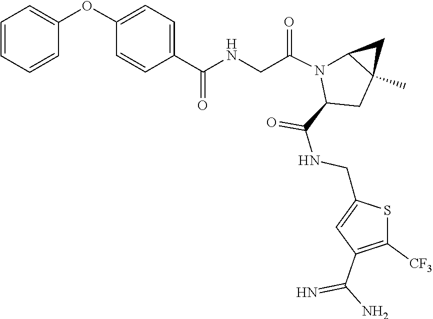 [SMILES: C[C@@]12C[C@@H]1N(C(=O)CNC(=O)c1ccc(Oc3ccccc3)cc1)[C@H](C(=O)NCc1cc(C(=N)N)c(C(F)(F)F)s1)C2] | (1S,3S,5S)-N-((4- carbamimidoyl-5- (trifluoromethyl)thiophen- 2-yl)methyl)-5- methyl-2-((4- phenoxybenzoyl)glycyl)-2- azabicyclo[3.1.0]hexane- 3-carboxamide | * | 1.59 | 600 |
| 249 | 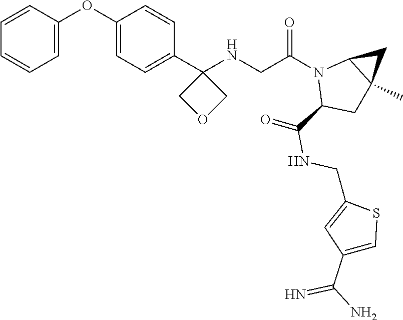 [SMILES: C[C@@]12C[C@@H]1N(C(=O)CNC1(c3ccc(Oc4ccccc4)cc3)COC1)[C@H](C(=O)NCc1cc(C(=N)N)cs1)C2] | (1S,3S,5S)-N-((4- carbamimidoylthiophen- 2-yl)methyl)-5-methyl- 2-((3-(4- phenoxyphenyl)oxetan- 3-yl)glycyl)-2- azabicyclo[3.1.0]hexane- 3-carboxamide | *** | 1.36 | 560 |
| 250 | 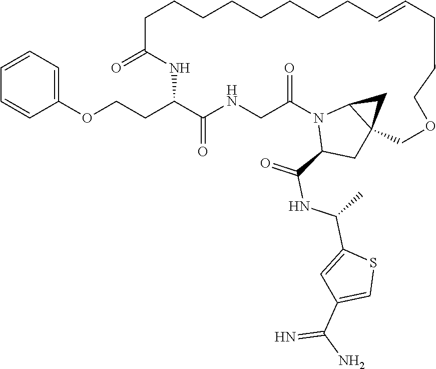 [SMILES: C[C@@H](NC(=O)[C@@H]1C[C@@]23COCCC/C=C/CCCCCCCCC(=O)N[C@@H](CCOc4ccccc4)C(=O)NCC(=O)N1[C@H]2C3)c1cc(C(=N)N)cs1] | (1R,19S,25S,28S,E)-N- ((R)-1-(4- carbamimidoylthiophen- 2-yl)ethyl)-17,20,23- trioxo-19-(2- phenoxyethyl)-3-oxa- 18,21,24- triazatricyclo[22.2.2.0 (1,25) ]octacos-7-ene-28- carboxamide | *** | 2.18 | 750 |
| 251 | 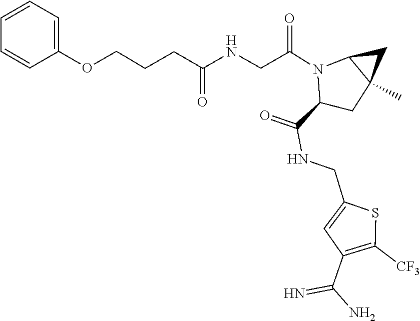 [SMILES: C[C@@]12C[C@@H]1N(C(=O)CNC(=O)CCCOc1ccccc1)[C@H](C(=O)NCc1cc(C(=N)N)c(C(F)(F)F)s1)C2] | (1S,3S,5S)-N-((4- carbamimidoyl-5- (trifluoromethyl)thiophen- 2-yl)methyl)-5- methyl-2-((4- phenoxybutanoyl)glycyl)- 2-azabicyclo[3.1.0]hexane- 3-carboxamide | * | 1.30 | 566 |
| 252 | 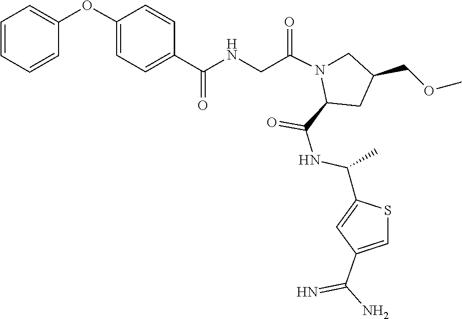 [SMILES: COC[C@H]1C[C@@H](C(=O)N[C@H](C)c2cc(C(=N)N)cs2)N(C(=O)CNC(=O)c2ccc(Oc3ccccc3)cc2)C1] | (2S,4S)-N-((R)-1-(4- carbamimidoylthiophen- 2-yl)ethyl)-4- (methoxymethyl)-1-((4- phenoxybenzoyl)glycyl) pyrrolidine-2- carboxamide | *** | 1.26 | 564 |
| 253 | 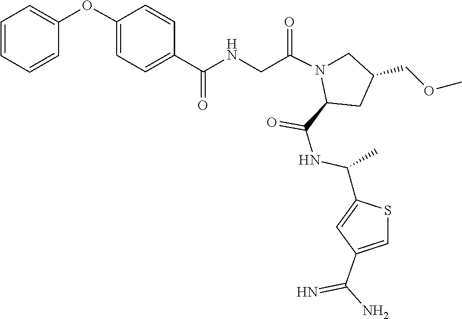 [SMILES: COC[C@@H]1C[C@@H](C(=O)N[C@H](C)c2cc(C(=N)N)cs2)N(C(=O)CNC(=O)c2ccc(Oc3ccccc3)cc2)C1] | (2S,4R)-N-((R)-1-(4- carbamimidoylthiophen- 2-yl)ethyl)-4- (methoxymethyl)-1-((4- phenoxybenzoyl)glycyl) pyrrolidine-2- carboxamide | *** | 1.28 | 564 |
| 254 | 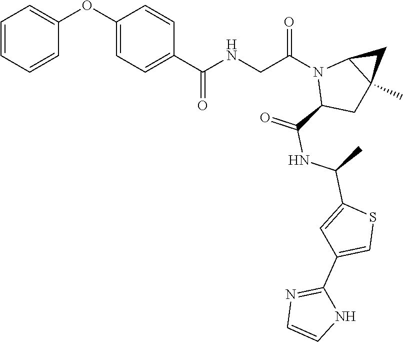 [SMILES: C[C@H](NC(=O)[C@@H]1C[C@]2(C)C[C@@H]2N1C(=O)CNC(=O)c1ccc(Oc2ccccc2)cc1)c1cc(-c2ncc[nH]2)cs1] | (1S,3S,5S)-N-((S)-1-(4- (1H-imidazol-2- yl)thiophen-2-yl)ethyl)- 5-methyl-2-((4- phenoxybenzoyl)glycyl)-2- azabicyclo[3.1.0]hexane- 3-carboxamide | * | 1.55 | 570 |
| 255 | 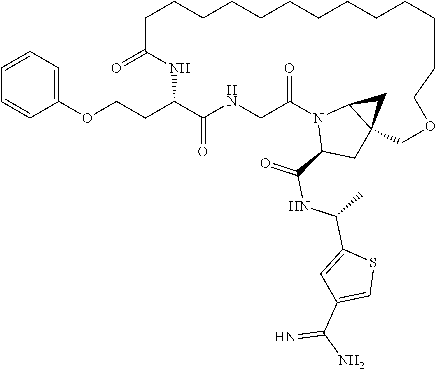 [SMILES: C[C@@H](NC(=O)[C@@H]1C[C@@]23COCCCCCCCCCCCCCC(=O)N[C@@H](CCOc4ccccc4)C(=O)NCC(=O)N1[C@H]2C3)c1cc(C(=N)N)cs1] | (1R, 19S,25S,28S)-N- ((R)-1-(4- carbamimidoylthiophen- 2-yl)ethyl)-17,20,23- trioxo-19-(2- phenoxyethyl)-3-oxa- 18,21,24- triazatricyclo[22.2.2.0 (1,25)]octacosane-28- carboxamide | *** | 2.10 | 752 |
| 256 | 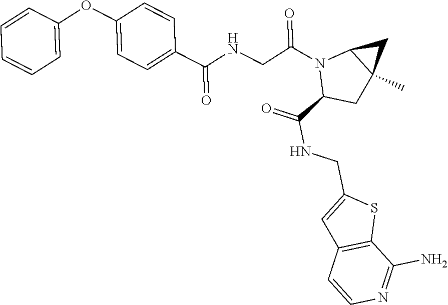 [SMILES: C[C@@]12C[C@@H]1N(C(=O)CNC(=O)c1ccc(Oc3ccccc3)cc1)[C@H](C(=O)NCc1cc3ccnc(N)c3s1)C2] | (1S,3S,5S)-N-((7- aminothieno[2,3- c]pyridin-2-yl)methyl)- 5-methyl-2-((4- phenoxybenzoyl) glycyl)-2- azabicyclo[3.1.0]hexane- 3-carboxamide | * | 1.35 | 556 |
| 257 | 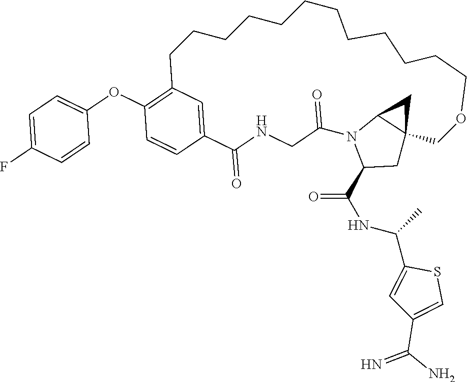 [SMILES: C[C@@H](NC(=O)[C@@H]1C[C@@]23COCCCCCCCCCCCCc4cc(ccc4Oc4ccc(F)cc4)C(=O)NCC(=O)N1[C@H]2C3)c1cc(C(=N)N)cs1] | (11R,13S,15S)- N-((R)-1-(4- carbamimidoylthiophen- 2-yl)ethyl)-64-(4- fluorophenoxy)-2,5- dioxo-19-oxa-14,4- diaza-1(4,1)- bicyclo[3.1.0]hexana- 6(1,3)- benzenacycloicosaphane- 13-carboxamide | * | 2.56 | 747 |
| 258 | 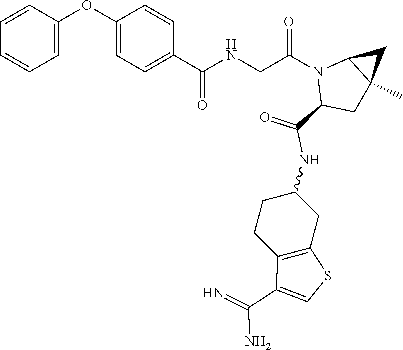 [SMILES: C[C@@]12C[C@@H]1N(C(=O)CNC(=O)c1ccc(Oc3ccccc3)cc1)[C@H](C(=O)NC1CCc3c(C(=N)N)csc3C1)C2] | (1S,3S,5S)-N-(3- carbamimidoyl-4,5,6,7- tetrahydrobenzo [b]thiophen- 6-yl)-5-methyl-2-((4- phenoxybenzoyl)glycyl)-2- azabicyclo[3.1.0]hexane- 3-carboxamide | * | 1.41 | 572 |
| 259 | 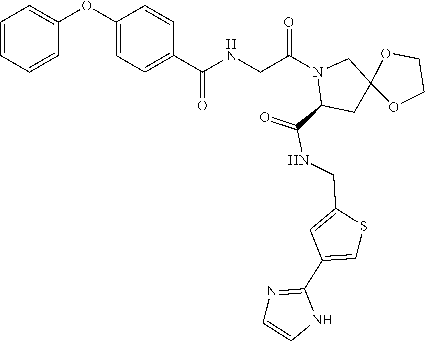 [SMILES: O=C(NCC(=O)N1CC2(C[C@H]1C(=O)NCc1cc(-c3ncc[nH]3)cs1)OCCO2)c1ccc(Oc2ccccc2)cc1] | (S)-N-((4-(1H-imidazol- 2-yl)thiophen-2- yl)methyl)-7-((4- phenoxybenzoyl)glycyl)- 1,4-dioxa-7- azaspiro[4.4]nonane-8- carboxamide | *** | 1.35 | 588 |
| 260 | 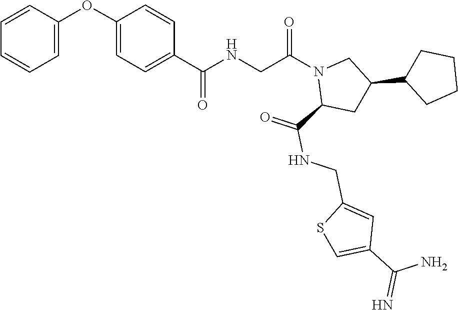 [SMILES: N=C(N)c1csc(CNC(=O)[C@@H]2C[C@H](C3CCCC3)CN2C(=O)CNC(=O)c2ccc(Oc3ccccc3)cc2)c1] | (2S,4R)-N-((4- carbamimidoylthiophen- 2-yl)methyl)-4- cyclopentyl-1-((4- phenoxybenzoyl)glycyl) pyrrolidine-2- carboxamide | *** | 1.69 | 575 |
| 261 | 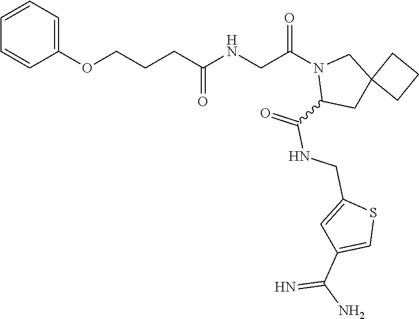 [SMILES: N=C(N)c1csc(CNC(=O)C2CC3(CCC3)CN2C(=O)CNC(=O)CCCOc2ccccc2)c1] | N-((4- carbamimidoylthiophen- 2-yl)methyl)-6-((4- phenoxybutanoyl) glycyl)-6- azaspiro[3.4]octane-7- carboxamide | *** | 1.09 | 512 |
| 262 | 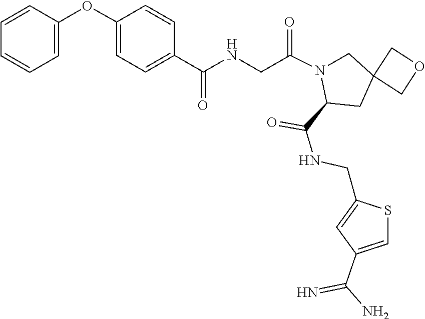 [SMILES: N=C(N)c1csc(CNC(=O)[C@@H]2CC3(COC3)CN2C(=O)CNC(=O)c2ccc(Oc3ccccc3)cc2)c1] | (S)-N-((4- carbamimidoylthiophen- 2-yl)methyl)-6-((4- phenoxybenzoyl)glycyl)- 2-oxa-6- azaspiro[3.4]octane-7- carboxamide | *** | 1.08 | 548 |
| 263 | 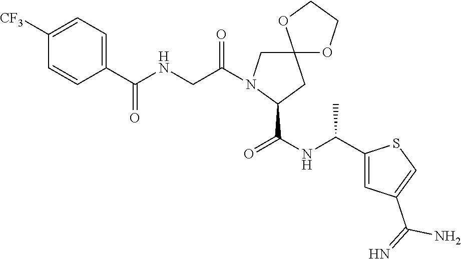 [SMILES: C[C@@H](NC(=O)[C@@H]1CC2(CN1C(=O)CNC(=O)c1ccc(C(F)(F)F)cc1)OCCO2)c1cc(C(=N)N)cs1] | (S)-N-((R)-1-(4- carbamimidoylthiophen- 2-yl)ethyl)-7-((4- (trifluoromethyl)benzoyl) glycyl)-1,4-dioxa-7- azaspiro[4.4]nonane-8- carboxamide | *** | 0.86 | 554 |
| 264 | 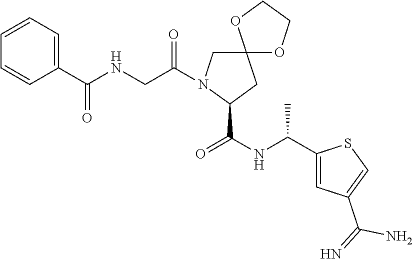 [SMILES: C[C@@H](NC(=O)[C@@H]1CC2(CN1C(=O)CNC(=O)c1ccccc1)OCCO2)c1cc(C(=N)N)cs1] | (S)-7-(benzoylglycyl)- N-((R)-1-(4- carbamimidoylthiophen- 2-yl)ethyl)-1,4-dioxa-7- azaspiro[4.4]nonane-8- carboxamide | *** | 0.36 | 486 |

### Example 3. Human C1s Enzyme Assay

Human complement C1s enzyme (purified from human serum, Complement Technology, Inc.) at 1.16 nM final concentration was incubated with test compound at various concentrations for 5 min at room temperature in 50 mM Tris, 1 M NaCl, pH 7.5. A synthetic substrate Z-L-Lys-SBzl and DTNB (Ellman's reagent) were added to final concentrations of 100 μM each. Absorbance at 405 nm (A405) was recorded at 30 second intervals for 30 min using a microplate spectrophotometer. IC₅₀ values were calculated by nonlinear regression of complement C1s reaction rates as a function of test compound concentration.

### Example 4. Hemolysis Assay

The hemolysis assay was previously described by Dodds, A. W. and Sim, R. B. (1997); Morgan, B. P. (2000). Prior to the assay, the optimum concentration of Normal Human Serum (NHS) needed to achieve 100% lysis of antibody sensitized sheep erythrocytes (EA) is determined by titration. EA are sheep erythrocytes with rabbit IgM anti-sheep erythrocyte antibodies bound to their surface. In the assay, NHS (Complement Technology) is diluted in GVB++ Buffer (0.1% gelatin, 5 mM Veronal, 145 mM NaCl, 0.025% NaN₃, pH 7.3, 0.15 mM calcium chloride and 0.5 mM magnesium chloride, Complement Technology) and incubated with test compound at various concentrations for 2 min at room temperature. EA (Complement Technology) freshly suspended in GVB++ are added to a final concentration of 1×10⁸ cells/mL and reactions are incubated for 60 min at 37° C. Positive control reactions (100% lysis) consist of GVB++ with NHS and EA but without test compound; negative control reactions (0% lysis) consist of GVB++ with EA only. Samples are centrifuged at 2000 g for 3 min and supernatants collected. Absorbance at 405 nm (A405) is recorded using a microplate spectrophotometer. IC₅₀ values are calculated by nonlinear regression from the percentage of hemolysis as a function of test compound concentration.

This specification has been described with reference to various specific embodiments. However, one of ordinary skill in the art appreciates that various modifications and changes can be made without departing from the scope of the invention as set forth in the claims. Accordingly, the specification is to be regarded in an illustrative rather than a restrictive sense, and all such modifications are intended to be included within the scope of the claims.

1. A compound selected from: 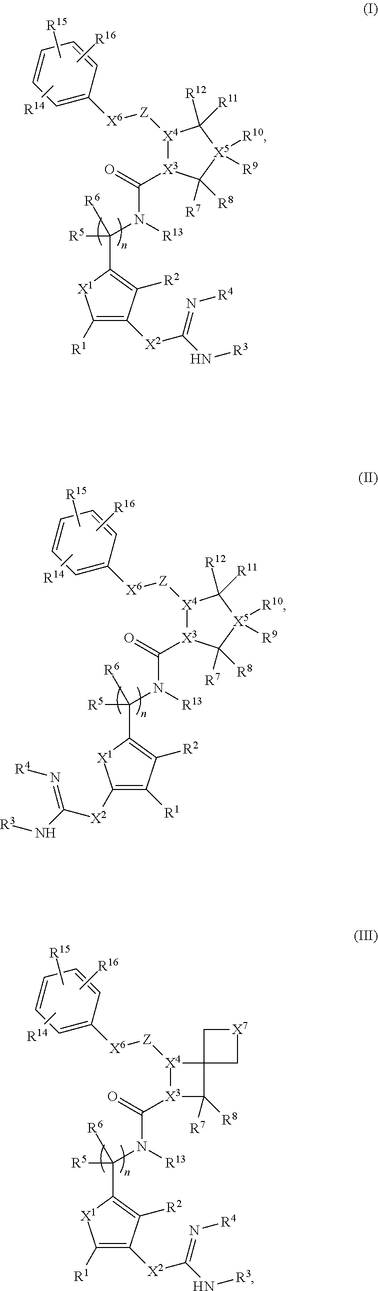 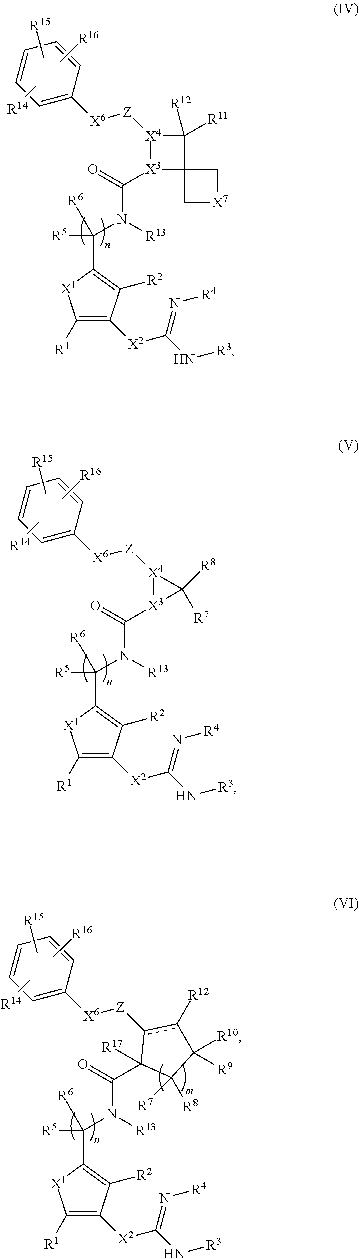 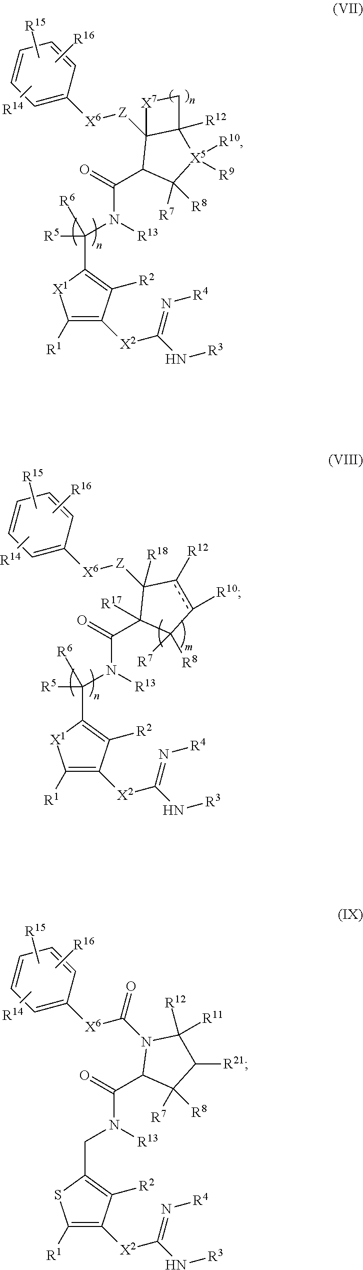 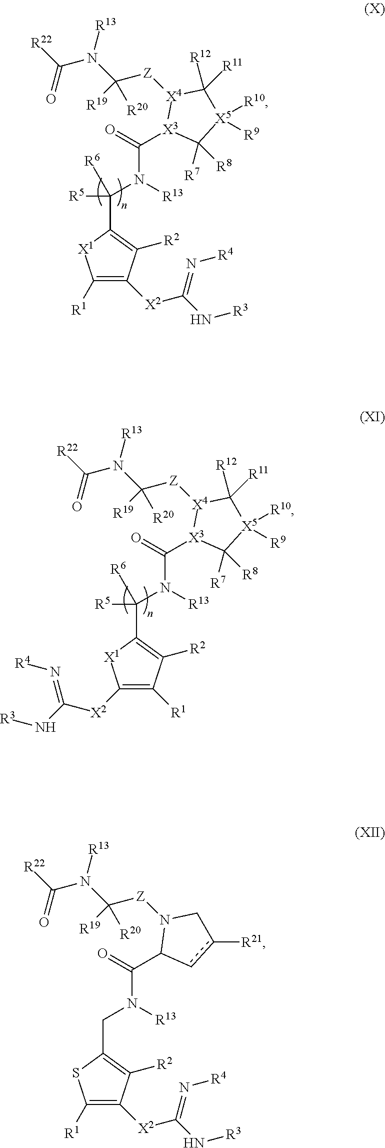 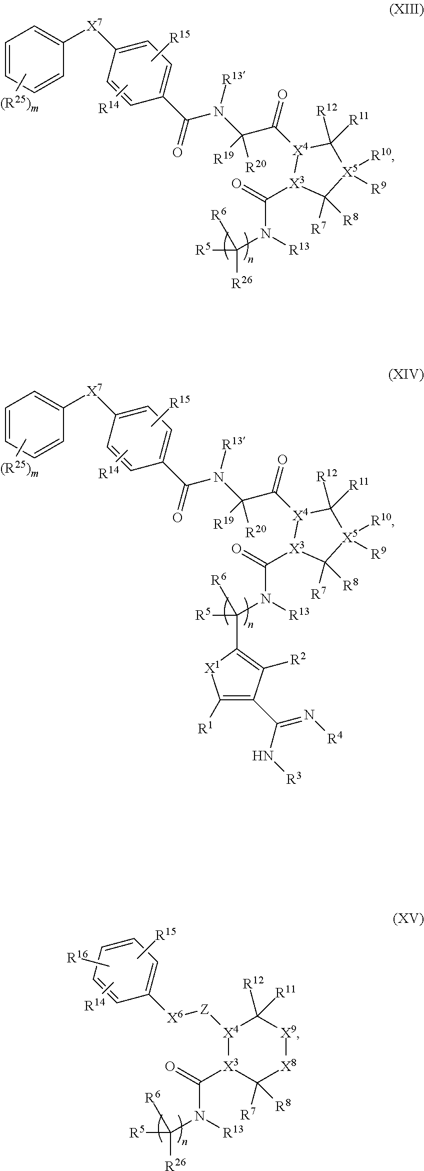 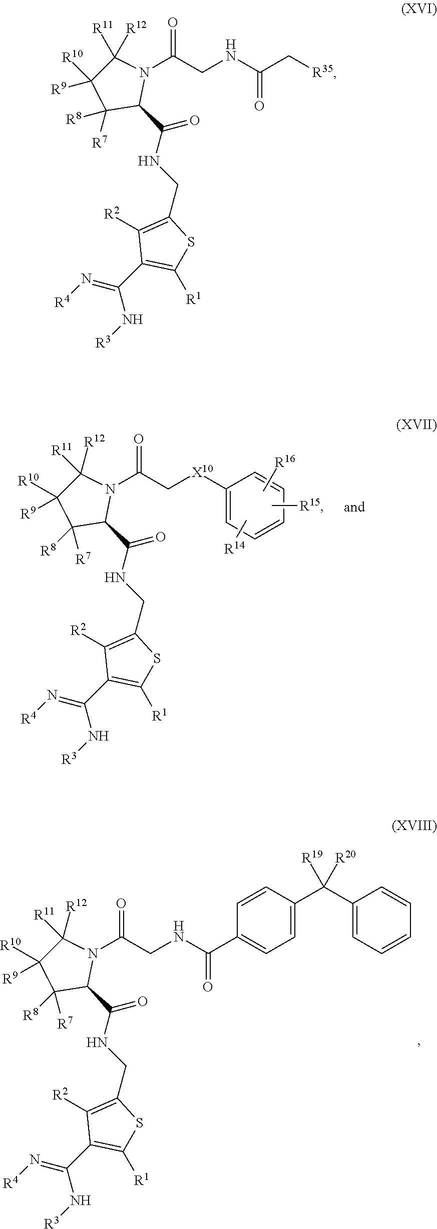 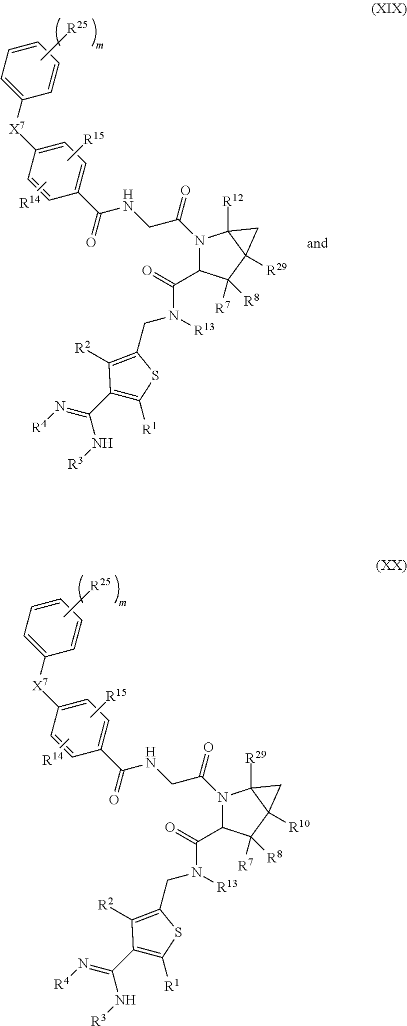
or a pharmaceutically acceptable salt, prodrug, or isolated isomer thereof;
wherein:
each n is independently 0, 1, 2, or 3;
each m is independently 0, 1, 2, or 3;
o is 0, 1, or 2; 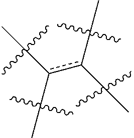
is either a single or a double bond;
Z is CH₂, C(CH₂), or C(O);
X¹ is selected from S, O, and N(R³⁰);
X² is selected from bond, N(R³⁰), and —O—N(R³⁰)—;
X³ is selected from N and C(R¹⁷);
X⁴ is selected from N and C(R¹⁸);
wherein only one of X³ and X⁴ can be N;
X⁵ is C, Si, or S;
X⁶ is selected from 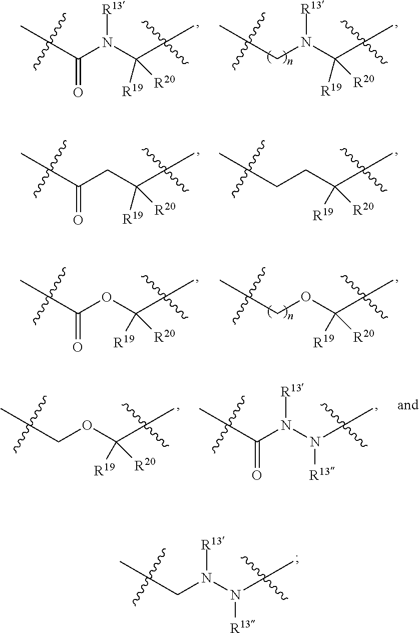
X⁷ is selected from O, S, N(R³⁰), and CR⁵R⁶;
each X⁸ and X⁹ is independently selected from O, S, NR³⁰, CR⁹R¹⁰, CR⁵R⁶. and CH₂; wherein X⁸ and X⁹ cannot both be the same group;
X¹⁰ is selected from 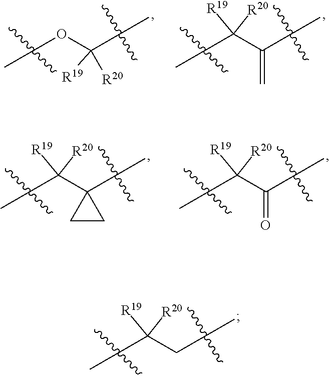
X¹¹ is selected from N and CR¹;
X¹² is selected from N and CR²;
R¹ and R² are independently selected from hydrogen, halogen, C₁-C₆alkyl, C₂-C₆alkenyl, C₂-C₆alkynyl, C₁-C₆haloalkyl, —OR³⁰, —SR³⁰, —N(R³⁰)₂, —C(O)R³¹, —S(O)R³¹, —S(O)₂R³¹, heterocycle, aryl, heteroaryl, cyano, and nitro; each of which R¹ and R² groups other than hydrogen, halogen, cyano, and nitro are optionally substituted with 1, 2, 3, or 4 substituents independently selected from C₁-C₆alkyl, C₂-C₆alkenyl, C₂-C₆alkynyl, halogen, C₁-C₆haloalkyl, —OR³⁰, —SR³⁰, —N(R³⁰)₂, —C(O)R³¹, —S(O)R³¹, —S(O)₂R³¹, heterocycle, aryl, heteroaryl, cyano, and nitro;
R³ and R⁴ are independently selected from hydrogen, nitro, —S(O)₂R³¹, CN, C(O)R³¹, —SR³⁰, and —OR³⁰;
or R³ and R⁴ are instead combined to form a dihydrooxadiazole optionally substituted with 1, 2, or 3 substituents independently selected from C₁-C₆alkyl, C₁-C₆haloalkyl, —OR³⁰, and oxo; an oxadiazole optionally substituted with 1, 2, or 3 substituents independently selected from C₁-C₆alkyl, C₁-C₆haloalkyl, and —OR³⁰; an imidazole optionally substituted with 1, 2, or 3 substituents independently selected from C₁-C₆alkyl, C₁-C₆haloalkyl, and —OR³⁰; or a dihydroimidazole optionally substituted 1, 2, or 3 substituents independently selected from C₁-C₆alkyl, C₁-C₆haloalkyl, —OR³⁰, and oxo;
R⁵ and R⁶ are each independently selected from hydrogen, halogen, C₁-C₆alkyl, C₁-C₆haloalkyl, C₁-C₆hydroxyalkyl, —OR³⁰, —N(R³⁰)₂, and COR³¹, wherein, when on carbons adjacent to each other, a R⁵ and a R⁶ group may optionally be replaced by a carbon-carbon double bond;
or, when n is 1, R⁵ and R⁶, together with the carbon to which they are attached, are replaced with —SO₂—;
or R⁵ and R⁶, together with the carbon atom to which they are attached, combine to form cyclopropyl;
R⁷, R⁸, R⁹, R¹⁰, R¹¹, and R¹² are independently selected from hydrogen, halogen, C₁-C₆alkyl, C₂-C₆alkenyl, C₂-C₆alkynyl, C₁-C₆haloalkyl, —OR³⁰, —SR³⁰, —N(R³⁰)₂, —C(O)R³¹, —S(O)R³¹, —S(O)₂R³¹, —S(O)(NR³¹)(R³¹), carbocycle, heterocycle, aryl, and heteroaryl, each of which R⁷, R⁸, R⁹, R¹⁰, R¹¹, and R¹² groups other than hydrogen and halogen are optionally substituted with 1, 2, 3, or 4 substituents independently selected from C₁-C₆alkyl, C₂-C₆alkenyl, C₂-C₆alkynyl, halogen, C₁-C₆haloalkyl, —OR³⁰, —SR³⁰, —N(R³⁰)₂, —C(O)R³¹, —S(O)R³¹, —S(O)₂R³¹, heterocycle, aryl, heteroaryl, cyano, nitro, and azido;
or R⁷ and R⁸ may be taken together with the carbon to which they are attached to form a 3- to 6-membered carbocyclic spiro ring or a 4- to 6-membered heterocyclic spiro ring containing 1 or 2 heteroatoms independently chosen from N, O, and S, wherein the carbocyclic spiro ring and heterocyclic spiro ring are optionally substituted with one or more halogen, C₁-C₆alkyl, C₁-C₆ haloalkyl, —OR³⁰, —SR³⁰, or —N(R³⁰)₂;
or R⁷ and R⁸ may be taken together with the carbon to which they are attached to form 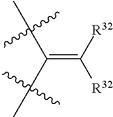
or carbonyl;
or R⁹ and R¹⁰ may be taken together with the atom to which they are attached to form a 3- to 6-membered carbocyclic spiro ring or a 4- to 6-membered heterocyclic spiro ring containing 1 or 2 heteroatoms independently chosen from N, O, and S, wherein the carbocyclic spiro ring and heterocyclic spiro ring are optionally substituted with one or more halogen, C₁-C₆alkyl, C₁-C₆ haloalkyl, —OR³⁰, —SR³⁰, or —N(R³⁰)₂;
or R⁹ and R¹⁰ may be taken together with the atom to which they are attached to form 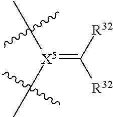
or carbonyl;
or R¹¹ and R¹² may be taken together with the carbon to which they are attached to form a 3- to 6-membered carbocyclic spiro ring or a 4- to 6-membered heterocyclic spiro ring containing 1 or 2 heteroatoms independently chosen from N, O, and S, wherein the carbocyclic spiro ring and heterocyclic spiro ring are optionally substituted with one or more halogen, C₁-C₆alkyl, C₁-C₆ haloalkyl, —OR³⁰, —SR³⁰, or —N(R³⁰)₂;
or R¹¹ and R¹² may be taken together with the carbon to which they are attached to form 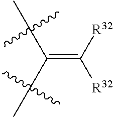
or carbonyl;
or R⁷ and R⁹ are taken together with the atoms to which they are attached to form a 3- to 8-membered carbocycle or a 4- to 8-membered heterocycle containing 1 or 2 heteroatoms independently chosen from N, O, and S;
or R⁹ and R¹¹ are taken together with the atoms to which they are attached to form a 3- to 8-membered carbocycle or a 4- to 8-membered heterocycle containing 1 or 2 heteroatoms independently chosen from N, O, and S;
or R⁷ and R¹¹ are taken together with the atoms to which they are attached to form a 1 or 2 carbon bridge;
or, when X⁵ is S, R⁹ and R¹⁰ are absent;
each R¹³ is independently selected from hydrogen, C₁-C₆alkyl, and OH;
or R¹³ and R²⁶, together with the atoms to which they are attached, form a heterocycle optionally substituted with R²⁷;
or R¹³, together with the nitrogen atom to which it is attached, is replaced with —O—;
each R^{13′} and R^{13″} is independently selected from hydrogen and C₁-C₆alkyl;
or R^{13′} and R¹⁴, together with the atoms to which they are attached, combine to form a 5- or 6-membered heterocycle containing one N;
R¹⁴, R¹⁵, and R¹⁶ are independently selected from hydrogen, halogen, SF₅, C₁-C₆alkyl, C₂-C₆alkenyl, C₂-C₆alkynyl, C₁-C₆haloalkyl, —C₁-C₆alkyl-aryl, —OR³⁰, —SR³⁰, —N(R³⁰)₂, —C(O)R³¹, —S(O)R³¹, —S(O)₂R³¹, carbocycle, heterocycle, aryl, heteroaryl, cyano, and nitro each of which R¹⁴, R¹⁵, and R¹⁶ groups other than hydrogen, halogen, cyano, and nitro are optionally substituted with 1, 2, 3, or 4 substituents independently selected from SF₅, C₁-C₆alkyl, C₂-C₆alkenyl, C₂-C₆alkynyl, halogen, C₁-C₆haloalkyl, —OR³⁰, —SR³⁰, —N(R³⁰)₂, —C(O)R³¹, —S(O)R³¹, —S(O)₂R³¹, carbocycle, heterocycle, aryl, heteroaryl, cyano, and nitro;
R¹⁷ and R¹⁸ are independently selected from hydrogen, halogen, C₁-C₆alkyl, C₁-C₆haloalkyl, —OR³⁰, and —N(R³⁰)₂;
or R¹⁷ and R¹⁸ are taken together with the carbons to which they are attached to form a double bond;
R¹⁹ and R²⁰ are independently selected from hydrogen, C₁-C₆alkyl, C₅-C₁₀ bicyclic carbocycle, C₄-C₆heterocycle, halogen, C₁-C₆haloalkyl, —OR³⁰, —N(R³⁰)₂, —(CH₂)ₙ—R³³, and 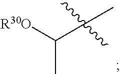
R²¹ is selected from C₁-C₆haloalkyl, —O—C₁-C₆haloalkyl, C₁-C₆alkyl, —O—C₁-C₆alkyl, —S(O)(NR³¹)R³¹, carbocycle, aryl, —O-aryl, heteroaryl, —O-carbocycle, or —O-heteroaryl, each of which R²¹ group is optionally substituted with 1, 2, 3, or 4 substituents independently selected from SF₅, C₁-C₆alkyl, C₂-C₆alkenyl, C₂-C₆alkynyl, halogen, C₁-C₆haloalkyl, —OR³⁰, —SR³⁰, —N(R³⁰)₂, —C(O)R³¹, —S(O)R³¹, —S(O)₂R³¹, heterocycle, aryl, heteroaryl, cyano, nitro, and azido;
R²² is selected from —C₁-C₆alkyl-R²³, —C₂-C₆alkenyl-R²³, —C₂-C₆alkynyl-R²³, -heteroaryl-R²³, -carbocycle-R²³, and bicyclic cycloalkyl-R²³, each of which R²² is optionally substituted with 1, 2, 3, or 4 substituents independently selected from C₁-C₆alkyl, C₂-C₆alkenyl, C₂-C₆alkynyl, halogen, C₁-C₆haloalkyl, —OR³⁰, —SR³⁰, —N(R³⁰)₂, —C(O)R³¹, —S(O)R³¹, —S(O)₂R³¹, heterocycle, aryl, heteroaryl, cyano, and nitro;
R²³ is selected from hydrogen, sugar, —OR³⁰, —SR³⁰, —N(R³⁰)₂, —C(O)R³¹, and —S(O)R³¹, —S(O)₂R³¹;
each R²⁵ is independently selected from hydrogen, SF₅, halogen, C₁-C₆alkyl, C₂-C₆alkenyl, C₂-C₆alkynyl, C₁-C₆haloalkyl, —OR³⁰, —SR³⁰, —N(R³⁰)₂, —C(O)R³¹, —S(O)R³¹, —S(O)₂R³¹, —S(O)(NR³¹)R³¹, —P(O)(OR³¹)R³¹, heterocycle, aryl, heteroaryl, cyano, and nitro; each of which R²⁵ groups other than hydrogen, halogen, cyano, and nitro are optionally substituted with 1, 2, 3, or 4 substituents independently selected from C₁-C₆alkyl, C₂-C₆alkenyl, C₂-C₆alkynyl, halogen, C₁-C₆haloalkyl, —OR³⁰, —SR³⁰, —N(R³⁰)₂, —C(O)R³¹, —S(O)R³¹, —S(O)₂R³¹, heterocycle, aryl, heteroaryl, cyano, and nitro;
R²⁵ is selected from 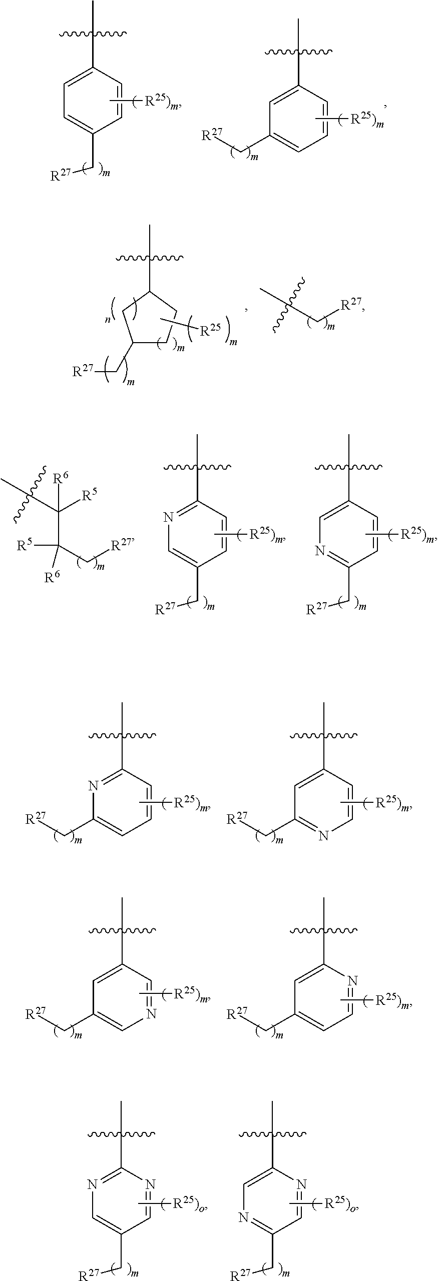 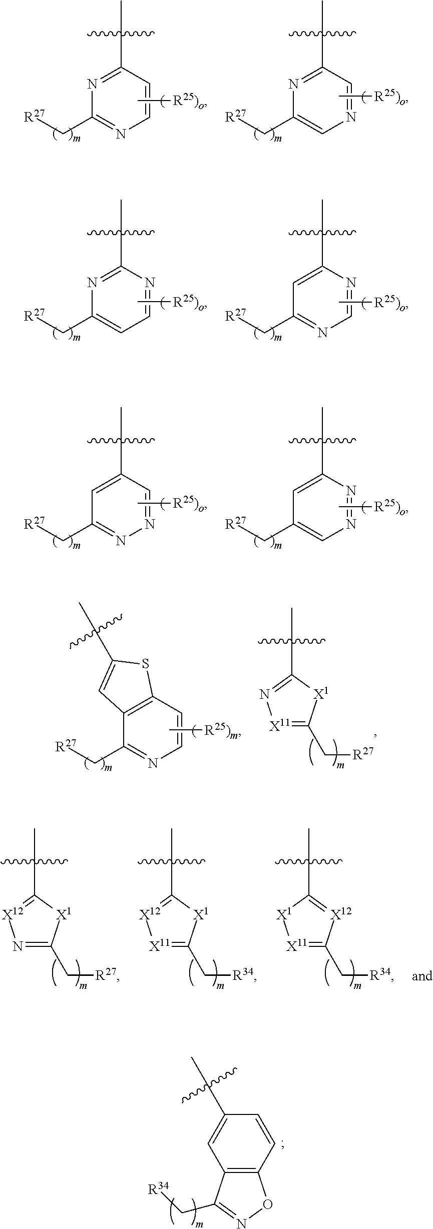
R²⁷ is selected from —OR³⁰, S-methylsulfonimidoyl, 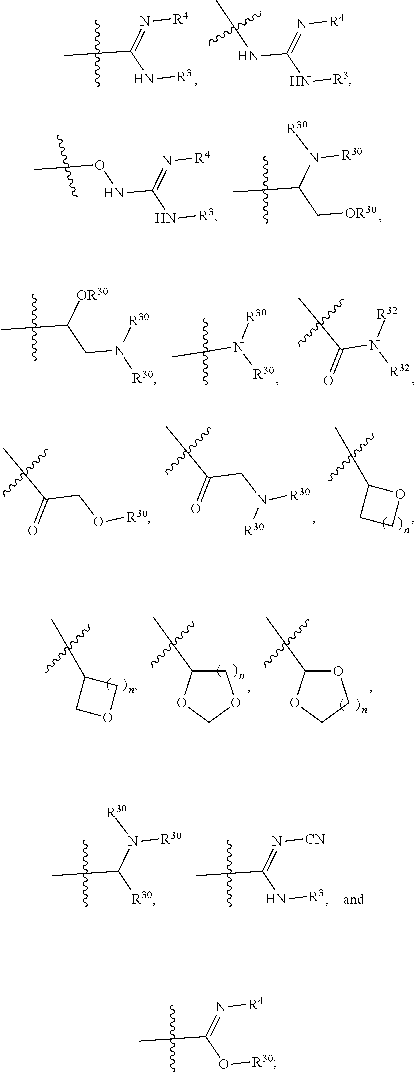
R²⁹ is selected from halogen, C₁-C₆alkyl, C₂-C₆alkenyl, C₂-C₆alkynyl, C₁-C₆haloalkyl, —OR³⁰, —SR³⁰, —N(R³⁰)₂, —C(O)R³¹, —S(O)R³¹, —S(O)₂R³¹, heterocycle, aryl, and heteroaryl, each of which R²⁹ groups other than hydrogen and halogen are optionally substituted with 1, 2, 3, or 4 substituents independently selected from C₁-C₆alkyl, C₂-C₆alkenyl, C₂-C₆alkynyl, halogen, C₁-C₆haloalkyl, —OR³⁰, —SR³⁰, —N(R³⁰)₂, —C(O)R³¹, —S(O)R³¹, —S(O)₂R³¹, heterocycle, aryl, heteroaryl, cyano, and nitro;
each R³⁰ is independently selected from hydrogen, C₁-C₆alkyl, C₁-C₆haloalkyl, carbocycle, aryl, heteroaryl, heterocycle, and C(O)R³¹, each R³⁰ other than C(O)R³¹ is optionally substituted with 1, 2, 3, or 4 substituents selected from C₁-C₆alkyl, halogen, SF₅, —C(O)R³¹, —N(R³⁰)₂, aryl, heteroaryl, —OR³², and —S(O)(NR³¹)R³¹, and carbocycle;
or R³⁰ and R⁴ in 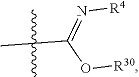
together with the N and O atoms to which each is attached and the carbon atom to which the N and O atoms are attached, combine to form oxadiazole optionally substituted with 1, 2, or 3 substituents independently selected from C₁-C₆alkyl, C₁-C₆haloalkyl, and —OR³⁰;
each R³¹ is independently selected from hydrogen, C₁-C₆alkyl, C₁-C₆haloalkyl, —OR³², —SR³², —N(R³²)₂, heterocycle, aryl, and heteroaryl;
each R³² is independently selected from hydrogen, halogen, C₁-C₆alkyl, and C₁-C₆haloalkyl;
each R³³ is independently selected from hydrogen, guanidine, heteroaryl, aryl, —C₆H₅—OR³⁰; —OR³⁰, —SR³⁰, —SeR³⁰, —N(R³⁰)₂, and —C(O)R³¹;
R³⁴ is selected from 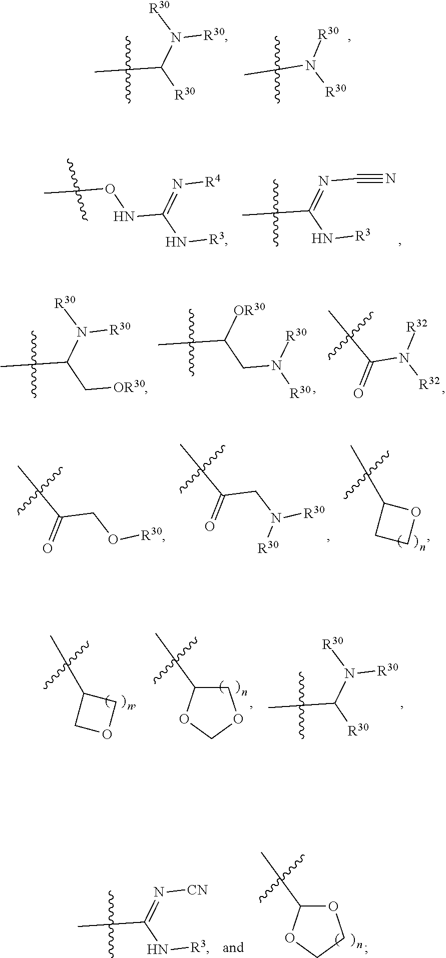
and R³⁵ is selected from C₃-C₁₀alkyl or C₃-C₁₀haloalkyl.

2. (canceled)

3. The compound of claim 1, wherein the compound is selected from: 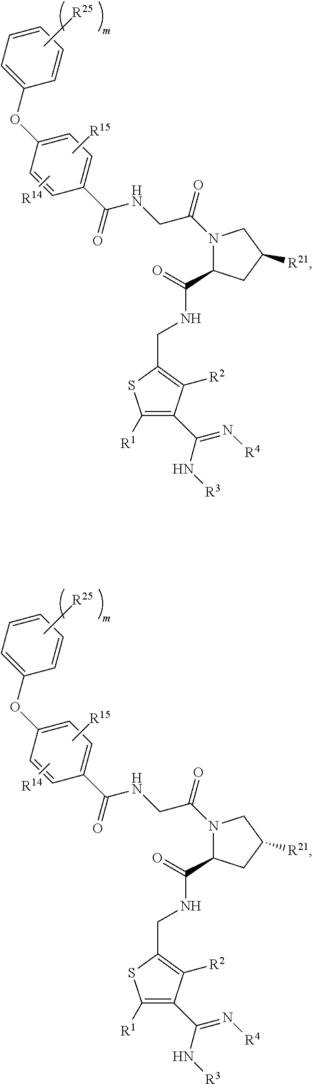 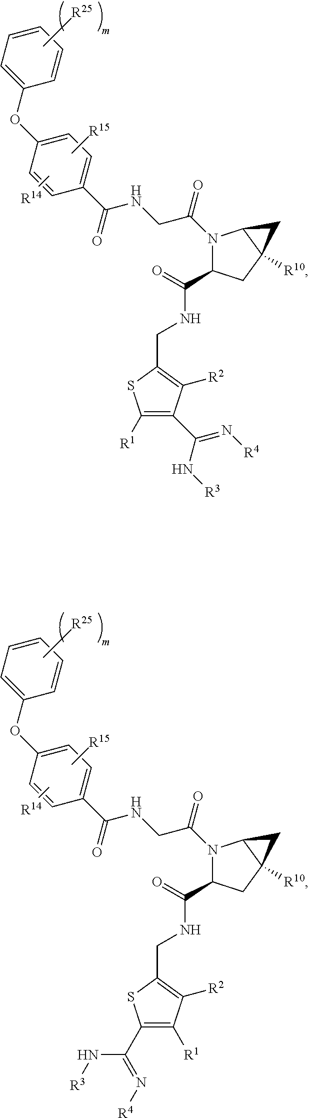 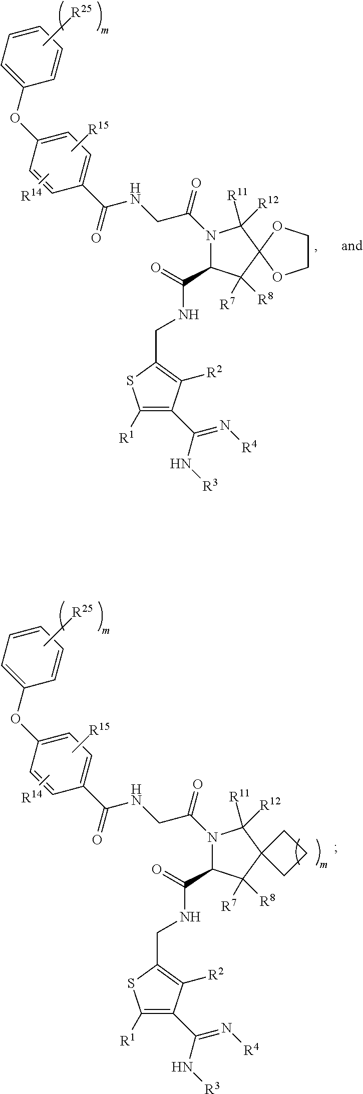
or a pharmaceutically acceptable salt, prodrug, or isolated isomer thereof.

4-8. (canceled)

9. The compound of claim 1, wherein R⁵ and R⁶, together with the carbon to which they are attached, is 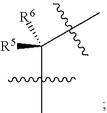
and R⁵ is methyl and R⁶ is H or R⁵ is H and R⁶ is H.

10-11. (canceled)

12. The compound of claim 1, wherein:
each m is independently 0 or 1; and/or
Z is C(O); and/or
X¹ is S; and/or
X² is a bond; and/or
X³ is C(R¹⁷); and/or
X⁴ is N; and/or
X⁵ is C.

13-20. (canceled)

21. The compound of claim 1, wherein X⁶ is

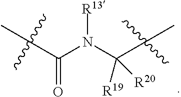

22. The compound of claim 1, wherein X⁷ is O.

23-26. (canceled)

27. The compound of claim 1, wherein R¹ and R² are both hydrogen; and/or R³ and R⁴ are both hydrogen.

28. (canceled)

29. The compound of claim 1, wherein R⁹ and R¹¹, together with the atoms to which they are attached, combine to form

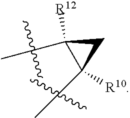

30. (canceled)

31. The compound of claim 1, wherein R¹⁰ is selected from halogen, C₁-C₆alkyl, C₂-C₆alkenyl, C₂-C₆alkynyl, C₁-C₆haloalkyl, —OR³⁰, —SR³⁰, —N(R³⁰)₂, —C(O)R³¹, —S(O)R³¹, and —S(O)₂R³¹, each R¹⁰ other than hydrogen and halogen are optionally substituted with 1, 2, 3, or 4 substituents independently selected from C₁-C₆alkyl, C₂-C₆alkenyl, C₂-C₆alkynyl, halogen, C₁-C₆haloalkyl, —OR³⁰, —SR³⁰, —N(R³⁰)₂, —C(O)R³¹, —S(O)R³¹, —S(O)₂R³¹, heterocycle, aryl, heteroaryl, cyano, nitro, and azido.

32-33. (canceled)

34. The compound of claim 1, wherein R⁹ and R¹⁰, together with the carbon atom to which they are attached, is 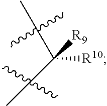
or R⁹ and R¹⁰ are combined to form a spirocycle.

35. The compound of claim 1, wherein R¹² is hydrogen; and/or R⁸ is hydrogen.

36. (canceled)

37. The compound of claim 1, wherein:
R¹³ is hydrogen, and/or
R¹⁹ is hydrogen; and/or
R²⁰ is hydrogen.

38-39. (canceled)

40. The compound of claim 1, wherein:
R¹⁴ is hydrogen, C₁-C₆alkyl, halogen, C₁-C₆haloalkyl, or —OR³⁰; and/or
R¹⁵ is hydrogen, C₁-C₆alkyl, halogen, C₁-C₆haloalkyl, or —OR³⁰; and/or
R¹⁶ is hydrogen, C₁-C₆alkyl, halogen, C₁-C₆haloalkyl, or —OR³⁰; and/or
R¹⁷ is hydrogen; and/or
R¹⁸ is hydrogen; and/or
R²⁵ is hydrogen, halogen, C₁-C₆alkyl, C₁-C₆haloalkyl, —OR³⁰, SF₅, S-methylsulfonimidoyl, or methylphosphinyl.

41-49. (canceled)

50. The compound of claim 1, wherein R²¹ is C₁-C₆haloalkyl; —O—C₁-C₆haloalkyl; phenyl optionally substituted with 1, 2, 3, or 4 substituents independently selected from SF₅, C₁-C₆alkyl, C₂-C₆alkenyl, C₂-C₆alkynyl, halogen, C₁-C₆haloalkyl, —OR³⁰, —SR³⁰, —N(R³⁰)₂, —C(O)R³¹, —S(O)R³¹, —S(O)₂R³¹, heterocycle, aryl, heteroaryl, cyano, and nitro; or is heteroaryl optionally substituted with 1, 2, 3, or 4 substituents independently selected from SF₅, C₁-C₆alkyl, C₂-C₆alkenyl, C₂-C₆alkynyl, halogen, C₁-C₆haloalkyl, —OR³⁰, —SR³⁰, —N(R³⁰)₂, —C(O)R³¹, —S(O)R³¹, —S(O)₂R³¹, heterocycle, aryl, heteroaryl, cyano, and nitro; and/or R²¹, together with the carbon to which it is attached, is

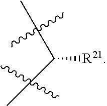

51. (canceled)

52. The compound of claim 1, wherein R²² is —C₁-C₆alkyl-R²³, bicyclic cycloalkyl-R²³, -heteroaryl-R²³, or -carbocycle-R²³, wherein R²² is optionally substituted with 1, 2, 3, or 4 substituents independently selected from C₁-C₆alkyl, C₂-C₆alkenyl, C₂-C₆alkynyl, halogen, C₁-C₆haloalkyl, —OR³⁰, —SR³⁰, —N(R³⁰)₂, —C(O)R³¹, —S(O)R³¹, —S(O)₂R³¹, heterocycle, aryl, heteroaryl, cyano, and nitro; and/or R²³ is —OR³⁰.

53-57. (canceled)

58. A compound selected from: 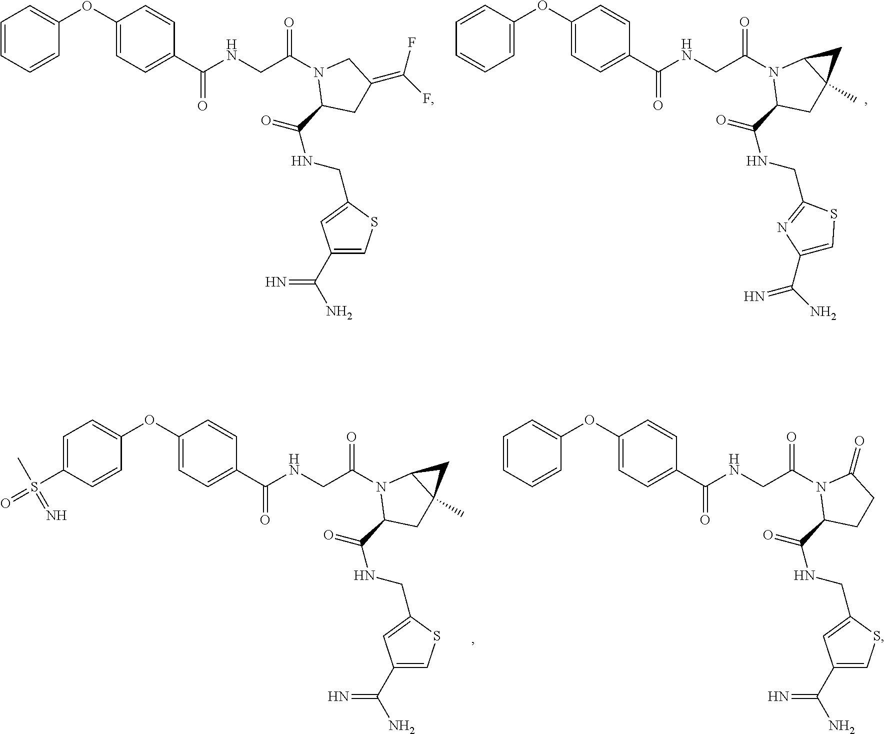 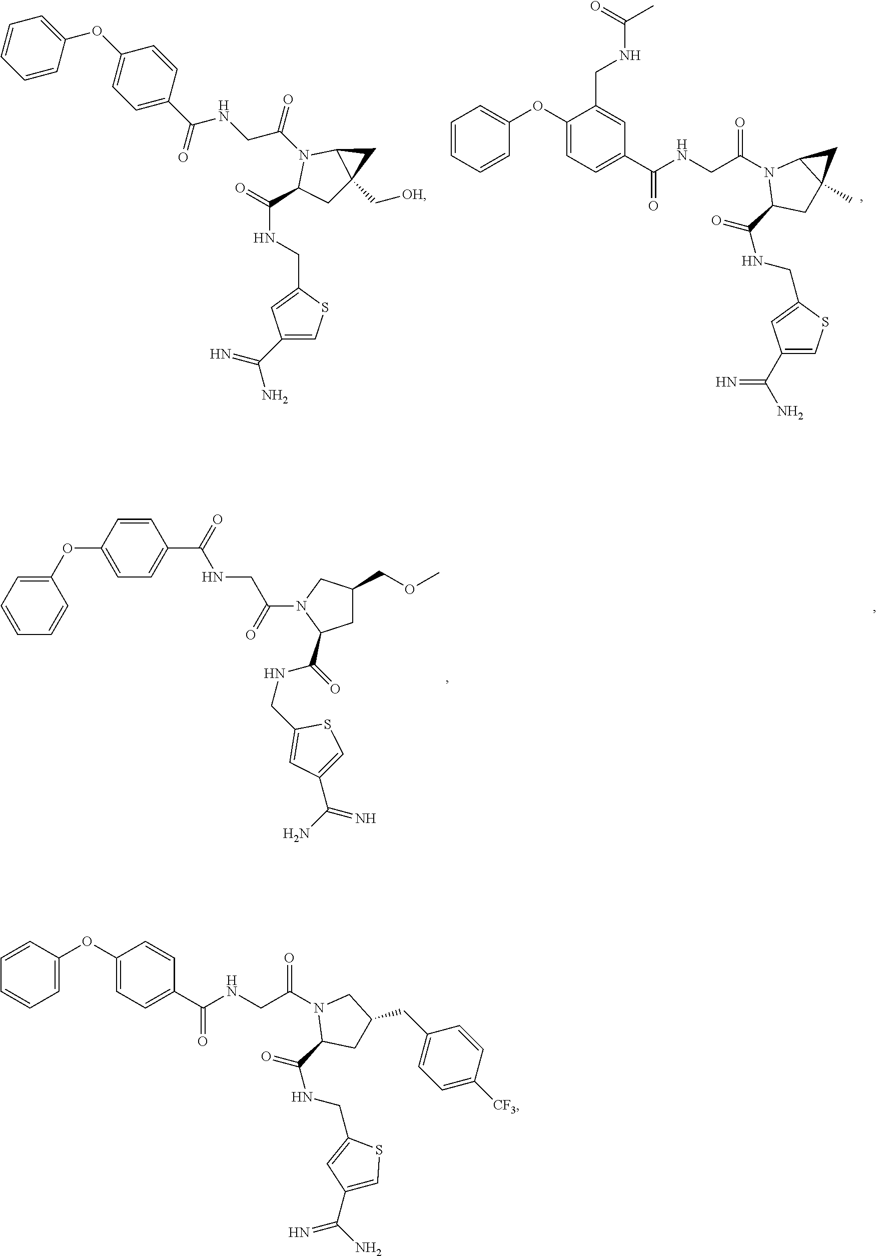 [SMILES: N=C(N)c1csc(CNC(=O)[C@@H]2C[C@]3(CO)C[C@@H]3N2C(=O)CNC(=O)c2ccc(Oc3ccccc3)cc2)c1 ; CC(=O)NCc1cc(C(=O)NCC(=O)N2[C@H]3C[C@@]3(C)C[C@H]2C(=O)NCc2cc(C(=N)N)cs2)ccc1Oc1ccccc1 ; COC[C@H]1C[C@@H](C(=O)NCc2cc(C(=N)N)cs2)N(C(=O)CNC(=O)c2ccc(Oc3ccccc3)cc2)C1 ; N=C(N)c1csc(CNC(=O)[C@@H]2C[C@@H](Cc3ccc(C(F)(F)F)cc3)CN2C(=O)CNC(=O)c2ccc(Oc3ccccc3)cc2)c1] 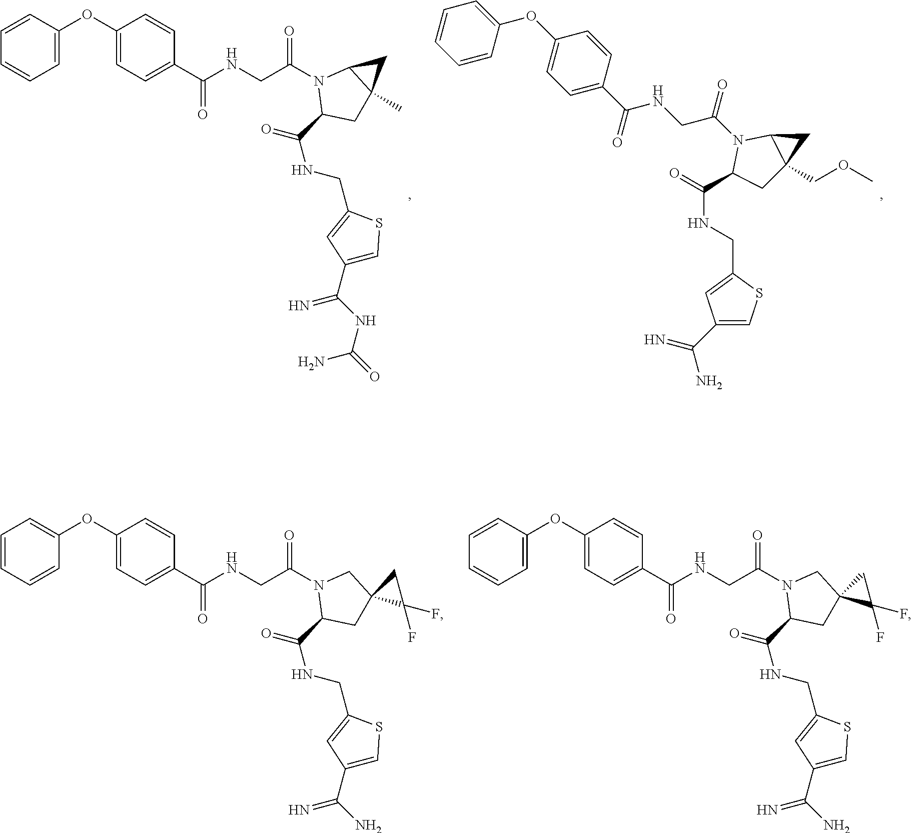 [SMILES: C[C@@]12C[C@@H]1N(C(=O)CNC(=O)c1ccc(Oc3ccccc3)cc1)[C@H](C(=O)NCc1cc(C(=N)NC(N)=O)cs1)C2 ; COC[C@@]12C[C@@H]1N(C(=O)CNC(=O)c1ccc(Oc3ccccc3)cc1)[C@H](C(=O)NCc1cc(C(=N)N)cs1)C2 ; N=C(N)c1csc(CNC(=O)[C@@H]2C[C@@]3(CN2C(=O)CNC(=O)c2ccc(Oc4ccccc4)cc2)CC3(F)F)c1 ; N=C(N)c1csc(CNC(=O)[C@@H]2C[C@]3(CN2C(=O)CNC(=O)c2ccc(Oc4ccccc4)cc2)CC3(F)F)c1] 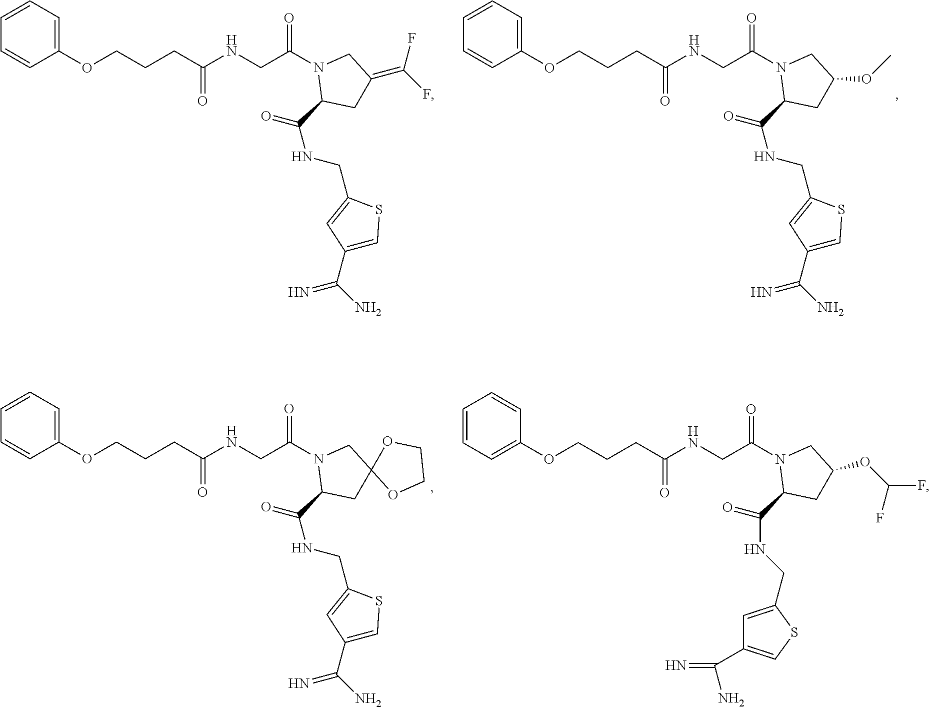 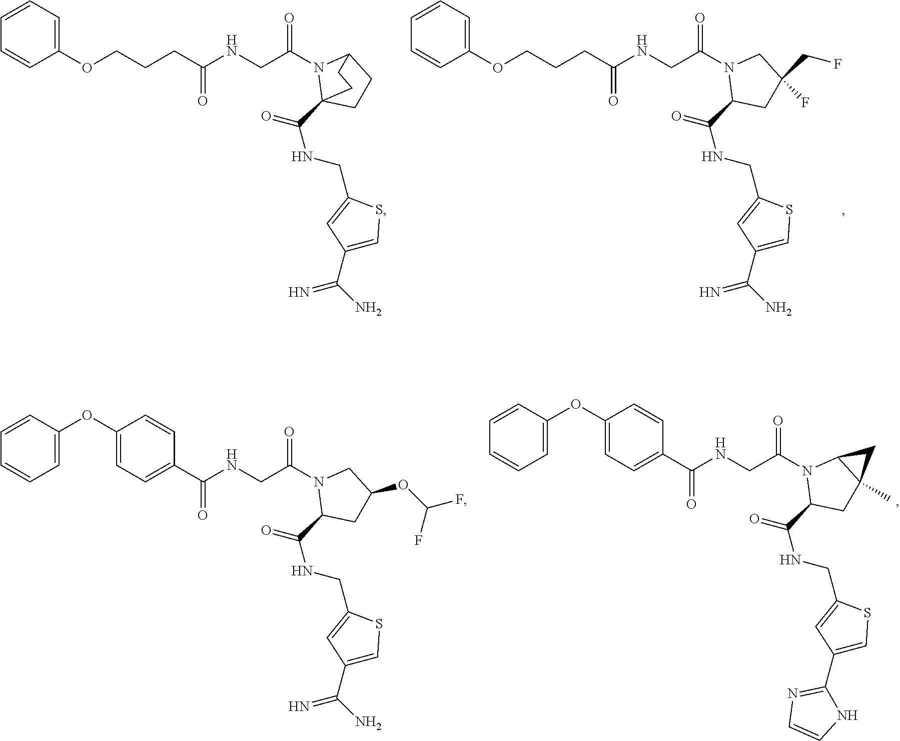 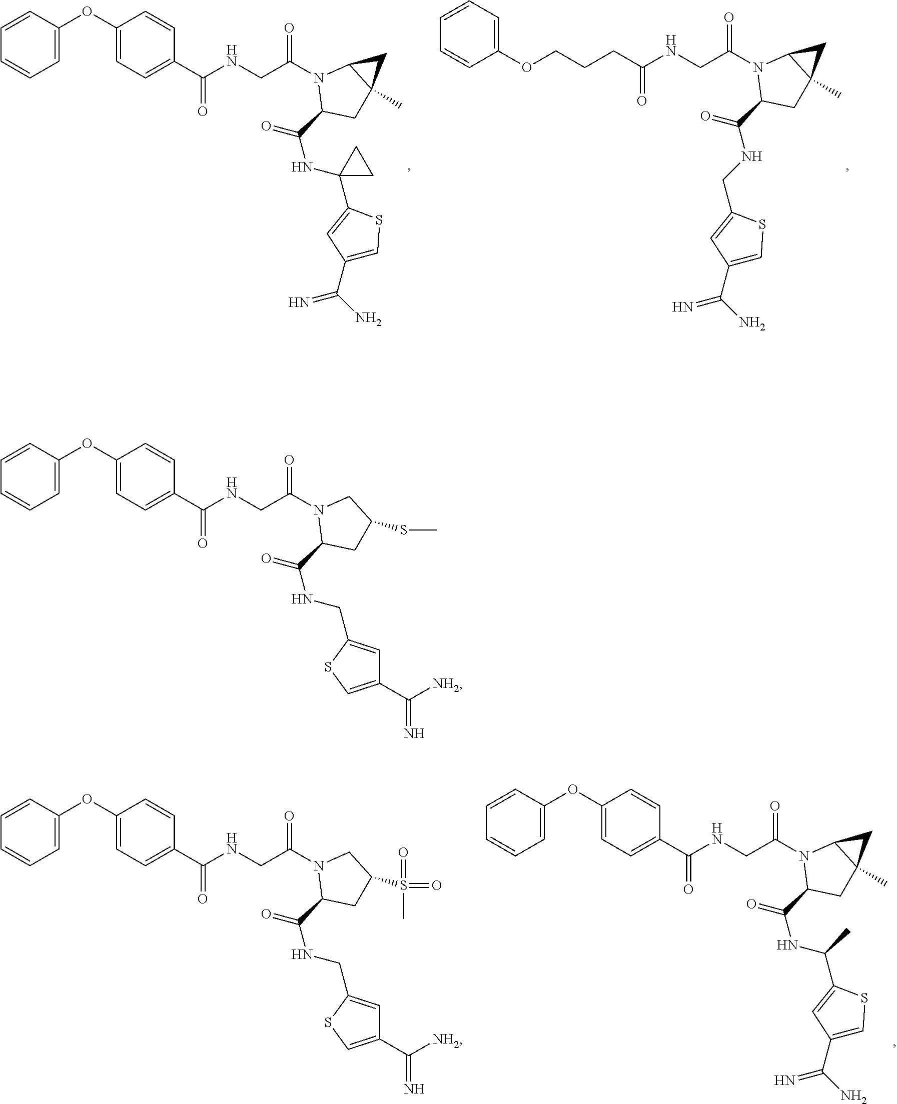 [SMILES: C[C@@]12C[C@@H]1N(C(=O)CNC(=O)c1ccc(Oc3ccccc3)cc1)[C@H](C(=O)NC1(c3cc(C(=N)N)cs3)CC1)C2 ; C[C@@]12C[C@@H]1N(C(=O)CNC(=O)CCCOc1ccccc1)[C@H](C(=O)NCc1cc(C(=N)N)cs1)C2 ; CS[C@@H]1C[C@@H](C(=O)NCc2cc(C(=N)N)cs2)N(C(=O)CNC(=O)c2ccc(Oc3ccccc3)cc2)C1 ; CS(=O)(=O)[C@@H]1C[C@@H](C(=O)NCc2cc(C(=N)N)cs2)N(C(=O)CNC(=O)c2ccc(Oc3ccccc3)cc2)C1 ; C[C@H](NC(=O)[C@@H]1C[C@]2(C)C[C@@H]2N1C(=O)CNC(=O)c1ccc(Oc2ccccc2)cc1)c1cc(C(=N)N)cs1] 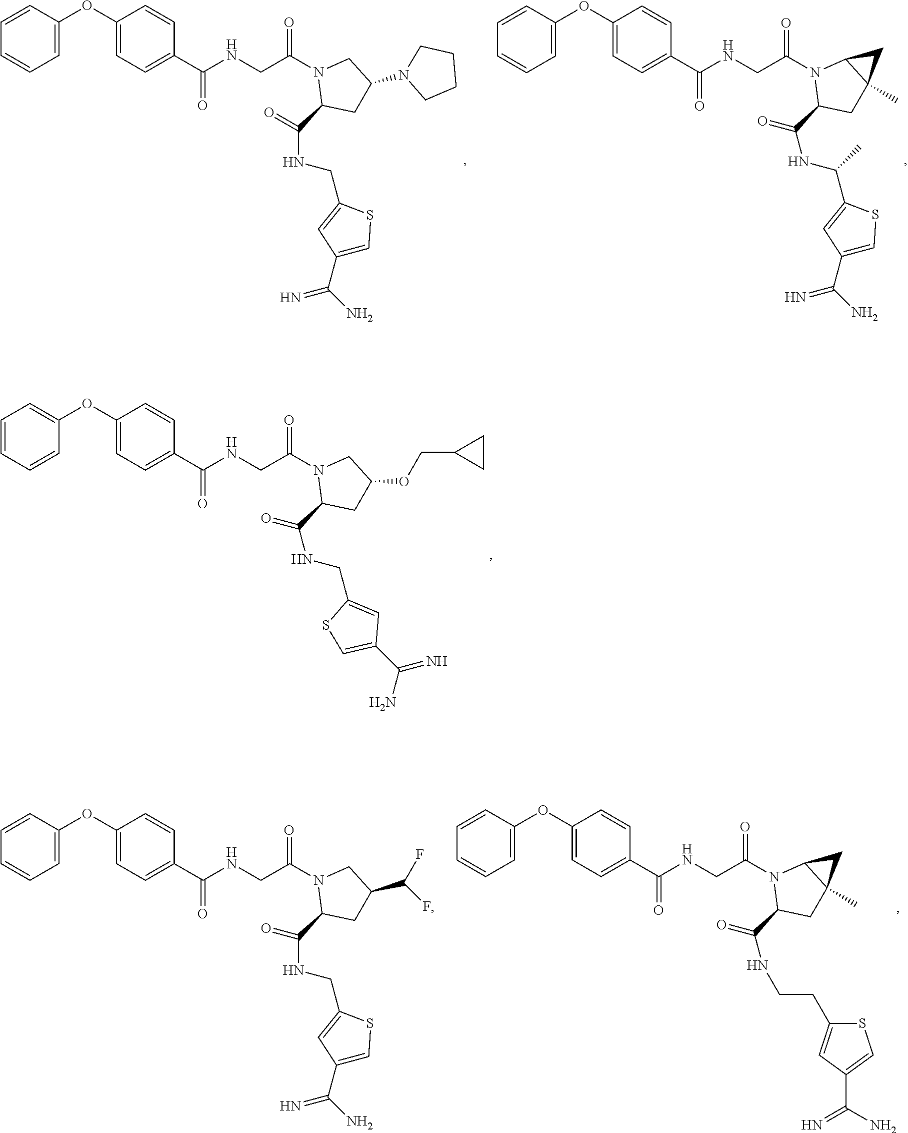 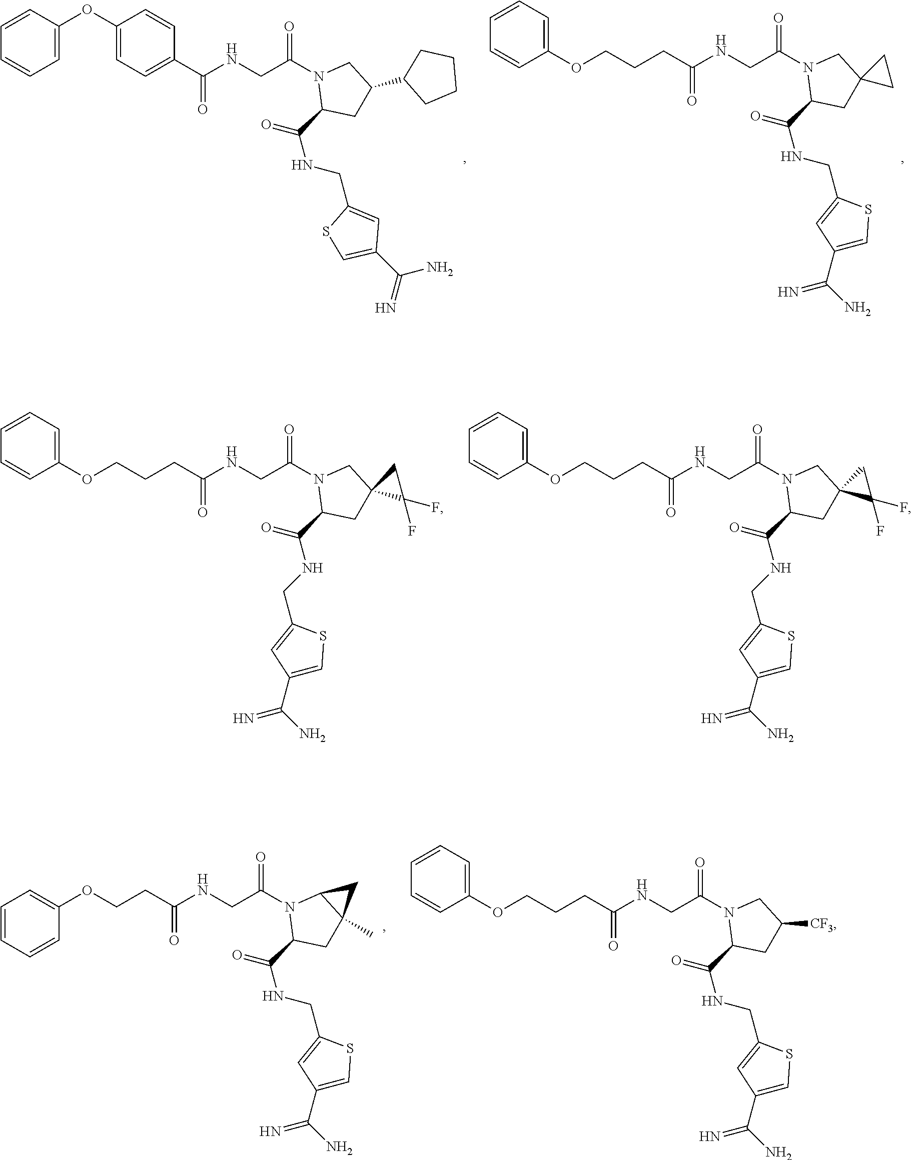 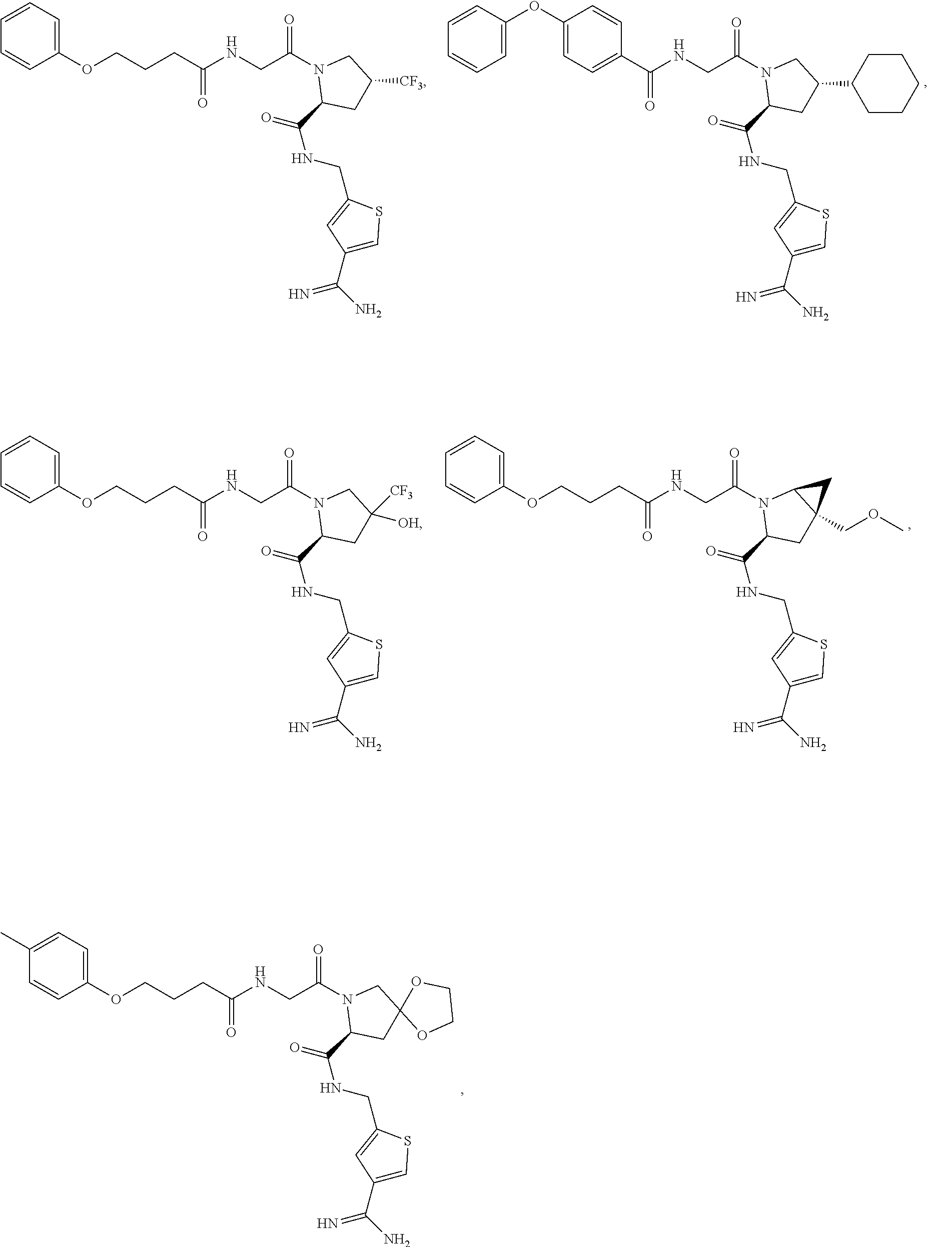 [SMILES: N=C(N)c1csc(CNC(=O)[C@@H]2C[C@@H](C(F)(F)F)CN2C(=O)CNC(=O)CCCOc2ccccc2)c1 ; N=C(N)c1csc(CNC(=O)[C@@H]2C[C@@H](C3CCCCC3)CN2C(=O)CNC(=O)c2ccc(Oc3ccccc3)cc2)c1 ; N=C(N)c1csc(CNC(=O)[C@@H]2CC(O)(C(F)(F)F)CN2C(=O)CNC(=O)CCCOc2ccccc2)c1 ; COC[C@@]12C[C@@H]1N(C(=O)CNC(=O)CCCOc1ccccc1)[C@H](C(=O)NCc1cc(C(=N)N)cs1)C2 ; Cc1ccc(OCCCC(=O)NCC(=O)N2CC3(C[C@H]2C(=O)NCc2cc(C(=N)N)cs2)OCCO3)cc1] 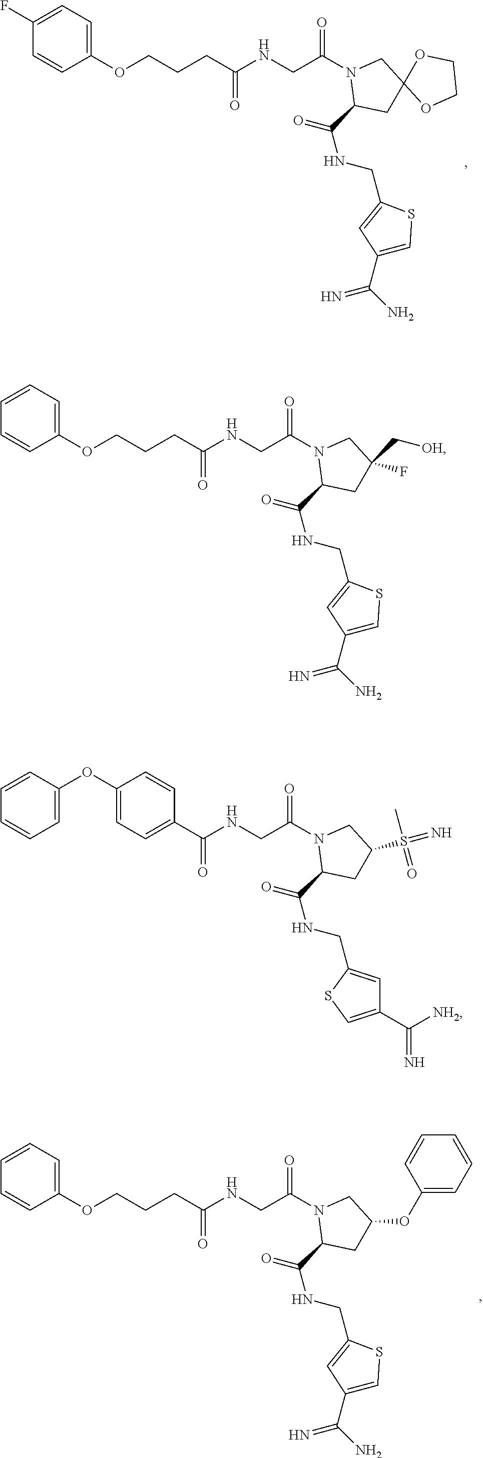 [SMILES: N=C(N)c1csc(CNC(=O)[C@@H]2CC3(CN2C(=O)CNC(=O)CCCOc2ccc(F)cc2)OCCO3)c1 ; N=C(N)c1csc(CNC(=O)[C@@H]2C[C@](F)(CO)CN2C(=O)CNC(=O)CCCOc2ccccc2)c1 ; CS(=N)(=O)[C@@H]1C[C@@H](C(=O)NCc2cc(C(=N)N)cs2)N(C(=O)CNC(=O)c2ccc(Oc3ccccc3)cc2)C1 ; N=C(N)c1csc(CNC(=O)[C@@H]2C[C@@H](Oc3ccccc3)CN2C(=O)CNC(=O)CCCOc2ccccc2)c1] 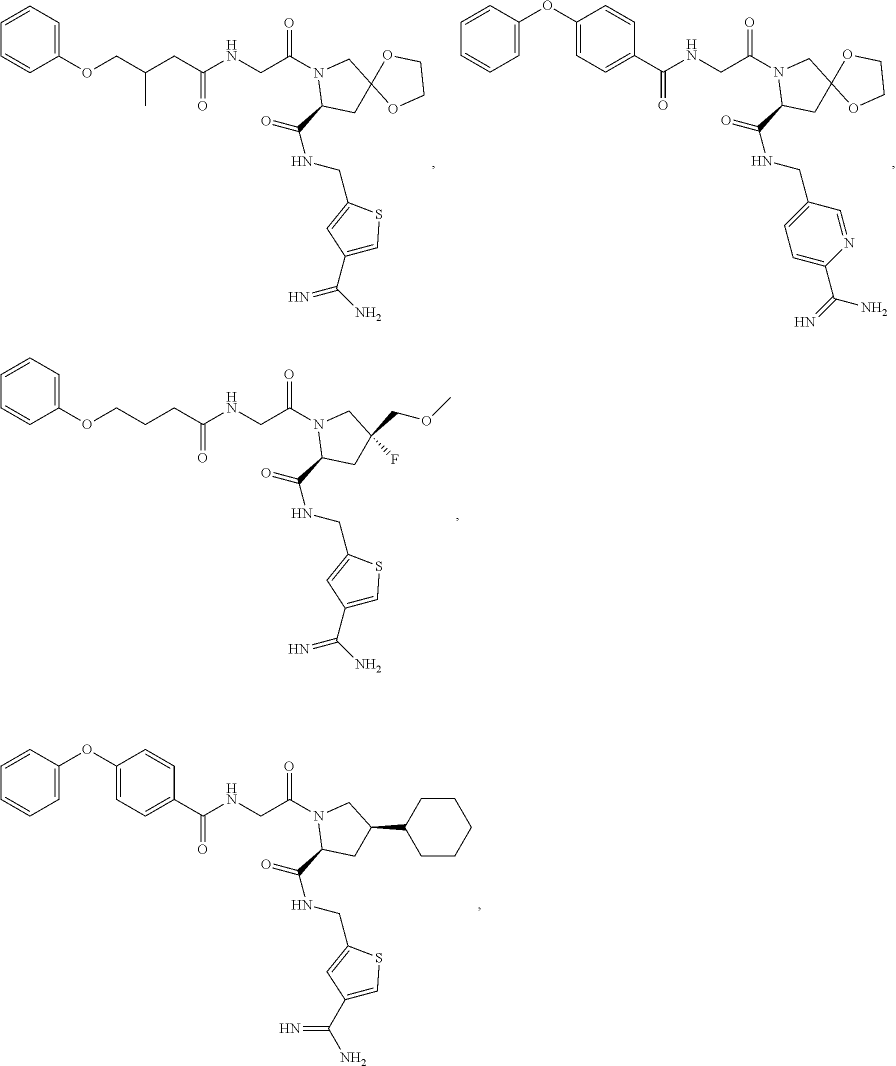 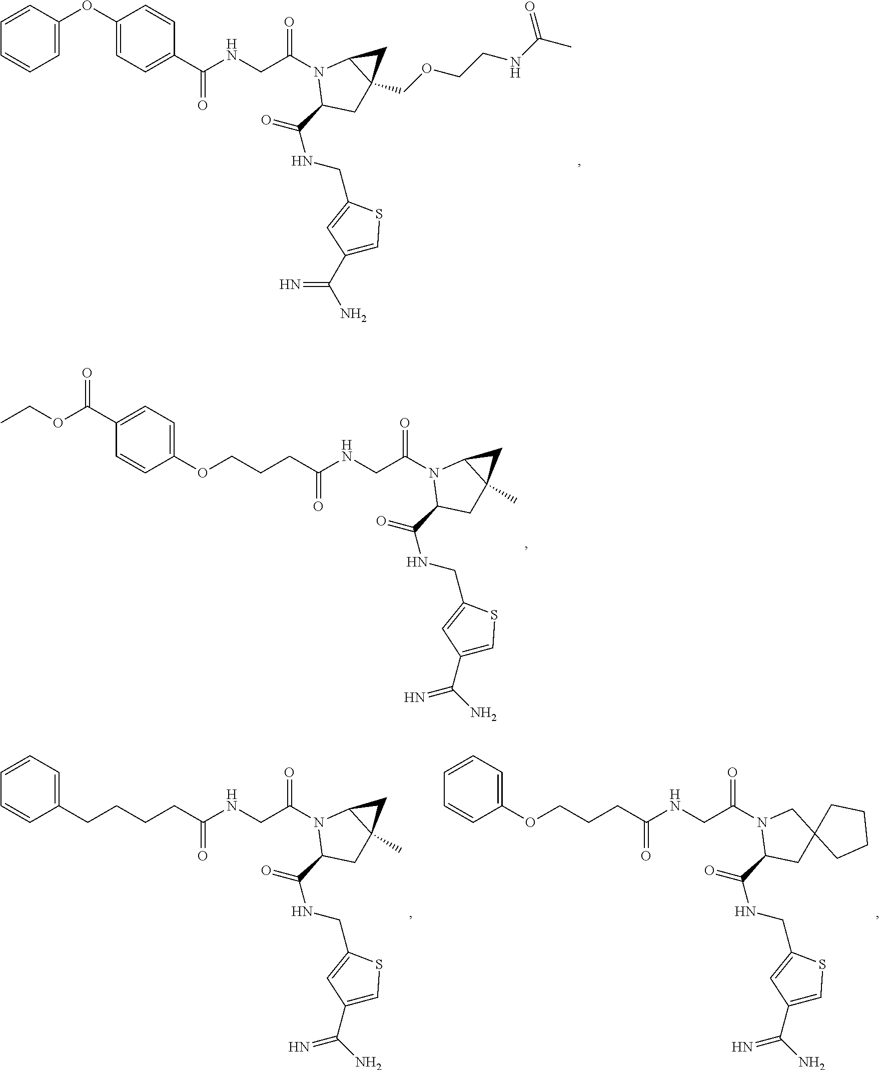 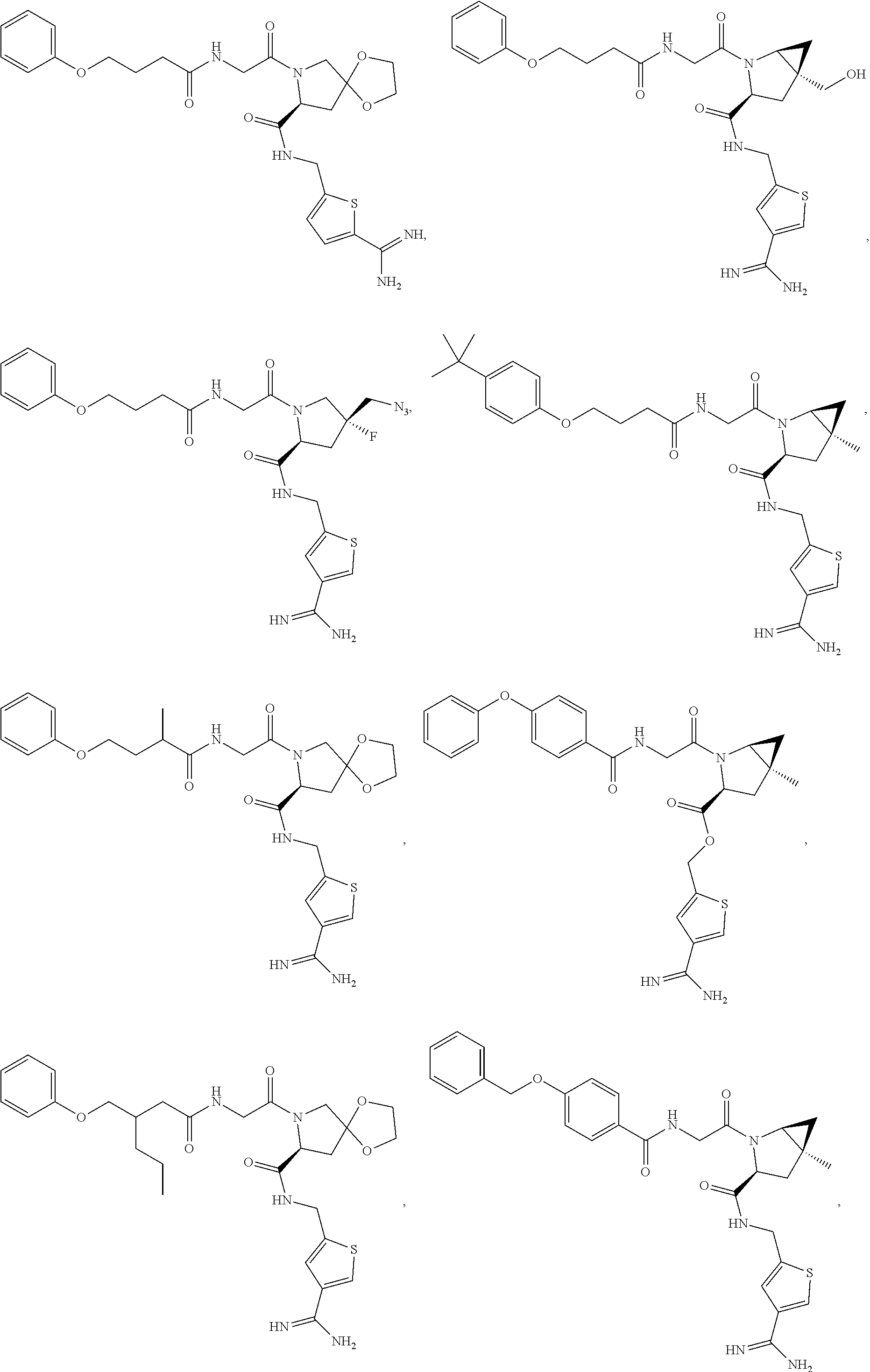 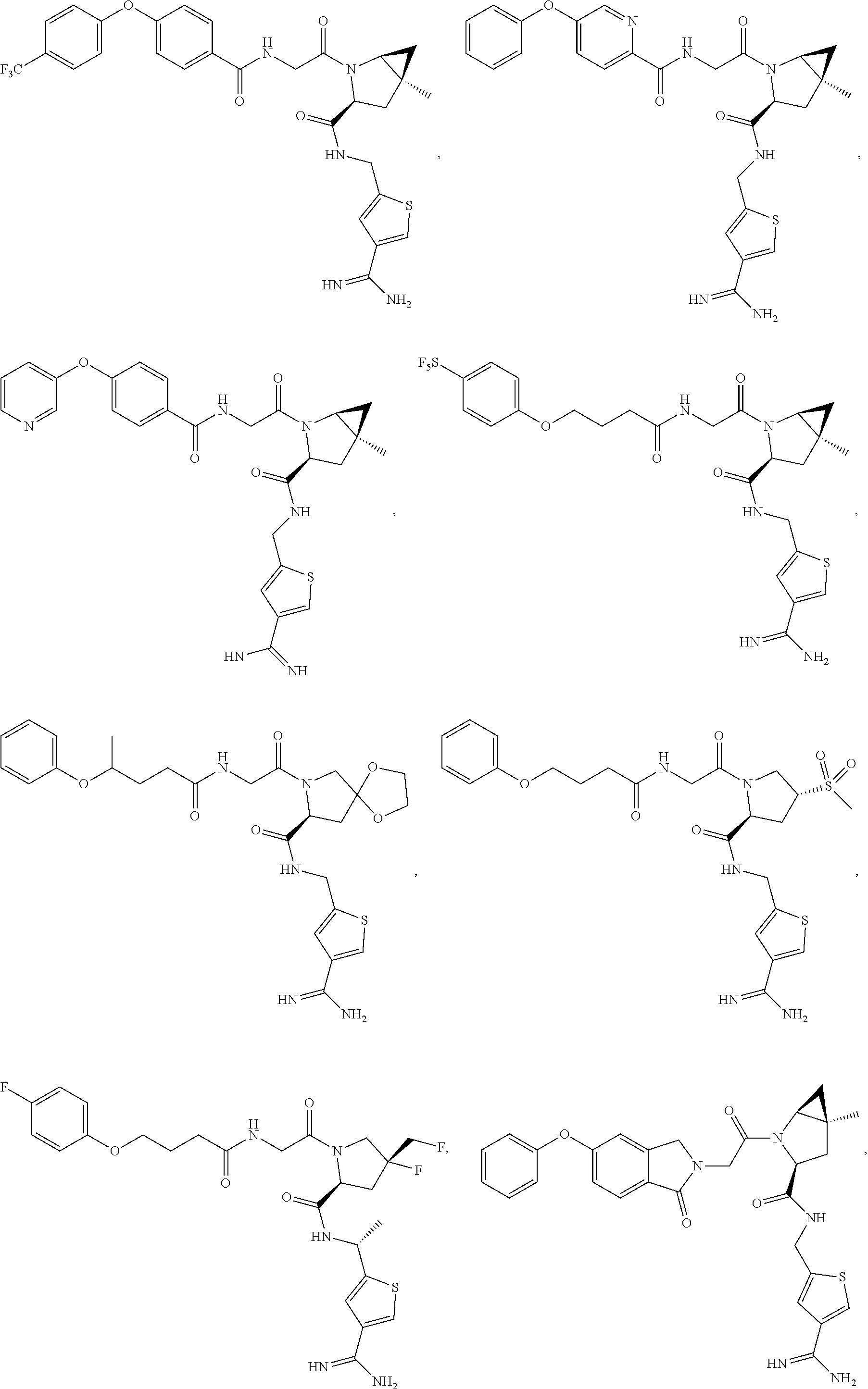 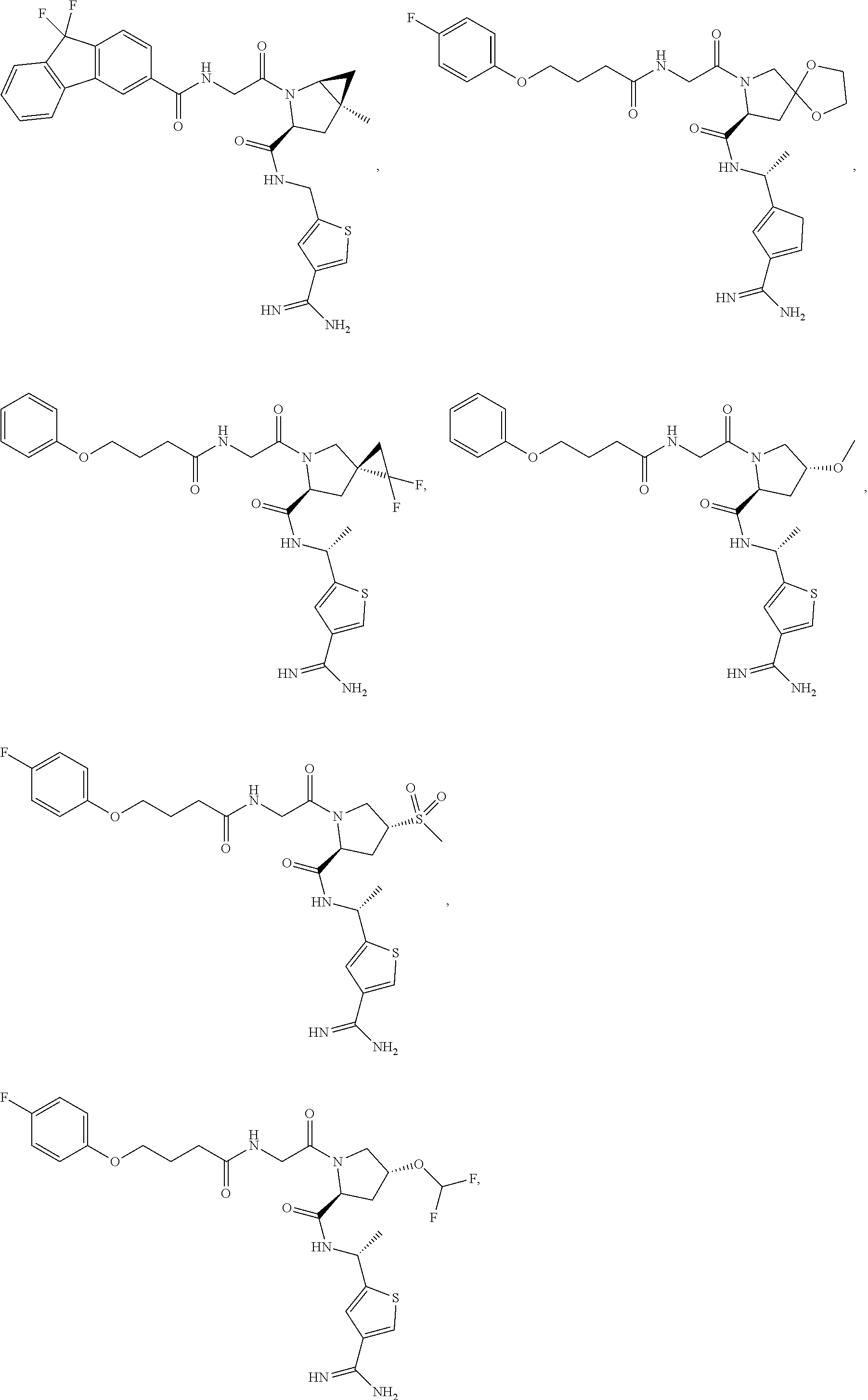 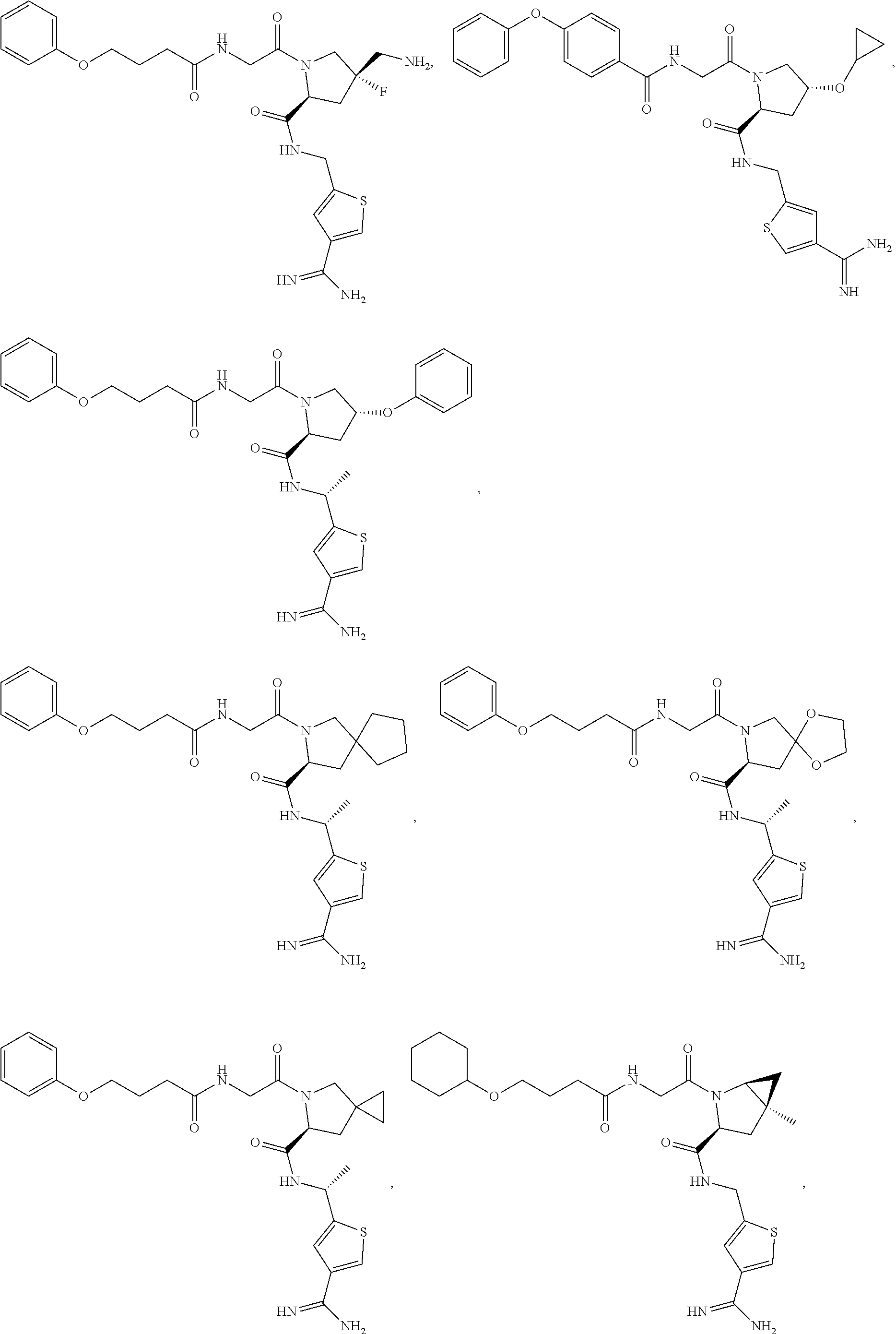 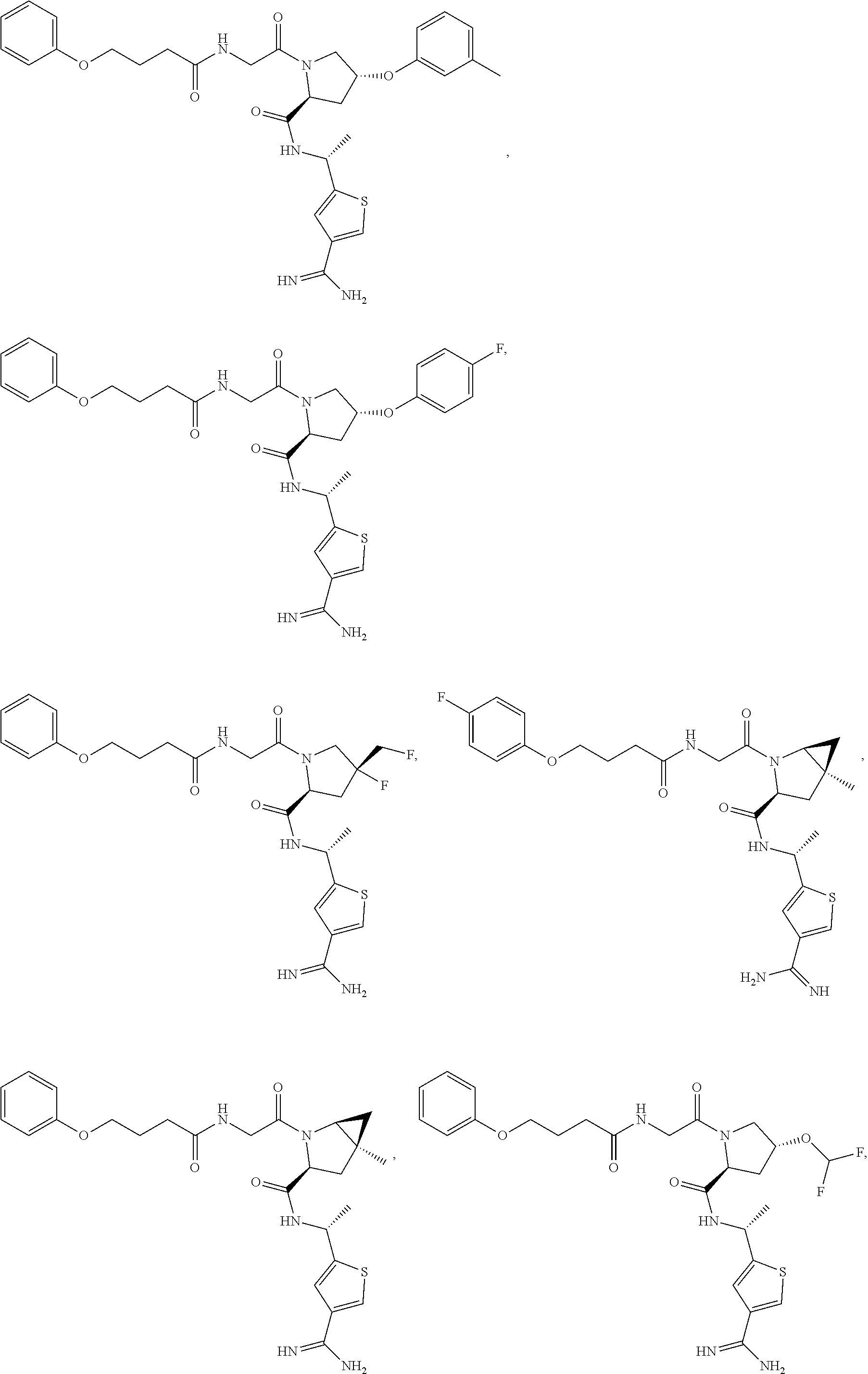 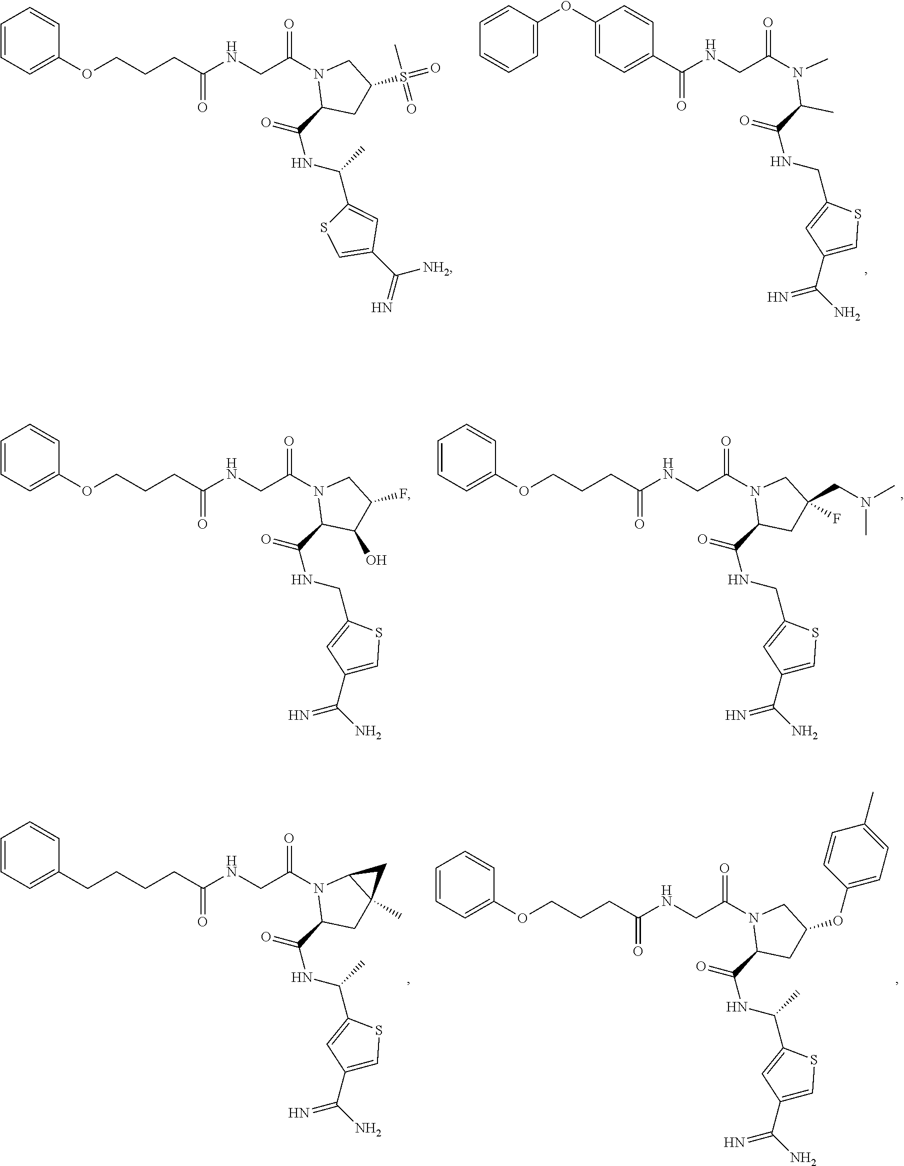 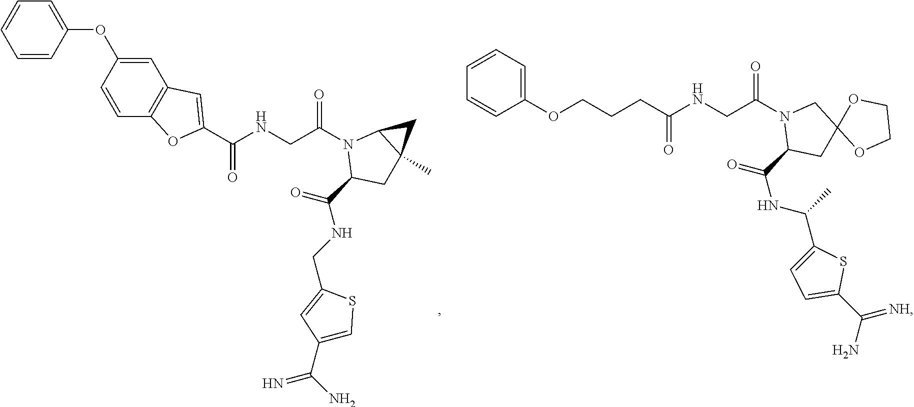 [SMILES: C[C@@]12C[C@@H]1N(C(=O)CNC(=O)c1cc3cc(Oc4ccccc4)ccc3o1)[C@H](C(=O)NCc1cc(C(=N)N)cs1)C2 ; C[C@@H](NC(=O)[C@@H]1CC2(CN1C(=O)CNC(=O)CCCOc1ccccc1)OCCO2)c1ccc(C(=N)N)s1] 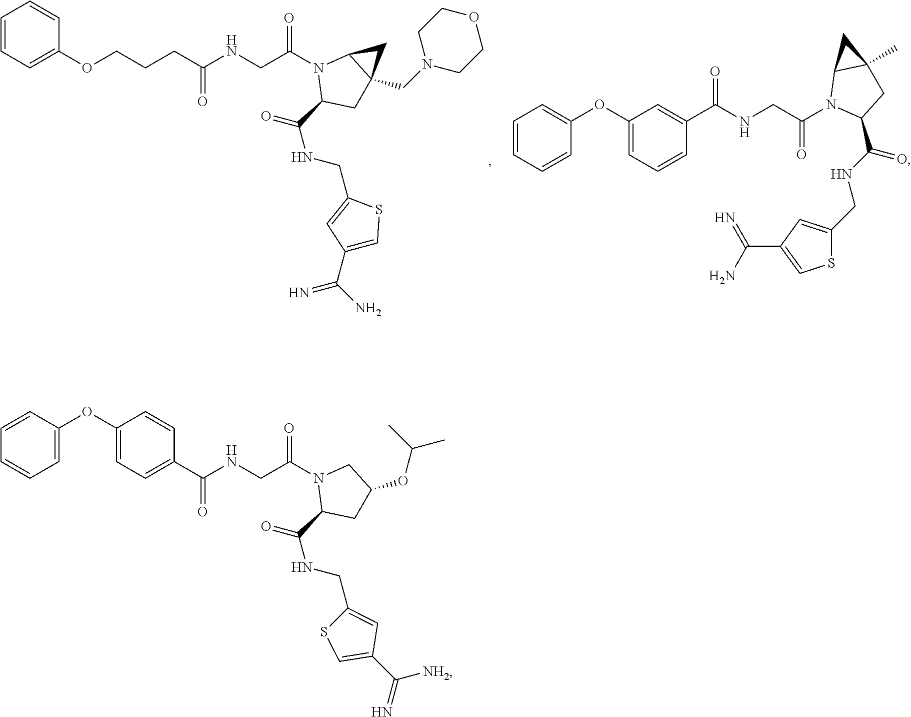 [SMILES: N=C(N)c1csc(CNC(=O)[C@@H]2C[C@]3(CN4CCOCC4)C[C@@H]3N2C(=O)CNC(=O)CCCOc2ccccc2)c1 ; C[C@@]12C[C@@H]1N(C(=O)CNC(=O)c1cccc(Oc3ccccc3)c1)[C@H](C(=O)NCc1cc(C(=N)N)cs1)C2 ; CC(C)O[C@@H]1C[C@@H](C(=O)NCc2cc(C(=N)N)cs2)N(C(=O)CNC(=O)c2ccc(Oc3ccccc3)cc2)C1] 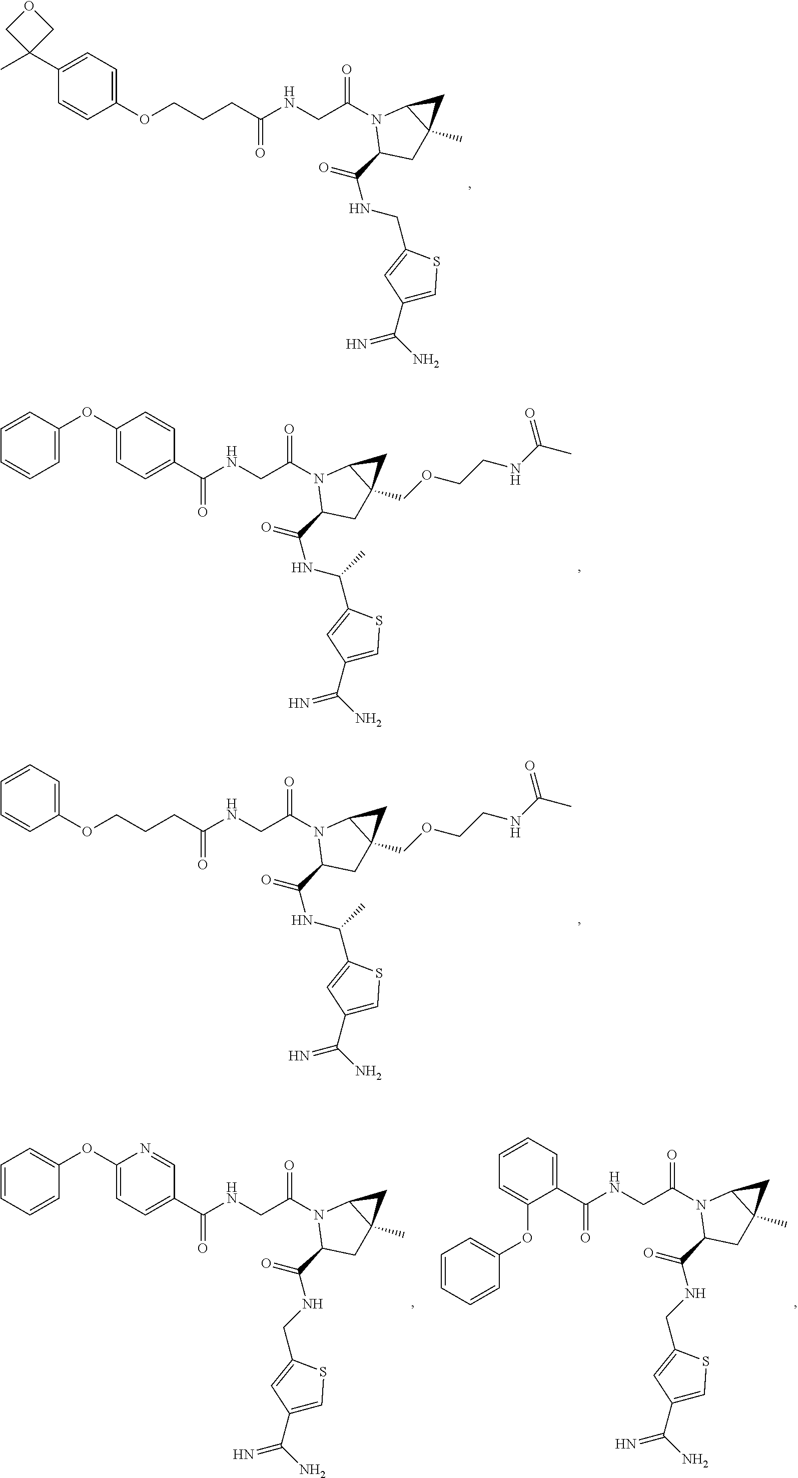 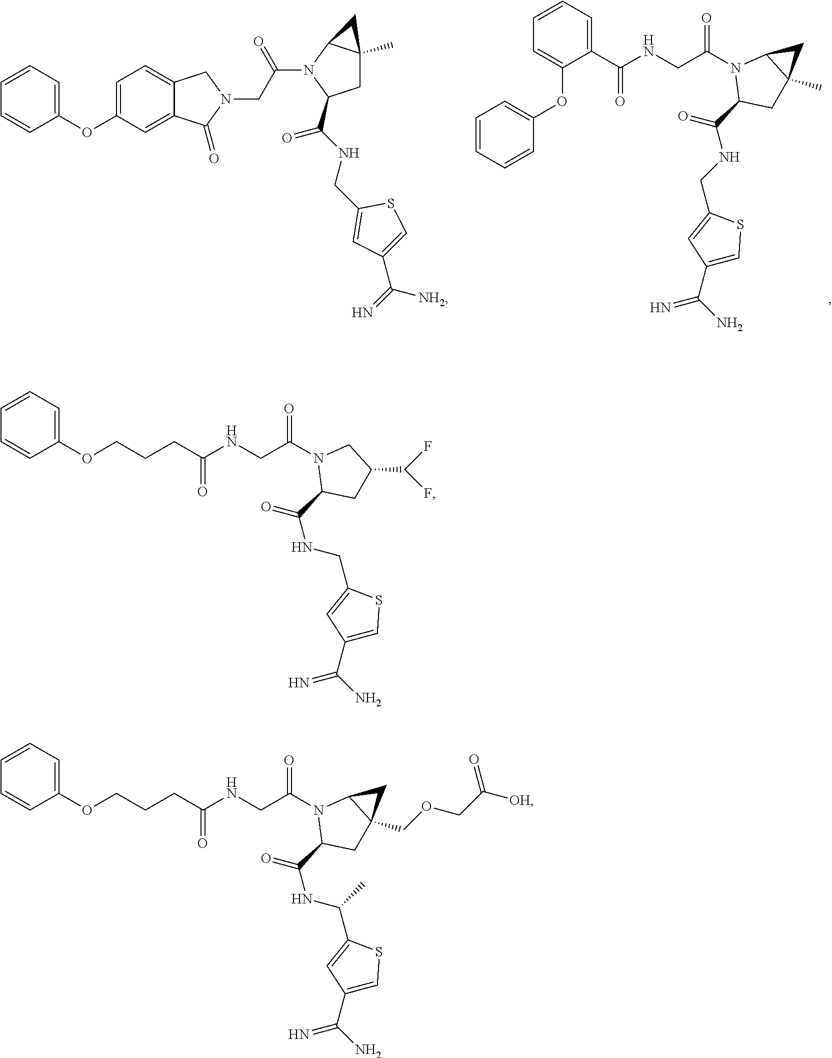 [SMILES: C[C@@]12C[C@@H]1N(C(=O)CN1Cc3ccc(Oc4ccccc4)cc3C1=O)[C@H](C(=O)NCc1cc(C(=N)N)cs1)C2 ; C[C@@]12C[C@@H]1N(C(=O)CNC(=O)c1ccccc1Oc1ccccc1)[C@H](C(=O)NCc1cc(C(=N)N)cs1)C2 ; N=C(N)c1csc(CNC(=O)[C@@H]2C[C@@H](C(F)F)CN2C(=O)CNC(=O)CCCOc2ccccc2)c1 ; C[C@@H](NC(=O)[C@@H]1C[C@]2(COCC(=O)O)C[C@@H]2N1C(=O)CNC(=O)CCCOc1ccccc1)c1cc(C(=N)N)cs1] 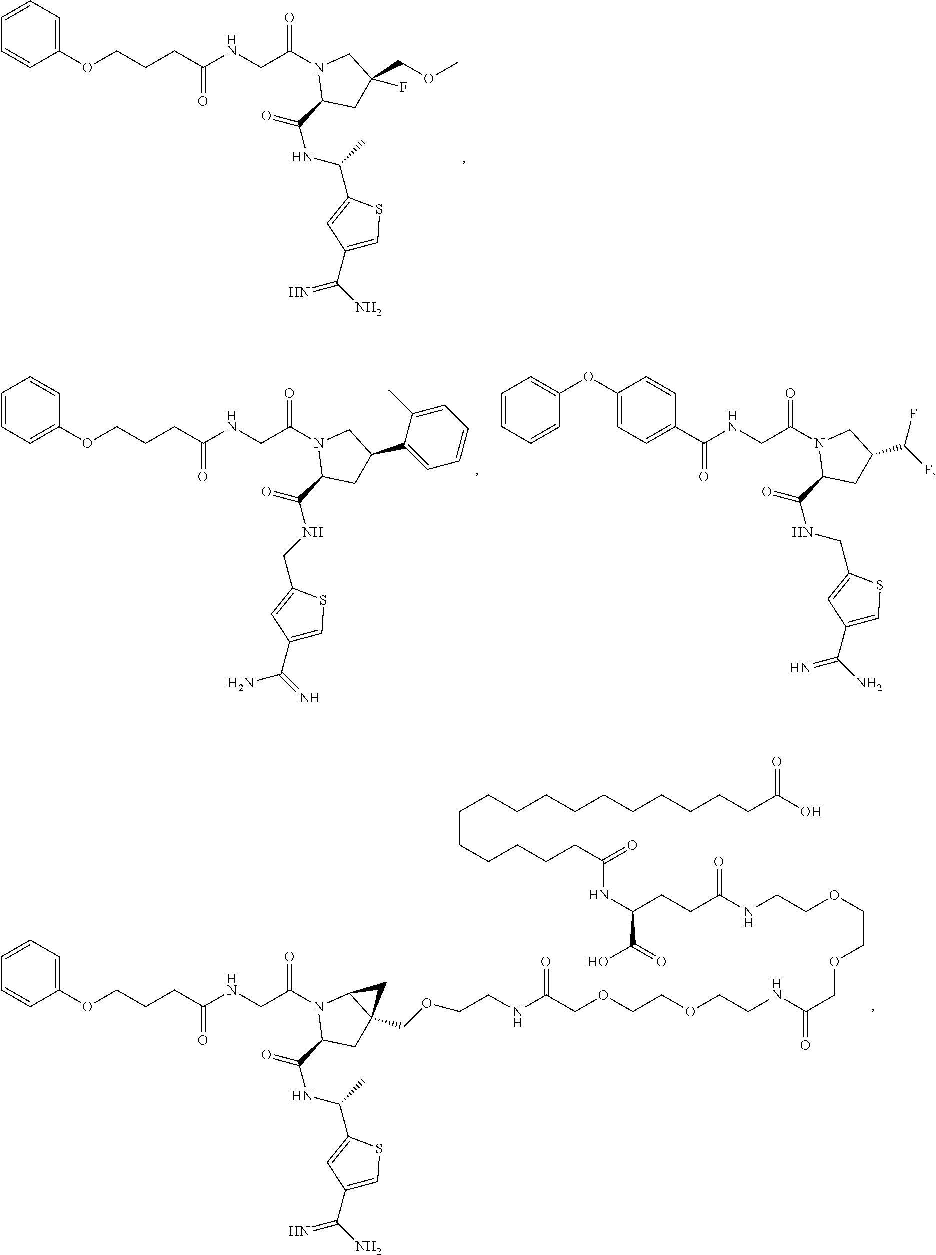 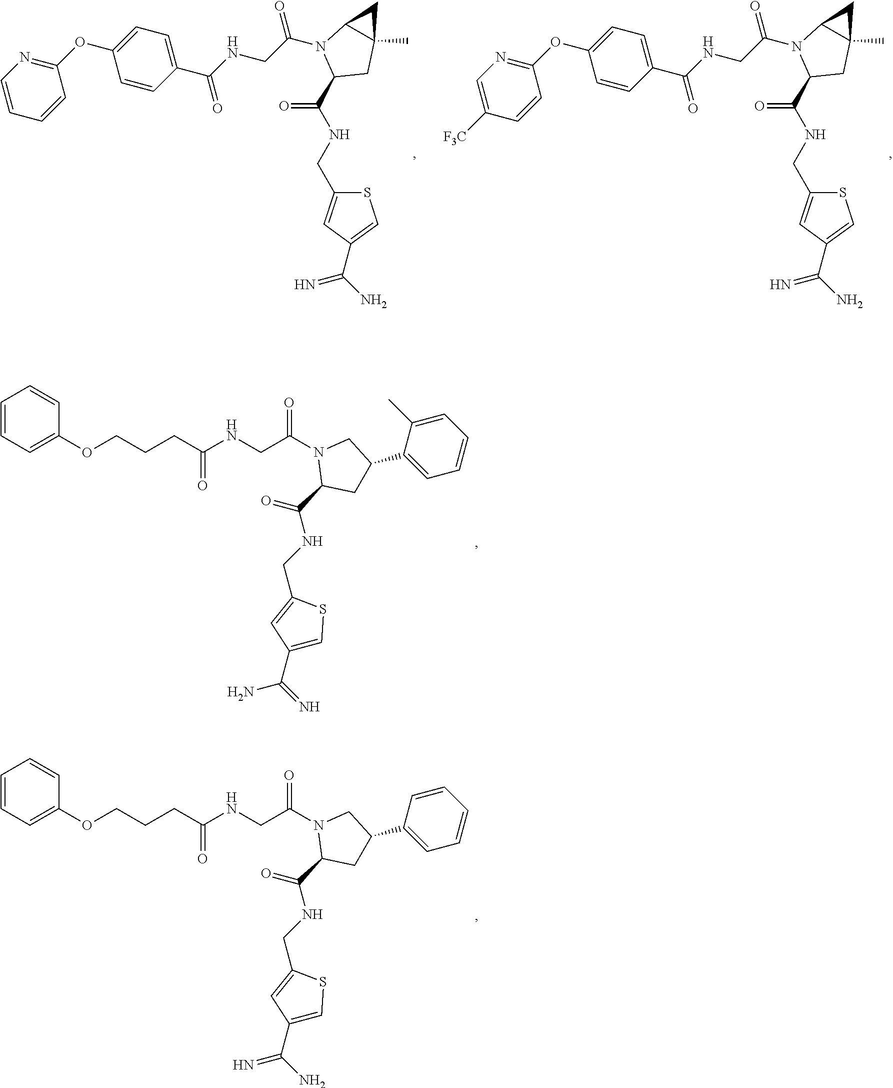 [SMILES: C[C@@]12C[C@@H]1N(C(=O)CNC(=O)c1ccc(Oc3ccccn3)cc1)[C@H](C(=O)NCc1cc(C(=N)N)cs1)C2 ; C[C@@]12C[C@@H]1N(C(=O)CNC(=O)c1ccc(Oc3ccc(C(F)(F)F)cn3)cc1)[C@H](C(=O)NCc1cc(C(=N)N)cs1)C2 ; Cc1ccccc1[C@@H]1C[C@@H](C(=O)NCc2cc(C(=N)N)cs2)N(C(=O)CNC(=O)CCCOc2ccccc2)C1 ; N=C(N)c1csc(CNC(=O)[C@@H]2C[C@@H](c3ccccc3)CN2C(=O)CNC(=O)CCCOc2ccccc2)c1] 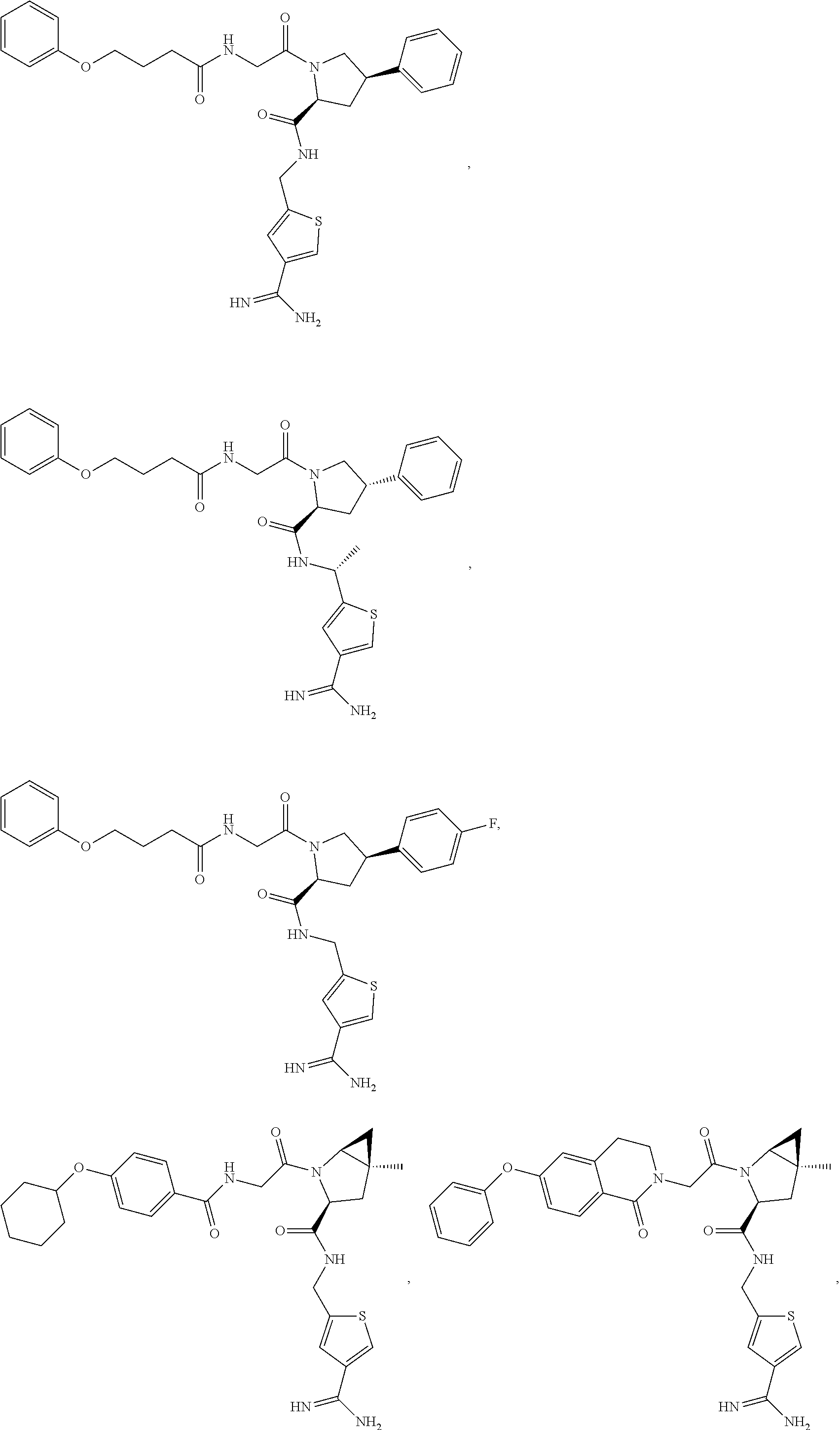 [SMILES: N=C(N)c1csc(CNC(=O)[C@@H]2C[C@H](c3ccccc3)CN2C(=O)CNC(=O)CCCOc2ccccc2)c1 ; C[C@@H](NC(=O)[C@@H]1C[C@@H](c2ccccc2)CN1C(=O)CNC(=O)CCCOc1ccccc1)c1cc(C(=N)N)cs1 ; N=C(N)c1csc(CNC(=O)[C@@H]2C[C@H](c3ccc(F)cc3)CN2C(=O)CNC(=O)CCCOc2ccccc2)c1 ; C[C@@]12C[C@@H]1N(C(=O)CNC(=O)c1ccc(OC3CCCCC3)cc1)[C@H](C(=O)NCc1cc(C(=N)N)cs1)C2 ; C[C@@]12C[C@@H]1N(C(=O)CN1CCc3cc(Oc4ccccc4)ccc3C1=O)[C@H](C(=O)NCc1cc(C(=N)N)cs1)C2] 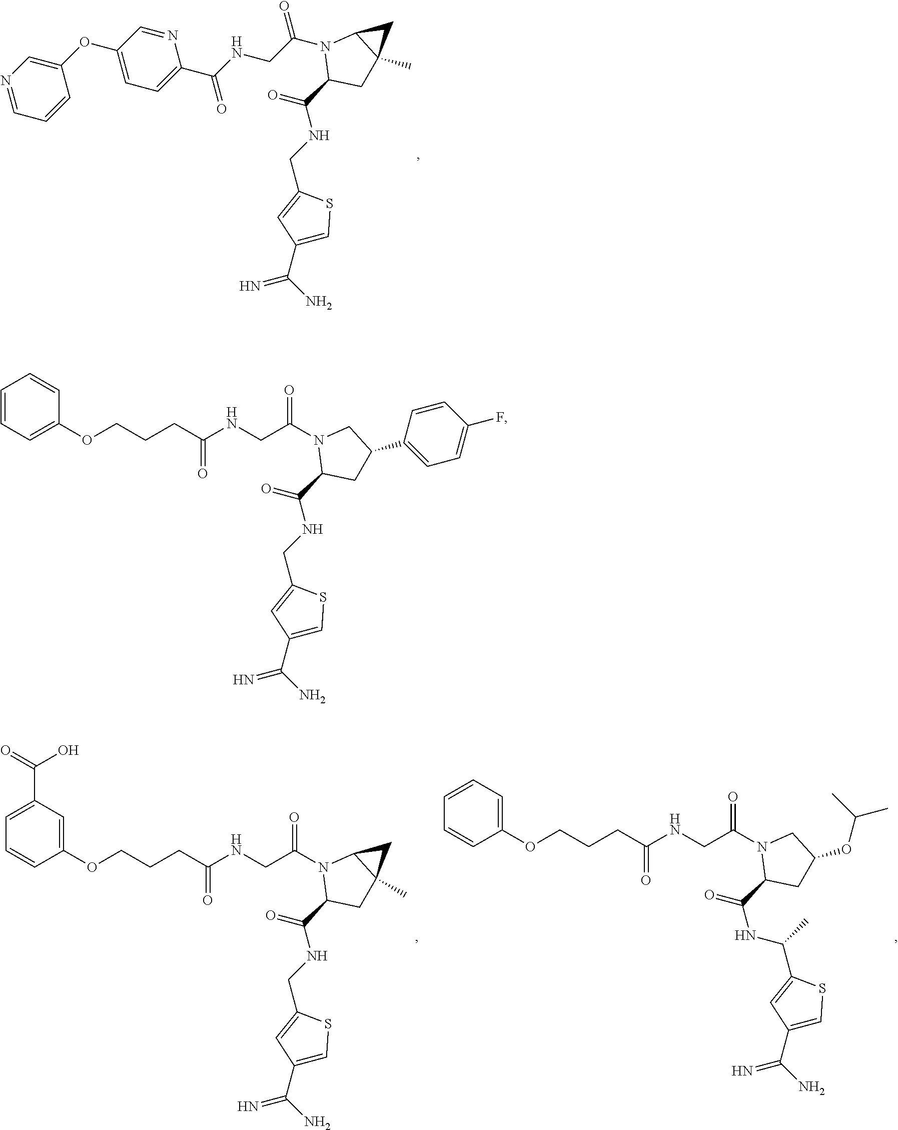 [SMILES: C[C@@]12C[C@@H]1N(C(=O)CNC(=O)c1ccc(Oc3cccnc3)cn1)[C@H](C(=O)NCc1cc(C(=N)N)cs1)C2 ; N=C(N)c1csc(CNC(=O)[C@@H]2C[C@@H](c3ccc(F)cc3)CN2C(=O)CNC(=O)CCCOc2ccccc2)c1 ; C[C@@]12C[C@@H]1N(C(=O)CNC(=O)CCCOc1cccc(C(=O)O)c1)[C@H](C(=O)NCc1cc(C(=N)N)cs1)C2 ; CC(C)O[C@@H]1C[C@@H](C(=O)N[C@H](C)c2cc(C(=N)N)cs2)N(C(=O)CNC(=O)CCCOc2ccccc2)C1] 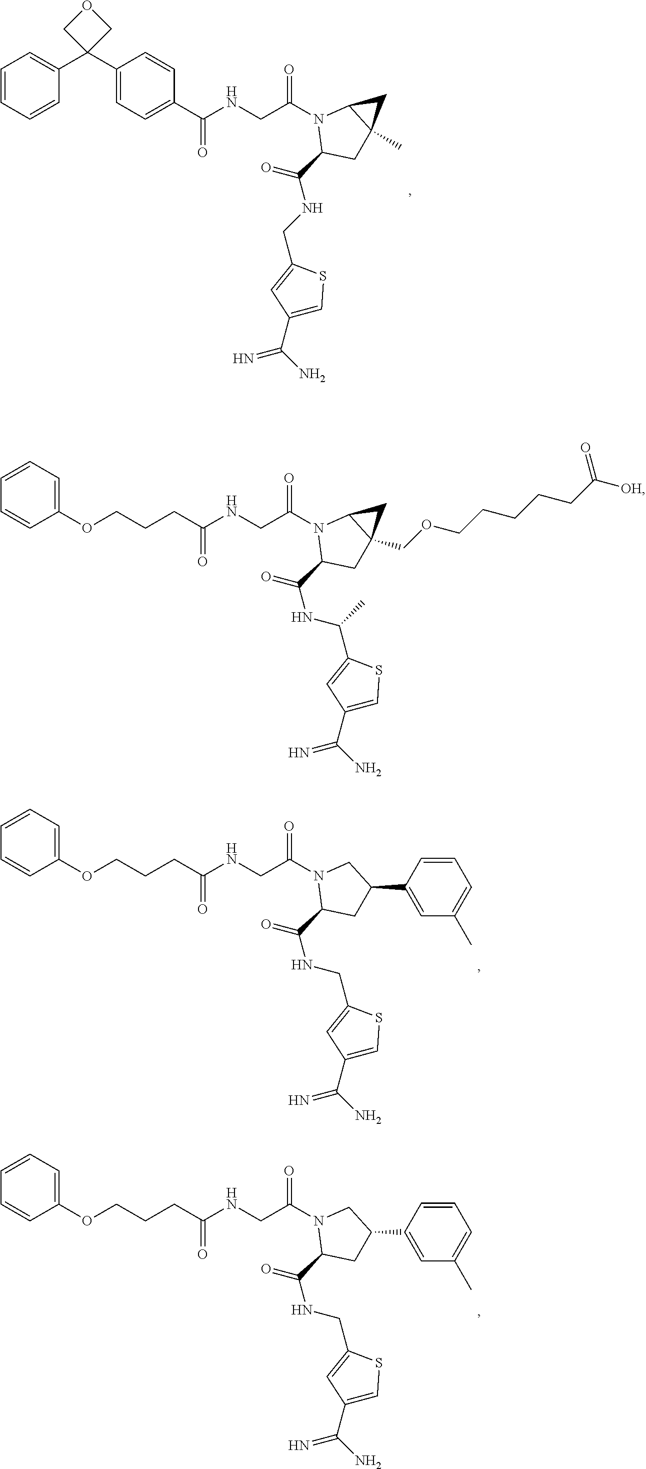 [SMILES: C[C@@]12C[C@@H]1N(C(=O)CNC(=O)c1ccc(C3(c4ccccc4)COC3)cc1)[C@H](C(=O)NCc1cc(C(=N)N)cs1)C2 ; C[C@@H](NC(=O)[C@@H]1C[C@]2(COCCCCCC(=O)O)C[C@@H]2N1C(=O)CNC(=O)CCCOc1ccccc1)c1cc(C(=N)N)cs1 ; Cc1cccc([C@H]2C[C@@H](C(=O)NCc3cc(C(=N)N)cs3)N(C(=O)CNC(=O)CCCOc3ccccc3)C2)c1 ; Cc1cccc([C@@H]2C[C@@H](C(=O)NCc3cc(C(=N)N)cs3)N(C(=O)CNC(=O)CCCOc3ccccc3)C2)c1] 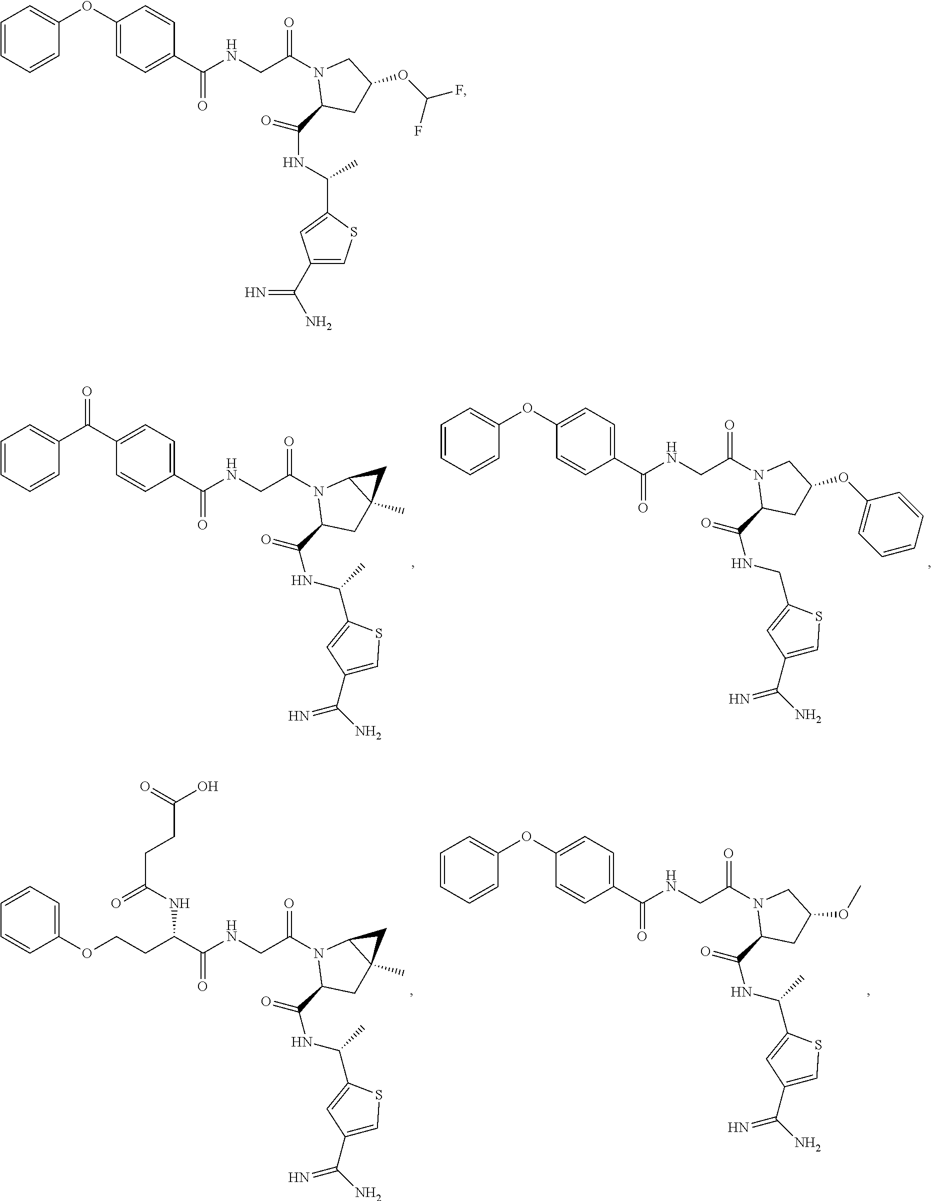 [SMILES: C[C@@H](NC(=O)[C@@H]1C[C@@H](OC(F)F)CN1C(=O)CNC(=O)c1ccc(Oc2ccccc2)cc1)c1cc(C(=N)N)cs1 ; C[C@@H](NC(=O)[C@@H]1C[C@]2(C)C[C@@H]2N1C(=O)CNC(=O)c1ccc(C(=O)c2ccccc2)cc1)c1cc(C(=N)N)cs1 ; N=C(N)c1csc(CNC(=O)[C@@H]2C[C@@H](Oc3ccccc3)CN2C(=O)CNC(=O)c2ccc(Oc3ccccc3)cc2)c1 ; C[C@@H](NC(=O)[C@@H]1C[C@]2(C)C[C@@H]2N1C(=O)CNC(=O)[C@H](CCOc1ccccc1)NC(=O)CCC(=O)O)c1cc(C(=N)N)cs1 ; CO[C@@H]1C[C@@H](C(=O)N[C@H](C)c2cc(C(=N)N)cs2)N(C(=O)CNC(=O)c2ccc(Oc3ccccc3)cc2)C1] 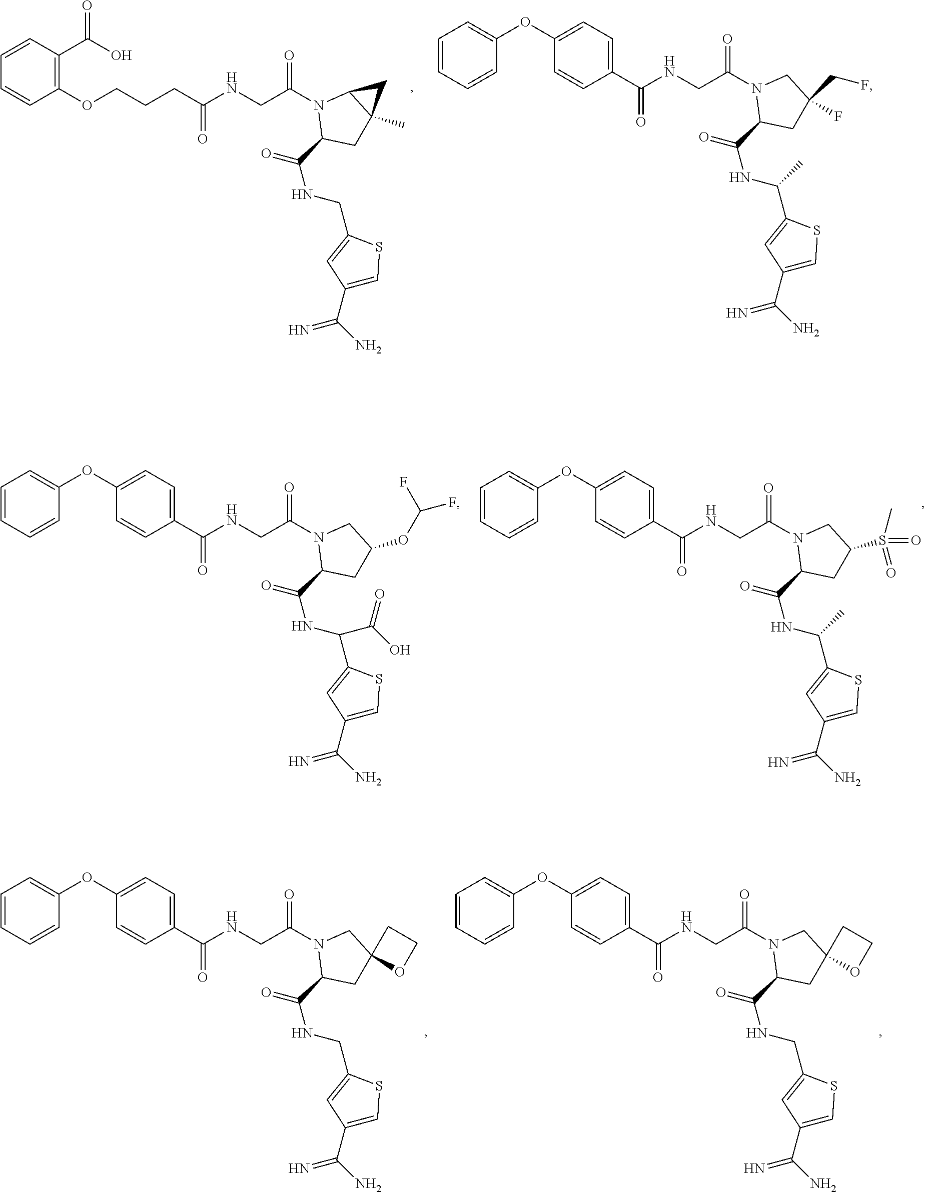 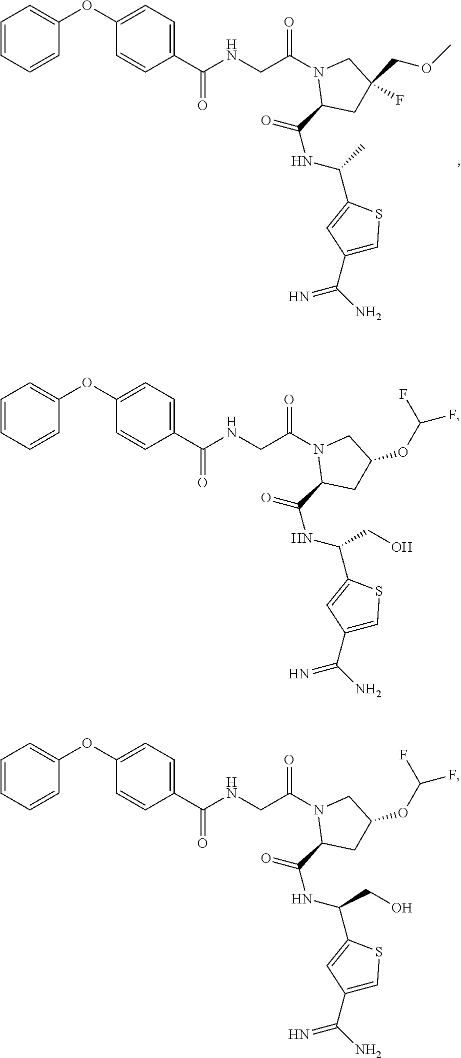 [SMILES: COC[C@@]1(F)C[C@@H](C(=O)N[C@H](C)c2cc(C(=N)N)cs2)N(C(=O)CNC(=O)c2ccc(Oc3ccccc3)cc2)C1 ; N=C(N)c1csc([C@@H](CO)NC(=O)[C@@H]2C[C@@H](OC(F)F)CN2C(=O)CNC(=O)c2ccc(Oc3ccccc3)cc2)c1 ; N=C(N)c1csc([C@H](CO)NC(=O)[C@@H]2C[C@@H](OC(F)F)CN2C(=O)CNC(=O)c2ccc(Oc3ccccc3)cc2)c1] 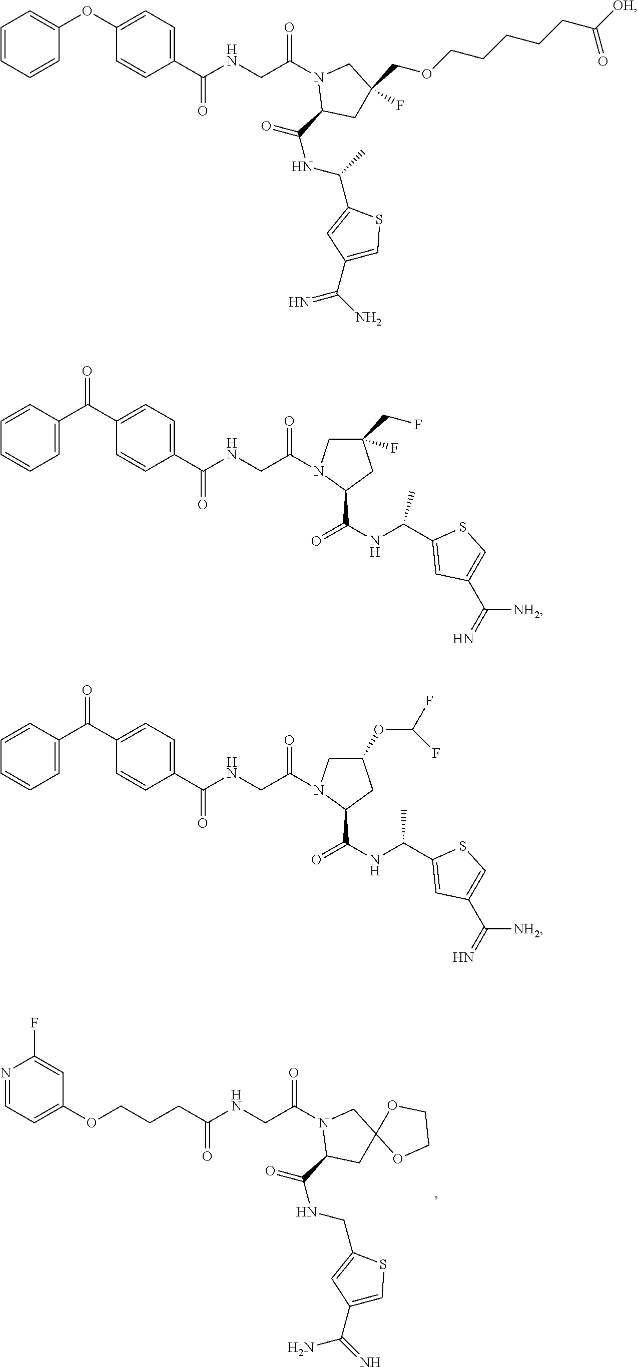 [SMILES: C[C@@H](NC(=O)[C@@H]1C[C@](F)(COCCCCCC(=O)O)CN1C(=O)CNC(=O)c1ccc(Oc2ccccc2)cc1)c1cc(C(=N)N)cs1 ; C[C@@H](NC(=O)[C@@H]1C[C@](F)(CF)CN1C(=O)CNC(=O)c1ccc(C(=O)c2ccccc2)cc1)c1cc(C(=N)N)cs1 ; C[C@@H](NC(=O)[C@@H]1C[C@@H](OC(F)F)CN1C(=O)CNC(=O)c1ccc(C(=O)c2ccccc2)cc1)c1cc(C(=N)N)cs1 ; N=C(N)c1csc(CNC(=O)[C@@H]2CC3(CN2C(=O)CNC(=O)CCCOc2ccnc(F)c2)OCCO3)c1] 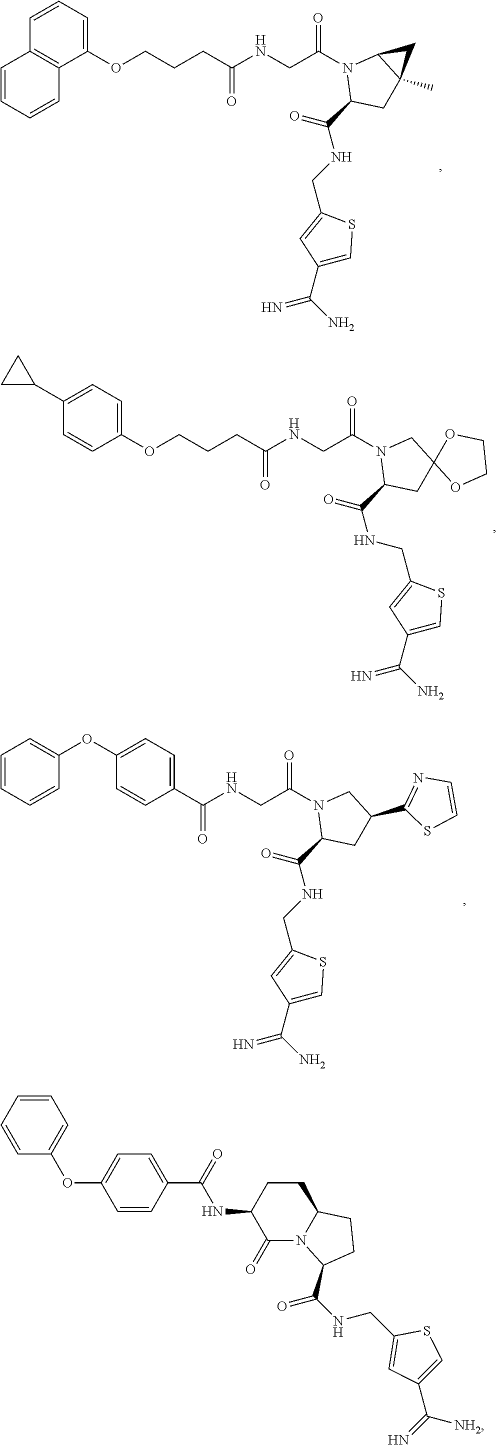 [SMILES: C[C@@]12C[C@@H]1N(C(=O)CNC(=O)CCCOc1cccc3ccccc13)[C@H](C(=O)NCc1cc(C(=N)N)cs1)C2 ; N=C(N)c1csc(CNC(=O)[C@@H]2CC3(CN2C(=O)CNC(=O)CCCOc2ccc(C4CC4)cc2)OCCO3)c1 ; N=C(N)c1csc(CNC(=O)[C@@H]2C[C@H](c3nccs3)CN2C(=O)CNC(=O)c2ccc(Oc3ccccc3)cc2)c1 ; N=C(N)c1csc(CNC(=O)[C@@H]2CC[C@@H]3CC[C@H](NC(=O)c4ccc(Oc5ccccc5)cc4)C(=O)N32)c1] 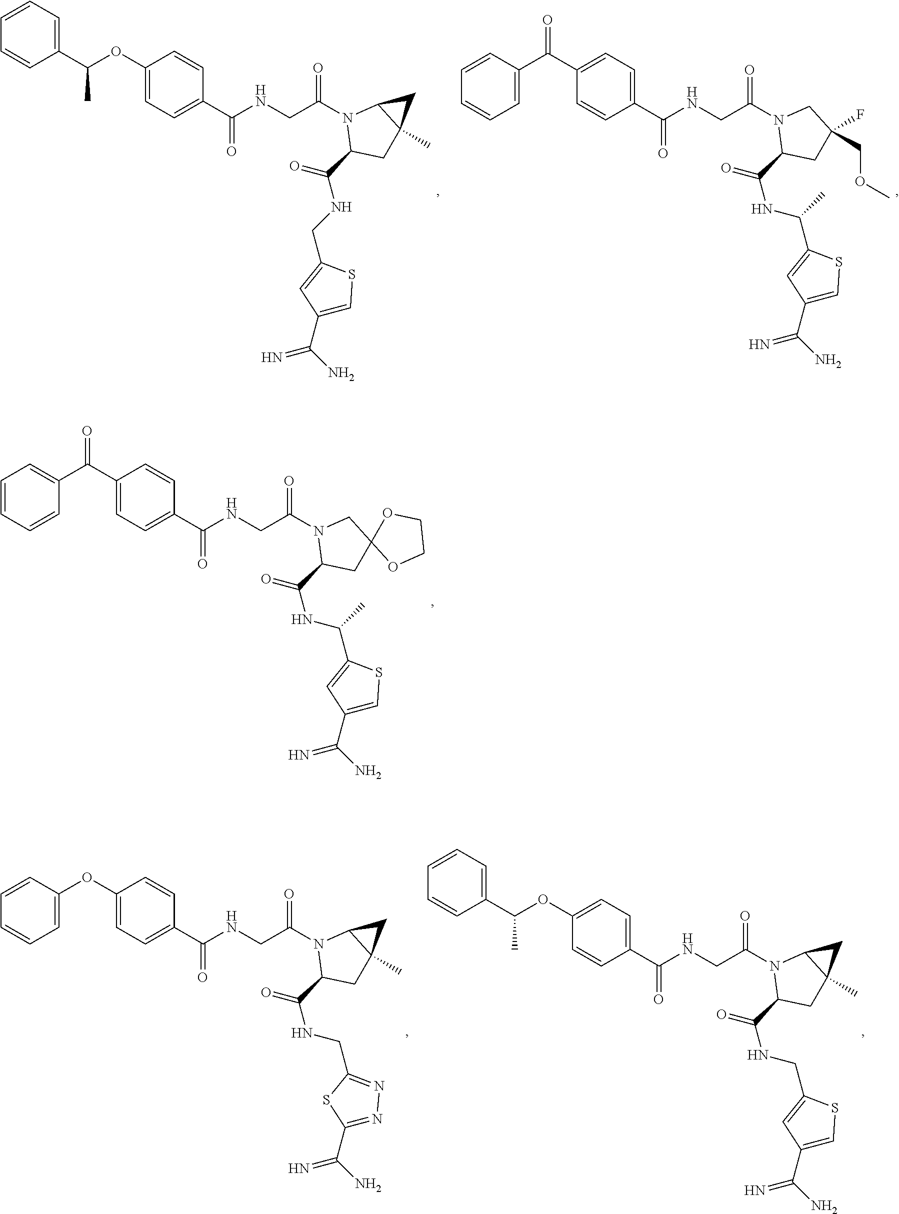 [SMILES: C[C@H](Oc1ccc(C(=O)NCC(=O)N2[C@H]3C[C@@]3(C)C[C@H]2C(=O)NCc2cc(C(=N)N)cs2)cc1)c1ccccc1 ; COC[C@@]1(F)C[C@@H](C(=O)N[C@H](C)c2cc(C(=N)N)cs2)N(C(=O)CNC(=O)c2ccc(C(=O)c3ccccc3)cc2)C1 ; C[C@@H](NC(=O)[C@@H]1CC2(CN1C(=O)CNC(=O)c1ccc(C(=O)c3ccccc3)cc1)OCCO2)c1cc(C(=N)N)cs1 ; C[C@@]12C[C@@H]1N(C(=O)CNC(=O)c1ccc(Oc3ccccc3)cc1)[C@H](C(=O)NCc1nnc(C(=N)N)s1)C2 ; C[C@@H](Oc1ccc(C(=O)NCC(=O)N2[C@H]3C[C@@]3(C)C[C@H]2C(=O)NCc2cc(C(=N)N)cs2)cc1)c1ccccc1] 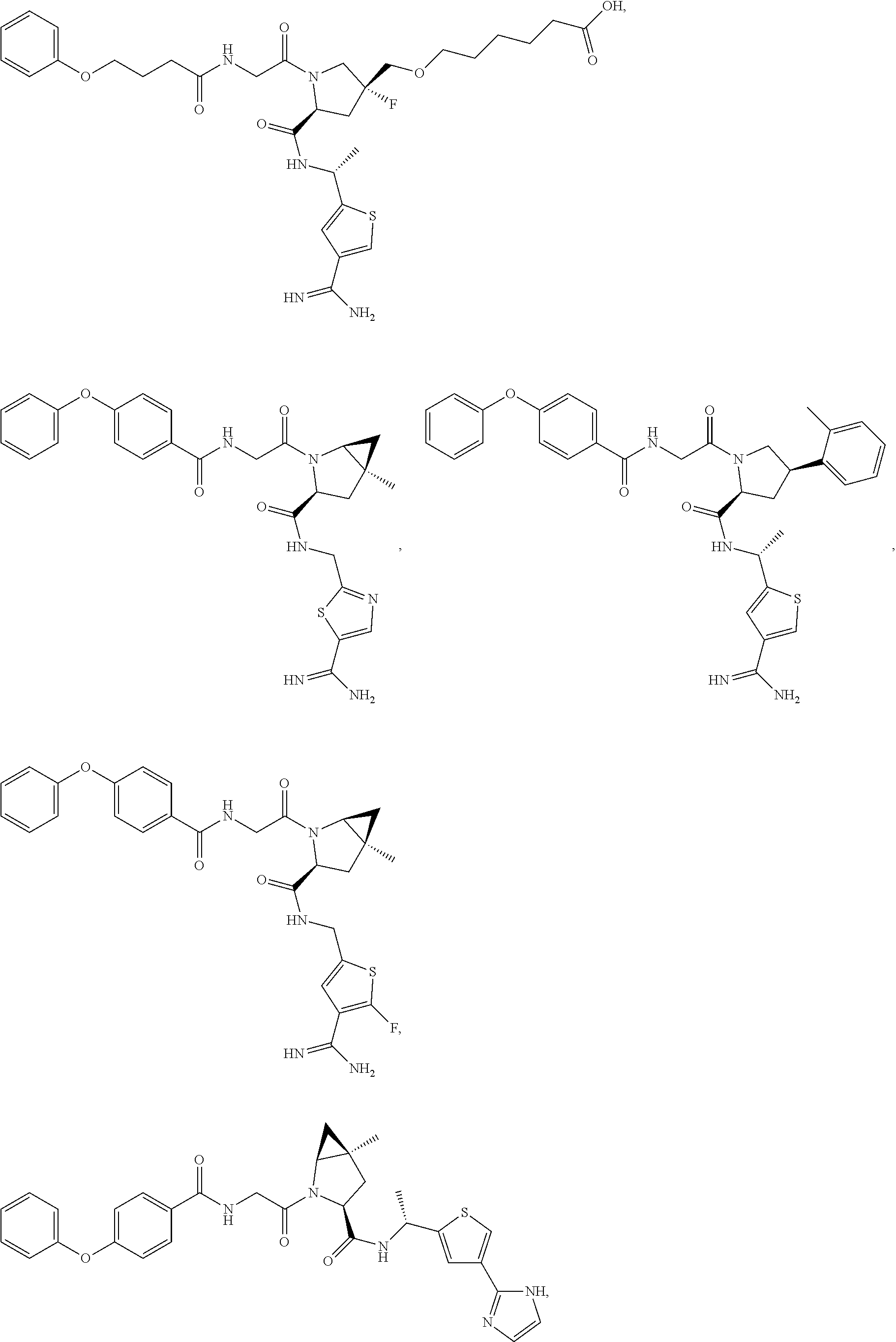 [SMILES: C[C@@H](NC(=O)[C@@H]1C[C@](F)(COCCCCCC(=O)O)CN1C(=O)CNC(=O)CCCOc1ccccc1)c1cc(C(=N)N)cs1 ; C[C@@]12C[C@@H]1N(C(=O)CNC(=O)c1ccc(Oc3ccccc3)cc1)[C@H](C(=O)NCc1ncc(C(=N)N)s1)C2 ; Cc1ccccc1[C@H]1C[C@@H](C(=O)N[C@H](C)c2cc(C(=N)N)cs2)N(C(=O)CNC(=O)c2ccc(Oc3ccccc3)cc2)C1 ; C[C@@]12C[C@@H]1N(C(=O)CNC(=O)c1ccc(Oc3ccccc3)cc1)[C@H](C(=O)NCc1cc(C(=N)N)c(F)s1)C2 ; C[C@@H](NC(=O)[C@@H]1C[C@]2(C)C[C@@H]2N1C(=O)CNC(=O)c1ccc(Oc2ccccc2)cc1)c1cc(-c2ncc[nH]2)cs1] 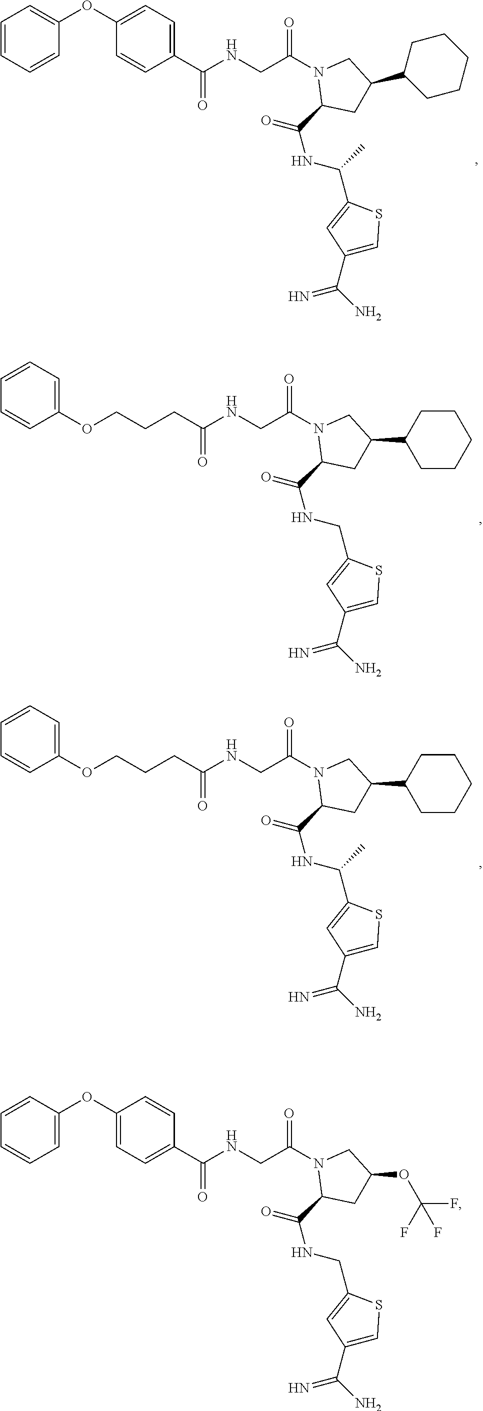 [SMILES: C[C@@H](NC(=O)[C@@H]1C[C@H](C2CCCCC2)CN1C(=O)CNC(=O)c1ccc(Oc2ccccc2)cc1)c1cc(C(=N)N)cs1 ; N=C(N)c1csc(CNC(=O)[C@@H]2C[C@H](C3CCCCC3)CN2C(=O)CNC(=O)CCCOc2ccccc2)c1 ; C[C@@H](NC(=O)[C@@H]1C[C@H](C2CCCCC2)CN1C(=O)CNC(=O)CCCOc1ccccc1)c1cc(C(=N)N)cs1 ; N=C(N)c1csc(CNC(=O)[C@@H]2C[C@H](OC(F)(F)F)CN2C(=O)CNC(=O)c2ccc(Oc3ccccc3)cc2)c1] 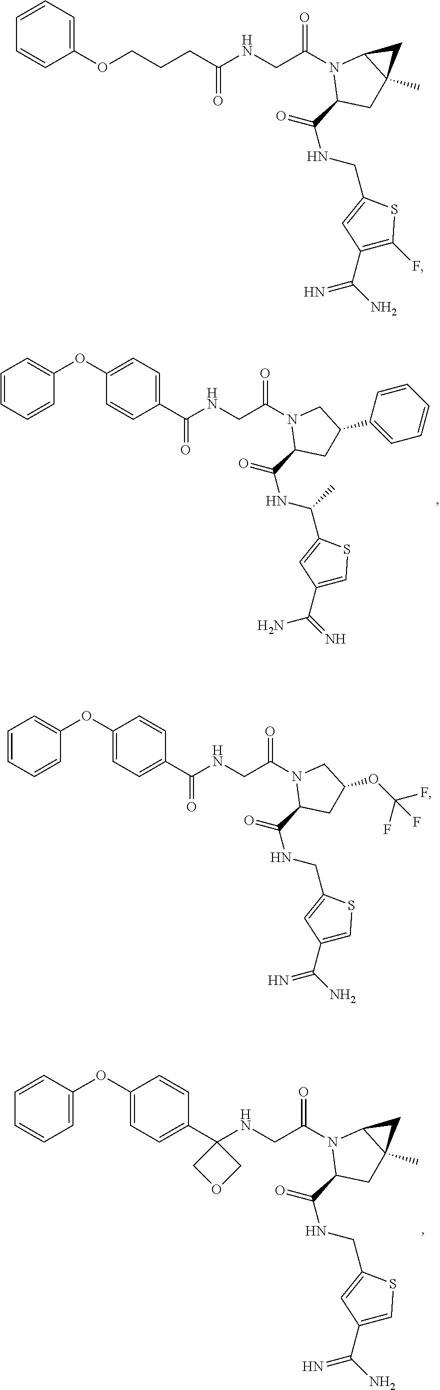 [SMILES: C[C@@]12C[C@@H]1N(C(=O)CNC(=O)CCCOc1ccccc1)[C@H](C(=O)NCc1cc(C(=N)N)c(F)s1)C2 ; C[C@@H](NC(=O)[C@@H]1C[C@@H](c2ccccc2)CN1C(=O)CNC(=O)c1ccc(Oc2ccccc2)cc1)c1cc(C(=N)N)cs1 ; N=C(N)c1csc(CNC(=O)[C@@H]2C[C@@H](OC(F)(F)F)CN2C(=O)CNC(=O)c2ccc(Oc3ccccc3)cc2)c1 ; C[C@@]12C[C@@H]1N(C(=O)CNC1(c3ccc(Oc4ccccc4)cc3)COC1)[C@H](C(=O)NCc1cc(C(=N)N)cs1)C2] 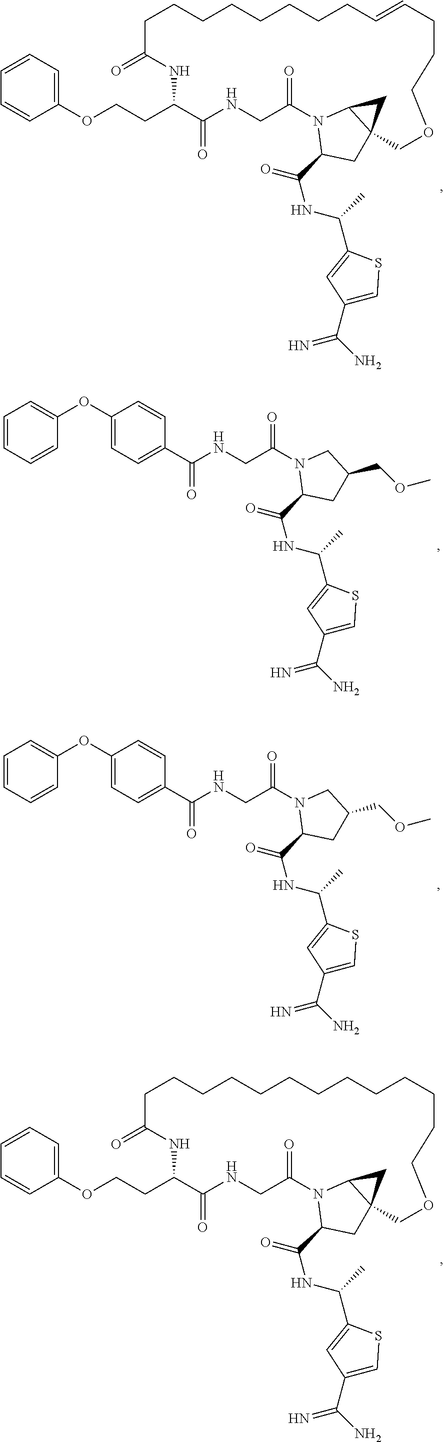 [SMILES: C[C@@H](NC(=O)[C@@H]1C[C@@]23COCCC/C=C/CCCCCCCCC(=O)N[C@@H](CCOc4ccccc4)C(=O)NCC(=O)N1[C@H]2C3)c1cc(C(=N)N)cs1 ; COC[C@H]1C[C@@H](C(=O)N[C@H](C)c2cc(C(=N)N)cs2)N(C(=O)CNC(=O)c2ccc(Oc3ccccc3)cc2)C1 ; COC[C@@H]1C[C@@H](C(=O)N[C@H](C)c2cc(C(=N)N)cs2)N(C(=O)CNC(=O)c2ccc(Oc3ccccc3)cc2)C1 ; C[C@@H](NC(=O)[C@@H]1C[C@@]23COCCCCCCCCCCCCCC(=O)N[C@@H](CCOc4ccccc4)C(=O)NCC(=O)N1[C@H]2C3)c1cc(C(=N)N)cs1] 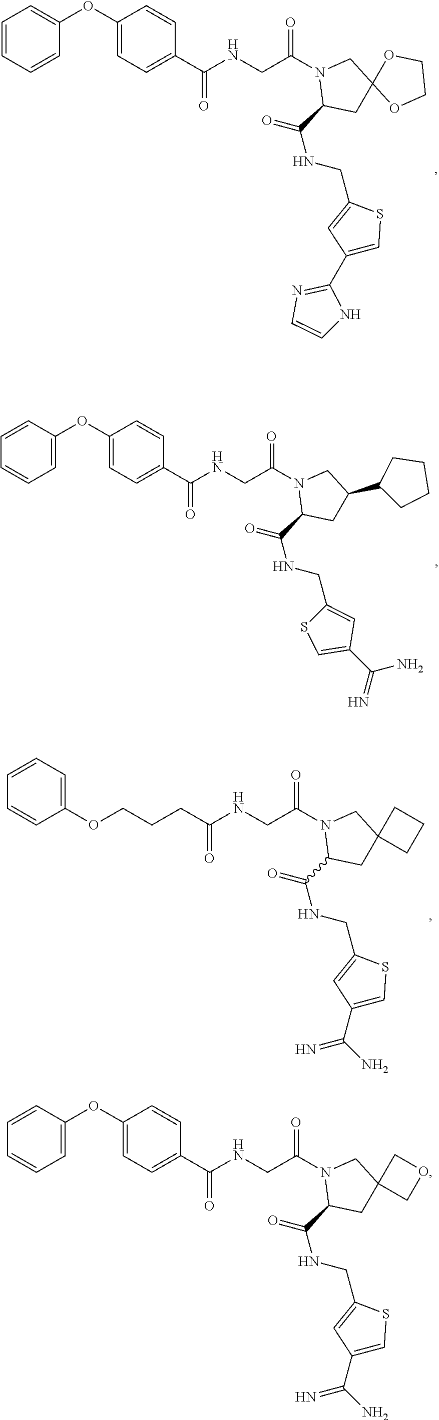 [SMILES: O=C(NCC(=O)N1CC2(C[C@H]1C(=O)NCc1cc(-c3ncc[nH]3)cs1)OCCO2)c1ccc(Oc2ccccc2)cc1 ; N=C(N)c1csc(CNC(=O)[C@@H]2C[C@H](C3CCCC3)CN2C(=O)CNC(=O)c2ccc(Oc3ccccc3)cc2)c1 ; N=C(N)c1csc(CNC(=O)C2CC3(CCC3)CN2C(=O)CNC(=O)CCCOc2ccccc2)c1 ; N=C(N)c1csc(CNC(=O)[C@@H]2CC3(COC3)CN2C(=O)CNC(=O)c2ccc(Oc3ccccc3)cc2)c1] 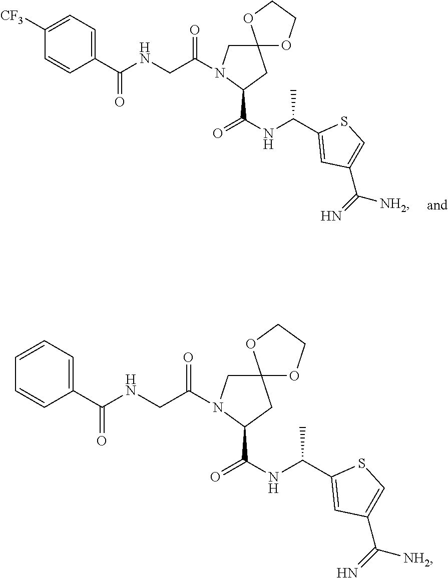 [SMILES: C[C@@H](NC(=O)[C@@H]1CC2(CN1C(=O)CNC(=O)c1ccc(C(F)(F)F)cc1)OCCO2)c1cc(C(=N)N)cs1 ; C[C@@H](NC(=O)[C@@H]1CC2(CN1C(=O)CNC(=O)c1ccccc1)OCCO2)c1cc(C(=N)N)cs1]
or a pharmaceutically acceptable salt thereof.

59. A pharmaceutical composition comprising a compound of claim 1, or a pharmaceutically acceptable salt thereof, and a pharmaceutically acceptable carrier.

60. A method of treating a disorder mediated by C1s, comprising administering to a human subject in need thereof a therapeutically effective amount of a compound of claim 1 or a pharmaceutically acceptable salt thereof.

61-62. (canceled)

63. The method of claim 1, wherein the disorder is C3 glomerulopathy, an ophthalmic disorder, age-related macular degeneration (AMD), paroxysmal nocturnal hemoglobinuria, angioderma, hereditary angioedema, autoimmune hemolytic anemia, or cold agglutinin disease, hereditary angioedema type 1, hereditary angioedema type 2, trauma, inflammation, sepsis, multiple organ dysfunction syndrome, endotoxemia, end stage renal disease, kidney failure, delayed graft function, ischemic reperfusion injury, neuromyelitis optica, common variable immunodeficiency, antibody-mediated rejection, graft rejection, asthma, allergic asthma, angioneurotic edema, acute ACE-induced angioedema, kidney transplantation, and acute kidney injury.

64. (canceled)

65. A pharmaceutical composition comprising a compound of claim 58, or a pharmaceutically acceptable salt thereof, and a pharmaceutically acceptable carrier.

66. A method of treating a disorder mediated by C1s, comprising administering to a human subject in need thereof a therapeutically effective amount of a compound of claim 58 or a pharmaceutically acceptable salt thereof.
